(19) United States
(12) Patent Application Publication
(10) Pub. No.: US 2025/0353851 A1
(43) Pub. Date: Nov. 20, 2025

(54) PURINES AND METHODS OF THEIR USE

(71) Applicant: Kineta, Inc., Seattle, WA (US)

(72) Inventors: Gnanasambandam Kumaravel, Lexington, MA (US); Madeline Macdonnell, Brighton, MA (US); Hairuo Peng, Needham, MA (US); Kerem Ozboya, Cambridge, MA (US); Iwona Wrona, Sharon, MA (US); Bertrand Le Bourdonnec, Northborough, MA (US); Matthew Lucas, Lexington, MA (US); Vanessa Kuria, Somerville, MA (US); Byron DeLaBarre, Arlington, MA (US)

(21) Appl. No.: 18/717,174

(22) PCT Filed: Dec. 7, 2022
(86) PCT No.: PCT/US2022/052118

§ 371 Date: Jun. 6, 2024

Related U.S. Application Data
(60) Provisional application No. 63/287,522, filed on Dec. 8, 2021.

(51) Int. Cl. C07D 473/34; A61K 31/52; A61K 31/5377; A61K 31/5383; A61K 31/5386; A61K 31/675; C07D 401/14; C07D 473/00; C07D 473/16; C07D 487/04; C07D 487/14; C07D 491/147; C07D 495/04; C07D 498/04; C07D 498/14; C07D 513/04; C07D 519/00; C07F 9/6561

(52) U.S. Cl. CPC C07D 473/34; A61K 31/52; A61K 31/5377; A61K 31/5383; A61K 31/5386; A61K 31/675; C07D 401/14; C07D 473/00; C07D 473/16; C07D 487/04; C07D 487/14; C07D 491/147; C07D 495/04; C07D 498/04; C07D 498/14; C07D 513/04; C07D 519/00; C07F 9/65616

(57) ABSTRACT

Disclosed are bicyclic heteroarene compounds, including purines, as PIKfyve inhibitors useful in the treatment of a TDP-43-associated neurological/neurodegenerative disorder, such as frontotemporal dementia, ALS and Alzheimer's disease. The compounds described herein, alone or in combination with other pharmaceutically active agents, can be used for treating or preventing such neurological/neurodegenerative diseases.

### FIELD OF THE INVENTION

The invention relates to bicyclic heteroarenes and their use for therapeutic treatment of neurological disorders in patients, such as human patients.

### BACKGROUND

An incomplete understanding of the molecular perturbations that cause disease, as well as a limited arsenal of robust model systems, has contributed to a failure to generate successful disease-modifying therapies against common and progressive neurological disorders, such as ALS and FTD. Progress is being made on many fronts to find agents that can arrest the progress of these disorders. However, the present therapies for most, if not all, of these diseases provide very little relief. Accordingly, a need exists to develop therapies that can alter the course of neurodegenerative diseases. More generally, a need exists for better methods and compositions for the treatment of neurodegenerative diseases in order to improve the quality of the lives of those afflicted by such diseases.

### SUMMARY

TDP-43 is a nuclear DNA/RNA binding protein involved in RNA splicing. Under pathological cell stress, TDP-43 translocates to the cytoplasm and aggregates into stress granules and related protein inclusions. These phenotypes are hallmarks of degenerating motor neurons and are found in 97% of all ALS cases. The highly penetrant nature of this pathology indicates that TDP-43 is broadly involved in both familial and sporadic ALS. Additionally, TDP-43 mutations that promote aggregation are linked to higher risk of developing ALS, suggesting protein misfolding and aggregation act as drivers of toxicity. TDP-43 toxicity can be recapitulated in yeast models, where the protein induces a viability deficit and localizes to stress granules.

In an aspect, the invention provides a compound of formula (1)

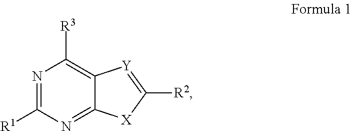

or a pharmaceutically acceptable salt thereof,
where
X is NR^{A}.
Y is CR^{A} or N;
R¹ is optionally substituted C₁-C₁₀ heteroaryl including a 5-membered ring having a nitrogen atom at position 2 relative to the bond to the core; 4,5-dihydropyrazol-1-yl substituted with phenyl; optionally substituted pyrimidin-2-yl, optionally substituted pyridazin-6-yl, optionally substituted pyrimidin-4-yl; pyridin-3-yl optionally substituted with methoxy; optionally substituted indazol-1-yl; optionally substituted indazol-2-yl; optionally substituted indazol-7-yl; optionally substituted isoindolin-6-yl; optionally substituted pyridazin-5-yl; optionally substituted pyrrolidine-1-yl; optionally substituted pyrimidin-6-yl; optionally substituted piperazinyl; phenyl substituted with methoxy, optionally substituted C₁-C₆ alkyl, hydroxyl, optionally substituted C₂-C₉ heteroaryl, optionally substituted C₆-C₁₀ aryl, optionally substituted C₂-C₉ heterocyclyl, or C₃-C₈ cycloalkoxy; optionally substituted C₃ carbocyclyl; optionally substituted morpholin-1-yl; optionally substituted benzodioxolyl; optionally substituted benzopyrrolidonyl; optionally substituted tetrahydroquinoline; optionally substituted monoalkylamino; optionally substituted dialkylamino; amino monosubstituted with optionally substituted C₂-C₉ heteroaryl; halo; optionally substituted C₂-C₉ heterocycle C₁ alkyl; optionally substituted C₂-C₉ heteroaryl C₁ alkyl; optionally substituted benzodioxanyl; —NHNHR^{1A}; —N(R^{1A})N═C(R^{1B})₂; —C(R^{1A})═N—N(R^{1B})₂; —C(R^{1A})═NOR^{1A}; or Q¹-N(R^{1c})₂;
Q¹ is a bond, CH₂, or CO;
each R^{1A} is independently H, optionally substituted C₁-C₆ alkyl, optionally substituted C₆-C₁₀ aryl, or optionally substituted C₆-C₁₀ aryl C₁-C₆ alkyl;
one R^{1B} is independently H, optionally substituted C₁-C₆ alkyl, optionally substituted C₆-C₁₀ aryl, or optionally substituted C₂-C₉ heteroaryl; and the remaining R^{1B} is optionally substituted C₁-C₆ alkyl, optionally substituted C₆-C₁₀ aryl, or optionally substituted C₂-C₉ heteroaryl;
each R^{1C} is independently H, optionally substituted C₁-C₆ alkyl, optionally substituted C₆-C₁₀ aryl, optionally substituted C₃-C₈ cycloalkyl, or optionally substituted C₂-C₉ heteroaryl; or both Ric, together with the nitrogen atom to which they are attached, combine to form C₂-C₉ heterocyclyl or C₂-C₉ heteroaryl;
R² is H, halogen, optionally substituted C₆-C₁₀ aryl; optionally substituted C₁₋₉ heterocyclyl; —O-pyridin-3-yl; optionally substituted C₃-C₈ cycloalkyl; optionally substituted C₃-C₈ cycloalkenyl, C₁-C₂ alkyl optionally substituted with hydroxy, methoxy, —CH₂OH, pyridin-4-yl, 4-pyridon-1-yl, —O-pyridin-4-yl, oxo, or dialkyl amino; C₁ alkyl optionally substituted with deuterium, oxo, hydroxy, halo, or amino substituted with C₃ cycloalkyl; C₃ alkyl substituted with hydroxy, oxo, or dialkyl amino; C₄ alkyl; optionally substituted C₂-C₉ heteroaryl; -Q-N(R^{1c})₂; —S(O)₁—R^{1A}; or —P(O)(R^{1A})₂; and each R^{A} is independently H, C₁-C₂ alkyl optionally substituted with hydroxyl or —S(O)₁— (optionally substituted C₁-C₆ alkyl), C₃ alkyl, C₄-C₅ alkyl substituted with hydroxyl, optionally substituted C₂-C₉ heteroaryl C₁-C₆ alkyl; optionally substituted C₃-C₈ cycloalkyl, optionally substituted C₃-C₈ cycloalkyl C₁-C₆ alkyl, optionally substituted C₆-C₁₀ aryl, or optionally substituted C₂-C₉ heteroaryl; or R² and R^{A}, together with the atoms to which they are attached, combine to form an optionally substituted C₃-C₄ heterocyclic ring, and the remaining R^{A}, if present, is H, C₁-C₂ alkyl optionally substituted with hydroxyl or —S(O)₁— (optionally substituted C₁-C₆ alkyl), C₃ alkyl, C₄-C₅ alkyl substituted with hydroxyl, optionally substituted C₂-C₉ heteroaryl C₁-C₆ alkyl; optionally substituted C₃-C₈ cycloalkyl, optionally substituted C₃-C₈ cycloalkyl C₁-C₆ alkyl, optionally substituted C₆-C₁₀ aryl, or optionally substituted C₂-C₉ heteroaryl;
r is 0, 1, or 2; and
R³ is

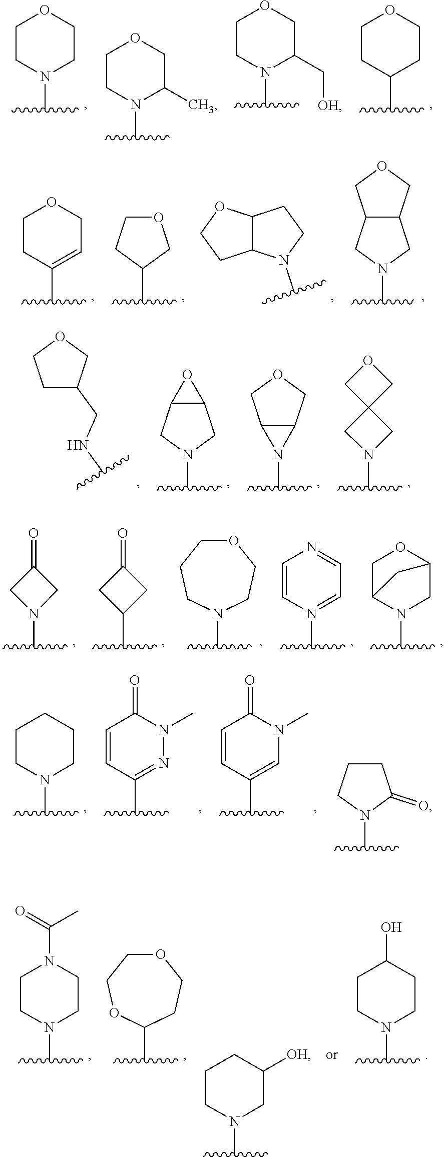

In some embodiments, X is NR^{A}. In some embodiments, Y is N. In some embodiments, R³ is

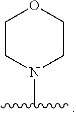 [SMILES: *N1CCOCC1]

In some embodiments, R³ is

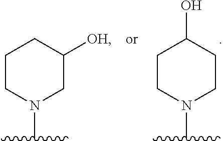 [SMILES: *N1CCCC(O)C1 ; *N1CCC(O)CC1]

In some embodiments, the compound is of formula 1a:

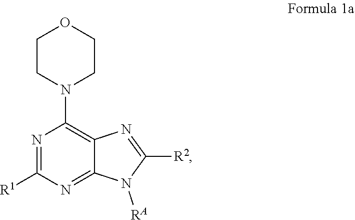

or a pharmaceutically acceptable salt thereof.

In some embodiments, R^{A} is C₁-C₂ alkyl optionally substituted with hydroxyl or —S(O)CH₃, C₃ alkyl, C₄-C₅ alkyl substituted with hydroxyl. In some embodiments, R^{A} is H. In some embodiments, R¹ is optionally substituted C₂-C₉ heteroaryl including a 5-membered ring having a nitrogen atom at position 2 relative to the bond to the core. In some embodiments, R¹ is optionally substituted pyrazol-1-yl, optionally substituted pyrazol-3-yl, optionally substituted 1,2,3-triazol-1-yl, optionally substituted 1,2,3-traizol-2-yl, optionally substituted benzotriazole-1-yl, optionally substituted 1,2,4 triazol-3-yl, optionally substituted 1,2,4-oxadizol-3-yl, or optionally substituted 1,2,4-oxadizol-2-yl. In some embodiments, R¹ is pyrazol-1-yl substituted at position 3. In some embodiments, R¹ is pyrazol-1-yl substituted at position 4. In some embodiments, R¹ is optionally substituted with optionally substituted C₆-C₁₀ aryl, optionally substituted C₁-C₆ alkyl, optionally substituted C₁-C₆ heteroalkyl, optionally substituted C₁₋₉ heterocyclyl, optionally substituted C₂-C₉ heteroaryl, or optionally substituted C₃₋₈ cycloalkyl, or halo (e.g., chloro, fluoro, bromo, iodo). In some embodiments, R¹ is

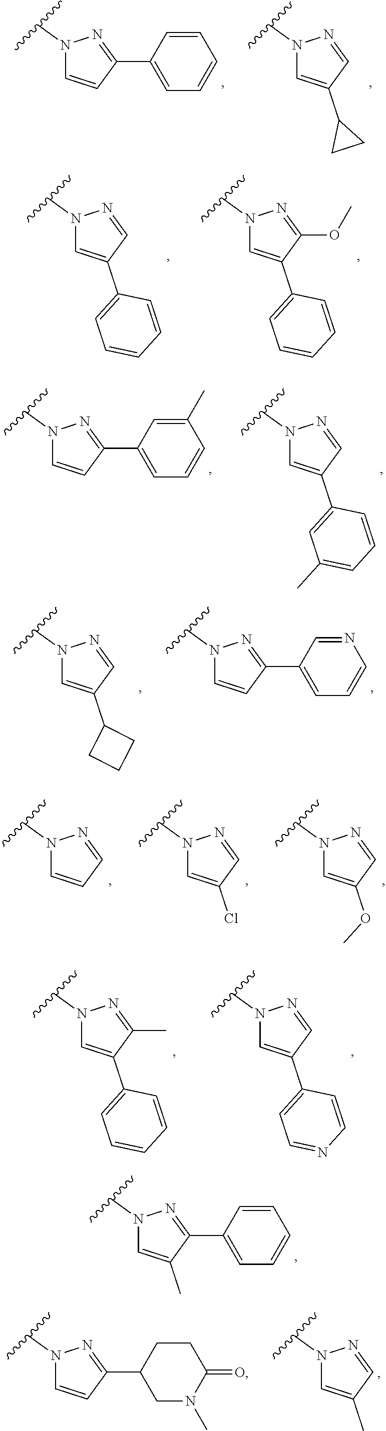

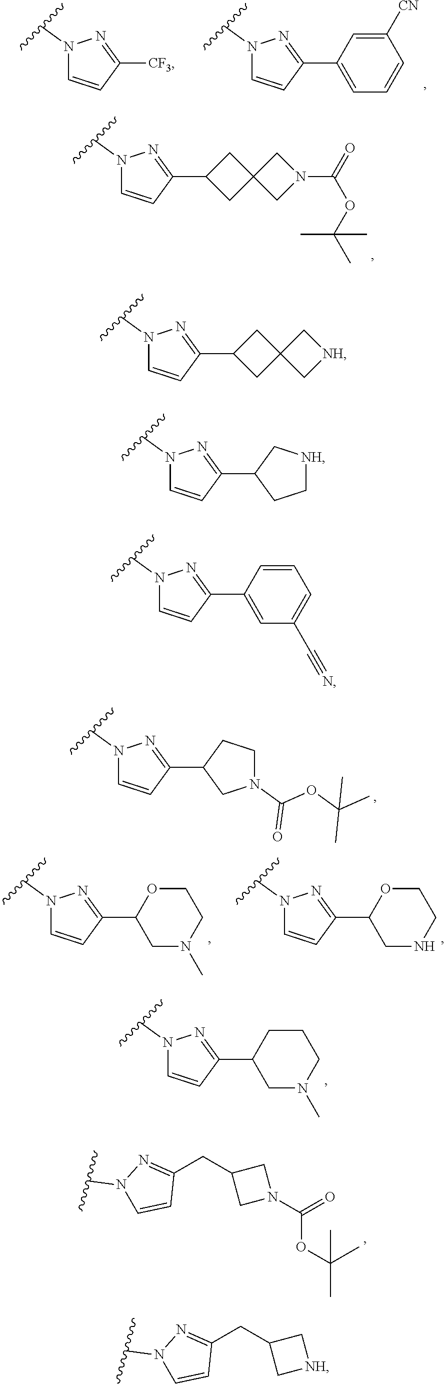

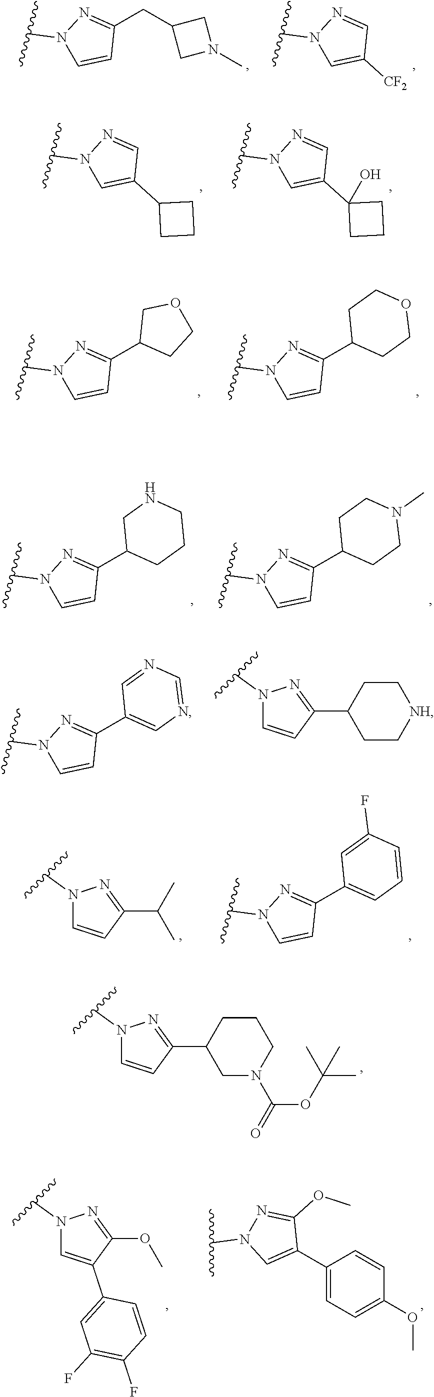

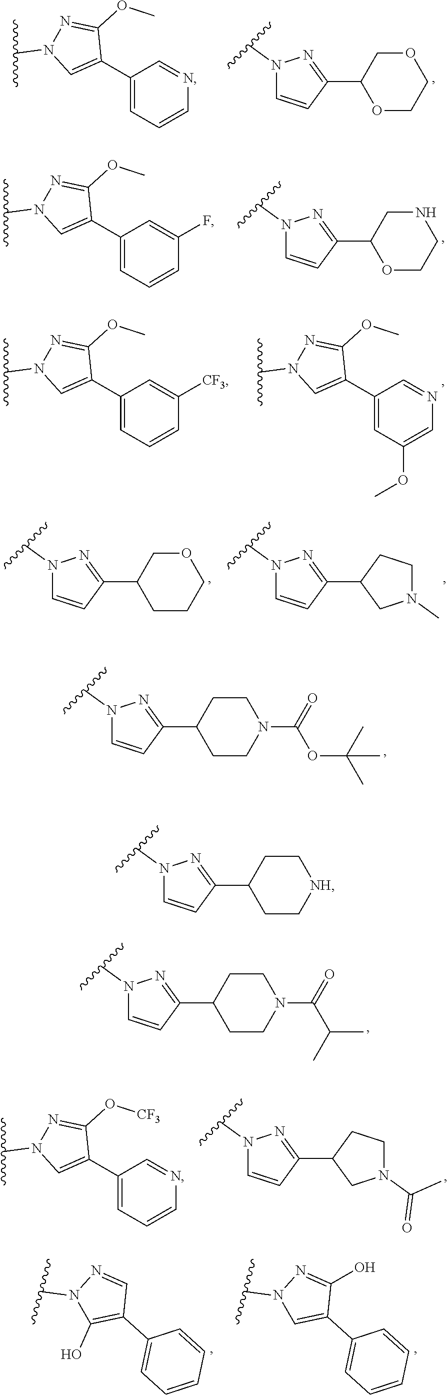

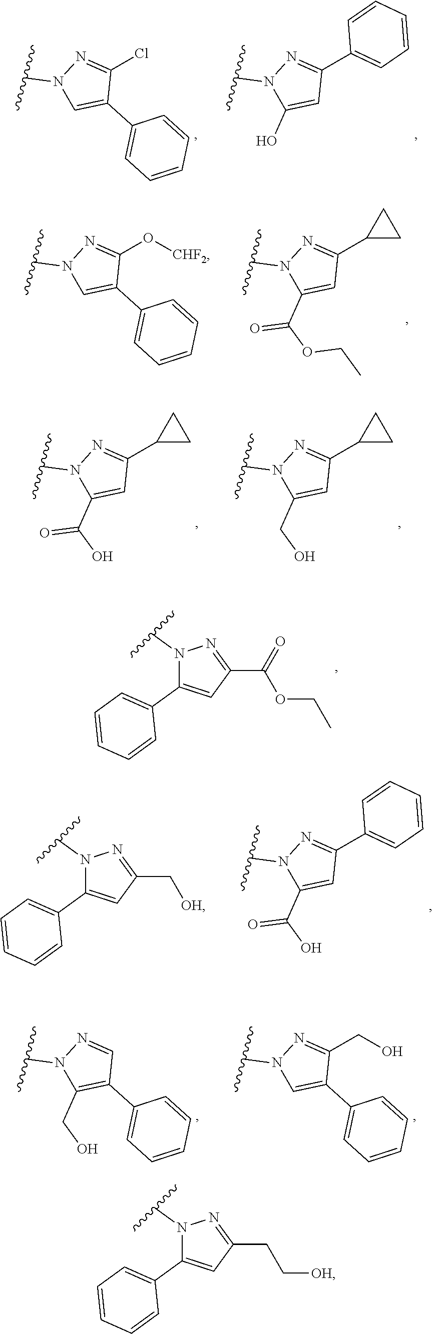

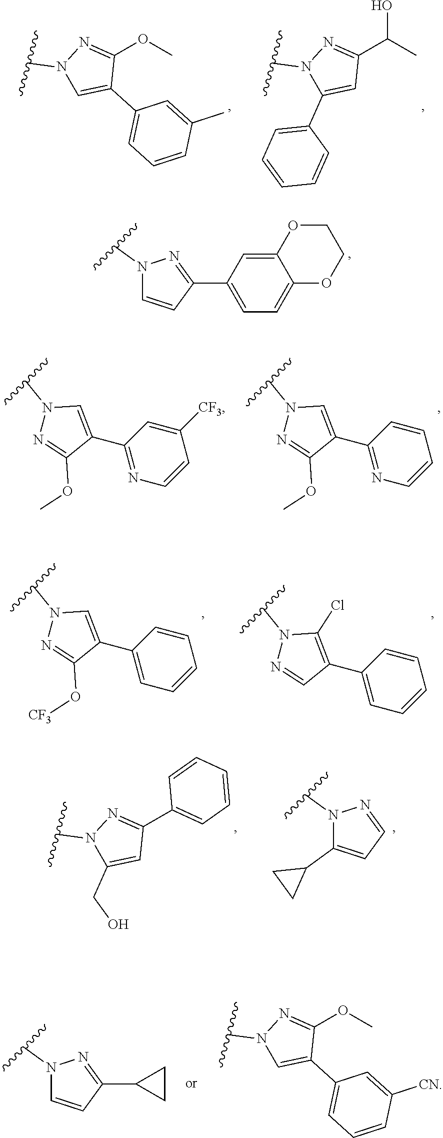

In some embodiments, R¹ is optionally substituted pyrazol-3-yl. In some embodiments, R¹ is pyrazol-3-yl substituted at position 1. In some embodiments, R¹ is substituted with optionally substituted C₆-C₁₀ aryl, optionally substituted C₁₋₉ heterocyclyl, optionally substituted C₂-C₉ heteroaryl, or optionally substituted C₃₋₈ cycloalkyl. In some embodiments, R¹ is

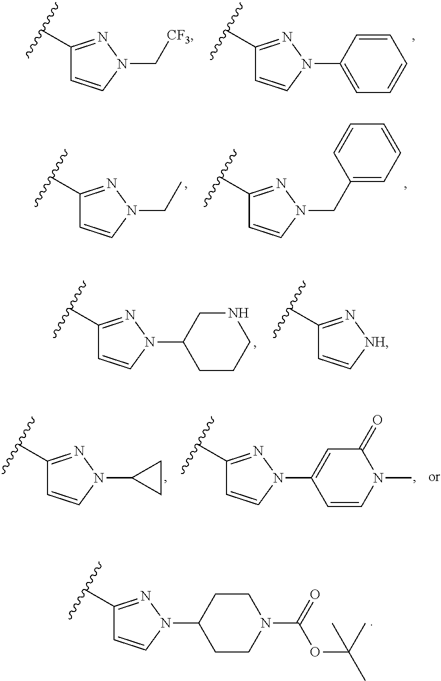

In some embodiments, R¹ is optionally substituted pyrimidin-6-yl. In some embodiments, R¹ is optionally substituted pyrimidin-4-yl. In some embodiments, R¹ is

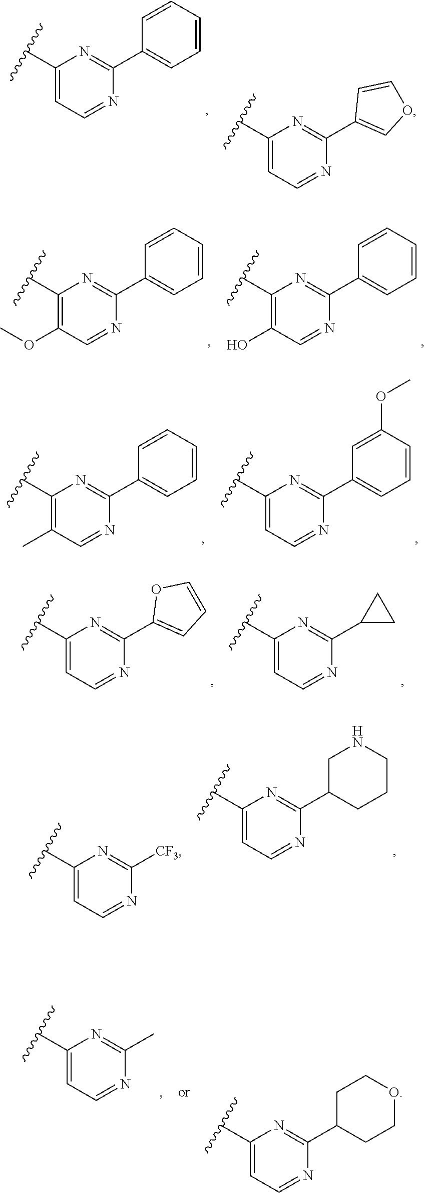

In some embodiments, R¹ is phenyl substituted with methoxy, optionally substituted C₁-C₆ alkyl, hydroxyl, optionally substituted C₂-C₉ heteroaryl, optionally substituted C₆-C₁₀ aryl, optionally substituted C₂-C₉ heterocyclyl, or C₃-C₈ cycloalkoxy. In some embodiments, R¹ is substituted with C₂-C₉ heteroaryl.

In some embodiments, R¹ is

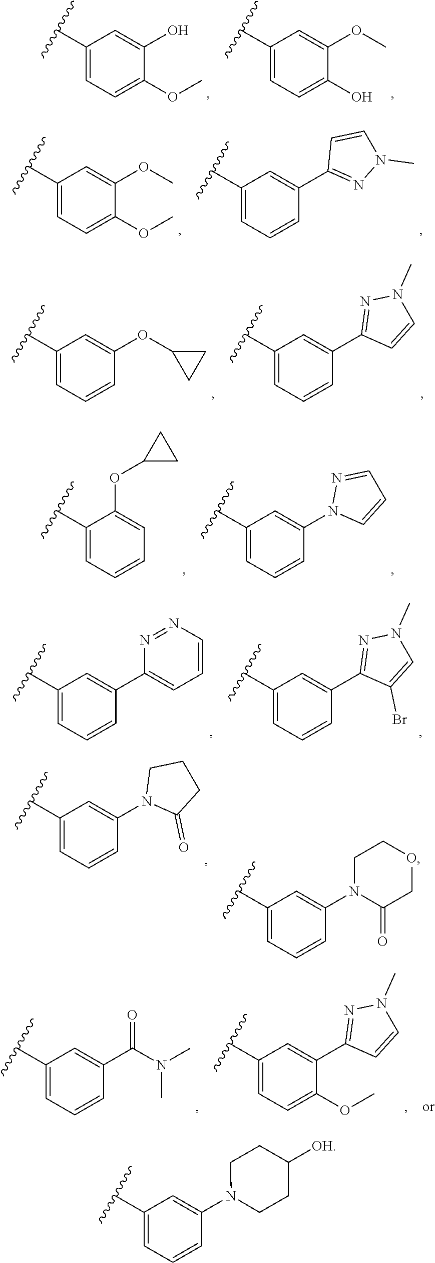

In some embodiments, R² is optionally substituted C₂-C₉ heteroaryl. In some embodiments, R² is optionally substituted pyridyl. In some embodiments, R² is pyridin-4-yl. In some embodiments, R² is optionally substituted tetrahydropyranyl, optionally substituted dihydropyranyl, optionally substituted piperidinyl, or optionally substituted azetidinyl. In some embodiments, R² is optionally substituted tetrahydropyran-4-yl, optionally substituted 5,6-dihydro-2H-pyran-4-yl, optionally substituted piperidin-4-yl, or optionally substituted piperidin-3-yl.

In some embodiments, R^{1A} is substituted with oxo.

In some embodiments, the compound has the structure:

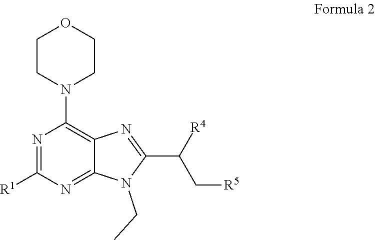

or a pharmaceutically acceptable salt thereof,
where R¹ is optionally substituted pyrazol-1-yl, phenyl substituted with optionally substituted C₂-C₉ heteroaryl, or optionally substituted pyridimin-4-yl; and
R⁴ and R⁵ are each, independently, hydroxyl or methoxy.

In some embodiments, R⁴ and R⁵ are hydroxyl. In some embodiments, R⁴ and R⁵ are methoxy. In some embodiments, R⁴ is hydroxyl and R⁵ is methoxy. In some embodiments, R⁴ is methoxy and R⁵ is hydroxyl. In some embodiments, R¹ is optionally substituted pyrazol-1-yl. In some embodiments, where R¹ is

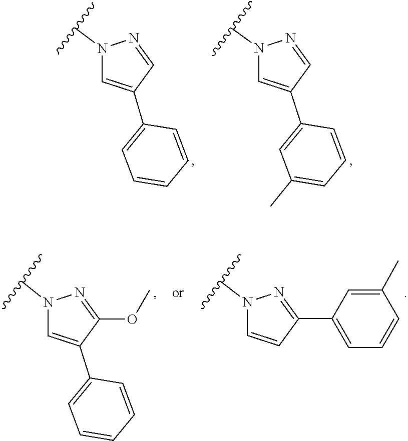 [SMILES: *n1cc(-c2ccccc2)cn1 ; *n1cc(-c2cccc(C)c2)cn1 ; *n1cc(-c2ccccc2)c(OC)n1 ; *n1ccc(-c2cccc(C)c2)n1]

In some embodiments, R¹ is phenyl substituted with optionally substituted C₂-C₉ heteroaryl. In some embodiments, R¹ is

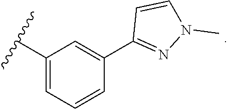 [SMILES: *c1cccc(-c2ccn(C)n2)c1]

In some embodiments, R¹ is optionally substituted pyridimin-4-yl. In some embodiments, R¹ is

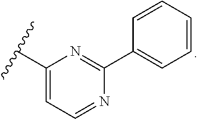 [SMILES: *c1ccnc(-c2ccccc2)n1]

In some embodiments, the compound has the structure:

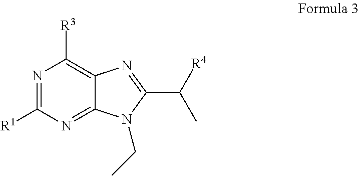

or a pharmaceutically acceptable salt thereof,
where R¹ is optionally substituted pyrazol-1-yl, phenyl substituted with optionally substituted heteroaryl, optionally substituted indazol-1-yl, or optionally substituted indazol-2-yl;
R⁴ is hydroxyl, 4-pyridinon-1-yl, —O-pyridin-3-yl, or CH₂OH; and
R³ is pyridin-4-yl or morpholin-1-yl.

In some embodiments, R⁴ is hydroxyl. In some embodiments, R⁴ is 4-pyridinon-1-yl. In some embodiments, R⁴ is —O-pyridin-3-yl. In some embodiments, R⁴ is CH₂OH. In some embodiments, R³ is pyridin-4-yl. In some embodiments, R³ is morpholin-1-yl. In some embodiments, R¹ is optionally substituted pyrazol-1-yl. In some embodiments, R¹ is

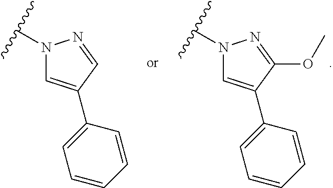 [SMILES: *n1cc(-c2ccccc2)cn1 ; *n1cc(-c2ccccc2)c(OC)n1]

In some embodiments, R¹ is phenyl substituted with optionally substituted heteroaryl. In some embodiments, R¹ is

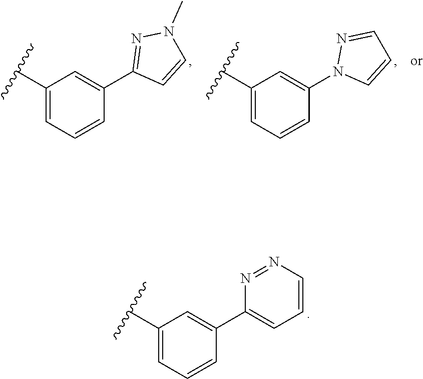

In some embodiments, R¹ is optionally substituted indazol-1-yl. In some embodiments, R¹ is

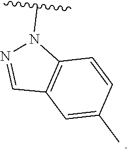 [SMILES: *n1ncc2cc(C)ccc21]

In some embodiments, R¹ is optionally substituted indazol-2-yl. In some embodiments, R¹ is

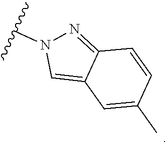 [SMILES: *n1cc2cc(C)ccc2n1]

In some embodiments, the compound has the structure:

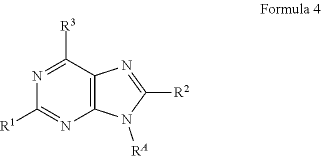

or a pharmaceutically acceptable salt thereof,
where R¹ is phenyl optionally substituted with methoxy or optionally substituted heteroaryl or optionally substituted pyrazol-1-yl,
R³ is morpholin-1-yl or piperidin-1-yl; and
R² is

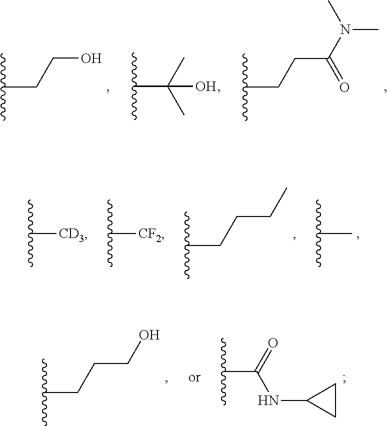

and
R^{A} is ethyl, 2-hydroxy-ethyl, or

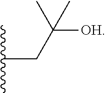 [SMILES: *CC(C)(C)O]

In some embodiments, R³ is morpholin-1-yl. In some embodiments, R³ is piperidin-1-yl. In some embodiments, R¹ is phenyl optionally substituted with methoxy or optionally substituted heteroaryl. In some embodiments, R¹ is

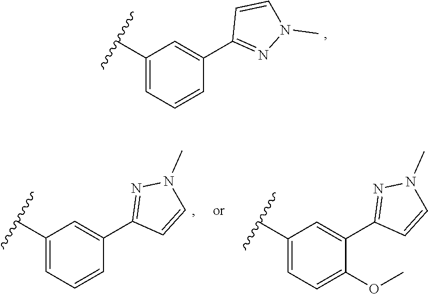

In some embodiments, R¹ is optionally substituted pyrazol-1-yl. In some embodiments, R¹ is

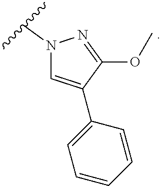 [SMILES: *n1cc(-c2ccccc2)c(OC)n1]

In some embodiments, the compound has the structure:

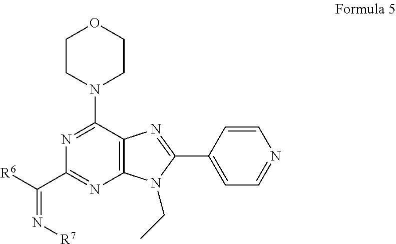

or a pharmaceutically acceptable salt thereof,
where R⁶ is hydrogen or methyl; and
R⁷ is optionally substituted phenoxy, optionally substituted benzyloxy, or optionally substituted amine.

In some embodiments, R⁶ is hydrogen. In some embodiments, R⁶ is methyl. In some embodiments, R⁷ is optionally substituted phenoxy. In some embodiments, R⁷ is

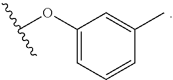 [SMILES: *Oc1cccc(C)c1]

In some embodiments, R⁷ is optionally substituted benzyloxy. In some embodiments, R⁷ is

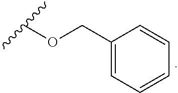 [SMILES: *OCc1ccccc1]

In some embodiments, R⁷ is optionally substituted amine. In some embodiments, R⁷ is

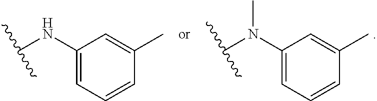

In some embodiments, the compound has the structure:

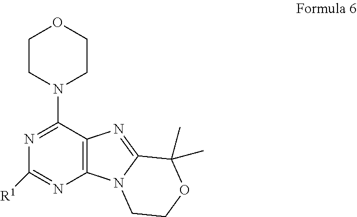

or a pharmaceutically acceptable salt thereof,
where R¹ is optionally substituted pyrazol-1-yl or —N(R^{1A})N═C(R^{1B})₂.

In some embodiments, R¹ is optionally substituted pyrazol-1-yl. In some embodiments, R¹ is

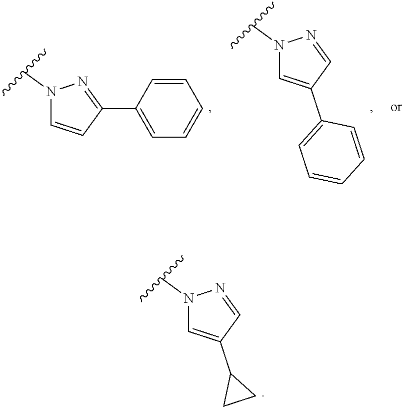 [SMILES: *n1ccc(-c2ccccc2)n1 ; *n1cc(-c2ccccc2)cn1 ; *n1cc(C2CC2)cn1]

In some embodiments, R¹ is —N(R^{1A})N═C(R^{1B})₂. In some embodiments, R¹ is

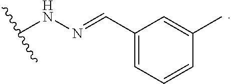 [SMILES: *N/N=C/c1cccc(C)c1]

In some embodiments, the compound has the structure:

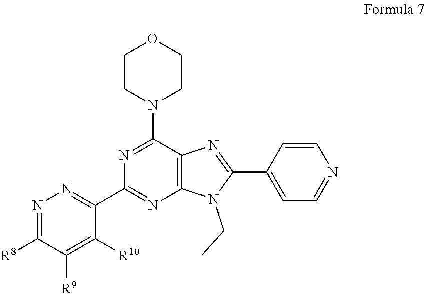

or a pharmaceutically acceptable salt thereof,
where R⁸ is hydrogen or methoxy;
R⁹ is hydrogen or phenyl; and
R¹⁰ is hydrogen or phenyl.

In some embodiments, R⁸ is hydrogen. In some embodiments, R⁸ is methoxy. In some embodiments, R⁹ is hydrogen. In some embodiments, R⁹ is phenyl. In some embodiments, R¹⁰ is hydrogen. In some embodiments, R¹⁰ is phenyl.

In some embodiments, the compound has the structure:

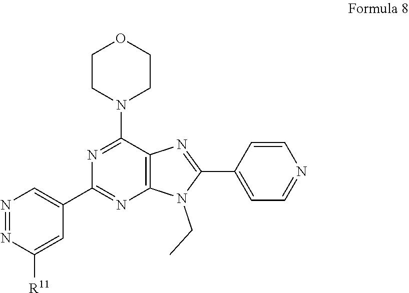

or a pharmaceutically acceptable salt thereof,
where R¹¹ is hydrogen or phenyl.

In some embodiments, R¹¹ is hydrogen. In some embodiments, R¹¹ is phenyl.

In some embodiments, the compound has the structure:

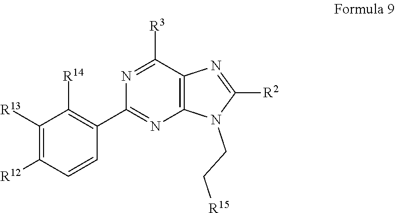

or a pharmaceutically acceptable salt thereof,
where R¹² is hydrogen, methoxy, or CH₂OH;
R¹³ is hydrogen, methoxy, C₃ cycloalkoxy, optionally substituted C₂-C₉ heteroaryl, optionally substituted C₂-C₉ heterocyclyl, or optionally substituted C₁-C₆ alkyl;
R¹⁴ is hydrogen or C₃ cycloalkoxy, or optionally substituted C₂-C₉ heterolaryl;
R¹⁵ is hydrogen or hydroxyl;
R² is hydrogen, pyridin-4-yl,

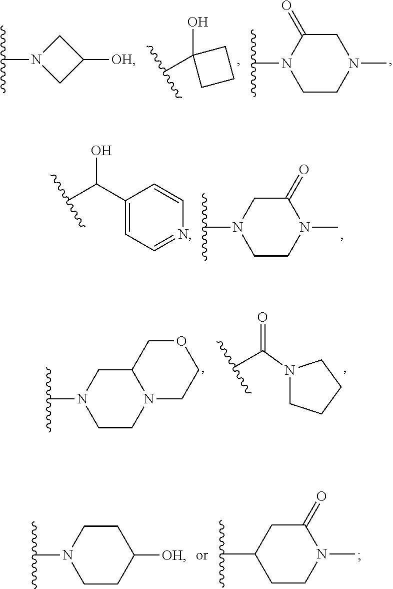

and
R³ is

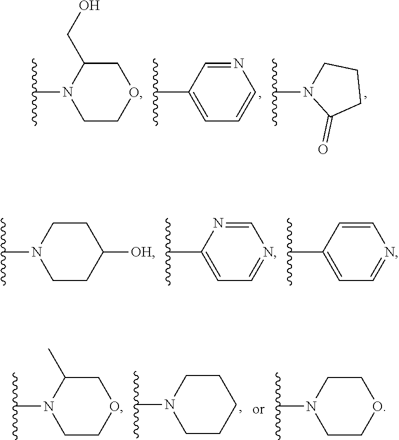

In some embodiments, R¹⁵ is hydrogen. In some embodiments, R¹⁵ is hydroxyl. In some embodiments, R¹² is hydrogen. In some embodiments, R¹² is methoxy. In some embodiments, R¹² is CH₂OH. In some embodiments, R¹⁴ is hydrogen. In some embodiments, R¹⁴ is C₃ cycloalkoxy. In some embodiments, R¹³ is hydrogen. In some embodiments, R¹³ is methoxy. In some embodiments, R¹³ is C₃ cycloaklkoxy. In some embodiments, R¹³ is optionally substituted C₂-C₉ heteroaryl. In some embodiments, R¹³ is pyrazol-1-yl, 1-methyl-pyrazol-3-yl, pyridazin-3-yl, or 4-bromo-1-methyl-pyrazol-3-yl. In some embodiments, R¹³ is optionally substituted C₂-C₉ heterocyclyl. In some embodiments, R¹³ is

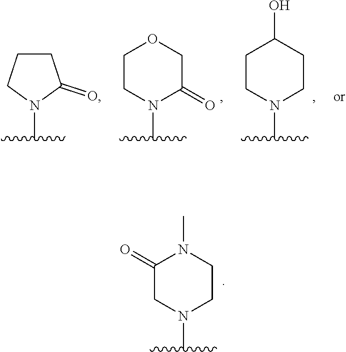

In some embodiments, R¹³ is optionally substituted C₁-C₆ alkyl. In some embodiments, R¹³ is CH₂OH or

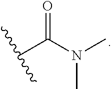 [SMILES: *C(=O)N(C)C]

In some embodiments, the compound has the structure:

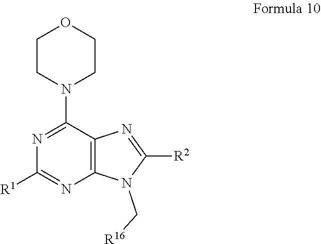

or a pharmaceutically acceptable salt thereof,
where R¹ is

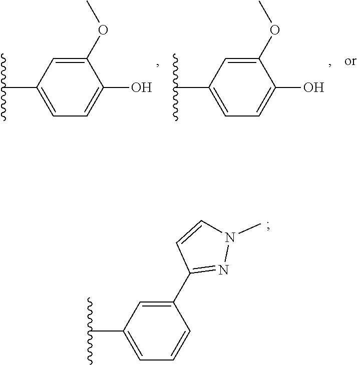

R¹⁶ is hydrogen or pyridine-3-yl; and
R² is pyridin-4-yl or hydrogen.

In some embodiments, R¹⁶ is hydrogen. In some embodiments, R¹⁶ is pyridine-3-yl. In some embodiments, R² is pyridin-4-yl. In some embodiments, R² is hydrogen.

In some embodiments, the compound has the structure:

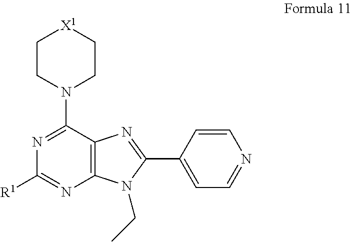

or a pharmaceutically acceptable salt thereof,
where X¹ is O or CH₂; and
R¹ is —N(R^{1A})N═C(R^{1B})₂.

In some embodiments, R¹ is

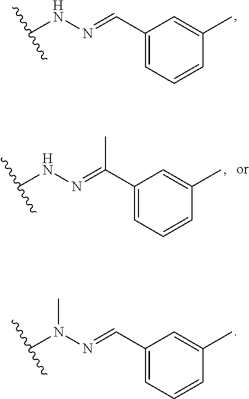 [SMILES: *N/N=C/c1cccc(C)c1 ; *N/N=C(\C)c1cccc(C)c1 ; *N(C)/N=C/c1cccc(C)c1]

In some embodiments, the compound has the structure:

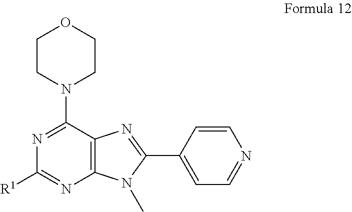

or a pharmaceutically acceptable salt thereof,

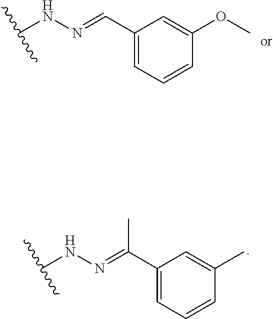 [SMILES: *N/N=C/c1cccc(OC)c1 ; *N/N=C(\C)c1cccc(C)c1]

where R¹ is

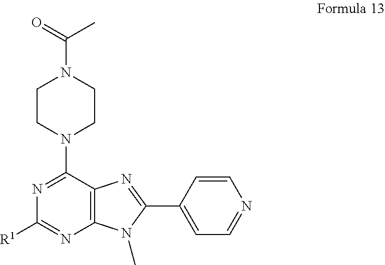

In some embodiments, the compound has the structure:
or a pharmaceutically acceptable salt thereof,
where R¹ is —N(R^{1A})N═C(R^{1B})₂.

In some embodiments, R¹ is

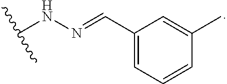 [SMILES: *N/N=C/c1cccc(C)c1]

In some embodiments, the compound has the structure:

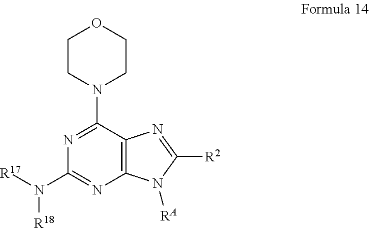

or a pharmaceutically acceptable salt thereof,
where R¹⁷ is optionally substituted C₆-C₁₀ aryl C₁-C₆ alkyl; optionally substituted C₆-C₁₀ heteroaryl C₁-C₆ alkyl; —NH₂, optionally substituted C₃-C₈ cycloalkyl; or optionally substituted C₂-C₉ heterlaryl;
R¹⁸ is hydrogen or optionally substituted C₁-C₆ alkyl;
R^{A} is methyl or ethyl; and
R² is pyridin-4-yl or hydrogen.

In some embodiments, R¹⁸ is hydrogen. In some embodiments, R¹⁸ is optionally substituted C₁-C₆ alkyl. In some embodiments, R¹⁸ is methyl. In some embodiments, R¹⁸ is ethyl. In some embodiments, R^{A} is methyl. In some embodiments, R^{A} is ethyl. In some embodiments, R² is pyridine-4-yl. In some embodiments, R² is hydrogen. In some embodiments, R¹⁷ is optionally substituted C₆-C₁₀ aryl C₁-C₆ alkyl. In some embodiments, R¹⁷ is

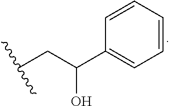 [SMILES: *CC(O)c1ccccc1]

In some embodiments, R¹⁷ is optionally substituted C₆-C₁₀ heteroaryl C₁-C₆ alkyl. In some embodiments, R¹⁷ is

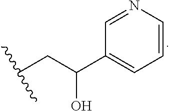 [SMILES: *CC(O)c1cccnc1]

In some embodiments, R¹⁷ is —NH2. In some embodiments, R¹⁷ is optionally substituted C₃-C₈ cycloalkyl. In some embodiments, R¹⁷ is

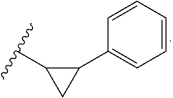 [SMILES: *C1CC1c1ccccc1]

In some embodiments, R¹⁷ is optionally substituted C₂-C₉ heteroaryl. In some embodiments, R¹⁷ is

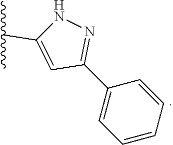 [SMILES: *c1cc(-c2ccccc2)n[nH]1]

In some embodiments, the compound has the structure:

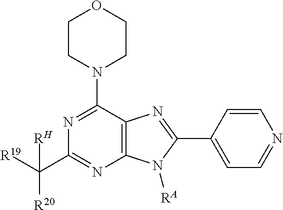

or a pharmaceutically acceptable salt thereof,
where R¹⁹ is optionally substituted amino, optionally substituted C₂-C₉ heterocycle, optionally substituted C₂-C₉ heteroaryl;
R^{H} and R²⁰, together with the atom to which they are attached, combine to form oxo;
R²⁰ is hydrogen or R²⁰ and R^{H}, together with the atom to which they are attached, combine to form oxo; and
R^{A} is ethyl or cyclopropyl.

In some embodiments, R^{A} is ethyl. In some embodiments, R^{A} is cyclopropyl. In some embodiments, R²⁰ is hydrogen. In some embodiments, R²⁰ and R^{H}, together with the atom to which they are attached, combine to form oxo. In some embodiments, R¹⁹ is optionally substituted amino. In some embodiments, R¹⁹ is

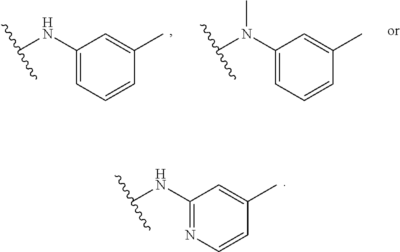

In some embodiments, R¹⁹ is optionally substituted C₂-C₉ heterocycle. In some embodiments, R¹⁹ is

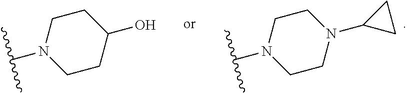 [SMILES: *N1CCC(O)CC1 ; "or" ; *N1CCN(C2CC2)CC1 ; "."]

In some embodiments, R¹⁹ is optionally substituted C₂-C₉ heteroaryl. In some embodiments, R¹⁹ is

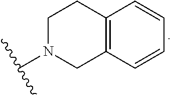 [SMILES: *N1CCc2ccccc2C1]

In some embodiments, R¹⁹ is optionally substituted C₆-C₁₀ aryl.

In some embodiments, the compound has the structure:

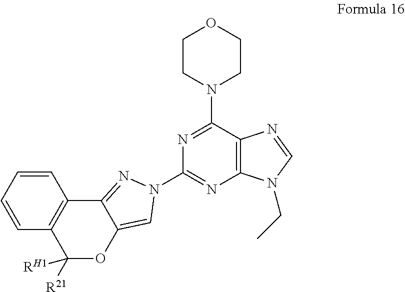

or a pharmaceutically acceptable salt thereof,
where R²¹ is hydrogen or R²¹ and R^{H}1, together with the atom to which they are attached, combine to form oxo; and
R^{H1} is hydrogen or R^{H1} and R²¹, together with the atom to which they are attached, combine to form oxo.

In some embodiments, where R²¹ and R^{H1}, together with the atom to which they are attached, combine to form oxo. In some embodiments, R²¹ is hydrogen.

In some embodiments, the compound has the structure:

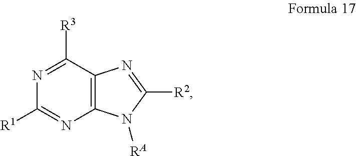

or a pharmaceutically acceptable salt thereof,
where R¹ is pyrazol-1-yl disubstituted with optionally substituted C₆-C₁₀ aryl; optionally substituted C₁-C₆ heteroalkyl; optionally substituted C₁-C₆ alkyl; optionally substituted C₂-C₉ heteroaryl, halo, hydroxy, optionally substituted C₃-C₈ cycloalkyl, or optionally substituted C₁-C₆ alkyl;
R³ is

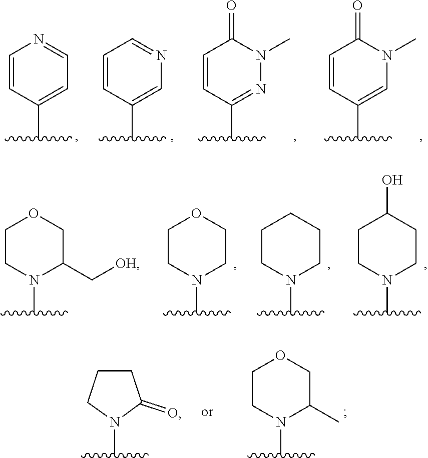

R^{A} is ethyl, 2-hydroxy-ethyl, methyl, or

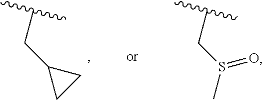

and R² is hydrogen, methyl, ethyl, halo, pyridin-3-yl, pyridin-4-yl, cyclopropyl,

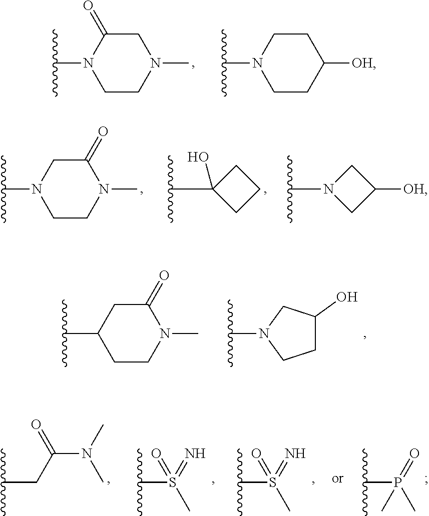

or R² and R^{A}, together with the atoms to which they are attached, combine to form an optionally substituted C₄ heterocyclyl.
R¹ is

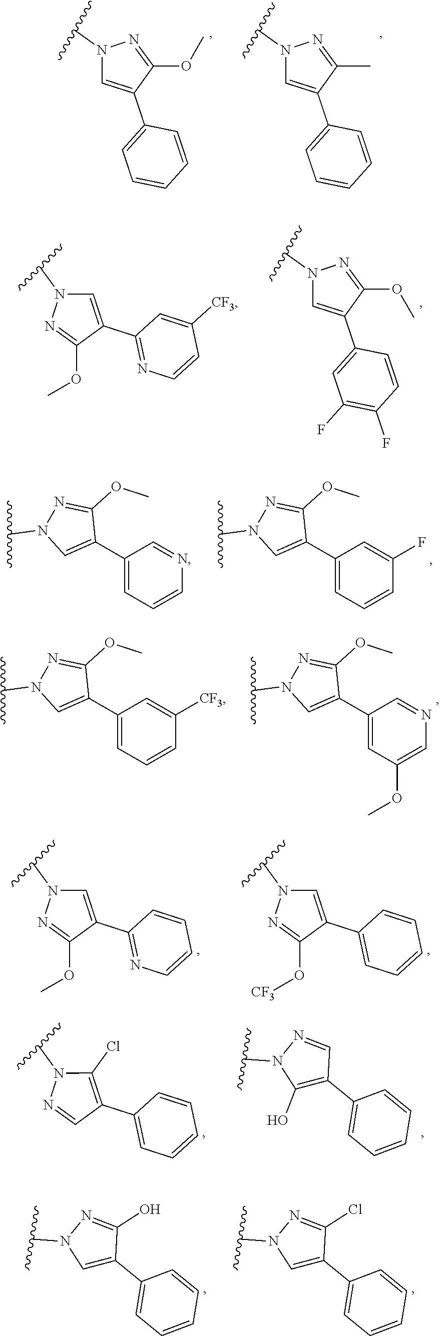

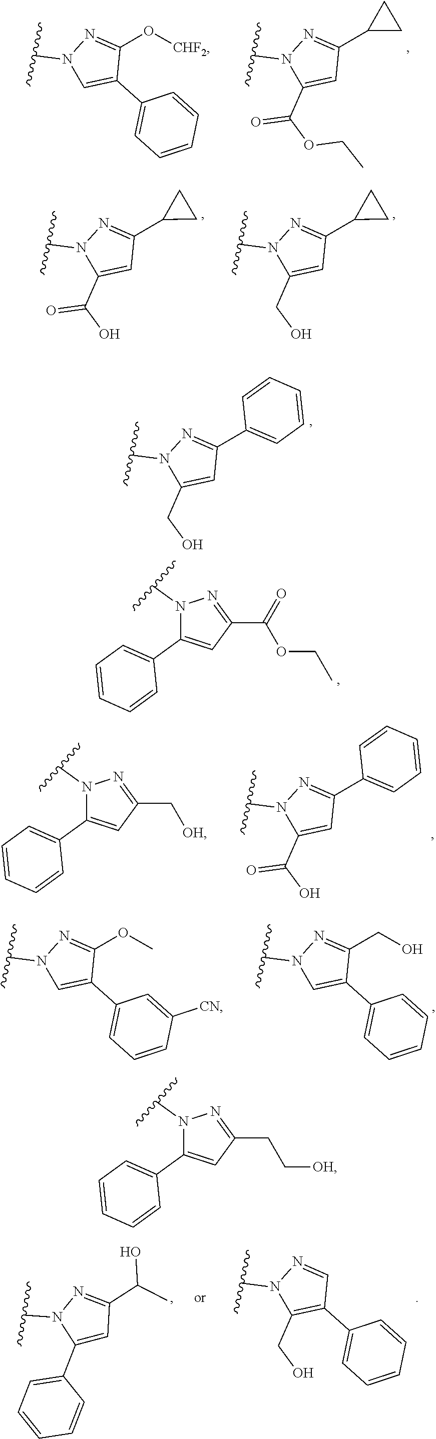

In some embodiments, the compound has the structure:

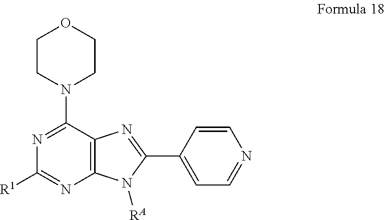

where R¹ is optionally substituted triazolyl; and
R^{A} is methyl, ethyl, or cyclopropyl.

In some embodiments, R^{A} is methyl. In some embodiments, R^{A} is ethyl. In some embodiments R^{A} is cyclopropyl. In some embodiments, R¹ is

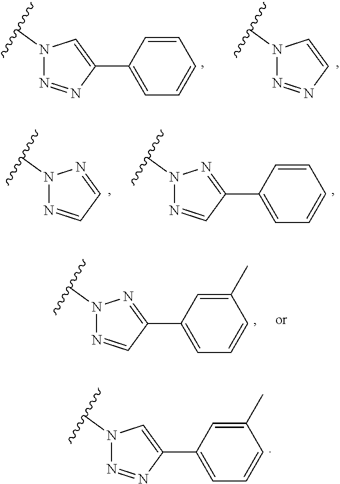 [SMILES: *n1cc(-c2ccccc2)nn1 ; *n1ccnn1 ; *n1nccn1 ; *n1ncc(-c2ccccc2)n1 ; *n1ncc(-c2cccc(C)c2)n1 ; *n1cc(-c2cccc(C)c2)nn1]

In some embodiments, the compound has the structure:

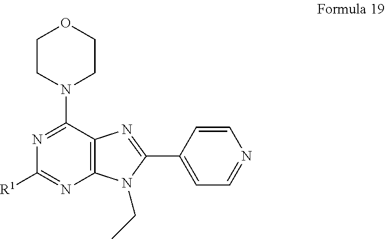

or a pharmaceutically acceptable salt thereof,
where R¹ is optionally substituted indazolyl or optionally substituted 4,5,6,7-tetrahydrotriazaindenyl.

In some embodiments, is optionally substituted indazolyl. In some embodiments, R¹ is

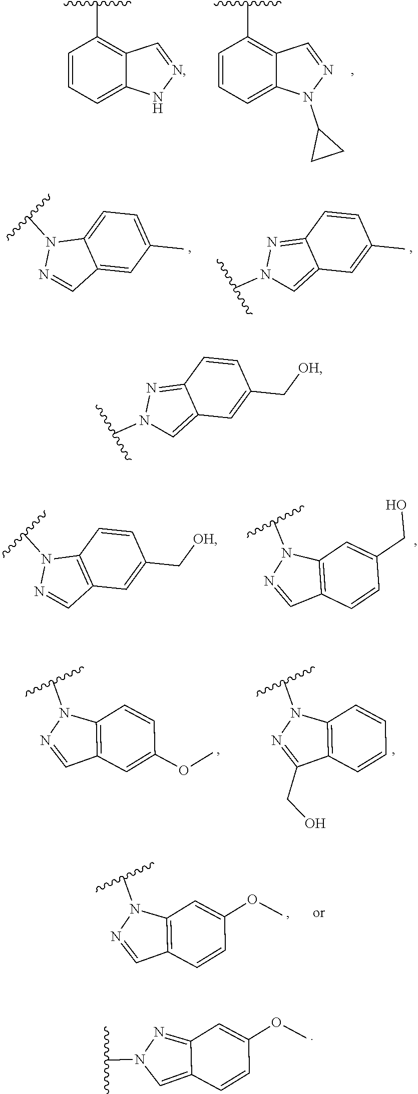

In some embodiments, R¹ is optionally substituted 4,5,6,7-tetrahydrotriazaindenyl. In some embodiments, R¹ is

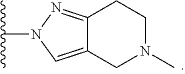 [SMILES: *n1cc2c(n1)CCN(C)C2]

In some embodiments, the compound has the structure:

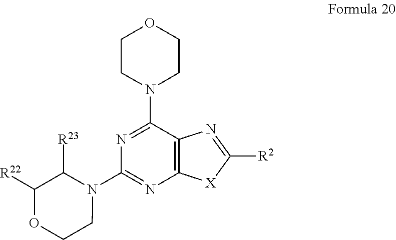

or a pharmaceutically acceptable salt thereof,
where X is S or NR^{A}.
R²² is hydrogen or phenyl;
R²³ is hydrogen or methyl;
R² is pyrazol-3-yl, pyridine-4-yl, or 4-phenyl-pyrazol-1-yl; and
R^{A} is methyl.

In some embodiments, X is S. In some embodiments, X is NR^{A}. In some embodiments, R²³ is hydrogen. In some embodiments, R²³ is methyl. In some embodiments, R² is pyrazol-3-yl. In some embodiments, R² is pyrazol-4-yl. In some embodiments, R² is pyridine-4-yl. In some embodiments, R² is 4-phenyl-pyrazol-1yl.

In some embodiments, the compound has the structure:

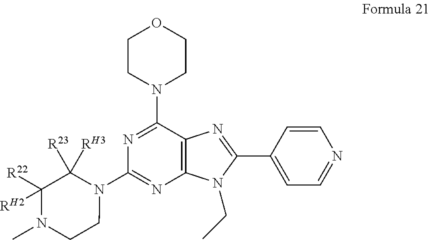

or a pharmaceutically acceptable salt thereof,
where R²² is phenyl, pyridine-2-yl, or R²² and R^{H2} together with the atom to which they are attached, combine to form oxo;
R^{H2} is hydrogen or R^{H2} and R²² together with the atom to which they are attached, combine to form oxo;
R²³ is hydrogen or R²³ and R^{H3}, together with the atom to which they are attached, combine to form oxo; and
R^{H3} is hydrogen or R^{H3} and R²³, together with the atom to which they are attached, combine to form oxo.

In some embodiments, R²³ is hydrogen. In some embodiments, R²³ and R^{H3}, together with the atom to which they are attached, combine to form oxo.

In some embodiments, the compound has the structure:

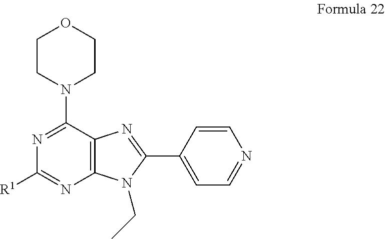

or a pharmaceutically acceptable salt thereof,
where R¹ is

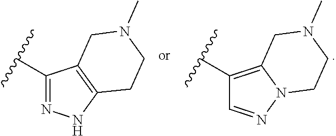 [SMILES: *c1n[nH]c2c1CN(C)CC2 ; *c1cnn2c1CN(C)CC2]

In some embodiments, the compound has the structure:

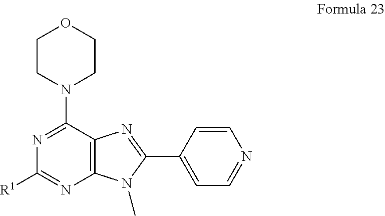

or a pharmaceutically acceptable salt thereof,
where R¹ is

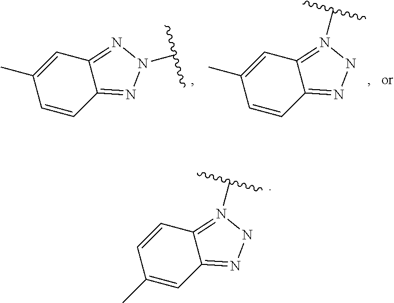

In some embodiments, the compound has the structure:

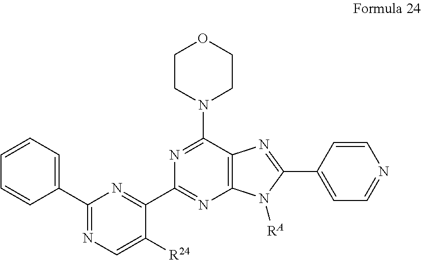

or a pharmaceutically acceptable salt thereof,
where R²⁴ is methoxy, methyl or hydroxyl; and
R^{A} is methyl or ethyl.

In some embodiments, R^{A} is methyl. In some embodiments, R^{A} is ethyl.

In some embodiments, the compound has the structure:

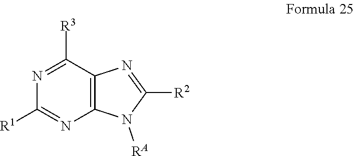

or a pharmaceutically acceptable salt thereof,
where R¹ is optionally substituted pyrazolyl, optionally substituted pyrimidin-3-yl, or optionally substituted pyridin-4-yl;
R^{A} is methyl or ethyl;
R² is optionally substituted C₂-C₉ heteroaryl, or optionally substituted C₁-C₉ heterocyclyl; and
R³ is

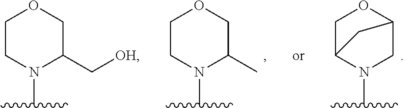

In some embodiments, R^{A} is methyl. In some embodiments, R^{A} is ethyl. In some embodiments, R¹ is optionally substituted pyrazolyl. In some embodiments, R¹ is

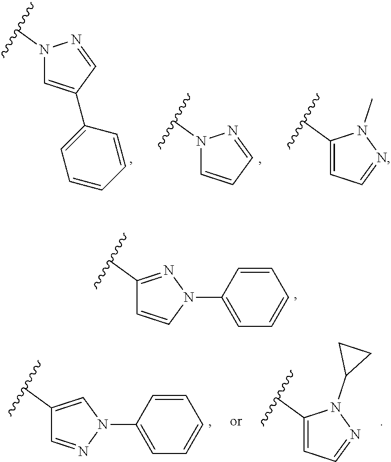

In some embodiments, R¹ is optionally substituted pyrimidin-4-yl. In some embodiments, R¹ is

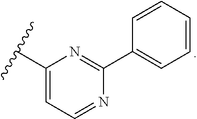 [SMILES: *c1ccnc(-c2ccccc2)n1]

In some embodiments, R¹ is optionally substituted pyridin-4-yl. In some embodiments, R¹ is

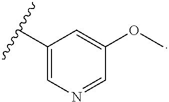 [SMILES: *c1cncc(OC)c1]

In some embodiments, R² is optionally substituted C₂-C₉ heteroaryl. In some embodiments, R² is pyridin-4-yl or 1-methyl-pyrazol-5-yl. In some embodiments, R² is optionally substituted C₁-C₉ heterocyclyl. In some embodiments, R² is

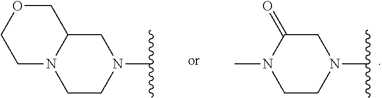

In some embodiments, the compound has the structure:

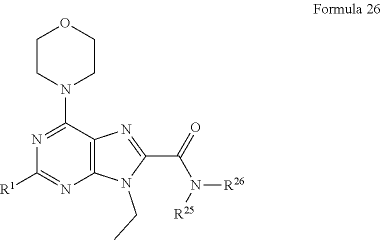

or a pharmaceutically acceptable salt thereof,
where R¹ is optionally substituted pyrazol-1-yl or phenyl substituted with optionally substituted C₂-C₉ heteroaryl; and
R²⁵ and R²⁶, together the atom to which they are attached, combine to form a C₃-C₅ heterocyclyl substituted with hydroxyl.

In some embodiments, R¹ is optionally substituted pyrazol-1-yl. In some embodiments, R¹ is

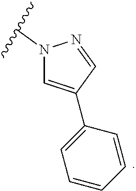 [SMILES: *n1cc(-c2ccccc2)cn1]

In some embodiments, R¹ is phenyl substituted with optionally substituted C₂-C₉ heteroaryl. In some embodiments, R¹ is

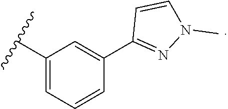 [SMILES: *c1cccc(-c2ccn(C)n2)c1]

In some embodiments, the heterocycle formed by the combination of R²⁵, R²⁶, and the atom to which they are attached is

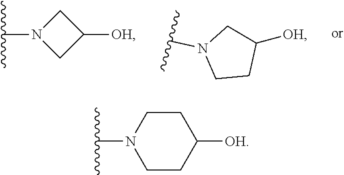 [SMILES: *N1CC(O)C1 ; *N1CCC(O)C1 ; "or" ; *N1CCC(O)CC1]

In some embodiments, the compound has the structure:

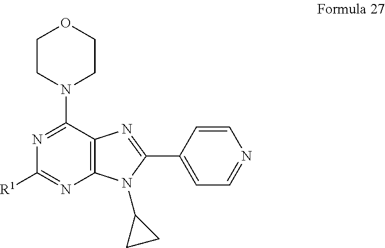

or a pharmaceutically acceptable salt thereof,
where R¹ is optionally substituted pyrazol-1-yl, optionally substituted pyrazol-5-yl, or phenyl substituted with methoxy or C₃-C₈ cycloalkoxy.

In some embodiments, R¹ is optionally substituted pyrazol-1-yl. In some embodiments, R¹ is optionally substituted pyrazol-5-yl. In some embodiments, R¹ is

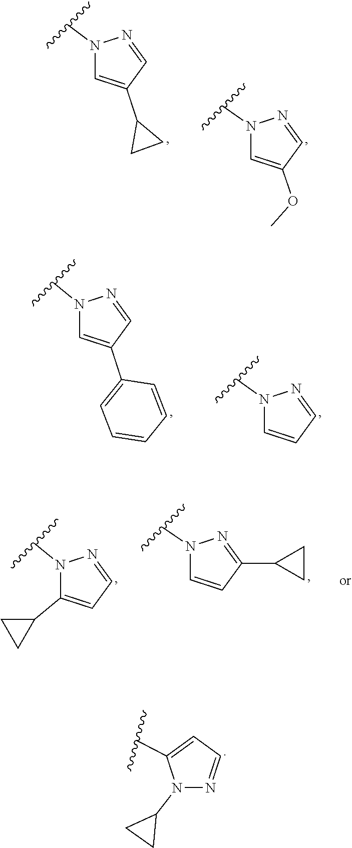

In some embodiments, R¹ is phenyl substituted with methoxy or C₃-C₈ cycloalkoxy. In some embodiments, R¹ is

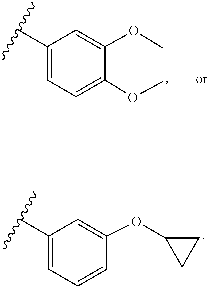 [SMILES: *c1ccc(OC)c(OC)c1 ; *c1cccc(OC2CC2)c1]

In some embodiments, the compound has the structure:

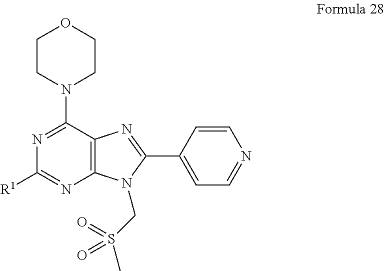

or a pharmaceutically acceptable salt thereof,
where R¹ is optionally substituted pyrazol-1-yl.

In some embodiments, R¹ is

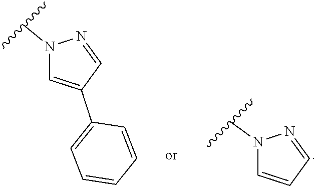 [SMILES: *n1cc(-c2ccccc2)cn1 ; *n1cccn1]

In some embodiments, the compound has the structure:

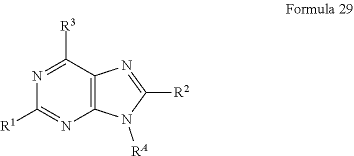

or a pharmaceutically acceptable salt thereof,
where R¹ is optionally substituted pyrazol-1-yl, optionally substituted pyrazol-3-yl, or optionally substituted pyrazol-5-yl;
R³ is morpholin-1-yl or piperidin-1-yl;
R^{A} is methyl or ethyl; and
R² is

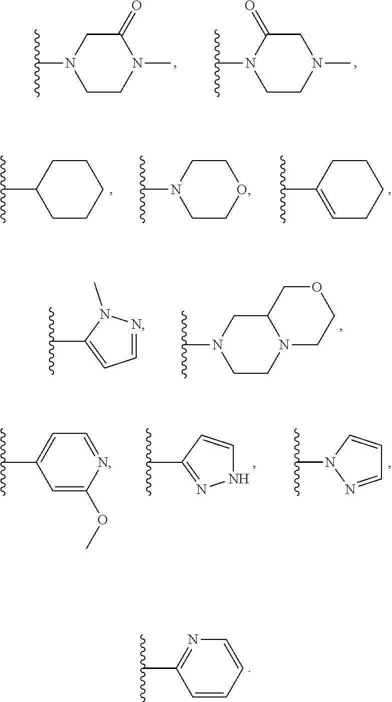

In some embodiments, R^{A} is methyl. In some embodiments, R^{A} is ethyl. In some embodiments where R¹ is optionally substituted pyrazol-1-yl. In some embodiments, R¹ is

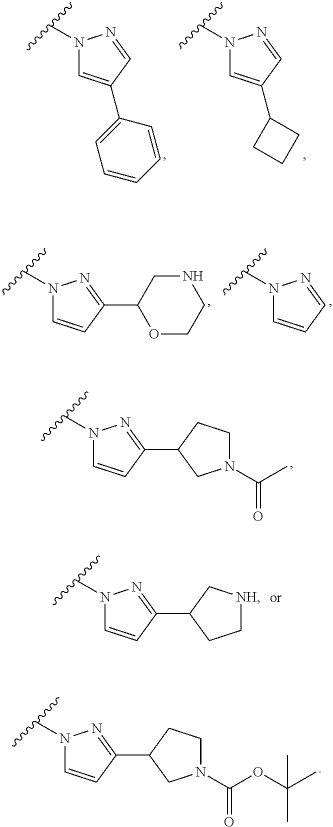

In some embodiments, R¹ is optionally substituted pyrazol-3-yl. In some embodiments, R¹ is

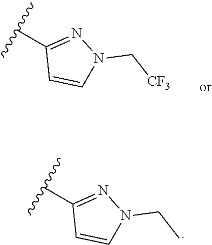

In some embodiments, R¹ is optionally substituted pyrazol-5-yl. In some embodiments, R¹ is

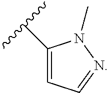 [SMILES: *c1ccnn1C]

In some embodiments, the compound has the structure:

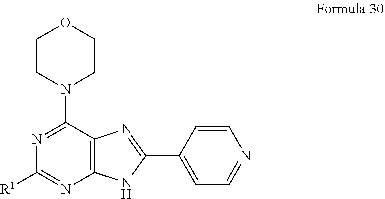

or a pharmaceutically acceptable salt thereof,
where R¹ is pyrazolyl monosubstituted with optionally substituted C₂-C₉ heterocyclyl or C₆-C₁₀ aryl.

In some embodiments, R¹ is

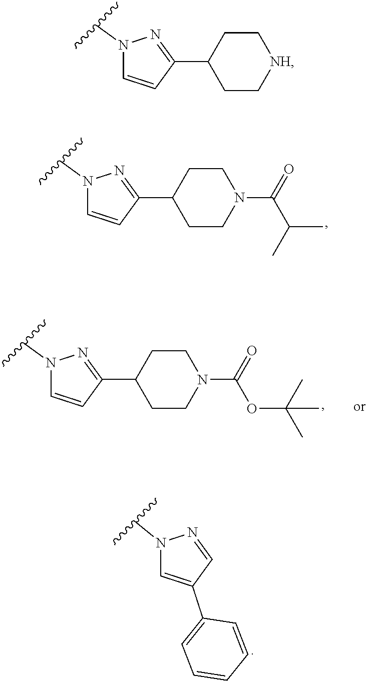 [SMILES: *n1ccc(C2CCNCC2)n1 ; *n1ccc(C2CCN(C(=O)C(C)C)CC2)n1 ; *n1ccc(C2CCN(C(=O)OC(C)(C)C)CC2)n1 ; *n1cc(-c2ccccc2)cn1]

In some embodiments, the compound has the structure:

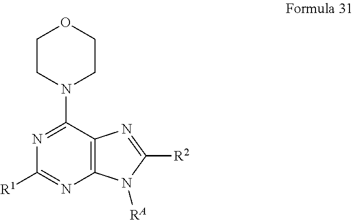

or a pharmaceutically acceptable salt thereof,
where R¹ is optionally substituted pyrazol-1-yl or optionally substituted pyrimidin-4-yl optionally substituted with optionally substituted C₁-C₆ alkyl;
R^{A} is methyl or difluoromethyl;
R² is pyridin-4-yl or

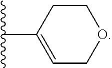 [SMILES: *C1=CCOCC1]

In some embodiments, R^{A} is methyl. In some embodiments, R^{A} is difluoromethyl. In some embodiments, R¹ is optionally substituted pyrazol-1-yl. In some embodiments, R¹ is

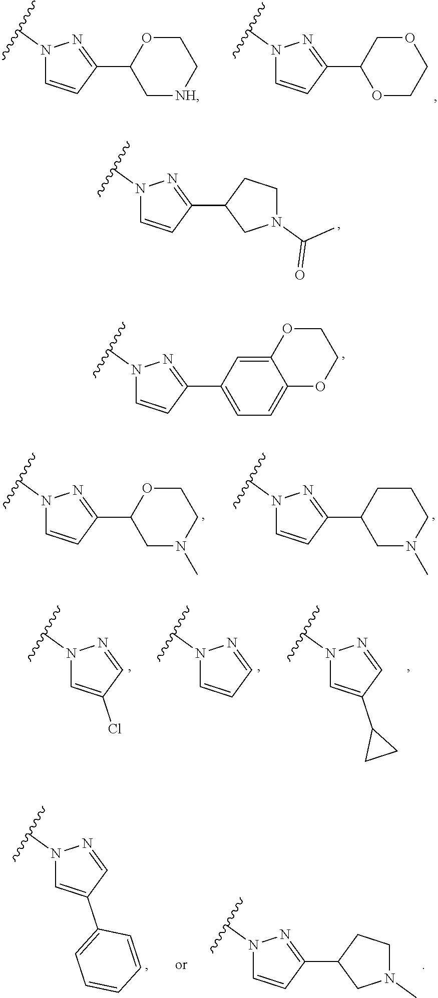

In some embodiments, R¹ is optionally substituted pyrimidin-4-yl. In some embodiments, R¹ is

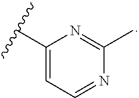 [SMILES: *c1ccnc(C)n1]

In some embodiments, the compound has the structure:

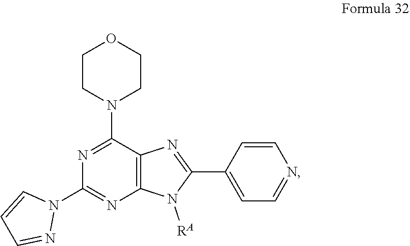

or a pharmaceutically acceptable salt thereof,
where R^{A} is

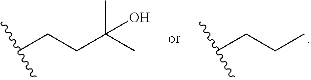

In some embodiments, the compound has the structure:

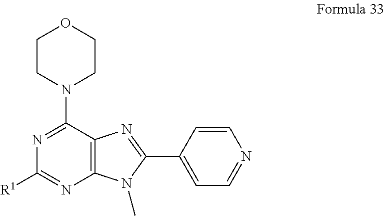

or a pharmaceutically acceptable salt thereof,
where R¹ is

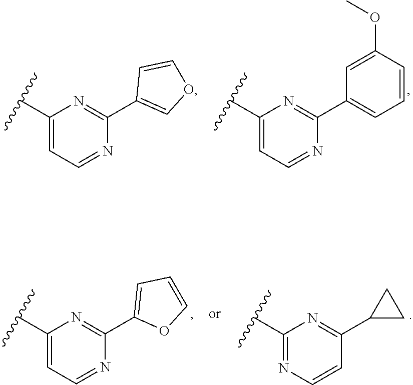

In some embodiments, the compound has the structure:

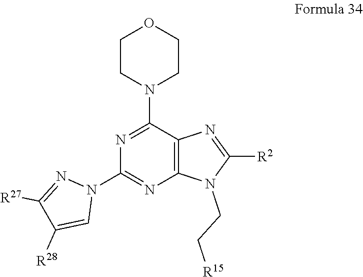

or a pharmaceutically acceptable salt thereof,
where R²⁷ is hydrogen, tetrahydropyran-3-yl, or tetrahydropyran-4-yl;
R²⁸ is hydrogen, methoxy, phenyl, methyl, difluoromethyl, optionally substituted cyclobutyl,
R¹⁵ is hydrogen or methoxy; and
R² is pyridin-4-yl or —O-pyridin-4-yl.

In some embodiments, R¹⁵ is hydrogen. In some embodiments, R¹⁵ is methoxy. In some embodiments, R² is pyridine-4-yl. In some embodiments, R² is —O-pyridin-4-yl.

In some embodiments, the compound has the structure:

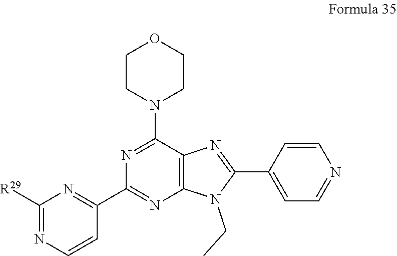

or a pharmaceutically acceptable salt thereof,
where R²⁹ is optionally substituted C₂-C₉ heterocyclyl or optionally substituted C₆-C₁₀ aryl.

In some embodiments, R²⁹ is optionally substituted C₂-C₉ heterocyclyl. In some embodiments, R²⁹ is tetrohydropyran-4-yl. In some embodiments, R²⁹ is optionally substituted C₆-C₁₀ aryl. In some embodiments, R²⁹ is phenyl.

In some embodiments, the compound has the structure:

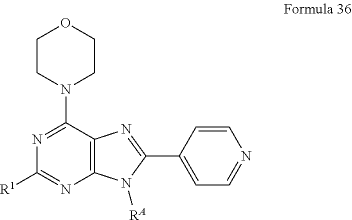

or a pharmaceutically acceptable salt thereof,
where R¹ is optionally substituted 4,5-dihydro-pyrazol-1-yl, optionally substituted imidazol-2-yl, optionally substituted piperidin-1-yl, or optionally substituted 1,2,4-triazol-3-yl, optionally substituted pyrazol-4-yl, optionally substituted 1,3,4-oxadiazol-2-yl, or optionally substituted pyridin-3-yl; and
R^{A} is methyl or ethyl.

In some embodiments, R^{A} is methyl. In some embodiments, R^{A} is ethyl. In some embodiments R¹ is optionally substituted 4,5-dihydro-pyrazol-1-yl. In some embodiments, R¹ is

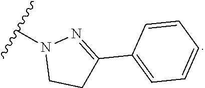 [SMILES: *N1CCC(c2ccccc2)=N1]

In some embodiments, R¹ is optionally substituted 1,2,3,4-tetrahydroquinolin-7-yl. In some embodiments, R¹ is

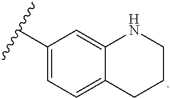 [SMILES: *c1ccc2c(c1)NCCC2]

In some embodiments, R¹ is optionally substituted imidazol-2-yl. In some embodiments, R¹ is

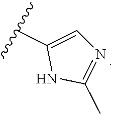 [SMILES: *c1cnc(C)[nH]1]

In some embodiments, R¹ is optionally substituted piperidin-1-yl. In some embodiments, R¹ is

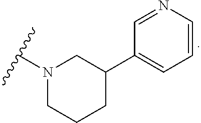 [SMILES: *N1CCCC(c2cccnc2)C1]

In some embodiments, R¹ is optionally substituted 1,2,4-triazol-3-yl. In some embodiments, R¹ is

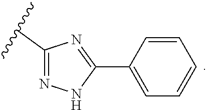 [SMILES: *c1n[nH]c(-c2ccccc2)n1]

In some embodiments, R¹ is optionally substituted pyrazol-4-yl. In some embodiments, R¹ is

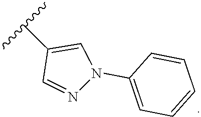 [SMILES: *c1cnn(-c2ccccc2)c1]

In some embodiments, R¹ is optionally substituted 1,3,4-oxadiazol-2-yl. In some embodiments, R¹ is

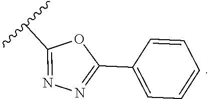 [SMILES: *c1nnc(-c2ccccc2)o1]

In some embodiments, R¹ is optionally substituted pyridin-3-yl. In some embodiments, R¹ is

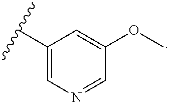 [SMILES: *c1cncc(OC)c1]

In some embodiments, the compound has the structure:

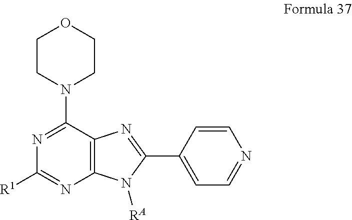

or a pharmaceutically acceptable salt thereof,
where R¹ is pyrazol-5-yl optionally substituted with C₂-C₉ heteroaryl, C₆-C₁₀ aryl, C₃-C₈ cycloalkyl or C₃-C₈ cycloalkyl C₁-C₆ alkyl; and
R^{A} is methyl or ethyl.

In some embodiments, R¹ is

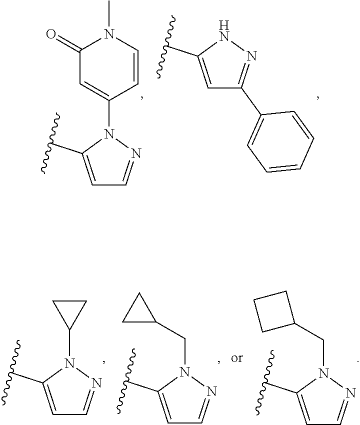

In some embodiments, the compound has the structure:

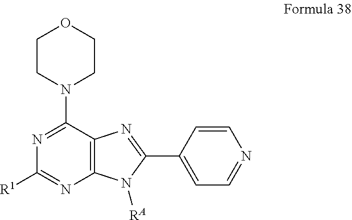

or a pharmaceutically acceptable salt thereof,
where R¹ is pyrazol-3-yl substituted with optionally substituted C₂-C₉ heteroaryl, optionally substituted C₃-C₈ cycloalkyl, optionally substituted C₂ alkyl, or optionally substituted C₆-C₁₀ aryl C₁-C₆ alkyl; and
R^{A} is methyl or ethyl.

In some embodiments, R^{A} is methyl. In some embodiments, R^{A} is ethyl. In some embodiments R¹ is

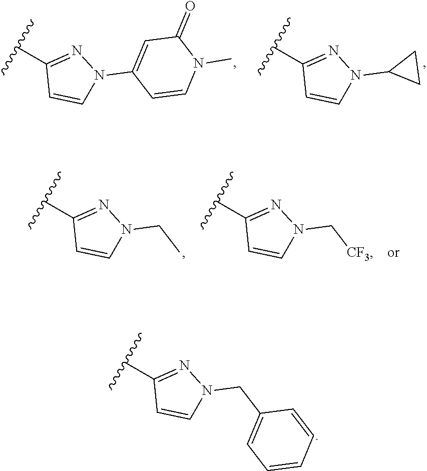

In some embodiments, the compound has the structure:

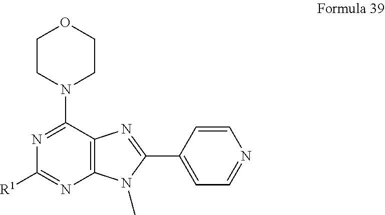

or a pharmaceutically acceptable salt thereof,
where R¹ is pyrazol-3-yl disubstituted with C₁-C₆ alkyl or C₆-C₁₀ aryl.

In some embodiments, R¹ is

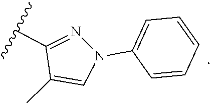 [SMILES: *c1nn(-c2ccccc2)cc1C]

In an aspect, the invention provides a compound of formula (40)

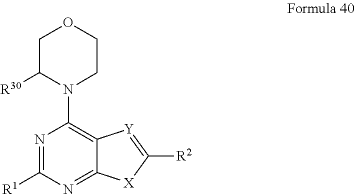

or a pharmaceutically acceptable salt thereof;
where Y is CH or N;
X is O, or S;
R¹ is optionally substituted morpholin-1-yl, optionally substituted pyrimidin-4-yl, —N(R^{1A})N═C(R^{1B})₂, optionally substituted pyrazol-3-yl, or optionally substituted indazol-4-yl;
R² is hydrogen or methyl; and
R³⁰ is optionally substituted pyridin-4-yl, optionally substituted pyrazol-3-yl, optionally substituted pyrazol-1-yl, or C₂-C₉ heterocycle C₁-C₆ alkyl substituted with —S(O)₂CH₃.

In some embodiments, Y is CH. In some embodiments, Y is N. In some embodiments, X is O. In some embodiments, X is S. In some embodiments, R² is hydrogen. In some embodiments, R² is methyl. In some embodiments, R¹ is optionally substituted morpholin-1-yl. In some embodiments, R¹ is

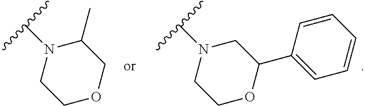

In some embodiments, R¹ is optionally substituted pyrimidin-4-yl. In some embodiments, R¹ is

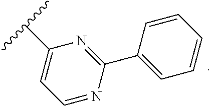 [SMILES: *c1ccnc(-c2ccccc2)n1]

In some embodiments, R¹ is —N(R^{1A})N═C(R^{1B})₂. In some embodiments, R¹ is

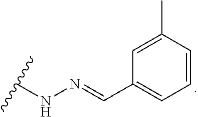 [SMILES: *N/N=C/c1cccc(C)c1]

In some embodiments, R¹ is optionally substituted pyrazol-3-yl. In some embodiments, R¹ is

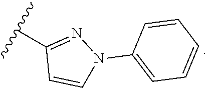 [SMILES: *c1ccn(-c2ccccc2)n1]

In some embodiments, R¹ is optionally substituted indazol-4-yl. In some embodiments, R¹ is

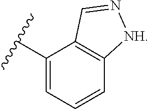 [SMILES: *c1cccc2[nH]ncc12]

In some embodiments, R³⁰ is optionally substituted pyridin-4-yl. In some embodiments, R³⁰ is pyridin-4-yl. In some embodiments, R³⁰ is optionally substituted pyrazol-3-yl. In some embodiments, R³⁰ is pyrazol-3-yl. In some embodiments, R³⁰ is optionally substituted pyrazol-1-yl. In some embodiments, R³⁰ is

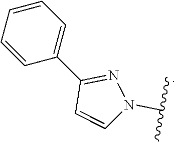 [SMILES: *n1ccc(-c2ccccc2)n1]

In some embodiments, R³⁰ is C₂-C₉ heterocycle C₁-C₆ alkyl substituted with —S(O)₂CH₃. In some embodiments, R³⁰ is

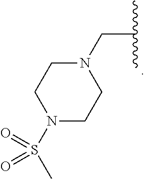 [SMILES: *CN1CCN(S(C)(=O)=O)CC1]

In an aspect, the invention provides a compound of formula (41)

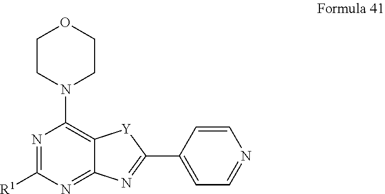

or a pharmaceutically acceptable salt thereof,
where Y is S or NR^{A}.
R¹ is optionally substituted pyrimidin-4-yl; and
R^{A} is optionally substituted C₁-C₆ alkyl.

In some embodiments, Y is S. In some embodiments, Y is N—CH₃. In some embodiments, R¹ is

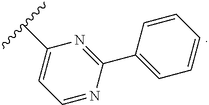 [SMILES: *c1ccnc(-c2ccccc2)n1]

In an aspect, the invention provides a compound of formula (42)

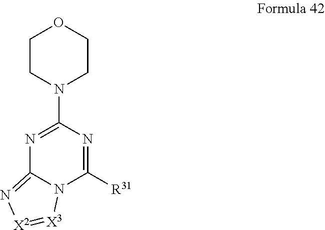

o a pharmaceutically acceptable salt thereof,
where X² and X³ are each, independently, N or CR³²
R³¹ is optionally substituted C₂-C₉ heteroaryl; and
R³² is optionally substituted C₂-C₉ heteroaryl.

In some embodiments, X² is N and X³ is CR³². In some embodiments, X² is CR³² and X³ is N. In some embodiments, R³¹ is optionally substituted pyraozl-1-yl. In some embodiments, R³¹ is

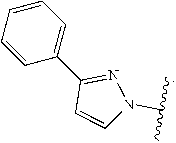 [SMILES: *n1ccc(-c2ccccc2)n1]

In some embodiments, R³² is optionally substituted pyridin-4-yl. In some embodiments, R³² is pyridin-4-yl.

In an aspect, the invention provides a compound of formula (43)

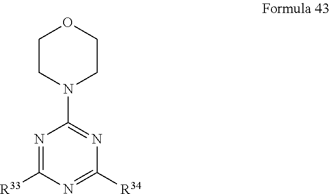

or a pharmaceutically acceptable salt thereof,
where R³³ is optionally substituted amino; and
R³⁴ is optionally substituted C₂-C₉ heteroaryl.

In some embodiments, R³³ is

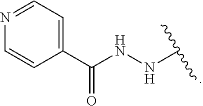 [SMILES: *NNC(=O)c1ccncc1]

In some embodiments, R³⁴ is optionally substituted pyrazol-1-yl. In some embodiments, R³⁴ is

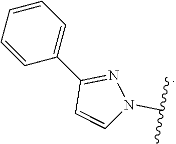 [SMILES: *n1ccc(-c2ccccc2)n1]

In an aspect, the invention provides a compound of formula (44)

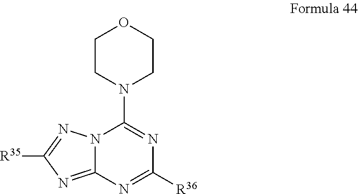

or a pharmaceutically acceptable salt thereof,
where R³⁵ and R³⁶ are each, independently, optionally substituted C₂-C₉ heteroaryl.

In some embodiments, R³⁵ is optionally substituted pyridine-4-yl. In some embodiments, R³⁵ is pyridine-4-yl. In some embodiments, R³⁶ is optionally substituted pyrazol-1-yl. In some embodiments, R³⁶ is

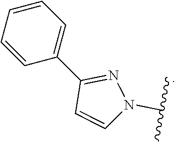 [SMILES: *n1ccc(-c2ccccc2)n1]

In an aspect, the invention provides a compound of formula (45)

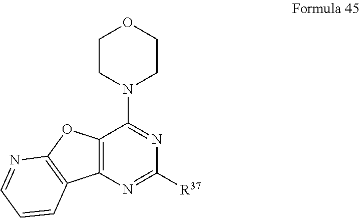

or a pharmaceutically acceptable salt thereof;
where R³⁷ is optionally substituted C₂-C₉ heteroaryl.

In some embodiments, R³⁷ is optionally substituted pyrazol-1-yl. In some embodiments, R³⁷ is

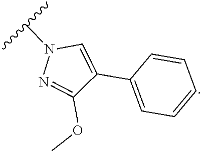 [SMILES: *n1cc(-c2ccccc2)c(OC)n1]

In an aspect, the invention provides a compound of formula (46)

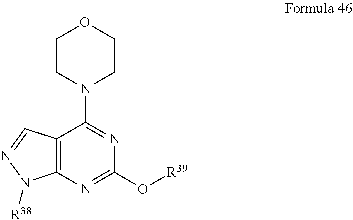

or a pharmaceutically acceptable salt thereof;
where R³⁸ is optionally substituted C₆-C₁₀ aryl; and
R³⁹ is optionally substituted C₂-C₉ heteroaryl C₁-C₆ alkyl.

In some embodiments, R³⁸ is phenyl. In some embodiments, R³⁹ is

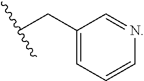 [SMILES: *Cc1cccnc1]

In some embodiments, the compound has the structure:

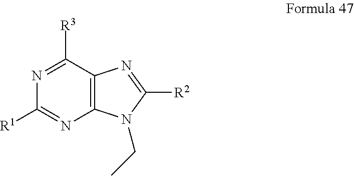

or a pharmaceutically acceptable salt thereof,
where R² is hydrogen, optionally substituted C₂-C₉ heteroaryl; optionally substituted C₂-C₉ heterocyclyl, or C₁-C₃ alkyl optionally substituted with hydroxyl, oxo, or dialkyl amino;
R¹ is optionally substituted pyrazol-1-yl, phenyl optionally substituted with optionally substituted C₂-C₉ heteroaryl or optionally substituted C₆-C₁₀ aryl, or —N(R^{1A})N═C(R^{1B})₂; and
R³ is

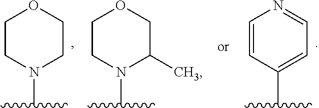

In some embodiments, R³ is

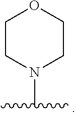 [SMILES: *N1CCOCC1]

In some embodiments, R³ is

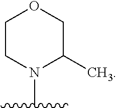 [SMILES: *N1CCOCC1C]

In some embodiments, R³ is

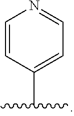 [SMILES: *c1ccncc1]

In some embodiments, R² is hydrogen. In some embodiments, R² is optionally substituted C₂-C₉ heteroaryl. In some embodiments, R² is pyridin-4-yl. In some embodiments, R² is optionally substituted C₂-C₉ heterocyclyl. In some embodiments, R² is

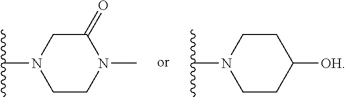

In some embodiments, R² is C₁-C₃ alkyl optionally substituted with hydroxyl, oxo, or dialkyl amino. In some embodiments, R² is

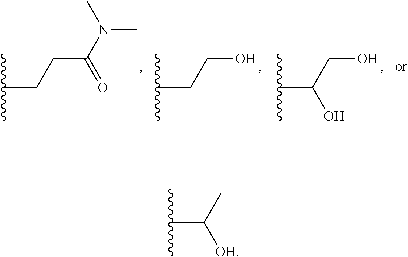

In some embodiments, R¹ is optionally substituted pyrazol-1-yl. In some embodiments, R¹ is

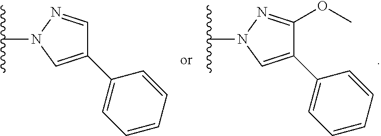 [SMILES: *n1cc(-c2ccccc2)cn1 ; *n1cc(-c2ccccc2)c(OC)n1]

In some embodiments, R¹ is phenyl optionally substituted with optionally substituted C₂-C₉ heteroaryl or optionally substituted C₆-C₁₀ aryl. In some embodiments, R¹ is

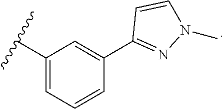 [SMILES: *c1cccc(-c2ccn(C)n2)c1]

In some embodiments, R¹ is —N(R^{1A})N═C(R^{1B})₂. In some embodiments, R¹ is

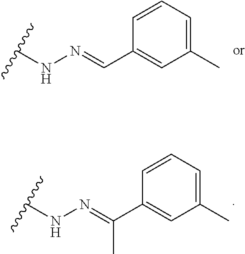 [SMILES: *N/N=C/c1cccc(C)c1 ; *N/N=C(\C)c1cccc(C)c1]

In some embodiments, the compound has the structure:

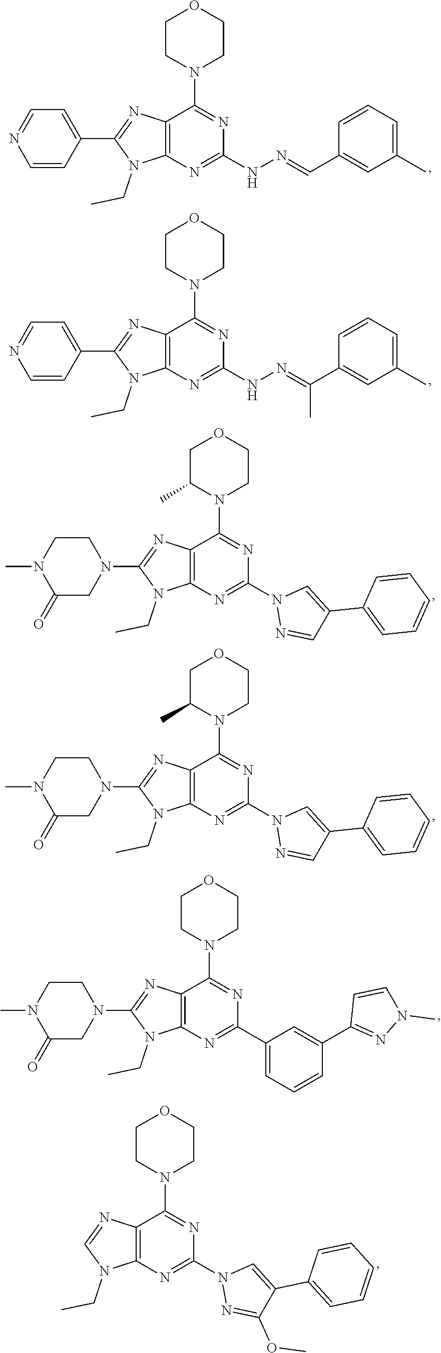

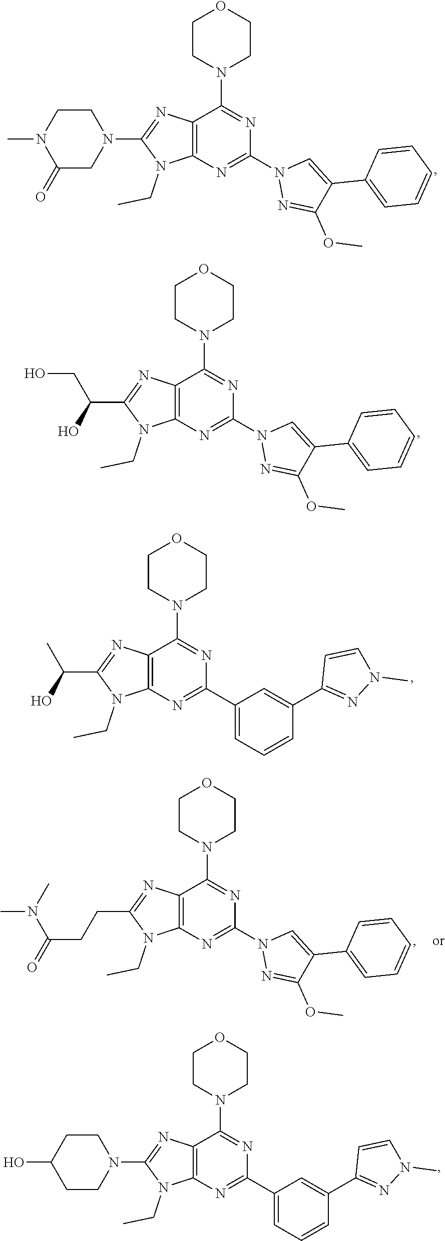

or a pharmaceutically acceptable salt thereof.

In some embodiments, the compound has the structure:

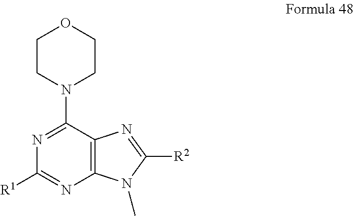

or a pharmaceutically acceptable salt thereof,
where R² is optionally substituted C₂-C₉ heteroaryl; and
R¹ is —N(R^{1A})N═C(R^{1B})₂.

In some embodiments, R² is optionally substituted pyridine-4-yl. In some embodiments, R² is pyridine-4-yl. In some embodiments, R¹ is

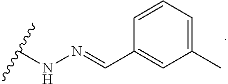 [SMILES: *N/N=C/c1cccc(C)c1]

In some embodiments, the compound has the structure:

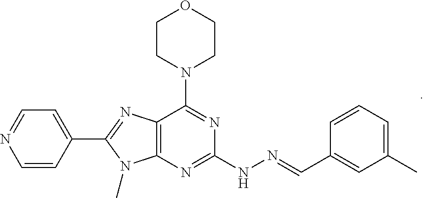 [SMILES: Cc1cccc(/C=N/Nc2nc(N3CCOCC3)c3nc(-c4ccncc4)n(C)c3n2)c1]

In some embodiments, the compound has the structure of any one of compounds 1, 2, 14-22, 31, 44-46, 48-52, 56, 57, 60, 76-82, 93-96, 98, 108, 109, 116, 126, 133-139, 147-149, 157-163, 165-169, 171-180, 186, 195-197, 262, 286, 287, 291, 292, 294-299, 325, 329, 464, 465, and 467-473 in Table 1 or a pharmaceutically acceptable salt thereof.

In some embodiments, the compound has the structure of any one of compounds 3-13, 24-30, 32-43, 47, 53-55, 58, 59, 61-75, 83-92, 97, 99-107, 110-115, 117-125, 127-132, 140-146, 450-156, 181-185, 187-194, 198-261, 263-285, 288-290, 293, 300-324, 326-328, 330-390, 392-463, and 466 in Table 1 or a pharmaceutically acceptable salt thereof.

In an aspect, the invention provides a compound having the structure having the structure

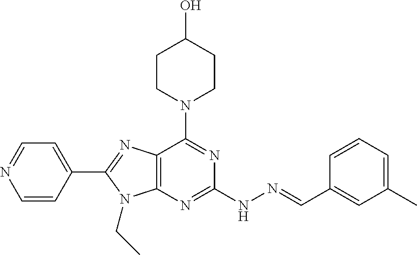 [SMILES: CCn1c(-c2ccncc2)nc2c(N3CCC(O)CC3)nc(N/N=C/c3cccc(C)c3)nc21]

or a pharmaceutically acceptable salt thereof.

In an aspect, the invention features a pharmaceutical composition including any of the foregoing compounds and a pharmaceutically acceptable excipient.

In an aspect, the invention features a method of treating a neurological disorder (e.g., frontotemporal dementia (FTLD-TDP), chronic traumatic encephalopathy, ALS, Alzheimer's disease, limbic-predominant age-related TDP-43 encephalopathy (LATE), or frontotemporal lobar degeneration) in a subject in need thereof. This method includes administering an effective amount of any of the foregoing compounds or pharmaceutical compositions.

In an aspect, the invention features a method of inhibiting toxicity in a cell (e.g., mammalian neural cell) related to a protein (e.g., TDP-43 or C9orf72). This method includes administering an effective amount of any of the foregoing compounds or pharmaceutical compositions.

In an aspect, the invention features a method of treating a TDP-43-associated disorder or C9orf72-associated disorder (e.g., FTLD-TDP, chronic traumatic encephalopathy, ALS, Alzheimer's disease, LATE, or frontotemporal lobar degeneration) in a subject in need thereof. This method includes administering to the subject an effective amount of a compounds described herein or a pharmaceutical composition containing one or more compounds described herein. In some embodiments, the method includes administering to the subject in need thereof an effective amount of the compound of formula 49

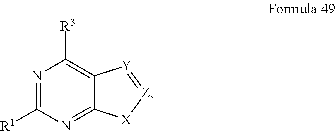

or a pharmaceutically acceptable salt thereof,
where
X is NR^{A}, S, or O;
Y is CR^{A} or N;
Z is CR² or N;
R¹ is hydrogen, optionally substituted C₁-C₆ alkyl, optionally substituted C₆-C₁₀ aryl, optionally substituted C₁-C₉ heterocyclyl, optionally substituted amino, optionally substituted C₃-C₈ cycloalkyl, optionally substituted C₂-C₉ heterocyclyl C₁-C₆ alkyl, optionally substituted C₂-C₉ heteroaryl C₁-C₆ alkyl, optionally substituted C₂-C₉ heteroaryl; —NHNHR^{1A}; —N(R^{1A})N═C(R^{1B})₂; —C(R^{1A})═N—N(R^{1B})₂; —C(R^{1A})═NOR^{1A}; or -Q¹-N(R^{1C})₂;
Q¹ is a bond, CH₂, or CO;
each R^{1A} is independently H, optionally substituted C₁-C₆ alkyl, optionally substituted C₆-C₁₀ aryl, or optionally substituted C₆-C₁₀ aryl C₁-C₆ alkyl;
one R^{1B} is independently H, optionally substituted C₁-C₆ alkyl, optionally substituted C₆-C₁₀ aryl, or optionally substituted C₂-C₉ heteroaryl; and the remaining R^{1B} is optionally substituted 01-C₆ alkyl, optionally substituted C₆-C₁₀ aryl, or optionally substituted C₂-C₉ heteroaryl;
each R^{1C} is independently H, optionally substituted C₁-C₆ alkyl, optionally substituted C₆-C₁₀ aryl, optionally substituted C₃-C₈ cycloalkyl, or optionally substituted C₂-C₉ heteroaryl; or both R^{1C}, together with the nitrogen atom to which they are attached, combine to form C₂-C₉ heterocyclyl or C₂-C₉ heteroaryl;
R² is optionally substituted C₁-C₆ alkyl, optionally substituted C₆-C₁₀ aryl, optionally substituted C₂-C₉ heterocyclyl, optionally substituted C₂-C₉ heteroaryloxy, optionally substituted C₃-C₈ cycloalkyl, optionally substituted C₃-C₈ cycloalkenyl, or optionally substituted C₂-C₉ heteroaryl, -Q-N(R^{1c})₂; —S(O)ᵣ—R^{1A}; or —P(O)(R^{1A})₂; and each R^{A} is independently H, optionally substituted C₁-C₆ alkyl, optionally substituted C₆-C₁₀ aryl, optionally substituted C₂-C₉ heteroaryl, optionally substituted C₂-C₉ heteroaryl C₁-C₆ alkyl, optionally substituted C₃-C₈ cycloalkyl; or R² and R^{A}, together with the atoms to which they are attached, combine to form an optionally substituted C₃-C₄ heterocyclic ring, and the remaining R^{A}, if present, is H, optionally substituted C₁-C₆ alkyl, optionally substituted C₆-C₁₀ aryl, optionally substituted C₂-C₉ heteroaryl, optionally substituted C₂-C₉ heteroaryl C₁-C₆ alkyl, optionally substituted C₃-C₈ cycloalkyl;
r is 0, 1, or 2;
R³ is

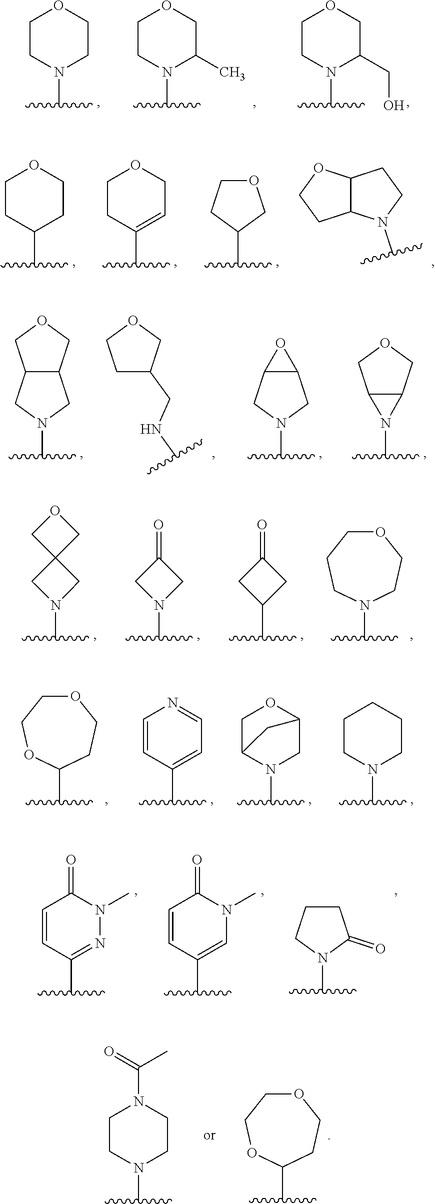

In some embodiments, the compound is of formula 49a:

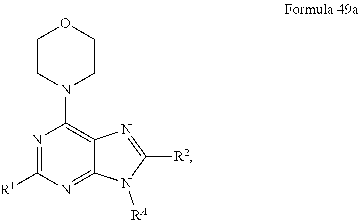

or a pharmaceutically acceptable salt thereof.

In some embodiments, R^{A} is optionally substituted C₁-C₆ alkyl. In some embodiments, R^{A} is H.

In some embodiments, the compound is of formula 49b:

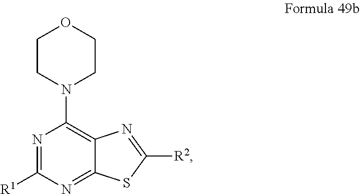

or a pharmaceutically acceptable salt thereof.

In some embodiments, the compound is of formula 49c:

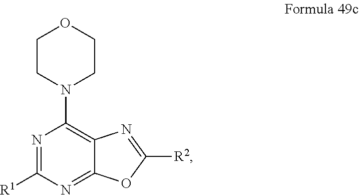

or a pharmaceutically acceptable salt thereof.

In some embodiments, the compound is of formula 49d:

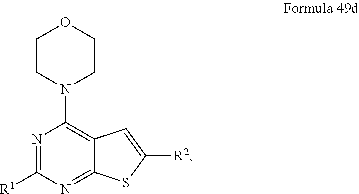

or a pharmaceutically acceptable salt thereof.

In some embodiments, R¹ is optionally substituted C₂-C₉ heteroaryl including a 5-membered ring having a nitrogen atom at position 2 relative to the bond to the core.

In some embodiments, R¹ is optionally substituted pyrazol-1-yl, optionally substituted pyrazol-3-yl, optionally substituted 1,2,3-triazol-1-yl, optionally substituted 1,2,3-traizol-2-yl, optionally substituted benzotriazole-1-yl, optionally substituted 1,2,4 triazol-3-yl, optionally substituted 1,2,4-oxadizol-3-yl, optionally substituted, 1,2,4-oxadizol-2-yl. In some embodiments, R¹ is pyrazol-1-yl substituted at position 3 or position 4. In some embodiments, the pyrazol-1-yl is optionally substituted with optionally substituted C₆-C₁₀ aryl, optionally substituted C₁₋₉ heterocyclyl, optionally substituted C₂-C₉ heteroaryl, optionally substituted C₃₋₈ cycloalkyl, or halo (e.g., fluoro, chloro, bromo). In some embodiments, R¹ is optionally substituted pyrazol-3-yl. In some embodiments, R¹ is pyrazol-3-yl substituted at position 1.

In some embodiments, the optionally substituted pyrazol-1-yl is

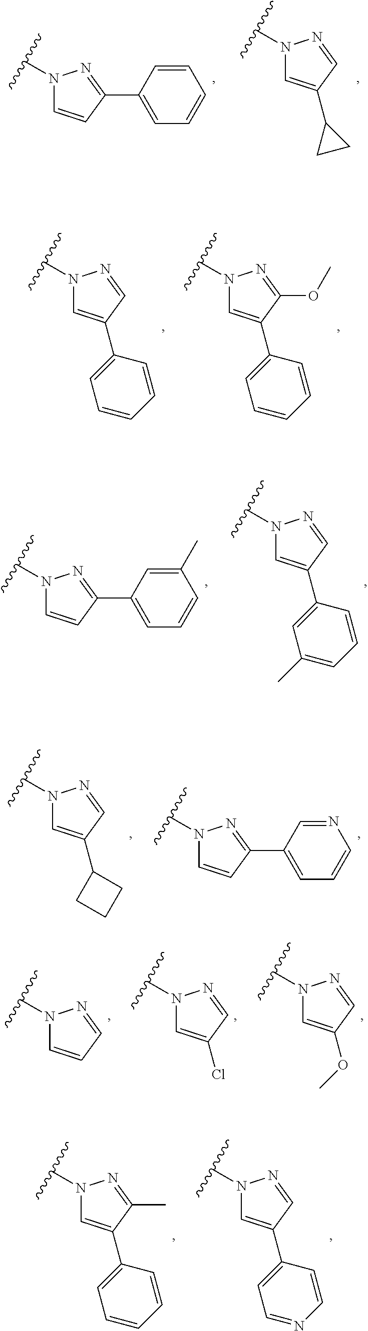

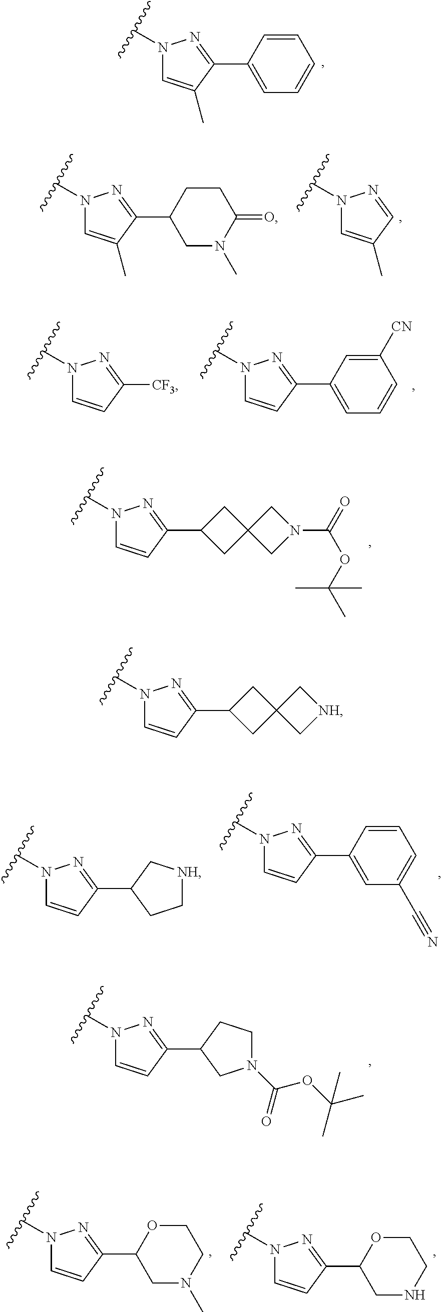

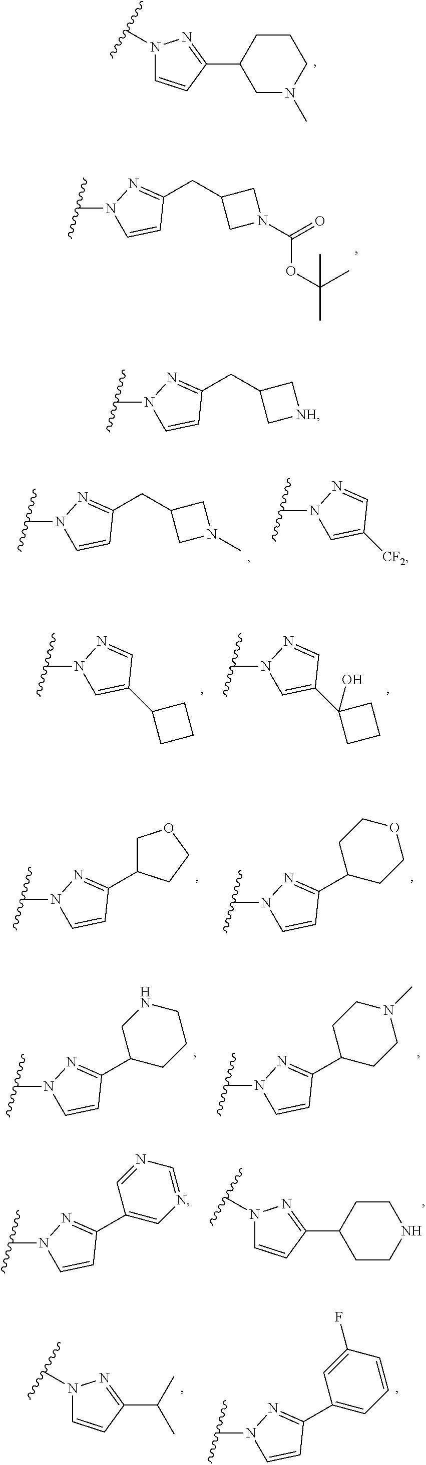

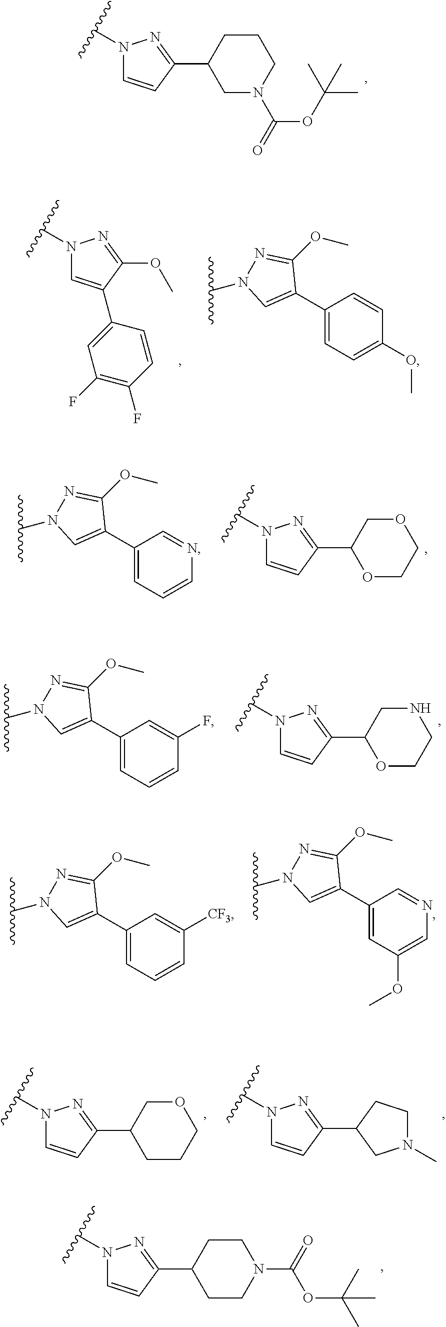

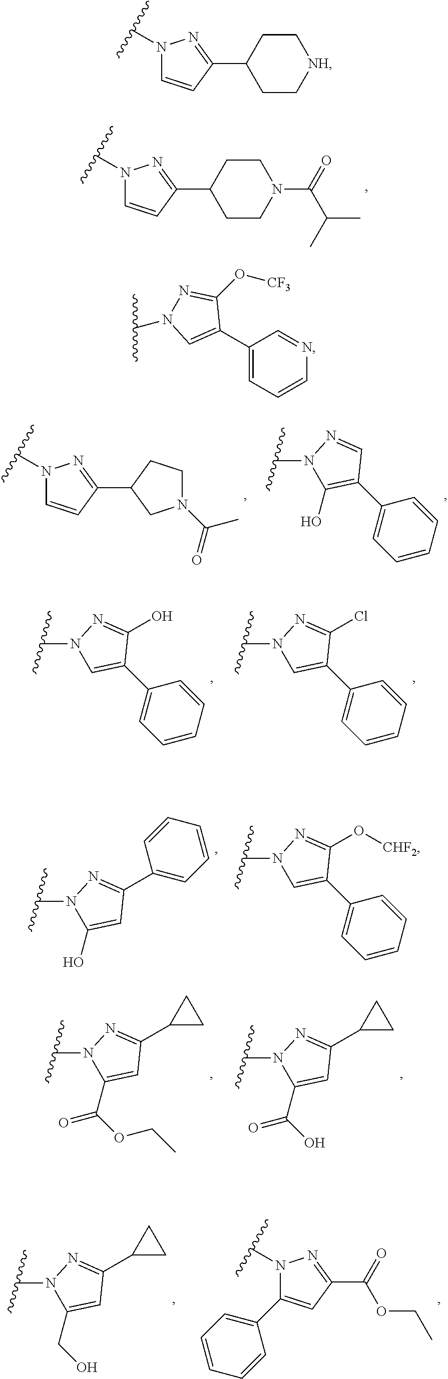

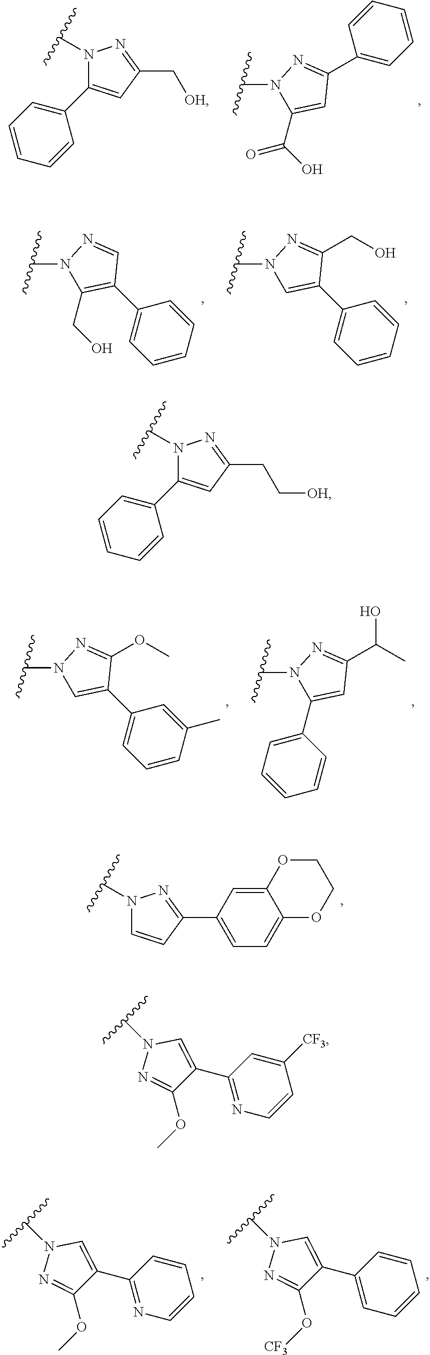

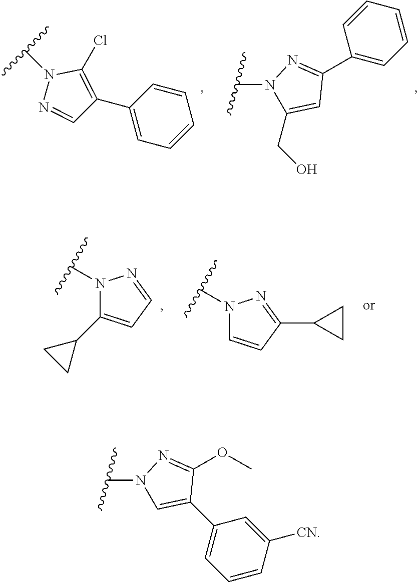 [SMILES: *n1ncc(-c2ccccc2)c1Cl ; *n1nc(-c2ccccc2)cc1CO ; *n1nccc1C1CC1 ; *n1ccc(C2CC2)n1 ; *n1cc(-c2cccc(C#N)c2)c(OC)n1]

In some embodiments, the pyrazol-3-yl substituted with optionally substituted C₆-C₁₀ aryl, optionally substituted C₁₋₉ heterocyclyl, optionally substituted C₂-C₉ heteroaryl, or optionally substituted C₃₋₈ cycloalkyl. In some embodiments, the pyrazol-3-yl is

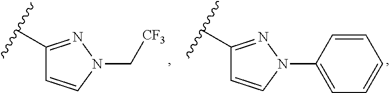 [SMILES: *c1ccn(CC(F)(F)F)n1 ; *c1ccn(-c2ccccc2)n1]

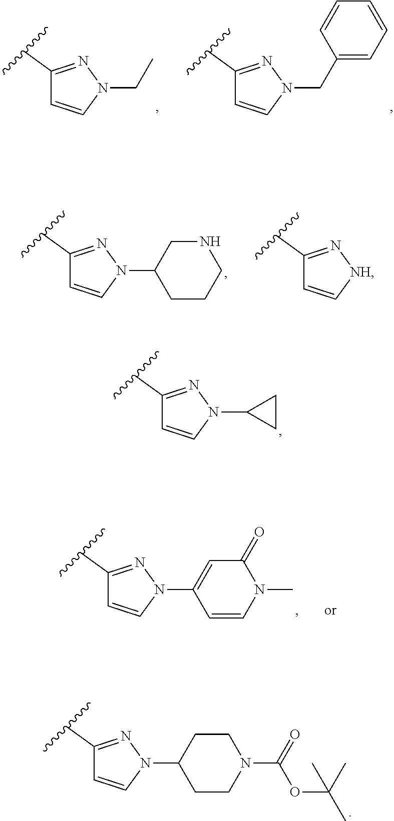

In some embodiments, R¹ is optionally substituted pyrimidin-6-yl or optionally substituted pyrimidin-4-yl. In some embodiments, R¹ is

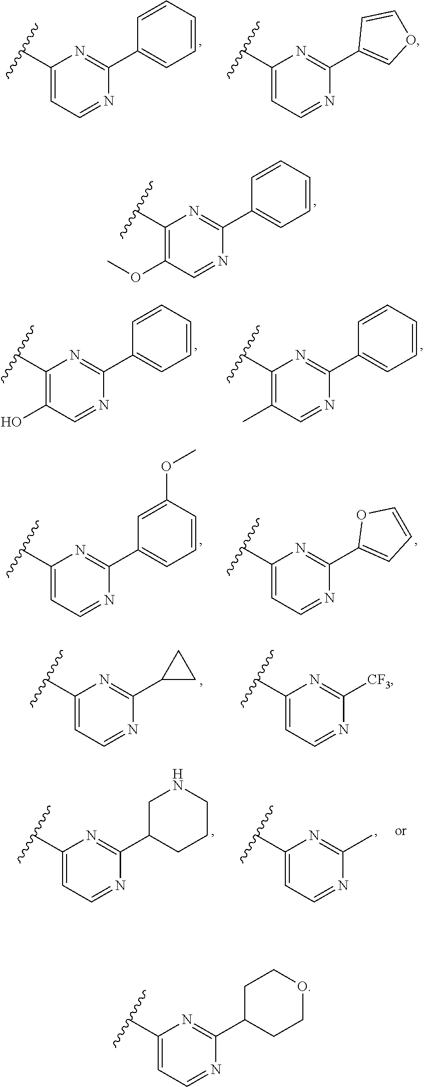

In some embodiments, R² is optionally substituted C₂-C₉ heteroaryl. In some embodiments, R² is optionally substituted pyridyl. In some embodiments, R² is optionally substituted tetrahydropyranyl, optionally substituted dihydropyranyl, optionally substituted piperidinyl, or optionally substituted azetidinyl. In some embodiments, R² is optionally substituted tetrahydropyran-4-yl, optionally substituted 5,6-dihydro-2H-pyran-4-yl, optionally substituted piperidin-4-yl, or optionally substituted piperidin-3-yl. In some embodiments, R¹ is phenyl substituted with methoxy, optionally substituted C₁-C₆ alkyl, hydroxyl, optionally substituted C₂-C₉ heteroaryl, optionally substituted C₆-C₁₀ aryl, optionally substituted C₂-C₉ heterocyclyl, or C₃-C₈ cycloalkoxy. In some embodiments, phenyl is substituted with C₂-C₉ heteroaryl. In some embodiments, R¹ is

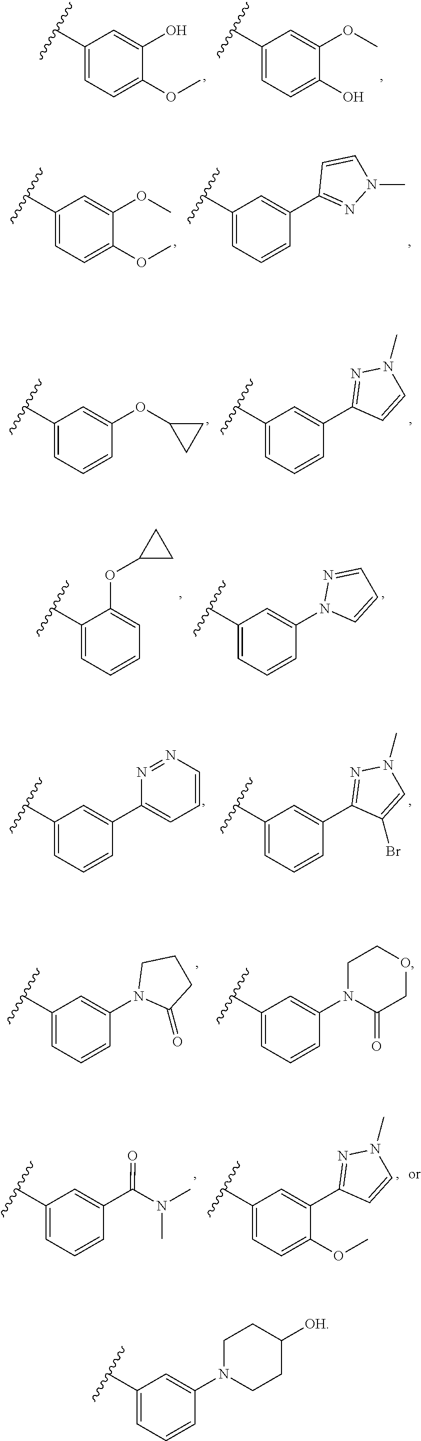

In an aspect, the invention features a method of inhibiting PIKfyve. This method includes contacting a cell with an effective amount of any of the foregoing compounds or pharmaceutical compositions.

In another aspect, the invention features a method of treating a neurological disorder in a patient, such as a human patient, identified as likely to benefit from treatment with a compound of the invention on the basis of TDP-43 toxicity. In this aspect, the method may include (i) determining that the patient exhibits, or is prone to develop, TDP-43 toxicity, and (ii) providing to the patient a therapeutically effective amount of a compound of the invention. In some embodiments, the patient has previously been determined to exhibit, or to be prone to developing, TDP-43 toxicity, and the method includes providing to the patient a therapeutically effective amount of a compound of the invention. The susceptibility of the patient to developing TDP-43 aggregation may be determined, e.g., by determining whether the patient expresses a mutant isoform of TDP-43 containing a mutation that is associated with TDP-43 aggregation and toxicity, such as a mutation selected from Q331 K, M337V, Q343R, N345K, R361S, and N390D. This may be performed, for example, by determining the amino acid sequence of a TDP-43 isoform isolated from a sample obtained from the patient or by determining the nucleic acid sequence of a TDP-43 gene isolated from a sample obtained from the patient. In some embodiments, the method includes the step of obtaining the sample from the patient.

In an additional aspect, the invention features a method of treating a neurological disorder in a patient, such as a human patient, identified as likely to benefit from treatment with a compound of the invention on the basis of TDP-43 expression. In this aspect, the method includes (i) determining that the patient expresses a mutant form of TDP-43 having a mutation associated with TDP-43 aggregation (e.g., a mutation selected from Q331K, M337V, Q343R, N345K, R361S, and N390D), and (ii) providing to the patient a therapeutically effective amount of a compound of the invention. In some embodiments, the patient has previously been determined to express a mutant form of TDP-43 having a mutation associated with TDP-43 aggregation, such as a Q331 K, M337V, Q343R, N345K, R361 S, or N390D mutation, and the method includes providing to the patient a therapeutically effective amount of a compound of the invention.

In another aspect, the invention features a method of determining whether a patient (e.g., a human patient) having a neurological disorder is likely to benefit from treatment with a compound of the invention by (i) determining whether the patient exhibits, or is prone to develop, TDP-43 aggregation and (ii) identifying the patient as likely to benefit from treatment with a compound of the invention if the patient exhibits, or is prone to develop, TDP-43 aggregation. In some embodiments, the method further includes the step of (iii) informing the patient whether he or she is likely to benefit from treatment with a compound of the invention. The susceptibility of the patient to developing TDP-43 aggregation may be determined, e.g., by determining whether the patient expresses a mutant isoform of TDP-43 containing a mutation that is associated with TDP-43 aggregation and toxicity, such as a mutation selected from Q331 K, M337V, Q343R, N345K, R361S, and N390D. This may be performed, for example, by determining the amino acid sequence of a TDP-43 isoform isolated from a sample obtained from the patient or by determining the nucleic acid sequence of a TDP-43 gene isolated from a sample obtained from the patient. In some embodiments, the method includes the step of obtaining the sample from the patient.

In another aspect, the invention features a method of determining whether a patient (e.g., a human patient) having a neurological disorder is likely to benefit from treatment with a compound of the invention by (i) determining whether the patient expresses a TDP-43 mutant having a mutation associated with TDP-43 aggregation (e.g., a mutation selected from Q331K, M337V, Q343R, N345K, R361S, and N390D) and (ii) identifying the patient as likely to benefit from treatment with a compound of the invention if the patient expresses a TDP-43 mutant. In some embodiments, the method further includes the step of (iii) informing the patient whether he or she is likely to benefit from treatment with a compound of the invention. The TDP-43 isoform expressed by the patient may be assessed, for example, by isolated TDP-43 protein from a sample obtained from the patient and sequencing the protein using molecular biology techniques described herein or known in the art. In some embodiments, the TDP-43 isoform expressed by the patient is determined by analyzing the patient's genotype at the TDP-43 locus, for example, by sequencing the TDP-43 gene in a sample obtained from the patient. In some embodiments, the method includes the step of obtaining the sample from the patient.

In some embodiments of any of the above aspects, the compound of the invention is provided to the patient by administration of the compound of the invention to the patient. In some embodiments, the compound of the invention is provided to the patient by administration of a prodrug that is converted in vivo to the compound of the invention.

In some embodiments of any of the above aspects, the neurological disorder is a neuromuscular disorder, such as a neuromuscular disorder selected from amyotrophic lateral sclerosis, congenital myasthenic syndrome, congenital myopathy, cramp fasciculation syndrome, Duchenne muscular dystrophy, glycogen storage disease type II, hereditary spastic paraplegia, inclusion body myositis, Isaac's Syndrome, Kearns-Sayre syndrome, Lambert-Eaton myasthenic syndrome, mitochondrial myopathy, muscular dystrophy, myasthenia gravis, myotonic dystrophy, peripheral neuropathy, spinal and bulbar muscular atrophy, spinal muscular atrophy, Stiff person syndrome, Troyer syndrome, and Guillain-Barre syndrome. In some embodiments, the neurological disorder is amyotrophic lateral sclerosis.

In some embodiments of any of the above aspects, the neurological disorder is selected from frontotemporal degeneration (also referred to as frontotemporal lobar degeneration and frontotemporal dementia), Alzheimer's disease, Parkinson's disease, dementia with Lewy Bodies, corticobasal degeneration, progressive supranuclear palsy, dementia parkinsonism ALS complex of Guam, Huntington's disease, Inclusion body myopathy with early-onset Paget disease and frontotemporal dementia (IBMPFD), sporadic inclusion body myositis, myofibrillar myopathy, dementia pugilistica, chronic traumatic encephalopathy, Alexander disease, and hereditary inclusion body myopathy.

In some embodiments, the neurological disorder is amyotrophic lateral sclerosis, and following administration of the compound of the invention to the patient, the patient exhibits one or more, or all, of the following responses:
(i) an improvement in condition as assessed using the amyotrophic lateral sclerosis functional rating scale (ALSFRS) or the revised ALSFRS (ALSFRS-R), such as an improvement in the patient's ALSFRS or ALSFRS-R score within one or more days, weeks, or months following administration of the compound of the invention (e.g., an improvement in the patient's ALSFRS or ALSFRS-R score within from about 1 day to about 48 weeks (e.g., within from about 2 days to about 36 weeks, from about 4 weeks to about 24 weeks, from about 8 weeks to about 20 weeks, or from about 12 weeks to about 16 weeks), or more, following the initial administration of the compound of the invention to the patient, such as within 1 day, 2 days, 3 days, 4 days, 5 days, 6 days, 7 days, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, or more, following the initial administration of the compound of the invention to the patient);
(ii) an increase in slow vital capacity, such as an increase in the patient's slow vital capacity within one or more days, weeks, or months following administration of the compound of the invention (e.g., an increase in the patient's slow vital capacity within from about 1 day to about 48 weeks (e.g., within from about 2 days to about 36 weeks, from about 4 weeks to about 24 weeks, from about 8 weeks to about 20 weeks, or from about 12 weeks to about 16 weeks), or more, following the initial administration of the compound of the invention to the patient, such as within 1 day, 2 days, 3 days, 4 days, 5 days, 6 days, 7 days, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, or more, following the initial administration of the compound of the invention to the patient);
(iii) a reduction in decremental responses exhibited by the patient upon repetitive nerve stimulation, such as a reduction that is observed within one or more days, weeks, or months following administration of the compound of the invention (e.g., a reduction that is observed within from about 1 day to about 48 weeks (e.g., within from about 2 days to about 36 weeks, from about 4 weeks to about 24 weeks, from about 8 weeks to about 20 weeks, or from about 12 weeks to about 16 weeks), or more, following the initial administration of the compound of the invention to the patient, such as within 1 day, 2 days, 3 days, 4 days, 5 days, 6 days, 7 days, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, or more, following the initial administration of the compound of the invention to the patient);
(iv) an improvement in muscle strength, as assessed, for example, by way of the Medical Research Council muscle testing scale (as described, e.g., in Jagtap et al., Ann. Indian. Acad. Neurol. 17:336-339 (2014), the disclosure of which is incorporated herein by reference as it pertains to measuring patient response to neurological disease treatment), such as an improvement that is observed within one or more days, weeks, or months following administration of the compound of the invention (e.g., an improvement that is observed within from about 1 day to about 48 weeks (e.g., within from about 2 days to about 36 weeks, from about 4 weeks to about 24 weeks, from about 8 weeks to about 20 weeks, or from about 12 weeks to about 16 weeks), or more, following the initial administration of the compound of the invention to the patient, such as within 1 day, 2 days, 3 days, 4 days, 5 days, 6 days, 7 days, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, or more, following the initial administration of the compound of the invention to the patient);
(v) an improvement in quality of life, as assessed, for example, using the amyotrophic lateral sclerosis-specific quality of life (ALS-specific QOL) questionnaire, such as an improvement in the patient's quality of life that is observed within one or more days, weeks, or months following administration of the compound of the invention (e.g., an improvement in the subject's quality of life that is observed within from about 1 day to about 48 weeks (e.g., within from about 2 days to about 36 weeks, from about 4 weeks to about 24 weeks, from about 8 weeks to about 20 weeks, or from about 12 weeks to about 16 weeks), or more, following the initial administration of the compound of the invention to the patient, such as within 1 day, 2 days, 3 days, 4 days, 5 days, 6 days, 7 days, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, or more, following the initial administration of the compound of the invention to the patient);
(vi) a decrease in the frequency and/or severity of muscle cramps, such as a decrease in cramp frequency and/or severity within one or more days, weeks, or months following administration of the compound of the invention (e.g., a decrease in cramp frequency and/or severity within from about 1 day to about 48 weeks (e.g., within from about 2 days to about 36 weeks, from about 4 weeks to about 24 weeks, from about 8 weeks to about 20 weeks, or from about 12 weeks to about 16 weeks), or more, following the initial administration of the compound of the invention to the patient, such as within 1 day, 2 days, 3 days, 4 days, 5 days, 6 days, 7 days, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, or more, following the initial administration of the compound of the invention to the patient); and/or
(vii) a decrease in TDP-43 aggregation, such as a decrease in TDP-43 aggregation within one or more days, weeks, or months following administration of the compound of the invention (e.g., a decrease in TDP-43 aggregation within from about 1 day to about 48 weeks (e.g., within from about 2 days to about 36 weeks, from about 4 weeks to about 24 weeks, from about 8 weeks to about 20 weeks, or from about 12 weeks to about 16 weeks), or more, following the initial administration of the compound of the invention to the patient, such as within 1 day, 2 days, 3 days, 4 days, 5 days, 6 days, 7 days, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, or more, following the initial administration of the compound of the invention to the patient.

### Chemical Terms

It is to be understood that the terminology employed herein is for the purpose of describing particular embodiments and is not intended to be limiting.

Those skilled in the art will appreciate that certain compounds described herein can exist in one or more different isomeric (e.g., stereoisomers, geometric isomers, tautomers) and/or isotopic (e.g., in which one or more atoms has been substituted with a different isotope of the atom, such as hydrogen substituted for deuterium) forms. Unless otherwise indicated or clear from context, a depicted structure can be understood to represent any such isomeric or isotopic form, individually or in combination.

In some embodiments, one or more compounds depicted herein may exist in different tautomeric forms. As will be clear from context, unless explicitly excluded, references to such compounds encompass all such tautomeric forms. In some embodiments, tautomeric forms result from the swapping of a single bond with an adjacent double bond and the concomitant migration of a proton. In certain embodiments, a tautomeric form may be a prototropic tautomer, which is an isomeric protonation states having the same empirical formula and total charge as a reference form. Examples of moieties with prototropic tautomeric forms are ketone—enol pairs, amide—imidic acid pairs, lactam—lactim pairs, amide—imidic acid pairs, enamine—imine pairs, and annular forms where a proton can occupy two or more positions of a heterocyclic system, such as, 1H- and 3H-imidazole, 1H-, 2H- and 4H-1,2,4-triazole, 1H- and 2H-isoindole, and 1H- and 2H-pyrazole. In some embodiments, tautomeric forms can be in equilibrium or sterically locked into one form by appropriate substitution. In certain embodiments, tautomeric forms result from acetal interconversion, e.g., the interconversion illustrated in the scheme below:

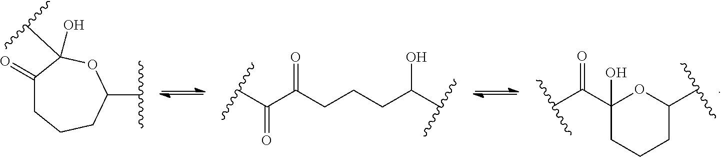 [SMILES: *C1CCCC(=O)C(*)(O)O1 ; *C(=O)C(=O)CCCC(*)O ; *C(=O)C1(O)CCCC(*)O1]

Those skilled in the art will appreciate that, in some embodiments, isotopes of compounds described herein may be prepared and/or utilized in accordance with the present invention. “Isotopes” refers to atoms having the same atomic number but different mass numbers resulting from a different number of neutrons in the nuclei. For example, isotopes of hydrogen include tritium and deuterium. In some embodiments, an isotopic substitution (e.g., substitution of hydrogen with deuterium) may alter the physiciochemical properties of the molecules, such as metabolism and/or the rate of racemization of a chiral center.

As is known in the art, many chemical entities (in particular many organic molecules and/or many small molecules) can adopt a variety of different solid forms such as, for example, amorphous forms and/or crystalline forms (e.g., polymorphs, hydrates, solvates, etc). In some embodiments, such entities may be utilized in any form, including in any solid form. In some embodiments, such entities are utilized in a particular form, e.g., in a particular solid form.

In some embodiments, compounds described and/or depicted herein may be provided and/or utilized in salt form.

In certain embodiments, compounds described and/or depicted herein may be provided and/or utilized in hydrate or solvate form.

At various places in the present specification, substituents of compounds of the present disclosure are disclosed in groups or in ranges. It is specifically intended that the present disclosure include each and every individual subcombination of the members of such groups and ranges. For example, the term “C₁-C₆ alkyl” is specifically intended to individually disclose methyl, ethyl, C₃ alkyl, C₄ alkyl, C₅ alkyl, and C₈ alkyl. Furthermore, where a compound includes a plurality of positions at which substitutes are disclosed in groups or in ranges, unless otherwise indicated, the present disclosure is intended to cover individual compounds and groups of compounds (e.g., genera and subgenera) containing each and every individual subcombination of members at each position.

Herein a phrase of the form “optionally substituted X” (e.g., optionally substituted alkyl) is intended to be equivalent to “X, where X is optionally substituted” (e.g., “alkyl, where said alkyl is optionally substituted”). It is not intended to mean that the feature “X” (e.g., alkyl) per se is optional.

The term “acyl,” as used herein, represents a hydrogen or an alkyl group, as defined herein that is attached to a parent molecular group through a carbonyl group, as defined herein, and is exemplified by formyl (i.e., a carboxyaldehyde group), acetyl, trifluoroacetyl, propionyl, and butanoyl. Exemplary unsubstituted acyl groups include from 1 to 6, from 1 to 11, or from 1 to 21 carbons.

The term “alkyl,” as used herein, refers to a branched or straight-chain monovalent saturated aliphatic hydrocarbon radical of 1 to 20 carbon atoms (e.g., 1 to 16 carbon atoms, 1 to 10 carbon atoms, or 1 to 6 carbon atoms). An alkylene is a divalent alkyl group.

The term “alkenyl,” as used herein, alone or in combination with other groups, refers to a straight-chain or branched hydrocarbon residue having a carbon-carbon double bond and having 2 to 20 carbon atoms (e.g., 2 to 16 carbon atoms, 2 to 10 carbon atoms, 2 to 6, or 2 carbon atoms).

The term “alkynyl,” as used herein, alone or in combination with other groups, refers to a straight-chain or branched hydrocarbon residue having a carbon-carbon triple bond and having 2 to 20 carbon atoms (e.g., 2 to 16 carbon atoms, 2 to 10 carbon atoms, 2 to 6, or 2 carbon atoms).

The term “amino,” as used herein, represents —N(R^{N1})₂, where each R^{N1} is, independently, H, OH, NO₂, N(R^{N2})₂, SO₂OR^{N2}, SO₂R^{N2}, SOR^{N2}, an N-protecting group, alkyl, alkoxy, aryl, arylalkyl, cycloalkyl, acyl (e.g., acetyl, trifluoroacetyl, or others described herein), where each of these recited R^{N1} groups can be optionally substituted; or two R^{N1} combine to form an alkylene or heteroalkylene, and where each R^{N2} is, independently, H, alkyl, or aryl. The amino groups of the invention can be an unsubstituted amino (i.e., —NH₂) or a substituted amino (i.e., —N(R^{N1})₂). An amino group, having one R¹ are H and the other R^{N1} as a non-H group, may be referred to as a monosubstituted amino. For example, when one R^{N1} is H, and the other R^{N1} is optionally substituted alkyl, the resulting amino group is an optionally substitute monoalkylamino. When both R^{N1} groups are independently optionally substituted alkyls, the resulting amino group is an optionally substituted dialkylamino.

The term “aryl,” as used herein, refers to an aromatic mono- or polycarbocyclic radical of 6 to 12 carbon atoms having at least one aromatic ring. Examples of such groups include, but are not limited to, phenyl, naphthyl, 1,2,3,4-tetrahydronaphthyl, 1,2-dihydronaphthyl, indanyl, and 1H-indenyl.

The term “arylalkyl,” as used herein, represents an alkyl group substituted with an aryl group. Exemplary unsubstituted arylalkyl groups are from 7 to 30 carbons (e.g., from 7 to 16 or from 7 to 20 carbons, such as C₆-C₁₀ aryl C₁-C₆ alkyl, C₆-C₁₀ aryl C₁-C₁₀ alkyl, or C₆-C₁₀ aryl C₁-C₂₀ alkyl), such as, benzyl and phenethyl. In some embodiments, the akyl and the aryl each can be further substituted with 1, 2, 3, or 4 substituent groups as defined herein for the respective groups.

The term “aryloxy”, as used herein, refers to an oxygen atom substituted with an aryl group, as defined herein, e.g., —O-phenyl, or —O-naphthyl.

The term “azido,” as used herein, represents a —N₃ group.

The term “cyano,” as used herein, represents a CN group.

The term “carbocyclyl,” as used herein, refer to a non-aromatic C₃-C₁₂ monocyclic, bicyclic, or tricyclic structure in which the rings are formed by carbon atoms. Carbocyclyl structures include cycloalkyl groups and unsaturated carbocyclyl radicals.

The term “cycloalkenyl,” as used herein, refers to a non-aromatic, monovalent mono- or polycarbocyclic radical of three to ten, preferably three to six carbon atoms, and one or two endocyclic carbon-carbon double bonds. This term is further exemplified by radicals such as cycloheptenyl, cyclohexenyl, and cyclopentenyl. A polycyclic cycloalkenyl may be fused, bridged, or spiro cycloalkenyl.

The term “cycloalkyl,” as used herein, refers to a saturated, non-aromatic, monovalent mono- or polycarbocyclic radical of three to ten, preferably three to six carbon atoms. This term is further exemplified by radicals such as cyclopropyl, cyclobutyl, cyclopentyl, cyclohexyl, cycloheptyl, norbornyl, and adamantyl. A polycyclic cycloalkyl may be fused, bridged, or spiro cycloalkyl.

The term “cycloalkoxy”, as used herein, refers to an oxygen atom substituted with a cycloalkyl group, as defined herein, e.g., —O-cyclopropyl, —O-cyclobutyl, —O-cyclopentyl, or —O-cyclohexyl.

The term “halo,” as used herein, means a fluorine (fluoro), chlorine (chloro), bromine (bromo), or iodine (iodo) radical.

The term “heteroalkyl,” as used herein, refers to an alkyl group, as defined herein, in which one or more of the constituent carbon atoms have been replaced by nitrogen, oxygen, or sulfur. In some embodiments, the heteroalkyl group can be further substituted with 1, 2, 3, or 4 substituent groups as described herein for alkyl groups. Examples of heteroalkyl groups are an “alkoxy” which, as used herein, refers alkyl-O— (e.g., methoxy and ethoxy). A heteroalkylene is a divalent heteroalkyl group.

The term “heteroalkenyl,” as used herein, refers to an alkenyl group, as defined herein, in which one or more of the constituent carbon atoms have been replaced by nitrogen, oxygen, or sulfur. In some embodiments, the heteroalkenyl group can be further substituted with 1, 2, 3, or 4 substituent groups as described herein for alkenyl groups. Examples of heteroalkenyl groups are an “alkenoxy” which, as used herein, refers alkenyl-O—. A heteroalkenylene is a divalent heteroalkenyl group.

The term “heteroalkynyl,” as used herein, refers to an alkynyl group, as defined herein, in which one or more of the constituent carbon atoms have been replaced by nitrogen, oxygen, or sulfur. In some embodiments, the heteroalkynyl group can be further substituted with 1, 2, 3, or 4 substituent groups as described herein for alkynyl groups. Examples of heteroalkynyl groups are an “alkynoxy” which, as used herein, refers alkynyl-O—. A heteroalkynylene is a divalent heteroalkynyl group.

The term “heteroaryl,” as used herein, refers to an aromatic mono- or polycyclic radical of 5 to 12 atoms having at least one aromatic ring and containing one, two, or three ring heteroatoms selected from N, O, and S, with the remaining ring atoms being C. One or two ring carbon atoms of the heteroaryl group may be replaced with a carbonyl group. Examples of heteroaryl groups are pyridyl, pyrazoyl, benzooxazolyl, benzoimidazolyl, benzothiazolyl, imidazolyl, oxazolyl, and thiazolyl.

The term “heteroarylalkyl,” as used herein, represents an alkyl group substituted with a heteroaryl group. Exemplary unsubstituted heteroarylalkyl groups are from 7 to 30 carbons (e.g., from 7 to 16 or from 7 to 20 carbons, such as C₂-C₉ heteroaryl C₁-C₆ alkylC₂-C₉, C₂-C₉ heteroaryl C₁-C₁₀ alkylC₂-C₉, or C₂-C₉ heteroaryl C₁-C₂₀ alkylC₂-C₉). In some embodiments, the alkyl and the heteroaryl each can be further substituted with 1, 2, 3, or 4 substituent groups as defined herein for the respective groups.

The term “heteroaryloxy”, as used herein, refers to an oxygen atom substituted with a heteroaryl group, as defined herein, e.g., —O-pyridinyl, or —O-thiazolyl.

The term “heterocyclyl,” as used herein, denotes a mono- or polycyclic radical having 3 to 12 atoms having at least one ring containing one, two, three, or four ring heteroatoms selected from N, O or S, where no ring is aromatic. Examples of heterocyclyl groups include, but are not limited to, morpholinyl, thiomorpholinyl, furyl, piperazinyl, piperidinyl, pyranyl, pyrrolidinyl, tetrahydropyranyl, tetrahydrofuranyl, and 1,3-dioxanyl. A heterocyclyl group may be aromatic or non-aromatic. An aromatic heterocyclyl is also referred to as heteroaryl. A polycyclic heterocyclyl may be fused, bridged, or spiro heterocyclyl.

The term “heterocyclylalkyl,” as used herein, represents an alkyl group substituted with a heterocyclyl group. Exemplary unsubstituted heterocyclylalkyl groups are from 7 to 30 carbons (e.g., from 7 to 16 or from 7 to 20 carbons, such as C₂-C₉ heterocyclyl C₁-C₆ alkylC₂-C₉, C₂-C₉ heterocyclyl C₁-C₁₀ alkylC₂-C₉, or C₂-C₉ heterocyclyl C₁-C₂₀ alkylC₂-C₉). In some embodiments, the akyl and the heterocyclyl each can be further substituted with 1, 2, 3, or 4 substituent groups as defined herein for the respective groups.

The term “hydroxyl,” as used herein, represents an —OH group.

The term “N-protecting group,” as used herein, represents those groups intended to protect an amino group against undesirable reactions during synthetic procedures. Commonly used N-protecting groups are disclosed in Greene, “Protective Groups in Organic Synthesis,” 3^{rd} Edition (John Wiley & Sons, New York, 1999). N-protecting groups include acyl, aryloyl, or carbamyl groups such as formyl, acetyl, propionyl, pivaloyl, t-butylacetyl, 2-chloroacetyl, 2-bromoacetyl, trifluoroacetyl, trichloroacetyl, phthalyl, o-nitrophenoxyacetyl, α-chlorobutyryl, benzoyl, 4-chlorobenzoyl, 4-bromobenzoyl, 4-nitrobenzoyl, and chiral auxiliaries such as protected or unprotected D, L or D, L-amino acids such as alanine, leucine, and phenylalanine; sulfonyl-containing groups such as benzenesulfonyl, and p-toluenesulfonyl; carbamate forming groups such as benzyloxycarbonyl, p-chlorobenzyloxycarbonyl, p-methoxybenzyloxycarbonyl, p-nitrobenzyloxycarbonyl, 2-nitrobenzyloxycarbonyl, p-bromobenzyloxycarbonyl, 3,4-dimethoxybenzyloxycarbonyl, 3,5-dimethoxybenzyloxycarbonyl, 2,4-dimethoxybenzyloxycarbonyl, 4-methoxybenzyloxycarbonyl, 2-nitro-4,5-dimethoxybenzyloxycarbonyl, 3,4,5-trimethoxybenzyloxycarbonyl, 1-(p-biphenylyl)-1-methylethoxycarbonyl, α,α-dimethyl-3,5-dimethoxybenzyloxycarbonyl, benzhydryloxy carbonyl, t-butyloxycarbonyl, diisopropylmethoxycarbonyl, isopropyloxycarbonyl, ethoxycarbonyl, methoxycarbonyl, allyloxycarbonyl, 2,2,2,-trichloroethoxycarbonyl, phenoxycarbonyl, 4-nitrophenoxy carbonyl, fluorenyl-9-methoxycarbonyl, cyclopentyloxycarbonyl, adamantyloxycarbonyl, cyclohexyloxycarbonyl, and phenylthiocarbonyl, arylalkyl groups such as benzyl, triphenylmethyl, and benzyloxymethyl, and silyl groups, such as trimethylsilyl. Preferred N-protecting groups are alloc, formyl, acetyl, benzoyl, pivaloyl, t-butylacetyl, alanyl, phenylsulfonyl, benzyl, t-butyloxycarbonyl (Boc), and benzyloxycarbonyl (Cbz).

The term “nitro,” as used herein, represents an NO₂ group.

The term “oxyheteroaryl,” as used herein, represents a heteroaryl group having at least one endocyclic oxygen atom.

The term “oxyheterocyclyl,” as used herein, represents a heterocyclyl group having at least one endocyclic oxygen atom.

The term “thiol,” as used herein, represents an —SH group.

The alkyl, alkenyl, alkynyl, heteroalkyl, heteroalkenyl, heteroalkynyl, carbocyclyl (e.g., cycloalkyl), aryl, heteroaryl, and heterocyclyl groups may be substituted or unsubstituted. When substituted, there will generally be 1 to 4 substituents present, unless otherwise specified. Substituents include, for example: aryl (e.g., substituted and unsubstituted phenyl), carbocyclyl (e.g., substituted and unsubstituted cycloalkyl), cycloalkoxy, halo (e.g., fluoro), heteroaryloxy, hydroxyl, oxo, heteroalkyl (e.g., substituted and unsubstituted methoxy, ethoxy, or thioalkoxy), heteroaryl, heterocyclyl, amino (e.g., NH₂ or mono- or dialkyl amino), azido, cyano, nitro, or thiol. Aryl, carbocyclyl (e.g., cycloalkyl), heteroaryl, and heterocyclyl groups may also be substituted with alkyl (unsubstituted and substituted such as arylalkyl (e.g., substituted and unsubstituted benzyl)).

Compounds of the invention can have one or more asymmetric carbon atoms and can exist in the form of optically pure enantiomers, mixtures of enantiomers such as, for example, racemates, optically pure diastereoisomers, mixtures of diastereoisomers, diastereoisomeric racemates or mixtures of diastereoisomeric racemates. The optically active forms can be obtained, for example, by resolution of the racemates, by asymmetric synthesis or asymmetric chromatography (chromatography with a chiral adsorbent or eluant). That is, certain of the disclosed compounds may exist in various stereoisomeric forms. Stereoisomers are compounds that differ only in their spatial arrangement. Enantiomers are pairs of stereoisomers whose mirror images are not superimposable, most commonly because they contain an asymmetrically substituted carbon atom that acts as a chiral center. “Enantiomer” means one of a pair of molecules that are mirror images of each other and are not superimposable. Diastereomers are stereoisomers that are not related as mirror images, most commonly because they contain two or more asymmetrically substituted carbon atoms and represent the configuration of substituents around one or more chiral carbon atoms. Enantiomers of a compound can be prepared, for example, by separating an enantiomer from a racemate using one or more well-known techniques and methods, such as, for example, chiral chromatography and separation methods based thereon. The appropriate technique and/or method for separating an enantiomer of a compound described herein from a racemic mixture can be readily determined by those of skill in the art. “Racemate” or “racemic mixture” means a compound containing two enantiomers, where such mixtures exhibit no optical activity; i.e., they do not rotate the plane of polarized light. “Geometric isomer” means isomers that differ in the orientation of substituent atoms in relationship to a carbon-carbon double bond, to a cycloalkyl ring, or to a bridged bicyclic system. Atoms (other than H) on each side of a carbon-carbon double bond may be in an E (substituents are on opposite sides of the carbon-carbon double bond) or Z (substituents are oriented on the same side) configuration. “R,” “S,” “S*,” “R*,” “E,” “Z,” “cis,” and “trans,” indicate configurations relative to the core molecule. Certain of the disclosed compounds may exist in atropisomeric forms. Atropisomers are stereoisomers resulting from hindered rotation about single bonds where the steric strain barrier to rotation is high enough to allow for the isolation of the conformers. The compounds of the invention may be prepared as individual isomers by either isomer-specific synthesis or resolved from an isomeric mixture. Conventional resolution techniques include forming the salt of a free base of each isomer of an isomeric pair using an optically active acid (followed by fractional crystallization and regeneration of the free base), forming the salt of the acid form of each isomer of an isomeric pair using an optically active amine (followed by fractional crystallization and regeneration of the free acid), forming an ester or amide of each of the isomers of an isomeric pair using an optically pure acid, amine or alcohol (followed by chromatographic separation and removal of the chiral auxiliary), or resolving an isomeric mixture of either a starting material or a final product using various well known chromatographic methods. When the stereochemistry of a disclosed compound is named or depicted by structure, the named or depicted stereoisomer is at least 60%, 70%, 80%, 90%, 99% or 99.9%) by weight relative to the other stereoisomers. When a single enantiomer is named or depicted by structure, the depicted or named enantiomer is at least 60%, 70%, 80%, 90%, 99% or 99.9% by weight optically pure. When a single diastereomer is named or depicted by structure, the depicted or named diastereomer is at least 60%, 70%, 80%, 90%, 99% or 99.9% by weight pure. Percent optical purity is the ratio of the weight of the enantiomer or over the weight of the enantiomer plus the weight of its optical isomer. Diastereomeric purity by weight is the ratio of the weight of one diastereomer or over the weight of all the diastereomers. When the stereochemistry of a disclosed compound is named or depicted by structure, the named or depicted stereoisomer is at least 60%, 70%, 80%, 90%, 99% or 99.9% by mole fraction pure relative to the other stereoisomers. When a single enantiomer is named or depicted by structure, the depicted or named enantiomer is at least 60%, 70%, 80%, 90%, 99% or 99.9% by mole fraction pure. When a single diastereomer is named or depicted by structure, the depicted or named diastereomer is at least 60%, 70%, 80%, 90%, 99% or 99.9% by mole fraction pure. Percent purity by mole fraction is the ratio of the moles of the enantiomer or over the moles of the enantiomer plus the moles of its optical isomer.

Similarly, percent purity by moles fraction is the ratio of the moles of the diastereomer or over the moles of the diastereomer plus the moles of its isomer. When a disclosed compound is named or depicted by structure without indicating the stereochemistry, and the compound has at least one chiral center, it is to be understood that the name or structure encompasses either enantiomer of the compound free from the corresponding optical isomer, a racemic mixture of the compound or mixtures enriched in one enantiomer relative to its corresponding optical isomer. When a disclosed compound is named or depicted by structure without indicating the stereochemistry and has two or more chiral centers, it is to be understood that the name or structure encompasses a diastereomer free of other diastereomers, a number of diastereomers free from other diastereomeric pairs, mixtures of diastereomers, mixtures of diastereomeric pairs, mixtures of diastereomers in which one diastereomer is enriched relative to the other diastereomer(s) or mixtures of diastereomers in which one or more diastereomer is enriched relative to the other diastereomers. The invention embraces all of these forms.

### Definitions

In this application, unless otherwise clear from context, (i) the term “a” may be understood to mean “at least one”; (ii) the term “or” may be understood to mean “and/or”; (iii) the terms “including” and “including” may be understood to encompass itemized components or steps whether presented by themselves or together with one or more additional components or steps; and (iv) the terms “about” and “approximately” may be understood to permit standard variation as would be understood by those of ordinary skill in the art; and (v) where ranges are provided, endpoints are included.

As used herein, the term “administration” refers to the administration of a composition (e.g., a compound, a complex or a preparation that includes a compound or complex as described herein) to a subject or system. Administration to an animal subject (e.g., to a human) may be by any appropriate route. For example, in some embodiments, administration may be bronchial (including by bronchial instillation), buccal, enteral, interdermal, intra-arterial, intradermal, intragastric, intramedullary, intramuscular, intranasal, intraperitoneal, intrathecal, intravenous, intraventricular, mucosal, nasal, oral, rectal, subcutaneous, sublingual, topical, tracheal (including by intratracheal instillation), transdermal, vaginal and vitreal.

As used herein, the term “animal” refers to any member of the animal kingdom. In some embodiments, “animal” refers to humans, at any stage of development. In some embodiments, “animal” refers to non-human animals, at any stage of development. In some embodiments, the non-human animal is a mammal (e.g., a rodent, a mouse, a rat, a rabbit, a monkey, a dog, a cat, a sheep, cattle, a primate, and/or a pig). In some embodiments, animals include, but are not limited to, mammals, birds, reptiles, amphibians, fish, and/or worms. In some embodiments, an animal may be a transgenic animal, genetically engineered animal, and/or a clone.

As used herein, the terms “approximately” and “about” are each intended to encompass normal statistical variation as would be understood by those of ordinary skill in the art as appropriate to the relevant context. In certain embodiments, the terms “approximately” or “about” each refer to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of a stated value, unless otherwise stated or otherwise evident from the context (e.g., where such number would exceed 100% of a possible value).

Two events or entities are “associated” with one another, as that term is used herein, if the presence, level and/or form of one is correlated with that of the other. For example, a particular entity (e.g., polypeptide) is considered to be associated with a particular disease, disorder, or condition, if its presence, level and/or form correlates with incidence of and/or susceptibility of the disease, disorder, or condition (e.g., across a relevant population).

As used herein, the terms “benefit” and “response” are used interchangeably in the context of a subject, such as a human subject undergoing therapy for the treatment of a neurological disorder, for example, amyotrophic lateral sclerosis, frontotemporal degeneration (also referred to as frontotemporal lobar degeneration and frontotemporal dementia), Alzheimer's disease, Parkinson's disease, dementia with Lewy Bodies, corticobasal degeneration, progressive supranuclear palsy, dementia parkinsonism ALS complex of Guam, Huntington's disease, Inclusion body myopathy with early-onset Paget disease and frontotemporal dementia (IBMPFD), sporadic inclusion body myositis, myofibrillar myopathy, dementia pugilistica, chronic traumatic encephalopathy, Alexander disease, and hereditary inclusion body myopathy. The terms “benefit” and “response” refer to any clinical improvement in the subject's condition. Exemplary benefits in the context of a subject undergoing treatment for a neurological disorder using the compositions and methods described herein (e.g., in the context of a human subject undergoing treatment for a neurological disorder described herein, such as amyotrophic lateral sclerosis, with a FYVE-type zinc finger containing phosphoinositide kinase (PIKfyve) inhibitor described herein, such as an inhibitory small molecule, antibody, antigen-binding fragment thereof, or interfering RNA molecule) include the slowing and halting of disease progression, as well as suppression of one or more symptoms associated with the disease. Particularly, in the context of a patient (e.g., a human patient) undergoing treatment for amyotrophic lateral sclerosis with a compound of the invention, examples of clinical “benefits” and “responses” are (i) an improvement in the subject's condition as assessed using the amyotrophic lateral sclerosis functional rating scale (ALSFRS) or the revised ALSFRS (ALSFRS-R) following administration of the compound of the invention, such as an improvement in the subject's ALSFRS or ALSFRS-R score within one or more days, weeks, or months following administration of the compound of the invention (e.g., an improvement in the subject's ALSFRS or ALSFRS-R score within from about 1 day to about 48 weeks (e.g., within from about 2 days to about 36 weeks, from about 4 weeks to about 24 weeks, from about 8 weeks to about 20 weeks, or from about 12 weeks to about 16 weeks), or more, following the initial administration of the compound of the invention to the subject, such as within 1 day, 2 days, 3 days, 4 days, 5 days, 6 days, 7 days, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, or more, following the initial administration of the compound of the invention to the subject); (ii) an increase in the subject's slow vital capacity following administration of the compound of the invention, such as an increase in the subject's slow vital capacity within one or more days, weeks, or months following administration of the compound of the invention (e.g., an increase in the subject's slow vital capacity within from about 1 day to about 48 weeks (e.g., within from about 2 days to about 36 weeks, from about 4 weeks to about 24 weeks, from about 8 weeks to about 20 weeks, or from about 12 weeks to about 16 weeks), or more, following the initial administration of the compound of the invention to the subject, such as within 1 day, 2 days, 3 days, 4 days, 5 days, 6 days, 7 days, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, or more, following the initial administration of the compound of the invention to the subject); (iii) a reduction in decremental responses exhibited by the subject upon repetitive nerve stimulation, such as a reduction that is observed within one or more days, weeks, or months following administration of the compound of the invention (e.g., a reduction that is observed within from about 1 day to about 48 weeks (e.g., within from about 2 days to about 36 weeks, from about 4 weeks to about 24 weeks, from about 8 weeks to about 20 weeks, or from about 12 weeks to about 16 weeks), or more, following the initial administration of the compound of the invention to the subject, such as within 1 day, 2 days, 3 days, 4 days, 5 days, 6 days, 7 days, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, or more, following the initial administration of the compound of the invention to the subject); (iv) an improvement in the subject's muscle strength, as assessed, for example, by way of the Medical Research Council muscle testing scale (as described, e.g., in Jagtap et al., Ann. Indian. Acad. Neurol. 17:336-339 (2014), the disclosure of which is incorporated herein by reference as it pertains to measuring patient response to neurological disease treatment), such as an improvement that is observed within one or more days, weeks, or months following administration of the compound of the invention (e.g., an improvement that is observed within from about 1 day to about 48 weeks (e.g., within from about 2 days to about 36 weeks, from about 4 weeks to about 24 weeks, from about 8 weeks to about 20 weeks, or from about 12 weeks to about 16 weeks), or more, following the initial administration of the compound of the invention to the subject, such as within 1 day, 2 days, 3 days, 4 days, 5 days, 6 days, 7 days, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, or more, following the initial administration of the compound of the invention to the subject); (v) an improvement in the subject's quality of life, as assessed, for example, using the amyotrophic lateral sclerosis-specific quality of life (ALS-specific QOL) questionnaire, such as an improvement in the subject's quality of life that is observed within one or more days, weeks, or months following administration of the compound of the invention (e.g., an improvement in the subject's quality of life that is observed within from about 1 day to about 48 weeks (e.g., within from about 2 days to about 36 weeks, from about 4 weeks to about 24 weeks, from about 8 weeks to about 20 weeks, or from about 12 weeks to about 16 weeks), or more, following the initial administration of the compound of the invention to the subject, such as within 1 day, 2 days, 3 days, 4 days, 5 days, 6 days, 7 days, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, or more, following the initial administration of the compound of the invention to the subject); and (vi) a decrease in the frequency and/or severity of muscle cramps exhibited by the subject, such as a decrease in cramp frequency and/or severity within one or more days, weeks, or months following administration of the compound of the invention (e.g., a decrease in cramp frequency and/or severity within from about 1 day to about 48 weeks (e.g., within from about 2 days to about 36 weeks, from about 4 weeks to about 24 weeks, from about 8 weeks to about 20 weeks, or from about 12 weeks to about 16 weeks), or more, following the initial administration of the compound of the invention to the subject, such as within 1 day, 2 days, 3 days, 4 days, 5 days, 6 days, 7 days, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, or more, following the initial administration of the compound of the invention to the subject).

As used herein, the term “dosage form” refers to a physically discrete unit of an active compound (e.g., a therapeutic or diagnostic agent) for administration to a subject. Each unit contains a predetermined quantity of active agent. In some embodiments, such quantity is a unit dosage amount (or a whole fraction thereof) appropriate for administration in accordance with a dosing regimen that has been determined to correlate with a desired or beneficial outcome when administered to a relevant population (i.e., with a therapeutic dosing regimen). Those of ordinary skill in the art appreciate that the total amount of a therapeutic composition or compound administered to a particular subject is determined by one or more attending physicians and may involve administration of multiple dosage forms.

As used herein, the term “dosing regimen” refers to a set of unit doses (typically more than one) that are administered individually to a subject, typically separated by periods of time. In some embodiments, a given therapeutic compound has a recommended dosing regimen, which may involve one or more doses. In some embodiments, a dosing regimen includes a plurality of doses each of which are separated from one another by a time period of the same length; in some embodiments, a dosing regimen includes a plurality of doses and at least two different time periods separating individual doses. In some embodiments, all doses within a dosing regimen are of the same unit dose amount. In some embodiments, different doses within a dosing regimen are of different amounts. In some embodiments, a dosing regimen includes a first dose in a first dose amount, followed by one or more additional doses in a second dose amount different from the first dose amount. In some embodiments, a dosing regimen includes a first dose in a first dose amount, followed by one or more additional doses in a second dose amount same as the first dose amount In some embodiments, a dosing regimen is correlated with a desired or beneficial outcome when administered across a relevant population (i.e., is a therapeutic dosing regimen).

In the practice of the methods of the present invention, an “effective amount” of any one of the compounds of the invention or a combination of any of the compounds of the invention or a pharmaceutically acceptable salt thereof, is administered via any of the usual and acceptable methods known in the art, either singly or in combination.

The term “pharmaceutical composition,” as used herein, represents a composition containing a compound described herein formulated with a pharmaceutically acceptable excipient, and manufactured or sold with the approval of a governmental regulatory agency as part of a therapeutic regimen for the treatment of disease in a mammal. Pharmaceutical compositions can be formulated, for example, for oral administration in unit dosage form (e.g., a tablet, capsule, caplet, gelcap, or syrup); for topical administration (e.g., as a cream, gel, lotion, or ointment); for intravenous administration (e.g., as a sterile solution free of particulate emboli and in a solvent system suitable for intravenous use); or in any other pharmaceutically acceptable formulation.

A “pharmaceutically acceptable excipient,” as used herein, refers any ingredient other than the compounds described herein (for example, a vehicle capable of suspending or dissolving the active compound) and having the properties of being substantially nontoxic and non-inflammatory in a patient. Excipients may include, for example, antiadherents, antioxidants, binders, coatings, compression aids, disintegrants, dyes (colors), emollients, emulsifiers, fillers (diluents), film formers or coatings, flavors, fragrances, glidants (flow enhancers), lubricants, preservatives, printing inks, sorbents, suspensing or dispersing agents, sweeteners, and waters of hydration. Exemplary excipients include, but are not limited to: butylated hydroxytoluene (BHT), calcium carbonate, calcium phosphate (dibasic), calcium stearate, croscarmellose, crosslinked polyvinyl pyrrolidone, citric acid, crospovidone, cysteine, ethylcellulose, gelatin, hydroxypropyl cellulose, hydroxypropyl methylcellulose, lactose, magnesium stearate, maltitol, mannitol, methionine, methylcellulose, methyl paraben, microcrystalline cellulose, polyethylene glycol, polyvinyl pyrrolidone, povidone, pregelatinized starch, propyl paraben, retinyl palmitate, shellac, silicon dioxide, sodium carboxymethyl cellulose, sodium citrate, sodium starch glycolate, sorbitol, starch (corn), stearic acid, sucrose, talc, titanium dioxide, vitamin A, vitamin E, vitamin C, and xylitol.

As used herein, the term “pharmaceutically acceptable salt” means any pharmaceutically acceptable salt of the compound of formula (I). For example, pharmaceutically acceptable salts of any of the compounds described herein include those that are within the scope of sound medical judgment, suitable for use in contact with the tissues of humans and animals without undue toxicity, irritation, allergic response and are commensurate with a reasonable benefit/risk ratio. Pharmaceutically acceptable salts are well known in the art. For example, pharmaceutically acceptable salts are described in: Berge et al., J. Pharmaceutical Sciences 66:1-19, 1977 and in Pharmaceutical Salts: Properties, Selection, and Use, (Eds. P. H. Stahl and C. G. Wermuth), Wiley-VCH, 2008. The salts can be prepared in situ during the final isolation and purification of the compounds described herein or separately by reacting a free base group with a suitable organic acid.

The compounds of the invention may have ionizable groups so as to be capable of preparation as pharmaceutically acceptable salts. These salts may be acid addition salts involving inorganic or organic acids or the salts may, in the case of acidic forms of the compounds of the invention be prepared from inorganic or organic bases. Frequently, the compounds are prepared or used as pharmaceutically acceptable salts prepared as addition products of pharmaceutically acceptable acids or bases. Suitable pharmaceutically acceptable acids and bases and methods for preparation of the appropriate salts are well-known in the art. Salts may be prepared from pharmaceutically acceptable non-toxic acids and bases including inorganic and organic acids and bases.

The terms “PIKfyve” and “FYVE-type zinc finger containing phosphoinositide kinase” are used interchangeably herein and refer to the enzyme that catalyzes phosphorylation of phosphatidylinositol 3-phosphate to produce phosphatidylinositol 3,5-bisphosphate, for example, in human subjects. The terms “PIKfyve” and “FYVE-type zinc finger containing phosphoinositide kinase” refer not only to wild-type forms of PIKfyve, but also to variants of wild-type PIKfyve proteins and nucleic acids encoding the same. The gene encoding PIKfyve can be accessed under NCBI Reference Sequence No. NG_021188.1. Exemplary transcript sequences of wild-type form of human PIKfyve can be accessed under NCBI Reference Sequence Nos. NM_015040.4, NM_152671.3, and NM_001178000.1. Exemplary protein sequences of wild-type form of human PIKfyve can be accessed under NCBI Reference Sequence Nos. NP_055855.2, NP_689884.1, and NP_001171471.1.

As used herein, the term “PIKfyve inhibitor” refers to substances, such as compounds of Formula 1. Inhibitors of this type may, for example, competitively inhibit PIKfyve activity by specifically binding the PIKfyve enzyme (e.g., by virtue of the affinity of the inhibitor for the PIKfyve active site), thereby precluding, hindering, or halting the entry of one or more endogenous substrates of PIKfyve into the enzyme's active site. Additional examples of PIKfyve inhibitors that suppress the activity of the PIKfyve enzyme include substances that may bind PIKfyve at a site distal from the active site and attenuate the binding of endogenous substrates to the PIKfyve active site by way of a change in the enzyme's spatial conformation upon binding of the inhibitor. In addition to encompassing substances that modulate PIKfyve activity, the term “PIKfyve inhibitor” refers to substances that reduce the concentration and/or stability of PIKfyve mRNA transcripts in vivo, as well as those that suppress the translation of functional PIKfyve enzyme.

The term “pure” means substantially pure or free of unwanted components (e.g., other compounds and/or other components of a cell lysate), material defilement, admixture or imperfection.

Representative acid addition salts include acetate, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, and valerate salts. Representative alkali or alkaline earth metal salts include sodium, lithium, potassium, calcium, and magnesium, as well as nontoxic ammonium, quaternary ammonium, and amine cations, including, but not limited to ammonium, tetramethylammonium, tetraethylammonium, methylamine, dimethylamine, trimethylamine, triethylamine, and ethylamine.

A variety of clinical indicators can be used to identify a patient as “at risk” of developing a particular neurological disease. Examples of patients (e.g., human patients) that are “at risk” of developing a neurological disease, such as amyotrophic lateral sclerosis, frontotemporal degeneration, Alzheimer's disease, Parkinson's disease, dementia with Lewy Bodies, corticobasal degeneration, progressive supranuclear palsy, dementia parkinsonism ALS complex of Guam, Huntington's disease, Inclusion body myopathy with early-onset Paget disease and frontotemporal dementia (IBMPFD), sporadic inclusion body myositis, myofibrillar myopathy, dementia pugilistica, chronic traumatic encephalopathy, Alexander disease, and hereditary inclusion body myopathy, include (i) subjects exhibiting or prone to exhibit aggregation of TAR-DNA binding protein (TDP)-43, and (ii) subjects expressing a mutant form of TDP-43 containing a mutation associated with TDP-43 aggregation and toxicity, such as a mutation selected from Q³³¹K, M337V, Q³⁴³R, N345K, R361 S, and N390D. Subjects that are “at risk” of developing amyotrophic lateral sclerosis may exhibit one or both of these characteristics, for example, prior to the first administration of a PIKfyve inhibitor in accordance with the compositions and methods described herein.

As used herein, the terms “TAR-DNA binding protein-43” and “TDP-43” are used interchangeably and refer to the transcription repressor protein involved in modulating HIV-1 transcription and alternative splicing of the cystic fibrosis transmembrane conductance regulator (CFTR) pre-mRNA transcript, for example, in human subjects. The terms “TAR-DNA binding protein-43” and “TDP-43” refer not only to wild-type forms of TDP-43, but also to variants of wild-type TDP-43 proteins and nucleic acids encoding the same. The amino acid sequence and corresponding mRNA sequence of a wild-type form of human TDP-43 are provided under NCBI Reference Sequence Nos. NM_007375.3 and NP_031401.1, respectively.

The terms “TAR-DNA binding protein-43” and “TDP-43” as used herein include, for example, forms of the human TDP-43 protein that have an amino acid sequence that is at least 85% identical to the amino acid sequence of NCBI Reference Sequence No. NP_031401.1 (e.g., 85%, 86%, 87%, 88%, 89%, 90%, 91%, 92%, 93%, 94%, 95%, 96%, 97%, 98%, 99%, 99.9%, or 100% identical to the amino acid sequence of NCBI Reference Sequence No. NP_031401.1) and/or forms of the human TDP-43 protein that contain one or more substitutions, insertions, and/or deletions (e.g., one or more conservative and/or nonconservative amino acid substitutions, such as up to 5, 10, 15, 20, 25, or more, conservative or nonconservative amino acid substitutions) relative to a wild-type TDP-43 protein. For instance, patients that may be treated for a neurological disorder as described herein, such as amyotrophic lateral sclerosis, frontotemporal degeneration, Alzheimer's disease, Parkinson's disease, dementia with Lewy Bodies, corticobasal degeneration, progressive supranuclear palsy, dementia parkinsonism ALS complex of Guam, Huntington's disease, Inclusion body myopathy with early-onset Paget disease and frontotemporal dementia (IBMPFD), sporadic inclusion body myositis, myofibrillar myopathy, dementia pugilistica, chronic traumatic encephalopathy, Alexander disease, and hereditary inclusion body myopathy, include human patients that express a form of TDP-43 having a mutation associated with elevated TDP-43 aggregation and toxicity, such as a mutation selected from Q331K, M337V, Q343R, N345K, R361S, and N390D. Similarly, the terms “TAR-DNA binding protein-43” and “TDP-43” as used herein include, for example, forms of the human TDP-43 gene that encode an mRNA transcript having a nucleic acid sequence that is at least 85% identical to the nucleic acid sequence of NCBI Reference Sequence No. NM_007375.3 (e.g., 85%, 86%, 87%, 88%, 89%, 90%, 91%, 92%, 93%, 94%, 95%, 96%, 97%, 98%, 99%, 99.9%, or 100% identical to the amino acid sequence of NCBI Reference Sequence No. NM_007375.3).

As used herein, the term “subject” refers to any organism to which a composition in accordance with the invention may be administered, e.g., for experimental, diagnostic, prophylactic, and/or therapeutic purposes. Typical subjects include any animal (e.g., mammals such as mice, rats, rabbits, non-human primates, and humans). A subject may seek or be in need of treatment, require treatment, be receiving treatment, be receiving treatment in the future, or be a human or animal who is under care by a trained professional for a particular disease or condition.

A “therapeutic regimen” refers to a dosing regimen whose administration across a relevant population is correlated with a desired or beneficial therapeutic outcome.

The term “therapeutically effective amount” means an amount that is sufficient, when administered to a population suffering from or susceptible to a disease, disorder, and/or condition in accordance with a therapeutic dosing regimen, to treat the disease, disorder, and/or condition. In some embodiments, a therapeutically effective amount is one that reduces the incidence and/or severity of, and/or delays onset of, one or more symptoms of the disease, disorder, and/or condition. Those of ordinary skill in the art will appreciate that the term “therapeutically effective amount” does not in fact require successful treatment be achieved in a particular individual. Rather, a therapeutically effective amount may be that amount that provides a particular desired pharmacological response in a significant number of subjects when administered to patients in need of such treatment. It is specifically understood that particular subjects may, in fact, be “refractory” to a “therapeutically effective amount.” To give but one example, a refractory subject may have a low bioavailability such that clinical efficacy is not obtainable. In some embodiments, reference to a therapeutically effective amount may be a reference to an amount as measured in one or more specific tissues (e.g., a tissue affected by the disease, disorder or condition) or fluids (e.g., blood, saliva, serum, sweat, tears, urine, etc). Those of ordinary skill in the art will appreciate that, in some embodiments, a therapeutically effective amount may be formulated and/or administered in a single dose. In some embodiments, a therapeutically effective amount may be formulated and/or administered in a plurality of doses, for example, as part of a dosing regimen.

### BRIEF DESCRIPTION OF THE DRAWINGS

FIG. 1 is a scheme showing an approach to generation of a control TDP-43 yeast model (FAB1 TDP-43). A control yeast TDP-43 model was generated by integrating the human TDP-43 gene and the GAL1 promoter into the yeast genome. The yeast ortholog of human PIKFYVE is FAB1.

FIG. 2 is a scheme showing an approach to generation of a humanized PIKFYVE TDP-43 yeast model (PIKFYVE TDP-43). FAB1 gene through homologous recombination with a G418 resistance cassette (fab1::G418^{R}) (FIG. 2). PIKFYVE was cloned downstream of the GPD promoter harbored on a URA3-containing plasmid and introduced into the fab1::G418R ura3 strain. The pGAL1-TDP-43 construct was then introduced into the “humanized” yeast strain and assessed for cytotoxicity.

FIG. 3 is a histogram generated from the flow cytometry-based viability assay of FAB1 TDP-43.

FIG. 4 is a histogram generated from the flow cytometry-based viability assay of PIKFYVE TDP-43. Upon induction of TDP-43, there was a marked increase in inviable cells (rightmost population), with a more pronounced effect in PIKFYVE TDP-43 than in FAB1 TDP-43 strain (see FIG. 3).

FIG. 5 is an overlay of histograms generated from the flow cytometry-based viability assay of FAB1 TDP-43 in the presence of APY0201.

FIG. 6 is an overlay of histograms generated from the flow cytometry-based viability assay of PIKFYVE TDP-43 in the presence of APY0201.

FIG. 7 is a scatter plot comparing cytoprotection efficacy in PIKFYVE TDP-43 to PIKfyve inhibitory activity of test compounds.

### DETAILED DESCRIPTION

The present invention features compositions and methods for treating neurological disorders, such as amyotrophic lateral sclerosis and other neuromuscular disorders, as well as frontotemporal degeneration, Alzheimer's disease, Parkinson's disease, dementia with Lewy Bodies, corticobasal degeneration, progressive supranuclear palsy, dementia parkinsonism ALS complex of Guam, Huntington's disease, Inclusion body myopathy with early-onset Paget disease and frontotemporal dementia (IBMPFD), sporadic inclusion body myositis, myofibrillar myopathy, dementia pugilistica, chronic traumatic encephalopathy, Alexander disease, and hereditary inclusion body myopathy among others. Particularly, the invention provides inhibitors of FYVE-type zinc finger containing phosphoinositide kinase (PIKfyve), that may be administered to a patient (e.g., a human patient) so as to treat or prevent a neurological disorder, such as one or more of the foregoing conditions. In the context of therapeutic treatment, the PIKfyve inhibitor may be administered to the patient to alleviate one or more symptoms of the disorder and/or to remedy an underlying molecular pathology associated with the disease, such as to suppress or prevent aggregation of TAR-DNA binding protein (TDP)-43.

The disclosure herein is based, in part, on the discovery that PIKfyve inhibition modulates TDP-43 aggregation in cells. Suppression of TDP-43 aggregation exerts beneficial effects in patients suffering from a neurological disorder. Many pathological conditions have been correlated with TDP-43-promoted aggregation and toxicity, such as amyotrophic lateral sclerosis, frontotemporal degeneration, Alzheimer's disease, Parkinson's disease, dementia with Lewy Bodies, corticobasal degeneration, progressive supranuclear palsy, dementia parkinsonism ALS complex of Guam, Huntington's disease, IBMPFD, sporadic inclusion body myositis, myofibrillar myopathy, dementia pugilistica, chronic traumatic encephalopathy, Alexander disease, and hereditary inclusion body myopathy. Without being limited by mechanism, by administering an inhibitor of PIKfyve, patients suffering from diseases associated with TDP-43 aggregation and toxicity may be treated, for example, due to the suppression of TDP-43 aggregation induced by the PIKfyve inhibitor.

Patients that are likely to respond to PIKfyve inhibition as described herein include those that have or are at risk of developing TDP-43 aggregation, such as those that express a mutant form of TDP-43 associated with TDP-43 aggregation and toxicity in vivo. Examples of such mutations in TDP-43 that have been correlated with elevated TDP-43 aggregation and toxicity include Q331 K, M337V, Q343R, N345K, R361S, and N390D, among others. The compositions and methods described herein thus provide the additional clinical benefit of enabling the identification of patients that are likely to respond to PIKfyve inhibitor therapy, as well as processes for treating these patients accordingly.

The sections that follow provide a description of exemplary PIKfyve inhibitors that may be used in conjunction with the compositions and methods disclosed herein. The sections below additionally provide a description of various exemplary routes of administration and pharmaceutical compositions that may be used for delivery of these substances for the treatment of a neurological disorder.

### PIKfyve Inhibitors

PIKfyve inhibitors described herein include compounds of formula 1:

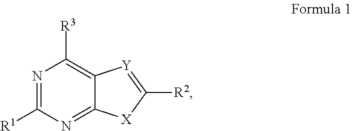

or a pharmaceutically acceptable salt thereof,
where
X is NR^{A}.
Y is CR^{A} or N;
R¹ is optionally substituted C₁-C₁₀ heteroaryl including a 5-membered ring having a nitrogen atom at position 2 relative to the bond to the core; 4,5-dihydropyrazol-1-yl substituted with phenyl; optionally substituted pyrimidin-2-yl, optionally substituted pyridazin-6-yl, optionally substituted pyrimidin-4-yl; pyridin-3-yl optionally substituted with methoxy; optionally substituted indazol-1-yl; optionally substituted indazol-2-yl; optionally substituted indazol-7-yl; optionally substituted isoindolin-6-yl; optionally substituted pyridazin-5-yl; optionally substituted pyrrolidine-1-yl; optionally substituted pyrimidin-6-yl; optionally substituted piperazinyl; phenyl substituted with methoxy, optionally substituted C₁-C₆ alkyl, hydroxyl, optionally substituted C₂-C₉ heteroaryl, optionally substituted C₆-C₁₀ aryl, optionally substituted C₂-C₉ heterocyclyl, or C₃-C₈ cycloalkoxy; optionally substituted C₃ carbocyclyl; optionally substituted morpholin-1-yl; optionally substituted benzodioxolyl; optionally substituted benzopyrrolidonyl; optionally substituted tetrahydroquinoline; optionally substituted monoalkylamino; optionally substituted dialkylamino; amino monosubstituted with optionally substituted C₂-C₉ heteroaryl; halo; optionally substituted C₂-C₉ heterocycle C₁ alkyl; optionally substituted C₂-C₉ heteroaryl C₁ alkyl; optionally substituted benzodioxanyl; —NHNHR^{1A}; —N(R^{1A})N═C(R^{1B})₂; —C(R^{1A})═N—N(R^{1B})₂; —C(R^{1A})═NOR^{1A}; or -Q¹-N(R^{1C})₂;
Q¹ is a bond, CH₂, or CO;
each R^{1A} is independently H, optionally substituted C₁-C₆ alkyl, optionally substituted C₆-C₁₀ aryl, or optionally substituted C₆-C₁₀ aryl C₁-C₆ alkyl;
one R^{1B} is independently H, optionally substituted C₁-C₆ alkyl, optionally substituted C₆-C₁₀ aryl, or optionally substituted C₂-C₉ heteroaryl; and the remaining R^{1B} is optionally substituted C₁-C₆ alkyl, optionally substituted C₆-C₁₀ aryl, or optionally substituted C₂-C₉ heteroaryl;
each R^{1C} is independently H, optionally substituted C₁-C₆ alkyl, optionally substituted C₆-C₁₀ aryl, optionally substituted C₃-C₈ cycloalkyl, or optionally substituted C₂-C₉ heteroaryl; or both Ric, together with the nitrogen atom to which they are attached, combine to form C₂-C₉ heterocyclyl or C₂-C₉ heteroaryl; R² is H, halogen, optionally substituted C₆-C₁₀ aryl; optionally substituted C₁₋₉ heterocyclyl; —O-pyridin-3-yl; optionally substituted C₃-C₈ cycloalkyl; optionally substituted C₃-C₈ cycloalkenyl, C₁-C₂ alkyl optionally substituted with hydroxy, methoxy, —CH₂OH, pyridin-4-yl, 4-pyridon-1-yl, —O-pyridin-4-yl, oxo, or dialkyl amino; C₁ alkyl optionally substituted with deuterium, oxo, hydroxy, halo, or amino substituted with C₃ cycloalkyl; C₃ alkyl substituted with hydroxy, oxo, or dialkyl amino; C₄ alkyl; optionally substituted C₂-C₉ heteroaryl; -Q-N(R^{1c})₂; —S(O)ᵣ—R^{1A}; or —P(O)(R^{1A})₂; and each R^{A} is independently H, C₁-C₂ alkyl optionally substituted with hydroxyl or —S(O)₁— (optionally substituted C₁-C₆ alkyl), C₃ alkyl, C₄-C₅ alkyl substituted with hydroxyl, optionally substituted C₂-C₉ heteroaryl C₁-C₆ alkyl; optionally substituted C₃-C₈ cycloalkyl, optionally substituted C₃-C₈ cycloalkyl C₁-C₆ alkyl, optionally substituted C₆-C₁₀ aryl, or optionally substituted C₂-C₉ heteroaryl; or R² and R^{A}, together with the atoms to which they are attached, combine to form an optionally substituted C₃-C₄ heterocyclic ring, and the remaining R^{A}, if present, is H, C₁-C₂ alkyl optionally substituted with hydroxyl or —S(O)₁— (optionally substituted C₁-C₆ alkyl), C₃ alkyl, C₄-C₅ alkyl substituted with hydroxyl, optionally substituted C₂-C₉ heteroaryl C₁-C₆ alkyl; optionally substituted C₃-C₈ cycloalkyl, optionally substituted C₃-C₈ cycloalkyl C₁-C₆ alkyl, optionally substituted C₆-C₁₀ aryl, or optionally substituted C₂-C₉ heteroaryl;
R is 0, 1, or 2; and
R³ is

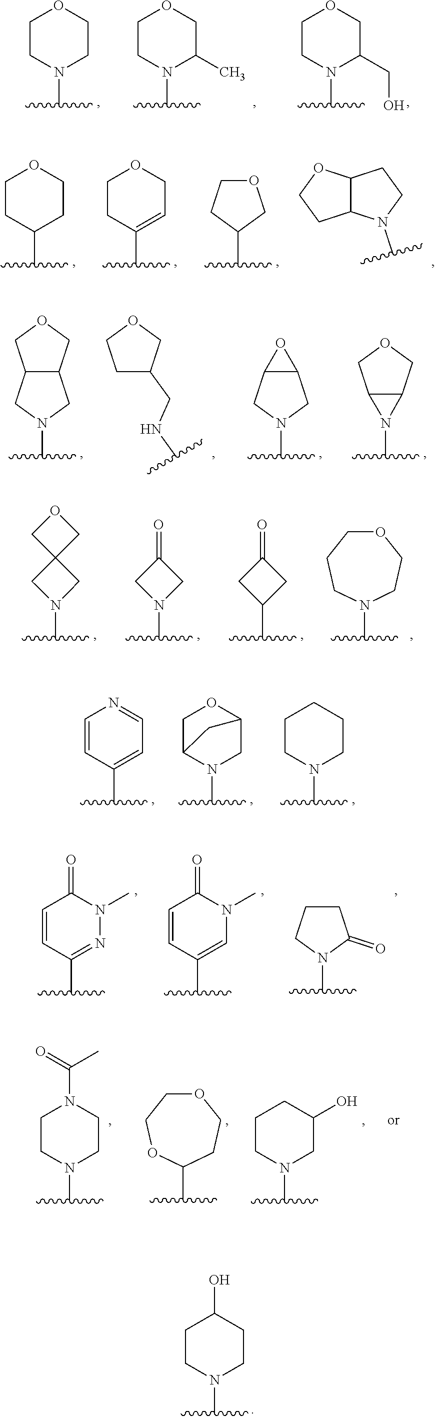

PIKfyve inhibitors described herein also include compounds of formula 2:

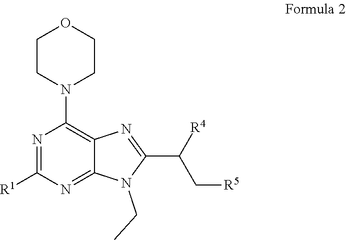

or a pharmaceutically acceptable salt thereof,
where R¹ is optionally substituted pyrazol-1-yl, phenyl substituted with optionally substituted C₂-C₉ heteroaryl, or optionally substituted pyridimin-4-yl; and
R⁴ and R⁵ are each, independently, hydroxyl or methoxy.
PIKfyve inhibitors described herein also include compounds of formula 3:

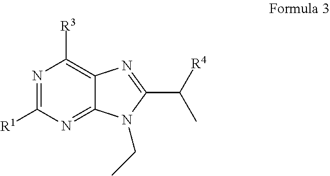

or a pharmaceutically acceptable salt thereof,
where R¹ is optionally substituted pyrazol-1-yl, phenyl substituted with optionally substituted heteroaryl, optionally substituted indazol-1-yl, or optionally substituted indazol-2-yl; R⁴ is hydroxyl, 4-pyridinon-1-yl, —O-pyridin-3-yl, or CH₂OH; and
R³ is pyridin-4-yl or morpholin-1-yl.

PIKfyve inhibitors described herein also include compounds of formula 4:

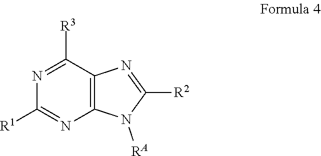

or a pharmaceutically acceptable salt thereof,
where R¹ is phenyl optionally substituted with methoxy or optionally substituted heteroaryl or optionally substituted pyrazol-1-yl,
R³ is morpholin-1-yl or piperidin-1-yl; and

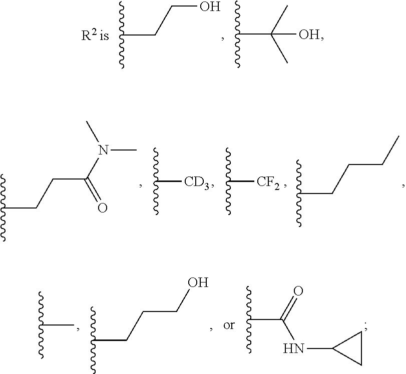

and

R^{A} is ethyl, 2-hydroxy-ethyl, or

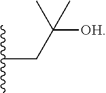 [SMILES: *CC(C)(C)O]

PIKfyve inhibitors described herein also include compounds of formula 5:

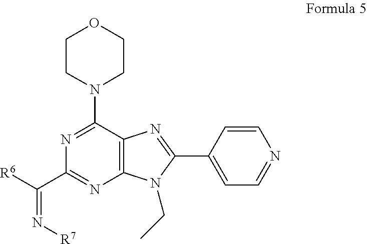

or a pharmaceutically acceptable salt thereof,
where R⁶ is hydrogen or methyl; and
R⁷ is optionally substituted phenoxy, optionally substituted benzyloxy, or optionally substituted amine.

PIKfyve inhibitors described herein also include compounds of formula 6:

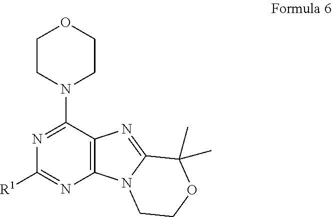

or a pharmaceutically acceptable salt thereof,
where R¹ is optionally substituted pyrazol-1-yl or —N(R^{1A})N═C(R^{1B})₂.

PIKfyve inhibitors described herein also include compounds of formula 7:

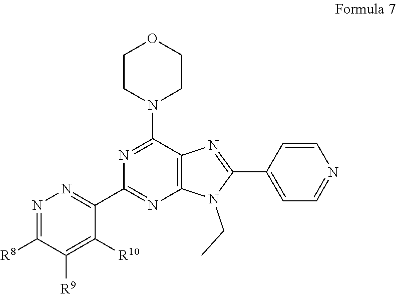

or a pharmaceutically acceptable salt thereof,
where R⁸ is hydrogen or methoxy;
R⁹ is hydrogen or phenyl; and
R¹⁰ is hydrogen or phenyl.

PIKfyve inhibitors described herein also include compounds of formula 8:

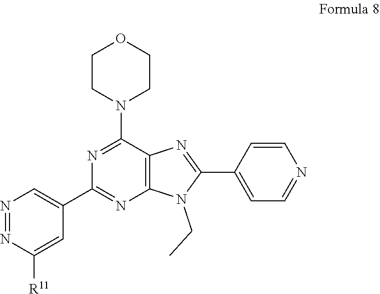

or a pharmaceutically acceptable salt thereof,
where R¹¹ is hydrogen or phenyl.

PIKfyve inhibitors described herein also include compounds of formula 9:

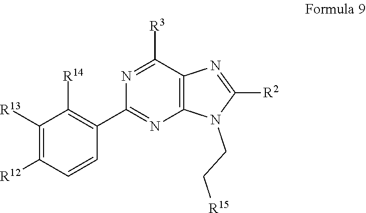

or a pharmaceutically acceptable salt thereof,
where R¹² is hydrogen, methoxy, or CH₂OH;
R¹³ is hydrogen, methoxy, C₃ cycloalkoxy, optionally substituted C₂-C₉ heteroaryl, optionally substituted C₂-C₉ heterocyclyl, or optionally substituted C₁-C₆ alkyl;
R¹⁴ is hydrogen or C₃ cycloalkoxy, or optionally substituted C₂-C₉ heterolaryl;
R¹⁵ is hydrogen or hydroxyl;
R² is hydrogen, pyridin-4-yl,

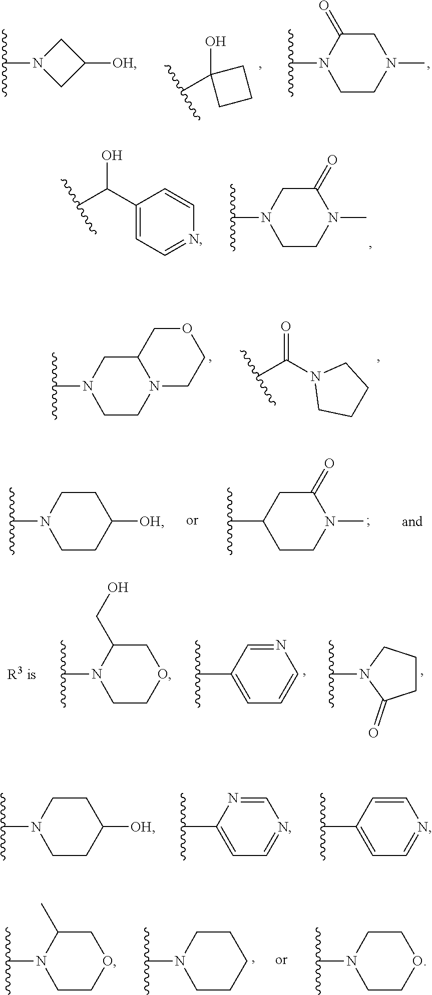

PIKfyve inhibitors described herein also include compounds of formula 10:

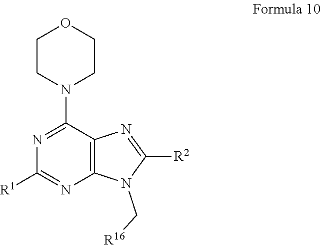

or a pharmaceutically acceptable salt thereof,
where R¹ is

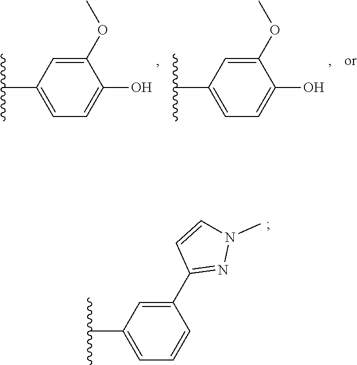

R¹⁶ is hydrogen or pyridine-3-yl; and
R² is pyridin-4-yl or hydrogen.

PIKfyve inhibitors described herein also include compounds of formula 11:

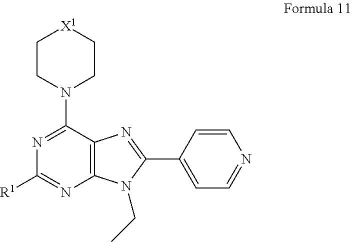

or a pharmaceutically acceptable salt thereof,
where X¹ is O or CH₂; and
R¹ is —N(R^{1A})N═C(R^{1B})₂.

PIKfyve inhibitors described herein also include compounds of formula 12:

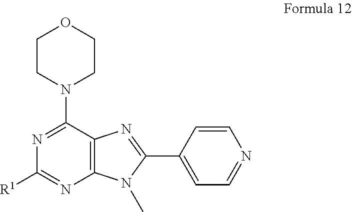

or a pharmaceutically acceptable salt thereof,
where R¹ is

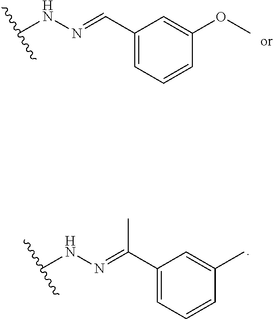 [SMILES: *N/N=C/c1cccc(OC)c1 ; *N/N=C(\C)c1cccc(C)c1]

Exemplary PIKfyve inhibitors described herein also include compounds of formula 13:

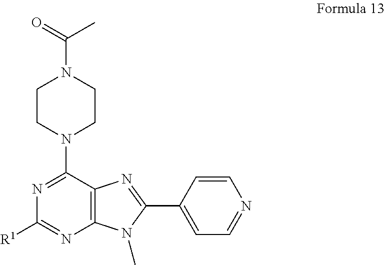

or a pharmaceutically acceptable salt thereof,
where R¹ is —N(R^{1A})N═C(R^{1B})₂.

PIKfyve inhibitors described herein also include compounds of formula 14:

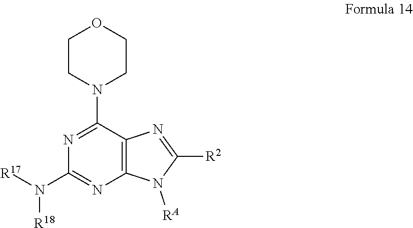

or a pharmaceutically acceptable salt thereof,
where R¹⁷ is optionally substituted C₆-C₁₀ aryl C₁-C₆ alkyl; optionally substituted C₆-C₁₀ heteroaryl C₁-C₆ alkyl; —NH₂, optionally substituted C₃-C₈ cycloalkyl; or optionally substituted C₂-C₉ heterlaryl;
R¹⁸ is hydrogen or optionally substituted C₁-C₆ alkyl;
R^{A} is methyl or ethyl; and
R² is pyridin-4-yl or hydrogen.

PIKfyve inhibitors described herein also include compounds of formula 15:

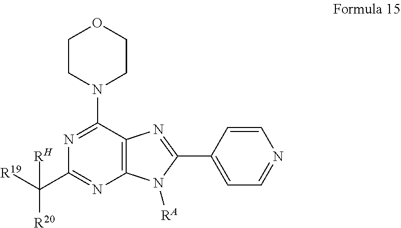

or a pharmaceutically acceptable salt thereof,
where R¹⁹ is optionally substituted amino, optionally substituted C₂-C₉ heterocycle, optionally substituted C₂-C₉ heteroaryl;
R^{H} and R²⁰, together with the atom to which they are attached, combine to form oxo;
R²⁰ is hydrogen or R²⁰ and R^{H}, together with the atom to which they are attached, combine to form oxo; and
PIKfyve inhibitors described herein also include compounds of formula 16:

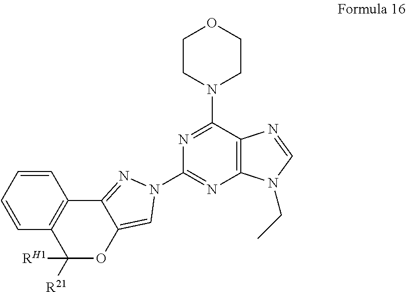

or a pharmaceutically acceptable salt thereof,
where R²¹ is hydrogen or R²¹ and R^{H1}, together with the atom to which they are attached, combine to form oxo; and
R^{H1} is hydrogen or R^{H}1 and R²¹, together with the atom to which they are attached, combine to form oxo.

PIKfyve inhibitors described herein also include compounds of formula 17:

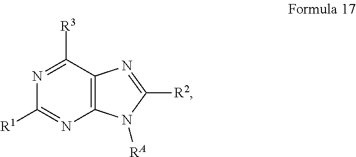

or a pharmaceutically acceptable salt thereof,
where R¹ is pyrazol-1-yl disubstituted with optionally substituted C₆-C₁₀ aryl; optionally substituted C₁-C₆ heteroalkyl; optionally substituted C₁-C₆ alkyl; optionally substituted C₂-C₉ heteroaryl, halo, hydroxy, optionally substituted C₃-C₈ cycloalkyl, or optionally substituted C₁-C₆ alkyl;
R³ is

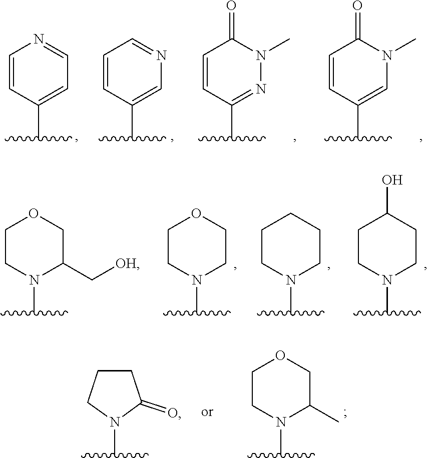

R^{A} is ethyl, 2-hydroxy-ethyl, methyl,

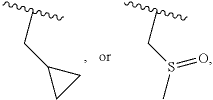

and R² is hydrogen, methyl, ethyl, halo, pyridin-3-yl, pyridin-4-yl, cyclopropyl,

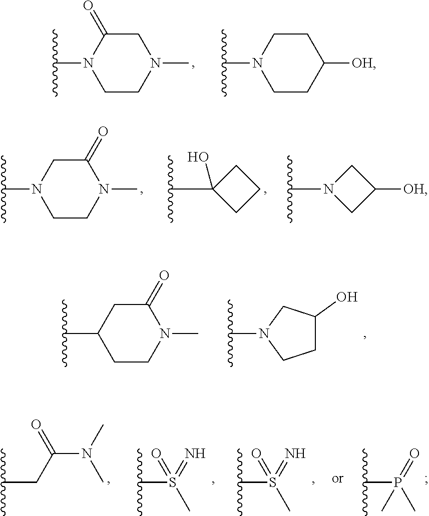

or R² and R^{A}, together with the atoms to which they are attached, combine to form an optionally substituted C₄ heterocyclyl.

PIKfyve inhibitors described herein also include compounds of formula 18:

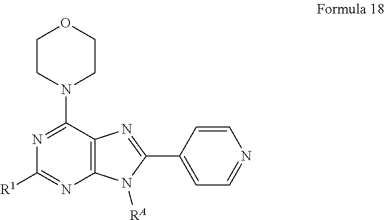

or a pharmaceutically acceptable salt thereof,
where R¹ is optionally substituted triazolyl; and
R^{A} is methyl, ethyl, or cyclopropyl.

PIKfyve inhibitors described herein also include compounds of formula 19:

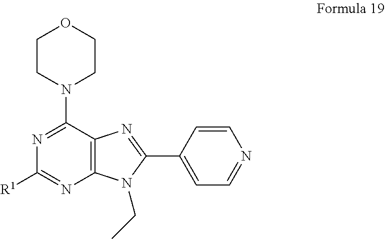

or a pharmaceutically acceptable salt thereof,
where R¹ is optionally substituted indazolyl or optionally substituted 4,5,6,7-tetrahydrotriazaindenyl.

PIKfyve inhibitors described herein also include compounds of formula 20:

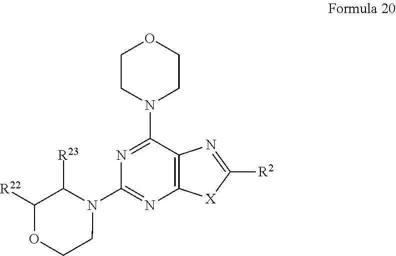

or a pharmaceutically acceptable salt thereof,
where X is S or NR^{A}
R²² is hydrogen or phenyl;
R²³ is hydrogen or methyl;
R² is pyrazol-3-yl, pyridine-4-yl, or 4-phenyl-pyrazol-1-yl; and
R^{A} is methyl.

PIKfyve inhibitors described herein also include compounds of formula 21:

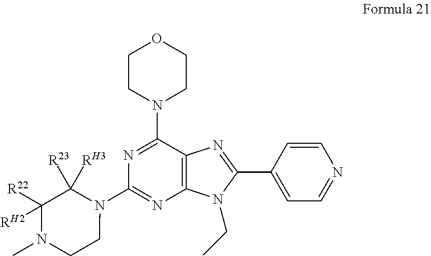

or a pharmaceutically acceptable salt thereof,
where R²² is phenyl, pyridine-2-yl, or R²² and R^{H2} together with the atom to which they are attached, combine to form oxo;
R^{H2} is hydrogen or R^{H2} and R²² together with the atom to which they are attached, combine to form oxo;
R²³ is hydrogen or R²³ and R^{H3}, together with the atom to which they are attached, combine to form oxo; and
R^{H3} is hydrogen or R^{H3} and R²³, together with the atom to which they are attached, combine to form oxo.

PIKfyve inhibitors described herein also include compounds of formula 22:

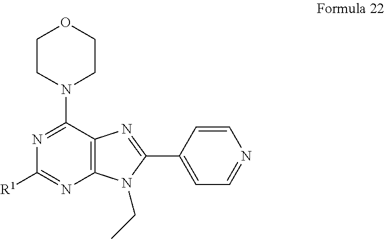

or a pharmaceutically acceptable salt thereof,
where R¹ is

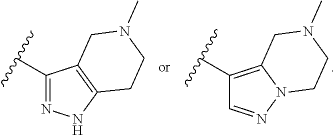 [SMILES: *c1n[nH]c2c1CN(C)CC2 ; *c1cnn2c1CN(C)CC2]

PIKfyve inhibitors described herein also include compounds of formula 23:

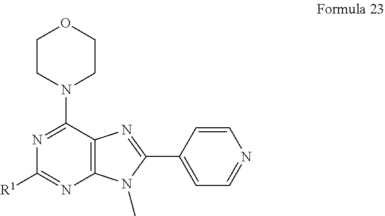

or a pharmaceutically acceptable salt thereof,
where R¹ is

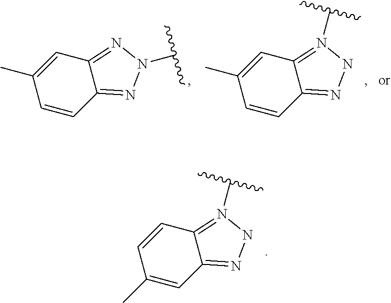

PIKfyve inhibitors described herein also include compounds of formula 24:

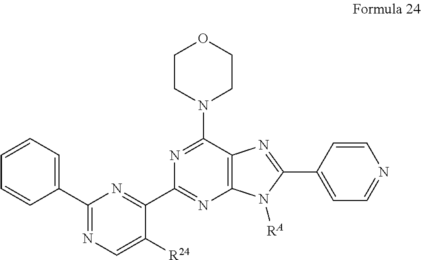

or a pharmaceutically acceptable salt thereof,
where R²⁴ is methoxy, methyl or hydroxyl; and
R^{A} is methyl or ethyl.
PIKfyve inhibitors described herein also include compounds of formula 25:

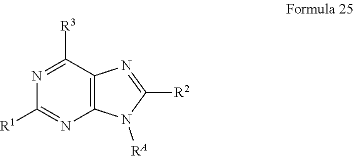

or a pharmaceutically acceptable salt thereof,
where R¹ is optionally substituted pyrazolyl, optionally substituted pyrimidin-3-yl, or optionally substituted pyridin-4-yl;
R^{A} is methyl or ethyl;
R² is optionally substituted C₂-C₉ heteroaryl, or optionally substituted C₁-C₉ heterocyclyl; and
R³ is

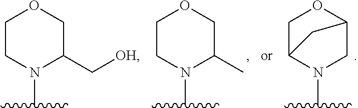

PIKfyve inhibitors described herein also include compounds of formula 26:

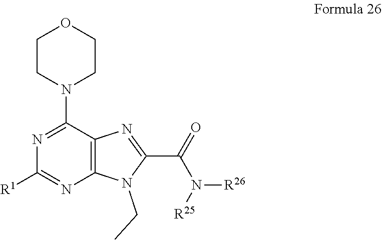

or a pharmaceutically acceptable salt thereof,
where R¹ is optionally substituted pyrazol-1-yl or phenyl substituted with optionally substituted C₂-C₉ heteroaryl; and
R²⁵ and R²⁶, together the atom to which they are attached, combine to form a C₃-C₅ heterocyclyl substituted with hydroxyl.

PIKfyve inhibitors described herein also include compounds of formula 27:

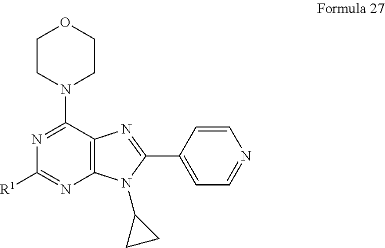

or a pharmaceutically acceptable salt thereof,
where R¹ is optionally substituted pyrazol-1-yl, optionally substituted pyrazol-5-yl, or phenyl substituted with methoxy or C₃-C₈ cycloalkoxy.

PIKfyve inhibitors described herein also include compounds of formula 28:

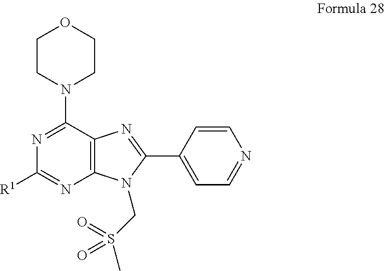

or a pharmaceutically acceptable salt thereof,
where R¹ is optionally substituted pyrazol-1-yl.

PIKfyve inhibitors described herein also include compounds of formula 29:

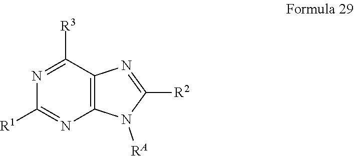

or a pharmaceutically acceptable salt thereof,
where R¹ is optionally substituted pyrazol-1-yl, optionally substituted pyrazol-3-yl, or optionally substituted pyrazol-5-yl;
R³ is morpholin-1-yl or piperidin-1-yl;
R^{A} is methyl or ethyl; and
R² is

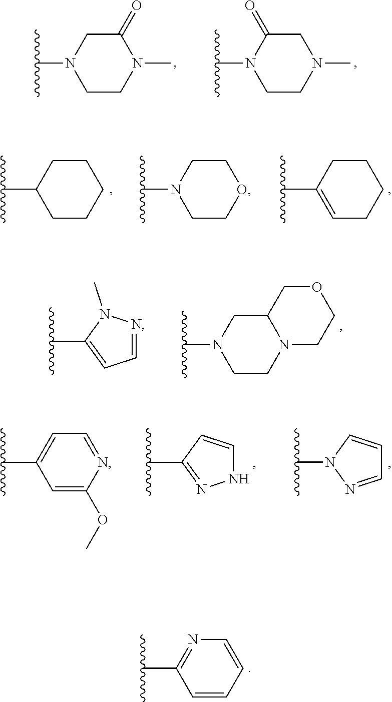

PIKfyve inhibitors described herein also include compounds of formula 30:

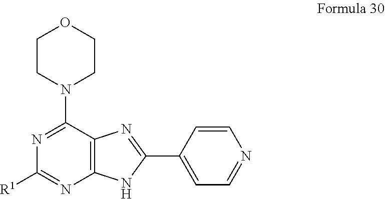

or a pharmaceutically acceptable salt thereof,
where R¹ is pyrazolyl monosubstituted with optionally substituted C₂-C₉ heterocyclyl or C₆-C₁₀ aryl.

PIKfyve inhibitors described herein also include compounds of formula 31:

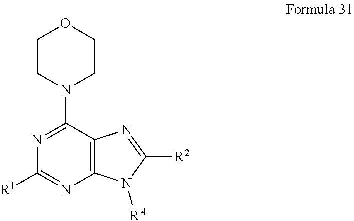

or a pharmaceutically acceptable salt thereof,
where R¹ is optionally substituted pyrazol-1-yl or pyrimidin-4-yl optionally substituted with optionally substituted C₁-C₆ alkyl;
R^{A} is methyl or difluoromethyl;
R² is pyridin-4-yl or

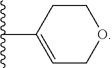 [SMILES: *C1=CCOCC1]

PIKfyve inhibitors described herein also include compounds of formula 32:

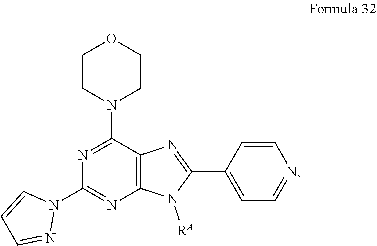

or a pharmaceutically acceptable salt thereof,
where R^{A} is or

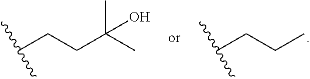

PIKfyve inhibitors described herein also include compounds of formula 33:

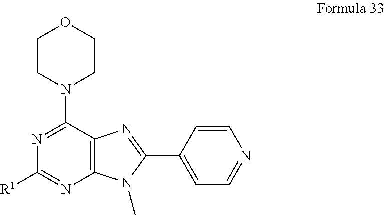

or a pharmaceutically acceptable salt thereof,
where R¹ is

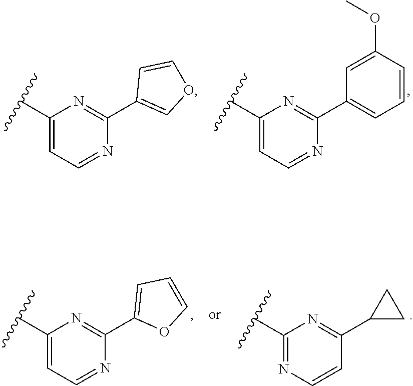

PIKfyve inhibitors described herein also include compounds of formula 34:

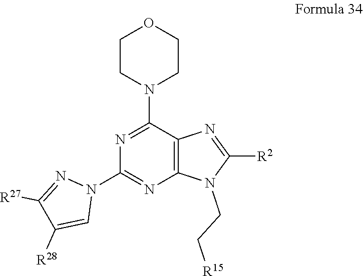

or a pharmaceutically acceptable salt thereof,
where R²⁷ is hydrogen, tetrahydropyran-3-yl, or tetrahydropyran-4-yl;
R²⁸ is hydrogen, methoxy, phenyl, methyl, difluoromethyl, optionally substituted cyclobutyl,
R¹⁵ is hydrogen or methoxy; and
R² is pyridin-4-yl or —O-pyridin-4-yl.

PIKfyve inhibitors described herein also include compounds of formula 35:

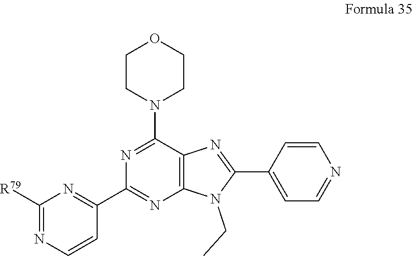

or a pharmaceutically acceptable salt thereof,
where R⁷⁹ is optionally substituted C₂-C₉ heterocyclyl or optionally substituted C₆-C₁₀ aryl.

PIKfyve inhibitors described herein also include compounds of formula 36:

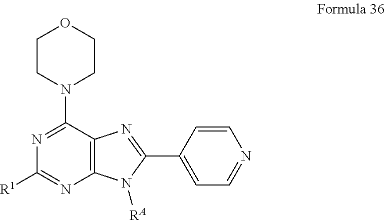

or a pharmaceutically acceptable salt thereof,
where R¹ is optionally substituted 4,5-dihydro-pyrazol-1-yl, optionally substituted benzopiperidin-7-yl, optionally substituted 1,2,3,4-tetrahydroquinolin-7-yl, optionally substituted imidazol-2-yl, optionally substituted piperidin-1-yl, or optionally substituted 1,2,4-triazol-3-yl, optionally substituted pyrazol-4-yl, optionally substituted 1,3,4-oxadiazol-2-yl, or optionally substituted pyridin-3-yl; and
R^{A} is methyl or ethyl.

PIKfyve inhibitors described herein also include compounds of formula 37:

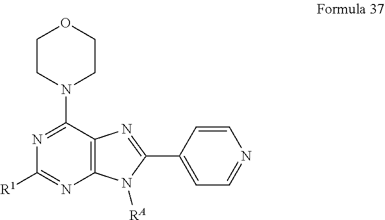

or a pharmaceutically acceptable salt thereof,
where R¹ is pyrazol-5-yl optionally substituted with C₂-C₉ heteroaryl, C₆-C₁₀ aryl, C₃-C₈ cycloalkyl or C₃-C₈ cycloalkyl C₁-C₆ alkyl; and
R^{A} is methyl or ethyl.

PIKfyve inhibitors described herein also include compounds of formula 38:

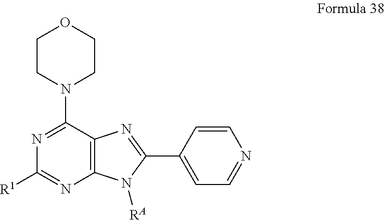

or a pharmaceutically acceptable salt thereof,
where R¹ is pyrazol-3-yl substituted with optionally substituted C₂-C₉ heteroaryl, optionally substituted C₃-C₈ cycloalkyl, optionally substituted C₂ alkyl, or optionally substituted C₆-C₁₀ aryl C₁-C₆ alkyl; and
R^{A} is methyl or ethyl.

PIKfyve inhibitors described herein also include compounds of formula 39:

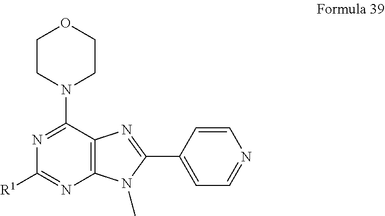

or a pharmaceutically acceptable salt thereof,
where R¹ is pyrazol-3-yl disubstituted with C₁-C₆ alkyl or C₆-C₁₀ aryl.

PIKfyve inhibitors described herein also include compounds of formula 40:

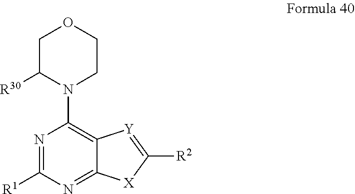

or a pharmaceutically acceptable salt thereof,
where Y is CH or N;
X is O, or S;
R¹ is optionally substituted morpholin-1-yl, optionally substituted pyrimidin-4-yl, —N(R^{1A})N═C(R^{1B})₂, optionally substituted pyrazol-3-yl, or optionally substituted indazol-4-yl;
R² is hydrogen or methyl; and
R³⁰ is optionally substituted pyridin-4-yl, optionally substituted pyrazol-3-yl, optionally substituted pyrazol-1-yl, or C₂-C₉ heterocycle C₁-C₆ alkyl substituted with —S(O)₂CH₃.

PIKfyve inhibitors described herein also include compounds of formula 41:

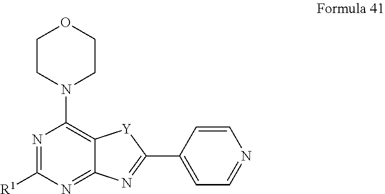

or a pharmaceutically acceptable salt thereof,
where Y is S or NR^{A}.
R¹ is optionally substituted pyrimidin-4-yl; and
R^{A} is optionally substituted C₁-C₆ alkyl.

PIKfyve inhibitors described herein also include compounds of formula 42:

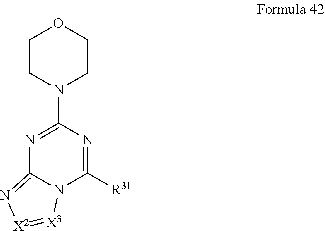

or a pharmaceutically acceptable salt thereof,
where X² and X³ are each, independently, N or CR³²
R³¹ is optionally substituted C₂-C₉ heteroaryl; and
R³² is optionally substituted C₂-C₉ heteroaryl.

PIKfyve inhibitors described herein also include compounds of formula 43:

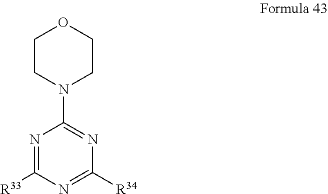

or a pharmaceutically acceptable salt thereof,
where R³³ is optionally substituted amino; and
R³⁴ is optionally substituted C₂-C₉ heteroaryl.

PIKfyve inhibitors described herein also include compounds of formula 44:

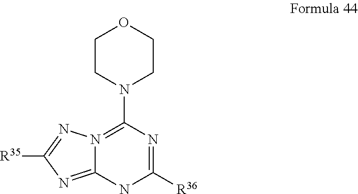

or a pharmaceutically acceptable salt thereof,
where R³⁵ and R³⁶ are each, independently, optionally substituted C₂-C₉ heteroaryl.

PIKfyve inhibitors described herein also include compounds of formula 45:

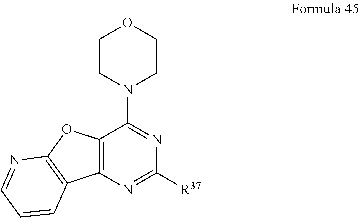

or a pharmaceutically acceptable salt thereof,
where R³⁷ is optionally substituted C₂-C₉ heteroaryl.

PIKfyve inhibitors described herein also include compounds of formula 46:

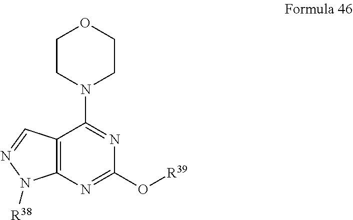

or a pharmaceutically acceptable salt thereof,
where R³⁸ is optionally substituted C₆-C₁₀ aryl; and
R³⁹ is optionally substituted C₂-C₉ heteroaryl C₁-C₆ alkyl.

PIKfyve inhibitors described herein also include compounds of formula 47:

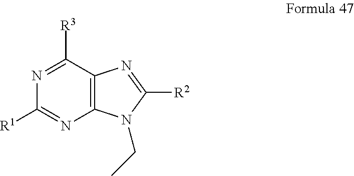

or a pharmaceutically acceptable salt thereof,
where R² is hydrogen, optionally substituted C₂-C₉ heteroaryl; optionally substituted C₂-C₉ heterocyclyl, or C₁-C₃ alkyl optionally substituted with hydroxyl, oxo, or dialkyl amino;
R¹ is optionally substituted pyrazol-1-yl, phenyl optionally substituted with optionally substituted C₂-C₉ heteroaryl or optionally substituted C₆-C₁₀ aryl, or —N(R^{1A})N═C(R^{1B})₂; and
R³ is

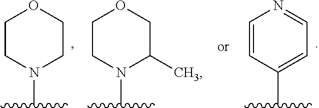

PIKfyve inhibitors described herein also include compounds of formula 48:

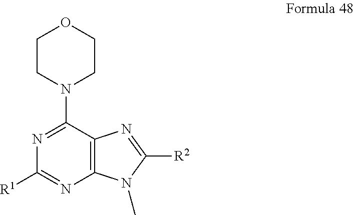

or a pharmaceutically acceptable salt thereof,
where R² is optionally substituted C₂-C₉ heteroaryl; and
R¹ is —N(R^{1A})N═C(R^{1B})₂.

PIKfyve inhibitors described herein also include compounds of formula 49:

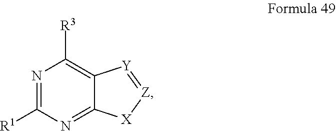

or a pharmaceutically acceptable salt thereof,
where
X is NR^{A}, S, or O;
Y is CR^{A} or N;
Z is CR² or N;
R¹ is hydrogen, optionally substituted C₁-C₆ alkyl, optionally substituted C₆-C₁₀ aryl, optionally substituted C₁-C₉ heterocyclyl, optionally substituted amino, optionally substituted C₃-C₈ cycloalkyl, optionally substituted C₂-C₉ heterocyclyl C₁-C₆ alkyl, optionally substituted C₂-C₉ heteroaryl C₁-C₆ alkyl, optionally substituted C₂-C₉ heteroaryl; —NHNHR^{1A}; —N(R^{1A})N═C(R^{1B})₂; —C(R^{1A})═N—N(R^{1B})₂; —C(R^{1A})═NOR^{1A}; or -Q¹-N(R^{1C})₂;
Q¹ is a bond, CH₂, or CO;
each R^{1A} is independently H, optionally substituted C₁-C₆ alkyl, optionally substituted C₆-C₁₀ aryl, or optionally substituted C₆-C₁₀ aryl C₁-C₆ alkyl;
one R^{1B} is independently H, optionally substituted C₁-C₆ alkyl, optionally substituted C₆-C₁₀ aryl, or optionally substituted C₂-C₉ heteroaryl; and the remaining R^{1B} is optionally substituted 01-C₆ alkyl, optionally substituted C₆-C₁₀ aryl, or optionally substituted C₂-C₉ heteroaryl;
each R^{1C} is independently H, optionally substituted C₁-C₆ alkyl, optionally substituted C₆-C₁₀ aryl, optionally substituted C₃-C₈ cycloalkyl, or optionally substituted C₂-C₉ heteroaryl; or both R^{1C}, together with the nitrogen atom to which they are attached, combine to form C₂-C₉ heterocyclyl or C₂-C₉ heteroaryl; R² is optionally substituted C₁-C₆ alkyl, optionally substituted C₆-C₁₀ aryl, optionally substituted C₂-C₉ heterocyclyl, optionally substituted C₂-C₉ heteroaryloxy, optionally substituted C₃-C₈ cycloalkyl, optionally substituted C₃-C₈ cycloalkenyl, or optionally substituted C₂-C₉ heteroaryl, -Q-N(R^{1c})₂; —S(O)r-R^{1A}; or —P(O)(R^{1A})₂; and each R^{A} is independently H, optionally substituted C₁-C₆ alkyl, optionally substituted C₆-C₁₀ aryl, optionally substituted C₂-C₉ heteroaryl, optionally substituted C₂-C₉ heteroaryl C₁-C₆ alkyl, optionally substituted C₃-C₈ cycloalkyl; or R² and R^{A}, together with the atoms to which they are attached, combine to form an optionally substituted C₃-C₄ heterocyclic ring, and the remaining R^{A}, if present, is H, optionally substituted C₁-C₆ alkyl, optionally substituted C₆-C₁₀ aryl, optionally substituted C₂-C₉ heteroaryl, optionally substituted C₂-C₉ heteroaryl C₁-C₆ alkyl, optionally substituted C₃-C₈ cycloalkyl;
r is 0, 1, or 2;
R³ is

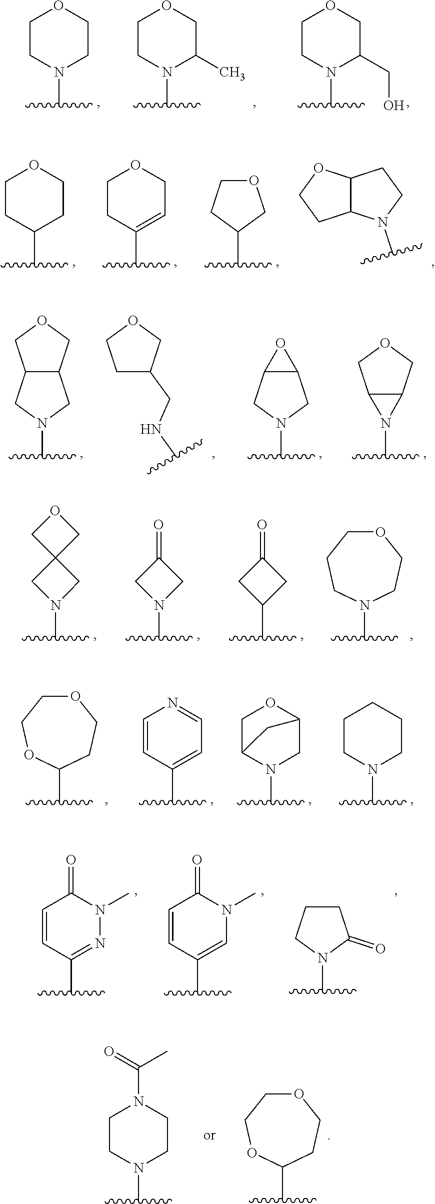

In some preferred embodiments, R¹ is optionally substituted C₂-C₉ heteroaryl including a 5-membered ring having a nitrogen atom at position 2 relative to the bond to the core, optionally substituted pyrimidin-6-yl, or optionally substituted benzodioxanyl. In some preferred embodiments, R² is optionally substituted C₆-C₁₀ aryl, optionally substituted C₁₋₉ heterocyclyl, or optionally C₁₋₉ substituted heteroaryl. In some preferred embodiments, Z is CR².

Exemplary PIKfyve inhibitors described herein also include any one of the compounds in Table 1.

TABLE 1

| # | Structure |
|---|---|
| 1 | 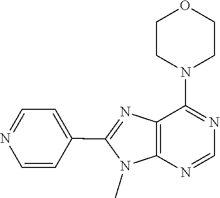 [SMILES: Cn1c(-c2ccncc2)nc2c(N3CCOCC3)ncnc21] |
| 2 | 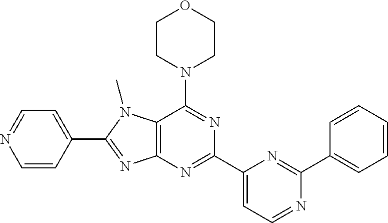 [SMILES: Cn1c(-c2ccncc2)nc2nc(-c3ccnc(-c4ccccc4)n3)nc(N3CCOCC3)c21] |
| 3 | 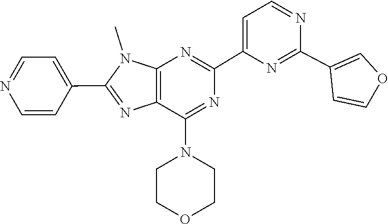 [SMILES: Cn1c(-c2ccncc2)nc2c(N3CCOCC3)nc(-c3ccnc(-c4ccoc4)n3)nc21] |
| 4 | 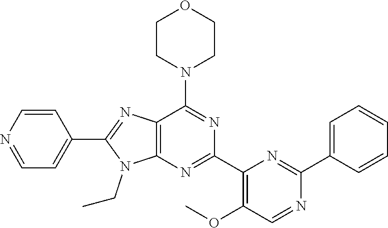 [SMILES: CCn1c(-c2ccncc2)nc2c(N3CCOCC3)nc(-c3nc(-c4ccccc4)ncc3OC)nc21] |
| 5 | 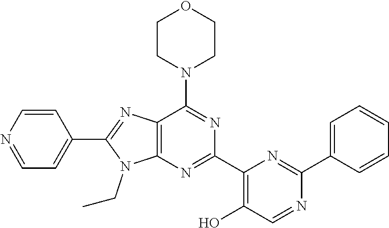 [SMILES: CCn1c(-c2ccncc2)nc2c(N3CCOCC3)nc(-c3nc(-c4ccccc4)ncc3O)nc21] |
| 6 | 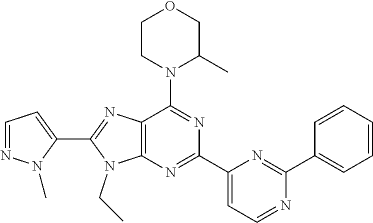 [SMILES: CCn1c(-c2ccnn2C)nc2c(N3CCOCC3C)nc(-c3ccnc(-c4ccccc4)n3)nc21] |
| 7 | 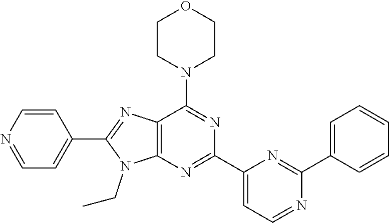 [SMILES: CCn1c(-c2ccncc2)nc2c(N3CCOCC3)nc(-c3ccnc(-c4ccccc4)n3)nc21] |
| 8 | 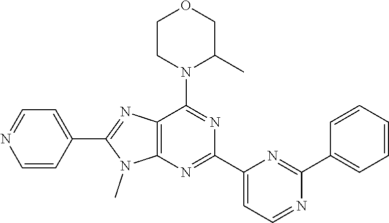 [SMILES: CC1COCCN1c1nc(-c2ccnc(-c3ccccc3)n2)nc2c1nc(-c1ccncc1)n2C] |
| 9 | 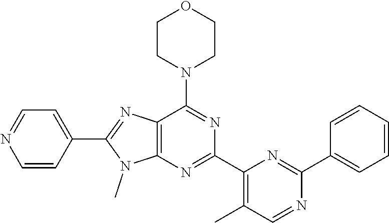 [SMILES: Cc1cnc(-c2ccccc2)nc1-c1nc(N2CCOCC2)c2nc(-c3ccncc3)n(C)c2n1] |
| 10 | 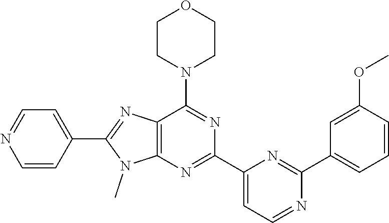 [SMILES: COc1cccc(-c2nccc(-c3nc(N4CCOCC4)c4nc(-c5ccncc5)n(C)c4n3)n2)c1] |
| 11 | 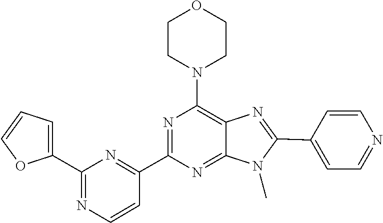 [SMILES: Cn1c(-c2ccncc2)nc2c(N3CCOCC3)nc(-c3ccnc(-c4ccco4)n3)nc21] |
| 12 | 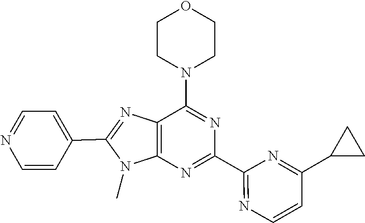 [SMILES: Cn1c(-c2ccncc2)nc2c(N3CCOCC3)nc(-c3nccc(C4CC4)n3)nc21] |
| 13 | 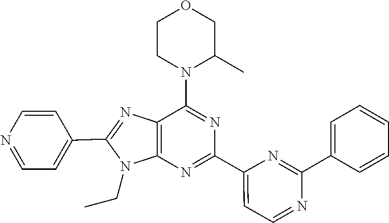 [SMILES: CCn1c(-c2ccncc2)nc2c(N3CCOCC3C)nc(-c3ccnc(-c4ccccc4)n3)nc21] |
| 14 | 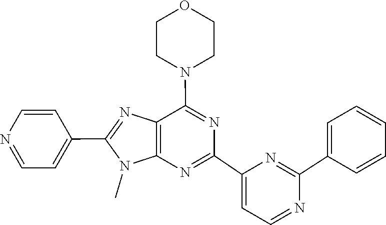 [SMILES: Cn1c(-c2ccncc2)nc2c(N3CCOCC3)nc(-c3ccnc(-c4ccccc4)n3)nc21] |
| 15 | 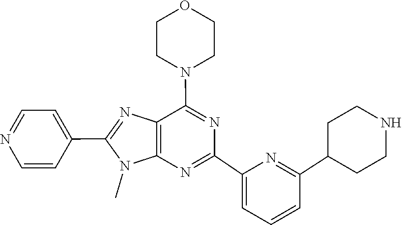 [SMILES: Cn1c(-c2ccncc2)nc2c(N3CCOCC3)nc(-c3cccc(C4CCNCC4)n3)nc21] |
| 16 | 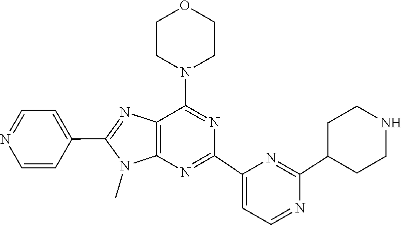 [SMILES: Cn1c(-c2ccncc2)nc2c(N3CCOCC3)nc(-c3ccnc(C4CCNCC4)n3)nc21] |
| 17 | 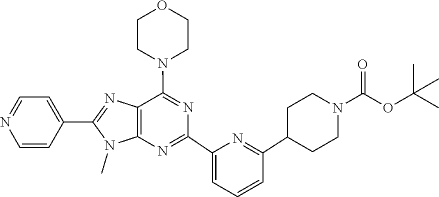 [SMILES: Cn1c(-c2ccncc2)nc2c(N3CCOCC3)nc(-c3cccc(C4CCN(C(=O)OC(C)(C)C)CC4)n3)nc21] |
| 18 | 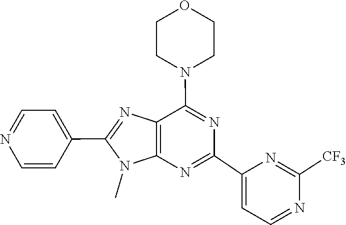 [SMILES: Cn1c(-c2ccncc2)nc2c(N3CCOCC3)nc(-c3ccnc(C(F)(F)F)n3)nc21] |
| 19 | 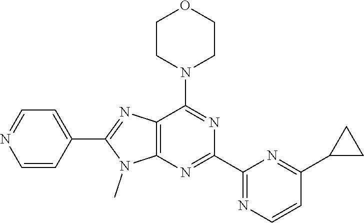 [SMILES: Cn1c(-c2ccncc2)nc2c(N3CCOCC3)nc(-c3nccc(C4CC4)n3)nc21] |
| 20 | 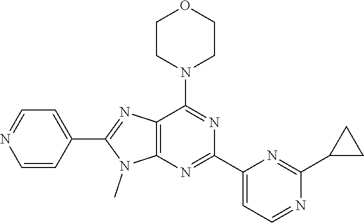 [SMILES: Cn1c(-c2ccncc2)nc2c(N3CCOCC3)nc(-c3ccnc(C4CC4)n3)nc21] |
| 21 | 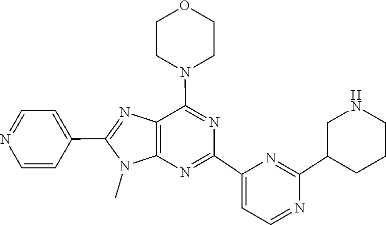 [SMILES: Cn1c(-c2ccncc2)nc2c(N3CCOCC3)nc(-c3ccnc(C4CCCNC4)n3)nc21] |
| 22 | 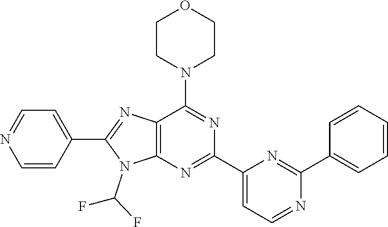 [SMILES: FC(F)n1c(-c2ccncc2)nc2c(N3CCOCC3)nc(-c3ccnc(-c4ccccc4)n3)nc21] |
| 24 | 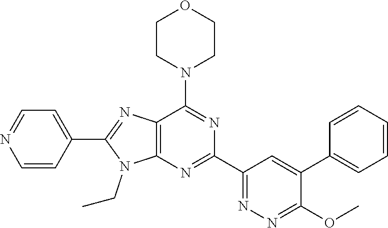 [SMILES: CCn1c(-c2ccncc2)nc2c(N3CCOCC3)nc(-c3cc(-c4ccccc4)c(OC)nn3)nc21] |
| 25 | 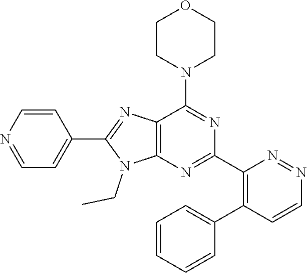 [SMILES: CCn1c(-c2ccncc2)nc2c(N3CCOCC3)nc(-c3nnccc3-c3ccccc3)nc21] |
| 26 | 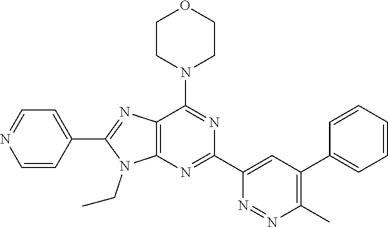 [SMILES: CCn1c(-c2ccncc2)nc2c(N3CCOCC3)nc(-c3cc(-c4ccccc4)c(C)nn3)nc21] |
| 27 | 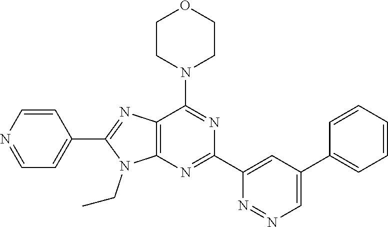 [SMILES: CCn1c(-c2ccncc2)nc2c(N3CCOCC3)nc(-c3cc(-c4ccccc4)cnn3)nc21] |
| 28 | 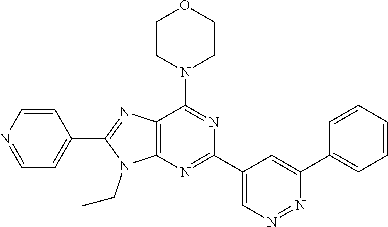 [SMILES: CCn1c(-c2ccncc2)nc2c(N3CCOCC3)nc(-c3cnnc(-c4ccccc4)c3)nc21] |
| 29 | 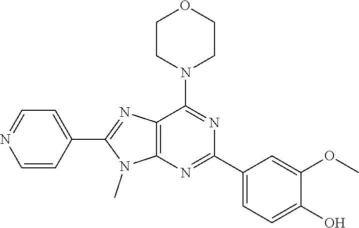 [SMILES: COc1cc(-c2nc(N3CCOCC3)c3nc(-c4ccncc4)n(C)c3n2)ccc1O] |
| 30 | 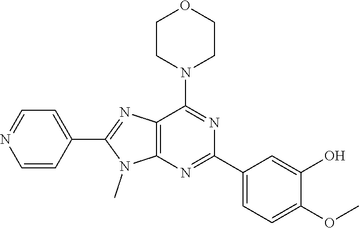 [SMILES: COc1ccc(-c2nc(N3CCOCC3)c3nc(-c4ccncc4)n(C)c3n2)cc1O] |
| 31 | 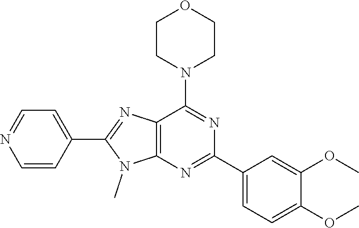 [SMILES: COc1ccc(-c2nc(N3CCOCC3)c3nc(-c4ccncc4)n(C)c3n2)cc1OC] |
| 32 | 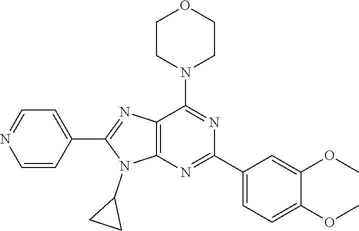 [SMILES: COc1ccc(-c2nc(N3CCOCC3)c3nc(-c4ccncc4)n(C4CC4)c3n2)cc1OC] |
| 33 | 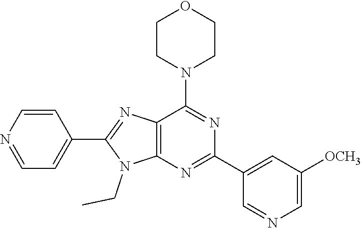 [SMILES: CCn1c(-c2ccncc2)nc2c(N3CCOCC3)nc(-c3cncc(OC)c3)nc21] |
| 34 | 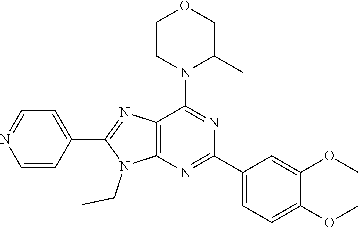 [SMILES: CCn1c(-c2ccncc2)nc2c(N3CCOCC3C)nc(-c3ccc(OC)c(OC)c3)nc21] |
| 35 | 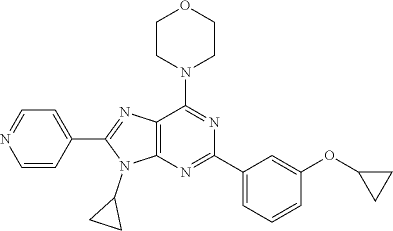 [SMILES: c1cc(OC2CC2)cc(-c2nc(N3CCOCC3)c3nc(-c4ccncc4)n(C4CC4)c3n2)c1] |
| 36 | 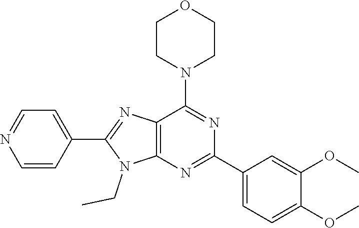 [SMILES: CCn1c(-c2ccncc2)nc2c(N3CCOCC3)nc(-c3ccc(OC)c(OC)c3)nc21] |
| 37 | 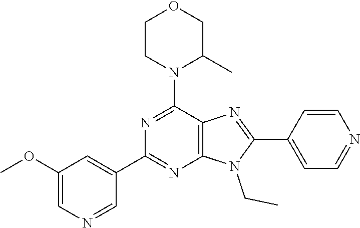 [SMILES: CCn1c(-c2ccncc2)nc2c(N3CCOCC3C)nc(-c3cncc(OC)c3)nc21] |
| 38 | 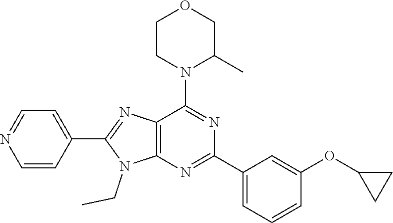 [SMILES: CCn1c(-c2ccncc2)nc2c(N3CCOCC3C)nc(-c3cccc(OC4CC4)c3)nc21] |
| 39 | 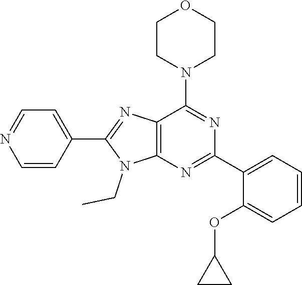 [SMILES: CCn1c(-c2ccncc2)nc2c(N3CCOCC3)nc(-c3ccccc3OC3CC3)nc21] |
| 40 | 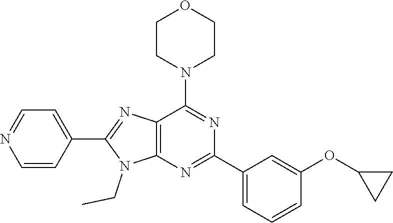 [SMILES: CCn1c(-c2ccncc2)nc2c(N3CCOCC3)nc(-c3cccc(OC4CC4)c3)nc21] |
| 41 | 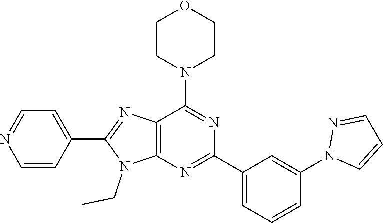 [SMILES: CCn1c(-c2ccncc2)nc2c(N3CCOCC3)nc(-c3cccc(-n4cccn4)c3)nc21] |
| 42 | 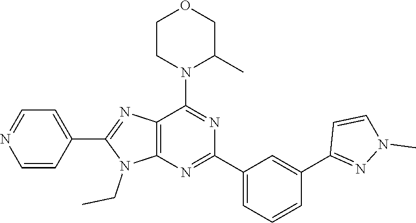 [SMILES: CCn1c(-c2ccncc2)nc2c(N3CCOCC3C)nc(-c3cccc(-c4ccn(C)n4)c3)nc21] |
| 43 | 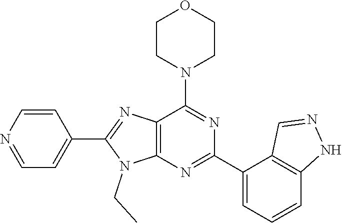 [SMILES: CCn1c(-c2ccncc2)nc2c(N3CCOCC3)nc(-c3cccc4[nH]ncc34)nc21] |
| 44 | 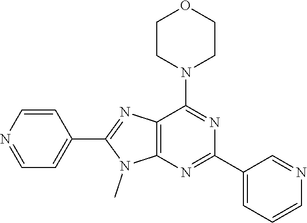 [SMILES: Cn1c(-c2ccncc2)nc2c(N3CCOCC3)nc(-c3cccnc3)nc21] |
| 45 | 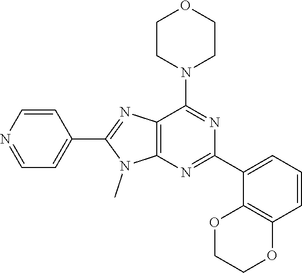 [SMILES: Cn1c(-c2ccncc2)nc2c(N3CCOCC3)nc(-c3cccc4c3OCCO4)nc21] |
| 46 | 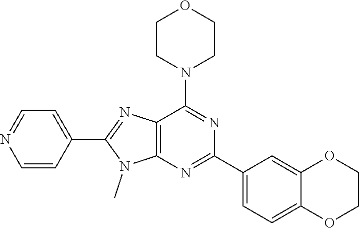 [SMILES: Cn1c(-c2ccncc2)nc2c(N3CCOCC3)nc(-c3ccc4c(c3)OCCO4)nc21] |
| 47 | 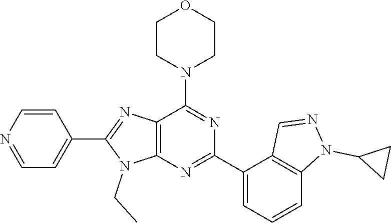 [SMILES: CCn1c(-c2ccncc2)nc2c(N3CCOCC3)nc(-c3cccc4c3cnn4C3CC3)nc21] |
| 48 | 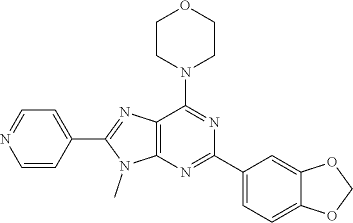 [SMILES: Cn1c(-c2ccncc2)nc2c(N3CCOCC3)nc(-c3ccc4c(c3)OCO4)nc21] |
| 49 | 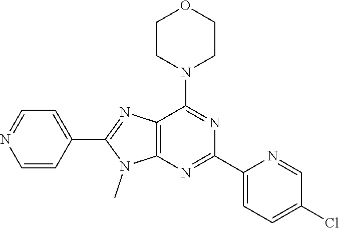 [SMILES: Cn1c(-c2ccncc2)nc2c(N3CCOCC3)nc(-c3ccc(Cl)cn3)nc21] |
| 50 | 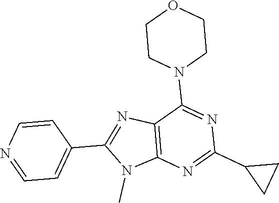 [SMILES: Cn1c(-c2ccncc2)nc2c(N3CCOCC3)nc(C3CC3)nc21] |
| 51 | 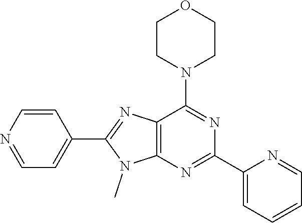 [SMILES: Cn1c(-c2ccncc2)nc2c(N3CCOCC3)nc(-c3ccccn3)nc21] |
| 52 | 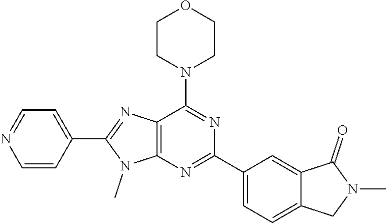 [SMILES: CN1Cc2ccc(-c3nc(N4CCOCC4)c4nc(-c5ccncc5)n(C)c4n3)cc2C1=O] |
| 53 | 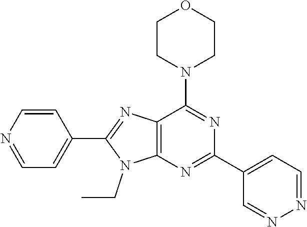 [SMILES: CCn1c(-c2ccncc2)nc2c(N3CCOCC3)nc(-c3ccnnc3)nc21] |
| 54 | 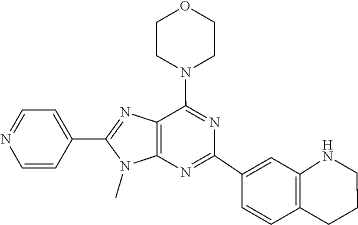 [SMILES: Cn1c(-c2ccncc2)nc2c(N3CCOCC3)nc(-c3ccc4c(c3)NCCC4)nc21] |
| 55 | 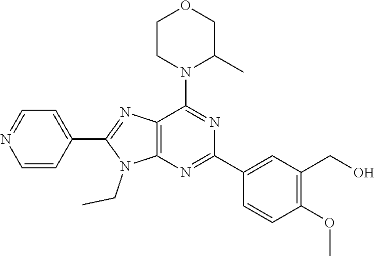 [SMILES: CCn1c(-c2ccncc2)nc2c(N3CCOCC3C)nc(-c3ccc(OC)c(CO)c3)nc21] |
| 56 | 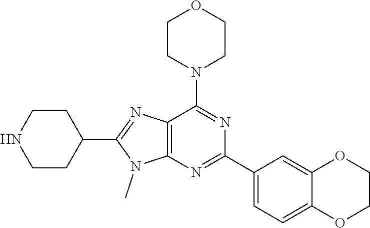 [SMILES: Cn1c(C2CCNCC2)nc2c(N3CCOCC3)nc(-c3ccc4c(c3)OCCO4)nc21] |
| 57 | 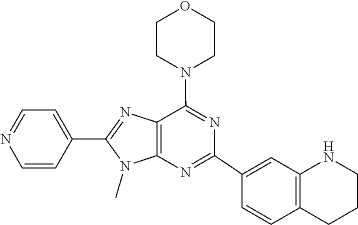 [SMILES: Cn1c(-c2ccncc2)nc2c(N3CCOCC3)nc(-c3ccc4c(c3)NCCC4)nc21] |
| 58 | 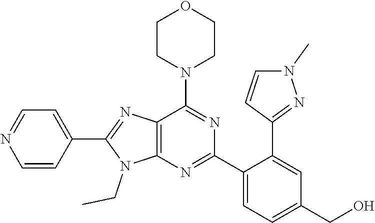 [SMILES: CCn1c(-c2ccncc2)nc2c(N3CCOCC3)nc(-c3ccc(CO)cc3-c3ccn(C)n3)nc21] |
| 59 | 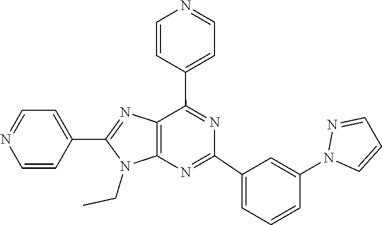 [SMILES: CCn1c(-c2ccncc2)nc2c(-c3ccncc3)nc(-c3cccc(-n4cccn4)c3)nc21] |
| 60 | 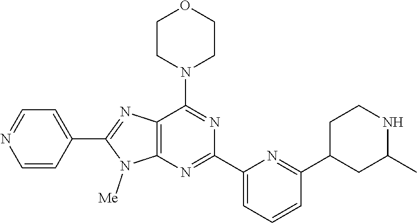 [SMILES: CC1CC(c2cccc(-c3nc(N4CCOCC4)c4nc(-c5ccncc5)n(C)c4n3)n2)CCN1] |
| 61 | 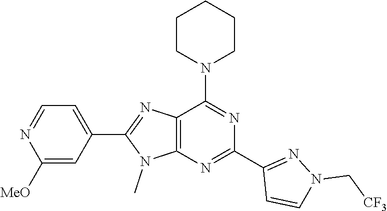 [SMILES: COc1cc(-c2nc3c(N4CCCCC4)nc(-c4ccn(CC(F)(F)F)n4)nc3n2C)ccn1] |
| 62 | 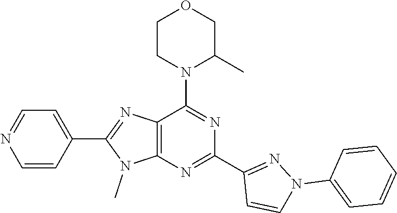 [SMILES: CC1COCCN1c1nc(-c2ccn(-c3ccccc3)n2)nc2c1nc(-c1ccncc1)n2C] |
| 63 | 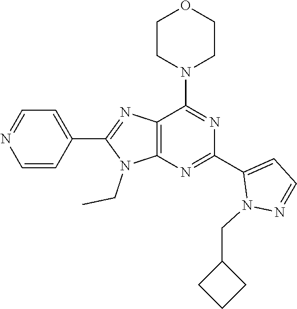 [SMILES: CCn1c(-c2ccncc2)nc2c(N3CCOCC3)nc(-c3ccnn3CC3CCC3)nc21] |
| 64 | 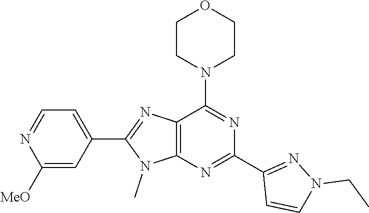 [SMILES: CCn1ccc(-c2nc(N3CCOCC3)c3nc(-c4ccnc(OC)c4)n(C)c3n2)n1] |
| 65 | 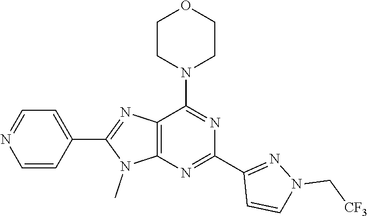 [SMILES: Cn1c(-c2ccncc2)nc2c(N3CCOCC3)nc(-c3ccn(CC(F)(F)F)n3)nc21] |
| 66 | 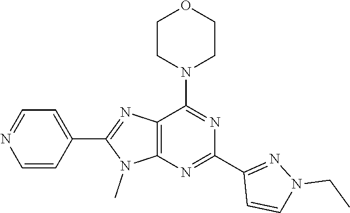 [SMILES: CCn1ccc(-c2nc(N3CCOCC3)c3nc(-c4ccncc4)n(C)c3n2)n1] |
| 67 | 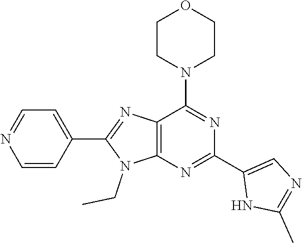 [SMILES: CCn1c(-c2ccncc2)nc2c(N3CCOCC3)nc(-c3cnc(C)[nH]3)nc21] |
| 68 | 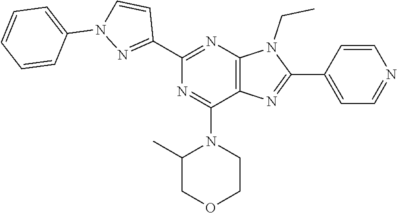 [SMILES: CCn1c(-c2ccncc2)nc2c(N3CCOCC3C)nc(-c3ccn(-c4ccccc4)n3)nc21] |
| 69 | 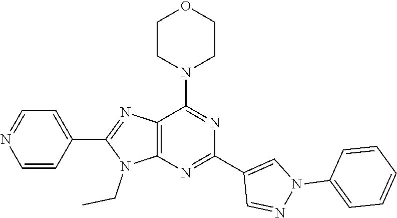 [SMILES: CCn1c(-c2ccncc2)nc2c(N3CCOCC3)nc(-c3cnn(-c4ccccc4)c3)nc21] |
| 70 | 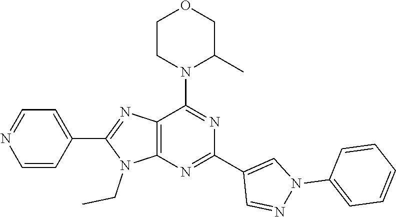 [SMILES: CCn1c(-c2ccncc2)nc2c(N3CCOCC3C)nc(-c3cnn(-c4ccccc4)c3)nc21] |
| 71 | 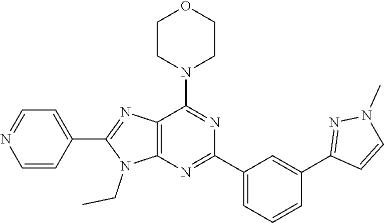 [SMILES: CCn1c(-c2ccncc2)nc2c(N3CCOCC3)nc(-c3cccc(-c4ccn(C)n4)c3)nc21] |
| 72 | 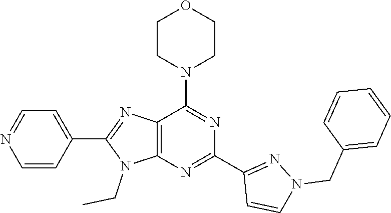 [SMILES: CCn1c(-c2ccncc2)nc2c(N3CCOCC3)nc(-c3ccn(Cc4ccccc4)n3)nc21] |
| 73 | 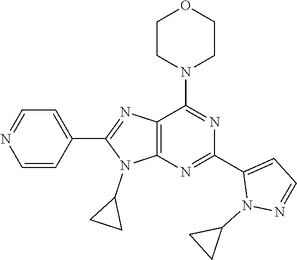 [SMILES: c1cc(-c2nc3c(N4CCOCC4)nc(-c4ccnn4C4CC4)nc3n2C2CC2)ccn1] |
| 74 | 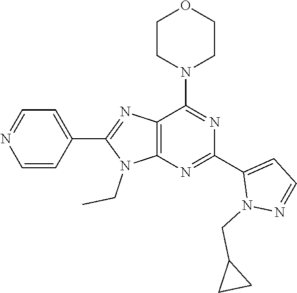 [SMILES: CCn1c(-c2ccncc2)nc2c(N3CCOCC3)nc(-c3ccnn3CC3CC3)nc21] |
| 75 | 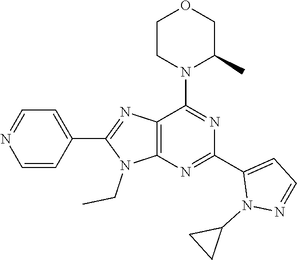 [SMILES: CCn1c(-c2ccncc2)nc2c(N3CCOC[C@H]3C)nc(-c3ccnn3C3CC3)nc21] |
| 76 | 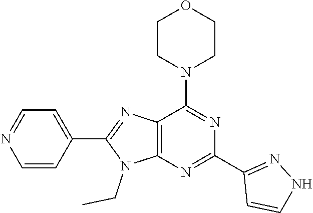 [SMILES: CCn1c(-c2ccncc2)nc2c(N3CCOCC3)nc(-c3cc[nH]n3)nc21] |
| 77 | 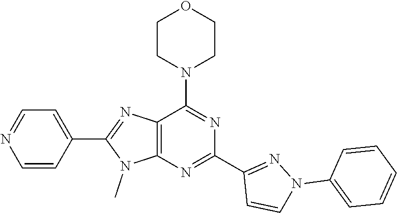 [SMILES: Cn1c(-c2ccncc2)nc2c(N3CCOCC3)nc(-c3ccn(-c4ccccc4)n3)nc21] |
| 78 | 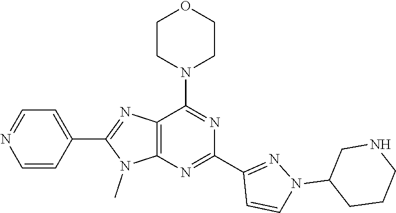 [SMILES: Cn1c(-c2ccncc2)nc2c(N3CCOCC3)nc(-c3ccn(C4CCCNC4)n3)nc21] |
| 79 | 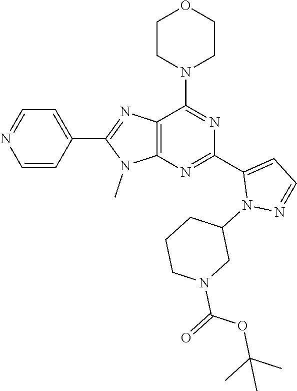 [SMILES: Cn1c(-c2ccncc2)nc2c(N3CCOCC3)nc(-c3ccnn3C3CCCN(C(=O)OC(C)(C)C)C3)nc21] |
| 80 | 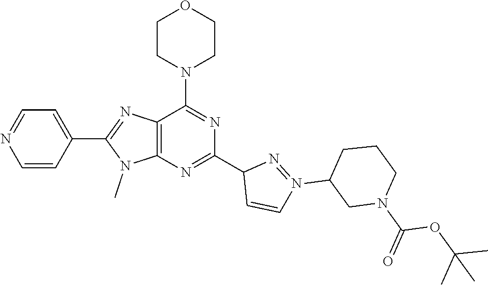 |
| 81 | 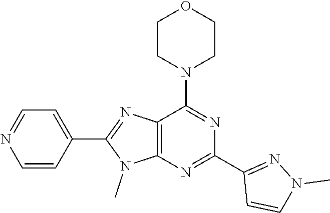 [SMILES: Cn1ccc(-c2nc(N3CCOCC3)c3nc(-c4ccncc4)n(C)c3n2)n1] |
| 82 | 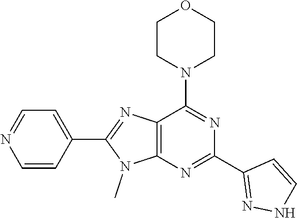 [SMILES: Cn1c(-c2ccncc2)nc2c(N3CCOCC3)nc(-c3cc[nH]n3)nc21] |
| 83 | 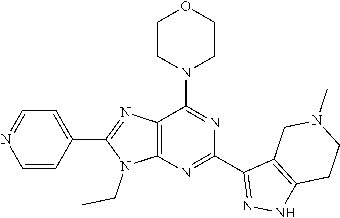 [SMILES: CCn1c(-c2ccncc2)nc2c(N3CCOCC3)nc(-c3n[nH]c4c3CN(C)CC4)nc21] |
| 84 | 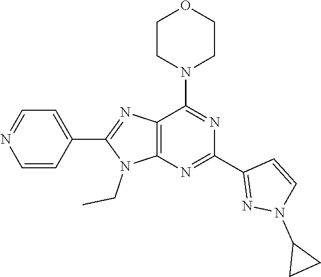 [SMILES: CCn1c(-c2ccncc2)nc2c(N3CCOCC3)nc(-c3ccn(C4CC4)n3)nc21] |
| 85 | 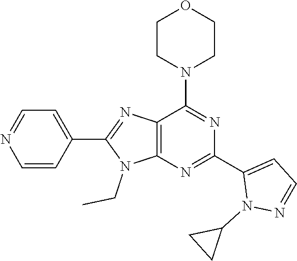 [SMILES: CCn1c(-c2ccncc2)nc2c(N3CCOCC3)nc(-c3ccnn3C3CC3)nc21] |
| 86 | 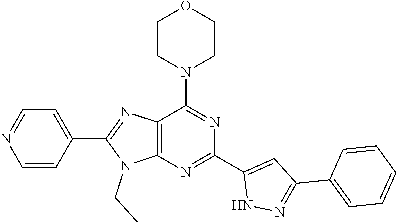 [SMILES: CCn1c(-c2ccncc2)nc2c(N3CCOCC3)nc(-c3cc(-c4ccccc4)n[nH]3)nc21] |
| 87 | 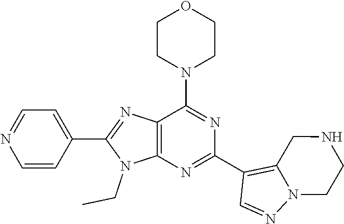 [SMILES: CCn1c(-c2ccncc2)nc2c(N3CCOCC3)nc(-c3cnn4c3CNCC4)nc21] |
| 88 | 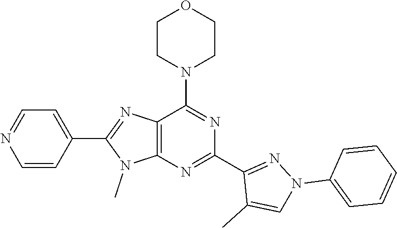 [SMILES: Cc1cn(-c2ccccc2)nc1-c1nc(N2CCOCC2)c2nc(-c3ccncc3)n(C)c2n1] |
| 89 | 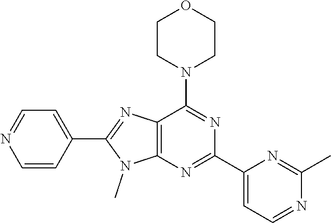 [SMILES: Cc1nccc(-c2nc(N3CCOCC3)c3nc(-c4ccncc4)n(C)c3n2)n1] |
| 90 | 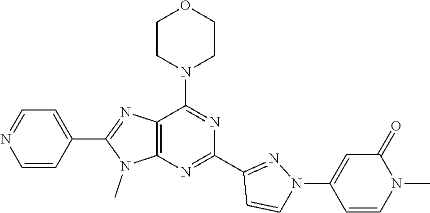 [SMILES: Cn1ccc(-n2ccc(-c3nc(N4CCOCC4)c4nc(-c5ccncc5)n(C)c4n3)n2)cc1=O] |
| 91 | 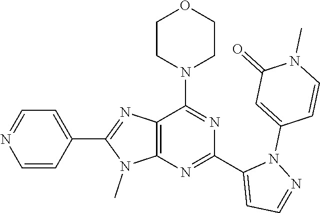 [SMILES: Cn1ccc(-n2nccc2-c2nc(N3CCOCC3)c3nc(-c4ccncc4)n(C)c3n2)cc1=O] |
| 92 | 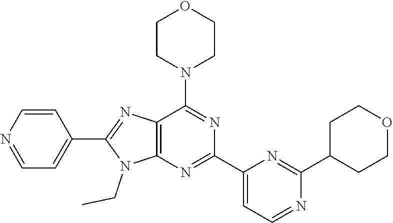 [SMILES: CCn1c(-c2ccncc2)nc2c(N3CCOCC3)nc(-c3ccnc(C4CCOCC4)n3)nc21] |
| 93 | 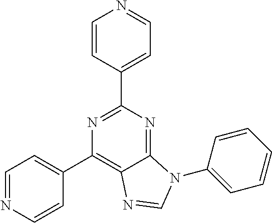 [SMILES: c1ccc(-n2cnc3c(-c4ccncc4)nc(-c4ccncc4)nc32)cc1] |
| 94 | 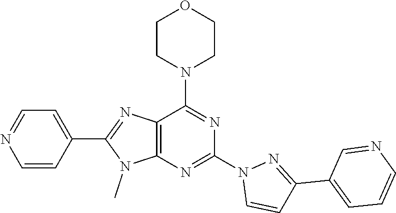 [SMILES: Cn1c(-c2ccncc2)nc2c(N3CCOCC3)nc(-n3ccc(-c4cccnc4)n3)nc21] |
| 95 | 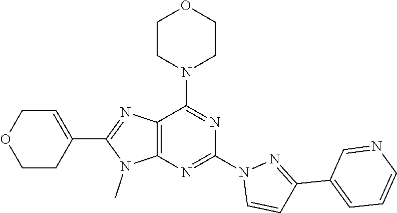 [SMILES: Cn1c(C2=CCOCC2)nc2c(N3CCOCC3)nc(-n3ccc(-c4cccnc4)n3)nc21] |
| 96 | 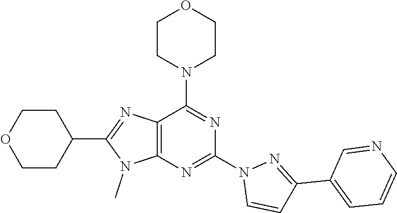 [SMILES: Cn1c(C2CCOCC2)nc2c(N3CCOCC3)nc(-n3ccc(-c4cccnc4)n3)nc21] |
| 97 | 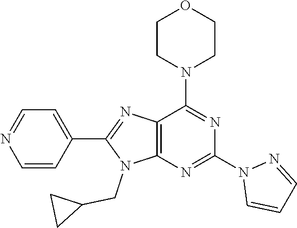 [SMILES: c1cnn(-c2nc(N3CCOCC3)c3nc(-c4ccncc4)n(CC4CC4)c3n2)c1] |
| 98 | 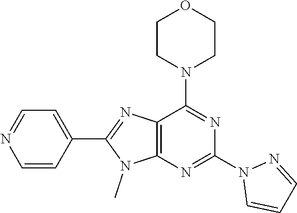 [SMILES: Cn1c(-c2ccncc2)nc2c(N3CCOCC3)nc(-n3cccn3)nc21] |
| 99 | 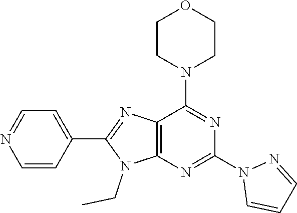 [SMILES: CCn1c(-c2ccncc2)nc2c(N3CCOCC3)nc(-n3cccn3)nc21] |
| 100 | 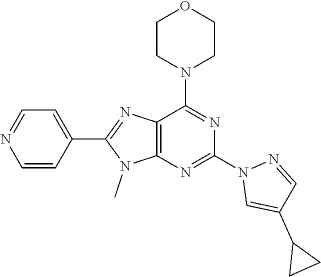 [SMILES: Cn1c(-c2ccncc2)nc2c(N3CCOCC3)nc(-n3cc(C4CC4)cn3)nc21] |
| 101 | 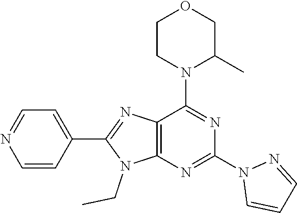 [SMILES: CCn1c(-c2ccncc2)nc2c(N3CCOCC3C)nc(-n3cccn3)nc21] |
| 102 | 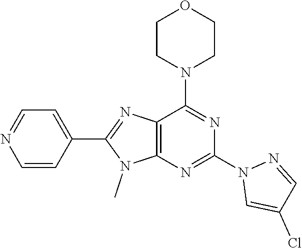 [SMILES: Cn1c(-c2ccncc2)nc2c(N3CCOCC3)nc(-n3cc(Cl)cn3)nc21] |
| 103 | 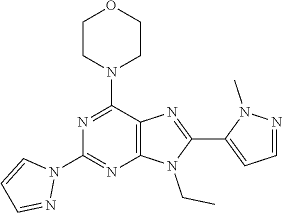 [SMILES: CCn1c(-c2ccnn2C)nc2c(N3CCOCC3)nc(-n3cccn3)nc21] |
| 104 | 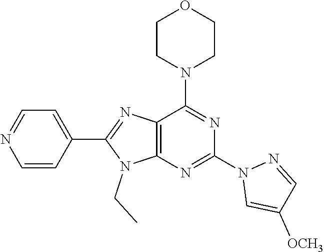 [SMILES: CCn1c(-c2ccncc2)nc2c(N3CCOCC3)nc(-n3cc(OC)cn3)nc21] |
| 105 | 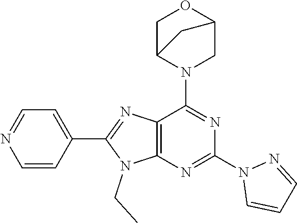 [SMILES: CCn1c(-c2ccncc2)nc2c(N3CC4CC3CO4)nc(-n3cccn3)nc21] |
| 106 | 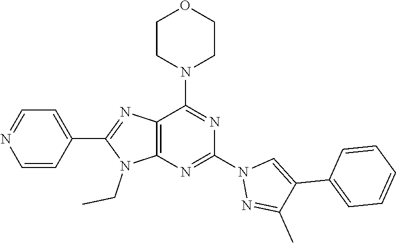 [SMILES: CCn1c(-c2ccncc2)nc2c(N3CCOCC3)nc(-n3cc(-c4ccccc4)c(C)n3)nc21] |
| 107 | 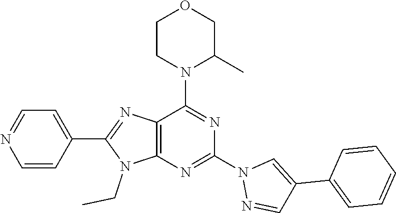 [SMILES: CCn1c(-c2ccncc2)nc2c(N3CCOCC3C)nc(-n3cc(-c4ccccc4)cn3)nc21] |
| 108 | 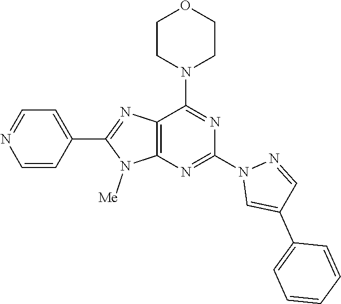 [SMILES: Cn1c(-c2ccncc2)nc2c(N3CCOCC3)nc(-n3cc(-c4ccccc4)cn3)nc21] |
| 109 | 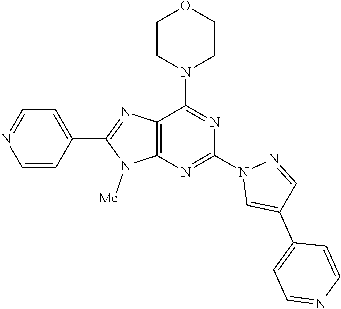 [SMILES: Cn1c(-c2ccncc2)nc2c(N3CCOCC3)nc(-n3cc(-c4ccncc4)cn3)nc21] |
| 110 | 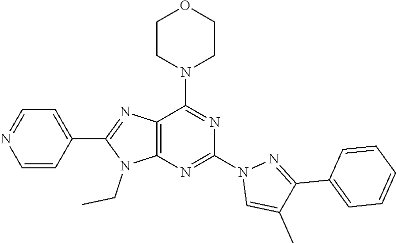 [SMILES: CCn1c(-c2ccncc2)nc2c(N3CCOCC3)nc(-n3cc(C)c(-c4ccccc4)n3)nc21] |
| 111 | 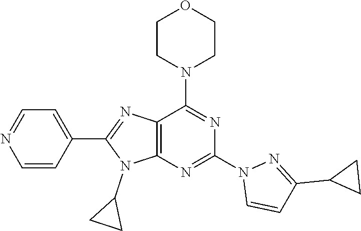 [SMILES: c1cc(-c2nc3c(N4CCOCC4)nc(-n4ccc(C5CC5)n4)nc3n2C2CC2)ccn1] |
| 112 | 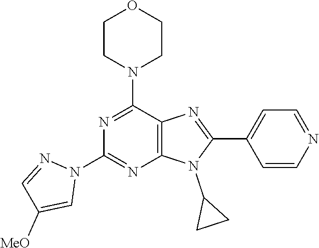 [SMILES: COc1cnn(-c2nc(N3CCOCC3)c3nc(-c4ccncc4)n(C4CC4)c3n2)c1] |
| 113 | 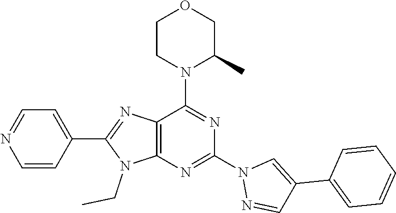 [SMILES: CCn1c(-c2ccncc2)nc2c(N3CCOC[C@H]3C)nc(-n3cc(-c4ccccc4)cn3)nc21] |
| 114 | 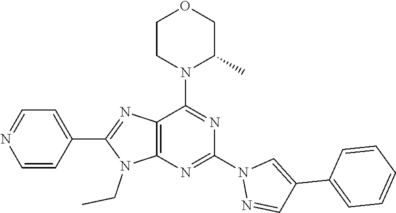 [SMILES: CCn1c(-c2ccncc2)nc2c(N3CCOC[C@@H]3C)nc(-n3cc(-c4ccccc4)cn3)nc21] |
| 115 | 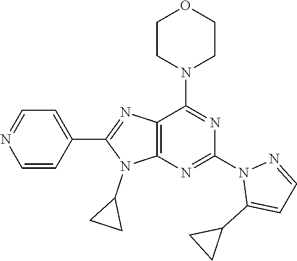 [SMILES: c1cc(-c2nc3c(N4CCOCC4)nc(-n4nccc4C4CC4)nc3n2C2CC2)ccn1] |
| 116 | 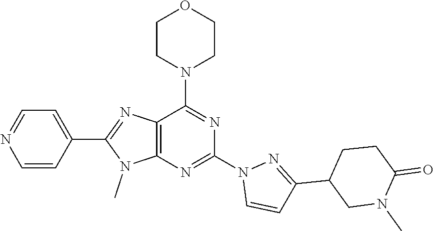 [SMILES: CN1CC(c2ccn(-c3nc(N4CCOCC4)c4nc(-c5ccncc5)n(C)c4n3)n2)CCC1=O] |
| 117 | 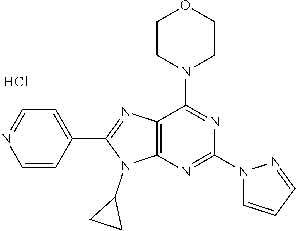 [SMILES: c1cnn(-c2nc(N3CCOCC3)c3nc(-c4ccncc4)n(C4CC4)c3n2)c1 ; Cl] |
| 118 | 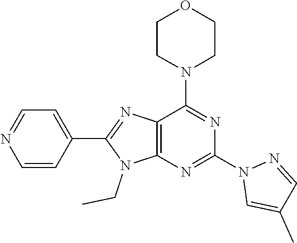 |
| 119 | 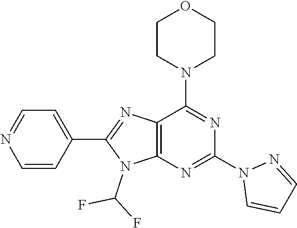 [SMILES: FC(F)n1c(-c2ccncc2)nc2c(N3CCOCC3)nc(-n3cccn3)nc21] |
| 120 | 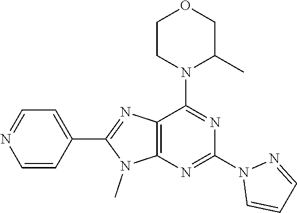 [SMILES: CC1COCCN1c1nc(-n2cccn2)nc2c1nc(-c1ccncc1)n2C] |
| 121 | 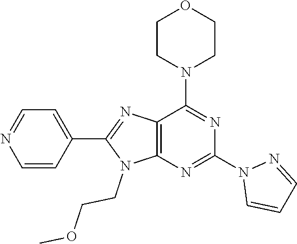 [SMILES: COCCn1c(-c2ccncc2)nc2c(N3CCOCC3)nc(-n3cccn3)nc21] |
| 122 | 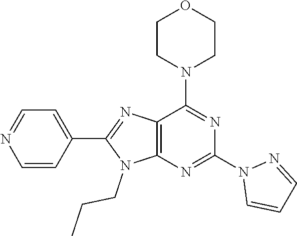 [SMILES: CCCn1c(-c2ccncc2)nc2c(N3CCOCC3)nc(-n3cccn3)nc21] |
| 123 | 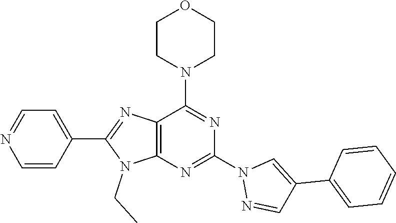 [SMILES: CCn1c(-c2ccncc2)nc2c(N3CCOCC3)nc(-n3cc(-c4ccccc4)cn3)nc21] |
| 124 | 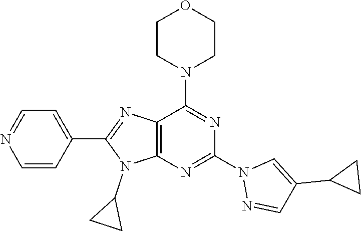 [SMILES: c1cc(-c2nc3c(N4CCOCC4)nc(-n4cc(C5CC5)cn4)nc3n2C2CC2)ccn1] |
| 125 | 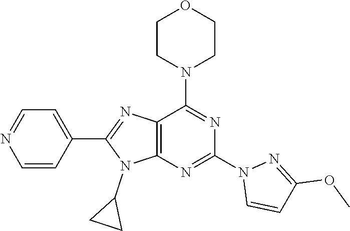 [SMILES: COc1ccn(-c2nc(N3CCOCC3)c3nc(-c4ccncc4)n(C4CC4)c3n2)n1] |
| 126 | 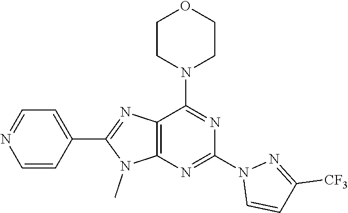 [SMILES: Cn1c(-c2ccncc2)nc2c(N3CCOCC3)nc(-n3ccc(C(F)(F)F)n3)nc21] |
| 127 | 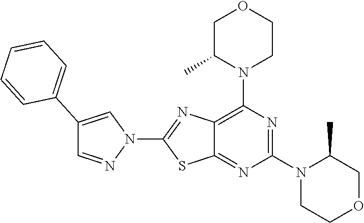 [SMILES: C[C@@H]1COCCN1c1nc(N2CCOC[C@@H]2C)nc2sc(-n3cc(-c4ccccc4)cn3)nc12] |
| 128 | 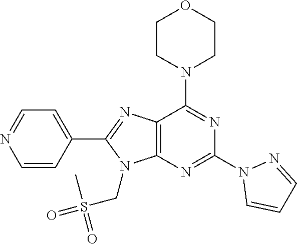 [SMILES: CS(=O)(=O)Cn1c(-c2ccncc2)nc2c(N3CCOCC3)nc(-n3cccn3)nc21] |
| 129 | 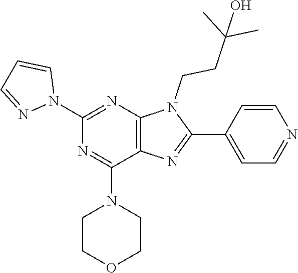 [SMILES: CC(C)(O)CCn1c(-c2ccncc2)nc2c(N3CCOCC3)nc(-n3cccn3)nc21] |
| 130 | 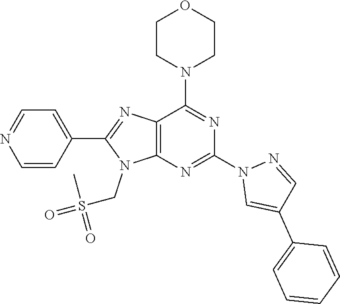 [SMILES: CS(=O)(=O)Cn1c(-c2ccncc2)nc2c(N3CCOCC3)nc(-n3cc(-c4ccccc4)cn3)nc21] |
| 131 | 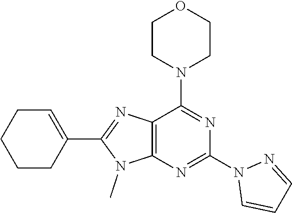 [SMILES: Cn1c(C2=CCCCC2)nc2c(N3CCOCC3)nc(-n3cccn3)nc21] |
| 132 | 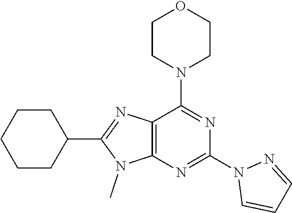 [SMILES: Cn1c(C2CCCCC2)nc2c(N3CCOCC3)nc(-n3cccn3)nc21] |
| 133 | 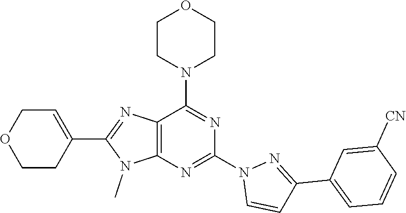 [SMILES: Cn1c(C2=CCOCC2)nc2c(N3CCOCC3)nc(-n3ccc(-c4cccc(C#N)c4)n3)nc21] |
| 134 | 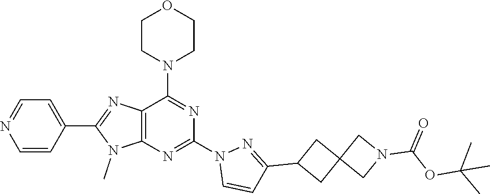 [SMILES: Cn1c(-c2ccncc2)nc2c(N3CCOCC3)nc(-n3ccc(C4CC5(C4)CN(C(=O)OC(C)(C)C)C5)n3)nc21] |
| 135 | 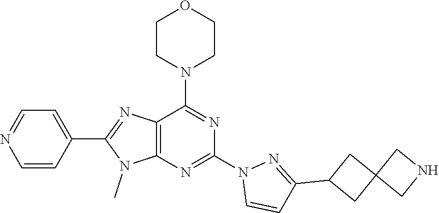 [SMILES: Cn1c(-c2ccncc2)nc2c(N3CCOCC3)nc(-n3ccc(C4CC5(CNC5)C4)n3)nc21] |
| 136 | 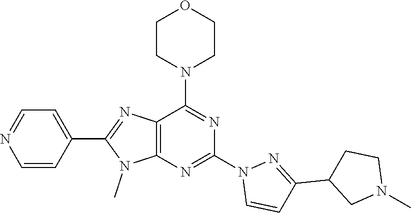 [SMILES: CN1CCC(c2ccn(-c3nc(N4CCOCC4)c4nc(-c5ccncc5)n(C)c4n3)n2)C1] |
| 137 | 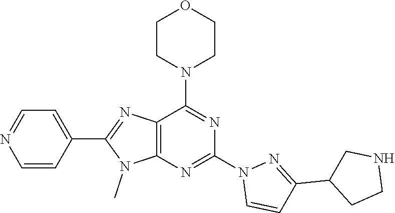 [SMILES: Cn1c(-c2ccncc2)nc2c(N3CCOCC3)nc(-n3ccc(C4CCNC4)n3)nc21] |
| 138 | 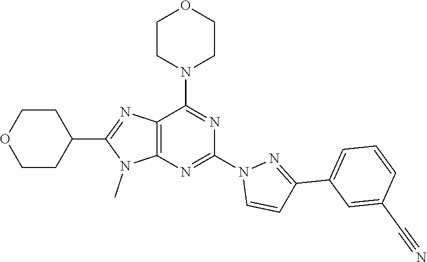 [SMILES: Cn1c(C2CCOCC2)nc2c(N3CCOCC3)nc(-n3ccc(-c4cccc(C#N)c4)n3)nc21] |
| 139 | 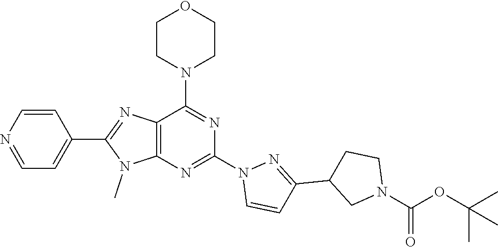 [SMILES: Cn1c(-c2ccncc2)nc2c(N3CCOCC3)nc(-n3ccc(C4CCN(C(=O)OC(C)(C)C)C4)n3)nc21] |
| 140 | 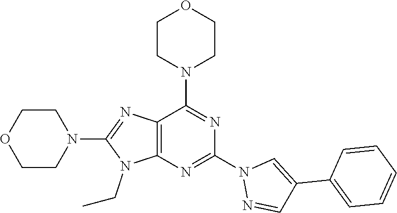 [SMILES: CCn1c(N2CCOCC2)nc2c(N3CCOCC3)nc(-n3cc(-c4ccccc4)cn3)nc21] |
| 141 | 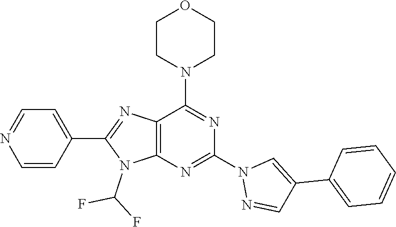 [SMILES: FC(F)n1c(-c2ccncc2)nc2c(N3CCOCC3)nc(-n3cc(-c4ccccc4)cn3)nc21] |
| 142 | 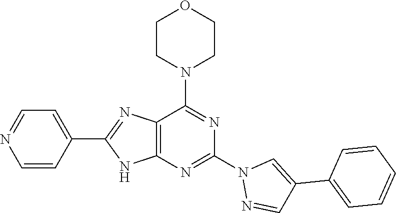 [SMILES: c1ccc(-c2cnn(-c3nc(N4CCOCC4)c4nc(-c5ccncc5)[nH]c4n3)c2)cc1] |
| 143 | 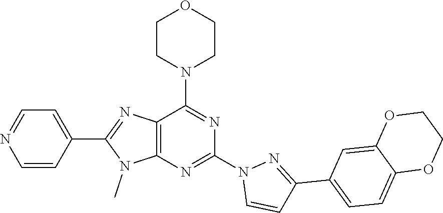 [SMILES: Cn1c(-c2ccncc2)nc2c(N3CCOCC3)nc(-n3ccc(-c4ccc5c(c4)OCCO5)n3)nc21] |
| 144 | 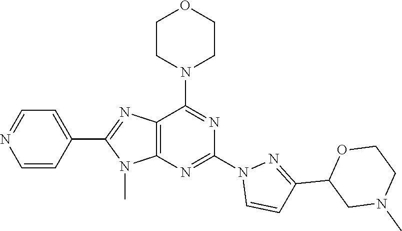 [SMILES: CN1CCOC(c2ccn(-c3nc(N4CCOCC4)c4nc(-c5ccncc5)n(C)c4n3)n2)C1] |
| 145 | 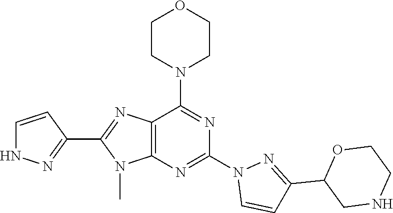 [SMILES: Cn1c(-c2cc[nH]n2)nc2c(N3CCOCC3)nc(-n3ccc(C4CNCCO4)n3)nc21] |
| 146 | 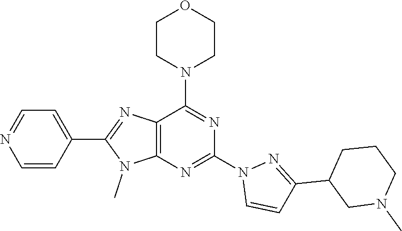 [SMILES: CN1CCCC(c2ccn(-c3nc(N4CCOCC4)c4nc(-c5ccncc5)n(C)c4n3)n2)C1] |
| 147 | 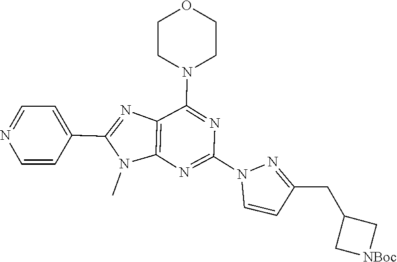 [SMILES: Cn1c(-c2ccncc2)nc2c(N3CCOCC3)nc(-n3ccc(CC4CN(C(=O)OC(C)(C)C)C4)n3)nc21] |
| 148 | 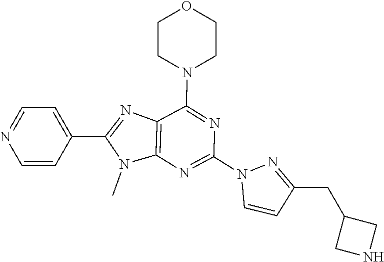 [SMILES: Cn1c(-c2ccncc2)nc2c(N3CCOCC3)nc(-n3ccc(CC4CNC4)n3)nc21] |
| 149 | 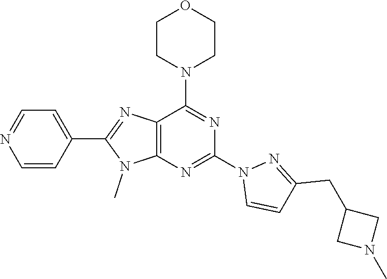 [SMILES: CN1CC(Cc2ccn(-c3nc(N4CCOCC4)c4nc(-c5ccncc5)n(C)c4n3)n2)C1] |
| 150 | 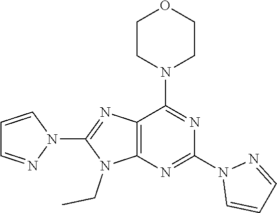 [SMILES: CCn1c(-n2cccn2)nc2c(N3CCOCC3)nc(-n3cccn3)nc21] |
| 151 | 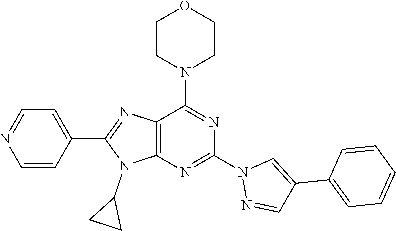 [SMILES: c1ccc(-c2cnn(-c3nc(N4CCOCC4)c4nc(-c5ccncc5)n(C5CC5)c4n3)c2)cc1] |
| 152 | 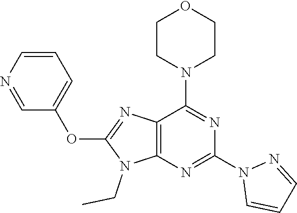 [SMILES: CCn1c(Oc2cccnc2)nc2c(N3CCOCC3)nc(-n3cccn3)nc21] |
| 153 | 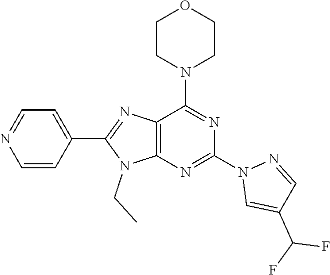 [SMILES: CCn1c(-c2ccncc2)nc2c(N3CCOCC3)nc(-n3cc(C(F)F)cn3)nc21] |
| 154 | 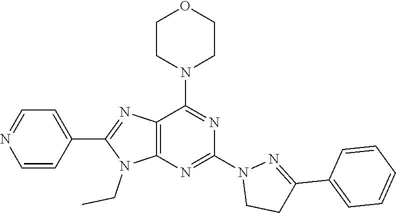 [SMILES: CCn1c(-c2ccncc2)nc2c(N3CCOCC3)nc(N3CCC(c4ccccc4)=N3)nc21] |
| 155 | 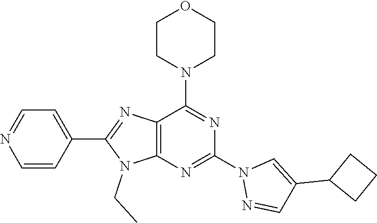 [SMILES: CCn1c(-c2ccncc2)nc2c(N3CCOCC3)nc(-n3cc(C4CCC4)cn3)nc21] |
| 156 | 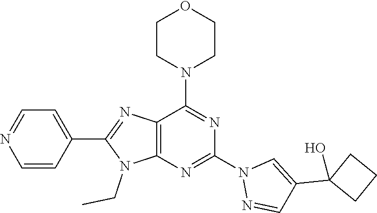 [SMILES: CCn1c(-c2ccncc2)nc2c(N3CCOCC3)nc(-n3cc(C4(O)CCC4)cn3)nc21] |
| 157 | 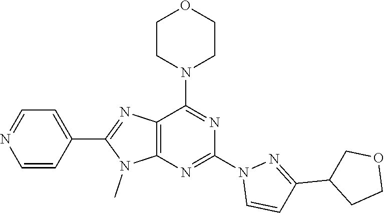 [SMILES: Cn1c(-c2ccncc2)nc2c(N3CCOCC3)nc(-n3ccc(C4CCOC4)n3)nc21] |
| 158 | 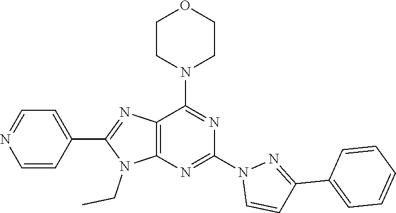 [SMILES: CCn1c(-c2ccncc2)nc2c(N3CCOCC3)nc(-n3ccc(-c4ccccc4)n3)nc21] |
| 159 | 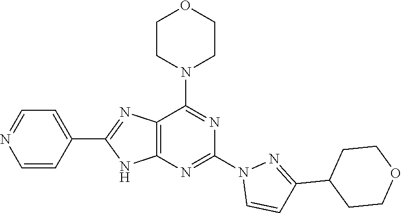 [SMILES: c1cc(-c2nc3c(N4CCOCC4)nc(-n4ccc(C5CCOCC5)n4)nc3[nH]2)ccn1] |
| 160 | 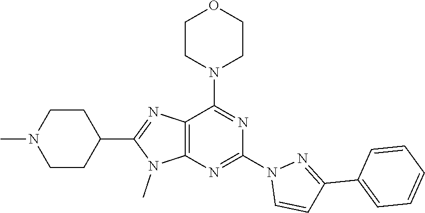 [SMILES: CN1CCC(c2nc3c(N4CCOCC4)nc(-n4ccc(-c5ccccc5)n4)nc3n2C)CC1] |
| 161 | 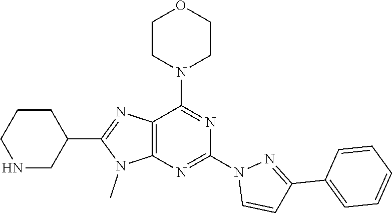 [SMILES: Cn1c(C2CCCNC2)nc2c(N3CCOCC3)nc(-n3ccc(-c4ccccc4)n3)nc21] |
| 162 | 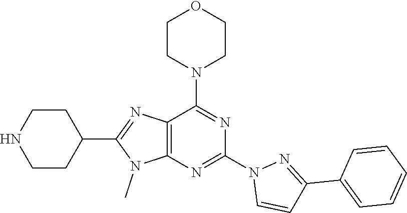 [SMILES: Cn1c(C2CCNCC2)nc2c(N3CCOCC3)nc(-n3ccc(-c4ccccc4)n3)nc21] |
| 163 | 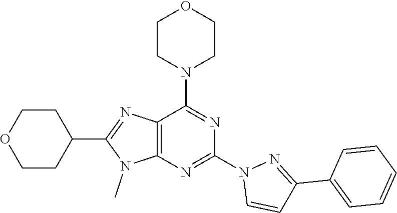 [SMILES: Cn1c(C2CCOCC2)nc2c(N3CCOCC3)nc(-n3ccc(-c4ccccc4)n3)nc21] |
| 165 | 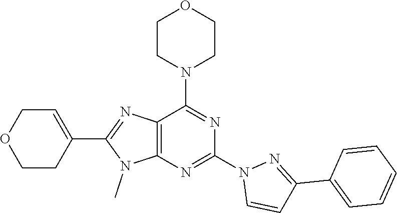 [SMILES: Cn1c(C2=CCOCC2)nc2c(N3CCOCC3)nc(-n3ccc(-c4ccccc4)n3)nc21] |
| 166 | 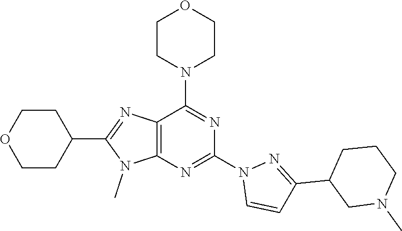 [SMILES: CN1CCCC(c2ccn(-c3nc(N4CCOCC4)c4nc(C5CCOCC5)n(C)c4n3)n2)C1] |
| 167 | 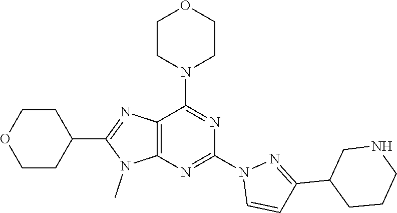 [SMILES: Cn1c(C2CCOCC2)nc2c(N3CCOCC3)nc(-n3ccc(C4CCCNC4)n3)nc21] |
| 168 | 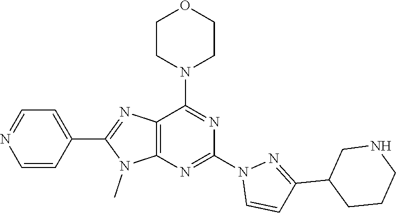 [SMILES: Cn1c(-c2ccncc2)nc2c(N3CCOCC3)nc(-n3ccc(C4CCCNC4)n3)nc21] |
| 169 | 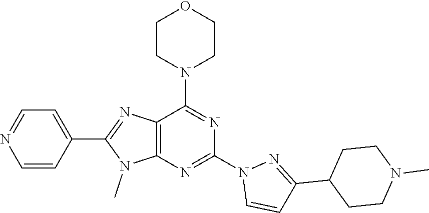 [SMILES: CN1CCC(c2ccn(-c3nc(N4CCOCC4)c4nc(-c5ccncc5)n(C)c4n3)n2)CC1] |
| 171 | 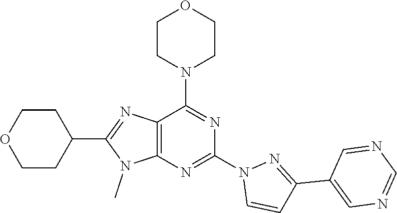 [SMILES: Cn1c(C2CCOCC2)nc2c(N3CCOCC3)nc(-n3ccc(-c4cncnc4)n3)nc21] |
| 172 | 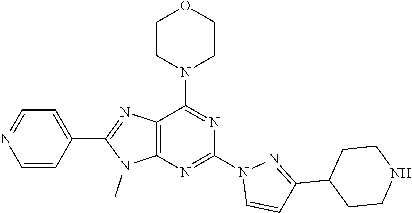 [SMILES: Cn1c(-c2ccncc2)nc2c(N3CCOCC3)nc(-n3ccc(C4CCNCC4)n3)nc21] |
| 173 | 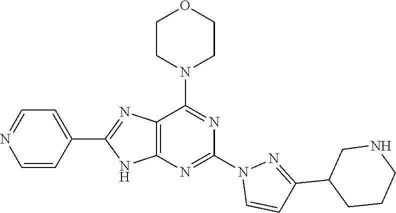 [SMILES: c1cc(-c2nc3c(N4CCOCC4)nc(-n4ccc(C5CCCNC5)n4)nc3[nH]2)ccn1] |
| 174 | 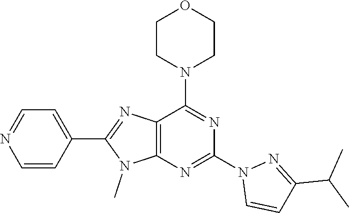 [SMILES: CC(C)c1ccn(-c2nc(N3CCOCC3)c3nc(-c4ccncc4)n(C)c3n2)n1] |
| 175 | 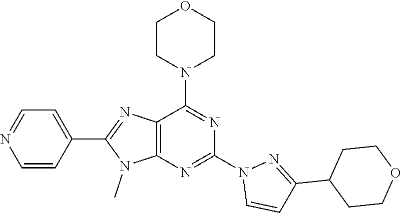 [SMILES: Cn1c(-c2ccncc2)nc2c(N3CCOCC3)nc(-n3ccc(C4CCOCC4)n3)nc21] |
| 176 | 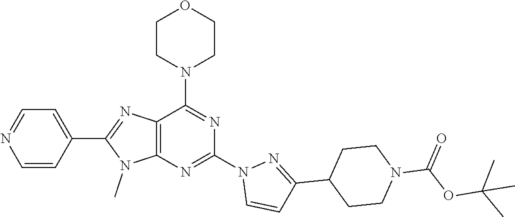 [SMILES: Cn1c(-c2ccncc2)nc2c(N3CCOCC3)nc(-n3ccc(C4CCN(C(=O)OC(C)(C)C)CC4)n3)nc21] |
| 177 | 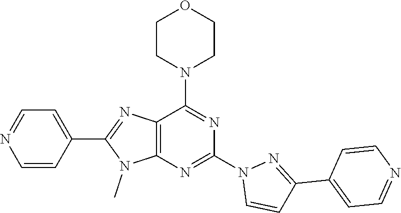 [SMILES: Cn1c(-c2ccncc2)nc2c(N3CCOCC3)nc(-n3ccc(-c4ccncc4)n3)nc21] |
| 178 | 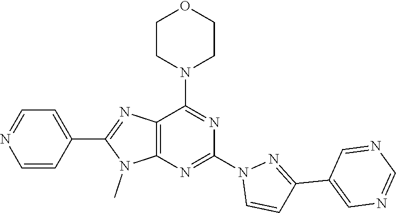 [SMILES: Cn1c(-c2ccncc2)nc2c(N3CCOCC3)nc(-n3ccc(-c4cncnc4)n3)nc21] |
| 179 | 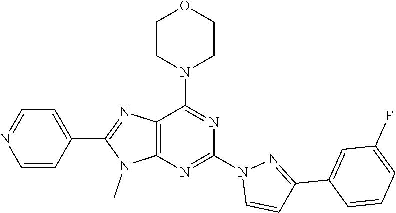 [SMILES: Cn1c(-c2ccncc2)nc2c(N3CCOCC3)nc(-n3ccc(-c4cccc(F)c4)n3)nc21] |
| 180 | 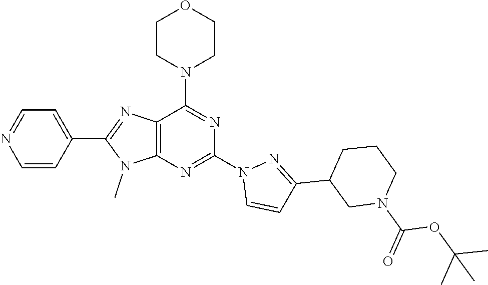 [SMILES: Cn1c(-c2ccncc2)nc2c(N3CCOCC3)nc(-n3ccc(C4CCCN(C(=O)OC(C)(C)C)C4)n3)nc21] |
| 181 | 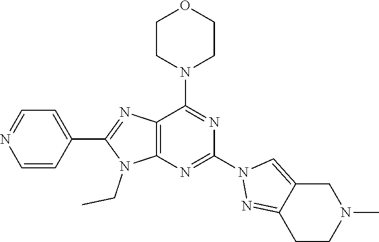 [SMILES: CCn1c(-c2ccncc2)nc2c(N3CCOCC3)nc(-n3cc4c(n3)CCN(C)C4)nc21] |
| 182 | 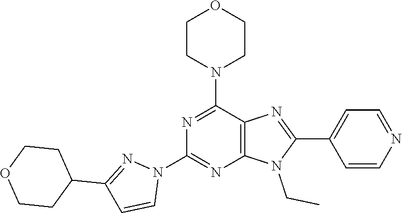 [SMILES: CCn1c(-c2ccncc2)nc2c(N3CCOCC3)nc(-n3ccc(C4CCOCC4)n3)nc21] |
| 183 | 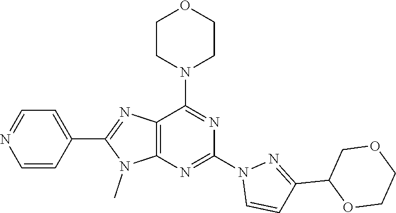 [SMILES: Cn1c(-c2ccncc2)nc2c(N3CCOCC3)nc(-n3ccc(C4COCCO4)n3)nc21] |
| 184 | 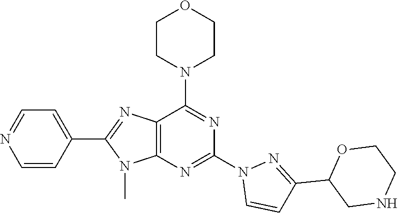 [SMILES: Cn1c(-c2ccncc2)nc2c(N3CCOCC3)nc(-n3ccc(C4CNCCO4)n3)nc21] |
| 185 | 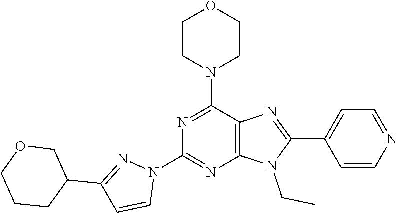 [SMILES: CCn1c(-c2ccncc2)nc2c(N3CCOCC3)nc(-n3ccc(C4CCCOC4)n3)nc21] |
| 186 | 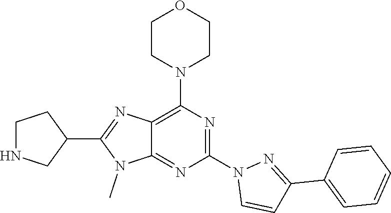 [SMILES: Cn1c(C2CCNC2)nc2c(N3CCOCC3)nc(-n3ccc(-c4ccccc4)n3)nc21] |
| 187 | 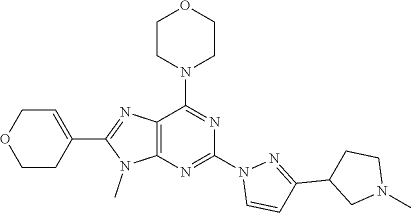 [SMILES: CN1CCC(c2ccn(-c3nc(N4CCOCC4)c4nc(C5=CCOCC5)n(C)c4n3)n2)C1] |
| 188 | 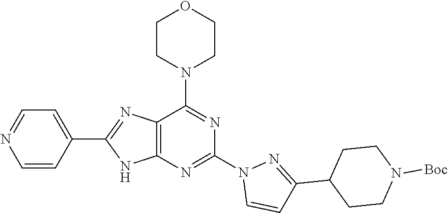 [SMILES: CC(C)(C)OC(=O)N1CCC(c2ccn(-c3nc(N4CCOCC4)c4nc(-c5ccncc5)[nH]c4n3)n2)CC1] |
| 189 | 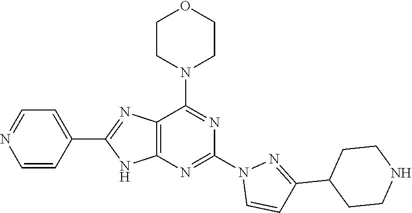 [SMILES: c1cc(-c2nc3c(N4CCOCC4)nc(-n4ccc(C5CCNCC5)n4)nc3[nH]2)ccn1] |
| 190 | 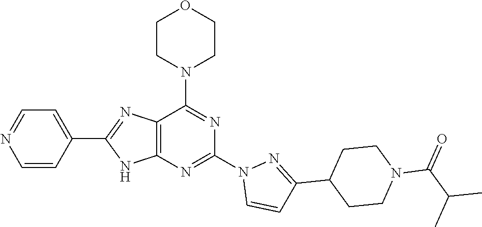 [SMILES: CC(C)C(=O)N1CCC(c2ccn(-c3nc(N4CCOCC4)c4nc(-c5ccncc5)[nH]c4n3)n2)CC1] |
| 191 | 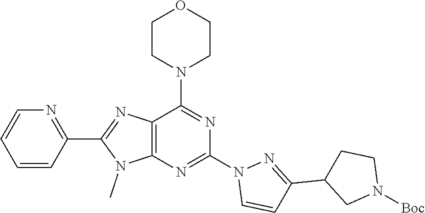 [SMILES: Cn1c(-c2ccccn2)nc2c(N3CCOCC3)nc(-n3ccc(C4CCN(C(=O)OC(C)(C)C)C4)n3)nc21] |
| 192 | 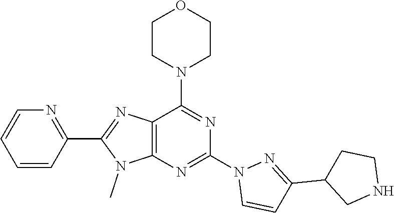 [SMILES: Cn1c(-c2ccccn2)nc2c(N3CCOCC3)nc(-n3ccc(C4CCNC4)n3)nc21] |
| 193 | 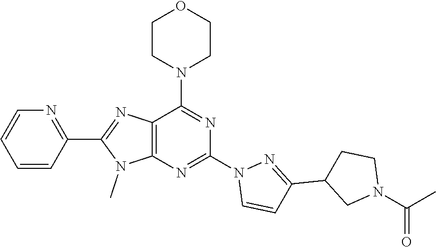 [SMILES: CC(=O)N1CCC(c2ccn(-c3nc(N4CCOCC4)c4nc(-c5ccccn5)n(C)c4n3)n2)C1] |
| 194 | 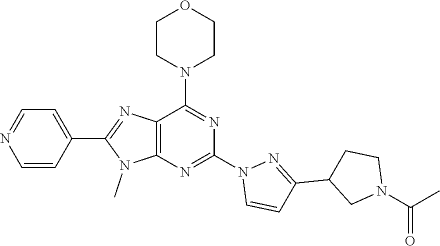 [SMILES: CC(=O)N1CCC(c2ccn(-c3nc(N4CCOCC4)c4nc(-c5ccncc5)n(C)c4n3)n2)C1] |
| 195 | 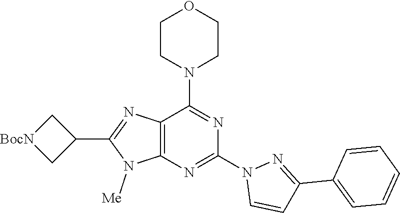 |
| 196 | 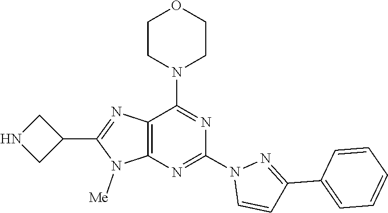 [SMILES: Cn1c(C2CNC2)nc2c(N3CCOCC3)nc(-n3ccc(-c4ccccc4)n3)nc21] |
| 197 | 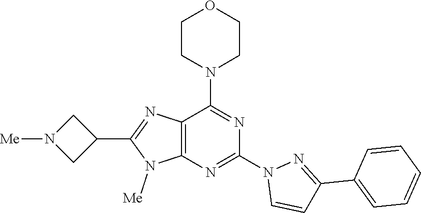 |
| 198 | 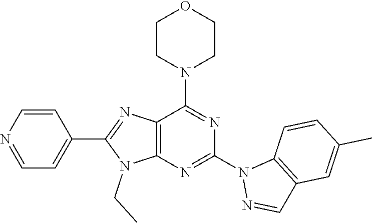 [SMILES: CCn1c(-c2ccncc2)nc2c(N3CCOCC3)nc(-n3ncc4cc(C)ccc43)nc21] |
| 199 | 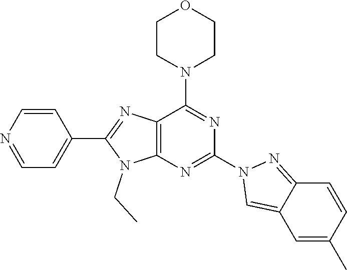 |
| 200 | 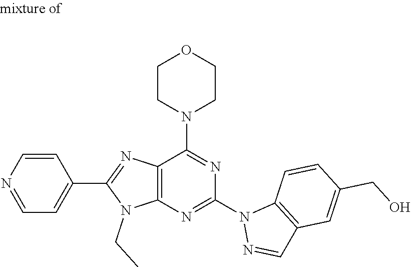 [SMILES: CCn1c(-c2ccncc2)nc2c(N3CCOCC3)nc(-n3ncc4cc(CO)ccc43)nc21] |
| | 200A |
| | 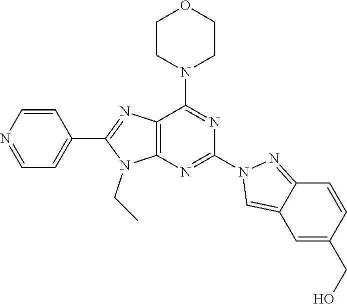 [SMILES: CCn1c(-c2ccncc2)nc2c(N3CCOCC3)nc(-n3cc4cc(CO)ccc4n3)nc21] |
| | 200B |
| 201 | mixture of |
| | 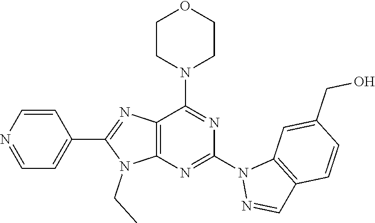 [SMILES: CCn1c(-c2ccncc2)nc2c(N3CCOCC3)nc(-n3ncc4ccc(CO)cc43)nc21] |
| | 201A |
| | 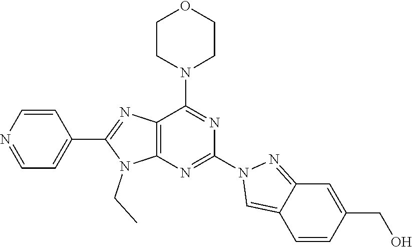 |
| | 201B |
| 202 | mixture of |
| | 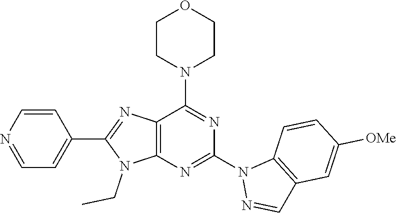 [SMILES: CCn1c(-c2ccncc2)nc2c(N3CCOCC3)nc(-n3ncc4cc(OC)ccc43)nc21] |
| | 202A |
| | 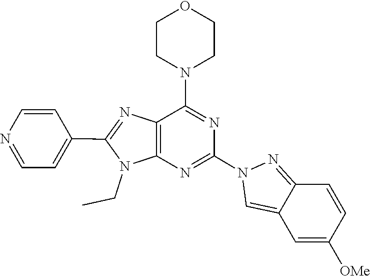 [SMILES: CCn1c(-c2ccncc2)nc2c(N3CCOCC3)nc(-n3cc4cc(OC)ccc4n3)nc21] |
| | 202B |
| 203 | 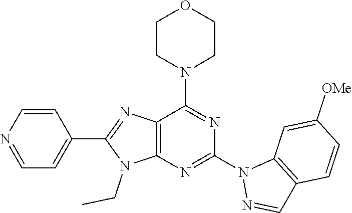 |
| 204 | 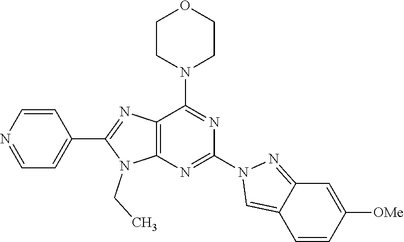 |
| 205 | 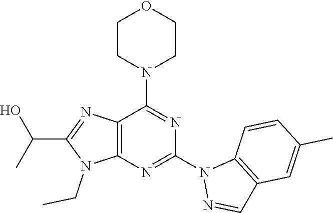 [SMILES: CCn1c(C(C)O)nc2c(N3CCOCC3)nc(-n3ncc4cc(C)ccc43)nc21] |
| 206 | 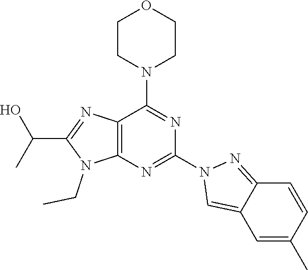 [SMILES: CCn1c(C(C)O)nc2c(N3CCOCC3)nc(-n3cc4cc(C)ccc4n3)nc21] |
| 207 | 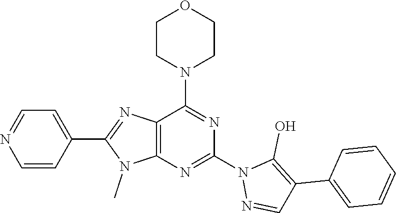 [SMILES: Cn1c(-c2ccncc2)nc2c(N3CCOCC3)nc(-n3ncc(-c4ccccc4)c3O)nc21] |
| 208 | 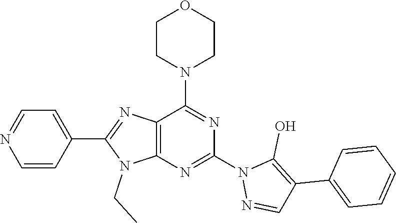 [SMILES: CCn1c(-c2ccncc2)nc2c(N3CCOCC3)nc(-n3ncc(-c4ccccc4)c3O)nc21] |
| 209 | 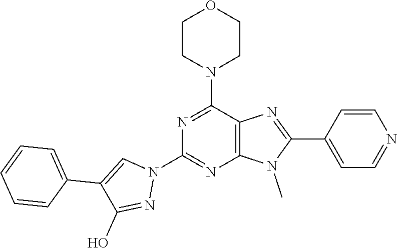 |
| 210 | 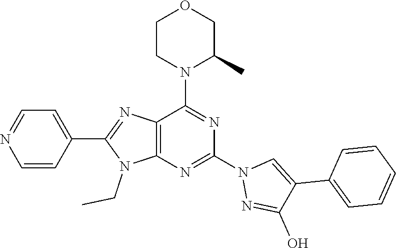 [SMILES: CCn1c(-c2ccncc2)nc2c(N3CCOC[C@H]3C)nc(-n3cc(-c4ccccc4)c(O)n3)nc21] |
| 211 | 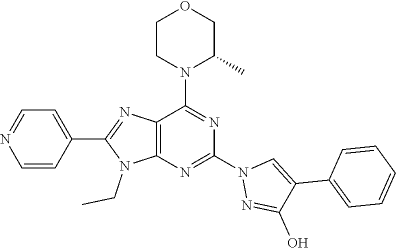 [SMILES: CCn1c(-c2ccncc2)nc2c(N3CCOC[C@@H]3C)nc(-n3cc(-c4ccccc4)c(O)n3)nc21] |
| 212 | 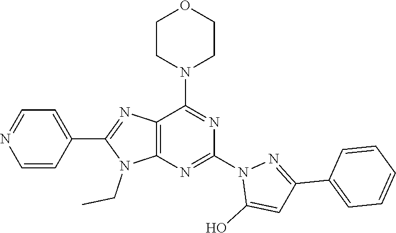 [SMILES: CCn1c(-c2ccncc2)nc2c(N3CCOCC3)nc(-n3nc(-c4ccccc4)cc3O)nc21] |
| 213 | 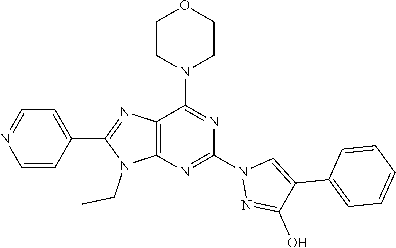 [SMILES: CCn1c(-c2ccncc2)nc2c(N3CCOCC3)nc(-n3cc(-c4ccccc4)c(O)n3)nc21] |
| 214 | 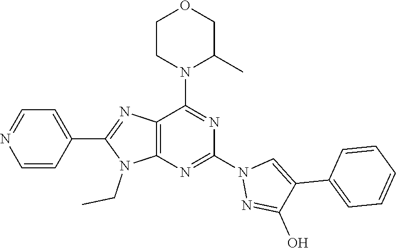 [SMILES: CCn1c(-c2ccncc2)nc2c(N3CCOCC3C)nc(-n3cc(-c4ccccc4)c(O)n3)nc21] |
| 215 | 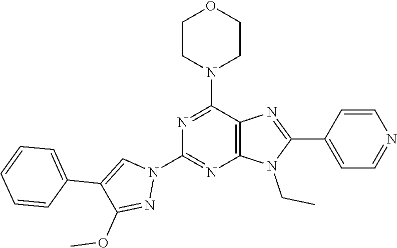 [SMILES: CCn1c(-c2ccncc2)nc2c(N3CCOCC3)nc(-n3cc(-c4ccccc4)c(OC)n3)nc21] |
| 216 | 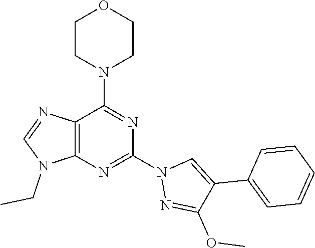 [SMILES: CCn1cnc2c(N3CCOCC3)nc(-n3cc(-c4ccccc4)c(OC)n3)nc21] |
| 217 | 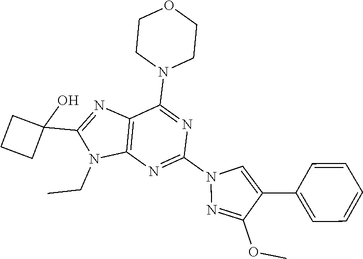 [SMILES: CCn1c(C2(O)CCC2)nc2c(N3CCOCC3)nc(-n3cc(-c4ccccc4)c(OC)n3)nc21] |
| 218 | 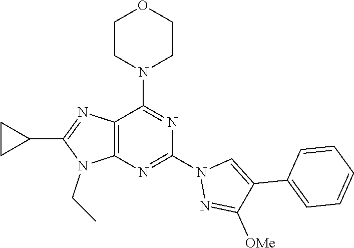 [SMILES: CCn1c(C2CC2)nc2c(N3CCOCC3)nc(-n3cc(-c4ccccc4)c(OC)n3)nc21] |
| 219 | 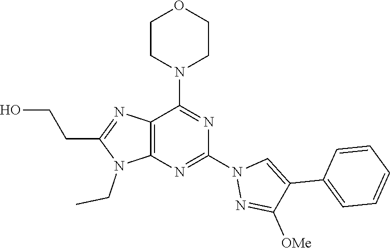 [SMILES: CCn1c(CCO)nc2c(N3CCOCC3)nc(-n3cc(-c4ccccc4)c(OC)n3)nc21] |
| 220 | 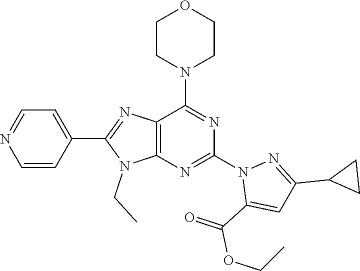 [SMILES: CCOC(=O)c1cc(C2CC2)nn1-c1nc(N2CCOCC2)c2nc(-c3ccncc3)n(CC)c2n1] |
| 221 | 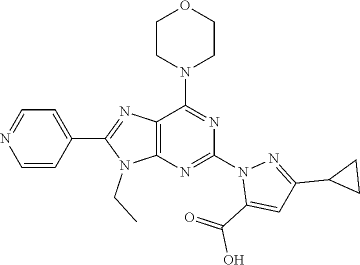 [SMILES: CCn1c(-c2ccncc2)nc2c(N3CCOCC3)nc(-n3nc(C4CC4)cc3C(=O)O)nc21] |
| 222 | 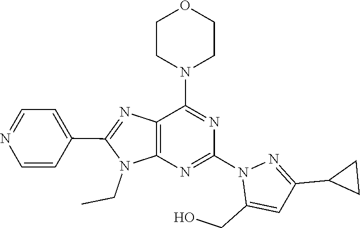 [SMILES: CCn1c(-c2ccncc2)nc2c(N3CCOCC3)nc(-n3nc(C4CC4)cc3CO)nc21] |
| 223 | 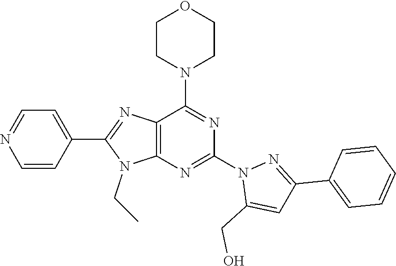 [SMILES: CCn1c(-c2ccncc2)nc2c(N3CCOCC3)nc(-n3nc(-c4ccccc4)cc3CO)nc21] |
| 224 | 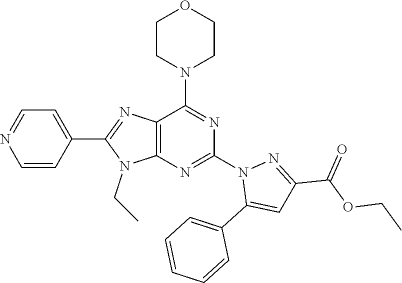 [SMILES: CCOC(=O)c1cc(-c2ccccc2)n(-c2nc(N3CCOCC3)c3nc(-c4ccncc4)n(CC)c3n2)n1] |
| 225 | 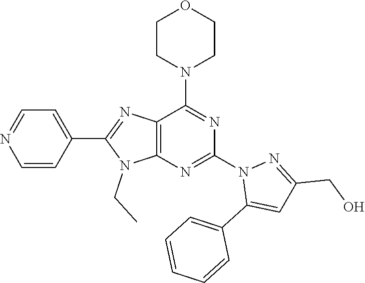 [SMILES: CCn1c(-c2ccncc2)nc2c(N3CCOCC3)nc(-n3nc(CO)cc3-c3ccccc3)nc21] |
| 226 | 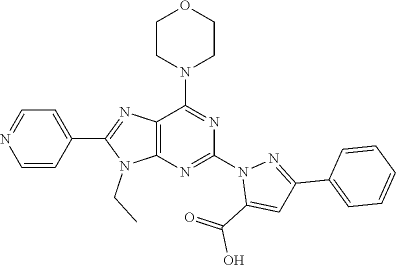 [SMILES: CCn1c(-c2ccncc2)nc2c(N3CCOCC3)nc(-n3nc(-c4ccccc4)cc3C(=O)O)nc21] |
| 227 | 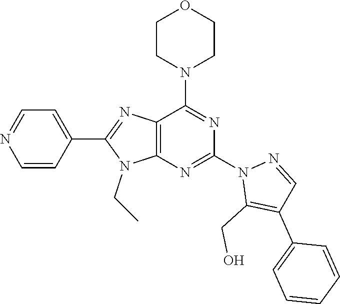 [SMILES: CCn1c(-c2ccncc2)nc2c(N3CCOCC3)nc(-n3ncc(-c4ccccc4)c3CO)nc21] |
| 228 | 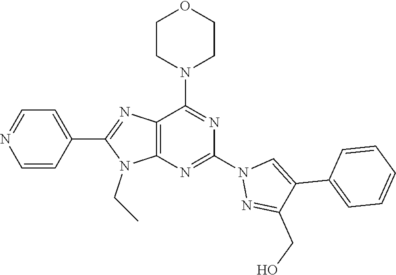 [SMILES: CCn1c(-c2ccncc2)nc2c(N3CCOCC3)nc(-n3cc(-c4ccccc4)c(CO)n3)nc21] |
| 229 | 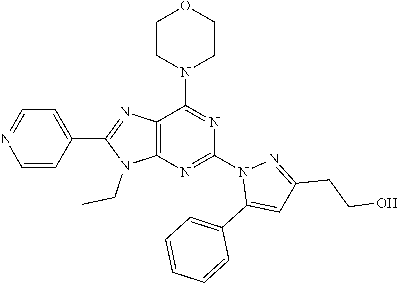 [SMILES: CCn1c(-c2ccncc2)nc2c(N3CCOCC3)nc(-n3nc(CCO)cc3-c3ccccc3)nc21] |
| 230 | 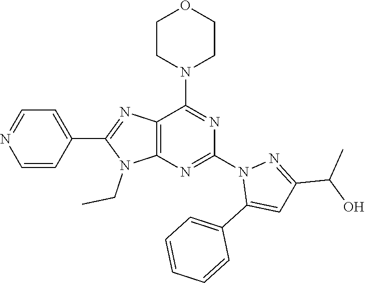 [SMILES: CCn1c(-c2ccncc2)nc2c(N3CCOCC3)nc(-n3nc(C(C)O)cc3-c3ccccc3)nc21] |
| 231 | 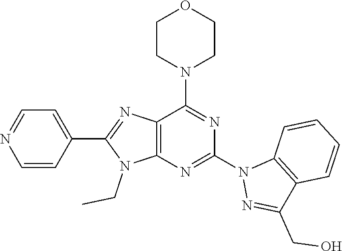 [SMILES: CCn1c(-c2ccncc2)nc2c(N3CCOCC3)nc(-n3nc(CO)c4ccccc43)nc21] |
| 232 | 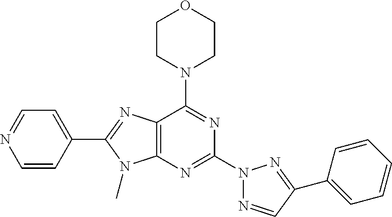 [SMILES: Cn1c(-c2ccncc2)nc2c(N3CCOCC3)nc(-n3ncc(-c4ccccc4)n3)nc21] |
| 233 | 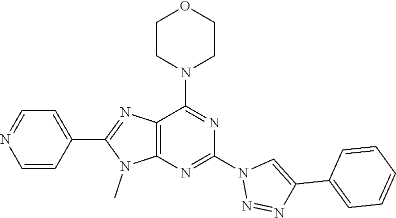 [SMILES: Cn1c(-c2ccncc2)nc2c(N3CCOCC3)nc(-n3cc(-c4ccccc4)nn3)nc21] |
| 234 | 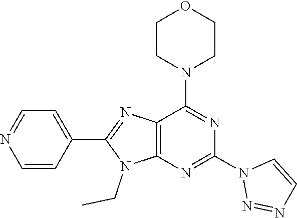 [SMILES: CCn1c(-c2ccncc2)nc2c(N3CCOCC3)nc(-n3ccnn3)nc21] |
| 235 | 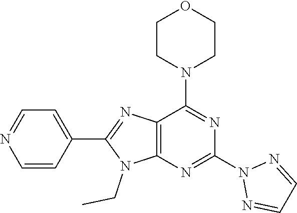 [SMILES: CCn1c(-c2ccncc2)nc2c(N3CCOCC3)nc(-n3nccn3)nc21] |
| 236 | 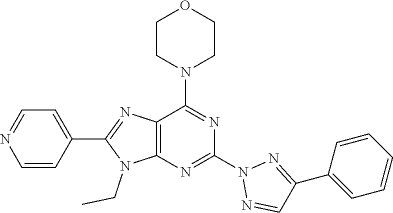 [SMILES: CCn1c(-c2ccncc2)nc2c(N3CCOCC3)nc(-n3ncc(-c4ccccc4)n3)nc21] |
| 237 | 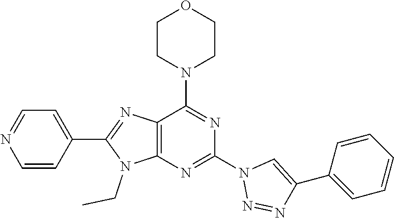 [SMILES: CCn1c(-c2ccncc2)nc2c(N3CCOCC3)nc(-n3cc(-c4ccccc4)nn3)nc21] |
| 238 | 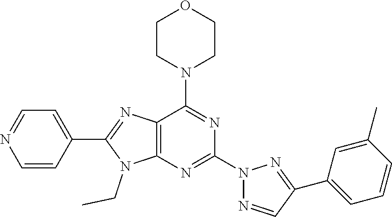 [SMILES: CCn1c(-c2ccncc2)nc2c(N3CCOCC3)nc(-n3ncc(-c4cccc(C)c4)n3)nc21] |
| 239 | 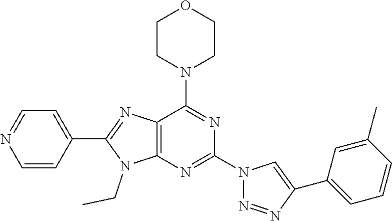 [SMILES: CCn1c(-c2ccncc2)nc2c(N3CCOCC3)nc(-n3cc(-c4cccc(C)c4)nn3)nc21] |
| 240 | 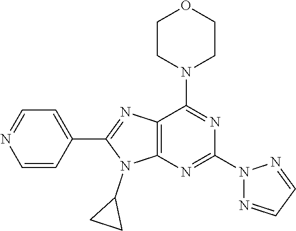 [SMILES: c1cc(-c2nc3c(N4CCOCC4)nc(-n4nccn4)nc3n2C2CC2)ccn1] |
| 241 | 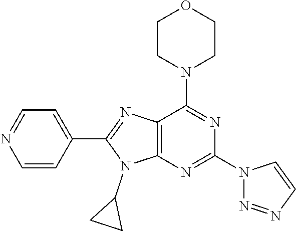 [SMILES: c1cc(-c2nc3c(N4CCOCC4)nc(-n4ccnn4)nc3n2C2CC2)ccn1] |
| 242 | 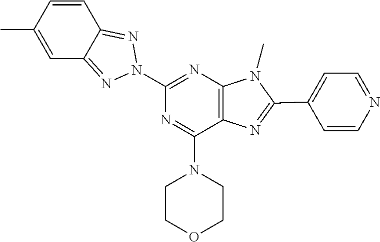 [SMILES: Cc1ccc2nn(-c3nc(N4CCOCC4)c4nc(-c5ccncc5)n(C)c4n3)nc2c1] |
| 243 | mixture of |
| | 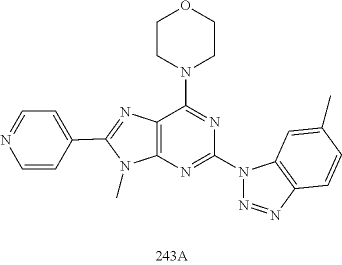 [SMILES: Cc1ccc2nnn(-c3nc(N4CCOCC4)c4nc(-c5ccncc5)n(C)c4n3)c2c1] |
| | 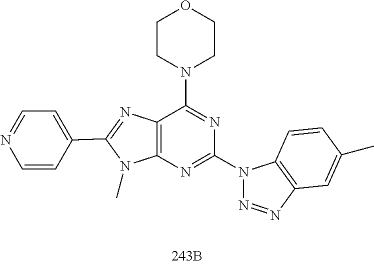 [SMILES: Cc1ccc2c(c1)nnn2-c1nc(N2CCOCC2)c2nc(-c3ccncc3)n(C)c2n1] |
| 244 | 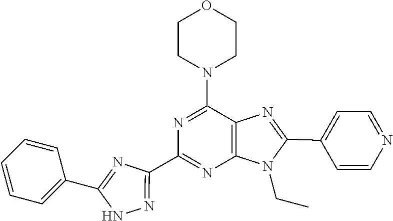 [SMILES: CCn1c(-c2ccncc2)nc2c(N3CCOCC3)nc(-c3n[nH]c(-c4ccccc4)n3)nc21] |
| 245 | 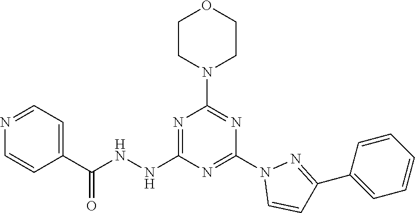 [SMILES: O=C(NNc1nc(N2CCOCC2)nc(-n2ccc(-c3ccccc3)n2)n1)c1ccncc1] |
| 246 | 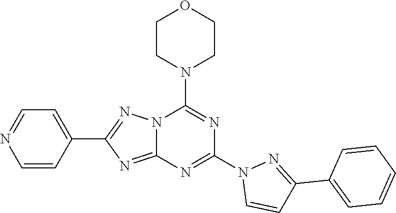 [SMILES: c1ccc(-c2ccn(-c3nc(N4CCOCC4)n4nc(-c5ccncc5)nc4n3)n2)cc1] |
| 247 | 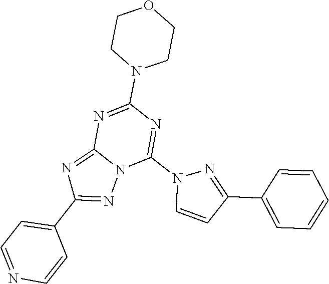 [SMILES: c1ccc(-c2ccn(-c3nc(N4CCOCC4)nc4nc(-c5ccncc5)nn34)n2)cc1] |
| 248 | 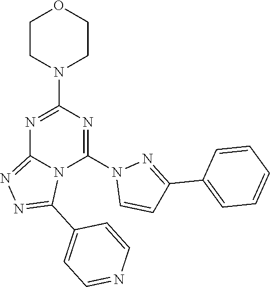 [SMILES: c1ccc(-c2ccn(-c3nc(N4CCOCC4)nc4nnc(-c5ccncc5)n34)n2)cc1] |
| 249 | 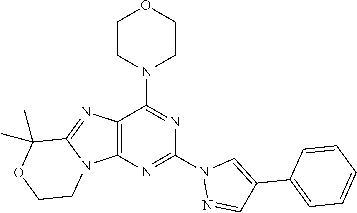 [SMILES: CC1(C)OCCn2c1nc1c(N3CCOCC3)nc(-n3cc(-c4ccccc4)cn3)nc12] |
| 250 | 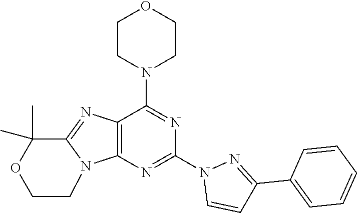 [SMILES: CC1(C)OCCn2c1nc1c(N3CCOCC3)nc(-n3ccc(-c4ccccc4)n3)nc12] |
| 251 | 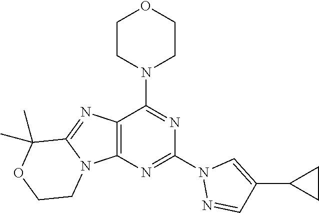 [SMILES: CC1(C)OCCn2c1nc1c(N3CCOCC3)nc(-n3cc(C4CC4)cn3)nc12] |
| 252 | 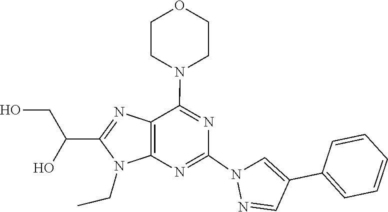 [SMILES: CCn1c(C(O)CO)nc2c(N3CCOCC3)nc(-n3cc(-c4ccccc4)cn3)nc21] |
| 253 | 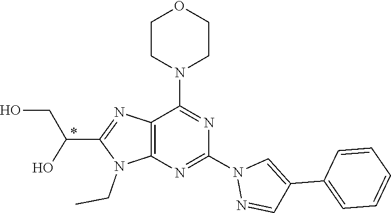 [SMILES: CCn1c(C(O)CO)nc2c(N3CCOCC3)nc(-n3cc(-c4ccccc4)cn3)nc21] |
| 254 | 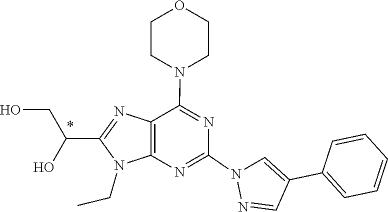 [SMILES: CCn1c(C(O)CO)nc2c(N3CCOCC3)nc(-n3cc(-c4ccccc4)cn3)nc21] |
| 255 | 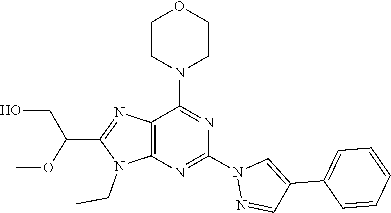 [SMILES: CCn1c(C(CO)OC)nc2c(N3CCOCC3)nc(-n3cc(-c4ccccc4)cn3)nc21] |
| 256 | 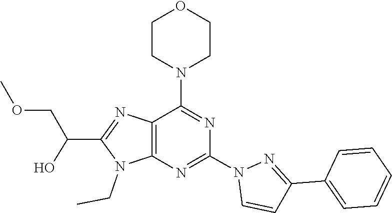 [SMILES: CCn1c(C(O)COC)nc2c(N3CCOCC3)nc(-n3ccc(-c4ccccc4)n3)nc21] |
| 257 | 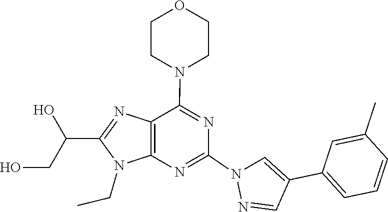 [SMILES: CCn1c(C(O)CO)nc2c(N3CCOCC3)nc(-n3cc(-c4cccc(C)c4)cn3)nc21] |
| 258 | 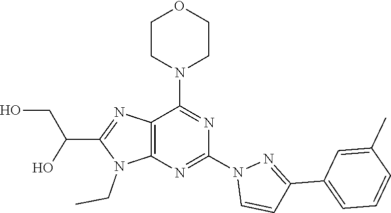 [SMILES: CCn1c(C(O)CO)nc2c(N3CCOCC3)nc(-n3ccc(-c4cccc(C)c4)n3)nc21] |
| 259 | 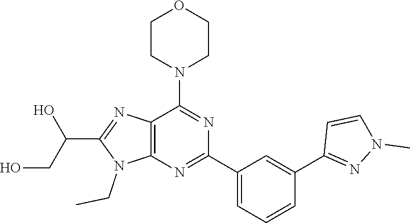 [SMILES: CCn1c(C(O)CO)nc2c(N3CCOCC3)nc(-c3cccc(-c4ccn(C)n4)c3)nc21] |
| 260 | 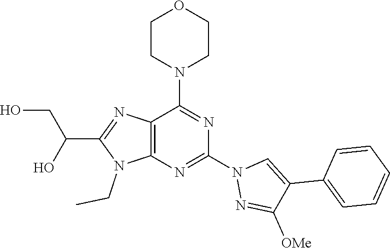 [SMILES: CCn1c(C(O)CO)nc2c(N3CCOCC3)nc(-n3cc(-c4ccccc4)c(OC)n3)nc21] |
| 261 | 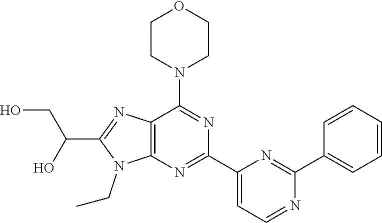 [SMILES: CCn1c(C(O)CO)nc2c(N3CCOCC3)nc(-c3ccnc(-c4ccccc4)n3)nc21] |
| 262 | 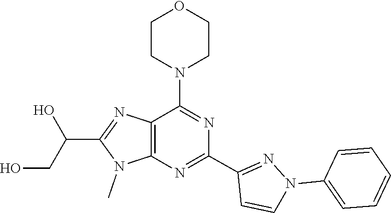 [SMILES: Cn1c(C(O)CO)nc2c(N3CCOCC3)nc(-c3ccn(-c4ccccc4)n3)nc21] |
| 263 | 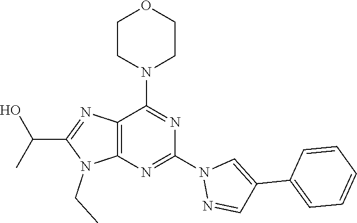 |
| 264 | 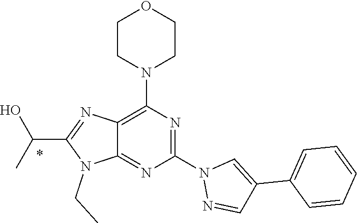 [SMILES: CCn1c(C(C)O)nc2c(N3CCOCC3)nc(-n3cc(-c4ccccc4)cn3)nc21] |
| 265 | 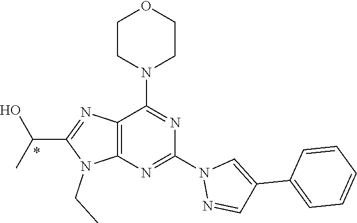 [SMILES: CCn1c(C(C)O)nc2c(N3CCOCC3)nc(-n3cc(-c4ccccc4)cn3)nc21] |
| 266 | 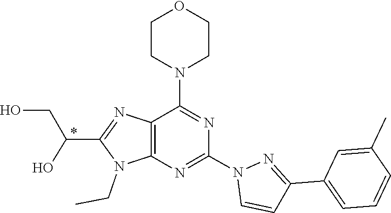 [SMILES: CCn1c(C(O)CO)nc2c(N3CCOCC3)nc(-n3ccc(-c4cccc(C)c4)n3)nc21] |
| 267 | 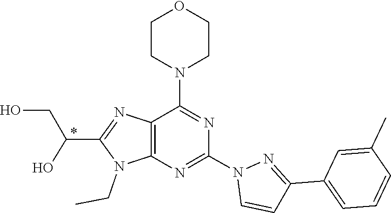 [SMILES: CCn1c(C(O)CO)nc2c(N3CCOCC3)nc(-n3ccc(-c4cccc(C)c4)n3)nc21] |
| 268 | 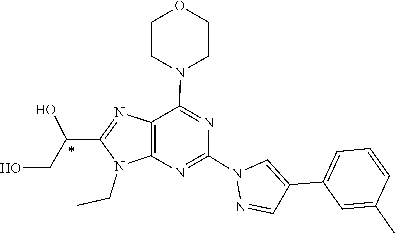 [SMILES: CCn1c(C(O)CO)nc2c(N3CCOCC3)nc(-n3cc(-c4cccc(C)c4)cn3)nc21] |
| 269 | 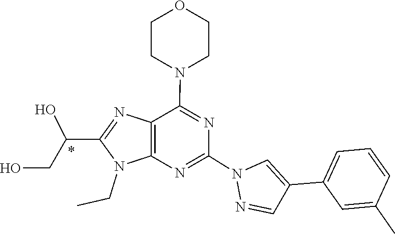 [SMILES: CCn1c(C(O)CO)nc2c(N3CCOCC3)nc(-n3cc(-c4cccc(C)c4)cn3)nc21] |
| 270 | 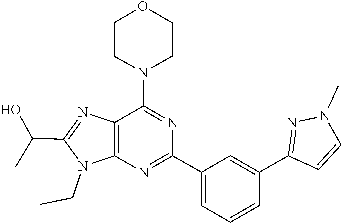 |
| 271 | 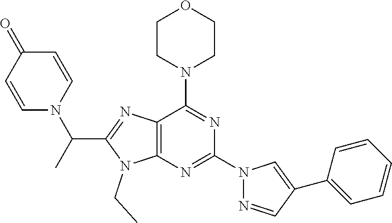 [SMILES: CCn1c(C(C)n2ccc(=O)cc2)nc2c(N3CCOCC3)nc(-n3cc(-c4ccccc4)cn3)nc21] |
| 272 | 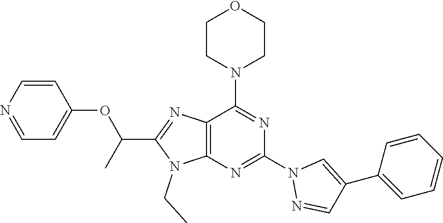 [SMILES: CCn1c(C(C)Oc2ccncc2)nc2c(N3CCOCC3)nc(-n3cc(-c4ccccc4)cn3)nc21] |
| 273 | 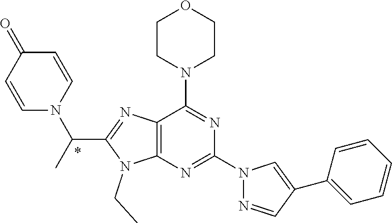 [SMILES: CCn1c(C(C)n2ccc(=O)cc2)nc2c(N3CCOCC3)nc(-n3cc(-c4ccccc4)cn3)nc21] |
| 274 | 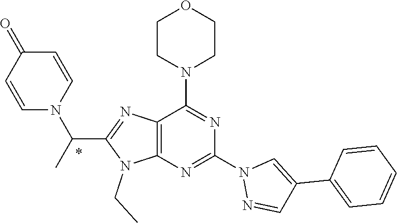 [SMILES: CCn1c(C(C)n2ccc(=O)cc2)nc2c(N3CCOCC3)nc(-n3cc(-c4ccccc4)cn3)nc21] |
| 275 | 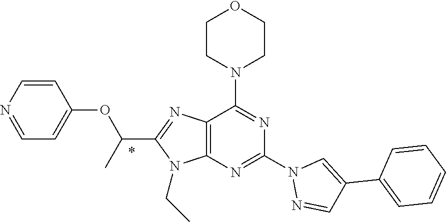 [SMILES: CCn1c(C(C)Oc2ccncc2)nc2c(N3CCOCC3)nc(-n3cc(-c4ccccc4)cn3)nc21] |
| 276 | 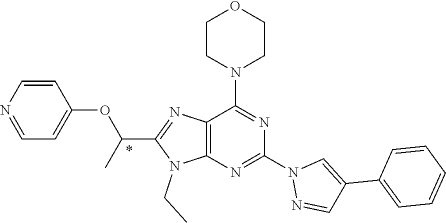 [SMILES: CCn1c(C(C)Oc2ccncc2)nc2c(N3CCOCC3)nc(-n3cc(-c4ccccc4)cn3)nc21] |
| 277 | 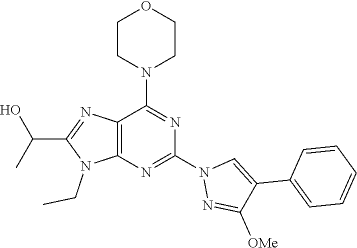 [SMILES: CCn1c(C(C)O)nc2c(N3CCOCC3)nc(-n3cc(-c4ccccc4)c(OC)n3)nc21] |
| 278 | 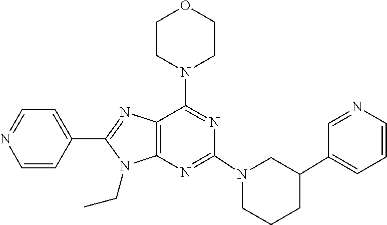 [SMILES: CCn1c(-c2ccncc2)nc2c(N3CCOCC3)nc(N3CCCC(c4cccnc4)C3)nc21] |
| 279 | 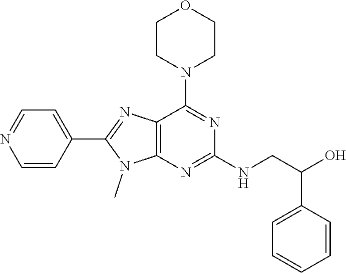 [SMILES: Cn1c(-c2ccncc2)nc2c(N3CCOCC3)nc(NCC(O)c3ccccc3)nc21] |
| 280 | 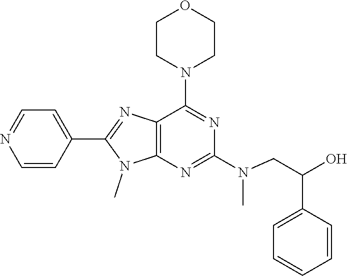 [SMILES: CN(CC(O)c1ccccc1)c1nc(N2CCOCC2)c2nc(-c3ccncc3)n(C)c2n1] |
| 281 | 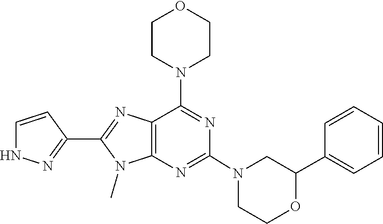 [SMILES: Cn1c(-c2cc[nH]n2)nc2c(N3CCOCC3)nc(N3CCOC(c4ccccc4)C3)nc21] |
| 282 | 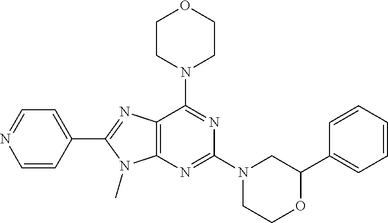 [SMILES: Cn1c(-c2ccncc2)nc2c(N3CCOCC3)nc(N3CCOC(c4ccccc4)C3)nc21] |
| 283 | 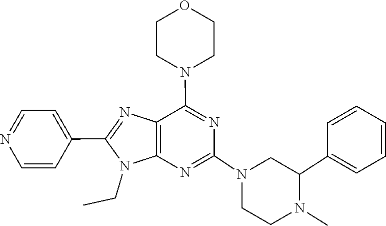 [SMILES: CCn1c(-c2ccncc2)nc2c(N3CCOCC3)nc(N3CCN(C)C(c4ccccc4)C3)nc21] |
| 284 | 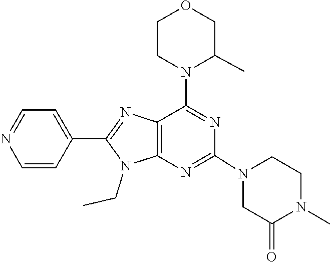 [SMILES: CCn1c(-c2ccncc2)nc2c(N3CCOCC3C)nc(N3CCN(C)C(=O)C3)nc21] |
| 285 | 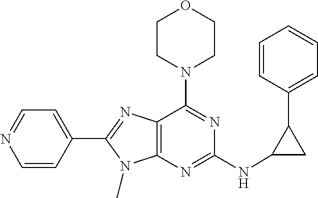 [SMILES: Cn1c(-c2ccncc2)nc2c(N3CCOCC3)nc(NC3CC3c3ccccc3)nc21] |
| 286 | 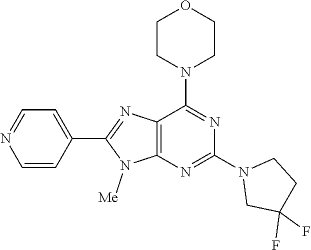 [SMILES: Cn1c(-c2ccncc2)nc2c(N3CCOCC3)nc(N3CCC(F)(F)C3)nc21] |
| 287 | 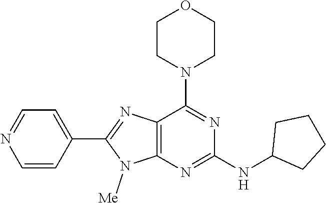 [SMILES: Cn1c(-c2ccncc2)nc2c(N3CCOCC3)nc(NC3CCCC3)nc21] |
| 288 | 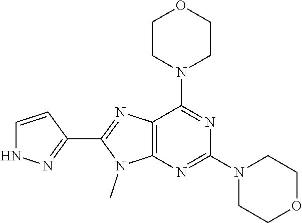 [SMILES: Cn1c(-c2cc[nH]n2)nc2c(N3CCOCC3)nc(N3CCOCC3)nc21] |
| 289 | 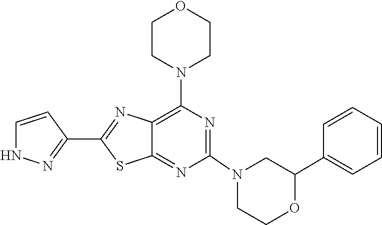 [SMILES: c1ccc(C2CN(c3nc(N4CCOCC4)c4nc(-c5cc[nH]n5)sc4n3)CCO2)cc1] |
| 290 | 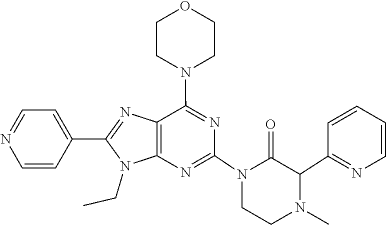 [SMILES: CCn1c(-c2ccncc2)nc2c(N3CCOCC3)nc(N3CCN(C)C(c4ccccn4)C3=O)nc21] |
| 291 | 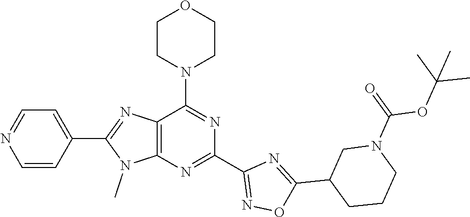 [SMILES: Cn1c(-c2ccncc2)nc2c(N3CCOCC3)nc(-c3noc(C4CCCN(C(=O)OC(C)(C)C)C4)n3)nc21] |
| 292 | 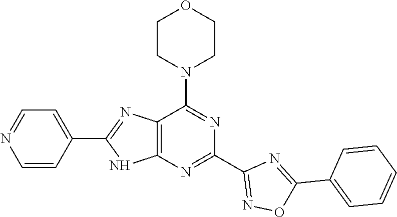 [SMILES: c1ccc(-c2nc(-c3nc(N4CCOCC4)c4nc(-c5ccncc5)[nH]c4n3)no2)cc1] |
| 293 | 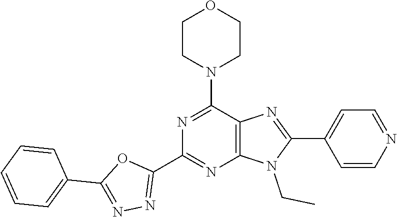 [SMILES: CCn1c(-c2ccncc2)nc2c(N3CCOCC3)nc(-c3nnc(-c4ccccc4)o3)nc21] |
| 294 | 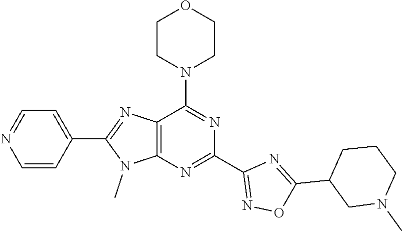 [SMILES: CN1CCCC(c2nc(-c3nc(N4CCOCC4)c4nc(-c5ccncc5)n(C)c4n3)no2)C1] |
| 295 | 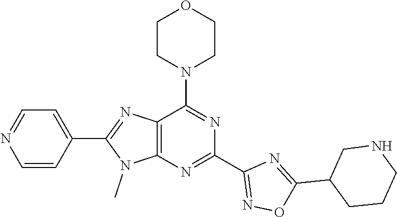 [SMILES: Cn1c(-c2ccncc2)nc2c(N3CCOCC3)nc(-c3noc(C4CCCNC4)n3)nc21] |
| 296 | 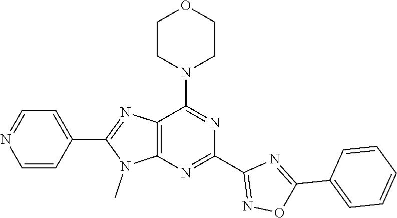 [SMILES: Cn1c(-c2ccncc2)nc2c(N3CCOCC3)nc(-c3noc(-c4ccccc4)n3)nc21] |
| 297 | 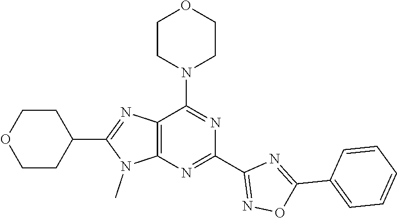 [SMILES: Cn1c(C2CCOCC2)nc2c(N3CCOCC3)nc(-c3noc(-c4ccccc4)n3)nc21] |
| 298 | 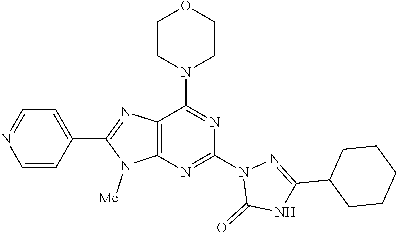 [SMILES: Cn1c(-c2ccncc2)nc2c(N3CCOCC3)nc(-n3nc(C4CCCCC4)[nH]c3=O)nc21] |
| 299 | 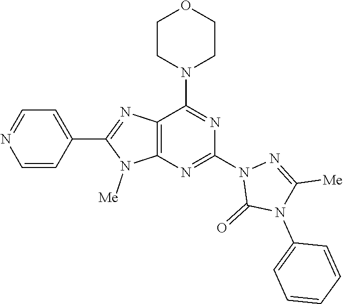 [SMILES: Cc1nn(-c2nc(N3CCOCC3)c3nc(-c4ccncc4)n(C)c3n2)c(=O)n1-c1ccccc1] |
| 300 | 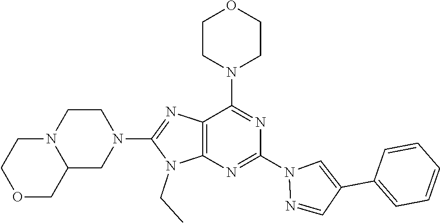 [SMILES: CCn1c(N2CCN3CCOCC3C2)nc2c(N3CCOCC3)nc(-n3cc(-c4ccccc4)cn3)nc21] |
| 301 | 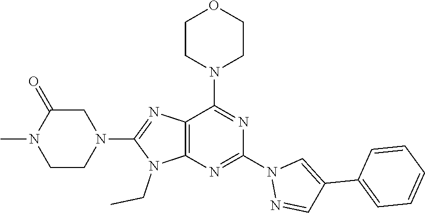 [SMILES: CCn1c(N2CCN(C)C(=O)C2)nc2c(N3CCOCC3)nc(-n3cc(-c4ccccc4)cn3)nc21] |
| 302 | 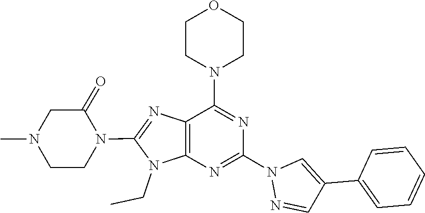 [SMILES: CCn1c(N2CCN(C)CC2=O)nc2c(N3CCOCC3)nc(-n3cc(-c4ccccc4)cn3)nc21] |
| 303 | 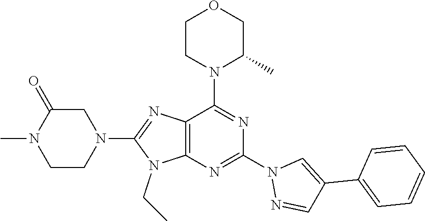 |
| 304 | 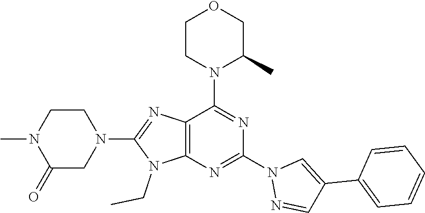 [SMILES: CCn1c(N2CCN(C)C(=O)C2)nc2c(N3CCOC[C@H]3C)nc(-n3cc(-c4ccccc4)cn3)nc21] |
| 305 | 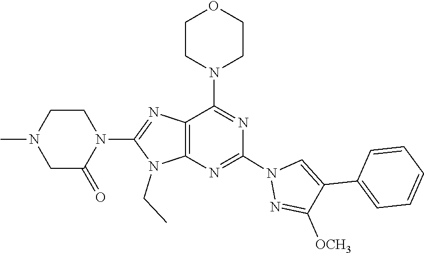 [SMILES: CCn1c(N2CCN(C)CC2=O)nc2c(N3CCOCC3)nc(-n3cc(-c4ccccc4)c(OC)n3)nc21] |
| 306 | 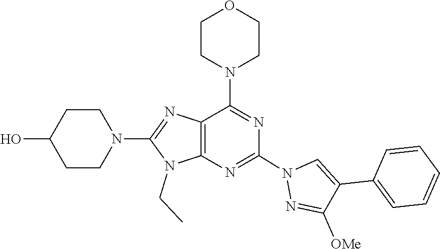 [SMILES: CCn1c(N2CCC(O)CC2)nc2c(N3CCOCC3)nc(-n3cc(-c4ccccc4)c(OC)n3)nc21] |
| 307 | 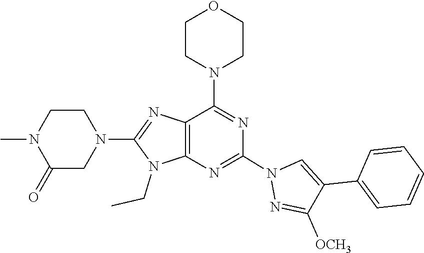 [SMILES: CCn1c(N2CCN(C)C(=O)C2)nc2c(N3CCOCC3)nc(-n3cc(-c4ccccc4)c(OC)n3)nc21] |
| 308 | 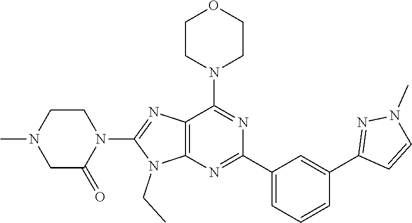 [SMILES: CCn1c(N2CCN(C)CC2=O)nc2c(N3CCOCC3)nc(-c3cccc(-c4ccn(C)n4)c3)nc21] |
| 309 | 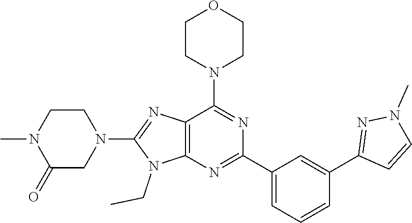 [SMILES: CCn1c(N2CCN(C)C(=O)C2)nc2c(N3CCOCC3)nc(-c3cccc(-c4ccn(C)n4)c3)nc21] |
| 310 | 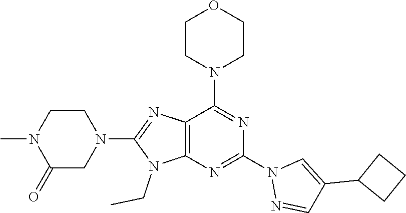 [SMILES: CCn1c(N2CCN(C)C(=O)C2)nc2c(N3CCOCC3)nc(-n3cc(C4CCC4)cn3)nc21] |
| 311 | 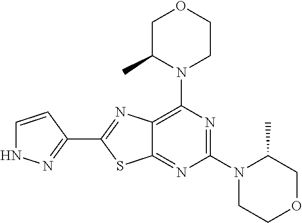 [SMILES: C[C@@H]1COCCN1c1nc(N2CCOC[C@@H]2C)c2nc(-c3cc[nH]n3)sc2n1] |
| 312 | 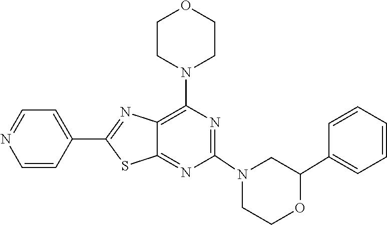 [SMILES: c1ccc(C2CN(c3nc(N4CCOCC4)c4nc(-c5ccncc5)sc4n3)CCO2)cc1] |
| 313 | 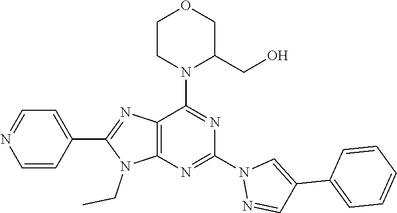 [SMILES: CCn1c(-c2ccncc2)nc2c(N3CCOCC3CO)nc(-n3cc(-c4ccccc4)cn3)nc21] |
| 314 | 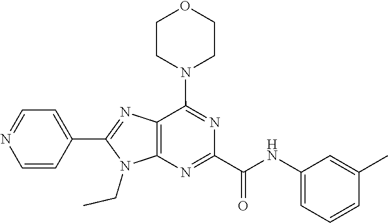 [SMILES: CCn1c(-c2ccncc2)nc2c(N3CCOCC3)nc(C(=O)Nc3cccc(C)c3)nc21] |
| 315 | 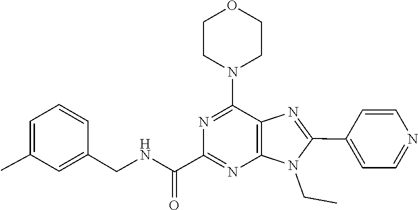 [SMILES: CCn1c(-c2ccncc2)nc2c(N3CCOCC3)nc(C(=O)NCc3cccc(C)c3)nc21] |
| 316 | 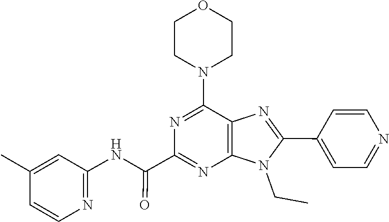 [SMILES: CCn1c(-c2ccncc2)nc2c(N3CCOCC3)nc(C(=O)Nc3cc(C)ccn3)nc21] |
| 317 | 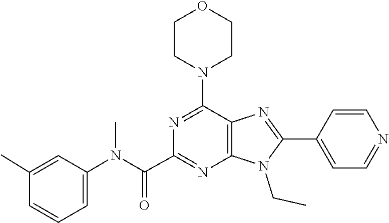 [SMILES: CCn1c(-c2ccncc2)nc2c(N3CCOCC3)nc(C(=O)N(C)c3cccc(C)c3)nc21] |
| 318 | 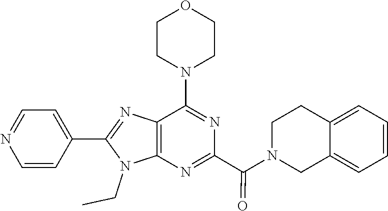 [SMILES: CCn1c(-c2ccncc2)nc2c(N3CCOCC3)nc(C(=O)N3CCc4ccccc4C3)nc21] |
| 319 | 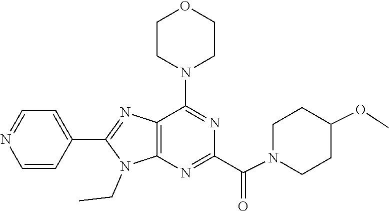 |
| 320 | 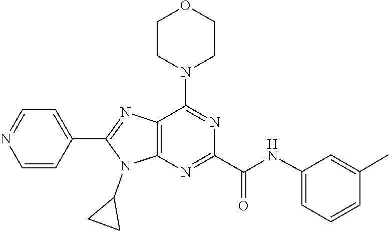 [SMILES: Cc1cccc(NC(=O)c2nc(N3CCOCC3)c3nc(-c4ccncc4)n(C4CC4)c3n2)c1] |
| 321 | 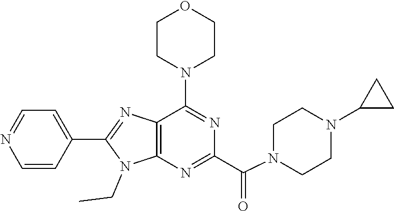 [SMILES: CCn1c(-c2ccncc2)nc2c(N3CCOCC3)nc(C(=O)N3CCN(C4CC4)CC3)nc21] |
| 322 | 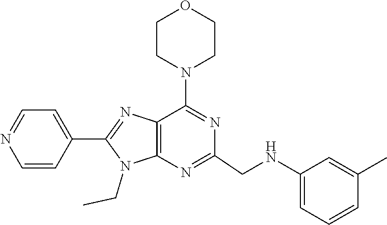 [SMILES: CCn1c(-c2ccncc2)nc2c(N3CCOCC3)nc(CNc3cccc(C)c3)nc21] |
| 323 | 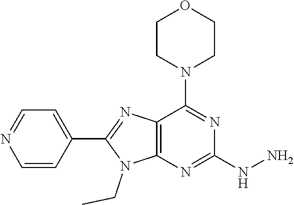 [SMILES: CCn1c(-c2ccncc2)nc2c(N3CCOCC3)nc(NN)nc21] |
| 324 | 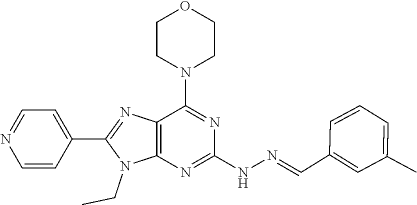 [SMILES: CCn1c(-c2ccncc2)nc2c(N3CCOCC3)nc(N/N=C/c3cccc(C)c3)nc21] |
| 325 | 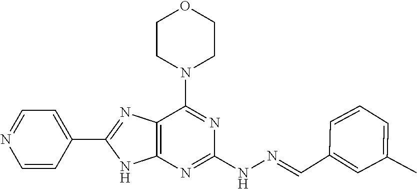 [SMILES: Cc1cccc(/C=N/Nc2nc(N3CCOCC3)c3nc(-c4ccncc4)[nH]c3n2)c1] |
| 326 | 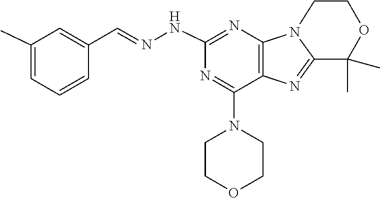 |
| 327 | 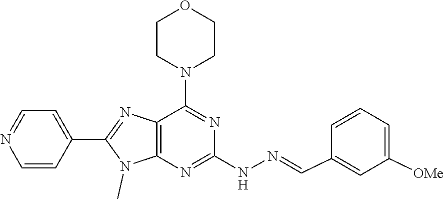 [SMILES: COc1cccc(/C=N/Nc2nc(N3CCOCC3)c3nc(-c4ccncc4)n(C)c3n2)c1] |
| 328 | 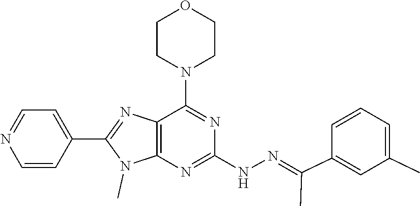 [SMILES: C/C(=N\Nc1nc(N2CCOCC2)c2nc(-c3ccncc3)n(C)c2n1)c1cccc(C)c1] |
| 329 | 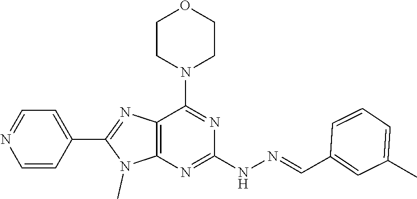 [SMILES: Cc1cccc(/C=N/Nc2nc(N3CCOCC3)c3nc(-c4ccncc4)n(C)c3n2)c1] |
| 330 | 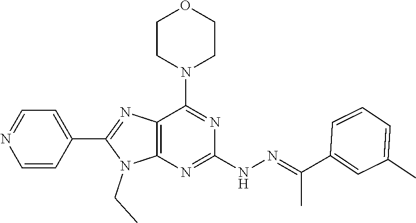 [SMILES: CCn1c(-c2ccncc2)nc2c(N3CCOCC3)nc(N/N=C(\C)c3cccc(C)c3)nc21] |
| 331 | 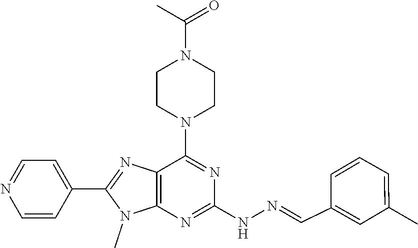 [SMILES: CC(=O)N1CCN(c2nc(N/N=C/c3cccc(C)c3)nc3c2nc(-c2ccncc2)n3C)CC1] |
| 332 | 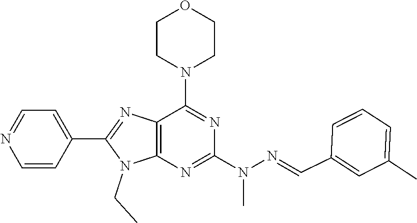 [SMILES: CCn1c(-c2ccncc2)nc2c(N3CCOCC3)nc(N(C)/N=C/c3cccc(C)c3)nc21] |
| 333 | 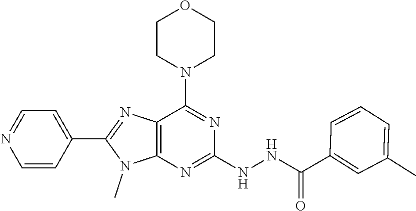 [SMILES: Cc1cccc(C(=O)NNc2nc(N3CCOCC3)c3nc(-c4ccncc4)n(C)c3n2)c1] |
| 334 | 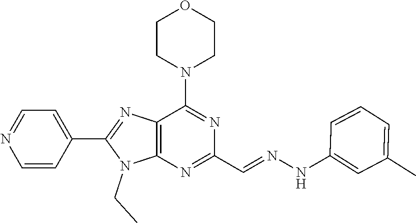 [SMILES: CCn1c(-c2ccncc2)nc2c(N3CCOCC3)nc(/C=N/Nc3cccc(C)c3)nc21] |
| 335 | 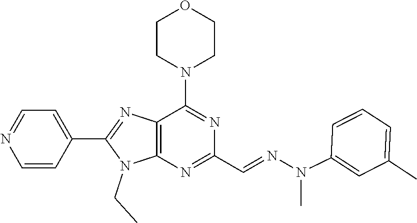 [SMILES: CCn1c(-c2ccncc2)nc2c(N3CCOCC3)nc(/C=N/N(C)c3cccc(C)c3)nc21] |
| 336 | 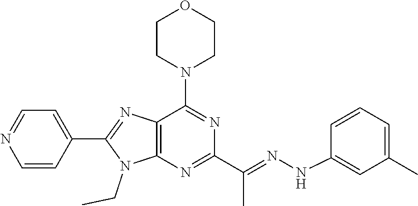 [SMILES: CCn1c(-c2ccncc2)nc2c(N3CCOCC3)nc(/C(C)=N/Nc3cccc(C)c3)nc21] |
| 337 | 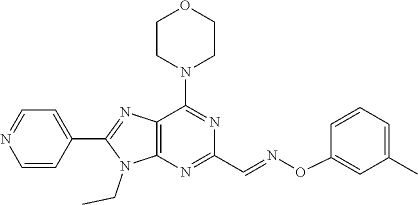 [SMILES: CCn1c(-c2ccncc2)nc2c(N3CCOCC3)nc(/C=N/Oc3cccc(C)c3)nc21] |
| 338 | 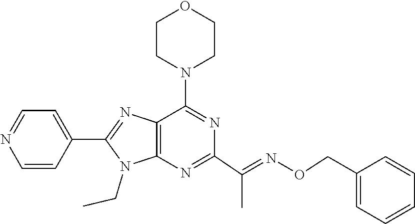 [SMILES: CCn1c(-c2ccncc2)nc2c(N3CCOCC3)nc(/C(C)=N/OCc3ccccc3)nc21] |
| 339 | 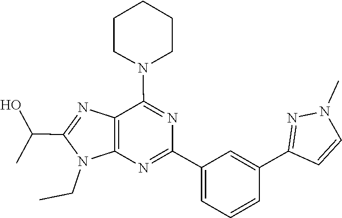 |
| 340 | 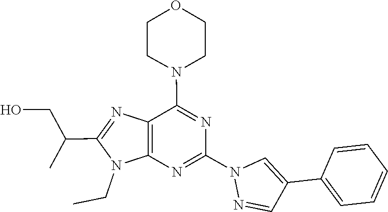 |
| 341 | 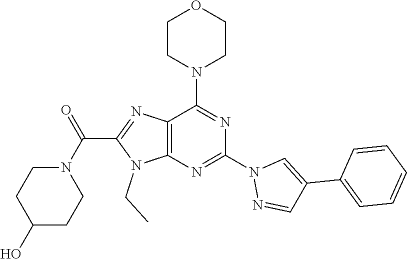 [SMILES: CCn1c(C(=O)N2CCC(O)CC2)nc2c(N3CCOCC3)nc(-n3cc(-c4ccccc4)cn3)nc21] |
| 342 | 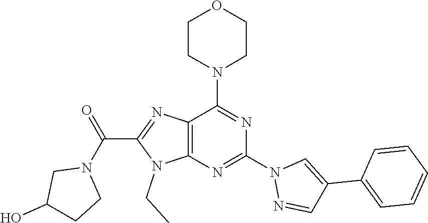 [SMILES: CCn1c(C(=O)N2CCC(O)C2)nc2c(N3CCOCC3)nc(-n3cc(-c4ccccc4)cn3)nc21] |
| 343 | 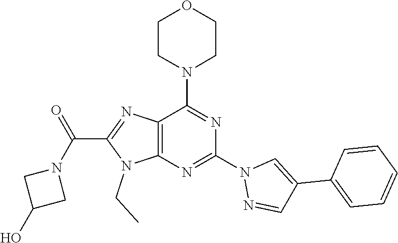 [SMILES: CCn1c(C(=O)N2CC(O)C2)nc2c(N3CCOCC3)nc(-n3cc(-c4ccccc4)cn3)nc21] |
| 344 | 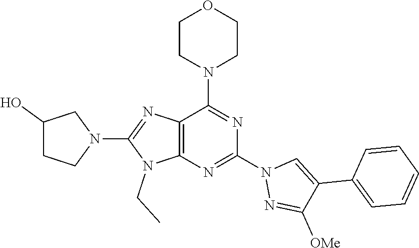 |
| 345 | 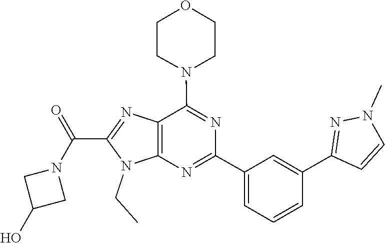 |
| 346 | 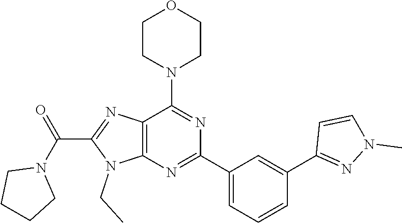 [SMILES: CCn1c(C(=O)N2CCCC2)nc2c(N3CCOCC3)nc(-c3cccc(-c4ccn(C)n4)c3)nc21] |
| 347 | 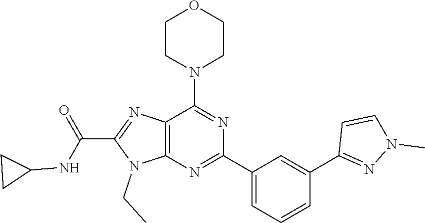 [SMILES: CCn1c(C(=O)NC2CC2)nc2c(N3CCOCC3)nc(-c3cccc(-c4ccn(C)n4)c3)nc21] |
| 348 | 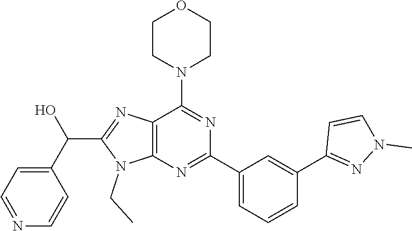 |
| 349 | 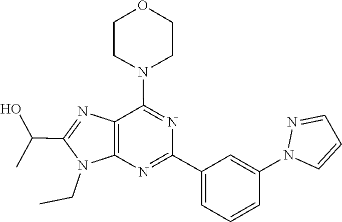 |
| 350 | 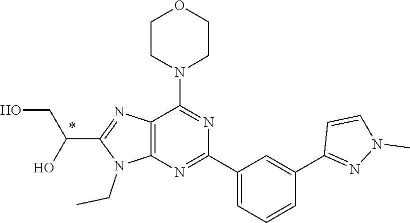 [SMILES: CCn1c(C(O)CO)nc2c(N3CCOCC3)nc(-c3cccc(-c4ccn(C)n4)c3)nc21] |
| 351 | 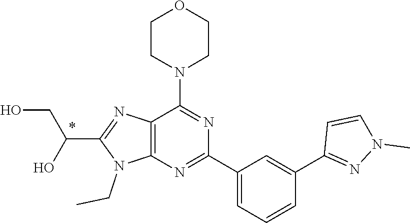 [SMILES: CCn1c(C(O)CO)nc2c(N3CCOCC3)nc(-c3cccc(-c4ccn(C)n4)c3)nc21] |
| 352 | 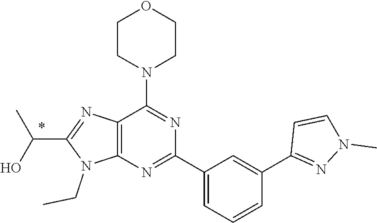 |
| 353 | 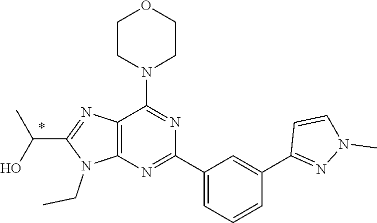 |
| 354 | 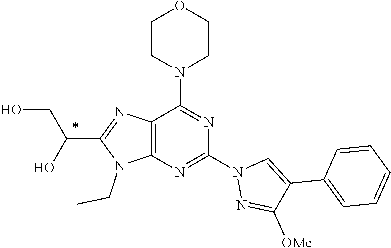 |
| 355 | 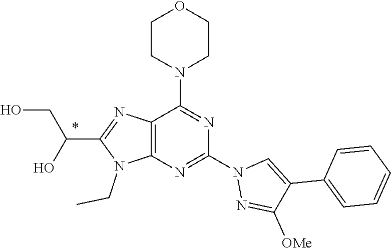 |
| 356 | 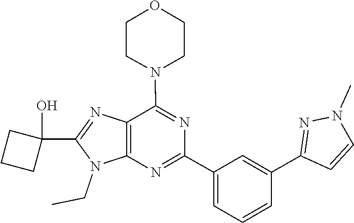 |
| 357 | 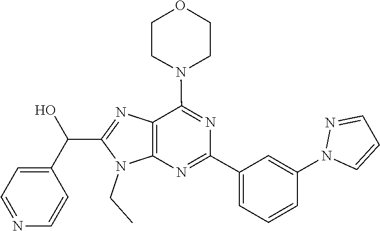 |
| 358 | 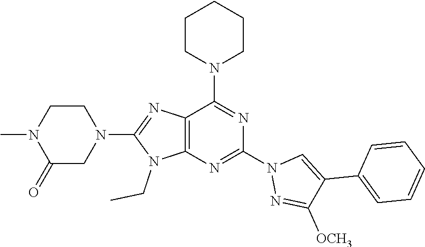 [SMILES: CCn1c(N2CCN(C)C(=O)C2)nc2c(N3CCCCC3)nc(-n3cc(-c4ccccc4)c(OC)n3)nc21] |
| 359 | 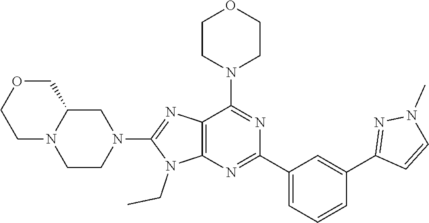 [SMILES: CCn1c(N2CCN3CCOC[C@@H]3C2)nc2c(N3CCOCC3)nc(-c3cccc(-c4ccn(C)n4)c3)nc21] |
| 360 | 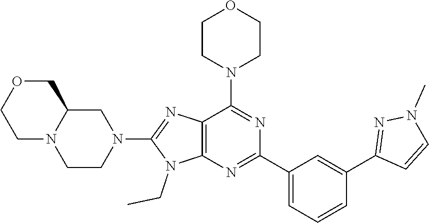 [SMILES: CCn1c(N2CCN3CCOC[C@H]3C2)nc2c(N3CCOCC3)nc(-c3cccc(-c4ccn(C)n4)c3)nc21] |
| 361 | 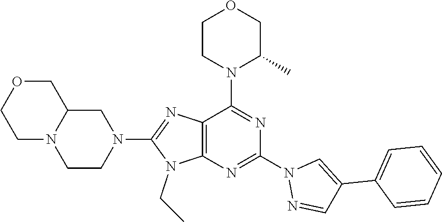 [SMILES: CCn1c(N2CCN3CCOCC3C2)nc2c(N3CCOC[C@@H]3C)nc(-n3cc(-c4ccccc4)cn3)nc21] |
| 362 | 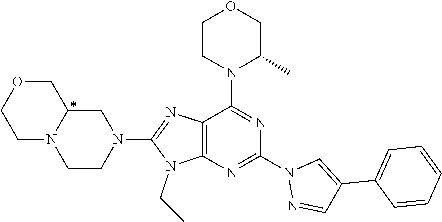 |
| 363 | 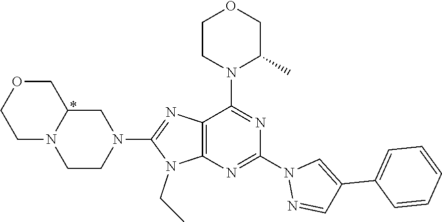 |
| 364 | 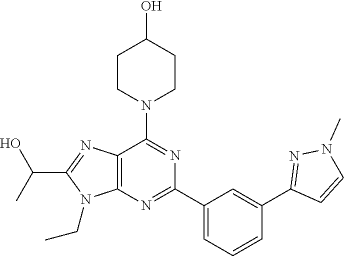 [SMILES: CCn1c(C(C)O)nc2c(N3CCC(O)CC3)nc(-c3cccc(-c4ccn(C)n4)c3)nc21] |
| 365 | 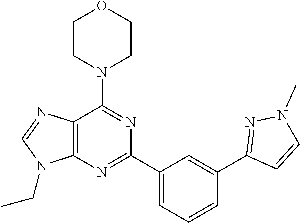 [SMILES: CCn1cnc2c(N3CCOCC3)nc(-c3cccc(-c4ccn(C)n4)c3)nc21] |
| 366 | 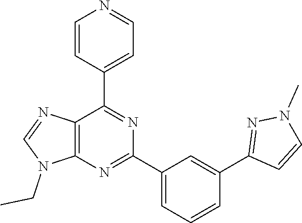 [SMILES: CCn1cnc2c(-c3ccncc3)nc(-c3cccc(-c4ccn(C)n4)c3)nc21] |
| 367 | 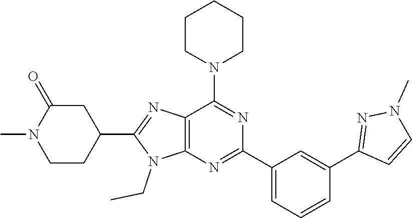 |
| 368 | 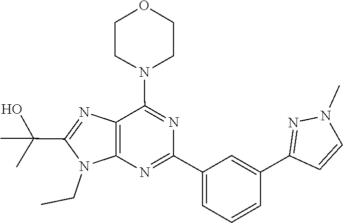 |
| 369 | 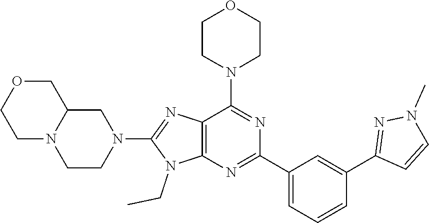 |
| 370 | 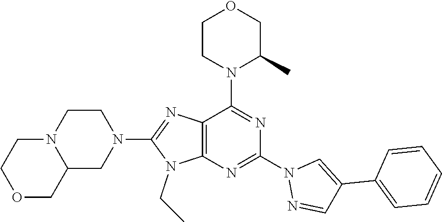 |
| 371 | 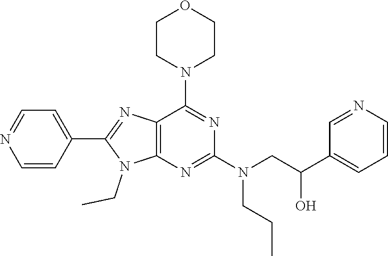 [SMILES: CCCN(CC(O)c1cccnc1)c1nc(N2CCOCC2)c2nc(-c3ccncc3)n(CC)c2n1] |
| 372 | 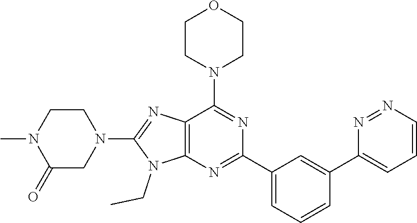 |
| 373 | 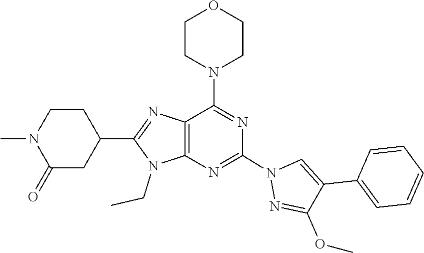 [SMILES: CCn1c(C2CCN(C)C(=O)C2)nc2c(N3CCOCC3)nc(-n3cc(-c4ccccc4)c(OC)n3)nc21] |
| 374 | 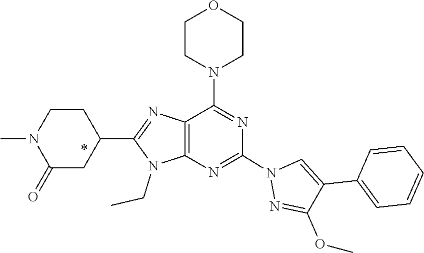 [SMILES: CCn1c(C2CCN(C)C(=O)C2)nc2c(N3CCOCC3)nc(-n3cc(-c4ccccc4)c(OC)n3)nc21] |
| 375 | 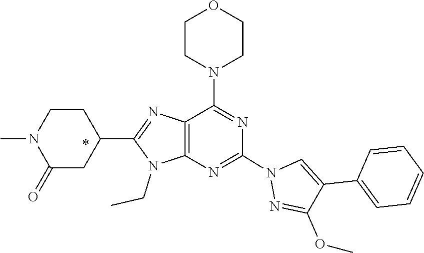 [SMILES: CCn1c(C2CCN(C)C(=O)C2)nc2c(N3CCOCC3)nc(-n3cc(-c4ccccc4)c(OC)n3)nc21] |
| 376 | 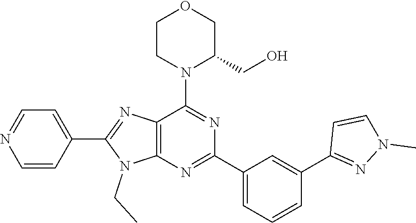 [SMILES: CCn1c(-c2ccncc2)nc2c(N3CCOC[C@@H]3CO)nc(-c3cccc(-c4ccn(C)n4)c3)nc21] |
| 377 | 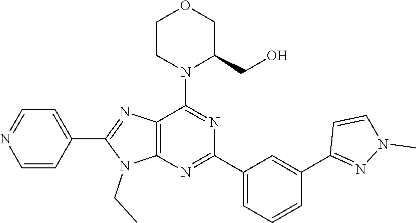 |
| 378 | 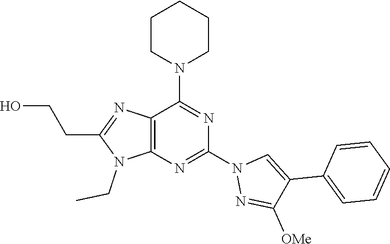 [SMILES: CCn1c(CCO)nc2c(N3CCCCC3)nc(-n3cc(-c4ccccc4)c(OC)n3)nc21] |
| 379 | 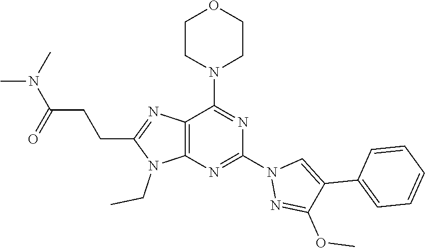 |
| 380 | 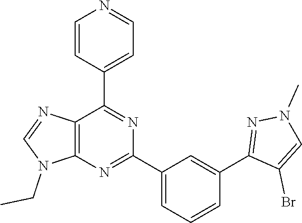 [SMILES: CCn1cnc2c(-c3ccncc3)nc(-c3cccc(-c4nn(C)cc4Br)c3)nc21] |
| 381 | 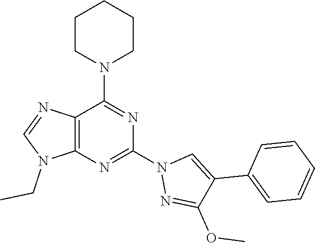 [SMILES: CCn1cnc2c(N3CCCCC3)nc(-n3cc(-c4ccccc4)c(OC)n3)nc21] |
| 382 | 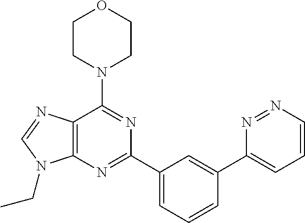 [SMILES: CCn1cnc2c(N3CCOCC3)nc(-c3cccc(-c4cccnn4)c3)nc21] |
| 383 | 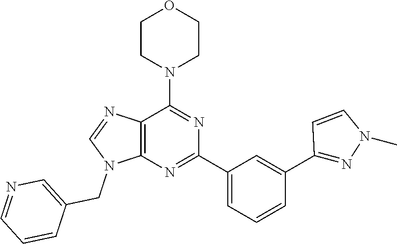 [SMILES: Cn1ccc(-c2cccc(-c3nc(N4CCOCC4)c4ncn(Cc5cccnc5)c4n3)c2)n1] |
| 384 | 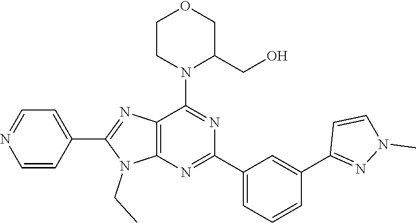 |
| 385 | 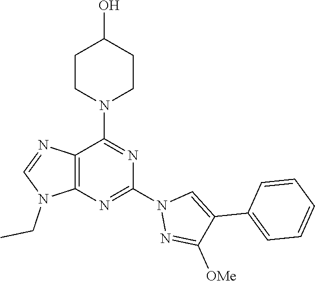 [SMILES: CCn1cnc2c(N3CCC(O)CC3)nc(-n3cc(-c4ccccc4)c(OC)n3)nc21] |
| 386 | 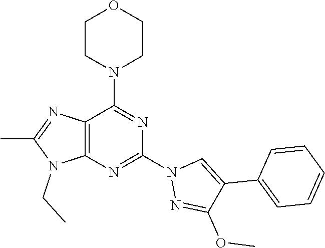 |
| 387 | 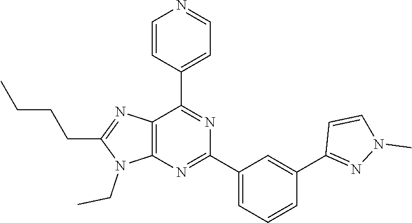 [SMILES: CCCCc1nc2c(-c3ccncc3)nc(-c3cccc(-c4ccn(C)n4)c3)nc2n1CC] |
| 388 | 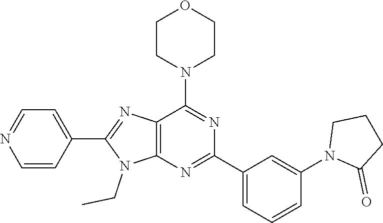 [SMILES: CCn1c(-c2ccncc2)nc2c(N3CCOCC3)nc(-c3cccc(N4CCCC4=O)c3)nc21] |
| 389 | 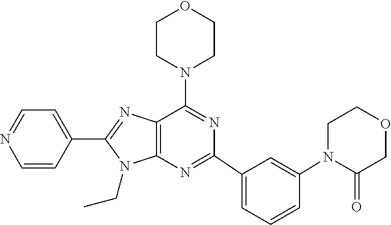 [SMILES: CCn1c(-c2ccncc2)nc2c(N3CCOCC3)nc(-c3cccc(N4CCOCC4=O)c3)nc21] |
| 390 | 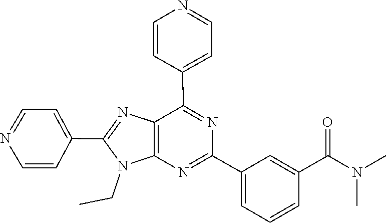 [SMILES: CCn1c(-c2ccncc2)nc2c(-c3ccncc3)nc(-c3cccc(C(=O)N(C)C)c3)nc21] |
| 391 | 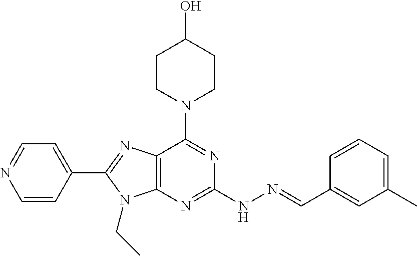 [SMILES: CCn1c(-c2ccncc2)nc2c(N3CCC(O)CC3)nc(N/N=C/c3cccc(C)c3)nc21] |
| 392 | 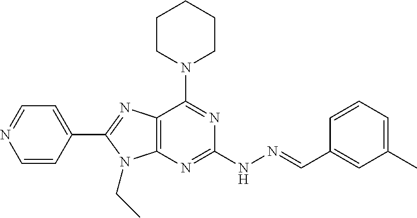 [SMILES: CCn1c(-c2ccncc2)nc2c(N3CCCCC3)nc(N/N=C/c3cccc(C)c3)nc21] |
| 393 | 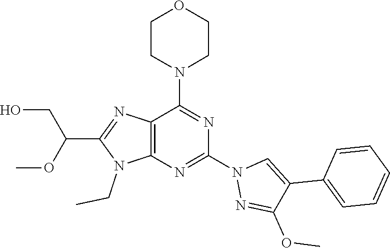 [SMILES: CCn1c(C(CO)OC)nc2c(N3CCOCC3)nc(-n3cc(-c4ccccc4)c(OC)n3)nc21] |
| 394 | 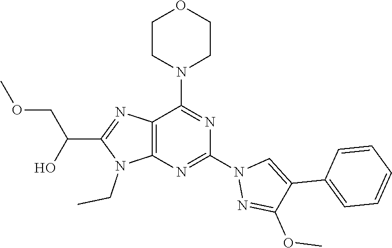 [SMILES: CCn1c(C(O)COC)nc2c(N3CCOCC3)nc(-n3cc(-c4ccccc4)c(OC)n3)nc21] |
| 395 | 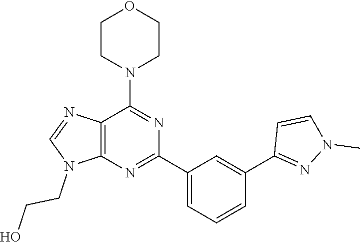 |
| 396 | 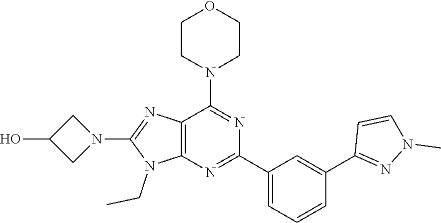 |
| 397 | 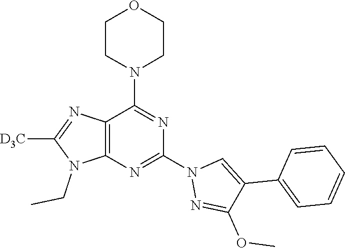 [SMILES: [2H]C([2H])([2H])c1nc2c(N3CCOCC3)nc(-n3cc(-c4ccccc4)c(OC)n3)nc2n1CC] |
| 398 | 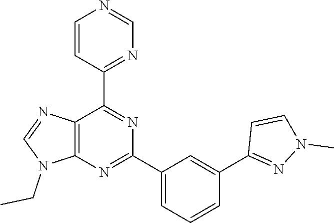 [SMILES: CCn1cnc2c(-c3ccncn3)nc(-c3cccc(-c4ccn(C)n4)c3)nc21] |
| 399 | 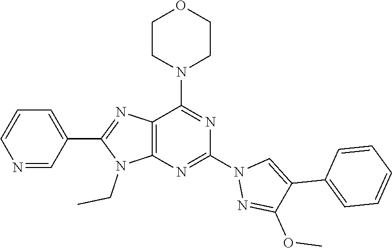 |
| 400 | 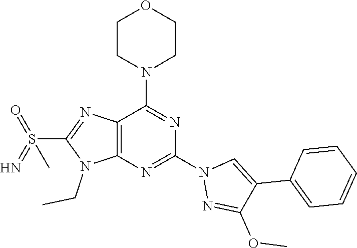 |
| 401 | 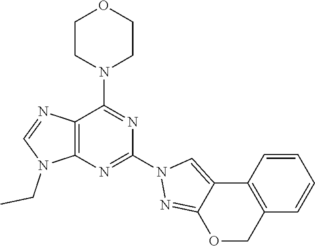 |
| 402 | 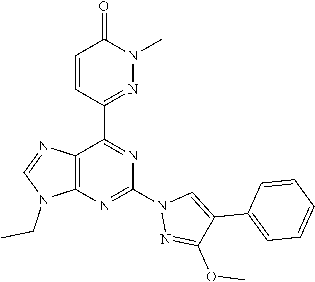 [SMILES: CCn1cnc2c(-c3ccc(=O)n(C)n3)nc(-n3cc(-c4ccccc4)c(OC)n3)nc21] |
| 403 | 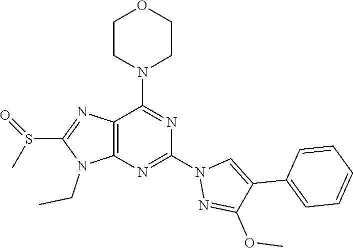 |
| 404 | 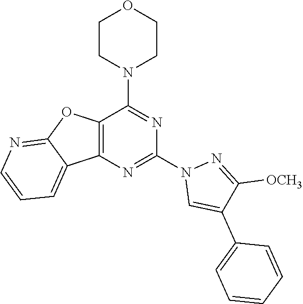 [SMILES: COc1nn(-c2nc(N3CCOCC3)c3oc4ncccc4c3n2)cc1-c1ccccc1] |
| 405 | 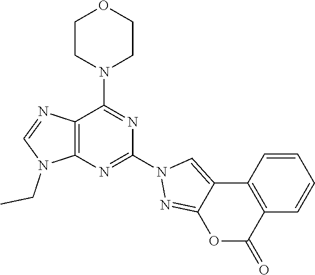 |
| 406 | 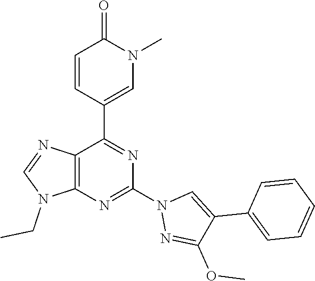 [SMILES: CCn1cnc2c(-c3ccc(=O)n(C)c3)nc(-n3cc(-c4ccccc4)c(OC)n3)nc21] |
| 407 | 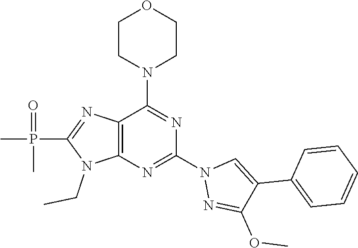 |
| 408 | 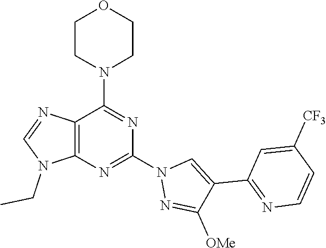 |
| 409 | 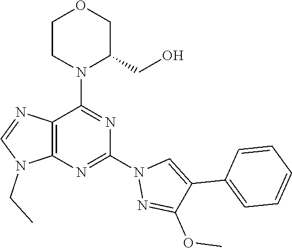 [SMILES: CCn1cnc2c(N3CCOC[C@@H]3CO)nc(-n3cc(-c4ccccc4)c(OC)n3)nc21] |
| 410 | 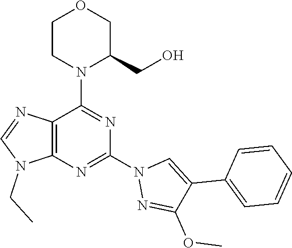 [SMILES: CCn1cnc2c(N3CCOC[C@H]3CO)nc(-n3cc(-c4ccccc4)c(OC)n3)nc21] |
| 411 | 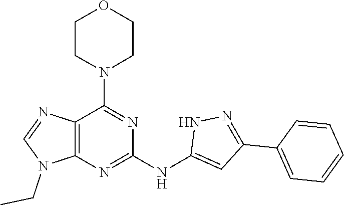 |
| 412 | 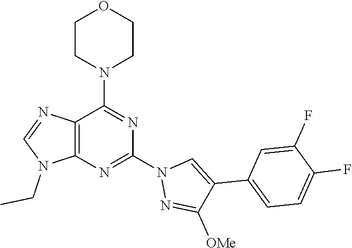 |
| 413 | 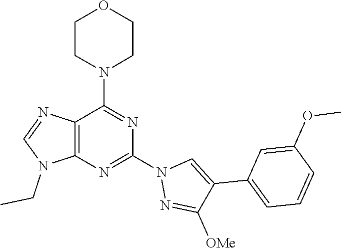 |
| 414 | 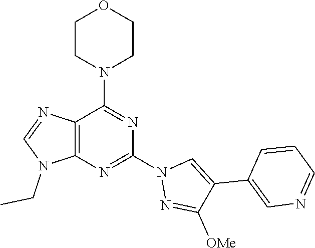 |
| 415 | 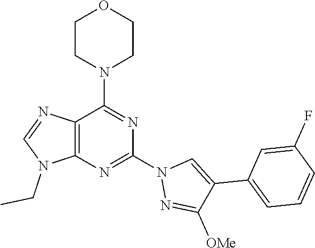 |
| 416 | 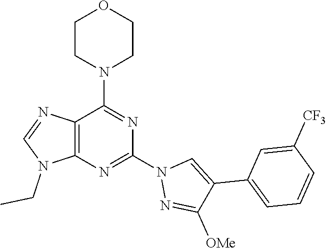 |
| 417 | 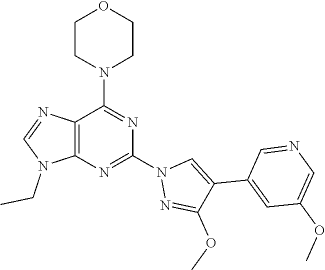 |
| 418 | 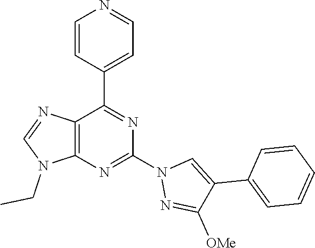 [SMILES: CCn1cnc2c(-c3ccncc3)nc(-n3cc(-c4ccccc4)c(OC)n3)nc21] |
| 419 | 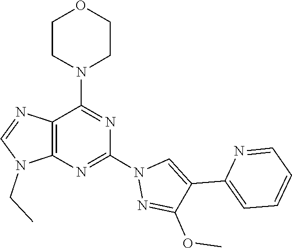 |
| 420 | 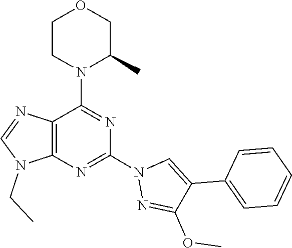 |
| 421 | 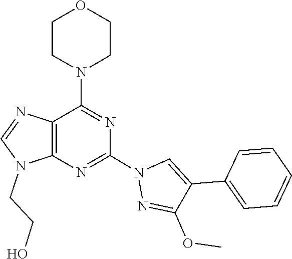 |
| 422 | 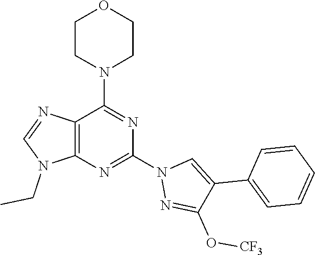 |
| 423 | 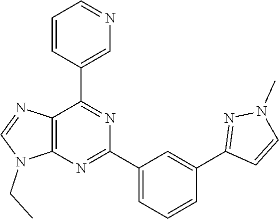 [SMILES: CCn1cnc2c(-c3cccnc3)nc(-c3cccc(-c4ccn(C)n4)c3)nc21] |
| 424 | 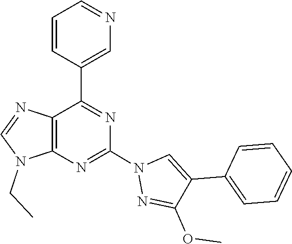 [SMILES: CCn1cnc2c(-c3cccnc3)nc(-n3cc(-c4ccccc4)c(OC)n3)nc21] |
| 425 | 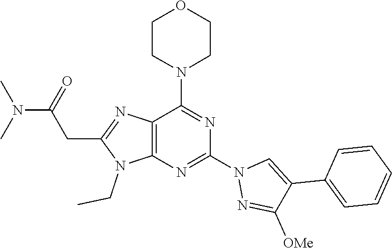 |
| 426 | 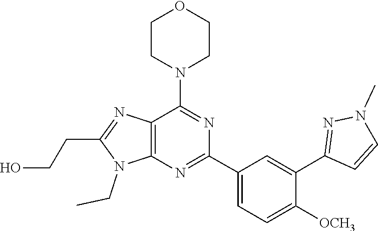 |
| 427 | 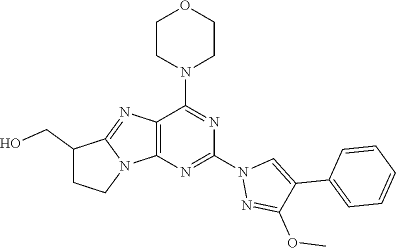 |
| 428 | 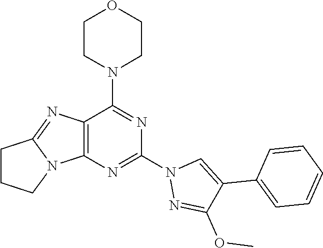 |
| 429 | 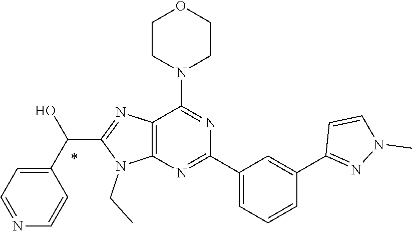 |
| 430 | 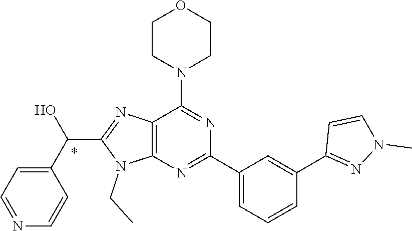 |
| 431 | 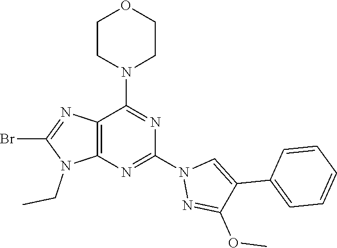 |
| 432 | 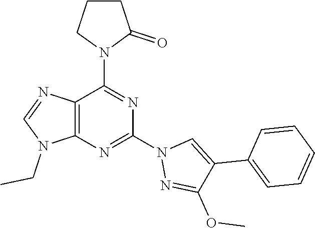 [SMILES: CCn1cnc2c(N3CCCC3=O)nc(-n3cc(-c4ccccc4)c(OC)n3)nc21] |
| 433 | 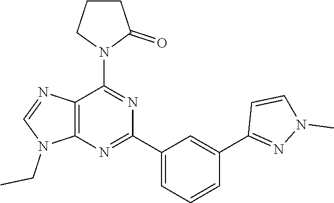 [SMILES: CCn1cnc2c(N3CCCC3=O)nc(-c3cccc(-c4ccn(C)n4)c3)nc21] |
| 434 | 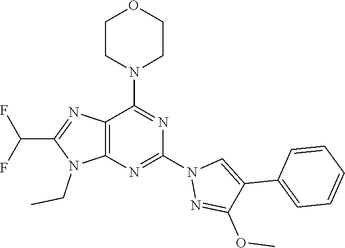 |
| 435 | 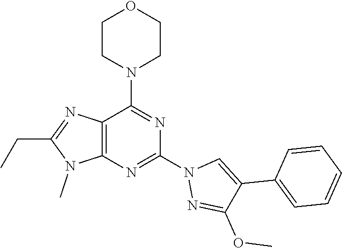 |
| 436 | 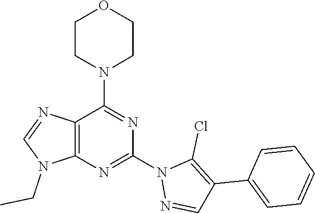 |
| 437 | 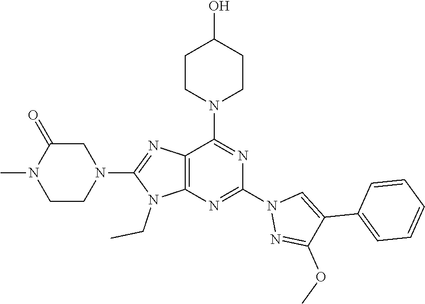 [SMILES: CCn1c(N2CCN(C)C(=O)C2)nc2c(N3CCC(O)CC3)nc(-n3cc(-c4ccccc4)c(OC)n3)nc21] |
| 438 | 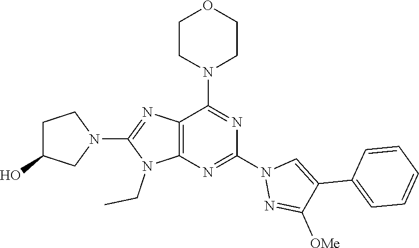 |
| 439 | 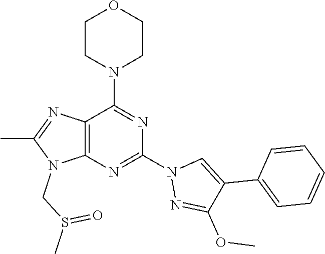 |
| 440 | 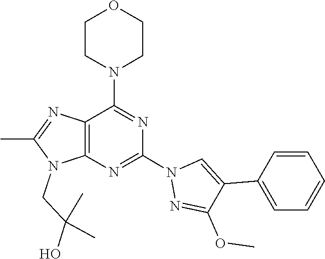 |
| 441 | 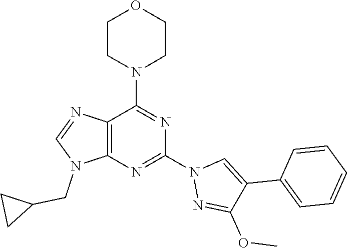 |
| 442 | 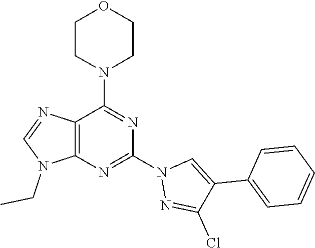 |
| 443 | 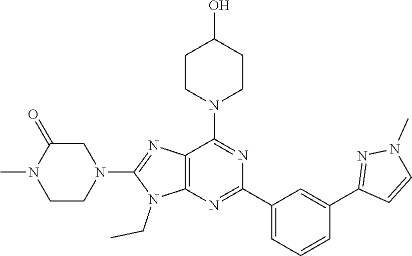 [SMILES: CCn1c(N2CCN(C)C(=O)C2)nc2c(N3CCC(O)CC3)nc(-c3cccc(-c4ccn(C)n4)c3)nc21] |
| 444 | 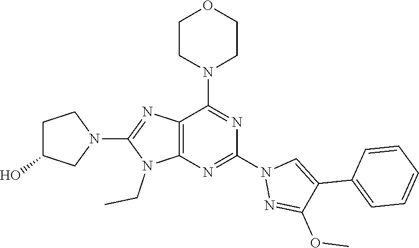 |
| 445 | 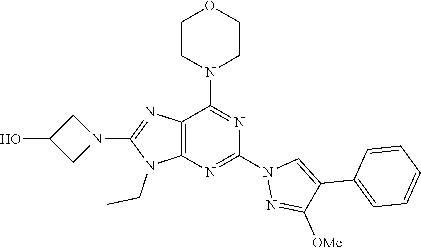 |
| 446 | 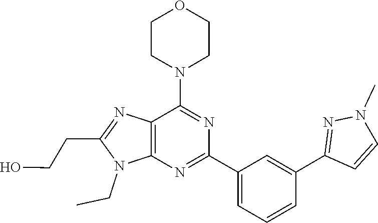 |
| 447 | 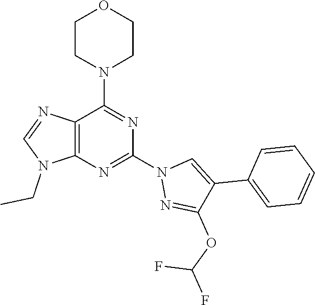 |
| 448 | 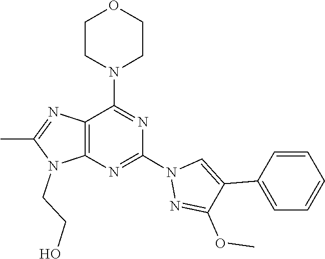 |
| 449 | 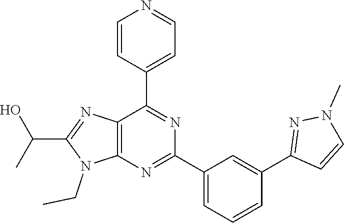 [SMILES: CCn1c(C(C)O)nc2c(-c3ccncc3)nc(-c3cccc(-c4ccn(C)n4)c3)nc21] |
| 450 | 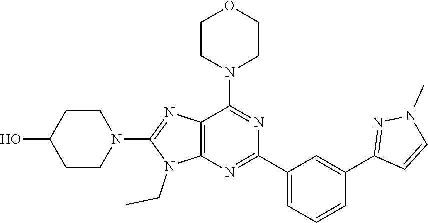 |
| 451 | 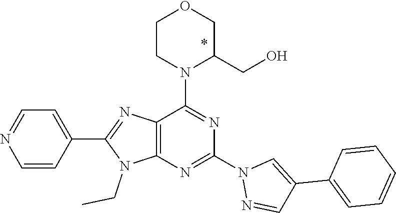 [SMILES: CCn1c(-c2ccncc2)nc2c(N3CCOCC3CO)nc(-n3cc(-c4ccccc4)cn3)nc21] |
| 452 | 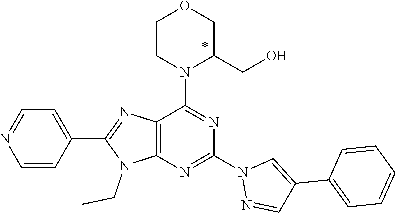 [SMILES: CCn1c(-c2ccncc2)nc2c(N3CCOCC3CO)nc(-n3cc(-c4ccccc4)cn3)nc21] |
| 453 | 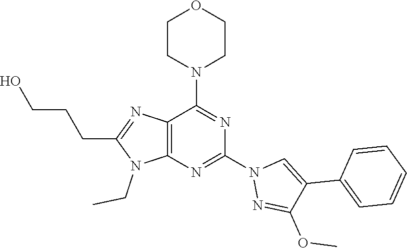 |
| 454 | 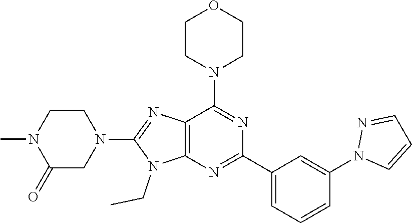 |
| 455 | 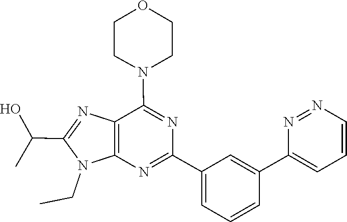 |
| 456 | 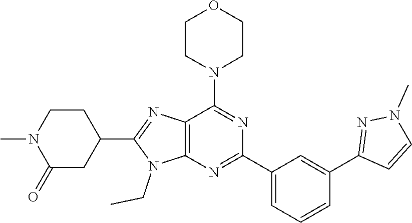 |
| 457 | 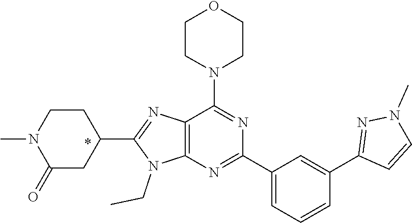 |
| 458 | 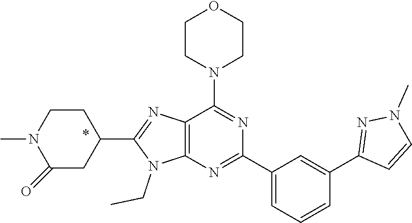 |
| 459 | 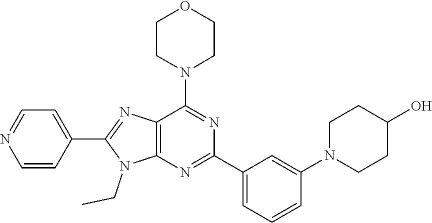 |
| 460 | 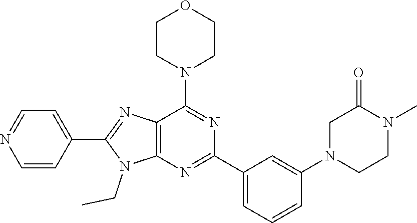 |
| 461 | 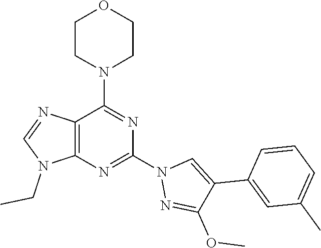 |
| 462 | 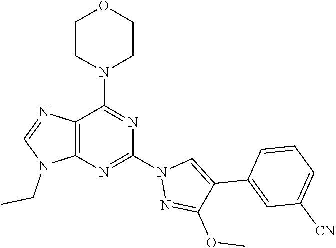 |
| 463 | 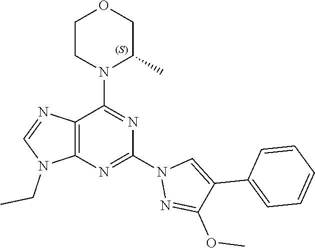 |
| 464 | 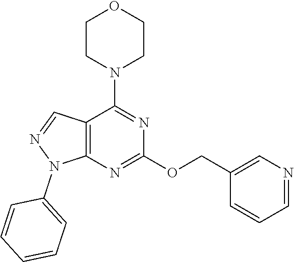 [SMILES: c1ccc(-n2ncc3c(N4CCOCC4)nc(OCc4cccnc4)nc32)cc1] |
| 465 | 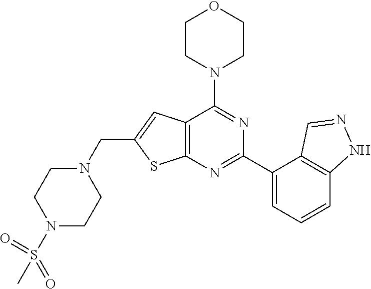 [SMILES: CS(=O)(=O)N1CCN(Cc2cc3c(N4CCOCC4)nc(-c4cccc5[nH]ncc45)nc3s2)CC1] |
| 466 | 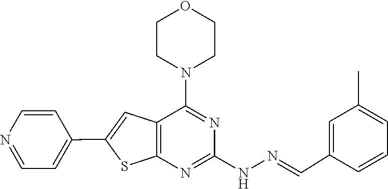 [SMILES: Cc1cccc(/C=N/Nc2nc(N3CCOCC3)c3cc(-c4ccncc4)sc3n2)c1] |
| 467 | 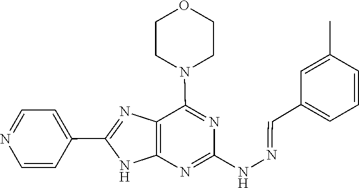 [SMILES: Cc1cccc(/C=N/Nc2nc(N3CCOCC3)c3nc(-c4ccncc4)[nH]c3n2)c1] |
| 468 | 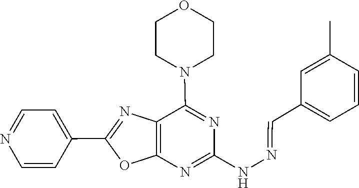 [SMILES: Cc1cccc(/C=N/Nc2nc(N3CCOCC3)c3nc(-c4ccncc4)oc3n2)c1] |
| 469 | 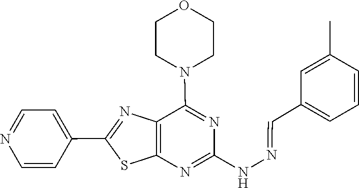 [SMILES: Cc1cccc(/C=N/Nc2nc(N3CCOCC3)c3nc(-c4ccncc4)sc3n2)c1] |
| 470 | 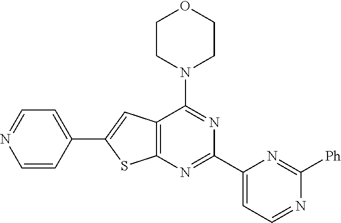 [SMILES: c1ccc(-c2nccc(-c3nc(N4CCOCC4)c4cc(-c5ccncc5)sc4n3)n2)cc1] |
| 471 | 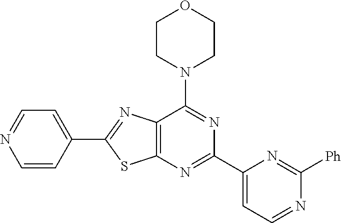 [SMILES: c1ccc(-c2nccc(-c3nc(N4CCOCC4)c4nc(-c5ccncc5)sc4n3)n2)cc1] |
| 472 | 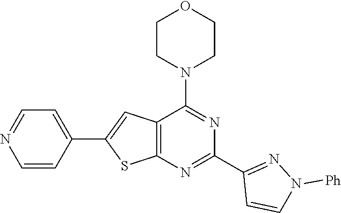 [SMILES: c1ccc(-n2ccc(-c3nc(N4CCOCC4)c4cc(-c5ccncc5)sc4n3)n2)cc1] |
| 473 | 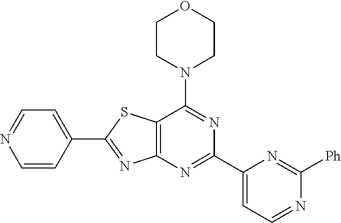 [SMILES: c1ccc(-c2nccc(-c3nc(N4CCOCC4)c4sc(-c5ccncc5)nc4n3)n2)cc1] |
| 474 | 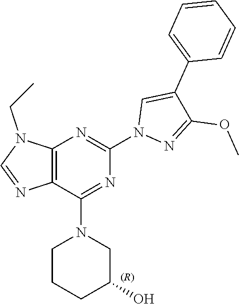 |
| 475 | 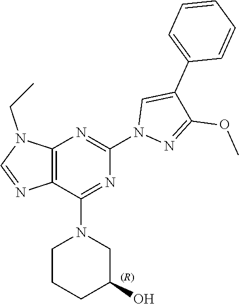 |
| 476 | 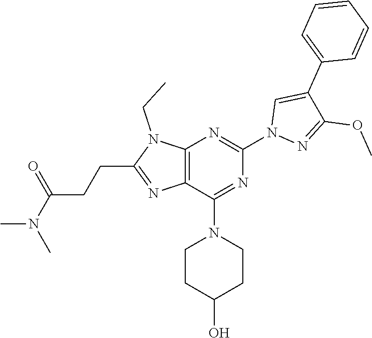 |

or a pharmaceutically acceptable salt thereof.

### Methods of Treatment

### Suppression of PIKfyve Activity and TDP-43 Aggregation to Treat Neurological Disorders

Using the compositions and methods described herein, a patient suffering from a neurological disorder may be administered a PIKfyve inhibitor, such as a small molecule described herein, so as to treat the disorder and/or to suppress one or more symptoms associated with the disorder. Exemplary neurological disorders that may be treated using the compositions and methods described herein are, without limitation, amyotrophic lateral sclerosis, frontotemporal degeneration, Alzheimer's disease, Parkinson's disease, dementia with Lewy Bodies, corticobasal degeneration, progressive supranuclear palsy, dementia parkinsonism ALS complex of Guam, Huntington's disease, IBMPFD, sporadic inclusion body myositis, myofibrillar myopathy, dementia pugilistica, chronic traumatic encephalopathy, Alexander disease, and hereditary inclusion body myopathy, as well as neuromuscular diseases such as congenital myasthenic syndrome, congenital myopathy, cramp fasciculation syndrome, Duchenne muscular dystrophy, glycogen storage disease type II, hereditary spastic paraplegia, inclusion body myositis, Isaac's Syndrome, Kearns-Sayre syndrome, Lambert-Eaton myasthenic syndrome, mitochondrial myopathy, muscular dystrophy, myasthenia gravis, myotonic dystrophy, peripheral neuropathy, spinal and bulbar muscular atrophy, spinal muscular atrophy, Stiff person syndrome, Troyer syndrome, and Guillain-Barre syndrome.

The present disclosure is based, in part, on the discovery that PIKfyve inhibitors, such as the agents described herein, are capable of attenuating TDP-43 toxicity. TDP-43-promoted toxicity has been associated with various neurological diseases. The discovery that PIKfyve inhibitors modulate TDP-43 aggregation provides an important therapeutic benefit. Using a PIKfyve inhibitor, such as a PIKfyve inhibitor described herein, a patient suffering from a neurological disorder or at risk of developing such a condition may be treated in a manner that remedies an underlying molecular etiology of the disease.

Without being limited by mechanism, the compositions and methods described herein can be used to treat or prevent such neurological conditions, for example, by suppressing the TDP-43 aggregation that promotes pathology.

Additionally, the compositions and methods described herein provide the beneficial feature of enabling the identification and treatment of patients that are likely to respond to PIKfyve inhibitor therapy. For example, in some embodiments, a patient (e.g., a human patient suffering from or at risk of developing a neurological disease described herein, such as amyotrophic lateral sclerosis) is administered a PIKfyve inhibitor if the patient is identified as likely to respond to this form of treatment. Patients may be identified as such on the basis, for example, of susceptibility to TDP-43 aggregation. In some embodiments, the patient is identified is likely to respond to PIKfyve inhibitor treatment based on the isoform of TDP-43 expressed by the patient. For example, patients expressing TDP-43 isoforms having a mutation selected from Q331K, M337V, Q343R, N345K, R361S, and N390D, among others, are more likely to develop TDP-43-promoted aggregation and toxicity relative to patients that do not express such isoforms of TDP-43. Using the compositions and methods described herein, a patient may be identified as likely to respond to PIKfyve inhibitor therapy on the basis of expressing such an isoform of TDP-43, and may subsequently be administered a PIKfyve inhibitor so as to treat or prevent one or more neurological disorders, such as one or more of the neurological disorders described herein.

### Assessing Patient Response

A variety of methods known in the art and described herein can be used to determine whether a patient having a neurological disorder (e.g., a patient at risk of developing TDP-43 aggregation, such as a patient expressing a mutant form of TDP-43 having a mutation associated with elevated TDP-43 aggregation and toxicity, for example, a mutation selected from Q331K, M337V, Q343R, N345K, R361S, and N390D) is responding favorably to PIKfyve inhibition. For example, successful treatment of a patient having a neurological disease, such as amyotrophic lateral sclerosis, with a PIKfyve inhibitor described herein may be signaled by:
(i) an improvement in condition as assessed using the amyotrophic lateral sclerosis functional rating scale (ALSFRS) or the revised ALSFRS (ALSFRS-R), such as an improvement in the patient's ALSFRS or ALSFRS-R score within one or more days, weeks, or months following administration of the PIKfyve inhibitor (e.g., an improvement in the patient's ALSFRS or ALSFRS-R score within from about 1 day to about 48 weeks (e.g., within from about 2 days to about 36 weeks, from about 4 weeks to about 24 weeks, from about 8 weeks to about 20 weeks, or from about 12 weeks to about 16 weeks), or more, following the initial administration of the PIKfyve inhibitor to the patient, such as within 1 day, 2 days, 3 days, 4 days, 5 days, 6 days, 7 days, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, or more, following the initial administration of the PIKfyve inhibitor to the patient);
(ii) an increase in slow vital capacity, such as an increase in the patient's slow vital capacity within one or more days, weeks, or months following administration of the PIKfyve inhibitor (e.g., an increase in the patient's slow vital capacity within from about 1 day to about 48 weeks (e.g., within from about 2 days to about 36 weeks, from about 4 weeks to about 24 weeks, from about 8 weeks to about 20 weeks, or from about 12 weeks to about 16 weeks), or more, following the initial administration of the PIKfyve inhibitor to the patient, such as within 1 day, 2 days, 3 days, 4 days, 5 days, 6 days, 7 days, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, or more, following the initial administration of the PIKfyve inhibitor to the patient); (iii) a reduction in decremental responses exhibited by the patient upon repetitive nerve stimulation, such as a reduction that is observed within one or more days, weeks, or months following administration of the PIKfyve inhibitor (e.g., a reduction that is observed within from about 1 day to about 48 weeks (e.g., within from about 2 days to about 36 weeks, from about 4 weeks to about 24 weeks, from about 8 weeks to about 20 weeks, or from about 12 weeks to about 16 weeks), or more, following the initial administration of the PIKfyve inhibitor to the patient, such as within 1 day, 2 days, 3 days, 4 days, 5 days, 6 days, 7 days, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, or more, following the initial administration of the PIKfyve inhibitor to the patient);
(iv) an improvement in muscle strength, as assessed, for example, by way of the Medical Research Council muscle testing scale (as described, e.g., in Jagtap et al., Ann. Indian. Acad. Neurol. 17:336-339 (2014), the disclosure of which is incorporated herein by reference as it pertains to measuring patient response to neurological disease treatment), such as an improvement that is observed within one or more days, weeks, or months following administration of the PIKfyve inhibitor (e.g., an improvement that is observed within from about 1 day to about 48 weeks (e.g., within from about 2 days to about 36 weeks, from about 4 weeks to about 24 weeks, from about 8 weeks to about 20 weeks, or from about 12 weeks to about 16 weeks), or more, following the initial administration of the PIKfyve inhibitor to the patient, such as within 1 day, 2 days, 3 days, 4 days, 5 days, 6 days, 7 days, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, or more, following the initial administration of the PIKfyve inhibitor to the patient);
(v) an improvement in quality of life, as assessed, for example, using the amyotrophic lateral sclerosis-specific quality of life (ALS-specific QOL) questionnaire, such as an improvement in the patient's quality of life that is observed within one or more days, weeks, or months following administration of the PIKfyve inhibitor (e.g., an improvement in the subject's quality of life that is observed within from about 1 day to about 48 weeks (e.g., within from about 2 days to about 36 weeks, from about 4 weeks to about 24 weeks, from about 8 weeks to about 20 weeks, or from about 12 weeks to about 16 weeks), or more, following the initial administration of the PIKfyve inhibitor to the patient, such as within 1 day, 2 days, 3 days, 4 days, 5 days, 6 days, 7 days, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, or more, following the initial administration of the PIKfyve inhibitor to the patient);
(vi) a decrease in the frequency and/or severity of muscle cramps, such as a decrease in cramp frequency and/or severity within one or more days, weeks, or months following administration of the PIKfyve inhibitor (e.g., a decrease in cramp frequency and/or severity within from about 1 day to about 48 weeks (e.g., within from about 2 days to about 36 weeks, from about 4 weeks to about 24 weeks, from about 8 weeks to about 20 weeks, or from about 12 weeks to about 16 weeks), or more, following the initial administration of the PIKfyve inhibitor to the patient, such as within 1 day, 2 days, 3 days, 4 days, 5 days, 6 days, 7 days, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, or more, following the initial administration of the PIKfyve inhibitor to the patient); and/or
(vii) a decrease in TDP-43 aggregation, such as a decrease in TDP-43 aggregation within one or more days, weeks, or months following administration of the PIKfyve inhibitor (e.g., a decrease in TDP-43 aggregation within from about 1 day to about 48 weeks (e.g., within from about 2 days to about 36 weeks, from about 4 weeks to about 24 weeks, from about 8 weeks to about 20 weeks, or from about 12 weeks to about 16 weeks), or more, following the initial administration of the PIKfyve inhibitor to the patient, such as within 1 day, 2 days, 3 days, 4 days, 5 days, 6 days, 7 days, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, or more, following the initial administration of the PIKfyve inhibitor to the patient.

### Combination Formulations and Uses Thereof

The compounds of the invention can be combined with one or more therapeutic agents. In particular, the therapeutic agent can be one that treats or prophylactically treats any neurological disorder described herein.

### Combination Therapies

A compound of the invention can be used alone or in combination with other agents that treat neurological disorders or symptoms associated therewith, or in combination with other types of treatment to treat, prevent, and/or reduce the risk of any neurological disorders. In combination treatments, the dosages of one or more of the therapeutic compounds may be reduced from standard dosages when administered alone. For example, doses may be determined empirically from drug combinations and permutations or may be deduced by isobolographic analysis (e.g., Black et al., Neurology 65:S3-S6, 2005). In this case, dosages of the compounds when combined should provide a therapeutic effect.

### Pharmaceutical Compositions

The compounds of the invention are preferably formulated into pharmaceutical compositions for administration to human subjects in a biologically compatible form suitable for administration in vivo. Accordingly, in another aspect, the present invention provides a pharmaceutical composition including a compound of the invention in admixture with a suitable diluent, carrier, or excipient.

The compounds of the invention may be used in the form of the free base, in the form of salts, solvates, and as prodrugs. All forms are within the scope of the invention. In accordance with the methods of the invention, the described compounds or salts, solvates, or prodrugs thereof may be administered to a patient in a variety of forms depending on the selected route of administration, as will be understood by those skilled in the art. The compounds of the invention may be administered, for example, by oral, parenteral, buccal, sublingual, nasal, rectal, patch, pump, or transdermal administration and the pharmaceutical compositions formulated accordingly. Parenteral administration includes intravenous, intraperitoneal, subcutaneous, intramuscular, transepithelial, nasal, intrapulmonary, intrathecal, rectal, and topical modes of administration. Parenteral administration may be by continuous infusion over a selected period of time.

A compound of the invention may be orally administered, for example, with an inert diluent or with an assimilable edible carrier, or it may be enclosed in hard or soft shell gelatin capsules, or it may be compressed into tablets, or it may be incorporated directly with the food of the diet. For oral therapeutic administration, a compound of the invention may be incorporated with an excipient and used in the form of ingestible tablets, buccal tablets, troches, capsules, elixirs, suspensions, syrups, and wafers.

A compound of the invention may also be administered parenterally. Solutions of a compound of the invention can be prepared in water suitably mixed with a surfactant. Dispersions can also be prepared in glycerol, liquid polyethylene glycols, DMSO and mixtures thereof with or without alcohol, and in oils. Under ordinary conditions of storage and use, these preparations may contain a preservative to prevent the growth of microorganisms. Conventional procedures and ingredients for the selection and preparation of suitable formulations are described, for example, in Remington's Pharmaceutical Sciences (2003, 20^{th} ed.) and in The United States Pharmacopeia: The National Formulary (USP 24 NF19), published in 1999.

The pharmaceutical forms suitable for injectable use include sterile aqueous solutions or dispersions and sterile powders for the extemporaneous preparation of sterile injectable solutions or dispersions. In all cases the form must be sterile and must be fluid to the extent that may be easily administered via syringe.

Compositions for nasal administration may conveniently be formulated as aerosols, drops, gels, and powders. Aerosol formulations typically include a solution or fine suspension of the active substance in a physiologically acceptable aqueous or non-aqueous solvent and are usually presented in single or multidose quantities in sterile form in a sealed container, which can take the form of a cartridge or refill for use with an atomizing device. Alternatively, the sealed container may be a unitary dispensing device, such as a single dose nasal inhaler or an aerosol dispenser fitted with a metering valve which is intended for disposal after use. Where the dosage form includes an aerosol dispenser, it will contain a propellant, which can be a compressed gas, such as compressed air or an organic propellant, such as fluorochlorohydrocarbon. The aerosol dosage forms can also take the form of a pump-atomizer. Compositions suitable for buccal or sublingual administration include tablets, lozenges, and pastilles, where the active ingredient is formulated with a carrier, such as sugar, acacia, tragacanth, gelatin, and glycerine. Compositions for rectal administration are conveniently in the form of suppositories containing a conventional suppository base, such as cocoa butter.

The compounds of the invention may be administered to an animal, e.g., a human, alone or in combination with pharmaceutically acceptable carriers, as noted herein, the proportion of which is determined by the solubility and chemical nature of the compound, chosen route of administration, and standard pharmaceutical practice.

### Dosages

The dosage of the compounds of the invention, and/or compositions including a compound of the invention, can vary depending on many factors, such as the pharmacodynamic properties of the compound; the mode of administration; the age, health, and weight of the recipient; the nature and extent of the symptoms; the frequency of the treatment, and the type of concurrent treatment, if any; and the clearance rate of the compound in the animal to be treated. One of skill in the art can determine the appropriate dosage based on the above factors. The compounds of the invention may be administered initially in a suitable dosage that may be adjusted as required, depending on the clinical response. In general, satisfactory results may be obtained when the compounds of the invention are administered to a human at a daily dosage of, for example, between 0.05 mg and 3000 mg (measured as the solid form). Dose ranges include, for example, between 10-1000 mg.

Alternatively, the dosage amount can be calculated using the body weight of the patient. For example, the dose of a compound, or pharmaceutical composition thereof, administered to a patient may range from 0.1-50 mg/kg.

The following examples are meant to illustrate the invention. They are not meant to limit the invention in any way.

### EXAMPLES

List of Abbreviations:

| | |
|---|---|
| ADDP | 1,1′-(Azodicarbonyl)dipiperidine |
| BAST | Bis(2-methoxyethyl)aminosulfur trifluoride |
| Boc2O | Di-tert-butyl dicarbonate |
| BPO | Benzoyl peroxide |
| CAN | Ceric ammonium nitrate |
| CO | Carbon monoxide |
| DCE | 1,2-Dichloroethane |
| DCM | Dichloromethane |
| DHP | Dihydropyran |
| DIPEA | N,N-Diisopropylethylamine |
| DMA | N,N-Dimethylaniline |
| DMAc | Dimethylacetamide |
| DMAP | 4-Dimethylaminopyridine |
| DMF | N,N-Dimethylformamide |
| DMP | Des-Martin periodinane |
| DMS | Dimethylsulfate |
| DMSO | Dimethylsulfoxide |
| DPPA | Diphenylphosphoryl azide |
| DPPF | 1,1′-bis(diphenylphosphino)ferrocene |
| EA | Ethyl acetate |
| EDCI | 1-Ethyl-3-(3-dimethylaminopropyl)carbodiimide |
| For NMR | S-singlet, d-doublet, dd-doublet-of-doublet, dt-doublet of |
| | triplet, q-quartet, bs-broad singlet, dpent, doublet of |
| | pentet, t-triplet, pent-pentet |
| h | Hour(s) |
| HATU | Hexafluorophosphate Azabenzotriazole Tetramethyl |
| | Uronium |
| HMBC | Heteronuclear Multiple Bond Correlation |
| JohnPhos | [1,1′-biphenyl]-2-yldi-tert-butylphosphane |
| LAH | Lithium aluminum hydride |
| LDA | Lithium diisopropylamide |
| LiHMDS | Lithium 1,1,1-trimethyl-N-(trimethylsilyl)silanaminide |
| mW | Microwave |
| NaHMDS | Sodium hexamethyldisilazide |
| NBS | N-bromosuccinimide |
| NMO | N-Methylmorpholine N-oxide |
| NMP | N-Methylpyrrolidone |
| o/n | Overnight |
| Pbu3 | Tributylphosphine |
| Pcy3 | Tricyclohexylphosphine |
| Pd(tBu3P)2 | Bis(tri-tert-butylphosphine)palladium(0) |
| Pd2(dba)3 | Tris(dibenzylideneacetone)dipalladium(0) |
| PdCl2(dppf) | [1,1′- |
| | Bis(diphenylphosphino)ferrocene]dichloropalladium(II) |
| Py | Pyridine |
| PyBOP | Benzotriazol-1-yloxytripyrrolidinophosphonium |
| | hexafluorophosphate |
| Qphos | 1,2,3,4,5-Pentaphenyl-1′-(di-tert-butylphosphino)ferrocene |
| RT | Room temperature |
| Rt. | Retention Time |
| SEM | 2-(Trimethylsilyl)ethoxymethyl |
| TBAC | Tetrabutylammonium chloride |
| TBAF | Tetra-n-butylammonium fluoride |
| TEA | Triethylamine |
| TES | Triethylsilane |
| TFA | Trifluoroacetic acid |
| THF | Tetrahydrafuran |
| TMEDA | Tetramethylethylenediamine |
| TMS-CL | Trimethylsilyl chloride |
| TsOH | p-Toluenesulfonic acid |
| Xantphos | 4,5-Bis(diphenylphosphino)-9,9-dimethylxanthene |
| Xphos | 2-Dicyclohexylphosphino-2′,4′,6′-triisopropylbiphenyl |

### Example 1. Preparation of Compounds

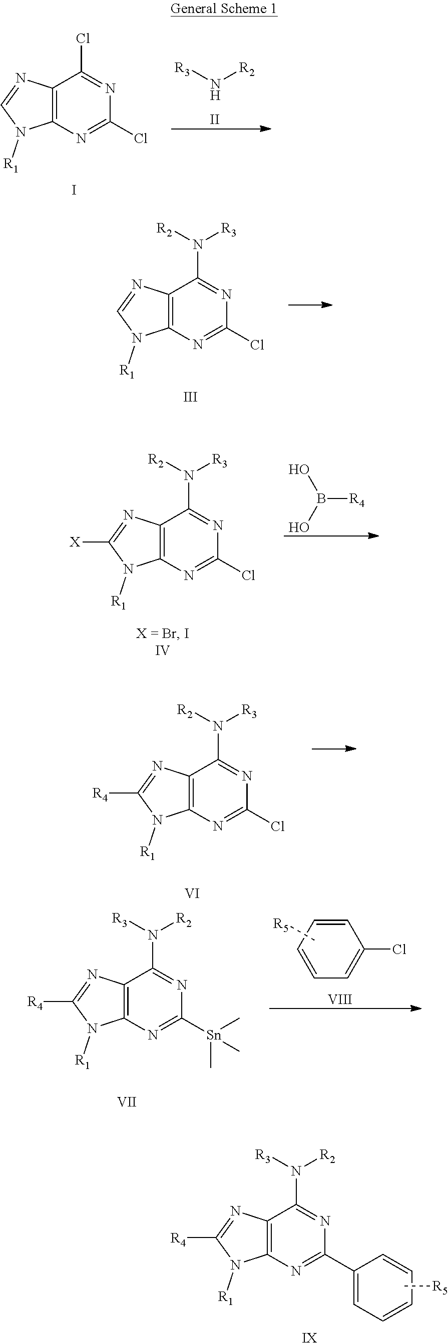

An appropriately substituted aryl chloride I is reacted with an appropriately substituted amine II under basic conditions (e.g., N,N-diisopropylethylamine) to afford appropriately substituted aryl chloride III. Aryl chloride III is halogenated with a bromine or iodide source (e.g., N-bromosuccinimide) to afford appropriately substituted aryl halide IV. Aryl halide IV is reacted with appropriately substituted boronic acid V in the presence of a palladium source (e.g., 1,1′-Bis(diphenylphosphino)ferrocene dichloropalladium(II)) to afford appropriately substituted aryl chloride VI. Aryl chloride VI is coupled with 1,1,1,2,2,2-hexamethyldistannane in the presence of a palladium source (e.g., bis(triphenylphosphine)palladium(II) dichloride) to afford appropriately substituted organostannane VII. Organostannane VII is coupled with appropriately substituted aryl chloride VIII in the presence of a palladium source (e.g., tetrakis(triphenylphosphine)palladium(0)) to afford desired purine IX.

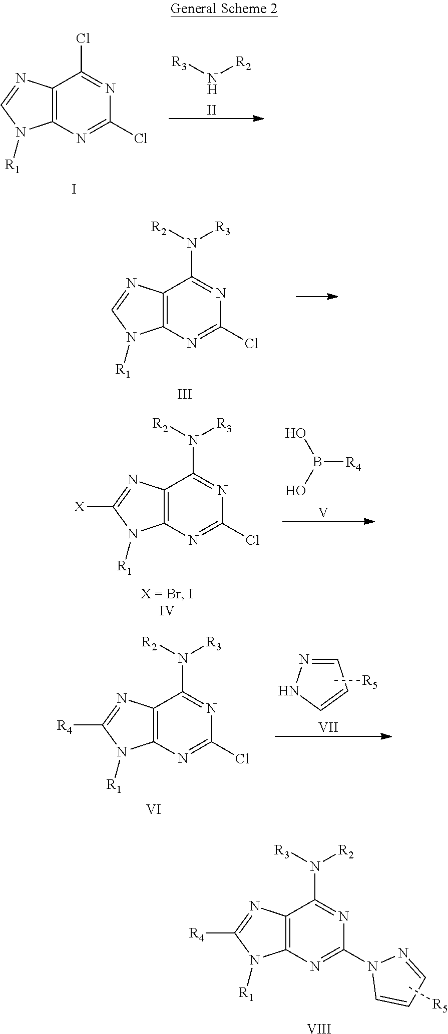

An appropriately substituted aryl chloride I is reacted with an appropriately substituted amine II under basic conditions (e.g., triethylamine) to afford appropriately substituted aryl chloride III. Aryl chloride III is halogenated with a bromine or iodide source (e.g., N-bromosuccinimide) to afford appropriately substituted aryl halide IV. Aryl halide IV is reacted with appropriately substituted boronic acid V in the presence of a palladium source (e.g., 1,1′-bis(diphenylphosphino)ferrocene dichloropalladium(II)) to afford appropriately substituted aryl chloride VI. Aryl chloride VI is coupled with appropriately substituted pyrazole VII under basic conditions (e.g., cesium carbonate) to afford desired purine VIII.

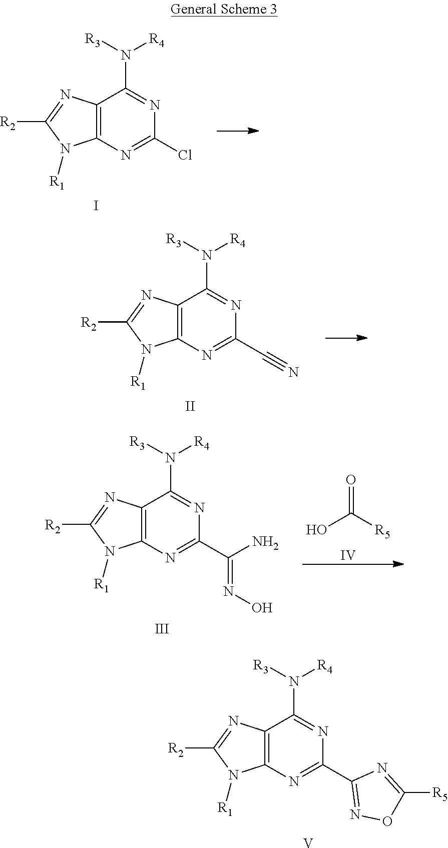

An appropriately substituted aryl chloride I is coupled with zinc cyanide in the presence of a palladium source (e.g., tetrakis(triphenylphosphine)palladium(0)) to afford appropriately substituted aryl nitrile II. Aryl nitrile II is coupled with hydroxylamine to afford appropriately substituted oxime III. Oxime III is reacted with appropriately substituted carboxylic acid IV in the presence of a coupling agent (e.g., HATU) to afford desired purine V.

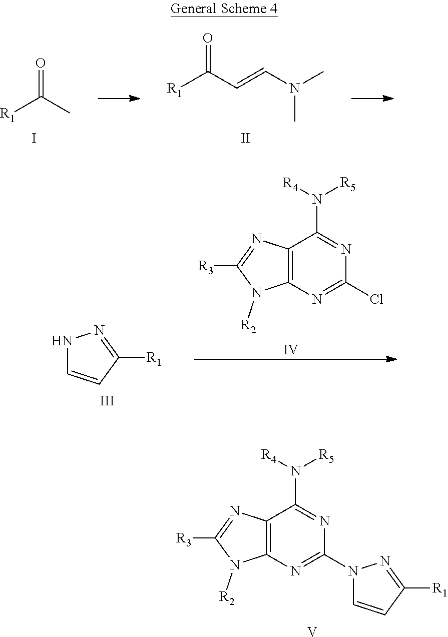

An appropriately substituted methyl ketone I is coupled N,N-dimethylformamide dimethyl acetal with heat to afford appropriately substituted enone II. Enone II is condensed with hydrazine monohydrate to afford appropriately substituted pyrazole Ill. Pyrazole III is reacted with appropriately substituted aryl chloride IV under basic conditions (e.g., cesium carbonate) and/or in the presence of a palladium source (e.g., tris(dibenzylideneacetone) dipalladium) to afford desired purine V.

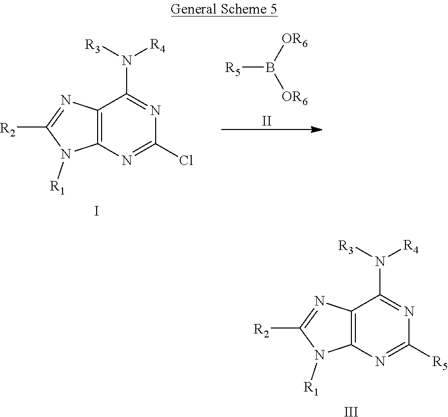

An appropriately substituted aryl chloride I is reacted with appropriately substituted boronic acid or ester II in the presence of a palladium catalyst (e.g., 1,1′-Bis(diphenylphosphino)ferrocene palladium(II)dichloride) to afford desired purine Ill.

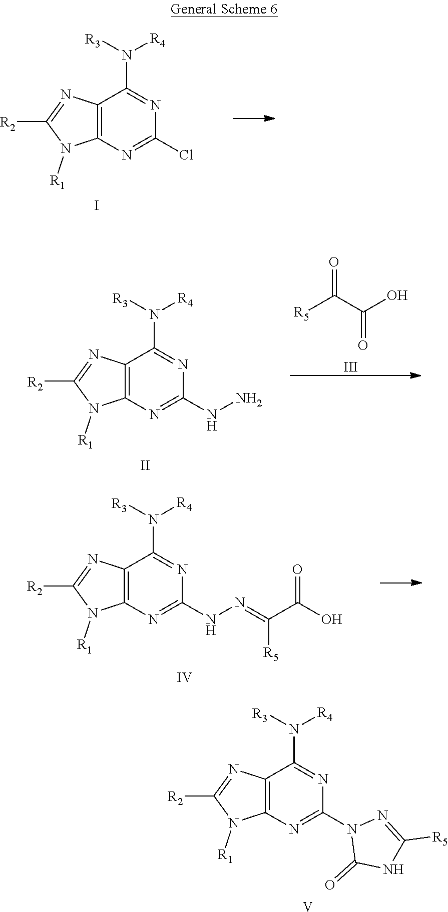

An appropriately substituted aryl chloride I is reacted with hydrazine hydrate with heat to afford appropriately substituted hydrazine II. Hydrazine II is reacted with appropriately substituted alpha-keto acid III under acidic conditions (e.g., hydrochloric acid) to afford appropriately substituted hydrazone IV. Hydrazone IV is condensed with diphenyl phosphorylazide under basic conditions (e.g., triethylamine) to afford desired purine V.

### Synthesis of 4-(9-methyl-8-(pyridin-4-yl)-9H-purin-6-yl)morpholine (Compound 1)

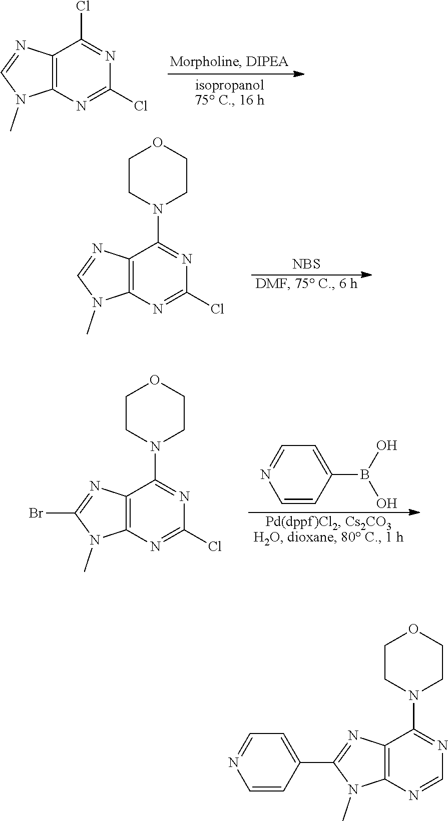

### Step 1: Synthesis of 4-(2-chloro-9-methyl-9H-purin-6-yl)morpholine

A solution of 2,6-dichloro-9-methyl-9H-purine (2 g, 9.85 mmol), morpholine (0.86 g, 9.85 mmol) and N,N-diisopropylethylamine (2.54 g, 19.7 mmol) in isopropanol (80 mL) was stirred at 75° C. for 16 h. The mixture was filtered to obtain 4-(2-chloro-9-methyl-9H-purin-6-yl)morpholine (2 g, 80%) as white solid. LCMS (ESI) m/z: 254.1 [M+H]⁺.

### Step 2: Synthesis of 4-(8-bromo-2-chloro-9-methyl-9H-purin-6-yl)morpholine

A solution of 4-(2-chloro-9-methyl-9H-purin-6-yl)morpholine (2 g, 7.88 mmol) and N-bromosuccinimide

(2.1 g, 11.82 mmol) in DMF (40 mL) was stirred at 75° C. for 6 h. The mixture was cooled to 20° C. and filtered. The solid was washed with ethyl acetate to obtain 4-(8-bromo-2-chloro-9-methyl-9H-purin-6-yl)morpholine (0.75 g, 29%) as white solid. LCMS (ESI) m/z: 332.0/334.0 [M+H]⁺.

### Step 3: Synthesis of 4-(9-methyl-8-(pyridin-4-yl)-9H-purin-6-yl)morpholine

A solution of 4-(8-bromo-2-chloro-9-methyl-9H-purin-6-yl)morpholine (0.75 g, 2.25 mmol), pyridin-4-ylboronic acid (0.28 g, 2.25 mmol), [1,1′-Bis(diphenylphosphino)ferrocene]dichloropalladium(II) (0.17 g, 0.23 mmol) and cesium carbonate (1.47 g, 4.5 mmol) in water (2 mL) and dioxane (10 mL) was stirred at 80° C. for 1 h under Argon. The mixture was diluted with ethyl acetate (50 mL) and washed with water (50 mL). The organic layer was concentrated and purified by prep-HPLC (Boston C18 21*250 mm 10 μm column. The mobile phase was acetonitrile/0.01% aqueous trifluoroacetic acid.) to obtain 4-(9-methyl-8-(pyridin-4-yl)-9H-purin-6-yl)morpholine (0.03 g, 5%) as white solid.

¹H NMR (400 MHz, DMSO-d₆) δ 8.71 (d, J=5.9 Hz, 2H), 8.32-8.23 (m, 3H), 4.32 (s, 4H), 3.83 (s, 3H), 3.77 (t, J=4.8 Hz, 4H). LCMS (ESI) m/z: 297.1 [M+H]⁺.

### Preparation of 7-methyl-6-(morpholin-4-yl)-2-(2-phenylpyrimidin-4-yl)-8-(pyridin-4-yl)-7H-purine (Compound 2)

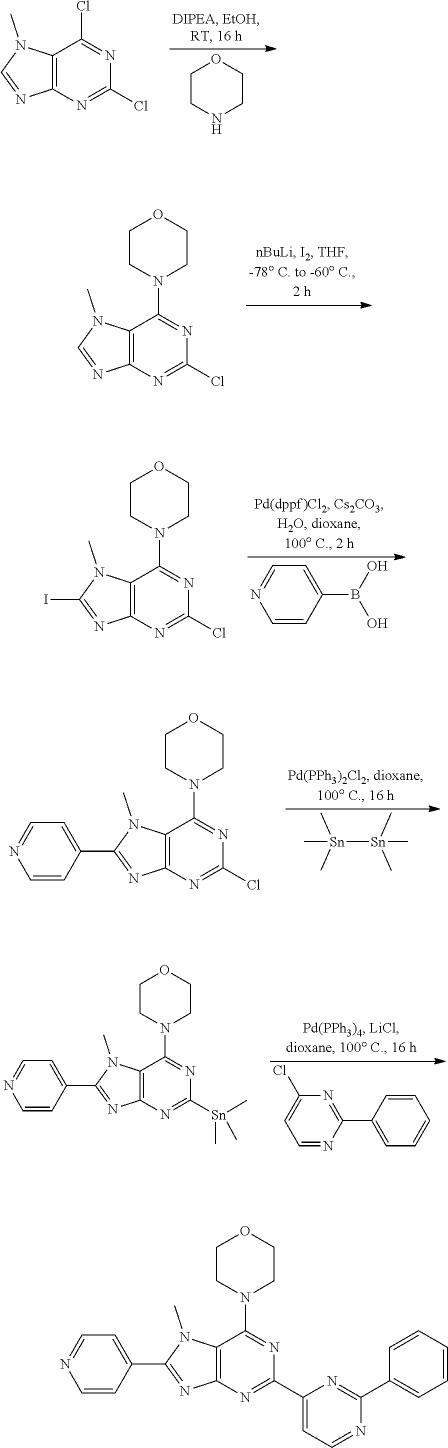

### Step 1: Preparation of 4-(2-chloro-7-methyl-7H-purin-6-yl)morpholine

To a solution of 2,6-dichloro-7-methyl-7H-purine (4.80 g, 24 mmol), morpholine (2.27 g, 26 mmol) in ethanol (100 mL) was added DIPEA (3.06 g, 24 mmol) and the reaction mixture was stirred at room temperature for 16 h. The precipitate formed was collected by filtration, washed with ethanol, and dried under vacuum to afford 4-(2-chloro-7-methyl-7H-purin-6-yl)morpholine (5.00 g, 20 mmol, 83%) as a white solid. ¹H NMR (500 MHz, Chloroform-d) δ 7.97 (s, 1H), 4.01 (s, 3H), 3.93-3.83 (m, 4H), 3.58-3.48 (m, 4H); LCMS (ESI) m/z: 254.1 [M+H]⁺.

### Step 2: Preparation of 4-(2-chloro-8-iodo-7-methyl-7H-purin-6-yl)morpholine

To a solution of 4-(2-chloro-7-methyl-7H-purin-6-yl)morpholine (4.50 g, 18 mmol) in tetrahydrofuran (270 ml) was added a 2.5 M solution of n-butyllithium in hexanes (8.5 mL, 21 mmol) at −78° C. and the resultant mixture was stirred at −78° C. for 30 minutes. A solution of iodine (6.75 g, 27 mmol) in tetrahydrofuran (30 mL) was then added to the reaction mixture and it was allowed to warm to −60° C. over 2 h with stirring. A solution of saturated sodium thiosulfate (200 mL) was added to the reaction vial at −60° C. and then the mixture was extracted with ethyl acetate (2×500 mL). The organic layers were pooled, washed with brine (100 mL), dried over anhydrous sodium sulfate, filtered and concentrated under reduced pressure. The crude residue was purified via flash column chromatography through silica gel using a gradient of 0-5% methanol in dichloromethane to obtain 4-(2-chloro-8-iodo-7-methyl-7H-purin-6-yl)morpholine (2.30 g, 6.1 mmol, 34%) as a yellow solid. LCMS (ESI) m/z: 216.1 [M+H]⁺.

### Step 3: Preparation of 4-(2-chloro-7-methyl-8-(pyridin-4-yl)-7H-purin-6-yl)morpholine

To a solution of 4-(2-chloro-8-iodo-7-methyl-7H-purin-6-yl)morpholine (2.30 g, 6.1 mmol) in dioxane (120 mL) and water (30 mL) was added pyridin-4-ylboronic acid (0.372 g, 3.0 mmol), cesium carbonate (0.197 g, 0.61 mmol) and [1,1′Bis(diphenylphosphino)ferrocene]dichloropalladium(II) (0.219 g, 0.30 mmol) and the mixture was stirred at 100° C. under argon for 2 h. Water (500 mL) was added to the reaction mixture and the mixture was extracted with ethyl acetate (3×500 mL). The organic layers were pooled, dried over anhydrous sodium sulfate, filtered and concentrated under reduced pressure. The crude residue was purified via flash column chromatography through silica gel using a gradient of 0-10% methanol in dichloromethane. The product 4-(2-chloro-7-methyl-8-(pyridin-4-yl)-7H-purin-6-yl)morpholine (0.750 g, 75%) was obtained as a yellow solid. LCMS (ESI) m/z: 331.0 [M+H]⁺.

### Step 4: Preparation of 4-(7-methyl-8-(pyridin-4-yl)-2-(trimethylstannyl)-7H-purin-6-yl)morpholine

To a solution of 4-(2-chloro-7-methyl-8-(pyridin-4-yl)-7H-purin-6-yl)morpholine (281 mg, 0.85 mmol) in dioxane (10 mL) were added 1,1,1,2,2,2-hexamethyldistannane (557 mg, 1.7 mmol) and bis(triphenylphosphine)palladium(II) dichloride (91.0 mg, 0.13 mmol). The reaction mixture was stirred at 100° C. for 2 h, allowed to cool to room temperature and then a 4 M solution of aqueous potassium fluoride (50 mL) was added. The resultant reaction mixture was stirred for 30 minutes and filtered over celite. The filtrate was extracted with dichloromethane (2×60 mL), washed with brine (40 mL), dried over sodium sulfate, and concentrated under reduced pressure. The crude product 4-(7-methyl-8-(pyridin-4-yl)-2-(trimethylstannyl)-7H-purin-6-yl)morpholine (390 mg, 0.85 mmol, 100%) was obtained as a brown solid and carried onto next step without further purification. LCMS (ESI) m/z: 459.0 [M+H]⁺.

### Step 5: Preparation of 4-(7-methyl-2-(2-phenylpyrimidin-4-yl)-8-(pyridin-4-yl)-7H-purin-6-yl)morpholine

To a mixture of 4-(7-methyl-8-(pyridin-4-yl)-2-(trimethylstannyl)-7H-purin-6-yl)morpholine (390 mg, 0.85 mmol), 4-chloro-2-phenylpyrimidine (194 mg, 1.0 mmol), and lithium chloride (89.0 mg, 2.13 mmol) in dioxane (10 mL) was added tetrakis(triphenylphosphine)palladium(0) (98.0 mg, 0.085 mmol). The reaction mixture was stirred at 100° C. for 16 h under argon. The reaction mixture was allowed to cool to room temperature, then filtered over celite and washed with ethyl acetate (2×30 mL). The filtrate was concentrated under reduced pressure. The crude product was purified by prep-HPLC (the crude samples were dissolved in N,N-dimethylformamide unless otherwise noted before purification. Boston pHlex ODS 10 um 21.2×250 mm 120 A. The mobile phase was acetonitrile/0.1% Ammonium bicarbonate) to give product 4-(7-methyl-2-(2-phenylpyrimidin-4-yl)-8-(pyridin-4-yl)-7H-purin-6-yl)morpholine (14.1 mg, 0.031 mmol, 3.3%) as a white solid.

¹H NMR (500 MHz, Chloroform-d) δ 8.98 (d, J=5.1 Hz, 1H), 8.88 (d, J=5.1 Hz, 2H), 8.69-8.62 (m, 2H), 8.37 (d, J=5.1 Hz, 1H), 7.85 (d, J=5.2 Hz, 2H), 7.56-7.48 (m, 3H), 4.12 (s, 3H), 4.05-3.98 (m, 4H), 3.75 (t, J=4.6 Hz, 4H). LCMS (ESI) m/z: 451.0 [M+H]⁺.

### Synthesis of 4-(2-(2-(furan-3-yl)pyrimidin-4-yl)-9-methyl-8-(pyridin-4-yl)-9H-purin-6-yl)morpholine (Compound 3)

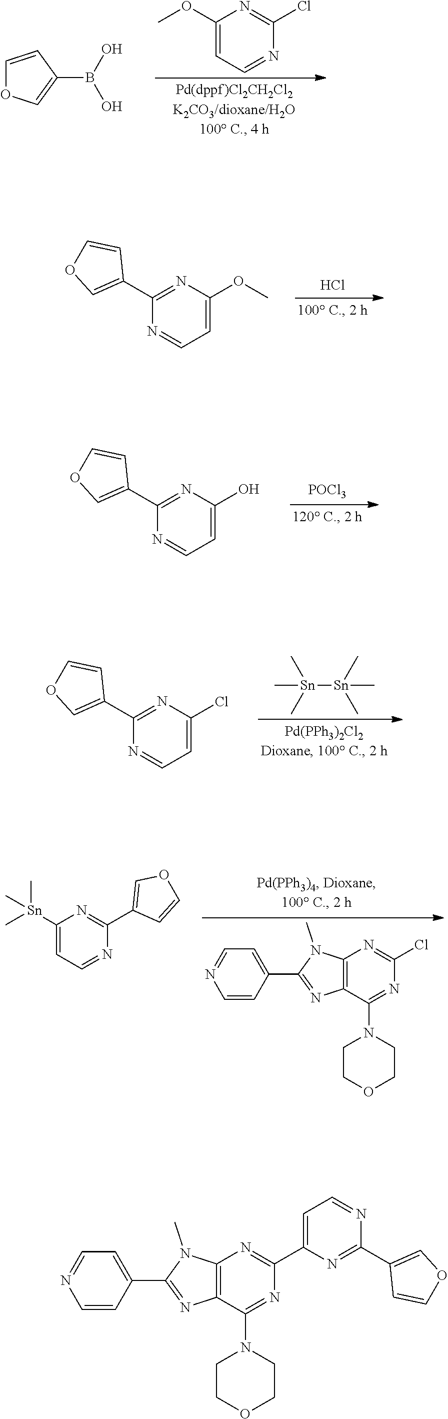

### Step 1: Synthesis of 2-(furan-3-yl)-4-methoxypyrimidine

To a solution of furan-3-ylboronic acid (560 mg, 5 mmol), 2-chloro-4-methoxypyrimidine (725 mg, 5 mmol) and potassium carbonate (2.07 mg, 15 mmol) in dioxane (20 mL) and water (10 mL) was added 1,1′-bis(diphenylphosphino)ferrocene-palladium(II)dichloride dichloromethane complex (409 mg, 0.5 mmol) and the resultant mixture was stirred at 100° C. for 4 h under argon. The mixture was then concentrated and purified by flash chromatography (Biotage, 40 g silica gel, dichloromethane=1) to give 2-(furan-3-yl)-4-methoxypyrimidine as white solid (700 mg, 66%); LCMS: [M+H]+=177.1.

### Step 2: Synthesis of 2-(furan-3-yl)pyrimidin-4-ol hydrochloride

A mixture of 2-(furan-3-yl)-4-methoxypyrimidine (524 mg, 3.0 mmol) and hydrochloric acid (6 N, 5 mL) was stirred at 100° C. for 2 h. The mixture was concentrated to afford 2-(furan-3-yl)pyrimidin-4-ol hydrochloride (790 mg, crude) as a yellow solid. LCMS: [M+H]+=163.1.

### Step 3: Synthesis of 4-chloro-2-(furan-3-yl)pyrimidine

A mixture of 2-(furan-3-yl)pyrimidin-4-ol hydrochloride (590 mg, 3.0 mmol) in phosphorus oxychloride (5 mL) was stirred at 120° C. for 2 h. The mixture was concentrated, the residue was diluted with water (50 mL) and neutralized with sodium bicarbonate to pH=8-9. The mixture was then extracted with ethyl acetate (100 mL*2), the organics layer was dried over anhydrous sodium sulfate, filtered and concentrated under reduced pressure to afford 4-chloro-2-(furan-3-yl)pyrimidine (600 mg, crude) as a yellow solid. LCMS: [M+H]+ 180.1.

### Step 4: Synthesis of 2-(furan-3-yl)-4-(trimethylstannyl)pyrimidine

A mixture of 4-chloro-2-(furan-2-yl)pyrimidine (180 mg, 1.0 mmol), hexamethyldistannane (490 mg, 1.5 mmol), bis(triphenylphosphine)palladium(II) chloride (71 mg, 0.1 mmol) and dioxane (10 mL) was stirred at 100° C. for 2 h under nitrogen atmosphere. The mixture was poured into dichloromethane (200 mL), the organic phase was washed successively with saturated potassium fluoride aqueous (100 mL), brine and concentrated to afford the crude 2-(furan-2-yl)-4-(trimethylstannyl)pyrimidine (250 mg, crude) as a brown oil. LCMS: [M+H]⁺ 310.8.

### Step 5: Synthesis of 4-(2-(2-(furan-3-yl)pyrimidin-4-yl)-9-methyl-8-(pyridin-4-yl)-9H-purin-6-yl)morpholine

To a solution of 2-(furan-3-yl)-4-(trimethylstannyl)pyrimidine (280 mg, 0.9 mmol) and 4-(2-chloro-9-methyl-8-(pyridin-4-yl)-9H-purin-6-yl)morpholine (298 mg, 0.9 mmol) in dioxane (10 mL) was added tetrakis(triphenylphosphin)palladium (104 mg, 0.09 mmol). The mixture was stirred at 100° C. for 2 h under argon and concentrated. The resultant crude product was purified by Prep-HPLC (BOSTON pHlex ODS 10 μm 21.2×250 mm120 A. The mobile phase was acetonitrile/0.1% Formic acid); then further purified by Prep-HPLC (BOSTON pHlex ODS 10 μm 21.2×250 mm120 A. The mobile phase was acetonitrile/0.1% Ammonium bicarbonate) to obtain 4-(2-(2-(furan-3-yl)pyrimidin-4-yl)-9-methyl-8-(pyridin-4-yl)-9H-purin-6-yl)morpholine as white solid (14.2 mg, 3.2%).

1H NMR (500 MHz, DMSO-d₆) δ 8.95 (d, J=5.1 Hz, 1H), 8.81 (d, J=6.0 Hz, 2H), 8.50 (s, 1H), 8.25 (d, J=5.1 Hz, 1H), 7.96 (d, J=6.0 Hz, 2H), 7.86 (s, 1H), 7.14 (s, 1H), 4.40 (s, 4H), 4.05 (s, 3H), 3.85-3.79 (m, 4H); LCMS: [M+H]+ 441.1.

### Synthesis of 4-(9-ethyl-2-(5-methoxy-2-phenylpyrimidin-4-yl)-8-(pyridin-4-yl)-9H-purin-6-yl)morpholine (Compound 4) and 4-(9-ethyl-6-morpholino-8-(pyridin-4-yl)-9H-purin-2-yl)-2-phenylpyrimidin-5-ol (Compound 5)

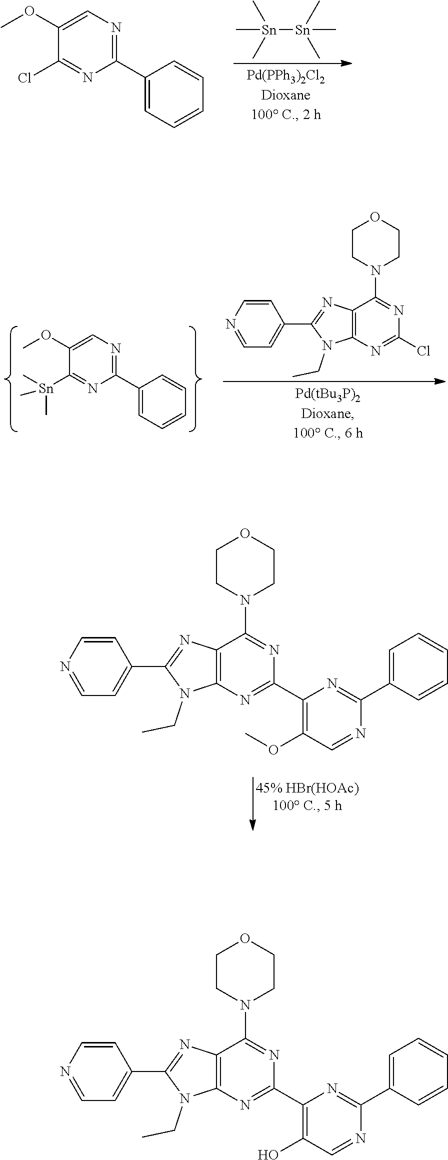

### Step 1: 4-(9-ethyl-2-(5-methoxy-2-phenylpyrimidin-4-yl)-8-(pyridin-4-yl)-9H-purin-6-yl)morpholine

A mixture of 4-chloro-5-methoxy-2-phenylpyrimidine (320 mg, 1.45 mmol), hexamethyldistannane (720 mg, 2.2 mmol), bis(triphenylphosphine)palladium(II) chloride (71 mg, 0.1 mmol) and dioxane (10 mL) was stirred at 100° C. for 2 h under nitrogen atmosphere. The mixture was poured into dichloromethane (200 mL), the organic phase was washed successively with saturated potassium fluoride aqueous (100 mL), brine and concentrated to afford the crude 5-methoxy-2-phenyl-4-(trimethylstannyl)pyrimidine (500 mg) as a brown oil. 100 mg of this product was mixed with 4-(2-chloro-9-ethyl-8-(pyridin-4-yl)-9H-purin-6-yl)morpholine (80 mg, 0.23 mmol), bis(tri-tert-butylphosphine)palladium (52 mg, 0.1 mmol) in dioxane (5 mL) and stirred at 100° C. for another 6 h and concentrated. The crude product thus obtained was purified by silica gel column chromatography to afford 4-(9-ethyl-2-(5-methoxy-2-phenylpyrimidin-4-yl)-8-(pyridin-4-yl)-9H-purin-6-yl)morpholine (180 mg, 82% purity) as a yellow solid. ¹H NMR (400 MHz, CDCl₃) δ 8.85-8.77 (m, 2H), 8.62 (s, 1H), 8.40 (dd, J=7.8, 1.5 Hz, 2H), 7.74-7.67 (m, 2H), 7.50-7.41 (m, 3H), 4.49 (q, J=7.2 Hz, 2H), 4.41 (bs, 4H), 3.98 (s, 3H), 3.90-3.83 (m, 4H), 1.48 (t, J=7.2 Hz, 3H); LCMS (ESI) m/z: 494.8 [M+H]⁺.

### Step 2: Synthesis of 4-(9-ethyl-6-morpholino-8-(pyridin-4-yl)-9H-purin-2-yl)-2-phenylpyrimidin-5-ol

A mixture of 4-(9-ethyl-2-(5-methoxy-2-phenylpyrimidin-4-yl)-8-(pyridin-4-yl)-9H-purin-6-yl)morpholine (100 mg, 0.2 mmol) in hydrobromic acid (45% in acetic acid, 6 mL) was stirred at 100° C. for 4 h. The formed precipitate was collected by filtration and purified by prep-HPLC (Column Xbridge 21.2*250 mm C18, 10 um, Mobile Phase A: water (10 mmol/L ammonium bicarbonate) B: acetonitrile) to afford 4-(9-ethyl-6-morpholino-8-(pyridin-4-yl)-9H-purin-2-yl)-2-phenylpyrimidin-5-ol (28.4 mg, 60%) as a light yellow solid.

¹H NMR (400 MHz, CDCl₃) δ 13.43 (s, 1H), 8.85 (dd, J=4.5, 1.6 Hz, 2H), 8.71 (s, 1H), 8.55-8.47 (m, 2H), 7.72 (dd, J=4.5, 1.6 Hz, 2H), 7.55-7.42 (m, 3H), 4.72-4.30 (m, 6H), 4.04-3.88 (m, 4H), 1.60 (t, J=7.2 Hz, 3H); LCMS (ESI) m/z: 480.8 [M+H]⁺.

### Synthesis of 4-(9-ethyl-8-(1-methyl-1H-pyrazol-5-yl)-2-(2-phenylpyrimidin-4-yl)-9H-purin-6-yl)-3-methylmorpholine (Compound 6)

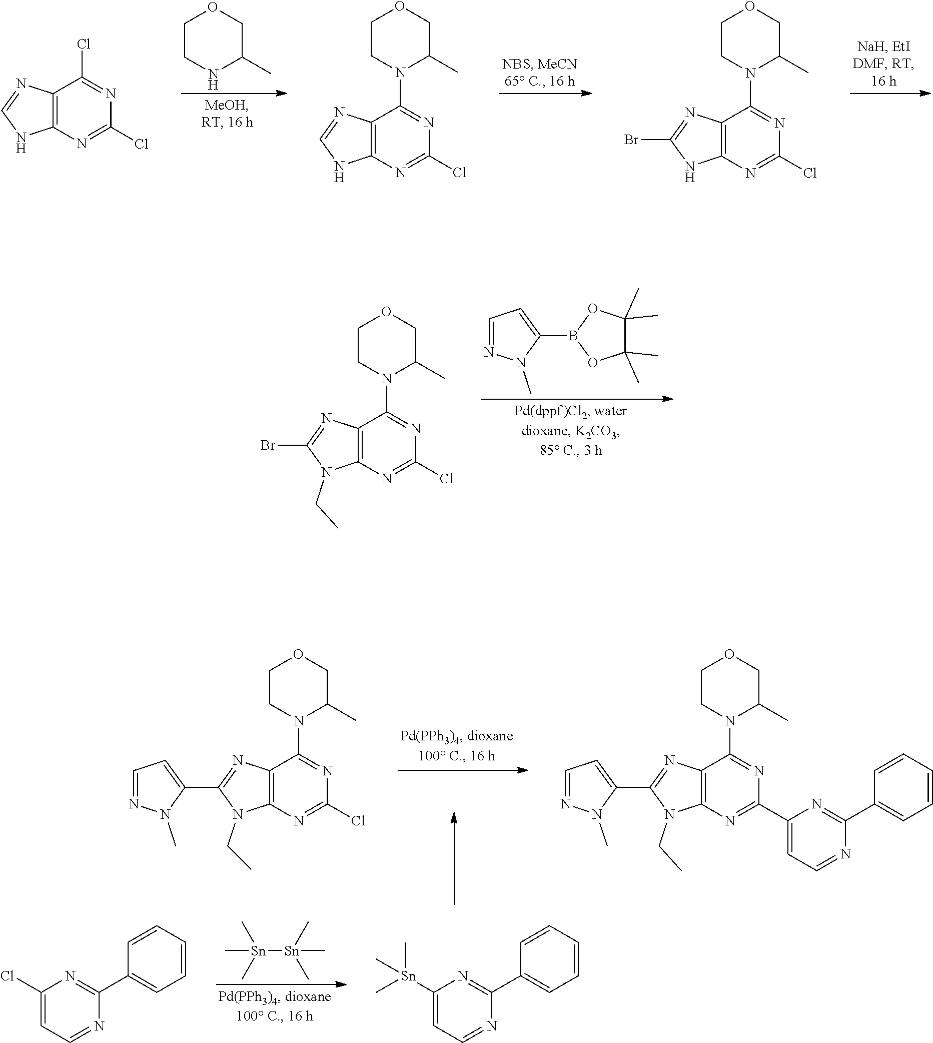

### Step 1: 4-(2-chloro-9H-purin-6-yl)-3-methylmorpholine

A mixture of 2,6-dichloro-9H-purine (5 g, 24.6 mmol) and 3-methylmorpholine (4 g, 39.7 mmol) in methanol (50 mL) was stirred at room temperature for 16 h. The mixture was evaporated and water (100 mL) was added. The aqueous layer was extracted with ethyl acetate (100 mL×4) which was dried and concentrated to afford the target compound (0.6 g, 9%) as white solid. LCMS (ESI) m/z: 254.1 [M+H]⁺.

### Step 2: 4-(8-bromo-2-chloro-9H-purin-6-yl)-3-methylmorpholine

A mixture of 4-(2-chloro-9H-purin-6-yl)-3-methylmorpholine (612 mg, 2.4 mmol) and N-bromosuccinimide (861 mg, 4.8 mmol) in acetonitrile (6 mL) was stirred at 65° C. for 16 h. The mixture was filtered, and the filtrate was triturated with acetonitrile to afford the target compound (0.5 g, 62%) as a white solid. LCMS (ESI) m/z: 334.0 [M+H]⁺.

### Step 3: 4-(8-bromo-2-chloro-9-ethyl-9H-purin-6-yl)-3-methylmorpholine

To a solution of 4-(8-bromo-2-chloro-9H-purin-6-yl)-3-methylmorpholine (440 mg, 1.32 mmol) and sodium hydride (58 mg, 1.45 mmol) in N,N-Dimethylformamide (5 mL) was added iodoethane (413 mg, 2.65 mmol) under ice-bath, and the mixture was stirred at 0˜25° C. for 2.0 h. The mixture was then extracted with ethyl acetate (20 mL*2) and washed with water (10 mL*2). The organic layer was dried over sodium sulfate, and concentrated. The crude product thus obtained was purified by silica gel column chromatography (10% methanol in dichlomethane) to give the title compound as white solid (450 mg, 94%). LCMS (ESI) m/z: 360.0 [M+H]⁺.

### Step 4: 4-(2-chloro-9-ethyl-8-(1-methyl-1H-pyrazol-5-yl)-9H-purin-6-yl)-3-methylmorpholine

A mixture of 4-(8-bromo-2-chloro-9-ethyl-9H-purin-6-yl)-3-methylmorpholine (150 mg, 0.42 mmol), potassium carbonate (86 mg, 0.625 mmol), [1,1′-bis(diphenylphosphino)ferrocene]dichloropalladium(II) (30 mg, 0.042 mmol) and 1-methyl-5-(4,4,5,5-tetramethyl-1,3,2-dioxaborolan-2-yl)-1H-pyrazole (173 mg, 0.83 mmol) in dioxane (5 mL) and water (0.5 mL) was stirred at 80° C. under nitrogen for 16 h. The mixture was extracted with ethyl acetate (20 mL*2) and washed with water (10 mL*2). The organic layer was dried over sodium sulfate, and concentrated. The residual was purified by silica gel column chromatography (10% methanol in dichlomethane) to give the title product as white solid (110 mg, 72%). LCMS (ESI) m/z: 361.8 [M+H]⁺.

### Step 4a: 2-phenyl-4-(trimethylstannyl)pyrimidine

To a solution of 4-chloro-2-phenylpyrimidine (1 g, 5.26 mmol) in dioxane (10 mL) was added 1,1,1,2,2,2-hexamethyldistannane (3.4 g, 10.5 mmol) and bis(triphenylphosphine)palladium(II) chloride (370 mg, 0.52 mmol) at 25° C. and the reaction was stirred at 100° C. for 5 h under strict argon atmosphere. An aqueous solution of potassium fluoride (500 mL) was added and stirred, the mixture was filtered, then the filtrate was extracted with dichloromethane (100 mL*3). The organic layer was dried and concentrated to get title product (1.6 g, 99%) as a brown oil. LCMS (ESI) m/z: 320.9 [M+H]⁺.

### Step 5: 4-(9-ethyl-8-(1-methyl-1H-pyrazol-5-yl)-2-(2-phenylpyrimidin-4-yl)-9H-purin-6-yl)-3-methylmorpholine

A mixture of 4-(2-chloro-9-ethyl-8-(1-methyl-1H-pyrazol-5-yl)-9H-purin-6-yl)-3-methylmorpholine (110 mg, 0.3 mmol), 2-phenyl-4-(trimethylstannyl)pyrimidine (145 mg, 3.3 mmol), tetratriphenylphosphonium palladium (34 mg, 0.03 mmol) in dioxane (2 mL) was stirred at 100° cunder nitrogen protection for 16 h. The crude product was purified by flash chromatography on silica gel (Petroleum ether/Ethyl acetate 20:1→10:1→5:1) to give the 4-(9-ethyl-8-(1-methyl-1H-pyrazol-5-yl)-2-(2-phenylpyrimidin-4-yl)-9H-purin-6-yl)-3-methylmorpholine (30.5 mg, 21%) as a white solid. 1H NMR (400 MHz, DMSO-d₆) δ 9.07 (d, J=5.1 Hz, 1H), 8.55 (dd, J=7.2, 2.3 Hz, 2H), 8.30 (d, J=5.1 Hz, 1H), 7.70 (d, J=2.0 Hz, 1H), 7.66-7.50 (m, 3H), 6.93 (d, J=2.0 Hz, 1H), 5.57 (bs, 1H), 5.17 (bs, 1H), 4.43 (q, J=7.3 Hz, 2H), 4.25-4.05 (m, 4H), 3.84 (d, J=7.2 Hz, 1H), 3.78 (d, J=7.2 Hz, 1H), 3.61 (d, J=11.4 Hz, 2H), 1.41 (dd, J=8.8, 7.1 Hz, 6H); LCMS (ESI) m/z: 482.0 [M+H]⁺.

The following compounds were synthesized according to the protocol described above.

| Name | Structure | NMR, MS | # |
|---|---|---|---|
| 4-(9-ethyl-2-(2- phenylpyrimidin-4- yl)-8-(pyridin-4-yl)- 9H-purin-6- yl)morpholine | 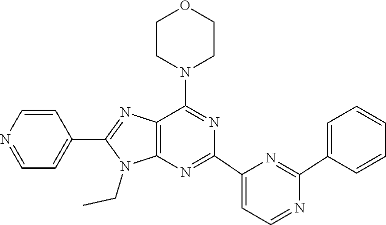 [SMILES: CCn1c(-c2ccncc2)nc2c(N3CCOCC3)nc(-c3ccnc(-c4ccccc4)n3)nc21] | 1H NMR (400 MHz, DMSO-d₆) δ 9.07 (d, J = 5.1 Hz, 1H), 8.83 (d, J = 5.7 Hz, 2H), 8.56 (dd, J = 7.3, 2.1 Hz, 2H), 8.33 (d, J = 5.1 Hz, 1H), 7.89 (d, J = 6.0 Hz, 2H), 7.71-7.49 (m, 3H), 4.54 (q, J = 7.2 Hz, 2H), 4.41 (bs, 4H), 3.87-3.75 (m, 4H), 1.41 (t, J = 7.2 Hz, 3H); LCMS (ESI) m/z: 465.0 [M + H]⁺ | 7 |
| 3-methyl-4-(9- methyl-2-(2- phenylpyrimidin-4- yl)-8-(pyridin-4-yl)- 9H-purin-6- yl)morpholine | 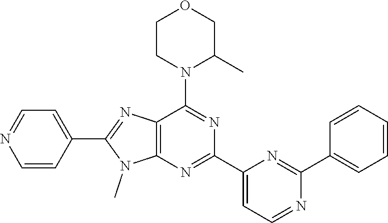 [SMILES: CC1COCCN1c1nc(-c2ccnc(-c3ccccc3)n2)nc2c1nc(-c1ccncc1)n2C] | 1H NMR (400 MHz, DMSO) δ 9.07 (d, J = 5.1 Hz, 1H), 8.82 (d, J = 5.9 Hz, 2H), 8.56 (dd, J = 7.4, 2.3 Hz, 2H), 8.33 (d, J = 5.1 Hz, 1H), 7.96 (dd, J = 4.6, 1.5 Hz, 2H), 7.67-7.51 (m, 3H), 5.62 (s, 2H), 4.06 (s, 4H), 3.92- 3.72 (m, 2H), 3.61 (d, J = 11.3 Hz, 2H), 1.42 (d, J = 6.7 Hz, 3H); LCMS (ESI) m/z: 465.7 [M + H]+. | 8 |

### Synthesis of 4-(9-methyl-2-(5-methyl-2-phenylpyrimidin-4-yl)-8-(pyridin-4-yl)-9H-purin-6-yl)morpholine (Compound 9)

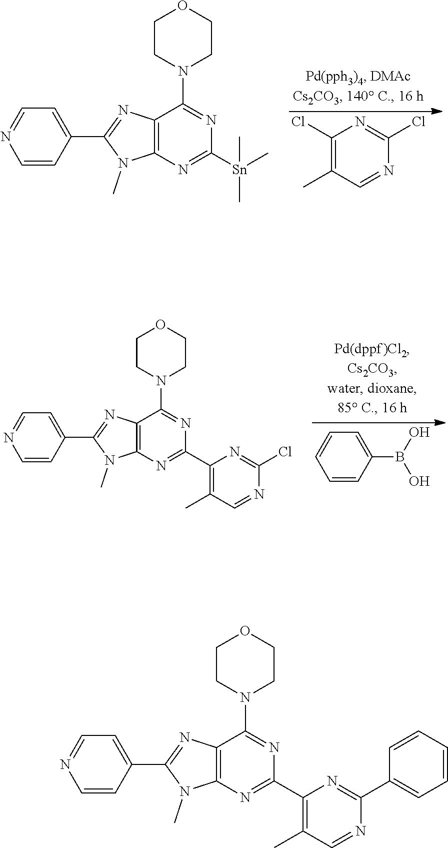

### Step 1: 4-(2-(2-chloro-5-methylpyrimidin-4-yl)-9-methyl-8-(pyridin-4-yl)-9H-purin-6-yl)morpholine

To a solution of 4-(9-methyl-8-(pyridin-4-yl)-2-(trimethylstannyl)-9H-purin-6-yl)morpholine (30 mg, 0.06 mmol) in dioxane (5 mL) was added 2,4-dichloro-5-methylpyrimidine (100 mg, 0.06 mmol) and tetrakis(triphenylphosphine)palladium (1 mg, 0.006 mmol) at 25° C. and the reaction mixture was stirred at 100° C. for 17 h under nitrogen atmosphere. The reaction mixture was then diluted with water (30 mL) and the resulting mixture was extracted with dichloromethane (30 mL×3). The combined organic layers were washed with saturated aqueous brine (20 mL), dried over anhydrous sodium sulfate and concentrated in vacuo to give the desired product (50 mg, 99%). LCMS (ESI) m/z: 423.7 [M+H]⁺.

### Step 2: 4-(9-methyl-2-(5-methyl-2-phenylpyrimidin-4-yl)-8-(pyridin-4-yl)-9H-purin-6-yl)morpholine

A mixture of 4-(2-(2-chloro-5-methylpyrimidin-4-yl)-9-methyl-8-(pyridin-4-yl)-9H-purin-6-yl)morpholine (50 mg, 0.12 mmol), phenylboronic acid (21 mg, 0.17 mmol), [1,1′-Bis(diphenylphosphino)ferrocene]dichloropalladium(II) (96 mg, 0.3 mmol) and cesium carbonate (96 mg, 2.5 mmol) in dioxane (5 mL) and water (0.5 mL) was stirred at 85° C. for 16 h under argon atmosphere. The mixture was concentrated and crude product was chromatographed on silica gel (Petroleum ether/Ethyl acetate 20:1→10:1→5:1) to obtain 4-(9-methyl-2-(5-methyl-2-phenylpyrimidin-4-yl)-8-(pyridin-4-yl)-9H-purin-6-yl)morpholine (40 mg, 72%) as white solid.

1H NMR (400 MHz, DMSO-d₆) δ 8.90 (s, 1H), 8.81 (d, J=6.1 Hz, 2H), 8.41 (dd, J=6.6, 3.2 Hz, 2H), 7.95 (d, J=6.1 Hz, 2H), 7.53 (d, J=2.2 Hz, 3H), 4.34 (s, 4H), 3.97 (s, 3H), 3.78 (s, 4H), 2.40 (s, 3H); LCMS (ESI) m/z: 465.7 [M+H]+

The following compounds were prepared according to the protocol described above:

| Name | Structure | NMR, MS | # |
|---|---|---|---|
| 4-(2-(2-(3- methoxyphenyl) pyrimidin-4-yl)-9- methyl-8-(pyridin- 4-yl)-9H-purin-6- yl)morpholine | 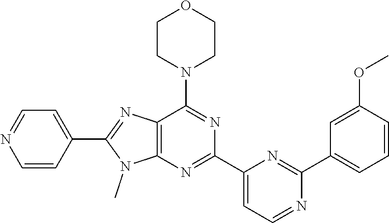 [SMILES: COc1cccc(-c2nccc(-c3nc(N4CCOCC4)c4nc(-c5ccncc5)n(C)c4n3)n2)c1] | ¹H NMR (400 MHz, DMSO-d₆) δ 9.06 (t, J = 4Hz, 1H), 8.81 (d, J = 4 Hz, 2H), 8.14 (dd, J = 8, 4 Hz, 2H), 7.95 (d, J = 4.6 Hz, 2H), 7.50 (t, J = 7.9 Hz, 1H), 7.14 (dd, J = 8.1, 2.6 Hz, 1H), 4.42 (s, 4H), 4.05 (s, 3H), 3.88 (s, 3H), 3.86-3.78 (m, 4H); LCMS (ESI) m/z: 481.2 [M + H]⁺. | 10 |
| 4-(2-(2-(furan-2- yl)pyrimidin-4-yl)- 9-methyl-8- (pyridin-4-yl)-9H- purin-6- yl)morpholine | 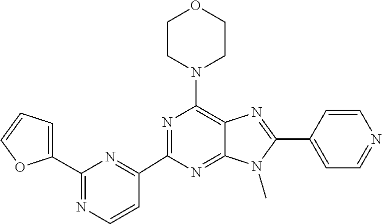 [SMILES: Cn1c(-c2ccncc2)nc2c(N3CCOCC3)nc(-c3ccnc(-c4ccco4)n3)nc21] | ¹H NMR (400 MHz, CDCl₃) δ 8.91 (d, J = 5.1 Hz, 1H), 8.83 (d, J = 5.8 Hz, 2H), 8.24 (d, J = 5.1 Hz, 1H), 7.79 (d, J = 6.0 Hz, 2H), 7.68 (d, J = 0.8 Hz, 1H), 7.48 (d, J = 3.0 Hz, 1H), 6.61 (dd, J = 3.4, 1.7 Hz, 1H), 4.55 (bs, 4H), 4.13 (s, 3H), 3.97-3.87 (m, 4H). LCMS (ESI) m/z: 440.7 [M + H]⁺. | 11 |
| 4-(2-(4- cyclopropylpyrimidin- 2-yl)-9- methyl-8-(pyridin- 4-yl)-9H-purin-6- yl)morpholine | 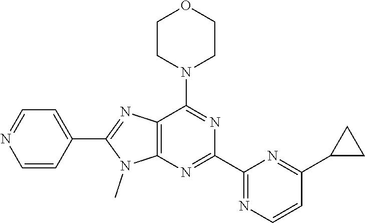 [SMILES: Cn1c(-c2ccncc2)nc2c(N3CCOCC3)nc(-c3nccc(C4CC4)n3)nc21] | 1H NMR (400 MHz, CD₃OD) δ 8.75 (d, J = 1.6 Hz, 2H), 8.70 (d, J = 5.3 Hz, 1H), 7.99 (dd, J = 4.6, 1.6 Hz, 2H), 7.36 (d, J = 5.3 Hz, 1H), 4.46 (s, 4H), 4.08 (s, 3H), 3.96-3.76 (m, 4H), 2.34-2.14 (m, 1H), 1.6- 1.25 (m, 5H); LCMS (ESI) m/z: 415.7 [M + H]⁺ | 12 |
| 4-(9-ethyl-2-(2- phenylpyrimidin- 4-yl)-8-(pyridin-4- yl)-9H-purin-6-yl)- 3- methylmorpholine | 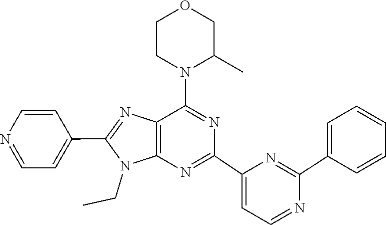 [SMILES: CCn1c(-c2ccncc2)nc2c(N3CCOCC3C)nc(-c3ccnc(-c4ccccc4)n3)nc21] | 1H NMR (400 MHz, DMSO-d₆) δ 9.07 (d, J = 5.1 Hz, 1H), 8.84 (s, 2H), 8.58-8.54 (m, 2H), 8.31 (d, J = 5.1 Hz, 1H), 7.89 (d, J = 5.7 Hz, 2H), 7.61-7.56 (m, 3H), 5.55 (s, 2H), 4.53 (q, J = 7.3 Hz, 2H), 4.06 (d, J = 8.1 Hz, 1H), 3.85 (d, J = 11.4 Hz, 1H), 3.77 (dd, J = 11.5, 2.9 Hz, 1H), 3.66-3.47 (m, 2H), 1.44-1.39 (m, 6H); LCMS (ESI) m/z: 479.1 [M + H]⁺ | 13 |

The following compounds were synthesized according the protocol described for the Compound 2.

| Name | Structure | 1H NMR Data | # |
|---|---|---|---|
| 4-(9-methyl-2-(2- phenylpyrimidin- 4-yl)-8-(pyridin-4- yl)-9H-purin-6- yl)morpholine | 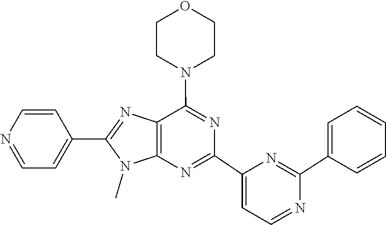 [SMILES: Cn1c(-c2ccncc2)nc2c(N3CCOCC3)nc(-c3ccnc(-c4ccccc4)n3)nc21] | ¹H NMR (500 MHz, Chloroform-d) δ 8.97 (d, J = 5.1 Hz, 1H), 8.87-8.79 (m, 2H), 8.66-8.58 (m, 2H), 8.31 (d, J = 5.1 Hz, 1H), 7.82-7.74 (m, 2H), 7.57-7.48 (m, 3H), 4.51 (s, 4H), 4.13 (s, 3H), 3.97-3.89 (m, 4H); LCMS (ESI) m/z: 451.2 [M + H]⁺. | 14 |
| 4-(9-methyl-2-(6- (piperidin-4- yl)pyridin-2-yl)-8- (pyridin-4-yl)-9H- purin-6- yl)morpholine | 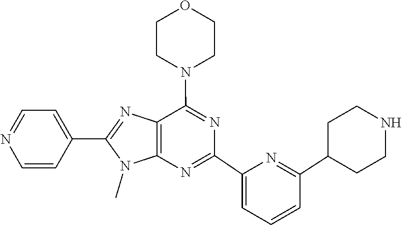 [SMILES: Cn1c(-c2ccncc2)nc2c(N3CCOCC3)nc(-c3cccc(C4CCNCC4)n3)nc21] | ¹H NMR (400 MHz, Dimethylsulfoxide-d₆) δ 8.80 (dd, J = 4.5, 1.6 Hz, 2H), 8.20 (d, J = 7.4 Hz, 1H), 7.94 (dd, J = 4.5, 1.6 Hz, 2H), 7.85 (t, J = 7.8 Hz, 1H), 7.34 (d, J = 7.6 Hz, 1H), 4.36 (s, 4H), 4.00 (s, 3H), 3.87-3.71 (m, 4H), 3.07 (d, J = 11.6 Hz, 2H), 2.86 (t, J = 12.0 Hz, 1H), 2.64 (t, J = 11.5 Hz, 2H), 1.84 (d, J = 11.7 Hz, 2H), 1.69 (qd, J = 12.3, 3.8 Hz, 2H); LCMS (ESI) m/z: 457.2 [M + H]⁺. | 15 |
| 4-(9-methyl-2-(2- (piperidin-4- yl)pyrimidin-4-yl)- 8-(pyridin-4-yl)- 9H-purin-6- yl)morpholine | 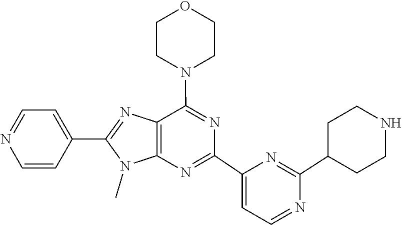 [SMILES: Cn1c(-c2ccncc2)nc2c(N3CCOCC3)nc(-c3ccnc(C4CCNCC4)n3)nc21] | ¹H NMR (400 MHz, DMSO- d₆) δ 8.90 (d, J = 4.8 Hz, 1H), 8.82-8.80 (m, 2H), 8.20 (d, J = 5.2 Hz, 1H), 7.95-7.94 (m, 2H), 4.40-4.33 (m, 4H), 4.01 (s, 3H), 3.81-3.79 (m, 4H), 3.10-3.02 (m, 3H), 2.71-2.65 (m, 2H), 1.95- 1.92 (m, 2H), 1.83-1.77 (m, 2H), LCMS (ESI) m/z: 458 [M + H]⁺. | 16 |
| tert-butyl 4-(6-(9- methyl-6- morpholino-8- (pyridin-4-yl)-9H- purin-2-yl)pyridin- 2-yl)piperidine-1- carboxylate | 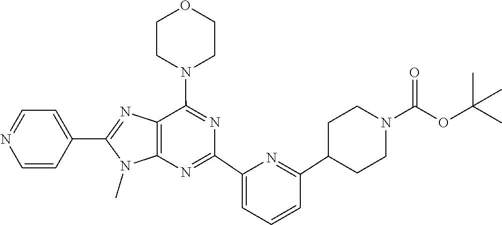 [SMILES: Cn1c(-c2ccncc2)nc2c(N3CCOCC3)nc(-c3cccc(C4CCN(C(=O)OC(C)(C)C)CC4)n3)nc21] | ¹H NMR (400 MHz, Dimethylsulfoxide-d₆) δ 8.80 (dd, J = 4.5, 1.6 Hz, 2H), 8.22 (d, J = 7.2 Hz, 1H), 7.94 (dd, J = 4.5, 1.6 Hz, 2H), 7.86 (t, J = 7.8 Hz, 1H), 7.38 (d, J = 7.3 Hz, 1H), 4.36 (s, 4H), 4.09 (d, J = 12.5 Hz, 2H), 4.00 (s, 3H), 3.86-3.70 (m, 4H), 2.99 (t, J = 11.7 Hz, 3H), 1.91 (d, J = 11.3 Hz, 2H), 1.68 (dt, J = 12.2, 8.4 Hz, 2H), 1.55- 1.21 (m, 9H); LCMS (ESI) m/z: 557.3 [M + H]⁺. | 17 |
| 4-(9-methyl-8- (pyridin-4-yl)-2- (2- (trifluoromethyl) pyrimidin-4-yl)-9H- purin-6- yl)morpholine | 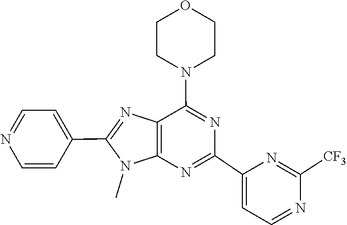 [SMILES: Cn1c(-c2ccncc2)nc2c(N3CCOCC3)nc(-c3ccnc(C(F)(F)F)n3)nc21] | ¹H NMR (400 MHz, DMSO) δ 9.21 (d, J = 5.2 Hz, 1H), 8.81 (d, J = 5.6 Hz, 2H), 8.73 (d, J = 5.2 Hz, 1H), 7.95 (d, J = 5.7 Hz, 2H), 4.38 (bs, 4H), 4.03 (s, 3H), 3.87-3.73 (m, 4H). LCMS (ESI) m/z: 443.1[M + H]+. | 18 |
| 4-(2-(4- cyclopropylpyrimidin- 2-yl)-9- methyl-8-(pyridin- 4-yl)-9H-purin-6- yl)morpholine | 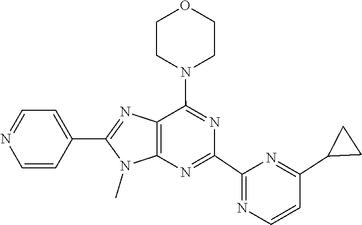 [SMILES: Cn1c(-c2ccncc2)nc2c(N3CCOCC3)nc(-c3nccc(C4CC4)n3)nc21] | 1H NMR (400 MHz, CD₃OD) δ 8.75 (d, J = 4 Hz, 2H), 8.70 (d, J = 5.3 Hz, 1H), 7.99 (dd, J = 4.6, 1.6 Hz, 2H), 7.36 (d, J = 5.3 Hz, 1H), 4.46 (s, 4H), 4.08 (s, 3H), 3.96- 3.76 (m, 4H), 2.26 (pent, J = 4 Hz, 1H), 1.30- 1.20 (m, 4H); LCMS (ESI) m/z: 415.1 [M + H]⁺ | 19 |

### Synthesis of 4-(2-(2-cyclopropylpyrimidin-4-yl)-9-methyl-8-(pyridin-4-yl)-9H-purin-6-yl)morpholine (Compound 20)

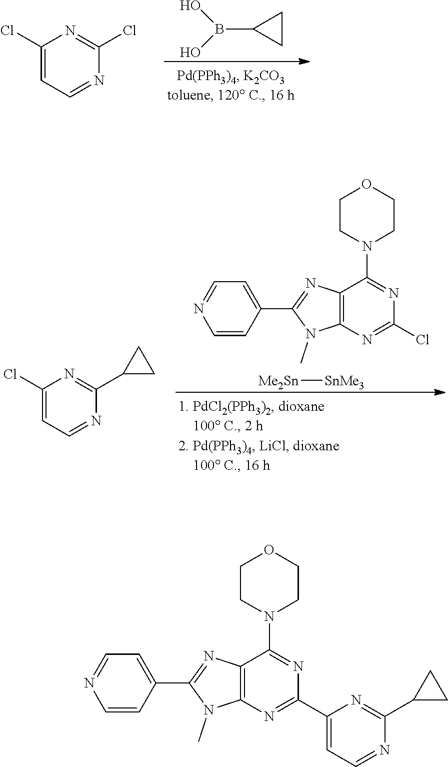

### Step 1: Synthesis of 4-Chloro-2-cyclopropylpyrimidine

A solution of 2,4-dichloropyrimidine (500 mg, 3.355 mmol), cyclopropylboronic acid (288 mg, 3.355 mmol), tetrakis(triphenyl phosphine)palladium (352 mg, 0.3355 mmol) and potassium carbonate (1389 mg, 10.065 mmol) in dioxane (30 mL) was stirred at 100° C. for 16 h. Then water was added and the mixture was extracted with ethyl acetate (50 mL×3). The organic layer was dried and concentrated and the crude product was purified by Pre-TLC (petroleum ether:ethyl acetate from 50:1 to 10:1) to give 4-chloro-2-cyclopropylpyrimidine (310 mg, 60%) as a yellow solid. LC-MS: m/z=155 (M+H)⁺.

### Step 2: Synthesis of 4-(9-Methyl-8-(pyridin-4-yl)-2-(trimethylstannyl)-9H-purin-6-yl)morpholine

A solution of 4-(2-chloro-9-methyl-8-(pyridin-4-yl)-9H-purin-6-yl)morpholine (600 mg, 1.812 mmol), 1,1,1,2,2,2-hexamethyl distannane (1185 mg, 3.625 mmol), bis(triphenylphosphine)palladium(II) dichloride (127 mg, 0.181 mmol) in dioxane (25 mL) was stirred at 100° C. for 1 h. To the resultant mixture were added, 4-chloro-2-cyclopropylpyrimidine (250 mg, 1.623 mmol), tetrakis(triphenylphosphine)palladium (170 mg, 0.162 mmol) and lithium chloride (136 mg, 3.246 mmol) in dioxane (30 mL) and the resultant mixture was stirred at 100° C. for 16 h. It was concentrated and the crude product was purified by silica gel column (dichloromethane:methanol from 100:1 to 10:1) to afford 4-(2-(2-cyclopropylpyrimidin-4-yl)-9-methyl-8-(pyridin-4-yl)-9H-purin-6-yl)morpholine (6.3 mg, 1%) as white solid.

¹H NMR (400 MHz, CD₃OD) δ 8.79-8.76 (m, 3H), 8.24 (d, J=5.2 Hz, 1H), 8.02 (d, J=4.8, 1.4 Hz, 2H), 4.48 (bs, 4H), 4.14 (s, 3H), 3.91-3.88 (m, 4H), 2.50-2.42 (m, 1H), 1.26-1.24 (m, 2H), 1.17-1.15 (m, 2H); LC-MS: m/z=415.2 (M+H)⁺.

### Synthesis of 4-(9-methyl-2-(2-(piperidin-3-yl)pyrimidin-4-yl)-8-(pyridin-4-yl)-9H-purin-6-yl)morpholine (Compound 21)

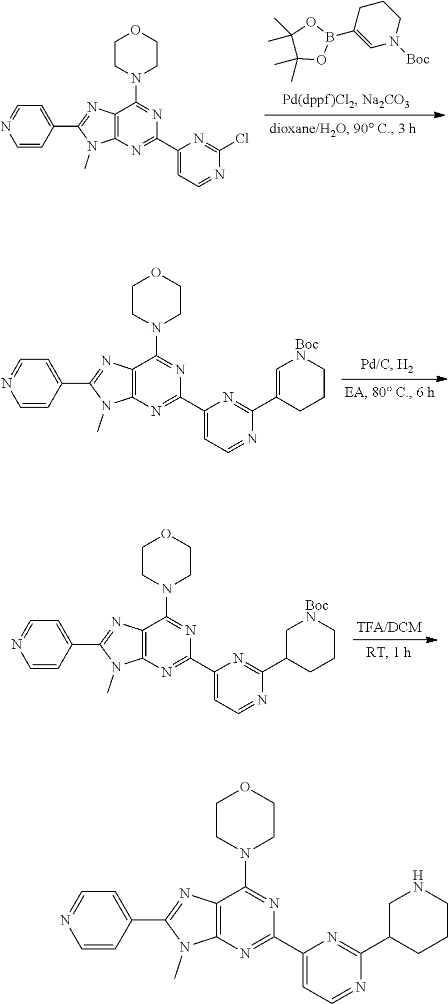 [SMILES: Cn1c(-c2ccncc2)nc2c(N3CCOCC3)nc(-c3ccnc(Cl)n3)nc21 ; CC(C)(C)OC(=O)N1C=C(B2OC(C)(C)C(C)(C)O2)CCC1 ; Cn1c(-c2ccncc2)nc2c(N3CCOCC3)nc(-c3ccnc(C4=CN(C(=O)OC(C)(C)C)CCC4)n3)nc21 ; Cn1c(-c2ccncc2)nc2c(N3CCOCC3)nc(-c3ccnc(C4CCCN(C(=O)OC(C)(C)C)C4)n3)nc21 ; Cn1c(-c2ccncc2)nc2c(N3CCOCC3)nc(-c3ccnc(C4CCCNC4)n3)nc21]

### Step 1: Preparation of tert-butyl 5-(4-(9-methyl-6-morpholino-8-(pyridin-4-yl)-9H-purin-2-yl)pyrimidin-2-yl)-3,4-dihydropyridine-1(2H)-carboxylate

A mixture of 4-(2-(2-chloropyrimidin-4-yl)-9-methyl-8-(pyridin-4-yl)-9H-purin-6-yl)morpholine (75 mg, 0.18 mmol), tert-butyl 5-(4,4,5,5-tetramethyl-1,3,2-dioxaborolan-2-yl)-3,4-dihydropyridine-1(2H)-carboxylate (62 mg, 0.20 mmol), Na₂CO₃ (58 mg, 0.55 mmol) and Pd(dppf)Cl₂ (15 mg, 0.2 mmol) in DMF (8 mL) and H₂O (1 mL) was stirred at 80° C. for 2 h under nitrogen protection. The mixture was concentrated and purified by column chromatography (20% EA in PE) to obtain tert-butyl 5-(4-(9-methyl-6-morpholino-8-(pyridin-4-yl)-9H-purin-2-yl)pyrimidin-2-yl)-3,4-dihydropyridine-1(2H)-carboxylate as white solid (60 mg, 59%). LCMS (ESI) m/z: 556 [M+H]⁺.

### Step 2: Preparation of tert-butyl 3-(4-(9-methyl-6-morpholino-8-(pyridin-4-yl)-9H-purin-2-yl)pyrimidin-2-yl)piperidine-1-carboxylate

A mixture of tert-butyl 5-(4-(9-methyl-6-morpholino-8-(pyridin-4-yl)-9H-purin-2-yl)pyrimidin-2-yl)-3,4-dihydropyridine-1(2H)-carboxylate (70 mg, 0.14 mmol) and 10% Pd/C (70 mg) in MeOH (5 mL) and ethyl acetate (5 mL) was stirred at 80° C. for 16 h under H₂ atmosphere. The mixture was filtered and concentrated to obtain the desired product as white solid (60 mg, 85%). LCMS (ESI) m/z: 558 [M+H]⁺.

### Step 3: Preparation of 4-(9-methyl-2-(2-(piperidin-3-yl)pyrimidin-4-yl)-8-(pyridin-4-yl)-9H-purin-6-yl)morpholine

To a solution of tert-butyl 3-(4-(9-methyl-6-morpholino-8-(pyridin-4-yl)-9H-purin-2-yl)pyrimidin-2-yl)piperidine-1-carboxylate (50 mg, 0.11 mmol) in DCM (5 mL) was added TFA (2 mL) and the mixture was stirred at room temperature for 1 h. The resultant mixture was concentrated and purified by Prep-HPLC to obtain 4-(9-methyl-2-(2-(piperidin-3-yl)pyrimidin-4-yl)-8-(pyridin-4-yl)-9H-purin-6-yl)morpholine. (2.3 mg, 4%) as white solid.

¹H NMR (400 MHz, DMSO-d₆) δ 8.91 (d, J=5.2 Hz, 1H), 8.81 (d, J=5.6 Hz, 2H), 8.22 (d, J=5.6 Hz, 1H), 7.95 (d, J=6.0 Hz, 2H), 4.43-4.31 (m, 4H), 4.02 (s, 3H), 3.81-3.77 (m, 4H), 3.40-3.35 (m, 1H), 3.11-2.92 (m, 3H), 2.89-2.60 (m, 1H), 2.44-2.15 (m, 1H), 2.11-1.54 (m, 3H); LCMS (ESI) m/z: 458.2 [M+H]⁺.

### Synthesis of 4-(9-(difluoromethyl)-2-(2-phenylpyrimidin-4-yl)-8-(pyridin-4-yl)-9H-purin-6-yl)morpholine (Compound 22)

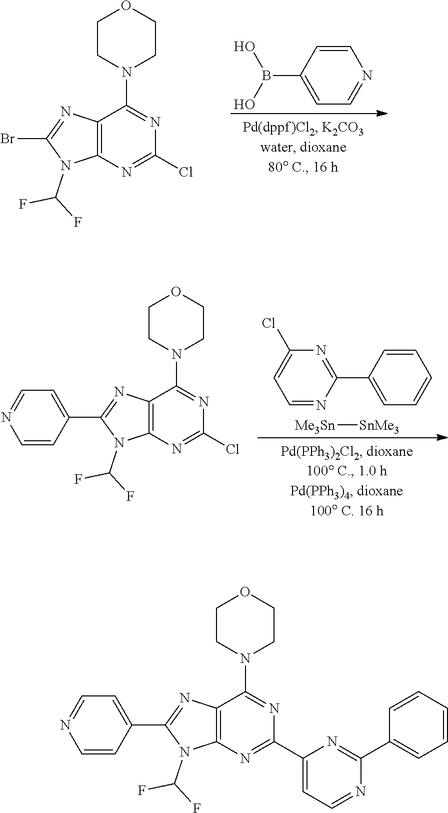

### Step 1: Synthesis of 4-(2-chloro-9-(difluoromethyl)-8-(pyridin-4-yl)-9H-purin-6-yl)morpholine

A solution of 4-(8-bromo-2-chloro-9-(difluoromethyl)-9H-purin-6-yl)morpholine (300 mg, 0.8 mmol), pyridin-4-ylboronic acid (108 mg, 0.88 mmol), 1,1′-bis(diphenylphosphino) ferrocene-palladium(II) dichloride dichloromethane complex (65 mg, 0.08 mmol) and potassium carbonate (330 mg, 2.4 mmol) in water (1.5 mL) and dioxane (15 mL) was stirred at 90° C. for 16 h under argon. The reaction mixture was cooled and concentrated. The crude product was purified by flash chromatography (Biotage, 80 g silica gel, methanol/dichloromethane=3%-4%) to give the desired product 4-(2-chloro-9-(difluoromethyl)-8-(pyridin-4-yl)-9H-purin-6-yl)morpholine (240 mg, 73%) as yellow solid. LCMS: (ESI) m/z 366.8 [M+H]+.

### Step 2: Synthesis of 4-(9-(difluoromethyl)-2-(2-phenylpyrimidin-4-yl)-8-(pyridin-4-yl)-9H-purin-6-yl)morpholine

To a solution of 4-chloro-2-phenylpyrimidine (92 mg, 0.5 mmol) in dioxane (10 mL) were added hexamethyldistannane (196 mg, 0.6 mmol) and bis(triphenylphosphine)palladium(II) chloride (35 mg, 0.05 mmol). The mixture was stirred at 100° C. for 1 h. The reaction mixture was cooled and 4-(2-chloro-9-(difluoromethyl)-8-(pyridin-4-yl)-9H-purin-6-yl)morpholine (92 mg, 0.25 mmol) and tetrakis(triphenylphosphine)palladium (58 mg, 0.05 mmol) were added to the reaction mixture and stirring was continued at 100° C. for 16 h. The reaction mixture was concentrated, the crude product was purified by Prep-HPLC (BOSTON pHlex ODS 10 μm 21.2×250 mm 120 A. The mobile phase was acetonitrile/0.1% Ammonium bicarbonate) to afford 4-(9-(difluoromethyl)-2-(2-phenylpyrimidin-4-yl)-8-(pyridin-4-yl)-9H-purin-6-yl)morpholine (23.3 mg, 13%) as white solid. 1H NMR (400 MHz, DMSO-d₆) δ 9.10 (d, J=5.1 Hz, 1H), 8.85 (d, J=6.0 Hz, 2H), 8.57 (dd, J=6.7, 3.0 Hz, 2H), 8.33 (d, J=5.2 Hz, 1H), 8.26 (t, J=58 Hz, 1H), 7.87 (d, J=6.0 Hz, 2H), 7.66-7.52 (m, 3H), 4.41 (s, 4H), 3.89-3.74 (m, 4H); LCMS: (ESI) m/z 486.8 [M+H]+.

### Synthesis of 4-(9-ethyl-2-(6-methoxy-5-phenylpyridazin-3-yl)-8-(pyridin-4-yl)-9H-purin-6-yl)morpholine (Compound 24)

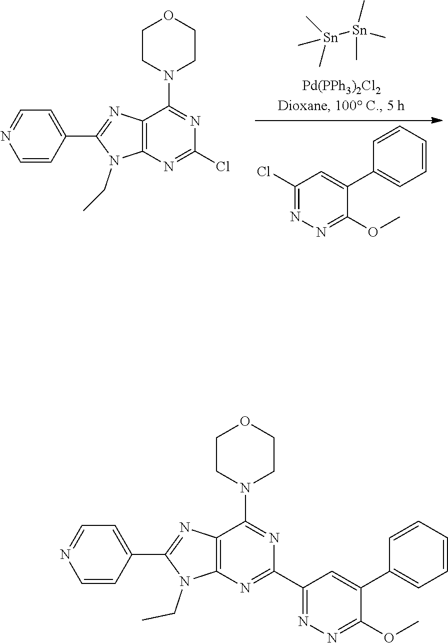

A solution of 4-(2-chloro-9-ethyl-8-(pyridin-4-yl)-9H-purin-6-yl)morpholine (93 mg, 0.27 mmol), 6-chloro-3-methoxy-4-phenylpyridazine (50 mg, 0.22 mmol), bis(triphenylphosphine)palladium(II) chloride (15 mg, 0.02 mmol) and hexamethyldistannane (143 mg, 0.44 mmol) in dioxane (5 mL) was stirred at 100° C. for 16 h under nitrogen. The reaction mixture was cooled to room temperature and treated with aq. Potassium fluoride (500 mL), stirred for 10 min and filtered. The filtrate was extracted with dichloromethane (100 mL*3) and the combined organic layer was concentrated. The residue was purified by flash chromatography (Dichloromethane/Methanol 20:1→10:1→5:1) to give 4-(9-ethyl-2-(6-methoxy-5-phenylpyridazin-3-yl)-8-(pyridin-4-yl)-9H-purin-6-yl)morpholine (5.7 mg, 5%) as a white solid.

¹H NMR (400 MHz, DMSO-d₆) δ 8.81 (d, J=6.0 Hz, 2H), 8.40 (s, 1H), 7.88 (d, J=6.1 Hz, 2H), 7.75 (d, J=6.4 Hz, 2H), 7.58-7.50 (m, 3H), 4.45 (q, J=7.2 Hz, 6H), 4.35 (bs, 4H), 4.17 (s, 3H), 3.79 (s, 4H), 1.37 (t, J=7.2 Hz, 3H); LCMS (ESI) m/z: 495.1 [M+H]⁺.

### Synthesis of 4-(9-ethyl-2-(4-phenylpyridazin-3-yl)-8-(pyridin-4-yl)-9H-purin-6-yl)morpholine (Compound 25)

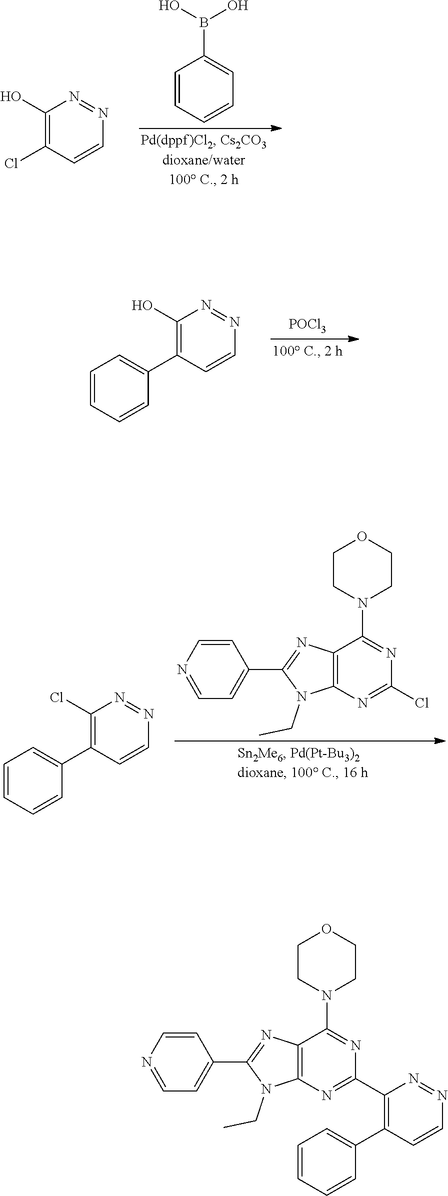

### Step 1: Synthesis of 4-phenylpyridazin-3-ol

To a solution of 4-chloropyridazin-3-ol (0.6 g, 4.6 mmol) and phenylboronic acid (0.56 g, 4.6 mmol) in dioxane/water (10 mL/3 mL) were added cesium carbonate (3 g, 9.2 mmol) and [1,1′-bis(diphenylphosphino)ferrocene]dichloropalladium(II) (0.34 g, 0.46 mmol) and the resultant mixture was stirred at 100° C. for 2 h. The mixture was poured into ice-water and extracted with ethyl acetate (15 mL*3), the organic layer was washed with brine, dried and evaporated to dryness. The crude product was chromatographed on silica gel (dichloromethane/Methanol 10:1) to give the desired product (400 mg, 51%) as a brown solid. LCMS (ESI) m/z: 173.1 [M+H]⁺.

### Step 2: Synthesis of 3-chloro-4-phenylpyridazine

A solution of 4-phenylpyridazin-3-ol (0.4 g, 2.0 mmol) in phosphorus oxychloride (10 mL) was stirred at 100° C. for 2 h under argon protection. The reaction was cooled, quenched with water (60 mL), adjusted pH to 7 with potassium carbonate and extracted with ethyl acetate (100 mL*5). The organics were combined and concentrated to give product as a brown solid (0.2 g, 45%). LCMS (ESI) m/z: 191.1 [M+H]⁺.

### Step 3: Synthesis of 4-(9-ethyl-2-(4-phenylpyridazin-3-yl)-8-(pyridin-4-yl)-9H-purin-6-yl)morpholine

To a solution of 4-(2-chloro-9-ethyl-8-(pyridin-4-yl)-9H-purin-6-yl)morpholine (0.18 g, 0.52 mmol) in dioxane (7 mL) was added hexamethyldistannane (0.24 g, 0.73 mmol) and tetrakis(triphenylphosphine)palladium (0.06 g, 0.052 mmol) at 25° C. and the reaction was stirred at 100° C. for 3 h under argon protection. The reaction was cooled to 25° C., followed by the addition of bis(tri-tert-butylphosphine)palladium(0) (0.027 g, 0.052 mmol), cesium fluoride (0.16 g, 1.4 mmol), cuprous iodide (0.01 g, 0.052 mmol) and 3-chloro-4-phenylpyridazine (0.12 g, 0.63 mmol) and the resultant mixture was stirred at 100° C. for another 16 h under argon protection. The entire mixture was concentrated and the crude product thus obtained was purified by Prep-HPLC (SunFire C18, 4.6*50 mm, 3.5 um column Xbridge C18 3.5 μm 4.6×50 mm column. The mobile phase was acetonitrile/0.1% Ammonium bicarbonate aqueous solution.) to give the desired product as off-white solid (28.6 mg, 11.8%). ¹H NMR (400 MHz, DMSO-d₆) δ 9.39 (d, J=5.3 Hz, 1H), 8.79 (dd, J=4.5, 1.5 Hz, 2H), 7.88-7.77 (m, 3H), 7.38-7.31 (m, 3H), 7.29-7.21 (m, 2H), 4.31 (q, J=7.1 Hz, 2H), 3.98 (s, 4H), 3.55 (s, 4H), 1.18 (t, J=7.2 Hz, 3H); LCMS (ESI) m/z: 465.1 [M+H]⁺.

### Synthesis of 4-(9-ethyl-2-(6-methyl-5-phenylpyridazin-3-yl)-8-(pyridin-4-yl)-9H-purin-6-yl)morpholine (Compound 26)

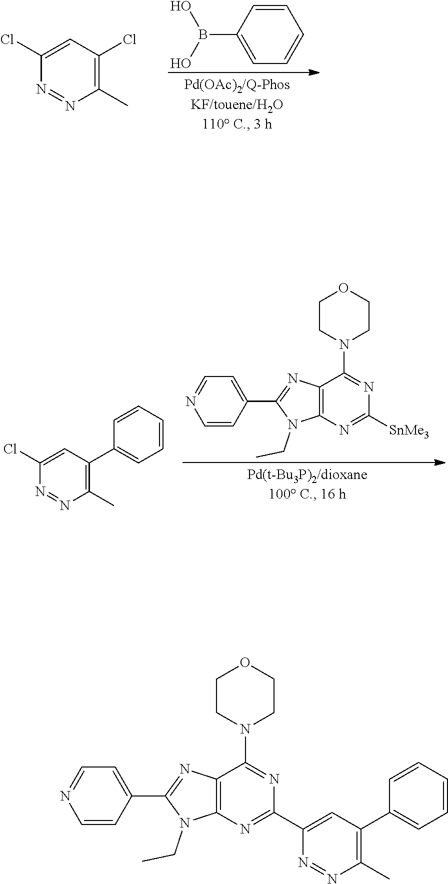

### Step 1: Synthesis of 6-chloro-3-methyl-4-phenylpyridazine

A mixture of 4,6-dichloro-3-methylpyridazine (486 mg, 3.0 mmol), phenylboronic acid (440 mg, 3.6 mmol), palladium (II) acetate (34 mg, 0.15 mmol), potassium fluoride (174 mg, 3.0 mmol), 1,2,3,4,5-pentaphenyl-1′-(di-tert-butylphosphino) ferrocene (213 mg, 0.3 mmol) and diacetoxypalladium (70 mg, 0.10 mmol) in toluene (10 mL) and water (2 mL) was stirred at 110° C. under nitrogen atmosphere for 3 h. The reaction mixture was then concentrated and the residue was purified by flash chromatography on silica gel (10% ethyl acetate in petroleum ether) and further by prep-HPLC (Column Xbridge 21.2*250 mm C18, 10 um, Mobile Phase A: water (10 mmol/L ammonium bicarbonate) B: acetonitrile) to afford 6-chloro-3-methyl-4-phenylpyridazine (160 mg, 26%) as white solid. LCMS (ESI) m/z: 204.9/206.9 [M+H]⁺.

### Step 2: Synthesis of 4-(9-ethyl-2-(6-methyl-5-phenylpyridazin-3-yl)-8-(pyridin-4-yl)-9H-purin-6-yl)morpholine

A mixture of 4-(9-ethyl-8-(pyridin-4-yl)-2-(trimethylstannyl)-9H-purin-6-yl)morpholine (474 mg, 1.0 mmol), 6-chloro-3-methyl-4-phenylpyridazine (102 mg, 0.5 mmol) and bis(tri-tert-butylphosphine)palladium (10 mg, 0.02 mmol) in dioxane (5 mL) was stirred at 100° C. under nitrogen atmosphere for 16 h. The resultant mixture was concentrated and crude product was purified by silica gel column chromatography (20% dichloromethane in methanol) to afford 200 mg of a brown oil, which was further purified by prep-HPLC (Column Xbridge 21.2*250 mm C18, 10 um, Mobile Phase A: water (10 mmol/L ammonium bicarbonate) B: acetonitrile) to obtain 4-(9-ethyl-2-(6-methyl-5-phenylpyridazin-3-yl)-8-(pyridin-4-yl)-9H-purin-6-yl)morpholine (36.9 mg, 15.4%) as off-white solid.

¹H NMR (400 MHz, CDCl₃) δ 8.82 (dd, J=4.5, 1.5 Hz, 2H), 8.33 (s, 1H), 7.73 (dd, J=4.5, 1.6 Hz, 2H), 7.57-7.49 (m, 3H), 7.48-7.43 (m, 2H), 4.56 (q, J=7.2 Hz, 2H), 4.45 (bs, 4H), 3.92-3.86 (m, 4H), 2.77 (s, 3H), 1.48 (t, J=7.2 Hz, 3H); LCMS (ESI) m/z: 478.8 [M]⁺.

### Synthesis of 4-(9-ethyl-2-(5-phenylpyridazin-3-yl)-8-(pyridin-4-yl)-9H-purin-6-yl)morpholine (Compound 27)

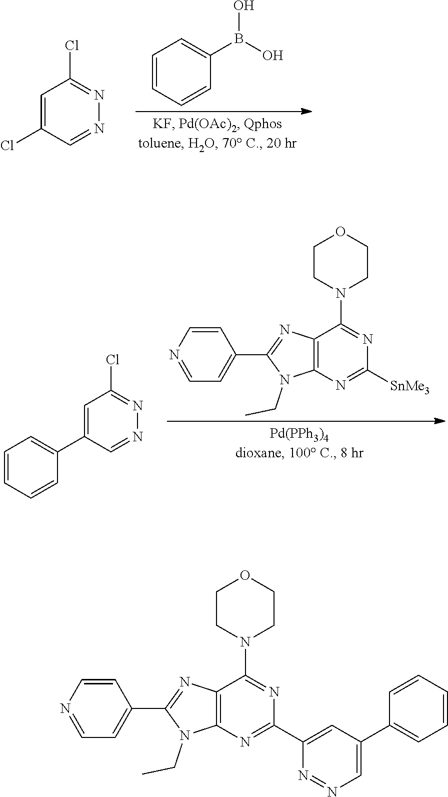

### Step 1: Preparation of 3-chloro-5-phenylpyridazine

To a solution of 3,5-dichloropyridazine (600 mg, 1.0 eq.) in toluene (10 mL) and water (5 mL) were added phenylboronic acid (589 mg, 1.2 eq.), potassium fluoride (467 mg, 8.054 mmol, 2.0 eq.), 1,2,3,4,5-pentaphenyl-1′-(di-tert-butylphosphino)ferrocene (70 mg, 0.10 mmol) and diacetoxypalladium (70 mg, 0.10 mmol). The mixture was stirred at 110° C. for 2 h and concentrated. The residue was purified by flash chromatography on silica gel (petroleum ether:ethyl acetate=5:95) to give the product as white solid (450 mg, 74.2%).

### Step 2: Preparation of 4-(9-ethyl-2-(5-phenylpyridazin-3-yl)-8-(pyridin-4-yl)-9H-purin-6-yl)morpholine

To a solution of 4-(9-ethyl-8-(pyridin-4-yl)-2-(trimethylstannyl)-9H-purin-6-yl)morpholine (300 mg) in dioxane (10 mL) were added 3-chloro-5-phenylpyridazine (400 mg, 1.0 eq) and tetrakis(triphenylphosphine)palladium (58 mg, 0.05 mmol). The mixture was stirred at 100° C. for 16 h and concentrated. The crude product was purified by Prep-HPLC (BOSTON pHlex ODS 10 um 21.2×250 mm 120 A. The mobile phase was acetonitrile/0.1% Ammonium bicarbonate) to afford 4-(9-ethyl-2-(5-phenylpyridazin-3-yl)-8-(pyridin-4-yl)-9H-purin-6-yl)morpholine (4 mg, 2.3%) as white solid.

¹H NMR (400 MHz, DMSO-d₆) δ 9.73 (d, J=2.2 Hz, 1H), 8.83 (s, 2H), 8.70 (d, J=2.1 Hz, 1H), 8.01 (d, J=6.8 Hz, 2H), 7.90 (d, J=5.0 Hz, 2H), 7.69-7.53 (m, 3H), 4.53 (d, J=7.0 Hz, 2H), 4.39 (s, 4H), 3.88-3.73 (m, 4H), 1.38 (t, J=7.1 Hz, 3H). LCMS (ESI) m/z: 464.9[M+H]⁺.

### Synthesis of 4-(9-ethyl-2-(6-phenylpyridazin-4-yl)-8-(pyridin-4-yl)-9H-purin-6-yl)morpholine (Compound 28)

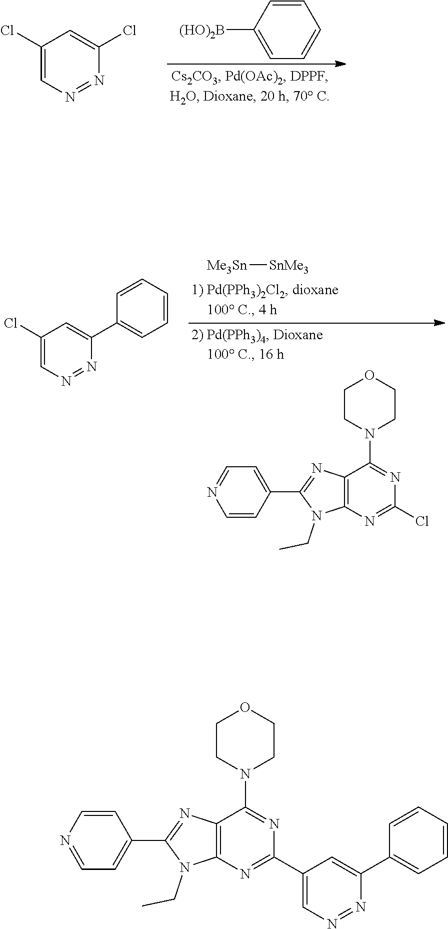

### Step 1: Synthesis of 5-chloro-3-phenylpyridazine

A solution 3,5-dichloropyridazine (600 mg, 4 mmol), phenylboronic acid (488 mg, 4 mmol), palladium (II) acetate (90 mg, 0.4 mmol), 1,1′-bis(diphenylphosphino)ferrocene (222 mg, 0.4 mmol) and cesium carbonate (3.91 g, 12 mmol) in water (3 mL) and dioxane (30 mL) was stirred at 70° C. for 20 h under argon. The resultant mixture was concentrated and purified by flash chromatography (dichloromethane/methanol=20:1) to get 5-chloro-3-phenylpyridazine (450 mg, 47%) as a white solid. LCMS: (ESI) m/z: 190.9 [M+H]⁺.

### Step 2: Synthesis of 4-(9-ethyl-2-(6-phenylpyridazin-4-yl)-8-(pyridin-4-yl)-9H-purin-6-yl)morpholine

To a solution of 5-chloro-3-phenylpyridazine (38 mg, 0.2 mmol) and bis(triphenylphosphine)palladiuM(II) chloride (28 mg, 0.04 mmol) in dioxane (10 mL) was added hexamethyldistannane (157 mg, 0.48 mmol), and the mixture was stirred at 100° C. for 4 h then cooled to room temperature. Then 4-(2-chloro-9-ethyl-8-(pyridin-4-yl)-9H-purin-6-yl)morpholine (69 mg, 0.2 mmol) and tetrakis(triphenylphosphine) palladium (46 mg, 0.04 mmol) were added to the reaction mixture and stirring was continued at 100° C. for anothe R¹6 h. The reaction mixture was concentrated and the crude residue was purified by Prep-HPLC (BOSTON pHlex ODS 10 um 21.2×250 mm 120 A. The mobile phase was acetonitrile/0.1% Ammonium bicarbonate) to afford 4-(9-ethyl-2-(6-phenylpyridazin-4-yl)-8-(pyridin-4-yl)-9H-purin-6-yl)morpholine (49.6 mg, 36%) as yellow solid.

1H NMR (400 MHz, DMSO-d6) δ 10.05 (s, 1H), 8.88-8.74 (m, 3H), 8.23 (d, J=7.6 Hz, 2H), 7.87 (d, J=4.8 Hz, 2H), 7.65-7.56 (m, 3H), 4.54 (q, J=7.2 Hz, 2H), 4.39 (bs, 4H), 3.86-3.76 (m, 4H), 1.40 (t, J=7.2 Hz, 3H); LCMS: (ESI) m/z 464.8 [M+]⁺.

### Synthesis of 2-methoxy-4-(9-methyl-6-morpholino-8-(pyridin-4-yl)-9H-purin-2-yl)phenol (Compound 29)

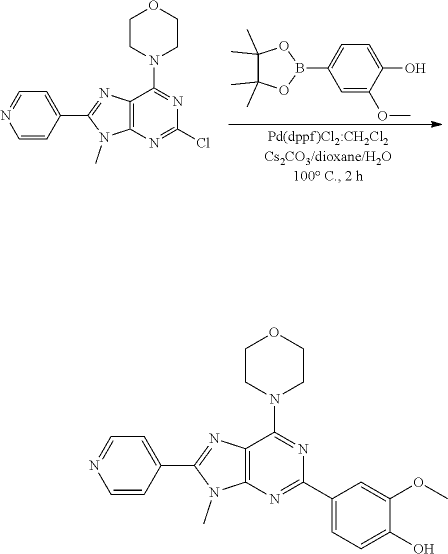

A mixture of 4-(2-chloro-9-methyl-8-(pyridin-4-yl)-9H-purin-6-yl)morpholine (165 mg, 0.5 mmol), 2-methoxy-4-(4,4,5,5-tetramethyl-1,3,2-dioxaborolan-2-yl)phenol (150 mg, 0.6 mmol), 1,1′-bis(diphenylphosphino)ferrocene-palladium(II)dichloride dichloromethane complex (41 mg, 0.05 mmol), and cesium carbonate (325 mg, 1.0 mmol) in water (1 mL) and dioxane (10 mL) was stirred at 100° C. under nitrogen atmosphere for 2 h. The mixture was concentrated and the residue was purified by silica gel column chromatography (20% dichloromethane in methanol) and further washed with methanol (15 mL) to afford 2-methoxy-4-(9-methyl-6-morpholino-8-(pyridin-4-yl)-9H-purin-2-yl)phenol (116.0 mg, 0.28 mmol, 56%) as a grey solid.

¹H NMR (400 MHz, DMSO-d₆) δ 9.41 (s, 1H), 8.79 (s, 2H), 8.01 (d, J=14.8 Hz, 1H), 7.96-7.88 (i, 3H), 6.87 (d, J=8.3 Hz, 1H), 4.33 (s, 4H), 3.98 (s, 3H), 3.88 (s, 3H), 3.82-3.75 (m, 4H); LCMS (ESI) m/z: 418.8 [M+H]⁺.

The following compounds were synthesized according to the protocol described above:

| Name | Structure | NMR, MS | # |
|---|---|---|---|
| 2-methoxy-5-(9- methyl-6- morpholino-8- (pyridin-4-yl)-9H- purin-2-yl)phenol | 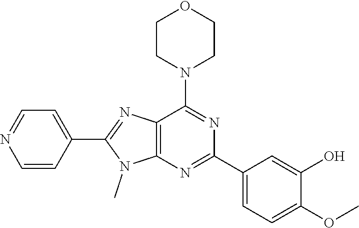 [SMILES: COc1ccc(-c2nc(N3CCOCC3)c3nc(-c4ccncc4)n(C)c3n2)cc1O] | 1H NMR (400 MHz, DMSO-d₆) δ 9.09 (s, 1H), 8.78 (d, J = 4.9 Hz, 2H), 7.92 (d, J = 1.7 Hz, 2H), 7.91 (s, 1H), 7.00 (d, J = 8.4 Hz, 1H), 5.76 (s, 1H), 4.33 (s, 4H), 3.99 (d, J = 14.4 Hz, 3H), 3.83 (s, 3H), 3.82-3.75 (m, 4H); LCMS (ESI) m/z: 419.7 [M + H]+. | 30 |
| 4-(2-(3,4- dimethoxyphenyl)- 9-methyl-8- (pyridin-4-yl)-9H- purin-6- yl)morpholine | 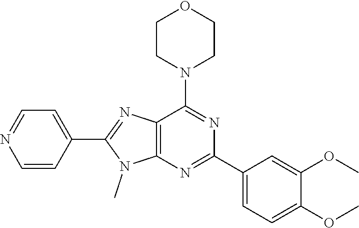 [SMILES: COc1ccc(-c2nc(N3CCOCC3)c3nc(-c4ccncc4)n(C)c3n2)cc1OC] | ¹H NMR (400 MHz, DMSO-d₆) δ 8.78 (d, J = 6.0 Hz, 2H), 8.06-8.03 (m, 2H), 7.92 (d, J = 8.4 Hz, 1H), 4.34-4.33 (bs, 4H), 4.00(s, 3H), 3.87 (s, 3H), 3.83 (s, 3H), 3.81-3.79 (m, 4H); LCMS (ESI) m/z: 433.1 [M + H]⁺. | 31 |
| 4-(9-cyclopropyl- 2-(3,4- dimethoxyphenyl)- 8-(pyridin-4-yl)- 9H-purin-6- yl)morpholine | 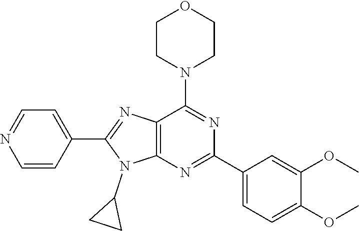 [SMILES: COc1ccc(-c2nc(N3CCOCC3)c3nc(-c4ccncc4)n(C4CC4)c3n2)cc1OC] | 1HNMR (400 MHz, DMSO-d₆) δ 8.77 (d, J = 5.5 Hz, 2H), 8.03 (t, J = 8.1 Hz, 4H), 7.07 (d, J = 8.4 Hz, 1H), 4.33 (bs, 4H), 3.94-3.69 (m, 11H), 1.18 (d, J = 6.1 Hz, 2H), 0.89 (s, 2H); LCMS: [M + H]⁺ = 458.8. | 32 |
| 4-(9-ethyl-2-(5- methoxypyridin-3- yl)-8-(pyridin-4- yl)-9H-purin-6- yl)morpholine | 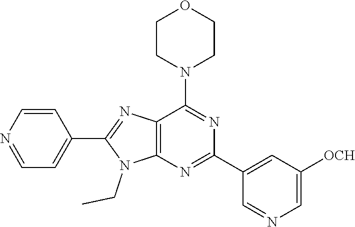 | ¹H NMR (400 MHz, DMSO-d₆) δ 9.19 (d, J = 1.6 Hz, 1H), 8.81 (dd, J = 4.5, 1.6 Hz, 2H), 8.40 (d, J = 2.9 Hz, 1H), 8.20 (dd, J = 2.9, 1.7 Hz, 1H), 7.86 (dd, J = 4.5, 1.6 Hz, 2H), 4.49 (q, J = 7.2 Hz, 2H), 4.35 (bs, 4H), 3.94 (s, 3H), 3.83-3.77 (m, 4H), 1.38 (t, J = 7.2 Hz, 3H). LCMS (ESI) m/z: 418.0. [M + H]+. | 33 |
| 4-(2-(3,4- dimethoxyphenyl)- 9-ethyl-8- (pyridin-4-yl)-9H- purin-6-yl)-3- methylmorpholine | 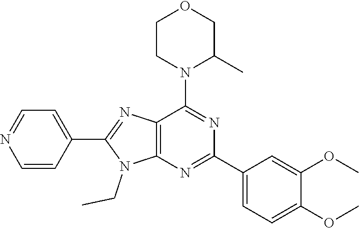 [SMILES: CCn1c(-c2ccncc2)nc2c(N3CCOCC3C)nc(-c3ccc(OC)c(OC)c3)nc21] | 1H NMR (400 MHz, DMSO-d₆) δ 8.80 (d, J = 5.4 Hz, 2H), 8.06-7.98 (m, 2H), 7.85 (d, J = 6.0 Hz, 2H), 7.07 (d, J = 8.5 Hz, 1H), 5.77-5.03 (m, 1H), 4.47 (q, J = 7.3 Hz, 2H), 4.03 (d, J = 8.7 Hz, 1H), 3.87 (s, 3H), 3.83 (d, J = 4.2 Hz, 4H), 3.78- 3.47 (m, 4H), 1.39-.1.36 (m, 6H); LCMS (ESI) m/z: 461.0 [M + H]⁺ | 34 |
| 4-(2-(3- cyclopropoxy- phenyl)-9- cyclopropyl-8- (pyridin-4-yl)-9H- purin-6- yl)morpholine | 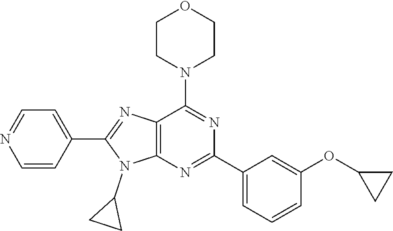 [SMILES: c1cc(OC2CC2)cc(-c2nc(N3CCOCC3)c3nc(-c4ccncc4)n(C4CC4)c3n2)c1] | 1H NMR (400 MHz, DMSO-d₆) δ 8.78 (d, J = 5.8 Hz, 2H), 8.12 (s, 1H), 8.04 (dd, J = 13.9, 6.9 Hz, 3H), 7.42 (t, J = 7.9 Hz, 1H), 7.18 (d, J = 6.1 Hz, 1H), 4.34 (bs, 4H), 3.92 (s, 1H), 3.79 (s, 5H), 1.18 (d, J = 6.1 Hz, 2H), 0.94-0.79 (m, 4H), 0.72 (s, 2H); LC-MS: [M + H]⁺ = 454.8 | 35 |
| 4-(2-(3,4- dimethoxyphenyl)- 9-ethyl-8- (pyridin-4-yl)-9H- purin-6- yl)morpholine | 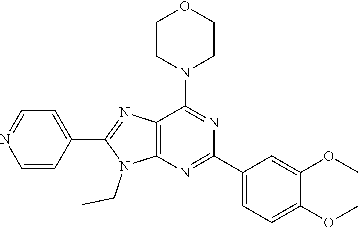 [SMILES: CCn1c(-c2ccncc2)nc2c(N3CCOCC3)nc(-c3ccc(OC)c(OC)c3)nc21] | 1H NMR (500 MHz, DMSO-d₆) δ 8.80 (d, J = 5.4 Hz, 2H), 8.03 (d, J = 4 Hz, 2H), 8.01(s, 1H), 7.85 (d, J = 5.8 Hz, 2H), 7.07 (d, J = 8.5 Hz, 1H), 4.47 (q, J = 7.1 Hz, 2H), 4.34 (bs, 4H), 3.87 (s, 3H), 3.83 (s, 3H), 3.81-3.76 (m, 4H), 1.37 (t, J = 7.1 Hz, 3H); LCMS (ESI) m/z: 447.2 [M + H]⁺ | 36 |
| 4-(9-ethyl-2-(5- methoxypyridin-3- yl)-8-(pyridin-4- yl)-9H-purin-6-yl)- 3- methylmorpholine | 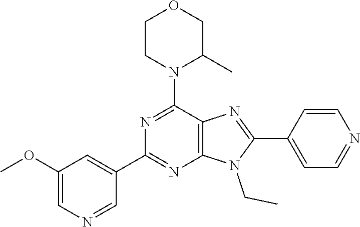 [SMILES: CCn1c(-c2ccncc2)nc2c(N3CCOCC3C)nc(-c3cncc(OC)c3)nc21] | 1H NMR (400 MHz, DMSO-d₆) δ 9.19 (s, 1H), 8.80 (s, 2H), 8.40 (s, 1H), 8.19 (s, 1H), 7.85 (s, 2H), 5.32 (bs, 2H), 4.47 (s, 2H), 4.02-3.75 (m, 6H), 3.56 (s, 2H), 1.37 (s, 6H); LCMS (ESI) m/z: 432.1 [M + H]⁺ | 37 |
| 4-(2-(3- cyclopropoxy- phenyl)-9-ethyl-8- (pyridin-4-yl)-9H- purin-6-yl)-3- methylmorpholine | 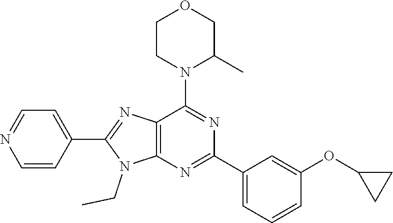 [SMILES: CCn1c(-c2ccncc2)nc2c(N3CCOCC3C)nc(-c3cccc(OC4CC4)c3)nc21] | 1H NMR (400 MHz, DMSO-d₆) δ 8.80 (d, J = 5.6 Hz, 2H), 8.10 (s, 1H), 8.04 (d, J = 7.8 Hz, 1H), 7.85 (d, J = 5.9 Hz, 2H), 7.42 (t, J = 7.9 Hz, 1H), 7.18 (dd, J = 8.0, 2.0 Hz, 1H), 5.59-5.13 (m, 1H), 4.46 (q, J = 7.2 Hz, 2H), 4.03 (d, J = 8.4 Hz, 1H), 3.97-3.88 (m, 1H), 3.83 (d, J = 11.5 Hz, 1H), 3.74 (d, J = 11.6 Hz, 1H), 3.65-3.43 (m, 2H), 1.39 (t, J = 7.3 Hz, 6H), 0.83 (d, J = 5.9 Hz, 2H), 0.72 (s, 2H); LCMS: (ESI) m/z: 456.9 [M + H]⁺. | 38 |
| 4-(2-(2- cyclopropoxy- phenyl)-9-ethyl-8- (pyridin-4-yl)-9H- purin-6- yl)morpholine | 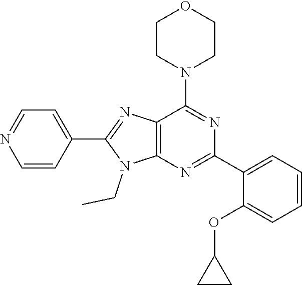 [SMILES: CCn1c(-c2ccncc2)nc2c(N3CCOCC3)nc(-c3ccccc3OC3CC3)nc21] | ¹H NMR (400 MHz, DMSO-d₆) δ 8.80 (d, J = 5.2 Hz, 2H), 7.83 (d, J = 5.5 Hz, 2H), 7.66 (d, J = 7.2 Hz, 1H), 7.42 (d, J = 3.0 Hz, 2H), 7.11-7.00 (m, 1H), 4.48-4.09 (m, 6H), 3.91 (s, 1H), 3.75 (s, 4H), 1.35 (t, J = 7.0 Hz, 3H), 0.77 (d, J = 5.4 Hz, 2H), 0.64 (s, 2H); LCMS (ESI) m/z: 442.8[M + H]+. | 39 |
| 4-(2-(3- cyclopropoxy- phenyl)-9-ethyl-8- (pyridin-4-yl)-9H- purin-6- yl)morpholine | 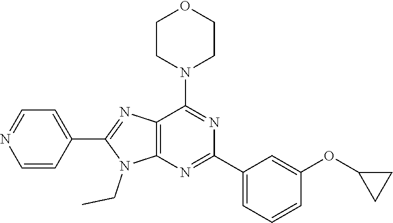 [SMILES: CCn1c(-c2ccncc2)nc2c(N3CCOCC3)nc(-c3cccc(OC4CC4)c3)nc21] | 1H NMR (400 MHz, DMSO-d₆) δ 8.80 (dd, J = 4.5, 1.5 Hz, 2H), 8.09 (dd, J = 2.4, 1.5 Hz, 1H), 8.07-8.02 (m, 1H), 7.85 (dd, J = 4.5, 1.6 Hz, 2H), 7.42 (t, J = 7.9 Hz, 1H), 7.19 (dd, J = 8.1, 1.7 Hz, 1H), 4.46 (q, J = 7.2 Hz, 2H), 4.44 (bs, 4H), 3.93 (pent, 3.2 Hz, 1H), 3.83-3.75 (m, 4H), 1.38 (t, J = 7.2 Hz, 3H), 0.83 (q, J = 5.9 Hz, 2H), 0.77-0.66 (m, 2H); LCMS: (ESI) m/z: 442.8 [M + H]⁺. | 40 |
| 4-(2-(3-(1H- pyrazol-1- yl)phenyl)-9-ethyl- 8-(pyridin-4-yl)- 9H-purin-6- yl)morpholine | 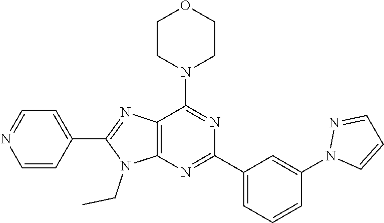 [SMILES: CCn1c(-c2ccncc2)nc2c(N3CCOCC3)nc(-c3cccc(-n4cccn4)c3)nc21] | 1H NMR (400 MHz, DMSO-d₆) δ 8.85-8.83 (m, 1H), 8.81 (dd, J = 4.5, 1.5 Hz, 2H), 8.60 (d, J = 2.4 Hz, 1H), 8.38 (d, J = 7.9 Hz, 1H), 7.93 (dd, J = 8.0, 1.4 Hz, 1H), 7.86 (dd, J = 4.5, 1.6 Hz, 2H), 7.82 (d, J = 1.5 Hz, 1H), 7.62 (t, J = 7.9 Hz, 1H), 6.62-6.58 (m, 1H), 4.50 (q, J = 7.2 Hz, 2H), 4.37 (bs, 4H), 3.86-3.77 (m, 4H), 1.39 (t, J = 7.2 Hz, 3H); LCMS: (ESI) m/z: 453.3 [M + H]⁺. | 41 |
| 4-(9-ethyl-2-(3-(1- methyl-1H- pyrazol-3- yl)phenyl)-8- (pyridin-4-yl)-9H- purin-6-yl)-3- methylmorpholine | 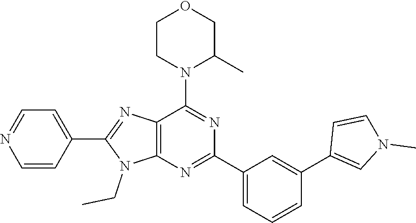 [SMILES: CCn1c(-c2ccncc2)nc2c(N3CCOCC3C)nc(-c3cccc(-c4ccn(C)c4)c3)nc21] | ¹H NMR (400 MHz, DMSO-d₆) δ 8.81 (d, J = 5.7 Hz, 3H), 8.35 (d, J = 7.8 Hz, 1H), 7.89-7.85 (m, 3H), 7.79 (s, 1H), 7.52 (t, J = 7.7 Hz, 1H), 6.76 (d, J = 2.2 Hz, 1H), 5.77-5.39 (m, 1H), 5.39- 4.94 (m, 1H), 4.50 (q, J = 7.0 Hz, 2H), 4.09- 3.72 (m, 6H), 3.65-3.40 (m, 2H), 1.47-1.33 (m, 6H); | 42 |

### Synthesis of 4-[9-ethyl-2-(1H-indazol-4-yl)-8-(4-pyridyl)purin-6-yl]morpholine (Compound 43)

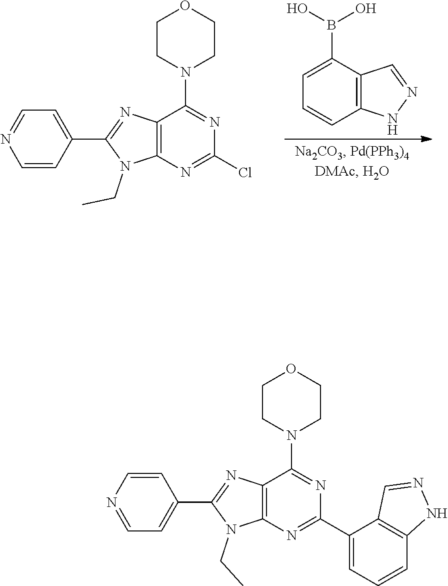

To a mixture of 4-[2-chloro-9-ethyl-8-(4-pyridyl)purin-6-yl]morpholine (150 mg, 435 umol) in DMAc (2 mL) were added 1H-indazol-4-ylboronic acid (106 mg, 653 umol), Na₂O₃ (1 M in water, 1.31 m), Pd(PPh₃)₄ (50 mg, 44 umol) under nitrogen atmosphere and the resultant mixture was heated at 120° C. for 30 min. under microwave irradiation. After the aqueous work up and extraction with ethyl acetate, the resultant crude product was purified by prep-HPLC (Phenomenex luna C18 80*40 mm*3 umcolumn; 25-43% acetonitrile in an a 0.04% HCl solution in water, 7 min gradient) to obtain 4-[9-ethyl-2-(1H-indazol-4-yl)-8-(4-pyridyl)purin-6-yl]morpholine (85 mg, 46%) as yellow solid.

¹H NMR (400 MHz, METHANOL-d₄) δ 9.05 (s, 1H), 8.97 (d, J=6.9 Hz, 2H), 8.60 (d, J=6.8 Hz, 2H), 8.37 (d, J=7.1 Hz, 1H), 7.71 (d, J=8.3 Hz, 1H), 7.62-7.48 (i, 1H), 4.80-4.76 (i, 2H), 4.51 (bs, 4H), 3.98-3.84 (i, 4H), 13.65 (t, J=7.2 Hz, 3H). LCMS (ESI for C23H22N8O) [M+H]⁺: 427.1.

The following compounds were synthesized according to the protocol described above:

| Name | Structure | NMR, MS | # |
|---|---|---|---|
| 4-(9-methyl-2- (pyridin-3-yl)-8- (pyridin-4-yl)-9H- purin-6- yl)morpholine | 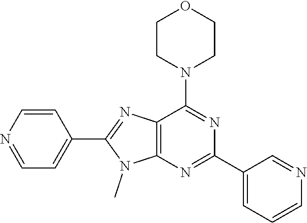 [SMILES: Cn1c(-c2ccncc2)nc2c(N3CCOCC3)nc(-c3cccnc3)nc21] | ¹H NMR (400 MHz, DMSO-d₆) δ 3¹H NMR (400 MHz, DMSO-d₆) δ 9.58(d, J = 2.0 Hz, 1H), 8.79(d, J = 6.0 Hz, 2H), 8.72(d, J = 8.0 Hz, 1H), 8.66-8.67(m, 1H), 7.93(d, J = 6.0 Hz, 2H), 7.53(dd, J = 8.0, 4.8 Hz, 1H), 4.00(s, 3H), 4.37(s, 4H), 3.80(t, J = 4.4 Hz, 4H); LCMS (ESI) m/z: 374.3 [M + H]⁺. | 44 |
| 4-(2-(2,3- dihydrobenzo[b] [1,4]dioxin-5-yl)-9- methyl-8-(pyridin- 4-yl)-9H-purin-6- yl)morpholine | 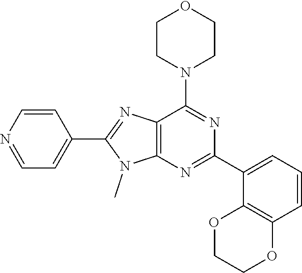 [SMILES: Cn1c(-c2ccncc2)nc2c(N3CCOCC3)nc(-c3cccc4c3OCCO4)nc21] | ¹H NMR (400 MHz, Chloroform-d) δ 8.86-8.75 (m, 2H), 7.79-7.72 (m, 2H), 7.41-7.35 (m, 1H), 7.00- 6.86 (m, 2H), 4.65-4.10 (m, 8H), 3.99 (s, 3H), 3.93- 3.78 (m, 4H); LCMS (ESI) m/z: 431.3 [M + H]⁺. | 45 |
| 4-(2-(2,3- dihydrobenzo[b] [1,4]dioxin-6-yl)-9- methyl-8-(pyridin- 4-yl)-9H-purin-6- yl)morpholine | 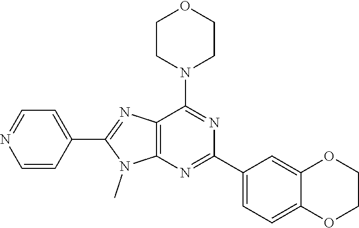 [SMILES: Cn1c(-c2ccncc2)nc2c(N3CCOCC3)nc(-c3ccc4c(c3)OCCO4)nc21] | ¹H NMR (500 MHz, Chloroform-d) δ 8.81-8.76 (m, 2H), 8.06-7.98 (m, 2H), 7.79-7.72 (m, 2H), 6.94 (d, J = 8.4 Hz, 1H), 4.57-4.21 (m, 8H), 4.00 (s, 3H), 3.93-3.84 (m, 4H); LCMS (ESI) m/z: 431.1 [M + H]⁺. | 46 |
| 4-(2-(1- cyclopropyl-1H- indazol-4-yl)-9- ethyl-8-(pyridin-4- yl)-9H-purin-6- yl)morpholine | 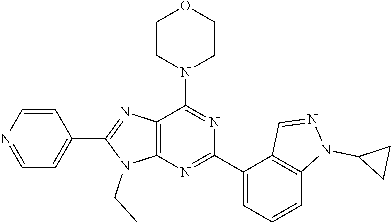 [SMILES: CCn1c(-c2ccncc2)nc2c(N3CCOCC3)nc(-c3cccc4c3cnn4C3CC3)nc21] | ¹H NMR (400 MHz, CHLOROFORM-d) δ = 8.95 (s, 1H), 8.82 (d, J = 5.6 Hz, 2H), 8.34 (d, J = 7.2 Hz, 1H), 7.83 (bs, 2H), 7.74 (d, J = 8.4 Hz, 1H), 7.52 (t, J = 7.8 Hz, 1H), 4.58-4.48 (m, 6H), 3.93-3.91 (m, 4H), 3.68-3.65 (m, 1H), 1.61 (t, J = 7.2 Hz, 3H), 1.29-1.25 (m, 2H), 1.23-1.18 (m, 2H). LCMS (ESI) for (C26H26N8O) [M + H]⁺: 467.2 | 47 |
| 4-(2- (benzo[d] [1,3]dioxol- 5-yl)-9-methyl- 8-(pyridin-4-yl)- 9H-purin-6- yl)morpholine | 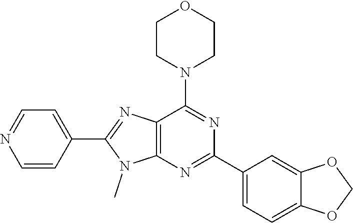 [SMILES: Cn1c(-c2ccncc2)nc2c(N3CCOCC3)nc(-c3ccc4c(c3)OCO4)nc21] | ¹H NMR (400 MHz, DMSO-d₆) δ 8.78 (d, J = 5.5 Hz, 2H), 8.04 (d, J = 8.2 Hz, 1H), 7.91 (d, J = 5.7 Hz, 3H), 7.01 (d, J = 8.2 Hz, 1H), 6.10 (s, 2H), 4.33 (s, 4H), 3.97 (s, 3H), 3.79 (s, 4H); LCMS (ESI) m/z: 417.1 [M + H]⁺. | 48 |
| 4-(2-(5- chloropyridin-2- yl)-9-methyl-8- (pyridin-4-yl)-9H- purin-6- yl)morpholine | 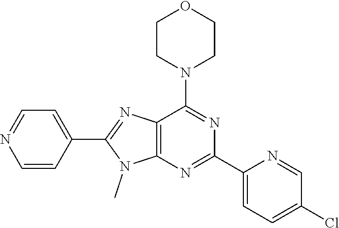 [SMILES: Cn1c(-c2ccncc2)nc2c(N3CCOCC3)nc(-c3ccc(Cl)cn3)nc21] | ¹H NMR (400 MHz, DMSO-d₆) δ 8.81-8.77 (m, 3H), 8.49 (d, J = 8.8 Hz, 1H), 8.08-8.06 (m, 1H), 7.93(d, J = 6.0 Hz, 1H), 4.35 (bs, 4H), 3.99 (s, 3H), 3.81-3.78 (m, 4H); LCMS (ESI) m/z: 408.1 [M + H]⁺. | 49 |
| 4-(2-cyclopropyl- 9-methyl-8- (pyridin-4-yl)-9H- purin-6- yl)morpholine | 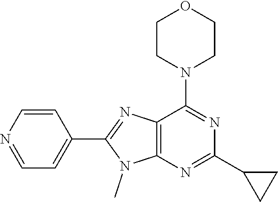 [SMILES: Cn1c(-c2ccncc2)nc2c(N3CCOCC3)nc(C3CC3)nc21] | ¹H NMR (400 MHz, DMSO-d₆) δ 8.76 (d, J = 5.1 Hz, 2H), 7.87 (d, J = 5.3 Hz, 2H), 4.21 (bs, 4H), 3.86 (s, 3H), 3.72 (t, J = 4.8 Hz, 4H), 2.05 (s, 1H), 1.07-0.81 (m, 4H); LCMS (ESI) m/z: 337.2 [M + H]⁺. | 50 |
| 4-(9-methyl-2- (pyridin-2-yl)-8- (pyridin-4-yl)-9H- purin-6- yl)morpholine | 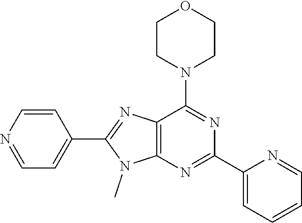 [SMILES: Cn1c(-c2ccncc2)nc2c(N3CCOCC3)nc(-c3ccccn3)nc21] | ¹HNMR (400 MHz, DMSO-d₆) δ ¹HNMR (400 MHz, DMSO-d₆) δ 8.79 (dd, J = 4.4, 1.6 Hz, 2H), 8.73(d, J = 3.6 Hz, 1H), 8.44 (d, J = 8.0 Hz, 1H), 7.91-7.96(m, 3H), 7.46-7.49(m, 1H), 4.36(s, 4H), 4.00(s, 3H), 3.79(t, J = 4.8 Hz, 4H), LCMS (ESI) m/z: 374.3 [M + H]⁺. | 51 |

### Synthesis of 2-methyl-6-(9-methyl-6-morpholino-8-(pyridin-4-yl)-9H-purin-2-yl)isoindolin-1-one (Compound 52)

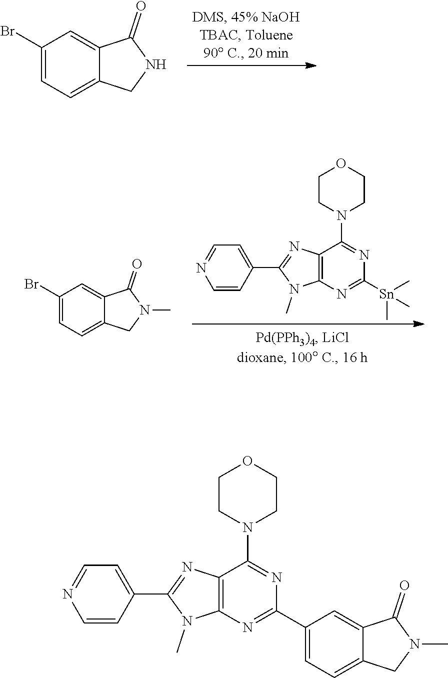 [SMILES: O=C1NCc2ccc(Br)cc21 ; CN1Cc2ccc(Br)cc2C1=O ; Cn1c(-c2ccncc2)nc2c(N3CCOCC3)n[c]([Sn]([CH3])([CH3])[CH3])nc21 ; CN1Cc2ccc(-c3nc(N4CCOCC4)c4nc(-c5ccncc5)n(C)c4n3)cc2C1=O]

### Step 1: Synthesis of 6-bromo-2-methylisoindolin-1-one

A mixture of 6-bromoisoindolin-1-one (100 mg, 0.47 mmol), Me₂SO₄ (0.1 mL, 0.71 mmol), NaOH (aq. 45%) (419 mg, 4.72 mmol) and Bu₄NCl (26 mg, 0.09 mmol) in toluene (5 mL) was stirred at 80° C. for 12 min. The mixture was concentrated and purified by column chromatography (50% EA in PE) to give the desired compound as white solid (30 mg, 60%). LCMS (ESI) m/z: 226 [M+H]⁺.

### Step 2: Synthesis of 2-methyl-6-(9-methyl-6-morpholino-8-(pyridin-4-yl)-9H-purin-2-yl)isoindolin-1-one

To a solution of 4-(9-methyl-8-(pyridin-4-yl)-2-(trimethylstannyl)-9H-purin-6-yl)morpholine (80 mg, 0.17 mmol), 6-bromo-2-methylisoindolin-1-one (47 mg, 0.21 mmol) and LiCl (26 mg, 0.51 mmol) in dioxane (10 mL) was added Pd(PPh₃)₄ (25 mg, 0.02 mmol) and the resultant mixture was stirred at 100° C. for 16 h under nitrogen atmosphere. The mixture was then concentrated and purified by Prep-HPLC to obtain the desired product (6 mg, 10%) as yellow solid.

¹H NMR (400 MHz, DMSO-d6) δ 8.79 (d, J=5.2 Hz, 1H), 8.70-8.66 (m, 2H), 7.93 (d, J=5.6 Hz, 2H), 7.69 (d, J=8.0 Hz, 1H), 4.53 (s, 2H), 4.40-4.33 (m, 4H), 4.03 (s, 3H), 4.02-3.82 (m, 4H), 3.11 (s, 3H); LCMS (ESI) m/z: 442.2 [M+H]⁺.

### Synthesis of 4-(9-ethyl-2-(pyridazin-4-yl)-8-(pyridin-4-yl)-9H-purin-6-yl)morpholine (Compound 53)

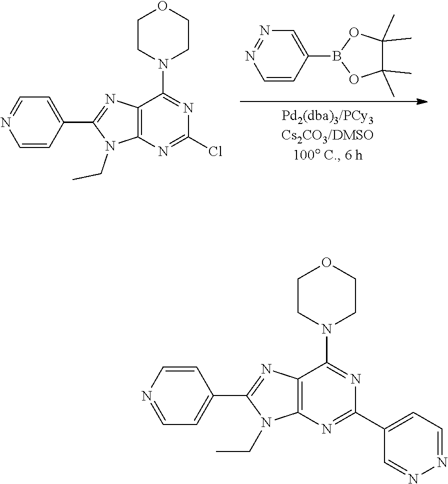 [SMILES: CCn1c(-c2ccncc2)nc2c(N3CCOCC3)nc(Cl)nc21 ; CC1(C)OB(c2ccnnc2)OC1(C)C ; CCn1c(-c2ccncc2)nc2c(N3CCOCC3)nc(-c3ccnnc3)nc21]

A mixture of 4-(2-chloro-9-ethyl-8-(pyridin-4-yl)-9H-purin-6-yl)morpholine (86 mg, 0.25 mmol), 4-(4,4,5,5-tetramethyl-1,3,2-dioxaborolan-2-yl)pyridazine (60 mg, 0.3 mmol), tris(dibenzylideneacetone)dipalladium(0) (23 mg, 0.025 mmol), tricyclohexylphosphine (14 mg, 0.05 mmol) and cesium carbonate (163 mg, 0.5 mmol) in dimethyl sulfoxide (4 mL) was stirred at 100° C. under nitrogen atmosphere for 6 h. The mixture was purified by prep-HPLC (Column Xbridge 21.2*250 mm C18, 10 um, Mobile Phase A: water (10 mmol/L ammonium bicarbonate) B: acetonitrile) to afford 4-(9-ethyl-2-(pyridazin-4-yl)-8-(pyridin-4-yl)-9H-purin-6-yl)morpholine (21.0 mg, 21.6) as a grey solid.

¹H NMR (400 MHz, CDCl₃) δ 10.17 (dd, J=2.1, 1.3 Hz, 1H), 9.32 (dd, J=5.3, 1.2 Hz, 1H), 8.83 (dd, J=4.5, 1.6 Hz, 2H), 8.42 (dd, J=5.3, 2.2 Hz, 1H), 7.71 (dd, J=4.5, 1.6 Hz, 2H), 4.50 (q, J=7.2 Hz, 6H), 3.95-3.84 (m, 4H), 1.55 (t, J=7.2 Hz, 3H); LCMS (ESI) m/z: 388.9 [M+H]⁺.

### Synthesis of 4-(9-methyl-8-(pyridin-4-yl)-2-(1,2,3,4-tetrahydroquinolin-7-yl)-9H-purin-6-yl)morpholine (Compound 54)

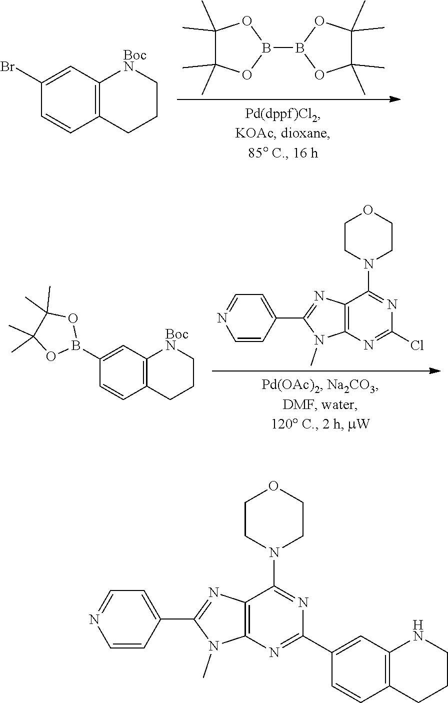

### Step 1: Preparation of tert-butyl 7-(4,4,5,5-tetramethyl-1,3,2-dioxaborolan-2-yl)-3,4-dihydroquinoline-1(2H)-carboxylate

To a solution of tert-butyl 7-bromo-3,4-dihydroquinoline-1(2H)-carboxylate (622 mg, 2 mmol) in dioxane (10 mL) was added 4,4,4′,4′,5,5,5′,5′-octamethyl-2,2′-bi(1,3,2-dioxaborolane) (765 mg, 3 mmol), [1,1′-bis(diphenylphosphino)ferrocene]dichloro palladium (II) (146 mg, 0.2 mmol) and potassium acetate (588 mg, 6 mmol) at 25° C. and the reaction mixture was stirred at 85° C. for 16 h under nitrogen protection. The mixture was extracted with ethyl acetate (20 mL*2) and washed with water (10 mL*2). The organic layer was dried over sodium sulfate, and concentrated. The crude product was purified by flash chromatography on silica gel (petroleum ether/ester acetic=10:1-3:1) to give tert-butyl 7-(4,4,5,5-tetramethyl-1,3,2-dioxaborolan-2-yl)-3,4-dihydroquinoline-1(2H)-carboxylate as a white solid. (610 mg, 84.9%). LCMS (ESI) m/z: 304.2 [M*-55]⁺.

### Step 2: Preparation of 4-(9-methyl-8-(pyridin-4-yl)-2-(1,2,3,4-tetrahydroquinolin-7-yl)-9H-purin-6-yl)morpholine

To a solution of 4-(2-chloro-9-methyl-8-(pyridin-4-yl)-9H-purin-6-yl)morpholine (300 mg, 0.83 mmol) in N,N-dimethylformamide (5 mL) was added tert-butyl 7-(4,4,5,5-tetramethyl-1,3,2-dioxaborolan-2-yl)-3,4-dihydroquinoline-1(2H)-carboxylate (132 mg, 0.4 mmol), palladium (II) acetate (20 mg, 0.08 mmol) and sodium carbonate (124 mg, 1.2 mmol) at 25° C. The sealed vial was stirred at 120° C. under microwave for 2 h. The mixture was extracted with ethyl acetate (20 mL*2) and washed with water (10 mL*2). The organic layer was dried over sodium sulfate, and concentrated. The residue (50 mg, 0.1 mmol) was mixed with dichloromethane (5 mL) and trifluoroacetic acid (2 mL), the mixture was stirred at room temperature for 1 h and concentrated. The residue was purified with prep-HPLC (BOSTON pHlex ODS 10 μm 21.2×250 mm 120 A. The mobile phase was dimethyl sulfoxide/0.1% Ammonium bicarbonate) to give 4-(9-methyl-8-(pyridin-4-yl)-2-(1,2,3,4-tetrahydroquinolin-7-yl)-9H-purin-6-yl)morpholine as white solid (17.3 mg, 13.3%).

¹H NMR (400 MHz, DMSO-d₆) δ 8.78 (d, J=5.6 Hz, 2H), 7.91 (d, J=5.6 Hz, 2H), 7.62-7.49 (m, 2H), 6.92 (d, J=7.8 Hz, 1H), 5.81 (s, 1H), 4.34 (s, 4H), 3.96 (s, 3H), 3.79 (s, 4H), 3.21 (s, 2H), 2.71 (t, J=5.9 Hz, 2H), 1.82 (s, 2H); LCMS (ESI) m/z: 428.0 [M+H]⁺.

### Synthesis of (5-(9-ethyl-6-(3-methylmorpholino)-8-(pyridin-4-yl)-9H-purin-2-yl)-2-methoxyphenyl)methanol (Compound 55)

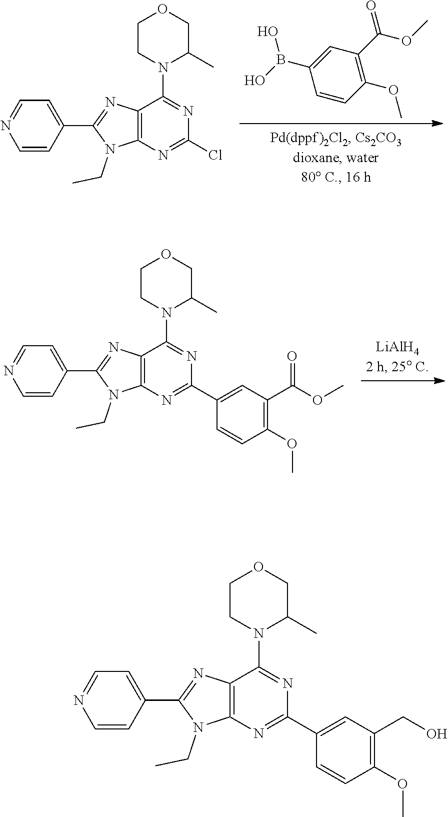 [SMILES: CCn1c(-c2ccncc2)nc2c(N3CCOCC3C)nc(Cl)nc21 ; COC(=O)c1cc(B(O)O)ccc1OC ; CCn1c(-c2ccncc2)nc2c(N3CCOCC3C)nc(-c3ccc(OC)c(C(=O)OC)c3)nc21 ; CCn1c(-c2ccncc2)nc2c(N3CCOCC3C)nc(-c3ccc(OC)c(CO)c3)nc21]

### Step 1: Synthesis of methyl 5-(9-ethyl-6-(3-methylmorpholino)-8-(pyridin-4-yl)-9H-purin-2-yl)-2-methoxybenzoate

A solution of 4-(2-chloro-9-ethyl-8-(pyridin-4-yl)-9H-purin-6-yl)-3-methylmorpholine (200 mg, 0.55 mmol), (4-methoxy-3-(methoxycarbonyl) phenyl)boronic acid (175 mg, 0.83 mmol), [1,1′-bis(diphenylphosphino)ferrocene]dichloropalladium(II) (40 mg, 0.055 mmol) and cesium carbonate (357 mg, 1.1 mmol) in dioxane (5 mL) and water (0.5 mL) was stirred at 80° C. for 16 h under nitrogen. Then water was added and the mixture was extracted with ethyl acetate (50 mL×3). The organic layer was dried and concentrated. The crude residue was purified by Pre-TLC (petroleum ether:ethyl acetate from 50:1 to 10:1) to give methyl 5-(9-ethyl-6-(3-methylmorpholino)-8-(pyridin-4-yl)-9H-purin-2-yl)-2-methoxybenzoate (250 mg, 92%) as a white solid. LCMS (ESI) m/z: 489.3 [M+H]⁺.

### Step 2: Synthesis of (5-(9-ethyl-6-(3-methylmorpholino)-8-(pyridin-4-yl)-9H-purin-2-yl)-2-methoxyphenyl)methanol

To a suspension of LiAlH4 (1 mol/L in tetrahydrofuran, 0.5 mol, 0.5 mL) in tetrahydrofuran (1.5 mL) at 0° C., was added a solution of methyl 5-(9-ethyl-6-(3-methylmorpholino)-8-(pyridin-4-yl)-9H-purin-2-yl)-2-methoxybenzoate (50 mg, 0.1 mol) in tetrahydrofuran (0.5 mL) was carefully. The reaction was allowed to warm to room temperature and stirred for 2 h. The mixture was cooled to 0° C., quenched with water (0.1 mL) and aqueous sodium hydroxide (1 N, 0.2 mL). The reaction was allowed to warm to room temperature and stirred for 1 h. Then dichloromethane (10 mL) were added and the salts were filtered. The filtrate was treated with brine (5 mL) and extracted with dichloromethane (20 mL×3). The organic layer was concentrated under reduced pressure and the residue was purified by column chromatography on silica gel (Dichloromethane/Methanol 20:1→10:1) to afford 9-ethyl-6-(3-methylmorpholino)-8-(pyridin-4-yl)-9H-purin-2-yl)-2-methoxyphenyl)methanol (29. mg, 66%) as a white solid.

1H NMR (400 MHz, DMSO-d₆) δ 8.80 (d, J=5.4 Hz, 2H), 8.48 (s, 1H), 8.32 (d, J=6.7 Hz, 1H), 7.85 (d, J=5.6 Hz, 2H), 7.05 (d, J=8.7 Hz, 1H), 5.5 (bs, 1H), 5.12 (bs, 2H), 4.56 (s, 2H), 4.46 (q, J=7.1 Hz, 2H), 4.04 (d, J=9.4 Hz, 1H), 3.95-3.70 (m, 4H), 3.74 (d, J=9.0 Hz, 1H), 3.63-3.46 (m, 2H), 1.40-1.25 (m, 6H); LCMS (ESI) m/z: 461.3 [M+H]⁺.

### Synthesis of 4-(2-(2,3-dihydrobenzo[b][1,4]dioxin-6-yl)-9-methyl-8-(piperidin-4-yl)-9H-purin-6-yl)morpholine (Compound 56)

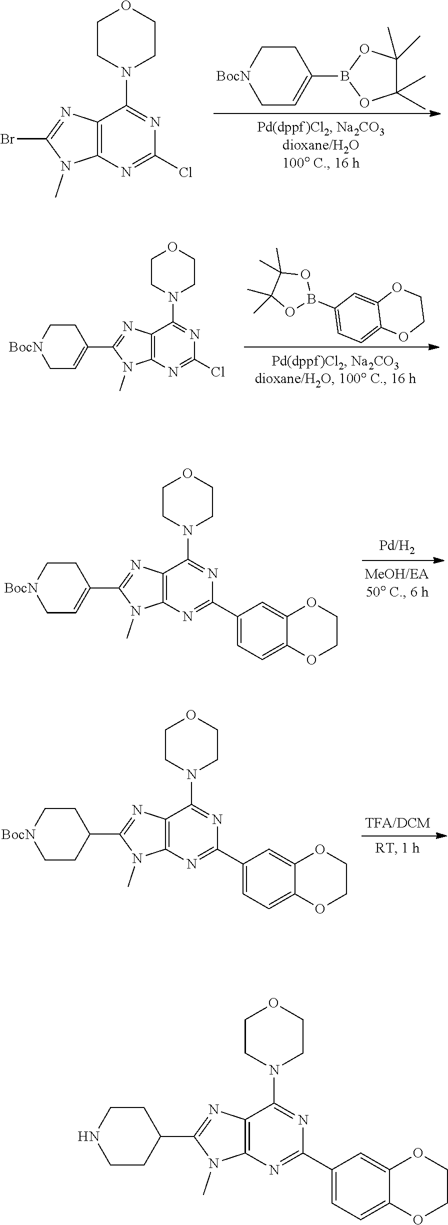

### Step 1: Synthesis of tert-butyl 4-(2-chloro-9-methyl-6-morpholino-9H-purin-8-yl)-3,6-dihydropyridine-1(2H)-carboxylate

A mixture of 4-(8-bromo-2-chloro-9-methyl-9H-purin-6-yl)morpholine (120 mg, 0.36 mmol), tert-but yl 4-(4,4,5,5-tetramethyl-1,3,2-dioxaborolan-2-yl)-3,6-dihydropyridine-1(2H)-carboxylate (112 mg, 0.36 mm ol), Na₂CO₃ (115 mg, 1.08 mmol) and Pd(dppf)Cl₂ (26 mg, 0.04 mmol) in dioxane (8 mL) and H₂O (1 mL) was stirred at 80° C. for 2 h under nitrogen atmosphere. The mixture was then concentrated and the crude pro duct was purified by column chromatography (30% EA in PE) to obtain the desired compound as white so lid (100 mg, 64%). LCMS (ESI) m/z: 435 [M+H]⁺.

### Step 2: Synthesis of tert-butyl 4-(2-(2,3-dihydrobenzo[b][1,4]dioxin-6-yl)-9-methyl-6-morpholino-9H-purin-8-yl)-3,6-dihydropyridine-1(2H)-carboxylate

A mixture of tert-butyl 4-(2-chloro-9-methyl-6-morpholino-9H-purin-8-yl)-3,6-dihydropyridine-1(2H)-carboxylate (100 mg, 0.23 mmol), 2-(2,3-dihydrobenzo[b][1,4]dioxin-6-yl)-4,4,5,5-tetramethyl-1,3,2-dioxaborolane (121 mg, 0.46 mmol), Na₂CO₃ (73 mg, 0.69 mmol) and Pd(dppf)Cl₂ (17 mg, 0.02 mmol) in dioxane (8 mL) and H₂O (1 mL) was stirred at 80° C. for 2 h under nitrogen atmosphere. The resultant mixture was concentrated and the crude product was purified by column chromatography (30% EA in PE) to obtain the desired product as white solid (80 mg, 65%). LCMS (ESI) m/z: 535 [M+H]⁺.

### Step 3: Synthesis of tert-butyl 4-(2-(2,3-dihydrobenzo[b][1,4]dioxin-6-yl)-9-methyl-6-morpholino-9H-purin-8-yl)piperidine-1-carboxylate

A suspension of tert-butyl 4-(2-(2,3-dihydrobenzo[b][1,4]dioxin-6-yl)-9-methyl-6-morpholino-9H-purin-8-yl)-3,6-dihydropyridine-1(2H)-carboxylate (50 mg, 0.10 mmol) and 10% Pd/C (25 mg) in MeOH (5 mL) and EA (5 mL) was stirred at 80° C. for 16 h under hydrogen atmosphere. The mixture was then filtered and concentrated to obtain the desired product as white solid (30 mg, 60%). LCMS (ESI) m/z: 537 [M+H]⁺.

### Step 4: Synthesis of 4-(2-(2,3-dihydrobenzo[b][1,4]dioxin-6-yl)-9-methyl-8-(piperidin-4-yl)-9H-purin-6-yl)morpholine

To a solution of tert-butyl 4-(2-(2,3-dihydrobenzo[b][1,4]dioxin-6-yl)-9-methyl-6-morpholino-9H-purin-8-yl)piperidine-1-carboxylate (30 mg, 0.10 mmol) in DCM (5 mL) was added TFA (2 mL), the mixture was stirred at room temperature for 1 h. It was concentrated and the crude product was purified by prep-HPL C to obtain the desired product as white solid (3.6 mg, 9%).

¹H NMR (400 MHz, DMSO-d₆) δ 7.91-7.86 (m, 2H), 6.92 (d, J=8.4 Hz, 1H), 4.30-4.25 (m, 8H), 3.77-3.75 (m, 7H), 3.33-3.30 (m, 3H), 2.97-3.00 (m, 2H), 2.04-1.93 (m, 4H); LCMS (ESI) m/z: 437.3 [M+H]⁺.

### Synthesis of 4-(9-methyl-8-(pyridin-4-yl)-2-(1,2,3,4-tetrahydroquinolin-7-yl)-9H-purin-6-yl)morpholine (Compound 57)

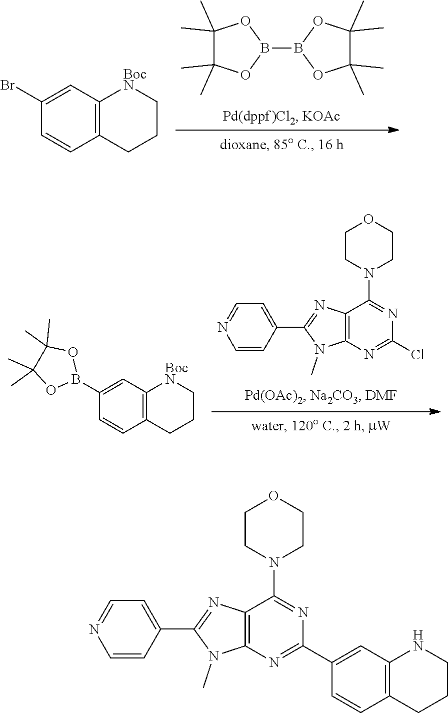

### Step 1: Preparation of tert-butyl 7-(4,4,5,5-tetramethyl-1,3,2-dioxaborolan-2-yl)-3,4-dihydroquinoline-1(2H)-carboxylate

To a solution of tert-butyl 7-bromo-3,4-dihydroquinoline-1(2H)-carboxylate (622 mg, 2 mmol) in dioxane (10 mL) were added 4,4,4′,4′,5,5,5′,5′-octamethyl-2,2′-bi(1,3,2-dioxaborolane) (765 mg, 3 mmol), [1,1′-bis(diphenylphosphino)ferrocene]dichloro palladium (II) (146 mg, 0.2 mmol) and potassium acetate (588 mg, 6 mmol) at 25° C. and the reaction mixture was stirred at 85° C. for 16 h under nitrogen protection. The mixture was then extracted with ethyl acetate (20 mL*2) and washed with water (10 mL*2). The organic layer was dried over sodium sulfate, and concentrated. The crude product was purified by flash chromatography on silica gel (petroleum ether/ester acetic=10:1-3:1) to give crude product tert-butyl 7-(4,4,5,5-tetramethyl-1,3,2-dioxaborolan-2-yl)-3,4-dihydroquinoline-1(2H)-carboxylate as white solid. (610 mg, 84.9%). LCMS (ESI) m/z: 304.2 [M−55]⁺.

### Step 2: Preparation of 4-(9-methyl-8-(pyridin-4-yl)-2-(1,2,3,4-tetrahydroquinolin-7-yl)-9H-purin-6-yl)morpholine

To a solution of 4-(2-chloro-9-methyl-8-(pyridin-4-yl)-9H-purin-6-yl)morpholine (300 mg, 0.83 mmol) in N,N-dimethylformamide (5 mL) was added tert-butyl 7-(4,4,5,5-tetramethyl-1,3,2-dioxaborolan-2-yl)-3,4-dihydroquinoline-1(2H)-carboxylate (132 mg, 0.4 mmol), palladium (II) acetate (20 mg, 0.08 mmol) and sodium carbonate (124 mg, 1.2 mmol) at 25° C. The sealed vial was stirred at 120° C. under microwave for 2 h and the resultant mixture was extracted with ethyl acetate (20 mL*2) and washed with water (10 mL*2). The organic layer was dried over sodium sulfate, and concentrated. The residue (50 mg, 0.1 mmol) was mixed with dichloromethane (5 mL) and trifluoroacetic acid (2 mL), the mixture was stirred at room temperature for 1 h and concentrated. The residue was purified with prep-HPLC (BOSTON pHlex ODS 10 μm 21.2×250 mm 120 A. The mobile phase was dimethyl sulfoxide/0.1% Ammonium bicarbonate) to obtain 4-(9-methyl-8-(pyridin-4-yl)-2-(1,2,3,4-tetrahydroquinolin-7-yl)-9H-purin-6-yl)morpholine as white solid (17.3 mg, 13.3%).

¹H NMR (400 MHz, DMSO-d₆) δ 8.78 (d, J=5.6 Hz, 2H), 7.91 (d, J=5.6 Hz, 2H), 7.62-7.49 (m, 2H), 6.92 (d, J=7.8 Hz, 1H), 5.81 (s, 1H), 4.34 (s, 4H), 3.96 (s, 3H), 3.79 (s, 4H), 3.21 (s, 2H), 2.71 (t, J=5.9 Hz, 2H), 1.82 (s, 2H); LCMS (ESI) m/z: 428.0 [M+H]⁺.

### Synthesis of (4-(9-ethyl-6-morpholino-8-(pyridin-4-yl)-9H-purin-2-yl)-3-(1-methyl-1H-pyrazol-3-yl)phenyl)methanol (Compound 58)

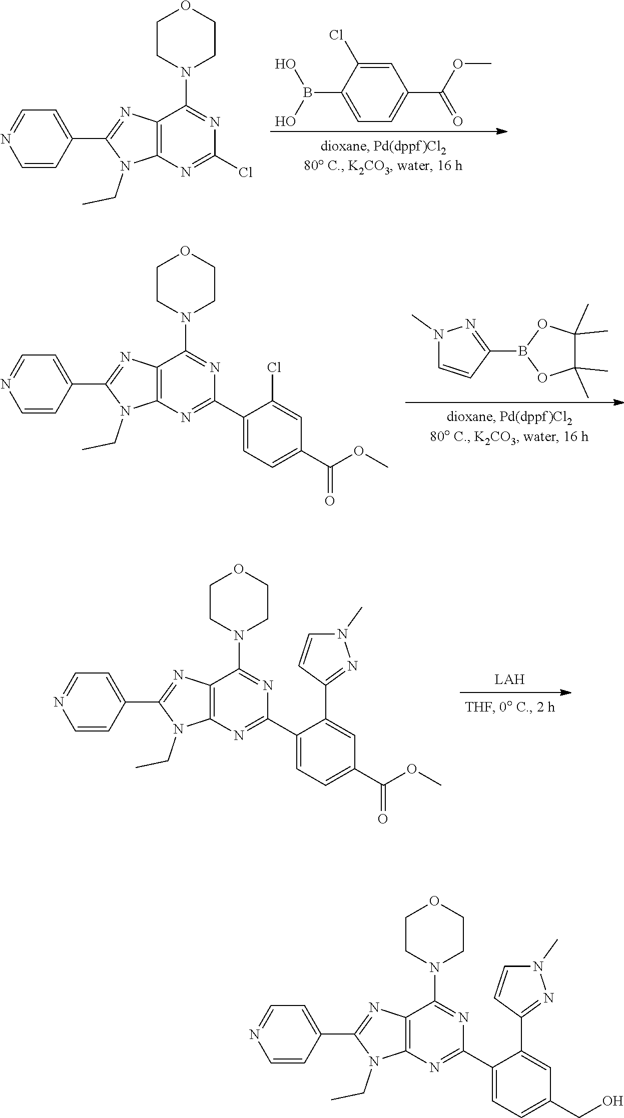

### Step 1: Synthesis of methyl 3-chloro-4-(9-ethyl-6-morpholino-8-(pyridin-4-yl)-9H-purin-2-yl)benzoate

A solution of 4-(2-chloro-9-ethyl-8-(pyridin-4-yl)-9H-purin-6-yl)morpholine (344 mg, 1 mmol), (2-chloro-4-(methoxycarbonyl) phenyl)boronic acid (214 mg, 1 mmol), [1,1′-bis(diphenylphosphino)ferrocene]dichloropalladium(II) (73 mg, 0.1 mmol) and potassium carbonate (73 mg, 0.1 mmol) in dioxane (5 mL) and water (1 mL) was stirred at 80° C. for 16 h under nitrogen. Then water was added and the mixture was extracted with ethyl acetate (50 mL×3). The organic layer was dried and concentrated. The residue was purified by Pre-TLC (petroleum ether:ethyl acetate from 20:1 to 3:1) to give methyl 3-chloro-4-(9-ethyl-6-morpholino-8-(pyridin-4-yl)-9H-purin-2-yl)benzoate (280 mg, 58%) as a white solid. LCMS (ESI) m/z: 479.1 [M+H]⁺.

### Step 2: Synthesis of methyl 4-(9-ethyl-6-morpholino-8-(pyridin-4-yl)-9H-purin-2-yl)-3-(1-methyl-1H-pyrazol-3-yl)benzoate

A solution of methyl 3-chloro-4-(9-ethyl-6-morpholino-8-(pyridin-4-yl)-9H-purin-2-yl)benzoate (478 mg, 1 mmol), 1-methyl-3-(4,4,5,5-tetramethyl-1,3,2-dioxaborolan-2-yl)-1H-pyrazole (312 mg, 1.5 mmol), [1,1′-bis(diphenylphosphino)ferrocene]dichloro palladium(II) (73 mg, 0.1 mmol) and potassium carbonate (27 mg, 2 mmol) in dioxane (5 mL) and water (1 mL) was stirred at 80° C. for 16 h under nitrogen. Then water was added and the mixture was extracted with ethyl acetate (50 mL×3). The organic layer was dried and concentrated. The crude residue was purified by prep-TLC (petroleum ether:ethyl acetate from 50:1 to 10:1) to give methyl 4-(9-ethyl-6-morpholino-8-(pyridin-4-yl)-9H-purin-2-yl)-3-(1-methyl-1H-pyrazol-3-yl)benzoate (450 mg, 85%) as a white solid. LCMS (ESI) m/z: 525.4 [M+H]⁺.

### Step 3: Synthesis of (4-(9-ethyl-6-morpholino-8-(pyridin-4-yl)-9H-purin-2-yl)-3-(1-methyl-1H-pyrazol-3-yl)phenyl)methanol

To a solution of methyl 4-(9-ethyl-6-morpholino-8-(pyridin-4-yl)-9H-purin-2-yl)-3-(1-methyl-1H-pyrazol-3-yl)benzoate (150 mg, 0.28 mmol) in tetrahydrofuran (2 mL) was added lithium aluminum hydride (1 moL/L in tetrahydrofuran, 0.56 mL, 0.56 mmol) slowly at 0° C. After the addition, the mixture was warmed to room temperature and stirred for 2 h. Then water was added and the mixture was extracted with ethyl acetate (50 mL×3). The organic layer was dried and concentrated. The residue was purified by prep-TLC (petroleum ether:ethyl acetate from 50:1 to 10:1) to give (4-(9-ethyl-6-morpholino-8-(pyridin-4-yl)-9H-purin-2-yl)-3-(1-methyl-1H-pyrazol-3-yl)phenyl)methanol (103.2 mg, 74%) as a white solid. 1H NMR (400 MHz, DMSO-d₆) δ 8.79 (dd, J=4.5, 1.6 Hz, 2H), 7.82 (dd, J=4.5, 1.6 Hz, 2H), 7.71 (d, J=7.9 Hz, 1H), 7.58 (d, J=1.2 Hz, 1H), 7.51 (d, J=2.2 Hz, 1H), 7.36 (dd, J=7.9, 1.6 Hz, 1H), 5.66 (d, J=2.2 Hz, 1H), 5.29 (s, 1H), 4.59 (s, 2H), 4.31 (q, J=7.1 Hz, 2H), 4.27-3.80 (m, 4H), 3.79 (s, 3H), 3.64 (s, 4H), 1.25 (t, J=7.2 Hz, 3H); LCMS (ESI) m/z: 497.3 [M+H]⁺.

### Synthesis of 2-(3-(1H-pyrazol-1-yl)phenyl)-9-ethyl-6,8-di(pyridin-4-yl)-9H-purine (Compound 59)

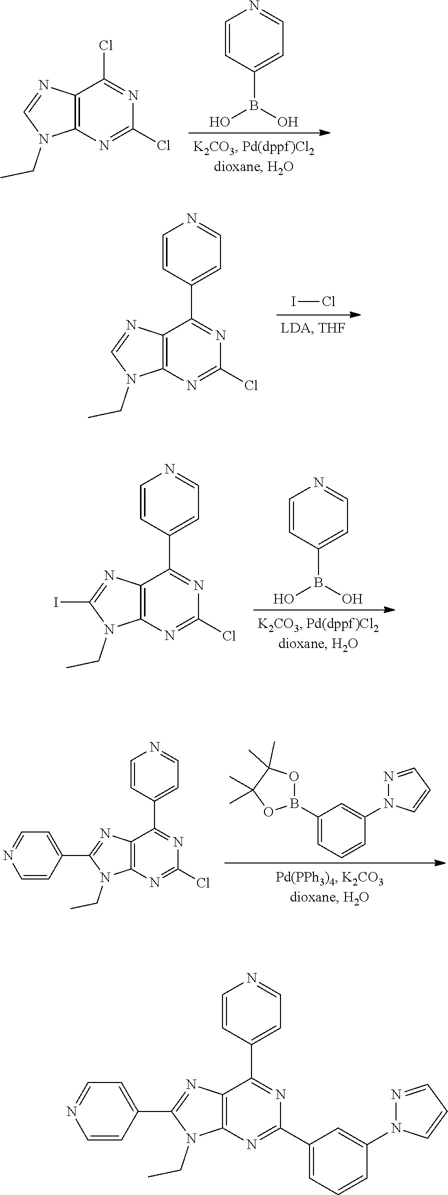

### Step 1: Preparation of 2-chloro-9-ethyl-6-(4-pyridyl)purine

To a solution of 2,6-dichloro-9-ethyl-purine (9.3 g, 42.85 mmol) and 4-pyridylboronic acid (5.27 g, 42.85 mmol) in dioxane (75 mL) and H₂O (25 mL) were added K₂CO₃ (17.76 g, 128.54 mmol) and Pd(dppf)Cl₂ (1.57 g, 2.14 mmol, 0.05 eq). The reaction mixture was stirred at 100° C. for 5 h under nitrogen. The reaction mixture was then cooled to room temperature and quenched by water (75 mL), extracted with ethyl acetate (100 mL*3). The combined organic layers were washed with brine (75 mL), dried over Na₂SO₄, filtered and concentrated under reduced pressure. The crude product thus obtained was purified by flash column (ISCO 80 g silica, 0-10% methanol in dichloromethane, gradient over 20 min) to obtain 2-chloro-9-ethyl-6-(4-pyridyl)purine (4.89 g, 40%) as a purple solid. LCMS (ESI) m/z: 260.2 [M+H]⁺.

### Step 2: Preparation of 2-chloro-9-ethyl-8-iodo-6-(4-pyridyl)purine

To a solution of 2-chloro-9-ethyl-6-(4-pyridyl)purine (4.3 g, 16.56 mmol) in THE (160 mL) was added drop wise LDA (2 M, 16.56 mL) at −70° C.˜−60° C. under nitrogen. The resultant mixture was stirred at −60° C. for 1 h. Then Iodine monochloride (13.44 g, 82.79 mmol) dissolved in THE (83 mL) was added drop wise to the above solution. The resultant reaction mixture was stirred at 20° C. for 2 h, then quenched by 100 mL saturated aqueous sodium thiosulfate and the mixture was extracted with ethyl acetate (150 mL*3). The organic layers were washed with saturated NaHCO₃ aqueous solution (150 mL), water and brine, then dried over Na₂SO₄, filtered and the filtrate was concentrated under reduced pressure. The crude product was purified by flash column chromatography (ISCO 40 g silica, 50-100% ethyl acetate in petroleum ether, gradient over 20 min) to afford 2-chloro-9-ethyl-8-iodo-6-(4-pyridyl)purine (2.45 g, 38%) as brown solid. LCMS (ESI) m/z: 385.9 [M+H]⁺.

### Step 3: Preparation of 2-chloro-9-ethyl-6,8-bis(4-pyridyl)purine

To a solution of 2-chloro-9-ethyl-8-iodo-6-(4-pyridyl)purine (2.3 g, 5.96 mmol) in dioxane (18 mL) and H₂O (6 mL) were added 4-pyridylboronic acid (769 mg, 6.26 mmol), K₂CO₃ (2.47 g, 17.89 mmol) and Pd(dppf)Cl₂ (218 mg, 298 umol). The reaction mixture was stirred at 100° C. for 5 h under nitrogen. It was cooled to room temperature and quenched with water (15 mL) and extracted with ethyl acetate (20 mL*2). The combined organics were washed with brine (15 mL), dried over Na₂SO₄, filtered and concentrated under reduced pressure. The crude product was purified by flash column chromatography (ISCO 20 g silica, 0-10% methanol in dichloromethane, gradient over 20 min) to obtain 2-chloro-9-ethyl-6,8-bis(4-pyridyl)purine (1.6 g, 72%) as yellow solid.

¹H NMR (400 MHz, CHLOROFORM-d) δ 8.93 (br d, J=4.6 Hz, 2H), 8.88 (br s, 2H), 8.72 (d, J=5.5 Hz, 2H), 7.79 (d, J=5.7 Hz, 2H), 4.51 (q, J=7.2 Hz, 2H), 1.54 (t, J=7.2 Hz, 3H).

### Step 4: 9-ethyl-2-(3-pyrazol-1-ylphenyl)-6,8-bis(4-pyridyl)purine

To a solution of 2-chloro-9-ethyl-6,8-bis(4-pyridyl)purine (120 mg, 0.36 mmol) in dioxane (10 mL) were added 1-[3-(4,4,5,5-tetramethyl-1,3,2-dioxaborolan-2-yl)phenyl]pyrazole (96 mg, 0.36 mmol), Pd(PPh₃)₄ (41 mg, 0.04 mmol), H₂O (1 mL) and K₂CO₃ (148 mg, 1.07 mmol). The mixture was stirred at 80° C. for 2 h and then concentrated. The crude product was purified by prep-HPLC (Agela Durashell C18 150*40 10 u column; 30-60% acetonitrile in an a 0.05% ammonia solution in water, 8 mingradient) to obtain 9-ethyl-2-(3-pyrazol-1-ylphenyl)-6,8-bis(4-pyridyl)purine (52 mg, 0.12 mmol, 33%) as a light yellow solid.

¹H NMR (400 MHz, CHLOROFORM-d) δ 9.00 (s, 1H), 8.92-8.84 (m, 6H), 8.66 (d, J=7.8 Hz, 1H), 8.57-8.55 (m, 1H), 8.02-7.98 (m, 3H), 7.73 (s, 1H), 7.73-7.69 (m, 1H), 6.63 (s, 1H) 4.58 (q, J=7.2 Hz, 2H), 1.47 (t, J=7.2 Hz, 3H). LCMS (ESI for C₂₆H₂₀N₈ [M+H]*: 445.2.

### Synthesis of 4-(9-methyl-2-(6-(2-methylpiperidin-4-yl)pyridin-2-yl)-8-(pyridin-4-yl)-9H-purin-6-yl)morpholine (Compound 60)

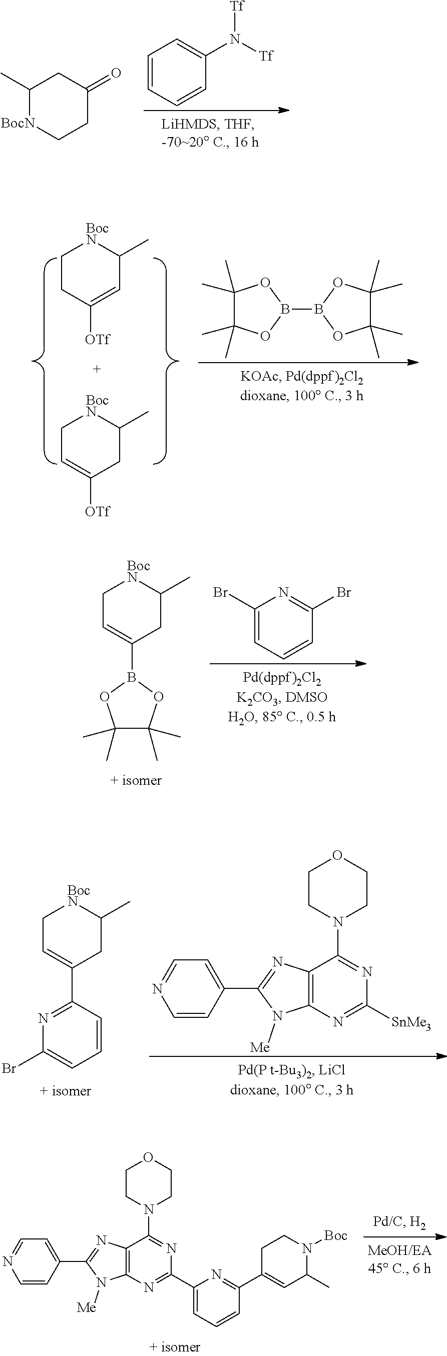

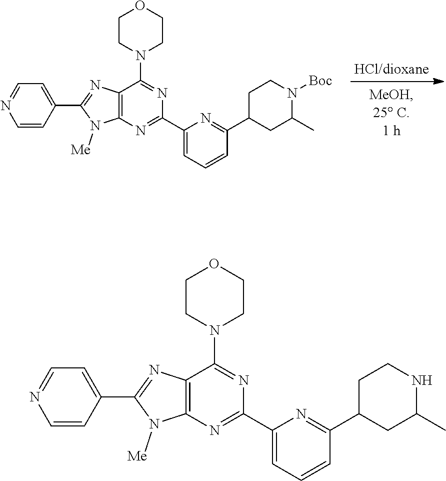 [SMILES: CC1CC(c2cccc(-c3nc(N4CCOCC4)c4nc(-c5ccncc5)n(C)c4n3)n2)CCN1C(=O)OC(C)(C)C ; CC1CC(c2cccc(-c3nc(N4CCOCC4)c4nc(-c5ccncc5)n(C)c4n3)n2)CCN1]

### Step 1: Preparation of tert-butyl 6-methyl-4-(((trifluoromethyl)sulfonyl)oxy)-3,6-dihydropyridine-1(2H)-carboxylate and tert-butyl 2-methyl-4-(((trifluoromethyl)sulfonyl)oxy)-3,6-dihydropyridine-1(2H)-carboxylate

To a solution of tert-butyl 2-methyl-4-oxopiperidine-1-carboxylate (1.5 g, 7 mmol) in tetrahydrofuran (20 mL) was added lithium bis(trimethylsilyl)amide (7.7 mL, 7.7 mmol) at −70° C. slowly. The mixture was stirred at −70° C. for 0.5 h followed by the addition of a solution of 1,1,1-trifluoro-N-phenyl-N-((trifluoromethyl)sulfonyl)methanesulfonamide (2.76 g, 7.7 mmol) in tetrahydrofuran (12 mL) at −70° C., slowly. The mixture was warmed up and stirred at 20° C. for 16 h. Ethyl acetate (50 mL) was added to the reaction mixture and it was washed with aqueous ammonium chloride (20 mL), brine (10 mL), dried over anhydrous sodium sulfate and concentrated. The crude product was purified by flash chromatography (petroleum ether/acetic ester=20:1) to obtain tert-butyl 6-methyl-4-(((trifluoromethyl)sulfonyl)oxy)-3,6-dihydropyridine-1(2H)-carboxylate and tert-butyl 2-methyl-4-(((trifluoromethyl)sulfonyl)oxy)-3,6-dihydropyridine-1(2H)-carboxylate (1.4 g, 58%) as a light yellow oil. LCMS (ESI) m/z: 290.1 [M+H−56]⁺.

### Step 2: Preparation of tert-butyl 2-methyl-4-(4,4,5,5-tetramethyl-1,3,2-dioxaborolan-2-yl)-3,6-dihydropyridine-1(2H)-carboxylate and tert-butyl 6-methyl-4-(4,4,5,5-tetramethyl-1,3,2-dioxaborolan-2-yl)-3,6-dihydropyridine-1(2H)-carboxylate

To a solution of tert-butyl 6-methyl-4-(((trifluoromethyl)sulfonyl)oxy)-3,6-dihydropyridine-1(2H)-carboxylate and tert-butyl 2-methyl-4-(((trifluoromethyl)sulfonyl)oxy)-3,6-dihydropyridine-1(2H)-carboxylate (1.45 g, 4.2 mmol) and 4,4,4′,4′,5,5,5′,5′-octamethyl-2,2′-bi(1,3,2-dioxaborolane) (1.17 g, 4.62 mmol) in dioxane (25 mL) were added potassium acetate (0.82 g, 8.4 mmol) and [1,1′-bis(diphenylphosphino)ferrocene]dichloropalladium(II) (0.31 g, 0.42 mmol) and the resultant mixture was stirred at 100° C. under nitrogen for 3 h. The mixture was then concentrated and purified by (petroleum ether:ethyl acetate=10:1) to give tert-butyl 2-methyl-4-(4,4,5,5-tetramethyl-1,3,2-dioxaborolan-2-yl)-3,6-dihydropyridine-1(2H)-carboxylate and tert-butyl 6-methyl-4-(4,4,5,5-tetramethyl-1,3,2-dioxaborolan-2-yl)-3,6-dihydropyridine-1(2H)-carboxylate (650 mg, 48%) as a white solid. LCMS (ESI) m/z: 268.2 [M+H−56]+.

### Step 3: Preparation of tert-butyl 6-bromo-2′-methyl-3′,6′-dihydro-[2,4′-bipyridine]-1′(2′H)-carboxylate and tert-butyl 6-bromo-6′-methyl-3′,6′-dihydro-[2,4′-bipyridine]-1′(2′H)-carboxylate

To a solution of tert-butyl 2-methyl-4-(4,4,5,5-tetramethyl-1,3,2-dioxaborolan-2-yl)-3,6-dihydropyridine-1(2H)-carboxylate and tert-butyl 6-methyl-4-(4,4,5,5-tetramethyl-1,3,2-dioxaborolan-2-yl)-3,6-dihydropyridine-1(2H)-carboxylate (0.5 g, 1.55 mmol) and 2,6-dibromopyridine (0.5 g, 2.1 mmol) in DMSO/water (17 mL/1.8 mL) were added potassium carbonate (0.64 g, 4.64 mmol) and [1,1′-bis(diphenylphosphino)ferrocene]dichloropalladium(II) (0.117 g, 0.16 mmol) and the reaction mixture was stirred at 85° C. under nitrogen for 0.5 h. The reaction was quenched with water (50 mL) and extracted with ethyl acetate (30 mL*2) and the organics was concentrated. The crude product was purified by SGC (petroleum ether:ethyl acetate=10:1) to give tert-butyl 6-bromo-2′-methyl-3′,6′-dihydro-[2,4′-bipyridine]-1′(2′H)-carboxylate and tert-butyl 6-bromo-6′-methyl-3′,6′-dihydro-[2,4′-bipyridine]-1′(2′H)-carboxylate (410 mg, 75%) as a light yellow oil. LCMS (ESI) m/z: 297.1 [M+H−56]+.

### Step 4: Preparation of tert-butyl 6′-methyl-6-(9-methyl-6-morpholino-8-(pyridin-4-yl)-9H-purin-2-yl)-3′,6′-dihydro-[2,4′-bipyridine]-1′(2′H)-carboxylate and tert-butyl 2′-methyl-6-(9-methyl-6-morpholino-8-(pyridin-4-yl)-9H-purin-2-yl)-3′,6′-dihydro-[2,4′-bipyridine]-1′(2′H)-carboxylate

To a solution of tert-butyl 6-bromo-6′-methyl-3′,6′-dihydro-[2,4′-bipyridine]-1′(2′H)-carboxylate and tert-butyl 6-bromo-2′-methyl-3′,6′-dihydro-[2,4′-bipyridine]-1′(2′H)-carboxylate (0.33 g, 0.72 mmol) in dioxane (8 mL) were added lithium chloride (0.06 g, 1.4 mmol), 4-(9-methyl-8-(pyridin-4-yl)-2-(trimethylstannyl)-9H-purin-6-yl)morpholine (0.3 g, 0.86 mmol) and bis(tri-tert-butylphosphine)palladium(0) (0.08 g, 0.072 mmol) and the reaction was stirred at 100° C. under nitrogen for 4 h. The reaction was quenched with aqueous potassium fluoride (15 mL), filtered and extracted with dichloromethane (20 mL*3). The pooled organic layer was concentrated and the resultant crude product was purified by SGC (petroleum ether:ethyl acetate=2:1) to give mixture of tert-butyl 2′-methyl-6-(9-methyl-6-morpholino-8-(pyridin-4-yl)-9H-purin-2-yl)-3′,6′-dihydro-[2,4′-bipyridine]-1′(2′H)-carboxylate and tert-butyl 6′-methyl-6-(9-methyl-6-morpholino-8-(pyridin-4-yl)-9H-purin-2-yl)-3′,6′-dihydro-[2,4′-bipyridine]-1′(2′H)-carboxylate (100 mg, 24%) as a yellow solid. LCMS (ESI) m/z: 569.3 [M+H]+.

### Step 5: Preparation of tert-butyl 2-methyl-4-(6-(9-methyl-6-morpholino-8-(pyridin-4-yl)-9H-purin-2-yl)pyridin-2-yl)piperidine-1-carboxylate

A mixture of tert-butyl 2-methyl-4-(4,4,5,5-tetramethyl-1,3,2-dioxaborolan-2-yl)-3,6-dihydropyridine-1(2H)-carboxylate and tert-butyl 6-methyl-4-(4,4,5,5-tetramethyl-1,3,2-dioxaborolan-2-yl)-3,6-dihydropyridine-1(2H)-carboxylate (0.085 g, 0.15 mmol) and palladium on activated carbon (10% Pd, 0.07 g) in Methanol/ethyl acetate (4 mL/4 mL) was stirred at 45° C. under hydrogen for 6 h. The reaction was filtered and concentrated to give tert-butyl 2-methyl-4-(6-(9-methyl-6-morpholino-8-(pyridin-4-yl)-9H-purin-2-yl)pyridin-2-yl)piperidine-1-carboxylate (40 mg, 47%) as a light yellow solid. LCMS (ESI) m/z: 571.3 [M+H]+.

### Step 6: Preparation of 4-(9-methyl-2-(6-(2-methylpiperidin-4-yl)pyridin-2-yl)-8-(pyridin-4-yl)-9H-purin-6-yl)morpholine

A mixture of tert-butyl 2-methyl-4-(6-(9-methyl-6-morpholino-8-(pyridin-4-yl)-9H-purin-2-yl)pyridin-2-yl)piperidine-1-carboxylate (40 mg, 0.07 mmol), hydrochloric acid/dioxane (4 mL) and methanol (1 mL) was stirred at 25° C. for 1 hour. The mixture was filtered and purified by Prep-HPLC (SunFire C18, 4.6*50 mm, 3.5 um column Xbridge C18 3.5 μm 4.6×50 mm column. The mobile phase was acetonitrile/10 mM ammonium bicarbonate aqueous solution.) to obtain 4-(9-methyl-2-(6-(2-methylpiperidin-4-yl)pyridin-2-yl)-8-(pyridin-4-yl)-9H-purin-6-yl)morpholine as white solid. (5.2 mg, 16%).

¹H NMR (400 MHz, DMSO) δ 8.80 (d, J=5.6 Hz, 2H), 8.20 (d, J=8.0 Hz, 1H), 7.95 (d, J=5.4 Hz, 2H), 7.86 (t, J=7.7 Hz, 1H), 7.34 (d, J=7.6 Hz, 1H), 4.36 (s, 4H), 4.01 (s, 3H), 3.80 (s, 4H), 3.14 (d, J=10.1 Hz, 1H), 2.93 (s, 1H), 2.79 (d, J=11.7 Hz, 1H), 2.15-2.05 (m, 1H), 1.95-1.85 (m, 2H), 1.75-1.60 (m, 1H), 1.40-1.30 (m, 1H), 1.10-1.05 (m, 3H); LCMS (ESI) m/z: 471.3 [M+H]⁺.

### Synthesis of 8-(2-methoxypyridin-4-yl)-9-methyl-6-(piperidin-1-yl)-2-(1-(2,2,2-trifluoroethyl)-1H-pyrazol-3-yl)-9H-purine (Compound 61)

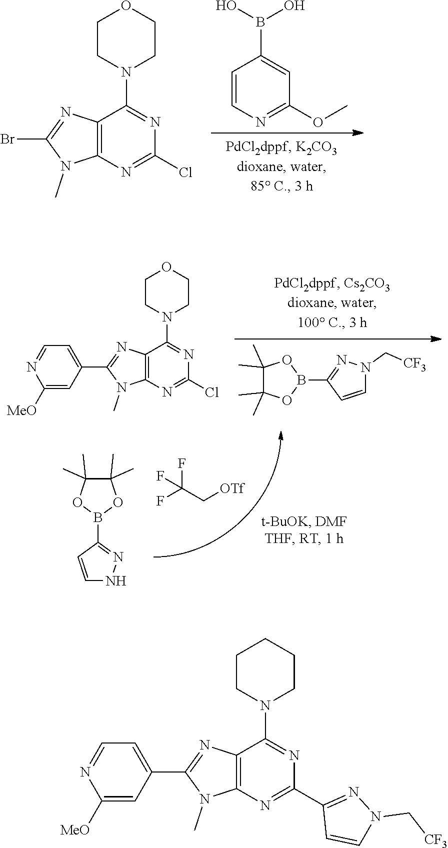

### Step 1a: Synthesis of 3-(4,4,5,5-tetramethyl-1,3,2-dioxaborolan-2-yl)-1-(2,2,2-trifluoroethyl)-1H-pyrazole

To a solution of 3-(4,4,5,5-tetramethyl-1,3,2-dioxaborolan-2-yl)-1H-pyrazole (194 mg, 1 mmol) in N,N-dimethylformamide (5 mL) and tetrahydrofuran (5 mL) were added 2,2,2-trifluoroethyl trifluoromethanesulfonate (696 mg, 1 mmol) and potassium t-butoxide (22 mg, 0.1 mmol) at 25° C. The resultant reaction mixture was stirred at r.t for 1 h, then diluted with water (30 mL) and extracted with ethyl acetate (20 mL*3). The organic layer was dried over sodium sulfate, filtered and concentrated to afford the crude product 3-(4,4,5,5-tetramethyl-1,3,2-dioxaborolan-2-yl)-1-(2,2,2-trifluoroethyl)-1H-pyrazole (250 mg, 90.5%). LCMS (ESI) m/z: 277.0 [M+H]⁺.

### Step 1 b: Synthesis of 4-(2-chloro-8-(2-methoxypyridin-4-yl)-9-methyl-9H-purin-6-yl)morpholine

To a solution of 4-(8-bromo-2-chloro-9-methyl-9H-purin-6-yl)morpholine (330 mg, 1 mmol) in dioxane (9 mL) and water (1 mL) was added (2-methoxypyridin-4-yl)boronic acid (150 mg, 1 mmol), [1,1′-bis(diphenylphosphino)ferrocene]dichloropalladium(II) (73 mg, 0.1 mmol) and potassium carbonate (414 mg, 3 mmol) at 25° C. The reaction mixture was stirred at 85° C. for 3 h under argon atmosphere. The products were then extracted with ethyl acetate (20 mL*2), washed with water (10 mL*2), dried over sodium sulfate, and concentrated. The crude product was purified by flash chromatography on silica gel (petroleum ether/ester acetic 3:1→1:1) to give 4-(2-chloro-8-(2-methoxypyridin-4-yl)-9-methyl-9H-purin-6-yl)morpholine as a white solid. (110 mg, 30.6%). LCMS (ESI) m/z: 361.1 [M+H]⁺.

### Step 2: Synthesis of 8-(2-methoxypyridin-4-yl)-9-methyl-6-(piperidin-1-yl)-2-(1-(2,2,2-trifluoroethyl)-1H-pyrazol-3-yl)-9H-purine

To a solution of 4-(2-chloro-8-(2-methoxypyridin-4-yl)-9-methyl-9H-purin-6-yl)morpholine (83 mg, 0.3 mmol) in dioxane (9 mL) and water (1 mL) was added 3-(4,4,5,5-tetramethyl-1,3,2-dioxaborolan-2-yl)-1-(2,2,2-trifluoroethyl)-1H-pyrazole (110 mg, 0.3 mmol), [1,1′-bis(diphenylphosphino)ferrocene]dichloropalladium(II) (21 mg, 0.03 mmol) and potassium carbonate (124 mg, 0.9 mmol) at 25° C. The resultant mixture was stirred at 100° C. for 3 h under argon atmosphere. The products were extracted with ethyl acetate (20 mL*2), washed with water (10 mL*2), dried and concentrated. The residue was purified by Prep-HPLC (BOSTON pHlex ODS 10 μm 21.2ᵢÁ250 mm120 A. The mobile phase was acetonitrile/0.1% Formic acid) to give 8-(2-methoxypyridin-4-yl)-9-methyl-6-(piperidin-1-yl)-2-(1-(2,2,2-trifluoroethyl)-1H-pyrazol-3-yl)-9H-purine as a yellow solid. (15.0 mg, 10.5%). ¹H NMR (400 MHz, DMSO-d₆) δ 8.36 (d, J=5.3 Hz, 1H), 7.91 (d, J=2.3 Hz, 1H), 7.50 (dd, J=5.3, 1H), 7.31 (s, 1H), 7.02 (d, J=2.3 Hz, 1H), 5.27 (q, J=9.2 Hz, 2H), 4.32 (m, 4H), 3.93 (d, J=4.2 Hz, 6H), 3.82-3.73 (m, 4H); LCMS (ESI) m/z: 475.1 [M+H]⁺.

### Synthesis of 3-methyl-4-(9-methyl-2-(1-phenyl-1H-pyrazol-3-yl)-8-(pyridin-4-yl)-9H-purin-6-yl)morpholine (Compound 62)

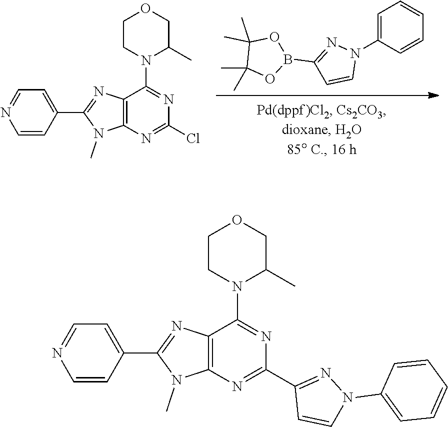

A mixture of 4-(2-chloro-9-methyl-8-(pyridin-4-yl)-9H-purin-6-yl)-3-methylmorpholine (200 mg, 0.58 mmol), 1-phenyl-3-(4,4,5,5-tetramethyl-1,3,2-dioxaborolan-2-yl)-1H-pyrazole (187 mg, 0.69 mmol), [1,1′-bis(diphenylphosphino)ferrocene]dichloropalladium (II) (42 mg, 0.058 mmol) and potassium carbonate (473 mg, 1.45 mmol) in dioxane (5 mL) and water (0.5 mL) under nitrogen protection was stirred at 85° C. for 3 h. The mixture was filtered and the filtrate was concentrated. The resultant crude product was purified by prep-HPLC to give the target compound (159 mg, 60%) as a white solid. 1H NMR (400 MHz, CDCl3) δ 8.80 (d, J=6.1 Hz, 2H), 8.01 (d, J=2.5 Hz, 1H), 7.86 (d, J=6.9 Hz, 2H), 7.79 (d, J=4 Hz, 2H), 7.49 (dd, J=15.3, 7.8 Hz, 2H), 7.32 (t, J=7.4 Hz, 1H), 7.23 (d, J=2.4 Hz, 1H), 5.98-5.43 (m, 1H), 5.46-4.73 (m, 1H), 4.10 (d, J=8.1 Hz, 1H), 4.08 (s, 3H), 3.88 (s, 2H), 3.73 (t, J=10.5 Hz, 1H), 3.61 (s, 1H), 1.49 (d, J=6.8 Hz, 3H); LCMS (ESI) m/z: 453.1 [M+H]⁺.

### Synthesis of 4-(2-(1-(cyclobutylmethyl)-1H-pyrazol-5-yl)-9-ethyl-8-(pyridin-4-yl)-9H-purin-6-yl)morpholine (Compound 63)

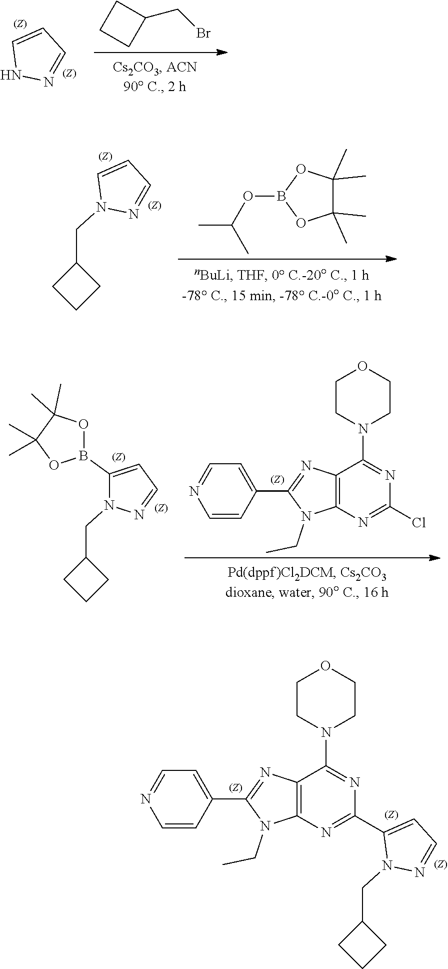

### Step 1: Synthesis of 1-(cyclobutylmethyl)-1H-pyrazole

A mixture of 1H-pyrazole (1.36 g, 20 mmol), (bromomethyl)cyclobutane (3.576 g, 24 mmol) and cesium carbonate (13.04 g, 40 mmol) in acetonitrile (40 mL) was stirred at 90° C. for 2 h. The reaction mixture was concentrated and residue was diluted with water (50 mL) and extracted with ethyl acetate (100 mL*2). The combined organic layer was dried over sodium sulfate, filtered and concentrated. The residue was purified by silica gel chromatography eluting with a linear gradient of 0% to 30% ethyl acetate in petroleum ether to get (2.6 g, 91%) as a yellow oil. LCMS: [M+H]⁺=137.3.

### Step 2: Synthesis of 1-(cyclobutylmethyl)-5-(4,4,5,5-tetramethyl-1,3,2-dioxaborolan-2-yl)-1H-pyrazole

To a solution of 1-(cyclobutylmethyl)-1H-pyrazole (1.36 g, 10 mmol) in tetrahydrofuran (30 mL) at 0° C. was added n-butyllithium (2.5 M in tetrahydrofuran, 4.4 mL, 11 mmol). The reaction mixture was stirred for 1 h at 20° C. and then cooled to −78° C. To the resultant mixture was added 2-isopropoxy-4,4,5,5-tetramethyl-1,3,2-dioxaborolane (2.23 g, 12 mmol) and stirred for 15 min at −78° C. and the reaction mixture was allowed to warm to 0° C. over 1 h. The reaction mixture was diluted with sat. aq. Ammonium chloride solution (20 mL) and extracted with dichloromethane (80 mL×2). The organic fractions were washed with water (50 mL×2), dried over sodium sulfate and concentrated in vacuo to afford the product 1-(cyclobutylmethyl)-5-(4,4,5,5-tetramethyl-1,3,2-dioxaborolan-2-yl)-1H-pyrazole (3.2 g, crude) as a yellow oil. It was directly used in the next step without further purification. LCMS: [M+H]*=263.3.

### Step 3: Synthesis of 4-(2-(1-(cyclobutylmethyl)-1H-pyrazol-5-yl)-9-ethyl-8-(pyridin-4-yl)-9H-purin-6-yl)morpholine

A solution of 4-(2-chloro-9-ethyl-8-(pyridin-4-yl)-9H-purin-6-yl)morpholine (69 mg, 0.2 mmol), 1-(cyclobutylmethyl)-5-(4,4,5,5-tetramethyl-1,3,2-dioxaborolan-2-yl)-1H-pyrazole (157 mg, 0.6 mmol), 1,1′-bis(diphenylphosphino)ferrocene-palladium(II) dichloride dichloromethane complex (16 mg, 0.02 mmol) and cesium carbonate (261 mg, 0.8 mmol) in water (1 mL) and dioxane (10 mL) was stirred at 90° C. for 16 h under argon. The mixture was filtered, the filtrate was concentrated and purified by pre-HPLC (BOSTON pHlex ODS 10 μm 21.2×250 mm 120 A. The mobile phase was acetonitrile/0.1% Ammonium bicarbonate) to afford 4-(2-(1-(cyclobutylmethyl)-1H-pyrazol-5-yl)-9-ethyl-8-(pyridin-4-yl)-9H-purin-6-yl)morpholine (36.1 mg, 32.5%) as a white solid.

1H NMR (400 MHz, DMSO-d₆) δ 8.80 (d, J=5.9 Hz, 2H), 7.83 (d, J=6.0 Hz, 2H), 7.47 (d, J=1.8 Hz, 1H), 6.97 (d, J=1.8 Hz, 1H), 4.86 (d, J=7.2 Hz, 2H), 4.40 (q, J=7.2 Hz, 2H), 4.29 (bs, 4H), 3.84-3.72 (m, 4H), 2.85 (dd, J=15.0, 7.4 Hz, 1H), 1.95-1.78 (, 6H), 1.39 (t, J=7.1 Hz, 3H); LCMS: (ESI) m/z: 445.2 [M+H]⁺.

The following compounds were synthesized according to the protocol described above:

| Name | Structure | NMR, MS | # |
|---|---|---|---|
| 4-(2-(1-ethyl-1H- pyrazol-3-yl)-8-(2- methoxypyridin-4- yl)-9-methyl-9H- purin-6- yl)morpholine | 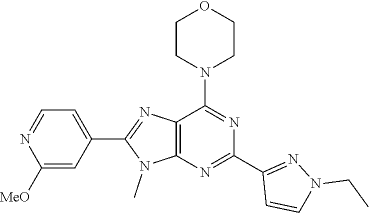 [SMILES: CCn1ccc(-c2nc(N3CCOCC3)c3nc(-c4ccnc(OC)c4)n(C)c3n2)n1] | ¹H NMR (400 MHz, CDCl₃) δ 8.31 (d, J = 5.3 Hz, 1H), 7.46 (s, 1H), 7.33 (d, J = 4.9 Hz, 1H), 7.18 (s, 1H), 7.00 (d, J = 1.8 Hz, 1H), 4.42 (s, 4H), 4.29-4.34 (m, 2H), 4.01 (s, 6H), 3.90-3.86 (m, 4H), 1.56 (t, J = 7.3 Hz, 3H); LCMS (ESI) m/z: 421.2 [M + H]⁺. | 64 |
| 4-(9-methyl-8- (pyridin-4-yl)-2-(1- (2,2,2- trifluoroethyl)-1H- pyrazol-3-yl)-9H- purin-6- yl)morpholine | 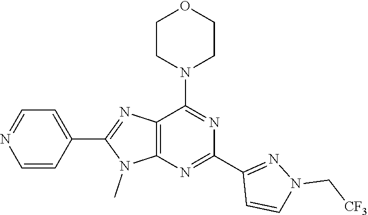 [SMILES: Cn1c(-c2ccncc2)nc2c(N3CCOCC3)nc(-c3ccn(CC(F)(F)F)n3)nc21] | ¹H NMR (500 MHz, DMSO-d₆) δ 8.78 (t, J = 4.8 Hz, 2H), 7.92 (d, J = 5.8 Hz, 3H), 7.02 (d, J = 2.3 Hz, 1H), 5.27 (q, J = 9.2 Hz, 2H), 4.32 (s, 4H), 3.95 (s, 3H), 3.83-3.73 (m, 4H); LCMS (ESI) m/z: 445.0 [M + H]⁺. | 65 |
| 4-(2-(1-ethyl-1H- pyrazol-3-yl)-9- methyl-8-(pyridin-4- yl)-9H-purin-6- yl)morpholine | 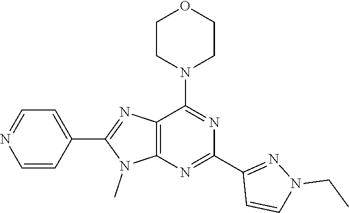 [SMILES: CCn1ccc(-c2nc(N3CCOCC3)c3nc(-c4ccncc4)n(C)c3n2)n1] | ¹H NMR (400 MHz, CDCl₃) δ 8.79 (d, J = 5.9 Hz, 2H), 7.76 (d, J = 4.5 Hz, 2H), 7.47 (d, J = 2.4 Hz, 1H), 7.01 (d, J = 2.4 Hz, 1H), 4.43-4.30 (m, 6H), 4.04 (s, 3H), 3.92-3.87 (m, 4H), 1.57 (t, J = 5.9 Hz, 3H); LCMS (ESI) m/z: 391.2 [M + H]⁺. | 66 |
| 4-(9-ethyl-2-(2- methyl-1H- imidazol-5-yl)-8- (pyridin-4-yl)-9H- purin-6- yl)morpholine | 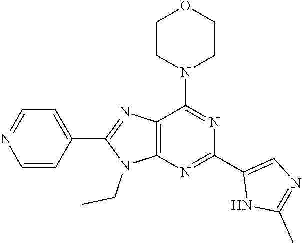 [SMILES: CCn1c(-c2ccncc2)nc2c(N3CCOCC3)nc(-c3cnc(C)[nH]3)nc21] | 1H NMR (400 MHz, CDCl₃) δ 8.79 (dd, J = 4.5, 1.6 Hz, 2H), 7.71-7.67 (m, 3H), 4.59-4.20 (m, 6H), 3.90-3.85 (m, 4H), 2.54 (s, 3H), 1.47 (t, J = 7.2 Hz, 3H); LCMS: [M + H]⁺ = 390.9 | 67 |
| 4-(9-ethyl-2-(1- phenyl-1H-pyrazol- 3-yl)-8-(pyridin-4- yl)-9H-purin-6-yl)-3- methylmorpholine | 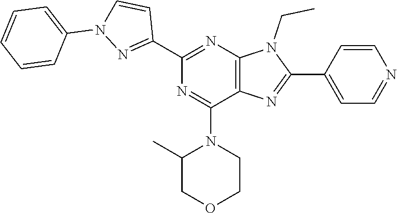 [SMILES: CCn1c(-c2ccncc2)nc2c(N3CCOCC3C)nc(-c3ccn(-c4ccccc4)n3)nc21] | ¹HNMR(400 MHz, DMSO) δ 8.81 (dd, J = 4.5, 1.5 Hz, 2H), 8.59 (d, J = 2.5 Hz, 1H), 7.95 (d, J = 7.6 Hz, 2H), 7.88 (dd, J = 4.5, 1.6 Hz, 2H), 7.56 (t, J = 8.0 Hz, 2H), 7.36 (t, J = 7.4 Hz, 1H), 7.18 (d, J = 2.5 Hz, 1H), 5.78-4.82 (bs, 2H), 4.47 (q, J = 7.2 Hz, 2H), 4.03 (d, J = 8.6 Hz, 1H), 3.83 (d, J = 11.3 Hz, 1H), 3.75 (s, 1H), 3.57 (d, J = 11.4 Hz, 2H), 1.39-1.34 (m, 6H); LCMS (ESI) m/z: 467.3 [M + H]⁺ | 68 |
| 4-(9-ethyl-2-(1- phenyl-1H-pyrazol- 4-yl)-8-(pyridin-4- yl)-9H-purin-6- yl)morpholine | 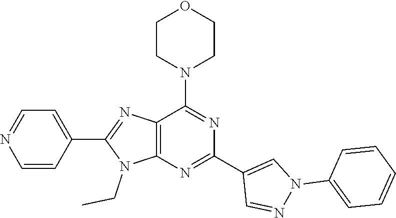 [SMILES: CCn1c(-c2ccncc2)nc2c(N3CCOCC3)nc(-c3cnn(-c4ccccc4)c3)nc21] | ¹H NMR (400 MHz, DMSO-d₆) δ 9.06 (s, 1H), 8.79 (d, J = 5.7 Hz, 2H), 8.33 (s, 1H), 7.99 (d, J = 7.7 Hz, 2H), 7.85 (d, J = 6.0 Hz, 2H), 7.54 (t, J = 8.0 Hz, 2H), 7.36 (t, J = 7.4 Hz, 1H), 4.47 (q, J = 7.2 Hz, 2H), 4.36 (bs, 4H), 3.87-3.72 (m, 4H), 1.36 (t, J = 7.2 Hz, 3H); LCMS (ESI) m/z: 453.0 [M + H]+. | 69 |
| 4-(9-ethyl-2-(1- phenyl-1H-pyrazol- 4-yl)-8-(pyridin-4- yl)-9H-purin-6-yl)-3- methylmorpholine | 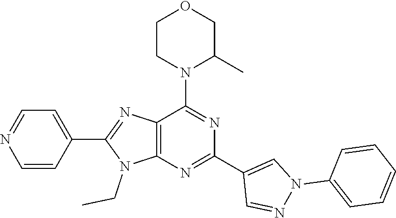 [SMILES: CCn1c(-c2ccncc2)nc2c(N3CCOCC3C)nc(-c3cnn(-c4ccccc4)c3)nc21] | ¹H NMR (400 MHz, DMSO-d₆) δ 9.04 (s, 1H), 8.80 (d, J = 6.0 Hz, 2H), 8.32 (s, 1H), 7.99 (d, J = 7.6 Hz, 2H), 7.86 (d, J = 6.1 Hz, 2H), 7.58- 7.49 (m, 2H), 7.36 (t, J = 7.4 Hz, 1H), 4.47 (q, J = 7.2 Hz, 2H), 4.03 (d, J = 8.3 Hz, 1H), 3.83 (d, J = 12 Hz, 1H), 3.74 (d, J = 12 Hz, 1H), 3.57 (d, J = 11.3 Hz, 1H), 1.45-1.35 (m, 6H); LCMS (ESI) m/z: 467.0 [M + H]+. | 70 |
| 4-(9-ethyl-2-(3-(1- methyl-1H-pyrazol- 3-yl)phenyl)-8- (pyridin-4-yl)-9H- purin-6- yl)morpholine | 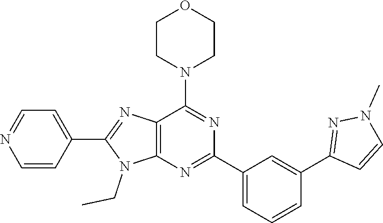 [SMILES: CCn1c(-c2ccncc2)nc2c(N3CCOCC3)nc(-c3cccc(-c4ccn(C)n4)c3)nc21] | ¹H NMR (400 MHz, DMSO-d₆) δ 8.81 (dd, J = 4.5, 1.6 Hz, 3H), 8.35 (d, J = 7.9 Hz, 1H), 7.90-7.85 (m, 3H), 7.78 (d, J = 2.2 Hz, 1H), 7.52 (t, J = 7.7 Hz, 1H), 6.76 (d, J = 2.2 Hz, 1H), 4.53-4.27 (m, 6H), 3.93 (s, 3H), 3.85-3.76 (m, 4H), 1.39 (t, J = 7.2 Hz, 3H); LCMS (ESI) m/z: 467.0 [M + H]+. | 71 |
| 4-(2-(1-benzyl-1H- pyrazol-3-yl)-9- ethyl-8-(pyridin-4- yl)-9H-purin-6- yl)morpholine | 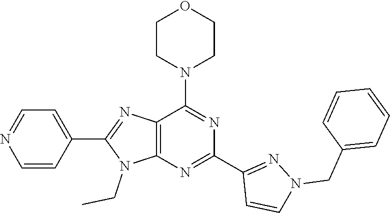 [SMILES: CCn1c(-c2ccncc2)nc2c(N3CCOCC3)nc(-c3ccn(Cc4ccccc4)n3)nc21] | ¹H NMR (400 MHz, DMSO-d₆) δ 8.79 (d, J = 5.9 Hz, 2H), 7.89 (d, J = 2.2 Hz, 1H), 7.84 (d, J = 6.0 Hz, 2H), 7.40-7.24 (m, 5H), 6.95 (d, J = 2.2 Hz, 1H), 5.46 (s, 2H), 4.44 (q, J = 7.2 Hz, 2H), 4.30 (s, 4H), 3.79-3.74 (m, 4H), 1.31 (t, J = 7.2 Hz, 3H); LCMS (ESI) m/z: 467.2 [M + H]+. | 72 |
| 4-(9-cyclopropyl-2- (1-cyclopropyl-1H- pyrazol-5-yl)-8- (pyridin-4-yl)-9H- purin-6- yl)morpholine | 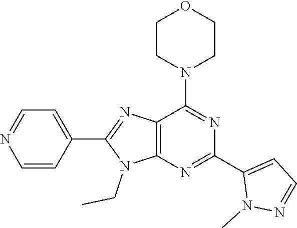 | ¹H NMR (400 MHz, DMSO-d₆) δ 8.78 (d, J = 6.1 Hz, 2H), 8.06-7.98 (m, 2H), 7.43 (d, J = 1.8 Hz, 1H), 6.92 (d, J = 1.8 Hz, 1H), 4.85 (dd, J = 7.3, 3.7 Hz, 1H), 4.30 (bs, 4H), 3.77 (d, J = 4.3 Hz, 5H), 1.13 (d, J = 4.6 Hz, 4H), 1.06-0.99 (m, 2H), 0.84 (s, 2H); LCMS (ESI) m/z: 428.9 [M + H]+. | 73 |
| 4-(2-(1- (cyclopropylmethyl)- 1H-pyrazol-5-yl)-9- ethyl-8-(pyridin-4- yl)-9H-purin-6- yl)morpholine | 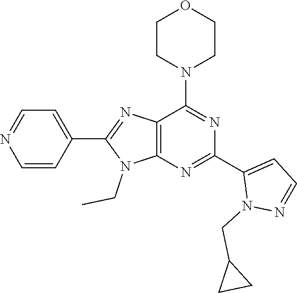 [SMILES: CCn1c(-c2ccncc2)nc2c(N3CCOCC3)nc(-c3ccnn3CC3CC3)nc21] | H NMR (500 MHz, CDCl3) δ 8.81 (d, J = 5.6 Hz, 2H), 7.69 (d, J = 5.6 Hz, 2H), 7.54 (d, J = 1.3 Hz, 1H), 7.04 (d, J = 1.3 Hz, 1H), 4.75 (d, J = 7.0 Hz, 2H), 4.60-4.25 (m, 6H), 3.92-3.84 (m, 4H), 1.55- 1.45 (m, 4H), 0.54-0.47 (m, 2H), 0.44 (t, J = 4.4 Hz, 2H); LCMS: (ESI) m/z: 430.8 [M + H]⁺. | 74 |
| (R)-4-(2-(1- cyclopropyl-1H- pyrazol-5-yl)-9- ethyl-8-(pyridin-4- yl)-9H-purin-6-yl)-3- methylmorpholine | 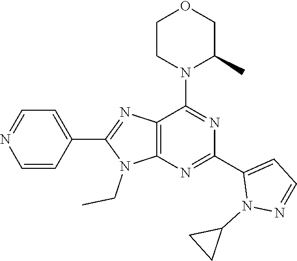 [SMILES: CCn1c(-c2ccncc2)nc2c(N3CCOC[C@H]3C)nc(-c3ccnn3C3CC3)nc21] | ¹H NMR (400 MHz, DMSO-d₆) δ 8.81 (dd, J = 4.5, 1.5 Hz, 2H), 7.85 (dd, J = 4.5, 1.6 Hz, 2H), 7.43 (d, J = 1.8 Hz, 1H), 6.91 (d, J = 1.8 Hz, 1H), 5.36 (s, 2H), 4.68 (dd, J = 7.4, 3.5 Hz, 1H), 4.41 (q, J = 7.1 Hz, 2H), 4.01 (d, J = 8.3 Hz, 1H), 3.90 (d, J = 12 Hz, 1H), 3.72 (d, J = 11.4 Hz, 1H), 3.56 (d, J = 10.5 Hz, 2H), 1.37 (t, J = 7.6 Hz, 6H), 1.14 (s, 2H), 1.02 (dd, J = 7.4, 1.6 Hz, 2H); LCMS (ESI) m/z: 430.7 [M+]⁺. | 75 |
| 4-(9-ethyl-2-(1H- pyrazol-3-yl)-8- (pyridin-4-yl)-9H- purin-6- yl)morpholine | 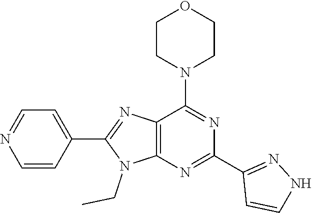 [SMILES: CCn1c(-c2ccncc2)nc2c(N3CCOCC3)nc(-c3cc[nH]n3)nc21] | ¹H NMR (400 MHz, DMSO) δ 13.48 (s, 1H), 8.79 (dd, J = 4.5, 1.6 Hz, 2H), 7.84 (dd, J = 4.5, 1.6 Hz, 2H), 7.61 (s, 1H), 6.89 (d, J = 1.8 Hz, 1H), 4.45 (q, J = 7.2 Hz, 2H), 4.36 (bs, 4H), 3.86- 3.70 (m, 4H), 1.34 (t, J = 7.2 Hz, 3H). LCMS (ESI) m/z: 377.2[M + H]+. | 76 |

### Preparation of 9-methyl-6-(morpholin-4-yl)-2-(1-phenyl-1H-pyrazol-3-yl)-8-(pyridin-4-yl)-9H-purine (Compound 77)

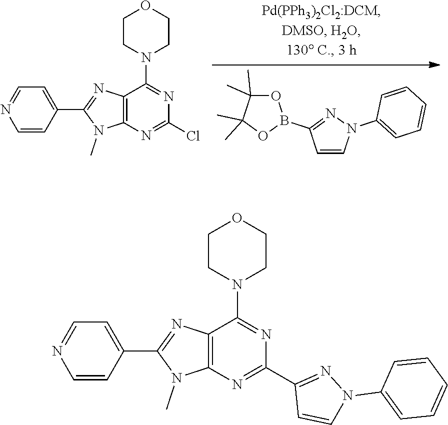

A mixture of 4-(2-chloro-9-methyl-8-(pyridin-4-yl)-9H-purin-6-yl)morpholine (100 mg, 0.30 mmol), 1-phenyl-3-(4,4,5,5-tetramethyl-1,3,2-dioxaborolan-2-yl)-1H-pyrazole (105 mg, 0.39 mmol), 1,1′-bis(diphenylphosphino)ferrocene palladium(II)dichloride dichloromethane complex (49.0 mg, 0.060 mmol) and cesium carbonate (293 mg, 0.90 mmol) in water (2 mL) and DMSO (8 mL) was stirred at 130° C. for 3 h under argon. The mixture was filtered over celite and washed with ethyl acetate (50 mL). The filtrate was further diluted with water (50 mL) and the layers were separated. The organic layer was washed with brine (20 mL), dried over sodium sulfate, filtered and concentrated under reduced pressure. The crude product was purified by prep-HPLC (Boston pHlex ODS 10 μm 21.2×250 mm 120 A. The mobile phase was acetonitrile/0.1% ammonium bicarbonate) to obtain 9-methyl-6-(morpholin-4-yl)-2-(1-phenyl-1H-pyrazol-3-yl)-8-(pyridin-4-yl)-9H-purine (64.5 mg, 0.15 mmol, 30%) as a yellow solid. ¹H NMR (400 MHz, Chloroform-d) δ 8.83-8.76 (m, 2H), 8.01 (d, J=2.5 Hz, 1H), 7.89-7.82 (m, 2H), 7.79-7.74 (m, 2H), 7.53-7.44 (m, 2H), 7.35-7.29 (m, 1H), 7.23 (d, J=2.5 Hz, 1H), 4.47 (s, 4H), 4.07 (s, 3H), 3.96-3.84 (m, 4H); LCMS (ESI) m/z: 439.2 [M+H]⁺.

The following compounds were synthesized according to the protocol described above.

| Name | Structure | 1H NMR Data | # |
|---|---|---|---|
| 4-(9-methyl-2- (1-(piperidin-3- yl)-1H-pyrazol- 3-yl)-8-(pyridin- 4-yl)-9H-purin- 6-yl)morpholine | 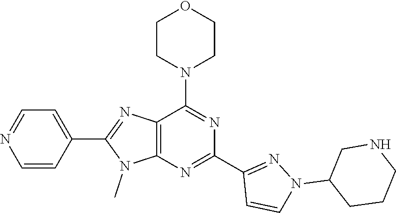 [SMILES: Cn1c(-c2ccncc2)nc2c(N3CCOCC3)nc(-c3ccn(C4CCCNC4)n3)nc21] | ¹H NMR (400 MHz, Dimethylsulfoxide-d₆) δ 8.80 (dd, J = 4.5, 1.6 Hz, 2H), 7.93 (dd, J = 4.5, 1.6 Hz, 2H), 7.53 (d, J = 1.8 Hz, 1H), 6.93 (d, J = 1.8 Hz, 1H), 5.58 (s, 1H), 4.32 (s, 4H), 3.96 (s, 3H), 3.86- 3.60 (m, 4H), 3.22 (m, 1H), 3.09-2.89 (m, 2H), 2.56 (m, 1H), 2.12 (m, 1H), 2.00 (m, 1H), 1.78 (m, 1H), 1.56 (m, 1H); LCMS (ESI) m/z: 446.3 [M + H]⁺. | 78 |
| tert-butyl 3-(5- (9-methyl-6- morpholino-8- (pyridin-4-yl)- 9H-purin-2-yl)- 1H-pyrazol-1- yl)piperidine-1- carboxylate | 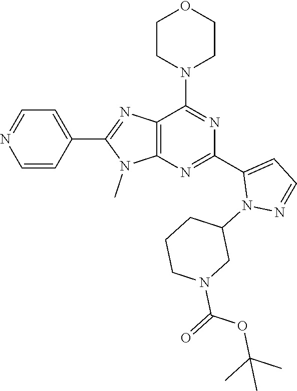 [SMILES: Cn1c(-c2ccncc2)nc2c(N3CCOCC3)nc(-c3ccnn3C3CCCN(C(=O)OC(C)(C)C)C3)nc21] | ¹H NMR (400 MHz, Dimethylsulfoxide-d₆) δ 8.78 (d, J = 6.0 Hz, 2H), 7.92 (d, J = 6.1 Hz, 2H), 7.88 (d, J = 2.3 Hz, 1H), 6.93 (d, J = 2.3 Hz, 1H), 4.24 (m, 7H), 3.96 (s, 3H), 3.86 (d, J = 13.1 Hz, 1H), 3.78 (d, J = 4.5 Hz, 4H), 2.92 (m, 1H), 2.14 (m, 1H), 2.06 (m, 1H), 1.81-1.74 (m, 1H), 1.53 (m, 1H), 1.42 (s, 9H); LCMS (ESI) m/z: 546.3 [M + H]⁺. | 79 |
| tert-butyl 3-(3- (9-methyl-6- morpholino-8- (pyridin-4-yl)- 9H-purin-2-yl)- 1H-pyrazol-1- yl)piperidine-1- carboxylate | 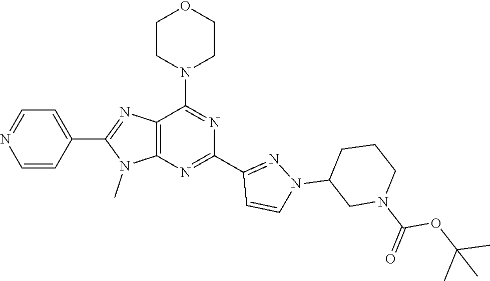 [SMILES: Cn1c(-c2ccncc2)nc2c(N3CCOCC3)nc(-c3ccn(C4CCCN(C(=O)OC(C)(C)C)C4)n3)nc21] | ¹H NMR (400 MHz, Dimethylsulfoxide-d₆) δ 8.79 (d, J = 5.8 Hz, 2H), 7.91 (d, J = 6.1 Hz, 2H), 7.56 (d, J = 1.8 Hz, 1H), 6.95 (d, J = 1.8 Hz, 1H), 5.62 (s, 1H), 4.28 (s, 4H), 3.97 (d, J = 2.8 Hz, 1H), 3.93 (s, 3H), 3.77 (t, J = 4.6 Hz, 4H), 2.86-2.77 (m, 1H), 2.12 (s, 2H), 1.85 (m, 1H), 1.40 (m, 11H); LCMS (ESI) m/z: 546.3 [M + H]⁺. | 80 |
| 4-(9-methyl-2- (1-methyl-1H- pyrazol-3-yl)-8- (pyridin-4-yl)- 9H-purin-6- yl)morpholine | 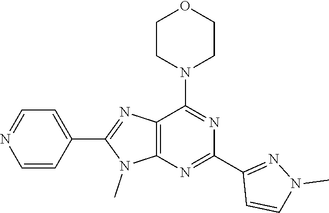 [SMILES: Cn1ccc(-c2nc(N3CCOCC3)c3nc(-c4ccncc4)n(C)c3n2)n1] | ¹H NMR (400 MHz, Dimethylsulfoxide-d₆) δ 8.79 (s, 2H), 7.91 (d, J = 4.8 Hz, 2H), 7.75 (s, 1H), 6.89 (s, 1H), 4.32 (s, 4H), 3.94 (d, J = 3.8 Hz, 6H), 3.77 (s, 4H); LCMS (ESI) m/z: 377.1 [M + H]⁺. | 81 |
| 4-(9-methyl-2- (1H-pyrazol-3- yl)-8-(pyridin-4- yl)-9H-purin-6- yl)morpholine | 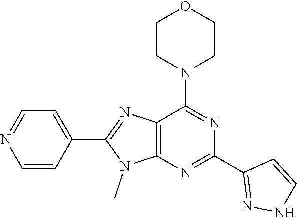 [SMILES: Cn1c(-c2ccncc2)nc2c(N3CCOCC3)nc(-c3cc[nH]n3)nc21] | ¹H NMR (400 MHz, Dimethylsulfoxide-d₆) δ 13.49 (s, 1H), 8.78 (d, J = 6.0 Hz, 2H), 7.92 (dd, J = 4.6, 1.5 Hz, 2H), 7.59 (s, 1H), 6.90 (d, J = 1.7 Hz, 1H), 4.37 (s, 4H), 3.97 (s, 3H), 3.82-3.69 (m, 4H); LCMS (ESI) m/z: 363.1 [M + H]⁺. | 82 |

### Synthesis of 4-(9-ethyl-2-(5-methyl-4,5,6,7-tetrahydro-1H-pyrazolo[4,3-c]pyridin-3-yl)-8-(pyridin-4-yl)-9H-purin-6-yl)morpholine (Compound 83)

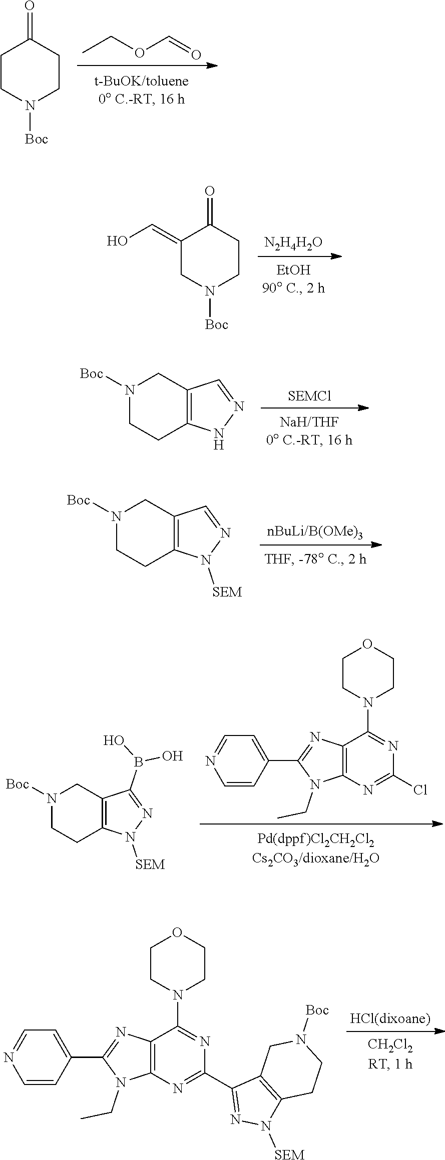

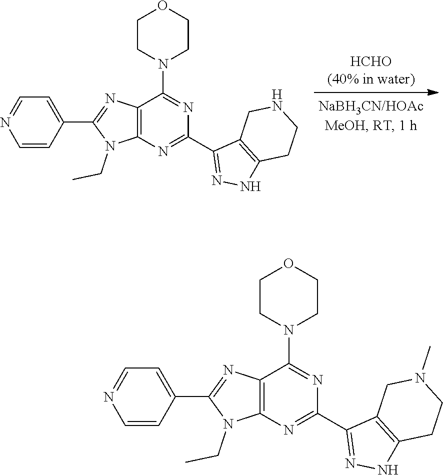 [SMILES: CCn1c(-c2ccncc2)nc2c(N3CCOCC3)nc(-c3n[nH]c4c3CNCC4)nc21 ; CCn1c(-c2ccncc2)nc2c(N3CCOCC3)nc(-c3n[nH]c4c3CN(C)CC4)nc21]

### Step 1: Synthesis of tert-butyl 3-(hydroxymethylene)-4-oxopiperidine-1-carboxylate

To a mixture of tert-butyl 4-oxopiperidine-1-carboxylate (700 mg, 3.5 mmol) in toluene (10 mL) was added potassium 2-methylpropan-2-olate (784 mg, 7.0 mmol) at 0° C. The mixture was stirred at 0° C. for 30 minutes, followed by the addition of ethyl formate (260 mg, 3.5 mmol). The resulting mixture was stirred for another 16 h, diluted with water and extracted with ethyl acetate (150 mL*2). The combined organic phase was dried and concentrated to afford tert-butyl 3-(hydroxymethylene)-4-oxopiperidine-1-carboxylate (700 mg, crude) as an orange oil. LCMS (ESI) m/z: 249.9.2 [M+Na]⁺.

### Step 2: Synthesis of tert-butyl 6,7-dihydro-1H-pyrazolo[4,3-c]pyridine-5(4H)-carboxylate

A mixture of tert-butyl 3-(hydroxymethylene)-4-oxopiperidine-1-carboxylate (600 mg, 1.0 mmol), hydrazine hydrate (98%, 1.0 mL) and ethanol (10 mL) was stirred at 90° C. for 2 h and then concentrated. The residue was purified by silica gel column chromatography (40% ethyl acetate in petroleum ether) to afford tert-butyl 6,7-dihydro-1H-pyrazolo[4,3-c]pyridine-5(4H)-carboxylate (200 mg, 0.9 mmol) as an off-white solid. LCMS (ESI) m/z: 223.9 [M+H]⁺.

### Step 3: Synthesis of tert-butyl 1-((2-(trimethylsilyl)ethoxy)methyl)-6,7-dihydro-1H-pyrazolo[4,3-c]pyridine-5(4H)-carboxylate

To a suspension of sodium hydride (72 mg, 1.8 mmol) in tetrahydrofuran (5 mL) was added a solution of tert-butyl 6,7-dihydro-1H-pyrazolo[4,3-c]pyridine-5(4H)-carboxylate (200 mg, 0.9 mmol) in tetrahydrofuran (5 mL) at 0° C. under nitrogen atmosphere. After stirring for 30 minutes, 2-(trimethylsilyl)ethoxymethyl chloride (166 mg, 1.0 mmol) was added thereto and stirred for another 2 h. The mixture was poured into crushed ice, extracted with ethyl acetate (100 mL*2). The combined organic phase was concentrated. The residue was purified by silica gel column chromatography (15% ethyl acetate in petroleum ether) to afford tert-butyl 1-((2-(trimethylsilyl)ethoxy)methyl)-6,7-dihydro-1H-pyrazolo[4,3-c]pyridine-5(4H)-carboxylate (200 mg, 62.2%) as pale yellow solid. LCMS (ESI) m/z: 354.0 [M+H]⁺.

### Step 4: Synthesis of 5-(tert-butoxycarbonyl)-1-((2-(trimethylsilyl)ethoxy)methyl)-4,5,6,7-tetrahydro-1H-pyrazolo[4,3-c]pyridin-3-ylboronic acid

To a solution of tert-butyl 1-((2-(trimethylsilyl)ethoxy)methyl)-6,7-dihydro-1H-pyrazolo[4,3-c]pyridine-5(4H)-carboxylate (200 mg, 0.56 mmol) in tetrahydrofuran (10 mL) at −78° C. was added dropwise a solution of butyllithium (2.5 mol/L in tetrahydrofuran, 0.5 mL) under nitrogen atmosphere. After the addition, the mixture was stirred at this temperature and stirred for another 30 minutes, followed by the addition of trimethyl borate (88 mg, 0.84 mmol). The resulting mixture was stirred at −78° C. for another 1 h. The reaction was quenched with ammonium chloride aqueous (30 mL) and extracted with ethyl acetate (100 mL*2). The combined organic phase was dried and concentrated to afford 5-(tert-butoxycarbonyl)-1-((2-(trimethylsilyl)ethoxy)methyl)-4,5,6,7-tetrahydro-1H-pyrazolo[4,3-c]pyridin-3-ylboronic acid (180 mg, crude) as a yellow oil. LCMS (ESI) m/z: 397.9 [M+H]⁺.

### Step 5: Synthesis of tert-butyl 3-(9-ethyl-6-morpholino-8-(pyridin-4-yl)-9H-purin-2-yl)-1-((2-(trimethylsilyl)ethoxy)methyl)-6,7-dihydro-1H-pyrazolo[4,3-c]pyridine-5(4H)-carboxylate

A mixture of 4-(2-chloro-9-ethyl-8-(pyridin-4-yl)-9H-purin-6-yl)morpholine (60 mg, 0.17 mmol), 5-(tert-butoxycarbonyl)-1-((2-(trimethylsilyl)ethoxy)methyl)-4,5,6,7-tetrahydro-1H-pyrazolo[4,3-c]pyridin-3-ylboronic acid (150 mg), 1,1′-Bis(diphenylphosphino)ferrocene-palladium(II)dichloride dichloromethane complex (28 mg, 0.034 mmol), water (0.5 mL) and dioxane (6 mL) was stirred at 100° C. under nitrogen atmosphere for 3 h. The mixture was poured into water and extracted with dichloromethane (100 mL*2). The combined organic phase was concentrated and the residue was purified by silica gel column chromatography (10% dichloromethane in methanol) to afford tert-butyl 3-(9-ethyl-6-morpholino-8-(pyridin-4-yl)-9H-purin-2-yl)-1-((2-(trimethylsilyl)ethoxy)methyl)-6,7-dihydro-1H-pyrazolo[4,3-c]pyridine-5(4H)-carboxylate (160 mg, 64% purity) as a light yellow oil. LCMS (ESI) m/z: 661.8 [M+H]⁺.

### Step 6: Synthesis of 4-(9-ethyl-8-(pyridin-4-yl)-2-(4,5,6,7-tetrahydro-1H-pyrazolo[4,3-c]pyridin-3-yl)-9H-purin-6-yl)morpholine

A mixture of tert-butyl 3-(9-ethyl-6-morpholino-8-(pyridin-4-yl)-9H-purin-2-yl)-1-((2-(trimethylsilyl)ethoxy)methyl)-6,7-dihydro-1H-pyrazolo[4,3-c]pyridine-5(4H)-carboxylate (100 mg), HCl (4 M in dixoane, 2 mL) and dichloromethane (10 mL) was stirred at 20° C. for 2 h. The mixture was quenched with aqueous saturated sodium bicarbonate and extracted with dichloromethane (50 mL*2). The organic phase was concentrated to afford 4-(9-ethyl-8-(pyridin-4-yl)-2-(4,5,6,7-tetrahydro-1H-pyrazolo[4,3-c]pyridin-3-yl)-9H-purin-6-yl) morpholine (50 mg,) as a light yellow solid. LCMS (ESI) m/z: 431.9 [M+H]⁺.

### Step 7: Synthesis of 4-(9-ethyl-2-(5-methyl-4,5,6,7-tetrahydro-1H-pyrazolo[4,3-c]pyridin-3-yl)-8-(pyridin-4-yl)-9H-purin-6-yl)morpholine

A mixture of 4-(9-ethyl-8-(pyridin-4-yl)-2-(4,5,6,7-tetrahydro-1H-pyrazolo[4,3-c]pyridin-3-yl)-9H-purin-6-yl)morpholine (40 mg), formaldehyde (40% in water, 2 mL), acetic acid (0.05 mL) and methanol (5 mL) was stirred at at 20° C. for 30 min, followed by the addition of sodium cyanoborohydride (63 mg, 1.0 mmol). The mixture was stirred at at 20° C. for another 30 min and concentrated. The crude product was purified by prep-HPLC (Column Xbridge 21.2*250 mm C18, 10 um, Mobile Phase A: water (10 mmol/L ammonium bicarbonate) B: acetonitrile) to afford 4-(9-ethyl-2-(5-methyl-4,5,6,7-tetrahydro-1H-pyrazolo[4,3-c]pyridin-3-yl)-8-(pyridin-4-yl)-9H-purin-6-yl)morpholine (13.1 mg, 0.029 mmol) as a white solid.

¹H NMR (400 MHz, CDCl₃) δ 8.81 (d, J=6.0 Hz, 2H), 7.69 (dd, J=4.5, 1.5 Hz, 2H), 4.50-4.30 (m, 6H), 3.98 (s, 2H), 3.93-3.81 (m, 4H), 2.95 (s, 2H), 2.87 (s, 2H), 2.60 (s, 3H), 1.50 (d, J=7.2 Hz, 3H); LCMS (ESI) m/z: 445.8 [M+H]⁺.

### Synthesis of 4-(2-(1-cyclopropyl-1H-pyrazol-3-yl)-9-ethyl-8-(pyridin-4-yl)-9H-purin-6-yl)morpholine (Compound 84) and 4-(2-(1-cyclopropyl-1H-pyrazol-5-yl)-9-ethyl-8-(pyridin-4-yl)-9H-purin-6-yl)morpholine (Compound 85)

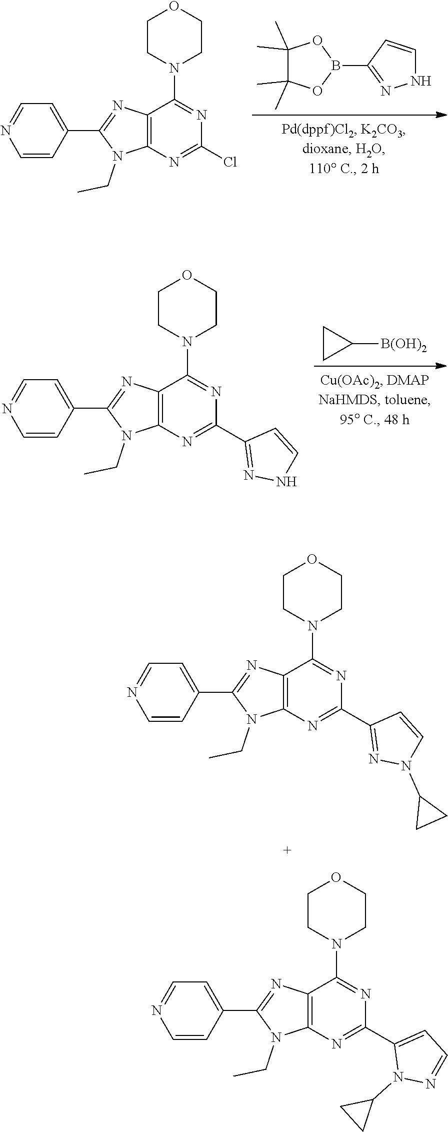 [SMILES: CCn1c(-c2ccncc2)nc2c(N3CCOCC3)nc(Cl)nc21 ; CC1(C)OB(c2cc[nH]n2)OC1(C)C ; CCn1c(-c2ccncc2)nc2c(N3CCOCC3)nc(-c3cc[nH]n3)nc21 ; OB(O)C1CC1 ; CCn1c(-c2ccncc2)nc2c(N3CCOCC3)nc(-c3ccn(C4CC4)n3)nc21 ; CCn1c(-c2ccncc2)nc2c(N3CCOCC3)nc(-c3ccnn3C3CC3)nc21]

### Step 1: Preparation of 4-(9-ethyl-2-(1H-pyrazol-3-yl)-8-(pyridin-4-yl)-9H-purin-6-yl)morpholine

To a solution of 4-(2-chloro-9-ethyl-8-(pyridin-4-yl)-9H-purin-6-yl)morpholine (300 mg, 0.9 mmol) in dioxane (5 mL) and water (1 mL) were added 3-(4,4,5,5-tetramethyl-1,3,2-dioxaborolan-2-yl)-1H-pyrazole (262 mg, 1.35 mmol), [1,1′-bis(diphenylphosphino) ferrocene]dichloropalladium(II) (65.8 mg, 0.09 mmol) and potassium carbonate (373 mg, 2.7 mmol) at 25° C. and the reaction mixture was stirred at 110° C. for 2 h under N₂ protection. The mixture was extracted with dichloromethane (20 mL*2) and washed with water (10 mL*2). The organic layer was dried over sodium sulfate, and concentrated. The residue was purified by silica gel column chromatography (5% methanol in dichloromethane) to obtain 4-(9-ethyl-2-(1H-pyrazol-3-yl)-8-(pyridin-4-yl)-9H-purin-6-yl)morpholine as white solid (290 mg, 73.8%). LCMS (ESI) m/z: 377.0 [M+H]⁺.

### Step 2: Preparation of 4-(2-(1-cyclopropyl-1H-pyrazol-3-yl)-9-ethyl-8-(pyridin-4-yl)-9H-purin-6-yl)morpholine

To a solution of 4-(9-ethyl-2-(1H-pyrazol-3-yl)-8-(pyridin-4-yl)-9H-purin-6-yl)morpholine (200.0 mg, 0.52 mmol) in toluene (15 mL) was added cyclopropylboronic acid (91.4 mg, 1.06 mmol), cupric acetate (99.9 mg, 0.52 mmol), DMAP (194.9 mg, 1.59 mmol) and sodium bis(trimethylsilyl)amide (0.53 mL) at 25° C., and the reaction mixture was heated to 95° C. and stirred for 48 h under N₂ protection. The mixture was extracted with dichloromethane (20 mL*2) and washed with water (10 mL*2). The organic layer was dried over sodium sulfate, and concentrated. The residue was purified with prep-HPLC (BOSTON pHlex ODS 10 um 21.2×250 mm 120 A. The mobile phase was DMSO/0.1% Ammonium bicarbonate) to obtain 4-(2-(1-cyclopropyl-1H-pyrazol-3-yl)-9-ethyl-8-(pyridin-4-yl)-9H-purin-6-yl)morpholine as white solid (22.3 mg, 20.6%) and 4-(2-(1-cyclopropyl-1H-pyrazol-5-yl)-9-ethyl-8-(pyridin-4-yl)-9H-purin-6-yl)morpholine as white solid (81.1 mg, 75.1%).

Compound 84: ¹H NMR (400 MHz, DMSO-d₆) δ 8.80 (s, 2H), 7.85 (d, J=5.4 Hz, 2H), 7.80 (d, J=2.2 Hz, 1H), 6.87 (d, J=2.2 Hz, 1H), 4.43 (q, J=7.2 Hz, 2H), 4.30 (bs, 4H), 3.80-3.75 (m, 5H), 1.32 (t, J=7.2 Hz, 3H), 1.15-1.07 (m, 2H), 1.07-0.98 (m, 2H); LCMS (ESI) m/z: 417.0 [M+H]⁺.

Compound 85: ¹H NMR (400 MHz, DMSO-d₆) δ 8.81 (d, J=5.4 Hz, 2H), 7.85 (d, J=6.0 Hz, 2H), 7.43 (d, J=1.8 Hz, 1H), 6.92 (d, J=1.8 Hz, 1H), 4.69-4.62 (m, 1H), 4.42 (q, J=7.1 Hz, 2), 4.39 (bs, 4H), 3.83-3.72 (m, 4H), 1.37 (t, J=7.1 Hz, 3H), 1.16-1.10 (m, 2H), 1.05-0.97 (m, 2H); LCMS (ESI) m/z: 417.0 [M+H]⁺.

### Synthesis of 4-(9-ethyl-2-(3-phenyl-1H-pyrazol-5-yl)-8-(pyridin-4-yl)-9H-purin-6-yl)morpholine (Compound 86)

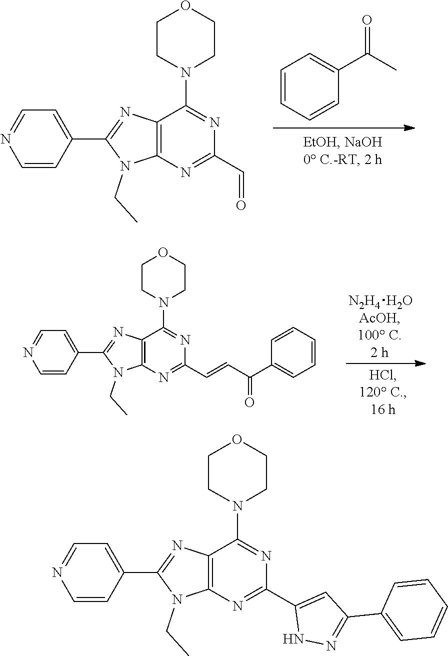

### Step 1: Preparation of (E)-3-(9-ethyl-6-morpholino-8-(pyridin-4-yl)-9H-purin-2-yl)-1-phenylprop-2-en-1-one

To a solution of 9-ethyl-6-morpholino-8-(pyridin-4-yl)-9H-purine-2-carbaldehyde (150.0 mg, 0.45 mmol) in ethanol (20 ml) was added acetophenone (44.3 mg, 0.37 mmol) under ice-bath cooling and slowly warmed up to 24° C. and stirred for 2.0 h. The resultant mixture was extracted with ethyl acetate (20 mL*2) and washed with water (10 mL*2). The organic layer was dried over sodium sulfate, and concentrated. The residue was purified by silica gel column chromatography (5% methanol in dichloromethane) to obtain (E)-3-(9-ethyl-6-morpholino-8-(pyridin-4-yl)-9H-purin-2-yl)-1-phenylprop-2-en-1-one as white solid (100 mg, 61.3%). LCMS (ESI) m/z: 441.8 [M+H]⁺.

### Step 2: Preparation of 4-(9-ethyl-2-(3-phenyl-1H-pyrazol-5-yl)-8-(pyridin-4-yl)-9H-purin-6-yl)morpholine

A mixture of (E)-3-(9-ethyl-6-morpholino-8-(pyridin-4-yl)-9H-purin-2-yl)-1-phenylprop-2-en-1-one (135.0 mg, 0.3 mmol) and hydrazine hydrate (46.1 mg, 0.9 mol) in acetic acid (20 mL) was stirred at reflux for 2 h. Then hydrochloric acid (20 mL) was added and stirred at reflux for 16 h. The resultant mixture was extracted with ethyl acetate (20 mL*2), dried over sodium sulfate, and concentrated. The residue was purified by silica gel column chromatography (5% methanol in dichloromethane) to obtain 4-(9-ethyl-2-(3-phenyl-1H-pyrazol-5-yl)-8-(pyridin-4-yl)-9H-purin-6-yl)morpholine as white solid (15.5 mg, 11.4%).

¹H NMR (500 MHz, DMSO-d₆) δ 13.55 (s, 1H), 8.80 (s, 2H), 7.93 (d, J=7.7 Hz, 2H), 7.87 (d, J=5.3 Hz, 2H), 7.44 (t, J=7.6 Hz, 2H), 7.36-7.30 (m, 2H), 4.48 (q, J=7.2 Hz, 2H), 4.46 (bs, 4H), 3.83-3.74 (m, 4H), 1.36 (t, J=7.2 Hz, 3H); LCMS (ESI) m/z: 453.0 [M+H]⁺.

### Synthesis of 4-(9-ethyl-8-(pyridin-4-yl)-2-(4,5,6,7-tetrahydropyrazolo[1,5-a]pyrazin-3-yl)-9H-purin-6-yl)morpholine (Compound 87)

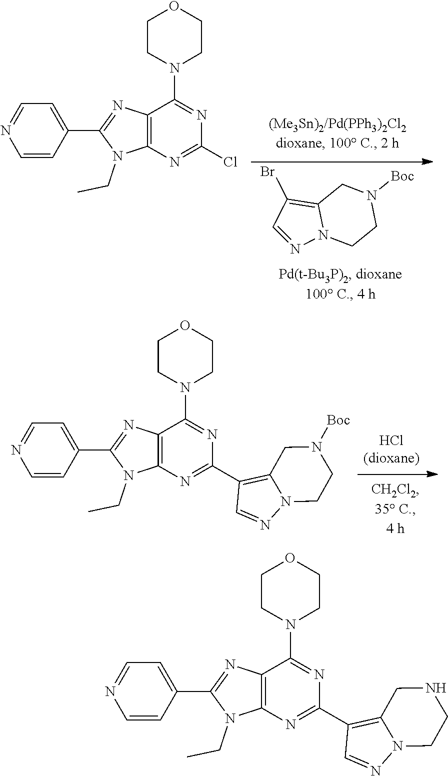

### Step 1: Synthesis of tert-butyl 3-(9-ethyl-6-morpholino-8-(pyridin-4-yl)-9H-purin-2-yl)-6,7-dihydropyrazolo[1,5-a]pyrazine-5(4H)-carboxylate

A mixture of 4-(2-chloro-9-ethyl-8-(pyridin-4-yl)-9H-purin-6-yl)morpholine (34 mg, 0.1 mmol), 1,1,1,2,2,2-hexamethyldistannane (65 mg, 0.2 mmol) and bis(triphenylphosphine)palladium(II) chloride (14 mg, 0.02 mmol) in dioxane (2 mL) was stirred at 100° C. for 2 h. The mixture was cooled and tert-butyl 3-bromo-6,7-dihydropyrazolo[1,5-a]pyrazine-5(4H)-carboxylate (30 mg, 0.1 mmol), bis(tri-tert-butylphosphine)palladium (11 mg, 0.02 mmol) were added to the reaction mixture. The mixture was stirred for another 4 h and concentrated. The resultant residue was purified by silica gel column chromatography (15% methanol in dichloromethane) and prep-HPLC (Column Xbridge 21.2*250 mm C18, 10 um, Mobile Phase A: water (10 mmol/L ammonium bicarbonate) B: acetonitrile) to afford tert-butyl 3-(9-ethyl-6-morpholino-8-(pyridin-4-yl)-9H-purin-2-yl)-6,7-dihydropyrazolo[1,5-a]pyrazine-5(4H)-carboxylate (25 mg, 9.4%) as a white solid. LCMS (ESI) m/z: 531.8 [M+H]⁺.

### Step 2: Synthesis of 4-(9-ethyl-8-(pyridin-4-yl)-2-(4,5,6,7-tetrahydropyrazolo[1,5-a]pyrazin-3-yl)-9H-purin-6-yl)morpholine

A mixture of tert-butyl 3-(9-ethyl-6-morpholino-8-(pyridin-4-yl)-9H-purin-2-yl)-6,7-dihydropyrazolo[1,5-a]pyrazine-5(4H)-carboxylate (20 mg, 0.037 mmol) and hydrochloric acid (4 M in dixoane, 2 mL) in dichloromethane (5 mL) was stirred at 30° C. for 2 h. The mixture was quenched with ammonium in methanol (7.0 M, 10 mL) and concentrated. The residue was purified by silica gel column chromatography (30% dichloromethane in methanol) and prep-HPLC (Column Xbridge 21.2*250 mm C18, 10 um, Mobile Phase A: water (10 mmol/L ammonium bicarbonate) B: acetonitrile) to afford 4-(9-ethyl-8-(pyridin-4-yl)-2-(4,5,6,7-tetrahydropyrazolo[1,5-a]pyrazin-3-yl)-9H-purin-6-yl)morpholine (9.1 mg, 56.7%) as a white solid.

¹H NMR (400 MHz, CDCl₃) δ 8.79 (s, 2H), 8.18 (s, 1H), 7.69 (d, J=5.8 Hz, 2H), 4.58 (s, 2H), 4.45-4.25 (m, 6H), 4.20 (t, J=5.4 Hz, 2H), 3.91-3.83 (m, 4H), 3.36 (s, 2H), 1.49 (t, J=7.2 Hz, 3H); LCMS (ESI) m/z: 431.9 [M+H]⁺.

### Synthesis of 4-(9-methyl-2-(4-methyl-1-phenyl-1H-pyrazol-3-yl)-8-(pyridin-4-yl)-9H-purin-6-yl)morpholine (Compound 88)

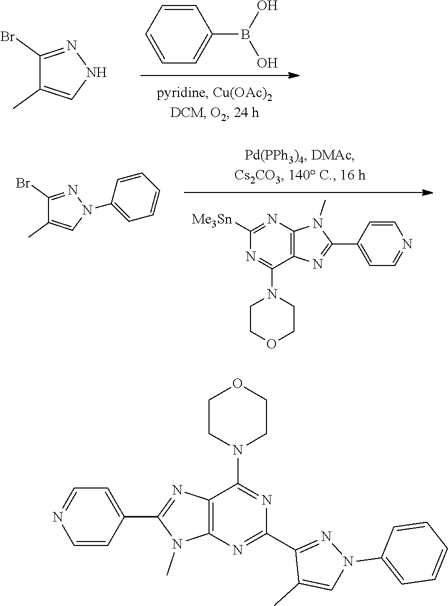

### Step 1: 3-bromo-4-methyl-1-phenyl-1H-pyrazole

A mixture of phenylboronic acid (300 mg, 2.48 mmol), pyridine (300 mg, 3.73 mmol), copper acetate (500 mg, 2.48 mmol) and 3-bromo-4-methyl-1H-pyrazole (200 mg, 0.61 mmol) in dichloromethane (10 mL) was stirred at 45° C. under oxygen for 24 h. The reaction mixture was diluted with water (30 mL) and the resulting mixture was extracted with dichloromethane (30 mL×3). The combined organic layers were washed with brine (20 mL), dried over anhydrous sodium sulfate and concentrated in vacuo. The residue was purified by silica gel column chromatography eluting with Petroleum ether/Ethyl acetate (v/v) 5/1 to obtain the desired product as yellow liquid (120 mg, 41%). LCMS (ESI) m/z: 238.1/239.0 [M+H]+.

### Step 2: 4-(9-methyl-2-(4-methyl-1-phenyl-1H-pyrazol-3-yl)-8-(pyridin-4-yl)-9H-purin-6-yl)morpholine

To a solution of 4-(9-methyl-8-(pyridin-4-yl)-2-(trimethylstannyl)-9H-purin-6-yl)morpholine (30 mg, 0.06 mmol) in dioxane (5 mL) was added 3-bromo-4-methyl-1-phenyl-1H-pyrazole (16 mg, 0.06 mmol), tetrakis(triphenylphosphine)palladium (1 mg, 0.006 mmol) at 25° C. and the reaction mixture was stirred at 100° C. for 17 h under nitrogen atmosphere. The resultant mixture was extracted with ethyl acetate (20 mL*2) and washed with water (10 mL*2), dried and concentrated. The crude product was purified by flash chromatography on silica gel (dichloromethane/methanol 10:1) to obtain the desired product (5 mg, 18.5%).

1H NMR (400 MHz, CDCl3) δ 8.88 (d, J=6.2 Hz, 2H), 8.14 (d, J=6.4 Hz, 2H), 7.87-7.76 (m, 3H), 7.48 (dd, J=18.3, 10.6 Hz, 2H), 7.31 (d, J=7.4 Hz, 1H), 4.47 (s, 4H), 4.15 (s, 3H), 3.98-3.85 (m, 4H), 2.55 (s, 3H); LCMS (ESI) m/z: 453.7 [M+H]+.

The following compound was synthesized according to the protocol described above:

| Name | Structure | NMR, MS | # |
|---|---|---|---|
| 4-(9-methyl-2-(2- methylpyrimidin- 4-yl)-8-(pyridin-4- yl)-9H-purin-6- yl)morpholine | 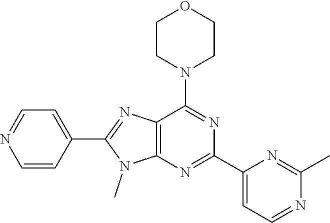 [SMILES: Cc1nccc(-c2nc(N3CCOCC3)c3nc(-c4ccncc4)n(C)c3n2)n1] | ¹H NMR (400 MHz, CDCl₃) δ 8.81 (t, J = 5.6 Hz, 3H), 8.20 (d, J = 5.1 Hz, 1H), 7.78 (d, J = 6.1 Hz, 2H), 4.46 (m, 4H), 4.10 (s, 3H), 3.92-3.90 (m, 4H), 2.91 (s, 3H); LCMS (ESI) m/z: 389.1 [M + H]⁺. | 89 |

### Synthesis of 1-methyl-4-(3-(9-methyl-6-morpholino-8-(pyridin-4-yl)-9H-purin-2-yl)-1H-pyrazol-1-yl)pyridin-2(1H)-one (Compound 90) and 1-methyl-4-(5-(9-methyl-6-morpholino-8-(pyridin-4-yl)-9H-purin-2-yl)-1H-pyrazol-1-yl)pyridin-2(1H)-one (Compound 91)

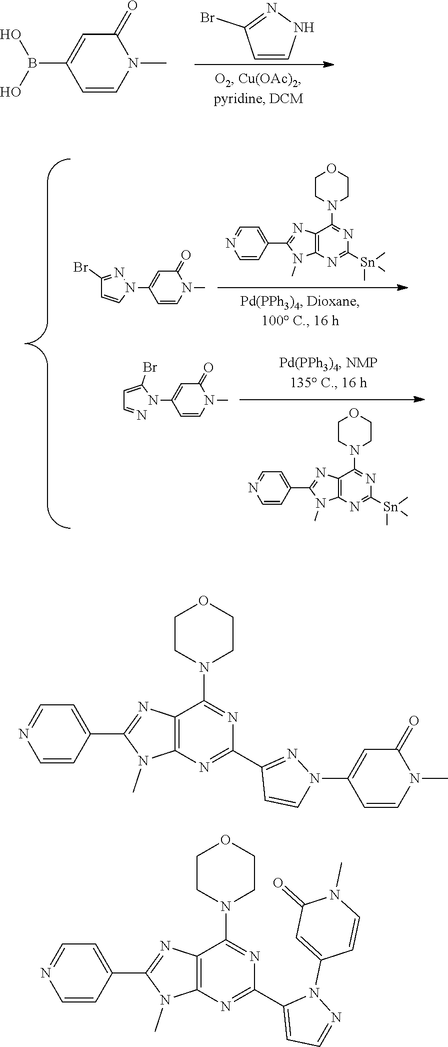

### Step 1: Synthesis of 4-(5-bromo-1H-pyrazol-1-yl)-1-methylpyridin-2(1H)-one (Step 1PA) and 4-(3-bromo-1H-pyrazol-1-yl)-1-methylpyridin-2(1H)-one (Step 1PB)

To a solution of 1-methyl-2-oxo-1,2-dihydropyridin-4-ylboronic acid (400 mg, 2.6 mmol), 3-bromo-1H-pyrazole (382 mg, 2.6 mmol) in dichloromethane (10 mL) were added cupric acetate (946 mg, 5.2 mmol) and pyridine (616 mg, 7.8 mmol). The reaction mixture was stirred at 45° C. for 16 h under oxygen. The reaction mixture was concentrated and purified by pre-HPLC (BOSTON pHlex ODS 10 μm 21.2×250 mm 120 A. The mobile phase was acetonitrile/0.1% Ammonium bicarbonate) to afford two products as green solids: 4-(5-bromo-1H-pyrazol-1-yl)-1-methylpyridin-2(1H)-one (50 mg, 7%) and 4-(3-bromo-1H-pyrazol-1-yl)-1-methylpyridin-2(1H)-one (200 mg, 28%) were isolated.

Step 1PA: 1H NMR (400 MHz, DMSO-d₆) δ 7.89 (d, J=7.3 Hz, 1H), 7.86 (d, J=1.7 Hz, 1H), 6.78 (d, J=1.8 Hz, 1H), 6.65 (d, J=2.3 Hz, 1H), 6.57 (dd, J=7.3, 2.4 Hz, 1H), 3.48 (s, 3H); LCMS: [M+H]+=254.

Step 1 PB: 1H NMR (400 MHz, DMSO-d₆) δ 8.64 (d, J=2.6 Hz, 1H), 7.87 (d, J=8.0 Hz, 1H), 6.80 (dd, J=5.5, 2.8 Hz, 3H), 3.44 (s, 3H); LCMS: [M+H]+=254.

Step 2: Synthesis of 1-methyl-4-(3-(9-methyl-6-morpholino-8-(pyridin-4-yl)-9H-purin-2-yl)-1H-pyrazol-1-yl)pyridin-2(1H)-one (Compound 90).

To a solution of 4-(3-bromo-1H-pyrazol-1-yl)-1-methylpyridin-2(1H)-one (80 mg, 0.315 mmol) and 4-(9-methyl-8-(pyridin-4-yl)-2-(trimethylstannyl)-9H-purin-6-yl)morpholine (217 mg, 0.472 mmol) in dioxane (5 mL) was added Pd(PPh3)4 (36 mg, 0.0315 mmol) and the resultant mixture was stirred at 100° C. for 16 h under argon. The crude product formed was purified by Prep-HPLC (BOSTON pHlex ODS 10 μm 21.2×250 mm120 A. The mobile phase was acetonitrile/0.1% Formic acid) to give 1-methyl-4-(3-(9-methyl-6-morpholino-8-(pyridin-4-yl)-9H-purin-2-yl)-1H-pyrazol-1-yl)pyridin-2(1H)-one as white solid. (12.4 mg, 6.4%). 1H NMR (500 MHz, DMSO-d₆) δ 8.80 (d, J=3.6 Hz, 2H), 8.70 (d, J=2.6 Hz, 1H), 7.93 (d, J=5.7 Hz, 2H), 7.90 (d, J=7.5 Hz, 1H), 7.24 (d, J=2.6 Hz, 1H), 6.98 (dd, J=7.3, 2.4 Hz, 1H), 6.94 (d, J=2.3 Hz, 1H), 4.36 (s, 4H), 3.99 (s, 3H), 3.84-3.74 (m, 4H), 3.47 (s, 3H); LCMS: [M+H]+=470.1.

### Step 3: Synthesis of 1-methyl-4-(5-(9-methyl-6-morpholino-8-(pyridin-4-yl)-9H-purin-2-yl)-1H-pyrazol-1-yl)pyridin-2(1H)-one (Compound 91)

To a solution of 4-(5-bromo-1H-pyrazol-1-yl)-1-methylpyridin-2(1H)-one (40 mg, 0.157 mmol) and 4-(9-methyl-8-(pyridin-4-yl)-2-(trimethylstannyl)-9H-purin-6-yl)morpholine (109 mg, 0.236 mmol) in dry NMP (4 mL) was added Pd(PPh3)4 (18 mg, 0.0157 mmol) and the resultant mixture was stirred at 135° C. for 16 h under argon. It was concentrated and product was purified by Prep-HPLC (BOSTON pHlex ODS 10 μm 21.2×250 mm120 A. The mobile phase was acetonitrile/0.1% Formic acid) to give 1-methyl-4-(5-(9-methyl-6-morpholino-8-(pyridin-4-yl)-9H-purin-2-yl)-1H-pyrazol-1-yl)pyridin-2(1H)-one as white solid (4.3 mg, 5%). 1H NMR (500 MHz, DMSO-d₆) δ 8.79 (d, J=5.7 Hz, 2H), 7.90 (d, J=6.0 Hz, 2H), 7.82 (d, J=1.7 Hz, 1H), 7.75 (d, J=7.2 Hz, 1H), 7.07 (d, J=1.6 Hz, 1H), 6.31 (d, J=2.2 Hz, 1H), 6.25 (dd, J=7.2, 2.3 Hz, 1H), 3.89 (s, 3H), 3.62 (s, 4H), 3.46 (s, 3H); LCMS [M+H]+=470.1.

### Synthesis of 4-(9-ethyl-8-(pyridin-4-yl)-2-(2-(tetrahydro-2H-pyran-4-yl)pyrimidin-4-yl)-9H-purin-6-yl)morpholine (Compound 92)

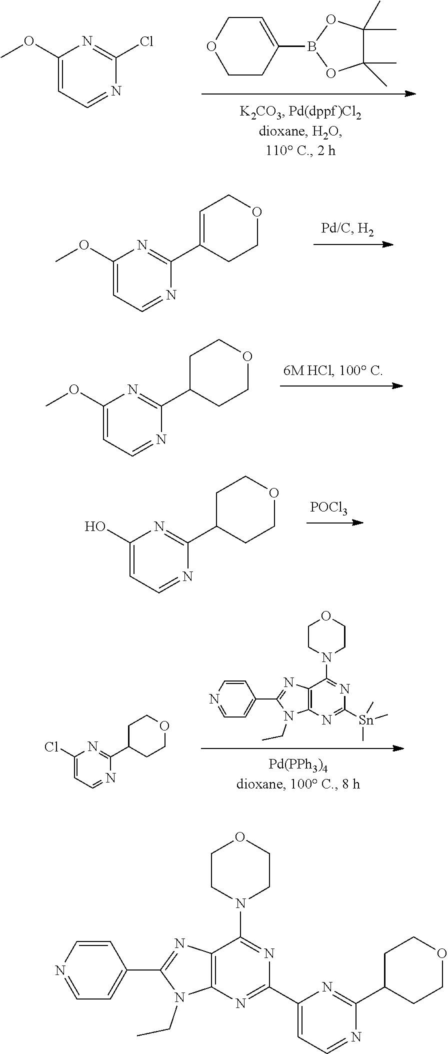

### Step 1: Preparation of 2-(3,6-dihydro-2H-pyran-4-yl)-4-methoxypyrimidine

To a solution of 2-chloro-4-methoxypyrimidine (870 mg, 6.041 mmol) in dioxane (10 mL) and water (5 mL) were added 2-(3,6-dihydro-2H-pyran-4-yl)-4,4,5,5-tetramethyl-1,3,2-dioxaborolane (1.5 g, 7.25 mmol), potassium carbonate (1.6 g, 12.08 mmol) and dichloro[1,1′-bis(diphenylphosphino)ferrocene]palladium(II) (70 mg, 0.10 mmol). The resultant mixture was stirred at 110° C. for 2.0 h. It was then cooled and concentrated. The residue was purified by flash chromatography on silica gel (petroleum ether:ethyl acetate=5:95) to give the desired product as yellow solid (860 mg, 74.2%).

### Step 2: Preparation of 4-methoxy-2-(tetrahydro-2H-pyran-4-yl)pyrimidine

A solution of 2-(3,6-dihydro-2H-pyran-4-yl)-4-methoxypyrimidine (860 mg, 4.42 mmol), palladium (10% on carbon, 30 mg) in methanol (10 mL) was stirred at 30° C. for 2.5 h under hydrogen atmosphere. The mixture was filtered and concentrated. The residue was purified by flash chromatography on silica gel (petroleum ether:ethyl acetate=75:25) to give the desired product as yellow oil (750 mg, 87.5%).

### Step 3: Preparation of 2-(tetrahydro-2H-pyran-4-yl)pyrimidin-4-ol

To a solution of 4-methoxy-2-(tetrahydro-2H-pyran-4-yl)pyrimidine (750 mg, 3.86 mmol) in water (10 mL) was added hydrochloric acid (6 M, 10 mL). The reaction mixture was stirred at 100° C. for 3 h and concentrated. The residue was diluted with water (20 mL), then adjusted the pH with NaHCO₃ to about 4 and the aqueous phase was extracted with ethyl acetate (20 ml×3). The organic layer was washed with water (20 mL) and brine (20 mL), dried over Na₂SO₄ and concentrated to give the target compound as brown solid (500 mg).

### Step 4: Preparation of 4-chloro-2-(tetrahydro-2H-pyran-4-yl)pyrimidine

A mixture of 2-(tetrahydro-2H-pyran-4-yl)pyrimidin-4-ol (500 mg, 2.78 mmol) and phosphoryl trichloride (10 mL) was stirred at 80° C. for 3 h. The mixture was concentrated and diluted with water (20 mL), then adjusted the pH with NaHCO₃ (2M) to about 7 and the aqueous phase was extracted with ethyl acetate (20 ml×3). The organic layer was washed with water (20 mL) and brine (20 mL), dried over sodium sulfate, filtered and concentrated. The residue was purified by flash chromatography on silica gel (petroleum ether:ethyl acetate=75:25) to obtain the desired product as white solid (550 mg, 100%).

### Step 5: Preparation of 4-(9-ethyl-8-(pyridin-4-yl)-2-(2-(tetrahydro-2H-pyran-4-yl)pyrimidin-4-yl)-9H-purin-6-yl)morpholine

To a solution of 4-(9-ethyl-8-(pyridin-4-yl)-2-(trimethylstannyl)-9H-purin-6-yl)morpholine (150 mg) in dioxane (10 mL) were added 4-chloro-2-(tetrahydro-2H-pyran-4-yl)pyrimidine (200 mg, 1.01 mmol, 1.0 e.q.) and tetrakis(triphenylphosphine)palladium (58 mg, 0.05 mmol). The mixture was stirred at 100° C. for 16 h. It was concentrated and the crude product thus obtained was purified by Prep-HPLC (BOSTON pHlex ODS 10 μm 21.2×250 mm 120 A. The mobile phase was acetonitrile/0.1% Ammonium bicarbonate) to obtain 4-(9-ethyl-8-(pyridin-4-yl)-2-(2-(tetrahydro-2H-pyran-4-yl)pyrimidin-4-yl)-9H-purin-6-yl)morpholine (32 mg, 6.7%) as white solid. 1H NMR (400 MHz, DMSO-d₆) δ 8.92 (d, J=5.1 Hz, 1H), 8.82 (d, J=6.0 Hz, 2H), 8.21 (d, J=5.1 Hz, 1H), 7.87 (dd, J=4.5, 1.6 Hz, 2H), 4.49 (q, J=7.2 Hz, 2H), 4.37 (bs, 4H), 3.98 (d, J=11.2 Hz, 2H), 3.87-3.77 (m, 4H), 3.52 (td, J=11.3, 3.0 Hz, 2H), 3.20 (dt, J=9.6, 5.5 Hz, 1H), 2.00-1.86 (m, 4H), 1.36 (t, J=7.2 Hz, 3H); LCMS (ESI) m/z: 472.8[M+H]+.

### Synthesis of 9-phenyl-2,6-di(pyridin-4-yl)-9H-purine (Compound 93)

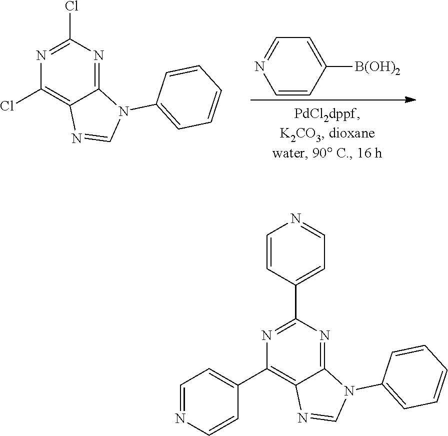

To a solution of 2,6-dichloro-9-phenyl-9H-purine (264 mg, 1 mmol) in dioxane (10 mL) and water (2 mL) were added pyridin-4-ylboronic acid (123 mg, 1 mmol) and [1,1′-bis(diphenylphosphino)ferrocene]dichloropalladium(II) (81 mg, 0.1 mmol) and potassium carbonate (414 mg, 3 mmol) at 25° C. and the resultant mixture was stirred at 90° C. for 16 h under argon protection. It was then extracted with ethyl acetate (20 mL*3) and washed with water (20 mL). The organic layer was dried over sodium sulfate, concentrated and purified by prep-HPLC (BOSTON pHlex ODS 10 μm 21.2×250 mm120 A. The mobile phase was acetonitrile/0.1% Ammonium bicarbonate) to give 9-phenyl-2,6-di(pyridin-4-yl)-9H-purine (13 mg, 4%) as a yellow solid. (2-chloro-9-phenyl-6-(pyridin-4-yl)-9H-purine was also isolated as the major product).

¹H NMR (400 MHz, DMSO-d₆) δ 9.29 (s, 1H), 8.88-8.94 (m, 4H), 8.81 (d, J=6.0 Hz, 2H), 8.46 (d, J=6.0 Hz, 2H), 8.06 (d, J=7.6 Hz, 2H), 7.73 (t, J=7.6 Hz, 2H), 7.60 (t, J=7.6 Hz, 1H); LCMS (ESI) m/z: 351.1 [M+H]+.

### Synthesis of 9-methyl-6-(morpholin-4-yl)-2-[3-(pyridin-3-yl)-1H-pyrazol-1-yl]-8-(pyridin-4-yl)-9H-purine (Compound 94)

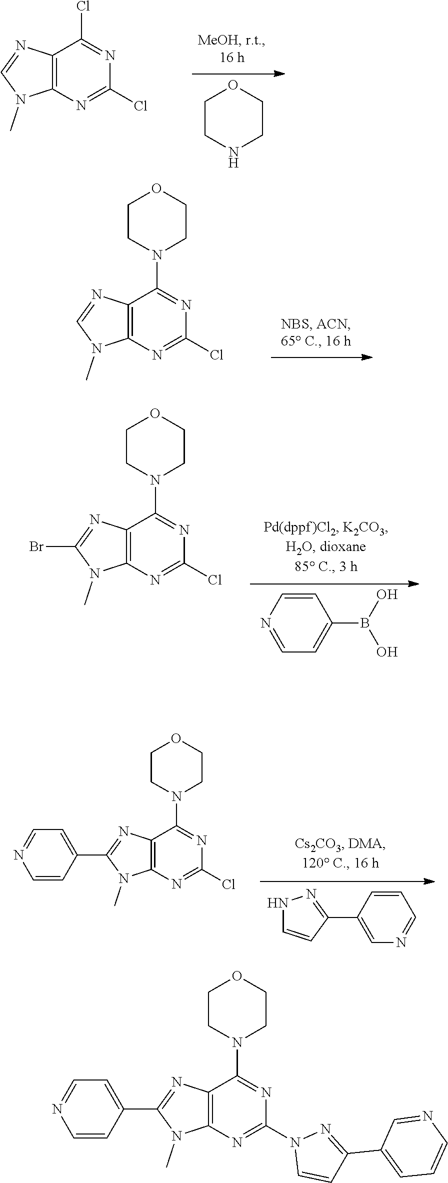

### Step 1: Preparation of 4-(2-Chloro-9-methyl-9H-purin-6-yl)morpholine

A mixture of 2,6-dichloro-9-methyl-9H-purine (6.00 g, 30 mmol) and morpholine (6.50 g, 74 mmol) in methanol (300 mL) was stirred at room temperature for 16 h. The mixture was filtered and the residue was triturated with methanol. The product 4-(2-Chloro-9-methyl-9H-purin-6-yl)morpholine (7.00 g, 28 mmol, 93%) was obtained as a white solid and carried onto next step without further purification. LCMS (ESI) m/z: 254.1 [M+H]⁺.

### Step 2: Preparation of 4-(8-Bromo-2-chloro-9-methyl-9H-purin-6-yl)morpholine

A mixture of 4-(2-chloro-9-methyl-9H-purin-6-yl)morpholine (7.00 g, 28 mmol) and N-bromosuccinimide (8.80 g, 50 mmol) in acetonitrile (500 mL) was stirred at 65° C. for 16 h. The mixture was filtered and the residue was triturated with acetonitrile. The product 4-(8-Bromo-2-chloro-9-methyl-9H-purin-6-yl)morpholine (8.00 g, 24 mmol, 87%) was isolated as light yellow solid and carried onto next step without further purification. LCMS (ESI) m/z: 332.3 [M+H]⁺.

### Step 3: Preparation of 4-(2-Chloro-9-methyl-8-(pyridin-4-yl)-9H-purin-6-yl)morpholine

A mixture of 4-(8-bromo-2-chloro-9-methyl-9H-purin-6-yl)morpholine (5.00 mmol), pyridin-4-ylboronic acid (2.20 g, 18 mmol), 1,1′-bis(diphenylphosphino)ferrocene-palladium(II)dichloride (1.10 g, 1.5 mmol) and potassium carbonate (5.20 g, 38 mmol) in dioxane (50 mL) and water (5 mL) under nitrogen was stirred at 85° C. for 3 h. The reaction mixture was filtered over celite and washed with ethyl acetate (3×25 mL). The filtrate was concentrated under reduced pressure and the resultant crude product was purified via flash column chromatography through silica gel using a gradient of 0-5% methanol in dichloromethane to obtain 4-(2-Chloro-9-methyl-8-(pyridin-4-yl)-9H-purin-6-yl)morpholine (3.00 g, 9.1 mmol, 60%) as a light-yellow solid. LCMS (ESI) m/z: 331.1 [M+H]⁺.

### Step 4: Preparation of 4-(9-methyl-2-(3-(pyridin-3-yl)-1H-pyrazol-1-yl)-8-(pyridin-4-yl)-9H-purin-6-yl)morpholine

A mixture of 4-(2-chloro-9-methyl-8-(pyridin-4-yl)-9H-purin-6-yl)morpholine (100 mg, 0.30 mmol), 3-(1H-pyrazol-3-yl)pyridine (58.0 mg, 0.40 mmol) and cesium carbonate (196 mg, 0.60 mmol) in N,N-dimethylacetamide (5 mL) was stirred at 120° C. for 16 h. The mixture was cooled, quenched with water (10 mL) and extracted with ethyl acetate (3×10 mL). The organic layers were pooled, washed with brine (10 mL), dried over sodium sulfate, filtered, and concentrated under reduced pressure. The crude product was purified by prep-HPLC (Boston C18 21*250 mm 10 μm column. The mobile phase was acetonitrile/0.01% aqueous ammonium bicarbonate). The product 4-(9-methyl-2-(3-(pyridin-3-yl)-1H-pyrazol-1-yl)-8-(pyridin-4-yl)-9H-purin-6-yl)morpholine (25.6 mg, 0.058 mmol, 19%) was obtained as a white solid. ¹H NMR (400 MHz, Dimethylsulfoxide-d₆) δ 9.17 (d, J=2.3 Hz, 1H), 8.82 (d, J=2.7 Hz, 1H), 8.80-8.76 (m, 2H), 8.59 (dd, J=4.8, 1.6 Hz, 1H), 8.33 (dt, J=7.9, 1.9 Hz, 1H), 7.94-7.87 (m, 2H), 7.51 (dd, J=7.9, 4.8 Hz, 1H), 7.17 (d, J=2.5 Hz, 1H), 4.23 (bs, 4H), 3.97 (s, 3H), 3.80 (t, J=4.8 Hz, 4H); LCMS (ESI) m/z: 440.2 [M+H]⁺.

### Synthesis of 4-(8-(3,6-dihydro-2H-pyran-4-yl)-9-methyl-2-(3-(pyridin-3-yl)-1H-pyrazol-1-yl)-9H-purin-6-yl)morpholine (Compound 95) and 4-(9-methyl-2-(3-(pyridin-3-yl)-1H-pyrazol-1-yl)-8-(tetrahydro-2H-pyran-4-yl)-9H-purin-6-yl)morpholine (Compound 96)

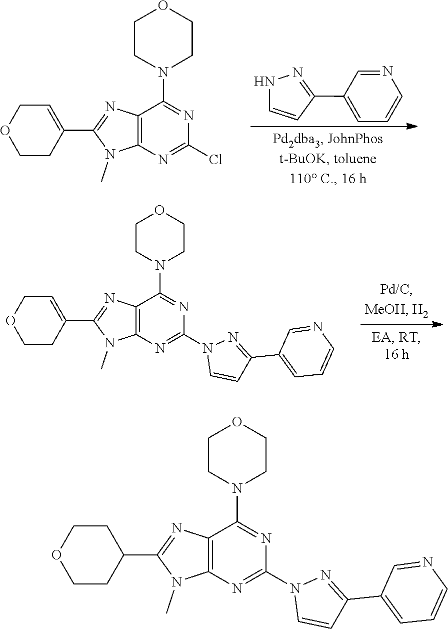

### Step 1: Synthesis of 4-(8-(3,6-Dihydro-2H-pyran-4-yl)-9-methyl-2-(3-(pyridin-3-yl)-1H-pyrazol-1-yl)-9H-purin-6-yl)morpholine

A mixture of 4-(2-chloro-8-(3,6-dihydro-2H-pyran-4-yl)-9-methyl-9H-purin-6-yl)morpholine (200 mg, 0.60 mmol), 3-(1H-pyrazol-3-yl)pyridine (110 mg, 0.76 mmol), tris(dibenzylideneacetone) dipalladium (56 mg, 0.06 mmol), [1,1′-biphenyl]-2-yldi-tert-butylphosphane (36 mg, 0.12 mmol) and potassium tert-butoxide (134 mg, 1.2 mmol) in dry toluene (8 mL) under nitrogen protection was stirred at 110° C. for 16 h. The mixture was cooled to room temperature, quenched with water (10 mL) and extracted with ethyl acetate (10 mL*3). The combined organic phases were washed with water and brine, dried over sodium sulphate, filtered and concentrated. The resultant crude product was purified by prep-HPLC (the crude samples were dissolved in methanol otherwise noted before purified. Boston C18 21*250 mm 10 μm column. The mobile phase was acetonitrile/0.01% aqueous ammonium bicarbonate) to obtain target compound (70 mg, 20.7%) as white solid.

¹H NMR (400 MHz, DMSO-d₆) δ 9.16 (d, J=2.3 Hz, 1H), 8.80 (d, J=2.7 Hz, 1H), 8.58 (dd, J=4.8, 1.7 Hz, 1H), 8.32 (dt, J=8.0, 2.0 Hz, 1H), 7.51 (dd, J=8.0, 4.7 Hz, 1H), 7.16 (d, J=2.7 Hz, 1H), 6.58 (t, J=2.1 Hz, 1H), 4.50-4.30 (m, 6H), 3.90-3.82 (m, 5H), 3.77 (t, J=4.8 Hz, 4H), 2.64-2.58 (m, 2H); LCMS (ESI) m/z: 445.1 [M+H]⁺.

### Step 2: Synthesis of 4-(9-Methyl-2-(3-(pyridin-3-yl)-1H-pyrazol-1-yl)-8-(tetrahydro-2H-pyran-4-yl)-9H-purin-6-yl)morpholine

A mixture of 4-(8-(3,6-dihydro-2H-pyran-4-yl)-9-methyl-2-(3-(pyridin-3-yl)-1H-pyrazol-1-yl)-9H-purin-6-yl)morpholine (30 mg, 0.067 mmol) and Pd/C (10 mg) in methanol (5 mL) and ethyl acetate (2 mL) under hydrogen balloon was stirred at room temperature for 16 h. The mixture was filtered, and the filtrate was concentrated under reduced pressure. The crude product obtained was purified by prep-HPLC (the crude samples were dissolved in methanol otherwise noted before purified. Boston C18 21*250 mm 10 μm column. The mobile phase was acetonitrile/0.01% aqueous ammonium bicarbonate) to obtain the target compound (11.7 mg, 39.2%) as white solid.

¹H NMR (400 MHz, DMSO-d₆) δ 9.16 (d, J=2.3 Hz, 1H), 8.78 (d, J=2.7 Hz, 1H), 8.58 (dd, J=4.8, 1.7 Hz, 1H), 8.32 (dt, J=8.0, 2.0 Hz, 1H), 7.51 (dd, J=7.9, 4.8 Hz, 1H), 7.15 (d, J=2.7 Hz, 1H), 4.30 (bs, 4H), 3.97 (dt, J=11.4, 3.2 Hz, 2H), 3.82-3.72 (m, 7H), 3.51 (td, J=11.2, 3.4 Hz, 2H), 3.29-3.25 (m, 1H), 1.93-1.77 (m, 4H); LCMS (ESI) m/z: 447 [M+H]⁺.

The following compounds were synthesized according to the protocol described above:

| Name | Structure | NMR, MS | # |
|---|---|---|---|
| 4-(9- (cyclopropylmethyl)- 2-(1H-pyrazol-1- yl)-8-(pyridin-4-yl)- 9H-purin-6- yl)morpholine | 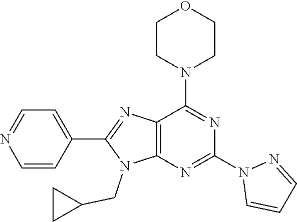 [SMILES: c1cnn(-c2nc(N3CCOCC3)c3nc(-c4ccncc4)n(CC4CC4)c3n2)c1] | ¹H NMR (400 MHz, DMSO-d₆) δ 8.81 (s, 2H), 8.70 (d, J = 2.5 Hz, 1H), 7.89 (d, J = 5.8 Hz, 2H), 7.78 (s, 1H), 6.54 (s, 1H), 4.30 (d, J = 7.0 Hz, 6H), 3.82-3.75 (m, 4H), 1.10 (s, 1H), 0.44-0.37 (m, 2H), 0.34-0.29 (m, 2H); LCMS (ESI) m/z: 403.2 [M + H]⁺. | 97 |
| 4-(9-methyl-2-(1H- pyrazol-1-yl)-8- (pyridin-4-yl)-9H- purin-6- yl)morpholine | 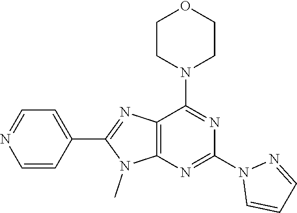 [SMILES: Cn1c(-c2ccncc2)nc2c(N3CCOCC3)nc(-n3cccn3)nc21] | ¹H NMR (400 MHz, Dimethylsulfoxide-d₆) δ 8.78 (dd, J = 4.5, 1.5 Hz, 2H), 8.71 (d, J = 2.2 Hz, 1H), 7.91 (dd, J = 4.5, 1.6 Hz, 2H), 7.78 (d, J = 0.8 Hz, 1H), 6.54 (dd, J = 2.5, 1.6 Hz, 1H), 4.30 (s, 4H), 3.94 (s, 3H), 3.84- 3.71 (m, 4H); LCMS (ESI) m/z: 363.1 [M + H]⁺. | 98 |
| 4-(9-ethyl-2-(1H- pyrazol-1-yl)-8- (pyridin-4-yl)-9H- purin-6- yl)morpholine | 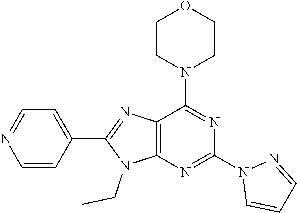 [SMILES: CCn1c(-c2ccncc2)nc2c(N3CCOCC3)nc(-n3cccn3)nc21] | ¹H NMR (400 MHz, DMSO-d₆) δ 8.98 (d, J = 4.8 Hz, 2H), 8.72 (d, J = 2.4 Hz, 1H), 8.28 (d, J = 6.2 Hz, 2H), 7.80 (s, 1H), 6.67-6.47 (m, 1H), 4.75 (bs, 4H), 4.50 (q, J =7.2 Hz, 2H), 3.86-3.73 (m, 4H), 1.38 (t, J = 7.2 Hz, 3H); LCMS (ESI) m/z: 377.0 [M + H]⁺. | 99 |
| 4-(2-(4- cyclopropyl-1H- pyrazol-1-yl)-9- methyl-8-(pyridin- 4-yl)-9H-purin-6- yl)morpholine | 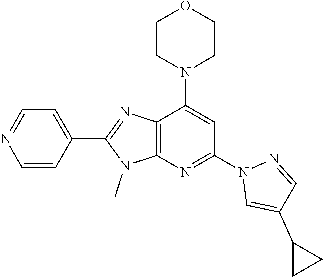 [SMILES: Cn1c(-c2ccncc2)nc2c(N3CCOCC3)cc(-n3cc(C4CC4)cn3)nc21] | H NMR (400 MHz, DMSO-d₆) δ 8.78 (d, J = 5.7 Hz, 2H), 8.43 (s, 1H), 7.90 (d, J = 5.6 Hz, 2H), 7.59 (s, 1H), 4.32 (s, 4H), 3.92 (s, 3H), 3.80-3.76 (m, 4H), 1.81 (sept, J = 1.6 Hz, 1H), 0.88 (dd, J = 12H, 8.0 Hz, 2H), 0.63 (dd, J = 12, 4.0 Hz, 2H); LCMS: (ESI) m/z: 403.0 [M + H]⁺. | 100 |
| 4-(9-ethyl-2-(1H- pyrazol-1-yl)-8- (pyridin-4-yl)-9H- purin-6-yl)-3- methylmorpholine | 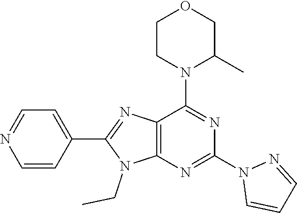 [SMILES: CCn1c(-c2ccncc2)nc2c(N3CCOCC3C)nc(-n3cccn3)nc21] | 1H NMR (400 MHz, DMSO-d₆) δ 8.90 (bs, 2H), 8.70(s, 1H), 7.90 (s, 2H), 7.79 (s, 1H), 6.55 (s, 1H), 4.42 (d, J = 6.4 Hz, 2H), 4.02 (d, J = 8.1 Hz, 1H), 3.81 (d, J = 11.5 Hz, 1H), 3.72 (dd, J = 11.6, 2.7Hz, 1H), 3.57 (t, J = 10.8 Hz, 2H), 3.30 (s, 2H), 1.40-1.20 (m, 6H). LCMS (ESI) m/z: 391.1 [M + H]⁺ | 101 |
| 4-(2-(4-chloro-1H- pyrazol-1-yl)-9- methyl-8-(pyridin- 4-yl)-9H-purin-6- yl)morpholine | 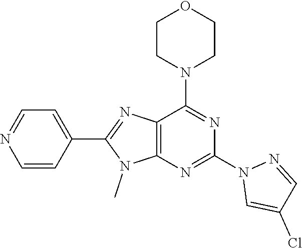 [SMILES: Cn1c(-c2ccncc2)nc2c(N3CCOCC3)nc(-n3cc(Cl)cn3)nc21] | ¹H NMR (400 MHz, DMSO-d₆) δ 8.95 (s, 1H), 8.79 (d, J = 6.1 Hz, 2H), 7.91 (dd, J = 4.4, 1.6 Hz, 3H), 4.12 (bs, 4H), 3.94 (s, 3H), 3.78 (d, J = 4.5 Hz, 4H)I LCMS (ESI) m/z: 396.9. [M + H]+. | 102 |
| 4-(9-ethyl-8-(1- methyl-1H- pyrazol-5-yl)-2- (1H-pyrazol-1-yl)- 9H-purin-6- yl)morpholine | 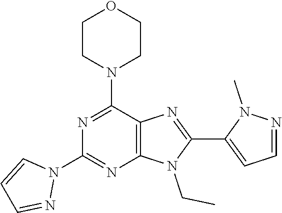 [SMILES: CCn1c(-c2ccnn2C)nc2c(N3CCOCC3)nc(-n3cccn3)nc21] | ¹H NMR (400 MHz, DMSO-d₆) δ 8.69 (d, J = 2.4 Hz, 1H), 7.78 (s, 1H), 7.67 (d, J = 1.9 Hz, 1H), 6.88 (d, J = 1.9 Hz, 1H), 6.60-6.48 (m, 1H), 4.50-4.35 (m, 4H), 4.29 (d, J = 7.2 Hz, 2H), 4.05 (s, 3H), 3.83-3.69 (m, 4H), 1.33 (t, J = 7.2 Hz, 3H); LCMS (ESI) m/z: 379.8[M + H]+. | 103 |
| 4-(9-ethyl-2-(4- methoxy-1H- pyrazol-1-yl)-8- (pyridin-4-yl)-9H- purin-6- yl)morpholine | 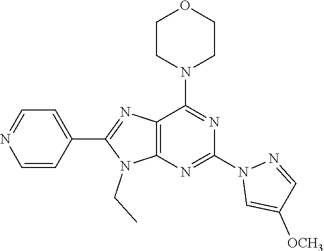 [SMILES: CCn1c(-c2ccncc2)nc2c(N3CCOCC3)nc(-n3cc(OC)cn3)nc21] | 1H NMR (400 MHz, DMSO-d₆) δ 8.79 (d, J = 6.0 Hz, 2H), 8.33 (d, J = 0.6 Hz, 1H), 7.84 (dd, J = 4.6, 1.5 Hz, 2H), 7.60 (d, J = 0.6 Hz, 1H), 4.43 (s, 2H), 4.41 (q, J = 7.2 Hz, 2H), 4.36- 4.08 (m, 2H), 3.81 (s, 3H), 3.80-3.75 (m, 4H), 1.32 (t, J = 7.2 Hz, 3H); LCMS (ESI) m/z: 407.0. [M + H]+. | 104 |
| 5-(9-ethyl-2-(1H- pyrazol-1-yl)-8- (pyridin-4-yl)-9H- purin-6-yl)-2-oxa- 5- azabicyclo[2.2.1] heptane | 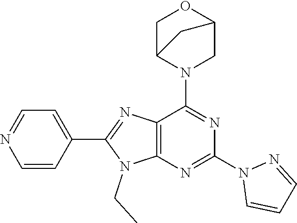 [SMILES: CCn1c(-c2ccncc2)nc2c(N3CC4CC3CO4)nc(-n3cccn3)nc21] | 1H NMR (400 MHz, DMSO-d₆) δ 8.80 (d, J = 5.6 Hz, 2H), 8.76-8.60 (m, 1H), 7.88-7.84 (m, 2H), 7.78 (d, J = 0.8 Hz, 1H), 6.54 (dd, J = 2.5, 1.6 Hz, 1H), 6.11 (s, 1H), 4.78 (s, 1H), 4.42 (q, J = 7.3 Hz, 2H), 4.11-3.65 (m, 4H), 2.09-1.81 (m, 2H), 1.34 (t, J = 7.2 Hz, 3H); LCMS (ESI) m/z: 389.0 [M + H]+. | 105 |
| 4-(9-ethyl-2-(3- methyl-4-phenyl- 1H-pyrazol-1-yl)-8- (pyridin-4-yl)-9H- purin-6- yl)morpholine | 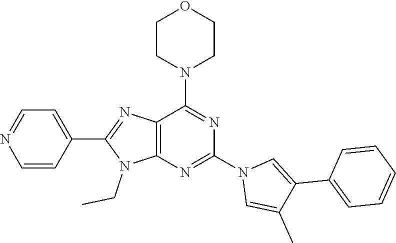 [SMILES: CCn1c(-c2ccncc2)nc2c(N3CCOCC3)nc(-n3cc(C)c(-c4ccccc4)c3)nc21] | 1H NMR (400 MHz, DMSO-d₆) δ 8.89-8.76 (m, 3H), 7.85 (d, J = 5.9 Hz, 2H), 7.60 (d, J = 7.2 Hz, 2H), 7.45 (t, J = 7.6 Hz, 2H), 7.33 (t, J = 7.3 Hz, 1H), 4.46 (s, 2H), 4.44 (d, J = 7.2 Hz, 2H), 4.37-4.01 (m, 2H), 3.79 (s, 4H), 2.45 (s, 3H), 1.34 (t, J = 7.1 Hz, 3H); LCMS (ESI) m/z: 467.0. [M + H]+. | 106 |
| 4-(9-ethyl-2-(4- phenyl-1H- pyrazol-1-yl)-8- (pyridin-4-yl)-9H- purin-6-yl)-3- methylmorpholine | 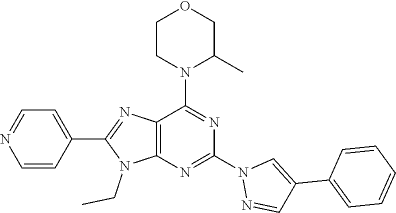 [SMILES: CCn1c(-c2ccncc2)nc2c(N3CCOCC3C)nc(-n3cc(-c4ccccc4)cn3)nc21] | 1H NMR (400 MHz, DMSO-d₆) δ 9.10 (s, 1H), 8.81 (d, J = 5.8 Hz, 2H), 8.28 (s, 1H), 7.87 (d, J = 6.0 Hz, 2H), 7.81 (d, J = 7.2 Hz, 2H), 7.43 (s, 2H), 7.28 (t, J = 7.4 Hz, 1H), 6.07-5.36 (m, 1H), 5.37-4.81 (m, 1H), 4.45 (q, J = 7.3 Hz, 2H), 4.04 (d, J = 8.3 Hz, 1H), 3.82 (s, 1H), 3.75 (d, J = 11.1 Hz, 1H), 3.57 (s, 2H), 1.41- 1.33 (m, 6H); LCMS (ESI) m/z: 467.1 [M + H]+ | 107 |
| 4-(9-methyl-2-(4- phenyl-1H- pyrazol-1-yl)-8- (pyridin-4-yl)-9H- purin-6- yl)morpholine | 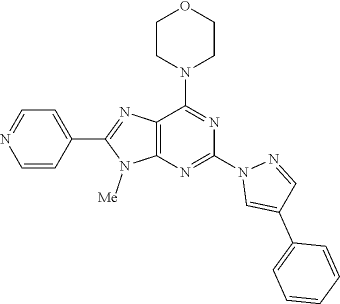 [SMILES: Cn1c(-c2ccncc2)nc2c(N3CCOCC3)nc(-n3cc(-c4ccccc4)cn3)nc21] | ¹H NMR (400 MHz, DMSO-d₆) δ 9.13 (d, J = 0.7 Hz, 1H), 8.79 (dd, J = 4.5,1.6 Hz, 2H), 8.27 (d, J = 0.7 Hz, 1H), 7.93 (dd, J = 4.5,1.6 Hz, 2H), 7.86-7.73 (m, 2H), 7.42 (t, J = 7.7 Hz, 2H), 7.28 (t, J = 7.4 Hz, 1H), 4.44-4.39 (m, 4H), 3.97 (s, 3H), 3.86-3.69 (m, 4H); LCMS (ESI) m/z: 439.2 [M + H]⁺. | 108 |
| 4-(9-methyl-8- (pyridin-4-yl)-2-(4- (pyridin-4-yl)-1H- pyrazol-1-yl)-9H- purin-6- yl)morpholine | 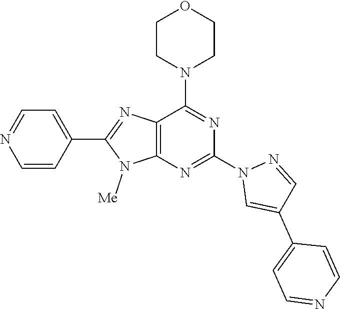 [SMILES: Cn1c(-c2ccncc2)nc2c(N3CCOCC3)nc(-n3cc(-c4ccncc4)cn3)nc21] | ¹H NMR (400 MHz, DMSO-d₆) δ 9.36 (s, 1H), 8.80 (d, J = 5.6 Hz, 2H), 8.58 (s, 2H), 8.44 (s, 1H), 7.93 (dd, J = 4.5,1.6 Hz, 2H), 7.84 (d, J = 5.8 Hz, 2H), 4.44 (s, 4H), 3.98 (s, 3H), 3.81 (s, 4H); LCMS (ESI) m/z: 440.1 [M + H]⁺. | 109 |
| 4-(9-ethyl-2-(4- methyl-3-phenyl- 1H-pyrazol-1-yl)-8- (pyridin-4-yl)-9H- purin-6- yl)morpholine | 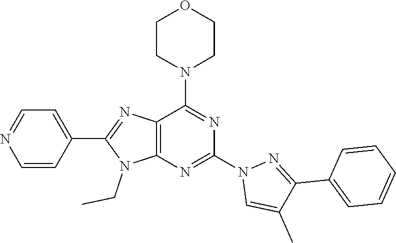 | ¹H NMR (500 MHz, DMSO-d₆) δ 8.79 (dd, J = 4.5, 1.6 Hz, 2H), 8.59 (d, J = 0.9 Hz, 1H), 7.84 (dd, J = 4.5, 1.6 Hz, 2H), 7.81-7.74 (m, 2H), 7.50 (t, J = 7.6 Hz, 2H), 7.42 (t, J = 7.4 Hz, 1H), 4.64-4.01 (m, 6H), 3.83-3.75 (m, 4H), 2.29 (s, 3H), 1.34 (t, J = 7.2 Hz, 3H); LCMS (ESI) m/z: 466.8[M + H]+. | 110 |
| 4-(9-cyclopropyl-2- (3-cyclopropyl-1H- pyrazol-1-yl)-8- (pyridin-4-yl)-9H- purin-6- yl)morpholine | 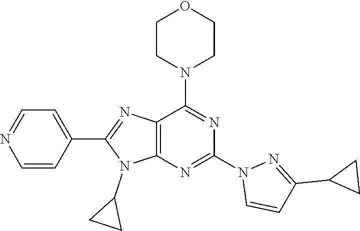 [SMILES: c1cc(-c2nc3c(N4CCOCC4)nc(-n4ccc(C5CC5)n4)nc3n2C2CC2)ccn1] | ¹H NMR (400 MHz, DMSO-d₆) δ 8.77 (d, J = 5.7 Hz, 2H), 8.52 (d, J = 2.4 Hz, 1H), 7.99 (d, J = 5.8 Hz, 2H), 6.21 (d, J = 2.4 Hz, 1H), 4.31 (bs, 4H), 3.77 (s, 5H), 2.04 (pent, J = 4Hz, 1H), 1.14 (d, J = 6.2 Hz, 2H), 1.05- 0.90 (m, 2H), 0.81 (s, 2H), 0.74 (d, J = 4 Hz, 2H); LCMS (ESI) m/z: 428.9[M + H]+. | 111 |
| (R)-4-(9-ethyl-2- (4-phenyl-1H- pyrazol-1-yl)-8- (pyridin-4-yl)-9H- purin-6-yl)-3- methylmorpholine | 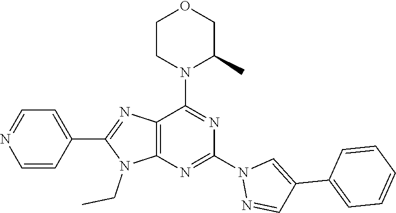 [SMILES: CCn1c(-c2ccncc2)nc2c(N3CCOC[C@H]3C)nc(-n3cc(-c4ccccc4)cn3)nc21] | 1H NMR (400 MHz, DMSO-d₆) δ 9.09 (s, 1H), 8.81 (d, J = 5.1 Hz, 2H), 8.27 (s, 1H), 7.86 (d, J = 5.7 Hz, 2H), 7.80 (d, J = 7.4 Hz, 2H), 7.43 (t, J = 7.7 Hz, 2H), 7.28 (t, J = 7.4 Hz, 1H), 5.49 (bs, 1H), 4.44 (q, J = 7.3 Hz, 2H), 4.04 (d, J = 8.5 Hz, 1H), 3.83 (d, J = 12H, 1H), 3.75 (d, J = 10.4 Hz, 1H), 3.59 (t, J = 10.4 Hz, 2H), 1.42-1.32 (m, 6H); LCMS (ESI) m/z: 467.3 [M + H]⁺ | 113 |
| (S)-4-(9-ethyl-2-(4- phenyl-1H- pyrazol-1-yl)-8- (pyridin-4-yl)-9H- purin-6-yl)-3- methylmorpholine | 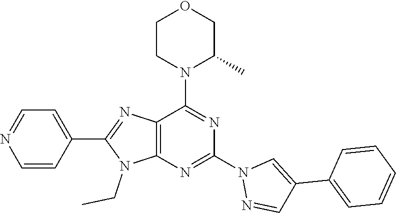 [SMILES: CCn1c(-c2ccncc2)nc2c(N3CCOC[C@@H]3C)nc(-n3cc(-c4ccccc4)cn3)nc21] | 1H NMR (400 MHz, DMSO-d₆) δ 9.09 (s, 1H), 8.80 (dd, J = 4.5, 1.6 Hz, 2H), 8.27 (d, J = 0.8 Hz, 1H), 7.86 (dd, J = 4.5, 1.6 Hz, 2H), 7.82- 7.77 (m, 2H), 7.43 (t, J = 7.7 Hz, 2H), 7.28 (t, J = 7.4 Hz, 1H), 5.53 (bs, 2H), 4.44 (q, J = 7.2 Hz, 2H), 4.04 (d, J = 8.2 Hz, 1H), 3.83 (d, J = 11.5 Hz, 1H), 3.75 (dd, J = 11.7, 2.9 Hz, 1H), 3.60 (t, J = 10.4 Hz, 2H), 1.42-1.33 (m, 6H); LCMS: (ESI) m/z 466.8 [M + H]⁺. | 114 |

### Synthesis of 4-(9-cyclopropyl-2-(5-cyclopropyl-1H-pyrazol-1-yl)-8-(pyridin-4-yl)-9H-purin-6-yl)morpholine (Compound 115)

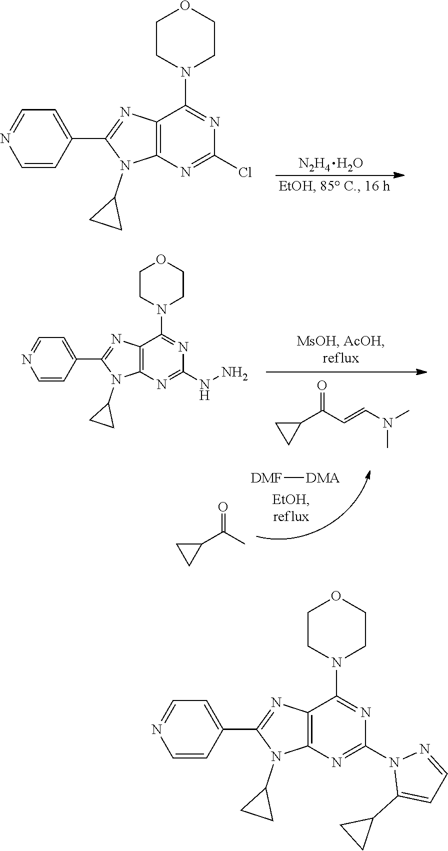

### Step 1a: Preparation of (E)-1-cyclopropyl-3-(dimethylamino)prop-2-en-1-one

The solution of 1-cyclopropylethan-1-one (840 mg, 10 mmol) in N,N-dimethylformamide dimethyl acetal (15 mL) was stirred at 110° C. for 16 h. The reaction mixture was concentrated to give the desired product (400 mg, 28%) as a yellow oil. It was directly used in the step-2.

### Step 1: Synthesis of 4-(9-cyclopropyl-2-hydrazineyl-8-(pyridin-4-yl)-9H-purin-6-yl)morpholine

A mixture of 4-(2-chloro-9-cyclopropyl-8-(pyridin-4-yl)-9H-purin-6-yl)morpholine (200 mg, 0.561 mmol) and hydrazine hydrate (5 mL) in ethanol (20 mL) was stirred at 85° C. for 16 h. The reaction mixture was concentrated and purified by silica gel column chromatography (petroleum ether:ethyl acetate=5:1) to give the desired product (180 mg, 91%) as a yellow solid.

### Step 2: Preparation of 4-(9-cyclopropyl-2-(5-cyclopropyl-1H-pyrazol-1-yl)-8-(pyridin-4-yl)-9H-purin-6-yl)morpholine

The mixture of 4-(9-cyclopropyl-2-hydrazineyl-8-(pyridin-4-yl)-9H-purin-6-yl)morpholine (45 mg, 0.128 mmol) and (E)-1-cyclopropyl-3-(dimethylamino)prop-2-en-1-one (400 mg, crude) in methanol (5 mL) and acetic acid (5 mL) was stirred at 85° C. for 16 h. The reaction mixture was filtered and purified by prep-HPLC (SunFire C18, 4.6*50 mm, 3.5 um column Xbridge C18 3.5 μm 4.6×50 mm column. The mobile phase was acetonitrile/10 mM ammonium bicarbonate aqueous solution) to give the desired product as off-white solid (17.2 mg, 31%).

¹H NMR (400 MHz, CDCl₃) δ 8.79 (dd, J=4.6, 1.4 Hz, 2H), 7.89 (dd, J=4.6, 1.5 Hz, 2H), 7.62 (d, J=1.6 Hz, 1H), 6.02 (d, J=1.1 Hz, 1H), 4.41 (bs, 4H), 3.93-3.75 (m, 4H), 3.59-3.49 (m, 1H), 2.79-2.65 (m, 1H), 1.22 (q, J=6.9 Hz, 2H), 1.04-0.85 (m, 4H), 0.80-0.68 (m, 2H). LCMS (ESI) m/z: 429.1 [M+H]⁺.

### Synthesis of 1-methyl-5-(1-(9-methyl-6-morpholino-8-(pyridin-4-yl)-9H-purin-2-yl)-1H-pyrazol-3-yl)piperidin-2-one (Compound 116)

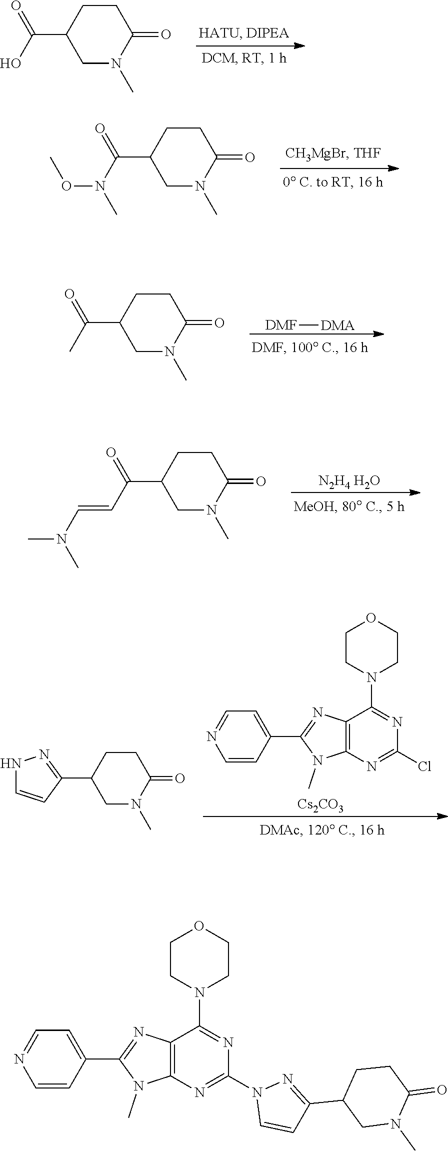

### Step 1: Preparation of N-methoxy-N,1-dimethyl-6-oxopiperidine-3-carboxamide

A mixture of 1-methyl-6-oxopiperidine-3-carboxylic acid (300 mg, 1.91 mmol), DIPEA (1.26 mL, 7.6 4 mmol) and HATU (1.1 g, 2.86 mmol) in THE (10 mL) was stirred at room temperature for 30 min, then N, O-dimethylhydroxylamine hydrochloride (279 mg, 2.86 mmol) was added and the resultant mixture was stir red at room temperature for 1 h. The mixture was concentrated and purified by column (5% MeOH in DCM) to give N-methoxy-N,1-dimethyl-6-oxopiperidine-3-carboxamide as white solid (350 mg, 92%). LCMS (E SI) m/z: 201 [M+H]⁺.

### Step 2: Preparation of 5-acetyl-1-methylpiperidin-2-one

To a solution of N-methoxy-N,1-dimethyl-6-oxopiperidine-3-carboxamide (300 mg, 1.5 mmol) in THF (8 mL) was added methylmagnesium bromide (0.65 mL, 1.95 mmol) slowly at 0° C. under nitrogen atmosphere and the mixture was warmed up and stirred at room temperature for 16 h. Saturated NH₄Cl (3 mL) solution was added into the mixture and concentrated. The crude product was purified by column chromatography (5% MeOH in DCM) to obtain 5-acetyl-1-methylpiperidin-2-one as colorless oil (150 mg, 65%). L CMS (ESI) m/z: 156 [M+H]⁺.

### Step 3: Preparation of (E)-5-(3-(dimethylamino)acryloyl)-1-methylpiperidin-2-one

A mixture of 5-acetyl-1-methylpiperidin-2-one (80 mg, 0.52 mmol) in DMF-DMA (5 mL) was stirred at 110° C. for 16 h and then concentrated. The crude product thus obtained was purified by column chromatography (8% MeOH in DCM) to obtain (E)-5-(3-(dimethylamino)acryloyl)-1-methylpiperidin-2-one as white solid (80 mg, 65%). LCMS (ESI) m/z: 311 [M+H]⁺.

### Step 4: Preparation of 1-methyl-5-(1H-pyrazol-3-yl)piperidin-2-one

A mixture of (E)-5-(3-(dimethylamino)acryloyl)-1-methylpiperidin-2-one (80 mg, 0.38 mmol) and NH₂NH₂OH (5 mL) in EtOH (5 mL) was stirred at 80° C. for 6 h under nitrogen atmosphere. The mixture was concentrated to give 1-methyl-5-(1H-pyrazol-3-yl)piperidin-2-one as white solid (60 mg, 88%). LCMS (ESI) m/z: 180 [M+H]⁺.

### Step 5: Preparation of 1-methyl-5-(1-(9-methyl-6-morpholino-8-(pyridin-4-yl)-9H-purin-2-yl)-1H-pyrazol-3-yl)piperidin-2-one

A mixture of 1-methyl-5-(1H-pyrazol-3-yl)piperidin-2-one (50 mg, 0.15 mmol), 4-(2-chloro-9-methyl-8-(pyridin-4-yl)-9H-purin-6-yl)morpholine (33 mg, 0.18 mmol) and Cs₂CO₃ (148 mg, 0.45 mmol) in DMAc (5 mL) was stirred at 120° C. for 16 h. The resultant mixture purified by prep-HPLC to give 1-methyl-5-(1-(9-m ethyl-6-morpholino-8-(pyridin-4-yl)-9H-purin-2-yl)-1H-pyrazol-3-yl)piperidin-2-one as white solid (21.7 mg, 40%).

¹H NMR (400 MHz, DMSO-d₆) δ 8.79-8.78 (m, 2H), 8.65 (d, J=2.0 Hz, 1H), 7.91-7.90 (m, 2H), 6.50 (d, J=2.0 Hz, 1H), 4.41-4.24 (m, 4H), 3.93 (s, 3H), 3.79-3.77 (m, 4H), 3.60-3.47 (m, 2H), 3.29-3.25 (m, 1H), 2.87 (s, 3H), 2.45-2.29 (m, 2H), 2.12-1.88 (m, 2H); LCMS (ESI) m/z: 474.3 [M+H]⁺.

### Synthesis of 4-(9-cyclopropyl-2-(1H-pyrazol-1-yl)-8-(pyridin-4-yl)-9H-purin-6-yl)morpholine (Compound 117)

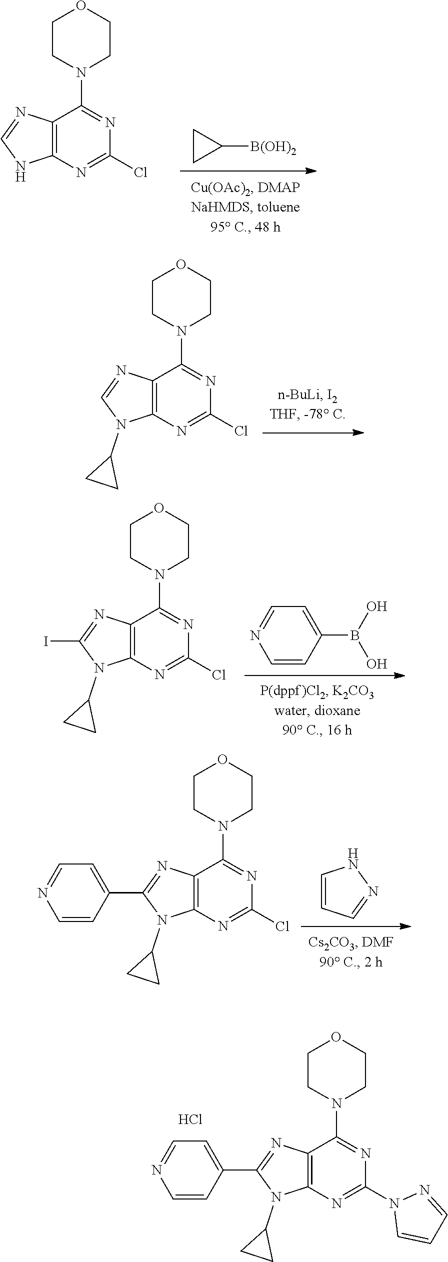

### Step 1: Preparation of 4-(2-chloro-9-cyclopropyl-9H-purin-6-yl)morpholine

To a solution of 4-(2-chloro-9H-purin-6-yl)morpholine (1.0 mg, 4.18 mmol) in toluene (15 mL) were added cyclopropylboronic acid (718.1 mg, 8.37 mmol), cupric acetate (784.1 mg, 4.18 mmol), 4-dimethylaminepyridine (1.53 g, 12.54 mmol) and sodium bis(trimethylsilyl)amide (4.18 mL) at 25° C. and the resultant mixture was stirred at 95° C. for 48 h under nitrogen protection. The mixture was then extracted with ethyl acetate (20 mL*2) and washed with water (10 mL*2). The organic layer was dried over sodium sulfate and concentrated and the residue was purified by silica gel column chromatography (45% ethyl acetate in petroleum ether) to obtain 4-(2-chloro-9-cyclopropyl-9H-purin-6-yl)morpholine as white solid (600.0 mg, 51.5%). LCMS (ESI) m/z: 280.0 [M+H]⁺.

### Step 2: Preparation of 4-(2-chloro-9-cyclopropyl-8-iodo-9H-purin-6-yl)morpholine

A mixture of 4-(2-chloro-9-cyclopropyl-9H-purin-6-yl)morpholine (500 mg, 1.79 mmol) and n-butyllithium (1.0 mL, 2.33 mmol) in tetrahydrofuran (10 mL) was stirred at −78° C. for 1 h. Then Iodine (1.25 g, 5.37 mmol) was added and the mixture was warmed up and stirred at 25° C. for 2 h. The reaction was quenched with water and extracted with ethyl acetate (20 mL*2). The combined organic layer was washed with water (10 mL*2), dried over sodium sulfate and concentrated. The residue was purified by silica gel column chromatography (21% ethyl acetate in petroleum ether) to obtain 4-(2-chloro-9-cyclopropyl-8-iodo-9H-purin-6-yl)morpholine as white solid (360.0 mg, 49.7%). LCMS (ESI) m/z: 405.8 [M+H]⁺.

### Step 3: Preparation of 4-(2-chloro-9-cyclopropyl-8-(pyridin-4-yl)-9H-purin-6-yl)morpholine

To a solution of 4-(2-chloro-9-cyclopropyl-8-iodo-9H-purin-6-yl)morpholine (360 mg, 0.9 mmol) in dioxane (6 mL) and water (1 mL) were added pyridin-4-ylboronic acid (71.9 mg, 123.05 mmol), [1,1′-bis(diphenylphosphino)ferrocene]dichloropalladium(II) (47.5 mg, 0.09 mmol) and potassium carbonate (269.5 mg, 2.7 mmol) at 25° C. and the resultant mixture was stirred at 90° C. for 16 h under nitrogen. The mixture was then extracted with dichloromethane (20 mL*2) and washed with water (10 mL*2). The organic layer was dried over sodium sulfate, concentrated and the residue was purified by silica gel column chromatography (3% methanol in dichloromethane) to obtain 4-(2-chloro-9-cyclopropyl-8-(pyridin-4-yl)-9H-purin-6-yl)morpholine as white solid (250 mg, 78.1%). LCMS (ESI) m/z: 357.0 [M+H]⁺.

### Step 4: Preparation of 4-(9-cyclopropyl-2-(1H-pyrazol-1-yl)-8-(pyridin-4-yl)-9H-purin-6-yl)morpholine hydrochloride

To a solution of 4-(2-chloro-9-cyclopropyl-8-(pyridin-4-yl)-9H-purin-6-yl)morpholine (100.0 mg, 0.28 mmol) in N,N-dimethylformamide (2 mL) were added 1H-pyrazole (28.5 mg, 0.42 mmol) and cesium carbonate (273.0 mg, 0.84 mmol) and the resultant mixture was stirred at 90° C. for 2 h. It was then extracted with dichloromethane (20 mL*2) and washed with water (10 mL*2). The organic layer was dried over sodium sulfate and concentrated. The crude product was purified by pre-HPLC (the crude samples were dissolved in N,N-dimethylformamide otherwise noted before purified. BOSTON pHlex ODS 10 um 21.2×250 mm 120 A. The mobile phase was acetonitrile/0.1% Ammonium bicarbonate to give the product as white solid. The white solid was added hydrochloric acid (3M, 0.5 mL) and re-crystallized from water, dried by lyophilization to give the product 4-(9-cyclopropyl-2-(1H-pyrazol-1-yl)-8-(pyridin-4-yl)-9H-purin-6-yl)morpholine hydrochloride as yellow solid (44.4 mg, 40.8%).

¹H NMR (400 MHz, DMSO-d₆) δ 8.90 (s, 2H), 8.69 (d, J=2.2 Hz, 1H), 8.27 (d, J=4.0 Hz, 2H), 7.80 (s, 1H), 6.56 (s, 1H), 4.34-3.93 (m, 4H), 3.83-3.76 (m, 5H), 1.18 (d, J=6.1 Hz, 2H), 0.86 (d, J=8.7 Hz, 2H); LCMS (ESI) m/z: 389.0 [M+H]⁺.

### Synthesis of Preparation of 4-(9-ethyl-2-(4-methyl-1H-pyrazol-1-yl)-8-(pyridin-4-yl)-9H-purin-6-yl)morpholine (Compound 118)

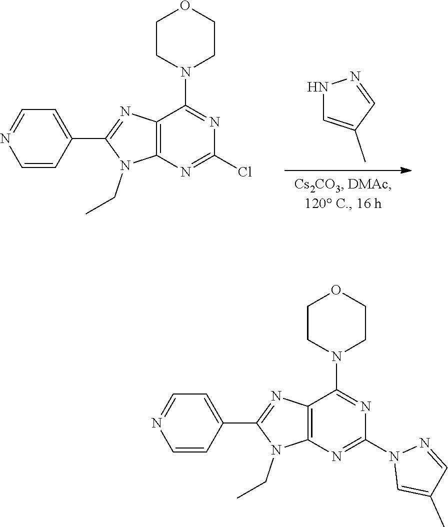

A mixture of 4-(2-chloro-9-ethyl-8-(pyridin-4-yl)-9H-purin-6-yl)morpholine (100 mg, 0.29 mmol), 4-methyl-1H-pyrazole (30 mg, 0.37 mmol) and cesium carbonate (293 mg, 0.87 mmol) in N,N-dimethylacetamide (3 mL) was stirred at 120° C. under nitrogen atmosphere for 16 h. The mixture was filtered and purified by prep-HPLC (Column Xbridge 21.2*250 mm C18, 10 um, Mobile Phase A: water (10 mmol/L ammonium bicarbonate) B: acetonitrile) to afford 4-(9-ethyl-2-(4-methyl-1H-pyrazol-1-yl)-8-(pyridin-4-yl)-9H-purin-6-yl)morpholine as a white solid. (69.1 mg, 61.03%)

1H NMR (400 MHz, DMSO-d₆) b 8.79 (dd, J=4.5, 1.5 Hz, 2H), 8.47 (s, 1H), 7.84 (dd, J=4.5, 1.6 Hz, 2H), 7.60 (s, 1H), 4.40 (q, J=7.2 Hz, 6H), 3.81-3.73 (m, 4H), 2.11 (s, 3H), 1.33 (t, J=7.2 Hz, 3H); LCMS (ESI) m/z: 391.0. [M+H]+.

The following compounds were synthesized according to the protocol described above:

| Name | Structure | NMR, MS | # |
|---|---|---|---|
| 4-(9-cyclopropyl-2- (4-methoxy- pyrazol-1-yl)-8- (pyridin-4-yl)-9H- purin-6- yl)morpholine | 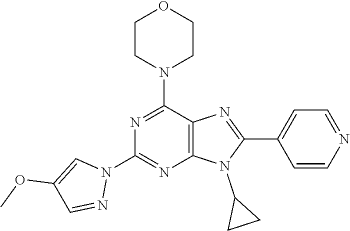 [SMILES: COc1cnn(-c2nc(N3CCOCC3)c3nc(-c4ccncc4)n(C4CC4)c3n2)c1] | 1H NMR (400 MHz, DMSO-d6) δ 8.77 (dd, J = 4.5, 1.6 Hz, 2H), 8.29 (d, J = 0.8 Hz, 1H), 7.99 (dd, J = 4.5, 1.6 Hz, 2H), 7.61 (d, J = 0.8 Hz, 1H), 4.32 (s, 4H), 3.81 (s, 3H), 3.80- 3.73 (m, 5H), 1.14 (q, J = 7.0 Hz, 2H), 0.85- 0.79 (m, 2H). LCMS (ESI) m/z: 419.1. [M + H]+. | 112 |
| 4-(9- (difluoromethyl)-2- (1H-pyrazol-1-yl)- 8-(pyridin-4-yl)-9H- purin-6- yl)morpholine | 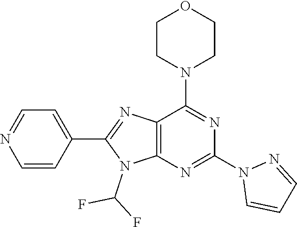 [SMILES: FC(F)n1c(-c2ccncc2)nc2c(N3CCOCC3)nc(-n3cccn3)nc21] | 1H NMR (400 MHz, DMSO-d₆) δ 8.82 (dd, J = 4.6, 1.4 Hz, 2H), 8.74 (d, J = 2.5 Hz, 1H), 8.13 (t, J = 57.5 Hz, 1H), 7.82 (dd, J = 4.6, 1.2 Hz, 3H), 6.58 (dd, J = 2.3, 1.7 Hz, 1H), 4.32 (d, J = 181.8 Hz, 4H), 3.85-3.73 (m, 4H); LCMSA011, [M + H]+ = 398.9. | 119 |
| 3-methyl-4-(9- methyl-2-(1H- pyrazol-1-yl)-8- (pyridin-4-yl)-9H- purin-6- yl)morpholine | 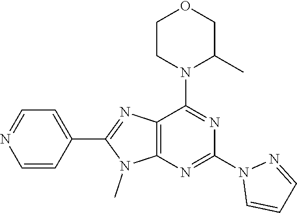 [SMILES: CC1COCCN1c1nc(-n2cccn2)nc2c1nc(-c1ccncc1)n2C] | 1H NMR (400 MHz, DMSO-d₆) δ 8.88-8.63 (m, 3H), 7.85 (d, J = 54.2 Hz, 3H), 6.62-6.50 (m, 1H), 5.50 (s, 2H), 4.02 (d, J = 7.9 Hz, 1H), 3.94 (s, 3H), 3.82 (d, J = 11.5 Hz, 1H), 3.77- 3.70 (m, 1H), 3.64-3.47 (m, 2H), 1.38 (d, J = 6.7 Hz, 3H); LCMS (ESI) m/z: 376.8 [M + H]+ | 120 |
| 4-(9-(2- methoxyethyl)-2- (1H-pyrazol-1-yl)- 8-(pyridin-4-yl)-9H- purin-6- yl)morpholine | 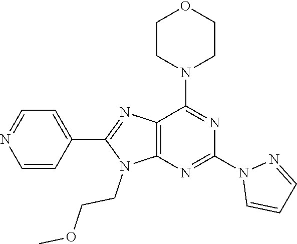 [SMILES: COCCn1c(-c2ccncc2)nc2c(N3CCOCC3)nc(-n3cccn3)nc21] | ¹H NMR (400 MHz, DMSO-d₆) δ 8.79 (s, 2H), 8.70 (d, J = 2.3 Hz, 1H), 7.90 (d, J = 5.9 Hz, 2H), 7.78 (s, 1H), 6.59-6.50 (m, 1H), 4.54 (t, J = 5.2 Hz, 2H), 4.49-3.74 (m, 8H), 3.71 (t, J = 5.3 Hz, 2H), 3.10 (s, 3H); LCMS (ESI) m/z: 407.1 [M + H]⁺. | 121 |
| 4-(9-propyl-2-(1H- pyrazol-1-yl)-8- (pyridin-4-yl)-9H- purin-6- yl)morpholine | 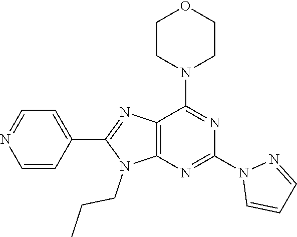 [SMILES: CCCn1c(-c2ccncc2)nc2c(N3CCOCC3)nc(-n3cccn3)nc21] | ¹H NMR (400 MHz, DMSO-d₆) δ 8.79 (d, J = 5.6 Hz, 2H), 8.69 (d, J = 2.1 Hz, 1H), 7.84 (dd, J = 4.5, 1.5 Hz, 2H), 7.78 (d, J = 0.8 Hz, 1H), 6.54 (dd, J = 2.5, 1.6 Hz, 1H), 4.65- 3.90 (m, 6H), 3.85-3.73 (m, 4H), 1.71 (dd, J = 14.7, 7.4 Hz, 2H), 0.77 (t, J = 7.4 Hz, 3H); LCMS (ESI) m/z: 391.0 [M + H]⁺. | 122 |
| 4-(9-ethyl-2-(4- phenyl-1H-pyrazol- 1-yl)-8-(pyridin-4- yl)-9H-purin-6- yl)morpholine | 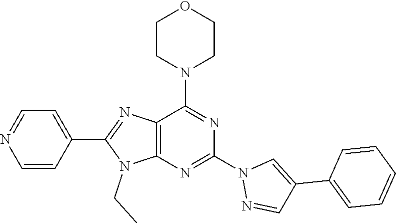 [SMILES: CCn1c(-c2ccncc2)nc2c(N3CCOCC3)nc(-n3cc(-c4ccccc4)cn3)nc21] | ¹H NMR (400 MHz, DMSO-d₆) δ 9.11 (s, 1H), 8.80 (d, J = 6.1 Hz, 2H), 8.27 (s, 1H), 7.85 (dd, J = 4.5, 1.6 Hz, 2H), 7.81 (d, J = 7.2 Hz, 2H), 7.42 (t, J = 7.7 Hz, 2H), 7.28 (t, J = 7.4 Hz, 1H), 4.45 (dd, J = 14.5, 7.2 Hz, 6H), 3.83- 3.78 (m, 4H), 1.35 (t, J = 7.2 Hz, 3H); LCMS (ESI) m/z: 453.0 [M + H]⁺. | 123 |
| 4-(9-cyclopropyl-2- (4-cyclopropyl-1H- pyrazol-1-yl)-8- (pyridin-4-yl)-9H- purin-6- yl)morpholine | 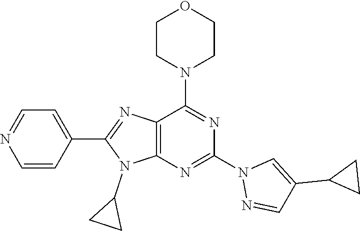 [SMILES: c1cc(-c2nc3c(N4CCOCC4)nc(-n4cc(C5CC5)cn4)nc3n2C2CC2)ccn1] | ¹H NMR (500 MHz, DMSO-d₆) δ 8.76 (d, J = 5.2 Hz, 2H), 8.39 (s, 1H), 7.98 (d, J = 5.9 Hz, 2H), 7.58 (s, 1H), 4.33 (bs, 4H), 3.79- 3.72 (m, 5H), 1.86-1.76 (m, 1H), 1.14 (q, J = 6.7 Hz, 2H), 0.92-0.86 (m, 2H), 0.85- 0.79 (m, 2H), 0.66-0.58 (m, 2H); LCMS (ESI) m/z: 429.1[M + H]+. | 124 |
| 4-(9-cyclopropyl-2- (3-methoxy-1H- pyrazol-1-yl)-8- (pyridin-4-yl)-9H- purin-6- yl)morpholine | 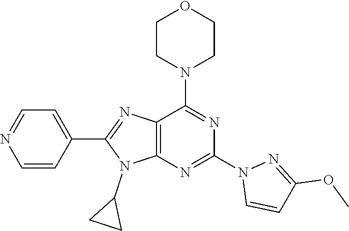 [SMILES: COc1ccn(-c2nc(N3CCOCC3)c3nc(-c4ccncc4)n(C4CC4)c3n2)n1] | ¹H NMR (400 MHz, CDCl3) δ 8.77 (dd, J = 4.6, 1.6 Hz, 2H), 8.42 (d, J = 2.8 Hz, 1H), 7.88 (dd, J = 4.5, 1.6 Hz, 2H), 5.94 (d, J = 2.8 Hz, 1H), 4.40 (bs, 4H), 4.08 (s, 3H), 3.92- 3.84 (m, 4H), 3.53 (pent, J = 3.8 Hz, 1H), 1.25 (q, J = 7.1 Hz, 2H), 1.01-0.94 (m, 2H); LCMS (ESI) m/z: 419.1. [M + H]+. | 125 |
| 4-(9-methyl-8- (pyridin-4-yl)-2-(3- (trifluoromethyl)- 1H-pyrazol-1-yl)- 9H-purin-6- yl)morpholine | 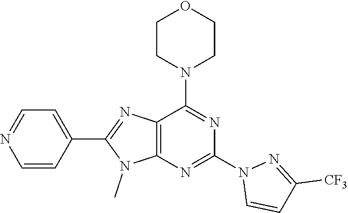 [SMILES: Cn1c(-c2ccncc2)nc2c(N3CCOCC3)nc(-n3ccc(C(F)(F)F)n3)nc21] | ¹H NMR (400 MHz, DMSO-d₆) δ 8.94 (s, 1H), 8.80-8.79 (m, 2H), 7.92 (d, J = 4.8 Hz, 2H), 7.03 (d, J = 2.0 Hz, 1H), 4.63-4.55 (m, 4H), 4.00 (s, 3H), 3.96-3.79 (m, 4H); LCMS (ESI) m/z: 431.1/433.1 [M + H]⁺. | 126 |

### Preparation of (S)-3-methyl-4-(7-((R)-3-methylmorpholino)-2-(4-phenyl-1H-pyrazol-1-yl)thiazolo[5,4-d]pyrimidin-5-yl)morpholine (Compound 127)

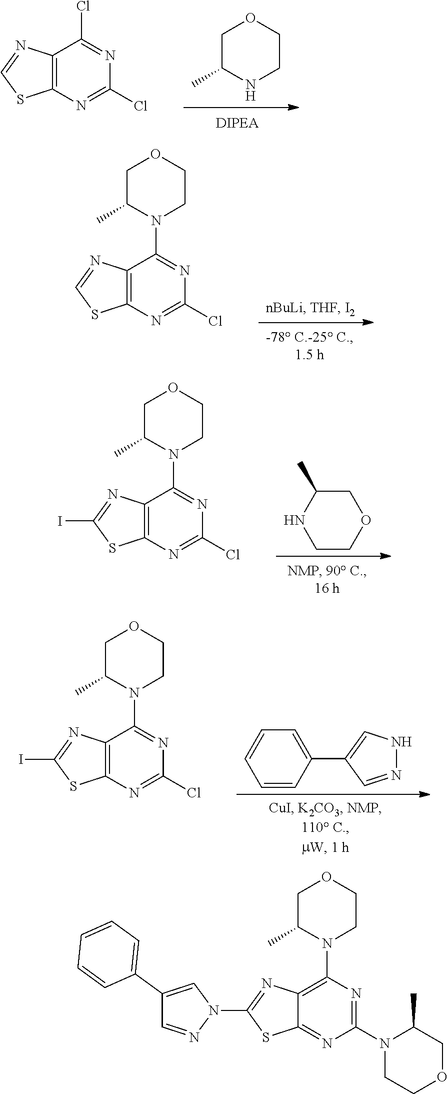

Step 1: Preparation of (R)-4-(5-chlorothiazolo[5,4-d]pyrimidin-7-yl)-3-methylmorpholine. To a stirred solution of 5,7-dichlorothiazolo[5,4-d]pyrimidine (2.00 g, 9.71 mmol) and DIPEA (3.40 mL, 19.4 mmol) in isopropanol (2.9 mL) was added (R)-3-methylmorpholine (1.08 g, 10.7 mmol) dropwise, and the resulting mixture was stirred at 25° C. for 1.5 h. It was concentrated and the residue was triturated with water and the solid formed was collected by filtration, dried under high vacuum to afford product (2.10 g, 80%) as a brown solid. LCMS (ESI) m/z: 271.1 [M+H]⁺.

### Step 2: Preparation of (R)-4-(5-chloro-2-iodothiazolo[5,4-d]pyrimidin-7-yl)-3-methylmorpholine

To a solution of (R)-4-(5-chlorothiazolo[5,4-d]pyrimidin-7-yl)-3-methylmorpholine (1.2 g, 4.43 mmol) in tetrahydrofuran (50 ml) was added n-butyllithium (3.4 ml, 8.5 mmol) at −78° C. and stirred at −78° C. for 0.5 h. Then a solution of iodine (2.25 g, 8.86 mmol) in tetrahydrofuran (10 ml) was added to the reaction mixture and stirred at −78° C.-25° C. for an additional 2 h. The reaction mixture was then quenched with saturated solution of sodium thiosulfate and extracted with ethyl acetate (100 mL*2). The pooled organic phase was washed with brine (100 mL), dried over sodium sulfate and concentrated. The residue was slurried in EA:DCM (15 mL, v/v=10:1) mixture to afford the desire compound (R)-4-(5-chloro-2-iodothiazolo[5,4-d]pyrimidin-7-yl)-3-methylmorpholine as yellow solid (1 g, 2.52 mmol, 57%). LCMS (ESI) m/z: 397.0 [M+H]⁺.

### Step 3: Preparation of (R)-4-(2-iodo-5-((S)-3-methylmorpholino)thiazolo[5,4-d]pyrimidin-7-yl)-3-methylmorpholine

To a solution of (R)-4-(5-chloro-2-iodothiazolo[5,4-d]pyrimidin-7-yl)-3-methylmorpholine (0.5 g, 1.26 mmol) in 1-methyl-2-pyrrolidinone (7 mL) was added (S)-3-methylmorpholine (0.63 g, 6.3 mmol) at 25° C. and the reaction was stirred at 90° C. for 16 h under Ar protection. The mixture was filtered and purified by Prep-HPLC (SunFire C18, 4.6*50 mm, 3.5 um column Xbridge C18 3.5 μm 4.6×50 mm column. The mobile phase was acetonitrile/10 mM formic acid aqueous solution.) to give the desired product as a white solid. (120 mg, 23%) LCMS (ESI) m/z: 462.2 [M+H]+.

### Step 4: Preparation of (S)-3-methyl-4-(7-((R)-3-methylmorpholino)-2-(4-phenyl-1H-pyrazol-1-yl)thiazolo[5,4-d]pyrimidin-5-yl)morpholine

To a solution of (R)-4-(2-iodo-5-((S)-3-methylmorpholino)thiazolo[5,4-d]pyrimidin-7-yl)-3-methylmorpholine (0.08 g, 0.17 mmol) and 4-phenyl-1H-pyrazole (0.038 g, 0.26 mmol) in 1-methyl-2-pyrrolidinone (3 mL) were added potassium carbonate (0.06 g, 0.46 mmol), (1S,2S)-N1,N2-dimethylcyclohexane-1,2-diamine (0.013 g, 0.09 mmol) and cuprous iodide (0.01 g, 0.05 mmol) and the resultant mixture was irradiated with microwave at 110° C. with stirring for 1 h. The mixture was filtered and the crude product was purified by Prep-HPLC (SunFire C18, 4.6*50 mm, 3.5 um column Xbridge C18 3.5 μm 4.6×50 mm column. The mobile phase was acetonitrile/10 mM formic acid aqueous solution) to obtain the desired product (0.0128 g, 16%) as a yellow solid. ¹H NMR (400 MHz, DMSO) δ 9.05 (s, 1H), 8.39 (s, 1H), 7.81 (d, J=7.3 Hz, 2H), 7.44 (t, J=7.6 Hz, 2H), 7.32 (t, J=7.5 Hz, 1H), 5.24 (s, 2H), 4.57 (s, 1H), 4.19 (d, J=12.1 Hz, 1H), 3.99 (d, J=7.9 Hz, 1H), 3.90 (d, J=9.2 Hz, 1H), 3.81-3.69 (m, 3H), 3.58 (s, 2H), 3.46-3.37 (m, 2H), 3.14 (t, J=11.1 Hz, 1H), 1.32 (d, J=6.7 Hz, 3H), 1.19 (d, J=6.7 Hz, 3H); LCMS (ESI) m/z: 478.2 [M+H]⁺.

### Synthesis of 4-(9-((methylsulfonyl)methyl)-2-(1H-pyrazol-1-yl)-8-(pyridin-4-yl)-9H-purin-6-yl)morpholine (Compound 128)

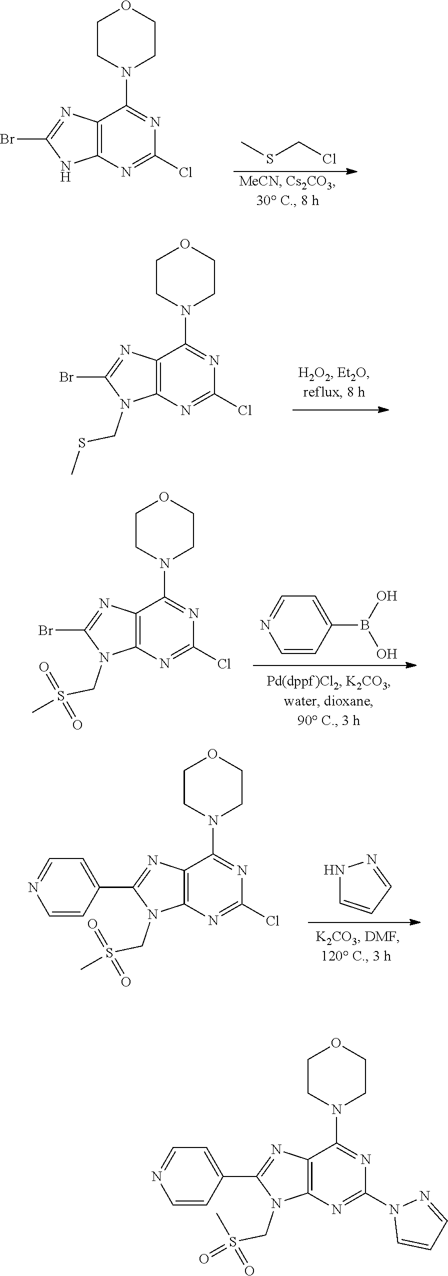

### Step 1: Preparation of 4-(8-bromo-2-chloro-9-((methylthio)methyl)-9H-purin-6-yl)morpholine

To a solution of 4-(8-bromo-2-chloro-9H-purin-6-yl)morpholine (400 mg, 1.26 mmol) in acetonitrile (10 mL) were added (chloromethyl)(methyl)sulfane (183 mg, 1.89 mmol) and cesium carbonate (821 mg, 2.52 mmol). The mixture was stirred at 30° C. for 8 h, quenched with water (15 mL) and extracted with ethyl acetate (20*3 mL). The organic layer was combined, washed with brine (30 mL), dried over sodium sulfate, filtered and concentrated. The residue was purified by flash chromatography on silica gel (petroleum ether:ethyl acetate=75:25) to give the desired product as yellow oil (550 mg, 100%). LCMS (ESI) m/z: 377.7/379.6[M+H]+.

### Step 2: Preparation of 4-(8-bromo-2-chloro-9-((methylsulfonyl)methyl)-9H-purin-6-yl)morpholine

To a solution of 4-(8-bromo-2-chloro-9-((methylthio)methyl)-9H-purin-6-yl)morpholine (550 mg, 1.45 mmol, 1.0 e.q.) in ethoxyethane (10 mL) was added hydrogen peroxide in ethoxyethane (10 mL). The mixture was stirred at 50° C. for 8 h. Then the reaction was quenched with water (15 mL) and extracted with ethyl acetate (20*3 mL). The organic layer was combined, washed with brine (30 mL), dried over sodium sulfate, filtered and concentrated. The residue was purified by flash chromatography on silica gel (petroleum ether:ethyl acetate=75:25) to give product as white solid. (450 mg, 100%).

### Step 3: Preparation of 4-(2-chloro-9-((methylsulfonyl)methyl)-8-(pyridin-4-yl)-9H-purin-6-yl)morpholine

To a solution of 4-(8-bromo-2-chloro-9-((methylsulfonyl)methyl)-9H-purin-6-yl)morpholine (450 mg, 1.10 mmol) in dioxane (10 mL) were added pyridin-4-ylboronic acid (202 mg, 1.65 mmol, 1.5 e.q.), potassium carbonate (305 mg, 2.20 mmol) and dichloro[1,1-bis(diphenylphosphino)ferrocene]palladium(I) dichloromethane adduct (50 mg, 0.196 mmol). The resultant mixture was stirred at 90° C. for 3 h. Then the reaction was quenched with water (15 mL) and extracted with ethyl acetate (20*3 mL). The organic layer was combined, washed with brine (30 mL), dried over sodium sulfate, filtered and concentrated. The residue was purified by flash chromatography on silica gel (petroleum ether:ethyl acetate=75:25) to give product as yellow oil (160 mg, 35.6%). LCMS (ESI) m/z: 408.8 [M+H]+.

### Step 4: Preparation of 4-(9-((methylsulfonyl)methyl)-2-(1H-pyrazol-1-yl)-8-(pyridin-4-yl)-9H-purin-6-yl)morpholine

To a solution of 4-(2-chloro-9-((methylsulfonyl)methyl)-8-(pyridin-4-yl)-9H-purin-6-yl)morpholine (160 mg, 0.392 mmol) in N,N-dimethylformamide (10 mL) were added 1H-pyrazole (266 mg, 3.92 mmol) and potassium carbonate (109 mg, 0.784 mmol). The mixture was stirred at 120° C. for 3 h and concentrated. The residue was purified by Prep-HPLC (0.05% NH4HCO3/H2O:CH3CN=5%-95%) to obtain 4-(9-((methylsulfonyl)methyl)-2-(1H-pyrazol-1-yl)-8-(pyridin-4-yl)-9H-purin-6-yl)morpholine (57.6 mg, 35.6%) as a white solid.

¹H NMR (400 MHz, DMSO-d₆) δ 8.80 (d, J=2.7 Hz, 3H), 7.88 (dd, J=4.5, 1.5 Hz, 2H), 7.80 (d, J=0.8 Hz, 1H), 6.56 (dd, J=2.6, 1.6 Hz, 1H), 5.88 (s, 2H), 4.75-4.00 (m, 4H), 3.80 (t, J=4 Hz, 4H), 3.23 (s, 3H); LCMS (ESI) m/z: 441.0[M+H]+.

The following compounds were synthesized according to the protocol described above:

| Name | Structure | NMR, MS | # |
|---|---|---|---|
| 2-methyl-4-(6- morpholino-2-(1H- pyrazol-1-yl)-8-(pyridin- 4-yl)-9H-purin-9- yl)butan-2-ol | 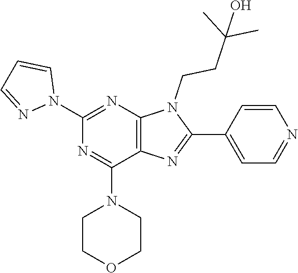 [SMILES: CC(C)(O)CCn1c(-c2ccncc2)nc2c(N3CCOCC3)nc(-n3cccn3)nc21] | ¹H NMR (400 MHz, DMSO) δ 8.78 (d, J = 5.9 Hz, 2H), 8.68 (d, J = 2.5 Hz, 1H), 7.87 (d, J = 6.0 Hz, 2H), 7.79 (s, 1H), 6.62-6.48 (m, 1H), 4.62-4.02 (m, 7H), 3.82-3.73 (m, 4H), 1.85-1.75 (m, 2H), 1.12 (s, 6H); LCMS (ESI) m/z: 435.2[M + H]+. | 129 |
| 4-(9- ((methylsulfonyl)methyl)- 2-(4-phenyl-1H-pyrazol- 1-yl)-8-(pyridin-4-yl)-9H- purin-6-yl)morpholine | 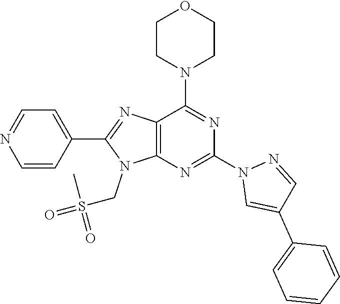 [SMILES: CS(=O)(=O)Cn1c(-c2ccncc2)nc2c(N3CCOCC3)nc(-n3cc(-c4ccccc4)cn3)nc21] | ¹H NMR (400 MHz, DMSO-d₆) δ 9.25 (s, 1H), 8.80 (d, J = 5.8 Hz, 2H), 8.30 (s, 1H), 7.90 (d, J = 5.8 Hz, 2H), 7.80 (d, J = 7.3 Hz, 2H), 7.43 (t, J = 7.6 Hz, 2H), 7.32-7.26 (m, 1H), 5.92 (s, 2H), 4.64-4.50 (m, 2H), 4.25-4.10 (m, 2H), 3.82 (s, 4H), 3.25 (s, 3H); LCMS (ESI) m/z: 516.8[M + H]+. | 130 |

### Synthesis of 4-(8-(cyclohex-1-en-1-yl)-9-methyl-2-(1H-pyrazol-1-yl)-9H-purin-6-yl)morpholine (Compound 131) and 4-(8-cyclohexyl-9-methyl-2-(1H-pyrazol-1-yl)-9H-purin-6-yl)morpholine (Compound 132)

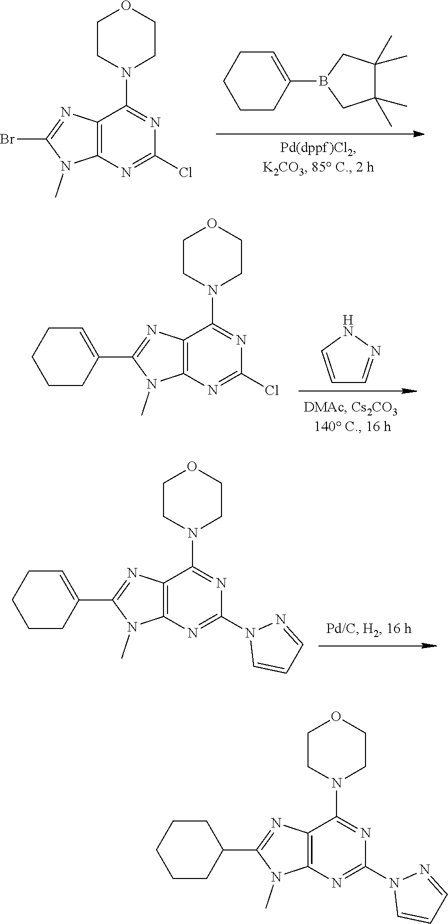 [SMILES: Cn1c(Br)nc2c(N3CCOCC3)nc(Cl)nc21 ; CC1(C)CB(C2=CCCCC2)CC1(C)C ; Cn1c(C2=CCCCC2)nc2c(N3CCOCC3)nc(Cl)nc21 ; c1cn[nH]c1 ; Cn1c(C2=CCCCC2)nc2c(N3CCOCC3)nc(-n3cccn3)nc21 ; Cn1c(C2CCCCC2)nc2c(N3CCOCC3)nc(-n3cccn3)nc21]

### Step 1: 4-(2-chloro-8-(cyclohex-1-en-1-yl)-9-methyl-9H-purin-6-yl)morpholine

A mixture of 4-(8-bromo-2-chloro-9-methyl-9H-purin-6-yl)morpholine (1 g, 3 mmol), 2-(cyclohex-1-en-1-yl)-4,4,5,5-tetramethyl-1,3,2-dioxaborolane (688 mg, 3.3 mmol), [1,1′-bis(diphenylphosphino)ferrocene]dichloropalladium(II) (200 mg, 0.3 mmol) and potassium carbonate (1.1 g, 2.5 mmol) in dioxane (10 mL) and water (0.1 mL) under nitrogen atmosphere was stirred at 85° C. for 16 h. The crude product thus obtained was purified by flash chromatography on silica gel (Petroleum ether/Ethyl acetate 20:1→10:1→5:1) to give the 4-(2-chloro-8-(cyclohex-1-en-1-yl)-9-methyl-9H-purin-6-yl)morpholine (600 mg, 60%) as a white solid. LCMS (ESI) m/z: 333.9 [M+H]⁺.

### Step 2: 4-(8-(cyclohex-1-en-1-yl)-9-methyl-2-(1H-pyrazol-1-yl)-9H-purin-6-yl)morpholine

A mixture of 4-(2-chloro-8-(cyclohex-1-en-1-yl)-9-methyl-9H-purin-6-yl)morpholine (70 mg, 0.21 mmol) and 1H-pyrazole (28.5 mg, 0.42 mmol) and cesium carbonate (205.4 mg, 0.63 mmol) in N,N-dimethylacetamide (1 mL) was stirred at 140° C. for 16 h. The crude product was purified by flash chromatography on silica gel (Dichloromethane/Methanol 20:1→10:1) to give the 4-(8-(cyclohex-1-en-1-yl)-9-methyl-2-(1H-pyrazol-1-yl)-9H-purin-6-yl)morpholine (25 mg, 35%) as a white solid. 1H NMR (400 MHz, CD₃OD) δ 8.65 (d, J=2.4 Hz, 1H), 7.78 (s, 1H), 6.58-6.44 (m, 1H), 6.36 (dd, J=3.8, 1.9 Hz, 1H), 4.35 (s, 4H), 3.85-3.80 (m, 7H), 2.52 (d, J=2.1 Hz, 2H), 2.33 (dd, J=6.2, 2.7 Hz, 2H), 1.88-1.65 (m, 4H); LCMS (ESI) m/z: 366.1 [M+H]⁺.

### Step 3: 4-(8-cyclohexyl-9-methyl-2-(1H-pyrazol-1-yl)-9H-purin-6-yl)morpholine

A mixture of 4-(8-(cyclohex-1-en-1-yl)-9-methyl-2-(1H-pyrazol-1-yl)-9H-purin-6-yl)morpholine (0.1 g, 0.3 mmol), Palladium on carbon (10 mg, 10%) in Methanol (10 mL) under hydrogen was stirred at 85° C. for 16 h. The crude product was purified by flash chromatography on silica gel (Dichloromethane/Methanol 20:1→10:1) to give the 4-(8-cyclohexyl-9-methyl-2-(1H-pyrazol-1-yl)-9H-purin-6-yl)morpholine (64 mg, 64%) as a white solid. 1H NMR (400 MHz, CD₃OD) δ 8.63 (s, 1H), 7.77 (d, J=1.0 Hz, 1H), 6.51 (dd, J=2.6, 1.7 Hz, 1H), 4.34 (s, 4H), 3.92-3.80 (m, 4H), 3.78 (s, 3H), 3.03-2.84 (m, 1H), 1.98 (d, J=13.4 Hz, 2H), 1.93-1.84 (m, 2H), 1.78 (d, J=12.6 Hz, 1H), 1.74-1.60 (m, 2H), 1.56-1.42 (m, 2H), 1.38-1.29 (m, 1H); LCMS (ESI) m/z: 368.0 M+H]⁺.

### Preparation of 3-{1-[8-(3,6-dihydro-2H-pyran-4-yl)-9-methyl-6-(morpholin-4-yl)-9H-purin-2-yl]-1H-pyrazol-3-yl}benzonitrile (Compound 133)

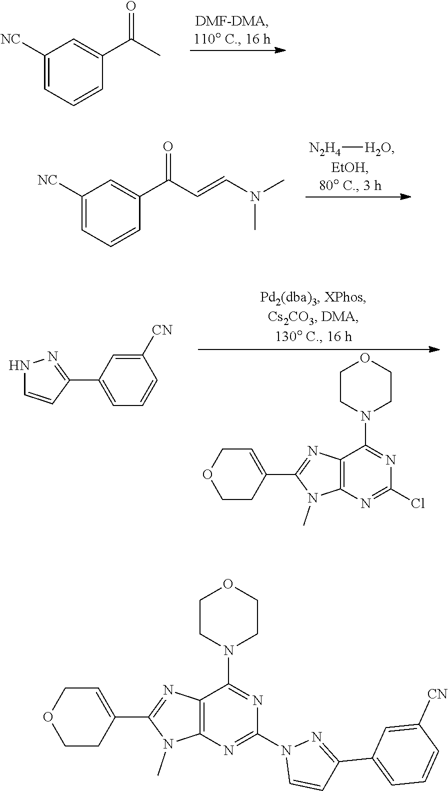

### Step 1: Preparation of (E)-3-(3-(Dimethylamino)acryloyl)benzonitrile

A mixture of 3-acetylbenzonitrile (1.50 g, 10 mmol) in N,N-dimethylformamide dimethyl acetal (10 mL) was stirred at 110° C. for 16 h. The resultant mixture was concentrated to obtain (E)-3-(3-(Dimethylamino)acryloyl)benzonitrile (2.00 g, 10 mmol, 100%) as light yellow oil and was used directly in the next step without further purification. LCMS (ESI) m/z: 201 [M+H]⁺.

### Step 2: Preparation of 3-(1H-pyrazol-3-yl)benzonitrile

A mixture of (E)-3-(3-(Dimethylamino)acryloyl)benzonitrile (2.00 g, 10 mmol) and hydrazine monohydrate (1.50 g, 30 mmol) in ethanol (20 mL) was stirred at 80° C. for 3 h. The resultant mixture was concentrated and the residue was purified by flash column chromatography through silica gel using a gradient of 0-30% ethyl acetate in petroleum ether to obtain 3-(1H-pyrazol-3-yl)benzonitrile (1.40 g, 8.3 mmol, 83%) as a light yellow solid. LCMS (ESI) m/z: 170 [M+H]⁺.

### Step 3: Preparation of 3-(1-(8-(3,6-Dihydro-2H-pyran-4-yl)-9-methyl-6-morpholino-9H-purin-2-yl)-1H-pyrazol-3-yl)benzonitrile

A mixture of 4-(2-chloro-8-(3,6-dihydro-2H-pyran-4-yl)-9-methyl-9H-purin-6-yl)morpholine (200 mg, 0.60 mmol), 3-(1H-pyrazol-3-yl)benzonitrile (128 mg, 0.76 mmol), tris(dibenzylideneacetone) dipalladium (56.0 mg, 0.060 mmol), 2-dicyclohexylphosphino-2′,4′,6′-triisopropylbiphenyl (56.0 mg, 0.12 mmol) and cesium carbonate (392 mg, 1.2 mmol) in N,N-dimethylacetamide (8 mL) was stirred at 130° C. under nitrogen for 16 h. The mixture was cooled to room temperature, quenched with water (10 mL) and extracted with ethyl acetate (3×10 mL). The organic layers were pooled, washed with water and brine (20 mL), dried over sodium sulfate, filtered, and concentrated under reduced pressure. The crude product was purified by prep-HPLC (Boston C18 21*250 mm 10 μm column. The mobile phase was acetonitrile/0.01% aqueous ammonium bicarbonate) to obtain 3-(1-(8-(3,6-Dihydro-2H-pyran-4-yl)-9-methyl-6-morpholino-9H-purin-2-yl)-1H-pyrazol-3-yl)benzonitrile (80.0 mg, 0.17 mmol, 29%) was afforded as white solid. ¹H NMR (400 MHz, Dimethylsulfoxide-d₆) δ 8.78 (d, J=2.7 Hz, 1H), 8.38 (t, J=1.7 Hz, 1H), 8.32 (dt, J=8.0, 1.4 Hz, 1H), 7.85 (dt, J=7.7, 1.4 Hz, 1H), 7.70 (t, J=7.8 Hz, 1H), 7.19 (d, J=2.7 Hz, 1H), 6.57 (dd, J=6.3, 2.1 Hz, 1H), 4.88 (q, J=3.2 Hz, 1H), 4.47-4.15 (−, 4H), 4.10-4.03 (i, 1H), 3.98-23.91 (, 1H), 3.83-3.71 (, 7H), 3.31 (3, 1H), 2.22-2.12 (m, 2H); LCMS(ESI) m/z: 469 [M+H]+.

The following compounds were synthesized according to the protocol described above.

| # | Structure | 1H NMR Data | # |
|---|---|---|---|
| tert-butyl 6-(1-(9- methyl-6- morpholino-8- (pyridin-4-yl)-9H- purin-2-yl)-1H- pyrazol-3-yl)-2- azaspiro[3.3] heptane-2- carboxylate | 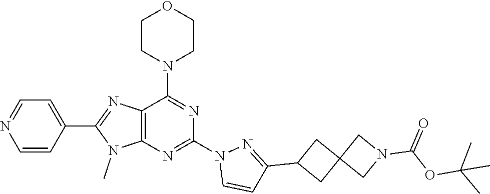 [SMILES: Cn1c(-c2ccncc2)nc2c(N3CCOCC3)nc(-n3ccc(C4CC5(C4)CN(C(=O)OC(C)(C)C)C5)n3)nc21] | ¹H NMR (400 MHz, Dimethylsulfoxide- d₆) δ 8.79-8.77 (m, 2H), 8.60 (d, J = 1.6 Hz, 1H), 7.91-7.90 (m, 2H), 6.44 (d, J = 1.6 Hz, 1H), 4.53-4.00 (m, 4H), 4.01-3.91 (m, 5H), 3.85-3.75 (m, 6H), 3.49-3.41 (m, 1H), 2.59-2.54 (m, 2H), 2.39-2.34 (m, 2H); LCMS (ESI) m/z: 558 [M + H]⁺. | 134 |
| 4-(2-(3-(2- azaspiro[3.3] heptan-6- yl)-1H- pyrazol-1-yl)-9- methyl-8-(pyridin- 4-yl)-9H-purin-6- yl)morpholine | 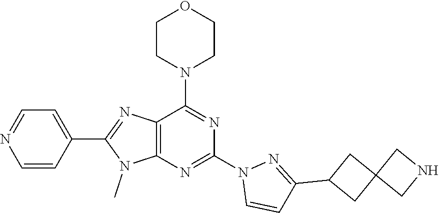 [SMILES: Cn1c(-c2ccncc2)nc2c(N3CCOCC3)nc(-n3ccc(C4CC5(CNC5)C4)n3)nc21] | ¹H NMR (400 MHz, Dimethylsulfoxide- d₆) δ 8.78-8.77 (m, 2H), 8.59 (d, J = 2.0 Hz, 1H), 7.91-7.90 (m, 2H), 6.44 (d, J = 2.0 Hz, 1H), 4.88-3.99 (m, 4H), 3.98-3.88 (m, 4H), 3.83-3.72 (m, 2H), 3.54-3.44 (m, 3H), 2.38-2.24 (m, 2H); LCMS (ESI) m/z: 458 [M + H]⁺. | 135 |
| 4-(9-methyl-2-(3- (1- methylpyrrolidin- 3-yl)-1H-pyrazol- 1-yl)-8-(pyridin-4- yl)-9H-purin-6- yl)morpholine | 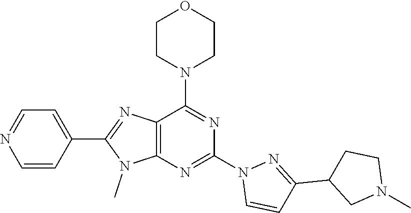 [SMILES: CN1CCC(c2ccn(-c3nc(N4CCOCC4)c4nc(-c5ccncc5)n(C)c4n3)n2)C1] | ¹H NMR (500 MHz, Dimethylsulfoxide- d₆) δ 8.78 (d, J = 6.0 Hz, 2H), 8.61 (d, J = 2.6 Hz, 1H), 8.25 (s, 1H), 7.90 (dd, J = 4.5, 1.6 Hz, 2H), 6.45 (d, J = 2.6 Hz, 1H), 4.26 (s, 4H), 3.93 (s, 3H), 3.80- 3.75 (m, 4H), 3.52 (dt, J = 15.2, 7.5 Hz, 1H), 3.06 (t, J = 8.7 Hz, 1H), 2.79 (dd, J = 15.1, 8.3 Hz, 1H), 2.76-2.65 (m, 2H), 2.42 (s, 3H), 2.32-2.22 (m, 1H), 2.03-1.89 (m, 1H); LCMS (ESI) m/z: 446.2 [M + H]⁺. | 136 |
| 4-(9-methyl-8- (pyridin-4-yl)-2-(3- (pyrrolidin-3-yl)- 1H-pyrazol-1-yl)- 9H-purin-6- yl)morpholine | 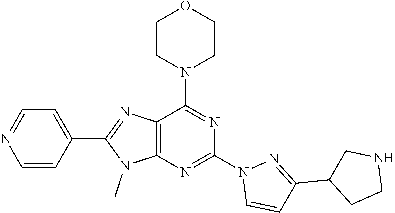 [SMILES: Cn1c(-c2ccncc2)nc2c(N3CCOCC3)nc(-n3ccc(C4CCNC4)n3)nc21] | ¹H NMR (400 MHz, Chloroform-d) δ 8.79 (dd, J = 4.5, 1.6 Hz, 2H), 8.50 (d, J = 2.6 Hz, 1H), 7.74 (dd, J = 4.5, 1.6 Hz, 2H), 6.31 (d, J = 2.6 Hz, 1H), 4.42 (s, 4H), 4.02 (s, 3H), 3.95-3.86 (m, 4H), 3.52 (dd, J = 15.8, 7.6 Hz, 1H), 3.38 (dd, J = 11.0, 7.8 Hz, 1H), 3.17 (m, 1H), 3.06 (m, 2H), 2.38-2.19 (m, 1H), 2.00 (m, 1H); LCMS (ESI) m/z: 432.3 [M + H]⁺. | 137 |
| 3-(1-(9-methyl-6- morpholino-8- (tetrahydro-2H- pyran-4-yl)-9H- purin-2-yl)-1H- pyrazol-3- yl)benzonitrile | 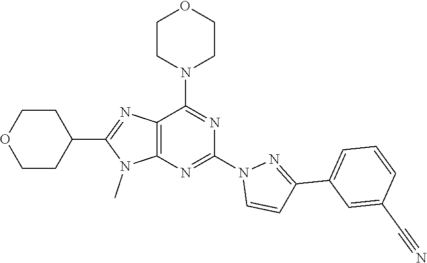 [SMILES: Cn1c(C2CCOCC2)nc2c(N3CCOCC3)nc(-n3ccc(-c4cccc(C#N)c4)n3)nc21] | ¹H NMR (400 MHz, Dimethylsulfoxide- d₆) δ 8.78 (d, J = 2.6 Hz, 1H), 8.38 (t, J = 1.7 Hz, 1H), 8.31 (dt, J = 8.0, 1.5 Hz, 1H), 7.85 (dt, J = 7.7, 1.4 Hz, 1H), 7.69 (t, J = 7.8 Hz, 1H), 7.19 (d, J = 2.7 Hz, 1H), 4.30 (s, 3H), 3.99-3.94 (m, 2H), 3.83-3.74 (m, 7H), 3.51 (td, J = 11.1, 3.4 Hz, 2H), 3.30 (s, 1H), 1.92-1.78 (m, 4H); LCMS (ESI) m/z: 471 [M + H]⁺. | 138 |
| tert-butyl 3-(1-(9- methyl-6- morpholino-8- (pyridin-4-yl)-9H- purin-2-yl)-1H- pyrazol-3- yl)pyrrolidine-1- carboxylate | 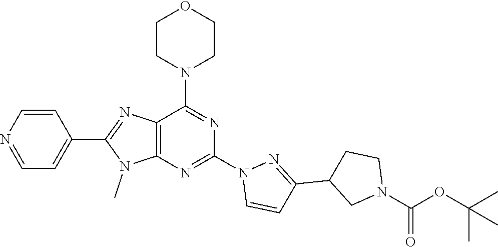 [SMILES: Cn1c(-c2ccncc2)nc2c(N3CCOCC3)nc(-n3ccc(C4CCN(C(=O)OC(C)(C)C)C4)n3)nc21] | ¹H NMR (400 MHz, Chloroform-d) δ 8.80 (dd, J = 4.5, 1.6 Hz, 2H), 8.52 (d, J = 2.5 Hz, 1H), 7.74 (dd, J = 4.5, 1.6 Hz, 2H), 6.32 (d, J = 2.7 Hz, 1H), 4.41 (s, 4H), 4.02 (s, 3H), 3.96-3.71 (m, 5H), 3.65 (m, 2H), 3.52-3.26 (m, 2H), 2.34 (m, 1H), 2.11 (m, 1H), 1.48 (s, 8H); LCMS (ESI) m/z: 532.3 [M + H]⁺. | 139 |

### Synthesis of 4,4′-(9-ethyl-2-(4-phenyl-1H-pyrazol-1-yl)-9H-purine-6,8-diyl)dimorpholine (Compound 140)

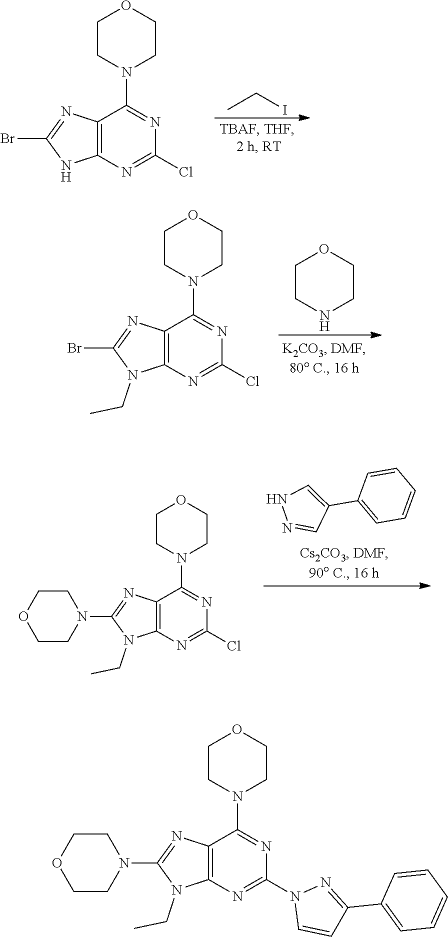

### Step 1: Preparation of 4-(8-bromo-2-chloro-9-ethyl-9H-purin-6-yl)morpholine

To a solution of 4-(8-bromo-2-chloro-9H-purin-6-yl)morpholine (318 mg, 1.0 mmol) in tetrahydrofuran (10 mL) was added tetrabutylammonium fluoride (522 mg, 2.0 mmol). A solution of iodoethane (172 mg, 1.1 mmol) was then added and the reaction mixture was stirred overnight. Water (10 mL) was added and the organic phase separated. The aqueous phrase was extracted with ethyl acetate (20 mL*2). The combined organic layer was dried over sodium sulfate, and concentrated. The residue was purified by silica gel column chromatography (36% ester acetic in petroleum ether) to obtain 4-(8-bromo-2-chloro-9-ethyl-9H-purin-6-yl)morpholine as white solid (150 mg, 73.5%). LCMS (ESI) m/z: 348.0 [M+H]⁺.

### Step 2: Preparation of 4,4′-(2-chloro-9-ethyl-9H-purine-6,8-diyl)dimorpholine

To a solution of 4-(8-bromo-2-chloro-9-ethyl-9H-purin-6-yl)morpholine (640 mg, 1.86 mmol) in N,N-dimethylformamide (10 mL) were added morpholine (324 mg, 3.72 mmol) and potassium carbonate (770 mg, 5.58 mmol, the reaction mixture was stirred at 80° C. for 2 h. The mixture was extracted with ethyl acetate (20 mL*2) and washed with water (10 mL*2). The organic layer was dried over sodium sulfate, and concentrated. The resultant crude product was purified by silica gel column chromatography (50% ester acetic in petroleum ether) to obtain 4,4′-(2-chloro-9-ethyl-9H-purine-6,8-diyl)dimorpholine as white solid (250 mg, 0.39%). LCMS (ESI) m/z: 353.0 [M+H]⁺.

### Step 3: Preparation of 4,4′-(9-ethyl-2-(4-phenyl-1H-pyrazol-1-yl)-9H-purine-6,8-diyl)dimorpholine

To a solution of 4,4′-(2-chloro-9-ethyl-9H-purine-6,8-diyl)dimorpholine (160 mg, 0.45 mmol) in N,N-dimethylformamide (10 mL) were added 4-phenyl-1H-pyrazole (78 mg, 0.54 mmol) and cesium carbonate (439 mg, 1.35 mmol) at 25° C. and the reaction mixture was stirred at 90° C. for 2 h. The resultant mixture was extracted with ethyl acetate (20 mL*2) and washed with water (10 mL*2). The organic layer was dried over sodium sulfate, and concentrated. The residue was purified with prep-HPLC (BOSTON pHlex ODS 10 μm 21.2ᵢÁ250 mm120 A. The mobile phase was acetonitrile/0.1% Ammonium bicarbonate) to obtain 4,4′-(9-ethyl-2-(4-phenyl-1H-pyrazol-1-yl)-9H-purine-6,8-diyl)dimorpholine as white solid (18.6 mg, 8.9%).

¹H NMR (400 MHz, DMSO-d₆) δ 9.01 (s, 1H), 8.21 (d, J=0.7 Hz, 1H), 7.78 (d, J=12 Hz, 2H), 7.41 (t, J=7.7 Hz, 2H), 7.27 (s, 1H), 4.24 (s, 4H), 4.14 (d, J=7.2 Hz, 2H), 3.83-3.72 (m, 8H), 3.25-3.18 (m, 4H), 1.42 (t, J=7.2 Hz, 3H). LCMS (ESI) m/z: 461.0 [M+H]⁺.

### Synthesis of 4-(9-(difluoromethyl)-2-(4-phenyl-1H-pyrazol-1-yl)-8-(pyridin-4-yl)-9H-purin-6-yl)morpholine (Compound 141) and 4-(2-(4-phenyl-1H-pyrazol-1-yl)-8-(pyridin-4-yl)-9H-purin-6-yl)morpholine (Compound 142)

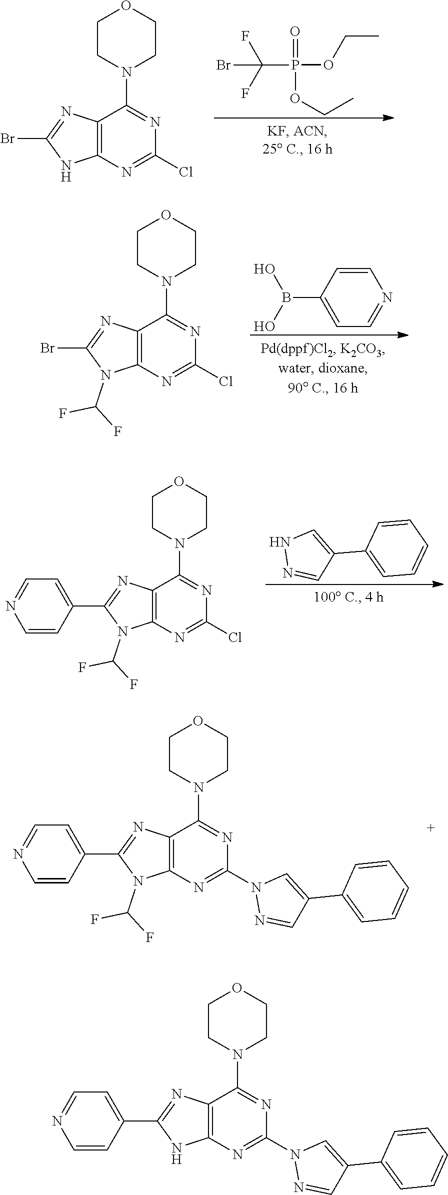

Step 1: Synthesis of 4-(8-bromo-2-chloro-9-(difluoromethyl)-9H-purin-6-yl)morpholine. A mixture of potassium fluoride (174 mg, 3 mmol), 4-(8-bromo-2-chloro-9H-purin-6-yl)morpholine (478 mg, 1.5 mmol), diethyl bromodifluoromethylphosphonate (400 mg, 1.5 mmol) in acetonitrile (15 mL) was stirred at 25° C. for 16 h. The mixture was concentrated and the resultant crude product was purified by column chromatography (SiO2, 100% dichloromethane) to obtain 4-(8-bromo-2-chloro-9-(difluoromethyl)-9H-purin-6-yl)morpholine as yellow solid (600 mg, crude). LCMS, [M+H]+=368.0.

### Step 2: Synthesis of 4-(2-chloro-9-(difluoromethyl)-8-(pyridin-4-yl)-9H-purin-6-yl)morpholine

A solution of 4-(8-bromo-2-chloro-9-(difluoromethyl)-9H-purin-6-yl)morpholine (220 mg, 0.6 mmol), pyridin-4-ylboronic acid (81 mg, 0.66 mmol), 1,1′-bis(diphenylphosphino) ferrocene-palladium(II) dichloride dichloromethane complex (49 g, 0.06 mmol) and potassium carbonate (248 mg, 1.8 mmol) in water (1 mL) and dioxane (9 mL) was stirred at 90° C. for 16 h under argon. The reaction mixture was concentrated and purified by Flash chromatography (Biotage, 40 g silica gel, methanol/dichloromethane=3%-4%) to give the desired product 4-(2-chloro-9-(difluoromethyl)-8-(pyridin-4-yl)-9H-purin-6-yl)morpholine (110 mg, 43%) as yellow solid. LCMS (ESI) m/z 367.0 [M+H]+.

### Step 3: Synthesis of 4-methyl-2-(1-(9-methyl-6-morpholino-8-(pyridin-4-yl)-9H-purin-2-yl)-1H-pyrazol-3-yl)morpholine and 4-(2-(4-phenyl-1H-pyrazol-1-yl)-8-(pyridin-4-yl)-9H-purin-6-yl)morpholine

To a solution of 4-(2-chloro-9-(difluoromethyl)-8-(pyridin-4-yl)-9H-purin-6-yl)morpholine (90 mg, 0.245 mmol) and 4-phenyl-1H-pyrazole (42 mg, 0.294 mmol) and cesium carbonate (240 mg, 0.735 mmol) in dry N,N-dimethylacetamide (5 mL) was stirred at 100° C. for 4 h. The resultant products were purified by pre-HPLC (BOSTON pHlex ODS 10 μm 21.2×250 mm 120 A. The mobile phase was acetonitrile/0.1% Ammonium bicarbonate) to obtain 4-(9-(difluoromethyl)-2-(4-phenyl-1H-pyrazol-1-yl)-8-(pyridin-4-yl)-9H-purin-6-yl)morpholine (68.3 mg, 48%) and 4-(2-(4-phenyl-1H-pyrazol-1-yl)-8-(pyridin-4-yl)-9H-purin-6-yl)morpholine (15.3 mg, 12%) as white solids.

Compound 141: 1H NMR (400 MHz, DMSO-d₆) δ 9.15 (s, 1H), 8.82 (dd, J=4.6, 1.5 Hz, 2H), 8.31 (s, 1H), 8.16 (t, J=57.6 Hz, 1H), 7.83 (dd, J=4.6, 1.5 Hz, 2H), 7.79 (d, J=7.2 Hz, 2H), 7.43 (t, J=7.7 Hz, 2H), 7.29 (t, J=7.4 Hz, 1H), 4.36 (d, 4H), 3.89-3.74 (m, 4H); LCMS, [M+H]+=474.8.

Compound 142: 1H NMR (400 MHz, DMSO-d₆) δ 14.12 (s, 1H), 9.04 (s, 1H), 8.75 (s, 2H), 8.27 (s, 1H), 8.04 (d, J=4.2 Hz, 2H), 7.79 (d, J=7.5 Hz, 2H), 7.42 (t, J=7.6 Hz, 2H), 7.28 (t, J=7.3 Hz, 1H), 4.40 (s, 4H), 3.83 (s, 4H); LCMSA011, [M+H]+=424.8.

### Synthesis of 4-(2-(3-(2,3-dihydrobenzo[b][1,4]dioxin-6-yl)-1H-pyrazol-1-yl)-9-methyl-8-(pyridin-4-yl)-9H-purin-6-yl)morpholine (Compound 143)

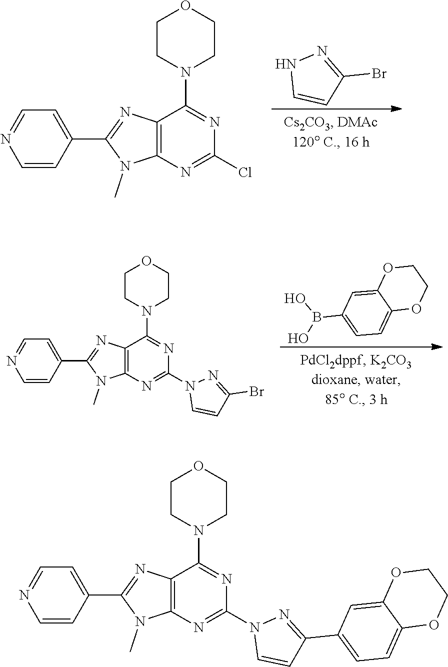 [SMILES: Cn1c(-c2ccncc2)nc2c(N3CCOCC3)nc(Cl)nc21 ; Brc1cc[nH]n1 ; Cn1c(-c2ccncc2)nc2c(N3CCOCC3)nc(-n3ccc(Br)n3)nc21 ; OB(O)c1ccc2c(c1)OCCO2 ; Cn1c(-c2ccncc2)nc2c(N3CCOCC3)nc(-n3ccc(-c4ccc5c(c4)OCCO5)n3)nc21]

### Step 1: Preparation of 4-(2-(3-bromo-1H-pyrazol-1-yl)-9-methyl-8-(pyridin-4-yl)-9H-purin-6-yl)morpholine

A mixture of 4-(2-chloro-9-methyl-8-(pyridin-4-yl)-9H-purin-6-yl)morpholine (100 mg, 0.3 mmol) and 3-bromo-1H-pyrazole (100 mg, 0.3 mmol) and cesium carbonate (0.6 mmol, 150 mg) in N,N-dimethylacetamide (2 mL) was stirred at 120° C. for 16 h. The mixture was quenched with water (10 mL), the resultant precipitate was filtered and the solids were dried to give 4-(2-(3-bromo-1H-pyrazol-1-yl)-9-methyl-8-(pyridin-4-yl)-9H-purin-6-yl)morpholine as a yellow solid. (0.1 g, 75%). LCMS (ESI) m/z: 442.2/443.1 [M+]⁺.

### Step 2: Preparation of 4-(2-(3-(2,3-dihydrobenzo[b][1,4]dioxin-6-yl)-1H-pyrazol-1-yl)-9-methyl-8-(pyridin-4-yl)-9H-purin-6-yl)morpholine

A mixture of 4-(2-(3-bromo-1H-pyrazol-1-yl)-9-methyl-8-(pyridin-4-yl)-9H-purin-6-yl)morpholine (100 mg, 0.22 mmol), (2,3-dihydrobenzo[b][1,4]dioxin-6-yl)boronic acid (180 mg, 0.34 mmol), 1,1′-bis(diphenylphosphino)ferrocene-Palladium(II)dichloride dichloromethane complex (18 mg, 0.1 mmol) and potassium carbonate (91 mg, 0.66 mmol) in dioxane (10 mL)) and water (1 mL) was stirred at 85° C. under nitrogen for 3 h. The mixture was concentrated and purified with Prep-HPLC (BOSTON pHlex ODS 10 μm 21.2×250 mm120 A. The mobile phase was acetonitrile/0.1% Formic acid) to give 4-(2-(3-(2,3-dihydrobenzo[b][1,4]dioxin-6-yl)-1H-pyrazol-1-yl)-9-methyl-8-(pyridin-4-yl)-9H-purin-6-yl)morpholine as a yellow solid. (20 mg, 18%). ¹H NMR (400 MHz, DMSO-d₆) δ 8.79 (dd, J=8.4, 1.2 Hz, 2H), 8.72 (d, J=2.8 Hz, 1H), 7.92 (dd, J=8.4, 1.6 Hz, 2H), 7.44-7.46 (m, 2H), 6.97 (d, J=2.8 Hz, 1H), 6.93-6.95 (m, 1H), 4.13-4.64 (bs, 4H), 4.29 (s, 4H), 3.97 (s, 3H), 3.78-3.81 (m, 4H). LCMS (ESI) m/z: 497.0 [M+H]⁺.

### Synthesis of 4-methyl-2-(1-(9-methyl-6-morpholino-8-(pyridin-4-yl)-9H-purin-2-yl)-1H-pyrazol-3-yl)morpholine (Compound 144)

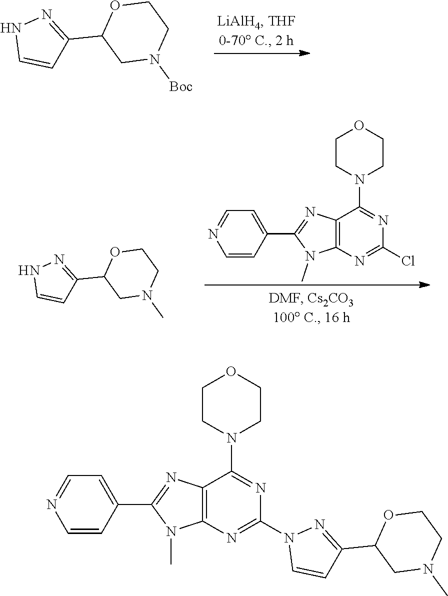 [SMILES: CC(C)(C)OC(=O)N1CCOC(c2cc[nH]n2)C1 ; CN1CCOC(c2cc[nH]n2)C1 ; Cn1c(-c2ccncc2)nc2c(N3CCOCC3)nc(Cl)nc21 ; CN1CCOC(c2ccn(-c3nc(N4CCOCC4)c4nc(-c5ccncc5)n(C)c4n3)n2)C1]

### Step 1: Synthesis of 4-methyl-2-(1H-pyrazol-3-yl)morpholine

A solution of tert-butyl 2-(1H-pyrazol-3-yl)morpholine-4-carboxylate (400 mg, 1.58 mmol) in anhydrous tetrahydrofuran (5 mL) was added to a suspension of lithium aluminum hydride (420 mg, 11 mmol) in anhydrous tetrahydrofuran (15 mL) at 0° C. and stirred under nitrogen for 15 min. The cooling bath was removed and the reaction mixture was gently heated to 70° C. for 2 h. The reaction was quenched by careful addition of sodium sulfate decahydrate with ice bath cooling. Tetrahydrofuran (50 mL) was added to the reaction mixture, stirred for 15 min, filtered and the solid washed with tetrahydrofuran (50 mL). The combined filtrates were evaporated in vacuo. The residue was dissolved in dichloromethane (50 mL), dried with anhydrous sodium sulfate and the solvent removed in vacuo to give 4-methyl-2-(1H-pyrazol-3-yl)morpholine (600 mg, crude) as a yellow oil. LCMS: [M+H]+=168.1.

### Step 2: Synthesis of 4-methyl-2-(1-(9-methyl-6-morpholino-8-(pyridin-4-yl)-9H-purin-2-yl)-1H-pyrazol-3-yl)morpholine

A mixture of 4-methyl-2-(1H-pyrazol-3-yl)morpholine (80 mg, 0.48 mmol) and 4-(2-chloro-9-methyl-8-(pyridin-4-yl)-9H-purin-6-yl)morpholine (80 mg, 0.24 mmol) and cesium carbonate (235 mg, 0.72 mmol) in N,N-dimethylformamide (4 mL) was stirred at 100° C. for 16 h. The crude product was purified by pre-HPLC (BOSTON pHlex ODS 10 μm 21.2×250 mm 120 A. The mobile phase was acetonitrile/0.1% Ammonium bicarbonate) to afford 4-methyl-2-(1-(9-methyl-6-morpholino-8-(pyridin-4-yl)-9H-purin-2-yl)-1H-pyrazol-3-yl) morpholine (40.4 mg, 22%) as white solid.

1H NMR (400 MHz, DMSO-d₆) δ 8.78 (dd, J=4.5, 1.6 Hz, 2H), 8.65 (d, J=2.6 Hz, 1H), 7.91 (dd, J=4.5, 1.6 Hz, 2H), 6.50 (t, J=5.7 Hz, 1H), 4.61 (dd, J=10.3, 2.4 Hz, 1H), 4.33 (s, 4H), 3.94 (s, 3H), 3.89 (d, J=13.1 Hz, 1H), 3.85-3.74 (m, 4H), 3.69 (td, J=11.3, 2.4 Hz, 1H), 2.93 (d, J=11.5 Hz, 1H), 2.68 (d, J=10.9 Hz, 1H), 2.25 (s, 3H), 2.20-2.06 (m, 2H); LCMS: [M+H]+=462.2.

The following compound were synthesized according to the protocol described above:

| Name | Structure | NMR, MS | # |
|---|---|---|---|
| 2-(1-(9-methyl-6- morpholino-8-(1H- pyrazol-3-yl)-9H- purin-2-yl)-1H- pyrazol-3- yl)morpholine | 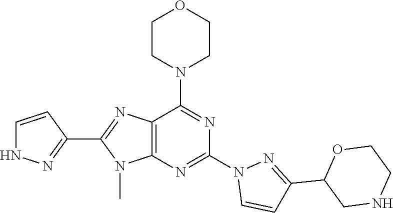 [SMILES: Cn1c(-c2cc[nH]n2)nc2c(N3CCOCC3)nc(-n3ccc(C4CNCCO4)n3)nc21] | 1H NMR (400 MHz, DMSO-d₆) δ 13.41 (s, 1H), 8.61 (d, J = 2.6 Hz, 1H), 7.94 (s, 1H), 6.90 (d, J = 1.8 Hz, 1H), 6.46 (d, J = 2.6 Hz, 1H), 4.52 (dd, J = 10.2, 2.5 Hz, 1H), 4.31 (s, 4H), 4.06 (s, 3H), 3.83 (d, J = 10.9 Hz, 1H), 3.81-3.74 (m, 4H), 3.61 (dt, J = 11.0, 7.3 Hz, 1H), 3.00 (dd, J = 12.4, 2.4 Hz, 1H), 2.83-2.72 (m, 3H); LCMS: [M + H]+ = 437.1. | 145 |

### Synthesis of 4-(9-methyl-2-(3-(1-methylpiperidin-3-yl)-1H-pyrazol-1-yl)-8-(pyridin-4-yl)-9H-purin-6-yl)morpholine (Compound 146)

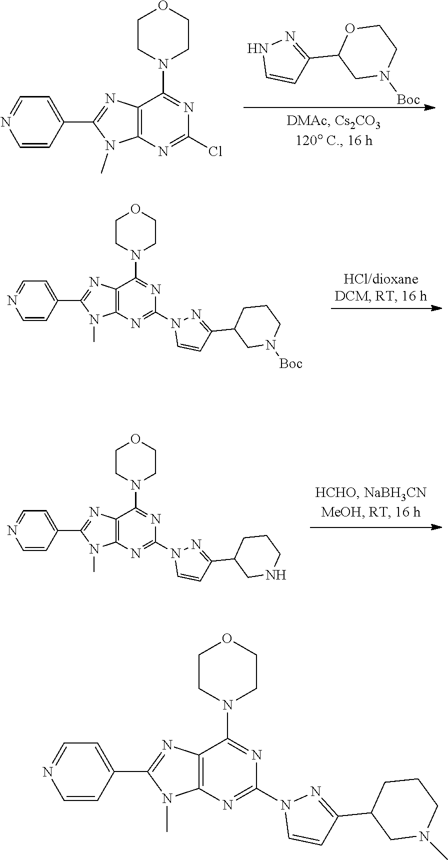 [SMILES: Cn1c(-c2ccncc2)nc2c(N3CCOCC3)nc(Cl)nc21 ; CC(C)(C)OC(=O)N1CCOC(c2cc[nH]n2)C1 ; Cn1c(-c2ccncc2)nc2c(N3CCOCC3)nc(-n3ccc(C4CCCN(C(=O)OC(C)(C)C)C4)n3)nc21 ; Cn1c(-c2ccncc2)nc2c(N3CCOCC3)nc(-n3ccc(C4CCCNC4)n3)nc21 ; CN1CCCC(c2ccn(-c3nc(N4CCOCC4)c4nc(-c5ccncc5)n(C)c4n3)n2)C1]

### Step 1: Preparation of tert-butyl 3-(1-(9-methyl-6-morpholino-8-(pyridin-4-yl)-9H-purin-2-yl)-1H-pyrazol-3-yl)piperidine-1-carboxylate

A mixture of 4-(2-chloro-9-methyl-8-(pyridin-4-yl)-9H-purin-6-yl)morpholine (100 mg, 0.3 mmol) and tert-butyl 3-(1H-pyrazol-3-yl)piperidine-1-carboxylate (150 mg, 0.6 mmol) and cesium carbonate (0.9 mmol, 293 mg) in N,N-dimethylacetamide (4 mL) was stirred at 120° C. for 16 h. The mixture was diluted with water (10 mL) and then filtered. The solid was washed with water (10 mL) and dried to give tert-butyl 3-(1-(9-methyl-6-morpholino-8-(pyridin-4-yl)-9H-purin-2-yl)-1H-pyrazol-3-yl)piperidine-1-carboxylate as a yellow solid (100 mg, 60%). LCMS (ESI) m/z: 546.3 [M+H]+.

### Step 2: Preparation of 4-(9-methyl-2-(3-(piperidin-3-yl)-1H-pyrazol-1-yl)-8-(pyridin-4-yl)-9H-purin-6-yl)morpholine

A mixture of tert-butyl 3-(1-(9-methyl-6-morpholino-8-(pyridin-4-yl)-9H-purin-2-yl)-1H-pyrazol-3-yl)piperidine-1-carboxylate (100 mg, 0.183 mmol) in dichloromethane (4 mL) and hydrochloride/dioxane (4M, 2 mL) was stirred at 25° C. for 16 h. The mixture was extracted with dichloromethane (20 mL*2) and washed with aq. sodium bicarbonate (10 mL*2). The organic layer was dried and concentrated to give 4-(9-methyl-2-(3-(piperidin-3-yl)-1H-pyrazol-1-yl)-8-(pyridin-4-yl)-9H-purin-6-yl)morpholine (80 mg, 97%) as a yellow solid. LCMS (ESI) m/z: 446.2 [M+H]+.

### Step 3: Preparation of 4-(9-methyl-2-(3-(1-methylpiperidin-3-yl)-1H-pyrazol-1-yl)-8-(pyridin-4-yl)-9H-purin-6-yl)morpholine

A solution of 4-(9-methyl-2-(3-(piperidin-3-yl)-1H-pyrazol-1-yl)-8-(pyridin-4-yl)-9H-purin-6-yl)morpholine (80 mg, 0.17 mmol), 37% formaldehyde (5 drops) in methanol (5 mL) was stirred for 1 h. Then sodium cyanoborohydride (56 mg, 0.9 mmol) was added. The reaction mixture was then stirred at room temperature for 16 h. The mixture was concentrated and the residue was purified by Prep-HPLC (BOSTON pHlex ODS 10 μm 21.2×250 mm120 A. The mobile phase was acetonitrile/0.1% Ammonium bicarbonate) to afford 4-(9-methyl-2-(3-(1-methylpiperidin-3-yl)-1H-pyrazol-1-yl)-8-(pyridin-4-yl)-9H-purin-6-yl)morpholine (20 mg, 24%) as white solid. ¹H NMR (400 MHz, CDCl₃) δ 8.80 (dd, J=4.8, 1.6 Hz, 2H), 8.50 (d, J=2.8 Hz, 1H), 7.74 (dd, J=4.4, 1.6 Hz, 2H), 6.33 (d, J=2.4 Hz, 1H), 4.36 (bs, 4H), 3.94 (s, 3H), 3.57-3.79 (m, 4H), 3.37-3.46 (m, 1H), 3.13-3.18 (m, 2H), 2.57-2.61 (m, 1H), 2.52 (s, 3H), 2.36-2.48 (m, 1H), 2.01-2.05 (m, 1H), 1.77-1.85 (m, 2H), 1.49-1.55 (m, 1H); LCMS (ESI) m/z: 460.2 [M+H]+.

### Synthesis of tert-butyl 3-((1-(9-methyl-6-morpholino-8-(pyridin-4-yl)-9H-purin-2-yl)-1H-pyrazol-3-yl)methyl)azetidine-1-carboxylate (Compound 147), 4-(2-(3-(azetidin-3-ylmethyl)-1H-pyrazol-1-yl)-9-methyl-8-(pyridin-4-yl)-9H-purin-6-yl)morpholine (Compound 148) and 4-(9-methyl-2-(3-((1-methylazetidin-3-yl)methyl)-1H-pyrazol-1-yl)-8-(pyridin-4-yl)-9H-purin-6-yl)morpholine (Compound 149)

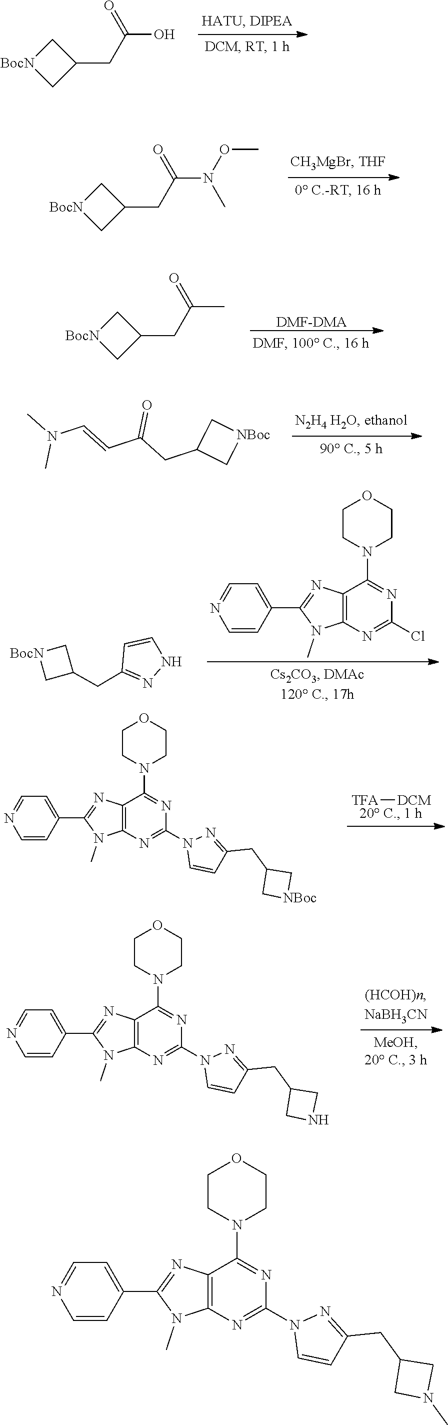

### Step 1: Preparation of tert-butyl 3-(2-(methoxy(methyl)amino)-2-oxoethyl)azetidine-1-carboxylate

A solution of 2-(1-(tert-butoxycarbonyl)azetidin-3-yl)acetic acid (2.15 g, 10 mmol), N,O-dimethylhydroxylamine hydrochloride (1.95 g, 20 mmol), N,N-diisopropylethylamine (5.7 g, 50 mmol) and HATU (5.7 g, 15 mmol) in dichloromethane (50 mL) was stirred at room temperature for 1 h. The mixture was purified by flash (methanol/dichloromethane=1:100) to get tert-butyl 3-(2-(methoxy(methyl)amino)-2-oxoethyl)azetidine-1-carboxylate (1.6 g, 62%) as colorless oil. LCMS (ESI) m/z: 259.2 [M+H]⁺.

### Step 2: Preparation of tert-butyl 3-(2-oxopropyl)azetidine-1-carboxylate

To a solution of tert-butyl 3-(2-(methoxy(methyl)amino)-2-oxoethyl)azetidine-1-carboxylate (1.34 g, 5.1 mmol) in tetrahydrofuran (50 ml) was added methylmagnesium bromide (3M, 2.55 ml, 7.65 mmol) at 0° C. and the mixture was warmed up to 20° C. and stirred for another 16 h. The resultant mixture was quenched with water (2 ml) and dried anhydrous sodium sulfate, filtered and concentrated under reduced pressure. The crude product was purified combi-flash (methanol/dichloromethane: 7:100) to obtain tert-butyl 3-(2-oxopropyl)azetidine-1-carboxylate (1.0 g, 92%) as a yellow oil. LCMS (ESI) m/z: 158.1 [M−55]⁺.

### Step 3: Preparation of (E)-tert-butyl 3-(4-(dimethylamino)-2-oxobut-3-enyl)azetidine-1-carboxylate

A solution of tert-butyl 3-(2-oxopropyl)azetidine-1-carboxylate (1.34 g, 6.28 mmol) and DMAc (10 mL) was stirred at 110° C. for 16 h under argon protection. The resultant mixture was concentrated to obtain (E)-tert-butyl 3-(4-(dimethylamino)-2-oxobut-3-enyl)azetidine-1-carboxylate (1.2 g, 71%). as a yellow solid. LCMS (ESI) m/z: 269.3 [M+H]⁺.

### Step 4: Preparation of tert-butyl 3-((1H-pyrazol-3-yl)methyl)azetidine-1-carboxylate

To a mixture of (E)-tert-butyl 3-(4-(dimethylamino)-2-oxobut-3-enyl)azetidine-1-carboxylate (1.2 g, 4.47 mmol) and hydrazine solution (1.2 mL in water, 87%) was added ethanol (100 mL) and the resultant mixture was stirred at reflux for 5 h. The reaction mixture was concentrated under reduced pressure and the crude product was purified combi-flash (dichloromethane/methanol: 100:7) to obtain tert-butyl 3-((1H-pyrazol-3-yl)methyl)azetidine-1-carboxylate (470 mg, 44%) as a white solid. LCMS (ESI) m/z: 238.2 [M+H]⁺.

### Step 5: Preparation of tert-butyl 3-((1-(9-methyl-6-morpholino-8-(pyridin-4-yl)-9H-purin-2-yl)-1H-pyrazol-3-yl)methyl)azetidine-1-carboxylate

To a solution of 4-(2-chloro-9-methyl-8-(pyridin-4-yl)-9H-purin-6-yl)morpholine (200 mg, 0.60 mmol) in DMA (10 mL) were added tert-butyl 3-((1H-pyrazol-3-yl)methyl)azetidine-1-carboxylate (171 mg, 0.72 mmol) and cesium carbonate (391 mg, 1.2 mmol) and the resultant mixture was stirred at 120° C. under nitrogen for 17 h. It was then filtered, the filtrate was purified by prep-HPLC (BOSTON pHlex ODS 10 μm 21.2×250 mm 120 A. The mobile phase was acetonitrile/0.1% Ammonium bicarbonate) to obtain tert-butyl 3-((1-(9-methyl-6-morpholino-8-(pyridin-4-yl)-9H-purin-2-yl)-1H-pyrazol-3-yl)methyl)azetidine-1-carboxylate (58 mg, 18%) as white solid.

¹H NMR (500 MHz, Chloroform-d) δ 8.82-8.78 (m, 2H), 8.48 (d, J=2.6 Hz, 1H), 7.76-7.72 (m, 2H), 6.23 (d, J=2.6 Hz, 1H), 4.59-4.16 (m, 4H), 4.08 (t, J=8.3 Hz, 2H), 4.02 (s, 3H), 3.94-3.85 (m, 4H), 3.78-3.70 (m, 2H), 3.11-3.04 (m, 2H), 3.01-2.90 (m, 1H), 1.44 (s, 9H). LCMS (ESI) m/z: 532.2 [M+H]⁺.

### Step 6: Preparation of 4-(2-(3-(azetidin-3-ylmethyl)-1H-pyrazol-1-yl)-9-methyl-8-(pyridin-4-yl)-9H-purin-6-yl)morpholine

To a solution of tert-butyl 3-((1-(9-methyl-6-morpholino-8-(pyridin-4-yl)-9H-purin-2-yl)-1H-pyrazol-3-yl)methyl)azetidine-1-carboxylate (49.7 mg, 0.093 mmol) in dichloromethane (3 mL) was added TFA (1 mL) and the mixture was stirred at 20° C. for 1 h. It was concentrated and purified by by PREP-HPLC (BOSTON pHlex ODS 10 μm 21.2×250 mm 120 A. The mobile phase was acetonitrile/0.1% Ammonium bicarbonate) to obtain 4-(2-(3-(azetidin-3-ylmethyl)-1H-pyrazol-1-yl)-9-methyl-8-(pyridin-4-yl)-9H-purin-6-yl)morpholine (29.1 mg, 72%) as a white solid.

¹H NMR (400 MHz, Chloroform-d) δ 8.81 (dd, J=4.4, 1.2 Hz, 2H), 8.47 (d, J=2.6 Hz, 1H), 7.76 (dd, J=4.4, 1.6 Hz, 2H), 6.22 (d, J=2.6 Hz, 1H), 4.73-4.10 (m, 4H), 4.02 (s, 3H), 3.92-3.85 (m, 4H), 3.78 (t, J=7.8 Hz, 2H), 3.56 (t, J=7.3 Hz, 2H), 3.25-3.13 (m, 1H), 3.10-3.05 (m, 2H). LCMS (ESI) m/z: 432.2 [M+H]⁺.

### Step 7: Preparation of 4-(9-methyl-2-(3-((1-methylazetidin-3-yl)methyl)-1H-pyrazol-1-yl)-8-(pyridin-4-yl)-9H-purin-6-yl)morpholine

A mixture of 4-(2-(3-(azetidin-3-ylmethyl)-1H-pyrazol-1-yl)-9-methyl-8-(pyridin-4-yl)-9H-purin-6-yl)morpholine (20 mg, 0.046 mmol) and paraformaldehyde (4 mg, 0.138 mmol) in methanol (5 mL) was stirred at 20° C. for 1 h under nitrogen protection and then sodium cyanoborohydride (14 mg, 0.23 mmol) was added. The resultant mixture was stirred at room temperature for 2 h and added into water (20 mL) slowly and stirred further at room temperature for 5 min. It was extracted with dichloromethane/methanol (10:1) (3×30 mL), the organic phases was dried over sodium sulfate, filtered and concentrated. The crude product thus obtained was purified by by PREP-HPLC (BOSTON pHlex ODS 10 um 21.2×250 mm 120 A. The mobile phase was acetonitrile/0.1% Ammonium bicarbonate) to obtain 4-(9-methyl-2-(3-((1-methylazetidin-3-yl)methyl)-1H-pyrazol-1-yl)-8-(pyridin-4-yl)-9H-purin-6-yl)morpholine (9.6 mg, 43%) as white solid.

¹H NMR (400 MHz, Chloroform-d) δ 8.81 (dd, J=4.8, 1.6 Hz, 2H), 8.47 (d, J=2.6 Hz, 1H), 7.75 (dd, J=4.8, 1.6 Hz, 2H), 6.22 (d, J=2.6 Hz, 1H), 4.80-4.09 (m, 4H), 4.02 (s, 3H), 3.94-3.84 (m, 4H), 3.59 (t, J=7.2 Hz, 2H), 3.07-2.99 (m, 4H), 2.98-2.86 (m, 1H), 2.37 (s, 3H). LCMS (ESI) m/z: 446.2 [M+H]⁺.

### Synthesis of 4-(9-ethyl-2,8-di(1H-pyrazol-1-yl)-9H-purin-6-yl)morpholine (Compound 150)

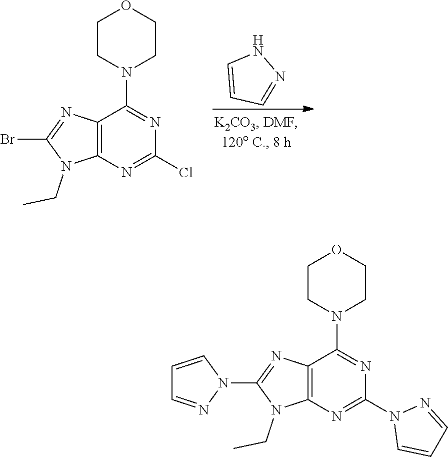 [SMILES: CCn1c(Br)nc2c(N3CCOCC3)nc(Cl)nc21 ; c1cn[nH]c1 ; CCn1c(-n2cccn2)nc2c(N3CCOCC3)nc(-n3cccn3)nc21]

To a solution of 4-(8-bromo-2-chloro-9-ethyl-9H-purin-6-yl)morpholine (180 mg, 0.519 mmol) in N,N-dimethylformamide (10 mL) were added 1H-pyrazole (352 mg, 5.19 mmol) and potassium carbonate (145 mg, 1.038 mmol). The mixture was stirred at 120° C. for 8 h and the resultant mixture was purified by Prep-HPLC (BOSTON pHlex ODS 10 um 21.2×250 mm 120 A. The mobile phase was acetonitrile/0.1% Ammonium bicarbonate) to afford 4-(9-ethyl-2,8-di(1H-pyrazol-1-yl)-9H-purin-6-yl)morpholine (23.3 mg, 12.3%) as white solid.

¹H NMR (400 MHz, DMSO-d₆) δ 8.68 (d, J=2.1 Hz, 1H), 8.51 (d, J=2.3 Hz, 1H), 8.00 (s, 1H), 7.78 (s, 1H), 6.68 (s, 1H), 6.54 (s, 1H), 4.48 (q, J=6.9 Hz, 2H), 4.26 (s, 4H), 3.88-3.60 (m, 4H), 1.32 (t, J=7.0 Hz, 3H); LCMS (ESI) m/z: 365.9[M+H]+.

### Synthesis of 4-(9-cyclopropyl-2-(4-phenyl-1H-pyrazol-1-yl)-8-(pyridin-4-yl)-9H-purin-6-yl)morpholine (Compound 151)

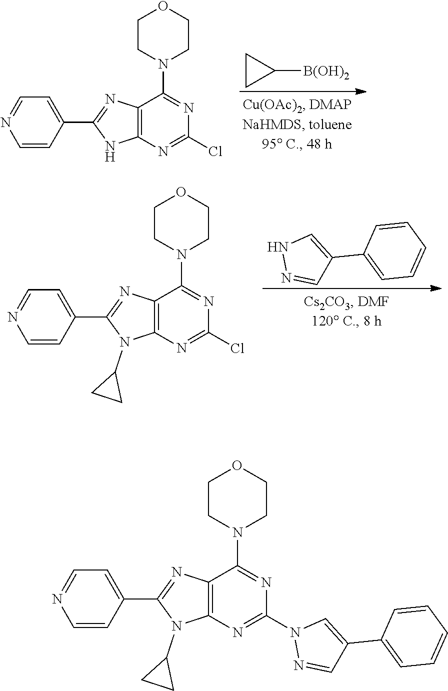 [SMILES: Clc1nc(N2CCOCC2)c2nc(-c3ccncc3)[nH]c2n1 ; OB(O)C1CC1 ; Clc1nc(N2CCOCC2)c2nc(-c3ccncc3)n(C3CC3)c2n1 ; c1ccc(-c2cn[nH]c2)cc1 ; c1ccc(-c2cnn(-c3nc(N4CCOCC4)c4nc(-c5ccncc5)n(C5CC5)c4n3)c2)cc1]

### Step 1: Preparation of 4-(2-chloro-9-cyclopropyl-8-(pyridin-4-yl)-9H-purin-6-yl)morpholine

To a solution of 4-(2-chloro-8-(pyridin-4-yl)-9H-purin-6-yl)morpholine (800.0 mg, 2.53 mmol) in toluene (25 mL) was added cyclopropylboronic acid (432.8 mg, 3.78 mmol), cupric acetate (472.8 mg, 2.53 mmol), DMAP (924.0 mg, 2.53 mmol) and sodium bis(trimethylsilyl)amide (2.52 mL) at 25° C. and the reaction was stirred at 95° C. for 48 h under N₂ protection. The mixture was then extracted with dichloromethane (20 mL*2) and washed with water (10 mL*2). The organic layer was dried over sodium sulfate, and concentrated. The residue was purified by silica gel column chromatography (3% methanol in dichloromethane) to obtain 4-(2-chloro-9-cyclopropyl-8-(pyridin-4-yl)-9H-purin-6-yl)morpholine as white solid (95.0 mg, 10.5%). LCMS (ESI) m/z: 357.1 [M+H]⁺.

### Step 2: Preparation of 4-(9-cyclopropyl-2-(4-phenyl-1H-pyrazol-1-yl)-8-(pyridin-4-yl)-9H-purin-6-yl)morpholine pyrimidine hydrochloride

To a solution of 4-(2-chloro-9-cyclopropyl-8-(pyridin-4-yl)-9H-purin-6-yl)morpholine (95.0 mg, 0.26 mmol) in DMF (3 mL) was added 4-phenyl-1H-pyrazole (56.2 mg, 0.39 mmol) and cesium carbonate (253.5 mg, 0.78 mmol) and the resultant mixture was stirred at 120° C. for 8 h. The mixture was extracted with dichloromethane (20 mL*2) and washed with water (10 mL*2). The organic layer was dried over sodium sulfate, and concentrated. The crude product was purified by prep-HPLC (BOSTON pHlex ODS 10 μm 21.2×250 mm 120 A. The mobile phase was acetonitrile/0.1% Ammonium bicarbonate) to give a white solid. To this solid was added HCl (3M, 0.5 mL) and re-crystallized from water, dried by lyophilization to give the product 4-(9-cyclopropyl-2-(4-phenyl-1H-pyrazol-1-yl)-8-(pyridin-4-yl)-9H-purin-6-yl)morpholine hydrochloride as yellow solid (12.8 mg, 10.6%).

¹H NMR (400 MHz, DMSO-d₆) δ 9.07 (s, 1H), 8.91 (d, J=5.3 Hz, 2H), 8.31 (d, J=5.6 Hz, 2H), 8.29 (s, 1H), 7.80 (d, J=7.7 Hz, 2H), 7.43 (t, J=7.6 Hz, 2H), 7.30 (d, J=7.4 Hz, 1H), 4.25 (s, 4H), 3.86-3.78 (m, 5H), 1.23 (s, 2H), 0.90 (s, 2H); LCMS (ESI) m/z: 465.0 [M+H]⁺.

### Synthesis of 4-(9-ethyl-2-(1H-pyrazol-1-yl)-8-(pyridin-3-yloxy)-9H-purin-6-yl)morpholine (Compound 152)

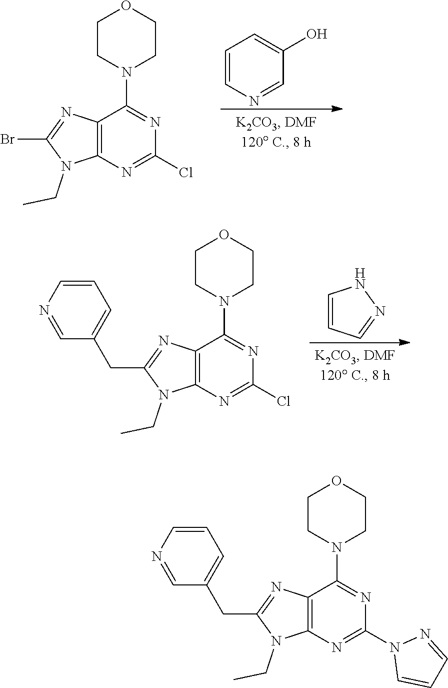 [SMILES: CCn1c(Br)nc2c(N3CCOCC3)nc(Cl)nc21 ; Oc1cccnc1 ; CCn1c(Cc2cccnc2)nc2c(N3CCOCC3)nc(Cl)nc21 ; c1cn[nH]c1 ; CCn1c(Cc2cccnc2)nc2c(N3CCOCC3)nc(-n3cccn3)nc21]

### Step 1: Preparation of 4-(2-chloro-9-ethyl-8-(pyridin-3-yloxy)-9H-purin-6-yl)morpholine

To a solution of 4-(8-bromo-2-chloro-9-ethyl-9H-purin-6-yl)morpholine (360 mg, 1.0 e.q.) in N,N-dimethylformamide (10 mL) was added pyridin-3-ol (108 mg, 1.038 mmol, 1.0 e.q) and potassium carbonate (290 mg, 2.0 e.q.). The resultant mixture was stirred at 120° C. for 8 h. Then the reaction was quenched with water (15 mL) and extracted with ethyl acetate (20*3 mL). The organic layer was combined, washed with brine (30 mL), dried over sodium sulfate, filtered and concentrated. The residue was purified by flash chromatography on silica gel (petroleum ether:ethyl acetate=75:25) to give the desired product as yellow solid (320 mg, 85.6%).

### Step 2: Preparation of 4-(9-ethyl-2-(1H-pyrazol-1-yl)-8-(pyridin-3-yloxy)-9H-purin-6-yl)morpholine

To a solution of 4-(2-chloro-9-ethyl-8-(pyridin-3-yloxy)-9H-purin-6-yl)morpholine (320 mg, 1.0 eq) in N,N-dimethylformamide (10 mL) was added 1H-pyrazole (604 mg, 9.0 mmol) and potassium carbonate (145 mg, 2 eq). The mixture was stirred at 120° C. for 8 h and the product formed was purified by Prep-HPLC (BOSTON pHlex ODS 10 μm 21.2×250 mm 120 A. The mobile phase was acetonitrile/0.1% Ammonium bicarbonate) to afford 4-(9-ethyl-2-(1H-pyrazol-1-yl)-8-(pyridin-3-yloxy)-9H-purin-6-yl)morpholine (6.2 mg, 1.7%) as white solid.

¹H NMR (400 MHz, DMSO-d₆) δ 8.53 (s, 1H), 8.45 (s, 2H), 7.97 (s, 1H), 7.73 (d, J=8.0 Hz, 1H), 7.55-7.42 (m, 1H), 6.65 (s, 1H), 4.29 (d, J=6.9 Hz, 2H), 4.06 (bs, 4H), 3.68 (s, 4H), 1.26 (t, J=8 Hz, 3H); LCMS (ESI) m/z: 392.9 [M+H]+.

### Synthesis of 4-(2-(4-(difluoromethyl)-1H-pyrazol-1-yl)-9-ethyl-8-(pyridin-4-yl)-9H-purin-6-yl)morpholine (Compound 153)

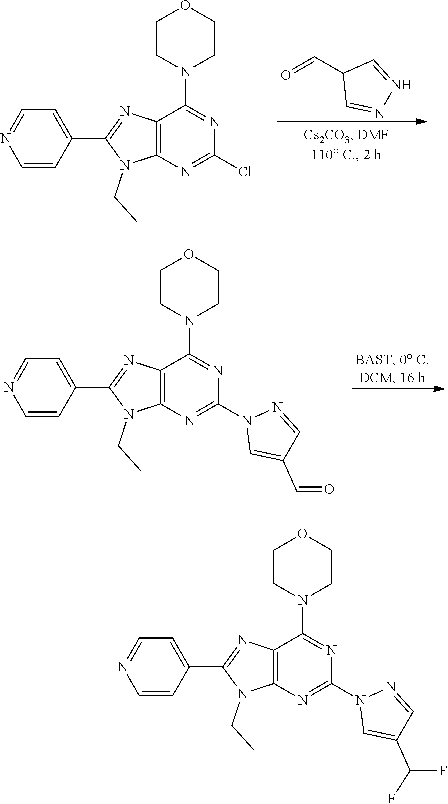

### Step 1: Preparation of 1-(9-ethyl-6-morpholino-8-(pyridin-4-yl)-9H-purin-2-yl)-1H-pyrazole-4-carbaldehyde

To a solution of 4-(2-chloro-9-ethyl-8-(pyridin-4-yl)-9H-purin-6-yl)morpholine (300.0 mg, 0.87 mmol) in N,N-dimethylformamide (10 mL) was added 1H-pyrazole-4-carbaldehyde (125.6 mg, 1.3 mmol) and cesium carbonate (850.2 mg, 2.61 mmol) and the reaction was stirred at 110° C. for 2 h. The mixture was extracted with dichloromethane (20 mL*2) and washed with water (10 mL*2). The organic layer was dried over sodium sulfate, and concentrated. The residue was purified by silica gel column chromatography [(3% methanol in dichloromethane). The mobile phase was acetonitrile/0.1% Ammonium bicarbonate] to give the product 1-(9-ethyl-6-morpholino-8-(pyridin-4-yl)-9H-purin-2-yl)-1H-pyrazole-4-carbaldehyde as yellow solid (260 mg, 73.9%). LCMS (ESI) m/z: 405.1.

### Step 2: Preparation of 4 4-(2-(4-(difluoromethyl)-1H-pyrazol-1-yl)-9-ethyl-8-(pyridin-4-yl)-9H-purin-6-yl)morpholine

To a solution of 1-(9-ethyl-6-morpholino-8-(pyridin-4-yl)-9H-purin-2-yl)-1H-pyrazole-4-carbaldehyde (350.0 mg, 0.86 mmol) in dichloromethane (20 mL) was added bis(2-methoxyethyl)aminosulfur trifluoride (957.9 mg, 4.32 mmol) under N₂ protection and the reaction mixture was stirred at 0° C. for 0.5 h, then warmed to room temperature and stirred for 2 h at 25° C. The mixture was extracted with dichloromethane (20 mL*2) and washed with water (10 mL*2). The organic layer was dried over sodium sulfate, filtered and concentrated. The crude product was purified by pre-HPLC (BOSTON pHlex ODS 10 μm 21.2×250 mm 120 A. The mobile phase was acetonitrile/0.1% Ammonium bicarbonate) to obtain 4-(2-(4-(difluoromethyl)-1H-pyrazol-1-yl)-9-ethyl-8-(pyridin-4-yl)-9H-purin-6-yl)morpholine as yellow solid (19.1 mg, 5.2%).

¹H NMR (400 MHz, DMSO-d₆) δ 9.03 (s, 1H), 8.80 (dd, J=4.5, 1.6 Hz, 2H), 8.03 (s, 1H), 7.84 (dd, J=4.5, 1.6 Hz, 2H), 7.14 (t, J=55.8 Hz, 1H), 4.70-4.10 (m, 6H), 3.90-3.72 (m, 4H), 1.34 (t, J=7.2 Hz, 3H); LCMS (ESI) m/z: 426.8 [M+H]⁺.

### Synthesis of 4-(9-ethyl-2-(3-phenyl-4,5-dihydro-1H-pyrazol-1-yl)-8-(pyridin-4-yl)-9H-purin-6-yl)morpholine (Compound 154)

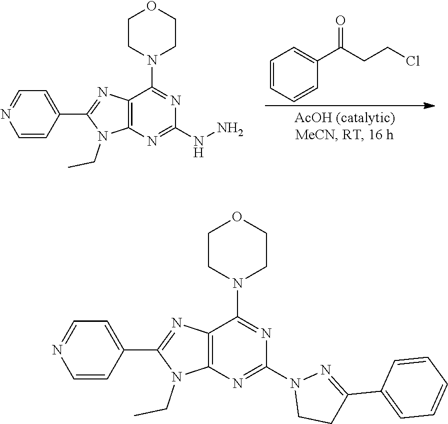 [SMILES: CCn1c(-c2ccncc2)nc2c(N3CCOCC3)nc(NN)nc21 ; O=C(CCCl)c1ccccc1 ; CCn1c(-c2ccncc2)nc2c(N3CCOCC3)nc(N3CCC(c4ccccc4)=N3)nc21]

A mixture of 4-(9-ethyl-2-hydrazineyl-8-(pyridin-4-yl)-9H-purin-6-yl)morpholine (68 mg, 0.2 mmol), 3-chloro-1-phenylpropan-1-one (34 mg, 0.2 mmol) and acetic acid (2 drops) in acetonitrile was stirred at room temperature for 16 h. The mixture was filtered and the filtrate was concentrated. The residue was purified by prep-HPLC (Column Xbridge 21.2*250 mm C18, 10 um, Mobile Phase A: water (10 mmol/L ammonium bicarbonate) B: acetonitrile) to afford 4-(9-ethyl-2-(3-phenyl-4,5-dihydro-1H-pyrazol-1-yl)-8-(pyridin-4-yl)-9H-purin-6-yl)morpholine as pale yellow solid. (15.8 mg, 17%) ¹H NMR (400 MHz, DMSO-d₆) δ 8.75 (d, J=6.0 Hz, 2H), 7.85-7.72 (m, 4H), 7.47 (t, J=7.2 Hz, 2H), 7.41 (t, J=7.2 Hz, 1H), 4.36 (q, J=7.2 Hz, 2H), 4.25 (bs, 4H), 4.15 (t, J=8 Hz, 2H), 3.80-3.71 (m, 4H), 3.27 (d, J=10.4 Hz, 2H), 1.31 (t, J=7.2 Hz, 3H); LCMS (ESI) m/z: 455.0 [M+H]+.

### Synthesis of 4-(2-(4-cyclobutyl-1H-pyrazol-1-yl)-9-ethyl-8-(pyridin-4-yl)-9H-purin-6-yl)morpholine (Compound 155)

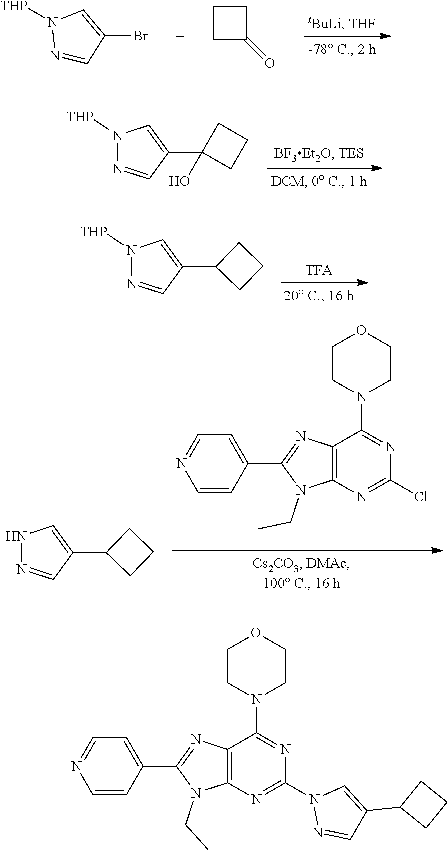

### Step 1: Synthesis of 1-(1-(tetrahydro-2H-pyran-2-yl)-1H-pyrazol-4-yl)cyclobutanol

To a stirred solution of 4-bromo-1-(tetrahydro-2H-pyran-2-yl)-1H-pyrazole (462 mg, 2 mmol) in tetrahydrofuran (20 mL) was added butyl lithium (1.6 M in hexane. 1.88 mL, 2 mmol) at −78° C. And the reaction mixture was stirred 10 minutes. Cyclobutanone (210 mg, 3 mmol) was added drop wise and the reaction mixture was stirred at −78° C. for 2 h. The reaction mixture quenched with addition of saturated aqueous ammonium chloride (10 mL), then diluted with water (20 mL) and extracted with ethyl acetate (50 mL*3). The organic layer was dried over sodium sulfate and the solvent was removed under reduced pressure to afford the crude product which was purified by silica gel chromatography eluting with a linear gradient of 0% to 6% methanol in dichloromethane to get 1-(1-(tetrahydro-2H-pyran-2-yl)-1H-pyrazol-4-yl)cyclobutanol (355 mg, 64%) as a white solid. LCMS: [M+H]*=223.1.

### Step 2: Synthesis of 4-cyclobutyl-1-(tetrahydro-2H-pyran-2-yl)-1H-pyrazole

1-(1-(tetrahydro-2H-pyran-2-yl)-1H-pyrazol-4-yl)cyclobutanol (333 mg, 1.5 mmol) was dissolved in dry dichloromethane (10 mL) together with triethylsilane (348 mg, 3 mmol). The mixture cooled to 0° C., and boron trifluoride etherate was added (426 mg, 3 mmol). The reaction mixture was stirred at 0° C. for 1 h, quenched by the addition of water (30 mL) and then diluted and extracted with dichloromethane (50 mL*2). The organic layer was dried over sodium sulfate and the solvent was removed under reduced pressure to afford the crude product. It was purified by silica gel chromatography eluting with a linear gradient of 0% to 20% ethyl acetate in petroleum ether to get 4-cyclobutyl-1-(tetrahydro-2H-pyran-2-yl)-1H-pyrazole (250 mg, crude) as yellow oil. LCMS: [M+H]*=207.0.

### Step 3: Synthesis of 4-cyclobutyl-1H-pyrazole

A mixture of 4-cyclobutyl-1-(tetrahydro-2H-pyran-2-yl)-1H-pyrazole (42 mg, 0.2 mmol) in trifluoroacetic acid (2 mL) was stirred at 20° C. for 16 h. The mixture was concentrated to afford the crude product 4-cyclobutyl-1H-pyrazole (75 mg, crude) as yellow oil, which was directly used for the next step without further purification. LCMS: [M+H]*=123.2.

### Step 4: Synthesis of 4-(2-(4-cyclobutyl-1H-pyrazol-1-yl)-9-ethyl-8-(pyridin-4-yl)-9H-purin-6-yl)morpholine

A mixture of 4-cyclobutyl-1H-pyrazole (50 mg, 0.2 mmol), 4-(2-chloro-9-ethyl-8-(pyridin-4-yl)-9H-purin-6-yl)morpholine (69 mg, 0.2 mmol) and cesium carbonate (196 mg, 0.6 mmol) in N,N-dimethylformamide (5 mL) was stirred at 100° C. for 16 h. The mixture was purified by pre-HPLC (BOSTON pHlex ODS 10 μm 21.2×250 mm 120 A. The mobile phase was acetonitrile/0.1% Ammonium bicarbonate) to afford product 4-(2-(4-cyclobutyl-1H-pyrazol-1-yl)-9-ethyl-8-(pyridin-4-yl)-9H-purin-6-yl)morpholine (30.4 mg, 23.5%) as a white solid.

¹H NMR (400 MHz, DMSO-d₆) δ 8.79 (dd, J=4.6, 1.4 Hz, 2H), 8.48 (s, 1H), 7.84 (dd, J=4.5, 1.5 Hz, 2H), 7.70 (s, 1H), 4.75-4.05 (m, 6H), 3.87-3.70 (m, 4H), 3.46 (pent, J=4.4 Hz, 1H), 2.36-2.25 (m, 2H), 2.16-2.01 (m, 2H), 1.93 (dpent, J=9.2, 2.4 Hz, 2H), 1.33 (t, J=7.2 Hz, 3H); LCMS: [M+H]⁺=430.8.

### Synthesis of 1-(1-(9-ethyl-6-morpholino-8-(pyridin-4-yl)-9H-purin-2-yl)-1H-pyrazol-4-yl)cyclobutan-1-ol (Compound 156)

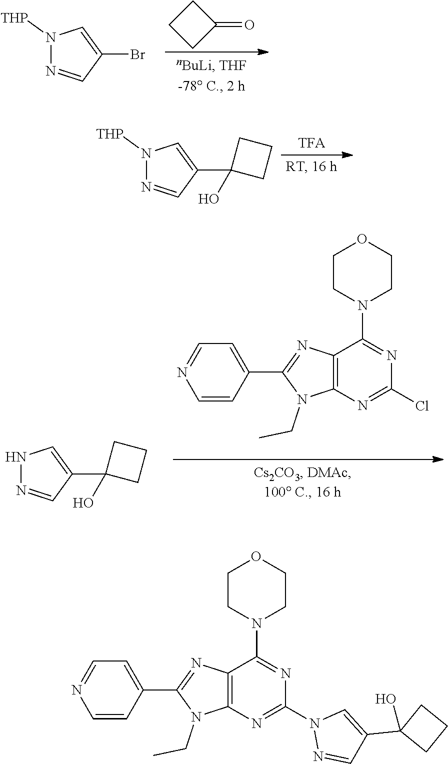

### Step 1: Synthesis of 1-(1-(tetrahydro-2H-pyran-2-yl)-1H-pyrazol-4-yl)cyclobutan-1-ol

To a stirred solution of 4-bromo-1-(tetrahydro-2H-pyran-2-yl)-1H-pyrazole (462 mg, 2 mmol) in tetrahydrofuran (20 mL) was added n-butyllithium (2.5 M in hexane, 0.8 mL, 2 mmol) at −78° C. and the reaction mixture was stirred for 10 minutes. Cyclobutanone (210 mg, 3 mmol) was added drop wise to the mixture and the reaction mixture was stirred at −78° C. for another 2 h. It was then quenched with saturated aqueous ammonium chloride solution (10 mL), diluted with water (20 mL) and extracted with ethyl acetate (50 mL*3). The combined organic layer was dried over sodium sulfate, filtered and concentrated under reduced pressure to afford a residue, which was purified by silica gel chromatography eluting with a linear gradient of 0% to 6% methanol in dichloromethane to get 1-(1-(tetrahydro-2H-pyran-2-yl)-1H-pyrazol-4-yl)cyclobutanol (260 mg, 58.5%) as white solid. LCMS: [M+H]*=223.1.

### Step 2: Synthesis of (1H-pyrazol-4-yl)cyclobutan-1-ol

A mixture of 1-(1-(tetrahydro-2H-pyran-2-yl)-1H-pyrazol-4-yl)cyclobutan-1-ol (200 mg, 0.9 mmol) in trifluoroacetic acid (4 mL) was stirred at room temperature for 16 h. It was concentrated to afford 1-(1H-pyrazol-4-yl) cyclobutan-1-ol (100 mg, crude) as yellow oil. It was directly used for the next step without further purification. LCMS: [M+H]*=139.2.

### Step 3: Synthesis of 1-(1-(9-ethyl-6-morpholino-8-(pyridin-4-yl)-9H-purin-2-yl)-1H-pyrazol-4-yl)cyclobutan-1-ol

A solution of 4-(2-chloro-9-ethyl-8-(pyridin-4-yl)-9H-purin-6-yl)morpholine (206 mg, 0.6 mmol), 1-(1H-pyrazol-4-yl)cyclobutan-1-ol (84 mg, 0.6 mmol) and cesium carbonate (782 mg, 2.4 mmol) in dry N,N-dimethylaniline (10 mL) was stirred at 100° C. for 16 h. The reaction mixture was quenched by the addition of saturated aqueous ammonium chloride solution (20 mL) and water (30 mL) and extracted with ethyl acetate (50 mL*3). The combined organic layer was concentrated and the residue obtained was purified by pre-HPLC (BOSTON pHlex ODS 10 μm 21.2×250 mm 120 A. The mobile phase was acetonitrile/0.1% Ammonium bicarbonate) to afford 1-(1-(9-ethyl-6-morpholino-8-(pyridin-4-yl)-9H-purin-2-yl)-1H-pyrazol-4-yl)cyclobutan-1-ol (12.5 mg, 5%) as yellow solid.

1H NMR (400 MHz, DMSO-d₆) δ 8.79 (d, J=6.0 Hz, 2H), 8.53 (s, 1H), 7.84 (d, J=6.1 Hz, 2H), 7.79 (s, 1H), 5.52 (s, 1H), 4.41 (q, J=7.1 Hz, 2H), 4.35-4.05 m, 4H), 3.82-3.76 (m, 4H), 2.36-2.28 (m, 4H), 1.81 (s, 1H), 1.68 (dd, J=19.4, 8.5 Hz, 1H), 1.33 (t, J=7.2 Hz, 3H); LC-MS: [M+H]+=446.8.

### Synthesis of tert-butyl 3-((1-(9-methyl-6-morpholino-8-(pyridin-4-yl)-9H-purin-2-yl)-1H-pyrazol-3-yl)methyl)azetidine-1-carboxylate (Compound 157)

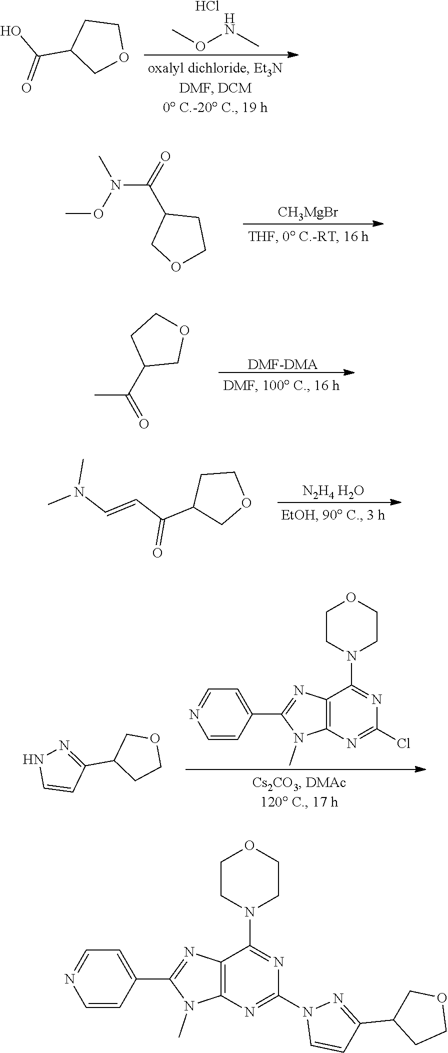

### Step 1: Preparation of N-methoxy-N-methyltetrahydrofuran-3-carboxamide

To a solution of tetrahydrofuran-3-carboxylic acid (986 mg, 8.5 mmol) in dichloromethane (20 ml) was added oxalyl dichloride (1.2 g, 9.35 mmol) at 0° C. followed by 3 drops of N,N-dimethylformamide. After 2 h of stirring at 20° C., the reaction mixture was concentrated under reduced pressure. To the obtained residue 20 ml of chloroform and N,O-dimethylhydroxylamine hydrochloride (1.24 g, 12.75 mmol) were added and the mixture was cooled to 3° C. Triethylamine (2.6 g, 25.5 mmol) was then added dropwise and the mixture was warmed up to room temperature and stirred further for 17 h. The mixture was then acidified with 1 N hydrochloric acid and extracted with chloroform. The organic layer was dried over anhydrous sodium sulfate and concentrated under reduced pressure. The crude residue was purified combi-flash (methanol/dichloromethane: 7:100) to give the crude desired product N-methoxy-N-methyltetrahydrofuran-3-carboxamide (900 mg, 66%) as a yellow oil. ¹H NMR (400 MHz, Chloroform-d) δ 4.06 (t, J=8.4 Hz, 1H), 3.92-3.77 (m, 3H), 3.71 (s, 3H), 3.48-3.36 (m, 1H), 3.21 (s, 3H), 2.28-2.18 (m, 1H), 2.13-2.02 (m, 1H). LCMS (ESI) m/z: 160.2 [M+H]⁺.

### Step 2: Preparation of 1-(tetrahydrofuran-3-yl)ethenone

To a solution of N-methoxy-N-methyltetrahydrofuran-3-carboxamide (900 mg, 5.65 mmol) in THE (50 ml) was added methylmagnesium bromide (2.8 ml, 3M, 8.5 mmol) at 0° C. and the resultant mixture was stirred at 20° C. for 16 h. The resultant mixture was quenched with water (2 ml) and dried anhydrous sodium sulfate, filtered and concentrated. The crude residue was purified combi-flash (methanol/dichloromethane: 10:100) to obtain 1-(tetrahydrofuran-3-yl)ethanone (400 mg, 62%) as yellow oil. ¹H NMR (400 MHz, Chloroform-d) δ 3.98-3.83 (m, 3H), 3.83-3.72 (m, 1H), 3.27-3.13 (m, 1H), 2.21 (s, 3H), 2.16-2.07 (m, 2H). LCMS (ESI) m/z: 115.2 [M+H]⁺.

### Step 3: Preparation of (E)-3-(dimethylamino)-1-(tetrahydrofuran-3-yl)prop-2-en-1-one

A solution of 1-(tetrahydrofuran-3-yl)ethanone (400 mg, 3.5 mmol) and DMAc (4 mL) was stirred at 110° C. for 16 h under argon protection. The mixture was concentrated to obtain (E)-3-(dimethylamino)-1-(tetrahydrofuran-3-yl)prop-2-en-1-one (560 mg, 95%) as yellow oil. LCMS (ESI) m/z: 170.2 [M+H]⁺.

### Step 4: Preparation of 3-(tetrahydrofuran-3-yl)-1H-pyrazole

To a mixture of (E)-3-(dimethylamino)-1-(tetrahydrofuran-3-yl)prop-2-en-1-one (560 mg, 3.3 mmol) and hydrazine solution (0.5 mL) was added ethanol (20 mL) and the resultant mixture was stirred at reflux for 3 h. The reaction mixture was concentrated under reduced pressure and the crude product was purified combi-flash (dichloromethane/methanol: 100:7) to obtain 3-(tetrahydrofuran-3-yl)-1H-pyrazole (220 mg, 48%) as yellow oil. LCMS (ESI) m/z: 139.2 [M+H]⁺.

### Step 5: Preparation of 4-(9-methyl-8-(pyridin-4-yl)-2-(3-(tetrahydrofuran-3-yl)-1H-pyrazol-1-yl)-9H-purin-6-yl)morpholine

To a solution of 4-(2-chloro-9-methyl-8-(pyridin-4-yl)-9H-purin-6-yl)morpholine (198 mg, 0.60 mmol) in N—N-dimethylacetamide (5 mL) were added 3-(tetrahydrofuran-3-yl)-1H-pyrazole (100 mg, 0.72 mmol) and cesium carbonate (586 mg, 1.8 mmol) and the resultant mixture was stirred at 120° C. under nitrogen for 17 h. The resultant mixture was filtered and the filtrate was purified by prep-HPLC (BOSTON pHlex ODS 10 μm 21.2×250 mm 120 A. The mobile phase was acetonitrile/0.1% Ammonium bicarbonate) to obtain 4-(9-methyl-8-(pyridin-4-yl)-2-(3-(tetrahydrofuran-3-yl)-1H-pyrazol-1-yl)-9H-purin-6-yl)morpholine (98.6 mg, 38%) as white solid.

¹H NMR (400 MHz, Chloroform-dt) b 8.81 (d, J=6 Hz, 2H), 8.52 (d, J=2.7 Hz, 1H), 7.76 (d, J=4 Hz, 2H), 6.35 (d, J=2.7 Hz, 1H), 4.93-3.99 (m, 9H), 3.97-3.83 (m, 6H), 3.76 (pent, J=8 Hz, 1H), 2.50-2.35 (m, 1H), 2.21-2.06 (in, 1H). LCMS (ESI) m/z: 433.2 [M+H]+.

The following compounds were synthesized according to the protocols described above:

| Name | Structure | 1H NMR Data | # |
|---|---|---|---|
| 4-(9-ethyl-2- (3-phenyl-1H- pyrazol-1-yl)- 8-(pyridin-4- yl)-9H-purin- 6-yl) morpholine | 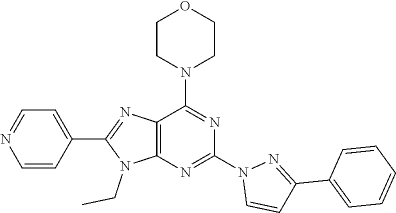 [SMILES: CCn1c(-c2ccncc2)nc2c(N3CCOCC3)nc(-n3ccc(-c4ccccc4)n3)nc21] | ¹H NMR (400 MHz, Dimethylsulfoxide-d₆) δ 8.79 (dd, J = 11.1, 4.3 Hz, 3H), 8.08-7.95 (m, 2H), 7.86 (dd, J = 4.5, 1.6 Hz, 2H), 7.48 (t, J = 7.5 Hz, 2H), 7.40 (d, J = 7.3 Hz, 1H), 7.06 (d, J = 2.7 Hz, 1H), 4.46 (m, 6H), 3.86- 3.59 (m, 4H), 1.36 (t, J = 7.2 Hz, 3H); LCMS (ESI) m/z: 453.2 [M + H]⁺. | 158 |
| 4-(8-(pyridin- 4-yl)-2-(3- (tetrahydro-2H- pyran-4-yl)-1H- pyrazol-1-yl)- 9H-purin-6- yl)morpholine | 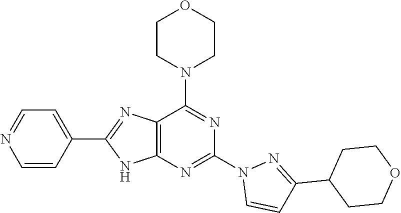 [SMILES: c1cc(-c2nc3c(N4CCOCC4)nc(-n4ccc(C5CCOCC5)n4)nc3[nH]2)ccn1] | ¹H NMR (400 MHz, Dimethylsulfoxide-d₆) δ 8.76-8.70 (m, 2H), 8.54 (d, J = 2.6 Hz, 1H), 8.04- 7.99 (m, 2H), 6.42 (d, J = 2.6 Hz, 1H), 4.35 (s, 4H), 3.97-3.89 (m, 2H), 3.80 (t, J = 4.7 Hz, 4H), 3.46 (td, J = 11.6, 2.2 Hz, 2H), 2.96-2.88 (m, 1H), 1.93-1.85 (m, 2H), 1.77-1.64 (m, 2H); LCMS (ESI) m/z: 433 [M + H]⁺. | 159 |
| 4-(9-methyl- 8-(1- methylpiperidin- 4-yl)-2-(3- phenyl-1H- pyrazol-1-yl)- 9H-purin-6- yl)morpholine | 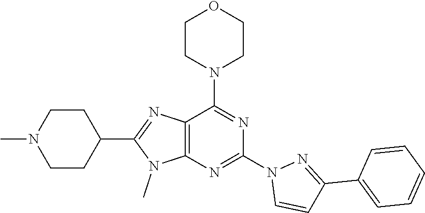 [SMILES: CN1CCC(c2nc3c(N4CCOCC4)nc(-n4ccc(-c5ccccc5)n4)nc3n2C)CC1] | ¹H NMR (400 MHz, Dimethylsulfoxide-d₆) δ 8.72 (d, J = 2.0 Hz, 1H), 7.97-7.95 (m, 2H), 7.50 (t, 2H), 7.02 (d, J = 2.0 Hz, 1H), 4.33-4.25 (m, 4H), 7.76(s, 7H), 2.94- 2.87(m, 3H), 2.22 (s, 3H), 2.07-2.01(m, 2H), 1.88-1.80 (m, 4H); LCMS (ESI) m/z: 459 [M + H]⁺. | 160 |
| 4-(9-methyl-2- (3-phenyl-1H- pyrazol-1-yl)-8- (piperidin-3-yl)- 9H-purin-6- yl)morpholine | 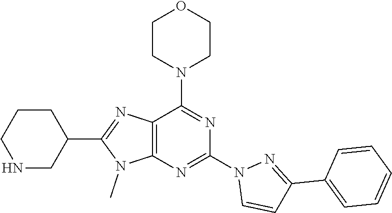 [SMILES: Cn1c(C2CCCNC2)nc2c(N3CCOCC3)nc(-n3ccc(-c4ccccc4)n3)nc21] | ¹H NMR (400 MHz, Dimethylsulfoxide-d₆) δ 8.72 (d, J = 2.6 Hz, 1H), 7.96 (d, J = 7.2 Hz, 2H), 7.47 (t, J = 7.5 Hz, 2H), 7.38 (t, J = 7.3 Hz, 1H), 7.02 (d, J = 2.6 Hz, 1H), 4.29 (s, 4H), 3.77 (s, 7H), 3.24 (d, J = 13.8 Hz, 1H), 3.12 (s, 1H), 3.02 (d, J = 11.5 Hz, 1H), 2.78 (t, J = 11.2 Hz, 1H), 2.65 (d, J = 16.7 Hz, 1H), 2.06 (d, J = 11.0 Hz, 1H), 1.75 (t, J = | 161 |
| | | 12.2 Hz, 2H), 1.57 (d, | |
| | | J = 12.5 Hz, 1H); LCMS | |
| | | (ESI) m/z: 445.2 [M + H]⁺. | |
| 4-(9-methyl-2- (3-phenyl-1H- pyrazol-1-yl)-8- (piperidin-4-yl)- 9H-purin-6- yl)morpholine | 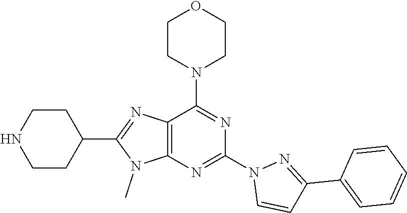 [SMILES: Cn1c(C2CCNCC2)nc2c(N3CCOCC3)nc(-n3ccc(-c4ccccc4)n3)nc21] | ¹H NMR (400 MHz, Dimethylsulfoxide-d₆) δ 8.72 (d, J = 2.0 Hz, 1H), 7.95 (d, J = 5.6 Hz, 2H), 7.48-7.45 (m, 2H), 7.39- 7.36 (m, 1H), 7.02 (d, J = 2.0 Hz, 1H), 4.31- 4.27 (m, 4H), 3.77 (s, 7H), 3.33 (s, 2H), 3.12- 3.07 (m, 2H), 2.77-2.75 (m, 1H), 1.97-1.90 (m, 2H), 1.77-1.75 (m, 2H); LCMS (ESI) m/z: 445 [M + H]⁺. | 162 |
| 4-(9-methyl-2- (3-phenyl-1H- pyrazol-1-yl)-8- (tetrahydro-2H- pyran-4-yl)-9H- purin-6- yl)morpholine | 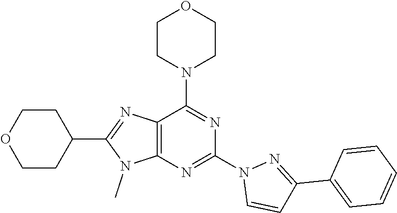 [SMILES: Cn1c(C2CCOCC2)nc2c(N3CCOCC3)nc(-n3ccc(-c4ccccc4)n3)nc21] | ¹H NMR (400 MHz, Dimethylsulfoxide-d₆) δ 8.72 (d, J = 2.7 Hz, 1H), 7.99-7.93 (m, 2H), 7.47 (dd, J = 8.3, 7.0 Hz, 2H), 7.41-7.35 (m, 1H), 7.02 (d, J = 2.7 Hz, 1H), 4.30 (s, 4H), 3.99-3.94 (m, 2H), 3.80-3.75 (m, 7H), 3.51 (td, J = 11.3, 3.2 Hz, 2H), 3.31-3.25 (m, 1H), 1.92-1.77 (m, 4H); LCMS (ESI) m/z: 446 [M + H]⁺. | 163 |
| 4-(8-(3,6- dihydro-2H- pyran-4-yl)-9- methyl-2-(3- phenyl-1H- pyrazol-1-yl)- 9H-purin-6- yl)morpholine | 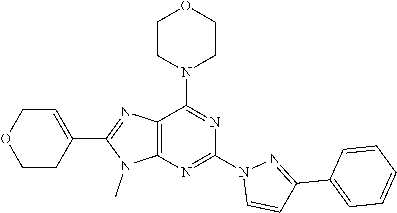 [SMILES: Cn1c(C2=CCOCC2)nc2c(N3CCOCC3)nc(-n3ccc(-c4ccccc4)n3)nc21] | ¹H NMR (400 MHz, Dimethylsulfoxide-d₆) δ 8.72 (d, J = 2.7 Hz, 1H), 7.99-7.90 (m, 2H), 7.47 (t, J = 7.6 Hz, 2H), 7.38 (t, J = 7.4 Hz, 1H), 7.02 (d, J = 2.6 Hz, 1H), 6.57 (dd, J = 6.2, 2.1 Hz, 1H), 4.88 (dd, J = 6.3, 3.3 Hz, 1H), 4.43-4.17 (m, 4H), 4.09-4.04 (m, 1H), 3.96- 3.91 (m, 1H), 3.82- 3.72 (m, 7H), 3.30 (s, 1H), | 165 |
| | | 2.24-2.11 (m, 2H); LCMS | |
| | | (ESI) m/z: 444 [M + H]⁺. | |
| 4-(9-methyl- 2-(3-(1- methylpiperidin- 3-yl)-1H- pyrazol-1-yl)-8- (tetrahydro-2H- pyran-4-yl)-9H- purin-6- yl)morpholine | 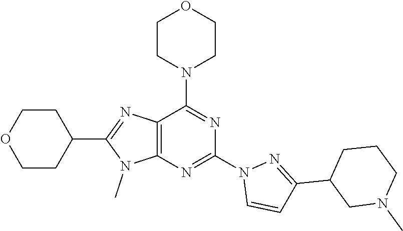 [SMILES: CN1CCCC(c2ccn(-c3nc(N4CCOCC4)c4nc(C5CCOCC5)n(C)c4n3)n2)C1] | ¹H NMR (400 MHz, Dimethylsulfoxide-d₆) δ 8.54 (d, J = 2.6 Hz, 1H), 6.39 (d, J = 2.7 Hz, 1H), 4.26 (s, 4H), 4.00-3.92 (m, 2H), 3.74 (d, J = 4.3 Hz, 7H), 3.50 (td, J = 11.1, 3.5 Hz, 3H), 3.09-3.01 (m, 1H), 3.00-2.82 (m, 2H), 2.29 (s, 3H), 2.14 (t, J = 11.0 Hz, 1H), 2.04 (t, J = 11.6 Hz, 1H), 2.00- 1.91 (m, 1H), 1.90-1.78 (m, 4H), 1.77-1.59 (m, 2H), 1.46-1.36 | 166 |
| | | (m, 1H); LCMS (ESI) | |
| | | m/z: 467 [M + H]⁺. | |
| 4-(9-methyl-2- (3-(piperidin- 3-yl)-1H- pyrazol-1-yl)- 8-(tetrahydro- 2H-pyran-4- yl)-9H-purin- 6-yl) morpholine | 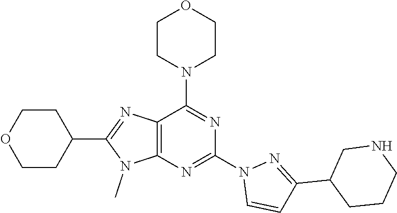 | ¹H NMR (400 MHz, Dimethylsulfoxide-d₆) δ 8.58-8.49 (m, 1H), 6.47- 6.31 (m, 1H), 4.26 (s, 4H), 4.00-3.91 (m, 2H), 3.80-3.70 (m, 7H), 3.50 (td, J = 11.2, 3.5 Hz, 2H), 3.26 (dd, J = 10.2, 4.8 Hz, 2H), 3.10 (d, J = 12.5 Hz, 1H), 2.93 (d, J = 12.2 Hz, 1H), 2.77 (s, 1H), 2.62-2.53 (m, 2H), 1.99 (d, J = 11.5 Hz, 1H), 1.90- 1.75 (m, 4H), 1.69-1.42 (m, 3H); LCMS (ESI) | 167 |
| | | m/z: 453 [M + H]+. | |
| 4-(9-methyl-2- (3-(piperidin- 3-yl)-1H- pyrazol-1-yl)- 8-(pyridin-4- yl)-9H-purin-6- yl)morpholine | 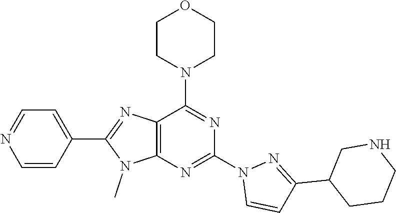 [SMILES: Cn1c(-c2ccncc2)nc2c(N3CCOCC3)nc(-n3ccc(C4CCCNC4)n3)nc21] | ¹H NMR (400 MHz, Dimethylsulfoxide-d₆) δ 8.83-8.74 (m, 2H), 8.58 (d, J = 2.6 Hz, 1H), 7.95- 7.87 (m, 2H), 6.40 (d, J = 2.6 Hz, 1H), 3.93 (s, 8H), 3.78 (t, J = 4.8 Hz, 4H), 3.11 (d, J = 12.0 Hz, 1H), 2.93 (d, J = 12.3 Hz, 1H), 2.85-2.55 (m, 3H), 2.01 (d, J = 12.0 Hz, 1H), 1.77-1.35 (m, 3H); LCMS (ESI) m/z: 446 [M + H]+. | 168 |
| 4-(9-methyl- 2-(3-(1- methylpiperidin- 4-yl)-1H- pyrazol-1-yl)- 8-(pyridin- 4-yl)-9H- purin-6- yl)morpholine | 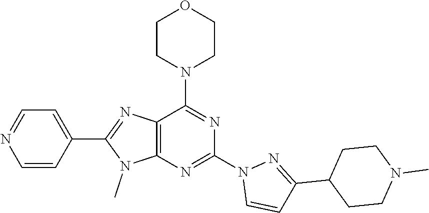 [SMILES: CN1CCC(c2ccn(-c3nc(N4CCOCC4)c4nc(-c5ccncc5)n(C)c4n3)n2)CC1] | ¹H NMR (400 MHz, Dimethylsulfoxide-d₆) δ 8.79-8.77 (m, 2H), 8.59 (d, J = 2.4 Hz, 1H), 7.91- 7.90 (m, 2H), 6.42 (d, J = 2.4 Hz, 1H), 5.50- 4.00 (m, 4H), 3.93 (s, 3H), 3.79-3.77 (m, 4H), 2.91-2.82 (m, 2H), 2.70- 2.59 (m, 1H), 2.21 (s, 3H), 2.08-1.97 (m, 2H), 1.93-1.84 (m, 2H), 1.76- 1.62 (m, 2H); LCMS (ESI) m/z: 460 | 169 |
| | | [M + H]⁺. | |
| 4-(9-methyl- 2-(3- (pyrimidin-5- yl)-1H- pyrazol-1-yl)- 8-(tetrahydro- 2H-pyran-4- yl)-9H- purin-6- yl)morpholine | 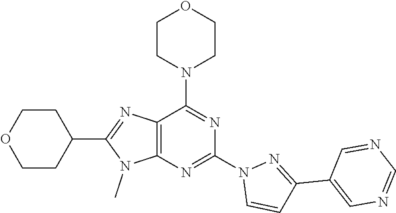 [SMILES: Cn1c(C2CCOCC2)nc2c(N3CCOCC3)nc(-n3ccc(-c4cncnc4)n3)nc21] | ¹H NMR (400 MHz, Dimethylsulfoxide-d₆) δ 9.34 (s, 2H), 9.20 (s, 1H), 8.83 (d, J = 2.7 Hz, 1H), 7.24 (d, J = 2.7 Hz, 1H), 4.31 (s, 4H), 4.03- 3.92 (m, 2H), 3.78 (d, J = 4.7 Hz, 7H), 3.51 (td, J = 11.2, 3.4 Hz, 2H), 3.29-3.25 (m, 1H), 1.96-1.74 (m, 4H); LCMS (ESI) m/z: 448 [M + H]⁺. | 171 |
| 4-(9-methyl- 2-(3- (piperidin-4- yl)-1H- pyrazol-1-yl)- 8-(pyridin-4- yl)-9H- purin-6- yl)morpholine | 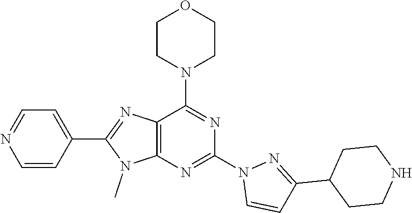 [SMILES: Cn1c(-c2ccncc2)nc2c(N3CCOCC3)nc(-n3ccc(C4CCNCC4)n3)nc21] | 1H NMR (400 MHz, Dimethylsulfoxide-d6) δ 8.79-8.77 (m, 2H), 8.59 (d, J = 2.4 Hz, 1H), 7.91- 7.90 (m, 2H), 6.40 (d, J = 2.4 Hz, 1H), 4.37- 4.28 (m, 4H), 3.93 (s, 3H), 3.79-3.77 (m, 4 H), 3.04-3.01 (m, 2H), 2.81- 2.58 (m, 3H), 1.85-1.82 (m, 2H), 1.61-1.51 (m, 2 H); LCMS (ESI) m/z: 446 [M + H]⁺. | 172 |
| 4-(2-(3- (piperidin-3- yl)-1H- pyrazol-1-yl)- 8-(pyridin-4- yl)-9H- purin-6- yl)morpholine | 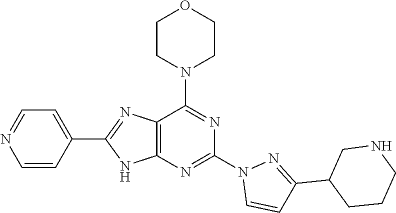 [SMILES: c1cc(-c2nc3c(N4CCOCC4)nc(-n4ccc(C5CCCNC5)n4)nc3[nH]2)ccn1] | ¹H NMR (400 MHz, Dimethylsulfoxide-d₆) δ 8.66 (d, J = 5.2 Hz, 2H), 8.52 (d, J = 2.6 Hz, 1H), 8.05-7.99 (m, 2H), 6.39 (d, J = 2.6 Hz, 1H), 4.34 (s, 4H), 3.11 (d, J = 12.1 Hz, 2H), 2.97-2.62 (m, 4H), 2.06 (s, 1H), 1.75 (s, 1H), 1.67-1.61 (m, 2H); LCMS (ESI) m/z: 432 [M + H]⁺. | 173 |
| 4-(2-(3- isopropyl-1H- pyrazol-1-yl)- 9-methyl-8- (pyridin-4- yl)-9H- purin-6- yl)morpholine | 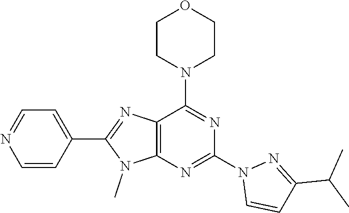 [SMILES: CC(C)c1ccn(-c2nc(N3CCOCC3)c3nc(-c4ccncc4)n(C)c3n2)n1] | ¹H NMR (400 MHz, Dimethylsulfoxide-d₆) δ 8.79-8.77 (m, 2H), 8.59 (d, J = 2.8 Hz, 1H), 7.91-7.90 (m, 2H), 6.41 (d, J = 2.8 Hz, 1H), 4.39- 4.22 (m, 4H), 3.93 (s, 1H), 3.79-3.77 (m, 4H), 3.06-3.00 (m, 1H), 1.27- 1.26 (m, 6H); LCMS (ESI) m/z: 405 [M + H]⁺. | 174 |
| 4-(9-methyl-8- (pyridin-4-yl)- 2-(3- (tetrahydro- 2H-pyran-4-yl)- 1H-pyrazol-1- yl)-9H- purin-6- yl)morpholine | 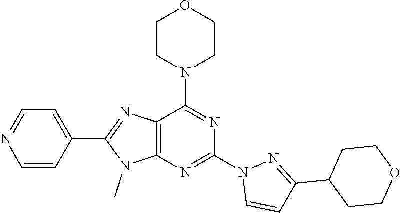 [SMILES: Cn1c(-c2ccncc2)nc2c(N3CCOCC3)nc(-n3ccc(C4CCOCC4)n3)nc21] | ¹H NMR (400 MHz, Dimethylsulfoxide-d₆) δ 8.79-8.77(m, 2H), 8.61 (d, J = 2.4 Hz, 1H), 7.91- 7.89 (m, 2H), 6.44 (d, J = 2.8 Hz, 1H), 4.62- 3.99 (m, 4H), 3.95-3.93 (m, 5H), 3.79-3.76 (t, 4H), 3.49 (t, 2H), 2.96 (t, 1H), 1.86-1.83 (m, 2H), 1.75-1.69 (m, 2H); LCMS (ESI) m/z: 447 [M + H]⁺. | 175 |
| tert-butyl 4- (1-(9- methyl-6- morpholino-8- (pyridin-4- yl)-9H- purin-2-yl)- 1H-pyrazol-3- yl)piperidine- 1- carboxylate | 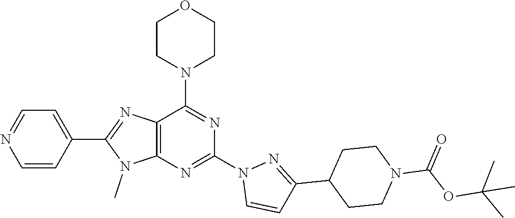 | ¹H NMR (400 MHz, Dimethylsulfoxide-d₆) δ 8.79-8.77 (m, 2H), 8.59 (d, J = 2.4 Hz, 1H), 7.91- 7.90 (m, 2H), 6.42 (d, J = 2.4 Hz, 1H), 5.50- 4.00 (m, 4H), 3.93 (s, 3H), 3.79-3.77 (m, 4H), 2.91-2.82 (m, 2H), 2.70-2.59 (m, 1H), 2.21 (s, 3H), 2.08-1.97 (m, 2H), 1.93-1.84 (m, 2H), 1.76-1.62 (m, 2H); LCMS (ESI) m/z: | 176 |
| | | 460 [M + H]⁺. | |
| 4-(9-methyl-8- (pyridin-4-yl)-2- (3-(pyridin-4- yl)-1H-pyrazol- 1-yl)-9H- purin-6- yl)morpholine | 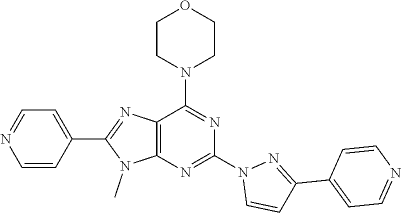 [SMILES: Cn1c(-c2ccncc2)nc2c(N3CCOCC3)nc(-n3ccc(-c4ccncc4)n3)nc21] | ¹H NMR (400 MHz, Chloroform-d) δ 8.81 (d, J = 4.8 Hz, 2H), 8.69-8.68 (m, 3H), 7.78 (d, J = 4.4 Hz, 2H), 7.76 (d, J = 4.8 Hz, 2H), 6.88 (d, J = 2.4 Hz, 1H), 4.43 (bs, 4H), 4.06 (s, 3H), 3.93-3.91 (m, 4H); LCMS (ESI) m/z: 440.2 [M + H]⁺. | 177 |
| 4-(9-methyl-8- (pyridin-4-yl)-2- (3-(pyrimidin-5- yl)-1H-pyrazol- 1-yl)-9H- purin-6- yl)morpholine | 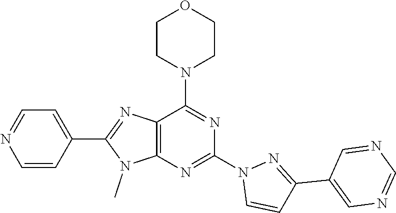 [SMILES: Cn1c(-c2ccncc2)nc2c(N3CCOCC3)nc(-n3ccc(-c4cncnc4)n3)nc21] | ¹H NMR (400 MHz, DMSO) δ 9.36 (s, 2H), 9.21 (s, 1H), 8.89 (d, J = 2.7 Hz, 1H), 8.79 (d, J = 6.0 Hz, 2H), 7.93 (dd, J = 4.5, 1.6 Hz, 2H), 7.27 (d, J = 2.7 Hz, 1H), 4.37 (s, 4H), 3.98 (s, 3H), 3.85- 3.76 (m, 4H); LCMS (ESI) m/z: 441.2 [M + H]⁺. | 178 |
| 4-(2-(3-(3- fluorophenyl)- 1H-pyrazol-1- yl)-9-methyl-8- (pyridin-4-yl)- 9H-purin-6- yl)morpholine | 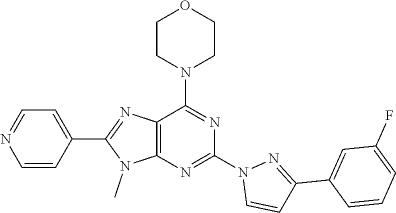 [SMILES: Cn1c(-c2ccncc2)nc2c(N3CCOCC3)nc(-n3ccc(-c4cccc(F)c4)n3)nc21] | ¹H NMR (400 MHz, Chloroform-d) δ 8.81 (d, J = 4.4 Hz, 2H), 8.63 (d, J = 2.8 Hz, 1H), 7.76-7.72 (m, 4H), 7.41-7.39 (m, 1H), 7.05-7.04 (m, 1H), 6.78 (d, J = 2.4 Hz, 1H), 4.43 (bs, 4H), 4.05 (s, 3H), 3.93-3.90 (m, 4H); LCMS (ESI) m/z: 457.1 [M + H]⁺. | 179 |
| tert-butyl 3-(1-(9- methyl-6- morpholino-8- (pyridin-4-yl)- 9H-purin-2-yl)- 1H-pyrazol-3- yl)piperidine-1- carboxylate | 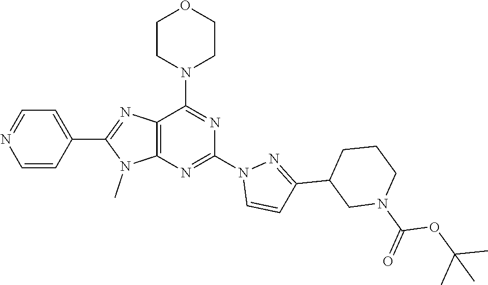 [SMILES: Cn1c(-c2ccncc2)nc2c(N3CCOCC3)nc(-n3ccc(C4CCCN(C(=O)OC(C)(C)C)C4)n3)nc21] | ¹H NMR (400 MHz, Dimethylsulfoxide-d₆) δ 8.83-8.74 (m, 2H), 8.62 (d, J = 2.5 Hz, 1H), 7.95- 7.86 (m, 2H), 6.45 (d, J = 2.5 Hz, 1H), 3.94 (s, 9H), 3.78 (t, J = 4.8 Hz, 4H), 3.00-2.70 (m, 3H), 2.04 (d, J = 12.2 Hz, 1H), 1.75-1.62 (m, 2H), 1.50-1.45 (m, 1H), 1.42 (s, 9H); LCMS (ESI) m/z: 546 [M + H]⁺. | 180 |
| 4-(9-ethyl-2-(5- methyl-4,5,6,7- tetrahydro-2H- pyrazolo[4,3- c]pyridin-2-yl)- 8-(pyridin-4- yl)-9H- purin-6- yl)morpholine | 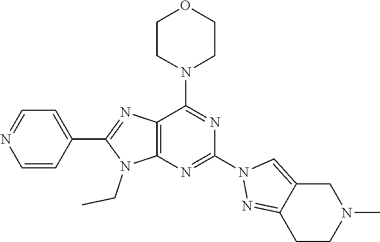 | ¹H NMR (400 MHz, DMSO) δ 8.79 (dd, J = 4.5, 1.6 Hz, 2H), 8.40 (s, 1H), 7.83 (dd, J = 4.5, 1.6 Hz, 2H), 4.69-4.07 (m, 6H), 3.89-3.64 (m, 4H), 3.43 (s, 2H), 2.77 (t, J = 5.6 Hz, 2H), 2.66 (t, J = 5.7 Hz, 2H), 2.37 (s, 3H), 1.32 (t, J = 7.2 Hz, 3H); LCMS (ESI) m/z: 445.8 [M + H]+. | 181 |

### Synthesis of 4-(9-ethyl-8-(pyridin-4-yl)-2-(3-(tetrahydro-2H-pyran-4-yl)-1H-pyrazol-1-yl)-9H-purin-6-yl)morpholine (compound 182)

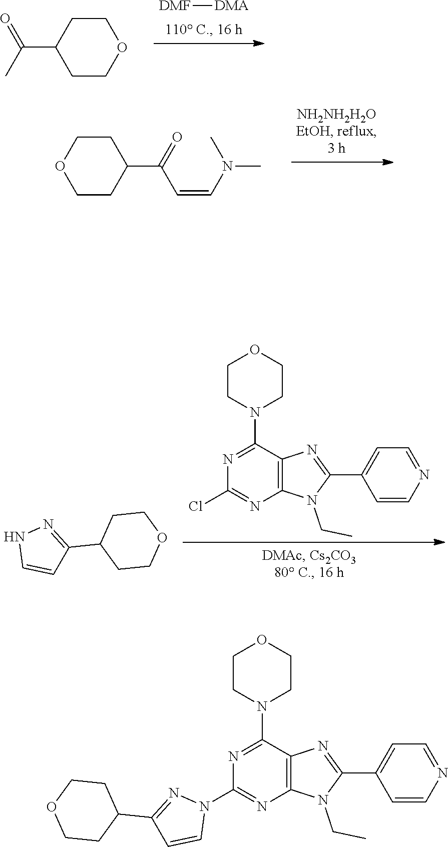

### Step 1: Preparation of (Z)-3-(dimethylamino)-1-(tetrahydro-2H-pyran-4-yl)prop-2-en-1-one

A solution of 1-(tetrahydro-2H-pyran-4-yl)ethan-1-one (1.28 g, 10 mmol) in N,N-dimethylformamide dimenthyl acetal (7.14 g, 60 mmol) was stirred at 110° C. for 16 h. The mixture was concentrated to give (Z)-3-(dimethylamino)-1-(tetrahydro-2H-pyran-4-yl)prop-2-en-1-one as a yellow solid. (1.41 g, 77%), which was used in the next step without further purification. LCMS (ESI) m/z: 184.2[M+H]+.

### Step 2: Preparation of 3-(tetrahydro-2H-pyran-4-yl)-1H-pyrazole

A mixture of (Z)-3-(dimethylamino)-1-(tetrahydro-2H-pyran-4-yl)prop-2-en-1-one (1.83 g, 1 mmol) and hydrazine hydrate (1.5 g, 3 mmol) in ethanol (10 mL) was stirred at 90° C. for 2 h. The reaction mixture was concentrated to give 3-(tetrahydro-2H-pyran-4-yl)-1H-pyrazole as red solid (1.04 g, 68%), which was used in the next step without further purification. LCMS (ESI) m/z: 153.3 [M+H]+.

### Step 3: 4-(9-ethyl-8-(pyridin-4-yl)-2-(3-(tetrahydro-2H-pyran-4-yl)-1H-pyrazol-1-yl)-9H-purin-6-yl)morpholine

To a mixture of 3-(tetrahydro-2H-pyran-4-yl)-1H-pyrazole (200 mg, 0.6 mmol) and cesium carbonate (390 mg, 1.2 mmol) in N,N-dimethylacetamide (10 mL) was added 4-(2-chloro-9-ethyl-8-(pyridin-4-yl)-9H-purin-6-yl)morpholine (83 mg, 0.66 mmol) portion wise, and the resulting mixture was stirred at 140° C. for 3 h. The mixture was concentrated and purified by prep-HPLC (SunFire C18, 4.6*50 mm, 3.5 um column Xbridge C18 3.5 μm 4.6×50 mm column. The mobile phase was acetonitrile/10 mM Formic acid aqueous solution.) to give the product as yellow solid (0.056 g, 27%).

¹H NMR (400 MHz, DMSO-d₆) δ 8.79 (d, J=5.4 Hz, 2H), 8.60 (d, J=2.4 Hz, 1H), 7.84 (d, J=5.6 Hz, 2H), 6.45 (d, J=2.4 Hz, 1H), 4.41 (dd, J=14.1, 7.0 Hz, 6H), 3.94 (d, J=8.3 Hz, 2H), 3.77 (s, 4H), 3.46 (t, J=10.9 Hz, 2H), 3.05-2.90 (m, 1H), 1.84 (d, J=12.6 Hz, 2H), 1.71 (qd, J=12.3, 4.2 Hz, 2H), 1.32 (t, J=7.1 Hz, 3H); LCMS (ESI) m/z: 460.8 [M+H]+.

### Synthesis of 4-(2-(3-(1,4-dioxan-2-yl)-1H-pyrazol-1-yl)-9-methyl-8-(pyridin-4-yl)-9H-purin-6-yl)morpholine (Compound 183)

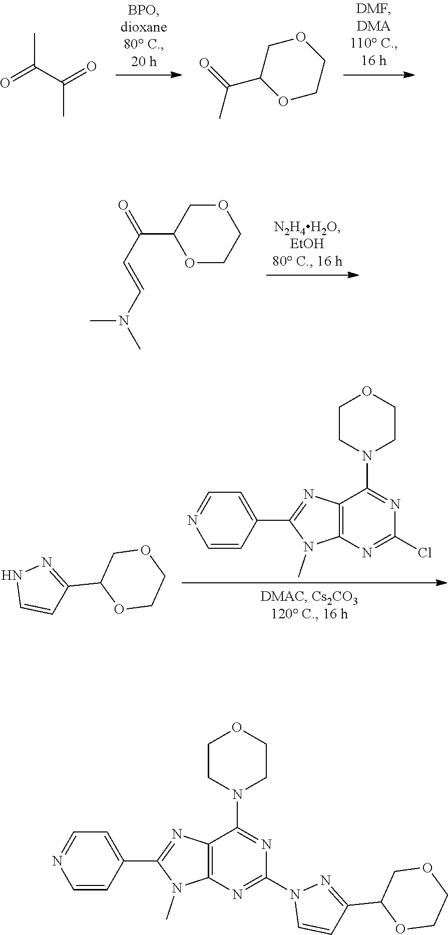

### Step 1: Synthesis of (1-(1,4-dioxan-2-yl)ethenone

To a mixture of biacetyl (860 mg, 10 mmol) in dioxane (20 mL) was added benzoyl peroxide (2.42 g, 10 mmol). The mixture was stirred at 80° C. for 16 h and water (20 mL) was added. The mixture was extracted with ethyl acetate (30 mL*3) and washed with aq. sodium hydroxide (10 mL*2). The organic layer was dried and concentrated to give (1-(1,4-dioxan-2-yl)ethanone as an yellow oil. (1.3 g, 99%). 1H NMR (400 MHz, CDCl3) δ 4.07 (dd, J=9.7, 3.2 Hz, 1H), 4.00 (dd, J=11.6, 3.2 Hz, 1H), 3.92-3.85 (m, 2H), 3.79 (dd, J=10.7, 2.7 Hz, 1H), 3.73 (dd, J=9.3, 2.1 Hz, 1H), 3.47 (dd, J=11.6, 9.7 Hz, 1H), 2.22 (s, 3H).

### Step 2: Synthesis of (E)-3-(dimethylamino)-1-(1,4-dioxan-2-yl)prop-2-en-1-one

A solution of (1-(1,4-dioxan-2-yl)ethanone (1.3 g, 1 mmol) in N,N-dimethylformamide dimethyl acetal (10 mL) was stirred at 110° C. for 16 h. The mixture was concentrated to give (E)-3-(dimethylamino)-1-(1,4-dioxan-2-yl)prop-2-en-1-one as an yellow oil. (1.85 g, 99%). LCMS: m/z=186.1 (M+H)+.

### Step 3: Synthesis of 3-(1,4-dioxan-2-yl)-1H-pyrazole

A mixture of (E)-3-(dimethylamino)-1-(1,4-dioxan-2-yl)prop-2-en-1-one (1.85 g, 10 mmol) and NH2NH2·H2O (1.5 g, 30 mol) in EtOH (20 mL) was stirred at 80° C. for 16 h. The reaction mixture was concentrated and purified by pre-HPLC (BOSTON pHlex ODS 10 μm 21.2×250 mm 120 A. The mobile phase was acetonitrile/0.1% Ammonium bicarbonate) to give 3-(1,4-dioxan-2-yl)-1H-pyrazole as an yellow oil. (250 mg, 16.2%). LCMS: [M+H]+=155.2.

### Step 4: Synthesis of 4-(2-(3-(1,4-dioxan-2-yl)-1H-pyrazol-1-yl)-9-methyl-8-(pyridin-4-yl)-9H-purin-6-yl)morpholine

A mixture of 4-(2-chloro-9-methyl-8-(pyridin-4-yl)-9H-purin-6-yl)morpholine (66 mg, 0.2 mmol), 3-(1,4-dioxan-2-yl)-1H-pyrazole (31 mg, 0.2 mmol) and cesium carbonate (196 mg, 0.6 mmol) in N,N-dimethylformamide (3 mL) was stirred at 120° C. for 16 h. The mixture was purified by pre-HPLC (BOSTON pHlex ODS 10 μm 21.2×250 mm 120 A. The mobile phase was acetonitrile/0.1% Ammonium bicarbonate) to afford the product 4-(2-(3-(1,4-dioxan-2-yl)-1H-pyrazol-1-yl)-9-methyl-8-(pyridin-4-yl)-9H-purin-6-yl)morpholine (72.5 mg, 54%) as a white solid. 1H NMR (500 MHz, DMSO-d₆) δ 8.78 (d, J=5.9 Hz, 2H), 8.67 (d, J=2.6 Hz, 1H), 7.90 (dd, J=4.6, 1.5 Hz, 2H), 6.53 (d, J=2.6 Hz, 1H), 4.71 (dd, J=10.0, 2.8 Hz, 1H), 4.35 (s, 4H), 3.93 (s, 3H), 3.92-3.89 (m, 1H), 3.87-3.83 (m, 1H), 3.81-3.72 (m, 6H), 3.70-3.55 (m, 2H); LCMS: [M+H]+=449.1.

The following compound was synthesized according to the protocol described above:

| Name | Structure | NMR, MS | # |
|---|---|---|---|
| 2-(1-(9-methyl-6- morpholino-8- (pyridin-4-yl)-9H- purin-2-yl)-1H- pyrazol-3- yl)morpholine | 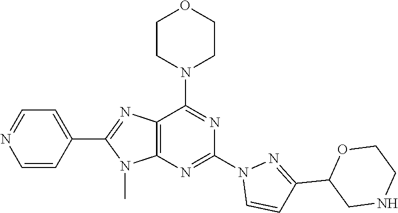 [SMILES: Cn1c(-c2ccncc2)nc2c(N3CCOCC3)nc(-n3ccc(C4CNCCO4)n3)nc21] | ¹H NMR (400 MHz, DMSO-d₆) δ 8.78 (dd, J = 4.8, 1.6 Hz, 2H), 8.63 (d, J = 2.6 Hz, 1H), 7.91 (dd, J = 4.8, 1.6Hz, 2H), 6.48 (d, J = 2.6 Hz, 1H), 4.52 (dd, J = 10.2, 2.4 Hz, 1H), 4.33 (s, 4H), 3.94 (s, 3H), 3.83 (d, J = 10.8 Hz, 1H), 3.81-3.74 (m, 4H), 3.65-3.55 (m, 1H), 2.99 (dd, J = 12.4, 2.4 Hz, 1H), 2.77 (dd, J = 18.2, 7.6 Hz, 3H); LCMS: [M + H]⁺ = 448.2. | 184 |

### Synthesis of 4-(9-ethyl-8-(pyridin-4-yl)-2-(3-(tetrahydro-2H-pyran-3-yl)-1H-pyrazol-1-yl)-9H-purin-6-yl)morpholine (Compound 185)

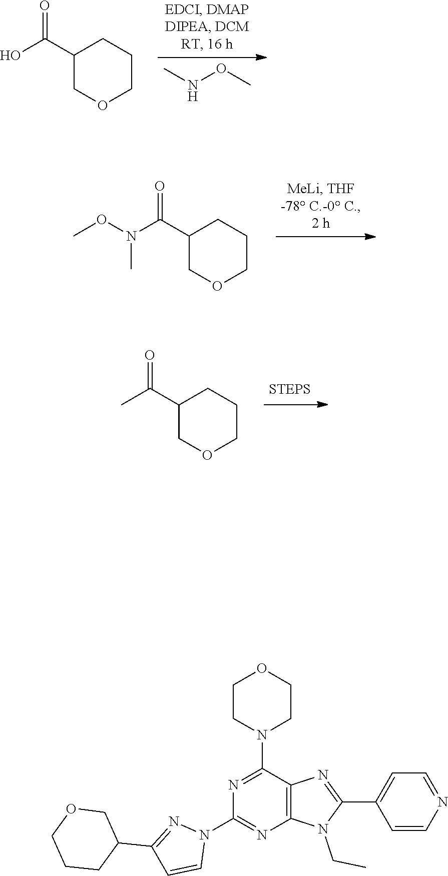

### Step 1: Preparation of tetrahydro-2H-pyran-3-carboxylic acid

To a solution of N,O-dimethylhydroxylamine hydrochloride (1.46 g, 15 mmol) in DCM (10 mL) were added tetrahydro-2H-pyran-3-carboxylic acid (1.30 g, 10 mmol), N-(3-dimethylaminopropyl)-N′-ethylcarbodiimide hydrochloride (2.87 g, 15 mmol), N,N-diisopropylethylamine (2.58 g, 20 mmol) and 4-dimethylaminepyridine (0.123 g, 1 mmol) at 25° C. The reaction mixture was stirred at room temperature for 16 h. It was diluted with water (20 mL) and extracted with dichloromethane (20 mL*2). The organic layer was dried and concentrated to give N-methoxy-N-methyltetrahydro-2H-pyran-3-carboxamide as a yellow oil. (1.73 g, 99%). LCMS (ESI) m/z: 174.1 [M+H]+.

### Step 2: Preparation of 1-(tetrahydro-2H-pyran-3-yl)ethan-1-one

To a solution of N-methoxy-N-methyltetrahydro-2H-pyran-3-carboxamide (1.73 g, 10 mmol) in dry tetrahydrofuran (10 mL) was added methyllithium (1.6M, 10 mmol, 6.25 mL) at −78° C. and the reaction was stirred at 0° C. for 1.5 h. The mixture was quenched with hydrochloric acid (0.5 M, 8.3 mL) and extracted with ethyl acetate (20 mL*2). The combined organic layer was washed with water (10 mL), dried and concentrated to give 1-(tetrahydro-2H-pyran-3-yl)ethan-1-one as yellow oil. (0.911 g, 71.2%). It would be used directly at next step.

The remaining steps were performed similar to the protocol described for the compound 183 to obtain, 4-(9-ethyl-8-(pyridin-4-yl)-2-(3-(tetrahydro-2H-pyran-3-yl)-1H-pyrazol-1-yl)-9H-purin-6-yl)morpholine

¹H NMR (400 MHz, DMSO-d₆) δ 8.79 (d, J=5.8 Hz, 2H), 8.60 (d, J=2.6 Hz, 1H), 7.84 (d, J=5.9 Hz, 2H), 6.45 (d, J=2.6 Hz, 1H), 4.74-4.06 (m, 6H), 3.97 (dd, J=10.9, 3.2 Hz, 1H), 3.86 (d, J=11.4 Hz, 1H), 3.82-3.72 (m, 4H), 3.41 (dt, J=12.3, 8.8 Hz, 2H), 3.02-2.90 (m, 1H), 2.06 (d, J=12.8 Hz, 1H), 1.80-1.61 (m, 3H), 1.32 (t, J=7.2 Hz, 3H); LCMS (ESI) m/z: 460.08.2[M+H]+.

### Synthesis of 4-(9-methyl-2-(3-phenyl-1H-pyrazol-1-yl)-8-(pyrrolidin-3-yl)-9H-purin-6-yl)morpholine (Compound 186)

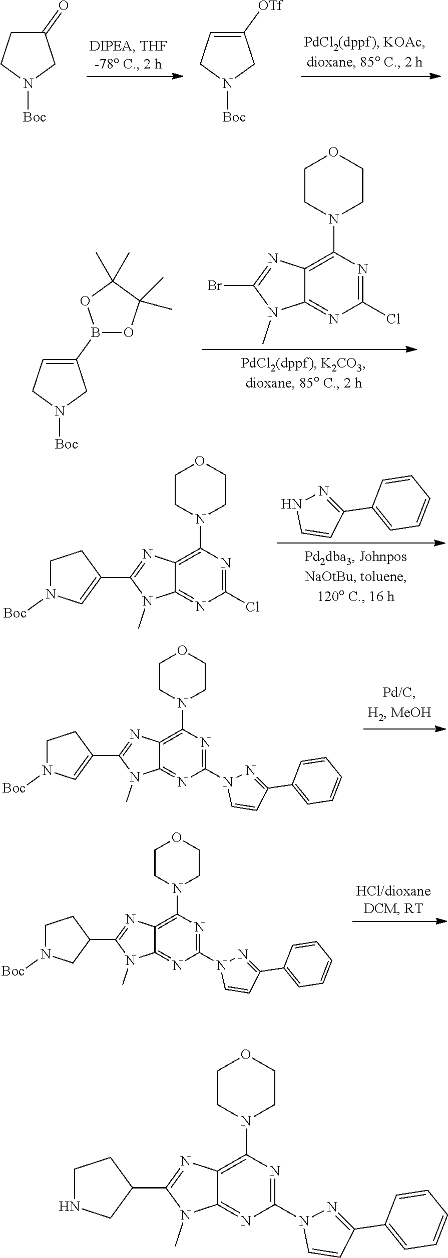

### Step 1: Synthesis of tert-butyl 3-(((trifluoromethyl)sulfonyl)oxy)-2,5-dihydro-1H-pyrrole-1-carboxylate

A solution of tert-butyl 3-oxopyrrolidine-1-carboxylate (1400 mg, 7.567 mmol) and N,N-diisopropylethylamine (2928 mg, 22.701 mmol) in dichloromethane (50 mL) was cooled to −78° C. for 10 mins. Then trifluoromethanesulfonic anhydride (2560 mg, 9.081 mmol) was added. The mixture was warmed up and stirred at 25° C. for 16 h. Then, ammonium chloride (aq) was added and the mixture was extracted with dichloromethane (50 mL×3). The organic layer was dried and concentrated to give tert-butyl 3-(((trifluoromethyl)sulfonyl)oxy)-2,5-dihydro-1H-pyrrole-1-carboxylate (800 mg, 33%) as a yellow oil. LC-MS: m/z=262 (M−56+H)⁺.

### Step 2: Synthesis of tert-Butyl 3-(4,4,5,5-tetramethyl-1,3,2-dioxaborolan-2-yl)-2,5-dihydro-1H-pyrrole-1-carboxylate

A mixture of tert-butyl 3-(((trifluoromethyl)sulfonyl)oxy)-2,5-dihydro-1H-pyrrole-1-carboxylate (2800 mg, 8.832 mmol), 4,4,4′,4′,5,5,5′,5′-octamethyl-2,2′-bi(1,3,2-dioxaborolane) (4487 mg, 17.665 mmol), [1,1′-bis(diphenylphosphino)ferrocene]dichloropalladium(II) (325 mg, 0.441 mmol) and potassium acetate (2600 mg, 26.532 mmol) in dioxane (80 mL) was stirred at 75° C. for 4 h. Then water was added and the mixture was extracted with ethyl acetate (50 mL×3). The organic layer was dried and concentrated. The crude product was purified by silica gel column (petroleum ether:ethyl acetate from 50:1 to 10:1) to give tert-butyl 3-(4,4,5,5-tetramethyl-1,3,2-dioxaborolan-2-yl)-2,5-dihydro-1H-pyrrole-1-carboxylate (2050 mg, 78%) as a yellow solid. LC-MS: m/z=240 (M−56+H)⁺.

### Step 3: Synthesis of tert-Butyl 4-(2-chloro-9-methyl-6-morpholino-9H-purin-8-yl)-2,3-dihydro-1H-pyrrole-1-carboxylate

A solution of tert-butyl 3-(4,4,5,5-tetramethyl-1,3,2-dioxaborolan-2-yl)-2,5-dihydro-1H-pyrrole-1-carboxylate (307 mg, 1.042 mmol), 4-(8-bromo-2-chloro-9-methyl-9H-purin-6-yl)morpholine (230 mg, 0.695 mmol), bis(diphenylphosphino)ferrocene]dichloropalladium(II) (25 mg, 0.0347 mmol) and potassium carbonate (287 mg, 2.085 mmol) in dioxane/water (8 mL) was stirred at 85° C. for 4 h and concentrated. The crude product thus obtained was purified by silica gel column (petroleum ether:ethyl acetate from 50:1 to 10:1) to obtain tert-butyl 4-(2-chloro-9-methyl-6-morpholino-9H-purin-8-yl)-2,3-dihydro-1H-pyrrole-1-carboxylate (150 mg, 51%) as a yellow solid. LC-MS: m/z=421 (M+H)⁺.

### Step 4: Synthesis of tert-Butyl 4-(9-methyl-6-morpholino-2-(3-phenyl-1H-pyrazol-1-yl)-9H-purin-8-yl)-2,3-dihydro-1H-pyrrole-1-carboxylate

A mixture of tert-butyl 4-(2-chloro-9-methyl-6-morpholino-9H-purin-8-yl)-2,3-dihydro-1H-pyrrole-1-carboxylate (110 mg, 0.262 mmol), 3-phenyl-1H-pyrazole (41 mg, 0.288 mmol), tris(dibenzylideneacetone)dipalladium (23 mg, 0.052 mmol), Johnphos (16 mg, 0.052 mmol) and sodium tert-butoxide (75 mg, 0.786 mmol) in toluene (3 mL) was stirred at 120° C. for 16 h. Water was added and the mixture was extracted ethyl acetate. The organic layer was dried and concentrated. The crude product was purified by silica gel column (petroleum ether:ethyl acetate from 50:1 to 10:1) to give tert-butyl 4-(9-methyl-6-morpholino-2-(3-phenyl-1H-pyrazol-1-yl)-9H-purin-8-yl)-2,3-dihydro-1H-pyrrole-1-carboxylate (70 mg, 51%) as a yellow solid. LC-MS: m/z=529 (M+H)⁺.

### Step 5: Synthesis of tert-Butyl 3-(9-methyl-6-morpholino-2-(3-phenyl-1H-pyrazol-1-yl)-9H-purin-8-yl)pyrrolidine-1-carboxylate

A suspension of tert-butyl 4-(9-methyl-6-morpholino-2-(3-phenyl-1H-pyrazol-1-yl)-9H-purin-8-yl)-2,3-dihydro-1H-pyrrole-1-carboxylate (70 mg, 0.132 mmol) and Pd/C (10%, 40 mg) in methanol (4 mL) was stirred at 25° C. for 16 h under hydrogen atmosphere. The mixture was filtered and the filtrate was concentrated to obtain tert-butyl 3-(9-methyl-6-morpholino-2-(3-phenyl-1H-pyrazol-1-yl)-9H-purin-8-yl)pyrrolidine-1-carboxylate (50 mg, 71%) as a yellow solid. LC-MS: m/z=531 (M+H)⁺.

### Step 6: Synthesis of 4-(9-Methyl-2-(3-phenyl-1H-pyrazol-1-yl)-8-(pyrrolidin-3-yl)-9H-purin-6-yl)morpholine

A solution of tert-butyl 3-(9-methyl-6-morpholino-2-(3-phenyl-1H-pyrazol-1-yl)-9H-purin-8-yl)pyrrolidine-1-carboxylate (50 mg, 0.094 mmol), and hydrogen chloride/dioxane (2 mL) in dichloromethane (4 mL) was stirred at 25° C. for 2 h. The mixture was concentrated and the crude was purified by prep-HPLC(Column Xbridge 21.2*250 mm C18, 10 um, mobile phase A: water (10 mmol/L ammonium bicarbonate) B: acetonitrile) to obtain 4-(9-Methyl-2-(3-phenyl-1H-pyrazol-1-yl)-8-(pyrrolidin-3-yl)-9H-purin-6-yl)morpholine (8.5 mg, 21%) as white solid.

¹H NMR (400 MHz, DMSO-d₆) δ 8.72 (d, J=2.4 Hz, 1H), 7.96 (d, J=7.2 Hz, 2H), 7.47 (t, J=7.6 Hz, 2H), 7.39-7.36 (m, 1H), 7.03 (d, J=2.0 Hz, 1H), 4.28-4.12 (m, 4H), 3.77-3.75 (m, 7H), 3.56-3.54 (m, 1H), 3.27-3.24 (m, 1H), 3.08 (s, 1H), 2.98-2.93 (m, 2H), 2.16-2.06 (m, 3H); LC-MS: m/z=431 (M+H)⁺.

### Synthesis of 4-(8-(3,6-dihydro-2H-pyran-4-yl)-9-methyl-2-(3-(1-methylpyrrolidin-3-yl)-1H-pyrazol-1-yl)-9H-purin-6-yl)morpholine (Compound 187)

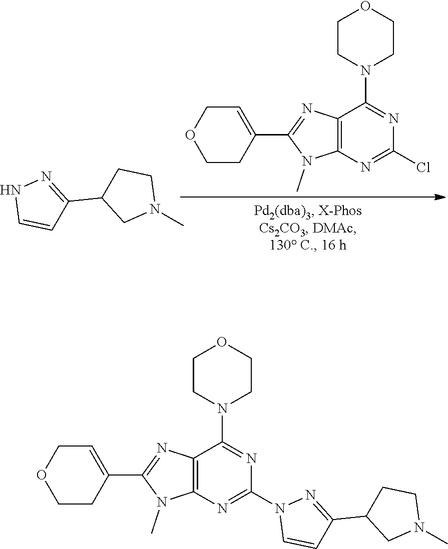 [SMILES: CN1CCC(c2cc[nH]n2)C1 ; Cn1c(C2=CCOCC2)nc2c(N3CCOCC3)nc(Cl)nc21 ; CN1CCC(c2ccn(-c3nc(N4CCOCC4)c4nc(C5=CCOCC5)n(C)c4n3)n2)C1]

To a solution of 4-(2-chloro-8-(3,6-dihydro-2H-pyran-4-yl)-9-methyl-9H-purin-6-yl)morpholine (0.14 g, 0.42 mmol) in DMAc (4 mL) were added 3-(1-methylpyrrolidin-3-yl)-1H-pyrazole (0.082 g, 0.54 mmol), cesium carbonate (0.204 g, 0.63 mmol), 2-(dicyclohexylphosphino)-2′,4′,6′-triisopropylbiphenyl (0.038 g, 0.08 mmol) and tris(dibenzylideneacetone) dipalladium(0) (0.037 g, 0.04 mmol) at 25° C. and the resultant reaction mixture was stirred at 130° C. for 16 h under argon. The mixture was filtered and purified by Prep-HPLC(SunFire C18, 4.6*50 mm, 3.5 um column Xbridge C18 3.5 μm 4.6×50 mm column. The mobile phase was acetonitrile/10 mM ammonium bicarbonate aqueous solution.) to obtain the desired product (13.4 mg, 7%) as off-white solid.

¹H NMR (400 MHz, DMSO-d₆) δ 8.54 (d, J=2.5 Hz, 1H), 6.55 (dd, J=6.2, 2.0 Hz, 1H), 6.38 (d, J=2.5 Hz, 1H), 4.86 (dd, J=6.2, 3.2 Hz, 1H), 4.50-4.20 (m, 5H), 4.15-4.05 (m, 1H), 3.90-3.80 (m, 1H), 3.79-3.55 (m, 7H), 3.49-3.45 (m, 1H), 2.91 (t, J=8.4 Hz, 1H), 2.70-2.55 (m, 2H), 2.52 (d, J=9.5 Hz, 1H), 2.31 (s, 3H), 2.25-2.09 (m, 3H), 1.90-1.80 (m, 1H); LCMS (ESI) m/z: 451.2 [M+H]⁺.

### Synthesis of tert-butyl 4-(1-(6-morpholino-8-(pyridin-4-yl)-9H-purin-2-yl)-1H-pyrazol-3-yl)piperidine-1-carboxylate (Compound 188), 4-(2-(3-(piperidin-4-yl)-1H-pyrazol-1-yl)-8-(pyridin-4-yl)-9H-purin-6-yl)morpholine (Compound 189) and 2-methyl-1-(4-(1-(6-morpholino-8-(pyridin-4-yl)-9H-purin-2-yl)-1H-pyrazol-3-yl)piperidin-1-yl)propan-1-one (Compound 190)

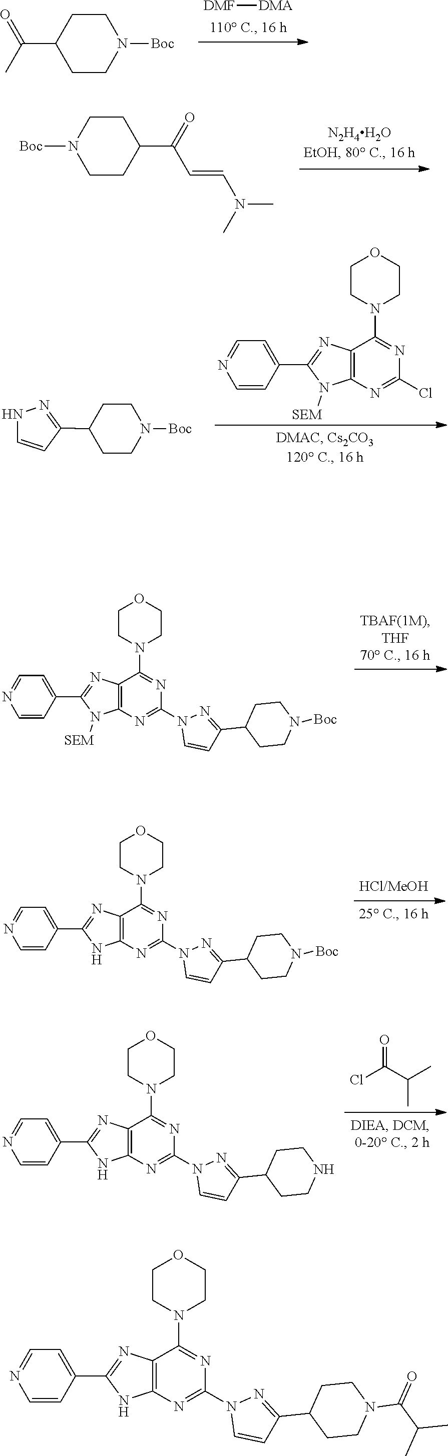

### Step 1: Synthesis of (E)-tert-butyl 4-(3-(dimethylamino)acryloyl)piperidine-1-carboxylate

A solution of tert-butyl 4-acetylpiperidine-1-carboxylate (2.273 g, 10 mmol) in N,N-Dimethylformamide dimethyl acetal (20 mL) was stirred at 110° C. for 16 h. The mixture was concentrated to give (E)-tert-butyl 4-(3-(dimethylamino)acryloyl)piperidine-1-carboxylate as a yellow solid. (3 g, crude). LCMS, [M+H]+=283.2.

### Step 2: Synthesis of tert-butyl 4-(1H-pyrazol-3-yl)piperidine-1-carboxylate

A mixture of (E)-tert-butyl 4-(3-(dimethylamino)acryloyl)piperidine-1-carboxylate (2.72 g, 9.65 mmol) and hydrazine hydrate (1.45 g, 28.94 mol) in ethanol (20 mL) was stirred at 80° C. for 16 h. The reaction mixture was concentrated and purified by pre-HPLC (BOSTON pHlex ODS 10 μm 21.2×250 mm 120 A. The mobile phase was acetonitrile/0.1% Ammonium bicarbonate) to give tert-butyl 4-(1H-pyrazol-3-yl)piperidine-1-carboxylate as a yellow oil. (1.2 g, 48%). LCMS, [M−56+H]+=196.2.

### Step 3: Synthesis of tert-butyl 4-(1-(6-morpholino-8-(pyridin-4-yl)-9-((2-(trimethylsilyl)ethoxy)methyl)-9H-purin-2-yl)-1H-pyrazol-3-yl)piperidine-1-carboxylate

A mixture 4-(2-chloro-8-(pyridin-4-yl)-9-((2-(trimethylsilyl)ethoxy)methyl)-9H-purin-6-yl)morpholine (445 mg, 1 mmol) and tert-butyl 4-(1H-pyrazol-3-yl)piperidine-1-carboxylate (250 mg, 1 mmol) and cesium carbonate (978 mg, 3 mmol) in N,N-dimethylformamide (20 mL) was stirred at 120° C. for 16 h. The mixture was poured into water (100 mL), extracted with ethyl acetate (200 mL*2) and washed with water (50 mL*3). The organic layer was dried and concentrated to give crude product tert-butyl 4-(1-(6-morpholino-8-(pyridin-4-yl)-9-((2-(trimethylsilyl)ethoxy)methyl)-9H-purin-2-yl)-1H-pyrazol-3-yl)piperidine-1-carboxylate as a yellow solid (650 mg, crude. LCMSA, [M+H]+=662.3.

### Step 4: Synthesis of tert-butyl 4-(1-(6-morpholino-8-(pyridin-4-yl)-9H-purin-2-yl)-1H-pyrazol-3-yl)piperidine-1-carboxylate

A mixture of tert-butyl 4-(1-(6-morpholino-8-(pyridin-4-yl)-9-((2-(trimethylsilyl)ethoxy)methyl)-9H-purin-2-yl)-1H-pyrazol-3-yl)piperidine-1-carboxylate (180 mg, 0.27 mmol) and tetrabutylammonium fluoride (2 mL, 1 M in THF) in tetrahydrofuran (5 mL) was stirred at 70° C. for 16 h. The mixture was concentrated and the crude product was purified by pre-HPLC (BOSTON pHlex ODS 10 μm 21.2×250 mm 120 A. The mobile phase was acetonitrile/0.1% Ammonium bicarbonate) to afford tert-butyl 4-(1-(6-morpholino-8-(pyridin-4-yl)-9H-purin-2-yl)-1H-pyrazol-3-yl)piperidine-1-carboxylate (104 mg, 52%) as a white solid. 1H NMR (400 MHz, DMSO-d₆) δ 14.12 (s, 1H), 8.74 (s, 2H), 8.54 (d, J=2.6 Hz, 1H), 8.01 (d, J=5.4 Hz, 2H), 6.44 (d, J=2.6 Hz, 1H), 4.36 (s, 4H), 4.00 (d, J=11.1 Hz, 2H), 3.85-3.75 (m, 4H), 2.87 (dd, J=13.2, 9.6 Hz, 3H), 1.94 (dd, J=13.1, 2.3 Hz, 2H), 1.53 (qd, J=12.5, 3.9 Hz, 2H), 1.42 (s, 9H); LCMSA011 [M+H]+=531.9.

### Step 5: Synthesis of 4-(2-(3-(piperidin-4-yl)-1H-pyrazol-1-yl)-8-(pyridin-4-yl)-9H-purin-6-yl)morpholine

A mixture of tert-butyl 4-(1-(6-morpholino-8-(pyridin-4-yl)-9H-purin-2-yl)-1H-pyrazol-3-yl)piperidine-1-carboxylate (120 mg, 0.226 mmol) in hydrochloric acid (3M in methanol, 2 mL) was stirred at 25° C. for 16 h. The mixture was concentrated to get crude product which was purified by pre-HPLC (BOSTON pHlex ODS 10 μm 21.2×250 mm 120 A. The mobile phase was acetonitrile/0.1% Ammonium bicarbonate) to afford 4-(2-(3-(piperidin-4-yl)-1H-pyrazol-1-yl)-8-(pyridin-4-yl)-9H-purin-6-yl)morpholine (50 mg, 27%) as a white solid.

1H NMR (400 MHz, DMSO-d₆) δ 8.63 (d, J=6.0 Hz, 2H), 8.52 (d, J=2.5 Hz, 1H), 8.02 (dd, J=4.5, 1.6 Hz, 2H), 6.37 (d, J=2.5 Hz, 1H), 4.33 (s, 4H), 3.81-3.75 (m, 4H), 3.27 (d, J=11.9 Hz, 2H), 3.09-2.94 (m, 3H), 2.03 (d, J=12.7 Hz, 2H), 1.85-1.68 (m, 2H); LCMS, [M+H]+=432.1.

### Step 6: Synthesis of 2-methyl-1-(4-(1-(6-morpholino-8-(pyridin-4-yl)-9H-purin-2-yl)-1H-pyrazol-3-yl)piperidin-1-yl)propan-1-one

To a solution of 4-(2-(3-(piperidin-4-yl)-1H-pyrazol-1-yl)-8-(pyridin-4-yl)-9H-purin-6-yl)morpholine (100 mg, 0.23 mmol) and N,N-Diisopropylethylamine (89 mg, 0.69 mmol) in dry dichloromethane (5 mL) was added isobutyryl chloride (30 mg, 0.28 mmol) drop wise at 0° C. and the resultant mixture was stirred at 20° C. for 2 h. The mixture was concentrated and the resultant crude product was purified by pre-HPLC (BOSTON pHlex ODS 10 μm 21.2×250 mm 120 A. The mobile phase was acetonitrile/0.1% Ammonium bicarbonate) to obtain 2-methyl-1-(4-(1-(6-morpholino-8-(pyridin-4-yl)-9H-purin-2-yl)-1H-pyrazol-3-yl)piperidin-1-yl)propan-1-one (28.4 mg, 20%) as white solid.

1H NMR (400 MHz, DMSO-d₆) δ 14.11 (s, 1H), 8.73 (d, J=6.0 Hz, 2H), 8.54 (d, J=2.6 Hz, 1H), 8.01 (d, J=6.1 Hz, 2H), 6.44 (d, J=2.5 Hz, 1H), 4.43 (d, J=11.7 Hz, 5H), 4.01 (d, J=12.5 Hz, 1H), 3.90-3.72 (m, 4H), 3.19 (t, J=11.8 Hz, 1H), 3.00-2.86 (m, 2H), 2.75-2.60 (m, 1H), 2.07-1.92 (m, 2H), 1.60-1.40 (m, 2H), 1.02 (d, J=3.7 Hz, 6H); LCMS, [M+H]+=501.9.

The following compounds were synthesized according to the protocol described above:

| Name | Structure | NMR, MS | # |
|---|---|---|---|
| tert-butyl 3-(1-(9- methyl-6- morpholino-8- (pyridin-2-yl)-9H- purin-2-yl)-1H- pyrazol-3- yl)pyrrolidine-1- carboxylate | 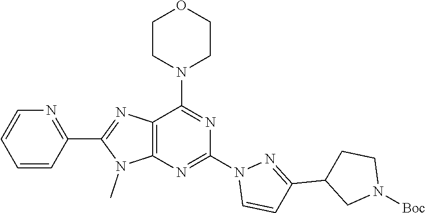 [SMILES: Cn1c(-c2ccccn2)nc2c(N3CCOCC3)nc(-n3ccc(C4CCN(C(=O)OC(C)(C)C)C4)n3)nc21] | 1H NMR (400 MHz, CDCI3) δ 8.68 (d, J = 4.1 Hz, 1H), 8.52 (d, J = 2.7 Hz, 1H), 8.28 (d, J = 8.1 Hz, 1H), 7.82 (dd, J = 8.7, 7.0 Hz, 1H), 7.38-7.29 (m, 2H), 6.31 (d, J = 2.6 Hz, 1H), 4.31 (s, 4H), 4.31 (s, 3H), 3.93-3.88 (m, 4H), 3.84 (s, 1H), 3.85-3.70 (m, 4H), 2.21 (d, J = 91.2 Hz, 2H), 1.48 (s, 9H); LCMS (ESI) m/z: 532.7 [M + H]⁺. | 191 |
| 4-(9-methyl-8- (pyridin-2-yl)-2- (3-(pyrrolidin-3- yl)-1H-pyrazol-1- yl)-9H-purin-6- yl)morpholine | 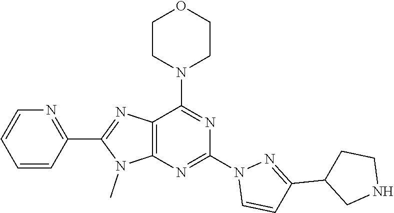 [SMILES: Cn1c(-c2ccccn2)nc2c(N3CCOCC3)nc(-n3ccc(C4CCNC4)n3)nc21] | 1H NMR (400 MHz, CDCI3) δ 8.69 (s, 1H), 8.51 (s, 1H), 8.27 (d, J = 7.9 Hz, 1H), 7.83 (d, J = 8.7 Hz, 1H), 7.33 (d, J = 4.8 Hz, 1H), 6.32 (d, J = 2.6 Hz, 1H), 4.30 (s, 4H), 4.30 (d, J = 7.3 Hz, 3H), 3.90 (s, 4H), 3.61 (d, J = 7.5 Hz, 1H), 3.38 (d, J = 89.6 Hz, 4H), 2.21 (d, J = 111.7 Hz, 2H); LCMS (ESI) m/z: 432.2 [M + H]⁺ | 192 |
| 1-(3-(1-(9- methyl-6- morpholino-8- (pyridin-2-yl)-9H- purin-2-yl)-1H- pyrazol-3- yl)pyrrolidin-1- yl)ethan-1-one | 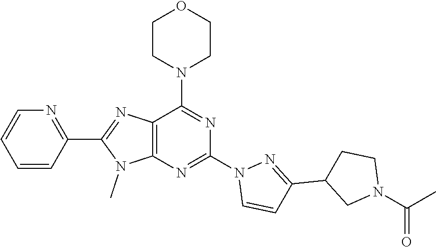 [SMILES: CC(=O)N1CCC(c2ccn(-c3nc(N4CCOCC4)c4nc(-c5ccccn5)n(C)c4n3)n2)C1] | 1H NMR (400 MHz, DMSO-d₆) δ 8.74 (d, J = 4.3 Hz, 1H), 8.64 (dd, J = 5.8, 2.6 Hz, 1H), 8.26 (d, J = 8.0 Hz, 1H), 8.00 (td, J = 7.8, 1.8 Hz, 1H), 7.53 (dd, J = 6.6, 4.9 Hz, 1H), 6.50 (dd, J = 8.1, 2.6 Hz, 1H), 4.17 (s, 4H), 4.17 (s, 3H), 3.90 (s, 1H), 3.84-3.77 (m, 4H), 3.69-3.35 (m, 4H), 2.33 (s, 2H), 1.98 (d, J = 5.5 Hz, 3H); LCMS (ESI) m/z: 474.7 [M + H]⁺. | 193 |
| 1-(3-(1-(9- methyl-6- morpholino-8- (pyridin-4-yl)-9H- purin-2-yl)-1H- pyrazol-3- yl)pyrrolidin-1- yl)ethanone | 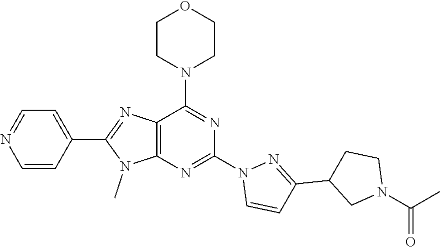 [SMILES: CC(=O)N1CCC(c2ccn(-c3nc(N4CCOCC4)c4nc(-c5ccncc5)n(C)c4n3)n2)C1] | 1H NMR (400 MHz, DMSO-d6) δ 8.78 (d, J = 5.9 Hz, 2H), 8.64 (dd, J = 5.7, 2.6 Hz, 1H), 7.90 (d, J = 6.0 Hz, 2H), 6.50 (dd, J = 8.1, 2.6 Hz, 1H), 4.33 (s, 4H), 3.92 (s, 3H), 3.89-3.75 (m, 5H), 3.67- 3.39 (m, 4H), 2.36-2.21 (m, 1H), 2.17-2.02 (m, 1H), 1.98 (d, J = 5.4 Hz, 3H). LCMS: [M + H]⁺ = 474.1. | 194 |

### Synthesis of tert-butyl 3-(9-methyl-6-morpholino-2-(3-phenyl-1H-pyrazol-1-yl)-9H-purin-8-yl)azetidine-1-carboxylate (Compound 195), 4-(8-(azetidin-3-yl)-9-methyl-2-(3-phenyl-1H-pyrazol-1-yl)-9H-purin-6-yl)morpholine (Compound 196) and 4-(9-methyl-8-(1-methylazetidin-3-yl)-2-(3-phenyl-1H-pyrazol-1-yl)-9H-purin-6-yl)morpholine (Compound 197)

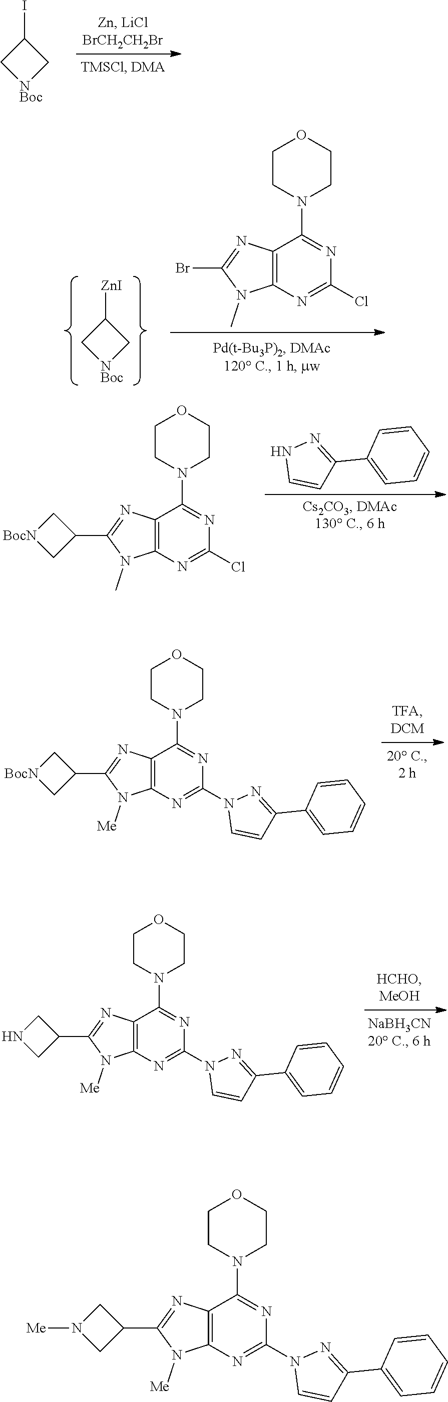

### Step 1: Preparation of tert-butyl 3-(2-chloro-9-methyl-6-morpholino-9H-purin-8-yl)azetidine-1-carboxylate

To a dry 2-necked flask lithium chloride (549 mg, 13.08 mmol) and activated zinc dust (854 mg, 13.06 mmol) were added followed by N,N-dimethylacetamide (8 mL). A solution of 1,2-dibromethane (0.2 mL) in N,N-dimethylacetamide (0.5 mL) was then added dropwise with stirring. A solution of trimethylsilyl chloride (0.1 mL) in N,N-dimethylacetamide (0.5 mL) was also added dropwise and the mixture was stirred for 30 min at 40° C. A solution of tert-butyl 3-iodoazetidine-1-carboxylate (1847 mg, 6.526 mmol) in N,N-dimethylacetamide (1 mL) was then added dropwise. The resulting mixture was stirred at 40° C. for 1 h and then cooled to 20° C. To the above solution was added dropwise a solution of 4-(8-bromo-2-chloro-9-methyl-9H-purin-6-yl)morpholine (720 mg, 2.175 mmol) and bis(tri-tert-butylphosphine)palladium(0) (111 mg, 0.218 mmol) in N,N-dimethylacetamide (4 mL). Then, the mixture was stirred at 120° C. under microwave for 1 h. The resultant crude product was purified by HPLC (SunFire C18, 4.6*50 mm, 3.5 um column Xbridge C18 3.5 μm 4.6×50 mm column. The elution system used was a gradient of 5%-95% over 1.5 min at 2 ml/min and the solvent was acetonitrile/0.01% aqueous NH₄HCO₃) to obtain tert-butyl 3-(2-chloro-9-methyl-6-morpholino-9H-purin-8-yl)azetidine-1-carboxylate (318 mg, 36%) as white solid. LCMS (ESI) m/z: 409.2 [M+H]+.

### Step 2: Preparation of tert-butyl 3-(9-methyl-6-morpholino-2-(3-phenyl-1H-pyrazol-1-yl)-9H-purin-8-yl)azetidine-1-carboxylate

To a solution of tert-butyl 3-(2-chloro-9-methyl-6-morpholino-9H-purin-8-yl)azetidine-1-carboxylate (318 mg, 0.779 mmol) and 3-phenyl-1H-pyrazole (135 mg, 0.935 mmol) in N,N-dimethylacetamide (6 mL) was added cesium carbonate (568 mg, 1.743 mmol). Then the mixture was heated to 130° C. and stirred for 6 h. The resultant crude product was purified by HPLC (SunFire C18, 4.6*50 mm, 3.5 μm column Xbridge C18 3.5 μm 4.6×50 mm column. The elution system used was a gradient of 5%-95% over 1.5 min at 2 ml/min and the solvent was acetonitrile/0.01% aqueous NH₄HCO₃) to obtain tert-butyl 3-(9-methyl-6-morpholino-2-(3-phenyl-1H-pyrazol-1-yl)-9H-purin-8-yl)azetidine-1-carboxylate (198.2 mg, 49%) as light yellow solid.

¹H NMR (400 MHz, DMSO) δ 8.73 (d, J=2.6 Hz, 1H), 7.96 (d, J=7.2 Hz, 2H), 7.47 (t, J=7.5 Hz, 2H), 7.38 (t, J=7.3 Hz, 1H), 7.04 (d, J=2.7 Hz, 1H), 4.84-4.05 (m, 9H), 3.92-3.72 (m, 4H), 3.64 (s, 3H), 1.40 (s, 9H); LCMS (ESI) m/z: 517.2 [M+H]+.

### Step 3: Preparation of 4-(8-(azetidin-3-yl)-9-methyl-2-(3-phenyl-1H-pyrazol-1-yl)-9H-purin-6-yl)morpholine

To a solution of tert-butyl 3-(9-methyl-6-morpholino-2-(3-phenyl-1H-pyrazol-1-yl)-9H-purin-8-yl)azetidine-1-carboxylate (187 mg, 0.362 mmol) in dichloromethane (2 mL) was added trifluoroacetic acid (2 mL). The resultant mixture was stirred at 20° C. for 2 h and concentrated. The resultant crude product was purified by HPLC (SunFire C18, 4.6*50 mm, 3.5 um column Xbridge C18 3.5 μm 4.6×50 mm column. The elution system used was a gradient of 5%-95% over 1.5 min at 2 ml/min and the solvent was acetonitrile/0.01% aqueous NH₄HCO₃) to obtain 4-(8-(azetidin-3-yl)-9-methyl-2-(3-phenyl-1H-pyrazol-1-yl)-9H-purin-6-yl)morpholine (120 mg, 80%) as white solid.

¹H NMR (400 MHz, DMSO) δ 8.72 (d, J=2.6 Hz, 1H), 7.96 (d, J=7.1 Hz, 2H), 7.47 (t, J=7.5 Hz, 2H), 7.38 (t, J=7.3 Hz, 1H), 7.03 (d, J=2.7 Hz, 1H), 4.62-4.06 (m, 6H), 3.94 (t, J=7.3 Hz, 2H), 3.77 (m, 6H), 3.63 (s, 3H); LCMS (ESI) m/z: 417.1 [M+H]+.

### Step 4: Preparation of 4-(9-methyl-8-(1-methylazetidin-3-yl)-2-(3-phenyl-1H-pyrazol-1-yl)-9H-purin-6-yl)morpholine

To a solution of 4-(8-(azetidin-3-yl)-9-methyl-2-(3-phenyl-1H-pyrazol-1-yl)-9H-purin-6-yl)morpholine (80 mg, 0.192 mmol) and 30% formalin (192 mg, 1.922 mmol) in methanol (6 mL) was added one drop of acetic acid. The resulting mixture was stirred at 20° C. for 1 h and sodium cyanoborohydride (42 mg, 0.672 mmol) was added. The mixture was stirred for another 1 h and concentrated. The crude product was then purified by HPLC (SunFire C18, 4.6*50 mm, 3.5 um column Xbridge C18 3.5 μm 4.6×50 mm column. The elution system used was a gradient of 5%-95% over 1.5 min at 2 ml/min and the solvent was acetonitrile/0.01% aqueous NH₄HCO₃) to obtain 4-(9-methyl-8-(1-methylazetidin-3-yl)-2-(3-phenyl-1H-pyrazol-1-yl)-9H-purin-6-yl)morpholine (29.5 mg, 36%) as white solid.

¹H NMR (400 MHz, DMSO) δ 8.72 (d, J=2.7 Hz, 1H), 8.04-7.91 (m, 2H), 7.47 (t, J=7.5 Hz, 2H), 7.38 (t, J=7.3 Hz, 1H), 7.03 (d, J=2.7 Hz, 1H), 4.31 (s, 4H), 3.95 (pent, J=8 Hz, 1H), 3.86-3.75 (m, 4H), 3.71 (t, J=7.3 Hz, 2H), 3.64 (s, 3H), 3.38 (t, J=7.1 Hz, 2H), 2.28 (s, 3H); LCMS (ESI) m/z: 431.2 [M+H]+.

### Synthesis of 4-(9-ethyl-2-(5-methyl-2H-indazol-2-yl)-8-(pyridin-4-yl)-9H-purin-6-yl)morpholine (Compound 198) and 4-(9-ethyl-2-(5-methyl-1H-indazol-1-yl)-8-(pyridin-4-yl)-9H-purin-6-yl)morpholine (Compound 199)

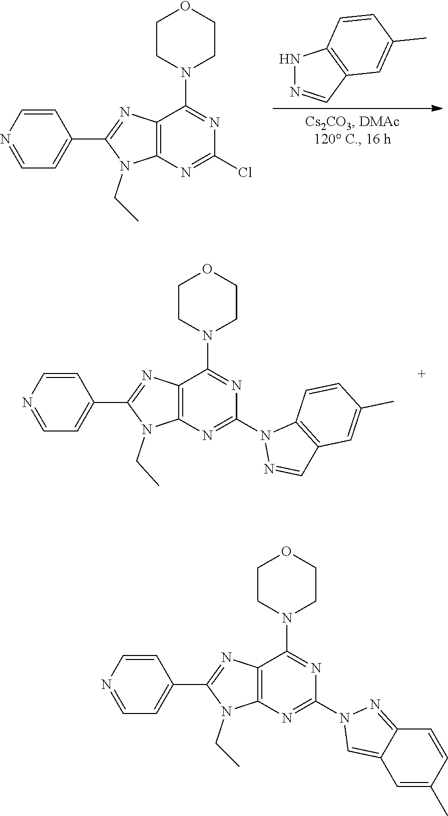

A mixture of 4-(2-chloro-9-ethyl-8-(pyridin-4-yl)-9H-purin-6-yl)morpholine (268.8 mg, 0.78 mmol), 5-methyl-1H-indazole (123.7 mg, 0.94 mmol) and cesium carbonate (763 mg, 2.34 mmol) in N,N-dimethylacetamide (5 mL) was stirred at 120° C. for 16 h. The mixture was filtered and purified by prep-HPLC (Column Xbridge 21.2*250 mm C18, 10 um, Mobile Phase A: water (10 mmol/L ammonium bicarbonate) B: acetonitrile) to afford 4-(9-ethyl-2-(5-methyl-1H-indazol-1-yl)-8-(pyridin-4-yl)-9H-purin-6-yl)morpholine (14.1 mg, 4.1%) and 4-(9-ethyl-2-(5-methyl-2H-indazol-2-yl)-8-(pyridin-4-yl)-9H-purin-6-yl)morpholine (18.2 mg, 5.3%) as white solids. They were confirmed by HMBC.

Compound 198: ¹H NMR (400 MHz, DMSO) δ 9.20 (s, 1H), 8.81 (d, J=6.0 Hz, 2H), 7.87 (dd, J=4.5, 1.6 Hz, 2H), 7.67 (d, J=9.0 Hz, 1H), 7.51 (s, 1H), 7.21-7.13 (m, 1H), 4.47 (q, J=7.1 Hz, 2H), 4.45-4.10 (m, 4H), 3.85-3.77 (m, 4H), 2.38 (s, 3H), 1.37 (t, J=7.2 Hz, 3H); LCMS (ESI) m/z: [M+H]+=441.1. Compound 199: ¹H NMR (400 MHz, DMSO) δ 8.81 (dd, J=4.5, 1.6 Hz, 2H), 8.56 (d, J=8.6 Hz, 1H), 8.32 (d, J=0.6 Hz, 1H), 7.85 (dd, J=4.5, 1.7 Hz, 2H), 7.66 (s, 1H), 7.42 (dd, J=8.8, 1.4 Hz, 1H), 4.46 (q, J=7.1 Hz, 6H), 3.87-3.75 (m, 4H), 2.46 (s, 3H), 1.41 (t, J=7.2 Hz, 3H); LCMS (ESI) m/z: [M+H]+=441.1.

### Synthesis of [1-[9-ethyl-6-morpholino-8-(4-pyridyl)purin-2-yl]indazol-5-yl]methanol and (2-(9-ethyl-6-morpholino-8-(pyridin-4-yl)-9H-purin-2-yl)-2H-indazol-5-yl)methanol (Compound 200)

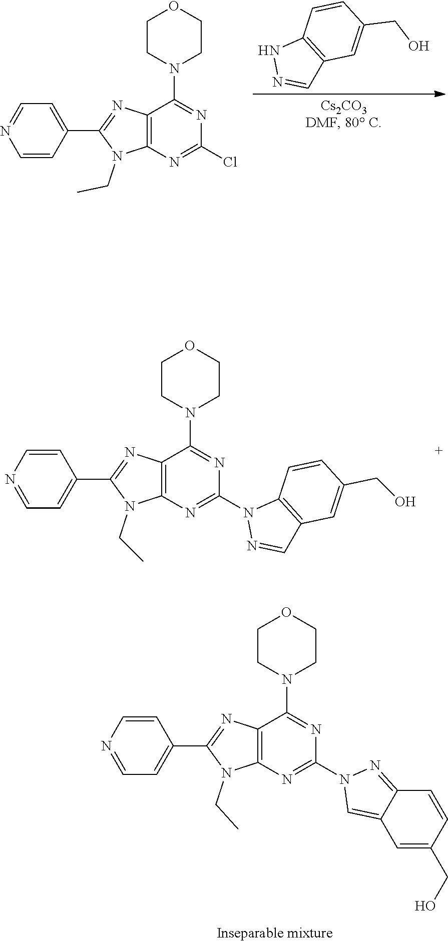 [SMILES: CCn1c(-c2ccncc2)nc2c(N3CCOCC3)nc(Cl)nc21 ; OCc1ccc2[nH]ncc2c1 ; CCn1c(-c2ccncc2)nc2c(N3CCOCC3)nc(-n3ncc4cc(CO)ccc43)nc21 ; CCn1c(-c2ccncc2)nc2c(N3CCOCC3)nc(-n3cc4cc(CO)ccc4n3)nc21]

To a solution of 4-[2-chloro-9-ethyl-8-(4-pyridyl)purin-6-yl]morpholine (100 mg, 0.26 mmol) in DMF (3 mL) were added 1H-indazol-5-ylmethanol (39 mg, 0.26 mmol) and Cs₂CO₃ (255 mg, 0.78 mmol). The resultant mixture was stirred at 80° C. for 12 h. It was concentrated under reduced pressure and the crude product was purified by prep-HPLC (Waters Xbridge BEH C18 100*30 mm*10 um column; 35-65% acetonitrile in an a 0.04% ammonia solution and an a 10 mM ammonium bicarbonate solution in water, 8 min gradient) to afford a mixture of [1-[9-ethyl-6-morpholino-8-(4-pyridyl)purin-2-yl]indazol-5-yl]methanol and (2-(9-ethyl-6-morpholino-8-(pyridin-4-yl)-9H-purin-2-yl)-2H-indazol-5-yl)methanol (19 mg, 19%) was obtained as a light yellow solid.

Note: The two isomers were inseparable under various conditions that were attempted.

¹H NMR (400 MHz, CHLOROFORM-d) δ 9.15-9.04 (m, 0.5H), 8.81 (bs, 2H), 8.70 (br d, J=8.8 Hz, 0.5H), 8.34-8.20 (m, 0.5H), 7.89-7.69 (m, 3H), 7.69-7.52 (m, 1H), 7.34 (br d, J=9.2 Hz, 0.5H), 4.85 (br d, J=12.0 Hz, 1H), 4.76 (br d, J=11.1 Hz, 1H), 4.65-4.27 (m, 6H), 3.92 (br d, J=3.8 Hz, 4H), 1.58-1.38 (m, 3H). LCMS (ESI for C24H24N8O2 [M+H]⁺: 457.2.

The following compounds were synthesized according to the protocol described for the Compound 200. The regioisomers were inseparable under various chromatographic conditions attempted.

| Name | Structure | NMR, MS | # |
|---|---|---|---|
| [1-[9-ethyl-6- morpholino-8- (4-pyridyl)purin-2- yl]indazol-6-yl] methanol and (2-(9-ethyl-6- morpholino-8- (pyridin-4-yl)-9H- purin-2-yl)-2H- indazol-6-yl) methanol | 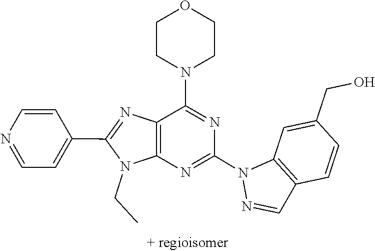 [SMILES: CCn1c(-c2ccncc2)nc2c(N3CCOCC3)nc(-n3ncc4ccc(CO)cc43)nc21] | ¹H NMR (400 MHz, CHLOROFORM-d) δ 9.11 (s, 0.5H), 8.80 (br d, J = 16.6 Hz, 2.5H), 8.26 (s, 0.5H), 7.84-7.60 (m, 3H), 7.13 (d, J = 8.6 Hz, 1.5H), 4.92 (s, 1H), 4.79 (s, 1H), 4.68-4.21 (m, 6H), 3.92 (br t, J = 4.6 Hz, 4H), 1.65-1.36 (m, 3H). LCMS (ESI for C24H24N802 [M + H]+: 457.2. | 201 |
| 4-[9-ethyl-2-(5- methoxyindazol- 1-yl)-8-(4- pyridyl)purin-6- yl]morpholine and 4-(9-ethyl- 2-(5-methoxy-2H- indazol-2-yl)-8- (pyridin-4- yl)-9H-purin-6- yl)morpholine | 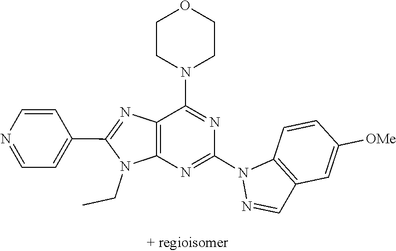 [SMILES: CCn1c(-c2ccncc2)nc2c(N3CCOCC3)nc(-n3ncc4cc(OC)ccc43)nc21] | ¹H NMR (400 MHz, CHLOROFORM-d) δ 8.97 (s, 0.5H), 8.90-8.79 (m, 2H), 8.62 (d, J = 9.1 Hz, 0.5H), 8.24 (s, 0.5H), 7.91-7.71 (m, 2.5H), 7.25- 7.17 (m, 1H), 7.09-6.99 (m, 0.5H), 6.89 (d, J = 2.1 Hz, 0.5H), 4.60-4.48 (m, 6), 3.99-3.85 (m, 7), 1.55-1.46 (m, 3H). LCMS (ESI for C24H24N802 [M + H]⁺: 457.1. | 202 |

The following compounds were synthesized according to the protocol described for the Compound 200. However, the regioisomers were separated successfully.

| Name | Structure | NMR, MS | # |
|---|---|---|---|
| 4-[9-ethyl-2-(6- methoxyindazol-1-yl)- 8-(4-pyridyl)purin-6- yl]morpholine | 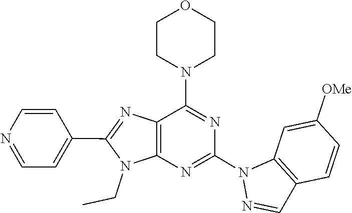 [SMILES: CCn1c(-c2ccncc2)nc2c(N3CCOCC3)nc(-n3ncc4ccc(OC)cc43)nc21] | ¹H NMR (400 MHz, CHLOROFORM-d) δ 8.82 (br s, 1H), 8.27 (d, J = 1.9 Hz, 1H), 8.20 (s, 1H), 7.72 (br s, 2H), 7.65 (d, J = 8.8 Hz, 1H), 6.95 (dd, J = 8.8, 2.3Hz, 1H), 4.54-4.50 (m, 6H), 3.95-3.91 (m, 7H), 1.69-1.34 (m, 3H). LCMS (ESI for C24H24N802 [M + H]⁺: 457.2. | 203 |
| 4-[9-ethyl-2-(5- methoxyindazol-2-yl)- 8-(4-pyridyl)purin-6- yl]morpholine | 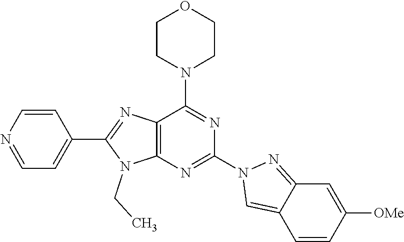 | ¹H NMR (400 MHz, CHLOROFORM-d) δ 9.03 (s, 1H), 8.83 (bs, 2H), 7.71 (d, J = 4.4 Hz, 2H), 7.56 (d, J = 9.2 Hz, 1H), 7.06 (s, 1H), 6.80 (dd, J = 9.1, 1.9Hz, 1H), 4.60- 4.25 (m, 6H), 3.92-3.74 (m, 7H), 1.48- 1.32 (m, 3H). LCMS (ESI for C24H24N802 [M + H]⁺: 457.2. | 204 |

### Synthesis of 1-(9-ethyl-2-(5-methyl-1H-indazol-1-yl)-6-morpholino-9H-purin-8-yl)ethan-1-ol (Compound 205) and 1-(9-ethyl-2-(5-methyl-2H-indazol-2-yl)-6-morpholino-9H-purin-8-yl)ethan-1-ol (Compound 206)

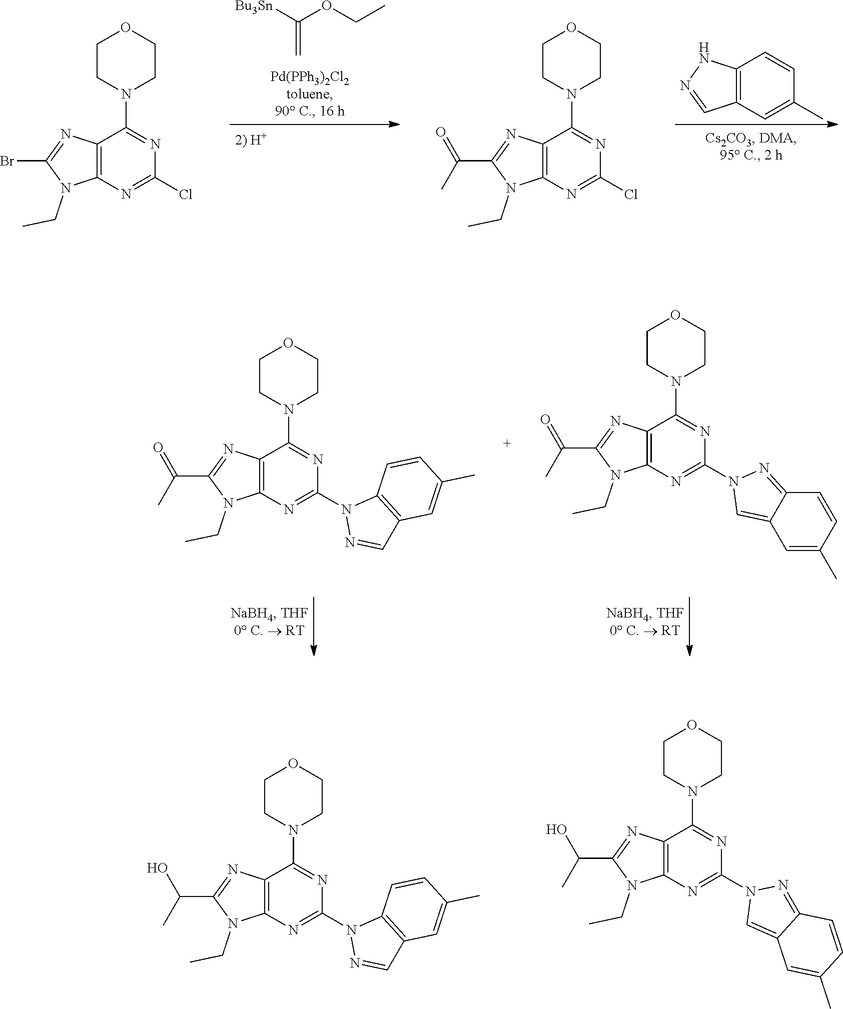

### Step 1: Synthesis of 1-(2-chloro-9-ethyl-6-morpholino-9H-purin-8-yl)ethan-1-one

A mixture of 4-(8-bromo-2-chloro-9-ethyl-9H-purin-6-yl)morpholine (2 g, 6 mmol), tributyl(1-ethoxyvinyl)stannane (2.38 g, 6.6 mmol) and 1,1′-bis(diphenylphosphino) ferrocene-palladium(II) dichloride dichloromethane complex (490 mg, 0.6 mmol) in toluene (15 mL) was stirred at 90° C. under nitrogen atmosphere for 16 h. Then HCl (6 mL, 3N) was added to the mixture and stirred for 30 min. The resultant mixture was concentrated and the crude product was purified by silica gel column (petroleum ether:acetic ester=92:8) to afford 1-(2-chloro-9-ethyl-6-morpholino-9H-purin-8-yl)ethan-1-one (800 mg, 43.3%) as white solid. LCMS (ESI) m/z: 310.2 [M+H]+.

### Step 2: Synthesis of 1-(9-ethyl-2-(5-methyl-1H-indazol-1-yl)-6-morpholino-9H-purin-8-yl)ethan-1-one and 1-(9-ethyl-2-(5-methyl-2H-indazol-2-yl)-6-morpholino-9H-purin-8-yl)ethan-1-one

A mixture of 1-(2-chloro-9-ethyl-6-morpholino-9H-purin-8-yl)ethan-1-one (400 mg, 1.3 mmol), 5-methyl-1H-indazole (205 mg, 1.55 mmol) and cesium carbonate (1271 mg, 3.9 mmol) in N,N-dimethylacetamide (2 mL) was stirred at 95° C. under argon atmosphere for 16 h. The reaction mixture was filtered, the filtrate was purified by prep-HPLC (Column Xbridge 21.2*250 mm C18, 10 um, Mobile Phase A: water (10 mmol/L ammonium bicarbonate) B: acetonitrile) to afford 1-(9-ethyl-2-(1H-indazol-1-yl)-6 morpholino-9H-purin-8-yl)ethan-1-one (40 mg, 8%) and 1-(9-ethyl-2-(2H-indazol-2-yl)-6 morpholino-9H-purin-8-yl)ethan-1-one (by-product, 30 mg, 6%). LCMS (ESI) m/z: 405.8 [M+H]+.

### Step 3: Synthesis of 1-(9-ethyl-2-(5-methyl-1H-indazol-1-yl)-6-morpholino-9H-purin-8-yl)ethan-1-ol

Sodium borohydride (6.4 mg, 0.17 mmol) was added to a solution of 1-(9-ethyl-2-(1H-indazol-1-yl)-6 morpholino-9H-purin-8-yl)ethan-1-one (40 mg, 0.1 mmol) in tetrahydrofuran (5 mL) at 0° C., and the mixture was stirred at room temperature for 1 h. It was filtered and the filtrate was purified by prep-HPLC (Column Xbridge 21.2*250 mm C18, 10 um, Mobile Phase A: water (10 mmol/L ammonium bicarbonate) B: acetonitrile) to afford 1-(9-ethyl-2-(5-methyl-1H-indazol-1-yl)-6-morpholino-9H-purin-8-yl)ethan-1-ol (5.8 mg, 8%) as white solid.

¹H NMR (400 MHz, DMSO) δ 8.52 (d, J=8.6 Hz, 1H), 8.28 (s, 1H), 7.64 (s, 1H), 7.40 (d, J=8.6 Hz, 1H), 5.72 (d, J=5.9 Hz, 1H), 5.03 (s, 1H), 4.40-4.20 (m, 6H), 3.82-3.74 (m, 4H), 2.45 (s, 3H), 1.58 (d, J=6.5 Hz, 3H), 1.44 (t, J=7.1 Hz, 3H); LCMS (ESI) m/z: 408.3 [M+H]+.

### Step 4: Synthesis of 1-(9-ethyl-2-(5-methyl-2H-indazol-2-yl)-6-morpholino-9H-purin-8-yl)ethan-1-ol

Compound 206 was synthesized similar to Compound 205.

¹H NMR (400 MHz, DMSO) δ 9.14 (s, 1H), 7.66 (d, J=8.9 Hz, 1H), 7.50 (s, 1H), 7.16 (dd, J=9.0, 1.3 Hz, 1H), 5.74 (d, J=6.2 Hz, 1H), 5.04 (pent, J=6.4 Hz, 1H), 4.45-4.25 (m, 6H), 3.89-3.67 (m, 4H), 2.37 (s, 3H), 1.58 (d, J=6.5 Hz, 3H), 1.42 (t, J=7.1 Hz, 3H); LCMS (ESI) m/z: 408.3 [M+H]+.

### Synthesis of 1-(9-methyl-6-morpholino-8-(pyridin-4-yl)-9H-purin-2-yl)-4-phenyl-1H-pyrazol-5-ol (Compound 207)

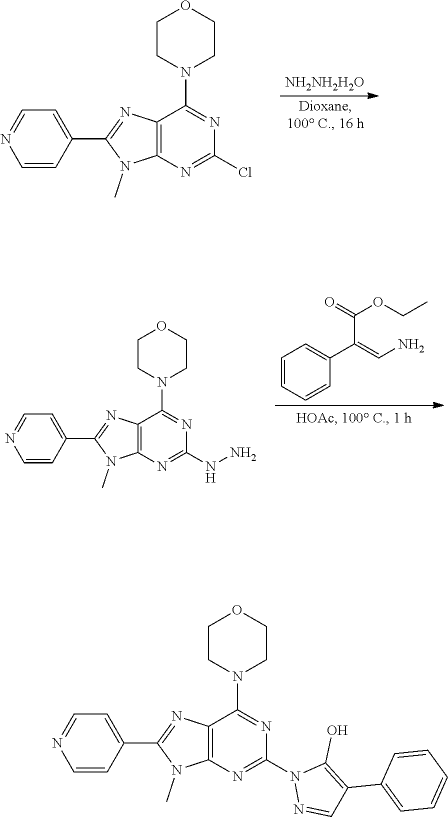 [SMILES: Cn1c(-c2ccncc2)nc2c(N3CCOCC3)nc(Cl)nc21 ; "NH2NH2H2O" ; "Dioxane,100° C., 16 h" ; Cn1c(-c2ccncc2)nc2c(N3CCOCC3)nc(NN)nc21 ; CCOC(=O)/C(=C\N)c1ccccc1 ; Cn1c(-c2ccncc2)nc2c(N3CCOCC3)nc(-n3ncc(-c4ccccc4)c3O)nc21]

### Step 1: Preparation of 4-(2-hydrazineyl-9-methyl-8-(pyridin-4-yl)-9H-purin-6-yl)morpholine

A mixture of 4-(2-chloro-9-methyl-8-(pyridin-4-yl)-9H-purin-6-yl)morpholine (0.2 g, 0.6 mmol) and hydrazine hydrate (1 mL) in dioxane (12 mL) was stirred at 100° C. for 16 h. The reaction mixture was concentrated to give product (0.197 g, 100%) as a yellow solid. LCMS (ESI) m/z: 327.0 [M+H]⁺.

### Step 2: Preparation of 1-(9-methyl-6-morpholino-8-(pyridin-4-yl)-9H-purin-2-yl)-4-phenyl-1H-pyrazol-5-ol

A mixture of 4-(2-hydrazineyl-9-methyl-8-(pyridin-4-yl)-9H-purin-6-yl)morpholine (0.1 g, 0.3 mmol) and ethyl (Z)-3-amino-2-phenylacrylate (0.12 g, 0.6 mmol) in AcOH (3 mL) was stirred at 100° C. under N2 for 1 h. The reaction mixture was concentrated and purified by prep-HPLC (SunFire C18, 4.6*50 mm, 3.5 um column Xbridge C18 3.5 μm 4.6×50 mm column. The mobile phase was acetonitrile/10 mM formic acid aqueous solution) to give the desired product (0.0594 g, 0.13 mmol, 43%) as a yellow solid. LCMS (ESI) m/z: 455.2 [M+H]⁺. ¹H NMR (400 MHz, DMSO-d₆) δ 13.21 (s, 1H), 8.79 (d, J=5.9 Hz, 2H), 8.10 (s, 1H), 7.91 (d, J=6.0 Hz, 2H), 7.77 (s, 2H), 7.38 (t, J=7.6 Hz, 2H), 7.18 (t, J=7.2 Hz, 1H), 4.22 (s, 4H), 3.94 (s, 3H), 3.82 (s, 4H).

The following compounds were synthesized according to the protocol described above:

| Name | Structure | NMR, MS | # |
|---|---|---|---|
| 1-(9-ethyl-6- morpholino-8- (pyridin-4-yl)-9H- purin-2-yl)-4- phenyl-1H-pyrazol- 5-ol | 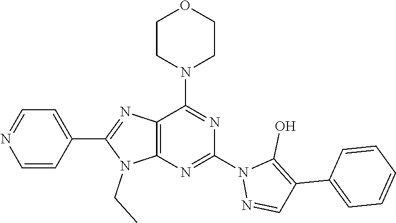 [SMILES: CCn1c(-c2ccncc2)nc2c(N3CCOCC3)nc(-n3ncc(-c4ccccc4)c3O)nc21] | ¹H NMR (400 MHz, DMSO-d₆) δ 8.80 (d, J = 6.0 Hz, 2H), 8.13 (s, 1H), 7.84 (d, J = 6.0 Hz, 2H), 7.80 (d, J = 7.5 Hz, 2H), 7.37 (t, J = 7.7 Hz, 2H), 7.16 (t, J = 7.4 Hz, 1H), 4.41 (dd, J = 14.2, 7.1 Hz, 6H), 3.82 (d, J = 4.3 Hz, 4H), 1.36 (t, J = 7.2 Hz, 3H); LCMS (ESI) m/z: 469.2 [M + H]⁺. | 208 |
| 1-(9-methyl-6- morpholino-8- (pyridin-4-yl)-9H- purin-2-yl)-4- phenyl-1H-pyrazol- 3-ol | 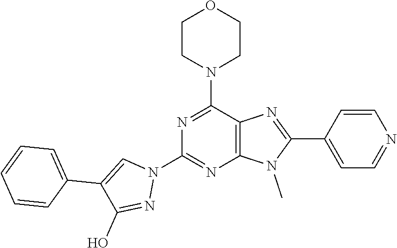 | 1H NMR (400 MHz, DMSO-d6) δ 8.91 (s, 1H), 8.78 (d, J = 6.0 Hz, 2H), 7.96- 7.86 (m, 5H), 7.39 (t, J = 7.8 Hz, 2H), 7.23 (s, 1H), 4.52-4.21 (m, 4H), 3.92 (s, 3H), 3.80 (s, 4H); LCMS (ESI) m/z: 455.2 [M + H]⁺ | 209 |
| (R)-1-(9-ethyl-6-(3- methylmorpholino)- 8-(pyridin-4-yl)-9H- purin-2-yl)-4- phenyl-1H-pyrazol- 3-ol | 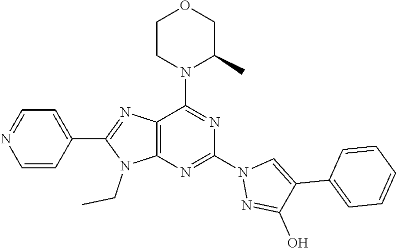 [SMILES: CCn1c(-c2ccncc2)nc2c(N3CCOC[C@H]3C)nc(-n3cc(-c4ccccc4)c(O)n3)nc21] | ¹H NMR (400 MHz, DMSO-d₆) δ 11.46 (s, 1H), 8.87 (s, 1H), 8.79 (d, J = 6.0 Hz, 2H), 7.88-7.83 (m, 4H), 7.39 (t, J = 7.7 Hz, 2H), 7.23 (t, J = 7.2 Hz, 1H), 4.38 (s, 2H), 4.38 (d, J = 7.0 Hz, 2H), 4.03 (d, J = 7.9 Hz, 1H), 3.83 (d, J = 8Hz, 2H), 3.74 (d, J = 8 Hz, 2H), 3.57 (q, J = 8 Hz, 2H), 1.40-1.31 (m, 6H); LCMS (ESI) m/z: 483.2 [M + H]⁺ | 210 |
| (S)-1-(9-ethyl-6-(3- methylmorpholino)- 8-(pyridin-4-yl)-9H- purin-2-yl)-4- phenyl-1H-pyrazol- 3-ol | 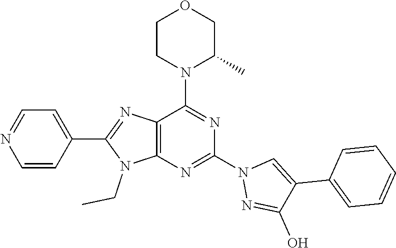 [SMILES: CCn1c(-c2ccncc2)nc2c(N3CCOC[C@@H]3C)nc(-n3cc(-c4ccccc4)c(O)n3)nc21] | ¹H NMR (400 MHz, DMSO-d₆) δ 11.47 (s, 1H), 8.87 (s, 1H), 8.82-8.77 (m, 2H), 7.89-7.80 (m, 4H), 7.39 (t, J = 7.8 Hz, 2H), 7.23 (t, J = 7.4 Hz, 1H), 4.38 (d, J = 7.4 Hz, 2H), 4.38 (d, J = 7.4 Hz, 2H), 4.03 (d, J = 8.3 Hz, 1H), 3.83 (d, J = 12 Hz, 2H), 3.75 (d, J = 12 Hz, 2H), 1.39-1.32 (m, 6H); LCMS (ESI) m/z: 482.9 [M+]⁺ | 211 |

### Preparation of 1-(9-ethyl-6-morpholino-8-(pyridin-4-yl)-9H-purin-2-yl)-3-phenyl-1H-pyrazol-5-ol (Compound 212)

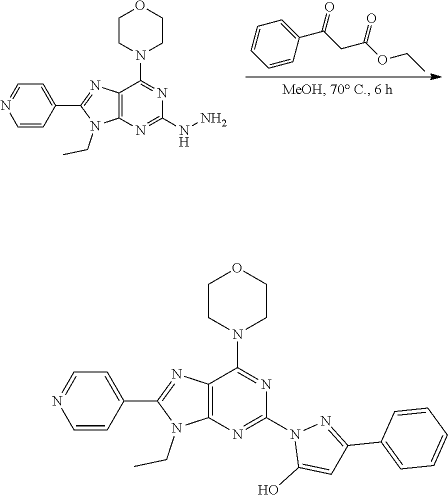 [SMILES: CCn1c(-c2ccncc2)nc2c(N3CCOCC3)nc(NN)nc21 ; CCOC(=O)CC(=O)c1ccccc1 ; CCn1c(-c2ccncc2)nc2c(N3CCOCC3)nc(-n3nc(-c4ccccc4)cc3O)nc21]

A mixture of 4-(9-ethyl-2-hydrazineyl-8-(pyridin-4-yl)-9H-purin-6-yl)morpholine (100 mg, 0.3 mmol) and ethyl 3-oxo-3-phenylpropanoate (56 mg, 0.3 mmol) in methanol (2 mL) was stirred at reflux for 16 h. The reaction mixture concentrated and the crude product was purified by column chromatography on silica gel (dichloromethane/methanol 20:1-*10:1-*5:1) to give 1-(9-ethyl-6-morpholino-8-(pyridin-4-yl)-9H-purin-2-yl)-3-phenyl-1H-pyrazol-5-ol (80 mg, 58%) as a white solid.

¹H NMR (400 MHz, DMSO-d₆) δ 12.28 (s, 1H), 8.81 (d, J=5.7 Hz, 2H), 7.86 (dd, J=14.2, 6.6 Hz, 4H), 7.45 (t, J=7.4 Hz, 2H), 7.38 (d, J=7.3 Hz, 1H), 6.17 (s, 1H), 4.41 (dd, J=14.3, 7.1 Hz, 6H), 3.82 (d, J=4.5 Hz, 4H), 1.36 (t, J=7.2 Hz, 3H); LCMS (ESI) m/z: 468.9 [M+H]+.

### Synthesis of 1-(9-ethyl-6-morpholino-8-(pyridin-4-yl)-9H-purin-2-yl)-4-phenyl-1H-pyrazol-3-ol (Compound 213)

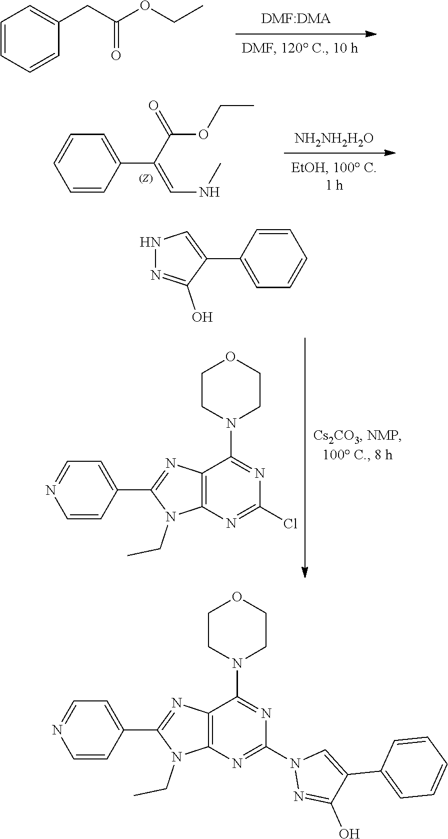

### Step 1: Preparation of ethyl (E)-3-(dimethylamino)-2-phenylacrylate

A mixture of ethyl 2-phenylacetate (3.0 g, 18.3 mmol) and 1,1-dimethoxy-N,N-dimethylmethanamine (0.94 g, 91.4 mmol) in N,N-dimethylformamide (30 mL) was stirred at 120° C. for 10 h. The reaction mixture was diluted with water (25 mL) and extracted with ethyl acetate (25 mL*3), the organic layer was concentrated to give the crude product (2 g, 50%) as an yellow oil. LCMS (ESI) m/z: 220.0 [M+H]⁺.

### Step 2: Preparation of 4-phenyl-1H-pyrazol-3-ol

A mixture of ethyl (Z)-3-(dimethylamino)-2-phenylacrylate (2 g, 9.12 mmol) and hydrazine hydrate (2.3 g, 45.6 mmol) in ethanol (20 mL) was stirred at 100° C. for 1 h. The reaction mixture was concentrated and purified by silica gel column (petroleum ether:acetic ester=2:1) to give the desired product (1 g, 68%) as off-white solid. LCMS (ESI) m/z: 161.0 [M+H]⁺.

### Step 3: Synthesis of 1-(9-ethyl-6-morpholino-8-(pyridin-4-yl)-9H-purin-2-yl)-4-phenyl-1H-pyrazol-3-ol

A mixture of 4-(2-chloro-9-ethyl-8-(pyridin-4-yl)-9H-purin-6-yl)morpholine (100 mg, 0.29 mmol), cesium carbonate (190 mg, 0.58 mmol) and 4-phenyl-1H-pyrazol-3-ol (56 mg, 0.35 mmol) in 1-methyl-2-pyrrolidinone (4 mL) was stirred at 100° C. for 8 h. The resultant reaction mixture was filtered and purified by prep-HPLC (SunFire C18, 4.6*50 mm, 3.5 um column Xbridge C18 3.5 μm 4.6×50 mm column. The mobile phase was acetonitrile/10 mM trifluoroacetic acid aqueous solution.) to give the desired product (17.6 mg, 13%) as off-white solid. ¹H NMR (400 MHz, DMSO) δ 8.90 (s, 1H), 8.82 (d, J=6.0 Hz, 2H), 7.90 (d, J=6.3 Hz, 3H), 7.86 (s, 1H), 7.39 (t, J=7.7 Hz, 2H), 7.24 (d, J=7.3 Hz, 1H), 4.56-4.23 (m, 6H), 3.80 (d, J=4.5 Hz, 4H), 1.34 (t, J=7.2 Hz, 3H); LCMS (ESI) m/z: 468.9 [M+H]⁺.

The following compound was synthesized according to the protocol described above:

| Name | Structure | NMR, MS | # |
|---|---|---|---|
| 1-(9-ethyl-6-(3- methylmorpholino)- 8-(pyridin-4-yl)-9H- purin-2-yl)-4- phenyl-1H-pyrazol- 3-ol | 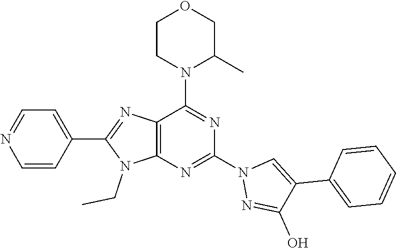 [SMILES: CCn1c(-c2ccncc2)nc2c(N3CCOCC3C)nc(-n3cc(-c4ccccc4)c(O)n3)nc21] | 1H NMR (400 MHz, DMSO-d6) δ 11.50 (s, 1H), 8.89 (s, 1H), 8.79 (d, J = 5.2 Hz, 2H), 7.86 (dd, J = 13.9, 6.3 Hz, 4H), 7.39 (t, J = 7.5 Hz, 2H), 7.23 (t, J = 7.2 Hz, 1H), 6.11-5.20 (m, 1H), 4.39 (s, 1H), 4.37 (q, J = 6.8 Hz, 2H), 4.03 (d, J = 8.6 Hz, 1H), 3.78 (dd, J = 36.5, 11.3 Hz, 2H), 3.57 (d, J = 11.0 Hz, 1H), 1.39-1.31 (m, 6H); LCMS (ESI) m/z: 483.2 [M + H]⁺ | 214 |

### Synthesis of 4-(9-ethyl-2-(3-methoxy-4-phenyl-1H-pyrazol-1-yl)-8-(pyridin-4-yl)-9H-purin-6-yl)morpholine (Compound 215)

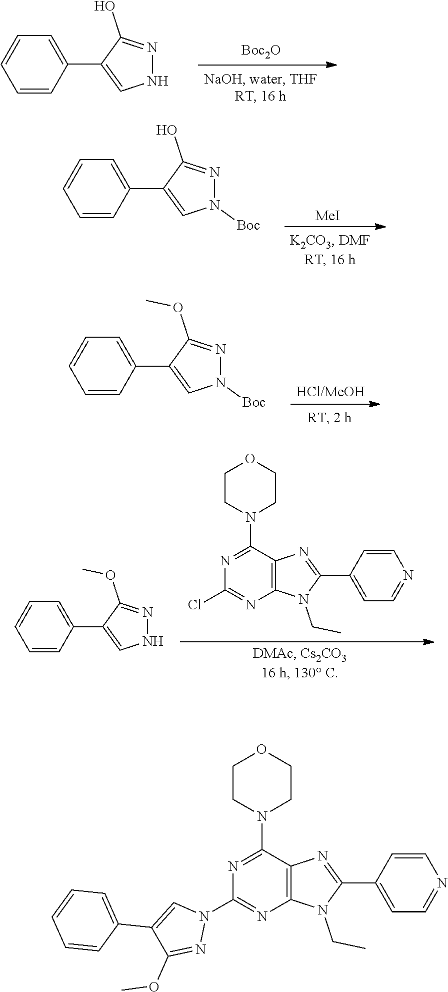

### Step 1: tert-butyl 3-hydroxy-4-phenyl-1H-pyrazole-1-carboxylate

A mixture of 4-phenyl-1H-pyrazol-3-ol (200 mg, 1.25 mmol), di-tert-butyl dicarbonate (545 mg, 2.5 mmol) and sodium hydroxide (2.5 mol/L in water, 2 mL) in tetrahydrofuran (2 mL) was stirred at room temperature for 16 h. The residue was partitioned between ethyl acetate (50 mL) and water (50 mL). The organic layer was washed with brine, dried and evaporated to dryness. The crude product was purified by flash chromatography on silica gel (Petroleum ether/Ethyl acetate 20:1->10:1->5:1) to give tert-butyl 3-hydroxy-4-phenyl-1H-pyrazole-1-carboxylate (220 mg, 67%) as a white solid.. LCMS (ESI) m/z: 205.1 [M+−55]⁺.

### Step 2: tert-butyl 3-methoxy-4-phenyl-1H-pyrazole-1-carboxylate

A mixture of tert-butyl 3-hydroxy-4-phenyl-1H-pyrazole-1-carboxylate (200 mg, 0.76 mmol), iodomethane (214 mg, 1.52 mmol) and potassium carbonate (209 mg, 1.52 mmol) in N,N-dimethylformamide (2 mL) was stirred at room temperature for 16 h. The residue was partitioned between ethyl acetate (50 mL) and water (50 mL). The organic layer was washed with brine, dried and evaporated to dryness. The crude product was purified by flash chromatography on silica gel (Petroleum ether/Ethyl acetate 20:1->10:1) to give the tert-butyl 3-methoxy-4-phenyl-1H-pyrazole-1-carboxylate (190 mg, 90%) as a white solid. LCMS (ESI) m/z: 219.1 [M+−55]⁺.

### Step 3: 3-methoxy-4-phenyl-1H-pyrazole

A mixture of tert-butyl 3-methoxy-4-phenyl-1H-pyrazole-1-carboxylate (100 mg, 0.3 mmol) in hydrochloric acid (3 mol/L in methanol, 5 mL) was stirred at room temperature for 2 h. The mixture was evaporated to afford 3-methoxy-4-phenyl-1H-pyrazole (70 mg, 75%) as a colorless oil. LCMS (ESI) m/z: 175.1 [M+H]⁺.

### Step 4: 4-(9-ethyl-2-(3-methoxy-4-phenyl-1H-pyrazol-1-yl)-8-(pyridin-4-yl)-9H-purin-6-yl)morpholine

A mixture of 4-(2-chloro-9-ethyl-8-(pyridin-4-yl)-9H-purin-6-yl)morpholine (60 mg, 0.17 mmol), 3-methoxy-4-phenyl-1H-pyrazole (40 mg, 0.22 mmol) and cesium carbonate (165 mg, 0.51 mmol) in N,N-dimethylacetamide (2 mL) was stirred at 130° C. for 16 h. The residue was partitioned between ethyl acetate (50 mL) and water (50 mL). The organic layer was dried and concentrated. The residue was purified by prep-HPLC to afford 4-(9-ethyl-2-(3-methoxy-4-phenyl-1H-pyrazol-1-yl)-8-(pyridin-4-yl)-9H-purin-6-yl)morpholine (7.6 mg, 9.8%) as a white solid. 1H NMR (400 MHz, DMSO-d₆) δ 8.98 (s, 1H), 8.79 (d, J=6.0 Hz, 2H), 7.86-7.82 (m, 4H), 7.41 (t, J=7.7 Hz, 2H), 7.26 (t, J=7.4 Hz, 1H), 4.65-4.25 (m, 6H), 4.09 (s, 3H), 3.80 (s, 4H), 1.33 (t, J=7.2 Hz, 3H); LCMS (ESI) m/z: 483.1 [M+H]⁺.

### Synthesis of 4-(9-ethyl-2-(3-methoxy-4-phenyl-1H-pyrazol-1-yl)-9H-purin-6-yl)morpholine (Compound 216)

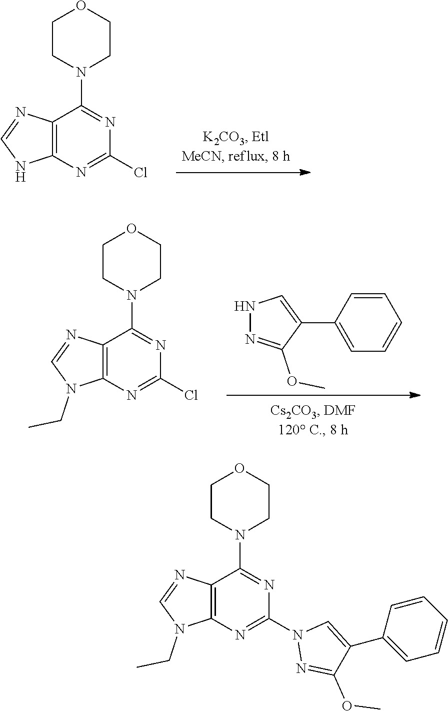 [SMILES: Clc1nc(N2CCOCC2)c2nc[nH]c2n1 ; CCn1cnc2c(N3CCOCC3)nc(Cl)nc21 ; COc1n[nH]cc1-c1ccccc1 ; CCn1cnc2c(N3CCOCC3)nc(-n3cc(-c4ccccc4)c(OC)n3)nc21]

### Step 1: Synthesis of 4-(2-chloro-9-ethyl-9H-purin-6-yl)morpholine

To a solution of 4-(2-chloro-9H-purin-6-yl)morpholine (7.3 g, 0.0309 mol) in acetonitrile (100 mL) were added iodoethane (7.1 g, 45.8 mmol) and potassium carbonate (8.6 g, 61.8 mmol). The resultant reaction mixture was stirred at 90° C. for 8 h and then quenched with water (15 mL) and extracted with ethyl acetate (20*3 mL). The organic layer was combined, washed with brine (30 mL), dried over sodium sulfate, filtered and concentrated. The crude product thus obtained was purified by flash chromatography on silica gel (petroleum ether:ethyl acetate=75:25) to obtain the target compound as yellow solid. (5.4 g, 40.5% yield).

### Step 2: Synthesis of 4-(9-ethyl-2-(3-methoxy-4-phenyl-1H-pyrazol-1-yl)-9H-purin-6-yl)morpholine

To a solution of 4-(2-chloro-9-ethyl-9H-purin-6-yl)morpholine (133 mg, 0.50 mmol) in N,N-dimethylformamide (5 mL) were added 3-methoxy-4-phenyl-1H-pyrazole (170 mg, 0.50 mmol) and cesium carbonate (32 mg, 1.0 mmol). The resultant mixture was stirred at 120° C. for 8 h, then quenched with water (15 mL) and extracted with ethyl acetate (20*3 mL). The organic layers were combined, washed with brine (30 mL), dried over sodium sulfate, filtered and concentrated. The crude product was purified by prep-HPLC (0.05% NH4HCO3/H2O:CH3CN=5%-95%) to obtain 4-(9-ethyl-2-(3-methoxy-4-phenyl-1H-pyrazol-1-yl)-9H-purin-6-yl)morpholine (50 mg, 35.6%) as white solid.

¹H NMR (400 MHz, DMSO-d₆) δ 8.94 (s, 1H), 8.21 (s, 1H), 7.82 (d, J=7.3 Hz, 2H), 7.40 (t, J=7.7 Hz, 2H), 7.25 (t, J=7.4 Hz, 1H), 4.73-4.11 (m, 6H), 4.07 (s, 3H), 3.86-3.63 (m, 4H), 1.45 (t, J=7.3 Hz, 3H); LCMS (ESI) m/z: 405.8[M+]+.

### Synthesis of 1-(9-ethyl-2-(3-methoxy-4-phenyl-1H-pyrazol-1-yl)-6-morpholino-9H-purin-8-yl)cyclobutanol (Compound 217)

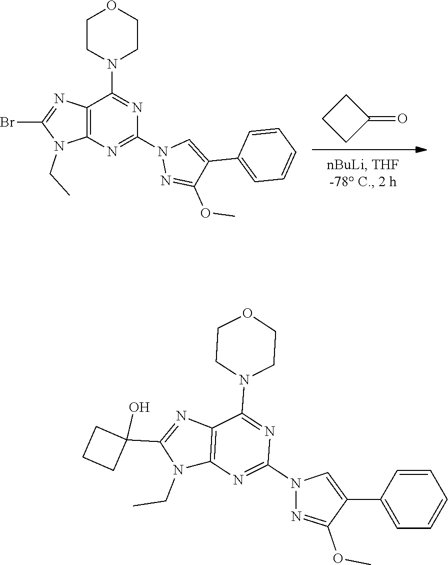

To a stirred solution of 4-(8-bromo-9-ethyl-2-(3-methoxy-4-phenyl-1H-pyrazol-1-yl)-9H-purin-6-yl)morpholine (73 mg, 0.15 mmol) in tetrahydrofuran (5 mL) at −78° C., was added 1.6 M n-butyllithium in hexane (0.19 mL, 0.3 mmol) and the reaction mixture was stirred for 10 minutes. Cyclobutanone (21 mg, 0.3 mmol) was added dropwise and the mixture was stirred at −78° C. for another 2 h. The reaction mixture was quenched with saturated aqueous ammonium chloride solution (10 mL), then diluted with water (20 mL) and extracted with dichloromethane (50 mL*3). The organic layer was dried over sodium sulfate and concentrated under reduced pressure to afford a residue, which was purified by pre-HPLC (BOSTON pHlex ODS 10 μm 21.2×250 mm 120 A. The mobile phase was acetonitrile/0.1% Ammonium bicarbonate) to obtain 1-(9-ethyl-2-(3-methoxy-4-phenyl-1H-pyrazol-1-yl)-6-morpholino-9H-purin-8-yl)cyclobutanol (17.2 mg, 18%) as white solid.

¹H NMR (500 MHz, DMSO-d₆) δ 8.91 (s, 1H), 7.81 (d, J=7.6 Hz, 2H), 7.40 (t, J=7.7 Hz, 2H), 7.25 (t, J=7.4 Hz, 1H), 6.19 (s, 1H), 4.45-4.20 (m, 6H), 4.07 (s, 3H), 3.82-3.74 (m, 4H), 2.90-2.70 (m, 2H), 2.33 (dd, J=16.4, 7.6 Hz, 2H), 1.92-1.83 (m, 1H), 1.63 (pent, 10.1 Hz, 1H), 1.41 (t, J=7.1 Hz, 3H); LCMS: (ESI) m/z: 475.8 [M+]⁺.

### Synthesis of 4-(8-cyclopropyl-9-ethyl-2-(3-methoxy-4-phenyl-1H-pyrazol-1-yl)-9H-purin-6-yl)morpholine (Compound 218)

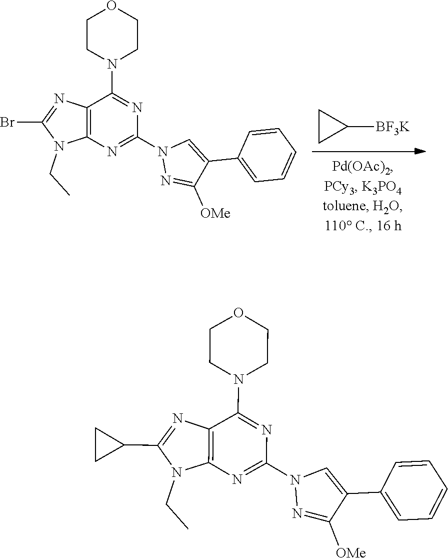

A mixture of 4-(8-bromo-9-ethyl-2-(3-methoxy-4-phenyl-1H-pyrazol-1-yl)-9H-purin-6-yl)morpholine (150 mg, 0.310 mmol), potassium cyclopropyltrifluoroborate (92 mg, 0.620 mmol), acetoxypalladium (30 mg, 0.05 mmol), 2-tricyclohexylphosphane (56 mg, 0.06 mmol) and potassium phosphate (131 mg, 0.620 mmol) in toluene (5 mL) and water (5 mL) was stirred at 110° C. for 16 h. The reaction mixture was cooled, then quenched with water (15 mL) and extracted with ethyl acetate (20*3 mL). The organic layer was combined, washed with brine (30 mL), dried over sodium sulfate, filtered and concentrated. The residue was purified by Prep-HPLC (0.05% NH4HCO3/H2O:CH3CN=5%-95%) to afford 4-(8-cyclopropyl-9-ethyl-2-(3-methoxy-4-phenyl-1H-pyrazol-1-yl)-9H-purin-6-yl)morpholine (16.1 mg, 40%) as white solid. ¹H NMR (400 MHz, DMSO-d₆) δ 8.92 (d, J=11.7 Hz, 1H), 7.81 (d, J=8 Hz, 2H), 7.39 (t, J=7.7 Hz, 2H), 7.24 (t, J=7.4 Hz, 1H), 4.35 (q, J=7.2 Hz, 2H), 4.24 (bs, 4H), 4.07 (s, 3H), 3.74 (t, J=4 Hz, 4H), 2.24 (pent, J=5.0 Hz, 1H), 1.39 (t, J=7.2 Hz, 3H), 1.17-0.86 (m, 4H); LCMS (ESI) m/z: 445.8[M+]+.

### Synthesis of 2-(9-ethyl-2-(3-methoxy-4-phenyl-1H-pyrazol-1-yl)-6-morpholino-9H-purin-8-yl)ethan-1-ol (Compound 219)

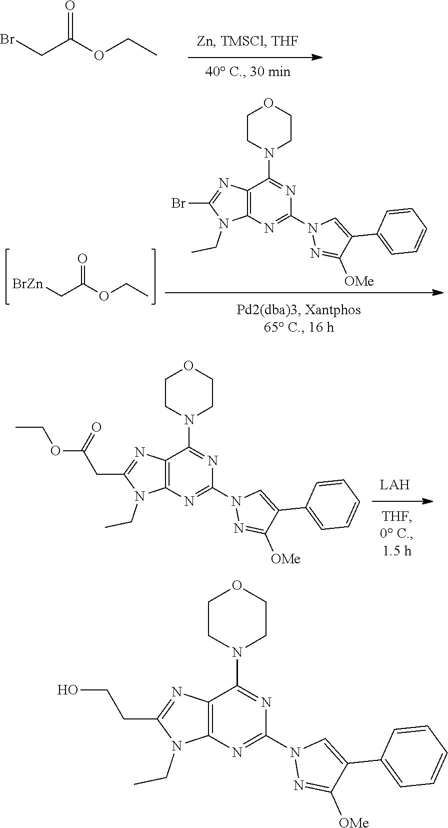

### Step 1: Synthesis of ethyl 2-(9-ethyl-2-(3-methoxy-4-phenyl-1H-pyrazol-1-yl)-6-morpholino-9H-purin-8-yl)acetate

Under nitrogen atmosphere, zinc powder (2.56 g, 39.15 mmol) was suspended in tetrahydrofuran (20 mL) and trimethylsilyl chloride (0.25 mL, 2.9 mmol) was added thereto at room temperature and the resultant mixture was stirred for 30 min. The reaction mixture was then heated to 40° C. and ethyl 2-bromoacetate (2.2 mL, 19.65 mmol) was added drop-wise to the mixture and stirred at 40° C. for further 30 min. After insoluble matter precipitated, the light-yellow supernatant solution was decanted and used for subsequent experiments.

To a solution of 4-(8-bromo-9-ethyl-2-(3-methoxy-4-phenyl-1H-pyrazol-1-yl)-9H-purin-6-yl)morpholine (0.4 g, 0.83 mmol), tris(dibenzylideneacetone)dipalladium (76 mg, 0.08 mmol) and 4,5-bis(diphenylphosphino)-9,9-dimethylxanthene (96 mg, 0.16 mmol) in tetrahydrofuran (20 mL) was added the above zinc bromide solution (2.5 mL, 2.5 mmol) drop-wise at room temperature under argon. After the addition, the reaction mixture was heated 65° C. and stirred for 16 h. The reaction mixture was cooled down, quenched by ammonium chloride aqueous solution and extracted with ethyl acetate (20 mL×2).

The combined organic phase was washed with brine (20 mL), dried over sodium sulfate, filter and concentrated. The residue was purified by flash chromatography (eluted with ethyl acetate in petro ether from 20% to 40%) to afford ethyl 2-(9-ethyl-2-(3-methoxy-4-phenyl-1H-pyrazol-1-yl)-6-morpholino-9H-purin-8-yl)acetate (0.35 g, 85.9%) as yellow solid. LCMS (ESI) m/z: 491.8 [M+]⁺.

### Step 2: Preparation of 2-(9-ethyl-2-(3-methoxy-4-phenyl-1H-pyrazol-1-yl)-6-morpholino-9H-purin-8-yl)ethan-1-ol

To a solution of ethyl 2-(9-ethyl-2-(3-methoxy-4-phenyl-1H-pyrazol-1-yl)-6-morpholino-9H-purin-8-yl)acetate (0.1 g, 0.2 mmol) in tetrahydrofuran (15 mL) which is cooled to 0° C., was added a 1M lithium aluminum hydride solution in tetrahydrofuran (0.6 mL, 0.6 mmol) drop-wise under nitrogen. After the addition, the reaction was stirred at 0° C. for 1.5 h. The reaction was quenched by sodium sulfate decahydrate and filtered. The filtrate was concentrated and the residue was purified by PREP-HPLC (base) to afford 2-(9-ethyl-2-(3-methoxy-4-phenyl-1H-pyrazol-1-yl)-6-morpholino-9H-purin-8-yl)ethan-1-ol (25 mg, 27.8%) as white solid.

¹H NMR (500 MHz, DMSO-d₆) δ 8.91 (s, 1H), 7.81 (d, J=7.9 Hz, 2H), 7.39 (t, J=7.7 Hz, 2H), 7.24 (t, J=7.3 Hz, 1H), 4.35-4.15 (m, 6H), 4.07 (s, 3H), 3.83 (t, J=6.6 Hz, 2H), 3.79-3.71 (m, 4H), 3.00 (t, J=6.6 Hz, 2H), 1.36 (t, J=7.2 Hz, 3H); LCMS (ESI) m/z: 449.8 [M+]⁺.

### Preparation of ethyl 3-cyclopropyl-1-(9-ethyl-6-morpholino-8-(pyridin-4-yl)-9H-purin-2-yl)-1H-pyrazole-5-carboxylate (Compound 220), 3-cyclopropyl-1-(9-ethyl-6-morpholino-8-(pyridin-4-yl)-9H-purin-2-yl)-1H-pyrazole-5-carboxylic acid (Compound 221) and (3-cyclopropyl-1-(9-ethyl-6-morpholino-8-(pyridin-4-yl)-9H-purin-2-yl)-1H-pyrazol-5-yl)methanol (Compound 222)

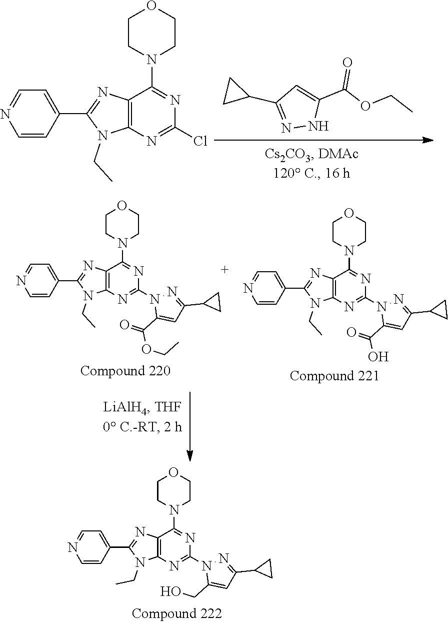

### Step 1: Synthesis of ethyl 3-cyclopropyl-1-(9-ethyl-6-morpholino-8-(pyridin-4-yl)-9H-purin-2-yl)-1H-pyrazole-5-carboxylate and 3-cyclopropyl-1-(9-ethyl-6-morpholino-8-(pyridin-4-yl)-9H-purin-2-yl)-1H-pyrazole-5-carboxylic acid

A mixture of 4-(2-chloro-9-ethyl-8-(pyridin-4-yl)-9H-purin-6-yl)morpholine (68 mg, 0.2 mmol), ethyl 3-cyclopropyl-1H-pyrazole-5-carboxylate (43 mg, 0.24 mmol) and cesium carbonate (196 mg, 0.6 mmol) in dry N,N-dimethylacetamide (4 mL) was stirred at 120° C. for 16 h. The crude products formed were purified by pre-HPLC (BOSTON pHlex ODS 10 μm 21.2×250 mm 120 A. The mobile phase was acetonitrile/0.1% Ammonium bicarbonate) to get ethyl 3-cyclopropyl-1-(9-ethyl-6-morpholino-8-(pyridin-4-yl)-9H-purin-2-yl)-1H-pyrazole-5-carboxylate (Compound 220) (71.4 mg, 0.146 mmol, 49%) and 3-cyclopropyl-1-(9-ethyl-6-morpholino-8-(pyridin-4-yl)-9H-purin-2-yl)-1H-pyrazole-5-carboxylic acid (Compound 221) (14.2 mg, 0.031 mmol, 10%) as white solids.

Compound 220: 1H NMR (400 MHz, DMSO-d₆) δ 8.81 (d, J=5.9 Hz, 2H), 7.85 (d, J=6.0 Hz, 2H), 6.58 (s, 1H), 4.87-3.91 (m, 8H), 3.77 (s, 4H), 2.46-2.39 (m, 1H), 1.40-1.26 (m, 6H), 1.01-0.90 (m, 2H), 0.83-0.72 (m, 2H); LCMS, [M+H]+=488.8.

Compound 221: 1 H NMR (400 MHz, DMSO d₆) δ 8.81 (d, J=6.0 Hz, 2H), 7.85 (d, J=6.0 Hz, 2H), 6.42 (s, 1H), 4.39 (dd, J=14.2, 7.0 Hz, 6H), 3.77 (s, 4H), 2.44 (dd, J=8.3, 5.2 Hz, 1H), 1.34 (t, J=7.2 Hz, 3H), 0.99-0.89 (m, 2H), 0.78-0.69 (m, 2H); LCMSA011, [M+H]+=460.8.

### Step 2: Procedure for compound (3-cyclopropyl-1-(9-ethyl-6-morpholino-8-(pyridin-4-yl)-9H-purin-2-yl)-1H-pyrazol-5-yl)methanol (Compound 222)

Ethyl 3-cyclopropyl-1-(9-ethyl-6-morpholino-8-(pyridin-4-yl)-9H-purin-2-yl)-1H-pyrazole-5-carboxylate (50 mg, 0.1 mmol) in anhydrous tetrahydrofuran (5 mL) was added to a solution of lithium aluminum hydride (0.5 mL, 0.5 mmol, 1 M in THF) at 0° C. and stirred under nitrogen for 2 h. The reaction was quenched by careful addition of sodium sulfate decahydrate with ice-bath cooling. Tetrahydrofuran (50 mL) was added to the reaction mixture, the mixture was stirred for 15 min. The solid was filtered and washed with tetrahydrofuran (50 mL). The combined filtrates were evaporated in vacuo. The residue was purified by pre-HPLC (BOSTON pHlex ODS 10 μm 21.2×250 mm 120 A. The mobile phase was acetonitrile/0.1% Ammonium bicarbonate) to give (3-cyclopropyl-1-(9-ethyl-6-morpholino-8-(pyridin-4-yl)-9H-purin-2-yl)-1H-pyrazol-5-yl)methanol (19.4 mg, 0.043 mmol, 36%) as a white solid. 1H NMR (400 MHz, DMSO-d₆) δ 8.80 (dd, J=4.6, 1.5 Hz, 2H), 7.84 (dd, J=4.5, 1.6 Hz, 2H), 6.08 (s, 1H), 5.11 (t, J=5.8 Hz, 1H), 4.77-3.85 (m, 8H), 3.84-3.71 (m, 4H), 2.54 (dd, J=8.5, 5.3 Hz, 1H), 1.33 (t, J=7.2 Hz, 3H), 1.01-0.88 (m, 2H), 0.74-0.63 (m, 2H); LCMS, [M+H]+=446.9.

### Preparation of (1-(9-ethyl-6-morpholino-8-(pyridin-4-yl)-9H-purin-2-yl)-3-phenyl-1H-pyrazol-5-yl) methanol (Compound 223), ethyl 1-(9-ethyl-6-morpholino-8-(pyridin-4-yl)-9H-purin-2-yl)-3-phenyl-1H-pyrazole-5-carboxylate (Compound 224), (1-(9-ethyl-6-morpholino-8-(pyridin-4-yl)-9H-purin-2-yl)-5-phenyl-1H-pyrazol-3-yl)methanol (Compound 225) and 1-(9-ethyl-6-morpholino-8-(pyridin-4-yl)-9H-purin-2-yl)-3-phenyl-1H-pyrazole-5-carboxylic acid (Compound 226)

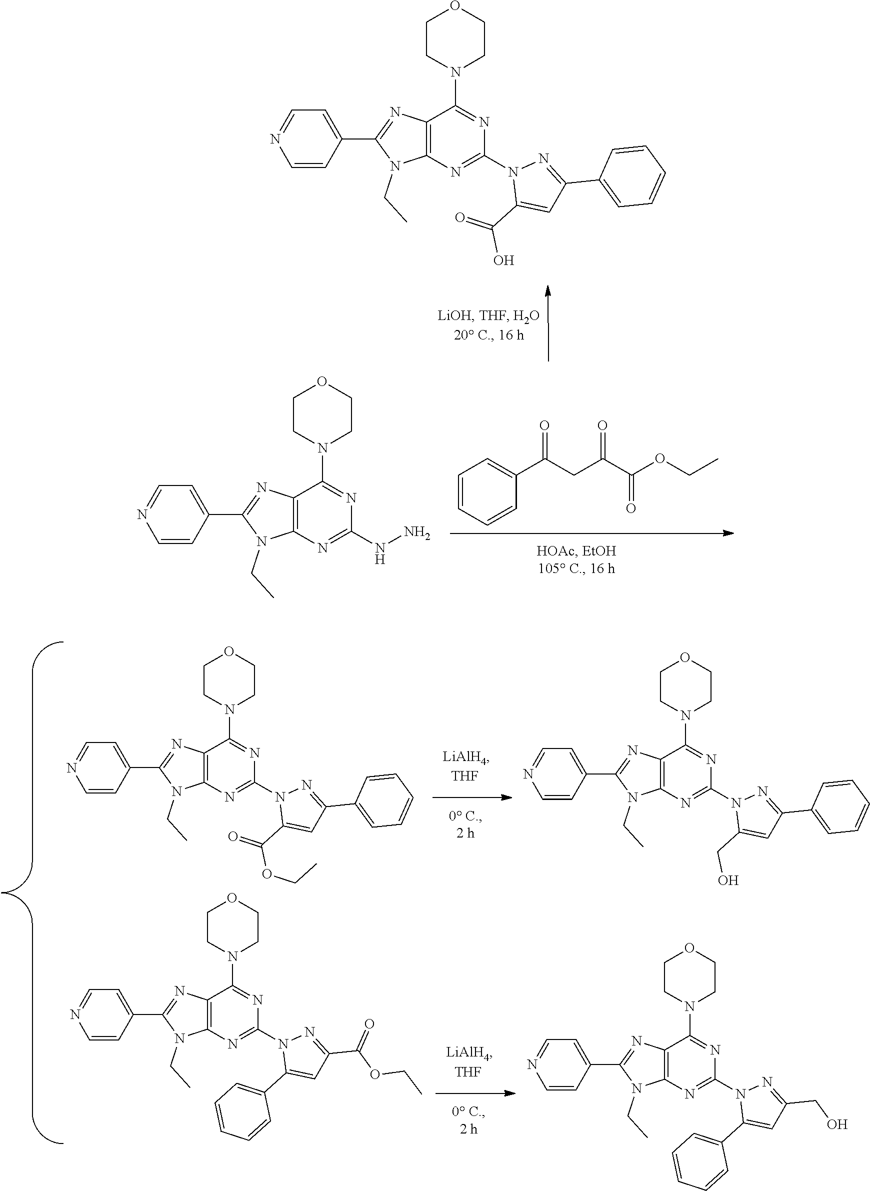

### Step 1: Synthesis of ethyl 1-(9-ethyl-6-morpholino-8-(pyridin-4-yl)-9H-purin-2-yl)-5-phenyl-1H-pyrazole-3-carboxylate (Compound 224) and ethyl 1-(9-ethyl-6-morpholino-8-(pyridin-4-yl)-9H-purin-2-yl)-3-phenyl-1H-pyrazole-5-carboxylate

A solution of 4-(9-ethyl-2-hydrazinyl-8-(pyridin-4-yl)-9H-purin-6-yl)morpholine (200 mg, 0.5 mmol) and ethyl 2,4-dioxo-4-phenylbutanoate (220 mg, 1.0 mmol) in acetic acid (5 mL) and ethanol (5 mL) was stirred at 105° C. for 16 h under argon. The mixture was concentrated and the residue was purified by pre-HPLC (BOSTON pHlex ODS 10 μm 21.2×250 mm 120 A. The mobile phase was acetonitrile/0.1% Ammonium bicarbonate) to get ethyl 1-(9-ethyl-6-morpholino-8-(pyridin-4-yl)-9H-purin-2-yl)-3-phenyl-1H-pyrazole-5-carboxylate (50 mg, 16%) and ethyl 1-(9-ethyl-6-morpholino-8-(pyridin-4-yl)-9H-purin-2-yl)-5-phenyl-1H-pyrazole-3-carboxylate (8.1 mg, 2.6%) as white solids.

Compound 224: ¹H NMR (400 MHz, DMSO d₆) δ 8.80 (d, J=5.8 Hz, 2H), 7.82 (d, J=5.9 Hz, 2H), 7.39 (dd, J=7.4, 3.6 Hz, 3H), 7.30 (dd, J=6.9, 2.4 Hz, 2H), 7.08 (s, 1H), 4.5 (bs, 4H), 4.36 (q, J=7.2 Hz, 2H), 4.29 (q, J=7.2 Hz, 2H), 3.53 (bs, 4H), 1.35 (t, J=7.1 Hz, 3H), 1.19 (t, J=7.2 Hz, 3H). LCMS: (ESI) m/z: 524.8 [M+H]⁺.

### Step 2: Synthesis of (1-(9-ethyl-6-morpholino-8-(pyridin-4-yl)-9H-purin-2-yl)-3-phenyl-1H-pyrazol-5-yl)methanol (Compound 223)

To a solution of Ethyl 1-(9-ethyl-6-morpholino-8-(pyridin-4-yl)-9H-purin-2-yl)-3-phenyl-1H-pyrazole-5-carboxylate (52 mg, 0.1 mmol) in anhydrous tetrahydrofuran (5 mL) was added a solution of lithium aluminum hydride (0.5 mL, 0.5 mmol, 1 M in THF) at 0° C. The mixture was stirred under nitrogen for 2 h and was quenched by the careful addition of sodium sulfate decahydrate with ice-bath cooling. The mixture was diluted with tetrahydrofuran (50 mL), stirred for 15 min, filtered and washed with tetrahydrofuran (50 mL). The combined filtrates were evaporated in vacuo. The residue was purified by pre-HPLC (BOSTON pHlex ODS 10 μm 21.2×250 mm 120 A. The mobile phase was acetonitrile/0.1% Ammonium bicarbonate) to give (1-(9-ethyl-6-morpholino-8-(pyridin-4-yl)-9H-purin-2-yl)-3-phenyl-1H-pyrazol-5-yl)methanol (23.2 mg, 32%) as a white solid.

¹H NMR (400 MHz, DMSO d₆) δ 8.79 (dd, J=4.5, 1.6 Hz, 2H), 7.81 (dd, J=4.5, 1.6 Hz, 2H), 7.40-7.31 (m, 3H), 7.27-7.22 (m, 2H), 6.56 (s, 1H), 4.54 (s, 2H), 4.26 (d, J=7.3 Hz, 2H), 3.47 (s, 4H), 3.33-3.24 (m, 4H), 1.19 (t, J=7.2 Hz, 3H); LCMS: (ESI) m/z: 483.1 [M+H]⁺.

### Step 4: Synthesis of (1-(9-ethyl-6-morpholino-8-(pyridin-4-yl)-9H-purin-2-yl)-5-phenyl-1H-pyrazol-3-yl)methanol (Compound 225)

To a solution of Ethyl 1-(9-ethyl-6-morpholino-8-(pyridin-4-yl)-9H-purin-2-yl)-5-phenyl-1H-pyrazole-3-carboxylate (29 mg, 0.055 mmol) in anhydrous tetrahydrofuran (5 mL) was added a solution of lithium aluminum hydride (0.5 mL, 0.5 mmol, 1 M in THF) at 0° C. and stirred under nitrogen for 2 h. The reaction was quenched by careful addition of sodium sulfate decahydrate with ice bath cooling. The mixture was diluted with tetrahydrofuran (50 mL), stirred for 15 min, filtered and washed with tetrahydrofuran (50 mL). The combined filtrates were evaporated in vacuo. The residue was purified by pre-HPLC (BOSTON pHlex ODS 10 μm 21.2×250 mm 120 A. The mobile phase was acetonitrile/0.1% Ammonium bicarbonate) to give (1-(9-ethyl-6-morpholino-8-(pyridin-4-yl)-9H-purin-2-yl)-5-phenyl-1H-pyrazol-3-yl)methanol (17.5 mg, 58%) as a white solid.

¹H NMR (400 MHz, DMSO-d₆) δ 8.81 (dd, J=4.5, 1.6 Hz, 2H), 7.92 (d, J=6.8 Hz, 2H), 7.85 (dd, J=4.5, 1.6 Hz, 2H), 7.47 (t, J=7.5 Hz, 2H), 7.37 (t, J=7.3 Hz, 1H), 6.95 (s, 1H), 5.47 (t, J=5.9 Hz, 1H), 5.00 (d, J=5.7 Hz, 2H), 4.91-3.85 (m, 6H), 3.84-3.77 (m, 4H), 1.38 (t, J=7.2 Hz, 3H); LCMS: (ESI) m/z: 482.8 [M+H]⁺.

### Step 4: Synthesis of 1-(9-ethyl-6-morpholino-8-(pyridin-4-yl)-9H-purin-2-yl)-3-phenyl-1H-pyrazole-5-carboxylic acid (Compound 226)

To a mixture of ethyl 1-(9-ethyl-6-morpholino-8-(pyridin-4-yl)-9H-purin-2-yl)-3-phenyl-1H-pyrazole-5-carboxylate (60 mg, 0.114 mmol) in tetrahydrofuran (3 mL) and water (3 mL) was added lithium hydroxide (5.5 gL, 0.229 mmol) at 20° C. The mixture was stirred at room temperature for 16 h. The reaction mixture was purified by pre-HPLC (BOSTON pHlex ODS 10 μm 21.2×250 mm 120 A. The mobile phase was acetonitrile/0.1% Ammonium bicarbonate) to give 1-(9-ethyl-6-morpholino-8-(pyridin-4-yl)-9H-purin-2-yl)-3-phenyl-1H-pyrazole-5-carboxylic acid (40.8 mg, 66%) as a white solid.

¹H NMR (400 MHz, DMSO-d₆) δ 8.86-8.74 (m, 2H), 7.82 (dd, J=4.5, 1.6 Hz, 2H), 7.41-7.33 (m, 3H), 7.32-7.26 (m, 2H), 6.97 (s, 1H), 4.90-4.01 (m, 4H), 3.51 (s, 6H), 1.20 (t, J=7.2 Hz, 3H); LCMS: (ESI) m/z: 496.8 [M+H]⁺.

### Synthesis of (1-(9-ethyl-6-morpholino-8-(pyridin-4-yl)-9H-purin-2-yl)-4-phenyl-1H-pyrazol-5-yl)methanol (Compound 227)

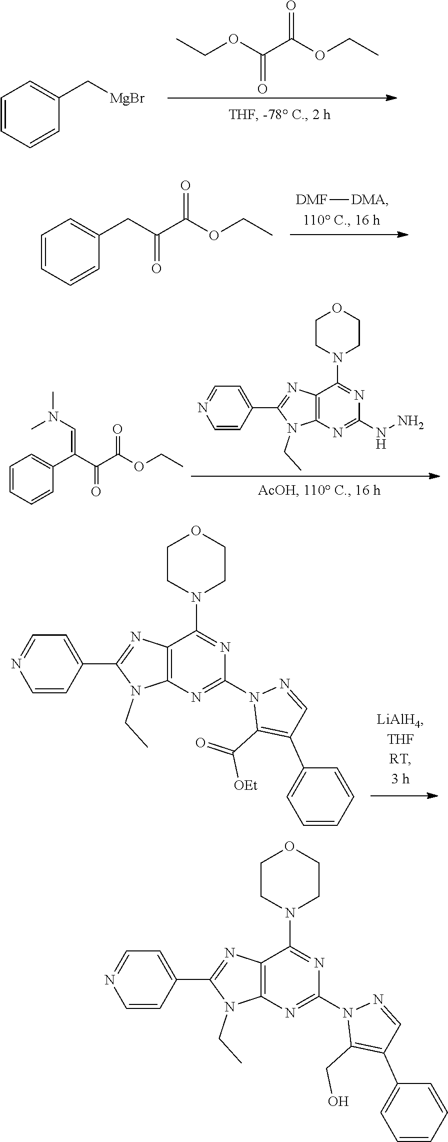

### Step 1: Preparation of ethyl 2-oxo-3-phenylpropanoate

To a mixture of diethyl oxalate (300 mg, 2 mmol) in THE (20 mL) was added a solution of benzylmagnesium bromide (2 mL, 2 mmol) dropwise under −78° C., and the resulting mixture was stirred at room temperature for 2 h. The mixture was filtered and the filtrate was concentrated. The residue was purified by flash chromatography (petroleum ether/ethyl acetate=2/1) to obtain the product as yellow oil (270 mg, 70.2%). LCMS (ESI) m/z: 192.9 [M+H]+.

### Step 2: Preparation of ethyl (E)-4-(dimethylamino)-2-oxo-3-phenylbut-3-enoate

A mixture of (ethyl (E)-4-(dimethylamino)-2-oxo-3-phenylbut-3-enoate (270 mg, 1.42 mmol) in N,N-dimethylformamide dimethyl acetal (10 mL) was stirred at 110° C. for 16 h. The mixture was concentrated and purified by flash chromatography (petroleum ether/ethyl acetate=2/1) to afford the product as a yellow oil (330 mg, 94%).

### Step 3: Preparation of ethyl 1-(9-ethyl-6-morpholino-8-(pyridin-4-yl)-9H-purin-2-yl)-4-phenyl-1H-pyrazole-5-carboxylate

A mixture of 4-(9-ethyl-2-hydrazineyl-8-(pyridin-4-yl)-9H-purin-6-yl)morpholine (340 mg, 1 mmol) and ethyl (E)-4-(dimethylamino)-2-oxo-3-phenylbut-3-enoate (247 mg, 1 mmol) in acetic acid (10 mL) was stirred at 110° C. for 16 h. The mixture was concentrated and purified by flash chromatography (petroleum ether/ethyl acetate=1/1) to give the desired product as a yellow oil. (260 mg, 50%)

### Step 4: Preparation of (1-(9-ethyl-6-morpholino-8-(pyridin-4-yl)-9H-purin-2-yl)-4-phenyl-1H-pyrazol-5-yl)methanol

To a mixture of ethyl 1-(9-ethyl-6-morpholino-8-(pyridin-4-yl)-9H-purin-2-yl)-4-phenyl-1H-pyrazole-5-carboxylate (80 mg, 0.157 mmol) in tetrahydrofuran (10 mL) was added lithium aluminum hydride (12 mg, 0.314 mmol) potion-wise, and the resulting mixture was stirred at room temperature for 2 h. The reaction was quenched by the careful addition of sodium sulfate decahydrate with ice bath cooling. Tetrahydrofuran (50 mL) was added to the reaction mixture, and stirring was continued for 15 min, the mixture was filtered and the solid was washed with tetrahydrofuran (50 mL). The combined filtrates were evaporated in vacuo. The residue was purified by prep-HPLC (SunFire C18, 4.6*50 mm, 3.5 um column Xbridge C18 3.5 μm 4.6×50 mm column. The mobile phase was acetonitrile/10 mM Formic acid aqueous solution.) to obtain the desired product as yellow solid (0.0112 g, 13%) ¹H NMR (400 MHz, DMSO-d₆) δ 8.81 (dd, J=4.5, 1.6 Hz, 2H), 7.93 (s, 1H), 7.85 (dd, J=4.5, 1.6 Hz, 2H), 7.59 (d, J=7.1 Hz, 2H), 7.48 (t, J=7.7 Hz, 2H), 7.37 (d, J=7.4 Hz, 1H), 5.16 (s, 1H), 4.89 (d, J=5.6 Hz, 2H), 4.45-4.05 (m, 6H), 3.79 (d, J=4.4 Hz, 4H), 1.37 (t, J=7.2 Hz, 3H); LCMS (ESI) m/z: 482.8 [M+H]+.

### Synthesis of (1-(9-ethyl-6-morpholino-8-(pyridin-4-yl)-9H-purin-2-yl)-4-phenyl-1H-pyrazol-3-yl)methanol (Compound 228)

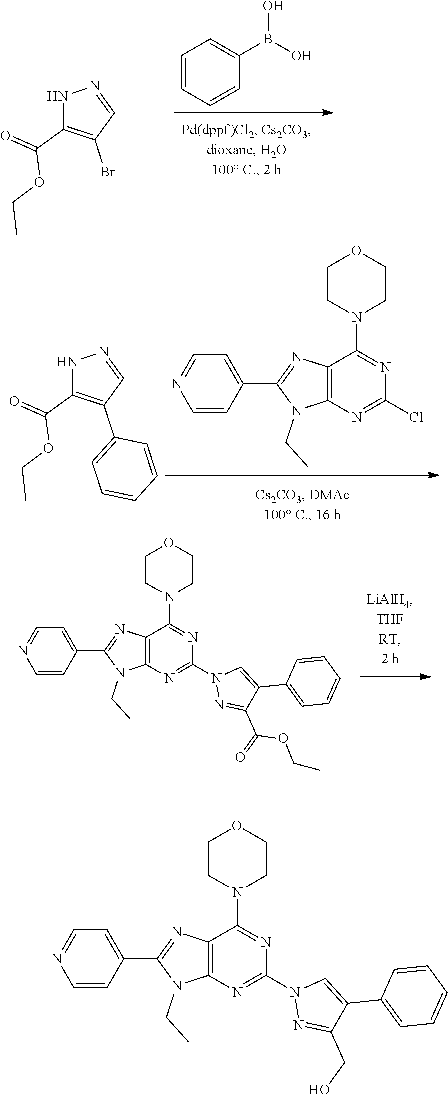 [SMILES: CCOC(=O)c1[nH]ncc1Br ; OB(O)c1ccccc1 ; CCOC(=O)c1[nH]ncc1-c1ccccc1 ; CCn1c(-c2ccncc2)nc2c(N3CCOCC3)nc(Cl)nc21 ; CCOC(=O)c1nn(-c2nc(N3CCOCC3)c3nc(-c4ccncc4)n(CC)c3n2)cc1-c1ccccc1 ; CCn1c(-c2ccncc2)nc2c(N3CCOCC3)nc(-n3cc(-c4ccccc4)c(CO)n3)nc21]

### Step 1: Preparation of ethyl 4-phenyl-1H-pyrazole-5-carboxylate

To a solution of ethyl 4-bromo-1H-pyrazole-5-carboxylate (300 mg, 1.37 mmol) in dioxane/water (20 mL/2 mL) were added phenylboronic acid (218 mg, 1.79 mmol), cesium carbonate (1.345 g, 4.11 mmol) and bis(diphenylphosphino)ferrocene]dichloropalladium(II) (100 mg, 0.137 mmol) at 25° C., and the reaction mixture was stirred at 100° C. for 2 h under argon protection. The resultant mixture was filtered and the filtrate was concentrated. The residue was purified by flash chromatography (petroleum ether/ethyl acetate=1/1) to give the product as a yellow oil (350 mg, 90.2%). LCMS (ESI) m/z: 216.9 [M+H]+.

### Step 2: Preparation of ethyl 1-(9-ethyl-6-morpholino-8-(pyridin-4-yl)-9H-purin-2-yl)-4-phenyl-1H-pyrazole-3-carboxylate

To a mixture of ethyl 4-phenyl-1H-pyrazole-5-carboxylate (108 mg, 0.5 mmol) and cesium carbonate (325 mg, 1 mmol) in N,N-dimethylaniline (10 mL) was added 4-(2-chloro-9-ethyl-8-(pyridin-4-yl)-9H-purin-6-yl)morpholine (172 mg, 0.5 mmol) and the resulting mixture was stirred at 100° C. for 16 h. The mixture was concentrated and purified by flash chromatography (petroleum ether/ethyl acetate=2/1) to give the product as a yellow oil (90 mg, 35%).

### Step 3: Preparation of (1-(9-ethyl-6-morpholino-8-(pyridin-4-yl)-9H-purin-2-yl)-4-phenyl-1H-pyrazol-3-yl)methanol

To a mixture of (9-ethyl-6-morpholino-8-(pyridin-4-yl)-9H-purin-2-yl)-4-phenyl-1H-pyrazole-3-carboxylate (80 mg, 0.157 mmol) in tetrahydrofuran (10 mL) was added lithium aluminum hydride (12 mg, 0.314 mmol), and the resulting mixture was stirred at room temperature for 2 h. The reaction was quenched by careful addition of sodium sulfate decahydrate with ice bath cooling. Tetrahydrofuran (50 mL) was added to the reaction mixture, and stirring was continued for 15 min, the mixture was filtered and the solids were washed with tetrahydrofuran (50 mL). The combined filtrates were evaporated in vacuo and the residue was purified by prep-HPLC (SunFire C18, 4.6*50 mm, 3.5 um column Xbridge C18 3.5 μm 4.6×50 mm column. The mobile phase was acetonitrile/10 mM Formic acid aqueous solution) to give the desired product as yellow solid (0.0173 g, 23%).

¹H NMR (500 MHz, DMSO-d₆) δ 8.94 (s, 1H), 8.80 (d, J=5.7 Hz, 2H), 7.85 (d, J=6.7 Hz, 2H), 7.80 (d, J=6.7 Hz, 4H), 7.45 (t, J=7.6 Hz, 2H), 7.32 (t, J=7.3 Hz, 1H), 6.05 (s, 1H), 4.75-4.06 (m, 8H), 3.80 (d, J=4.2 Hz, 4H), 1.34 (t, J=7.1 Hz, 3H). LCMS (ESI) m/z: 482.9 [M+].

### Synthesis of 2-(1-(9-ethyl-6-morpholino-8-(pyridin-4-yl)-9H-purin-2-yl)-5-phenyl-1H-pyrazol-3-yl)ethanol (Compound 229)

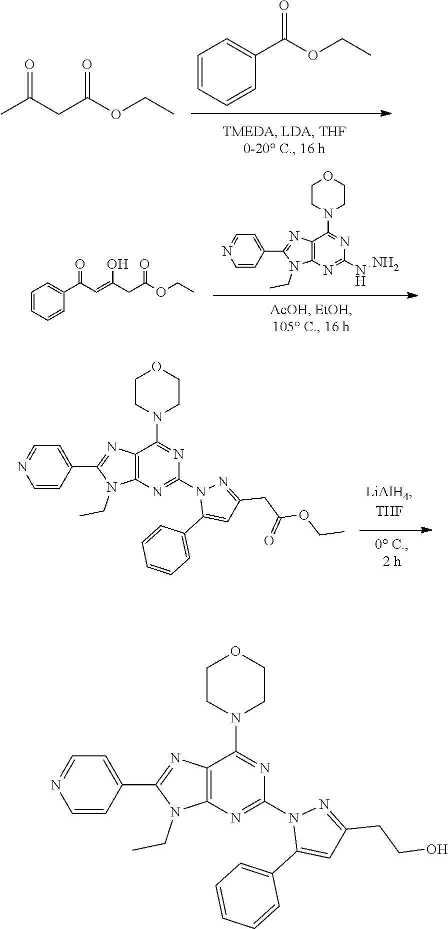

### Step 1: Synthesis of (Z)-ethyl 3-hydroxy-5-oxo-5-phenylpent-3-enoate

To a solution of lithium diisopropylamide (12.5 mL, 25 mmol) in tetrahydrofuran (20 mL) at 0° C. was added a solution of tetramethylethylenediamine (1 mL, 11 mmol) in tetrahydrofuran (25 mL). Then a mixture of ethyl acetoacetate (1.3 g, 10 mmol) and ethyl benzoate (1.875 g, 12.5 mmol) in tetrahydrofuran (10 mL) was added to the reaction mixture under an inert atmosphere. After stirring at room temperature for 16 h, the reaction was quenched with a solution of acetic acid (3 g, 50 mmol) in tetrahydrofuran (40 mL), and then treated with water (25 mL) at 0° C. The organic layer was separated, dried over sodium sulfate and concentrated. The residue was purified by silica gel chromatography (petroleum ether-ethyl acetate 70:10) to obtain 3-hydroxy-5-oxo-5-phenyl-pent-3-enoic acid ethyl ester as yellow oil (1.1 g, 47%). LCMS: [M+H]⁺=235.2.

### Step 2: Synthesis of ethyl 2-(1-(9-ethyl-6-morpholino-8-(pyridin-4-yl)-9H-purin-2-yl)-5-phenyl-1H-pyrazol-3-yl)acetate

A solution of 4-(9-ethyl-2-hydrazinyl-8-(pyridin-4-yl)-9H-purin-6-yl)morpholine (102 mg, 0.3 mmol) and (Z)-ethyl 3-hydroxy-5-oxo-5-phenylpent-3-enoate (105 mg, 0.45 mmol) in acetic acid (2 mL) and ethanol (2 mL) was stirred at 105° C. for 16 h under nitrogen. The reaction mixture was concentrated and purified by pre-HPLC (BOSTON pHlex ODS 10 μm 21.2×250 mm 120 A. The mobile phase was acetonitrile/0.1% Ammonium bicarbonate) to obtain ethyl 2-(1-(9-ethyl-6-morpholino-8-(pyridin-4-yl)-9H-purin-2-yl)-5-phenyl-1H-pyrazol-3-yl)acetate (114 mg, 71%) as white solid. LCMS: [M+H]⁺=539.0.

### Step 3: Synthesis of 2-(1-(9-ethyl-6-morpholino-8-(pyridin-4-yl)-9H-purin-2-yl)-5-phenyl-1H-pyrazol-3-yl)ethanol

To a solution of ethyl 2-(1-(9-ethyl-6-morpholino-8-(pyridin-4-yl)-9H-purin-2-yl)-5-phenyl-1H-pyrazol-3-yl)acetate (87 mg, 0.16 mmol) in anhydrous tetrahydrofuran (5 mL) was added a solution of lithium aluminum hydride (1 M in THF, 0.32 mL, 0.32 mmol,) at 0° C. The mixture was stirred under nitrogen atmosphere for 2 h. The reaction mixture was quenched by the carefully addition of sodium sulfate decahydrate with ice bath cooling. Tetrahydrofuran (50 mL) was added to the reaction mixture and the resultant mixture was stirred for 15 min and filtered. The filtrates were evaporated in vacuo and the residue was purified by pre-HPLC (BOSTON pHlex ODS 10 μm 21.2×250 mm 120 A. The mobile phase was acetonitrile/0.1% Ammonium bicarbonate) to give 2-(1-(9-ethyl-6-morpholino-8-(pyridin-4-yl)-9H-purin-2-yl)-5-phenyl-1H-pyrazol-3-yl)ethanol (16.6 mg, 0.0334 mmol, 16%) as a white solid.

¹H NMR (400 MHz, CDCl3) δ 8.78 (dd, J=4.5, 1.6 Hz, 2H), 7.65 (dd, J=4.5, 1.6 Hz, 2H), 7.35-7.29 (m, 5H), 6.38 (s, 1H), 4.5 (bs, 2H), 4.37 (q, J=7.2 Hz, 2H), 4.04 (d, J=5.0 Hz, 2H), 3.59 (bs, 6H), 3.04 (t, J=6.0 Hz, 2H), 2.87 (s, 1H), 1.32 (t, J=7.2 Hz, 3H); LCMS: [M+H]⁺=497.3.

### Synthesis of 1-(1-(9-ethyl-6-morpholino-8-(pyridin-4-yl)-9H-purin-2-yl)-5-phenyl-1H-pyrazol-3-yl)ethanol (Compound 230)

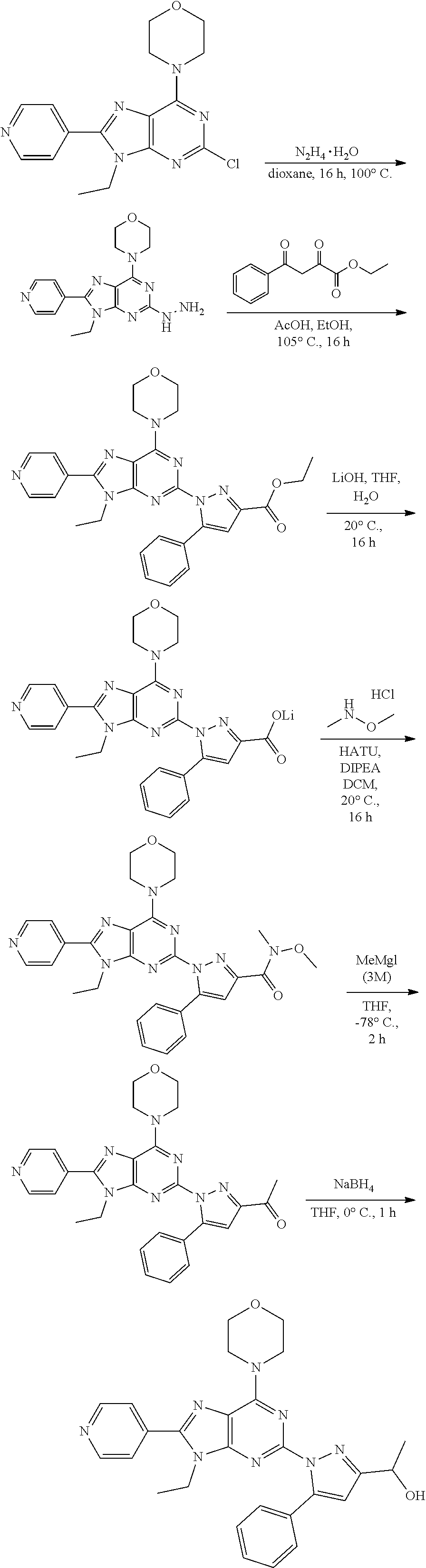

### Step 1: Synthesis of 4-(9-ethyl-2-hydrazinyl-8-(pyridin-4-yl)-9H-purin-6-yl)morpholine

A mixture of 4-(2-chloro-9-ethyl-8-(pyridin-4-yl)-9H-purin-6-yl)morpholine (345 mg, 1 mmol) and hydrazine hydrate (1 mL) in dioxane (6 mL) was stirred at 100° C. for 16 h. The reaction mixture was concentrated to give 4-(9-ethyl-2-hydrazinyl-8-(pyridin-4-yl)-9H-purin-6-yl)morpholine as yellow solid (340 mg, crude). LCMS: (ESI) m/z: 341.2 [M+H]⁺.

### Step 2: Synthesis of ethyl 1-(9-ethyl-6-morpholino-8-(pyridin-4-yl)-9H-purin-2-yl)-5-phenyl-1H-pyrazole-3-carboxylate

A solution of 4-(9-ethyl-2-hydrazinyl-8-(pyridin-4-yl)-9H-purin-6-yl)morpholine (276 mg, 0.8 mmol) and ethyl 2,4-dioxo-4-phenylbutanoate (352 mg, 1.6 mmol) in acetic acid (5 mL) and ethanol (5 mL) was stirred at 105° C. for 16 h under argon. The mixture was concentrated and the residue was purified by prep-HPLC (BOSTON pHlex ODS 10 μm 21.2×250 mm 120 A. The mobile phase was acetonitrile/0.1% Ammonium bicarbonate) to obtain ethyl 1-(9-ethyl-6-morpholino-8-(pyridin-4-yl)-9H-purin-2-yl)-5-phenyl-1H-pyrazole-3-carboxylate (230 mg, 55%) as white solid. LCMS: (ESI) m/z: 525.0 [M+H]⁺.

### Step 3: Synthesis of lithium 1-(9-ethyl-6-morpholino-8-(pyridin-4-yl)-9H-purin-2-yl)-5-phenyl-1H-pyrazole-3-carboxylate

A solution of ethyl 1-(9-ethyl-6-morpholino-8-(pyridin-4-yl)-9H-purin-2-yl)-5-phenyl-1H-pyrazole-3-carboxylate (314 mg, 0.6 mmol) and lithium hydroxide (29 mg, 1.2 mmol) in tetrahydrofuran (10 mL) and water (10 mL) was stirred at 20° C. for 16 h. The mixture was concentrated to get crude product 1-(9-ethyl-6-morpholino-8-(pyridin-4-yl)-9H-purin-2-yl)-5-phenyl-1H-pyrazole-3-carboxylic acid (360 mg, crude) as a white solid. LC-MS: (ESI) m/z: 497.4 [M+H]⁺.

### Step 4: Synthesis of 1-(9-ethyl-6-morpholino-8-(pyridin-4-yl)-9H-purin-2-yl)-N-methoxy-N-methyl-5-phenyl-1H-pyrazole-3-carboxamide

A solution lithium 1-(9-ethyl-6-morpholino-8-(pyridin-4-yl)-9H-purin-2-yl)-5-phenyl-1H-pyrazole-3-carboxylate (360 mg, 12.3 mmol), N,N-Diisopropylethylamine (310 mg, 2.4 mmol), N,O-dimethylhydroxylamine hydrochloride (65 mg, 0.66 mmol), and 2-(7-Azabenzotriazol-1-yl)-N,N,N′,N′-tetramethyluronium hexafluorophosphate (274 mg, 0.72 mmol) in N,N-dimethylformamide (20 mL) was stirred at 20° C. for 16 h. The resultant mixture was diluted with dichloromethane (100 mL) and washed with water (50 mL*3). The organic layer was dried over sodium sulfate and concentrated under reduced pressure to afford a residue, which was purified by flash chromatography (dichloromethane/methanol=10/1) to get 1-(9-ethyl-6-morpholino-8-(pyridin-4-yl)-9H-purin-2-yl)-N-methoxy-N-methyl-5-phenyl-1H-pyrazole-3-carboxamide as a white solid (260 mg, 74%). LCMS: (ESI) m/z: 540.2 [M+H]⁺.

### Step 5: Synthesis of 1-(1-(9-ethyl-6-morpholino-8-(pyridin-4-yl)-9H-purin-2-yl)-5-phenyl-1H-pyrazol-3-yl)ethenone

Methyl magnesium iodide (1 M) (0.4 mL, 0.4 mmol) was added to a solution of 1-(9-ethyl-6-morpholino-8-(pyridin-4-yl)-9H-purin-2-yl)-N-methoxy-N-methyl-5-phenyl-1H-pyrazole-3-carboxamide (108 mg, 0.2 mmol) in tetrahydrofuran (5 mL) at −78° C. After 2 h of stirring at −78° C. under argon, water (10 mL) was added the mixture carefully. The mixture was diluted with dichloromethane (100 mL) and washed with water (50 mL). The organic layer was dried over sodium sulfate and the solvent was removed under reduced pressure to afford a residue, which was purified by column chromatography (dichloromethane/methanol=10/1) to afford 1-(1-(9-ethyl-6-morpholino-8-(pyridin-4-yl)-9H-purin-2-yl)-5-phenyl-1H-pyrazol-3-yl)ethanone (76 mg, 61%) as yellow solid. LCMS: (ESI) m/z: 494.8 [M+]⁺.

### Step 6: Synthesis of 1-(1-(9-ethyl-6-morpholino-8-(pyridin-4-yl)-9H-purin-2-yl)-5-phenyl-1H-pyrazol-3-yl)ethanol

To a solution of 1-(1-(9-ethyl-6-morpholino-8-(pyridin-4-yl)-9H-purin-2-yl)-5-phenyl-1H-pyrazol-3-yl)ethanone (46 mg, 0.093 mmol) in anhydrous tetrahydrofuran (5 mL) was added sodium borohydride (18 mg, 0.465 mmol) at 0° C. and the mixture was stirred under nitrogen for 1 h. The reaction mixture was quenched by careful addition of HCl (1 M, 2 mL) at 0° C. and stirred for 15 min, The solvent were evaporated in vacuo and the residue was purified by pre-HPLC (BOSTON pHlex ODS 10 μm 21.2×250 mm 120 A. The mobile phase was acetonitrile/0.1% Ammonium bicarbonate) to afford 1-(1-(9-ethyl-6-morpholino-8-(pyridin-4-yl)-9H-purin-2-yl)-5-phenyl-1H-pyrazol-3-yl)ethanol (20.0 mg, 28%) as a white solid.

¹H NMR (500 MHz, DMSO) δ 8.78 (d, J=5.9 Hz, 2H), 7.80 (d, J=6.0 Hz, 2H), 7.37-7.30 (m, 3H), 7.25 (d, J=6.8 Hz, 2H), 6.56 (s, 1H), 5.26 (d, J=4.7 Hz, 1H), 4.86-4.79 (m, 1H), 4.27 (q, J=7.1 Hz, 4H), 3.51 (bs, 6H), 1.45 (d, J=6.5 Hz, 3H), 1.19 (t, J=7.2 Hz, 3H); LCMS: [M+]⁺ 496.8.

### Synthesis of (1-(9-ethyl-6-morpholino-8-(pyridin-4-yl)-9H-purin-2-yl)-1H-indazol-3-yl)methanol (Compound 231)

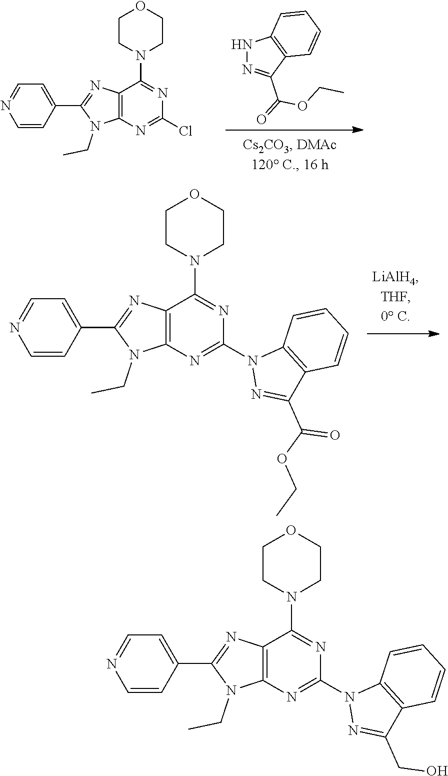

### Step 1: Synthesis of ethyl 1-(9-ethyl-6-morpholino-8-(pyridin-4-yl)-9H-purin-2-yl)-1H-indazole-3-carboxylate

A mixture of 4-(2-chloro-9-ethyl-8-(pyridin-4-yl)-9H-purin-6-yl)morpholine (690 mg, 2 mmol), ethyl 1H-indazole-3-carboxylate (456 mg, 2.4 mmol) and cesium carbonate (2 g, 6 mmol) in N,N-dimethylacetamide (5 mL) was stirred at 120° C. under argon atmosphere for 16 h. The mixture was filtered and concentrated to afford ethyl 1-(9-ethyl-6-morpholino-8-(pyridin-4-yl)-9H-purin-2-yl)-1H-indazole-3-carboxylate as a white solid (450 mg, crude). LCMS (ESI) m/z: 499.2 [M+H]+.

### Step 2: Synthesis of (1-(9-ethyl-6-morpholino-8-(pyridin-4-yl)-9H-purin-2-yl)-1H-indazol-3-yl)methanol

A mixture of ethyl 1-(9-ethyl-6-morpholino-8-(pyridin-4-yl)-9H-purin-2-yl)-1H-indazole-3-carboxylate (690 mg, 2 mmol) and lithium aluminum hydride (1 N in THF, 3.6 mL, 3.6 mmol) in tetrahydrofuran (10 mL) was stirred at 0° C. under argon atmosphere for 30 min and then at room temperature for 3 h. The reaction was quenched by the addition of glauber's salt and filtered. The filtrate was purified by prep-HPLC (Column Xbridge 21.2*250 mm C18, 10 um, Mobile Phase A: water (10 mmol/L ammonium bicarbonate) B: acetonitrile) to afford (1-(9-ethyl-6-morpholino-8-(pyridin-4-yl)-9H-purin-2-yl)-1H-indazol-3-yl)methanol as a white solid. (84.8 mg, 10%)

¹H NMR (400 MHz, DMSO-d₆) δ 8.81 (dd, J=4.5, 1.6 Hz, 2H), 8.66 (d, J=8.5 Hz, 1H), 7.98 (d, J=7.9 Hz, 1H), 7.88 (dd, J=4.6, 1.5 Hz, 2H), 7.63-7.55 (m, 1H), 7.33 (t, J=7.2 Hz, 1H), 5.96-5.04 (m, 1H), 4.90 (s, 2H), 4.47 (q, J=7.1 Hz, 6H), 4.35-4.10 (m, 4H), 3.82 (t, J=4 Hz, 4H), 1.41 (t, J=7.2 Hz, 3H); LCMS (ESI) m/z: 457.3 [M+H]+.

### Synthesis of 4-(9-methyl-2-(4-phenyl-2H-1,2,3-triazol-2-yl)-8-(pyridin-4-yl)-9H-purin-6-yl) morpholine (Compound 232) and 4-(9-methyl-2-(4-phenyl-1H-1,2,3-triazol-1-yl)-8-(pyridin-4-yl)-9H-purin-6-yl)morpholine (Compound 233)

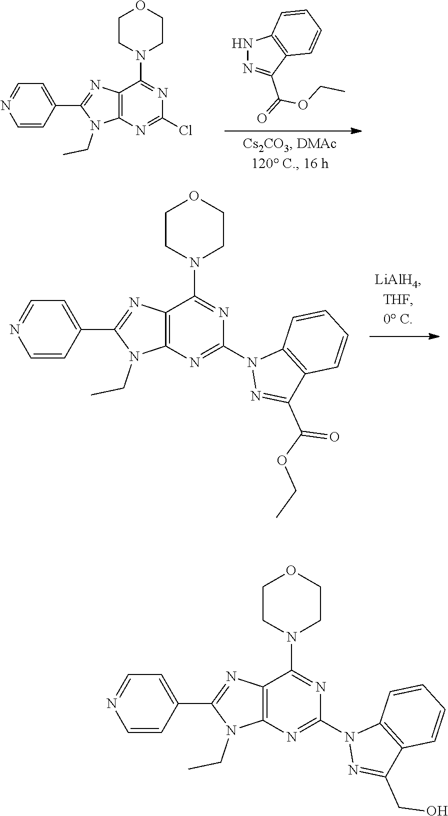 [SMILES: CCn1c(-c2ccncc2)nc2c(N3CCOCC3)nc(Cl)nc21 ; CCOC(=O)c1n[nH]c2ccccc12 ; CCOC(=O)c1nn(-c2nc(N3CCOCC3)c3nc(-c4ccncc4)n(CC)c3n2)c2ccccc12 ; CCn1c(-c2ccncc2)nc2c(N3CCOCC3)nc(-n3nc(CO)c4ccccc43)nc21]

A mixture of 4-(2-chloro-9-methyl-8-(pyridin-4-yl)-9H-purin-6-yl)morpholine (99 mg, 0.3 mmol), 4-phenyl-1 H-1,2,3-triazole (52 mg, 0.36 mmol) and cesium carbonate (293 mg, 0.9 mmol) in dry N,N-dimethylacetamide (5 mL) was stirred at 130° C. for 16 h. The crude products formed were purified by Prep-HPLC (BOSTON pHlex ODS 10 um 21 0.2×250 mm 120 A. The mobile phase was acetonitrile/0.1% formic acid) to get 4-(9-methyl-2-(4-phenyl-2H-1,2,3-triazol-2-yl)-8-(pyridin-4-yl)-9H-purin-6-yl)morpholine (Compound 232) (16.8 mg, 0.038 mmol, 5.5%) and 4-(9-methyl-2-(4-phenyl-1H-1,2,3-triazol-1-yl)-8-(pyridin-4-yl)-9H-purin-6-yl)morpholine (Compound 233) (30.0 mg, 0.068 mmol, 9.8%) as white solids. The regiochemistry assignments are arbitrary.

Compound 232: 1 H NMR (400 MHz, 00013) δ 8.82 (d, J=5.5 Hz, 2H), 8.21 (5, 1H), 7.99 (d, J=7.2 Hz, 2H), 7.76 (dd, J=4.7, 1.3 Hz, 2H), 7.49 (t, J=7.4 Hz, 2H), 7.42 (dd, J=8.4, 6.2 Hz, 1H), 4.84-4.27 (3, 4H), 4.07 (4, 3H), 3.96-63.83 (m, 4H); L3MS, [M+H]+ 439.8.

Compound 233: 1 H NMR (400 MHz, 00013) δ 8.83 (d, J=5.0 Hz, 2H), 8.74 (s, 1H), 8.02-7.94 (m, 2H), 7.80-7.73 (m, 2H), 7.47 (t, J=7.5 Hz, 2H), 7.40-7.36 (m, 1H), 5.11-4.09 (m, 4H), 4.05 (s, 3H), 3.95-3.87 (in, 4H); LHMS, [M+H]+ 440.3.

The following compounds were synthesized according to the protocol described above:

| Name | Structure | NMR, MS | # |
|---|---|---|---|
| 4-(9-ethyl-8- (pyridin-4-yl)-2- (1H-1,2,3- triazol-1-yl)-9H- purin-6- yl)morpholine | 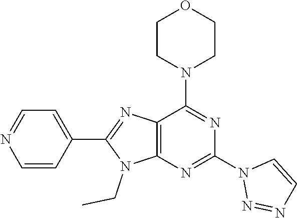 | ¹H NMR (400 MHz, DMSO-d₆) δ 8.94 (d, J = 1.2 Hz, 1H), 8.81 (dd, J = 4.5, 1.5Hz, 2H), 7.95 (d, J = 1.2 Hz, 1H), 7.86 (dd, J = 4.5, 1.6Hz, 2H), 4.80-4.10 (m, 6H), 3.80-3.79 (m, 4H), 1.36 (t, J = 7.2 Hz, 3H); LCMS (ESI) m/z: 414.1 [M + H]⁺. | 234 |
| 4-(9-ethyl-8- (pyridin-4-yl)-2- (2H-1,2,3- triazol-2-yl)-9H- purin-6- yl)morpholine | 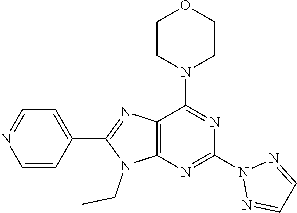 | ¹H NMR (400 MHz, DMSO-d₆) δ 8.81 (dd, J = 4.5, 1,6 Hz, 2H), 8.13 (s, 2H), 7.85 (dd, J = 4.5, 1.6 Hz, 2H), 4.75-4.05 (m, 6H), 3.78 (t, J = 4.8 Hz, 4H), 1.35 (t, J = 7.2 Hz, 3H); LCMS (ESI) m/z: 414.1 [M + H]⁺. | 235 |
| 4-(9-ethyl-2-(4- phenyl-2H- 1,2,3-triazol-2- yl)-8-(pyridin-4- yl)-9H-purin-6- yl)morpholine | 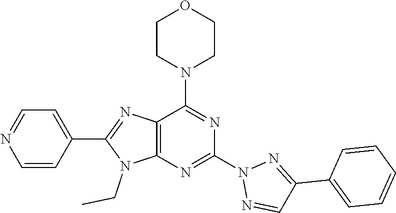 [SMILES: CCn1c(-c2ccncc2)nc2c(N3CCOCC3)nc(-n3ncc(-c4ccccc4)n3)nc21] | ¹H NMR (400 MHz, CDCl₃) δ 8.77 (s, 2H), 8.13 (s, 1H), 7.91 (d, J = 7.2 Hz, 2H), 7.65 (s, 2H), 7.42 (t, J = 8 Hz, 2H), 7.35 (t, J = 8 Hz, 1H), 4.65- 4.05 (m, 6H), 3.84 (s, 4H), 1.41 (t, J = 7.1 Hz, 3H). LCMS (ESI) m/z: 453.8 [M + H]⁺. | 236 |
| 4-(9-ethyl-2-(4- phenyl-1H- 1,2,3-triazol-1- yl)-8-(pyridin-4- yl)-9H-purin-6- yl)morpholine | 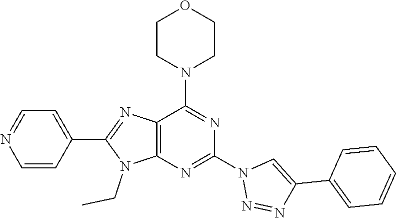 [SMILES: CCn1c(-c2ccncc2)nc2c(N3CCOCC3)nc(-n3cc(-c4ccccc4)nn3)nc21] | ¹H NMR (400 MHz, CDCl₃) δ 8.83 (d, J = 5.3 Hz, 2H), 8.74 (s, 1H), 7.98 (d, J = 7.4 Hz, 2H), 7.71 (d, J = 5.4 Hz, 2H), 7.47 (t, J = 7.4 Hz, 2H), 7.38 (d, J = 7.2 Hz, 1H), 4.60-4.01 (m, 6H), 3.91 (s, 4H), 1.51 (t, J = 7.1 Hz, 3H). LCMS (ESI) m/z: 453.8 [M + H]⁺. | 237 |
| 4-(9-ethyl-8- (pyridin-4-yl)-2- (4-m-tolyl-2H- 1,2,3-triazol-2- yl)-9H-purin-6- yl)morpholine | 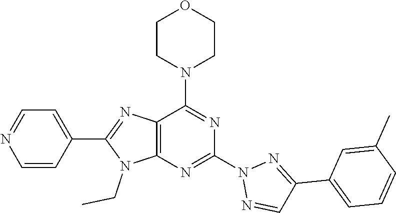 [SMILES: CCn1c(-c2ccncc2)nc2c(N3CCOCC3)nc(-n3ncc(-c4cccc(C)c4)n3)nc21] | ¹H NMR (400 MHz, CDCl₃) δ 8.82 (d, J = 4.8 Hz, 2H), 8.19 (s, 1H), 7.82 (s, 1H), 7.77 (d, J = 8 Hz, 1H), 7.72 (d, J = 8 Hz, 2H), 7.37 (t, J = 7.5 Hz, 1H), 7.23 (s, 1H), 4.75-4.30 (m, 6H), 3.92 (d, J = 4.3 Hz, 4H), 2.45 (s, 3H), 1.48 (t, J = 7.2 Hz, 3H); LCMS (ESI) m/z: 467.8 [M + H]⁺. | 238 |
| 4-(9-ethyl-8- (pyridin-4-yl)-2- (4-m-tolyl-1H- 1,2,3-triazol-1- yl)-9H-purin-6- yl)morpholine | 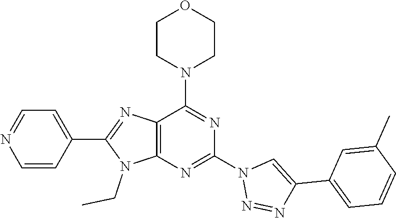 [SMILES: CCn1c(-c2ccncc2)nc2c(N3CCOCC3)nc(-n3cc(-c4cccc(C)c4)nn3)nc21] | ¹H NMR (400 MHz, CDCl₃) δ 8.83 (dd, J = 4.6, 1.4 Hz, 2H), 8.72 (s, 1H), 7.82 (s, 1H), 7.77- 7.68 (m, 3H), 7.35 (t, J = 7.6 Hz, 1H), 7.19 (d, J = 7.5 Hz, 1H), 4.70-4.05 (m, 6H), 3.97-3.85 (m, 4H), 2.44 (s, 3H), 1.51 (t, J = 7.2 Hz, 3H); LCMS (ESI) m/z: 467.8 [M + H]⁺. | 239 |
| 4-(9- cyclopropyl-8- (pyridin-4-yl)-2- (2H-1,2,3- triazol-2-yl)-9H- purin-6- yl)morpholine | 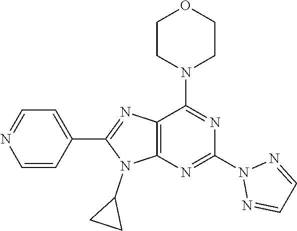 [SMILES: c1cc(-c2nc3c(N4CCOCC4)nc(-n4nccn4)nc3n2C2CC2)ccn1] | ¹H NMR (400 MHz, DMSO-d₆) δ 8.79 (dd, J = 4.5, 1.5 Hz, 2H), 8.13 (s, 2H), 8.01 (dd, J = 4.5, 1.6 Hz, 2H), 4.30 (bs, 4H), 3.75-3.45 (m, 5H), 1.15 (t, J = 6.5 Hz, 2H), 0.83 (s, 2H); LCMS (ESI) m/z: 390.0 [M + H]⁺. | 240 |
| 4-(9- cyclopropyl-8- (pyridin-4-yl)-2- (1H-1,2,3- triazol-1-yl)-9H- purin-6- yl)morpholine | 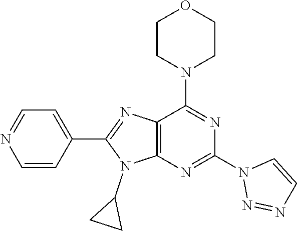 [SMILES: c1cc(-c2nc3c(N4CCOCC4)nc(-n4ccnn4)nc3n2C2CC2)ccn1] | ¹H NMR (400 MHz, DMSO-d₆) δ 8.89 (s, 1H), 8.80 (d, J = 8 Hz, 2H), 8.01 (d, J = 4 Hz, 2H), 7.95 (s, 1H), 4.58 (bs, 2H), 4.16 (bs, 2H), 3.82-3.78 (m, 5H), 1.16 (d, J = 5.7 Hz, 2H), 0.86 (s, 2H); LCMS (ESI) m/z: 390.0 [M + H]⁺. | 241 |

### Synthesis of 4-(9-methyl-2-(5-methyl-2H-benzo[d][1,2,3]triazol-2-yl)-8-(pyridin-4-yl)-9H-purin-6-yl)morpholine 2,2,2-trifluoroacetate (Compound 242) and a mixture of 4-(9-methyl-2-(5-methyl-1H-benzo[d][1,2,3]triazol-1-yl)-8-(pyridin-4-yl)-9H-purin-6-yl)morpholine 2,2,2-trifluoroacetate and 4-(9-methyl-2-(6-methyl-1H-benzo[d][1,2,3]triazol-1-yl)-8-(pyridin-4-yl)-9H-purin-6-yl)morpholine 2,2,2-trifluoroacetate (Compound 243)

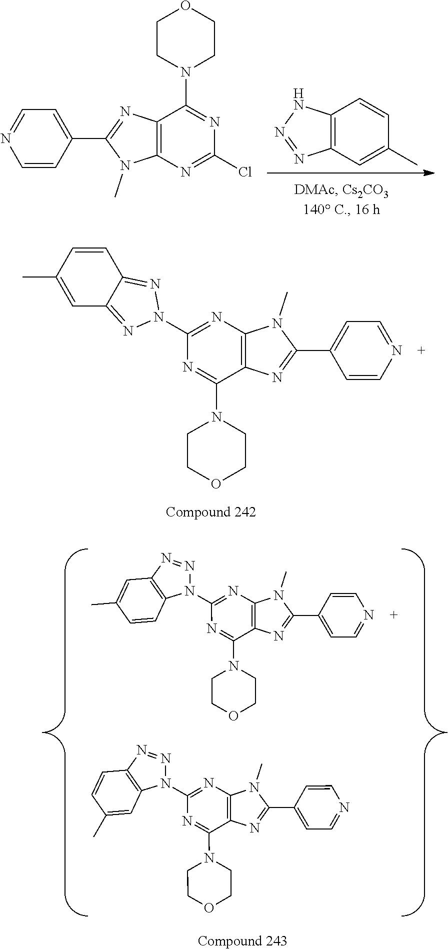

A mixture of 4-(2-chloro-9-methyl-8-(pyridin-4-yl)-9H-purin-6-yl)morpholine (132 mg, 0.4 mmol), 5-methyl-1H-benzo[d][1,2,3]triazole (64 mg, 0.48 mmol) and cesium carbonate (391 mg, 1.2 mmol) in dry N,N-dimethylacetamide (5 mL) was stirred at 140° C. for 16 h. The resultant products were purified by prep-HPLC (BOSTON pHlex ODS 10 μm 21.2×250 mm 120 A. The mobile phase was acetonitrile/0.1% trifluoroacetic acid) to get 4-(9-methyl-2-(5-methyl-2H-benzo[d][1,2,3]triazol-2-yl)-8-(pyridin-4-yl)-9H-purin-6-yl)morpholine (Compound 242) (7.3 mg, 0.0135 mmol, 2.7%) and a mixture of 4-(9-methyl-2-(5-methyl-1H-benzo[d][1,2,3]triazol-1-yl)-8-(pyridin-4-yl)-9H-purin-6-yl)morpholine and 4-(9-methyl-2-(6-methyl-1H-benzo[d][1,2,3]triazol-1-yl)-8-(pyridin-4-yl)-9H-purin-6-yl)morpholine (Compound 243) (139.7 mg, 0.258 mmol, 51.6%) as white solids. The regiochemistry assignments are arbitrary.

Compound 242: 1H NMR (400 MHz, DMSO-d₆) δ 8.84 (d, J=5.3 Hz, 2H), 8.02 (d, J=5.6 Hz, 2H), 7.95 (d, J=8.8 Hz, 1H), 7.80 (s, 1H), 7.38 (d, J=8.9 Hz, 1H), 4.30 (s, 4H), 4.00 (s, 3H), 3.82 (s, 4H), 2.50 (S, 3H); LCMS; P1: [M+H]+=427.8.

Compound 243: 1H NMR (400 MHz, DMSO-d₆) δ 8.86 (d, J=5.0 Hz, 2H), 8.51-8.23 (m, 1H), 8.12-7.88 (m, 3H), 7.55-7.37 (m, 1H), 4.41 (s, 4H), 4.03 (d, J=2.3 Hz, 3H), 3.84 (s, 4H), 2.54 (d, J=25.7 Hz, 3H); LCMS, [M+H]+=427.8.

### Synthesis of 4-(9-ethyl-2-(5-phenyl-1H-1,2,4-triazol-3-yl)-8-(pyridin-4-yl)-9H-purin-6-yl)morpholine (Compound 244)

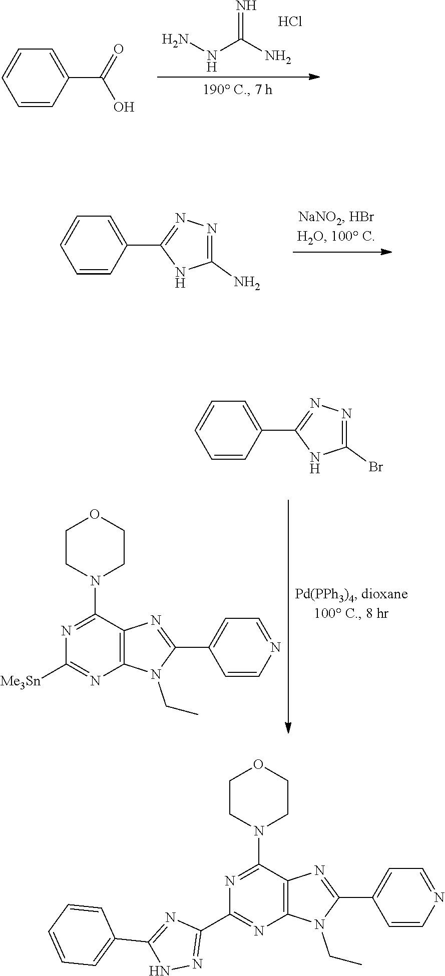

### Step 1: Preparation of 5-phenyl-4H-1,2,4-triazol-3-amine

A mixture of benzoic acid (1.2 g, 9.84 mmol) and hydrazinecarboximidamide hydrochloride (1.1 g, 9.84 mmol) was stirred at 190° C. for 7 h. Then the mixture was diluted with water (15 mL) and extracted with ethyl acetate (20 mL*3). The organic layer was combined, washed with brine (30 mL), dried over sodium sulfate, filtered and concentrated. The residue was purified by flash chromatography on silica gel (petroleum ether:ethyl acetate=75:25) to obtain the desired product as white solid. (1.5 g, 95.3%).

### Step 2: Preparation of 3-bromo-5-phenyl-4H-1,2,4-triazole

To a solution of 5-phenyl-4H-1,2,4-triazol-3-amine (750 mg, 4.69 mmol) in water (10 mL) was added sodium nitrite (638 mg, 9.38 mmol) at 0° C. and stirred at that temperature for 1 h. Then hydrogen bromide (10 mL) was added into the mixture and heated to 100° C. and stirred for 8 h. Then the mixture was diluted with water (15 mL) and extracted with ethyl acetate (20 mL*3). The organic layer was combined, washed with brine (30 mL), dried over sodium sulfate, filtered and concentrated. The residue was purified by flash chromatography on silica gel (petroleum ether:ethyl acetate=75:25) to give the desired product as white solid. (320 mg, 30.3%).

### Step 3: Preparation of 4-(9-ethyl-2-(5-phenyl-4H-1,2,4-triazol-3-yl)-8-(pyridin-4-yl)-9H-purin-6-yl)morpholine

To a solution of 4-(9-ethyl-8-(pyridin-4-yl)-2-(trimethylstannyl)-9H-purin-6-yl)morpholine (150 mg) in dioxane (10 mL) was added 3-bromo-5-phenyl-4H-1,2,4-triazole (110 mg, 0.493 mmol) and tetrakis(triphenylphosphine)palladium (58 mg, 0.05 mmol). The mixture was stirred at 100° C. for 16 h. The reaction mixture was concentrated, the residue was purified by Prep-HPLC (BOSTON pHlex ODS 10 μm 21.2×250 mm 120 A. The mobile phase was acetonitrile/0.1% Ammonium bicarbonate) to afford 4-(9-ethyl-2-(5-phenyl-4H-1,2,4-triazol-3-yl)-8-(pyridin-4-yl)-9H-purin-6-yl)morpholine (15.4 mg, 7.0%) as white solid. ¹H NMR (400 MHz, DMSO) δ 8.82 (d, J=6.0 Hz, 2H), 8.13 (d, J=7.1 Hz, 2H), 7.88 (d, J=6.0 Hz, 2H), 7.52 (t, J=8 Hz, 2H), 7.46 (t, J=8 Hz, 1H), 4.56-4.49 (m, 2H), 4.51-4.23 (m, 4H), 3.86-3.71 (m, 4H), 1.36 (t, J=7.2 Hz, 3H); LCMS (ESI) m/z: 453.8[M+H]+.

### Synthesis of N′-(4-morpholino-6-(3-phenyl-1H-pyrazol-1-yl)-1,3,5-triazin-2-yl)isonicotinohydrazide (Compound 245), 4-(5-(3-phenyl-1H-pyrazol-1-yl)-2-(pyridin-4-yl)-[1,2,4]triazolo[1,5-a][1,3,5]triazin-7-yl)morpholine (Compound 246), 4-(7-(3-phenyl-1H-pyrazol-1-yl)-2-(pyridin-4-yl)-[1,2,4]triazolo[1,5-a][1,3,5]triazin-5-yl)morpholine (Compound 247) and 4-(5-(3-phenyl-1H-pyrazol-1-yl)-3-(pyridin-4-yl)-[1,2,4]triazolo[4,3-a][1,3,5]triazin-7-yl)morpholine (Compound 248)

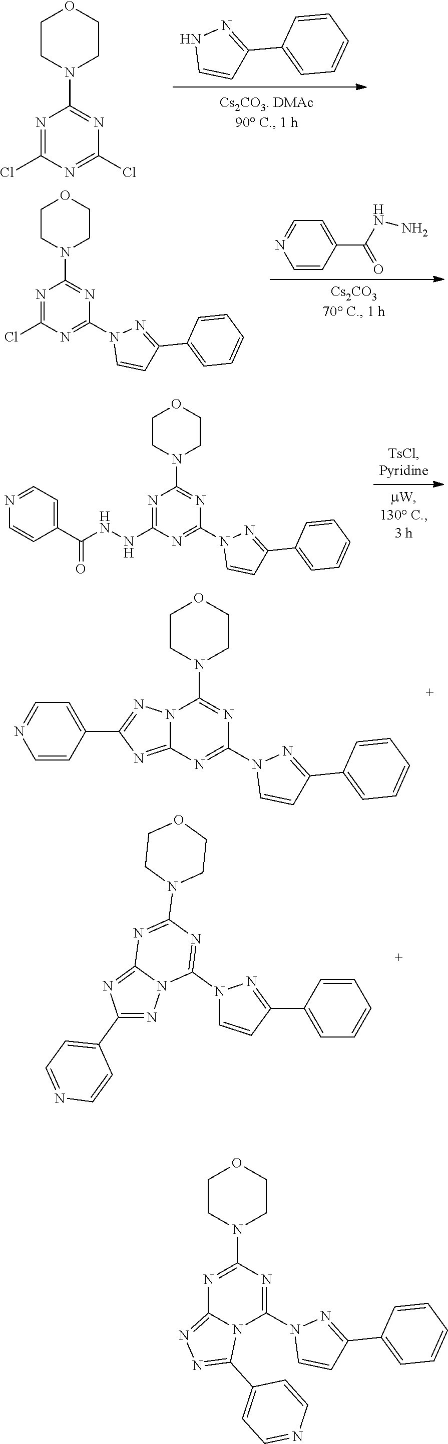

### Step 1: Synthesis of 4-(4-chloro-6-(3-phenyl-1H-pyrazol-1-yl)-1,3,5-triazin-2-yl)morpholine

A mixture of 4-(4,6-dichloro-1,3,5-triazin-2-yl)morpholine (470 mg, 2 mmol) and 3-phenyl-1H-pyrazole (288 mg, 2 mmol) and cesium carbonate (1.304 g, 4 mmol) in N,N-dimethylformamide (10 mL) was stirred at 90° C. for 1 h. The reaction mixture was directly used for the next step without further purification. LCMS: (ESI) m/z: 342.8 [M+H]⁺

### Step 2: Synthesis of N′-(4-morpholino-6-(3-phenyl-1H-pyrazol-1-yl)-1,3,5-triazin-2-yl)isonicotinohydrazide

The product from step-1 was mixed with isonicotinohydrazide (274 mg, 2 mmol) and cesium carbonate (652 mg, 2 mmol) and stirred at 70° C. for 1 h. The reaction mixture was purified by pre-HPLC (BOSTON pHlex ODS 10 μm 21.2×250 mm 120 A. The mobile phase was acetonitrile/0.1% Ammonium bicarbonate) to afford N′-(4-morpholino-6-(3-phenyl-1H-pyrazol-1-yl)-1,3,5-triazin-2-yl)isonicotinohydrazide (490 mg, 44%) as a yellow solid.

Compound 245: 1H NMR (400 MHz, DMSO-d₆) δ 10.83 (s, 1H), 9.86 (d, J=101.1 Hz, 1H), 8.95-8.50 (m, 3H), 8.08-7.71 (m, 4H), 7.59-7.34 (m, 3H), 7.08 (d, J=23.6 Hz, 1H), 3.95-3.53 (m, 8H); LCMS: (ESI) m/z: 443.9 [M+H]⁺

### Step 3: Synthesis of compounds 246, 247 and 248

A solution of N′-(4-morpholino-6-(3-phenyl-1H-pyrazol-1-yl)-1,3,5-triazin-2-yl)isonicotinohydrazide (130 mg, 0.3 mmol) and toluene-4-sulfonyl chloride (115 mg, 0.6 mmol) in pyridine (2 mL) was stirred at 130° C. under microwave for 3 h. The mixture was concentrated and the residue was purified by pre-HPLC (BOSTON pHlex ODS 10 um 21.2×250 mm 120 A. The mobile phase was acetonitrile/0.1% formic acid) to afford compound 247 (2 mg, 0.8%), compound 246 (8.9 mg, 3.7%), and compound 248 (29.1 mg, 12%) as white solids. Assignments for compounds 246 and 247 are arbitrary.

Compound 246: 1 H NMR (400 MHz, CDCl3) δ 8.79 (dd, J=4.5, 1.5 Hz, 2H), 8.68 (d, J=2.8 Hz, 1H), 8.11 (dd, J=4.6, 1.4 Hz, 2H), 7.97 (d, J=7.1 Hz, 2H), 7.45 (q, J=7.6 Hz, 2H), 7.40 (q, J=7.6 Hz, 1H), 6.86 (d, J=2.8 Hz, 1H), 4.96 (bs, 2H), 4.33 (bs, 2H), 4.04-3.94 (m, 4H); LCMS: (ESI) m/z: 425.9 [M+H]⁺

Compound 247: 1 H NMR (400 MHz, DMSO) δ 8.89 (d, J=2.8 Hz, 1H), 8.82 (dd, J=4.5, 1.5 Hz, 2H), 8.03 (d, J=7.1 Hz, 2H), 7.81 (dd, J=4.4, 1.6 Hz, 2H), 7.52 (t, J=7.4 Hz, 2H), 7.45 (d, J=7.3 Hz, 1H), 7.22 (d, J=2.8 Hz, 1H), 3.46-3.42 (m, 4H), 3.38-3.35 (m, 4H); LCMS: (ESI) m/z: 425.9 [M+H]⁺.

Compound 248: 1H NMR (400 MHz, CDCl3) δ 9.55 (d, J=3.0 Hz, 1H), 8.80 (d, J=6.0 Hz, 2H), 8.18 (dd, J=4.5, 1.5 Hz, 2H), 8.02 (dd, J=8.0, 1.3 Hz, 2H), 7.55-7.44 (m, 3H), 7.04 (d, J=3.0 Hz, 1H), 4.16 (bs, 2H), 4.06 (bs, 2H), 3.89-3.80 (m, 4H); LCMS: (ESI) m/z: 426.0 [M+H]⁺.

### Synthesis of 6,6-dimethyl-4-morpholino-2-(4-phenyl-1H-pyrazol-1-yl)-8,9-dihydro-6H-[1,4]oxazino[3,4-e]purine (Compound 249)

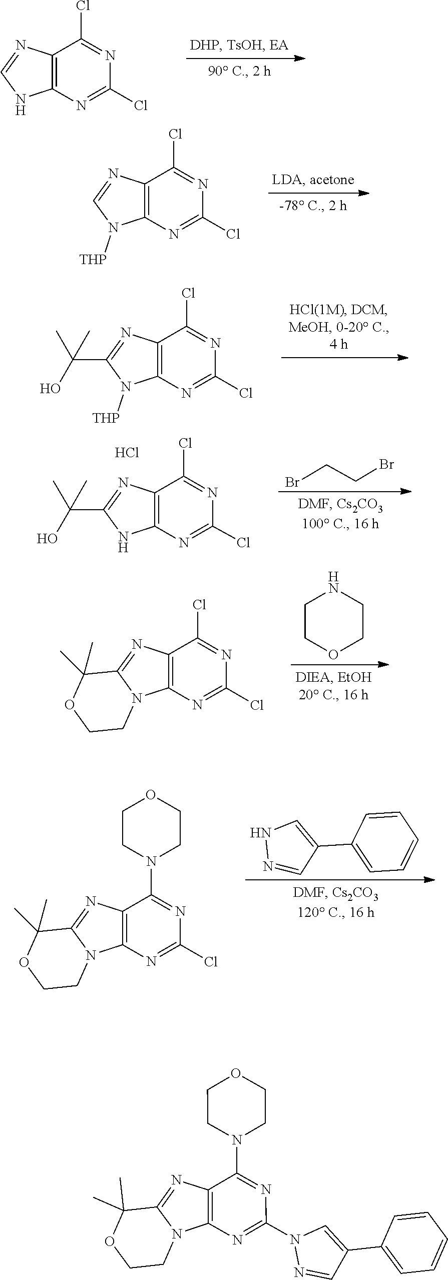

### Step 1: Synthesis of 2,6-dichloro-9-(tetrahydro-2H-pyran-2-yl)-9H-purine

A mixture of 2,6-dichloro-9H-purine (2.65 g, 14 mmol), 3,4-dihydro-2H-pyran (1.764 g, 21 mmol) and p-toluenesulfonic acid monohydrate (241 mg, 1.4 mmol) in ethyl acetate (25 mL) was heated at 90° C. for 2 h, cooled and concentrated in vacuo. The resulting residue was purified by column chromatography (SiO2, 0 to 10% ethyl acetate in petroleum) to obtain 2,6-dichloro-9-(tetrahydro-2H-pyran-2-yl)-9H-purine as yellow solid (4 g, 14.65 mmol, 98%). LCMS [M−84+H]+=189.0.

### Step 2: Synthesis of 2-(2,6-dichloro-9-(tetrahydro-2H-pyran-2-yl)-9H-purin-8-yl)propan-2-ol

Lithium diisopropylamide (19 mL, 19 mmol, 1 M in tetrahydrofuran) was added dropwise to a solution of 2,6-dichloro-9-(tetrahydro-2H-pyran-2-yl)-9H-purine (3.73 g, 13.66 mmol) in anhydrous THE (50 mL) at −78° C. The resulting solution was stirred at −78° C. for 30 min and then acetone (2 mL, 27.32 mmol) was added and the reaction mixture was stirred further at −78° C. for 90 min and at room temperature for 30 min. The reaction was quenched with water (20 mL) and the resultant mixture was extracted with ethyl acetate (100 mL*2). The combined organic extracts were dried with sodium sulfate and concentrated in vacuo. The resulting residue was purified by column chromatography (SiO2, 0 to 20% ethyl acetate in petroleum ether) to obtain 2-(2,6-dichloro-9-(tetrahydro-2H-pyran-2-yl)-9H-purin-8-yl)propan-2-ol as yellow solid (2.9 g, 8.76 mmol, 60%). LCMS [M−84+H]+=247.1.

### Step 3: Synthesis of 2-(2,6-dichloro-9H-purin-8-yl)propan-2-ol hydrochloride

At 0° C., hydrochloric acid (3 mL, 5 mmol, 1 M aqueous solution) was added to a solution of 2-(2,6-dichloro-9-(tetrahydro-2H-pyran-2-yl)-9H-purin-8-yl)propan-2-ol (2.57 g, 7.76 mmol) in a mixture of dichloromethane (10 mL) and methanol (10 mL) and the resulting solution was stirred at 20° C. for 4 h. The resultant mixture was concentrated in vacuo to afford the crude product 2-(2,6-Dichloro-9H-purin-8-yl)-propan-2-ol as yellow solid (2.48 g, 8.75 mmol, 99.8%). LCMS: [M+H]+=247.1.

### Step 4: Synthesis of 2,4-dichloro-6,6-dimethyl-8,9-dihydro-6H-[1,4]oxazino[3,4-e]purine

A mixture of 2-(2,6-dichloro-9H-purin-8-yl)propan-2-ol hydrochloride (2.2 g, 7.76 mmol), 1,2-dibromoethane (5.83 g, 31 mmol) and cesium carbonate (7.59 g, 23.3 mmol) in N,N-dimethylformamide (20 mL) was stirred at 100° C. for 16 h. The mixture was extracted with ethyl acetate (200 mL*2) and washed with water (100 mL*3). The organic layer was dried and concentrated. The resulting residue was purified by column chromatography (SiO2, 0 to 10 to 20% ethyl acetate in petroleum ether) to obtain 2,4-dichloro-6,6-dimethyl-8,9-dihydro-6H-[1,4]oxazino[3,4-e]purine as white solid (686 mg, 29%). LCMS [M+H]+=273.0.

### Step 5: 2-chloro-6,6-dimethyl-4-morpholino-8,9-dihydro-6H-[1,4]oxazino[3,4-e]purine

To a solution of 2,4-dichloro-6,6-dimethyl-8,9-dihydro-6H-[1,4]oxazino[3,4-e]purine (620 mg, 2.27 mmol), morpholine (217 mg, 2.5 mmol) in ethanol (20 mL) was added DIPEA (293 mg, 2.27 mmol). The resulting mixture was stirred at 20° C. for 16 h. After, water (50 mL) was added and the mixture was extracted with dichloromethane (50 mL*3). The organic layer was dried over anhydrous sodium sulfate, filtered and concentrated under reduced pressure. The crude product was purified by flash chromatography (dichloromethane/methanol: 20:1) to give the desired 2-chloro-6,6-dimethyl-4-morpholino-8,9-dihydro-6H-[1,4]oxazino[3,4-e]purine (600 mg, 1.85 mmol, 73.8%) as a white solid. LCMS [M+H]+=324.1.

### Step 6: Synthesis of 6,6-dimethyl-4-morpholino-2-(4-phenyl-1H-pyrazol-1-yl)-8,9-dihydro-6H-[1,4]oxazino[3,4-e]purine

A mixture of 2-chloro-6,6-dimethyl-4-morpholino-8,9-dihydro-6H-[1,4]oxazino[3,4-e]purine (65 mg, 0.2 mmol) and 4-phenyl-1H-pyrazole (31 mg, 0.22 mmol) and cesium carbonate (196 mg, 0.6 mmol) in N,N-dimethylformamide (3 mL) was stirred at 120° C. for 16 h. The resultant crude product was purified by pre-HPLC (BOSTON pHlex ODS 10 μm 21.2×250 mm 120 A. The mobile phase was acetonitrile/0.1% Ammonium bicarbonate) to afford 6,6-dimethyl-4-morpholino-2-(4-phenyl-1H-pyrazol-1-yl)-8,9-dihydro-6H-[1,4]oxazino[3,4-e]purine (93.3 mg, 72%) as white solid.

1H NMR (400 MHz, DMSO-d₆) δ 9.04 (s, 1H), 8.23 (d, J=0.6 Hz, 1H), 7.82-7.75 (m, 2H), 7.41 (t, J=7.7 Hz, 2H), 7.27 (t, J=7.4 Hz, 1H), 4.30 (s, 4H), 4.14 (s, 4H), 3.84-3.74 (m, 4H), 1.59 (s, 6H); LCMS [M+H]+=432.1.

The following compounds were synthesized according to the protocol described for the compound 249:

| Name | Structure | NMR, MS | # |
|---|---|---|---|
| 6,6-dimethyl-4- morpholino-2-(3-phenyl- 1H-pyrazol-1-yl)-8,9- dihydro-6H- [1,4]oxazino[3,4- e]purine | 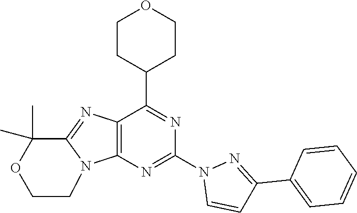 [SMILES: CC1(C)OCCn2c1nc1c(C3CCOCC3)nc(-n3ccc(-c4ccccc4)n3)nc12] | 1H NMR (400 MHz, DMSO-d₆) δ 8.71 (d, J = 2.6 Hz, 1H), 7.95 (d, J = 7.3 Hz, 2H), 7.47 (t, J = 7.5 Hz, 2H), 7.38 (t, J = 7.3 Hz, 1H), 7.02 (d, J = 2.6 Hz, 1H), 4.28 (s, 4H), 4.15 (dd, J = 12.7, 3.9 Hz, 4H), 3.85-3.71 (m, 4H), 1.60 (s, 6H); LCMS [M + H]+ = 432.1. | 250 |

### Preparation of 2-(4-cyclopropyl-1H-pyrazol-1-yl)-6,6-dimethyl-4-morpholino-8,9-dihydro-6H-[1,4]oxazino[3,4-e]purine (Compound 251)

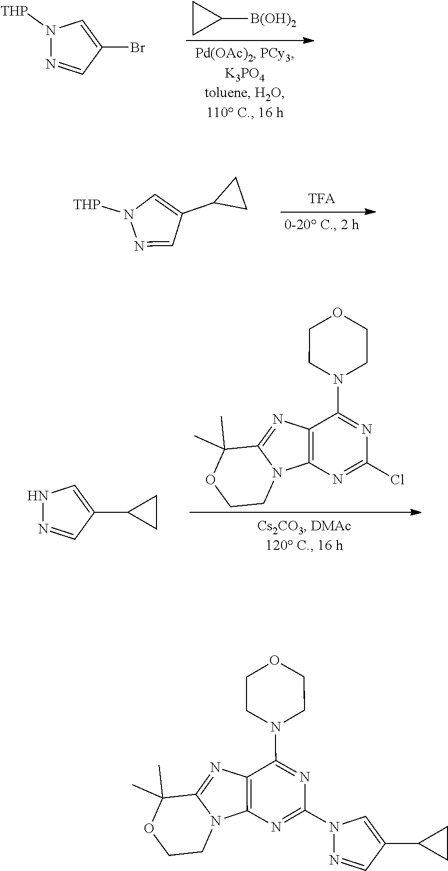

### Step 1: Synthesis of 4-cyclopropyl-1-(tetrahydro-2H-pyran-2-yl)-1H-pyrazole

A solution of 4-bromo-1-(tetrahydro-2H-pyran-2-yl)-1H-pyrazole (231 mg, 1 mmol), cyclopropylboronic acid (258 mg, 3 mmol), palladium acetate (23 mg, 0.1 mmol), tricyclohexyl phosphine (56 mg, 0.2 mmol) and tripotassium phosphate (848 mg, 4 mmol) in water (2 mL) and toluene (20 mL) was stirred at 110° C. for 16 h under argon. The reaction mixture was cooled, concentrated and purified by flash chromatography (Biotage, 80 g silica gel, ethyl acetate/petroleum ether=0%-20%) to give the desired product 4-cyclopropyl-1-(tetrahydro-2H-pyran-2-yl)-1H-pyrazole (240 mg, 89%) as yellow oil. LCMS: (ESI) m/z: 193.2 [M+H]+.

### Step 2: Synthesis of 4-cyclopropyl-1H-pyrazole

To a stirred solution of 4-cyclopropyl-1-(tetrahydro-2H-pyran-2-yl)-1H-pyrazole (60 mg, 0.3 mmol) at 0° C., was added trifluoroacetic acid (2 mL) and the resulting solution was warmed up and stirred at 20° C. for 2 h. The mixture was concentrated in vacuo affording crude product 4-cyclopropyl-1H-pyrazole as yellow oil (105 mg, crude). LCMS: [M+H]+ 109.2.

### Step 3: 2-(4-cyclopropyl-1H-pyrazol-1-yl)-6,6-dimethyl-4-morpholino-8,9-dihydro-6H-[1,4]oxazino[3,4-e]purine

A mixture of 2-chloro-6,6-dimethyl-4-morpholino-8,9-dihydro-6H-[1,4]oxazino[3,4-e]purine (107 mg, 0.33 mmol), 4-cyclopropyl-1H-pyrazole 2,2,2-trifluoroacetate (80 mg, 0.3 mmol) and cesium carbonate (293 mg, 0.9 mmol) in N,N-dimethylformamide (5 mL) was stirred at 120° C. for 16 h. The mixture was purified by pre-HPLC (BOSTON pHlex ODS 10 μm 21.2×250 mm 120 A. The mobile phase was acetonitrile/0.1% Ammonium bicarbonate) to afford product 2-(4-cyclopropyl-1H-pyrazol-1-yl)-6,6-dimethyl-4-morpholino-8,9-dihydro-6H-[1,4]oxazino[3,4-e]purine (56.3 mg, 0.142 mmol, 35.6%) as a white solid. 1H NMR (400 MHz, DMSO-d₆): δ 8.36 (s, 1H), 7.54 (s, 1H), 4.24 (s, 4H), 4.11 (s, 4H), 3.81-3.70 (m, 4H), 1.84-1.75 (m, 1H), 1.58 (s, 6H), 0.91-0.82 (m, 2H), 0.66-0.57 (m, 2H); LCMS: [M+H]+=395.9.

### Synthesis of 1-(9-ethyl-6-morpholino-2-(4-phenyl-1H-pyrazol-1-yl)-9H-purin-8-yl)ethane-1,2-diol (Compound 252) and its enantiomer 1 (Compound 253) and enantiomer 2 (Compound 254)

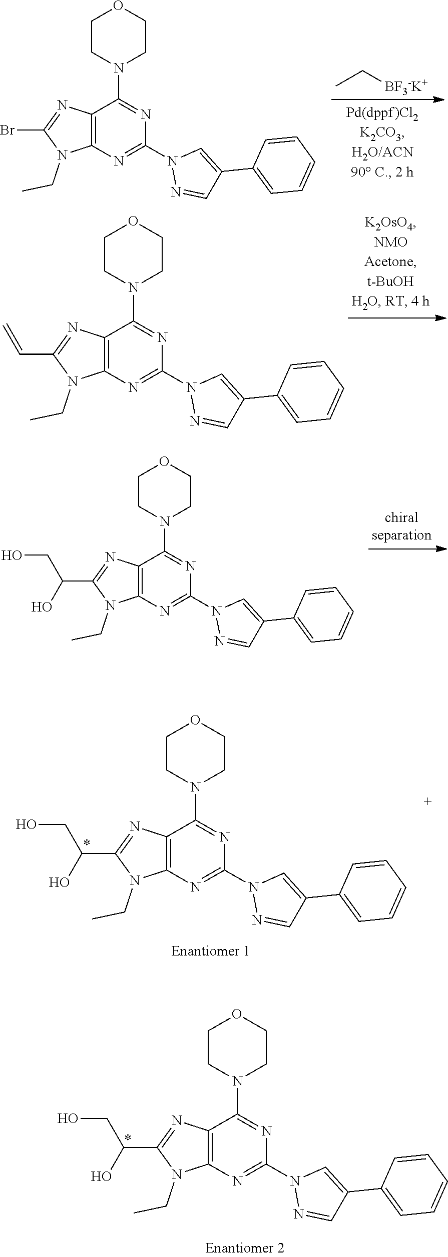

### Step 1: Preparation of 4-(9-ethyl-2-(4-phenyl-1H-pyrazol-1-yl)-8-vinyl-9H-purin-6-yl)morpholine

To a solution of 4-(8-bromo-9-ethyl-2-(4-phenyl-1H-pyrazol-1-yl)-9H-purin-6-yl)morpholine (200.0 mg, 0.44 mmol) in acetonitrile (6 mL) was added potassium vinyltrifluoroborate (69.6 mg, 0.53 mmol), [1,1′-bis(diphenylphosphino)ferrocene]dichloropalladium(II) (32.1 mg, 0.04 mmol) and potassium carbonate (182.1 mg, 1.32 mmol) at 25° C., the reaction was stirred at 90° C. for 2 h under N₂ protection. The mixture was extracted with ethyl acetate (20 mL*2) and washed with water (10 mL*2). The organic layer was dried over Na₂SO₄, and concentrated. The residue was purified by silica gel column chromatography (2% methanol in dichloromethane). The mixture was concentrated to give the product 4-(9-ethyl-2-(4-phenyl-1H-pyrazol-1-yl)-8-vinyl-9H-purin-6-yl)morpholine as white solid (150 mg, 85.0%). LCMS (ESI) m/z: 402.0 [M+H]⁺.

### Step 2: Preparation of 1-(9-ethyl-6-morpholino-2-(4-phenyl-1H-pyrazol-1-yl)-9H-purin-8-yl)ethane-1,2-diol

To a solution of 4-(9-ethyl-2-(4-phenyl-1H-pyrazol-1-yl)-8-vinyl-9H-purin-6-yl)morpholine (800 mg, 2.0 mmol) in acetone (30 mL) was added potassium osmate(VI) dehydrate (147.2 mg, 0.4 mmol), 4-methylmorpholine N-oxide (351.0 mg, 3.0 mmol), 2-methylpropan-2-ol (10 mL) and water (10 mL), the reaction mixture was stirred at 25° C. for 4 h under N₂ protection. After completed, the reaction mixture was filtered, the filtrate was concentrated. The crude product was purified by pre-HPLC (BOSTON pHlex ODS 10 um 21.2×250 mm 120 A. The mobile phase was acetonitrile/0.1% Ammonium bicarbonate to give the desired product 1-(9-ethyl-6-morpholino-2-(4-phenyl-1H-pyrazol-1-yl)-9H-purin-8-yl)ethane-1,2-diol as white solid (85.0 mg, 10%).

Compound 252: ¹H NMR (400 MHz, DMSO-d₆) δ 9.06 (s, 1H), 8.24 (s, 1H), 7.79 (d, J=7.4 Hz, 2H), 7.42 (t, J=7.6 Hz, 2H), 7.27 (t, J=7.3 Hz, 1H), 5.87 (d, J=5.7 Hz, 1H), 4.99-4.74 (m, 2H), 4.35 (d, J=9.0 Hz, 6H), 3.93-3.72 (m, 6H), 1.40 (t, J=7.1 Hz, 3H); LCMS (ESI) m/z: 436.1 [M+H]⁺.

Compound 252 was subjected to chiral prep-HPLC separation [(instrument: SFC-80 (Thar, Waters), Column: AS 20*250 mm, 1 0 um (Daicel), Mobile phase: CO2/methanol (0.2% methanol ammonia)=60/40, Flow rate: 80 g/min, Sample solution: 50 mg dissolved in 15 ml Methanol)] leading to the isolation of two enantiomers as shown below:

Compound 253: ¹HNMR (400 MHz, DMSO) δ 9.06 (s, 1H), 8.24 (s, 1H), 7.79 (d, J=7.2 Hz, 2H), 7.42 (t, J=7.7 Hz, 2H), 7.27 (t, J=7.4 Hz, 1H), 5.87 (d, J=5.7 Hz, 1H), 4.95-4.79 (m, 2H), 4.59-3.94 (m, 6H), 3.91-3.74 (m, 6H), 1.40 (t, J=7.1 Hz, 3H), LCMS (ESI) m/z: 436.1 [M+H]⁺; (Rt: 2.87 min).

Compound 254: ¹HNMR (400 MHz, DMSO) δ 9.06 (s, 1H), 8.23 (d, J=0.7 Hz, 1H), 7.87-7.73 (m, 2H), 7.42 (t, J=7.7 Hz, 2H), 7.27 (t, J=7.4 Hz, 1H), 5.86 (d, J=5.7 Hz, 1H), 4.95-4.73 (m, 2H), 4.35 (d, J=9.2 Hz, 6H), 3.96-3.72 (m, 6H), 1.40 (t, J=7.1 Hz, 3H); LCMS (ESI) m/z: 436.1 [M+H]⁺; (Rt: 3.48 min).

### Synthesis of 2-(9-ethyl-6-morpholino-2-(4-phenyl-1H-pyrazol-1-yl)-9H-purin-8-yl)-2-methoxyethan-1-ol (Compound 255) and 1-(9-ethyl-6-morpholino-2-(4-phenyl-1H-pyrazol-1-yl)-9H-purin-8-yl)-2-methoxyethan-1-ol (Compound 256)

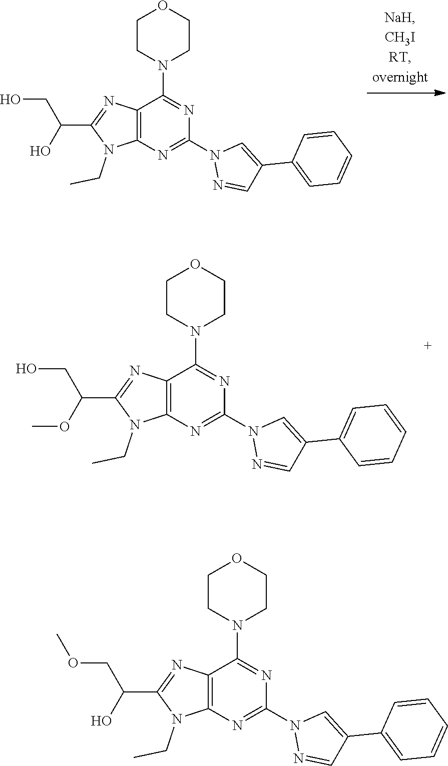 [SMILES: CCn1c(C(O)CO)nc2c(N3CCOCC3)nc(-n3cc(-c4ccccc4)cn3)nc21 ; CCn1c(C(CO)OC)nc2c(N3CCOCC3)nc(-n3cc(-c4ccccc4)cn3)nc21 ; CCn1c(C(O)COC)nc2c(N3CCOCC3)nc(-n3cc(-c4ccccc4)cn3)nc21]

To a solution of 1-(9-ethyl-6-morpholino-2-(4-phenyl-1H-pyrazol-1-yl)-9H-purin-8-yl)ethane-1,2-diol (80.0 mg, 0.18 mmol) in tetrahydrofuran (10 mL) was added iodomethane (26.1 mg, 0.18 mmol and sodium hydride (8.8 mg, 0.22 mmol) at 0° C., the reaction mixture was stirred at 25° C. for 16 h. The reaction was quenched by the addition of the water, extracted with ethyl acetate (20 mL*2) and washed with water (10 mL*2). The organic layer was dried and concentrated. The residue was purified with prep-HPLC (BOSTON pHlex ODS 10 um 21.2×250 mm 120 A. The mobile phase was methyl alcohol and DMF/0.1% Formic acid) to give 2-(9-ethyl-6-morpholino-2-(4-phenyl-1H-pyrazol-1-yl)-9H-purin-8-yl)-2-methoxyethan-1-ol (13.2 mg, 16%) and 1-(9-ethyl-6-morpholino-2-(4-phenyl-1H-pyrazol-1-yl)-9H-purin-8-yl)-2-methoxyethan-1-ol (2.8 mg, 3.4%) as white solids.

Compound 255: ¹H NMR (400 MHz, DMSO-d₆) δ 9.06 (s, 1H), 8.24 (s, 1H), 7.79 (d, J=7.2 Hz, 2H), 7.42 (t, J=7.7 Hz, 2H), 7.27 (t, J=7.4 Hz, 1H), 5.08 (t, J=5.8 Hz, 1H), 4.65 (t, J=6.0 Hz, 1H), 4.40-4.20 (m, 6H), 3.92-3.74 (m, 6H), 3.32 (s, 3H), 1.40 (t, J=7.1 Hz, 3H); LCMS (ESI) m/z: 449.9 [M+H]⁺.

Compound 256: ¹H NMR (400 MHz, DMSO-d₆) δ 9.06 (s, 1H), 8.24 (s, 1H), 7.79 (d, J=7.2 Hz, 2H), 7.42 (t, J=7.7 Hz, 2H), 7.27 (t, J=7.4 Hz, 1H), 5.99 (d, J=6.1 Hz, 1H), 5.00 (dd, J=11.9, 6.2 Hz, 1H), 4.59-3.93 (m, 6H), 3.94-3.67 (m, 7H), 3.34 (s, 3H), 1.39 (t, J=7.1 Hz, 3H); LCMS (ESI) m/z: 449.9 [M+H]⁺.

The following compounds were synthesized according to the protocol described for the Compound 252:

| Name | Structure | NMR, MS | # |
|---|---|---|---|
| 1-(9-ethyl-6- morpholino-2-(4- phenyl-1H- pyrazol-1-yl)-9H- purin-8-yl)ethane- 1,2-diol | 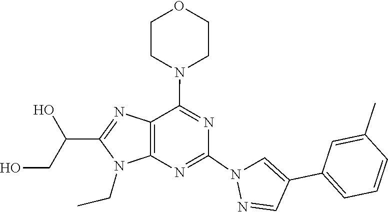 [SMILES: CCn1c(C(O)CO)nc2c(N3CCOCC3)nc(-n3cc(-c4cccc(C)c4)cn3)nc21] | ¹H NMR (400 MHz, DMSO-d₆) δ 9.00 (s, 1H), 8.25 (s, 1H), 8.20 (s, 1H), 7.62-7.51 (m, 2H), 7.44 (d, J = 7.9 Hz, 1H), 5.12 (bs, 1H), 4.77 (dd, J = 7.5, 4.4 Hz, 1H), 4.40-4.25 (m, 6H), 3.77 (s, 4H), 3.46-3.41 (m, 2H), 2.35 (s, 3H), 1.45 (t, J = 7.2 Hz, 3H); LCMS (ESI) m/z: 449.8 [M + H]⁺. | 257 |
| 1-(9-ethyl-6- morpholino-2-(3- (m-tolyl)-1H- pyrazol-1-yl)-9H- purin-8-yl)ethane- 1,2-diol | 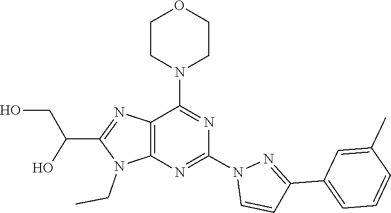 [SMILES: CCn1c(C(O)CO)nc2c(N3CCOCC3)nc(-n3ccc(-c4cccc(C)c4)n3)nc21] | 1H NMR (400 MHz, DMSO-d₆) δ 8.71 (d, J = 2.6 Hz, 1H), 7.81-7.70 (m, 2H), 7.36 (t, J = 7.6 Hz, 1H), 7.19 (d, J = 8.2 Hz, 1H), 7.00 (d, J = 2.6 Hz, 1H), 5.85 (d, J = 5.6 Hz, 1H), 4.92-4.79 (m, 2H), 4.55- 3.95 (m, 6H), 3.81 (dt, J = 9.5, 5.2 Hz, 6H), 2.39 (s, 3H), 1.41 (t, J = 7.1 Hz, 3H); LCMS (ESI) m/z: 450.0 [M + H]⁺. | 258 |

### 1-(9-ethyl-2-(3-(1-methyl-1H-pyrazol-3-yl)phenyl)-6-morpholino-9H-purin-8-yl)ethane-1,2-diol (Compound 259)

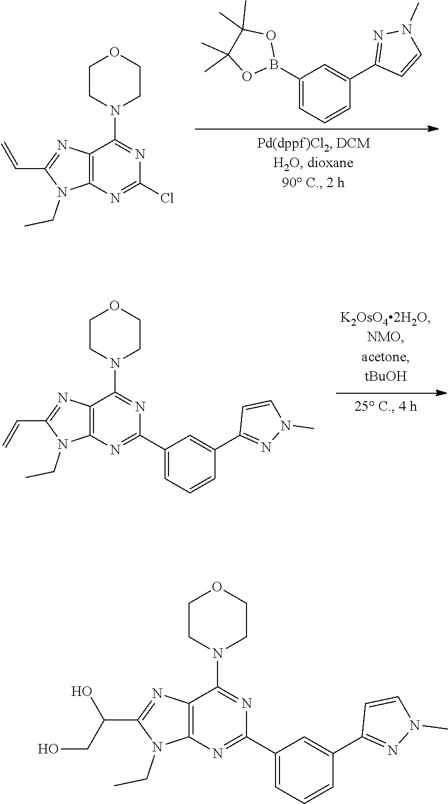 [SMILES: C=Cc1nc2c(N3CCOCC3)nc(Cl)nc2n1CC ; Cn1ccc(-c2cccc(B3OC(C)(C)C(C)(C)O3)c2)n1 ; C=Cc1nc2c(N3CCOCC3)nc(-c3cccc(-c4ccn(C)n4)c3)nc2n1CC ; CCn1c(C(O)CO)nc2c(N3CCOCC3)nc(-c3cccc(-c4ccn(C)n4)c3)nc21]

### Step 1: Synthesis of 4-(9-ethyl-2-(3-(1-methyl-1H-pyrazol-3-yl)phenyl)-8-vinyl-9H-purin-6-yl)morpholine

To a solution of 4-(2-chloro-9-ethyl-8-vinyl-9H-purin-6-yl)morpholine (300 mg, 1.0 mmol) in dioxane (6 mL) and water (2 mL) were added 1-methyl-3-(3-(4,4,5,5-tetramethyl-1,3,2-dioxaborolan-2-yl)phenyl)-1H-pyrazole (436.3 mg, 1.5 mmol), [1,1′-bis(diphenyl phosphino)ferrocene]dichloropalladium(II) (74.9 mg, 0.10 mmol) and potassium carbonate (424.6 mg, 3.06 mmol) at 25° C. and the resultant reaction mixture was stirred at 90° C. for 2 h under nitrogen protection. The mixture was then extracted with ethyl acetate (20 mL*2) and washed with water (10 mL*2). The organic layer was dried over sodium sulfate, filtered and concentrated. The residue was purified by silica gel column chromatography (2% methanol in dichloromethane) to obtain 4-(9-ethyl-2-(3-(1-methyl-1H-pyrazol-3-yl)phenyl)-8-vinyl-9H-purin-6-yl)morpholine as white solid (250 mg, 58.8%). LCMS (ESI) m/z: 416.1 [M+H]⁺.

### Step 2: Synthesis of 1-(9-ethyl-2-(3-(1-methyl-1H-pyrazol-3-yl)phenyl)-6-morpholino-9H-purin-8-yl)ethane-1,2-diol

To a solution of 4-(9-ethyl-2-(3-(1-methyl-1H-pyrazol-3-yl)phenyl)-8-vinyl-9H-purin-6-yl)morpholine (250 mg, 0.60 mmol) in acetone (7 mL) were added potassium osmate(VI) dihydrate (44.4 mg, 0.12 mmol), 4-methylmorpholine n-oxide (124.6 mg, 1.06 mmol), 2-methylpropan-2-ol (1.5 mL) and water (1.5 mL) and the reaction mixture was stirred at 25° C. for 4 h under nitrogen protection. The reaction mixture was then filtered and the filtrate was concentrated. The crude product was purified by prep-HPLC (BOSTON pHlex ODS 10 μm 21.2×250 mm 120 A. The mobile phase was acetonitrile/0.1% Ammonium bicarbonate to obtain 1-(9-ethyl-2-(3-(1-methyl-1H-pyrazol-3-yl)phenyl)-6-morpholino-9H-purin-8-yl)ethane-1,2-diol as white solid (50.9 mg, 18.8%).

¹H NMR (400 MHz, DMSO-d6) δ 8.75 (s, 1H), 8.31 (d, J=7.3 Hz, 1H), 7.99-7.67 (m, 2H), 7.49 (t, J=7.6 Hz, 1H), 6.74 (s, 1H), 5.86 (s, 1H), 4.86 (s, 2H), 4.52-4.19 (m, 6H), 3.99-3.72 (m, 9H), 1.43 (t, J=6.8 Hz, 3H); LCMS (ESI) m/z: 450.4 [M+H]⁺.

### Synthesis of 1-(9-ethyl-2-(3-methoxy-4-phenyl-1H-pyrazol-1-yl)-6-morpholino-9H-purin-8-yl)ethane-1,2-diol (Compound 260)

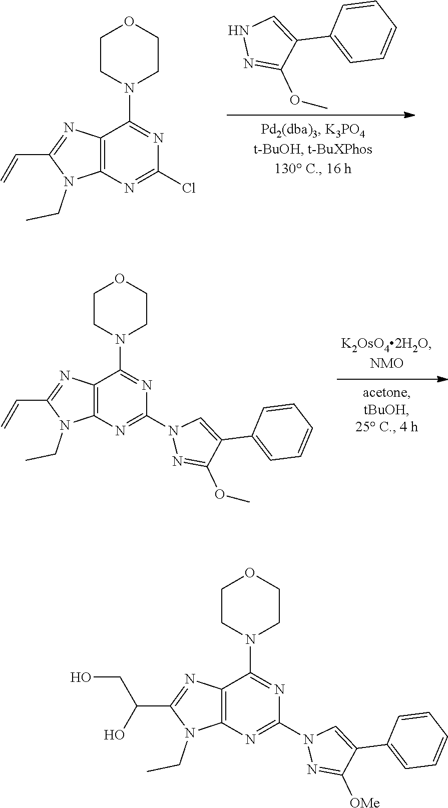 [SMILES: C=Cc1nc2c(N3CCOCC3)nc(Cl)nc2n1CC ; COc1n[nH]cc1-c1ccccc1 ; C=Cc1nc2c(N3CCOCC3)nc(-n3cc(-c4ccccc4)c(OC)n3)nc2n1CC ; CCn1c(C(O)CO)nc2c(N3CCOCC3)nc(-n3cc(-c4ccccc4)c(OC)n3)nc21]

### Step 1: Synthesis of 4-(9-ethyl-2-(3-methoxy-4-phenyl-1H-pyrazol-1-yl)-8-vinyl-9H-purin-6-yl)morpholine

To a solution of 4-(2-chloro-9-ethyl-8-vinyl-9H-purin-6-yl)morpholine (200 mg, 0.68 mmol) in t-butanol (6 mL) were added 3-methoxy-4-phenyl-1H-pyrazole (178.2 mg, 0.75 mmol), tris(dibenzylideneacetone)dipalladium(0) (31.2 mg, 0.04 mmol), tribasic potassium phosphate (217.3 mg, 1.04 mmol) and 2-di-tert-butylphosphino-2′,4′,6′-trisopropylbinphenyl (28.9 mg, 0.05 mmol) at 25° C. and the resultant mixture was stirred at 130° C. for 16 h under nitrogen protection. Then the mixture was extracted with ethyl acetate (20 mL*2), washed with water (10 mL*2), dried over sodium sulfate, and concentrated. The resultant crude product was purified by silica gel column chromatography (3% methanol in dichloromethane) to obtain 4-(9-ethyl-2-(3-methoxy-4-phenyl-1H-pyrazol-1-yl)-8-vinyl-9H-purin-6-yl)morpholine as white solid (100 mg, 34.0%). LCMS (ESI) m/z: 432.4 [M+H]⁺.

### Step 2: Synthesis of 1-(9-ethyl-2-(3-methoxy-4-phenyl-1H-pyrazol-1-yl)-6-morpholino-9H-purin-8-yl)ethane-1,2-diol

To a solution of 4-(9-ethyl-2-(3-methoxy-4-phenyl-1H-pyrazol-1-yl)-8-vinyl-9H-purin-6-yl)morpholine (100 mg, 0.23 mmol) in acetone (5 mL) were added potassium osmate(VI) dihydrate (17.1 mg, 0.05 mmol), 4-methylmorpholine N-oxide (40.7 mg, 0.35 mmol), 2-methylpropan-2-ol (1.5 mL) and water (1.5 mL) and the resultant reaction mixture was stirred at 25° C. for 4 h under nitrogen protection. The mixture was then filtered and the filtrate was concentrated. The resultant crude product was purified by prep-HPLC (BOSTON pHlex ODS 10 μm 21.2×250 mm 120 A. The mobile phase was acetonitrile/0.1% Ammonium bicarbonate) to obtain 1-(9-ethyl-2-(3-methoxy-4-phenyl-1H-pyrazol-1-yl)-6-morpholino-9H-purin-8-yl)ethane-1,2-diol as white solid (44.5 mg, 41.3%).

¹H NMR (400 MHz, DMSO-d₆) δ 8.93 (s, 1H), 7.89-7.73 (m, 2H), 7.40 (t, J=7.7 Hz, 2H), 7.25 (t, J=7.4 Hz, 1H), 5.83 (s, 1H), 4.82 (t, J=6.1 Hz, 2H), 4.45-4.15 (m, 6H), 4.07 (s, 3H), 3.85-3.70 (m, 6H), 1.40 (t, J=7.1 Hz, 3H); LCMS (ESI) m/z: 466.3 [M+H]⁺.

### Preparation of 1-(9-ethyl-6-morpholino-2-(2-phenylpyrimidin-4-yl)-9H-purin-8-yl)ethane-1,2-diol (Compound 261)

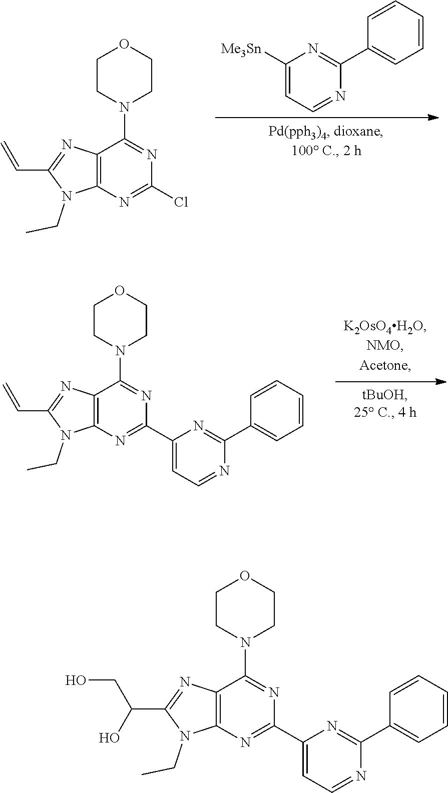

### Step 1: Preparation of 4-(9-ethyl-2-(2-phenylpyrimidin-4-yl)-8-vinyl-9H-purin-6-yl)morpholine

To a solution of 4-(2-chloro-9-ethyl-8-vinyl-9H-purin-6-yl)morpholine (293 mg, 1.0 mmol) in dioxane (15 mL) was added 2-phenyl-4-(trimethylstannyl)pyrimidine (480 mg, 1.5 mmol), tetrakis(triphenylphosphinez)palladium (115 mg, 0.1 mmol) at 25° C. The mixture was stirred at 100° C. for 2 h under N2 protection. The mixture was then extracted with ethyl acetate (20 mL*2) and washed with water (10 mL*2). The organic layer was dried over sodium sulfate, and concentrated. The residue was purified by silica gel column chromatography (5% methanol in dichloromethane) to give the product 4-(9-ethyl-2-(2-phenylpyrimidin-4-yl)-8-vinyl-9H-purin-6-yl)morpholine as white solid (122 mg, 28.3%). LCMS (ESI) m/z: 414.1 [M+H]+

### Step 2: Preparation of 4-(9-ethyl-2-(3-(m-tolyl)-1H-pyrazol-1-yl)-8-vinyl-9H-purin-6-yl)morpholine

To a solution of 4-(9-ethyl-2-(2-phenylpyrimidin-4-yl)-8-vinyl-9H-purin-6-yl)morpholine (122 mg, 0.3 mmol) in acetone (4 mL) was added potassium osmate(VI) dehydrate (22.1 mg, 0.06 mmol), 4-methylmorpholine N-oxide (53 mg, 0.45 mmol), 2-methylpropan-2-ol (1 mL) and Water (1 mL) and the reaction mixture was stirred at 25° C. for 4 h under N2 protection. The reaction mixture was then filtered and the filtrate was concentrated. The crude product was purified by pre-HPLC (BOSTON pHlex ODS 10 μm 21.2×250 mm 120 A. The mobile phase was acetonitrile/0.1% Ammonium bicarbonate to give the desired product 1-(9-ethyl-6-morpholino-2-(2-phenylpyrimidin-4-yl)-9H-purin-8-yl)ethane-1,2-diol as white solid (20.6 mg, 15.4%). 1H NMR (400 MHz, DMSO-d₆) δ 9.04 (d, J=5.1 Hz, 1H), 8.54 (dd, J=7.1, 2.5 Hz, 2H), 8.28 (d, J=5.1 Hz, 1H), 7.58 (dd, J=5.2, 1.7 Hz, 3H), 5.91 (d, J=5.7 Hz, 1H), 4.90 (dt, J=9.7, 5.9 Hz, 2H), 4.45 (dd, J=15.7, 7.0 Hz, 2H), 4.35 (s, 4H), 3.93-3.82 (m, 2H), 3.81-3.77 (m, 4H), 1.46 (t, J=7.1 Hz, 3H); LCMS (ESI) m/z: 448.2 [M+H]+.

### Synthesis of 1-(9-methyl-6-morpholino-2-(1-phenyl-1H-pyrazol-3-yl)-9H-purin-8-yl)ethane-1,2-diol (Compound 262)

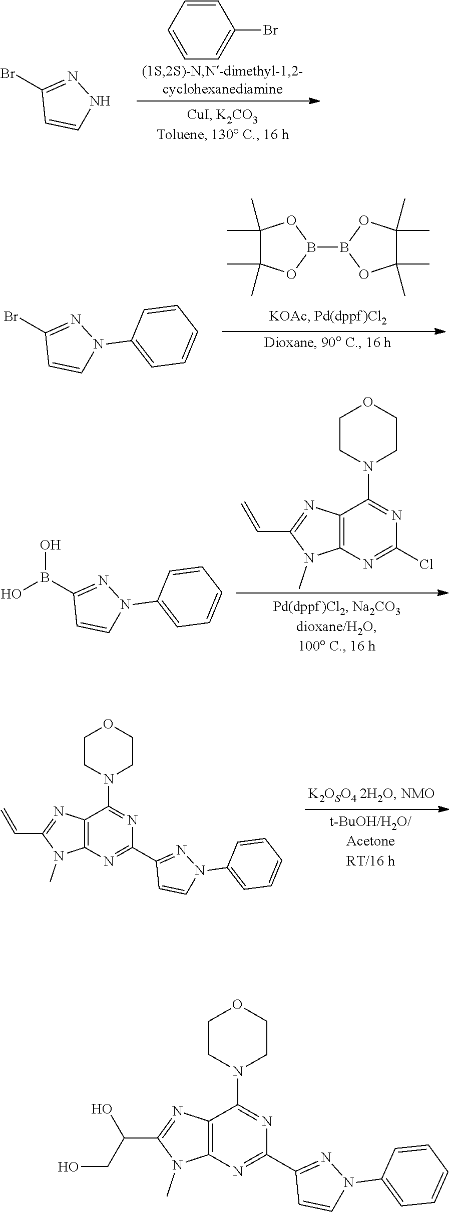

### Step 1: Synthesis of 3-bromo-1-phenyl-1H-pyrazole

A mixture of 3-bromo-1H-pyrazole (500 mg, 3.40 mmol), bromobenzene (1.6 g, 10.21 mmol), (1S,2 S)-N1,N2-dimethylcyclohexane-1,2-diamine (97 mg, 0.68 mmol), KOAc (1.41 g, 10.21 mmol) and CuI (32 mg 0.17 mmol) in toluene (20 mL) was stirred at 130° C. for 16 h under nitrogen protection. The resultant mixture was concentrated and purified by column chromatography (30% EA in PE) to give the target compound as white solid (600 mg, 79%). LCMS (ESI) m/z: 223 [M+H]⁺.

### Step 2: Synthesis of (1-phenyl-1H-pyrazol-3-yl)boronic acid

A mixture of 3-bromo-1-phenyl-1H-pyrazole (150 mg, 0.67 mmol), bis(pinacolato)diboron (512 mg, 2.02 mmol), KOAc (330 mg, 3.36 mmol) and Pd(dpp)Cl₂ (49 mg, 0.07 mmol) in dioxane (8 mL) was stirred at 80° C. for 16 h under nitrogen atmosphere. The resultant mixture was concentrated and purified by column chromatography (5% EA in PE) to obtain the desired compound as white solid (150 mg, 82%). LCMS (ESI) m/z: 189 [M+H]⁺.

### Step 3: Synthesis of 4-(9-methyl-2-(1-phenyl-1H-pyrazol-3-yl)-8-vinyl-9H-purin-6-yl)morpholine

A mixture of 4-(2-chloro-9-methyl-8-vinyl-9H-purin-6-yl)morpholine (100 mg, 0.36 mmol), (1-phenyl-1H-pyrazol-3-yl)boronic acid (101 mg, 0.54 mmol), Cs₂O3 (349 mg, 10.7 mmol) and Pd(dppf)Cl₂ (26 mg, 0.04 mmol) in dioxane (8 mL) and H₂O (1 mL) was stirred at 80° C. for 2 h under nitrogen atmosphere. The mixture was concentrated and purified by column chromatography (30% EA in PE) to obtain the desired product as white solid (80 mg, 58%). LCMS (ESI) m/z: 388 [M+H]⁺.

### Step 4: Synthesis of 1-(9-methyl-6-morpholino-2-(1-phenyl-1H-pyrazol-3-yl)-9H-purin-8-yl)ethane-1,2-diol

To a solution of 4-(9-methyl-2-(1-phenyl-1H-pyrazol-3-yl)-8-vinyl-9H-purin-6-yl)morpholine (40 mg, 0.10 mmol) in acetone (4 mL), water (1 mL) and 2-methylpropan-2-ol (1 ml) were added potassium osmate(VI) dehydrate (7 mg, 0.02 mmol) and 4-methylmorpholine N-oxide (36 mg, 0.51 mmol) and the resultant mixture was stirred at 25° C. for 4 h. It was then was filtered and the filtrate was concentrated. The crude pro duct was purified by pre-HPLC to obtain 1-(9-methyl-6-morpholino-2-(1-phenyl-1H-pyrazol-3-yl)-9H-purin-8-yl)ethane-1,2-diol as white solid (3.1 mg, 8%).

¹H NMR (400 MHz, DMSO-d₆) δ 8.57 (d, J=2.0 Hz, 1H), 7.94 (d, J=7.6 Hz, 2H), 7.57-7.53 (m, 2H), 7.37-7.35 (m, 1H), 7.15 (d, J=2.0 Hz, 1H), 5.84 (d, J=5.2 Hz, 1H), 4.92-4.86 (m, 2H), 4.47-4.25 (m, 4H), 3.86-3.77 (m, 9H); LCMS (ESI) m/z: 422.2 [M+H]⁺.

### Synthesis of 1-(9-ethyl-6-morpholino-2-(4-phenyl-1H-pyrazol-1-yl)-9H-purin-8-yl)ethan-1-ol (Compound 263) and its enantiomer 1 (Compound 264) and enantiomer 2 (Compound 265)

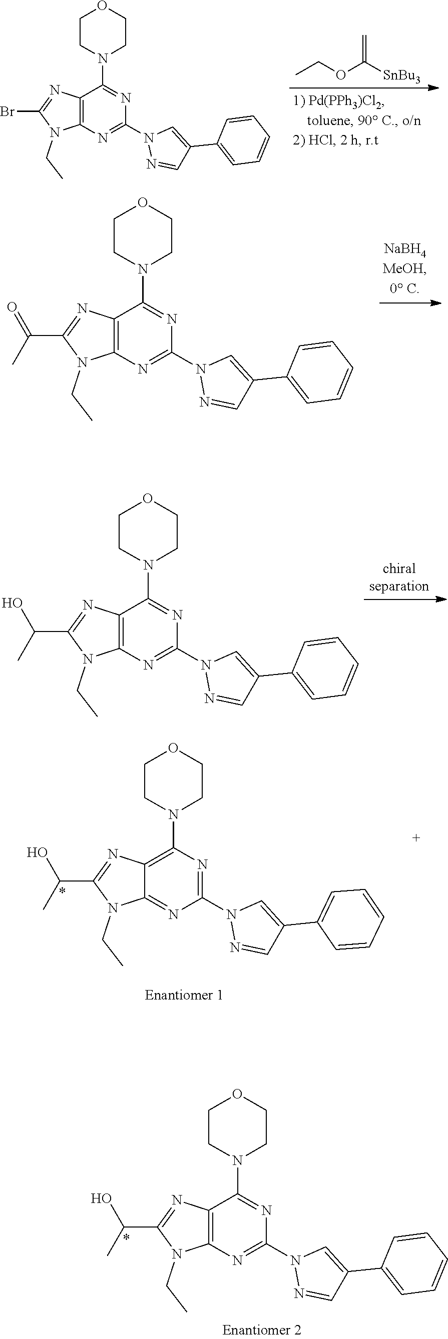

### Step 1: Preparation of 1-(9-ethyl-6-morpholino-2-(4-phenyl-1H-pyrazol-1-yl)-9H-purin-8-yl)ethan-1-one

To a solution of 4-(8-bromo-9-ethyl-2-(4-phenyl-1H-pyrazol-1-yl)-9H-purin-6-yl)morpholine (300.0 mg, 0.66 mmol) in toluene (10 mL) was added tributyl(1-ethoxyvinyl)stannane (263.4 mg, 0.73 mmol) and bis(triphenylphosphine)palladium(II) chloride (48.3 mg, 0.06 mmol) at 25° C., the reaction mixture was stirred at 90° C. for 16 h under N₂ protection. Then hydrochloric acid (10 mL) was added under N₂ protection and the mixture was stirred at 25° C. for 2 h. The mixture was extracted with ethyl acetate (20 mL*2), washed with water (10 mL*2), dried over sodium sulfate and concentrated. The residue was purified by silica gel column chromatography (65% ethyl acetate in petroleum ether) to obtain 1-(9-ethyl-6-morpholino-2-(4-phenyl-1H-pyrazol-1-yl)-9H-purin-8-yl)ethan-1-one as colorless oil (300.0 mg, 95.3%). LCMS (ESI) m/z: 418.0 [M+H]⁺.

### Step 2: Preparation of 1-(9-ethyl-6-morpholino-2-(4-phenyl-1H-pyrazol-1-yl)-9H-purin-8-yl)ethan-1-ol

To a mixture of 1-(9-ethyl-6-morpholino-2-(4-phenyl-1H-pyrazol-1-yl)-9H-purin-8-yl)ethan-1-one (300.0 mg, 0.72 mmol) in methanol (20 mL) was added sodium borohydride (136.0 mg, 1.44 mmol). The mixture was stirred at 0° C. for 0.5 h and at 25° C. for 2 h. Water (20 mL) was added to the mixture and extracted with dichloromethane (20 mL*2) and washed with water (10 mL*2). The organic layer was dried over sodium sulfate and concentrated. The crude product was purified by prep-HPLC (BOSTON pHlex ODS 10 μm 21.2×250 mm 120 A) to obtain 1-(9-ethyl-6-morpholino-2-(4-phenyl-1H-pyrazol-1-yl)-9H-purin-8-yl)ethan-1-ol as white solid (280.0 mg, 92.7%).

Compound 263: ¹H NMR (500 MHz, DMSO) δ 9.05 (s, 1H), 8.23 (s, 1H), 7.79 (d, J=7.4 Hz, 2H), 7.41 (t, J=7.7 Hz, 2H), 7.28 (d, J=7.4 Hz, 1H), 5.73 (bs, 1H), 5.02 (q, J=6.5 Hz, 1H), 4.40-4.20 (m, 6H), 3.77 (t, J=4 Hz, 4H), 1.57 (d, J=6.5 Hz, 3H), 1.41 (t, J=7.1 Hz, 3H). LCMS (ESI) m/z: 420.2 [M+H]⁺.

Compound 263 was subjected to chiral separation (conditions used for the separation of compound 252) to obtain compounds 264 and 265.

Compound 264: ¹HNMR (400 MHz, DMSO) δ 9.06 (s, 1H), 8.23 (s, 1H), 7.79 (d, J=7.2 Hz, 2H), 7.42 (t, J=7.7 Hz, 2H), 7.28 (d, J=7.4 Hz, 1H), 5.72 (d, J=6.3 Hz, 1H), 5.09-4.95 (, 1H), 4.50-4.35 (m, 6H), 3.83-3.72 (m, 4H), 1.57 (d, J=6.5 Hz, 3H), 1.41 (t, J=7.1 Hz, 3H); LCMS (ESI) m/z: 420.2 [M+H]; (Rt: 2.145 min).

Compound 265: HNMR (400 MHz, DMSO) δ 9.06 (d, J=0.7 Hz, 1H), 8.23 (d, J=0.7 Hz, 1H), 7.86-7.73 (m, 2H), 7.42 (t, J=7.7 Hz, 2H), 7.28 (d, J=7.4 Hz, 1H), 5.72 (d, J=6.3 Hz, 1H), 5.02 (t, J=6.4 Hz, 1H), 4.50-4.35 (m, 6H), 3.84-23.72 (m, 4H), 1.57 (d, J=6.5 Hz, 3H), 1.41 (t, J=7.1 Hz, 3H); LCMS (ESI) m/z: 420.2 [M+H](; (Rt: 3.027 min).

The following compounds were chirally resolved according to the protocol described above:

| Name | Structure | NMR, MS | # |
|---|---|---|---|
| 1-(9-ethyl-6- morpholino-2-(3- (m-tolyl)-1H- pyrazol-1-yl)-9H- purin-8-yl)ethane- 1,2-diol (enantiomer 1) | 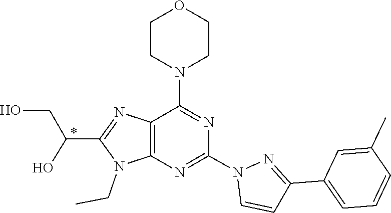 [SMILES: CCn1c(C(O)CO)nc2c(N3CCOCC3)nc(-n3ccc(-c4cccc(C)c4)n3)nc21] | ¹H NMR (400 MHz, DMSO-d₆) δ 8.71 (d, J = 2.6 Hz, 1H), 7.75 (d, J = 13.1 Hz, 2H), 7.36 (t, J = 7.6 Hz, 1H), 7.19 (d, J = 7.5 Hz, 1H), 7.00 (d, J = 2.6 Hz, 1H), 5.85 (d, J = 5.7 Hz, 1H), 4.95-4.76 (m, 2H), 4.36 (d, J = 7.3 Hz, 6H), 3.90-3.73 (m, 6H), 2.39 (s, 3H), 1.41 (t, J = 7.1 Hz, 3H); LCMS (ESI) m/z: 450.0 [M + H]⁺; (Rt: 2.15 min). | 266 |
| 1-(9-ethyl-6- morpholino-2-(3- (m-tolyl)-1H- pyrazol-1-yl)-9H- purin-8-yl)ethane- 1,2-diol (enantiomer 2) | 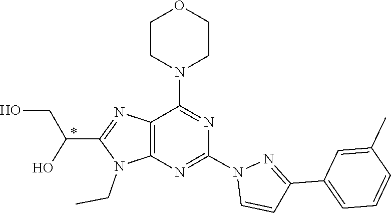 [SMILES: CCn1c(C(O)CO)nc2c(N3CCOCC3)nc(-n3ccc(-c4cccc(C)c4)n3)nc21] | ¹H NMR (400 MHz, DMSO-d₆) δ 8.71 (d, J = 2.6 Hz, 1H), 7.85-7.69 (m, 2H), 7.36 (t, J = 7.6 Hz, 1H), 7.19 (d, J = 7.3 Hz, 1H), 7.00 (d, J = 2.6 Hz, 1H), 5.85 (d, J = 5.7 Hz, 1H), 5.01-4.73 (m, 2H), 4.50-4.05 (m, 6H), 3.91-3.72 (m, 6H), 2.39 (s, 3H), 1.41 (t, J = 7.1 Hz, 3H); LCMS (ESI) m/z: 450.0 [M + H]⁺; (Rt: 2.71 min). | 267 |
| 1-(9-ethyl-6- morpholino-2-(4- (m-tolyl)-1H- pyrazol-1-yl)-9H- purin-8-yl)ethane- 1,2-diol (enantiomer 1) | 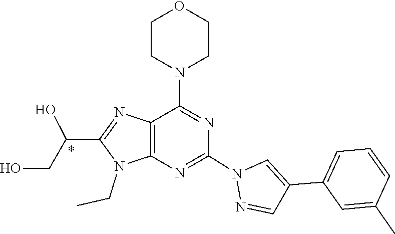 [SMILES: CCn1c(C(O)CO)nc2c(N3CCOCC3)nc(-n3cc(-c4cccc(C)c4)cn3)nc21] | ¹H NMR (400 MHz, DMSO-d₆) δ 8.99 (s, 1H), 8.25 (s, 1H), 8.19(s, 1H), 7.61-7.52 (m, 2H), 7.44 (d, J = 8.1 Hz, 1H), 5.15 (d, J = 4.1 Hz, 1H), 4.75 (t, J = 5.8 Hz, 2H), 4.50-4.15 (m, 6H), 3.86- 3.70 (m, 4H), 3.50-3.35 (m, 2H), 2.36 (s, 3H), 1.45 (t, J = 7.2 Hz, 3H); LCMS (ESI) m/z: 450.2 [M + H]⁺. | 268 |
| 1-(9-ethyl-6- morpholino-2-(4- (m-tolyl)-1H- pyrazol-1-yl)-9H- purin-8-yl)ethane- 1,2-diol (enantiomer 2) | 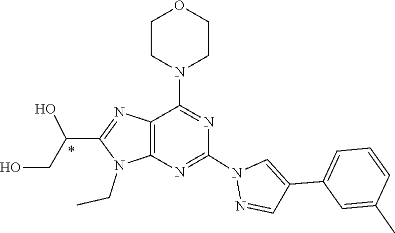 [SMILES: CCn1c(C(O)CO)nc2c(N3CCOCC3)nc(-n3cc(-c4cccc(C)c4)cn3)nc21] | ¹H NMR (400 MHz, DMSO-d₆) δ 9.00 (s, 1H), 8.25 (s, 1H), 8.20(s, 1H), 7.61-7.54 (m, 2H), 7.44 (d, J = 8.1 Hz, 1H), 5.15 (d, J = 4.1 Hz, 1H), 4.75 (t, J = 5.8 Hz, 2H), 4.50-4.15 (m, 6H), 3.78 (s, 4H), 3.50-3.35 (m, 2H), 2.36 (s, 3H), 1.45 (t, J = 7.2 Hz, 3H); LCMS (ESI) m/z: 450.2 [M + H]⁺. | 269 |

### Synthesis of 1-(9-ethyl-2-(3-(1-methyl-1H-pyrazol-3-yl)phenyl)-6-morpholino-9H-purin-8-yl)ethan-1-ol (Compound 270)

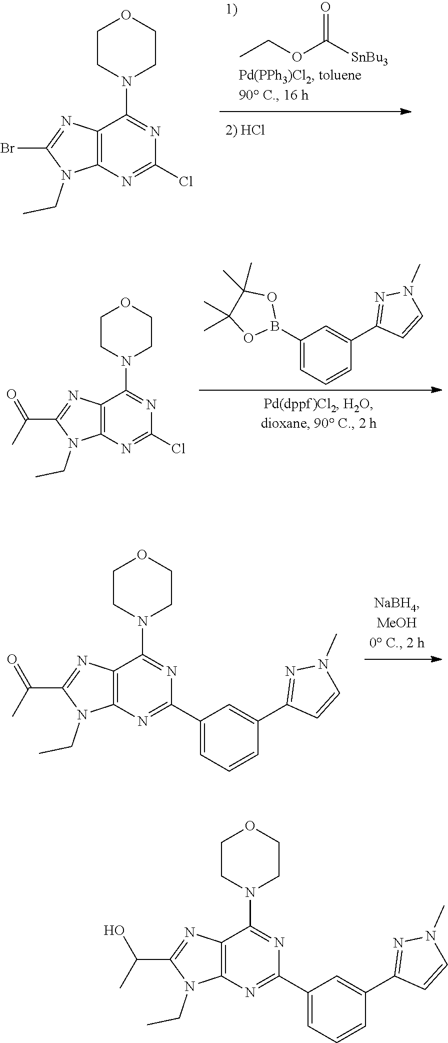

### Step 1: Synthesis of 1-(2-chloro-9-ethyl-6-morpholino-9H-purin-8-yl)ethan-1-one

To a solution of 4-(8-bromo-2-chloro-9-ethyl-9H-purin-6-yl)morpholine (500.0 mg, 1.5 mmol) in toluene (20 mL) was added tributyl(1-ethoxyvinyl)stannane (577.3 mg, 1.6 mmol) and bis(triphenylphosphine)palladium(II) chloride (106.1 mg, 0.15 mmol) at 25° C. and the resultant reaction mixture was stirred at 90° C. for 16 h under nitrogen protection. Then concentrated hydrochloric acid (10 mL) was added the mixture and the mixture was stirred further at 25° C. for 2 h. The mixture was then extracted with ethyl acetate (20 mL*2), the combined organic layer was washed with water (10 mL*2), dried over sodium sulfate and concentrated. The crude product thus obtained was purified by silica gel column chromatography (65% ethyl acetate in petroleum ether) to obtain 1-(2-chloro-9-ethyl-6-morpholino-9H-purin-8-yl)ethan-1-one as white solid. (350.0 mg, 78.2%). LCMS (ESI) m/z: 310.2 [M+H]⁺.

### Step 2: Synthesis of 1-(9-ethyl-2-(3-(1-methyl-1H-pyrazol-3-yl)phenyl)-6-morpholino-9H-purin-8-yl)ethan-1-one

To a solution of 1-(2-chloro-9-ethyl-6-morpholino-9H-purin-8-yl)ethan-1-one (300 mg, 0.9 mol) in dioxane (6 mL) and water (2 mL) were added 1-methyl-3-(3-(4,4,5,5-tetramethyl-1,3,2-dioxaborolan-2-yl)phenyl)-1H-pyrazole (413.7 mg, 1.4 mmol), [1,1′-bis(diphenylphosphino)ferrocene]dichloropalladium(II) (71.0 mg, 0.10 mmol), potassium carbonate (402.6 mg, 2.94 mmol) at 25° C. and the reaction mixture was stirred at 90° C. for 2 h under nitrogen protection. The resultant mixture was extracted with ethyl acetate (20 mL*2) and washed with water (10 mL*2). The organic layer was dried over sodium sulfate, and concentrated. The crude product purified by silica gel column chromatography (4% methanol in dichloromethane) to obtain 1-(9-ethyl-2-(3-(1-methyl-1H-pyrazol-3-yl)phenyl)-6-morpholino-9H-purin-8-yl)ethan-1-one as white solid (300 mg, 71.8%). LCMS (ESI) m/z: 432.4 [M+H]⁺.

### Step 3: Synthesis of 1-(9-ethyl-2-(3-(1-methyl-1H-pyrazol-3-yl)phenyl)-6-morpholino-9H-purin-8-yl)ethan-1-ol

To a solution of 1-(9-ethyl-2-(3-(1-methyl-1H-pyrazol-3-yl)phenyl)-6-morpholino-9H-purin-8-yl)ethan-1-one (300.0 mg, 0.69 mmol) in methanol (20 mL) was added sodium borohydride (121.0 mg, 3.48 mmol) at 0° C. The mixture was stirred at 0° C. for 0.5 h and at 25° C. for 2 h. Then it was extracted with dichloromethane (20 mL*2) and washed with water (10 mL*2). The organic layer was dried over sodium sulfate and concentrated. The crude product was purified by prep-HPLC (BOSTON pHlex ODS 10 um 21.2×250 mm 120 A) to obtain 1-(9-ethyl-2-(3-(1-methyl-1H-pyrazol-3-yl)phenyl)-6-morpholino-9H-purin-8-yl)ethan-1-ol as white solid (98.3 mg, 32.5%).

¹H NMR (400 MHz, DMSO-d₆) δ 8.75 (s, 1H), 8.31 (d, J=7.8 Hz, 1H), 7.85 (d, J=7.7 Hz, 1H), 7.78 (d, J=2.1 Hz, 1H), 7.49 (t, J=7.7 Hz, 1H), 6.74 (d, J=2.2 Hz, 1H), 5.74 (s, 1H), 5.17-4.90 (m, 1H), 4.52-4.10 (m, 6H), 3.92 (s, 3H), 3.83-3.74 (m, 4H), 1.58 (d, J=6.5 Hz, 3H), 1.50-1.39 (m, 3H); LCMS (ESI) m/z: 434.4 [M+H]⁺.

### Synthesis of 1-(1-(9-ethyl-6-morpholino-2-(4-phenyl-1H-pyrazol-1-yl)-9H-purin-8-yl)ethyl)pyridin-4(1H)-one (Compound 271) and its enantiomer 1 (Compound 273) and enantiomer 2 (Compound 274) and synthesis of 4-(9-ethyl-2-(4-phenyl-1H-pyrazol-1-yl)-8-(1-(pyridin-4-yloxy)ethyl)-9H-purin-6-yl)morpholine (Compound 272) and its enantiomer 1 (Compound 275) and enantiomer 2 (Compound 276)

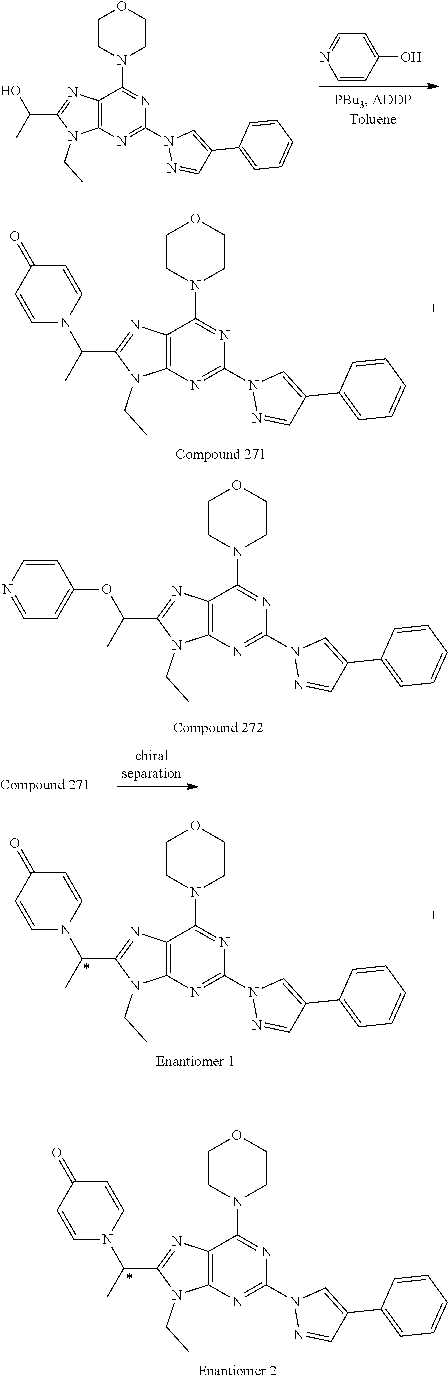

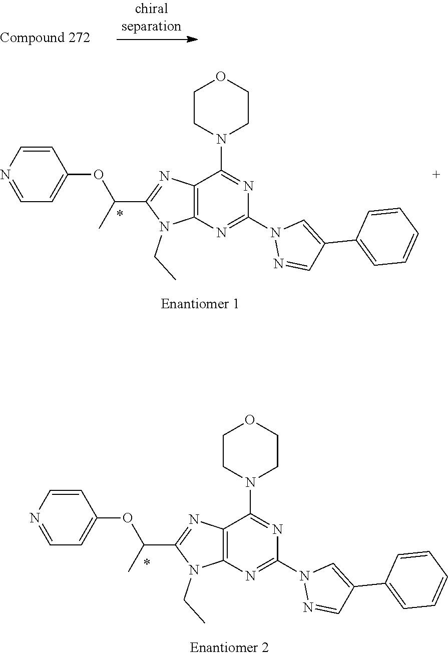 [SMILES: CCn1c(C(C)Oc2ccncc2)nc2c(N3CCOCC3)nc(-n3cc(-c4ccccc4)cn3)nc21 ; CCn1c(C(C)Oc2ccncc2)nc2c(N3CCOCC3)nc(-n3cc(-c4ccccc4)cn3)nc21]

### Step 1: Preparation of 1-(1-(9-ethyl-6-morpholino-2-(4-phenyl-1H-pyrazol-1-yl)-9H-purin-8-yl)ethyl)pyridin-4(1H)-one (Compound 271) and 4-(9-ethyl-2-(4-phenyl-1H-pyrazol-1-yl)-8-(1-(pyridin-4-yloxy)ethyl)-9H-purin-6-yl)morpholine (Compound 272)

To a solution of 1-(9-ethyl-6-morpholino-2-(4-phenyl-1H-pyrazol-1-yl)-9H-purin-8-yl)ethan-1-ol (300 mg, 0.72 mmol), pyridin-4-ol (81.6 mg, 0.86 mmol), 1,1′-(azodicarbonyl)-dipiperidine (363.3 mg, 1.44 mmol) in toluene (20 mL) was added tributylphosphane (218.5 mg, 1.08 mmol) at 25° C. and the resultant mixture was stirred for 2 h. The reaction mixture was then extracted with ethyl acetate (100 ml*2), washed with brine (100 ml), dried, concentrated. The organic layer was dried over sodium sulfate, and concentrated. The crude product was purified by prep-HPLC (BOSTON pHlex ODS 1 0 um 21.2×250 mm 120 A) to give two products; 1-(1-(9-ethyl-6-morpholino-2-(4-phenyl-1H-pyrazol-1-yl)-9H-purin-8-yl)ethyl)pyridin-4(1H)-one (Compound 271) (80.3 mg, 22.6%) and the product 4-(9-ethyl-2-(4-phenyl-1H-pyrazol-1-yl)-8-(1-(pyridin-4-yloxy)ethyl)-9H-purin-6-yl)morpholine (Compound 272) (89.0 mg, 25.1%),

Compound 271: ¹H NMR (500 MHz, DMSO) δ 9.07 (s, 1H), 8.24 (s, 1H), 7.81-7.77 (m, 4H), 7.41 (t, J=7.6 Hz, 2H), 7.27 (t, J=7.4 Hz, 1H), 6.20 (d, J=7.5 Hz, 2H), 5.91 (d, J=6.8 Hz, 1H), 4.66-4.04 (m, 6H), 3.81 (s, 4H), 1.84 (d, J=6.7 Hz, 3H), 1.10 (t, J=7.0 Hz, 3H); LCMS (ESI) m/z: 402.1 [M−95]⁺,

Compound 272: ¹H NMR (500 MHz, DMSO) δ 9.06 (s, 1H), 8.44 (d, J=6.2 Hz, 2H), 8.23 (s, 1H), 7.79 (d, J=7.3 Hz, 2H), 7.41 (t, J=7.7 Hz, 2H), 7.27 (t, J=7.4 Hz, 1H), 7.12 (d, J=6.3 Hz, 2H), 6.12 (q, J=6.2 Hz, 1H), 4.64-4.02 (m, 6H), 3.82-3.75 (m, 4H), 1.78 (d, J=6.4 Hz, 3H), 1.31 (t, J=7.2 Hz, 3H); LCMS (ESI) m/z: 401.9 [M−95]⁺,

Compound 271 was subjected to chiral HPLC separation (conditions used for the compound 252) to afford the following two enantiomers:

Compound 273: ¹H NMR (400 MHz, DMSO) δ 9.07 (s, 1H), 8.24 (s, 1H), 7.90-7.70 (m, 4H), 7.41 (t, J=7.6 Hz, 2H), 7.28 (d, J=7.4 Hz, 1H), 6.14 (d, J=7.6 Hz, 2H), 5.88 (d, J=7.1 Hz, 1H), 4.97-3.99 (m, 6H), 3.81 (s, 4H), 1.83 (d, J=6.6 Hz, 3H), 1.10 (t, J=7.0 Hz, 3H); LCMS (ESI) m/z: 402.1 [M−95]⁺; (Rt: 1.79 min).

Compound 274: ¹H NMR (400 MHz, DMSO) δ 9.07 (s, 1H), 8.24 (s, 1H), 7.93-7.71 (m, 4H), 7.41 (t, J=7.6 Hz, 2H), 7.28 (d, J=7.2 Hz, 1H), 6.14 (d, J=7.7 Hz, 2H), 5.88 (d, J=6.9 Hz, 1H), 4.92-4.03 (m, 6H), 3.81 (s, 4H), 1.83 (d, J=6.6 Hz, 3H), 1.10 (t, J=7.1 Hz, 3H); LCMS (ESI) m/z: 402.2 [M−95]⁺; (Rt: 2.44 min).

Compound 272 was subjected to chiral HPLC separation (conditions used for the compound 252) to afford the following two enantiomers:

Compound 275: ¹H NMR (400 MHz, DMSO) δ 9.07 (s, 1H), 8.44 (d, J=6.0 Hz, 2H), 8.24 (s, 1H), 7.79 (d, J=7.2 Hz, 2H), 7.41 (t, J=7.7 Hz, 2H), 7.27 (t, J=7.4 Hz, 1H), 7.13 (d, J=6.3 Hz, 2H), 6.12 (d, J=6.4 Hz, 1H), 4.45-4.10 (m, 6H), 3.79 (s, 4H), 1.78 (d, J=6.3 Hz, 3H), 1.31 (t, J=7.2 Hz, 3H); LCMS (ESI) m/z: 401.9 [M−95]⁺; (Rt: 2.12 min).

Compound 276: ¹H NMR (400 MHz, DMSO) δ 9.07 (s, 1H), 8.44 (d, J=5.5 Hz, 2H), 8.24 (s, 1H), 7.79 (d, J=7.2 Hz, 2H), 7.41 (t, J=7.7 Hz, 2H), 7.27 (t, J=7.4 Hz, 1H), 7.13 (d, J=6.0 Hz, 2H), 6.13 (t, J=6.3 Hz, 1H), 4.45-4.10 (m, 6H), 3.79 (s, 4H), 1.78 (d, J=6.3 Hz, 3H), 1.31 (t, J=7.1 Hz, 3H); LCMS (ESI) m/z: 401.9 [M−95]⁺; (Rt: 2.93 min).

### Synthesis of 1-(9-ethyl-2-(3-methoxy-4-phenyl-1H-pyrazol-1-yl)-6-morpholino-9H-purin-8-yl)ethan-1-ol (Compound 277)

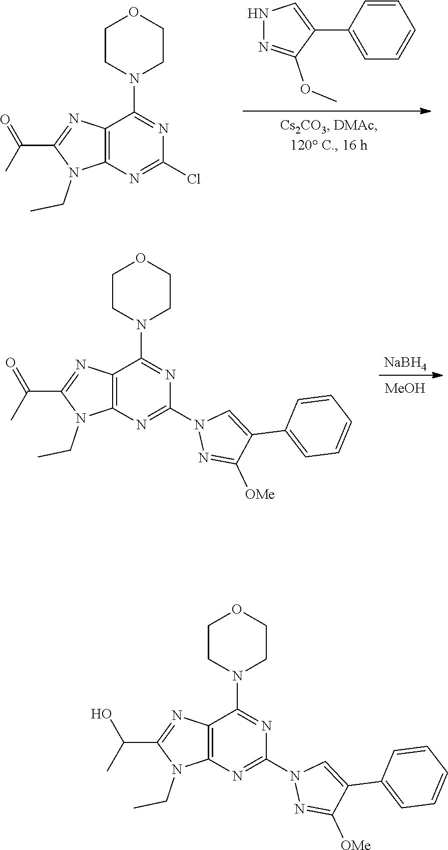

### Step 1: Preparation of 1-(9-ethyl-2-(3-methoxy-4-phenyl-1H-pyrazol-1-yl)-6-morpholino-9H-purin-8-yl)ethan-1-one

To a solution of 1-(2-chloro-9-ethyl-6-morpholino-9H-purin-8-yl)ethan-1-one (185 mg, 0.599 mmol) in N,N-dimethylacetamide (10 mL) were added 3-methoxy-4-phenyl-1H-pyrazole (104 mg, 0.599 mmol) and cesium carbonate (390 mg, 1.198 mmol). The mixture was stirred at 120° C. for 16 h, quenched with water (15 mL) and extracted with ethyl acetate (20 mL*3). The organic layer was combined, washed with brine (30 mL), dried over sodium sulfate, filtered and concentrated. The mixture was concentrated and purified by flash chromatography (Biotage, 80 g silica gel, ethyl acetate/petroleum ether=0%-100%) to give the desired product 1-(9-ethyl-2-(3-methoxy-4-phenyl-1H-pyrazol-1-yl)-6-morpholino-9H-purin-8-yl)ethan-1-one (150 mg, 56%) as yellow oil.

### Step 2: Preparation of 1-(9-ethyl-2-(3-methoxy-4-phenyl-1H-pyrazol-1-yl)-6-morpholino-9H-purin-8-yl)ethan-1-ol

To a solution of 1-(9-ethyl-2-(3-methoxy-4-phenyl-1H-pyrazol-1-yl)-6-morpholino-9H-purin-8-yl)ethan-1-one (150 mg, 0.335 mmol) in methanol (10 mL) was added sodium borohydride (25 mg, 0.670 mmol). The mixture was stirred at 30° C. for 4 h, then quenched with water (15 mL) and extracted with ethyl acetate (20 mL*3). The organic layer was combined, washed with brine (30 mL), dried over sodium sulfate, filtered and concentrated. The residue was purified by Prep-HPLC (0.05% NH4HCO3/H2O:CH3CN=5%-95%) to obtain 1-(9-ethyl-2-(3-methoxy-4-phenyl-1H-pyrazol-1-yl)-6-morpholino-9H-purin-8-yl)ethan-1-ol (86.7 mg, 60%) as a white solid.

¹H NMR (400 MHz, DMSO-d₆) δ 8.92 (s, 1H), 7.81 (d, J=7.2 Hz, 2H), 7.40 (t, J=7.7 Hz, 2H), 7.25 (t, J=7.3 Hz, 1H), 5.70 (d, J=6.3 Hz, 1H), 5.01 (pent, J=6.3 Hz, 1H), 4.35-4.20 (m, 6H), 4.07 (s, 3H), 3.85-3.63 (m, 4H), 1.57 (d, J=6.5 Hz, 3H), 1.41 (t, J=7.1 Hz, 3H). LCMS (ESI) m/z: 450.1 [M+H]+.

### Synthesis of 4-[9-ethyl-8-(4-pyridyl)-2-[3-(3-pyridyl)-1-piperidyl]purin-6-yl]morpholine (Compound 278)

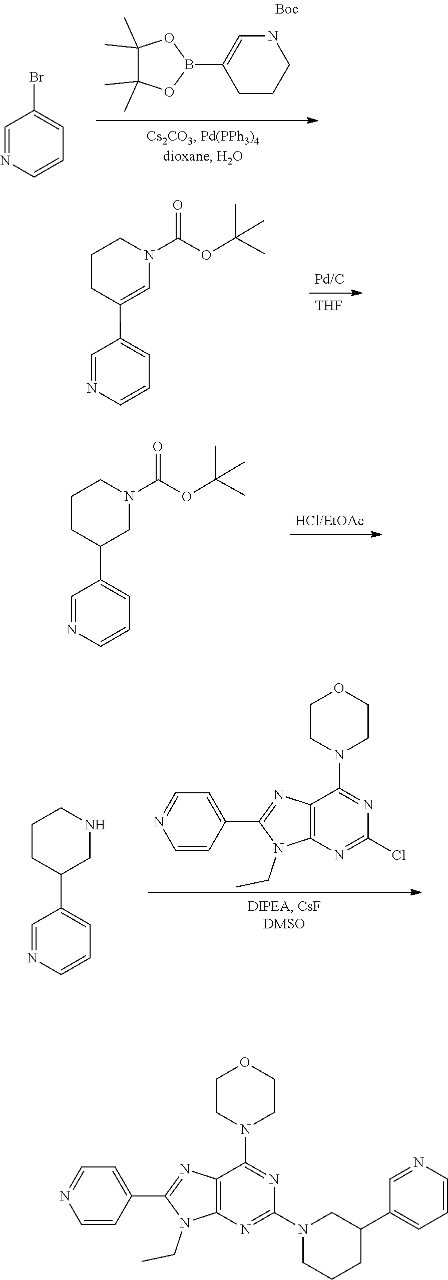

### Step 1: Preparation of tert-butyl 5-(3-pyridyl)-3,4-dihydro-2H-pyridine-1-carboxylate

To a solution of 3-bromopyridine (1.79 g, 11.32 mmol) in 1,4-dioxane (40 mL) and H₂O (20 mL) were added tert-butyl 5-(4,4,5,5-tetramethyl-1,3,2-dioxaborolan-2-yl)-3,4-dihydro-2H-pyridine-1-carboxylate (3.50 g, 11.32 mmol), Cs₂CO₃ (9.22 g, 28.30 mmol, 2.5 eq) and the mixture was thoroughly degassed and purged with nitrogen. Then Pd(PPh₃)₄ (1.31 g, 1.13 mmol) was added to the mixture while maintaining an inert atmosphere. Then the mixture was heated to 90° C. and stirred for 4 h under nitrogen. The mixture was then poured into ice-water (50 mL) and the aqueous phase was extracted with ethyl acetate (50 mL*3). The combined organic phase was washed with brine (20 mL*3), dried with anhydrous Na₂SO₄, filtered and concentrated. The crude product was purified by flash column (ISCO 40 g silica, 0-30% ethyl acetate in petroleum ether, gradient over 20 min) to obtain tert-butyl 5-(3-pyridyl)-3,4-dihydro-2H-pyridine-1-carboxylate (2.9 g, 11.14 mmol, 98%) as a pale yellow oil.

### Step 2: Preparation of tert-butyl 3-(3-pyridyl)piperidine-1-carboxylate

To a solution of tert-butyl 5-(3-pyridyl)-3,4-dihydro-2H-pyridine-1-carboxylate (2.8 g, 10.76 mmol) in THE (25 mL) was added Pd/C (280 mg, 10% purity). Then the mixture was degassed and purged with hydrogen for 3 times, and then it was stirred at 25° C. for 5 h under hydrogen balloon atmosphere. The reaction mixture was filtered with celite and filtrate was concentrated. The product tert-butyl 3-(3-pyridyl)piperidine-1-carboxylate (2.8 g, 10.67 mmol) was obtained as pale yellow oil. LCMS (ESI) m/z: 263.3 [M+H]⁺.

### Step 3: Preparation of 3-(3-piperidyl)pyridine

A mixture of tert-butyl 3-(3-pyridyl)piperidine-1-carboxylate (1.45 g, 5.53 mmol) in HCl/EtOAc (4 M, 15 mL) was stirred at 20° C. for 1 h. The reaction mixture was concentrated to dryness to obtain 3-(3-piperidyl)pyridine (1.14 g, HCl salt) as a brown solid. LCMS (ESI) m/z: 163.2 [M+H]⁺.

### Step 4: Preparation of 4-[9-ethyl-8-(4-pyridyl)-2-[3-(3-pyridyl)-1-piperidyl]purin-6-yl]morpholine

To a solution of 4-[2-chloro-9-ethyl-8-(4-pyridyl)purin-6-yl]morpholine (220 mg, 0.638 mmol) in DMSO (3 mL) were added 3-(3-piperidyl)pyridine (214 mg, 0.638 mmol), cesium fluoride (42 mg, 638 umol) and DIPEA (247 mg, 1.91 mmol). The resultant mixture was stirred at 130° C. for 12 h. Water (3 mL) was added to the reaction mixture and it was extracted with ethyl acetate (3 mL*2). The combined organic layers were washed with brine (3 mL), dried over Na₂SO₄ and concentrated. The residue was purified by prep-HPLC (Waters Xbridge Prep OBD C18 150*40 mm*10 um column; 35-65% acetonitrile in an a 10 mM ammonium bicarbonate solution in water, 8 mingradient) to obtain 4-[9-ethyl-8-(4-pyridyl)-2-[3-(3-pyridyl)-1-piperidyl]purin-6-yl]morpholine (15 mg, 32 umol) as a white solid.

¹H NMR (400 MHz, CHLOROFORM-d) δ 8.74 (d, J=6.0 Hz, 2H), 8.61 (d, J=1.8 Hz, 1H), 8.55-8.49 (m, 1H), 7.74-7.61 (m, 3H), 7.34 (dd, J=4.9, 7.9 Hz, 1H), 4.83 (br t, J=14.5 Hz, 2H), 4.40-4.10 (m, 6H), 3.84 (t, J=4.8 Hz, 4H), 3.07-2.94 (m, 2H), 2.92-2.80 (m, 1H), 2.14-2.06 (m, 1H), 1.90-1.69 (m, 3H), 1.43 (t, J=7.2 Hz, 3H). LCMS (ESI) for (C26H30N8O) [M+H]⁺: 471.3.

### Synthesis of 2-((9-methyl-6-morpholino-8-(pyridin-4-yl)-9H-purin-2-yl)amino)-1-phenylethan-1-ol (Compound 279) and 2-(methyl(9-methyl-6-morpholino-8-(pyridin-4-yl)-9H-purin-2-yl)amino)-1-phenylethan-1-ol (Compound 280)

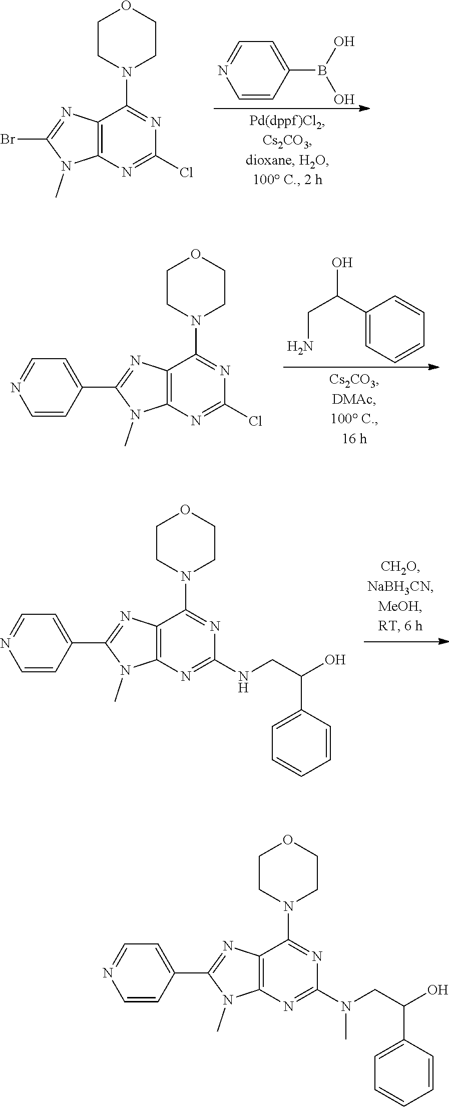 [SMILES: Cn1c(Br)nc2c(N3CCOCC3)nc(Cl)nc21 ; OB(O)c1ccncc1 ; Cn1c(-c2ccncc2)nc2c(N3CCOCC3)nc(Cl)nc21 ; NCC(O)c1ccccc1 ; Cn1c(-c2ccncc2)nc2c(N3CCOCC3)nc(NCC(O)c3ccccc3)nc21 ; CN(CC(O)c1ccccc1)c1nc(N2CCOCC2)c2nc(-c3ccncc3)n(C)c2n1]

### Step 1: Preparation of 4-(2-chloro-9-methyl-8-(pyridin-4-yl)-9H-purin-6-yl)morpholine

To a solution of 4-(8-bromo-2-chloro-9-methyl-9H-purin-6-yl)morpholine (1.0 g, 3.0 mmol) in dioxane/water (10 mL/1 mL) were added pyridin-4-ylboronic acid (0.48 g, 3.9 mmol), cesium carbonate (3.15 g, 10.5 mmol) and bis(diphenylphosphino)ferrocene]dichloropalladium(II) complex (0.22 g, 0.3 mmol) at 25° C. and the reaction was stirred at 100° C. for 2 h under Ar protection. The mixture was filtered and the residue was purified by flash chromatography (petroleum ether:ethyl acetate=2:1) to give 4-(2-chloro-9-methyl-8-(pyridin-4-yl)-9H-purin-6-yl)morpholine as a yellow solid. (350 mg, 35.2%). LCMS (ESI) m/z: 331.1 [M+H]+.

### Step 2: Preparation of 1-((9-methyl-6-morpholino-8-(pyridin-4-yl)-9H-purin-2-yl)amino)-1-phenylethan-1-ol

A mixture of 4-(2-chloro-9-methyl-8-(pyridin-4-yl)-9H-purin-6-yl)morpholine (200 mg, 0.6 mmol), 2-amino-1-phenylethan-1-ol (83 mg, 0.6 mmol) and cesium carbonate (390 mg, 1.2 mmol) in dimethylacetamide (10 mL) was stirred at 140° C. under nitrogen for 3 h. The mixture was filtered and the residue was purified by flash chromatography (petroleum ether:ethyl acetate=2:1) to give product as a yellow solid. (150 mg, 58%).

¹H NMR (400 MHz, DMSO-d₆) δ 8.71 (d, J=6.1 Hz, 2H), 7.82 (dd, J=4.6, 1.6 Hz, 2H), 7.46-7.29 (m, 4H), 7.24 (t, J=7.1 Hz, 1H), 6.47 (dd, J=18.0, 12.7 Hz, 1H), 5.47 (s, 1H), 4.18 (s, 4H), 3.73 (dd, J=18.0, 13.5 Hz 4H), 3.61 (dd, J=12.2, 5.6 Hz, 1H), 3.27 (dd, J=9.3, 6.3 Hz, 1H); LCMS (ESI) m/z: 431.2 [M+H]+.

### Step 3: Preparation of 2-(methyl(9-methyl-6-morpholino-8-(pyridin-4-yl)-9H-purin-2-yl)amino)-1-phenylethan-1-ol

To a solution of 2-((9-methyl-6-morpholino-8-(pyridin-4-yl)-9H-purin-2-yl)amino)-1-phenylethan-1-ol (100 mg, 0.30 mmol) in MeOH (10 mL) was added CH₂O (36.30 mg, 1.21 mmol), and NaBH₃CN (73.81 mg, 1.21 mmol) at 0° C. and the reaction was stirred at room temperature for 6 h under argon atmosphere. The reaction mixture was dilute with ethyl acetate/water (20 mL/20 mL), extracted with ethyl acetate (20 mL*2). The combined organic phase was washed with brine (20 mL), dried over sodium sulfate, filtered and concentrated. The residue was purified by PREP-HPLC and afford 2-(methyl(9-methyl-6-morpholino-8-(pyridin-4-yl)-9H-purin-2-yl)amino)-1-phenylethan-1-ol (10.7 mg, 0.124 mmol, 8.6%) as yellow solid.

¹H NMR (400 MHz, DMSO-d₆) δ 8.71 (d, J=5.4 Hz, 2H), 7.84 (d, J=5.9 Hz, 2H), 7.34 (dd, J=15.0, 7.8 Hz, 4H), 7.24 (t, J=7.0 Hz, 1H), 5.24 (s, 1H), 4.96 (, 1H), 4.20 (, 4H), 3.88 (d, J=14.2 Hz, 1H), 3.82 (, 3H), 3.74 (d, J=4.2 Hz, 4H), 3.54 (d, J=6.9 Hz, 1H), 3.10 (, 3H); LCMS (ESI) m/z: 445.8 [M+H]4.

The following compounds were synthesized according to the protocol described above:

| Name | Structure | NMR, MS | # |
|---|---|---|---|
| 4-(9-methyl-6- morpholino-8- (1H-pyrazol-3-yl)- 9H-purin-2-yl)-2- phenylmorpholine | 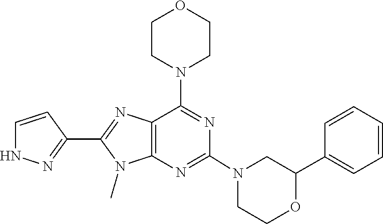 [SMILES: Cn1c(-c2cc[nH]n2)nc2c(N3CCOCC3)nc(N3CCOC(c4ccccc4)C3)nc21] | 1H NMR (400 MHz, DMSO-d₆) δ 13.21 (s, 1H), 7.87 (s, 1H), 7.45 (d, J = 7.0 Hz, 2H), 7.39 (t, J = 7.3 Hz, 2H), 7.33 (t, J = 7.1 Hz, 1H), 6.79 (d, J = 2.3 Hz, 1H), 4.61 (d, J = 13.3 Hz, 1H), 4.57-4.49 (m, 2H), 4.13 (d, J = 29.3 Hz, 4H), 4.06 (s, 1H), 3.90 (s, 3H), 3.73 (d, J = 4.7 Hz, 4H), 3.68 (d, J = 11.5 Hz, 1H), 3.04 (t, J = 10.9 Hz, 1H), 2.83 (dd, J = 12.9, 10.7 Hz, 1H). LCMS (ESI) m/z: 447.2 [M + H]⁺ | 281 |
| 4-(9-methyl-6- morpholino-8- (pyridin-4-yl)-9H- purin-2-yl)-2- phenylmorpholine | 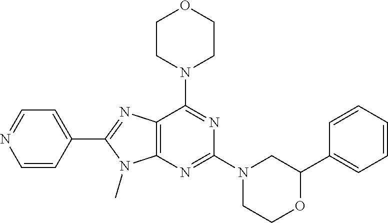 [SMILES: Cn1c(-c2ccncc2)nc2c(N3CCOCC3)nc(N3CCOC(c4ccccc4)C3)nc21] | ¹H NMR (400 MHz, DMSO-d6) δ 8.71 (dd, J = 4.4, 1.6 Hz, 2H), 7.84 (dd, J = 4.4, 1.6 Hz, 2H), 7.32-7.47 (m, 5H), 4.49-4.65 (m, 3H), 4.23 (bs, 4H), 4.06-4.19 (m, 1H), 3.80 (s, 3H), 3.65-3.74 (m, 5H), 3.04-3.10 (m, 1H), 2.83-2.89 (m, 1H); LCMS (ESI) m/z: 458.3 [M + H]+. | 282 |
| 4-(9-ethyl-2-(4- methyl-3- phenylpiperazin- 1-yl)-8-(pyridin-4- yl)-9H-purin-6- yl)morpholine | 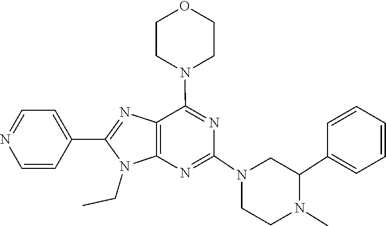 [SMILES: CCn1c(-c2ccncc2)nc2c(N3CCOCC3)nc(N3CCN(C)C(c4ccccc4)C3)nc21] | ¹H NMR (400 MHz, CDCl₃) δ 8.72 (d, J = 6.1 Hz, 2H), 7.65 (dd, J = 4.5, 1.6 Hz, 2H), 7.48-7.29 (m, 5H), 4.77 (d, J = 12.5 Hz, 1H), 4.66 (d, J = 12.5 Hz, 2H), 4.35-4.20 (m, 6H), 3.91-3.74 (m, 4H), 3.20 (d, J = 2.8 Hz, 1H), 3.08-2.88 (m, 3H), 2.44-2.30 (m, 1H), 2.08 (s, 3H), 1.39 (t, J = 7.2 Hz, 3H); LCMS (ESI) m/z: 484.9 [M + H]⁺. | 283 |
| 4-(8-bromo-9- ethyl-6-(3- methylmorpholino)- 9H-purin-2-yl)- 1- methylpiperazin- 2-one | 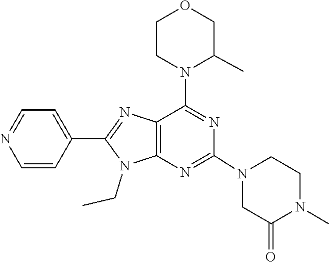 [SMILES: CCn1c(-c2ccncc2)nc2c(N3CCOCC3C)nc(N3CCN(C)C(=O)C3)nc21] | 1H NMR (400 MHz, DMSO-d₆) δ 8.73 (d, J = 5.8 Hz, 2H), 7.77 (d, J = 6.1 Hz, 2H), 5.88-4.51 (m, 1H), 4.28 (dd, J = 16.0, 8.7 Hz, 4H), 4.07-3.89 (m, 3H), 3.74 (t, J = 19.9 Hz, 2H), 3.54-3.39 (m, 5H), 2.89 (s, 3H), 1.29 (dd, J = 6.8, 5.1 Hz, 6H); LCMS (ESI) m/z: 437.3 [M + H]⁺ | 284 |
| 9-methyl-6- morpholino-N-(2- phenylcyclopropyl)- 8-(pyridin-4-yl)- 9H-purin-2-amine | 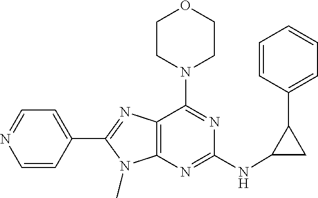 [SMILES: Cn1c(-c2ccncc2)nc2c(N3CCOCC3)nc(NC3CC3c3ccccc3)nc21] | ¹H NMR (400 MHz, DMSO-d₆) δ 8.88 (d, J = 5.1 Hz, 2H), 7.81 (d, J = 5.2 Hz, 2H), 7.27 (t, J = 7.4 Hz, 2H), 7.16 (d, J = 6.9 Hz, 3H), 4.02 (bs, 4H), 3.84 (s, 3H), 3.58 (s, 4H), 2.81 (d, J = 3.4 Hz, 1H), 1.88 (s, 1H), 1.29 (t, J = 4 Hz, 1H), 1.22 (dd, J = 12, 8.0, 1H); LCMS (ESI) m/z: 427.8. [M + H]+. | 285 |
| 4-(2-(3,3- difluoropyrrolidin- 1-yl)-9-methyl-8- (pyridin-4-yl)-9H- purin-6- yl)morpholine | 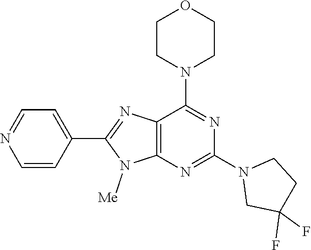 [SMILES: Cn1c(-c2ccncc2)nc2c(N3CCOCC3)nc(N3CCC(F)(F)C3)nc21] | ¹H NMR (400 MHz, CDCl₃) δ 8.73 (d, J = 6.0 Hz, 2H), 7.71 (d, J = 6.0 Hz, 2H), 4.29 (bs, 4H), 3.94- 4.01 (m, 2H), 3.82-3.86 (m, 9H), 2.40-2.50 (m, 2H); LCMS (ESI) m/z: 402.1 [M + H]⁺. | 286 |
| N-cyclopentyl-9- methyl-6- morpholino-8- (pyridin-4-yl)-9H- purin-2-amine | 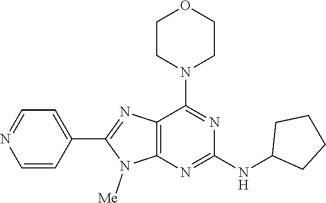 [SMILES: Cn1c(-c2ccncc2)nc2c(N3CCOCC3)nc(NC3CCCC3)nc21] | ¹H NMR (400 MHz, CDCl₃) δ 8.72 (d, J = 5.6 Hz, 2H), 7.69 (d, J = 6.0 Hz, 2H), 4.81-4.79 (m, 1H), 4.32-4.28 (m, 5H), 3.84-3.81 (m, 7H), 2.09-2.05 (m, 2H), 1.75-1.47 (m, 6H); LCMS (ESI) m/z: 380.3 [M + H]⁺. | 287 |

### Synthesis of 4,4′-(9-methyl-8-(1H-pyrazol-3-yl)-9H-purine-2,6-diyl)dimorpholine (Compound 288)

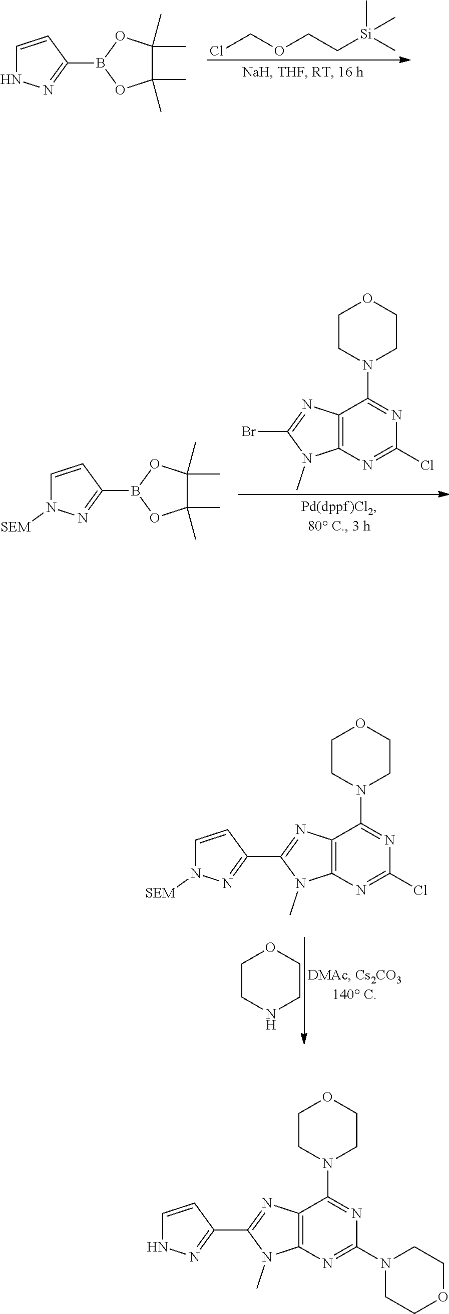

### Step 1: Synthesis of 3-(4,4,5,5-tetramethyl-1,3,2-dioxaborolan-2-yl)-1-((2-(trimethylsilyl)ethoxy)methyl)-1H-pyrazole

To a mixture of 3-(4,4,5,5-tetramethyl-1,3,2-dioxaborolan-2-yl)-1H-pyrazole (2 g, 10.3 mmol) in tetrahydrofuran (60 mL) was slowly added sodium hydride (60% in mineral oil, 1.65 g, 41.2 mmol) at 0° C. The mixture was slowly warmed up to room temperature and stirred for 30 min and further cooled to 0° C. and (2-(chloromethoxy)ethyl)trimethylsilane (3.43 g, 20.6 mmol) was added. The mixture was stirred at room temperature overnight, diluted with water (100 mL) and then extracted with ethyl acetate (100 mL×2). The combined organic layers was washed with brine, dried with sodium sulfate and concentrated to give 3-(4,4,5,5-tetramethyl-1,3,2-dioxaborolan-2-yl)-1-((2-(trimethylsilyl)ethoxy)methyl)-1H-pyrazole as a colorless oil (3.5, g 99%).

### Step 2: Synthesis of 4-(2-chloro-9-methyl-8-(1-((2-(trimethylsilyl)ethoxy)methyl)-1H-pyrazol-3-yl)-9H-purin-6-yl)morpholine

A mixture of 4-(8-bromo-2-chloro-9-methyl-9H-purin-6-yl)morpholine (1 g, 3 mmol), 3-(4,4,5,5-tetramethyl-1,3,2-dioxaborolan-2-yl)-1-((2-(trimethylsilyl)ethoxy)methyl)-1H-pyrazole (3 mmol), [1,1′-bis(diphenylphosphino)ferrocene]dichloropalladium(II) (200 mg, 0.3 mmol) and potassium carbonate (1.1 g, 2.5 mmol) in dioxane (10 mL) and water (0.1 mL) was stirred at 85° C. under nitrogen atmosphere for 16 h. The resultant crude product was purified by flash chromatography on silica gel (Petroleum ether/Ethyl acetate 20:1→10:1→5:1) to give the 4-(2-chloro-9-methyl-8-(1-((2-(trimethylsilyl)ethoxy)methyl)-1H-pyrazol-3-yl)-9H-purin-6-yl)morpholine (200 mg, 15%) as a white solid. LCMS (ESI) m/z: 450.1 [M+H]⁺.

### Step 3: Synthesis of 4,4′-(9-methyl-8-(1H-pyrazol-3-yl)-9H-purine-2,6-diyl)dimorpholine

A mixture of 4-(2-chloro-9-methyl-8-(1-((2-(trimethylsilyl)ethoxy)methyl)-1H-pyrazol-3-yl)-9H-purin-6-yl)morpholine (80 mg, 0.177 mmol), morpholine (31 mg, 0.355 mmol) and cesium carbonate (172 mg, 0.531 mmol) in N,N-dimethylacetamide (5 mL) was stirred at 140° C. for 16 h. The resultant crude product was purified by prep-HPLC to afford 4,4′-(9-methyl-8-(1H-pyrazol-3-yl)-9H-purine-2,6-diyl)dimorpholine (11.6 mg, 18%) as a light yellow solid. ¹H NMR (400 MHz, DMSO-d₆) δ 13.22 (s, 1H), 7.88 (s, 1H), 6.78 (s, 1H), 4.17 (s, 4H), 3.91 (s, 3H), 3.72 (s, 4H), 3.66 (s, 8H); LCMS (ESI) m/z: 371.7 [M+H]⁺.

The following compound was synthesized according to the protocol described above:

| Name | Structure | NMR, MS | # |
|---|---|---|---|
| 4-(7-morpholino-2- (1H-pyrazol-3- yl)thiazolo[5,4- d]pyrimidin-5-yl)-2- phenylmorpholine | 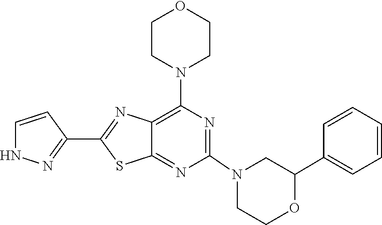 [SMILES: c1ccc(C2CN(c3nc(N4CCOCC4)c4nc(-c5cc[nH]n5)sc4n3)CCO2)cc1] | 1H NMR (400 MHz, CDCl₃) δ 7.64 (d, J = 2.3 Hz, 1H), 7.46 (d, J = 7.4 Hz, 2H), 7.39 (t, J = 7.5 Hz, 2H), 7.33 (t, J = 7.3 Hz, 1H), 6.84 (d, J = 2.2 Hz, 1H), 4.72 (d, J = 13.4 Hz, 1H), 4.64-4.50 (m, 2H), 4.31 (s, 4H),4.20-4.11 (m, 1H), 3.89- 3.83 (m, 4H), 3.83-3.78 (m, 1H), 3.18 (s, 1H), 2.99 (dd, J = 13.2, 10.6 Hz, 1H); LCMS (ESI) m/z: 450.1 [M + H]⁺. | 289 |

### Synthesis of 1-(9-ethyl-6-morpholino-8-(pyridin-4-yl)-9H-purin-2-yl)-4-methyl-3-(pyridin-2-yl)piperazin-2-one (Compound 290)

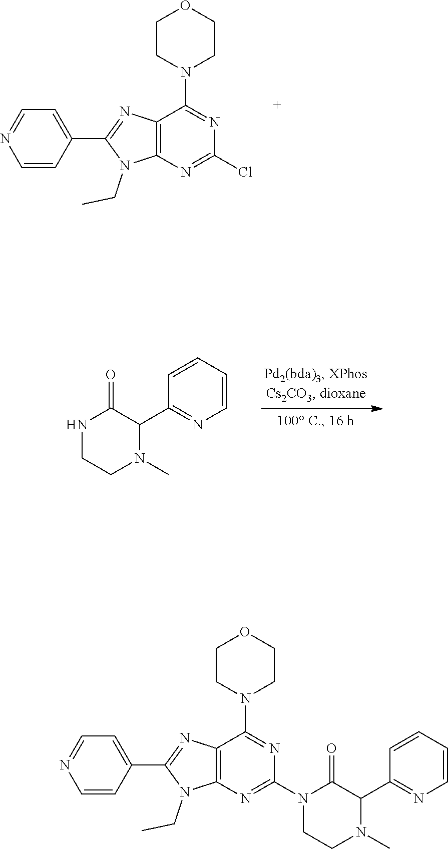 [SMILES: CCn1c(-c2ccncc2)nc2c(N3CCOCC3)nc(Cl)nc21 ; CN1CCNC(=O)C1c1ccccn1 ; CCn1c(-c2ccncc2)nc2c(N3CCOCC3)nc(N3CCN(C)C(c4ccccn4)C3=O)nc21]

A solution of 4-methyl-3-(pyridin-2-yl)piperazin-2-one (80 mg, 0.42 mmol), 4-(2-chloro-9-ethyl-8-(pyridin-4-yl)-9H-purin-6-yl)morpholine (144 mg, 0.42 mmol), tris(dibenzylideneacetone)dipalladium (38 mg, 0.042 mmol), 2-(dicyclohexylphosphino)-2′,4′,6′-tri-i-propyl-1,1′-biphenyl (40 mg, 0.084 mmol) and cesium carbonate (274 mg, 0.84 mmol) in dioxane (10 mL) was stirred at 100° C. for 16 h under Argon. The reaction mixture was concentrated and purified by pre-HPLC (BOSTON pHlex ODS 10 μm 21.2×250 mm 120 A. The mobile phase was acetonitrile/0.1% Ammonium bicarbonate) to give the desired product 1-(9-ethyl-6-morpholino-8-(pyridin-4-yl)-9H-purin-2-yl)-4-methyl-3-(pyridin-2-yl)piperazin-2-one (34.2 mg, 9.3%) as a white solid.

1H NMR (400 MHz, DMSO-d₆) δ 8.78 (dd, J=4.5, 1.6 Hz, 2H), 8.53 (d, J=4.1 Hz, 1H), 7.86-7.76 (m, 3H), 7.44 (d, J=7.8 Hz, 1H), 7.33 (dd, J=6.9, 5.4 Hz, 1H), 4.7-4.10 (m, 6H), 4.09-4.00 (m, 2H), 3.91-3.86 (m, 1H), 3.60-3.35 (m, 4H), 3.21 (d, J=12.0 Hz, 1H), 2.80 (dt, J=16.7, 6.0 Hz, 1H), 2.10 (s, 3H), 1.28 (t, J=7.2 Hz, 3H); LCMS: [M+H]+=499.8.

### Preparation of tert-butyl 3-{3-[9-methyl-6-(morpholin-4-yl)-8-(pyridin-4-yl)-9H-purin-2-yl]-1,2,4-oxadiazol-5-yl}piperidine-1-carboxylate (Compound 291)

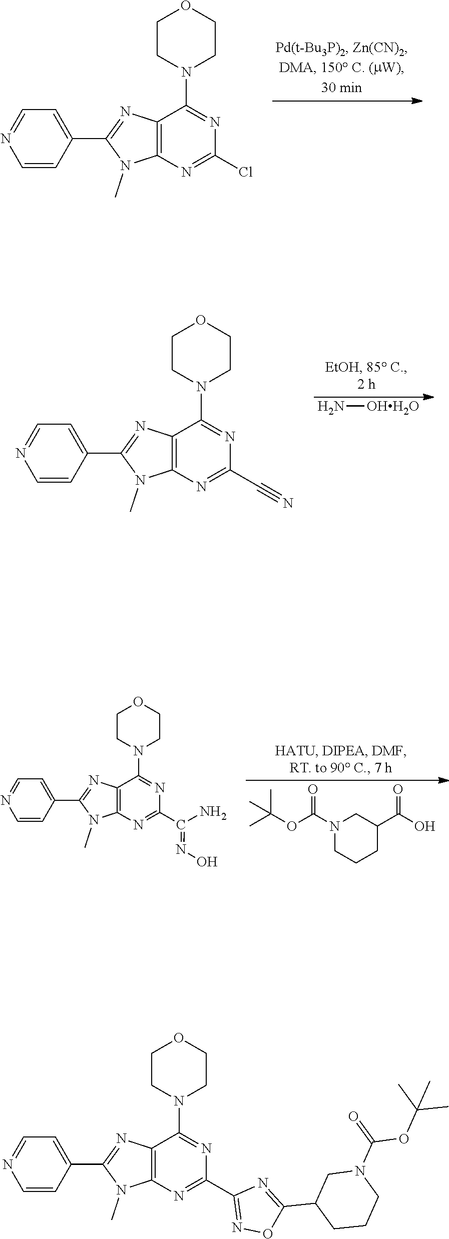

### Step 1: Preparation of 9-methyl-6-morpholino-8-(pyridin-4-yl)-9H-purine-2-carbonitrile

A solution of 4-(2-chloro-9-methyl-8-(pyridin-4-yl)-9H-purin-6-yl)morpholine (500 mg, 1.5 mmol), bis(tri-tert-butylphosphine) palladium(0) (77.0 mg, 0.15 mmol) and zinc cyanide (351 mg, 3.0 mmol) in N,N-dimethylacetamide (6 mL) was irradiated with microwave with stirring at 150° C. for 30 minutes. The reaction mixture was then filtered over celite and washed with ethyl acetate (2×20 mL). The filtrate was concentrated and the crude product obtained was purified by HPLC (SunFire C18, 4.6*50 mm, 3.5 um column Xbridge C18 3.5 μm 4.6×50 mm column. The elution system used was a gradient of 5%-95% over 1.5 min at 2 mL/min and the solvent was acetonitrile/0.01% aqueous ammonium bicarbonate). The product 9-methyl-6-morpholino-8-(pyridin-4-yl)-9H-purine-2-carbonitrile (393 mg, 1.2 mmol, 82%) was obtained as a light yellow solid. LCMS (ESI) m/z: 322.1 [M+H]⁺.

### Step 2: Preparation of (Z)-N′-hydroxy-9-methyl-6-morpholino-8-(pyridin-4-yl)-9H-purine-2-carboximidamide

A solution of 9-methyl-6-morpholino-8-(pyridin-4-yl)-9H-purine-2-carbonitrile (390 mg, 1.2 mmol) and 50% w/v hydroxylamine aqueous solution (241 mg, 3.6 mmol) in ethanol (15 mL) was stirred at 85° C. for 2 h. Water (20 mL) and ethyl acetate (40 mL) were added to the reaction mixture and the layers were separated. The organic layer was separated, dried over sodium sulfate, filtered and concentrated under reduced pressure. The compound (Z)-N′-hydroxy-9-methyl-6-morpholino-8-(pyridin-4-yl)-9H-purine-2-carboximidamide (353 mg, 1.0 mmol, 81%) was obtained as a light yellow solid and carried onto the next step without further purification. LCMS (ESI) m/z: 355.1 [M+H]⁺.

### Step 3: Preparation of tert-butyl 3-{3-[9-methyl-6-(morpholin-4-yl)-8-(pyridin-4-yl)-9H-purin-2-yl]-1,2,4-oxadiazol-5-yl}piperidine-1-carboxylate

To a solution of (Z)-N′-hydroxy-9-methyl-6-morpholino-8-(pyridin-4-yl)-9H-purine-2-carboximidamide (180 mg, 1.0 mmol), 1-(tert-butoxycarbonyl)piperidine-3-carboxylic acid (228 mg, 1.0 mmol), N,N-diisopropylethylamine (387 mg, 3.0 mmol) in N,N-dimethylformamide (5 mL) was added 1-[Bis(dimethylamino)methylene]-1H-1,2,3-triazolo[4,5-b]pyridinium 3-oxid hexafluorophosphate (569 mg, 1.5 mmol). The reaction mixture was stirred at room temperature for 1 h, then heated to 90° C. and stirred for an additional 6 h. Water (20 mL) and ethyl acetate (100 mL) were then added to the reaction mixture, the organic layer was separated, washed with brine (100 mL), dried over sodium sulfate, filtered, and concentrated under reduced pressure. The resultant product was purified by HPLC (SunFire C18, 4.6*50 mm, 3.5 um column Xbridge C18 3.5 μm 4.6×50 mm column. The elution system used was a gradient of 5%-95% over 1.5 min at 2 mL/min and the solvent was acetonitrile/0.01% aqueous ammonium bicarbonate) to afford tert-butyl 3-{3-[9-methyl-6-(morpholin-4-yl)-8-(pyridin-4-yl)-9H-purin-2-yl]-1,2,4-oxadiazol-5-yl}piperidine-1-carboxylate (200 mg, 0.37 mmol, 37%) as a white solid. ¹H NMR (400 MHz, Dimethylsulfoxide-d₆) δ 8.80 (d, J=6.0 Hz, 2H), 7.93 (dd, J=4.5, 1.6 Hz, 2H), 4.23 (m, 5H), 3.96 (s, 3H), 3.84-3.74 (m, 4H), 3.60 (s, 2H), 3.16 (s, 2H), 2.16 (s, 1H), 1.92 (s, 1H), 1.75 (s, 1H), 1.52 (dd, J=9.7, 3.6 Hz, 1H), 1.39 (s, 9H); LCMS (ESI) m/z: 355.1 [M+H]⁺.

### Synthesis of 4-(2-(5-phenyl-1,2,4-oxadiazol-3-yl)-8-(pyridin-4-yl)-9H-purin-6-yl)morpholine (Compound 292)

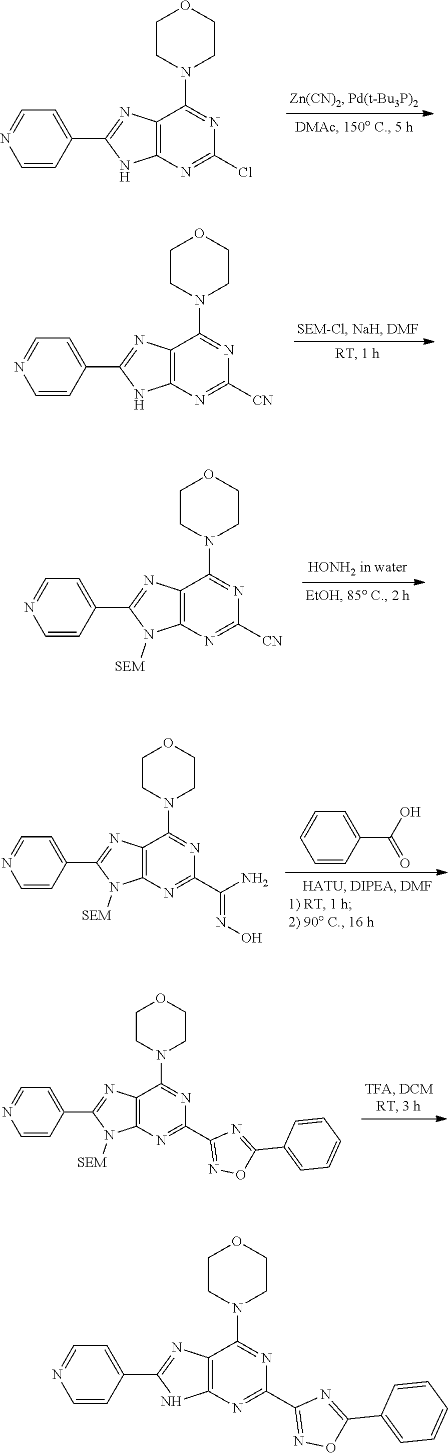

### Step 1: Synthesis of 6-morpholino-8-(pyridin-4-yl)-9H-purine-2-carbonitrile

A mixture of 4-(2-chloro-8-(pyridin-4-yl)-9H-purin-6-yl)morpholine (150 mg, 0.45 mmol), zinc cyanide (63 mg, 0.54 mmol) and bis(tri-tert-butylphosphine)palladium (24 mg, 0.045 mmol) in dry dimethylacetamide (6 mL) under nitrogen protection was stirred at 150° C. for 4 h. The mixture was cooled to room temperature, quenched with water (10 mL) and filtered, and the residue was washed with water and dried to give the crude product as brown solid (150 mg, crude). LCMS (ESI) m/z: 308 [M+H]⁺.

### Step 2: Synthesis of 6-Morpholino-8-(pyridin-4-yl)-9-((2-(trimethylsilyl)ethoxy)methyl)-9H-purine-2-carbonitrile

To a solution of 6-morpholino-8-(pyridin-4-yl)-9H-purine-2-carbonitrile (150 mg, 0.49 mmol) in tetrahydrofuran (10 mL) was added sodium hydride (60% in oil, 29 mg, 0.74 mmol), after stirring at room temperature for 10 minutes, 2-(trimethylsilyl)ethoxymethyl chloride (122 mg, 0.73 mmol) was added. The resulting mixture was stirred at room temperature for 2 h, then quenched with methanol (5 mL) and concentrated under reduced pressure. The crude product was then purified by column chromatography (20% ethyl acetate in petroleum ether) to obtain the product as white solid (150 mg, 70.2%). LCMS (ESI) m/z: 438 [M+H]⁺.

### Step 3: Synthesis of (Z)-N′-hydroxy-6-morpholino-8-(pyridin-4-yl)-9-((2-(trimethylsilyl)ethoxy)methyl)-9H-purine-2-carboximidamide

A mixture of 6-morpholino-8-(pyridin-4-yl)-9-((2-(trimethylsilyl)ethoxy)methyl)-9H-purine-2-carbonitrile (150 mg, 0.34 mmol) and hydroxylamine (50% in water, 0.034 mL, 34 mg) in ethanol (5 mL) was stirred at 80° C. for 2 h. The mixture was concentrated under reduced pressure to give the crude product as yellow oil (150 mg, crude), which used for next step directly without further purification. LCMS (ESI) m/z: 471 [M+H]⁺.

### Step 4: Synthesis of 4-(2-(5-Phenyl-1,2,4-oxadiazol-3-yl)-8-(pyridin-4-yl)-9-((2-(trimethylsilyl)ethoxy)methyl)-9H-purin-6-yl)morpholine

A mixture of (2)-N′-hydroxy-6-morpholino-8-(pyridin-4-yl)-9-((2-(trimethylsilyl)ethoxy)methyl)-9H-purine-2-carboximidamide (150 mg, 0.32 mmol), benzoic acid (39 mg, 0.32 mmol), 1-[bis(dimethylamino)methylene]-1H-1,2,3-triazolo[4,5-b]pyridinium 3-oxid hexafluorophosphate (182 mg, 0.48 mmol) and N,N-diisopropylethylamine (83 mg, 0.64 mmol) in N,N-dimethylformamide (10 mL) was stirred at room temperature for 1 h at 90° C. for 16 h. The mixture was cooled to room temperature, quenched with water (10 mL) and extracted with ethyl acetate (10 mL*3). The combined organic phases were washed with water and brine, dried over sodium sulfate, filtered and concentrated under reduced pressure to give the crude product as yellow oil (100 mg, crude). LCMS (ESI) m/z: 557 [M+H]⁺.

### Step 5: Synthesis of 4-(2-(5-Phenyl-1,2,4-oxadiazol-3-yl)-8-(pyridin-4-yl)-9H-purin-6-yl)morpholine

A mixture of 4-(2-(5-phenyl-1,2,4-oxadiazol-3-yl)-8-(pyridin-4-yl)-9-((2-(trimethylsilyl)ethoxy)methyl)-9H-purin-6-yl)morpholine (100 mg, 0.18 mmol) and trifluoroacetic acid (2 mL) in dichloromethane (5 mL) was stirred at room temperature for 3 h. The mixture was concentrated and the residue was quenched by saturated sodium bicarbonate solution till pH>7 and extracted by ethyl acetate (10 mL*3). The combined organic phases were concentrated and the crude product was purified by prep-HPLC (Boston C18 21*250 mm 10 μm column. The mobile phase was acetonitrile/0.01% aqueous ammonium bicarbonate) to obtain the desired product as white solid (9.2 mg, 12.0%).

¹H NMR (400 MHz, DMSO-d₆) δ 14.39 (s, 1H), 8.78 (d, J=6.0 Hz, 2H), 8.21 (d, J=6.8 Hz, 2H), 8.07 (d, J=6.0 Hz, 2H), 7.79-7.73 (m, 1H), 7.71-7.65 (m, 2H), 4.38 (s, 4H), 3.82 (t, J=4.8 Hz, 4H); LCMS (ESI) m/z: 427.8 [M+H]⁺.

### Synthesis of 4-(9-ethyl-2-(5-phenyl-1,3,4-oxadiazol-2-yl)-8-(pyridin-4-yl)-9H-purin-6-yl)morpholine (Compound 293)

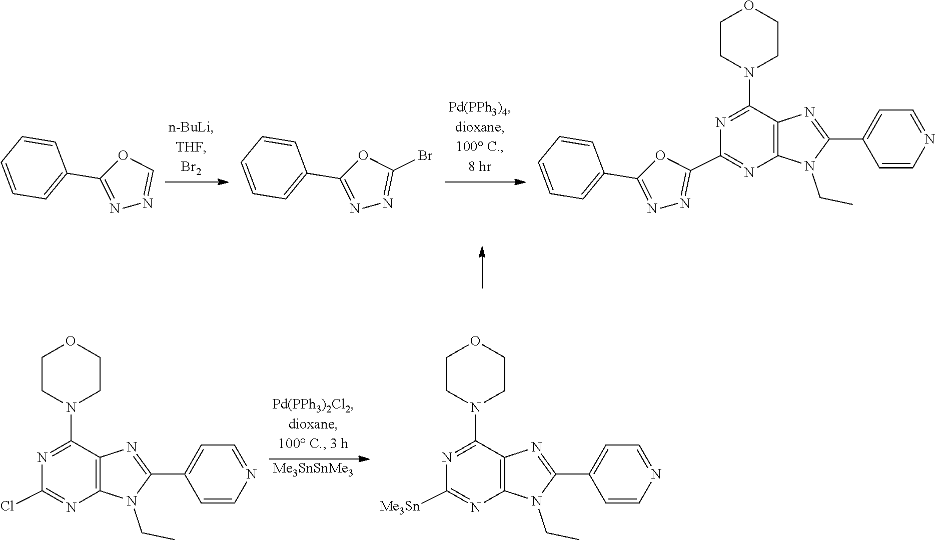

### Step 1: Preparation of 2-bromo-5-phenyl-1,3,4-oxadiazole

To a solution of 2-phenyl-1,3,4-oxadiazole (150 mg, 1.03 mmol) in tetrahydrofuran (10 mL) was added n-butyllithium (1.0 ml, 2.06 mmol) at −78° C. The mixture was stirred at −45° C. for 0.5 h. Then bromine (330 mg, 2.06 mmol was added at −78° C. and the resultant mixture was warmed up and stirred at room temperature for 8 h. It was quenched with water (15 mL) and extracted with ethyl acetate (20 mL*3). The organic layer was combined, washed with brine (30 mL), dried over sodium sulfate, filtered and concentrated. The residue was purified by flash chromatography on silica gel (petroleum ether:ethyl acetate=75:25) to give product as yellow oil (110 mg, 47.5%); LCMS (ESI) m/z: 225.9[M+]+.

### Step 1a: Preparation of 4-(9-ethyl-8-(pyridin-4-yl)-2-(trimethylstannyl)-9H-purin-6-yl)morpholine

To a solution of 4-(2-chloro-9-ethyl-8-(pyridin-4-yl)-9H-purin-6-yl)morpholine (300 mg, 0.872 mmol) in dioxane (10 mL) was added hexamethyldistannane (285 mg, 0.872 mmol) and bis(triphenylphosphine)palladium(II) chloride (70 mg, 0.10 mmol). The mixture was stirred at 100° C. for 2 h, then cooled and concentrated. The residue was purified by flash chromatography on silica gel (petroleum ether:ethyl acetate=5:95) to obtain the product as yellow solid. (150 mg, 47.5%); LCMS (ESI) m/z: 473.2/475.0 [M]+.

### Step 2: Preparation of 4-(9-ethyl-2-(5-phenyl-1,3,4-oxadiazol-2-yl)-8-(pyridin-4-yl)-9H-purin-6-yl)morpholine

To a solution of 4-(9-ethyl-8-(pyridin-4-yl)-2-(trimethylstannyl)-9H-purin-6-yl)morpholine (150 mg) in dioxane (10 mL) were added 2-bromo-5-phenyl-1,3,4-oxadiazole (110 mg, 0.493 mmol) and tetrakis(triphenylphosphine)palladium (58 mg, 0.05 mmol) and the resultant mixture was stirred at 100° C. for 16 h. The reaction mixture was concentrated, the crude residue was purified by Prep-HPLC (BOSTON pHlex ODS 10 μm 21.2×250 mm 120 A. The mobile phase was acetonitrile/0.1% Ammonium bicarbonate) to afford 4-(9-(difluoromethyl)-2-(2-phenylpyrimidin-4-yl)-8-(pyridin-4-yl)-9H-purin-6-yl)morpholine (17.7 mg, 0.048 mmol) as white solid. ¹H NMR (400 MHz, DMSO-d₆) δ 8.83 (dd, J=4.5, 1.6 Hz, 2H), 8.14 (dd, J=7.7, 1.8 Hz, 2H), 7.88 (dd, J=4.5, 1.6 Hz, 2H), 7.68 (t, J=6.3 Hz, 3H), 4.53-4.47 (m, 2H), 4.46 (s, 4H), 3.91-3.76 (m, 4H), 1.37 (t, J=7.2 Hz, 3H); LCMS (ESI) m/z: 454.8[M+H]+.

The following compounds were synthesized according to the protocol described for the Compound 291:

| Name | Structure | 1H NMR Data | # |
|---|---|---|---|
| 4-(9-methyl-2- (5-(1- methylpiperidin- 3-yl)-1,2,4- oxadiazol-3-yl)- 8-(pyridin-4-yl)- 9H-purin-6- yl)morpholine | 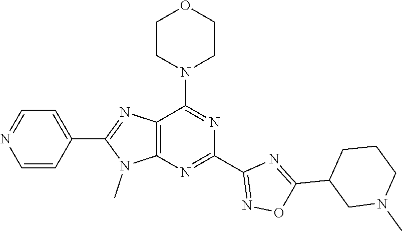 [SMILES: CN1CCCC(c2nc(-c3nc(N4CCOCC4)c4nc(-c5ccncc5)n(C)c4n3)no2)C1] | ¹H NMR (400 MHz, Dimethylsulfoxide-d₆) δ 8.81 (dd, J = 4.5, 1.6 Hz, 2H), 7.93 (dd, J = 4.5, 1.6 Hz, 2H), 4.33 (s, 4H), 3.96 (s, 3H), 3.88-3.68 (m, 4H), 3.02 (d, J = 9.5 Hz, 1H), 2.62 (s, 1H), 2.37 (s, 1H), 2.23 (s, 3H), 2.06 (d, J = 8.3 Hz, 2H), 1.82-1.71 (m, 1H), 1.64 (d, J = 8.1 Hz, 2H); LCMS (ESI) m/z: 462.1 [M + H]⁺. | 294 |
| 4-(9-methyl-2- (5-(piperidin-3- yl)-1,2,4- oxadiazol-3-yl)- 8-(pyridin-4-yl)- 9H-purin-6- yl)morpholine | 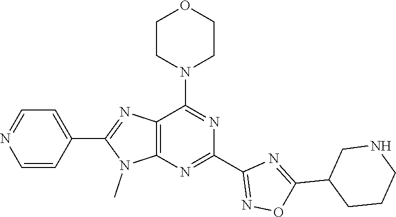 [SMILES: Cn1c(-c2ccncc2)nc2c(N3CCOCC3)nc(-c3noc(C4CCCNC4)n3)nc21] | ¹H NMR (400 MHz, Dimethylsulfoxide-d₆) δ 8.80 (dd, J = 4.5, 1.6 Hz, 2H), 7.93 (dd, J = 4.5, 1.6 Hz, 2H), 4.33 (s, 4H), 3.96 (s, 3H), 3.88-3.63 (m, 4H), 3.19 (m, 2H), 2.94-2.76 (m, 2H), 2.61-2.53 (m, 1H), 2.15 (d, J = 9.1 Hz, 1H), 1.81 (td, J = 14.4, 3.9 Hz, 1H), 1.73- 1.63 (m, 1H), 1.57-1.38 (m, 1H); LCMS (ESI) m/z: 448.2 [M + H]⁺. | 295 |
| 4-(9-methyl-2- (5-phenyl-1,2,4- oxadiazol-3-yl)- 8-(pyridin-4-yl)- 9H-purin-6- yl)morpholine | 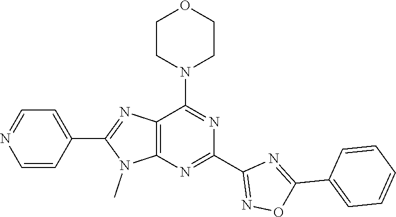 [SMILES: Cn1c(-c2ccncc2)nc2c(N3CCOCC3)nc(-c3noc(-c4ccccc4)n3)nc21] | ¹H NMR (400 MHz, Chloroform-d) δ 8.85- 8.80 (m, 2H), 8.34-8.26 (m, 2H), 7.81-7.73 (m, 2H), 7.66-7.52 (m, 3H), 4.38 (s, 4H), 4.10 (s, 3H), 3.94-3.86 (m, 4H); LCMS (ESI) m/z: 441.1 [M + H]⁺. | 296 |
| 4-(9-methyl-2- (5-phenyl-1,2,4- oxadiazol-3-yl)- 8-(tetrahydro- 2H-pyran-4-yl)- 9H-purin-6- yl)morpholine | 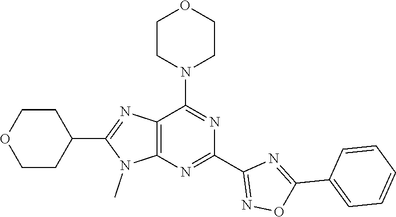 [SMILES: Cn1c(C2CCOCC2)nc2c(N3CCOCC3)nc(-c3noc(-c4ccccc4)n3)nc21] | ¹H NMR (400 MHz, DMSO-d₆) δ 8.20 (d, J = 7.2 Hz, 2H), 7.78-7.72 (m, 1H), 7.71-7.65 (m, 2H), 4.30 (bs, 4H), 3.98-3,95 (m, 2H), 3.81 (s, 3H), 3.77 (t, J = 4.8 Hz, 4H), 3.52 (dt, J = 11.1, 4.0 Hz, 2H), 3.32 (s, 1H), 1.92- 1.82 (m, 4H); LCMS (ESI) m/z: 448.2 [M + H]⁺. | 297 |

### Preparation of 3-cyclohexyl-1-[9-methyl-6-(morpholin-4-yl)-8-(pyridin-4-yl)-9H-purin-2-yl]-4,5-dihydro-1H-1,2,4-triazol-5-one (Compound 298)

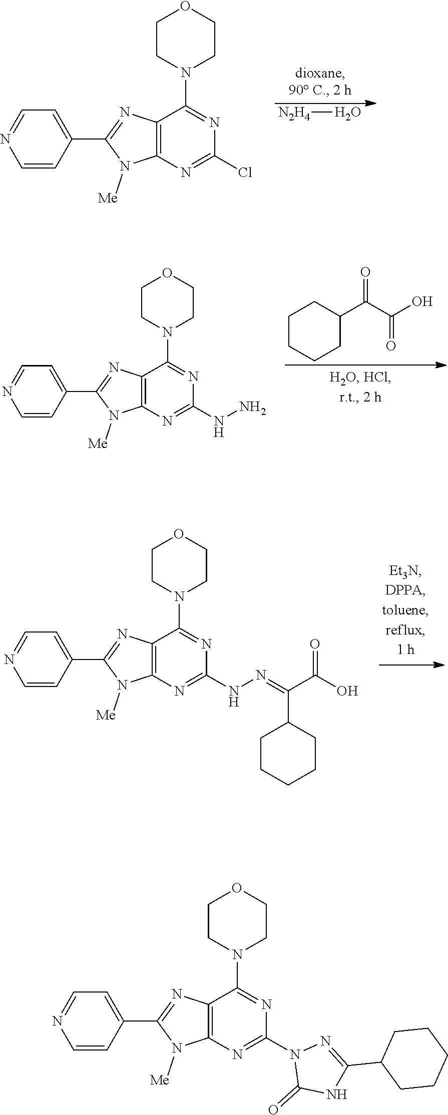

### Step 1: Preparation of 4-(2-hydrazinyl-9-methyl-8-(pyridin-4-yl)-9H-purin-6-yl)morpholine

To a solution of 4-(2-chloro-9-methyl-8-(pyridin-4-yl)-9H-purin-6-yl)morpholine (300 mg, 0.91 mmol) in dioxane (12 mL) was added hydrazine hydrate (3 mL) and the resultant mixture was heated to 90° C. and stirred for 2 h. It was concentrated and the crude product thus obtained was purified via flash column chromatography through silica gel using a gradient of 0-5% methanol in dichloromethane. The product 4-(2-hydrazinyl-9-methyl-8-(pyridin-4-yl)-9H-purin-6-yl)morpholine (283 mg, 0.87 mmol, 95%) was obtained as a white solid. LCMS (ESI) m/z: 327.1 [M+H]⁺.

### Step 2: Preparation of (E)-2-cyclohexyl-2-(2-(9-methyl-6-morpholino-8-(pyridin-4-yl)-9H-purin-2-yl)hydrazono)acetic acid

To a mixture of 4-(2-hydrazinyl-9-methyl-8-(pyridin-4-yl)-9H-purin-6-yl)morpholine (283 mg, 0.87 mmol) and 2-cyclohexyl-2-oxoacetic acid (271 mg, 1.7 mmol) in water (10 mL) was added concentrated hydrochloric acid (0.5 mL). The mixture was stirred at room temperature for 2 h. The resultant precipitate was filtered, washed with water and dried to obtain (E)-2-cyclohexyl-2-(2-(9-methyl-6-morpholino-8-(pyridin-4-yl)-9H-purin-2-yl)hydrazono)acetic acid (379 mg, 0.82 mmol, 94%) as a white solid. LCMS (ESI) m/z: 465.2 [M+H]⁺.

### Step 3: Preparation of 3-cyclohexyl-1-[9-methyl-6-(morpholin-4-yl)-8-(pyridin-4-yl)-9H-purin-2-yl]-4,5-dihydro-1H-1,2,4-triazol-5-one

To a solution of (E)-2-cyclohexyl-2-(2-(9-methyl-6-morpholino-8-(pyridin-4-yl)-9H-purin-2-yl)hydrazono)acetic acid (379 mg, 0.82 mmol) and triethylamine (165 mg, 1.6 mmol) in toluene (5 mL) was added diphenyl phosphorylazide (449 mg, 1.6 mmol). The resultant mixture was refluxed for 2 h and concentrated. The crude product was purified by prep-HPLC (SunFire C18, 4.6*50 mm, 3.5 um column Xbridge C18 3.5 μm 4.6×50 mm column. The elution system used was a gradient of 5%-95% over 1.5 min at 2 mL/min and the solvent was acetonitrile/0.01% aqueous ammonium bicarbonate) to obtain 3-cyclohexyl-1-[9-methyl-6-(morpholin-4-yl)-8-(pyridin-4-yl)-9H-purin-2-yl]-4,5-dihydro-1H-1,2,4-triazol-5-one (92.5 mg, 0.20 mmol, 25%) as a white solid. ¹H NMR (500 MHz, Chloroform-d) δ 11.97 (s, 1H), 8.80 (dd, J=4.5, 1.6 Hz, 2H), 7.75 (dd, J=4.5, 1.6 Hz, 2H), 4.58 (s, 4H), 4.00 (s, 3H), 3.88 (dd, J=11.7, 7.0 Hz, 4H), 2.75 (tt, J=11.9, 3.4 Hz, 1H), 2.13 (d, J=11.9 Hz, 2H), 1.91-1.82 (m, 2H), 1.78 (d, J=12.7 Hz, 1H), 1.59 (qd, J=12.6, 3.2 Hz, 2H), 1.39 (dt, J=12.8, 7.9 Hz, 2H), 1.28 (m, 1H); LCMS (ESI) m/z: 462.3 [M+H]⁺.

### Synthesis of 3-methyl-1-(9-methyl-6-morpholino-8-(pyridin-4-yl)-9H-purin-2-yl)-4-phenyl-1H-1,2,4-triazol-5(4H)-one (Compound 299)

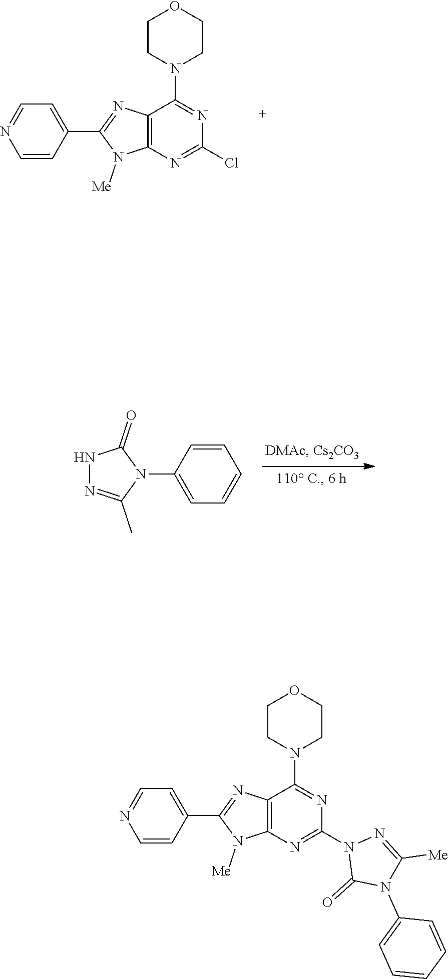 [SMILES: Cn1c(-c2ccncc2)nc2c(N3CCOCC3)nc(Cl)nc21 ; Cc1n[nH]c(=O)n1-c1ccccc1 ; Cc1nn(-c2nc(N3CCOCC3)c3nc(-c4ccncc4)n(C)c3n2)c(=O)n1-c1ccccc1]

To a stirred solution of 4-(2-chloro-9-methyl-8-(pyridin-4-yl)-9H-purin-6-yl)morpholine (179 mg, 0.543 mmol), 3-methyl-4-phenyl-1H-1,2,4-triazol-5(4H)-one (95 mg, 0.543 mmol) in N,N-dimethylacetamide (3 mL) was added cesium carbonate (531 mg, 1.629 mmol). The resultant mixture was heated to 120° C. and stirred for 6 h. It was concentrated and the resultant crude product was purified by HPLC (SunFire C18, 4.6*50 mm, 3.5 um column Xbridge C18 3.5 μm 4.6×50 mm column. The elution system used was a gradient of 5%-95% over 1.5 min at 2 ml/min and the solvent was acetonitrile/0.01% aqueous NH₄HCO₃) to afford 3-methyl-1-(9-methyl-6-morpholino-8-(pyridin-4-yl)-9H-purin-2-yl)-4-phenyl-1H-1,2,4-triazol-5(4H)-one (24.1 mg, 9.5%) as white solid.

¹H NMR (400 MHz, DMSO) δ 8.78 (dd, J=4.5, 1.6 Hz, 2H), 7.91 (dd, J=4.5, 1.6 Hz, 2H), 7.67-7.43 (m, 5H), 4.34 (bs, 4H), 3.91 (s, 3H), 3.76 (t, J=4.4 Hz, 4H), 2.17 (s, 3H); LCMS (ESI) m/z: 470.1 [M+H]+.

### Synthesis of 8-(9-ethyl-6-morpholino-2-(4-phenyl-1H-pyrazol-1-yl)-9H-purin-8-yl)octahydropyrazino[2,1-c][1,4]oxazine (Compound 300)

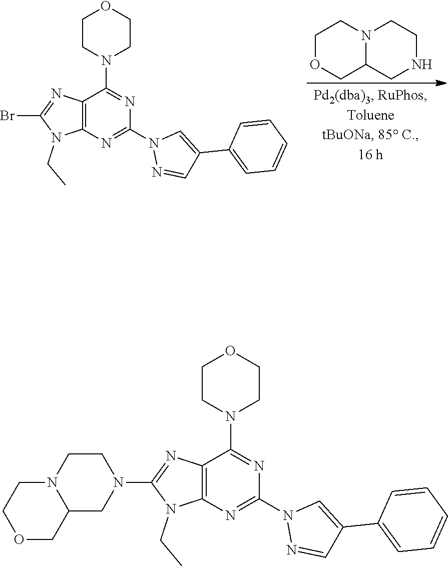 [SMILES: CCn1c(Br)nc2c(N3CCOCC3)nc(-n3cc(-c4ccccc4)cn3)nc21 ; C1CN2CCOCC2CN1 ; CCn1c(N2CCN3CCOCC3C2)nc2c(N3CCOCC3)nc(-n3cc(-c4ccccc4)cn3)nc21]

A mixture of 4-(8-bromo-9-ethyl-2-(4-phenyl-1H-pyrazol-1-yl)-9H-purin-6-yl)morpholine (200 mg, 0.44 mmol), octahydropyrazino [2,1-c][1,4]oxazine (90 mg, 0.66 mmol), tris(dibenzylideneacetone)dipalladium (40 mg, 0.044 mmol), 2-dicyclohexylphosphino-2′,6′-diisopropoxybiphenyl (20.5 mg, 0.044 mmol) and sodium tert-butoxide (429 mg, 1.32 mmol) in toluene (5 mL) was stirred at 85° C. under argon for 36 h. The mixture was filtered, and the filtrate was concentrated. The residue was purified by Prep-TLC (Dichloromethane/methanol=10/1) to give 8-(9-ethyl-6-morpholino-2-(4-phenyl-1H-pyrazol-1-yl)-9H-purin-8-yl)octahydropyrazino [2,1-c][1,4]oxazine (71 mg, 31%) as a white solid.

1H NMR (400 MHz, DMSO-d₆) δ 9.02 (d, J=0.7 Hz, 1H), 8.21 (d, J=0.8 Hz, 1H), 7.82-7.72 (m, 2H), 7.41 (t, J=7.7 Hz, 2H), 7.26 (t, J=7.4 Hz, 1H), 4.23 (s, 4H), 4.13 (q, J=7.2 Hz, 2H), 3.76 (t, J=8.5 Hz, 6H), 3.54 (d, J=2.1 Hz, 1H), 3.49 (s, 1H), 3.16 (t, J=10.5 Hz, 1H), 3.04 (d, J=2.5 Hz, 1H), 2.83 (d, J=11.3 Hz, 1H), 2.70-2.61 (m, 2H), 2.48-2.21 (m, 4H), 1.40 (t, J=7.2 Hz, 3H). LCMS (ESI) m/z: 516.2 [M+H]⁺.

The following compounds were synthesized according to the protocol described above:

| Name | Structure | NMR, MS | # |
|---|---|---|---|
| 4-(9-ethyl-6- morpholino-2-(4- phenyl-1H-pyrazol- 1-yl)-9H-purin-8-yl)- 1-methylpiperazin- 2-one | 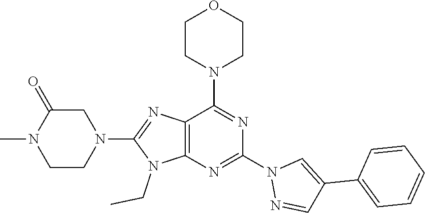 [SMILES: CCn1c(N2CCN(C)C(=O)C2)nc2c(N3CCOCC3)nc(-n3cc(-c4ccccc4)cn3)nc21] | 1H NMR (400 MHz, DMSO-d₆) δ 9.03 (s, 1H), 8.22 (s, 1H), 8.14 (s, 1H), 7.78 (d, J = 7.2 Hz, 2H), 7.41 (t, J = 7.7 Hz, 2H), 7.27 (t, J = 7.3 Hz, 1H), 4.24 (bs, 4H), 4.17 (q, J = 7.2 Hz, 2H), 3.88 (s, 2H), 3.81-3.69 (m, 4H), 3.51 (dd, J = 19.2, 5.4 Hz, 4H), 2.91 (s, 3H), 1.40 (t, J = 7.2 Hz, 3H); LCMS (ESI) m/z: 488.3 [M + H]⁺ | 301 |
| 1-(9-ethyl-6- morpholino-2-(4- phenyl-1H-pyrazol- 1-yl)-9H-purin-8-yl)- 4-methylpiperazin- 2-one | 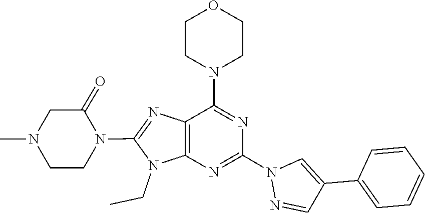 [SMILES: CCn1c(N2CCN(C)CC2=O)nc2c(N3CCOCC3)nc(-n3cc(-c4ccccc4)cn3)nc21] | 1H NMR (400 MHz, CDCl3) δ 8.77 (s, 1H), 8.08 (s, 1H), 7.61 (d, J = 7.4 Hz, 2H), 7.41 (t, J = 7.6 Hz, 2H), 7.26 (s, 1H), 4.33 (s, 4H), 4.20 (q, J = 6.8 Hz, 2H), 3.90-3.80 (s, 6H), 3.33 (s, 2H), 2.86 (t, J = 5.4 Hz, 2H), 2.44 (s, 3H), 1.46 (d, J = 7.3 Hz, 3H); LCMS (ESI) m/z: 487.8 [M+]⁺ | 302 |

### Synthesis of (S)-4-(9-ethyl-6-(3-methylmorpholino)-2-(4-phenyl-1H-pyrazol-1-yl)-9H-purin-8-yl)-1-methylpiperazin-2-one (Compound 303)

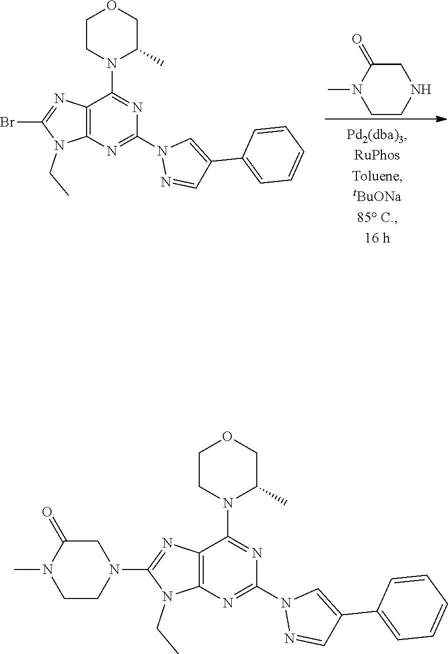

A mixture of (S)-4-(8-bromo-9-ethyl-2-(4-phenyl-1H-pyrazol-1-yl)-9H-purin-6-yl)-3-methylmorpholine (47 mg, 0.1 mmol), 1-methylpiperazin-2-one (17 mg, 0.15 mmol), tris(dibenzylideneacetone)dipalladium (9 mg, 0.01 mmol), 2-dicyclohexylphosphino-2′,6′-diisopropoxybiphenyl (9 mg, 0.01 mmol) and sodium tert-butoxide (29 mg, 0.3 mmol) in toluene (3 mL) was stirred at 85° C. for 16 h under argon. The reaction mixture was cooled, filtered and concentrated to obtain the crude product. It was purified by prep-HPLC (BOSTON pHlex ODS 10 μm 21.2×250 mm 120 A. The mobile phase was acetonitrile/0.1% Ammonium bicarbonate) to obtain (S)-4-(9-ethyl-6-(3-methylmorpholino)-2-(4-phenyl-1H-pyrazol-1-yl)-9H-purin-8-yl)-1-methylpiperazin-2-one (41.5 mg, 55%) as white solid.

H NMR (500 MHz, DMSO-d₆) δ 8.99 (s, 1H), 8.21 (d, J=0.4 Hz, 1H), 7.77 (d, J=7.2 Hz, 2H), 7.41 (t, J=7.7 Hz, 2H), 7.27 (t, J=7.4 Hz, 1H), 5.37 (s, 1H), 5.03 (s, 1H), 4.16 (q, J=7.2 Hz, 2H), 4.03-3.96 (m, 1H), 3.91-3.82 (m, 2H), 3.79 (d, J=11.4 Hz, 1H), 3.71 (dd, J=11.5, 2.9 Hz, 1H), 3.59-3.38 (m, 6H), 2.91 (s, 3H), 1.41 (t, J=7.2 Hz, 3H), 1.33 (d, J=6.7 Hz, 3H); LCMS: (ESI) m/z: 502.0 [M+H]⁺.

### Synthesis of (R)-4-(9-ethyl-6-(3-methylmorpholino)-2-(4-phenyl-1H-pyrazol-1-yl)-9H-purin-8-yl)-1-methylpiperazin-2-one (Compound 304)

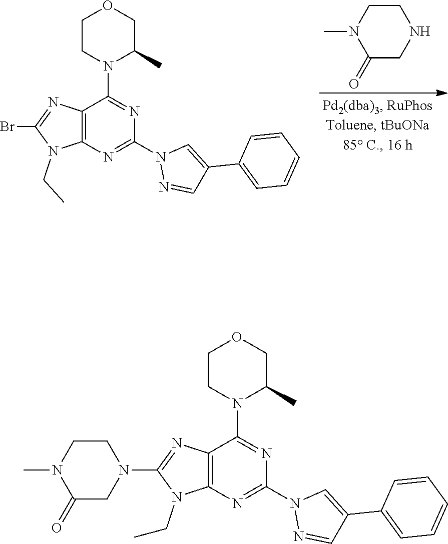

A mixture of (R)-4-(8-bromo-9-ethyl-2-(4-phenyl-1H-pyrazol-1-yl)-9H-purin-6-yl)-3-methylmorpholine (200 mg, 0.42 mmol), 1-methylpiperazin-2-one (91 mg, 0.64 mmol), dicyclohexyl(2′,6′-diisopropoxy-[1,1′-biphenyl]-2-yl)phosphane (39 mg, 0.08 mmol) and sodium tert-butoxide (82 mg, 0.85 mmol) in toluene (2 mL) was stirred at 85° C. for 16 h. It was concentrated and the resultant crude product was purified by silica gel chromatography (Dichloromethane/Methanol 20:1->10:1->5:1) to obtain (R)-4-(9-ethyl-6-(3-methylmorpholino)-2-(4-phenyl-1H-pyrazol-1-yl)-9H-purin-8-yl)-1-methylpiperazin-2-one (100 mg, 47%) as white solid.

¹H NMR (400 MHz, DMSO-d₆) δ 9.00 (s, 1H), 8.22 (s, 1H), 7.78 (d, J=7.2 Hz, 2H), 7.41 (t, J=7.7 Hz, 2H), 7.30 (t, J=8 Hz, 1H), 5.38 (bs, 1H), 5.04 (bs, 1H), 4.15 (t, J=7.2 Hz, 2H), 4.00 (d, J=8.3 Hz, 1H), 3.87 (s, 2H), 3.75-3.60 (m, 2H), 3.53 (d, J=4 Hz, 3H), 3.48 (d, J=4 Hz, 3H), 2.91 (s, 3H), 1.40 (t, J=7.2 Hz, 3H), 1.33 (d, J=6.7 Hz, 3H); LCMS (ESI) m/z: 502.2 [M+H]⁺

The following compounds were synthesized according to the protocol described earlier.

| Name | Structure | NMR, MS | # |
|---|---|---|---|
| 1-(9-ethyl-2-(3- methoxy-4-phenyl- 1H-pyrazol-1-yl)-6- morpholino-9H- purin-8-yl)-4- methylpiperazin-2- one | 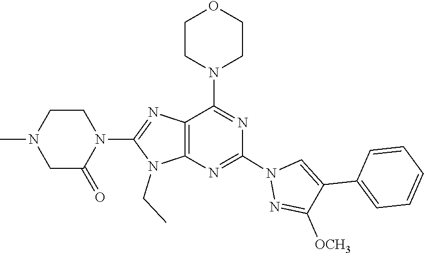 [SMILES: CCn1c(N2CCN(C)CC2=O)nc2c(N3CCOCC3)nc(-n3cc(-c4ccccc4)c(OC)n3)nc21] | 1H NMR (400 MHz, DMSO-d₆) δ 8.94 (s, 1H), 8.22 (s, 1H), 7.82 (d, J = 8.5 Hz, 2H), 7.35 (d, J = 8.5 Hz, 2H), 4.53-4.14 (m, 6H), 4.07 (s, 3H), 3.77 (d, J = 4.3 Hz, 4H), 3.67 (t, J = 5.2 Hz, 2H), 3.12 (s, 2H), 2.74 (s, 2H), 2.29 (s, 3H), 1.45 (t, J = 7.2 Hz, 3H); LCMS (ESI) m/z: 517.8[M + H]+. | 305 |
| 1-(9-ethyl-2-(3- methoxy-4-phenyl- 1H-pyrazol-1-yl)-6- morpholino-9H- purin-8-yl)piperidin- 4-ol | 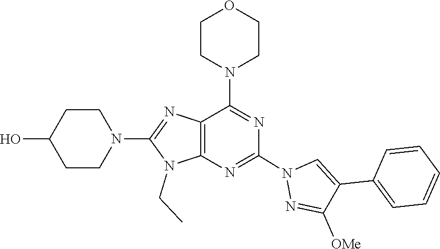 [SMILES: CCn1c(N2CCC(O)CC2)nc2c(N3CCOCC3)nc(-n3cc(-c4ccccc4)c(OC)n3)nc21] | ¹H NMR (400 MHz, DMSO-d₆) δ 8.88 (s, 1H), 7.87-7.73 (m, 2H), 7.39 (t, J = 7.8 Hz, 2H), 7.24 (t, J = 7.4 Hz, 1H), 4.77 (s, 1H), 4.22 (s, 4H), 4.10-4.08 (m, 2H), 4.06 (s, 3H), 3.82- 3.70 (m, 4H), 3.68 (s, 1H), 3.45-3.35 (m, 2H), 2.98 (t, J = 9.9 Hz, 2H), 1.89 (d, J = 9.1 Hz, 2H), 1.65-1.52 (m, 2H), 1.41 (t, J = 7.2 Hz, 3H); LCMS (ESI) m/z: 505.8 [M + H]⁺. | 306 |

### Synthesis of 4-(9-ethyl-2-(3-methoxy-4-phenyl-1H-pyrazol-1-yl)-6-morpholino-9H-purin-8-yl)-1-methylpiperazin-2-one (Compound 307)

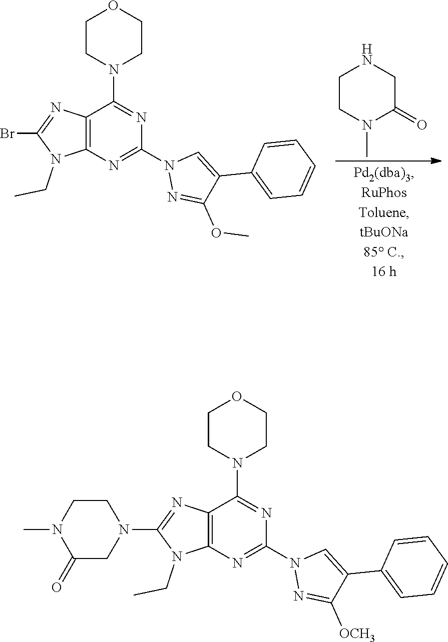

A mixture of 4-(8-bromo-9-ethyl-2-(3-methoxy-4-phenyl-1H-pyrazol-1-yl)-9H-purin-6-yl)morpholine (150 mg, 0.310 mmol), 1-methylpiperazin-2-one (71 mg, 0.620 mmol), tris(dibenzylideneacetone)dipalladium (30 mg, 0.05 mmol), 2-dicyclohexylphosphino-2′,6′-diisopropoxy-1,1′-biphenyl (56 mg, 0.06 mmol) and sodium 2-methylpropan-2-olate (60 mg, 0.620 mmol) in toluene (10 mL) was stirred at 85° C. for 16 h under microwave irradiation. The reaction mixture was cooled, quenched with water (15 mL) and extracted with ethyl acetate (20*3 mL). The organic layers were combined, washed with brine (30 mL), dried over sodium sulfate, filtered and concentrated. The residue was purified by prep-HPLC (0.05% NH4HCO3/H2O:CH3CN=5%-95%) to obtain 4-(9-ethyl-2-(3-methoxy-4-phenyl-1H-pyrazol-1-yl)-6-morpholino-9H-purin-8-yl)-1-methylpiperazin-2-one (16.7 mg, 40%) as white solid.

¹H NMR (400 MHz, DMSO-d₆) δ 8.89 (s, 1H), 7.82-7.80 (m, 2H), 7.39 (t, J=7.8 Hz, 2H), 7.24 (t, J=7.4 Hz, 1H), 4.48-4.11 (m, 6H), 4.06 (s, 3H), 3.86 (s, 2H), 3.80-3.68 (m, 4H), 3.50 (dd, J=15.0, 5.1 Hz, 4H), 2.91 (s, 3H), 1.39 (t, J=7.2 Hz, 3H); LCMS (ESI) m/z: 517.8[M+]+.

### Synthesis of 1-(9-ethyl-2-(3-(1-methyl-1H-pyrazol-3-yl)phenyl)-6-morpholino-9H-purin-8-yl)-4-methylpiperazin-2-one (Compound 308)

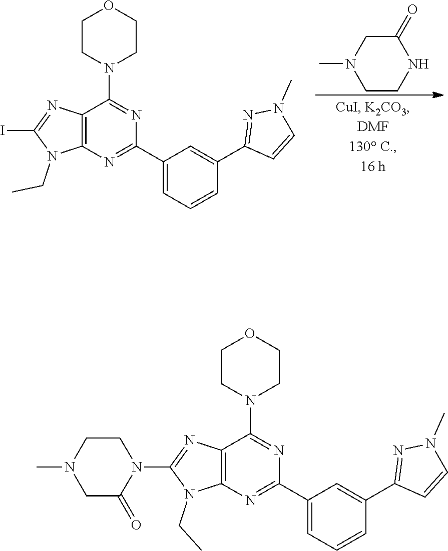

A mixture of 4-(9-ethyl-8-iodo-2-(3-(1-methyl-1H-pyrazol-3-yl)phenyl)-9H-purin-6-yl)morpholine (50 mg, 0.1 mmol), methylpiperazin-2-one (17 mg, 0.15 mmol), cuprous iodide (1.9 mg, 0.01 mmol) and potassium carbonate (27.6 mg, 0.2 mmol) in N,N-dimethylformamide (3 mL) was stirred at 130° C. under microwave for 16 h. The resultant mixture was cooled and filtered through a celite pad and the filtrate was concentrated to give the crude product. It was purified by prep-TLC (1%-5% methanolin in dichloromethane) to obtain 1-(9-ethyl-2-(3-(1-methyl-1H-pyrazol-3-yl)phenyl)-6-morpholino-9H-purin-8-yl)-4-methylpiperazin-2-one (30.9 mg, 60%) as white solid. 1H NMR (400 MHz, DMSO-d₆) δ 8.75 (d, J=1.6 Hz, 1H), 8.31 (d, J=7.9 Hz, 1H), 7.86 (d, J=7.8 Hz, 1H), 7.78 (d, J=2.2 Hz, 1H), 7.50 (t, J=7.8 Hz, 1H), 6.75 (d, J=2.2 Hz, 1H), 4.26 (s, 4H), 4.08 (q, J=7.0 Hz, 2H), 3.92 (s, 3H), 3.82-3.72 (m, 6H), 3.27 (s, 2H), 2.82 (s, 2H), 2.35 (s, 3H), 1.40 (t, J=7.2 Hz, 3H); LCMS (ESI) m/z: 502.2 [M+H]⁺.

### Synthesis of 4-(9-ethyl-2-(3-(1-methyl-1H-pyrazol-3-yl)phenyl)-6-morpholino-9H-purin-8-yl)-1-methylpiperazin-2-one (Compound 309)

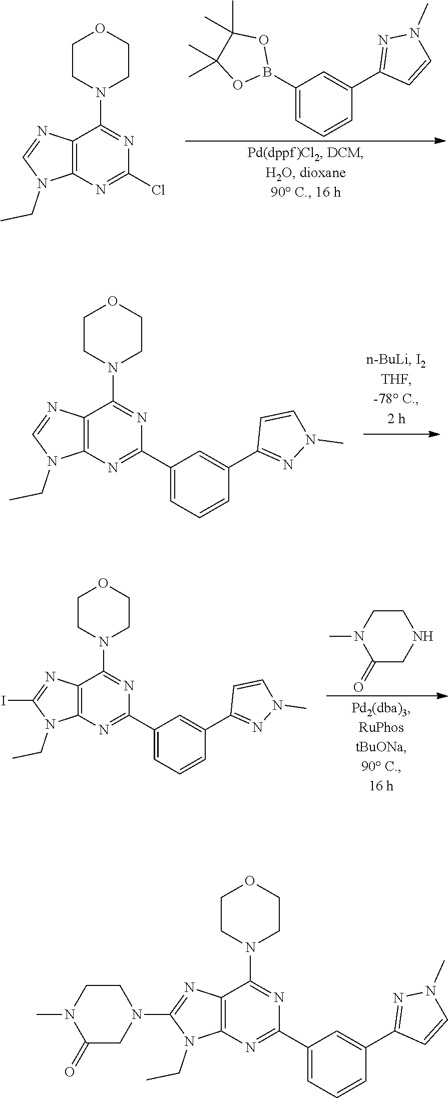

### Step 1: Preparation of 4-(9-ethyl-2-(3-(1-methyl-1H-pyrazol-3-yl)phenyl)-9H-purin-6-yl)morpholine

To a solution of 4-(2-chloro-9-ethyl-9H-purin-6-yl)morpholine (3.0 g, 11.0 mmol) in dioxane (60 mL) was added 1-methyl-3-(3-(4,4,5,5-tetramethyl-1,3,2-dioxaborolan-2-yl)phenyl)-1H-pyrazole (3.8 g, 14.0 mmol), [1,1′-bis(diphenylphosphino)ferrocene]dichloropalladium(II) (821.9 mg, 1.0 mmol) and cesium carbonate (10.9 g, 33.0 mmol) at 25° C. and the resulting mixture was stirred at 90° C. for 16 h under nitrogen protection. The mixture was then extracted with ethyl acetate (20 mL*2), washed with water (10 mL*2), dried over sodium sulfate and concentrated. The residue was purified by silica gel column chromatography (4% methanol in dichloromethane) to obtain 4-(9-ethyl-2-(3-(1-methyl-1H-pyrazol-3-yl)phenyl)-9H-purin-6-yl)morpholine as white solid. (3.2 g, 95.0%). LCMS (ESI) m/z: 390.3 [M+H]⁺.

### Step 2: Preparation of 4-(9-ethyl-8-iodo-2-(3-(1-methyl-1H-pyrazol-3-yl)phenyl)-9H-purin-6-yl)morpholine

To a solution of 4-(9-ethyl-2-(3-(1-methyl-1H-pyrazol-3-yl)phenyl)-9H-purin-6-yl)morpholine (2.0 g, 5.1 mmol) in tetrahydrofuran (20 mL) was added n-butyllithium (0.3 mL, 7.7 mmol) and iodine (1.57 g, 6.1 mmol at −78° C. and the reaction mixture was stirred at 25° C. for 2 h under nitrogen protection. The reaction was quenched by the addition of the saturated aqueous ammonium chloride and themixture was extracted with ethyl acetate (20 mL*2). The organic layer was washed with water (10 mL*2), dried over sodium sulfate and concentrated. The residue was purified by silica gel column chromatography (1% methanol in dichloromethane) to give the product 4-(9-ethyl-8-iodo-2-(3-(1-methyl-1H-pyrazol-3-yl)phenyl)-9H-purin-6-yl)morpholine as white solid (2.0 g, 75.7%). LCMS (ESI) m/z: 516.2 [M+H]⁺.

### Step 3: Preparation of 4-(9-ethyl-2-(3-(1-methyl-1H-pyrazol-3-yl)phenyl)-6-morpholino-9H-purin-8-yl)-1-methylpiperazin-2-one

To a solution of 4-(9-ethyl-8-iodo-2-(3-(1-methyl-1H-pyrazol-3-yl)phenyl)-9H-purin-6-yl)morpholine (200 mg, 0.38 mmol) in toluene (15 mL) were added 1-methylpiperazin-2-one (66.7 mg, 0.58 mmol), tris(dibenzylideneacetone)dipalladium(0) (17.6 mg, 0.04 mmol), sodium tert-butoxide (111.6 mg, 1.16 mmol) and 2-dicyclohexylphosphino-2′,6′-di-i-propoxy-1,1′-biphenyl (18.0 mg, 0.04 mmol) at 25° C. and the resultant reaction mixture was stirred at 90° C. for 16 h under nitrogen protection. The mixture was extracted with ethyl acetate (20 mL*2), washed with water (10 mL*2), dried over sodium sulfate, and concentrated. The residue was purified by silica gel column chromatography (5% methanol in dichloromethane) to give the product 4-(9-ethyl-2-(3-(1-methyl-1H-pyrazol-3-yl)phenyl)-6-morpholino-9H-purin-8-yl)-1-methylpiperazin-2-one as white solid (28 mg, 14.3%).

¹H NMR (400 MHz, DMSO-d₆) δ 8.73 (s, 1H), 8.28 (d, J=7.9 Hz, 1H), 7.83 (d, J=7.6 Hz, 1H), 7.77 (d, J=2.0 Hz, 1H), 7.48 (t, J=7.7 Hz, 1H), 6.74 (d, J=2.2 Hz, 1H), 4.43-4.04 (m, 6H), 3.91 (d, J=10.6 Hz, 5H), 3.85-3.67 (m, 4H), 3.63-3.39 (m, 4H), 2.91 (s, 3H), 1.44 (t, J=7.2 Hz, 3H); LCMS (ESI) m/z: 502.2 [M+H]⁺.

### Synthesis of 4-(2-(4-cyclobutyl-1H-pyrazol-1-yl)-9-ethyl-6-morpholino-9H-purin-8-yl)-1-methylpiperazin-2-one (Compound 310)

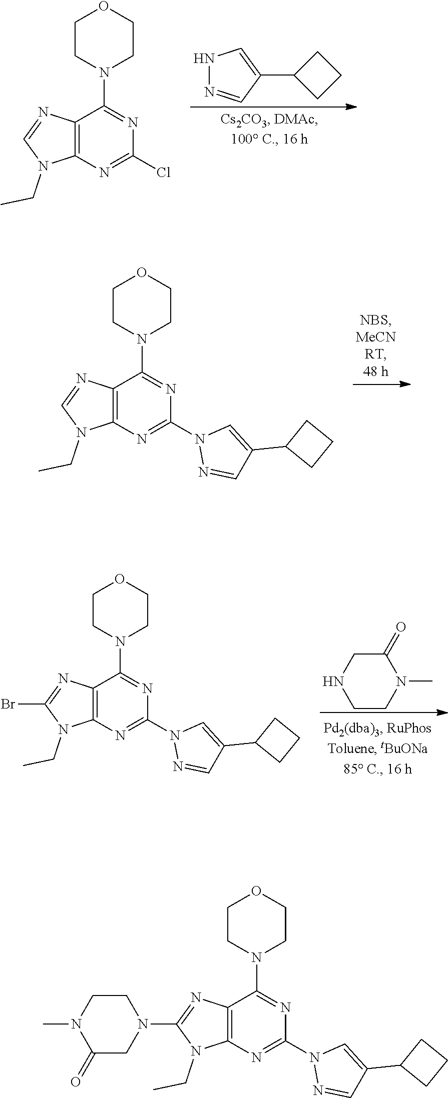

### Step 1: Synthesis of 4-(2-(4-cyclobutyl-1H-pyrazol-1-yl)-9-ethyl-9H-purin-6-yl)morpholine

A solution of 4-cyclobutyl-1H-pyrazole (143 mg, 0.87 mmol), 4-(2-chloro-9-ethyl-9H-purin-6-yl)morpholine (243 mg, 0.87 mmol) and cesium carbonate (1.04 g, 3.2 mmol) in dry N,N-dimethylaniline (15 mL) was stirred at 100° C. for 16 h. The reaction mixture was quenched with addition of saturated aqueous ammonium chloride (15 mL) and then diluted with water (30 mL) and extracted with ethyl acetate (50 mL*3). The organic layer was dried over sodium sulfate and the solvent was removed under reduced pressure. The resultant crude product was purified by silica gel chromatography eluting with a linear gradient of 0% to 25% ethyl acetate in petroleum ether to get 4-(2-(4-cyclobutyl-1H-pyrazol-1-yl)-9-ethyl-9H-purin-6-yl)morpholine (154 mg, 49.4%) as white solid. LCMS: [M+H]⁺=354.1.

### Step 2: Synthesis of 4-(8-bromo-2-(4-cyclobutyl-1H-pyrazol-1-yl)-9-ethyl-9H-purin-6-yl)morpholine

A solution of 4-(2-(4-cyclobutyl-1H-pyrazol-1-yl)-9-ethyl-9H-purin-6-yl)morpholine (154 mg, 0.43 mmol) and N-bromosuccinimide (116 mg, 65 mmol) in dry acetonitrile (6 mL) was stirred at room temperature for 48 h. The reaction mixture was concentrated, filtered and purified by prep-HPLC (BOSTON pHlex ODS 10 um 21.2×250 mm 120 A. The mobile phase was acetonitrile/0.1% Ammonium bicarbonate) to afford 4-(8-bromo-2-(4-cyclobutyl-1H-pyrazol-1-yl)-9-ethyl-9H-purin-6-yl)morpholine (28 mg, 14.2%) as white solid. LCMS: [M+H]⁺=431.7.

### Step 3: Synthesis of 4-(2-(4-cyclobutyl-1H-pyrazol-1-yl)-9-ethyl-6-morpholino-9H-purin-8-yl)-1-methylpiperazin-2-one

A solution of 4-(8-bromo-2-(4-cyclobutyl-1H-pyrazol-1-yl)-9-ethyl-9H-purin-6-yl)morpholine (34 mg, 0.08 mmol), 1-methylpiperazin-2-one (14 mg, 0.12 mmol), tris(dibenzylideneacetone)dipalladium (9 mg, 0.01 mmol), 2-dicyclohexyl phosphino-2′,6′-diisopropoxybiphenyl (9 mg, 0.01 mmol), and sodium tert-butoxide (23 mg, 0.24 mmol) in toluene (3 mL) was stirred at 85° C. for 16 h under argon. The reaction mixture was filtered, concentrated and purified by pre-HPLC (BOSTON pHlex ODS 10 μm 21.2×250 mm 120 A. The mobile phase was acetonitrile/0.1% Ammonium bicarbonate) to obtain 4-(2-(4-cyclobutyl-1H-pyrazol-1-yl)-9-ethyl-6-morpholino-9H-purin-8-yl)-1-methylpiperazin-2-one (23.8 mg, 63.75%) as white solid.

¹H NMR (400 MHz, DMSO) δ 8.39 (s, 1H), 7.65 (s, 1H), 4.29-4.05 (m, 6H), 3.85 (s, 2H), 3.78-3.70 (m, 4H), 3.54-3.41 (m, 5H), 2.90 (s, 3H), 2.35-2.19 (m, 2H), 2.10-1.98 (m, 2H), 1.96-1.81 (m, 2H), 1.37 (t, J=7.1 Hz, 3H); LCMS: [M+H]⁺=465.8.

### Synthesis of (R)-3-methyl-4-(7-((S)-3-methylmorpholino)-2-(1H-pyrazol-3-yl)thiazolo[5,4-d]pyrimidin-5-yl)morpholine (Compound 311)

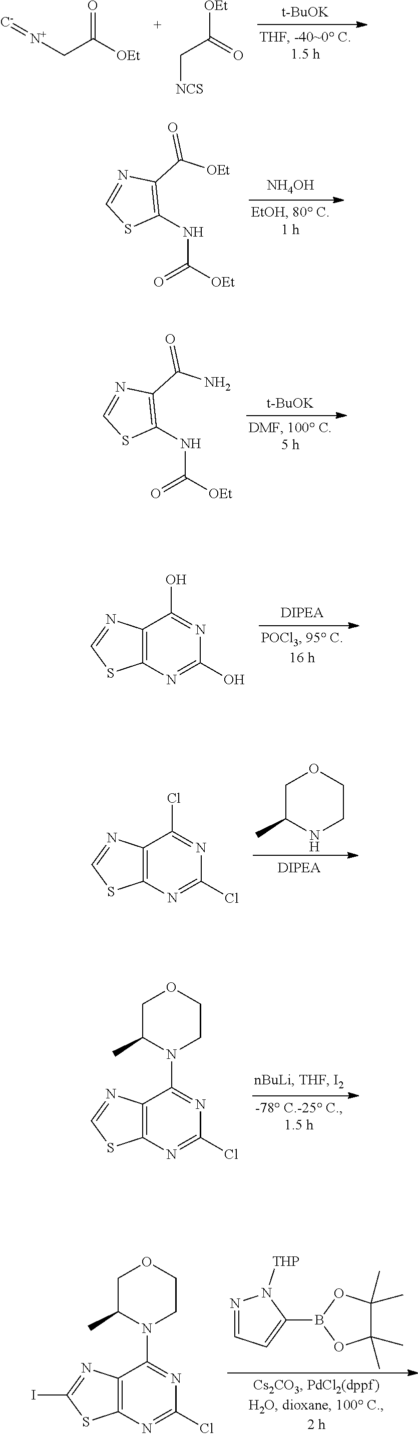

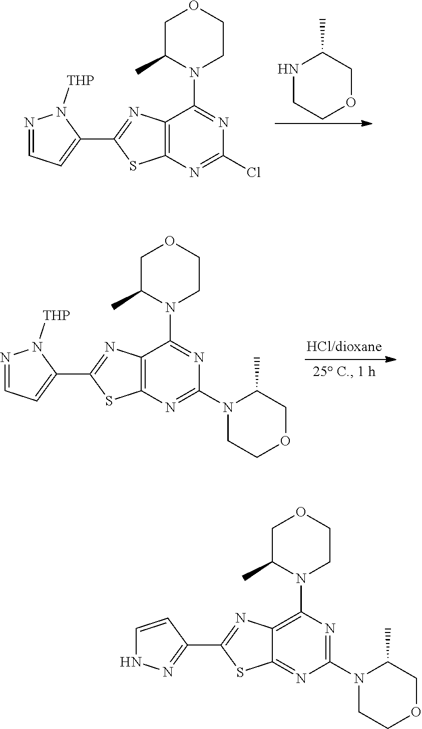

### Step 1: Preparation of ethyl 5-((ethoxycarbonyl)amino)thiazole-4-carboxylate

To a solution of potassium tert-butoxide in tetrahydrofuran (297.24 mL, 97.24 mmol) was added ethyl 2 isocyanoa-cetate (10 g, 88.4 mmol) drop wise at −40° C. followed by ethyl 2-isothiocyanatoacetate (13.48 g, 92.83 mmol). The resulting mixture was stirred for 1.5 h allowing the temperature to rise to 0° C. The reaction was quenched by addition of glacial acetic acid (50 mL) and the mixture was diluted with ethyl aceate (200 mL) and water (100 mL). The organics were separated and the aqueous layer was extracted with ethyl acetate (100 mL*2). The combined organic phase was washed with brine (50 mL) and dried over anhydrous sodium sulfate. The solution was concentrated and purified by flash column (acetic ester/petroleum ether 1:4) to give the desired product (11 g, 51%) as off-white solid. LCMS (ESI) m/z: 245.1 [M+H]⁺.

### Step 2: Preparation of ethyl (4-carbamoylthiazol-5-yl)carbamate

To a solution of ethyl 5-((ethoxycarbonyl)amino)thiazole-4-carboxylate (17 g, 69.6 mmol) in ethanol (17.0 mL) was added water (34 mL) and the mixture was stirred at 25° C. under for 10 minutes. To the mixture was added ammonium Hydroxide (120 ml) and the reaction was stirred at 80° C. for 1 h. After cooling to room temperature, the resulting solid was collected by filtration, rinsed with several portions of water, and dried in vacuo to give the desired product (10 g, 67%) as off-white solid. LCMS (ESI) m/z: 216.2 [M+H]⁺.

### Step 3: Preparation of thiazolo[5,4-d]pyrimidine-5,7-diol

To a solution of ethyl (4-carbamoylthiazol-5-yl)carbamate (8.0 g, 28.7 mmol) in DMF (100.0 mL) was added potassium tert-butoxide (71.7 mL, 71.7 mmol) and the resultant mixture was stirred at 100° C. under nitrogen for 2 h. The reaction was cooled to room temperature and filtered. The residue was rinsed with water (30 mL) and dried in vacuo to obtain the desired product as an off-white solid (5 g, 71%). LCMS (ESI) m/z: 170.1 [M+H]⁺.

### Step 4: Preparation of 5,7-dichlorothiazolo[5,4-d]pyrimidine

To a solution of thiazolo[5,4-d]pyrimidine-5,7-diol (5 g, 29.6 mmol) in phosphorus oxychloride (30 mL) was added DIPEA (4.0 mL). The reaction mixture was stirred at 100° C. for 15 h. The volatiles were evaporated and the residue was dissolved in ethyl acetate (100 mL) and washed with aqueous sodium bicarbonate (50 mL). The organics were dried and evaporated to give the desired product (4 g, 66%) as a yellow solid. LCMS (ESI) m/z: 206.1 [M+H]⁺.

### Step 5: Preparation of (S)-4-(5-chlorothiazolo[5,4-d]pyrimidin-7-yl)-3-methylmorpholine

To a mixture of 5,7-dichlorothiazolo[5,4-d]pyrimidine (2.00 g, 9.71 mmol) and DIPEA (3.40 mL, 19.4 mmol) in isopropanol (2.9 mL) was added dropwise (S)-3-methylmorpholine (1.08 g, 10.7 mmol) and the resulting mixture was stirred at 25° C. for 1.5 h. The mixture was concentrated and the residue was triturated with water, the solid formed was collected by filtration. The compound was washed with water (3×10 mL) and dried under high vacuum to afford the desired product (2.10 g, 80%) as a brown solid. LCMS (ESI) m/z: 271.1 [M+H]⁺.

### Step 6: Preparation of (S)-4-(5-chloro-2-iodothiazolo[5,4-d]pyrimidin-7-yl)-3-methylmorpholine

To a solution of (S)-4-(5-chlorothiazolo[5,4-d]pyrimidin-7-yl)-3-methylmorpholine (1.2 g, 4.43 mmol) in tetrahydrofuran (50 ml) was added n-butyllithium (3.4 ml, 8.5 mmol) at −78° C. and stirred at −78° C. for 0.5 h. Then a solution of iodine (2.25 g, 8.86 mmol) in tetrahydrofuran (10 ml) was added to the reaction mixture and stirred further at −78° C.-25° C. for 2 h. The reaction was quenched with saturated sodium thiosulfate solution, extracted with ethyl acetate (100 mL*2). The combined organic phase was washed with brine (100 mL), dried, concentrated. The resultant residue was slurried in EtOAc/DCM (15 mL, v/v=10:1) mixture to afford (S)-4-(5-chloro-2-iodothiazolo[5,4-d]pyrimidin-7-yl)-3-methylmorpholine as yellow solid (1 g, 57%). LCMS (ESI) m/z: 396.9/398.9 [M+]⁺.

### Step 7: Preparation of (3S)-4-(5-chloro-2-(1-(tetrahydro-2H-pyran-2-yl)-1H-pyrazol-5-yl)thiazolo[5,4-d]pyrimidin-7-yl)-3-methylmorpholine

To a solution of (S)-4-(5-chloro-2-iodothiazolo[5,4-d]pyrimidin-7-yl)-3-methylmorpholine (0.6 g, 1.5 mmol) in dioxane/water (12 mL/4 mL) was added 1-(tetrahydro-2H-pyran-2-yl)-5-(4,4,5,5-tetramethyl-1,3,2-dioxaborolan-2-yl)-1H-pyrazole (0.63 g, 2.3 mmol), cesium carbonate (0.985 g, 3.0 mmol) and [1,1′-bis(diphenylphosphino)ferrocene]dichloropalladium(II) (0.11 g, 0.15 mmol) at 25° C. and the reaction mixture was stirred at 100° C. for 4 h under argon atmosphere. The resultant mixture was filtered and purified by silica gel column (dichloromethane:acetic ester=10:1) to give the desired product as white solid (0.14 g, 22%) LCMS (ESI) m/z: 421.2 [M+H]⁺.

### Step 8: Preparation of (3R)-3-methyl-4-(7-((S)-3-methylmorpholino)-2-(1-(tetrahydro-2H-pyran-2-yl)-1H-pyrazol-5-yl)thiazolo[5,4-d]pyrimidin-5-yl)morpholine

To a solution of (3S)-4-(5-chloro-2-(1-(tetrahydro-2H-pyran-2-yl)-1H-pyrazol-5-yl)thiazolo[5,4-d]pyrimidin-7-yl)-3-methyl morpholine (0.1 g, 0.24 mmol) in DMAC (3 mL) were added (R)-3-methylmorpholine (0.048 g, 0.48 mmol) and cesium carbonate (0.232 g, 0.72 mmol) at 25° C. and the reaction mixture was stirred at 100° C. for 16 h under argon atmosphere. It was cooled, and filtered and the filtrate was purified by silica gel column (dichloromethane:acetic ester=10:1) to obtain the desired product as a white solid (0.07 g, 61%) LCMS (ESI) m/z: 486.2 [M+H]⁺.

### Step 9: Preparation of (R)-3-methyl-4-(7-((S)-3-methylmorpholino)-2-(1H-pyrazol-3-yl)thiazolo[5,4-d]pyrimidin-5-yl)morpholine

A solution of ((3R)-3-methyl-4-(7-((S)-3-methylmorpholino)-2-(1-(tetrahydro-2H-pyran-2-yl)-1H-pyrazol-5-yl)thiazolo[5,4-d]pyrimidin-5-yl)morpholine (0.07 g, 0.14 mmol) in dioxane/hydrochloric acid (3 mL) was stirred at 25° C. for 1 h. The mixture was concentrated and purified by Prep-HPLC (SunFire C18, 4.6*50 mm, 3.5 um column Xbridge C18 3.5 μm 4.6×50 mm column. The mobile phase was acetonitrile/10 mM formic acid aqueous solution) to obtain the desired product as off-white solid (0.0192 g, 33%,). ¹H NMR (400 MHz, DMSO-d₆) δ 13.26 (s, 1H), 7.90 (s, 1H), 6.79 (d, J=2.1 Hz, 1H), 5.32 (s, 2H), 4.58 (d, J=4.1 Hz, 1H), 4.20 (d, J=11.5 Hz, 1H), 4.01-3.85 (m, 2H), 3.75-3.60 (m, 3H), 3.62-3.51 (m, 2H), 3.48-3.39 (m, 2H), 3.14 (dt, J=12.9, 3.6 Hz, 1H), 1.31 (d, J=6.7 Hz, 3H), 1.19 (d, J=6.7 Hz, 3H); LCMS (ESI) m/z: 402.2 [M+H]⁺.

The following compounds were synthesized according to the protocol described above:

| Name | Structure | NMR, MS | # |
|---|---|---|---|
| 4-(7-morpholino- 2-(pyridin-4- yl)thiazolo[5,4- d]pyrimidin-5-yl)- 2- phenylmorpholine | 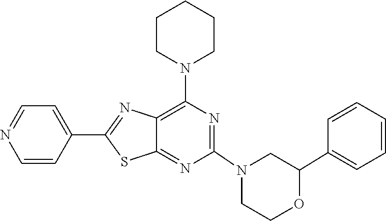 [SMILES: c1ccc(C2CN(c3nc(N4CCCCC4)c4nc(-c5ccncc5)sc4n3)CCO2)cc1] | ¹H NMR (400 MHz, DMSO-d₆) δ 8.71 (dd, J = 4.6, 1.5 Hz, 2H), 7.86 (dd, J = 4.6, 1.6 Hz, 2H), 7.45 (d, J = 7.0 Hz, 2H), 7.40 (t, J = 7.3 Hz, 2H), 7.34 (t, J = 7.1 Hz, 1H), 4.59 (d, J = 13.7 Hz, 1H), 4.52 (d, J = 10.7 Hz, 2H), 4.26 (s, 4H), 4.08 (d, J = 9.0 Hz, 1H), 3.82-3.73 (m, 4H), 3.68 (dd, J = 11.7, 9.1 Hz, 1H), 3.19-3.07 (m, 1H), 3.00-2.87 (m, 1H); LCMS (ESI) m/z: 461.2 [M + H]⁺. | 312 |

### Synthesis of (4-(9-ethyl-2-(4-phenyl-1H-pyrazol-1-yl)-8-(pyridin-4-yl)-9H-purin-6-yl)morpholin-3-yl)methanol (Compound 313)

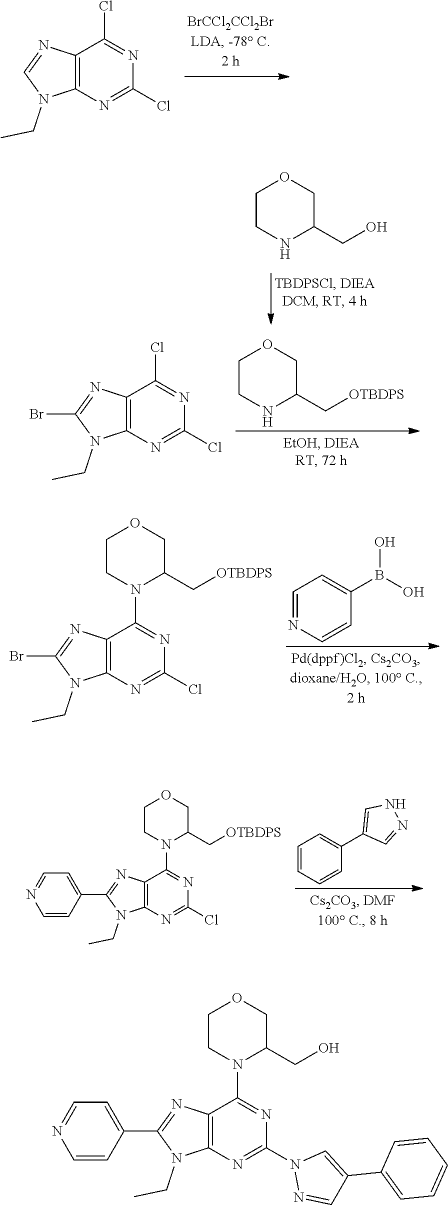

### Step 1a: Preparation of 3-((tert-butyldiphenylsilyloxy)methyl)morpholine

To a solution of morpholin-3-ylmethanol (500 mg, 4.27 mmol) and t-butylchlorodiphenylsilane (1.2 mL, 4.58 mmol) in dichloromethane (15 mL) was added N,N-diisopropylethylamine (930 mg, 7.21 mmol). The resultant mixture was stirred at 20° C. for 4 h and concentrated under reduced pressure. The residue was purified by flash chromatography (dichloromethane:methanol=50:1) to give 3-((tert-butyldiphenylsilyloxy)methyl)morpholine (500 mg, 33%) as colorless oil. LCMS (ESI) m/z: 356.2 [M+H]⁺.

### Step 1: Preparation of 8-bromo-2,6-dichloro-9-ethyl-9H-purine

To a solution of 2,6-dichloro-9-ethyl-9H-purine (50 mg, 0.23 mmol) in tetrahydrofuran (5 ml) was added lithium diisopropylamide (0.17 mL, 0.34 mmol) at −70° C. and the mixture was stirred at −70° C. for 1 h. Then 1,2-dibromotetrachloroethane (150 mg, 0.46 mmol) was added to the reaction mixture at −70° C., and the resultant mixture was stirred further at −70° C. for 1 h. Then the mixture was quenched by addition with water (10 mL), filtered and the filtrate was extracted with dichloromethane (10 ml*3). The organic layer was dried over sodium sulphate, filtered and concentrated under the reduced pressure. The residue was purified by flash chromatography on silica gel (petroleum ether:ethyl acetate=4:1) to give 8-bromo-2,6-dichloro-9-ethyl-9H-purine (50 mg, 74%) as yellow solid. LCMS (ESI) m/z: 296.9 [M+H]⁺.

### Step 2: Preparation of 4-(8-bromo-2-chloro-9-ethyl-9H-purin-6-yl)-3-((tert-butyldiphenylsilyloxy) methyl)morpholine

To a stirred solution of 8-bromo-2,6-dichloro-9-ethyl-9H-purine (50 mg, 0.17 mmol), 3-((tert-butyldiphenylsilyloxy)methyl)morpholine (90 mg, 0.25 mmol) in ethanol (5 mL) was added N,N-diisopropylethylamine (44 mg, 0.34 mmol). Then the reaction mixture was stirred at 20° C. for 16 h. It was concentrated under reduced pressure and the residue was purified by flash chromatography (dichloromethane:methanol=50:1) to give 4-(8-bromo-2-chloro-9-ethyl-9H-purin-6-yl)-3-((tert-butyldiphenylsilyloxy)methyl)morpholine (60 mg, 57%) as white solid. LCMS (ESI) m/z: 616.0 [M+H]⁺.

### Step 3: Preparation of 3-((tert-butyldiphenylsilyloxy)methyl)-4-(2-chloro-9-ethyl-8-(pyridin-4-yl)-9H-purin-6-yl)morpholine

A solution of 4-(8-bromo-2-chloro-9-ethyl-9H-purin-6-yl)-3-((tert-butyldiphenylsilyloxy)methyl)morpholine (60 mg, 0.098 mmol), pyridin-4-ylboronic acid (14 mg, 0.12 mmol), 1,1′-bis(diphenylphosphino) ferrocene-palladium(II) dichloride (4 mg, 0.005 mmol) and cesium carbonate (64 mg, 0.196 mmol) in dioxane/water (5.0 mL/1.0 mL) was stirred at 100° C. for 2 h under argon. The resultant mixture was diluted with ethyl acetate (25 mL) and washed with water (25 mL). The organic layer was dried over sodium sulphate, filtered and concentrated under the reduced pressure. The residue was purified by flash chromatography on silica gel (petroleum ether:ethyl acetate=1:1) to give 3-((tert-butyldiphenylsilyloxy)methyl)-4-(2-chloro-9-ethyl-8-(pyridin-4-yl)-9H-purin-6-yl)morpholine (50 mg, 83%) as yellow solid. LCMS (ESI) m/z: 613.2 [M+H]⁺.

### Step 4: Preparation of (4-(9-ethyl-2-(4-phenyl-1H-pyrazol-1-yl)-8-(pyridin-4-yl)-9H-purin-6-yl)morpholin-3-yl)methanol

A mixture of 3-((tert-butyldiphenylsilyloxy)methyl)-4-(2-chloro-9-ethyl-8-(pyridin-4-yl)-9H-purin-6-yl)morpholine (50 mg, 0.082 mmol), 4-phenyl-1H-pyrazole (14 mg, 0.098 mmol), cesium carbonate (53 mg, 0.164 mmol) in N,N-dimethylacetamide (5 mL) was stirred at 100° C. for 8 h. The resultant mixture was diluted with ethyl acetate (25 mL) and washed with water (25 mL). The organic layer was dried over sodium sulphate, filtered and concentrated under the reduced pressure. The crude product obtained was purified by HPLC (BOSTON pHlex ODS 10 μm 21.2×250 mm 120 A. The mobile phase was acetonitrile/0.1% Ammonium bicarbonate) to obtain (4-(9-ethyl-2-(4-phenyl-1H-pyrazol-1-yl)-8-(pyridin-4-yl)-9H-purin-6-yl)morpholin-3-yl)methanol (13.8 mg, 35%) as a white solid.

¹H NMR (400 MHz, DMSO-d₆) δ 9.08 (s, 1H), 8.81 (d, d, J=4.6, 1.5 Hz, 2H), 8.27 (d, J=0.7 Hz, 1H), 7.87 (d, J=4.9 Hz, 2H), 7.80 (d, J=7.4 Hz, 2H), 7.43 (t, J=7.7 Hz, 2H), 7.29 (t, J=7.4 Hz, 1H), 5.82 (s, 1H), 5.02 (s, 2H), 4.45 (q, J=7.1 Hz, 2H), 4.11 (s, 1H), 4.04-3.80 (m, 2H), 3.61-3.59 (m, 4H), 1.36 (t, J=7.2 Hz, 3H); LCMS (ESI) m/z: 483.1 [M+H]⁺.

### Synthesis of 9-ethyl-6-morpholino-8-(pyridin-4-yl)-N-(m-tolyl)-9H-purine-2-carboxamide (Compound 314)

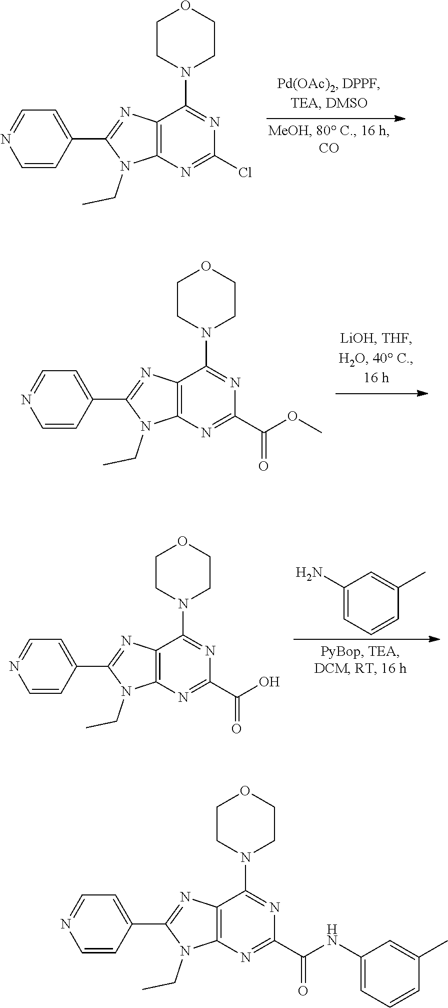

### Step 1: Preparation of methyl 9-ethyl-6-morpholino-8-(pyridin-4-yl)-9H-purine-2-carboxylate

To a solution of 4-(2-chloro-9-ethyl-8-(pyridin-4-yl)-9H-purin-6-yl)morpholine (350 mg, 1.01 mmol) in dimethyl sulfoxide (9 mL) and methanol (5 mL) were added palladium (II) acetate (24.6 mg, 0.11 mmol), 1,1′-bis(diphenylphosphino)ferrocene (122 mg, 0.22 mmol) and triethylamine (333 mg, 3.03 mmol) and the reaction mixture was stirred at 80° C. for 16 h under nitrogen atmosphere. The mixture was extracted with ethyl acetate (20 mL*2) and washed with water (10 mL*2). The organic layer was dried over sodium sulfate, and concentrated. The residue thus obtained was purified by silica gel column chromatography (3% methanol in in dichloromethane) to give the product methyl 9-ethyl-6-morpholino-8-(pyridin-4-yl)-9H-purine-2-carboxylate as white solid (100 mg, 27.2%). LCMS (ESI) m/z: 369.0 [M+H]⁺.

### Step 2: Preparation of 9-ethyl-6-morpholino-8-(pyridin-4-yl)-9H-purine-2-carboxylic acid

To a solution of methyl 9-ethyl-6-morpholino-8-(pyridin-4-yl)-9H-purine-2-carboxylate (100 mg, 0.27 mmol) in tetrahydrofuran (10 mL) and water (2 mL) was added lithium hydroxide (64.7 mg, 2.70 mmol, the reaction mixture was stirred at 40° C. for 16 h under nitrogen protection. The mixture was adjusted to pH=3 with hydrochloric acid, the precipitate thus formed was filtered to give the crude product 9-ethyl-6-morpholino-8-(pyridin-4-yl)-9H-purine-2-carboxylic acid as white solid (90 mg, 94.1%). LCMS (ESI) m/z: 355.0 [M+H]⁺.

### Step 3: Preparation of 9-ethyl-6-morpholino-8-(pyridin-4-yl)-N-(m-tolyl)-9H-purine-2-carboxamide

To a solution of 9-ethyl-6-morpholino-8-(pyridin-4-yl)-9H-purine-2-carboxylic acid (60 mg, 0.17 mmol) in dichloromethane (10 mL) were added m-toluidine (27.4 mg, 0.25 mmol), benzotriazole-1-yl-oxytripyrrolidinophosphonium hexafluorophosphate (115 mg, 0.22 mmol) and triethylamine (51.5 mg, 0.51 mmol). The resultant mixture was stirred at 25° C. for 16 h under nitrogen protection and concentrated. The crude product was purified by pre-HPLC (BOSTON pHlex ODS 10 μm 21.2×250 mm 120 A. The mobile phase was acetonitrile/0.1% Ammonium bicarbonate) to give the desired product 9-ethyl-6-morpholino-8-(pyridin-4-yl)-N-(m-tolyl)-9H-purine-2-carboxamide as white solid (51.0 mg, 67.7%). ¹H NMR (400 MHz, DMSO-d₆) δ 10.31 (s, 1H), 8.82 (d, J=5.0 Hz, 2H), 7.88 (d, J=5.1 Hz, 2H), 7.71-7.60 (m, 2H), 7.27 (t, J=7.8 Hz, 1H), 6.97 (d, J=7.5 Hz, 1H), 4.51 (q, J=7.2 Hz, 2H), 4.48 (bs, 4H), 3.87-3.73 (m, 4H), 2.34 (s, 3H), 1.33 (t, J=7.2 Hz, 3H). LCMS (ESI) m/z: 443.9 [M+H]⁺.

The following compounds were synthesized according to the protocol described above:

| Name | Structure | NMR, MS | # |
|---|---|---|---|
| 9-ethyl-N-(3- methylbenzyl)-6- morpholino-8- (pyridin-4-yl)-9H- purine-2- carboxamide | 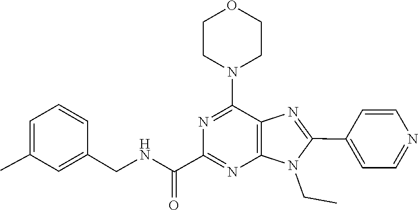 [SMILES: CCn1c(-c2ccncc2)nc2c(N3CCOCC3)nc(C(=O)NCc3cccc(C)c3)nc21] | ¹H NMR (400 MHz, DMSO-d₆) δ 9.24 (t, J = 6.4 Hz, 1H), 8.81 (dd, J = 4.5, 1.6 Hz, 2H), 7.85 (dd, J = 4.5, 1.6 Hz, 2H), 7.22 (t, J = 7.5 Hz, 1H), 7.17-7.11 (m, 2H), 7.06 (d, J = 7.4 Hz, 1H), 4.78-3.90 (m, 8H), 3.83-3.69 (m, 4H), 2.29 (s, 3H), 1.31 (t, J = 7.2 Hz, 3H); LC-MS: [M+]⁺ = 457.8. | 315 |
| 9-ethyl-N-(4- methylpyridin-2- yl)-6-morpholino- 8-(pyridin-4-yl)- 9H-purine-2- carboxamide hydrochloride | 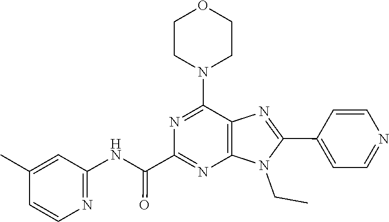 [SMILES: CCn1c(-c2ccncc2)nc2c(N3CCOCC3)nc(C(=O)Nc3cc(C)ccn3)nc21] | ¹H NMR (400 MHz, CD₃OD) δ 9.04 (d, J = 6.7 Hz, 2H), 8.66 (d, J = 6.8 Hz, 2H), 8.41 (d, J = 6.3 Hz, 1H), 8.05 (s, 1H), 7.58 (d, J = 5.5 Hz, 1H), 4.80-4.20 (m, 6H), 3.97-3.81 (m, 4H), 2.68 (s, 3H), 1.54 (t, J = 7.2 Hz, 3H); LCMS (ESI) m/z: 445.0 [M + H]⁺. | 316 |
| 9-ethyl-N-methyl- 6-morpholino-8- (pyridin-4-yl)-N- (m-tolyl)-9H- purine-2- carboxamide | 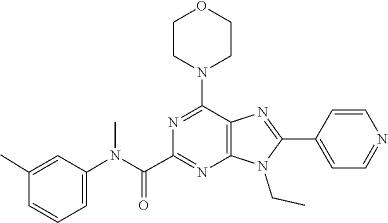 [SMILES: CCn1c(-c2ccncc2)nc2c(N3CCOCC3)nc(C(=O)N(C)c3cccc(C)c3)nc21] | ¹H NMR (400 MHz, DMSO-d₆) δ 8.77 (d, J = 5.4 Hz, 2H), 7.78 (s, 2H), 7.11-6.86 (m, 4H), 4.27 (s, 2H), 4.04 (bs, 4H), 3.61 (s, 4H), 3.36 (s, 3H), 2.19 (s, 3H), 1.19 (s, 3H); LCMS (ESI) m/z: 458.0 [M + H]⁺. | 317 |
| (3,4- dihydroisoquinolin- 2(1H)-yl)(9- ethyl-6- morpholino-8- (pyridin-4-yl)-9H- purin-2- yl)methanone | 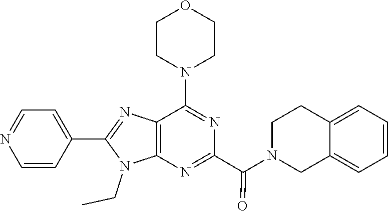 [SMILES: CCn1c(-c2ccncc2)nc2c(N3CCOCC3)nc(C(=O)N3CCc4ccccc4C3)nc21] | 1H NMR (400 MHz, DMSO-d₆) δ 8.81 (d, J = 5.5 Hz, 2H), 7.84 (d, J = 4.4 Hz, 2H), 7.45-6.96 (m, 4H),4.91-3.95 (m, 8H), 3.85 (t, J = 5.9 Hz, 0.5H), 3.83-3.66 (m, 4H), 3.48 (t, J = 5.5 Hz, 1.5H), 2.90- 2.75 (m, 2H), 1.40-1.20(m, 3H); LC-MS: [M + H]+ = 469.8. | 318 |
| 9 (9-ethyl-6- morpholino-8- (pyridin-4-yl)-9H- purin-2-yl)(4- methoxypiperidin- 1-yl)methanone | 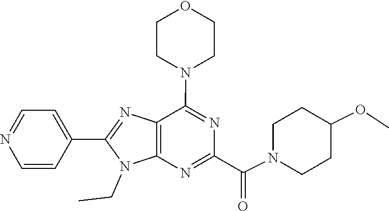 | 1H NMR (400 MHz, DMSO-d₆) δ 8.80 (dd, J = 4.5, 1.5 Hz, 2H), 7.82 (dd, J = 4.5, 1.5 Hz, 2H), 4.35 (q, J = 7.1 Hz, 6H), 3.96-3.89 (m, 1H), 3.81-3.69 (m, 4H), 3.49-3.43 (m, 1H), 3.29 (d, J = 5.0 Hz, 2H), 3.26 (s, 3H), 3.09-3.00 (m, 1H), 1.90 (s, 1H), 1.79 (s, 1H), 1.55-1.37 (m, 2H), 1.30 (t, J = 7.2 Hz, 3H); LC-MS: [M + H]⁺ = 451.8. | 319 |
| 9-cyclopropyl-6- morpholino-8- (pyridin-4-yl)-N- (m-tolyl)-9H- purine-2- carboxamide | 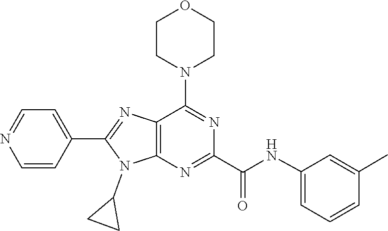 [SMILES: Cc1cccc(NC(=O)c2nc(N3CCOCC3)c3nc(-c4ccncc4)n(C4CC4)c3n2)c1] | 1H NMR (400 MHz, DMSO-d₆) δ 10.24 (s, 1H), 8.80 (d, J = 6.0 Hz, 2H), 8.13-7.95 (m, 2H), 7.70-7.55 (m, 2H), 7.28 (t, J = 7.7 Hz, 1H), 6.97 (d, J = 7.5 Hz, 1H), 4.37 (bs, 4H), 3.86-3.75 (m, 5H), 2.34 (s, 3H), 1.17 (t, J = 6.8 Hz, 2H), 0.85 (s, 2H); LCMS (ESI) m/z: 456.1 [M + H]⁺ | 320 |
| (4- cyclopropylpiperazin- 1-yl)(9-ethyl- 6-morpholino-8- (pyridin-4-yl)-9H- purin-2- yl)methanone | 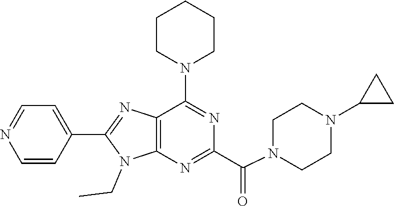 [SMILES: CCn1c(-c2ccncc2)nc2c(N3CCCCC3)nc(C(=O)N3CCN(C4CC4)CC3)nc21] | ¹H NMR (400 MHz, CDCl₃) δ 8.81 (d, J = 5.9 Hz, 2H), 7.68 (dd, J = 4.5, 1.6 Hz, 2H), 4.42 (q, J = 7.2 Hz, 6H), 3.94-3.82 (m, 4H), 3.82-3.75 (m, 2H), 3.43-3.31 (m, 2H), 2.84-2.70 (m, 2H), 2.68- 2.55 (m, 2H), 1.70-1.65 (m, 1H), 1.44 (t, J = 7.2 Hz, 3H), 0.53-0.39 (m, 4H); LCMS (ESI) m/z: 463.2 [M + H]⁺. | 321 |

### Synthesis of N-((9-ethyl-6-morpholino-8-(pyridin-4-yl)-9H-purin-2-yl)methyl)-3-methylaniline (Compound 322)

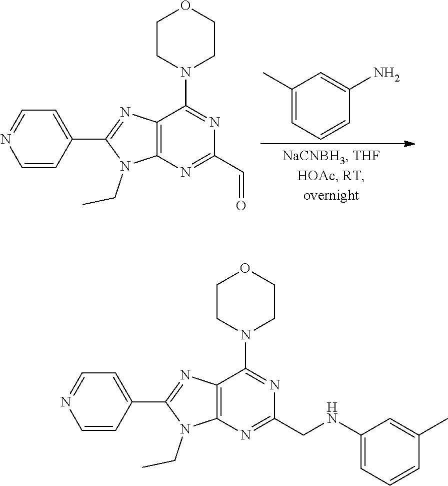

To a solution of 9-ethyl-6-morpholino-8-(pyridin-4-yl)-9H-purine-2-carbaldehyde (80.0 mg, 0.23 mmol) in tetrahydrofuran (5 mL) were added m-toluidine (50.9 mg, 0.47 mmol), sodium cyanoborohydride (20.8 mg, 0.33 mmol) and acetic acid (3 drops). The reaction mixture was stirred at 0° C. for 16 h under N₂ protection and quenched with water. The mixture was extracted with dichloromethane (20 mL*2) and washed with water (10 mL*2). The organic layer was dried over sodium sulfate, filtered and concentrated. The residue was purified by silica gel column chromatography (4% methanol in in dichloromethane). to obtain N-((9-ethyl-6-morpholino-8-(pyridin-4-yl)-9H-purin-2-yl)methyl)-3-methylaniline as white solid (22.4 mg, 22.4%).

¹H NMR (400 MHz, DMSO-d₆) δ 8.81 (d, J=6.7 Hz, 2H), 8.31 (d, J=6.7 Hz, 2H), 6.94 (t, J=7.7 Hz, 1H), 6.58 (s, 1H), 6.50 (d, J=7.4 Hz, 1H), 6.36 (d, J=7.0 Hz, 1H), 5.92 (s, 1H), 4.50-4.05 (m, 8H), 3.73 (s, 4H), 2.17 (s, 3H), 1.36 (t, J=7.1 Hz, 3H); LCMS (ESI) m/z: 430.0 [M+H]⁺.

### Synthesis of 4-(9-ethyl-2-hydrazinyl-8-(pyridin-4-yl)-9H-purin-6-yl)morpholine (Compound 323) and (E)-4-(9-ethyl-2-(2-(3-methylbenzylidene)hydrazinyl)-8-(pyridin-4-yl)-9H-purin-6-yl)morpholine (Compound 324)

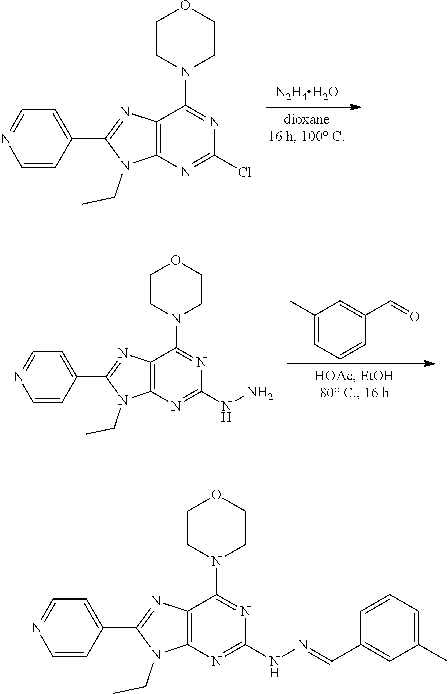

Step 1: Synthesis of 4-(9-ethyl-2-hydrazinyl-8-(pyridin-4-yl)-9H-purin-6-yl)morpholine

A mixture of 4-(2-chloro-9-ethyl-8-(pyridin-4-yl)-9H-purin-6-yl)morpholine (40 mg, 0.116 mmol) hydrazine hydrate (0.5 mL) in dioxane (2 mL) was stirred at 100° C. for 16 h. The reaction mixture was purified by pre-HPLC (BOSTON pHlex ODS 1 0 um 21.2×250 mm 120 A. The mobile phase was acetonitrile/0.1% Ammonium bicarbonate) to give 4-(9-ethyl-2-hydrazinyl-8-(pyridin-4-yl)-9H-purin-6-yl)morpholine as a white solid. (23.7 mg, 60%). ¹H NMR (400 MHz, DMSO-d₆) δ 8.72 (d, J=6.0 Hz, 2H), 7.80-7.72 (m, 2H), 7.63 (s, 1H), 4.65-3.90 (m, 8H), 3.75-3.66 (m, 4H), 1.28 (t, J=7.1 Hz, 3H; LCMS: (ESI) m/z: 341.3 [M+H]+.

### Step 2: Synthesis of (E)-4-(9-ethyl-2-(2-(3-methylbenzylidene)hydrazinyl)-8-(pyridin-4-yl)-9H-purin-6-yl)morpholine

To a solution of 4-(9-ethyl-2-hydrazinyl-8-(pyridin-4-yl)-9H-purin-6-yl)morpholine (82 mg, 0.24 mmol) and 3-methylbenzaldehyde (58 mg, 0.48 mmol) in ethanol (5 mL) was added acetic acid (one drop) and the mixture was stirred at 80° C. for 16 h under argon atmosphere. The mixture was concentrated and the crude product thus obtained was purified by pre-HPLC (BOSTON pHlex ODS 10 μm 21.2×250 mm 120 A. The mobile phase was acetonitrile/0.1% Ammonium bicarbonate) to obtain (E)-4-(9-ethyl-2-(2-(3-methylbenzylidene)hydrazinyl)-8-(pyridin-4-yl)-9H-purin-6-yl)morpholine (52.1 mg, 35%) as a white solid.

1H NMR (400 MHz, DMSO-d₆) δ 10.83 (s, 1H), 8.75 (dd, J=4.5, 1.5 Hz, 2H), 8.08 (s, 1H), 7.79 (dd, J=4.5, 1.6 Hz, 2H), 7.47 (d, J=7.7 Hz, 1H), 7.44 (s, 1H), 7.30 (t, J=7.6 Hz, 1H), 7.15 (d, J=7.5 Hz, 1H), 4.51-4.12 (m, 6H), 3.80-3.70 (m, 4H), 2.35 (s, 3H), 1.31 (t, J=7.2 Hz, 3H); LCMSA011, [M+H]+=442.8.

### Synthesis of (E)-4-(2-(2-(3-methylbenzylidene)hydrazinyl)-8-(pyridin-4-yl)-9H-purin-6-yl)morpholine (Compound 325)

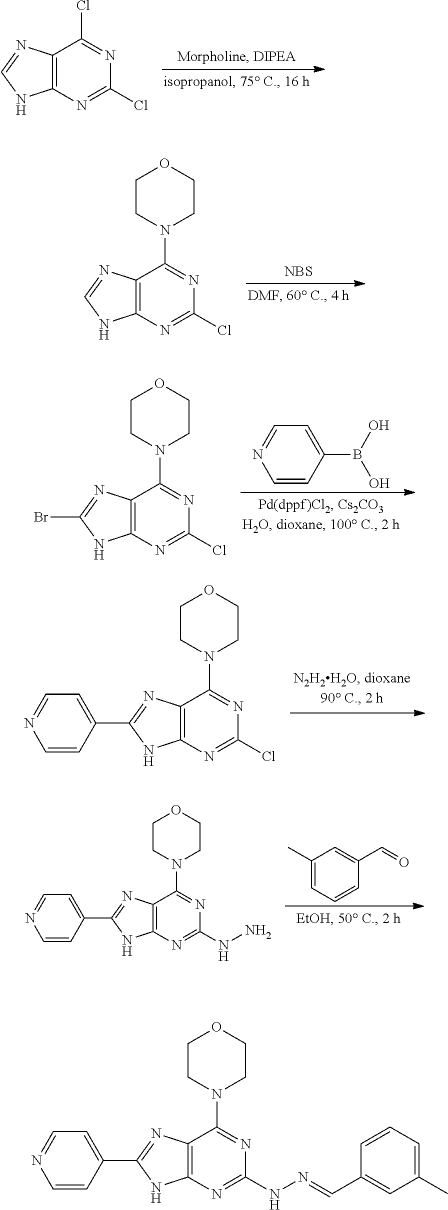

### Step 1: Preparation of 4-(2-chloro-9H-purin-6-yl)morpholine

A solution of 2,6-dichloro-9H-purine (5 g, 26 mmol), morpholine (2 g, 26 mmol) and N,N-diisopropylethylamine (6.7 g, 52 mmol) in isopropanol (200 mL) was stirred at 75° C. for 16 h. The mixture was filtered to obtain 4-(2-chloro-9H-purin-6-yl)morpholine (5 g, 80%) as white solid. ¹H NMR (400 MHz, DMSO-d₆) δ 13.24 (s, 1H), 8.16 (s, 1H), 4.19 (s, 4H), 3.83-3.59 (m, 4H).

### Step 2: Preparation of 4-(8-bromo-2-chloro-9H-purin-6-yl)morpholine

A solution of 4-(2-chloro-9H-purin-6-yl)morpholine (4.8 g, 20 mmol) and N-bromosuccinimide (5.24 g, 30 mmol) in DMF (25 mL) was stirred at 60° C. for 4 h. The mixture was cooled to 20° C. and filtered. The solid was washed with ethyl acetate to obtain 4-(8-bromo-2-chloro-9H-purin-6-yl)morpholine (0.8 g, 13%) as white solid. LCMS (ESI) m/z: 318.0 [M+H]⁺.

### Step 3: Preparation of 4-(2-chloro-8-(pyridin-4-yl)-9H-purin-6-yl)morpholine

A solution of 4-(8-bromo-2-chloro-9H-purin-6-yl)morpholine (0.1 g, 0.31 mmol), pyridin-4-ylboronic acid (0.19 g, 1.57 mmol), [1,1′-Bis(diphenylphosphino)ferrocene]dichloropalladium(II) (0.02 g, 0.03 mmol) and cesium carbonate (0.2 g, 0.62 mmol) in water (0.5 mL) and dioxane (2 mL) was stirred at 100° C. for 2 h under argon. The mixture was diluted with ethyl acetate (10 mL) and washed with water (10 mL). The organic layer was concentrated and purified by Prep-HPLC (Boston C18 21*250 mm 10 μm column. The mobile phase was acetonitrile/0.01% aqueous trifluoroacetic acid.) to give the desired product 4-(2-chloro-8-(pyridin-4-yl)-9H-purin-6-yl)morpholine (0.03 g, 31%) as white solid. LCMS (ESI) m/z: 317.1 [M+H]⁺.

### Step 4: Preparation of 4-(2-hydrazinyl-8-(pyridin-4-yl)-9H-purin-6-yl)morpholine

A solution of 4-(2-chloro-8-(pyridin-4-yl)-9H-purin-6-yl)morpholine (150 mg, 0.47 mmol) and hydrazine hydrate (118 mg, 2.4 mmol) in dioxane (5.0 mL) was stirred at 90° C. under nitrogen for 2 h. The reaction was concentrated and filtered to give the desired product 4-(2-hydrazinyl-8-(pyridin-4-yl)-9H-purin-6-yl)morpholine (100 mg, 68%) as brown solid. LCMS (ESI) m/z: 313.2 [M+H]0.

### Step 5: Preparation of (E)-4-(2-(2-(3-methylbenzylidene)hydrazinyl)-8-(pyridin-4-yl)-9H-purin-6-yl)morpholine

To a solution of 4-(2-hydrazinyl-8-(pyridin-4-yl)-9H-purin-6-yl)morpholine (100 mg, 0.32 mmol) and 3-methylbenzaldehyde (77 mg, 0.64 mmol) in ethanol (5.0 mL) was added acetic acid (19 mg, 0.32 mmol) and the resultant mixture was stirred at 20° C. under nitrogen for 2 h. The mixture was then concentrated and the residue was purified by Prep-HPLC (Boston C18 21*250 mm 10 μm column. The mobile phase was acetonitrile/0.01% aqueous trifluoroacetic acid.) to obtain (E)-4-(2-(2-(3-methylbenzylidene)hydrazinyl)-8-(pyridin-4-yl)-9H-purin-6-yl)morpholine (46.6 mg, 35%) as a yellow solid. 1H NMR (400 MHz, DMSO)) 13.63 (s, 1H), 10.76 (s, 1H), 8.68 (d, J 5.4 Hz, 2H), 8.07 (s, 1H), 7.97 (d, J=5.7 Hz, 2H), 7.56-7.39 (m, 2H), 7.32-7.28 (, 1H), 7.15 (d, J=7.3 Hz, 1H), 4.29-4.24 (m, 4H), 3.78-13.75 (m, 4H), 2.35 (7, 3H); LCMS (ESI) m/z: 415.2 [M+H](.

The following compounds were synthesized according to the protocol described above:

| Name | Structure | NMR, MS | # |
|---|---|---|---|
| (E)-6,6-dimethyl- 2-(2-(3- methylbenzylidene) hydrazinyl)-4- morpholino-8,9- dihydro-6H- [1,4]oxazino[3,4- e]purine | 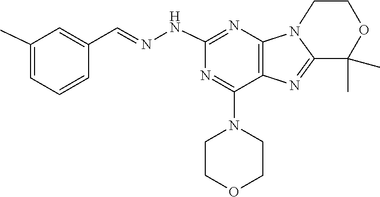 | 1H NMR (400 MHz, DMSO-d₆) δ 10.71 (s, 1H), 8.02 (s, 1H), 7.43 (d, J = 7.4 Hz, 2H), 7.28 (t, J = 7.8 Hz, 1H), 7.13 (d, J = 7.2 Hz, 1H), 4.17 (s, 4H), 4.08 (dd, J = 5.7, 3.8 Hz, 2H), 4.02 (dd, J = 5.3, 3.4 Hz, 2H), 3.80-3.67 (m, 4H), 2.33 (s, 3H), 1.55 (s, 6H); LCMS, [M + H]+ = 422.8. | 326 |
| (E)-4-(2-(2-(3- methoxybenzylidene) hydrazineyl)- 9-methyl-8- (pyridin-4-yl)-9H- purin-6- yl)morpholine | 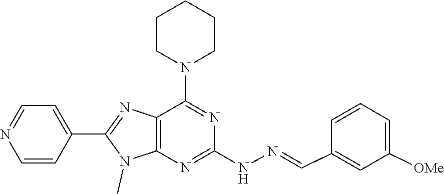 [SMILES: COc1cccc(/C=N/Nc2nc(N3CCCCC3)c3nc(-c4ccncc4)n(C)c3n2)c1] | 1H NMR (400 MHz, DMSO) δ 10.88 (s, 1H), 8.74 (d, J = 6.1 Hz, 2H), 8.07 (s, 1H), 7.86 (m, J = 4.5, 1.6 Hz, 2H), 7.41-7.10 (m, 2H), 6.91 (d, J = 8.0 Hz, 1H), 6.50 (s, 1H), 4.27 (s, 3H), 3.86 (s, 3H), 3.80 (s, 3H), 3.78-3.68 (m, 3H).; LCMS (ESI) m/z: 445.7 [M + H]+. | 327 |
| (E)-4-(9-methyl- 8-(pyridin-4-yl)-2- (2-(1-(m- tolyl)ethylidene) hydrazineyl)-9H- purin-6- yl)morpholine | 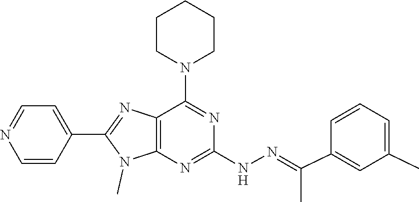 [SMILES: C/C(=N\Nc1nc(N2CCCCC2)c2nc(-c3ccncc3)n(C)c2n1)c1cccc(C)c1] | ¹H NMR (400 MHz, DMSO) δ 9.53 (s, 1H), 8.74 (d, J = 6.1 Hz, 2H), 7.86 (d, J = 6.2 Hz, 2H), 7.64 (d, J = 12.9 Hz, 2H), 7.29 (t, J = 7.8 Hz, 1H), 7.16 (d, J = 7.8 Hz, 1H), 4.29 (s, 4H), 3.87 (s, 3H), 3.76 (s, 4H), 2.36 (s, 3H), 2.29 (s, 3H).; LCMS (ESI) m/z: 443.2 [M + H]+. | 328 |

### Synthesis of (E)-4-(9-methyl-2-(2-(3-methylbenzylidene)hydrazineyl)-8-(pyridin-4-yl)-9H-purin-6-yl)morpholine (Compound 329)

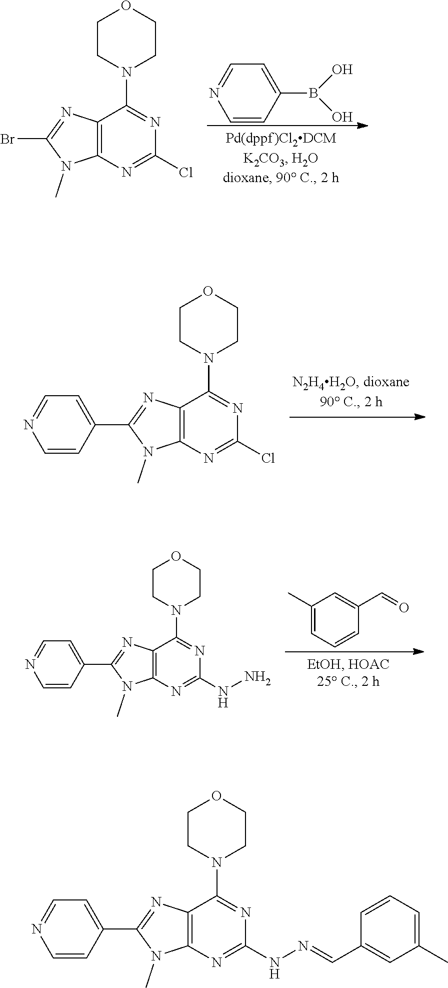

### Step 1: Synthesis of 4-(2-chloro-9-methyl-8-(pyridin-4-yl)-9H-purin-6-yl)morpholine

A mixture of 4-(8-bromo-2-chloro-9-methyl-9H-purin-6-yl)morpholine (4 g, 12 mmol), pyridin-4-ylboronic acid (1.7 g, 14.4 mmol), 1,1′-bis(diphenylphosphino) ferrocene-palladium(II) dichloride (0.45 g, 0.61 mmol) and potassium carbonate (5 g, 36 mmol) in 1,4-dioxane (60 mL) with H₂O (6 mL) was stirred at 90° C. under argon atmosphere for 2 h. The mixture was then concentrated and the residue was purified by silica gel column (petroleum ether:acetic ester=4:1) to afford 4-(2-chloro-9-methyl-8-(pyridin-4-yl)-9H-purin-6-yl)morpholine as white solid. (2.7 g, 66%). LCMS (ESI) m/z: 330.9 [M]+.

### Step 2: Synthesis of 4-(2-hydrazineyl-9-methyl-8-(pyridin-4-yl)-9H-purin-6-yl)morpholine

A mixture of 4-(2-chloro-9-methyl-8-(pyridin-4-yl)-9H-purin-6-yl)morpholine (693 mg, 2.1 mmol) and hydrazine hydrate (2.5 mL) in dioxane (12 mL) was stirred at 100° C. for 2 h. The reaction mixture was concentrated to give 4-(2-hydrazineyl-9-methyl-8-(pyridin-4-yl)-9H-purin-6-yl)morpholine (519.2 mg, 75%). LCMS (ESI) m/z: 327.1 [M+H]+.

### Step 3: Synthesis of (E)-4-(9-methyl-2-(2-(3-methylbenzylidene)hydrazineyl)-8-(pyridin-4-yl)-9H-purin-6-yl)morpholine

A mixture of 4-(2-hydrazineyl-9-methyl-8-(pyridin-4-yl)-9H-purin-6-yl)morpholine (456 mg, 1.4 mmol), 3-methylbenzaldehyde (335 mg, 2.8 mmol) and acetic acid (840 mg, 1.4 mmol) in ethanol (10 mL) was stirred at room temperature under nitrogen atmosphere 2 h. The mixture was filtered and the crude product thus obtained was purified by prep-HPLC (Column Xbridge 21.2*250 mm C18, 10 um, Mobile Phase A: water (10 mmol/L ammonium bicarbonate) B: acetonitrile) to afford 1 (E)-4-(9-methyl-2-(2-(3-methylbenzylidene)hydrazineyl)-8-(pyridin-4-yl)-9H-purin-6-yl)morpholine as yellow solid (387.8 mg, 65%). 1H NMR (400 MHz, DMSO-d₆) δ 10.84 (s, 1H), 8.74 (dd, J=4.5, 1.6 Hz, 2H), 8.07 (s, 1H), 7.87 (dd, J=4.5, 1.6 Hz, 2H), 7.47 (d, J=9.1 Hz, 2H), 7.30 (t, J=7.5 Hz, 1H), 7.15 (d, J=7.5 Hz, 1H), 4.27 (bs, 4H), 3.87 (s, 3H), 3.79-3.73 (m, 4H), 2.35 (s, 3H); LCMS (ESI) m/z: 429.0 [M+H]+.

### Synthesis of (E)-4-(9-ethyl-8-(pyridin-4-yl)-2-(2-(1-(m-tolyl)ethylidene)hydrazineyl)-9H-purin-6-yl)morpholine (Compound 330)

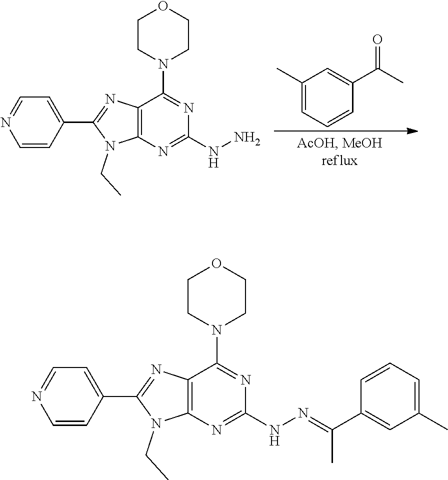

To a solution of 4-(9-ethyl-2-hydrazineyl-8-(pyridin-4-yl)-9H-purin-6-yl)morpholine (100 mg, 0.294 mmol) in methanol (10 mL) were added 1-(m-tolyl)ethan-1-one (48 mg, 0.353 mmol) and acetic acid (3 mL). The mixture was stirred at 80° C. for 8 h and concentrated. The residue was purified by prep-HPLC (0.05% NH4HCO3/H2O:CH3CN=5%-95%) to obtain (E)-4-(9-ethyl-8-(pyridin-4-yl)-2-(2-(1-(m-tolyl)ethylidene)hydrazineyl)-9H-purin-6-yl)morpholine (25.6 mg, 20%) as light yellow solid. ¹H NMR (400 MHz, DMSO-d₆) δ 9.55 (s, 1H), 8.75 (d, J=5.4 Hz, 2H), 7.80 (d, J=5.3 Hz, 2H), 7.71-7.59 (m, 2H), 7.29 (t, J=7.6 Hz, 1H), 7.15 (d, J=7.4 Hz, 1H), 4.34 (q, J=7.2 Hz, 2H), 4.28, (bs, 4H), 3.75 (s, 4H), 2.35, (s,3H), 2.29 (s, 3H), 1.32 (t, J=7.1 Hz, 3H); LCMS (ESI) m/z: 456.8[M+H]+.

### Synthesis of (E)-1-(4-(9-methyl-2-(2-(3-methylbenzylidene)hydrazinyl)-8-(pyridin-4-yl)-9H-purin-6-yl)piperazin-1-yl)ethenone (Compound 331)

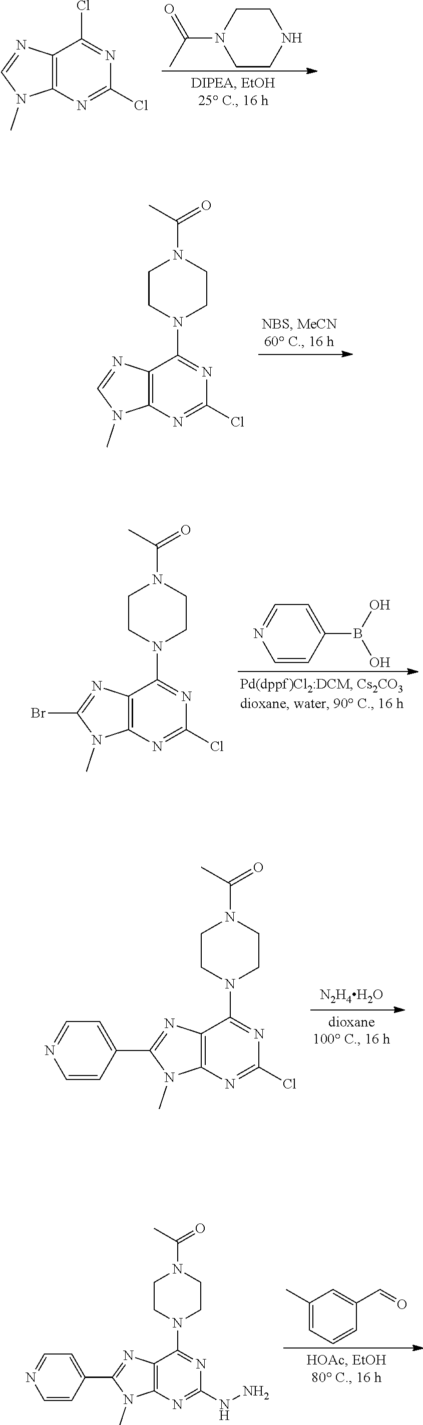

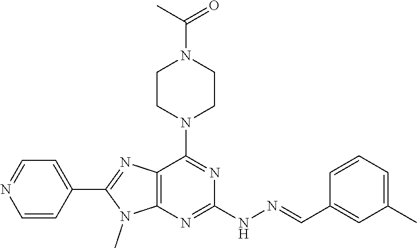 [SMILES: CC(=O)N1CCN(c2nc(N/N=C/c3cccc(C)c3)nc3c2nc(-c2ccncc2)n3C)CC1]

### Step 1: Synthesis of 1-(4-(2-chloro-9-methyl-9H-purin-6-yl)piperazin-1-yl)ethenone

To a solution of 2,6-dichloro-9-methyl-9H-purine (1 g, 4.9 mmol) and 1-(piperazin-1-yl)ethanone (691 mg, 5.4 mmol) in ethanol (20 mL) was added DIPEA (632 mg, 4.9 mmol). The mixture was stirred at 25° C. for 16 h and the resultant precipitate was filtered to give the desired product 1-(4-(2-chloro-9-methyl-9H-purin-6-yl)piperazin-1-yl)ethanone (1.7 g, 83.5%) as white solid. LCMS: [M+H]⁺=295.0.

### Step 2: Synthesis of 1-(4-(8-bromo-2-chloro-9-methyl-9H-purin-6-yl)piperazin-1-yl)ethenone

A solution of 1-(4-(2-chloro-9-methyl-9H-purin-6-yl)piperazin-1-yl)ethanone (1.4 g, 4.75 mmol) and N-bromosuccinimide (1.267 g, 7.12 mmol) in acetonitrile (20 mL) was stirred at 60° C. for 16 h. It was cooled and the resultant precipitate was filtered to give the desired product (1.4 g, 65%) as white solid. LCMS: [M+H]⁺=373.0.

### Step 3: Synthesis of 1-(4-(2-chloro-9-methyl-8-(pyridin-4-yl)-9H-purin-6-yl)piperazin-1-yl)ethenone

A mixture of 1-(4-(8-bromo-2-chloro-9-methyl-9H-purin-6-yl)piperazin-1-yl)ethanone (373 mg, 1 mmol), pyridin-4-ylboronic acid (123 mg, 1 mmol), 1,1′-bis(diphenylphosphino) ferrocene-palladium(II) dichloride dichloromethane complex (82 mg, 0.1 mmol) and cesium carbonate (978 mg, 3 mmol) in water (1 mL) and dioxane (10 mL) was stirred at 90° C. for 16 h under argon. The reaction mixture was concentrated and the crude product was purified by flash chromatography (Biotage, 80 g silica gel, methanol/dichloromethane=5%-10%) to obtain 1-(4-(2-chloro-9-methyl-8-(pyridin-4-yl)-9H-purin-6-yl)piperazin-1-yl)ethanone (260 mg, 63.5%) as white solid. LCMS: MS (ESI) m/z 372.1 [M+H]⁺.

### Step 4: Synthesis of 1-(4-(2-hydrazinyl-9-methyl-8-(pyridin-4-yl)-9H-purin-6-yl)piperazin-1-yl)ethenone

A mixture of 1-(4-(2-chloro-9-methyl-8-(pyridin-4-yl)-9H-purin-6-yl)piperazin-1-yl)ethanone (223 mg, 0.6 mmol) and hydrazine hydrate (2 mL) in dioxane (6 mL) was stirred at 100° C. for 16 h. The reaction mixture was concentrated to give 1-(4-(2-hydrazinyl-9-methyl-8-(pyridin-4-yl)-9H-purin-6-yl)piperazin-1-yl)ethanone as white solid. (290 mg, crude). It was directly used in the next step without purification. LCMS: (ESI) m/z: 368.1 [M+H]+.

### Step 5: Synthesis of (E)-1-(4-(9-methyl-2-(2-(3-methylbenzylidene)hydrazinyl)-8-(pyridin-4-yl)-9H-purin-6-yl)piperazin-1-yl)ethenone

To a solution of 1-(4-(2-hydrazinyl-9-methyl-8-(pyridin-4-yl)-9H-purin-6-yl)piperazin-1-yl)ethanone (250 mg, 0.6 mmol) and 3-methylbenzaldehyde (144 mg, 1.2 mmol) in ethanol (20 mL) was added acetic acid (two dros) and the mixture was stirred at 80° C. for 16 h under argon. The mixture was concentrated and the residue was purified by pre-HPLC (BOSTON pHlex ODS 10 μm 21.2×250 mm 120 A. The mobile phase was acetonitrile/0.1% Ammonium bicarbonate) to obtain (E)-1-(4-(9-methyl-2-(2-(3-methylbenzylidene)hydrazinyl)-8-(pyridin-4-yl)-9H-purin-6-yl)piperazin-1-yl)ethanone (80.3 mg, 24.4%) as a white solid.

1H NMR (400 MHz, DMSO-d₆) δ 10.88 (s, 1H), 8.75 (dd, J=4.5, 1.5 Hz, 2H), 8.08 (s, 1H), 7.88 (dd, J=4.6, 1.5 Hz, 2H), 7.48 (d, J=8.9 Hz, 2H), 7.31 (t, J=7.5 Hz, 1H), 7.16 (d, J=7.5 Hz, 1H), 4.45-4.01 (m, 4H), 3.87 (s, 3H), 3.62 (s, 4H), 2.35 (s, 3H), 2.08 (s, 3H); LCMS: [M+H]⁺=470.0.

### Synthesis of (E)-4-(9-ethyl-2-(1-methyl-2-(3-methylbenzylidene)hydrazineyl)-8-(pyridin-4-yl)-9H-purin-6-yl)morpholine (Compound 332)

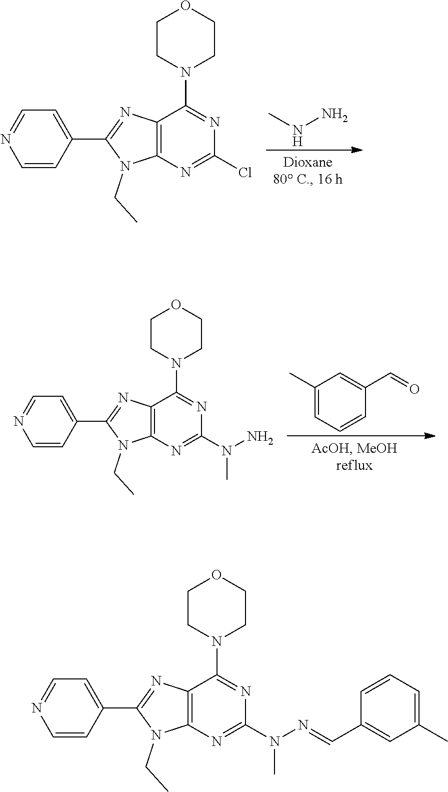

### Step 1: Preparation of 4-(9-ethyl-2-(1-methylhydrazineyl)-8-(pyridin-4-yl)-9H-purin-6-yl)morpholine

To a solution of 4-(2-chloro-9-ethyl-8-(pyridin-4-yl)-9H-purin-6-yl)morpholine (150 mg, 1.0 eq) in dioxane (10 mL) was added methylhydrazine (5 mL). The mixture was stirred at 80° C. for 3 h. Then the reaction was diluted with water (5 mL) and extracted with EtOAc (20*3 mL). The organic layer was combined, washed with brine (30 mL), dried over Na2SO4, filtered and concentrated. The residue was purified by flash chromatography on silica gel (petroleum ether:ethyl acetate=75:25) to give product as yellow solid. (160 mg, 100%).

### Step 2: Preparation of (E)-4-(9-ethyl-2-(1-methyl-2-(3-methylbenzylidene)hydrazineyl)-8-(pyridin-4-yl)-9H-purin-6-yl)morpholine

To a solution of 4-(9-ethyl-2-(1-methylhydrazineyl)-8-(pyridin-4-yl)-9H-purin-6-yl)morpholine (160 mg, 0.452 mmol) in methanol (10 mL) were added 3-methylbenzaldehyde (65 mg, 0.542 mmol) and acetic acid (3 mL). The mixture was stirred at 80° C. for 8 h and concentrated. The residue was purified by Prep-HPLC (0.05% NH4HCO3/H2O:CH3CN=5%-95%) to offer (E)-4-(9-ethyl-8-(pyridin-4-yl)-2-(2-(1-(m-tolyl)ethylidene)hydrazineyl)-9H-purin-6-yl)morpholine (25.8 mg, 12%) as a light yellow solid.

1H NMR (400 MHz, DMSO-d₆) δ 8.76 (dd, J=4.5, 1.6 Hz, 2H), 7.90-7.76 (m, 3H), 7.69-7.54 (m, 2H), 7.31 (t, J=7.6 Hz, 1H), 7.14 (d, J=7.6 Hz, 1H), 4.55-4.14 (m, 6H), 3.86-3.74 (m, 4H), 3.69 (s, 3H), 2.35 (s, 3H), 1.36 (t, J=7.2 Hz, 3H); LCMS (ESI) m/z: 456.9[M]+.

### Synthesis of 3-methyl-N′-(9-methyl-6-morpholino-8-(pyridin-4-yl)-9H-purin-2-yl)benzohydrazide (Compound 333)

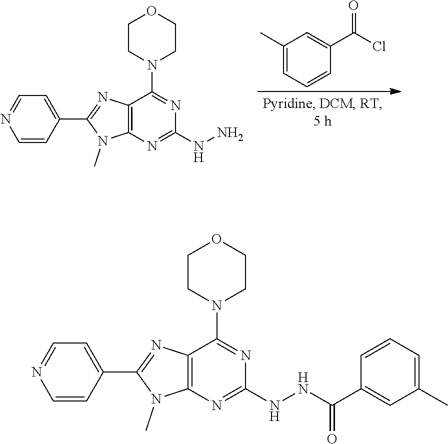

To a solution of 4-(2-hydrazineyl-9-methyl-8-(pyridin-4-yl)-9H-purin-6-yl)morpholine (120 mg, 0.368 mmol) in dichloromethane (10 mL) were added 3-methylbenzoyl chloride (556 mg, 3.68 mmol) and pyridide (59 mg, 0.736 mmol). The mixture was stirred at 30° C. for 5 h and concentrated. The residue was purified by Prep-HPLC (0.05% NH4HCO3/H2O:CH3CN=5%-95%) to obtain 3-methyl-N′-(9-methyl-6-morpholino-8-(pyridin-4-yl)-9H-purin-2-yl)benzohydrazide (15.4 mg, 9.4%) as a white solid.

¹H NMR (400 MHz, DMSO-d₆) δ 8.72 (dd, J=4.6, 1.5 Hz, 2H), 8.53 (s, 1H), 7.83 (dd, J=4.6, 1.6 Hz, 2H), 7.76-7.68 (m, 2H), 7.39 (dd, J=8.7, 4.5 Hz, 2H), 4.19 (s, 4H), 3.75 (s, 3H), 3.69 (s, 4H), 2.38 (s, 3H). LCMS (ESI) m/z: 445.1[M+H]+.

### Synthesis of (E)-4-(9-ethyl-8-(pyridin-4-yl)-2-((2-(m-tolyl)hydrazineylidene)methyl)-9H-purin-6-yl)morpholine (Compound 334)

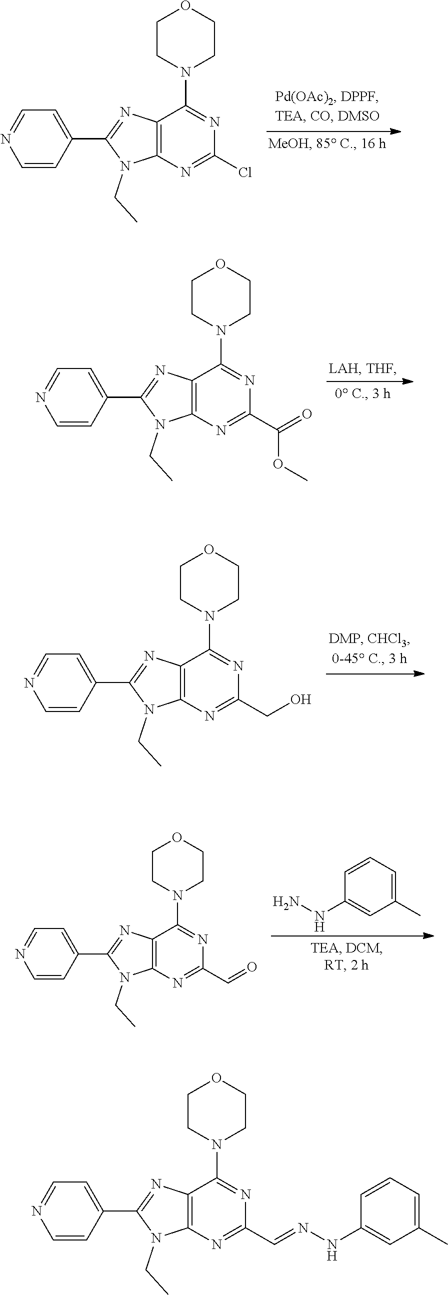

### Step 1: Preparation of methyl methyl 9-ethyl-6-morpholino-8-(pyridin-4-yl)-9H-purine-2-carboxylate

To a solution of 4-(2-chloro-9-ethyl-8-(pyridin-4-yl)-9H-purin-6-yl)morpholine (1.0 g, 2.9 mmol) in dimethyl sulfoxide (40 mL) and methanol (50 mL) were added palladium (II) acetate (64.9 mg, 0.29 mmol), 1,1′-bis(diphenylphosphino)ferrocene (321.3 mg, 5.8 mmol) and triethylamine (878.7 mg, 8.7 mmol) and the reaction was stirred at 80° C. for 16 h under carbon monoxide atmosphere. The mixture was extracted with dichloromethane (20 mL*2) and washed with water (10 mL*2). The organic layer was dried over sodium sulfate, and concentrated. The residue was purified by silica gel column chromatography (3% methanol in dichloromethane). The mixture was concentrated to give the product methyl 9-ethyl-6-morpholino-8-(pyridin-4-yl)-9H-purine-2-carboxylate as white solid (1.0 g, 93.7%). LCMS (ESI) m/z: 369.0 [M+H]⁺.

### Step 2: Preparation of (9-ethyl-6-morpholino-8-(pyridin-4-yl)-9H-purin-2-yl)methanol

To a solution of methyl 9-ethyl-6-morpholino-8-(pyridin-4-yl)-9H-purine-2-carboxylate (1.0 mg, 2.71 mmol) in dichloromethane (20 mL) was added lithium aluminum hydride (319.2 mgmg, 8.15 mmol) under nitrogen protection, and the reaction was stirred at 0° C. for 0.5 h, then warmed to room temperature and stirred for 3 h at 25° C. The mixture was extracted with dichloromethane (20 mL*2) and washed with water (10 mL*2). The organic layer was dried over sodium sulfate and concentrated. The residue was purified by silica gel column chromatography (3% methanol in dichloromethane, the mobile phase was acetonitrile/0.1% Ammonium bicarbonate) to give the product (9-ethyl-6-morpholino-8-(pyridin-4-yl)-9H-purin-2-yl)methanol as white solid (1.0 g, 97.7%). LCMS (ESI) m/z: 341.0 [M+H]⁺.

### Step 3: Preparation of 9-ethyl-6-morpholino-8-(pyridin-4-yl)-9H-purine-2-carbaldehyde

To a solution of (9-ethyl-6-morpholino-8-(pyridin-4-yl)-9H-purin-2-yl)methanol (1.0 mg, 2.94 mmol) in chloroform (50 mL) was added Des-Martin periodinane (1.87 g, 8.82 mmol) under N₂ protection and the reaction was stirred at 0° C. for 0.5 h. Then, the solution was stirred for 3 h at 45° C. The mixture was extracted with dichloromethane (20 mL*2) and washed with water (10 mL*2). The organic layer was dried over sodium sulfate and concentrated. The residue was purified by silica gel column chromatography (5% methanol in dichloromethane, the mobile phase was acetonitrile/0.1% Ammonium bicarbonate) to give the product 9-ethyl-6-morpholino-8-(pyridin-4-yl)-9H-purine-2-carbaldehyde as white solid (500 mg, 50.3%). LCMS (ESI) m/z: 339.1 [M+H]⁺.

### Step 4: Preparation of (E)-4-(9-ethyl-8-(pyridin-4-yl)-2-((2-(m-tolyl)hydrazineylidene)methyl)-9H-purin-6-yl)morpholine

To a solution of 9-ethyl-6-morpholino-8-(pyridin-4-yl)-9H-purine-2-carbaldehyde (300.0 mg, 0.88 mmol) in dichloromethane (10 mL) was added m-tolylhydrazine (162.5 mg, 1.33 mmol) and triethylamine (273.2 mg, 2.66 mmol) under nitrogen protection, the reaction was stirred at 25° C. for 2 h. The mixture was extracted with dichloromethane (20 mL*2) and washed with water (10 mL*2). The organic layer was dried over sodium sulfate and concentrated. The crude product was purified by pre-HPLC (BOSTON pHlex ODS 10 μm 21.2×250 mm 120 A) to obtain (E)-4-(9-ethyl-8-(pyridin-4-yl)-2-((2-(m-tolyl)hydrazineylidene)methyl)-9H-purin-6-yl)morpholine as white solid (196.0 mg, 50.3%).

¹H NMR (400 MHz, DMSO-d₆) δ 10.66 (s, 1H), 8.79 (dd, J=4.5, 1.6 Hz, 2H), 7.85 (dd, J=4.5, 1.6 Hz, 2H), 7.80 (s, 1H), 7.14 (t, J=7.7 Hz, 1H), 7.01-6.86 (m, 2H), 6.64 (d, J=7.3 Hz, 1H), 4.41 (q, J=7.2 Hz, 2H), 4.30 (bs, 4H), 3.90-3.64 (m, 4H), 2.28 (s, 3H), 1.33 (t, J=7.2 Hz, 3H); LCMS (ESI) m/z: 443.1 [M+H]⁺.

### Synthesis of (E)-4-(9-ethyl-2-((2-methyl-2-(m-tolyl)hydrazineylidene)methyl)-8-(pyridin-4-yl)-9H-purin-6-yl)morpholine (Compound 335)

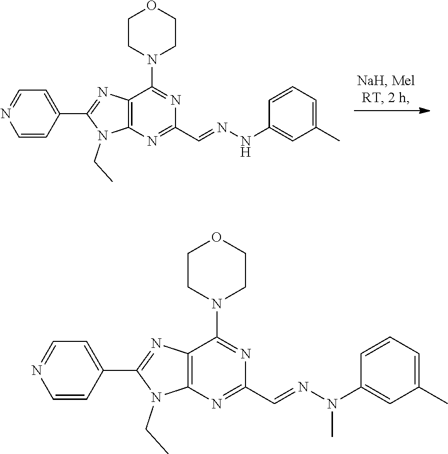 [SMILES: CCn1c(-c2ccncc2)nc2c(N3CCOCC3)nc(/C=N/Nc3cccc(C)c3)nc21 ; CCn1c(-c2ccncc2)nc2c(N3CCOCC3)nc(/C=N/N(C)c3cccc(C)c3)nc21]

To a solution of (E)-4-(9-ethyl-8-(pyridin-4-yl)-2-((2-(m-tolyl)hydrazineylidene)methyl)-9H-purin-6-yl)morpholine (150.0 mg, 0.88 mmol) and sodium hydride (16.3 mg, 2.66 mmol) in tetrahydrofuran (10 mL) was added Iodomethane (48.2 mg, 2.66 mmol) under ice-bath. The mixture was stirred at 0˜25° C. for 2.0 h, diluted with water and extracted with ethyl acetate (20 mL*2). The combined organic layer was washed with water (10 mL*2), dried over sodium sulfate, and concentrated. The crude product was purified by pre-HPLC (BOSTON pHlex ODS 10 μm 21.2×250 mm 120 A) to obtain (E)-4-(9-ethyl-2-((2-methyl-2-(m-tolyl)hydrazineylidene)methyl)-8-(pyridin-4-yl)-9H-purin-6-yl)morpholine as white solid (42.5 mg, 27.4%).

¹H NMR (500 MHz, DMSO-d₆) δ 8.79 (d, J=6.0 Hz, 2H), 7.85 (dd, J=4.6, 1.5 Hz, 2H), 7.51 (s, 1H), 7.34 (s, 1H), 7.28 (d, J=8.3 Hz, 1H), 7.21 (t, J=7.8 Hz, 1H), 6.77 (d, J=7.3 Hz, 1H), 4.41 (q, J=7.2 Hz, 2H), 4.39 (bs, 4H), 3.82-3.72 (m, 4H), 3.46 (s, 3H), 2.33 (s, 3H), 1.35 (t, J=7.2 Hz, 3H); LCMS (ESI) m/z: 457.1 [M+H]⁺.

### Synthesis of (E)-4-(9-ethyl-8-(pyridin-4-yl)-2-(1-(2-m-tolylhydrazono)ethyl)-9H-purin-6-yl)morpholine (Compound 336)

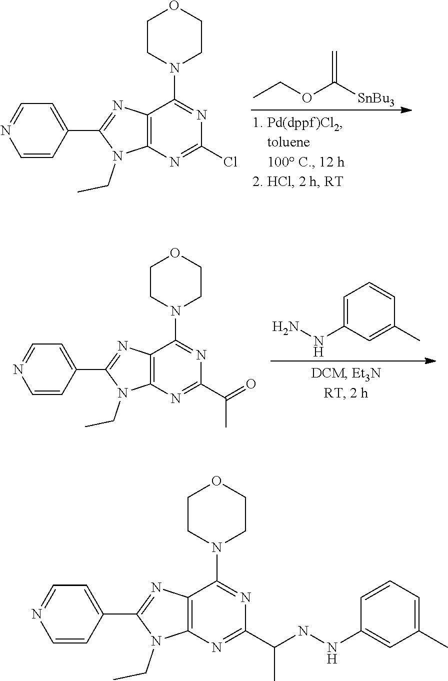

### Step 1: Preparation of 1-(9-ethyl-6-morpholino-8-(pyridin-4-yl)-9H-purin-2-yl)ethenone

To a solution of 4-(2-chloro-9-ethyl-8-(pyridin-4-yl)-9H-purin-6-yl)morpholine (100 mg, 0.29 mmol) in toluene (5 mL) was added tributyl(1-ethoxyvinyl)stannane (263.4 mg, 0.73 mmol) and 1,1′-bis(diphenylphosphino)ferrocene-palladium(II)dichloride (11 mg, 0.015 mmol) at 25° C. The resultant mixture was stirred at 100° C. for 12 h under nitrogen protection. Then hydrochloric acid (5 mL) was added under N₂ protection and the mixture was stirred at 25° C. for 2 h. The mixture was extracted with ethyl acetate (20 mL*2) and washed with water (10 mL*2). The organic layer was dried over sodium sulphate, filtered and concentrated under the reduced pressure. The residual was purified by silica gel column chromatography (petroleum/ethyl acetate=1/1) to obtain 1-(9-ethyl-6-morpholino-8-(pyridin-4-yl)-9H-purin-2-yl)ethanone (70 mg, 70%) as yellow oil. LCMS (ESI) m/z: 352.9 [M+H]⁺.

### Step 2: Preparation of (E)-4-(9-ethyl-8-(pyridin-4-yl)-2-(1-(2-m-tolylhydrazono)ethyl)-9H-purin-6-yl)morpholine

A solution of 1-(9-ethyl-6-morpholino-8-(pyridin-4-yl)-9H-purin-2-yl)ethanone (110 mg, 0.31 mmol), m-tolylhydrazine (57 mg, 0.47 mmol) and triethylamine (63 mg, 0.62 mmol) in dichloromethane (10 mL) was stirred at 20° C. for 2 h under argon. The mixture was diluted with dichloromethane (25 mL) and washed with water (25 mL). The organic layer was dried on sodium sulphate, filtered and concentrated under the reduced pressure. And the residue was purified by HPLC ((BOSTON pHlex ODS 10 μm 21.2×250 mm 120 A. The mobile phase was acetonitrile/0.1% Ammonium bicarbonate) to give (E)-4-(9-ethyl-8-(pyridin-4-yl)-2-(1-(2-m-tolylhydrazono)ethyl)-9H-purin-6-yl)morpholine (22.8 mg, 16%)) as white solid.

¹H NMR (400 MHz, DMSO-d₆) δ 9.44 (s, 1H), 8.79 (d, J=6.0 Hz, 2H), 7.85 (dd, J=4.5, 1.6 Hz, 2H), 7.34-7.01 (m, 3H), 6.62 (d, J=5.5 Hz, 1H), 4.42 (q, J=7.2 Hz, 2H), 4.43-4.13 (m, 4H), 3.78 (s, 4H), 2.36 (s, 3H), 2.30 (s, 3H), 1.36 (t, J=7.2 Hz, 3H)I LCMS (ESI) m/z: 457.2 [M+H]⁺.

### Synthesis of (E)-9-ethyl-6-morpholino-8-(pyridin-4-yl)-9H-purine-2-carbaldehyde O-(m-tolyl) oxime (Compound 337)

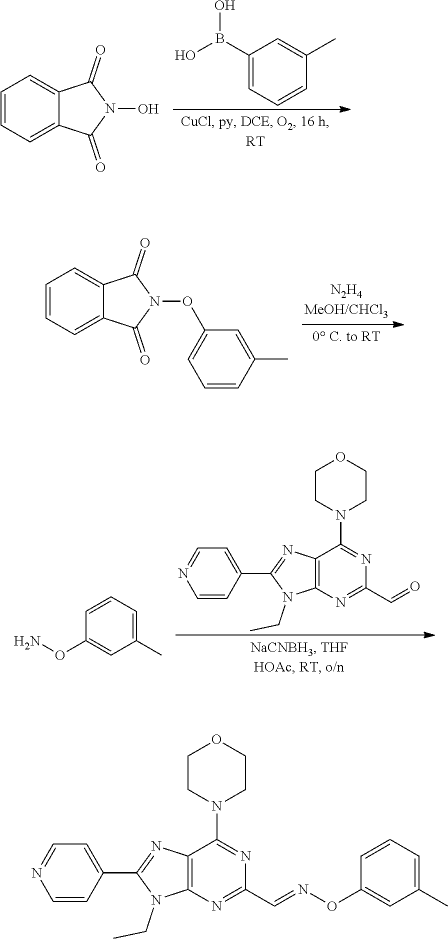

### Step 1: Preparation of 2-(m-tolyloxy)isoindoline-1,3-dione

To a solution of 2-hydroxyisoindoline-1,3-dione (500.0 mg, 3.06 mmol) in 1-2-dichoroethane (20 mL) was added m-tolylboronic acid (833.4 mg, 6.13 mmol), copper chloride (303.4 mg, 3.06 mmol), pyridine (266.7 mg, 3.37 mmol) and 4-A molecular sieves (0.8 g) at 0° C., the reaction was stirred at 25° C. for 16 h under oxygen protection. The mixture was diluted with water (10 mL*2) and extracted with ethyl acetate (20 mL*2). The organic layer was dried over sodium sulfate, filtered and concentrated. The residue was purified by silica gel column chromatography (17% ethyl acetate in petroleum ether) to give the product 2-(m-tolyloxy)isoindoline-1,3-dione as colorless oil (350.0 mg, 45.1%). LCMS (ESI) m/z: 254.1 [M+H]⁺.

### Step 2: Preparation of O-(m-tolyl)hydroxylamine

To a solution of 2-(m-tolyloxy)isoindoline-1,3-dione (300.0 mg, 1.18 mmol) in methanol/chloroform (30 mL) was added hydrazine (178.0 mg, 3.56 mmol) at 0° C., the reaction was stirred at 25° C. for 2 h under nitrogen protection. The mixture was then extracted with ethyl acetate (20 mL*2) and washed with water (10 mL*2). The organic layer was dried over sodium sulfate, filtered and concentrated. The residue was purified by silica gel column chromatography (12% ethyl acetate petroleum ether) to give the product 0-(m-tolyl)hydroxylamine as colorless oil (110.0 mg, 64.6%). LCMS (ESI) m/z: 124.3 [M+H]⁺.

### Step 3: Preparation of (E)-9-ethyl-6-morpholino-8-(pyridin-4-yl)-9H-purine-2-carbaldehyde O-(m-tolyl) oxime

To a solution of 9-ethyl-6-morpholino-8-(pyridin-4-yl)-9H-purine-2-carbaldehyde (302.2 mg, 0.89 mmol) in tetrahydrofuran (20 mL) were added O-(m-tolyl)hydroxylamine (110.0 mg, 0.89 mmol) and acetic acid (5.3 mg, 0.08 mmol) at 25° C. and the reaction mixture was stirred for 2 h under nitrogen protection. The mixture was diluted with water (10 mL) and extracted with ethyl acetate (20 mL*2). The organic layer was dried over sodium sulfate, filtered and concentrated. The residue was purified by silica gel column chromatography (4% methanol in dichloromethane) to give the product (E)-9-ethyl-6-morpholino-8-(pyridin-4-yl)-9H-purine-2-carbaldehyde O-(m-tolyl) oxime as white solid (88.9 mg, 22.7%). ¹H NMR (400 MHz, DMSO-d₆) δ 8.81 (dd, J=4.5, 1.5 Hz, 2H), 8.48 (s, 1H), 7.86 (dd, J=4.5, 1.6 Hz, 2H), 7.28 (t, J=8.1 Hz, 1H), 7.08 (d, J=6.8 Hz, 2H), 6.92 (d, J=7.3 Hz, 1H), 4.43 (q, J=7.2 Hz, 2H), 4.25 (bs, 4H), 3.84-3.71 (m, 4H), 2.34 (s, 3H), 1.33 (t, J=7.2 Hz, 3H); LCMS (ESI) m/z: 444.1 [M+H]⁺.

### Synthesis of (E)-1-(9-ethyl-6-morpholino-8-(pyridin-4-yl)-9H-purin-2-yl)ethan-1-one O-benzyl oxime (Compound 338)

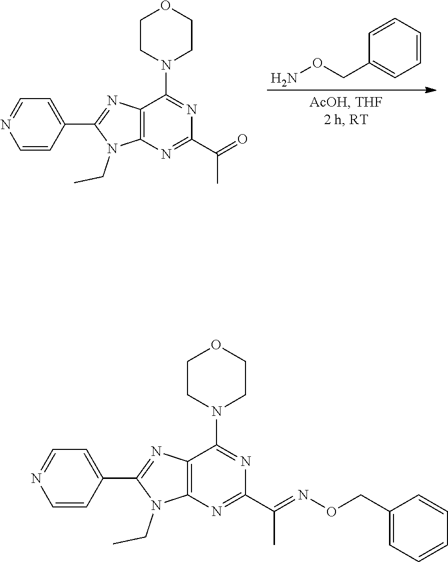 [SMILES: CCn1c(-c2ccncc2)nc2c(N3CCOCC3)nc(C(C)=O)nc21 ; NOCc1ccccc1 ; CCn1c(-c2ccncc2)nc2c(N3CCOCC3)nc(/C(C)=N/OCc3ccccc3)nc21]

To a solution of 1-(9-ethyl-6-morpholino-8-(pyridin-4-yl)-9H-purin-2-yl)ethan-1-one (150 mg, 0.43 mmol) in tetrahydrofuran (10 mL) was added O-benzylhydroxylamine (57.7 mg, 0.47 mmol), acetic acid (2.6 mg, 0.04 mmol) at 25° C. and the reaction mixture was stirred at 25° C. for 2 h under nitrogen atmosphere. The mixture was extracted with ethyl acetate (20 mL*2), washed with water (10 mL*2), dried over sodium sulfate, and concentrated. The residue was purified by silica gel column chromatography (4% methanol in dichloromethane) to obtain (E)-1-(9-ethyl-6-morpholino-8-(pyridin-4-yl)-9H-purin-2-yl)ethan-1-one O-benzyl oxime (92.7 mg, 46.6%) as white solid.

¹H NMR (400 MHz, DMSO-d₆) δ 8.80 (d, J=5.9 Hz, 2H), 7.84 (d, J=6.0 Hz, 2H), 7.45 (d, J=7.1 Hz, 2H), 7.39 (t, J=7.2 Hz, 2H), 7.34 (d, J=7.1 Hz, 1H), 5.26 (s, 2H), 4.39 (q, J=7.2 Hz, 2H), 4.25 (bs, 4H), 3.81-3.69 (m, 4H), 2.31 (s, 3H), 1.31 (t, J=7.2 Hz, 3H); LCMS (ESI) m/z: 458.4 [M+H]⁺.

### Synthesis of 1-(9-ethyl-2-(3-(1-methyl-1H-pyrazol-3-yl)phenyl)-6-(piperidin-1-yl)-9H-purin-8-yl)ethan-1-ol (COMPOUND 339)

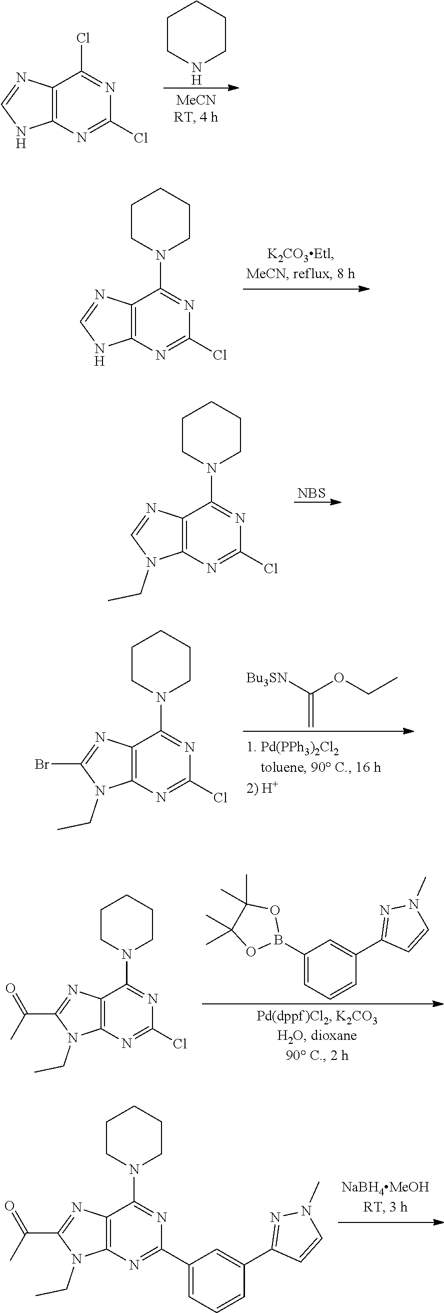

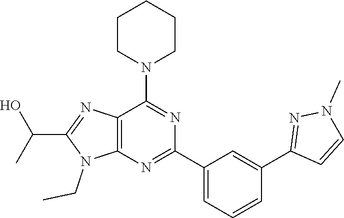 [SMILES: CCn1c(C(C)O)nc2c(N3CCCCC3)nc(-c3cccc(-c4ccn(C)n4)c3)nc21]

### Step 1: Synthesis of 2-chloro-6-(piperidin-1-yl)-9H-purine

To a solution of 2,6-dichloro-9H-purine (2.5 g, 13.2 mmol) in acetonitrile (100 mL) was added piperidine (2.2 g, 26.4 mmol) and the resulting mixture was stirred at 30° C. for 4 h. Then the reaction was quenched with water (100 mL) and the mixture was extracted with ethyl acetate (50 mL*3). The combined organic layer was washed with brine (100 mL), dried over sodium sulfate, filtered and concentrated. The resultant residue was subjected to flash chromatography on silica gel (petroleum ether:ethyl acetate=75:25) to obtain 2-chloro-6-(piperidin-1-yl)-9H-purine (3.0 g, 95.5%) as yellow solid. LCMS (ESI) m/z: 237.9[M]⁺.

### Step 2: Synthesis of 2-chloro-9-ethyl-6-(piperidin-1-yl)-9H-purine

To a solution of 2-chloro-6-(piperidin-1-yl)-9H-purine (3.0 g, 12.7 mmol) in acetonitrile (100 mL) were added iodoethane (3.9 g, 25.3 mmol) and potassium carbonate (3.5 g, 25.3 mmol) and the resulting mixture was stirred at 90° C. for 8 h. Then the reaction was quenched with water (150 mL) and extracted with ethyl acetate (50 mL*3). The organic layers were combined, washed with brine (30 mL), dried over sodium sulfate, filtered and concentrated. The resulting residue was subjected to flash chromatography (petroleum ether:ethyl acetate=75:25) to obtain 2-chloro-9-ethyl-6-(piperidin-1-yl)-9H-purine (2.7 g, 80.9%) as yellow solid. LCMS (ESI) m/z: 266.1 [M+H]⁺.

### Step 3: Synthesis of 8-bromo-2-chloro-9-ethyl-6-(piperidin-1-yl)-9H-purine

To a solution of 2-chloro-9-ethyl-6-(piperidin-1-yl)-9H-purine (500 mg, 1.89 mmol) in acetonitrile (10 mL) was added 1-bromopyrrolidine-2,5-dione (403 mg, 2.26 mmol) and the resulting mixture was stirred at 25° C. for 4 h. The reaction was then quenched with water (25 mL) and the mixture was extracted with ethyl acetate (20 mL*3). The organic layer was combined, washed with brine (30 mL), dried over sodium sulfate, filtered and concentrated. The resultant crude product was purified by flash chromatography on silica gel (petroleum ether:ethyl acetate=75:25) to obtain 8-bromo-2-chloro-9-ethyl-6-(piperidin-1-yl)-9H-purine (375 mg, 57.7%) as white solid. LCMS (ESI) m/z: 343.6[M+H]⁺.

### Step 4: Synthesis of 1-(2-chloro-9-ethyl-6-(piperidin-1-yl)-9H-purin-8-yl)ethan-1-one

To a solution of 8-bromo-2-chloro-9-ethyl-6-(piperidin-1-yl)-9H-purine (170 mg, 0.49 mmol) in toluene (10 mL) were added tributyl(1-ethoxyvinyl)stannane (356 mg, 0.99 mmol) and dichloro[bis(triphenylphosphine)]palladium(II) (50 mg, 0.071 mmol). The resultant reaction mixture was stirred at 90° C. for 16 h under nitrogen followed by the addition of HCl (3N, 6 mL, aqueous) and the mixture was stirred further for 1 h. The reaction was then quenched with water (15 mL) and the mixture was extracted with ethyl acetate (20 mL*3). The organic layers were combined, washed with brine (30 mL), dried over sodium sulfate, filtered and concentrated. The residue was subjected to prep-HPLC (0.05% ammonium bicarbonate:acetonitrile=5%˜95%)) to obtain 1-(2-chloro-9-ethyl-6-(piperidin-1-yl)-9H-purin-8-yl)ethan-1-one (110 mg, 72.3%) as white solid. LCMS (ESI) m/z: 308.1[M+H]⁺.

### Step 5: Synthesis of 1-(9-ethyl-2-(3-(1-methyl-1H-pyrazol-3-yl)phenyl)-6-(piperidin-1-yl)-9H-purin-8-yl)ethan-1-one

To a solution of 1-(2-chloro-9-ethyl-6-(piperidin-1-yl)-9H-purin-8-yl)ethan-1-one (110 mg, 0.36 mmol) in dioxane (5 mL) and water (1 mL) were added 1-methyl-3-(3-(4,4,5,5-tetramethyl-1,3,2-dioxaborolan-2-yl)phenyl)-1H-pyrazole (255 mg, 0.90 mmol), potassium carbonate (100 mg, 0.72 mmol) and dichloro[1,1′-bis(diphenylphosphino)ferrocene]palladium(II) dichloromethane (29 mg, 0.036 mmol). The resultant mixture was stirred at 90° C. for 2 h, then quenched with water (15 mL) and extracted with ethyl acetate (20 mL*3). The organic layers were combined, washed with brine (30 mL), dried over sodium sulfate, filtered and concentrated. The residue was subjected to flash chromatography on silica gel (petroleum ether:ethyl acetate=75:25) to obtain 1-(9-ethyl-2-(3-(1-methyl-1H-pyrazol-3-yl)phenyl)-6-(piperidin-1-yl)-9H-purin-8-yl)ethan-1-one (100 mg, 65.6%) as yellow solid. LCMS (ESI) m/z: 430.1 [M+H]⁺.

### Step 6: Synthesis of 1-(9-ethyl-2-(3-(1-methyl-1H-pyrazol-3-yl)phenyl)-6-(piperidin-1-yl)-9H-purin-8-yl)ethan-1-ol

To a solution of 1-(9-ethyl-2-(3-(1-methyl-1H-pyrazol-3-yl)phenyl)-6-(piperidin-1-yl)-9H-purin-8-yl)ethan-1-one (65 mg, 0.15 mmol) in methanol (10 mL) was added sodium borohydride (11 mg, 0.30 mmol). The mixture reaction was stirred at 30° C. for 4 h, then quenched with water (15 mL) and extracted with ethyl acetate (20 mL*3). The organic layers were combined, washed with brine (30 mL), dried over sodium sulfate, filtered and concentrated. The residue was subjected to prep-HPLC HPLC (0.05% ammonium bicarbonate:acetonitrile=5%˜95%) to obtain 1-(9-ethyl-2-(3-(1-methyl-1H-pyrazol-3-yl)phenyl)-6-(piperidin-1-yl)-9H-purin-8-yl)ethan-1-ol (42.3 mg, 64.5%) as white solid. ¹H NMR (400 MHz, DMSO-d₆) δ 8.75 (t, J=1.5 Hz, 1H), 8.35-8.25 (m, 1H), 7.82 (dt, J=10.1, 1.7 Hz, 1H), 7.78 (d, J=4 Hz, 1H), 7.49 (t, J=7.7 Hz, 1H), 6.72 (d, J=2.2 Hz, 1H), 5.72 (bs, 1H), 5.03 (q, J=6.6 Hz, 1H), 4.38 (q, J=7.1 Hz, 2H), 4.30 (bs, 4H), 3.92 (s, 3H), 1.81-1.49 (m, 9H), 1.44 (t, J=7.1 Hz, 3H); LCMS (ESI) m/z: 432.1 [M+H]⁺.

### Synthesis of 2-(9-ethyl-6-morpholino-2-(4-phenyl-1H-pyrazol-1-yl)-9H-purin-8-yl)propan-1-ol (Compound 340)

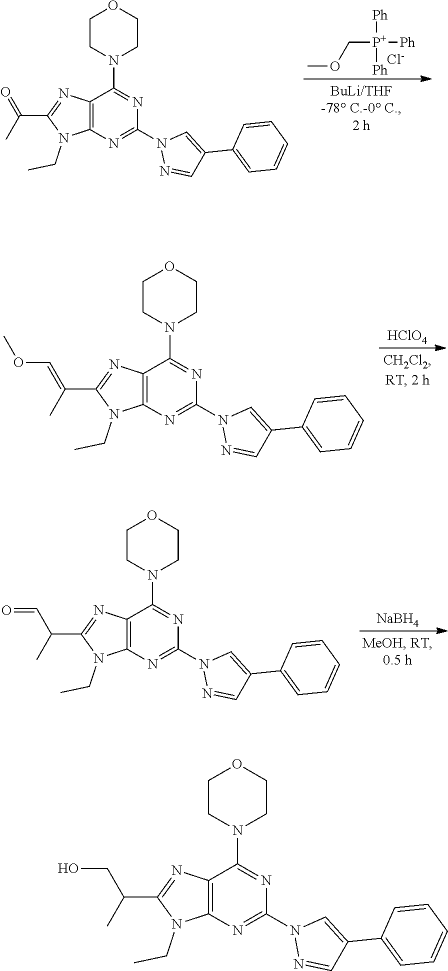

### Step 1: Synthesis of 4-(9-ethyl-8-(1-methoxyprop-1-en-2-yl)-2-(4-phenyl-1H-pyrazol-1-yl)-9H-purin-6-yl)morpholine

To a mixture of (methoxymethyl)triphenylphosphonium chloride (376 mg, 1.1 mmol) in tetrahydrofuran (10 m) at −78° C. was added n-butyllithium (2.5 M in tetrahydrofuran, 0.6 mL, 1.44 mmol) drop-wise. After the addition, the mixture was warmed up and stirred at 0° C. for another 0.5 h. To the resultant mixture was added a solution of 1-(9-ethyl-6-morpholino-2-(4-phenyl-1H-pyrazol-1-yl)-9H-purin-8-yl)ethanone (300 mg, 0.72 mmol) in tetrahydrofuran (5 mL) and stirred for another 2 h. The reaction was then quenched with saturated aqueous ammonium chloride solution extracted with ethyl acetate (150 mL*2). The combined organic phase was concentrated and purified by silica gel column chromatography (50% ethyl acetate in petroleum ether) to afford 4-(9-ethyl-8-(1-methoxyprop-1-en-2-yl)-2-(4-phenyl-1H-pyrazol-1-yl)-9H-purin-6-yl)morpholine (450 mg) as light yellow oil. LCMS (ESI) m/z: 445.8 [M+H]⁺.

### Step 2: Synthesis of 2-(9-ethyl-6-morpholino-2-(4-phenyl-1H-pyrazol-1-yl)-9H-purin-8-yl)propanal

A mixture of 4-(9-ethyl-8-(1-methoxyprop-1-en-2-yl)-2-(4-phenyl-1H-pyrazol-1-yl)-9H-purin-6-yl)morpholine (450 mg, crude), perchloric acid (5 mL) and dichloromethane (20 mL) was stirred at 20° C. for 2 h. It was poured into crushed ice and extracted with dichloromethane (100 mL*2). The organic phase was dried over sodium sulfate and concentrated to afford 2-(9-ethyl-6-morpholino-2-(4-phenyl-1H-pyrazol-1-yl)-9H-purin-8-yl)propanal (250 mg) as light yellow oil, which was used in the next step without further purification. LCMS (ESI) m/z: 432.1 [M+H]⁺.

### Step 3: Synthesis of 2-(9-ethyl-6-morpholino-2-(4-phenyl-1H-pyrazol-1-yl)-9H-purin-8-yl)propan-1-ol

A mixture of 2-(9-ethyl-6-morpholino-2-(4-phenyl-1H-pyrazol-1-yl)-9H-purin-8-yl)propanal (200 mg, crude) and sodium borohydride (76 mg, 2.0 mmol) in methanol (10.0 mL) was stirred at 20° C. for 0.5 h. The mixture was concentrated and the residue was subjected sequentially to silica gel column chromatography (10% methanol in dichloromethane) and prep-HPLC (Column Xbridge 21.2*250 mm C18, 10 um, Mobile Phase A: water (10 mmol/L ammonium bicarbonate) B: acetonitrile) to afford 2-(9-ethyl-6-morpholino-2-(4-phenyl-1H-pyrazol-1-yl)-9H-purin-8-yl)propan-1-ol (14.3 mg, 4.6% over three steps) as a white solid. ¹H NMR (400 MHz, DMSO-d₆) δ 9.05 (d, J=0.6 Hz, 1H), 8.22 (d, J=0.6 Hz, 1H), 7.83-7.74 (m, 2H), 7.41 (t, J=7.7 Hz, 2H), 7.27 (t, J=7.4 Hz, 1H), 4.89 (t, J=5.5 Hz, 1H), 4.48-4.10 (m, 6H), 3.82-3.73 (m, 4H), 3.70-3.59 (m, 2H), 3.28-3.22 (m, 1H), 1.37 (t, J=7.2 Hz, 3H), 1.29 (d, J=6.8 Hz, 3H); LCMS (ESI) m/z: 434.3 [M+H]⁺.

### Synthesis of (9-ethyl-6-morpholino-2-(4-phenyl-1H-pyrazol-1-yl)-9H-purin-8-yl)(4-hydroxypiperidin-1-yl)methanone (Compound 341): 0

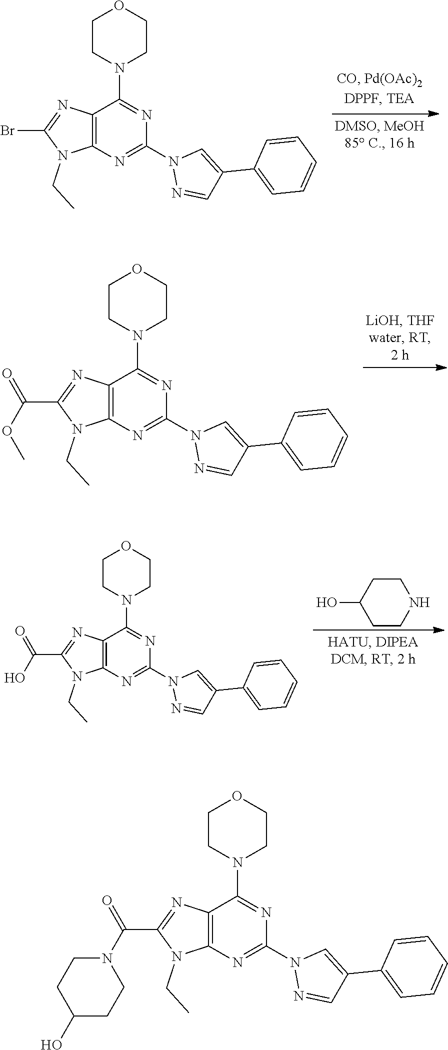 [SMILES: CCn1c(Br)nc2c(N3CCOCC3)nc(-n3cc(-c4ccccc4)cn3)nc21 ; CCn1c(C(=O)OC)nc2c(N3CCOCC3)nc(-n3cc(-c4ccccc4)cn3)nc21 ; CCn1c(C(=O)O)nc2c(N3CCOCC3)nc(-n3cc(-c4ccccc4)cn3)nc21 ; OC1CCNCC1 ; CCn1c(C(=O)N2CCC(O)CC2)nc2c(N3CCOCC3)nc(-n3cc(-c4ccccc4)cn3)nc21]

### Step 1: Synthesis of methyl 9-ethyl-6-morpholino-2-(4-phenyl-1H-pyrazol-1-yl)-9H-purine-8-carboxylate

To a solution of 4-(8-bromo-9-ethyl-2-(4-phenyl-1H-pyrazol-1-yl)-9H-purin-6-yl)morpholine (200 mg, 0.44 mmol) in dimethyl sulfoxide (4 mL) and methanol (5 mL) were added palladium (II) acetate (10 mg, 0.044 mmol), 1,1′-bis(diphenylphosphino) ferrocene (122 mg, 0.22 mmol) and triethylamine (133 mg, 1.32 mmol) and the resultant reaction mixture was stirred at 85° C. for 16 h under carbon monoxide atmosphere. The mixture was then extracted with dichloromethane (20 mL*2) and washed with water (10 mL*2). The organic layer was dried over sodium sulfate, and concentrated. The resultant residue was subjected to silica gel column chromatography (3% methanol in dichloromethane) to obtain methyl 9-ethyl-6-morpholino-2-(4-phenyl-1H-pyrazol-1-yl)-9H-purine-8-carboxylate as white solid (160 mg, 84%). LCMS (ESI) m/z: 434.1 [M+H]⁺

### Step 2: Synthesis of 9-ethyl-6-morpholino-2-(4-phenyl-1H-pyrazol-1-yl)-9H-purine-8-carboxylic acid

To a solution of methyl 9-ethyl-6-morpholino-2-(4-phenyl-1H-pyrazol-1-yl)-9H-purine-8-carboxylate (160 mg, 0.36 mmol) in tetrahydrofuran (2 mL) and water (2 mL) was added lithium hydroxide monohydrate (31 mg, 0.74 mmol) at 0° C. The resultant reaction mixture was then stirred at room temperature for 2 h and concentrated to remove the organics. The pH of the left-over aqueous phase was then adjusted to ˜6 by progressively adding hydrochloric acid aqueous solution (0.5 N). The resultant precipitate was collected by filtration and dried to obtain 9-ethyl-6-morpholino-2-(4-phenyl-1H-pyrazol-1-yl)-9H-purine-8-carboxylic acid as white solid (120 mg, 78%). LCMS (ESI) m/z: 420.1 [M+H]⁺

### Step 3: Synthesis of (9-ethyl-6-morpholino-2-(4-phenyl-1H-pyrazol-1-yl)-9H-purin-8-yl)(4-hydroxypiperidin-1-yl)methanone

To a stirred mixture of 9-ethyl-6-morpholino-2-(4-phenyl-1H-pyrazol-1-yl)-9H-purine-8-carboxylic acid (80 mg, 0.19 mmol) and 1-(bis(dimethylamino)methylene)-1H-[1,2,3]triazolo[4,5-b]pyridine-1-ium 3-oxide hexafluorophosphate(V) (108 mg, 0.28 mmol) in dichloromethane (3 mL) at 000, were added piperidin-4-ol (28 mg, 0.28 mmol) and N,N-diisopropylethylamine (74 mg, 0.57 mmol). The reaction mixture was then warmed up to room temperature and stirred for 16 h. Water was added to the reaction mixture and was extracted with ethyl acetate (50 mL×3). The organic layer was dried, concentrated and the crude product obtained was subjected to prep-TLC (petroleum ether:ethyl acetate from 20:1 to 1:1) to obtain (9-ethyl-6-morpholino-2-(4-phenyl-1H-pyrazol-1-yl)-9H-purin-8-yl)(4-hydroxypiperidin-1-yl)methanone (42.4 mg, 45%) as white solid. H NMR (400 MHz, DMSO-d) 9.09 (, 1H), 8.26 (, 1H), 7.80 (d, J=7 Hz, 2H), 7.42 (t, J=7.6 Hz, 2H), 7.28 (t, J 7.3 Hz, 1H), 4.85 (bs, 1H), 4.50-4.29 (in, 6H) 4.20-3.78 (1, 9H), 1.84-1.78 (1, 2H), 1.42-1.36 (t, 5H); L CMS (ESI) m/z: 503.3 [M+H]⁺.

| Name | Structure | NMR, MS | # |
|---|---|---|---|
| (9-ethyl-6- morpholino-2-(4- phenyl-1H- pyrazol-1-yl)-9H- purin-8-yl)(3- hydroxypyrrolidin- 1-yl)methanone | 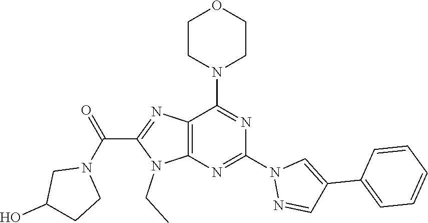 [SMILES: CCn1c(C(=O)N2CCC(O)C2)nc2c(N3CCOCC3)nc(-n3cc(-c4ccccc4)cn3)nc21] | ¹H NMR (400 MHz, DMSO-d₆) δ 9.10 (s, 1H), 8.27 (s, 1H), 7.80 (d, J = 7.3 Hz, 2H), 7.42 (t, J = 7.7 Hz, 2H), 7.29 (d, J = 7.4 Hz, 1H), 5.02 (bs, 1H), 4.60-4.25 (m, 6H), 4.21-4.08 (m, 1H), 3.88 (dd, J = 15.0, 7.9 Hz, 2H), 3.80 (s, 4H), 3.63 (dd, J = 18.1, 9.8 Hz, 2H), 1.96 (bs, 1H), 1.87 (bs, 1H), 1.39 (t, J = 7.0 Hz, 3H). LCMS (ESI) m/z: 488.8 [M]⁺. | 342 |
| (9-ethyl-6- morpholino-2-(4- phenyl-1H- pyrazol-1-yl)-9H- purin-8-yl)(3- hydroxyazetidin- 1-yl)methanone | 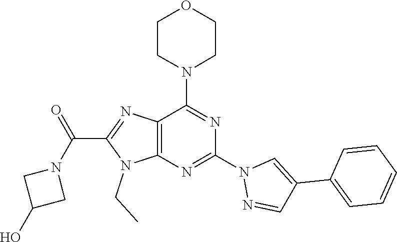 [SMILES: CCn1c(C(=O)N2CC(O)C2)nc2c(N3CCOCC3)nc(-n3cc(-c4ccccc4)cn3)nc21] | ¹H NMR (400 MHz, DMSO-d₆) δ 9.10 (s, 1H), 8.27 (s, 1H), 7.80 (d, J = 7.2 Hz, 2H), 7.42 (t, J = 7.7 Hz, 2H), 7.28 (t, J = 7.4 Hz, 1H), 5.82 (d, J = 6.4 Hz, 1H), 4.82-4.76 (m, 1H), 4.62-4.55 (m, 4H), 4.31 (dd, J = 10.8, 5.8 Hz, 2H), 4.12 (bs, 2H), 3.85-3.79 (m, 6H), 1.37 (t, J = 7.0 Hz, 3H); LCMS (ESI) m/z: 474.3 [M]⁺. | 343 |

### Synthesis of 1-(9-ethyl-2-(3-methoxy-4-phenyl-1H-pyrazol-1-yl)-6-morpholino-9H-purin-8-yl)pyrrolidin-3-ol (Compound 344)

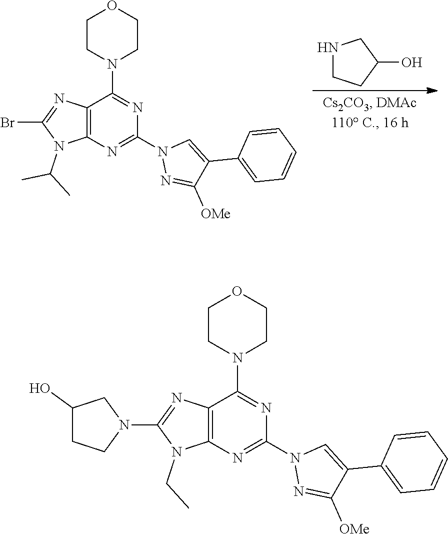 [SMILES: COc1nn(-c2nc(N3CCOCC3)c3nc(Br)n(C(C)C)c3n2)cc1-c1ccccc1 ; OC1CCNC1 ; CCn1c(N2CCC(O)C2)nc2c(N3CCOCC3)nc(-n3cc(-c4ccccc4)c(OC)n3)nc21]

To a solution of 4-(8-bromo-9-ethyl-2-(3-methoxy-4-phenyl-1H-pyrazol-1-yl)-9H-purin-6-yl)morpholine (80 mg, 0.165 mmol) in DMAc (3 mL) were added pyrrolidin-3-ol (22 mg, 0.247 mmol) and cesium carbonate (108 mg, 0.330 mmol). The resultant mixture was stirred at 110° C. for 16 h. The mixture was then filtered and the filtrated was subjected to prep-HPLC (BOSTON pHlex ODS 10 μm 21.2×250 mm 120 A, with mobile phase acetonitrile/0.1% ammonium bicarbonate) to obtain 1-(9-ethyl-2-(3-methoxy-4-phenyl-1H-pyrazol-1-yl)-6-morpholino-9H-purin-8-yl)pyrrolidin-3-ol (33.2 mg, 41%) as white solid. ¹H NMR (400 MHz, DMSO-d₆) δ 8.85 (s, 1H), 7.79 (d, J=7.4 Hz, 2H), 7.39 (t, J=7.7 Hz, 2H), 7.23 (t, J=7.4 Hz, 1H), 5.02 (bs, 1H), 4.39 (s, 1H), 4.26-4.15 (m, 6H), 4.06 (s, 3H), 3.77-3.73 (m, 4H), 3.70 (dd, J=9.9, 5.4 Hz, 2H), 3.57 (td, J=8.7, 3.4 Hz, 1H), 3.40 (s, 1H), 2.01 (dt, J=17.2, 6.4 Hz, 1H), 1.88 (s, 1H), 1.31 (t, J=7.1 Hz, 3H); LCMS (ESI) m/z: 491.1 [M+H]⁺.

### Synthesis of (9-ethyl-2-(3-(1-methyl-1H-pyrazol-3-yl)phenyl)-6-morpholino-9H-purin-8-yl)(3-hydroxyazetidin-1-yl)methanone (Compound 345)

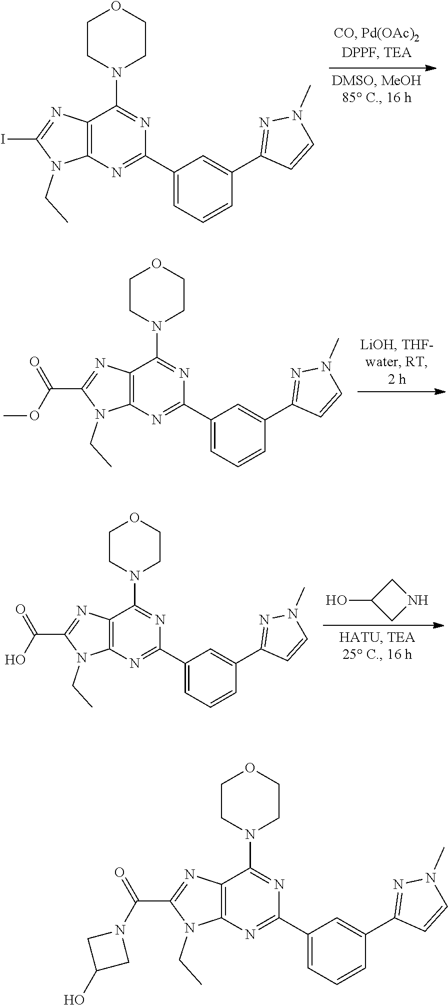

### Step 1: Synthesis of methyl 9-ethyl-2-(3-(1-methyl-1H-pyrazol-3-yl)phenyl)-6-morpholino-9H-purine-8-carboxylate

To a solution of 4-(9-ethyl-8-iodo-2-(3-(1-methyl-1H-pyrazol-3-yl)phenyl)-9H-purin-6-yl)morpholine (500 mg, 0.97 mmol) in dimethyl sulfoxide (40 mL) and methanol (50 mL) were added palladium (II) acetate (21.8 mg, 0.01 mmol), triethylamine (294.7 mg, 2.91 mmol) and 1,1′-bis(diphenylphosphino)ferrocene (107.6 mg, 0.19 mmol) at 25° C., the reaction mixture was heated up and stirred at 85° C. for 16 h under carbon monoxide atmosphere. The resultant mixture was extracted with ethyl acetate (20 mL*2), the combined organic layers was washed with water (10 mL*2), dried over sodium sulfate, and concentrated. The residue was then subjected to silica gel column chromatography (40% ethyl acetate in petroleum ether) to obtain methyl 9-ethyl-2-(3-(1-methyl-1H-pyrazol-3-yl)phenyl)-6-morpholino-9H-purine-8-carboxylate as white solid (400 mg, 92.1%). LCMS (ESI) m/z: 448.3 [M+H]⁺.

### Step 2: Synthesis of 9-ethyl-2-(3-(1-methyl-1H-pyrazol-3-yl)phenyl)-6-morpholino-9H-purine-8-carboxylic acid

To a solution of methyl 9-ethyl-2-(3-(1-methyl-1H-pyrazol-3-yl)phenyl)-6-morpholino-9H-purine-8-carboxylate (400 mg, 0.89 mmol) in tetrahydrofuran (15 mL) and water (4 mL) was added lithium hydroxide hydrate (70 mg, 1.79 mmol) at 0° C., the reaction mixture was warmed up and stirred at 25° C. for 2 h under nitrogen protection. The pH of the resultant mixture was adjusted to ˜3 with hydrochloric acid and the formed precipitate was collected by filtration and dried to obtain 9-ethyl-2-(3-(1-methyl-1H-pyrazol-3-yl)phenyl)-6-morpholino-9H-purine-8-carboxylic acid as white solid (350 mg, 90.3%). LCMS (ESI) m/z: 434.4 [M+H]⁺.

### Step 3: Synthesis of (9-ethyl-2-(3-(1-methyl-1H-pyrazol-3-yl)phenyl)-6-morpholino-9H-purin-8-yl)(3-hydroxyazetidin-1-yl)methanone

To a solution of 9-ethyl-2-(3-(1-methyl-1H-pyrazol-3-yl)phenyl)-6-morpholino-9H-purine-8-carboxylic acid (100 mg, 0.23 mmol) in dichloromethane (10 mL) were added azetidin-3-ol (37.9 mg, 0.35 mmol), benzotriazole-1-yl-oxytripyrrolidinophosphonium hexafluorophosphate (156.0 mg, 0.30 mmol) and triethylamine (70.0 mg, 0.69 mmol) and the reaction mixture was stirred at 25° C. for 16 h under nitrogen protection. The mixture was then extracted with ethyl acetate (2 mL*2), the combined organic layer was washed with water (10 mL*2), dried over sodium sulfate, and concentrated. The residue remained was subjected to silica gel column chromatography (10% methanol in dichloromethane) to obtain (9-ethyl-2-(3-(1-methyl-1H-pyrazol-3-yl)phenyl)-6-morpholino-9H-purin-8-yl)(3-hydroxyazetidin-1-yl)methanone as white solid (39.4 mg, 50%). ¹H NMR (400 MHz, DMSO-d₆) δ 8.77 (s, 1H), 8.33 (d, J 7.9 Hz, 1H), 7.87 (d, J=7.6 Hz, 1H), 7.78 (d, J=1.9 Hz, 1H), 7.51 (t, J=7.7 Hz, 1H), 6.75 (d, J 2.0 Hz, 1H), 5.80 (d, J 5.5 Hz, 1H), 4.85-4.75 (m, 1H), 4.70 (q, J=7.0 Hz, 2H), 4.55 (H, 1H), 4.40-4.20 (, 6H), 3.92 (, 3H), 3.85-3.65 (m, 5H), 1.40 (t, J=7.0 Hz, 3H). LCMS (ESI) m/z: 489.4 [M+H]2.

The following compounds were synthesized according to the protocol described above:

| Name | Structure | NMR, MS | # |
|---|---|---|---|
| (9-ethyl-2-(3-(1- methyl-1H- pyrazol-3- yl)phenyl)-6- morpholino-9H- purin-8- yl)(pyrrolidin-1- yl)methanone | 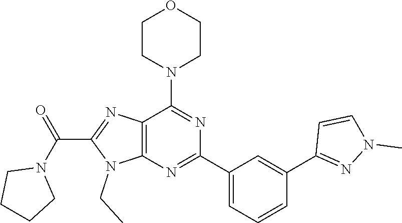 [SMILES: CCn1c(C(=O)N2CCCC2)nc2c(N3CCOCC3)nc(-c3cccc(-c4ccn(C)n4)c3)nc21] | ¹H NMR (500 MHz, DMSO-d₆) δ 8.77 (s, 1H), 8.33 (d, J = 7.8 Hz, 1H), 7.87 (d, J = 7.7 Hz, 1H), 7.78 (d, J = 2.2 Hz, 1H), 7.51 (t, J = 7.7 Hz, 1H), 6.75 (d, J = 2.2 Hz, 1H), 4.51 (q, J = 7.0 Hz, 2H), 4.32 (bs, 4H), 3.92 (s, 3H), 3.80 (dd, J = 11.2, 5.9 Hz, 6H), 3.56 (t, J = 6.4 Hz, 2H), 1.94-1.88 (m, 4H), 1.42 (t, J = 7.1 Hz, 3H). LC-MS (ESI): 486.8 [M]⁺. | 346 |
| N-cyclopropyl-9- ethyl-2-(3-(1- methyl-1H- pyrazol-3- yl)phenyl)-6- morpholino-9H- purine-8- carboxamide | 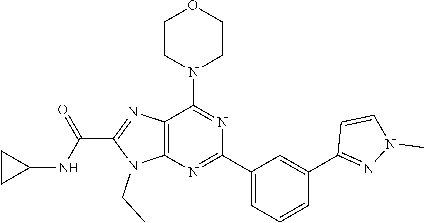 [SMILES: CCn1c(C(=O)NC2CC2)nc2c(N3CCOCC3)nc(-c3cccc(-c4ccn(C)n4)c3)nc21] | ¹H NMR (400 MHz, DMSO-d₆) δ 8.81 (d, J = 4.1 Hz, 1H), 8.77 (s, 1H), 8.33 (d, J = 7.8 Hz, 1H), 7.87 (d, J = 7.8 Hz, 1H), 7.78 (d, J = 2.2 Hz, 1H), 7.51 (t, J = 7.7 Hz, 1H), 6.75 (d, J = 2.2 Hz, 1H), 4.69 (q, J = 7.1 Hz, 2H), 4.35 (bs, 4H), 3.92 (s, 3H), 3.84-3.77 (m, 4H), 2.86 (dt, J = 11.2, 5.5 Hz, 1H), 1.40 (t, J = 7.1 Hz, 3H), 0.79-0.73 (m, 2H), 0.72 (d, J = 4.5 Hz, 2H). LCMS (ESI) m/z: 473.4 [M + H]⁺. | 347 |

### Synthesis of (9-ethyl-2-(3-(I-methyl-1H-pyrazol-3-yl)phenyl)-6-morpholino-9H-purin-8-yl)(pyridin-4-yl)methanol (Compound 348): 0

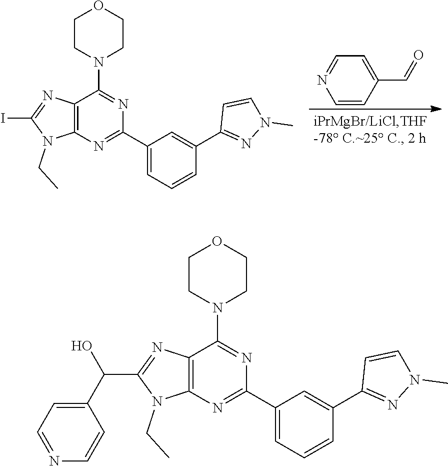 [SMILES: CCn1c(I)nc2c(N3CCOCC3)nc(-c3cccc(-c4ccn(C)n4)c3)nc21 ; O=Cc1ccncc1 ; CCn1c(C(O)c2ccncc2)nc2c(N3CCOCC3)nc(-c3cccc(-c4ccn(C)n4)c3)nc21]

Isopropylmagnesium bromide (1.3M in THE, 0.63 mL, 0.815 mmol) was added to a solution of 4-(9-ethyl-8-iodo-2-(3-(1-methyl-1H-pyrazol-3-yl)phenyl)-9H-purin-6-yl)morpholine (100 mg, 0.194 mmol) in anhydrous THE (3 mL) at −78° C. under nitrogen atmosphere. The reaction mixture was then stirred at −78° C. for 0.5 h followed by the addition of isonicotinaldehyde (83 mg, 0.776 mmol) in THE (1.0 mL) at the same temperature. The resultant reaction mixture was warmed up and stirred at 25° C. for 2 h. The reaction was then quenched by adding aqueous ammonium chloride solution (5 mL) and the mixture was extracted with ethyl acetate (20 mL*3). The combined organic layer was washed with brine (20 mL), dried over sodium sulphate, filtered and concentrated. The residue was subjected to prep-HPLC (BOSTON pHlex ODS 10 μm 21.2×250 mm 120 A, with mobile phase acetonitrile/0.1% ammonium bicarbonate) to obtain (9-ethyl-2-(3-(1-methyl-1H-pyrazol-3-yl)phenyl)-6-morpholino-9H-purin-8-yl)(pyridin-4-yl)methanol (6 mg, 6%) as white solid. ¹H NMR (400 MHz, DMSO-d₆) δ 8.72 (s, 1H), 8.58 (dd, J=4.5, 1.5 Hz, 2H), 8.29 (d, J=7.9 Hz, 1H), 7.84 (d, J=7.7 Hz, 1H), 7.77 (d, J=2.2 Hz, 1H), 7.48 (t, J=7.7 Hz, 1H), 7.42 (d, J=5.5 Hz, 2H), 6.94 (d, J=5.0 Hz, 1H), 6.72 (d, J=2.2 Hz, 1H), 6.12 (d, J=4.9 Hz, 1H), 4.42-4.27 (m, 4H), 4.18 (q, J=7.1 Hz, 2H), 3.91 (s, 3H), 3.85-3.72 (m, 4H), 1.15 (t, J=7.1 Hz, 3H). LCMS (ESI) m/z: 497.1 [M+H]⁺.

### Synthesis of 1-(2-(3-(1H-pyrazol-1-yl)phenyl)-9-ethyl-6-morpholino-9H-purin-8-yl)ethan-1-ol (Compound 349)

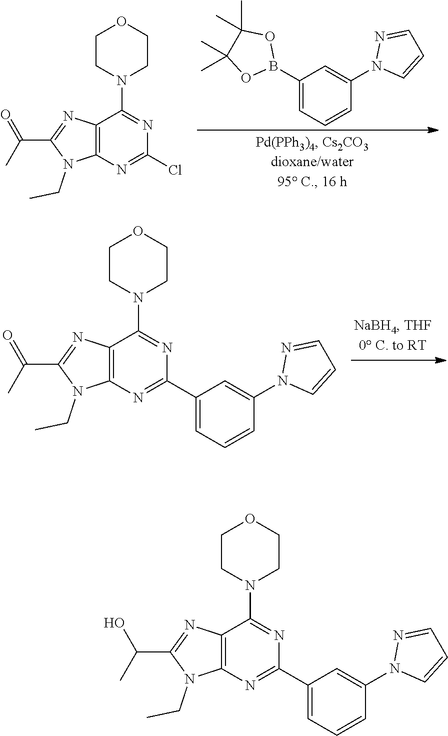 [SMILES: CCn1c(C(C)=O)nc2c(N3CCOCC3)nc(Cl)nc21 ; CC1(C)OB(c2cccc(-n3cccn3)c2)OC1(C)C ; CCn1c(C(C)=O)nc2c(N3CCOCC3)nc(-c3cccc(-n4cccn4)c3)nc21 ; CCn1c(C(C)O)nc2c(N3CCOCC3)nc(-c3cccc(-n4cccn4)c3)nc21]

### Step 1: Synthesis of 1-(2-(3-(1H-pyrazol-1-yl)phenyl)-9-ethyl-6-morpholino-9H-purin-8-yl)ethan-1-one

A mixture of 1-(2-chloro-9-ethyl-6-morpholino-9H-purin-8-yl)ethan-1-one (186 mg, 0.6 mmol), 1-(3-(4,4,5,5-tetramethyl-1,3,2-dioxaborolan-2-yl)phenyl)-1H-pyrazole (194 mg, 0.72 mmol), tetrakis(triphenylphosphine)palladium(0) (138 mg, 0.12 mmol) and cesium carbonate (588 mg, 1.8 mmol) in N,N-dimethylacetamide (10 mL) and water (2 mL) was stirred at 95° C. under argon atmosphere for 16 h. The reaction mixture was concentrated and the residue was subjected to silica gel column chromatography (petroleum ether:ethyl acetate=85:15) to obtain 1-(2-(3-(1H-pyrazol-1-yl)phenyl)-9-ethyl-6-morpholino-9H-purin-8-yl)ethan-1-one as white solid. (208 mg, 83%). LCMS (ESI) m/z: 418.3 [M+H]⁺.

### Step 2: Synthesis of 1-(2-(3-(1H-pyrazol-1-yl)phenyl)-9-ethyl-6-morpholino-9H-purin-8-yl)ethan-1-ol

Sodium borohydride (38 mg, 1 mmol) was added portion-wise to a solution of 1-(2-(3-(1H-pyrazol-1-yl)phenyl)-9-ethyl-6-morpholino-9H-purin-8-yl)ethan-1-one (208 mg, 0.5 mmol) in tetrahydrofuran (5 mL) at 0° C., and the resultant mixture was warmed up and stirred at room temperature for 1 h. It was then filtered and the filtrate was subjected to prep-HPLC (Column Xbridge 21.2*250 mm C18, 10 um, Mobile Phase A: water (10 mmol/L ammonium bicarbonate) B: acetonitrile) to obtain 1-(2-(3-(1H-pyrazol-1-yl)phenyl)-9-ethyl-6-morpholino-9H-purin-8-yl)ethan-1-ol (165.1 mg, 80%) as white solid. ¹H NMR (400 MHz, DMSO-d₆) δ 8.79 (s, 1H), 8.57 (d, J=2.4 Hz, 1H), 8.34 (d, J=7.8 Hz, 1H), 7.89 (dd, J=8.0, 1.4 Hz, 1H), 7.81 (d, J=1.5 Hz, 1H), 7.60 (t, J=7.9 Hz, 1H), 6.59 (t, J=4.0 Hz, 1H), 5.72 (s, 1H), 5.04 (q, J=6.5 Hz, 1H), 4.42 (q, J=7.1 Hz, 2H), 4.32 (bs, 4H), 3.84-3.71 (m, 4H), 1.58 (d, J=6.5 Hz, 3H), 1.44 (t, J=7.1 Hz, 3H). LCMS (ESI) m/z: 420.3 [M+H]⁺.

### Synthesis of enantiomer 1 (Compound 350) and enantiomer 2 (Compound 351) of 1-(9-ethyl-2-(3-(1-methyl-1H-pyrazol-3-yl)phenyl)-6-morpholino-9H-purin-8-yl)ethane-1,2-diol

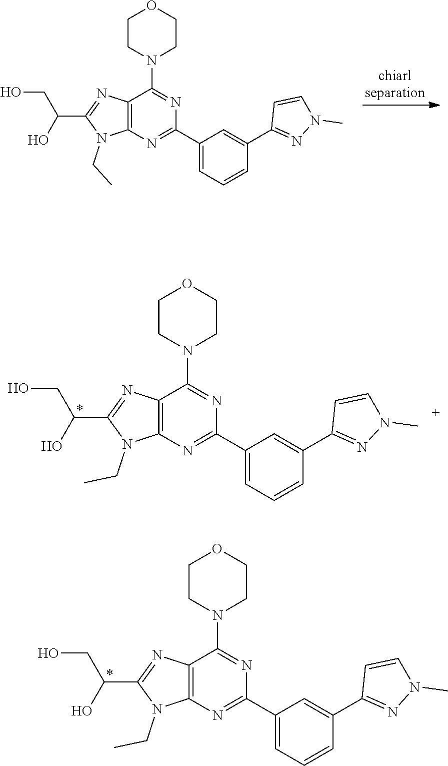 [SMILES: CCn1c(C(O)CO)nc2c(N3CCOCC3)nc(-c3cccc(-c4ccn(C)n4)c3)nc21 ; CCn1c(C(O)CO)nc2c(N3CCOCC3)nc(-c3cccc(-c4ccn(C)n4)c3)nc21 ; CCn1c(C(O)CO)nc2c(N3CCOCC3)nc(-c3cccc(-c4ccn(C)n4)c3)nc21]

The racemic compound was chirally resolved by employing prep-HPLC conditions described earlier. The enantiomer 1 (Compound 12) and enantiomer 2 (Compound 13) were isolated as white solids.

Compound 12: ¹H NMR (400 MHz, DMSO-d₆) δ 8.75 (s, 1H), 8.31 (d, J=7.8 Hz, 1H), 7.85 (d, J=7.7 Hz, 1H), 7.78 (d, J=2.1 Hz, 1H), 7.49 (t, J=7.7 Hz, 1H), 6.74 (d, J=2.2 Hz, 1H), 5.85 (d, J=5.7 Hz, 1H), 4.87 (q, J=5.6 Hz, 2H), 4.42 (q, J=7.0 Hz, 2H), 4.30 (bs, 4H), 3.98-3.73 (m, 9H), 1.44 (t, J=7.0 Hz, 3H); LCMS (ESI) m/z: 450.4 [M+H]⁺; (Rt: 1.543 min).

Compound 13: ¹H NMR (400 MHz, DMSO-d₆) δ 8.75 (s, 1H), 8.31 (d, J=7.8 Hz, 1H), 7.85 (d, J=7.8 Hz, 1H), 7.78 (d, J=2.0 Hz, 1H), 7.49 (t, J=7.7 Hz, 1H), 6.74 (d, J=2.1 Hz, 1H), 5.85 (d, J=5.7 Hz, 1H), 4.87 (q, J=5.2 Hz, 2H), 4.40 (q, J=7.0 Hz, 2H), 4.30 (bs, 4H), 3.94-3.75 (m, 9H), 1.43 (t, J=7.0 Hz, 3H); LCMS (ESI) m/z: 450.3 [M+H]⁺; (Rt: 1.89 min).

### Synthesis of enantiomer 1 (Compound 352) and enantiomer 2 (Compound 353) of 1-(9-ethyl-2-(3-(1-methyl-1H-pyrazol-3-yl)phenyl)-6-morpholino-9H-purin-8-yl)ethan-1-ol

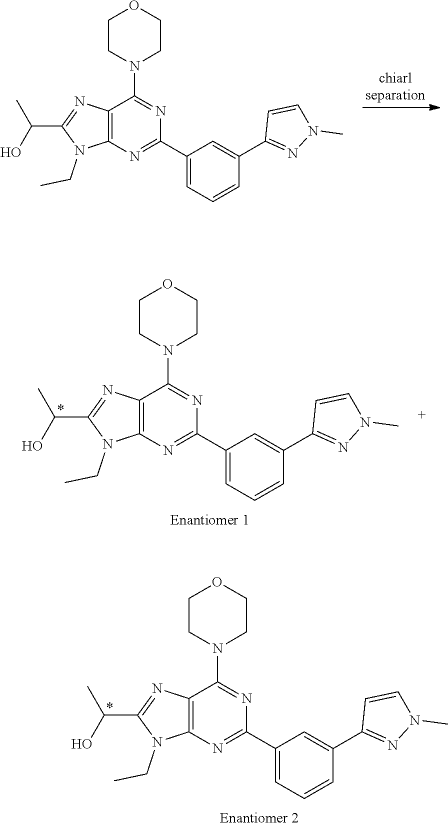 [SMILES: CCn1c(C(C)O)nc2c(N3CCOCC3)nc(-c3cccc(-c4ccn(C)n4)c3)nc21 ; CCn1c(C(C)O)nc2c(N3CCOCC3)nc(-c3cccc(-c4ccn(C)n4)c3)nc21 ; CCn1c(C(C)O)nc2c(N3CCOCC3)nc(-c3cccc(-c4ccn(C)n4)c3)nc21]

Compound 14 and 15 were similarly isolated from the corresponding racemic compound. Compound 14: ¹H NMR (400 MHz, DMSO-d₆) δ 8.75 (s, 1H), 8.31 (d, J=7.8 Hz, 1H), 7.84 (d, J=7.8 Hz, 1H), 7.78 (d, J=2.1 Hz, 1H), 7.49 (t, J=7.7 Hz, 1H), 6.74 (d, J=2.2 Hz, 1H), 6.14-5.23 (m, 1H), 5.04 (q, J=6.6 Hz, 1H), 4.40 (q, J=7.1 Hz, 2H), 4.31 (bs, 4H), 3.92 (s, 3H), 3.78 (t, J=4.4 Hz, 4H), 1.58 (d, J=6.5 Hz, 3H), 1.44 (t, J=7.1 Hz, 3H); LCMS (ESI) m/z: 434.4 [M+H]⁺; (Rt: 2.905 min).

Compound 15: ¹H NMR (400 MHz, DMSO-d₆) δ 8.76 (s, 1H), 8.31 (d, J=7.8 Hz, 1H), 7.85 (d, J=7.8 Hz, 1H), 7.78 (d, J=2.0 Hz, 1H), 7.49 (t, J=7.7 Hz, 1H), 6.74 (d, J=2.1 Hz, 1H), 5.71 (d, J=6.1 Hz, 1H), 5.00 (q, J=6.5 Hz, 1H), 4.40 (q, J=7.0 Hz, 2H), 4.31 (bs, 4H), 3.92 (s, 3H), 3.79 (d, J=4.3 Hz, 4H), 1.58 (d, J=6.5 Hz, 3H), 1.44 (t, J=7.0 Hz, 3H); LCMS (ESI) m/z: 434.4 [M+H]⁺; (Rt: 3.314 min).

### Preparation of enantiomer 1 (Compound 354) and enantiomer 2 (Compound 355) of 1-(9-ethyl-2-(3-methoxy-4-phenyl-1H-pyrazol-1-yl)-6-morpholino-9H-purin-8-yl)ethane-1,2-diol

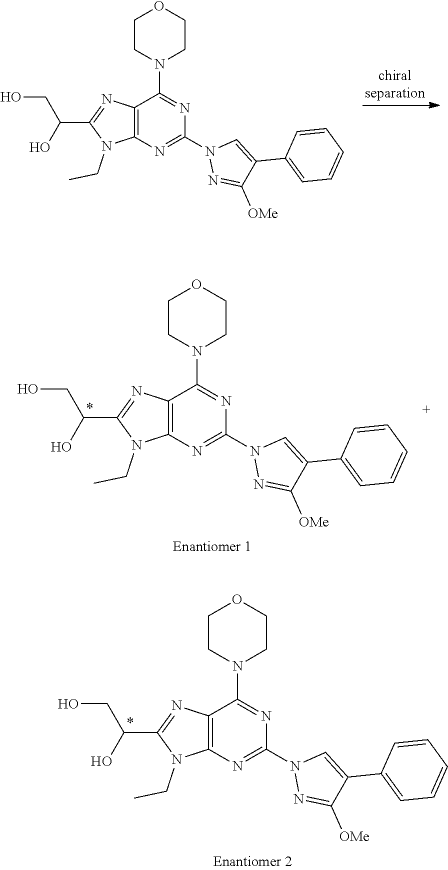 [SMILES: CCn1c(C(O)CO)nc2c(N3CCOCC3)nc(-n3cc(-c4ccccc4)c(OC)n3)nc21 ; CCn1c(C(O)CO)nc2c(N3CCOCC3)nc(-n3cc(-c4ccccc4)c(OC)n3)nc21 ; CCn1c(C(O)CO)nc2c(N3CCOCC3)nc(-n3cc(-c4ccccc4)c(OC)n3)nc21]

The racemic compound was subjected to chiral HPLC conditions [Instrument: SFC-150 (Waters); Column: WHELK 20*250 mm, 10 um (Daicel); Column temperature: 35° C.; Mobile phase: carbon dioxide/ethanol (0.5% methanol ammonia)=50/50; Flow rate: 100 g/min; Back pressure: 100 bar; Detection wavelength: 214 nm; Cycle time: 4.6 min; Sample solution: 40 mg dissolved in 20 ml methanol and dichloromethane; Injection volume: 1.0 ml] to afford compound 16 (15.8 mg, 42.7%) and compound 17 (10.8 mg, 29.2%) as white solids.

Compound 16: ¹H NMR (400 MHz, DMSO-d₆) δ 8.92 (s, 1H), 7.81 (d, J=7.4 Hz, 2H), 7.40 (t, J=7.7 Hz, 2H), 7.25 (d, J=7.4 Hz, 1H), 5.86 (s, 1H), 4.82 (t, J=6.2 Hz, 2H), 4.45-4.30 (m, 6H), 4.07 (s, 3H), 392-3.68 (m, 6H), 1.40 (t, J=7.1 Hz, 3H). LCMS (ESI) m/z: 466.3 [M+H]⁺; (Rt: 1.824 min).

Compound 17: ¹H NMR (400 MHz, DMSO-d₆) δ 8.91 (s, 1H), 7.81 (d, J=7.4 Hz, 2H), 7.40 (t, J=7.7 Hz, 2H), 7.25 (t, J=7.3 Hz, 1H), 5.83 (d, J=5.5 Hz, 1H), 4.85-4.73 (m, 2H), 4.47-4.11 (m, 6H), 4.07 (s, 3H), 3.85-3.70 (m, 6H), 1.40 (t, J=7.1 Hz, 3H). LCMS (ESI) m/z: 466.3 [M+H]⁺; (Rt: 2.372 min).

### Synthesis of 1-(9-ethyl-2-(3-(1-methyl-1H-pyrazol-3-yl)phenyl)-6-morpholino-9H-purin-8-yl)cyclobutanol (Compound 356)

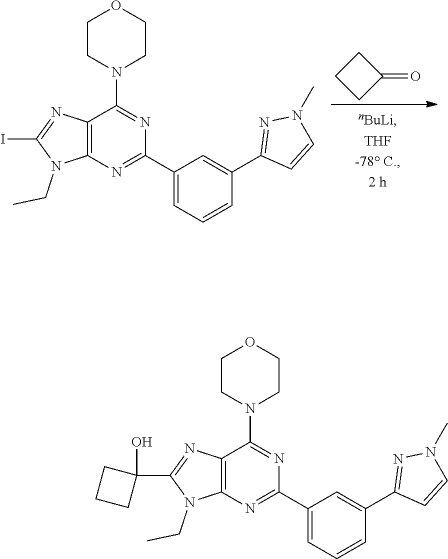

To a stirred solution of 4-(9-ethyl-8-iodo-2-(3-(1-methyl-1H-pyrazol-3-yl)phenyl)-9H-purin-6-yl)morpholine (103 mg, 0.2 mmol) in tetrahydrofuran (5 mL) at −78° C., was added 1.6 M butyl lithium in hexane (0.25 mL, 0.4 mmol) drop-wise and the reaction mixture was stirred for 10 min at that temperature. A solution of cyclobutanone (28 mg, 0.4 mmol) in 1 mL of THE was then added drop-wise to the mixture and it was stirred at −78° C. for 2 h. The reaction was quenched with saturated aqueous ammonium chloride solution (10 mL), then diluted with water (20 mL) and extracted with dichloromethane (50 mL*3). The organic layer was dried over sodium sulfate and concentrated. The resultant residue was subjected to prep-HPLC (BOSTON pHlex ODS 10 μm 21.2×250 mm 120 A. The mobile phase was acetonitrile/0.1% ammonium bicarbonate) to obtain 1-(9-ethyl-2-(3-(1-methyl-1H-pyrazol-3-yl)phenyl)-6-morpholino-9H-purin-8-yl)cyclobutanol (22.3 mg, 16%) as white solid. ¹H NMR (400 MHz, DMSO-d₆) δ 8.75 (s, 1H), 8.31 (d, J=7.9 Hz, 1H), 7.84 (d, J=7.7 Hz, 1H), 7.78 (d, J=2.2 Hz, 1H), 7.49 (t, J=7.7 Hz, 1H), 6.73 (d, J=2.2 Hz, 1H), 6.21 (s, 1H), 4.36-4.31 (m, 6H), 3.92 (s, 3H), 3.84-3.75 (m, 4H), 2.86-2.74 (m, 2H), 2.34 (dt, J=11.9, 9.5 Hz, 2H), 1.93-1.83 (m, 1H), 1.63 (dt, J=10.5, 8.6 Hz, 1H), 1.45 (t, J=7.0 Hz, 3H). LCMS (ESI) m/z: 460.2 [M+H]⁺.

### Synthesis of (2-(3-(1H-pyrazol-1-yl)phenyl)-9-ethyl-6-morpholino-9H-purin-8-yl)(pyridin-4-yl)methanol (Compound 357)

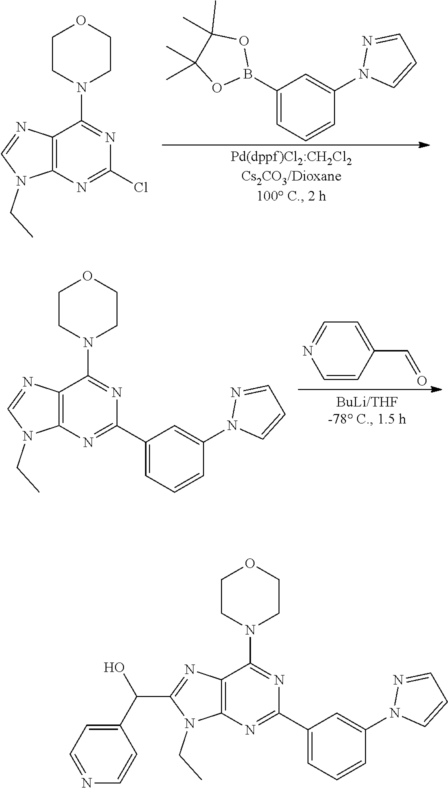 [SMILES: CCn1cnc2c(N3CCOCC3)nc(Cl)nc21 ; CC1(C)OB(c2cccc(-n3cccn3)c2)OC1(C)C ; CCn1cnc2c(N3CCOCC3)nc(-c3cccc(-n4cccn4)c3)nc21 ; O=Cc1ccncc1 ; CCn1c(C(O)c2ccncc2)nc2c(N3CCOCC3)nc(-c3cccc(-n4cccn4)c3)nc21]

### Step 1: Synthesis of 4-(2-(3-(1H-pyrazol-1-yl)phenyl)-9-ethyl-9H-purin-6-yl)morpholine

A mixture of 4-(2-chloro-9-ethyl-9H-purin-6-yl)morpholine (1.0 g, 3.7 mmol), 1-(3-(4,4,5,5-tetramethyl-1,3,2-dioxaborolan-2-yl)phenyl)-1H-pyrazole (1.0 g, 3.7 mmol), 1,1′-bis(diphenylphosphino)ferrocene-palladium(II)dichloride dichloromethane complex (147 mg, 0.18 mmol) and cesium carbonate (2.4 g, 7.4 mmol) in dioxane (25 mL) and water (4 mL) was stirred at 100° C. under nitrogen atmosphere for 2 h. The mixture was then poured into water, extracted with ethyl acetate (150 mL*2) and the combined organic phase was concentrated. The resulting residue was subjected to silica gel column chromatography (10% methanol in dichloromethane) to obtain brown oil, which was triturated with methanol (10 mL) to afford 4-(2-(3-(1H-pyrazol-1-yl)phenyl)-9-ethyl-5,9-dihydro-4H-purin-6-yl)morpholine (500 mg, 35.1%) as white solid. LCMS (ESI) m/z: 375.9 [M]⁺.

### Step 2: Synthesis of (2-(3-(1H-pyrazol-1-yl)phenyl)-9-ethyl-6-morpholino-9H-purin-8-yl)(pyridin-4-yl)methanol

To a solution of 4-(2-(3-(1H-pyrazol-1-yl)phenyl)-9-ethyl-9H-purin-6-yl)morpholine (120 mg, 0.32 mmol) in tetrahydrofuran (5 mL) was added n-butyllithium (2.5 M in tetrahydrofuran, 0.3 mL, 0.75 mmol) drop-wise at −78° C. under nitrogen atmosphere. After the addition, the mixture was stirred for another 30 min, followed by the addition of isonicotinaldehyde (52 mg, 0.48 mmol) in tetrahydrofuran (2 mL) via syringe. The resultant mixture was stirred at −78° C. for another 1 h and then poured into crushed ice and extracted with ethyl acetate (100 mL*2). The combined organic phase was concentrated and the residue was subjected to silica gel column chromatography (10% methanol in dichloromethane) and then to prep-HPLC (Column Xbridge 21.2*250 mm C18, 10 um, Mobile Phase A: water (10 mmol/L ammonium bicarbonate) to obtain (2-(3-(1H-pyrazol-1-yl)phenyl)-9-ethyl-6-morpholino-9H-purin-8-yl)(pyridin-4-yl)methanol (33.8 mg, 21.8%) as light-yellow solid. ¹H NMR (400 MHz, CDCl3) δ 8.68 (s, 1H), 8.62 (d, J=5.9 Hz, 2H), 8.37 (dt, J=8.0, 2.4 Hz, 1H), 8.02 (dd, J=2.4, 0.5 Hz, 1H), 7.78-7.73 (m, 2H), 7.51 (t, J=7.9 Hz, 1H), 7.35 (d, J=5.8 Hz, 2H), 6.50 (dd, J=2.4, 1.9 Hz, 1H), 6.00 (d, J=2.8 Hz, 1H), 4.39 (s, 5H), 4.13 (q, J=7.2 Hz, 2H), 3.94-3.85 (m, 4H), 1.17 (t, J=7.2 Hz, 3H); LCMS (ESI) m/z: 482.7 [M+H]⁺.

### Synthesis of 4-(9-ethyl-2-(3-methoxy-4-phenyl-1H-pyrazol-1-yl)-6-(piperidin-1-yl)-9H-purin-8-yl)-1-methylpiperazin-2-one (Compound 358)

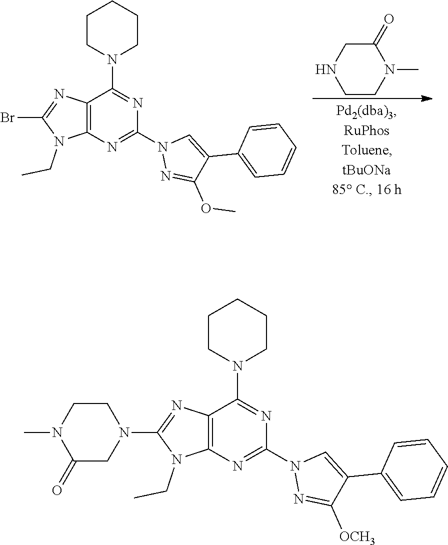

A mixture of 8-bromo-9-ethyl-2-(3-methoxy-4-phenyl-1H-pyrazol-1-yl)-6-(piperidin-1-yl)-9H-purine (150 mg, 0.311 mmol), 1-methylpiperazin-2-one (71 mg, 0.620 mmol), tris(dibenzylidene acetone)dipalladium (30 mg, 0.05 mmol), 2-dicyclohexylphosphino-2′,6′-diisopropoxy-1,1′-biphenyl (56 mg, 0.06 mmol) and sodium tert-butoxide (60 mg, 0.620 mmol) in toluene (10 mL) was stirred at 85° C. for 16 h. The reaction mixture was cooled, then quenched with water (15 mL) and extracted with ethyl acetate (20 mL*3). The organic layer was combined, washed with brine (30 mL), dried over sodium sulfate, filtered and concentrated. The residue was subjected to prep-HPLC (0.05% ammonium bicarbonate:acetonitrile=5%-95%) to obtain 4-(9-ethyl-2-(3-methoxy-4-phenyl-1H-pyrazol-1-yl)-6-(piperidin-1-yl)-9H-purin-8-yl)-1-methylpiperazin-2-one (21.5 mg, 13.4%) as white solid. ¹H NMR (400 MHz, DMSO-d₆) δ 8.84 (s, 1H), 7.80 (d, J=7.2 Hz, 2H), 7.39 (t, J=7.7 Hz, 2H), 7.24 (t, J=7.4 Hz, 1H), 4.20 (bs, 4H), 4.14 (q, J=7.2 Hz, 2H), 4.06 (s, 3H), 3.85 (s, 2H), 3.50 (dd, J=14.2, 5.0 Hz, 4H), 2.91 (s, 3H), 1.80-1.54 (m, 6H), 1.40 (t, J=7.2 Hz, 3H). LCMS (ESI) m/z: 515.7 [M]⁺.

### Synthesis of (S)-8-(9-ethyl-2-(3-(1-methyl-1H-pyrazol-3-yl)phenyl)-6-morpholino-9H-purin-8-yl)octahydropyrazino[2,1-c][1,4]oxazine (Compound 359)

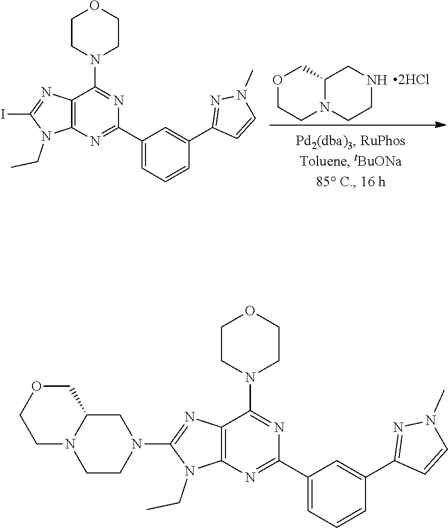

A solution of (4-(9-ethyl-8-iodo-2-(3-(1-methyl-1H-pyrazol-3-yl)phenyl)-9H-purin-6-yl)morpholine (52 mg, 0.1 mmol), (S)-octahydropyrazino[2,1-c][1,4]oxazine dihydrochloride (22 mg, 0.1 mmol), tris(dibenzylideneacetone)dipalladium (18 mg, 0.02 mmol), 2-dicyclohexylphosphino-2′,6′-diisopropoxybiphenyl (19 mg, 0.04 mmol), and sodium tert-butoxide (58 mg, 0.6 mmol) in toluene (7 mL) was stirred at 85° C. for 16 h under argon atmosphere. The reaction mixture was concentrated and the residue was subjected to prep-HPLC (BOSTON pHlex ODS 10 μm 21.2×250 mm 120 A. The mobile phase was acetonitrile/0.1% ammonium bicarbonate) to obtain (S)-8-(9-ethyl-2-(3-(1-methyl-1H-pyrazol-3-yl)phenyl)-6-morpholino-9H-purin-8-yl)octahydropyrazino[2,1-c][1,4]oxazine (11.7 mg, 15%) as white solid. ¹H NMR (400 MHz, DMSO-d₆) δ 8.72 (s, 1H), 8.27 (d, J=7.6 Hz, 1H), 7.82 (d, J=7.6 Hz, 1H), 7.77 (d, J=2.2 Hz, 1H), 7.47 (t, J=7.7 Hz, 1H), 6.73 (d, J=2.2 Hz, 1H), 4.27-4.15 (m, 6H), 3.92 (s, 3H), 3.82-3.72 (m, 6H), 3.58-3.48 (m, 2H), 3.17 (t, J=10.1 Hz, 1H), 3.09-3.02 (m, 1H), 2.83 (d, J=10.0 Hz, 1H), 2.71-2.62 (m, 2H), 2.44-2.22 (m, 4H), 1.44 (t, J=7.2 Hz, 3H). LCMS (ESI) m/z: 529.8 [M]⁺.

The following compound was synthesized according to the above protocol.

| Name | Structure | NMR, MS | # |
|---|---|---|---|
| (R)-8-(9-ethyl-2- (3-(1-methyl-1H- pyrazol-3- yl)phenyl)-6- morpholino-9H- purin-8- yl)octahydro- pyrazino[2,1- c][1,4]oxazine | 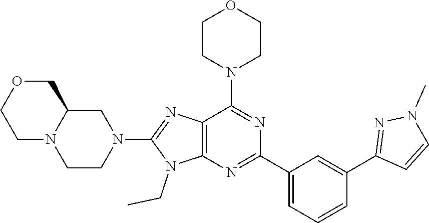 [SMILES: CCn1c(N2CCN3CCOC[C@H]3C2)nc2c(N3CCOCC3)nc(-c3cccc(-c4ccn(C)n4)c3)nc21] | ¹H NMR (400 MHz, DMSO-d₆) δ 8.72 (s, 1H), 8.28 (d, J = 7.9 Hz, 1H), 7.82 (d, J = 7.9 Hz, 1H), 7.77 (d, J = 2.1 Hz, 1H), 7.47 (t, J = 7.7 Hz, 1H), 6.73 (d, J = 2.2 Hz, 1H), 4.29-4.14 (m, 6H), 3.92 (s, 3H), 3.83-3.71 (m, 6H), 3.58-3.47 (m, 2H), 3.17 (t, J = 10.4 Hz, 1H), 3.06 (t, J = 10.8 Hz, 1H), 2.83 (d, J = 11.7 Hz, 1H), 2.71-2.62 (m, 2H), 2.43-2.24 (m, 4H), 1.44 (t, J = 7.1 Hz, 3H). LCMS (ESI) m/z: 529.8 [M + H]⁺. | 360 |

### Synthesis of 8-(9-ethyl-6-((S)-3-methylmorpholino)-2-(4-phenyl-1H-pyrazol-1-yl)-9H-purin-8-yl)octahydropyrazino[2,1-c][1,4]oxazine (Compound 361) and its diastereoisomer 1 (Compound 362) and diastereoisomer 2 (Compound 363)

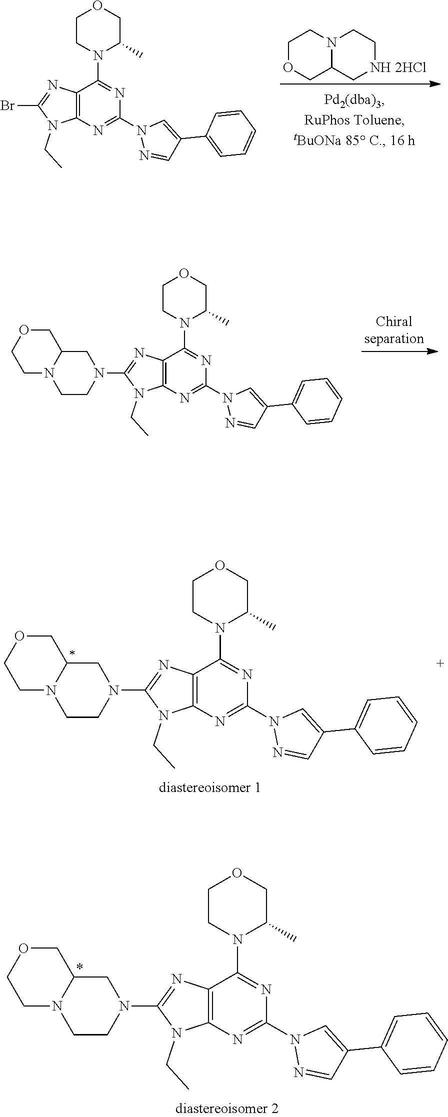 [SMILES: CCn1c(Br)nc2c(N3CCOC[C@@H]3C)nc(-n3cc(-c4ccccc4)cn3)nc21 ; C1CN2CCOCC2CN1 ; CCn1c(N2CCN3CCOCC3C2)nc2c(N3CCOC[C@@H]3C)nc(-n3cc(-c4ccccc4)cn3)nc21 ; CCn1c(N2CCN3CCOCC3C2)nc2c(N3CCOC[C@@H]3C)nc(-n3cc(-c4ccccc4)cn3)nc21 ; CCn1c(N2CCN3CCOCC3C2)nc2c(N3CCOC[C@@H]3C)nc(-n3cc(-c4ccccc4)cn3)nc21]

### Step 1: Synthesis of 8-(9-ethyl-6-((S)-3-methylmorpholino)-2-(4-phenyl-1H-pyrazol-1-yl)-9H-purin-8-yl)octahydropyrazino[2,1-c][1,4]oxazine

A solution of (S)-4-(8-bromo-9-ethyl-2-(4-phenyl-1H-pyrazol-1-yl)-9H-purin-6-yl)-3-methylmorpholine (140 mg, 0.3 mmol), octahydropyrazino[2,1-c][1,4]oxazine dihydrochloride (65 mg, 0.3 mmol), tris(dibenzylideneacetone)dipalladium (55 mg, 0.06 mmol), 2-dicyclohexylphosphino-2′,6′-diisopropoxybiphenyl (56 mg, 0.12 mmol), and sodium tert-butoxide (173 mg, 1.8 mmol) in toluene (15 mL) was stirred at 85° C. for 16 h under argon atmosphere. The reaction mixture was concentrated and the resultant residue was subjected to prep-HPLC (BOSTON pHlex ODS 10 μm 21.2×250 mm 120 A. The mobile phase was acetonitrile/0.1% ammonium bicarbonate) to obtain 8-(9-ethyl-6-((S)-3-methylmorpholino)-2-(4-phenyl-1H-pyrazol-1-yl)-9H-purin-8-yl)octahydropyrazino[2,1-c][1,4]oxazine (86 mg, 46%) as white solid. ¹H NMR (500 MHz, DMSO-d₆) δ 8.98 (s, 1H), 8.20 (s, 1H), 7.77 (d, J=7.6 Hz, 2H), 7.41 (t, J=7.7 Hz, 2H), 7.27 (t, J=7.4 Hz, 1H), 5.37 (bs, 1H), 5.05 (bs, 1H), 4.21-4.04 (m, 2H), 3.99 (dd, J=10.8, 2.6 Hz, 1H), 3.82-3.68 (m, 4H), 3.55 (t, J=11.5 Hz, 2H), 3.50-3.34 (m, 3H), 3.16 (t, J=10.5 Hz, 1H), 3.05 (td, J=11.8, 2.3 Hz, 1H), 2.82 (d, J=11.3 Hz, 1H), 2.69 (d, J=11.5 Hz, 1H), 2.63 (t, J=11.2 Hz, 1H), 2.42 (td, J=11.6, 2.9 Hz, 1H), 2.38-2.33 (m, 1H), 2.28 (td, J=11.5, 3.2 Hz, 1H), 1.41 (t, J=7.2 Hz, 3H), 1.32 (d, J=6.7 Hz, 3H). LCMS (ESI) m/z: 530.3 [M+H]⁺.

### Step 2: Preparation of Compounds 24 and 25

A solution of 8-(9-ethyl-6-((S)-3-methylmorpholino)-2-(4-phenyl-1H-pyrazol-1-yl)-9H-purin-8-yl)octahydropyrazino[2,1-c][1,4]oxazine (80 mg, 0.233 mmol) in methanol (25 mL) was subjected to chiral-HPLC (instrument: SFC-80 (Thar, Waters); column: OZ 20*250 mm, 10 um (Daicel); column temperature: 35° C.; mobile phase: carbon dioxide/methanol (0.2% methanol ammonia)=60/40; flow rate: 80 g/min; back pressure: 100 bar; detection wavelength: 214 nm; cycle time: 5.5 min; sample solution: 80 mg dissolved in 25 mL methanol; injection volume: 1 mL) to obtain two diastereomers.

Compound 24 was isolated (5.0 mg, 6%) isolated as white solid. ¹H NMR (400 MHz, DMSO-d₆) δ 8.99 (s, 1H), 8.21 (s, 1H), 7.77 (d, J=7.2 Hz, 2H), 7.41 (t, J=7.7 Hz, 2H), 7.27 (t, J=7.4 Hz, 1H), 5.37 (s, 1H), 5.03 (s, 1H), 4.13 (dt, J=10.4, 7.2 Hz, 2H), 3.99 (dd, J=11.1, 2.8 Hz, 1H), 3.81-3.68 (m, 4H), 3.59-3.34 (m, 5H), 3.16 (t, J=10.5 Hz, 1H), 3.05 (td, J=12.2, 2.2 Hz, 1H), 2.82 (d, J=11.3 Hz, 1H), 2.73-2.59 (m, 2H), 2.35-2.25 (m, 3H), 1.40 (t, J=7.2 Hz, 3H), 1.32 (d, J=6.7 Hz, 3H). LCMS (ESI) m/z: 529.8 [M]⁺. (Rt: 2.943 min).

Compound 25 was isolated (43.2 mg, 54%) as white solid. ¹H NMR (400 MHz, DMSO-d₆) δ 8.99 (s, 1H), 8.21 (s, 1H), 7.77 (d, J=7.2 Hz, 2H), 7.41 (t, J=7.7 Hz, 2H), 7.27 (t, J=7.3 Hz, 1H), 5.37 (s, 1H), 5.04 (s, 1H), 4.18-4.09 (m, 2H), 3.99 (dd, J=11.2, 2.7 Hz, 1H), 3.81-3.68 (m, 4H), 3.60-3.39 (m, 5H), 3.16 (t, J=10.5 Hz, 1H), 3.04 (td, J=12.0, 2.6 Hz, 1H), 2.82 (d, J=11.1 Hz, 1H), 2.71-2.62 (m, 2H), 2.43-2.25 (m, 3H), 1.40 (t, J=7.1 Hz, 3H), 1.32 (d, J=6.7 Hz, 3H). LCMS (ESI) m/z: 529.8 [M+H]⁺. (Rt: 3.526 min).

### Synthesis of 1-(9-ethyl-8-(1-hydroxyethyl)-2-(3-(1-methyl-1H-pyrazol-3-yl)phenyl)-9H-purin-6-yl)piperidin-4-ol (Compound 364)

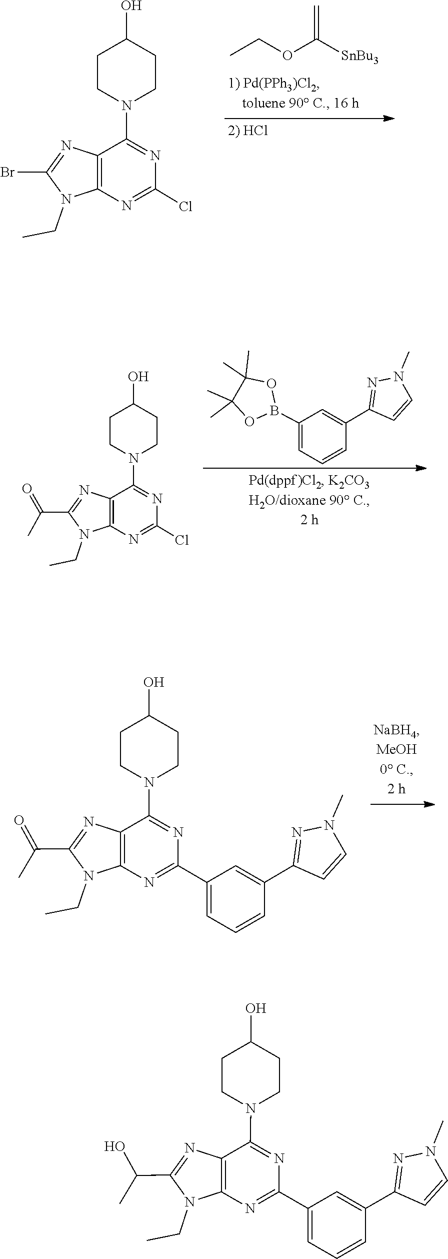

### Step 1: Synthesis of 1-(2-chloro-9-ethyl-6-(4-hydroxypiperidin-1-yl)-9H-purin-8-yl)ethan-1-one

To a solution of 1-(8-bromo-2-chloro-9-ethyl-9H-purin-6-yl)piperidin-4-ol (500.0 mg, 1.39 mmol) in toluene (20 mL) was added tributyl(1-ethoxyvinyl)stannane (554.8 mg, 1.53 mmol) and bis(triphenylphosphine)palladium(II) chloride (101.9 mg, 0.14 mmol) at 25° C. and the reaction mixture was stirred at 90° C. for 16 h under nitrogen atmosphere. To the mixture, was added concentrated HCl (10 mL) and it was further stirred at 25° C. for 2 h. It was then extracted with ethyl acetate (20 mL*2), the combined organic layer was washed with water (10 mL*2), dried over sodium sulfate and concentrated. The resultant crude product was purified by silica gel column chromatography (23% ethyl acetate in petroleum ether) to obtain 1-(2-chloro-9-ethyl-6-(4-hydroxypiperidin-1-yl)-9H-purin-8-yl)ethan-1-one as white solid. (350.0 mg, 77.8%). LCMS (ESI) m/z: 324.3 [M+H]⁺.

### Step 2: Synthesis of 1-(9-ethyl-6-(4-hydroxypiperidin-1-yl)-2-(3-(1-methyl-1H-pyrazol-3-yl)phenyl)-9H-purin-8-yl)ethan-1-one

To a solution of 1-(2-chloro-9-ethyl-6-(4-hydroxypiperidin-1-yl)-9H-purin-8-yl)ethan-1-one (200 mg, 0.62 mmol) in 1,4-dioxane (6 mL) and water (2 mL) were added 1-methyl-3-(3-(4,4,5,5-tetramethyl-1,3,2-dioxaborolan-2-yl)phenyl)-1H-pyrazole (211.1 mg, 0.74 mmol), [1,1′-bis(diphenylphosphino)ferrocene]dichloropalladium(II) (45.2 mg, 0.06 mmol) and potassium carbonate (256.6 mg, 1.86 mmol) at 25° C. The resultant reaction mixture was stirred at 90° C. for 2 h under nitrogen protection. The mixture was then extracted with ethyl acetate (20 mL*2), washed with water (10 mL*2), dried over sodium sulfate, and concentrated. The residue was subjected to silica gel column chromatography (63% ethyl acetate in petroleum ether) to obtain 1-(9-ethyl-6-(4-hydroxypiperidin-1-yl)-2-(3-(1-methyl-1H-pyrazol-3-yl)phenyl)-9H-purin-8-yl)ethan-1-one as white solid (150 mg, 54.5%). LCMS (ESI) m/z: 446.4 [M+H]⁺.

### Step 3: Synthesis of 1-(9-ethyl-8-(1-hydroxyethyl)-2-(3-(1-methyl-1H-pyrazol-3-yl)phenyl)-9H-purin-6-yl)piperidin-4-ol

To a solution of 1-(9-ethyl-6-(4-hydroxypiperidin-1-yl)-2-(3-(1-methyl-1H-pyrazol-3-yl)phenyl)-9H-purin-8-yl)ethan-1-one (150.0 mg, 0.34 mmol) in methanol (20 mL) was added sodium borohydride (63.7 mg, 1.68 mmol). The mixture was stirred at 0° C. for 0.5 h and at 25° C. for 2 h. It was quenched by water, the mixture was extracted with dichloromethane (20 mL*2) and washed with water (10 mL*2). The combined organic layer was dried over sodium sulfate and concentrated. The resultant crude product was purified by silica gel column chromatography (5% methanol in dichloromethane) to obtain 1-(9-ethyl-8-(1-hydroxyethyl)-2-(3-(1-methyl-1H-pyrazol-3-yl)phenyl)-9H-purin-6-yl)piperidin-4-ol as white solid (81.8 mg, 54.5%). ¹H NMR (400 MHz, DMSO-d₆) δ 8.76 (s, 1H), 8.30 (d, J=7.9 Hz, 1H), 7.83 (d, J=7.7 Hz, 1H), 7.78 (d, J=2.1 Hz, 1H), 7.49 (t, J=7.7 Hz, 1H), 6.72 (d, J=2.2 Hz, 1H), 5.70 (bs, 1H), 5.04-4.55 (m, 4H), 4.40 (q, J=6.9 Hz, 2H), 3.92 (s, 3H), 3.86-3.75 (m, 1H), 3.69 (s, 2H), 1.91 (d, J=9.4 Hz, 2H), 1.59 (d, J=6.5 Hz, 3H), 1.51-1.37 (m, 5H). LCMS (ESI) m/z: 448.4 [M+H]⁺.

### Synthesis of 4-(9-ethyl-2-(3-(1-methyl-1H-pyrazol-3-yl)phenyl)-9H-purin-6-yl)morpholine (Compound 365)

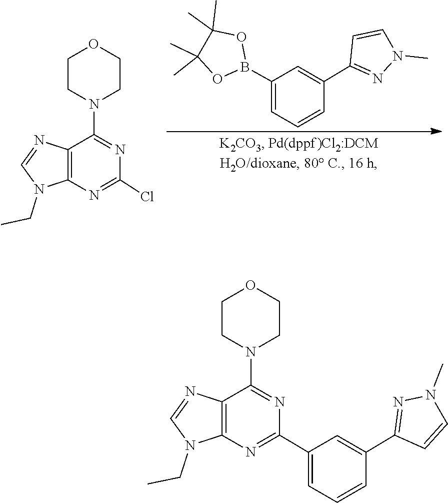

A mixture of 4-(2-chloro-9-ethyl-9H-purin-6-yl)morpholine (78 mg, 0.3 mmol), 1-methyl-3-(3-(4,4,5,5-tetramethyl-1,3,2-dioxaborolan-2-yl)phenyl)-1H-pyrazole (102 mg, 0.36 mmol), 1,1′-bis(diphenylphosphino)ferrocene-palladium(II)dichloride dichloromethane complex (51.9 mg, 0.45 mmol) and potassium carbonate (300 mg, 0.9 mmol) in 1,4-dioxane (10 mL) with water (2 mL) was stirred at 95° C. under argon atmosphere for 16 h. The reaction mixture was filtered and the filtrate was subjected to prep-HPLC (Column Xbridge 21.2*250 mm C18, 10 um, Mobile Phase A: water (10 mmol/L ammonium bicarbonate) B: acetonitrile) to obtain 4-(9-ethyl-2-(3-(1-methyl-1H-pyrazol-3-yl)phenyl)-9H-purin-6-yl)morpholine (33.5 mg, 28.6%) as white solid. ¹H NMR (400 MHz, DMSO-d₆) δ 8.76 (s, 1H), 8.31 (d, J=7.8 Hz, 1H), 8.26 (s, 1H), 7.85 (d, J=7.8 Hz, 1H), 7.78 (d, J=2.2 Hz, 1H), 7.49 (t, J=7.7 Hz, 1H), 6.74 (d, J=2.2 Hz, 1H), 4.4-4.2 (m, 6H), 3.92 (s, 3H), 3.81-3.75 (m, 4H), 1.48 (t, J=7.3 Hz, 3H). LCMS (ESI) m/z: 390.3 [M+H]⁺.

### Synthesis of 9-ethyl-2-(3-(1-methyl-1H-pyrazol-3-yl)phenyl)-6-(pyridin-4-yl)-9H-purine (Compound 366)

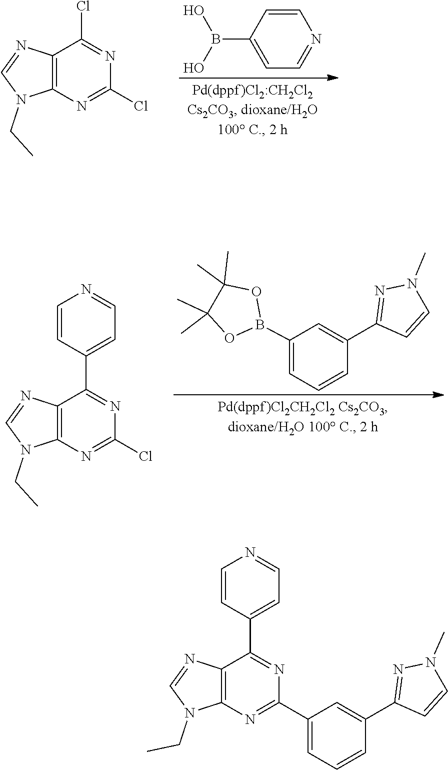 [SMILES: CCn1cnc2c(Cl)nc(Cl)nc21 ; OB(O)c1ccncc1 ; CCn1cnc2c(-c3ccncc3)nc(Cl)nc21 ; Cn1ccc(-c2cccc(B3OC(C)(C)C(C)(C)O3)c2)n1 ; CCn1cnc2c(-c3ccncc3)nc(-c3cccc(-c4ccn(C)n4)c3)nc21]

### Step 1: Synthesis of 2-chloro-9-ethyl-6-(pyridin-4-yl)-9H-purine

A mixture of 2,6-dichloro-9-ethyl-9H-purine (1.08 g, 5.0 mmol), pyridin-4-ylboronic acid (615 mg, 5.0 mmol), 1,1′-bis(diphenylphosphino)ferrocene-palladium(II)dichloride dichloromethane complex (204 mg, 0.25 mmol) and cesium carbonate (3.25 g, 10.0 mmol) in dioxane (20 mL) and water (4.0 mL) was stirred at 100° C. under nitrogen atmosphere for 2 h. The reaction mixture was poured into water and extracted with ethyl acetate (200 mL*2). The combined organic phase was concentrated and the residue was subjected to silica gel column chromatography (100% ethyl acetate) to obtain 2-chloro-9-ethyl-6-(pyridin-4-yl)-9H-purine (650 mg, 50%) as purple solid. ¹H NMR (400 MHz, CDCl₃) δ 8.84 (dd, J=4.6, 1.6 Hz, 2H), 8.63 (dd, J=4.5, 1.6 Hz, 2H), 8.19 (s, 1H), 4.38 (q, J=7.4 Hz, 2H), 1.61 (t, J=7.4 Hz, 3H); LCMS (ESI) m/z: 260.1/262.0 [M+H]⁺.

### Step 2: Synthesis of 9-ethyl-2-(3-(1-methyl-1H-pyrazol-3-yl)phenyl)-6-(pyridin-4-yl)-9H-purine

A mixture of 2-chloro-9-ethyl-6-(pyridin-4-yl)-9H-purine (259 mg, 1.0 mmol), 1-methyl-3-(3-(4,4,5,5-tetramethyl-1,3,2-dioxaborolan-2-yl)phenyl)-1H-pyrazole (284 mg, 1.0 mmol), 1,1′-bis(diphenylphosphino)ferrocene-palladium(II)dichloride dichloromethane complex (41 mg, 0.05 mmol) and cesium carbonate (650 mg, 2.0 mmol) in dioxane (8 mL) and water (0.5 mL) was stirred at 100° C. under nitrogen atmosphere for 2 h. The reaction mixture was then poured into water and extracted with ethyl acetate (100 mL*2). The combined organic phase was concentrated and the residue was subjected to silica gel column chromatography (10% methanol in dichloromethane) followed by prep-HPLC (Column Xbridge 21.2*250 mm C18, 10 um, Mobile Phase A: water (10 mmol/L ammonium bicarbonate) B: acetonitrile) to obtain 9-ethyl-2-(3-(1-methyl-1H-pyrazol-3-yl)phenyl)-6-(pyridin-4-yl)-5,9-dihydro-4H-purine (56.7 mg, 15%) as white solid. ¹H NMR (400 MHz, CDCl3) δ 9.04 (d, J=1.5 Hz, 1H), 8.87 (dd, J=4.6, 1.5 Hz, 2H), 8.81 (dd, J=4.6, 1.5 Hz, 2H), 8.61 (d, J=7.9 Hz, 1H), 8.19 (s, 1H), 7.96 (d, J=7.7 Hz, 1H), 7.58 (t, J=7.7 Hz, 1H), 7.45 (d, J=2.2 Hz, 1H), 6.71 (d, J=2.2 Hz, 1H), 4.48 (q, J=7.3 Hz, 2H), 4.01 (s, 3H), 1.67 (t, J=7.3 Hz, 3H); LCMS (ESI) m/z: 381.8 [M]⁺.

### Synthesis of 4-(9-ethyl-2-(3-(1-methyl-1H-pyrazol-3-yl)phenyl)-6-(piperidin-1-yl)-9H-purin-8-yl)-1-methylpiperazin-2-one (Compound 367)

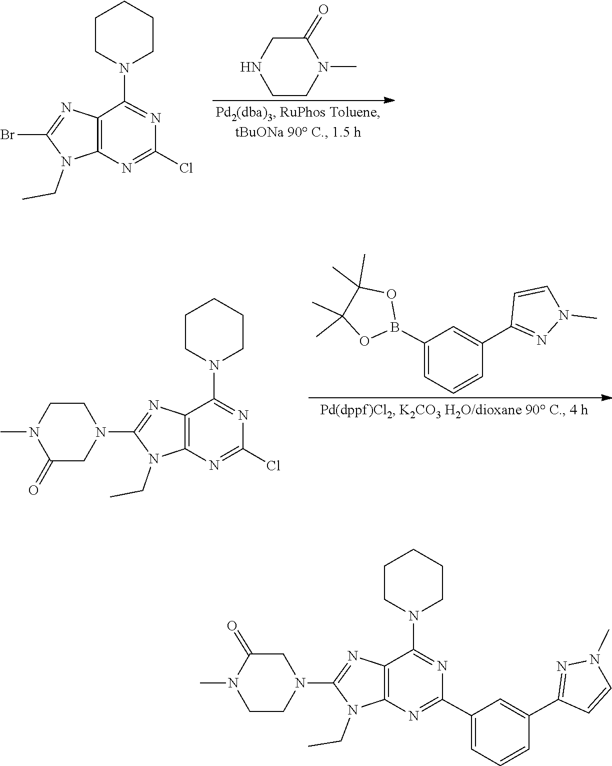

### Step 1: Synthesis of 4-(2-chloro-9-ethyl-6-(piperidin-1-yl)-9H-purin-8-yl)-1-methylpiperazin-2-one

A mixture of 8-bromo-2-chloro-9-ethyl-6-(piperidin-1-yl)-9H-purine (100 mg, 0.290 mmol), 1-methylpiperazin-2-one (33 mg, 0.290 mmol), tris(dibenzylideneacetone)dipalladium (30 mg, 0.05 mmol), 2-dicyclohexylphosphino-2′,6′-diisopropoxy-1,1′-biphenyl (56 mg, 0.06 mmol) and sodium tert-butoxide (60 mg, 0.620 mmol) in toluene (10 mL) was stirred at 85° C. for 16 h. Then the reaction was quenched with water (15 mL) and the mixture was extracted with ethyl acetate (20 mL*3). The combined organic layer was washed with brine (30 mL), dried over sodium sulfate, filtered and concentrated. The resultant residue was subjected to silica gel column chromatography (petroleum ether:ethyl acetate=75:25) to obtain 4-(2-chloro-9-ethyl-6-(piperidin-1-yl)-9H-purin-8-yl)-1-methylpiperazin-2-one (50 mg, 45.6%) as yellow solid. LCMS (ESI) m/z: 377.9[M]⁺

### Step 2: Synthesis of 4-(9-ethyl-2-(3-(1-methyl-1H-pyrazol-3-yl)phenyl)-6-(piperidin-1-yl)-9H-purin-8-yl)-1-methylpiperazin-2-one

To a solution of 4-(2-chloro-9-ethyl-6-(piperidin-1-yl)-9H-purin-8-yl)-1-methylpiperazin-2-one (50 mg, 0.133 mmol) in 1,4-dioxane (5 mL) and water (5 mL) were added 1-methyl-3-(3-(4,4,5,5-tetramethyl-1,3,2-dioxaborolan-2-yl)phenyl)-1H-pyrazole (95 mg, 0.332 mmol,), potassium carbonate (37 mg, 0.266 mmol) and dichloro[1,1′-bis(diphenylphosphino)ferrocene]palladium(II) dichloromethane adduct (50 mg, 0.196 mmol). The mixture was stirred at 90° C. for 2 h and concentrated. Water (20 mL) was added to the residue, and it was extracted with ethyl acetate (15 mL×2). The combined organic layer was washed with brine (30 mL), dried over sodium sulfate, filtered and concentrated. The residue was subjected to prep-HPLC (0.05% ammonium bicarbonate:acetonitrile=5%-95%) to obtain 4-(9-ethyl-2-(3-(1-methyl-1H-pyrazol-3-yl)phenyl)-6-(piperidin-1-yl)-9H-purin-8-yl)-1-methylpiperazin-2-one (10.3 mg, 16%) as white solid. ¹H NMR (400 MHz, DMSO-d₆) δ 8.73 (s, 1H), 8.27 (d, J=7.9 Hz, 1H), 7.79 (dd, J=16.4, 4.9 Hz, 2H), 7.48 (t, J=7.7 Hz, 1H), 6.71 (d, J=2.2 Hz, 1H), 4.42-4.06 (m, 6H), 3.92 (s, 3H), 3.88 (s, 2H), 3.55-3.48 (m, 4H), 2.91 (s, 3H), 1.72-1.51 (m, 6H), 1.44 (t, J=7.1 Hz, 3H). LCMS (ESI) m/z: 499.8[M]⁺.

### Synthesis of 2-(9-ethyl-2-(3-(1-methyl-1H-pyrazol-3-yl)phenyl)-6-morpholino-9H-purin-8-yl)propan-2-ol (Compound 368)

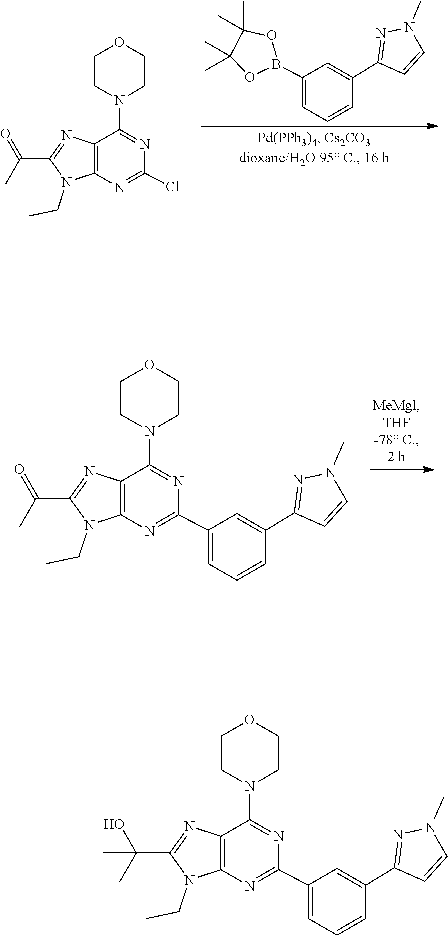

### Step 1: Synthesis of 1-(9-ethyl-2-(3-(1-methyl-1H-pyrazol-3-yl)phenyl)-6-morpholino-9H-purin-8-yl)ethenone

A solution of 1-(2-chloro-9-ethyl-6-morpholino-9H-purin-8-yl)ethanone (93 mg, 0.3 mmol), 1-methyl-3-(3-(4,4,5,5-tetramethyl-1,3,2-dioxaborolan-2-yl)phenyl)-1H-pyrazole (128 mg, 0.45 mmol), tetrakis(triphenylphosphine) palladium (52 mg, 0.045 mmol) and cesium carbonate (391 mg, 1.2 mmol) in water (1 mL) and dioxane (10 mL) was stirred at 95° C. for 16 h under argon atmosphere. The resultant reaction mixture was cooled and concentrated. The residue was subjected to silica gel column chromatography (methanol/dichloromethane=0%-3%) to obtain 1-(9-ethyl-2-(3-(1-methyl-1H-pyrazol-3-yl)phenyl)-6-morpholino-9H-purin-8-yl)ethanone (230 mg) as yellow solid. LCMS: (ESI) m/z: 432.1 [M+H]⁺.

### Step 2: Synthesis of 2-(9-ethyl-2-(3-(1-methyl-1H-pyrazol-3-yl)phenyl)-6-morpholino-9H-purin-8-yl)propan-2-ol

Methyl magnesium iodide (1 M solution in tetrahydrofuran, 1 mL, 1 mmol) was added into a solution of 1-(9-ethyl-2-(3-(1-methyl-1H-pyrazol-3-yl)phenyl)-6-morpholino-9H-purin-8-yl)ethanone (210 mg, 0.49 mmol) in tetrahydrofuran (10 mL) at −78° C. and the mixture was stirred for 2 h at that temperature. Water (10 mL) was added to above mixture and it was extracted with dichloromethane (100 mL). The organic phase was washed with water (50 mL), dried over sodium sulfate and concentrated. The residue was subjected to prep-HPLC (BOSTON pHlex ODS 10 μm 21.2×250 mm 120 A. The mobile phase was acetonitrile/0.1% ammonium bicarbonate) to obtain 2-(9-ethyl-2-(3-(1-methyl-1H-pyrazol-3-yl)phenyl)-6-morpholino-9H-purin-8-yl)propan-2-ol (32.9 mg, 18% for two steps) as white solid. ¹H NMR (400 MHz, DMSO-d₆) δ 8.75 (t, J=1.5 Hz, 1H), 8.32-8.29 (m, 1H), 7.85-7.83 (m, 1H), 7.78 (d, J=2.2 Hz, 1H), 7.49 (t, J=7.7 Hz, 1H), 6.73 (d, J=2.2 Hz, 1H), 5.69 (s, 1H), 4.59 (q, J=6.7 Hz, 2H), 4.30 (bs, 4H), 3.92 (s, 3H), 3.77 (t, J=4.4 Hz, 4H), 1.63 (s, 6H), 1.47 (t, J=7.0 Hz, 3H). LCMS: (ESI) m/z: 448.2 [M+H]⁺.

### Synthesis of 8-(9-ethyl-2-(3-(1-methyl-1H-pyrazol-3-yl)phenyl)-6-morpholino-9H-purin-8-yl)octahydropyrazino[2,1-c][1,4]oxazine (Compound 369)

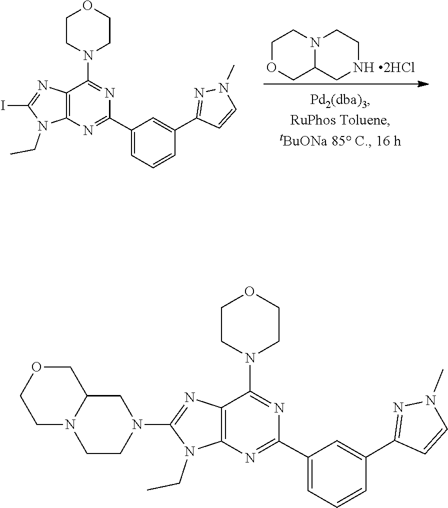

A solution of (4-(9-ethyl-8-iodo-2-(3-(1-methyl-1H-pyrazol-3-yl)phenyl)-9H-purin-6-yl)morpholine (77 mg, 0.15 mmol), octahydropyrazino[2,1-c][1,4]oxazine dihydrochloride (32 mg, 0.15 mmol), tris(dibenzylideneacetone)dipalladium (27 mg, 0.03 mmol), 2-dicyclohexylphosphino-2′,6′-diisopropoxybiphenyl (28 mg, 0.06 mmol), and sodium tert-butoxide (86 mg, 0.9 mmol) in toluene (10 mL) was stirred at 85° C. for 16 h under argon atmosphere. The reaction mixture was cooled, filtered and concentrated. The residue was subjected to prep-HPLC (BOSTON pHlex ODS 10 μm 21.2×250 mm 120 A. The mobile phase was acetonitrile/0.1% ammonium bicarbonate) to obtain 8-(9-ethyl-2-(3-(1-methyl-1H-pyrazol-3-yl)phenyl)-6-morpholino-9H-purin-8-yl) octahydropyrazino[2,1-c][1,4]oxazine (29.1 mg, 27%) as white solid. ¹H NMR (400 MHz, DMSO-d₆) δ 8.72 (t, J=1.5 Hz, 1H), 8.28 (dt, J=7.5, 1.2 Hz, 1H), 7.82 (dt, J=7.6, 1.6 Hz, 1H), 7.77 (d, J=2.2 Hz, 1H), 7.47 (t, J=7.7 Hz, 1H), 6.73 (d, J=2.2 Hz, 1H), 4.28-4.13 (m, 6H), 3.92 (s, 3H), 3.77 (t, J=4.5 Hz, 6H), 3.59-3.47 (m, 2H), 3.17 (t, J=10.2 Hz, 1H), 3.05 (t, J=10.9 Hz, 1H), 2.83 (d, J=10.0 Hz, 1H), 2.73-2.61 (m, 2H), 2.49-2.23 (m, 4H), 1.44 (t, J=7.2 Hz, 3H). LCMS (ESI) m/z: 530.3 [M+H]⁺.

### Synthesis of 8-(9-ethyl-6-((R)-3-methylmorpholino)-2-(4-phenyl-1H-pyrazol-1-yl)-9H-purin-8-yl)octahydropyrazino[2,1-c][1,4]oxazine (Compound 370)

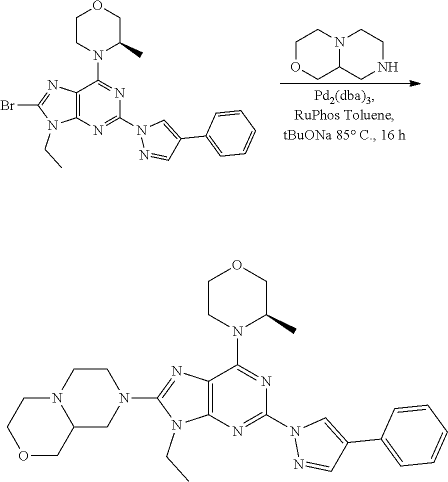 [SMILES: CCn1c(Br)nc2c(N3CCOC[C@H]3C)nc(-n3cc(-c4ccccc4)cn3)nc21 ; C1CN2CCOCC2CN1 ; CCn1c(N2CCN3CCOCC3C2)nc2c(N3CCOC[C@H]3C)nc(-n3cc(-c4ccccc4)cn3)nc21]

The compound 32 was synthesized according to the protocol described above for the compound 31.

1H NMR (400 MHz, DMSO-d₆) δ 8.99 (s, 1H), 8.21 (s, 1H), 7.77 (d, J=7.3 Hz, 2H), 7.41 (t, J=7.7 Hz, 2H), 7.27 (t, J=7.3 Hz, 1H), 5.37 (s, 1H), 5.07 (s, 1H), 4.13 (d, J=6.9 Hz, 2H), 3.99 (d, J=9.2 Hz, 1H), 3.82-3.68 (m, 4H), 3.55 (t, J=11.5 Hz, 2H), 3.48 (d, J=13.6 Hz, 3H), 3.18-3.01 (m, 2H), 2.82 (d, J=10.9 Hz, 1H), 2.71-2.62 (m, 2H), 2.42 (s, 1H), 2.33 (s, 1H), 2.28 (s, 1H), 1.40 (t, J=7.2 Hz, 3H), 1.32 (d, J=6.7 Hz, 3H). LCMS (ESI) m/z: 530.3 [M+H]⁺.

### Synthesis of 2-[[9-ethyl-6-morpholino-8-(4-pyridyl)purin-2-yl]-propyl-amino]-1-(3-pyridyl)ethanol (Compound 371)

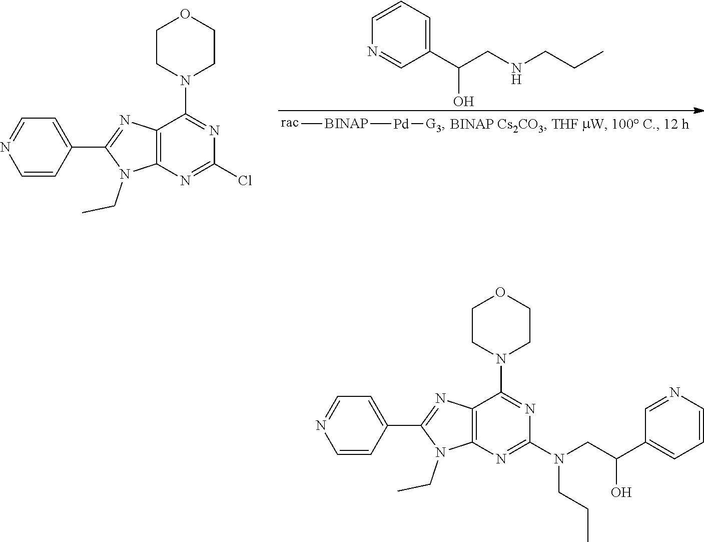

To a solution of 4-[2-chloro-9-ethyl-8-(4-pyridyl)purin-6-yl]morpholine (100 mg, 290 umol) in THE (3 mL) were added 2-(propylamino)-1-(3-pyridyl)ethanol (63 mg, 348 umol), Cs₂CO₃ (283 mg, 870 umol), BINAP (18 mg, 29 umol) and rac-BINAP-Pd-G3 (29 mg, 29 umol). The resulting mixture was stirred at 100° C. for 12 h under microwave irradiation. The reaction mixture was filtered and the filtrate was subjected to prep-HPLC (Phenomenex Luna C18 150*30 mm*5 um column; 10-40% acetonitrile in an a 0.2% formic acid solution in water, 8 min gradient) to obtain 2-[[9-ethyl-6-morpholino-8-(4-pyridyl)purin-2-yl]-propyl-amino]-1-(3-pyridyl)ethanol (15 mg, 10%) as white solid. ¹H NMR (400 MHz, CHLOROFORM-d) δ 8.82-8.64 (m, 3H), 8.57 (d, J=4.2 Hz, 1H), 7.87 (d, J=7.5 Hz, 1H), 7.66 (d, J=5.7 Hz, 2H), 7.48-7.33 (m, 1H), 7.21 (bs, 1H), 5.14 (d, J=6.8 Hz, 1H), 4.48-4.09 (m, 5H), 3.97 (dd, J=14.9, 7.5 Hz, 1H), 3.90-3.70 (m, 5H), 3.63-3.46 (m, 1H), 3.25-3.07 (m, 1H), 1.65-1.55 (m, 2H), 1.51 (t, J=7.2 Hz, 3H), 0.87 (t, J=7.6 Hz, 3H). LCMS (ESI for C26H32N8O2) [M+H]⁺: 489.2.

### Synthesis of 4-(9-ethyl-6-morpholino-2-(3-(pyridazin-3-yl)phenyl)-9H-purin-8-yl)-1-methylpiperazin-2-one (Compound 372)

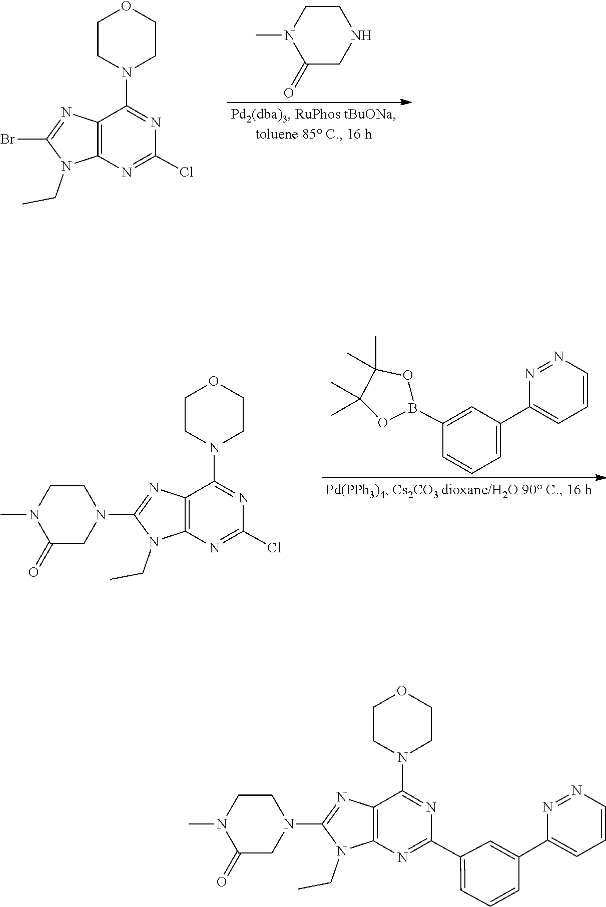

### Step 1: Synthesis of 4-(2-chloro-9-ethyl-6-morpholino-9H-purin-8-yl)-1-methylpiperazin-2-one

A solution of 4-(8-bromo-2-chloro-9-ethyl-9H-purin-6-yl)morpholine (100 mg, 0.29 mmol), 1-methylpiperazin-2-one (33 mg, 0.29 mmol), tris(dibenzylideneacetone)dipalladium (27.5 mg, 0.03 mmol), 2-dicyclohexylphosphino-2′,6′-diisopropoxybiphenyl (27.5 mg, 0.06 mmol), and sodium tert-butoxide (115 mg, 1.2 mmol) in dry toluene (6 mL) was stirred at 85° C. for 16 h under argon atmosphere. The reaction mixture was concentrated and subjected to prep-HPLC (BOSTON pHlex ODS 1 0 um 21.2×250 mm 120 A. The mobile phase was acetonitrile/0.1% ammonium bicarbonate) to obtain 4-(2-chloro-9-ethyl-6-morpholino-9H-purin-8-yl)-1-methylpiperazin-2-one (37 mg, 33.45%) as white solid. LCMS (ESI) m/z: 379.8 [M+H]⁺.

### Step 2: Synthesis of 4-(9-ethyl-6-morpholino-2-(3-(pyridazin-3-yl)phenyl)-9H-purin-8-yl)-1-methylpiperazin-2-one

A solution of 4-(2-chloro-9-ethyl-6-morpholino-9H-purin-8-yl)-1-methylpiperazin-2-one (27 mg, 0.071 mmol), 3-(3-(4,4,5,5-tetramethyl-1,3,2-dioxaborolan-2-yl)phenyl)pyridazine (30 mg, 0.106 mmol), cesium carbonate (60 mg, 0.184 mmol) and tetrakis(triphenylphosphine)palladium (8 mg, 0.01 mmol) in dioxane/water (5 mL/1 mL) was stirred at 90° C. for 16 h. The resultant mixture was concentrated and subjected to prep-HPLC (BOSTON pHlex ODS 10 um 21.2×250 mm 120 A. The mobile phase was acetonitrile/0.1% ammonium bicarbonate) to obtain 4-(9-ethyl-6-morpholino-2-(3-(pyridazin-3-yl)phenyl)-9H-purin-8-yl)-1-methylpiperazin-2-one (11.1 mg, 22.79%) as white solid. ¹H NMR (400 MHz, DMSO-d₆) δ 9.26 (dd, J=4.9, 1.5 Hz, 1H), 9.11 (s, 1H), 8.54 (d, J=8.0 Hz, 1H), 8.29 (dd, J=8.7, 1.5 Hz, 1H), 8.17 (d, J=8.1 Hz, 1H), 7.84 (dd, J=8.6, 4.9 Hz, 1H), 7.67 (t, J=7.8 Hz, 1H), 4.28-4.19 (m, 6H), 3.90 (s, 2H), 3.80-3.75 (m, 4H), 3.60-3.54 (m, 2H), 3.49 (t, J=5.2 Hz, 2H), 2.91 (s, 3H), 1.45 (t, J=7.2 Hz, 3H). LCMS (ESI) m/z: 499.8 [M]⁺.

### Synthesis of 4-(9-ethyl-2-(3-methoxy-4-phenyl-1H-pyrazol-1-yl)-6-morpholino-9H-purin-8-yl)-1-methylpiperidin-2-one (Compound 373)

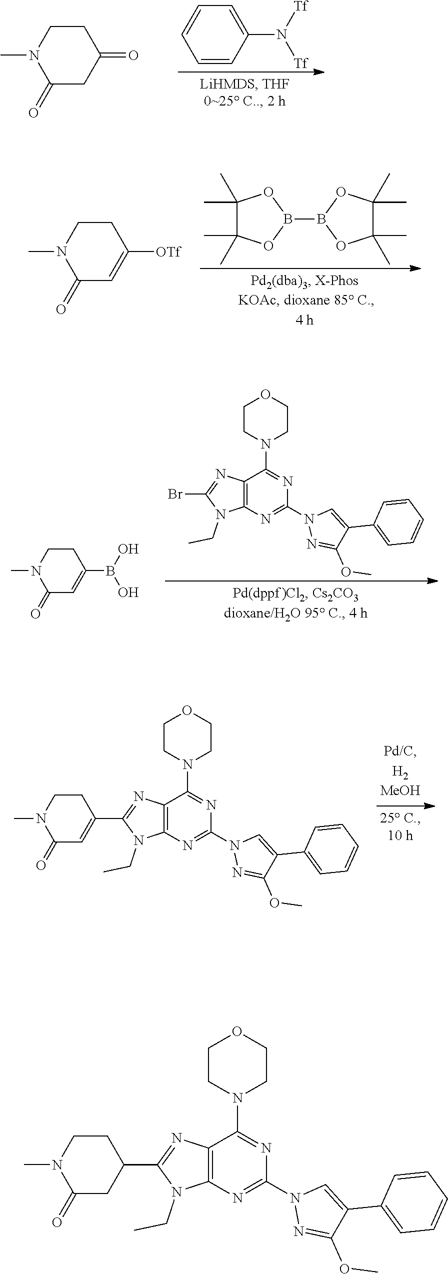

### Step 1: Synthesis of 1-methyl-6-oxo-1,2,3,6-tetrahydropyridin-4-yl trifluoromethanesulfonate

To a solution of 1-methylpiperidine-2,4-dione (0.6 g, 4.7 mmol) in tetrahydrofuran (15 mL) was added lithium bis(trimethylsilyl)amide (5.4 mL, 5.4 mmol) at −70° C. slowly. The mixture was stirred at −70° C. for 0.5 h followed by the addition of a solution of 1,1,1-trifluoro-N-phenyl-N-((trifluoromethyl)sulfonyl)methanesulfonamide (1.9 g, 5.4 mmol) in tetrahydrofuran (10 mL) at the same temperature. The mixture was then warmed up and stirred at 25° C. for 4 h. It was concentrated and the residue was dissolved in ethyl acetate (50 mL), washed with aqueous ammonium chloride (20 mL), dried over sodium sulfate and concentrated. The residue was subjected to flash chromatography (petroleum ether/ethyl acetate=3:1) to obtain 1-methyl-6-oxo-1,2,3,6-tetrahydropyridin-4-yl trifluoromethanesulfonate (0.7 g, 57%) as light yellow oil. LCMS (ESI) m/z: 260.1 [M+H]⁺.

### Step 2: Synthesis of (1-methyl-6-oxo-1,2,3,6-tetrahydropyridin-4-yl)boronic acid

A mixture of 1-methyl-6-oxo-1,2,3,6-tetrahydropyridin-4-yl trifluoromethanesulfonate (0.3 g, 1.16 mmol), 4,4,4′,4′,5,5,5′,5′-octamethyl-2,2′-bi(1,3,2-dioxaborolane (0.38 g, 1.5 mmol), potassium acetate (0.25 g, 2.55 mmol), tris(dibenzylideneacetone) dipalladium (106 mg, 0.12 mmol), and 2-(dicyclohexylphosphino)-2′,4′,6′-triisopropylbiphenyl (0.086 g, 0.18 mmol) in 1,4-dioxane (10 mL) was stirred at 85° C. for 4 h. The reaction mixture was cooled down and used directly in next step without further purification. LCMS (ESI) m/z: 156.1 [M+H]⁺.

### Step 3: Synthesis of 4-(9-ethyl-2-(3-methoxy-4-phenyl-1H-pyrazol-1-yl)-6-morpholino-9H-purin-8-yl)-1-methyl-5,6-dihydropyridin-2(1H)-one

To a solution of (1-methyl-6-oxo-1,2,3,6-tetrahydropyridin-4-yl)boronic acid (0.09 g, 0.58 mmol) in 1,4-dioxane (5 mL, from the above step) were added 4-(8-bromo-9-ethyl-2-(3-methoxy-4-phenyl-1H-pyrazol-1-yl)-9H-purin-6-yl)morpholine (0.25 g, 0.52 mmol), cesium carbonate (0.42 g, 1.29 mmol), [1,1′-bis(diphenylphosphino)ferrocene]dichloropalladium(II) (0.037 g, 0.05 mmol) and water (1.5 mL) at room temperature. The resultant reaction mixture was stirred at 95° C. for 4 h under nitrogen atmosphere. It was then filtered to remove the solids and the filtrate was concentrated. The residue was then subjected to flash chromatography (dichloromethane:methanol=15:1) to obtain 4-(9-ethyl-2-(3-methoxy-4-phenyl-1H-pyrazol-1-yl)-6-morpholino-9H-purin-8-yl)-1-methyl-5,6-dihydropyridin-2(1H)-one as yellow solid (0.15 g, 56%). LCMS (ESI) m/z: 515.1 [M+H]⁺.

### Step 4: Synthesis of 4-(9-ethyl-2-(3-methoxy-4-phenyl-1H-pyrazol-1-yl)-6-morpholino-9H-purin-8-yl)-1-methylpiperidin-2-one

A suspension of 4-(9-ethyl-2-(3-methoxy-4-phenyl-1H-pyrazol-1-yl)-6-morpholino-9H-purin-8-yl)-1-methyl-5,6-dihydropyridin-2(1H)-one (150 mg, 0.29 mmol) and palladium on activated carbon 10% (60 mg) in methanol (10 mL) was stirred at 25° C. for 10 h under hydrogen atmosphere. It was filtered to remove the solids and the filtrate was concentrated. The resultant residue was subjected to prep-HPLC (BOSTON pHlex ODS 10 μm 21.2×250 mm 120 A, with mobile phase acetonitrile/0.1% ammonium bicarbonate) to obtain 4-(9-ethyl-2-(3-methoxy-4-phenyl-1H-pyrazol-1-yl)-6-morpholino-9H-purin-8-yl)-1-methylpiperidin-2-one (26 mg, 17%) as white solid. ¹H NMR (400 MHz, DMSO-d₆) δ 8.91 (s, 1H), 7.85-7.76 (m, 2H), 7.39 (t, J=7.7 Hz, 2H), 7.24 (t, J=7.4 Hz, 1H), 4.30 (bs, 4H), 4.25 (q, J=7.2 Hz, 6H), 4.07 (s, 3H), 3.80-3.71 (m, 4H), 3.60 (d, J=9.3 Hz, 1H), 3.44 (d, J=9.8 Hz, 1H), 3.29-3.23 (m, 1H), 2.85 (s, 3H), 2.60 (t, J=6.8 Hz, 2H), 2.14 (bs, 1H), 1.98 (bs, 1H), 1.38 (t, J=7.2 Hz, 3H). LCMS (ESI) m/z: 516.8 [M]⁺.

### Preparation of enantiomer 1 (Compound 374) and enantiomer 2 (Compound 375) of 4-(9-ethyl-2-(3-methoxy-4-phenyl-1H-pyrazol-1-yl)-6-morpholino-9H-purin-8-yl)-1-methylpiperidin-2-one

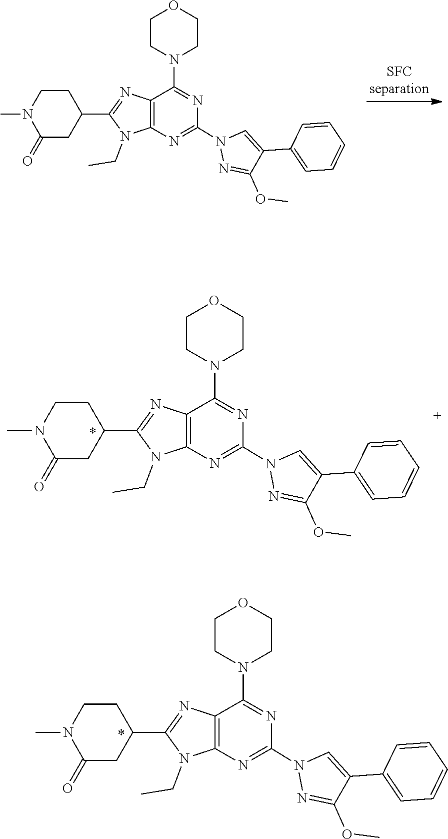 [SMILES: CCn1c(C2CCN(C)C(=O)C2)nc2c(N3CCOCC3)nc(-n3cc(-c4ccccc4)c(OC)n3)nc21 ; CCn1c(C2CCN(C)C(=O)C2)nc2c(N3CCOCC3)nc(-n3cc(-c4ccccc4)c(OC)n3)nc21 ; CCn1c(C2CCN(C)C(=O)C2)nc2c(N3CCOCC3)nc(-n3cc(-c4ccccc4)c(OC)n3)nc21]

The racemic product was subjected to chiral prep-HPLC conditions (Instrument: SFC-80 (Thar, Waters), Column: AD 20*250 mm, 10 um (Daicel), Column temperature: 35° C., Mobile phase: carbon dioxide/ethanol (0.5% methanol ammonia)=65/35, Flow rate: 80 g/min, Back pressure: 100 bar, Detection wavelength: 214 nm, Cycle time: 3.0 min, Sample solution: 65 mg dissolved in 15 ml methanol, Injection volume: 1.0 ml) to obtain the two enantiomers: enantiomer 1 (27.6 mg, 42%) and enantiomer 2 (27.5 mg, 42%) as off-white solids.

Compound 36: ¹H NMR (400 MHz, DMSO-d₆) δ 8.91 (s, 1H), 7.85-7.76 (m, 2H), 7.39 (t, J=7.7 Hz, 2H), 7.24 (t, J=7.4 Hz, 1H), 4.55-4.05 (m, 6H), 4.07 (s, 3H), 3.80-3.71 (m, 4H), 3.60 (d, J=9.3 Hz, 1H), 3.44 (d, J=9.8 Hz, 1H), 3.29-3.23 (m, 1H), 2.85 (s, 3H), 2.60 (t, J=6.8 Hz, 2H), 2.14 (s, 1H), 1.98 (s, 1H), 1.38 (t, J=7.2 Hz, 3H). LCMS (ESI) m/z: 517.2 [M+H]⁺; (RT: 2.18 min.)

Compound 37: ¹H NMR (400 MHz, DMSO-d₆) δ 8.91 (s, 1H), 7.85-7.76 (m, 2H), 7.39 (t, J=7.7 Hz, 2H), 7.24 (t, J=7.4 Hz, 1H), 4.50-4.05 (m, 6H), 4.07 (s, 3H), 3.80-3.71 (m, 4H), 3.60 (d, J=9.3 Hz, 1H), 3.44 (d, J=9.8 Hz, 1H), 3.29-3.23 (m, 1H), 2.85 (s, 3H), 2.60 (t, J=6.8 Hz, 2H), 2.14 (s, 1H), 1.98 (s, 1H), 1.38 (t, J=7.2 Hz, 3H). LCMS (ESI) m/z: 517.2 [M+H]⁺; (RT: 2.61 min).

### Preparation of (4-(9-ethyl-2-(3-(1-methyl-1H-pyrazol-3-yl)phenyl)-8-(pyridin-4-yl)-9H-purin-6-yl)morpholin-3-yl)methanol (Compound 376, enantiomer 1) and (4-(9-ethyl-2-(3-(1-methyl-1H-pyrazol-3-yl)phenyl)-8-(pyridin-4-yl)-9H-purin-6-yl)morpholin-3-yl)methanol (Compound 377, enantiomer 2)

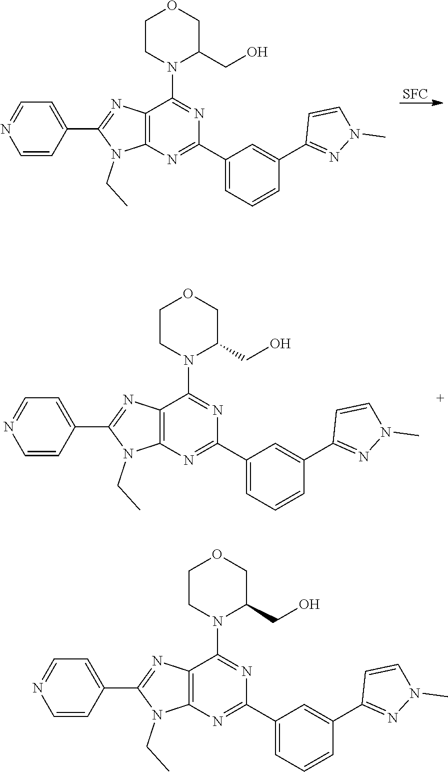

The racemic compound (4-(9-ethyl-2-(3-(1-methyl-1H-pyrazol-3-yl)phenyl)-8-(pyridin-4-yl)-9H-purin-6-yl)morpholin-3-yl)methanol (100 mg, 0.2 mmol) was subjected to chiral prep-HPLC (Instrument: SFC-150 (Waters), Column: AS 20*250 mm, 10 um (Daicel), Column temperature: 35° C. Mobile phase: carbon dioxide/methanol (0.2% methanol ammonia)=60/40, Flow rate: 100 g/min, Back pressure: 100 bar, Detection wavelength: 214 nm, Cycle time: 4 min) to afford the two enantiomers: Enantiomer 1 (4-(9-ethyl-2-(3-(1-methyl-1H-pyrazol-3-yl)phenyl)-8-(pyridin-4-yl)-9H-purin-6-yl)morpholin-3-yl)methanol (36.7 mg, 36.7%) and enantiomer 2 (4-(9-ethyl-2-(3-(1-methyl-1H-pyrazol-3-yl)phenyl)-8-(pyridin-4-yl)-9H-purin-6-yl)morpholin-3-yl)methanol (27.9 mg, 27.9%) were isolated as white solids.

Compound 38: ¹H NMR (500 MHz, DMSO-d₆) δ 8.80 (s, 3H), 8.35 (d, J=7.8 Hz, 1H), 7.88 (d, J=5.9 Hz, 3H), 7.78 (d, J=2.1 Hz, 1H), 7.52 (t, J=7.7 Hz, 1H), 6.76 (d, J=2.2 Hz, 1H), 5.75-5.60 (m, 1H), 4.94 (bs, 2H), 4.51 (q, J=7.2 Hz, 2H), 4.13 (s, 1H), 4.02 (d, J=8.9 Hz, 1H), 3.95-3.83 (m, 4H), 3.67 (d, J=8.9 Hz, 2H), 3.60 (t, J=10.5 Hz, 2H), 1.40 (t, J=7.2 Hz, 3H). LCMS (ESI) m/z: 497.1 [M+H]⁺. (RT=2.76 min.).

Compound 39: ¹H NMR (500 MHz, DMSO-d₆) δ 8.81 (d, J=6.0 Hz, 3H), 8.35 (d, J=7.8 Hz, 1H), 7.88 (d, J=7.1 Hz, 3H), 7.78 (d, J=2.1 Hz, 1H), 7.52 (t, J=7.7 Hz, 1H), 6.76 (d, J=2.2 Hz, 1H), 5.80-5.40 (m, 1H), 4.96 (bs, 2H), 4.51 (q, J=7.3 Hz, 2H), 4.13 (s, 1H), 4.02 (d, J=8.7 Hz, 1H), 3.91 (d, J=14.5 Hz, 4H), 3.67 (d, J=8.7 Hz, 2H), 3.59 (t, J=10.5 Hz, 2H), 1.40 (t, J=7.2 Hz, 3H). LCMS (ESI) m/z: 497.1 [M+H]⁺. (RT=3.28 min.).

### Synthesis of 2-(9-ethyl-2-(3-methoxy-4-phenyl-1H-pyrazol-1-yl)-6-(piperidin-1-yl)-9H-purin-8-yl)ethan-1-ol (Compound 378)

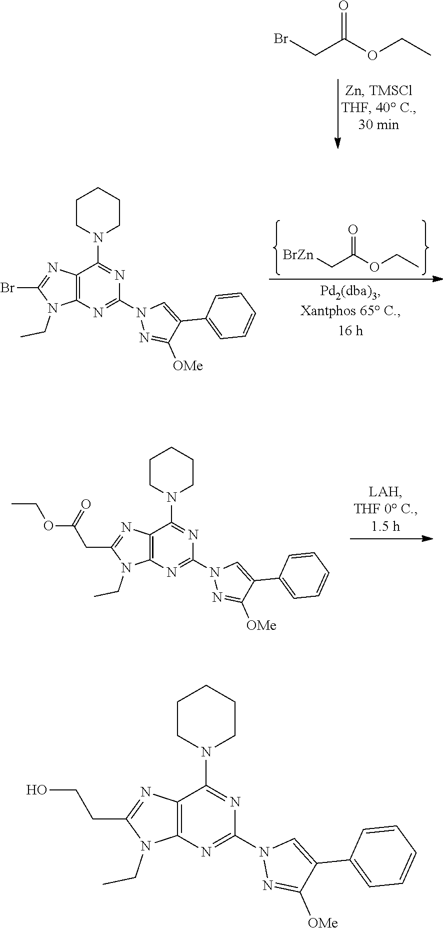

### Step 1: Synthesis of ethyl 2-(9-ethyl-2-(3-methoxy-4-phenyl-1H-pyrazol-1-yl)-6-(piperidin-1-yl)-9H-purin-8-yl)acetate

Under nitrogen atmosphere, zinc powder (2.56 g, 39.15 mmol) was suspended in tetrahydrofuran (20 mL) and trimethylsilyl chloride (0.25 mL, 2.9 mmol) was added to it at room temperature. The resultant mixture was stirred for 30 min at that temperature and then warmed up to 40° C. followed by the drop-wise addition of ethyl 2-bromoacetate (2.2 mL, 19.65 mmol). The mixture was then stirred at 40° C. for 30 min and the insoluble materials were removed by decanting to obtain a light-yellow supernatant solution. This solution was then added to a mixture of 8-bromo-9-ethyl-2-(3-methoxy-4-phenyl-1H-pyrazol-1-yl)-6-(piperidin-1-yl)-9H-purine (0.4 g, 0.83 mmol), tris(dibenzylideneacetone)dipalladium (76 mg, 0.08 mmol) and 4,5-bis(diphenylphosphino)-9,9-dimethylxanthene (96 mg, 0.16 mmol) in tetrahydrofuran (20 mL) at room temperature under argon atmosphere. After the addition, the reaction mixture was heated to 65° C. and stirred for 16 h. It was cooled down, quenched with aqueous ammonium chloride solution and extracted with ethyl acetate (20 mL×2). The combined organic phase was washed with brine (20 mL), dried over sodium sulfate, filtered and concentrated. The residue was subjected to flash chromatography (eluted with ethyl acetate in petroleum ether from 20% to 40%) to obtain ethyl 2-(9-ethyl-2-(3-methoxy-4-phenyl-1H-pyrazol-1-yl)-6-(piperidin-1-yl)-9H-purin-8-yl)acetate (0.35 g, 85.9%) as yellow solid. LCMS (ESI) m/z: 489.8 [M]⁺.

### Step 2: Synthesis of 2-(9-ethyl-2-(3-methoxy-4-phenyl-1H-pyrazol-1-yl)-6-(piperidin-1-yl)-9H-purin-8-yl)ethan-1-ol

To a solution of ethyl 2-(9-ethyl-2-(3-methoxy-4-phenyl-1H-pyrazol-1-yl)-6-(piperidin-1-yl)-9H-purin-8-yl)acetate (0.15 g, 0.307 mmol) in tetrahydrofuran (15 mL) at 0° C., was added a 1M lithium aluminum hydride solution in tetrahydrofuran (0.6 mL, 0.6 mmol) drop-wise under nitrogen atmosphere. After the addition, the reaction mixture was stirred at the same temperature for 1.5 h, then quenched with sodium sulfate decahydrate solution and the resultant solids were removed by filtration. The filtrate was concentrated and the residue was subjected to prep-HPLC (base) to obtain 2-(9-ethyl-2-(3-methoxy-4-phenyl-1H-pyrazol-1-yl)-6-(piperidin-1-yl)-9H-purin-8-yl)ethan-1-ol (30 mg, 21.8%) as white solid. ¹H NMR (500 MHz, DMSO-d₆) δ 8.87 (s, 1H), 7.80 (d, J=7.3 Hz, 2H), 7.39 (t, J=7.7 Hz, 2H), 7.24 (t, J=7.4 Hz, 1H), 4.85 (t, J=5.4 Hz, 1H), 4.40-4.22 (m, 6H), 4.07 (s, 3H), 3.83 (q, J=6.5 Hz, 2H), 3.00 (t, J=6.6 Hz, 2H), 1.86-1.46 (m, 6H), 1.36 (t, J=7.2 Hz, 3H). LCMS (ESI) m/z: 447.8 [M]⁺.

### Synthesis of 3-(9-ethyl-2-(3-methoxy-4-phenyl-1H-pyrazol-1-yl)-6-morpholino-9H-purin-8-yl)-N,N-dimethylpropanamide (Compound 379)

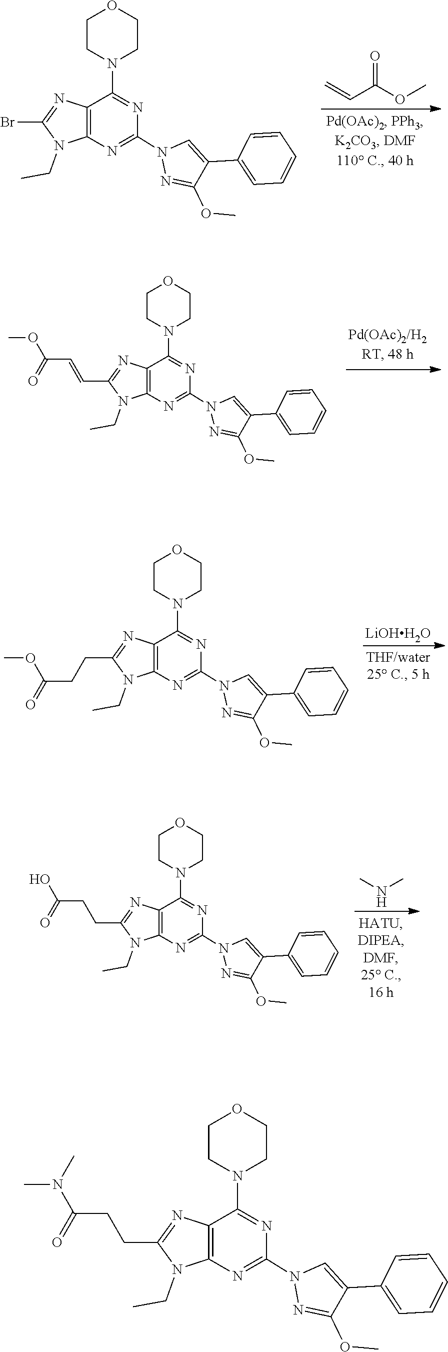

### Step 1: Synthesis of methyl (E)-3-(9-ethyl-2-(3-methoxy-4-phenyl-1H-pyrazol-1-yl)-6-morpholino-9H-purin-8-yl)acrylate

Palladium(II)acetate (27 mg, 0.12 mmol), triphenylphosphine (57 mg, 0.22 mmol) and potassium carbonate (260 mg, 1.86 mmol) were sequentially added to a degassed (with nitrogen for 30 min) solution of 4-(8-bromo-9-ethyl-2-(3-methoxy-4-phenyl-1H-pyrazol-1-yl)-9H-purin-6-yl)morpholine (0.3 g, 0.62 mmol) and methyl acrylate (0.27 g, 3.1 mmol) in DMF (10 mL). The resultant mixture was heated at 110° C. for 40 h. The reaction mixture was cooled down and used directly in next step without further purification. LCMS (ESI) m/z: 490.1 [M+H]⁺.

### Step 2: Synthesis of methyl 3-(9-ethyl-2-(3-methoxy-4-phenyl-1H-pyrazol-1-yl)-6-morpholino-9H-purin-8-yl)propanoate

The reaction mixture containing methyl (E)-3-(9-ethyl-2-(3-methoxy-4-phenyl-1H-pyrazol-1-yl)-6-morpholino-9H-purin-8-yl)acrylate from the above step (0.3 g, 0.62 mmol) in DMF (10 mL) was stirred under hydrogen atmosphere for 48 h at room temperature. Then water (15 mL) and ethyl acetate (25 mL) were added and stirred for 2 min. The organic layer was separated and aqueous layer was further extracted with ethyl acetate (3×25 mL). The combined organic extracts were dried over sodium sulfate, filtered and concentrated. The resultant residue was subjected to flash column chromatography (dichloromethane:methanol=15:1) to obtain methyl 3-(9-ethyl-2-(3-methoxy-4-phenyl-1H-pyrazol-1-yl)-6-morpholino-9H-purin-8-yl)propanoate (0.15 g, 50%) as yellow solid. LCMS (ESI) m/z: 492.1 [M+H]⁺.

### Step 3: Synthesis of 3-(9-ethyl-2-(3-methoxy-4-phenyl-1H-pyrazol-1-yl)-6-morpholino-9H-purin-8-yl)propanoic acid

A mixture of methyl 3-(9-ethyl-2-(3-methoxy-4-phenyl-1H-pyrazol-1-yl)-6-morpholino-9H-purin-8-yl)propanoate (0.12 g, 0.24 mmol) and lithium hydroxide hydrate (0.021 g, 0.49 mmol) in water/THF (1 mL/4 mL) was stirred at 25° C. for 5 h. It was then concentrated to obtain 3-(9-ethyl-2-(3-methoxy-4-phenyl-1H-pyrazol-1-yl)-6-morpholino-9H-purin-8-yl)propanoic acid (0.116 g, 100%) as yellow solid. LCMS (ESI) m/z: 478.1 [M+H]⁺.

### Step 4: Synthesis of 3-(9-ethyl-2-(3-methoxy-4-phenyl-1H-pyrazol-1-yl)-6-morpholino-9H-purin-8-yl)-N,N-dimethylpropanamide

A solution of 3-(9-ethyl-2-(3-methoxy-4-phenyl-1H-pyrazol-1-yl)-6-morpholino-9H-purin-8-yl)propanoic acid (100 mg, 0.21 mmol), dimethylamine (86 mg, 1.05 mmol), DIPEA (170 mg, 1.68 mmol) and 1-[Bis(dimethylamino)methylene]-1H-1,2,3-triazolo[4,5-b]pyridinium 3-oxid hexafluorophosphate (118 mg, 0.31 mmol) in DMF (5 mL) was stirred at 25° C. for 16 h. The resultant reaction mixture was filtered and the filtrate was subjected to prep-HPLC (BOSTON pHlex ODS 10 μm 21.2×250 mm 120 A, with mobile phase acetonitrile/0.1% ammonium bicarbonate) to obtain 3-(9-ethyl-2-(3-methoxy-4-phenyl-1H-pyrazol-1-yl)-6-morpholino-9H-purin-8-yl)-N,N-dimethylpropanamide as white solid (28.0 mg, 27%). ¹H NMR (400 MHz, DMSO-d₆) δ 8.91 (s, 1H), 7.80 (d, J=7.3 Hz, 2H), 7.39 (t, J=7.7 Hz, 2H), 7.24 (t, J=7.4 Hz, 1H), 4.25 (bs, 4H), 4.23 (q, J=7.2 Hz, 2H), 4.07 (s, 3H), 3.81-3.70 (m, 4H), 3.07 (t, J=6.8 Hz, 2H), 3.03 (s, 3H), 2.86 (d, J=6.7 Hz, 2H), 2.83 (s, 3H), 1.36 (t, J=7.2 Hz, 3H). LCMS (ESI) m/z: 505.0 [M+H]⁺.

### Synthesis of 2-(3-(4-bromo-1-methyl-1H-pyrazol-3-yl)phenyl)-9-ethyl-6-(pyridin-4-yl)-9H-purine (Compound 380)

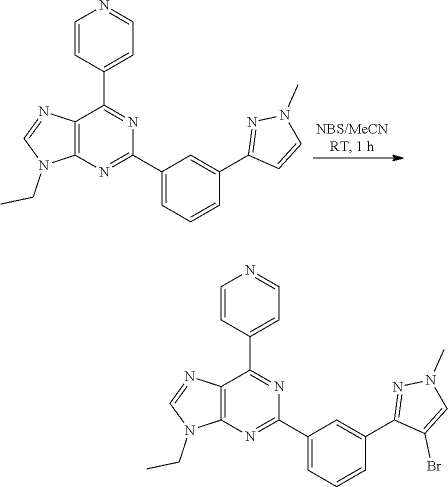 [SMILES: CCn1cnc2c(-c3ccncc3)nc(-c3cccc(-c4ccn(C)n4)c3)nc21 ; CCn1cnc2c(-c3ccncc3)nc(-c3cccc(-c4nn(C)cc4Br)c3)nc21]

A mixture of 9-ethyl-2-(3-(1-methyl-1H-pyrazol-3-yl)phenyl)-6-(pyridin-4-yl)-9H-purine (50 mg, 0.13 mmol) and N-bromosuccinimide (23 mg, 0.13 mmol) in acetonitrile (5 mL) was stirred at 20° C. for 1 h. The resultant mixture was poured into water and extracted with dichloromethane (100 mL*2). The combined organic phase was concentrated and the residue obtained was subjected to silica gel column chromatography (10% methanol in dichloromethane) and then to prep-HPLC (Column Xbridge 21.2*250 mm C18, 10 um, Mobile Phase A: water (10 mmol/L ammonium bicarbonate) to obtain 2-(3-(4-bromo-1-methyl-1H-pyrazol-3-yl)phenyl)-9-ethyl-6-(pyridin-4-yl)-9H-purine (45.4 mg, 76.1%) as off-white solid. ¹H NMR (500 MHz, CDCl₃) δ 9.23 (t, J=1.6 Hz, 1H), 8.86 (dd, J=4.5, 1.5 Hz, 2H), 8.82 (dd, J=4.5, 1.6 Hz, 2H), 8.69-8.62 (m, 1H), 8.17 (s, 1H), 8.02-7.97 (m, 1H), 7.60 (t, J=7.7 Hz, 1H), 7.53 (s, 1H), 4.46 (q, J=7.4 Hz, 2H), 3.99 (s, 3H), 1.66 (t, J=7.4 Hz, 3H); LCMS (ESI) m/z: 461.5, 462.6 [M+H]⁺. The regioselectivity of the bromination was confirmed by 2D-NMR.

### Synthesis of 9-ethyl-2-(3-methoxy-4-phenyl-1H-pyrazol-1-yl)-6-(piperidin-1-yl)-9H-purine (Compound 381)

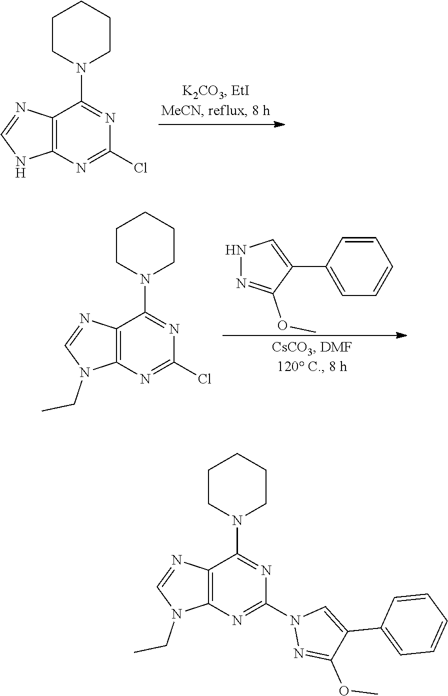

### Step 1: Synthesis of 2-chloro-9-ethyl-6-(piperidin-1-yl)-9H-purine

To a solution of 2-chloro-6-(piperidin-1-yl)-9H-purine (7.3 g, 0.0309 mol) in acetonitrile (100 mL) were added iodoethane (7.1 g, 45.8 mmol) and potassium carbonate (8.6 g, 61.8 mmol). The mixture was stirred at 90° C. for 8 h, then quenched with water (15 mL) and extracted with ethyl acetate (20 mL*3). The organic layers were combined, washed with brine (30 mL), dried over sodium sulfate, filtered and concentrated. The residue was then subjected to flash chromatography on silica gel (petroleum ether:ethyl acetate=75:25) to obtain 2-chloro-9-ethyl-6-(piperidin-1-yl)-9H-purine as yellow solid. (5.4 g, 65.9%). LCMS (ESI) m/z: 265.9[M]⁺.

### Step 2: Synthesis of 9-ethyl-2-(3-methoxy-4-phenyl-1H-pyrazol-1-yl)-6-(piperidin-1-yl)-9H-purine

To a solution of 2-chloro-9-ethyl-6-(piperidin-1-yl)-9H-purine (133 mg, 0.50 mmol) in DMF (5 mL) were added 3-methoxy-4-phenyl-1H-pyrazole (170 mg, 0.50 mmol) and cesium carbonate (32 mg, 1.0 mmol). The resultant mixture was stirred at 120° C. for 8 h, then quenched with water (15 mL) and extracted with ethyl acetate (20 mL*3). The organic layers were combined, washed with brine (30 mL), dried over sodium sulfate, filtered and concentrated. The residue was subjected to prep-HPLC (0.05% ammonium bicarbonate:acetonitrile=5%-95%) to obtain 9-ethyl-2-(3-methoxy-4-phenyl-1H-pyrazol-1-yl)-6-(piperidin-1-yl)-9H-purine (43.6 mg, 21.6%) as white solid. ¹H NMR (400 MHz, DMSO-d₆) δ 8.90 (s, 1H), 8.17 (s, 1H), 7.82 (d, J=7.3 Hz, 2H), 7.40 (t, J=7.7 Hz, 2H), 7.24 (t, J=7.4 Hz, 1H), 4.35 (bs, 4H), 4.21 (q, J=7.3 Hz, 2H), 4.07 (s, 3H), 1.79-1.57 (m, 6H), 1.45 (t, J=7.3 Hz, 3H). LCMS (ESI) m/z: 403.8[M]⁺.

### Synthesis of 4-(9-ethyl-2-(3-(pyridazin-3-yl)phenyl)-9H-purin-6-yl)morpholine (Compound 382)

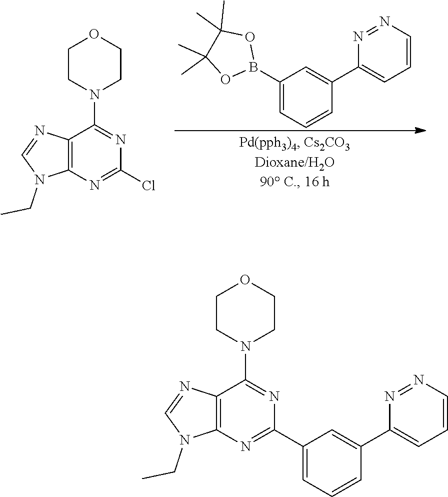 [SMILES: CCn1cnc2c(N3CCOCC3)nc(Cl)nc21 ; CC1(C)OB(c2cccc(-c3cccnn3)c2)OC1(C)C ; CCn1cnc2c(N3CCOCC3)nc(-c3cccc(-c4cccnn4)c3)nc21]

A solution of 4-(2-chloro-9-ethyl-9H-purin-6-yl)morpholine (54 mg, 0.2 mmol), 3-(3-(4,4,5,5-tetramethyl-1,3,2-dioxaborolan-2-yl)phenyl)pyridazine (84 mg, 0.3 mmol), cesium carbonate (163 mg, 0.5 mmol) and tetrakis(triphenylphosphine)palladium (23 mg, 0.02 mmol) in 1,4-dioxane/water (1 0 mL/1 mL) was stirred at 90° C. for 16 h. The resultant reaction mixture was filtered and the filtrate was concentrated. The residue was then subjected to prep-HPLC (BOSTON pHlex ODS 10 um 21.2×250 mm 120 A). The mobile phase was acetonitrile/0.1% ammonium bicarbonate) to obtain 4-(9-ethyl-2-(3-(pyridazin-3-yl)phenyl)-9H-purin-6-yl)morpholine (21.9 mg, 20.50%) as white solid. ¹H NMR (400 MHz, DMSO-d₆) δ 9.26 (dd, J=4.9, 1.5 Hz, 1H), 9.15 (d, J=1.6 Hz, 1H), 8.57 (d, J=7.8 Hz, 1H), 8.30 (dd, J=9.0, 1.8 Hz, 2H), 8.19 (d, J=8.2 Hz, 1H), 7.84 (dd, J=8.6, 4.9 Hz, 1H), 7.68 (t, J=7.8 Hz, 1H), 4.34 (bs, 4H), 4.31 (q, J=7.3 Hz, 2H), 3.79 (t, J=4.0 Hz, 4H), 1.49 (t, J=7.3 Hz, 3H). LCMS (ESI) m/z: 388.2 [M+H]⁺.

### Synthesis of 4-(2-(3-(1-methyl-1H-pyrazol-3-yl)phenyl)-9-(pyridin-3-ylmethyl)-9H-purin-6-yl)morpholine (Compound 383)

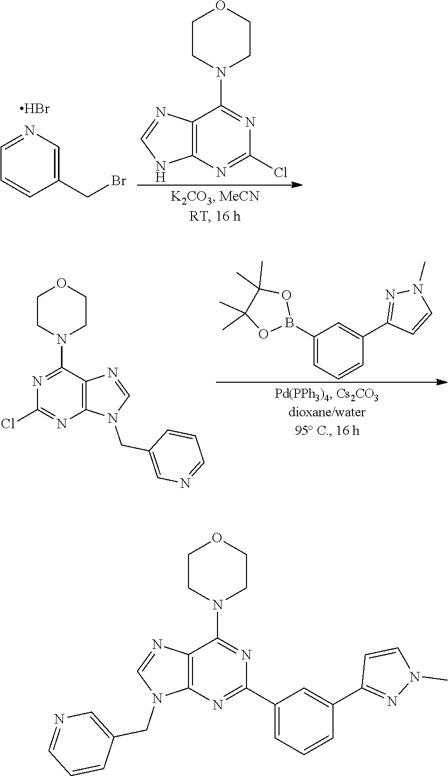

### Step 1: Synthesis of 4-(2-chloro-9-(pyridin-3-ylmethyl)-9H-purin-6-yl)morpholine

A mixture of 3-(bromomethyl)pyridine (253 mg, 1 mmol), 4-(2-chloro-9H-purin-6-yl)morpholine (432 mg, 1.5 mmol) and potassium carbonate (414 mg, 3 mmol) in acetonitrile (10 mL) was stirred at 25° C. for 16 h. The mixture was filtered to remove the solids and the filtrate was concentrated to obtain 4-(2-chloro-9-(pyridin-3-ylmethyl)-9H-purin-6-yl)morpholine (554 mg), which was used directly in next step without further purification. LCMS (ESI) m/z: 331.2 [M+H]⁺.

### Step 2: Synthesis of 4-(2-(3-(1-methyl-1H-pyrazol-3-yl)phenyl)-9-(pyridin-3-ylmethyl)-9H-purin-6-yl)morpholine

A mixture of 4-(2-chloro-9-(pyridin-3-ylmethyl)-9H-purin-6-yl)morpholine (554 mg, 1.38 mmol), 1-methyl-3-(3-(4,4,5,5-tetramethyl-1,3,2-dioxaborolan-2-yl)phenyl)-1H-pyrazole (392 mg, 1.38 mmol), tetrakis(triphenylphosphine)palladium(0) (239 mg, 0.207 mmol) and cesium carbonate (1.35 g, 4.14 mmol) in 1,4-dioxane/water (10 mL/2 mL) was stirred at 95° C. under argon atmosphere for 16 h. The reaction mixture was concentrated and the residue was subjected to silica gel column chromatography (petroleum ether:ethyl acetate=85:15) to obtain 4-(2-(3-(1-methyl-1H-pyrazol-3-yl)phenyl)-9-(pyridin-3-ylmethyl)-9H-purin-6-yl)morpholine as white solid. (180.8 mg, 29%). ¹H NMR (400 MHz, DMSO-d₆) δ 8.82-8.73 (m, 2H), 8.51 (dd, J=4.8, 1.5 Hz, 1H), 8.40 (s, 1H), 8.31 (d, J=7.8 Hz, 1H), 7.86 (ddd, J=7.8, 4.8, 1.7 Hz, 2H), 7.79 (d, J=2.2 Hz, 1H), 7.50 (t, J=7.7 Hz, 1H), 7.39 (dd, J=7.8, 4.8 Hz, 1H), 6.77 (d, J=2.2 Hz, 1H), 5.55 (s, 2H), 4.31 (bs, 4H), 3.92 (s, 3H), 3.82-3.75 (m, 4H). LCMS (ESI) m/z: 453.3 [M+H]⁺.

### Synthesis of (4-(9-ethyl-2-(3-(1-methyl-1H-pyrazol-3-yl)phenyl)-8-(pyridin-4-yl)-9H-purin-6-yl)morpholin-3-yl)methanol (Compound 384)

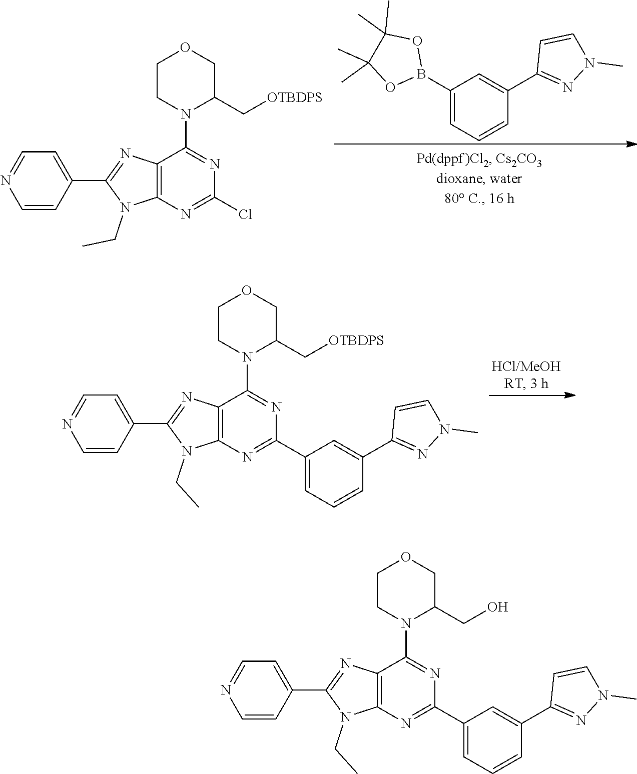

### Step 1: Synthesis of 3-(((tert-butyldiphenylsilyl)oxy)methyl)-4-(9-ethyl-2-(3-(1-methyl-1H-pyrazol-3-yl)phenyl)-8-(pyridin-4-yl)-9H-purin-6-yl)morpholine

A mixture of 3-(((tert-butyldiphenylsilyl)oxy)methyl)-4-(2-chloro-9-ethyl-8-(pyridin-4-yl)-9H-purin-6-yl)morpholine (240 mg, 0.4 mmol), 1-methyl-3-(3-(4,4,5,5-tetramethyl-1,3,2-dioxaboro lan-2-yl)phenyl)-1H-pyrazole (166 mg, 0.59 mmol), [1,1′-bis(diphenylphosphino)ferrocene]dichloropalladium(II) (30 mg, 0.04 mmol) and cesium carbonate (260 mg, 0.78 mmol) in 1,4-dioxane (5 mL) and water (0.5 mL) was stirred at 85° C. under argon for 16 h. It was filtered and the filtrate was concentrated. The residue was subjected to column chromatography on silica gel (dichloromethane/methanol=10/1) to obtain 3-(((tert-butyldiphenylsilyl)oxy)methyl)-4-(9-ethyl-2-(3-(1-methyl-1H-pyrazol-3-yl)phenyl)-8-(pyridin-4-yl)-9H-purin-6-yl)morpholine (200 mg, 68%) as white solid. LCMS (ESI) m/z: 735.4 [M+H]⁺

### Step 2: Synthesis of (4-(9-ethyl-2-(3-(1-methyl-1H-pyrazol-3-yl)phenyl)-8-(pyridin-4-yl)-9H-purin-6-yl)morpholin-3-yl)methanol

A solution of 3-(((tert-butyldiphenylsilyl)oxy)methyl)-4-(9-ethyl-2-(3-(1-methyl-1H-pyrazol-3-yl)phenyl)-8-(pyridin-4-yl)-9H-purin-6-yl)morpholine (200 mg, 0.273 mmol) in methanolic hydrochloric acid solution (5 mL) was stirred at room temperature for 2 h. Then water was added and the mixture was extracted with ethyl acetate (50 mL×3). The organic layer was dried over sodium sulfate and concentrated. The residue obtained was subjected to prep-HPLC to give (4-(9-ethyl-2-(3-(1-methyl-1H-pyrazol-3-yl)phenyl)-8-(pyridin-4-yl)-9H-purin-6-yl)morpholin-3-yl)methanol (127.6 mg, 95%) as white solid. ¹H NMR (400 MHz, DMSO-d₆) δ 8.83-8.77 (m, 3H), 8.35 (d, J=7.9 Hz, 1H), 7.87 (dd, J=7.1, 4.0 Hz, 3H), 7.79 (d, J=2.2 Hz, 1H), 7.52 (t, J=7.7 Hz, 1H), 6.76 (d, J=2.2 Hz, 1H), 5.76 (bs, 1H), 4.97 (s, 2H), 4.51 (q, J=7.3 Hz, 2H), 4.14 (d, J=11.3 Hz, 1H), 4.02 (d, J=8.9 Hz, 1H), 3.94-3.86 (m, 4H), 3.74-3.37 (m, 4H), 1.40 (t, J=7.2 Hz, 3H). LCMS (ESI) m/z: 496.8 [M]⁺.

### Synthesis of 1-(9-ethyl-2-(3-methoxy-4-phenyl-1H-pyrazol-1-yl)-9H-purin-6-yl)piperidin-4-ol (Compound 385)

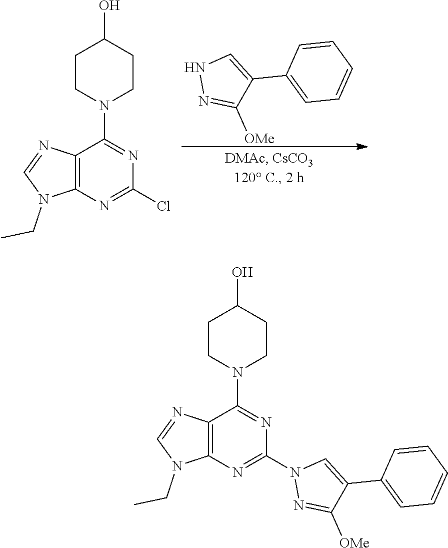 [SMILES: CCn1cnc2c(N3CCC(O)CC3)nc(Cl)nc21 ; COc1n[nH]cc1-c1ccccc1 ; CCn1cnc2c(N3CCC(O)CC3)nc(-n3cc(-c4ccccc4)c(OC)n3)nc21]

To a solution of 1-(2-chloro-9-ethyl-9H-purin-6-yl)piperidin-4-ol (200 mg, 0.71 mmol) in N,N-dimethylformamide (5 mL) was added 3-methoxy-4-phenyl-1H-pyrazole (148.4 mg, 0.85 mmol) and cesium carbonate (693.83 mg, 2.13 mmol) and the reaction mixture was stirred at 120° C. for 16 h under nitrogen atmosphere. It was then extracted with dichloromethane (20 mL*2) and the combined extracts were washed with water (10 mL*2). The organic layer was dried over sodium sulfate, filtered and concentrated. The residue was subjected to silica gel column chromatography (2% methanol in dichloromethane) to obtain 1-(9-ethyl-2-(3-methoxy-4-phenyl-1H-pyrazol-1-yl)-9H-purin-6-yl)piperidin-4-ol as white solid (25.6 mg, 8.6%). ¹H NMR (400 MHz, DMSO-d₆) δ 8.91 (s, 1H), 8.20 (s, 1H), 7.82 (d, J=7.6 Hz, 2H), 7.40 (t, J=7.6 Hz, 2H), 7.25 (t, J=7.3 Hz, 1H), 4.94 (bs, 3H), 4.25 (bs, 2H), 4.09 (bs, 3H), 3.82 (bs, 3H), 1.90 (d, J=11.1 Hz, 2H), 1.47 (s, 5H). LCMS (ESI) m/z: 420.3 [M+H]⁺.

### Synthesis of 4-(9-ethyl-2-(3-methoxy-4-phenyl-1H-pyrazol-1-yl)-8-methyl-9H-purin-6-yl)morpholine (Compound 386)

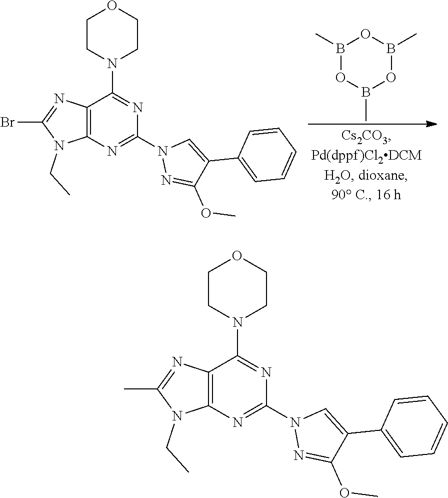

A mixture of 4-(8-bromo-9-ethyl-2-(3-methoxy-4-phenyl-1H-pyrazol-1-yl)-9H-purin-6-yl)morpholine (148 mg, 0.3 mmol), 2,4,6-trimethyl-1,3,5,2,4,6-trioxatriborinane (38.5 mg, 0.3 mmol), 1,1′-bis(diphenylphosphino)ferrocene-palladium(II)dichloride dichloromethane complex (25 mg, 0.03 mmol) and cesium carbonate (300 mg, 0.9 mmol) in 1,4-dioxane (10 mL) with water (2 mL) was stirred at 90° C. under argon atmosphere for 16 h. The resultant reaction mixture was filtered and the filtrate was subjected to prep-HPLC (Column Xbridge 21.2*250 mm C18, 10 um, Mobile Phase A: water (10 mmol/L ammonium bicarbonate) B: acetonitrile) to obtain 4-(9-ethyl-2-(3-methoxy-4-phenyl-1H-pyrazol-1-yl)-8-methyl-9H-purin-6-yl)morpholine (69 mg, 55%) as white solid. ¹H NMR (400 MHz, DMSO-d₆) δ 8.91 (s, 1H), 7.81 (d, J=7.2 Hz, 2H), 7.39 (t, J=7.7 Hz, 2H), 7.24 (t, J=7.4 Hz, 1H), 4.28 (bs, 4H), 4.21 (q, J=7.2 Hz, 2H), 4.07 (s, 3H), 3.80-3.70 (m, 4H), 2.53 (s, 3H), 1.34 (t, J=7.2 Hz, 3H). LCMS (ESI) m/z: 420.3 [M+H]⁺.

### Synthesis of 8-butyl-9-ethyl-2-(3-(1-methyl-1H-pyrazol-3-yl)phenyl)-6-(pyridin-4-yl)-9H-purine (Compound 387)

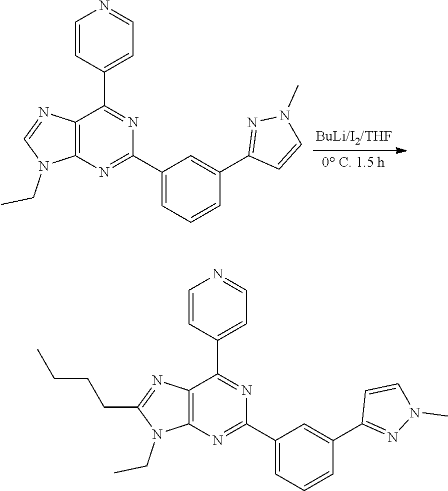 [SMILES: CCn1cnc2c(-c3ccncc3)nc(-c3cccc(-c4ccn(C)n4)c3)nc21 ; CCCCc1nc2c(-c3ccncc3)nc(-c3cccc(-c4ccn(C)n4)c3)nc2n1CC]

To a solution of 9-ethyl-2-(3-(1-methyl-1H-pyrazol-3-yl)phenyl)-6-(pyridin-4-yl)-9H-purine (76 mg, 0.2 mmol) in tetrahydrofuran (5.0 mL) was added dropwise a solution of n-butyllithium (2.5M in tetrahydrofuran, 0.1 mL) at 0° C. After the addition, the reaction mixture was stirred at 0° C. for another 30 min. Then iodine (50 mg, 0.2 mmol) was added and the mixture was stirred at 0° C. for another 1 h. The reaction was quenched with crushed ice and extracted with ethyl acetate (100 mL*2). The combined organic phase was concentrated and the residue was subjected to silica gel column chromatography (5% methanol in dichloromethane) and then to prep-HPLC (Column Xbridge 21.2*250 mm C18, 10 um, Mobile Phase A: water (10 mmol/L ammonium bicarbonate) to obtain 8-butyl-9-ethyl-2-(3-(1-methyl-1H-pyrazol-3-yl)phenyl)-6-(pyridin-4-yl)-9H-purine (24.5 mg, 28%) as grey solid. ¹H NMR (400 MHz, CDCl₃) δ 9.02 (t, J=1.6 Hz, 1H), 8.85 (s, 4H), 8.62-8.56 (m, 1H), 7.97-7.91 (m, 1H), 7.56 (t, J=7.7 Hz, 1H), 7.45 (d, J=2.2 Hz, 1H), 6.71 (d, J=2.2 Hz, 1H), 4.42 (q, J=7.2 Hz, 2H), 4.02 (s, 3H), 2.99 (t, J=8.0 Hz, 2H), 1.98 (dt, J=15.5, 7.6 Hz, 2H), 1.60-1.51 (m, 5H), 1.05 (t, J=7.4 Hz, 3H); LCMS (ESI) m/z: 437.9 [M]⁺.

### Synthesis of 1-[3-[9-ethyl-6-morpholino-8-(4-pyridyl)purin-2-yl]phenyl]pyrrolidin-2-one (Compound 388)

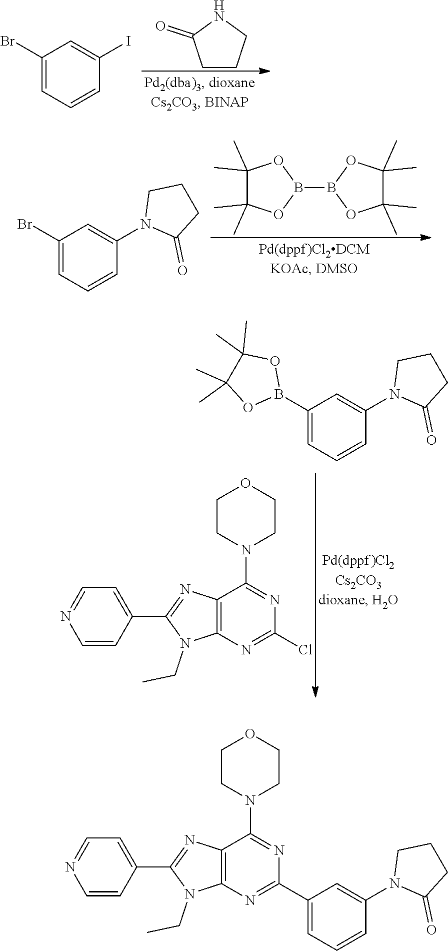

### Step 1: Synthesis of 1-(3-bromophenyl)pyrrolidin-2-one

To a solution of 1-bromo-3-iodo-benzene (2 g, 7.07 mmol) and pyrrolidin-2-one (602 mg, 7.07 mmol) in dioxane (15 mL) were added Cs₂CO₃ (6.91 g, 21.21 mmol), BINAP (440 mg, 707 umol) and Pd₂(dba)₃ (324 mg, 353 umol). The mixture was stirred at 100° C. for 3 h under nitrogen atmosphere and then quenched by 15 mL of water. It was extracted with ethyl acetate (10 mL*3), the combined organic layers were dried over Na₂SO₄ and concentrated. The residue was subjected to flash column chromatography (ISCO 2 g silica, 0-45% ethyl acetate in petroleum ether, gradient over 50 min) to obtain 1-(3-bromophenyl)pyrrolidin-2-one (400 mg, 23%) as brown oil. LCMS (ESI) m/z: 240.9 [M+H]⁺. ¹H NMR (400 MHz, CHLOROFORM-d) δ 7.81 (t, J=2.0 Hz, 1H), 7.65-7.59 (m, 1H), 7.30-7.27 (m, 1H), 7.26-7.19 (m, 1H), 3.85 (t, J=7.0 Hz, 2H), 2.63 (t, J=8.1 Hz, 2H), 2.24-2.12 (m, 2H).

### Step 2: Synthesis of 1-[3-(4,4,5,5-tetramethyl-1,3,2-dioxaborolan-2-yl)phenyl]pyrrolidin-2-one

To a solution of 1-(3-bromophenyl)pyrrolidin-2-one (370 mg, 1.54 mmol) and 4,4,5,5-tetramethyl-2-(4,4,5,5-tetramethyl-1,3,2-dioxaborolan-2-yl)-1,3,2-dioxaborolane (783 mg, 3.08 mmol) in DMSO (6 mL) were added KOAc (454 mg, 4.62 mmol) and [1,1′-bis(diphenylphosphino)ferrocene]dichloropalladium(II) (1:1)⁻ DCM (56 mg, 77 umol). The resultant mixture was stirred at 90° C. for 12 h followed by the addition of 7 mL of water. The mixture was then extracted with ethyl acetate (8 mL*3), the combined organic layers was dried over Na₂SO₄ and concentrated. The residue was subjected to flash column chromatography (ISCO 4 g silica, 0-32% ethyl acetate in petroleum ether, gradient over 25 min) to obtain 1-[3-(4,4,5,5-tetramethyl-1,3,2-dioxaborolan-2-yl)phenyl]pyrrolidin-2-one (387 mg, 87%) as brown oil. LCMS (ESI) m/z: 288.1 [M+H]⁺

### Step 3: Synthesis of 1-[3-[9-ethyl-6-morpholino-8-(4-pyridyl)purin-2-yl]phenyl]pyrrolidin-2-one

To a solution of 4-[2-chloro-9-ethyl-8-(4-pyridyl)purin-6-yl]morpholine (50 mg, 145 umol) and 1-[3-(4,4,5,5-tetramethyl-1,3,2-dioxaborolan-2-yl)phenyl]pyrrolidin-2-one (42 mg, 145 umol) in dioxane (1 mL) and H₂O (0.1 mL) were added Pd(dppf)Cl₂ (11 mg, 15 umol) and Cs₂CO₃ (142 mg, 435 umol). The mixture was stirred at 100° C. for 2 h under argon atmosphere. The reaction mixture was filtered and the filtrate was subjected to prep-HPLC (Phenomenex luna C18 80*40 mm*3 um; 22-48% acetonitrile in a hydrochloric acid solution in water, 7 min gradient) to obtain 1-[3-[9-ethyl-6-morpholino-8-(4-pyridyl)purin-2-yl]phenyl]pyrrolidin-2-one (10 mg, 15%) as yellow solid. ¹H NMR of YUMAX-07122-04-01 (400 MHz, CHLOROFORM-d) δ 8.87 (bs, 2H), 8.76 (s, 1H), 8.35 (bs, 2H), 8.26 (d, J=7.7 Hz, 1H), 7.69 (d, J=7.5 Hz, 1H), 7.48 (t, J=7.9 Hz, 1H), 4.66 (bs, 2H), 4.47 (bs, 4H), 4.00 (t, J=6.8 Hz, 2H), 3.92 (bs, 4H), 2.69 (t, J=8.1 Hz, 2H), 2.24 (quin, J=7.3 Hz, 2H), 1.62 (bs, 3H). (ESI for C₂₆H₂₇N₇O₂) m/z: 470.1 [M+H]⁺.

### Synthesis of 4-[3-[9-ethyl-6-morpholino-8-(4-pyridyl)purin-2-yl]phenyl]morpholin-3-one (Compound 389)

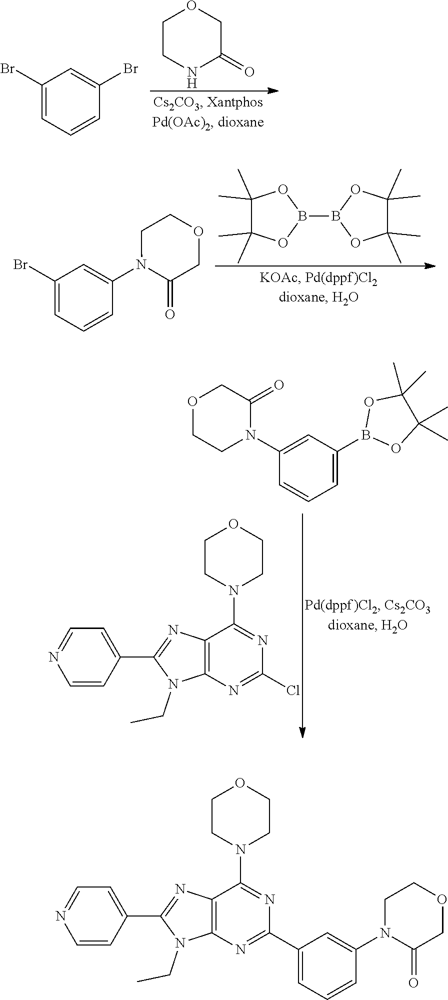

### Step 1: Synthesis of 4-(3-bromophenyl)morpholin-3-one

To a solution of 1,3-dibromobenzene (2 g, 8.48 mmol) and morpholin-3-one (686 mg, 6.78 mmol) in dioxane (25 mL) were added Cs₂CO₃ (5.52 g, 16.96 mmol), Xantphos (736 mg, 1.27 mmol), and Pd(OAc)₂ (190 mg, 848 umol). The resultant mixture was stirred at 100° C. for 4 h followed by the addition of 30 mL and the mixture was extracted with ethyl acetate (30 mL*3). The combined organic layers was dried over Na₂SO₄ and concentrated. The residue was subjected to flash column chromatography (ISCO 2 g silica, 0-50% ethyl acetate in petroleum ether, gradient over 50 min) to obtain 4-(3-bromophenyl)morpholin-3-one (912 mg, 42%) as brown oil. LCMS (ESI) m/z: 255.9 [M+H]⁺

### Step 2: Synthesis of 4-[3-(4,4,5,5-tetramethyl-1,3,2-dioxaborolan-2-yl)phenyl]morpholin-3-one

To a solution of 4,4,5,5-tetramethyl-2-(4,4,5,5-tetramethyl-1,3,2-dioxaborolan-2-yl)-1,3,2-dioxaborolane (1.69 g, 6.64 mmol) and 4-(3-bromophenyl)morpholin-3-one (850 mg, 3.32 mmol) in dioxane (10 mL) were added KOAc (977 mg, 9.96 mmol) and [1,1′-bis(diphenylphosphino)ferrocene]dichloropalladium(II) (1:1)⁻ DCM (121 mg, 166 umol). The mixture was then stirred at 100° C. for 12 h followed by the addition of 10 mL of water and the mixture was extracted with ethyl acetate (10 mL*3). The combined organic layers was dried over Na₂SO₄ and concentrated. The residue was subjected to flash column chromatography (ISCO 2 g silica, 0-53% ethyl acetate in petroleum ether, gradient over 20 min) to obtain 4-[3-(4,4,5,5-tetramethyl-1,3,2-dioxaborolan-2-yl)phenyl]morpholin-3-one (570 mg, 57%) as brown oil. LCMS (ESI) m/z: 304.1 [M+H]⁺

### Step 3: Synthesis of 4-[3-[9-ethyl-6-morpholino-8-(4-pyridyl)purin-2-yl]phenyl]morpholin-3-one

To a solution of 4-[2-chloro-9-ethyl-8-(4-pyridyl)purin-6-yl]morpholine (100 mg, 290 umol) and 4-[3-(4,4,5,5-tetramethyl-1,3,2-dioxaborolan-2-yl)phenyl]morpholin-3-one (88 mg, 290 umol) in dioxane (1.5 mL) and H₂O (0.2 mL) were added Cs₂CO₃ (283 mg, 870 umol) and Pd(dppf)Cl₂ (21 mg, 29 umol). The resultant mixture was stirred at 100° C. for 2 h and then filtered to remove the solids. The filtrate was then subjected to prep-HPLC (Phenomenex luna C18 80*40 mm*3 um; 20-50% acetonitrile in a hydrochloric acid solution in water, 8 min gradient) to obtain 4-[3-[9-ethyl-6-morpholino-8-(4-pyridyl)purin-2-yl]phenyl]morpholin-3-one (92 mg, 63%) as yellow solid. ¹H NMR (400 MHz, CHLOROFORM-d) δ 8.92 (bs, 2H), 8.48-8.27 (m, 4H), 7.53-7.46 (m, 1H), 7.46-7.39 (m, 1H), 4.76-4.27 (m, 8H), 4.11 (t, J=4.8 Hz, 2H), 3.98-3.81 (m, 6H), 1.59 (bs, 3H). (ESI for C26H27N7O3) m/z: 486.1 [M+H]⁺.

### Synthesis of 3-[9-ethyl-6,8-bis(4-pyridyl)purin-2-yl]-N,N-dimethyl-benzamide (Compound 390)

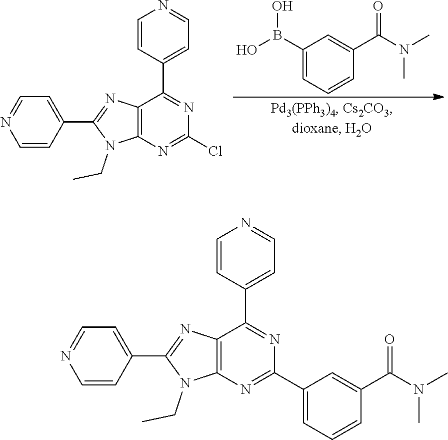 [SMILES: CCn1c(-c2ccncc2)nc2c(-c3ccncc3)nc(Cl)nc21 ; CN(C)C(=O)c1cccc(B(O)O)c1 ; CCn1c(-c2ccncc2)nc2c(-c3ccncc3)nc(-c3cccc(C(=O)N(C)C)c3)nc21]

To a solution of 2-chloro-9-ethyl-6,8-bis(4-pyridyl)purine (100 mg, 297 umol) and [3-(dimethylcarbamoyl)phenyl]boronic acid (57 mg, 297 umol) in dioxane (2 mL) and H₂O (0.2 mL) were added Cs₂CO₃ (193 mg, 594 umol) and Pd(PPh₃)₄ (17 mg, 15 umol). The resultant mixture was stirred at 100° C. for 2 h and then filtered to remove the solids. The filtrate was then subjected to prep-HPLC condition 1 (Phenomenex luna C18 80*40 mm*3 um; 26-40% acetonitrile in a 0.1% trifluoroacetic acid solution in water, 4 min gradient) and then to prep-HPLC condition 2 (Waters Xbridge Prep OBD C18 150*40 mm*10 um; 30-60% acetonitrile in a 10 mM sodium bicarbonate solution in water, 8 min gradient) to obtain 3-[9-ethyl-6,8-bis(4-pyridyl)purin-2-yl]-N,N-dimethyl-benzamide (18 mg, 13%) as white solid. ¹H NMR (400 MHz, CHLOROFORM-d) δ 9.00-8.85 (m, 6H), 8.80-8.72 (m, 2H), 7.88-7.80 (m, 2H), 7.65-7.52 (m, 2H), 4.61 (q, J=7.2 Hz, 2H), 3.21 (s, 3H), 3.09 (s, 3H), 1.61 (t, J=7.2 Hz, 3H). (ESI for C26H23N7O) m/z: 450.2 [M+H]⁺.

### Synthesis of (E)-1-(9-ethyl-2-(2-(3-methylbenzylidene)hydrazinyl)-8-(pyridin-4-yl)-9H-purin-6-yl)piperidin-4-ol (Compound 391)

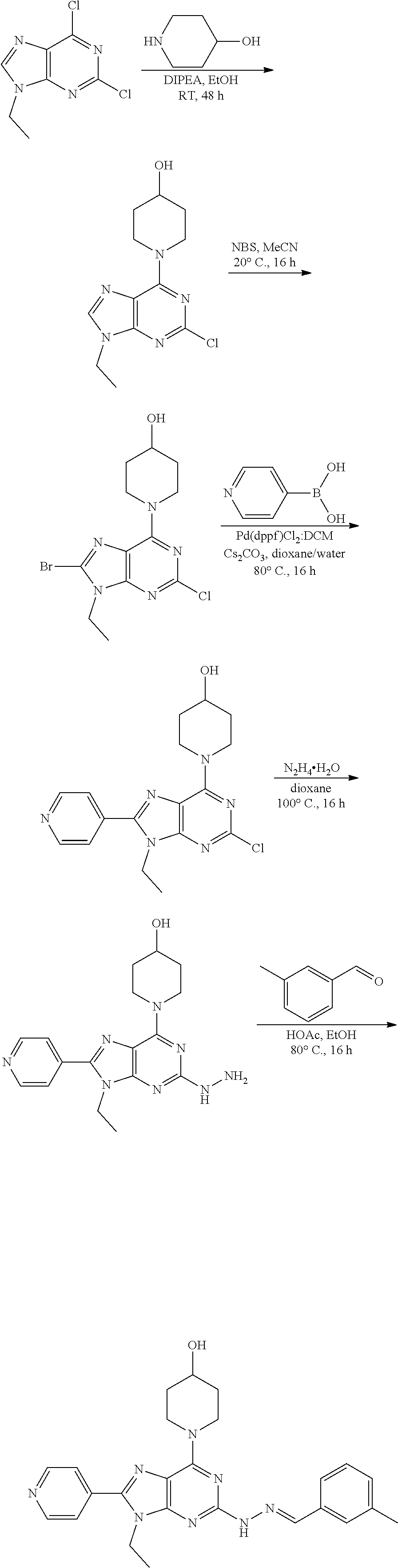

### Step 1: Synthesis of 1-(2-chloro-9-ethyl-9H-purin-6-yl)piperidin-4-ol

To a stirred solution of 2,6-dichloro-9-ethyl-9H-purine (260 mg, 1.2 mmol) and piperidin-4-ol (133 mg, 1.32 mmol) in ethanol (10 mL) was added N-ethyl-N-isopropylpropan-2-amine (155 mg, 1.2 mmol). The resultant reaction mixture was stirred at 20° C. for 48 h and concentrated. The residue was subjected to silica gel column chromatography (methanol/dichloromethane=0%-5%) to obtain 1-(2-chloro-9-ethyl-9H-purin-6-yl)piperidin-4-ol (300 mg, 87%) as white solid. LCMS (ESI) m/z: 282.1 [M+H]⁺.

### Step 2: Synthesis of 1-(8-bromo-2-chloro-9-ethyl-9H-purin-6-yl)piperidin-4-ol

A solution of 1-(2-chloro-9-ethyl-9H-purin-6-yl)piperidin-4-ol (246 mg, 0.87 mmol) and N-bromosuccinimide (233 mg, 1.31 mmol) in acetonitrile (10 mL) was stirred at 20° C. for 16 h. It was concentrated and the residue was subjected to silica gel column chromatography (methanol/dichloromethane=0%-5%) to obtain 1-(8-bromo-2-chloro-9-ethyl-9H-purin-6-yl)piperidin-4-ol (400 mg) as white solid. LCMS (ESI) m/z: 360.0 [M+H]⁺.

### Step 3: Synthesis of 1-(2-chloro-9-ethyl-8-(pyridin-4-yl)-9H-purin-6-yl)piperidin-4-ol

A solution of 1-(8-bromo-2-chloro-9-ethyl-9H-purin-6-yl)piperidin-4-ol (364 mg, 1 mmol), pyridin-4-ylboronic acid (123 mg, 1 mmol), 1,1′-bis(diphenylphosphino) ferrocene-palladium(II) dichloride dichloromethane complex (82 mg, 0.1 mmol) and cesium carbonate (978 mg, 3 mmol) in water (1 mL) and dioxane (10 mL) was stirred at 80° C. for 16 h under argon atmosphere. The reaction mixture was concentrated and the residue was subjected to silica gel column chromatography (methanol/dichloromethane=0%-7%) to obtain 1-(2-chloro-9-ethyl-8-(pyridin-4-yl)-9H-purin-6-yl)piperidin-4-ol (220 mg, 61%) as white solid. LCMS (ESI) m/z: 359.1 [M+H]⁺.

### Step 4: Synthesis of 1-(9-ethyl-2-hydrazinyl-8-(pyridin-4-yl)-9H-purin-6-yl)piperidin-4-ol

A mixture of 1-(2-chloro-9-ethyl-8-(pyridin-4-yl)-9H-purin-6-yl)piperidin-4-ol (36 mg, 0.1 mmol) and hydrazine hydrate (0.5 mL) in 1,4-dioxane (3 mL) was stirred at 100° C. for 16 h. The reaction mixture was concentrated to obtain 1-(9-ethyl-2-hydrazinyl-8-(pyridin-4-yl)-9H-purin-6-yl)piperidin-4-ol as yellow solid. (60 mg) which was used directly for the next step. LCMS (ESI) m/z: 355.1 [M+H]⁺.

### Step 5: Synthesis of (E)-1-(9-ethyl-2-(2-(3-methylbenzylidene)hydrazinyl)-8-(pyridin-4-yl)-9H-purin-6-yl)piperidin-4-ol

To a solution of 1-(9-ethyl-2-hydrazinyl-8-(pyridin-4-yl)-9H-purin-6-yl)piperidin-4-ol (40 mg, 0.1 mmol) and 3-methylbenzaldehyde (24 mg, 0.2 mmol) in ethanol (5 mL) was added acetic acid (one drop) and the resultant mixture was stirred at 80° C. for 16 h under argon atmosphere. It was concentrated and the residue was subjected to prep-HPLC (BOSTON pHlex ODS 10 μm 21.2×250 mm 120 A. The mobile phase was acetonitrile/0.1% ammonium bicarbonate) to obtain (E)-1-(9-ethyl-2-(2-(3-methylbenzylidene)hydrazinyl)-8-(pyridin-4-yl)-9H-purin-6-yl)piperidin-4-ol (16.2 mg, 24%) as yellow solid.

¹H NMR (400 MHz, DMSO-d₆) δ 10.79 (s, 1H), 8.76 (dd, J=4.6, 1.5 Hz, 2H), 8.08 (s, 1H), 7.82 (dd, J=4.6, 1.6 Hz, 2H), 7.51-7.39 (m, 2H), 7.30 (t, J=7.6 Hz, 1H), 7.15 (d, J=7.4 Hz, 1H), 4.86 (bs, 3H), 4.34 (q, J=7.2 Hz, 2H), 3.80 (s, 1H), 3.67 (bs, 2H), 2.35 (s, 3H), 1.93-1.83 (m, 2H), 1.49-1.38 (m, 2H), 1.32 (t, J=7.2 Hz, 3H). LCMS (ESI) m/z: 457.0 [M+H]⁺.

### Synthesis of (E)-9-ethyl-2-(2-(3-methylbenzylidene)hydrazinyl)-6-(piperidin-1-yl)-8-(pyridin-4-yl)-9H-purine (Compound 392)

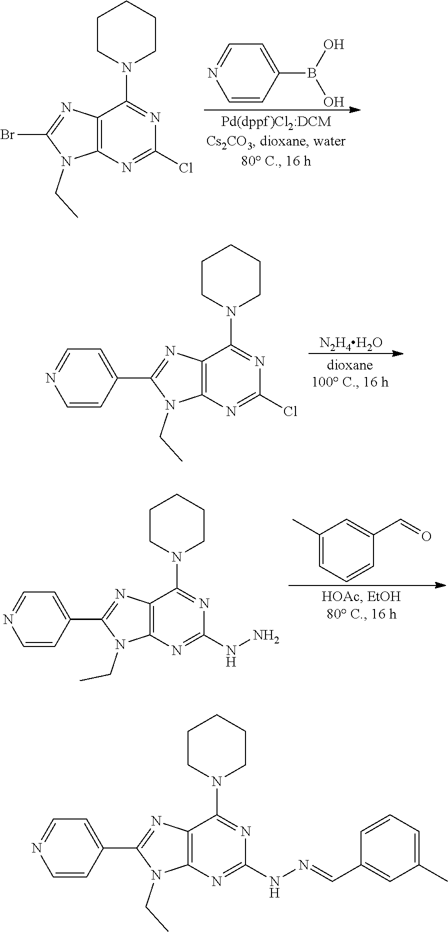

### Step 1: Synthesis of 2-chloro-9-ethyl-6-(piperidin-1-yl)-8-(pyridin-4-yl)-9H-purine

A solution of 8-bromo-2-chloro-9-ethyl-6-(piperidin-1-yl)-9H-purine (130 mg, 0.38 mmol), pyridin-4-ylboronic acid (46 mg, 0.38 mmol), 1,1′-bis(diphenylphosphino) ferrocene-palladium(II) dichloride dichloromethane complex (31 mg, 0.038 mmol) and cesium carbonate (372 mg, 1.14 mmol) in water (0.5 mL) and dioxane (5 mL) was stirred at 80° C. for 16 h under argon. The reaction mixture was cooled and concentrated. The resultant residue was subjected to flash chromatography (Biotage, 40 g silica gel, methanol in dichloromethane from 0%-5%) to obtain 2-chloro-9-ethyl-6-(piperidin-1-yl)-8-(pyridin-4-yl)-9H-purine (80 mg, 61%) as white solid. LCMS: (ESI) m/z: 343.1 [M+H]⁺.

### Step 2: Synthesis of 9-ethyl-2-hydrazinyl-6-(piperidin-1-yl)-8-(pyridin-4-yl)-9H-purine

A mixture of 2-chloro-9-ethyl-6-(piperidin-1-yl)-8-(pyridin-4-yl)-9H-purine (80 mg, 0.23 mmol) and hydrazine hydrate (0.5 mL) in dioxane (3 mL) was stirred at 100° C. for 16 h. It was concentrated to obtain 9-ethyl-2-hydrazinyl-6-(piperidin-1-yl)-8-(pyridin-4-yl)-9H-purine (90 mg) as yellow solid which was used directly in next step without further purification. LCMS (ESI) m/z: 338.9 [M+H]⁺.

### Step 3: Synthesis of (E)-9-ethyl-2-(2-(3-methylbenzylidene)hydrazinyl)-6-(piperidin-1-yl)-8-(pyridin-4-yl)-9H-purine

To a solution of 9-ethyl-2-hydrazinyl-6-(piperidin-1-yl)-8-(pyridin-4-yl)-9H-purine (90 mg, 0.23 mmol) and 3-methylbenzaldehyde (55 mg, 0.46 mmol) in ethanol (5 mL) was added acetic acid (one drop) and the mixture was stirred at 80° C. for 16 h under argon atmosphere. It was concentrated and the residue was subjected to prep-HPLC (BOSTON pHlex ODS 10 μm 21.2×250 mm 120 A. The mobile phase was acetonitrile/0.1% ammonium bicarbonate) to obtain (E)-9-ethyl-2-(2-(3-methylbenzylidene)hydrazinyl)-6-(piperidin-1-yl)-8-(pyridin-4-yl)-9H-purine (57.4 mg, 56%) as yellow solid.

¹H NMR (500 MHz, DMSO-d₆) δ 10.76 (s, 1H), 8.74 (d, J=5.9 Hz, 2H), 8.08 (s, 1H), 7.79 (d, J=5.9 Hz, 2H), 7.49-7.42 (m, 2H), 7.30 (t, J=7.6 Hz, 1H), 7.15 (d, J=7.5 Hz, 1H), 4.33 (q, J=7.2 Hz, 2H), 4.25 (bs, 4H), 2.34 (s, 3H), 1.69 (d, J=4.6 Hz, 2H), 1.62 (s, 4H), 1.31 (t, J=7.2 Hz, 3H); LCMS (ESI) m/z: 441.3 [M+H]⁺.

### Synthesis of 2-(9-ethyl-2-(3-methoxy-4-phenyl-1H-pyrazol-1-yl)-6-morpholino-9H-purin-8-yl)-2-methoxyethan-1-ol (Compound 55) and 1-(9-ethyl-2-(3-methoxy-4-phenyl-1H-pyrazol-1-yl)-6-morpholino-9H-purin-8-yl)-2-methoxyethan-1-ol (Compound 56)

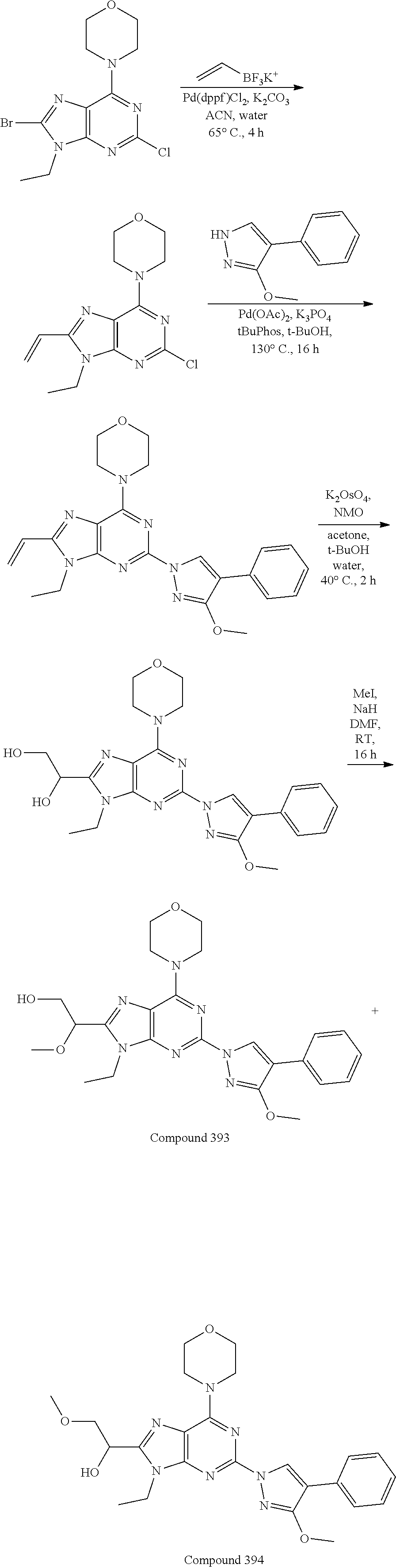

### Step 1: Preparation of 4-(2-chloro-9-ethyl-8-vinyl-9H-purin-6-yl)morpholine

To a solution of 4-(8-bromo-2-chloro-9-ethyl-9H-purin-6-yl)morpholine (3.5 g, 10.1 mmol) and trifluoro(vinyl)-l4-borane, potassium salt (1.35 g, 10.1 mmol) in acetonitrile/water (50 mL/10 mL) were added potassium carbonate (2.8 g, 20.2 mmol) and [1,1′-bis(diphenylphosphino)ferrocene]dichloropalladium(II) (0.73 g, 1.0 mmol) and the mixture was stirred at 90° C. for 2 h under argon. The reaction mixture was then concentrated and purified by silica gel column chromatography (ethyl acetate/petroleum ether=1:2) to give 4-(2-chloro-9-ethyl-8-vinyl-9H-purin-6-yl)morpholine as an yellow solid. (1.8 g, 61%). LCMS (ESI) m/z: 294.0 [M+H]⁺.

### Step 2: Preparation of 4-(9-ethyl-2-(3-methoxy-4-phenyl-1H-pyrazol-1-yl)-8-vinyl-9H-purin-6-yl)morpholine

To a mixture of 3-methoxy-4-phenyl-1H-pyrazole (1.1 g, 6.1 mmol) in tert-butyl alcohol (20 mL) were added 4-(2-chloro-9-ethyl-8-vinyl-9H-purin-6-yl)morpholine (1.2 g, 4.1 mmol), potassium phosphate tribasic (1.9 g, 9.0 mmol), tris(dibenzylideneacetone) dipalladium (0) (0.38 g, 0.41 mmol) and 2-di-t-butylphosphino-2′,4′,6′-tri-i-propyl-1,1′-biphenyl (0.35 g, 0.82 mmol) and the reaction mixture was stirred at 110° C. for 4 h under argon atmosphere. The reaction mixture was poured into water (30 mL), extracted with ethyl acetate (40 mL*3), concentrated and purified by silica gel column chromatography (ethyl acetate/petroleum ether=1:1) to afford 4-(9-ethyl-2-(3-methoxy-4-phenyl-1H-pyrazol-1-yl)-8-vinyl-9H-purin-6-yl)morpholine (0.9 g, 51%) as an yellow solid. LCMS (ESI) m/z: 432.0 [M+H]⁺.

### Step 3: Preparation of 1-(9-ethyl-2-(3-methoxy-4-phenyl-1H-pyrazol-1-yl)-6-morpholino-9H-purin-8-yl)ethane-1,2-diol

To a solution of 4-(9-ethyl-2-(3-methoxy-4-phenyl-1H-pyrazol-1-yl)-8-vinyl-9H-purin-6-yl)morpholine (0.7 g, 1.62 mmol) in acetone (15 mL) were added potassium osmate(VI) dihydrate (0.11 g, 0.32 mmol), 4-methylmorpholine N-oxide (280 mg, 2.43 mmol), 2-methylpropan-2-ol (4.5 mL) and water (4.5 mL). The resultant reaction mixture was stirred at 35° C. for 1 h under nitrogen, then filtered and the filtrate was concentrated. The resultant crude product was purified by silica gel column chromatography (dichloromethane:methanol=15:1) to give 1-(9-ethyl-2-(3-methoxy-4-phenyl-1H-pyrazol-1-yl)-6-morpholino-9H-purin-8-yl)ethane-1,2-diol (300 mg, 40%) as white solid. LCMS (ESI) m/z: 466.1 [M+H]⁺.

### Step 4: Preparation of 2-(9-ethyl-2-(3-methoxy-4-phenyl-1H-pyrazol-1-yl)-6-morpholino-9H-purin-8-yl)-2-methoxyethan-1-ol (Compound 55)) and 1-(9-ethyl-2-(3-methoxy-4-phenyl-1H-pyrazol-1-yl)-6-morpholino-9H-purin-8-yl)-2-methoxyethan-1-ol (Compound 56)

To a solution of 1-(9-ethyl-2-(3-methoxy-4-phenyl-1H-pyrazol-1-yl)-6-morpholino-9H-purin-8-yl)ethane-1,2-diol (300 mg, 0.64 mmol) in DMF (10 mL) were added iodomethane (100 mg, 0.71 mmol) and sodium hydride (52 mg, 1.3 mmol) at 0° C. and the resultant reaction mixture was stirred at 25° C. for 16 h. The entire mixture was then subjected to prep-HPLC (BOSTON pHlex ODS 10 μm 21.2×250 mm 120 A. The mobile phase was acetonitrile/0.1% ammonium bicarbonate) to give 2-(9-ethyl-2-(3-methoxy-4-phenyl-1H-pyrazol-1-yl)-6-morpholino-9H-purin-8-yl)-2-methoxyethan-1-ol (28.8 mg, 9%) and 1-(9-ethyl-2-(3-methoxy-4-phenyl-1H-pyrazol-1-yl)-6-morpholino-9H-purin-8-yl)-2-methoxyethan-1-ol (14.6 mg, 5% as white solids.

Compound 55: ¹H NMR (400 MHz, DMSO-d₆) δ 8.92 (s, 1H), 7.81 (d, J=7.2 Hz, 2H), 7.40 (t, J=7.7 Hz, 2H), 7.25 (t, J=7.4 Hz, 1H), 5.03 (bs, 1H), 4.63 (t, J=6.1 Hz, 1H), 4.45-4.20 (m, 6H), 4.07 (s, 3H), 3.91-3.81 (m, 2H), 3.80-3.74 (m, 4H), 3.32 (s, 3H), 1.40 (t, J=7.1 Hz, 3H). LCMS (ESI) m/z: 480.1 [M+H]⁺.

Compound 56: ¹H NMR (400 MHz, DMSO-d₆) δ 8.92 (s, 1H), 7.81 (d, J=7.3 Hz, 2H), 7.40 (t, J=7.7 Hz, 2H), 7.25 (t, J=7.3 Hz, 1H), 5.95 (s, 1H), 4.98 (s, 1H), 4.47-4.15 (m, 6H), 4.07 (s, 3H), 3.87-3.74 (m, 6H), 3.33 (s, 3H), 1.40 (t, J=7.1 Hz, 3H). LCMS (ESI) m/z: 480.1 [M+H]⁺.

### Synthesis of 2-(2-(3-(1-methyl-1H-pyrazol-3-yl)phenyl)-6-morpholino-9H-purin-9-yl)ethan-1-ol (Compound 395)

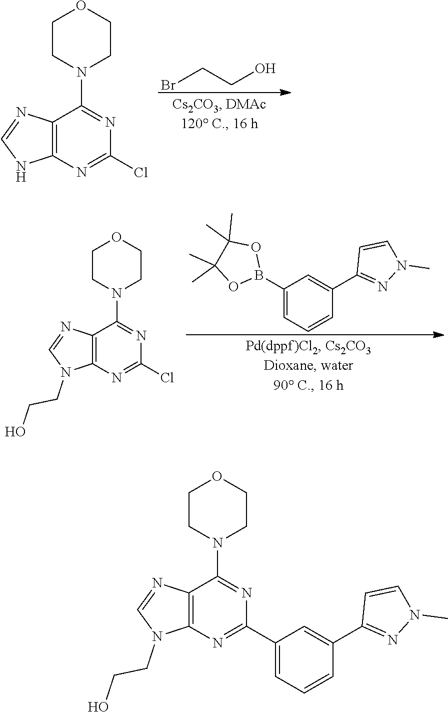

### Step 1: Synthesis of 2-(2-chloro-6-morpholino-9H-purin-9-yl)ethan-1-ol

To a solution of 4-(2-chloro-9H-purin-6-yl)morpholine (0.45 g, 1.88 mmol) in DMAc (30 mL) was added cesium carbonate (1.84 m, 5.65 mmol) followed by 2-bromoethan-1-ol (0.28 g, 2.26 mmol). The resulting mixture was stirred at 120° C. for 16 h. It was then diluted with water (40 mL) and extracted with dichloromethane (40 mL×2). The combined organic phase was washed with brine (40 mL), dried over sodium sulfate, filtered and concentrated. The resultant crude product was purified by flash chromatography (eluted with petroleum ether in ethyl acetate from 50% to 80%) to afford 2-(2-chloro-6-morpholino-9H-purin-9-yl)ethan-1-ol (0.33 g, 62.7%) as pale yellow oil. LCMS (ESI) m/z: 284.1 [M+H]⁺.

### Step 2: Preparation of 2-(2-(3-(1-methyl-1H-pyrazol-3-yl)phenyl)-6-morpholino-9H′purin-9-yl)ethan-1-ol

A mixture of 2-(2-chloro-6-morpholino-9H-purin-9-yl)ethan-1-ol (0.1 g, 0.35 mmol), 1-methyl-3-(3-(4,4,5,5-tetramethyl-1,3,2-dioxaborolan-2-yl)phenyl)-1H-pyrazole (0.12 g, 1.42 mmol), 1,1′-bis(diphenylphosphino)ferrocene-palladium(II)dichloride dichloromethane complex (30 mg, 0.04 mmol) and cesium carbonate (0.29 g, 0.88 mmol) in 1,4-dioxane/water (5 mL/1 mL) was stirred at 90° C. for 16 h under nitrogen atmosphere. The resultant reaction mixture was filtered and the filtrate was purified by prep-HPLC (base) to afford 2-(2-(3-(1-methyl-1H-pyrazol-3-yl)phenyl)-6-morpholino-9H-purin-9-yl) ethan-1-ol (11.2 mg, 7.9%) as yellow solid. ¹H NMR (400 MHz, DMSO-d₆) δ 8.75 (t, J=1.6 Hz, 1H), 8.32-8.29 (m, 1H), 8.18 (s, 1H), 7.87-7.82 (m, 1H), 7.78 (d, J=2.2 Hz, 1H), 7.49 (t, J=7.7 Hz, 1H), 6.74 (d, J=2.2 Hz, 1H), 5.05 (t, J=5.2 Hz, 1H), 4.40-4.25 (m, 6H), 3.92 (s, 3H), 3.83 (dd, J=10.5, 5.2 Hz, 2H), 3.80-3.75 (m, 4H). LCMS (ESI) m/z: 406.0 [M+H]⁺.

### Synthesis of 1-(9-ethyl-2-(3-(1-methyl-1H-pyrazol-3-yl)phenyl)-6-morpholino-9H-purin-8-yl)azetidin-3-ol (Compound 396)

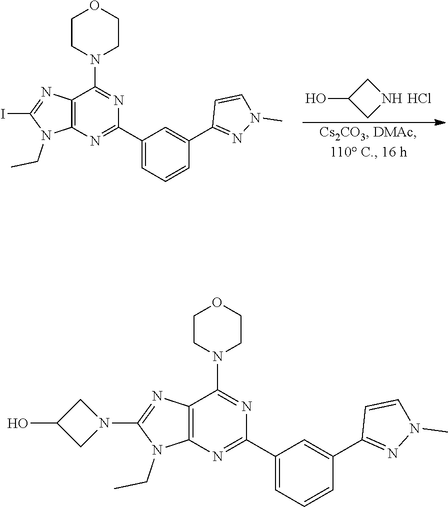 [SMILES: CCn1c(I)nc2c(N3CCOCC3)nc(-c3cccc(-c4ccn(C)n4)c3)nc21 ; OC1CNC1 ; Cl ; CCn1c(N2CC(O)C2)nc2c(N3CCOCC3)nc(-c3cccc(-c4ccn(C)n4)c3)nc21]

To a solution of 4-(9-ethyl-8-iodo-2-(3-(1-methyl-1H-pyrazol-3-yl)phenyl)-9H-purin-6-yl)morpholine (110 mg, 0.165 mmol) in N,N-dimethylacetamide (5 mL) were added azetidin-3-ol hydrochloride (52 mg, 0.47 mmol) and cesium carbonate (210 mg, 0.64 mmol). The mixture was stirred at 110° C. for 16 h and then filtered. The filtrate was subjected to prep-HPLC (BOSTON pHlex ODS 10 μm 21.2×250 mm 120 A, with mobile phase acetonitrile/0.1% ammonium bicarbonate) to obtain 1-(9-ethyl-2-(3-(1-methyl-1H-pyrazol-3-yl)phenyl)-6-morpholino-9H-purin-8-yl)azetidin-3-ol (6 mg, 6%) as a white solid. ¹HNMR (400 MHz, DMSO-d₆) δ 8.70 (s, 1H), 8.26 (d, J=7.9 Hz, 1H), 7.80 (d, J=8.0 Hz, 1H), 7.76 (d, J=2.1 Hz, 1H), 7.46 (t, J=7.7 Hz, 1H), 6.72 (d, J=2.2 Hz, 1H), 5.74 (d, J=6.6 Hz, 1H), 4.61 (pent, J=5.4 Hz, 1H), 4.35 (t, J=7.5 Hz, 2H), 4.20 (bs, 4H), 4.08 (quart, J=7.1 Hz, 2H), 3.95 (dd, J=8.4, 5.2 Hz, 2H), 3.92 (s, 3H), 3.82-3.72 (m, 4H), 1.34 (t, J=7.1 Hz, 3H). LCMS (ESI) m/z: 461.3 [M+H]⁺.

### Synthesis of Preparation of 4-(9-ethyl-2-(3-methoxy-4-phenyl-1H-pyrazol-1-yl)-8-(methyl-d₃)-9H-purin-6-yl)morpholine (Compound 397)

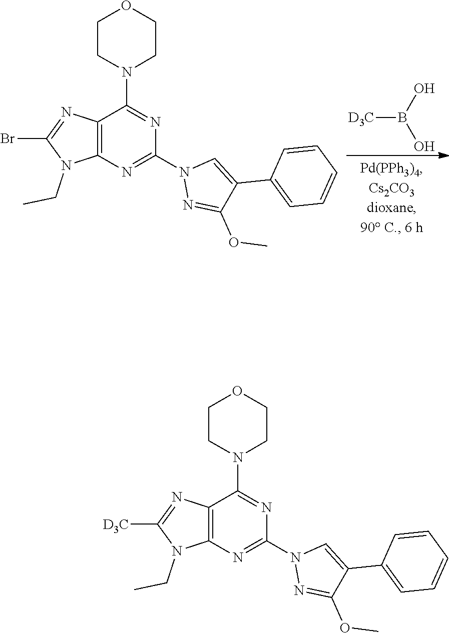

A solution of 4-(8-bromo-9-ethyl-2-(3-methoxy-4-phenyl-1H-pyrazol-1-yl)-9H-purin-6-yl)morpholine (60 mg, 0.124 mmol), (methyl-d₃)boronic acid (8 mg, 0.162 mmol), tetrakis(triphenylphosphine)palladium (10 mg, 0.01 mmol) and cesium carbonate (68 mg, 0.3 mmol) in 1,4-dioxane (5 mL) was stirred at 90° C. for 6 h under nitrogen atmosphere. The mixture was then filtered, concentrated and purified by prep-HPLC (BOSTON pHlex ODS 10 μm 21.2×250 mm 120 A. The mobile phase was acetonitrile/0.1% ammonium bicarbonate) to obtain 4-(9-ethyl-2-(3-methoxy-4-phenyl-1H-pyrazol-1-yl)-8-(methyl-d₃)-9H-purin-6-yl)morpholine (18.7 mg, 35.72%) as white solid. ¹H NMR (400 MHz, DMSO-d₆) δ 8.91 (s, 1H), 7.81 (d, J=7.2 Hz, 2H), 7.39 (t, J=7.7 Hz, 2H), 7.24 (t, J=7.4 Hz, 1H), 4.28 (bs, 4H), 4.20 (q, J=7.2 Hz, 2H), 4.07 (s, 3H), 3.78-3.74 (m, 4H), 1.34 (t, J=7.2 Hz, 3H). LCMS (ESI) m/z: 423.1 [M+H]⁺.

### Synthesis of 9-ethyl-2-(3-(1-methyl-1H-pyrazol-3-yl)phenyl)-6-(pyrimidin-4-yl)-9H-purine (Compound 398)

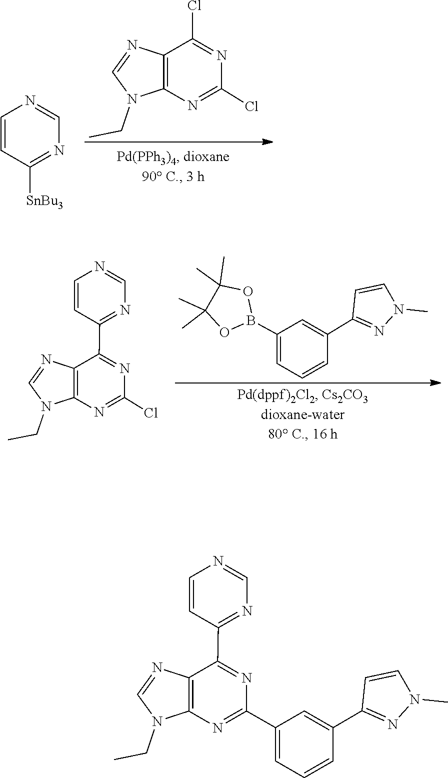

### Step 1: Synthesis of 2-chloro-9-ethyl-6-(pyrimidin-4-yl)-9H-purine

A mixture of 2,6-dichloro-9-ethyl-9H-purine (100 mg, 0.46 mmol), 4-(tributylstannyl)pyrimidine (169 mg, 0.46 mmol) and tetrakis(triphenylphosphine)palladium (53 mg 0.046 mmol) in 1,4-dioxane (3 mL) was stirred at 100° C. for 16 h under argon atmosphere. The residue was partitioned between ethyl acetate (50 mL) and water (50 mL), the organic layer was washed with brine, dried and evaporated to dryness. The resultant crude product was purified by flash chromatography on silica gel (petroleum ether/ethyl acetate 20:1→10:1) to give 2-chloro-9-ethyl-6-(pyrimidin-4-yl)-9H-purine (30 mg, 25%) as a white solid. LCMS (ESI) m/z: 261.0 [M+H]⁺.

### Step 2: Synthesis of 9-ethyl-2-(3-(1-methyl-1H-pyrazol-3-yl)phenyl)-6′(pyrimidin-4-yl)-9H-purine

A mixture of 2-chloro-9-ethyl-6-(pyrimidin-4-yl)-9H-purine (30 mg, 0.12 mmol), 1-methyl-3-(3-(4,4,5,5-tetramethyl-1,3,2-dioxaborolan-2-yl)phenyl)-1H-pyrazole (49 mg, 0.17 mmol), [1,1′-bis(diphenylphosphino)ferrocene]dichloropalladium(II) (9 mg, 0.012 mL) and potassium carbonate (33 mg, 0.24 mmol) in 1,4-dioxane (1 mL) and water (0.1 mL) was stirred at 80° C. for 16 h under argon atmosphere.

The resultant mixture was partitioned between ethyl acetate (50 mL) and water (50 mL), the organic layer was washed with brine, dried and evaporated to dryness. The crude product was purified by flash chromatography on silica gel (petroleum ether/ethyl acetate 10:1→1:1) to give the 9-ethyl-2-(3-(1-methyl-1H-pyrazol-3-yl)phenyl)-6-(pyrimidin-4-yl)-9H-purine (26.7 mg, 67%) as a white solid. ¹H NMR (400 MHz, DMSO-d₆) δ 9.52 (s, 1H), 9.15 (d, J=5.1 Hz, 1H), 8.97 (s, 1H), 8.89-8.71 (m, 2H), 8.52 (d, J=7.8 Hz, 1H), 7.93 (d, J=7.7 Hz, 1H), 7.80 (d, J=1.9 Hz, 1H), 7.60 (t, J=7.7 Hz, 1H), 6.80 (d, J=2.1 Hz, 1H), 4.47 (q, J=7.2 Hz, 2H), 3.95 (s, 3H), 1.59 (t, J=7.3 Hz, 3H). LCMS (ESI) m/z: 383.2 [M+H]⁺.

### Synthesis of Preparation of 4-(9-ethyl-2-(3-methoxy-4-phenyl-1H-pyrazol-1-yl)-8-(pyridin-3-yl)-9H-purin-6-yl)morpholine (Compound 399)

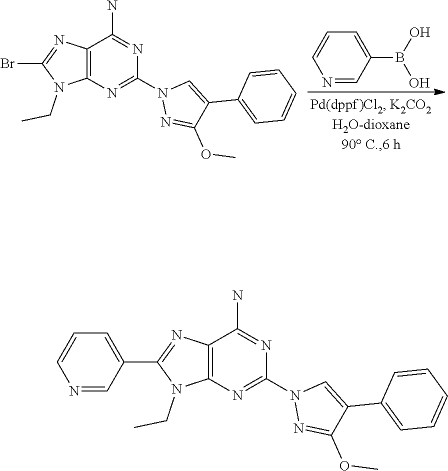

A solution of 4-(8-bromo-9-ethyl-2-(3-methoxy-4-phenyl-1H-pyrazol-1-yl)-9H-purin-6-yl)morpholine (60 mg, 0.0124 mmol), pyridin-3-ylboronic acid (18 mg, 0.146 mmol), 1,1′-bis(diphenylphosphino)ferrocene-palladium(II)dichloride dichloromethane complex (12 mg, 0.014 mmol), and potassium carbonate (69 mg, 0.5 mmol) in 1,4-dioxane/water (10 mL/1 mL) was stirred at 90° C. for 6 h under nitrogen atmosphere. The mixture was filtered, concentrated and purified by prep-HPLC (BOSTON pHlex ODS 10 μm 21.2×250 mm 120 A. The mobile phase was acetonitrile/0.1% ammonium bicarbonate) to obtain 4-(9-ethyl-2-(3-methoxy-4-phenyl-1H-pyrazol-1-yl)-8-(pyridin-3-yl)-9H-purin-6-yl)morpholine (34.4 mg, 43.12%) as white solid. ¹H NMR (400 MHz, DMSO-d₆) δ 9.02 (d, J=1.6 Hz, 1H), 8.98 (s, 1H), 8.77 (dd, J=4.8, 1.6 Hz, 1H), 8.27-8.23 (m, 1H), 7.83 (d, J=7.2 Hz, 2H), 7.63 (dd, J=7.6, 4.6 Hz, 1H), 7.41 (t, J=7.7 Hz, 2H), 7.26 (t, J=7.4 Hz, 1H), 4.50-4.25 (m, 6H), 4.09 (s, 3H), 3.81-3.77 (m, 4H), 1.30 (t, J=7.2 Hz, 3H). LCMS (ESI) m/z: 483.1 [M+H]⁺.

### Synthesis of (9-ethyl-2-(3-methoxy-4-phenyl-1H-pyrazol-1-yl)-6-morpholino-9H-purin-8-yl)(imino)(methyl)-16-sulfanone (Compound 400)

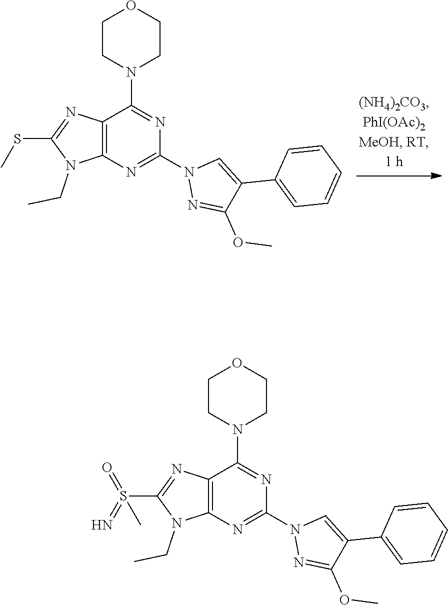 [SMILES: CCn1c(SC)nc2c(N3CCOCC3)nc(-n3cc(-c4ccccc4)c(OC)n3)nc21 ; CCn1c(S(C)(=N)=O)nc2c(N3CCOCC3)nc(-n3cc(-c4ccccc4)c(OC)n3)nc21]

A solution of 4-(9-ethyl-2-(3-methoxy-4-phenyl-1H-pyrazol-1-yl)-8-(methylthio)-9H-purin-6-yl)morpholine (173 mg, 0.384 mmol), (diacetoxyiodo)benzene (55 mg, 0.576 mmol) and ammonium carbonate (284 mg, 0.8832 mmol) in methanol (10 mL) was stirred at room temperature for 1 h under nitrogen atmosphere. The reaction mixture was filtered, concentrated and purified by prep-HPLC (BOSTON pHlex ODS 10 μm 21.2×250 mm 120 A. The mobile phase was acetonitrile/0.1% ammonium bicarbonate) to obtain (9-ethyl-2-(3-methoxy-4-phenyl-1H-pyrazol-1-yl)-6-morpholino-9H-purin-8-yl)(imino)(methyl)-16-sulfanone (67.7 mg 36.71%) as white solid. ¹H NMR (400 MHz, DMSO-d₆) δ 8.97 (s, 1H), 7.82 (d, J=7.2 Hz, 2H), 7.41 (t, J=7.7 Hz, 2H), 7.26 (t, J=7.4 Hz, 1H), 5.33 (s, 1H), 4.60-4.25 (m, 6H), 4.08 (s, 3H), 3.81-3.75 (m, 4H), 3.39 (s, 3H), 1.44 (t, J=7.1 Hz, 3H). LCMS (ESI) m/z: 483.1 [M+H]⁺.

### Synthesis of 2-(9-ethyl-6-morpholino-9H-purin-2-yl)-2,5-dihydroisochromeno[3,4-c]pyrazole (Compound 401)

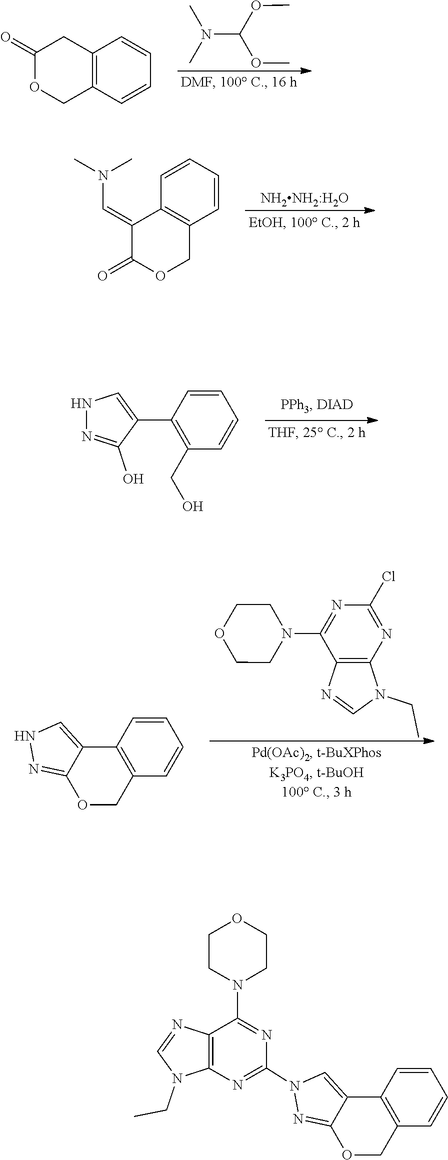

### Step 1: Synthesis of (E)-4-((dimethylamino)methylene)isochroman-3-one

A mixture of isochroman-3-one (2.0 g, 13.5 mmol) and 1,1-dimethoxy-N,N-dimethylmethanamine (8.04 g, 67.5 mmol) in N,N-dimethylformamide (30 mL) was stirred at 110° C. for 16 h. To the reaction mixture water (25 mL) was added and extracted with ethyl acetate (25 mL*3). The combined organic phase was concentrated to give (E)-4-((dimethylamino)methylene)isochroman-3-one (2 g, 73%) as a yellow oil. LCMS (ESI) m/z: 204.1 [M+H]⁺.

### Step 2: Synthesis of 4-(2-(hydroxymethyl)phenyl)-1H-pyrazol-3-ol

A mixture of (E)-4-((dimethylamino)methylene)isochroman-3-one (3.0 g, 14.76 mmol) and hydrazine hydrate (3.7 g, 73.8 mmol) in ethanol (40 mL) was stirred at 100° C. for 2 h. To the resultant mixture water (25 mL) was added and extracted with ethyl acetate (25 mL*3). The combined organic phase was concentrated to obtain 4-(2-(hydroxymethyl)phenyl)-1H-pyrazol-3-ol (2 g, 71%) as a yellow oil. LCMS (ESI) m/z: 191.1 [M+H]⁺.

### Step 3: Synthesis of 2,5-dihydroisochromeno[3,4-c]pyrazole

To a solution of 4-(2-(hydroxymethyl)phenyl)-1H-pyrazol-3-ol (0.5 g, 2.6 mmol) in tetrahydrofuran (16 mL) were added triphenylphosphine (0.97 g, 3.7 mmol) and diisopropylazodicarboxylate (0.7 g, 3.4 mmol) at 0° C. and the resultant reaction mixture was stirred at 20° C. for 2 h. It was then concentrated and purified by silica gel column (petroleum ether:ethyl acetate=2:1) to obtain 2,5-dihydroisochromeno[3,4-c]pyrazole (0.18 g, 40%) as off-white solid. LCMS (ESI) m/z: 173.1 [M+H]⁺.

### Step 4: Synthesis of 2-(9-ethyl-6-morpholino-9H-purin-2-yl)-2,5-dihydroiso chromeno[3,4-c]pyrazole

To a mixture of 2,5-dihydroisochromeno[3,4-c]pyrazole (0.07 g, 0.41 mmol) in tert-butanol (3 mL) were added 4-(2-chloro-9-ethyl-9H-purin-6-yl)morpholine (0.1 g, 0.37 mmol), potassium phosphate tribasic (0.16 g, 0.75 mmol), palladium(II)acetate (0.017 g, 0.07 mmol) and 2-di-t-butylphosphino-2′,4′,6′-tri-i-propyl-1,1′-biphenyl (0.03 g, 0.07 mmol). The resultant reaction mixture was stirred at 100° C. for 3 h under argon atmosphere. The mixture was then filtered, the filtrate was diluted with DMF and the mixture was subjected to prep-HPLC (BOSTON pHlex ODS 10 μm 21.2×250 mm 120 A, with mobile phase acetonitrile/0.1% ammonium bicarbonate) to obtain 2-(9-ethyl-6-morpholino-9H-purin-2-yl)-2,5-dihydroisochromeno[3,4-c]pyrazole as an off-white solid (15.4 mg, 10%). ¹H NMR (400 MHz, DMSO-d₆) δ 8.98 (s, 1H), 8.20 (s, 1H), 7.74 (d, J=7.5 Hz, 1H), 7.36 (dd, J=9.9, 4.3 Hz, 1H), 7.27 (t, J=7.0 Hz, 2H), 5.37 (s, 2H), 4.27 (bs, 4H), 4.22 (q, J=7.3 Hz, 2H), 3.83-3.69 (m, 4H), 1.44 (t, J=7.3 Hz, 3H). LCMS (ESI) m/z: 404.1 [M+H]⁺.

### Synthesis of 6-(9-ethyl-2-(3-methoxy-4-phenyl-1H-pyrazol-1-yl)-9H-purin-6-yl)-2-methylpyridazin-3(2H)-one (Compound 402)

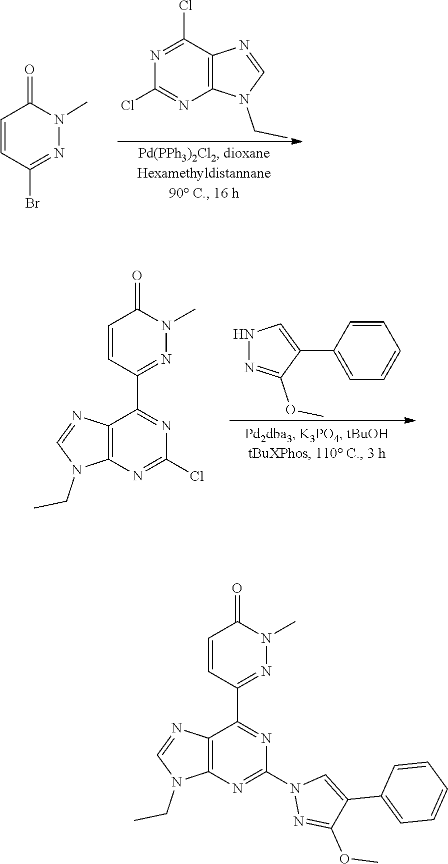

### Step 1: Synthesis of 6-(2-chloro-9-ethyl-9H-purin-6-yl)-2-methylpyridazin-3(2H)-one

A mixture of 6-bromo-2-methylpyridazin-3(2H)-one (200 mg, 1.05 mmol), 2,6-dichloro-9-ethyl-9H-purine (298 mg, 1.37 mmol), bis(triphenylphosphine)palladium(II) chloride (70 mg, 0.105 mmol), and hexamethyldistannane (343 mg, 1.05 mmol) in dioxane (3 mL) was stirred at 90° C. under argon for 16 h. The resultant mixture was then filtered, and the filtrate was concentrated. The crude product was chromatographed on silica gel (dichloromethane/methanol 50:1→20:1) to obtain 6-(2-chloro-9-ethyl-9H-purin-6-yl)-2-methylpyridazin-3(2H)-one (50 mg, 17%) as a white solid. LCMS (ESI) m/z: 291.1 [M+H]⁺

### Step 2: Synthesis of 6-(9-ethyl-2-(3-methoxy-4-phenyl-1H-pyrazol-1-yl)-9H-purin-6-yl)-2-methylpyridazin-3(2H)-one

A mixture of 6-(2-chloro-9-ethyl-9H-purin-6-yl)-2-methylpyridazin-3(2H)-one (50 mg, 0.17 mmol), 3-methoxy-4-phenyl-1H-pyrazole (45 mg, 0.25 mmol), potassium phosphate tribasic (108 mg, 0.51 mmol), tris(dibenzylideneacetone)dipalladium (15 mg, 0.017 mmol) and 2-di-tert-butylphosphino-2′,4′,6′-trisopropylbinphenyl (14 mL, 0.034 mmol) in tert-butanol (3 mL) was stirred at 110° C. under argon for 3 h.

The resultant mixture was filtered, and the filtrate was concentrated. The crude product was chromatographed on silica gel (dichloromethane/methanol 20:1→10:1) to obtain 6-(9-ethyl-2-(3-methoxy-4-phenyl-1H-pyrazol-1-yl)-9H-purin-6-yl)-2-methylpyridazin-3(2H)-one (9.3 mg, 13%) as a white solid. ¹H NMR (500 MHz, DMSO-d₆) δ 9.14 (s, 1H), 8.73 (d, J=9.7 Hz, 1H), 8.69 (s, 1H), 7.86 (d, J=7.5 Hz, 2H), 7.43 (t, J=7.7 Hz, 2H), 7.28 (s, 1H), 7.17 (d, J=9.8 Hz, 1H), 4.37 (q, J=7.3 Hz, 2H), 4.12 (s, 3H), 3.87 (s, 3H), 1.53 (t, J=7.3 Hz, 3H); LCMS (ESI) m/z: 429.0 [M+H]⁺.

### Synthesis of 4-(9-ethyl-2-(3-methoxy-4-phenyl-1H-pyrazol-1-yl)-8-(methylsulfinyl)-9H-purin-6-yl)morpholine (Compound 403)

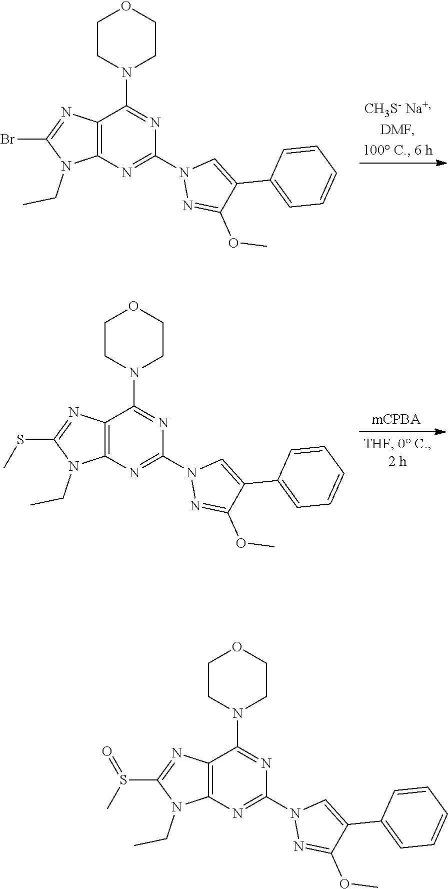 [SMILES: CCn1c(Br)nc2c(N3CCOCC3)nc(-n3cc(-c4ccccc4)c(OC)n3)nc21 ; CCn1c(SC)nc2c(N3CCOCC3)nc(-n3cc(-c4ccccc4)c(OC)n3)nc21 ; CCn1c(S(C)=O)nc2c(N3CCOCC3)nc(-n3cc(-c4ccccc4)c(OC)n3)nc21]

### Step 1: Synthesis of 4-(9-ethyl-2-(3-methoxy-4-phenyl-1H-pyrazol-1-yl)-8-(methylthio)-9H-purin-6-yl)morpholine

A solution of 4-(8-bromo-9-ethyl-2-(3-methoxy-4-phenyl-1H-pyrazol-1-yl)-9H-purin-6-yl)morpholine (350 mg, 0.725 mmol), sodium thiomethoxide (153 mg, 2.175 mmol) in N,N-dimethylformamide (8 mL) was stirred at 100° C. for 6 h under nitrogen atmosphere. The resultant mixture was filtered, concentrated and purified by silica gel chromatography eluting with a linear gradient of 0% to 36% ethyl acetate in petroleum ether to afford 4-(9-ethyl-2-(3-methoxy-4-phenyl-1H-pyrazol-1-yl)-8-(methylthio)-9H-purin-6-yl)morpholine (296 mg, 90.48%) as white solid. LCMS (ESI) m/z: 452.0 [M+H]⁺.

### Step 2: Synthesis of 4-(9-ethyl-2-(3-methoxy-4-phenyl-1H-pyrazol-1-yl)-8-(methylsulfinyl)-9H-purin-6-yl)morpholine

A solution of 4-(9-ethyl-2-(3-methoxy-4-phenyl-1H-pyrazol-1-yl)-8-(methylthio)-9H-purin-6-yl)morpholine (80 mg, 0.177 mmol) and 3-chloroperoxybenzoic acid (16 mg, 0.088 mmol) in tetrahydrofuran (5 mL) was stirred at 0° C. for 2 h under nitrogen atmosphere. The resultant mixture was filtered, concentrated and purified by prep-HPLC (BOSTON pHlex ODS 10 μm 21.2×250 mm 120 A). The mobile phase was acetonitrile/0.1% Ammonium bicarbonate) to obtain 4-(9-ethyl-2-(3-methoxy-4-phenyl-1H-pyrazol-1-yl)-8-(methylsulfinyl)-9H-purin-6-yl)morpholine (25.7 mg, 31.07%) as white solid. ¹H NMR (400 MHz, DMSO-d₆) δ 8.97 (s, 1H), 7.84-7.81 (m, 2H), 7.40 (t, J=7.7 Hz, 2H), 7.26 (t, J=7.4 Hz, 1H), 4.87-4.19 (m, 6H), 4.08 (s, 3H), 3.79 (t, J=4.7 Hz, 4H), 3.18 (s, 3H), 1.47 (t, J=7.2 Hz, 3H) LCMS (ESI) m/z: 468.1 [M+H]⁺.

### Synthesis of 2-(3-methoxy-4-phenyl-1H-pyrazol-1-yl)-4-morpholinopyrido[3′,2′:4,5]furo[3,2-d]pyrimidine (Compound 404)

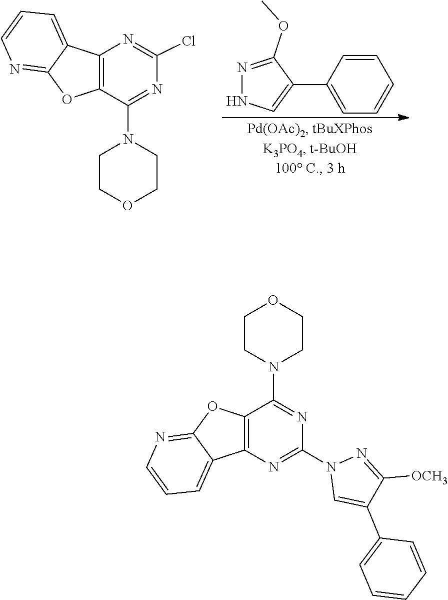 [SMILES: Clc1nc(N2CCOCC2)c2oc3ncccc3c2n1 ; COc1n[nH]cc1-c1ccccc1 ; COc1nn(-c2nc(N3CCOCC3)c3oc4ncccc4c3n2)cc1-c1ccccc1]

To a mixture of 3-methoxy-4-phenyl-1H-pyrazole (0.05 g, 0.29 mmol) in tert-butyl alcohol (3.5 mL) were added 2-chloro-4-morpholinopyrido[3′,2′:4,5]furo[3,2-d]pyrimidine (0.06 g, 0.21 mmol), potassium phosphate tribasic (0.09 g, 0.41 mmol), palladium(II)acetate (0.009 g, 0.04 mmol) and 2-di-t-butylphosphino-2′,4′,6′-tri-i-propyl-1,1′-biphenyl (0.017 g, 0.04 mmol). The resultant mixture was stirred at 100° C. for 3 h under argon atmosphere. It was filtered, concentrated, redissolved with N,N-dimethylformamide and subjected to prep-HPLC (BOSTON pHlex ODS 10 μm 21.2×250 mm 120 A. The mobile phase was acetonitrile/0.1% Ammonium bicarbonate) to obtain 2-(3-methoxy-4-phenyl-1H-pyrazol-1-yl)-4-morpholinopyrido[3′,2′:4,5]furo[3,2-d]pyrimidine as an off-white solid (4.6 mg, 5%). ¹H NMR (500 MHz, DMSO-d₆) δ 8.98 (s, 1H), 8.70-8.66 (m, 2H), 7.84 (d, J=7.3 Hz, 2H), 7.64 (dd, J=7.6, 4.8 Hz, 1H), 7.42 (t, J=7.7 Hz, 2H), 7.27 (t, J=7.4 Hz, 1H), 4.16 (d, J=4.6 Hz, 4H), 4.11 (s, 3H), 3.88-3.83 (m, 4H); LCMS (ESI) m/z: 429.1 [M+H]⁺.

### Synthesis of 2-(9-ethyl-6-morpholino-9H-purin-2-yl)isochromeno[3,4-c]pyrazol-5(2H)-one (Compound 405)

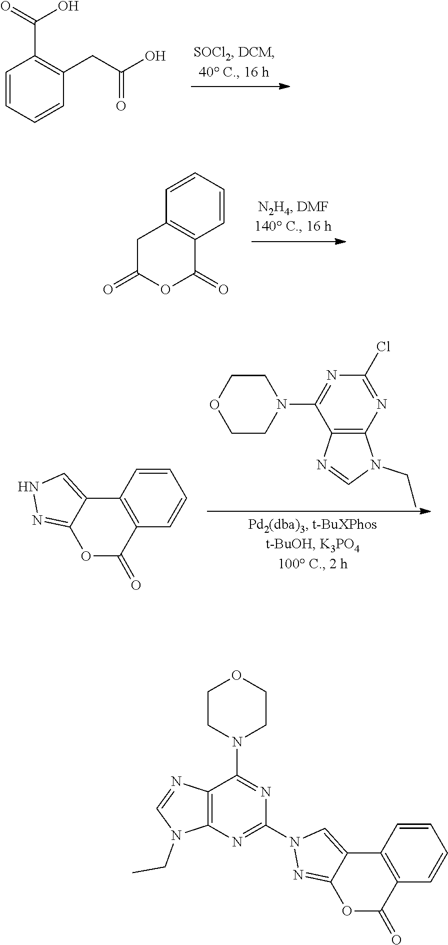 [SMILES: O=C(O)Cc1ccccc1C(=O)O ; O=C1Cc2ccccc2C(=O)O1 ; O=c1oc2n[nH]cc2c2ccccc12 ; CCn1cnc2c(N3CCOCC3)nc(Cl)nc21 ; CCn1cnc2c(N3CCOCC3)nc(-n3cc4c(n3)oc(=O)c3ccccc34)nc21]

### Step 1: Synthesis of isochromane-1,3-dione

Homophthalic acid (3 g, 0.017 mol) was dissolved in anhydrous dichloromethane (50 mL) and thionyl chloride was then added (4.83 mL, 0.067 mol) dropwise. The mixture was then stirred at 40° C. for 16 h. The resultant mixture was then concentrated and the residue obtained was dried under high vacuum to remove remaining trace amounts of thionyl chloride to give isochromane-1,3-dione (1.8 g, 67%) as yellow solid. LCMS (ESI) m/z: 163.1 [M+H]⁺.

### Step 2: Synthesis of isochromeno[3,4-c]pyrazol-5(2H)-one

The isochromane-1,3-dione from step 1 (1.8 g, 11.1 mmol) was dissolved in anhydrous DMF (50 mL) and stirred for 15 minutes at room temperature. Hydrazine monohydrate (2.2 g, 44.4 mmol) was then added dropwise and the mixture heated to 140° C. and stirred for 16 h. The resultant mixture was cooled and water was added. The precipitate obtained was then filtered, washed with water, and dried to obtain isochromeno[3,4-c]pyrazol-5(2H)-one (0.11 g, 5%). LCMS (ESI) m/z: 187.1 [M+H]⁺.

### Step 3: Synthesis of 2-(9-ethyl-6-morpholino-9H-purin-2-yl)isochromeno[3,4-c]pyrazol-5(2H)-one

A mixture of 4-(2-chloro-9-ethyl-9H-purin-6-yl)morpholine (0.12 g, 0.45 mmol), isochromeno[3,4-c]pyrazol-5(2H)-one (0.083 g, 0.45 mmol), potassium phosphate tribasic (0.19 g, 0.9 mmol), tris(dibenzylideneacetone)dipalladium(0) (0.045 g, 0.05 mmol), and 2-di-t-butylphosphino-2′,4′,6′-tri-i-propyl-1,1′-biphenyl (0.043 g, 0.1 mmol) in tert-butyl alcohol (3.5 mL) was stirred at 100° C. for 2 h. The resultant reaction mixture was concentrated, and the crude product thus obtained was subjected to prep-HPLC (BOSTON pHlex ODS 10 μm 21.2×250 mm 120 A. The mobile phase was acetonitrile/0.1% formic acid) to obtain the desired product as an off-white solid (4.0 mg, 2%). ¹H NMR (400 MHz, DMSO-d₆) δ 9.42 (s, 1H), 8.26 (s, 1H), 8.22 (s, 1H), 8.20 (s, 1H), 7.94-7.87 (m, 1H), 7.57 (dd, J=11.6, 4.6 Hz, 1H), 4.60-4.20 (m, 4H), 4.25 (q, J=7.3 Hz, 2H), 3.83-3.75 (m, 4H), 1.46 (t, J=7.3 Hz, 3H). LCMS (ESI) m/z: 418.1 [M+H]⁺.

### Synthesis of 5-(9-ethyl-2-(3-methoxy-4-phenyl-1H-pyrazol-1-yl)-9H-purin-6-yl)-1-methylpyridin-2(1H)-one (Compound 406)

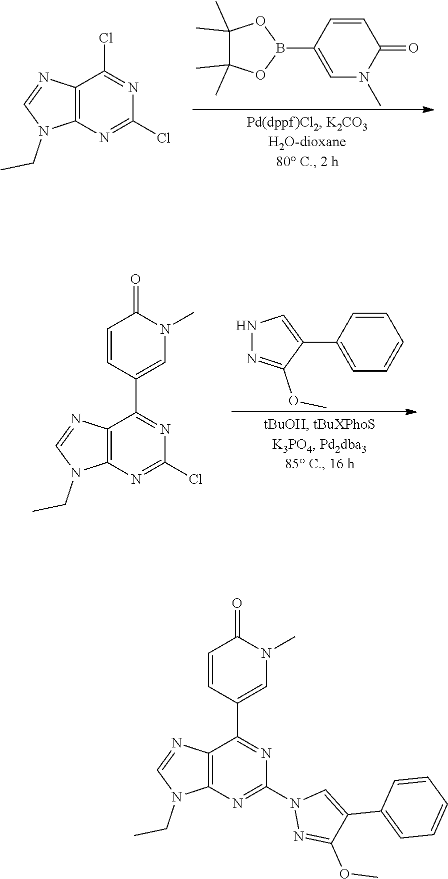 [SMILES: CCn1cnc2c(Cl)nc(Cl)nc21 ; Cn1cc(B2OC(C)(C)C(C)(C)O2)ccc1=O ; CCn1cnc2c(-c3ccc(=O)n(C)c3)nc(Cl)nc21 ; COc1n[nH]cc1-c1ccccc1 ; CCn1cnc2c(-c3ccc(=O)n(C)c3)nc(-n3cc(-c4ccccc4)c(OC)n3)nc21]

### Step 1: Synthesis of 5-(2-chloro-9-ethyl-9H-purin-6-yl)-1-methylpyridin-2(1H)-one

A mixture of 2,6-dichloro-9-ethyl-9H-purine (300 mg, 1.38 mmol), 1-methyl-5-(4,4,5,5-tetramethyl-1,3,2-dioxaborolan-2-yl)pyridin-2(1H)-one (308 mg, 1.31 mmol), [1,1′-bis(diphenylphosphino)ferrocene]dichloropalladium(II) (100 mg, 0.14 mmol) and potassium carbonate (517 mgmg, 4.14 mmol) in 1,4-dioxane (5 mL) and water (0.5 mL) was stirred at 80° C. under argon atmosphere for 2 h. The resultant reaction mixture was filtered, and the filtrate was concentrated. The crude product thus obtained was purified by column chromatography on silica gel (eluted with dichloromethane/methanol 20:1→10:1) to obtain 5-(2-chloro-9-ethyl-9H-purin-6-yl)-1-methylpyridin-2(1H)-one (200 mg, 50%) as white solid. LCMS (ESI) m/z: 290.0 [M+H]⁺.

### Step 2: Synthesis of 5-(9-ethyl-2-(3-methoxy-4-phenyl-1H-pyrazol-1-yl)-9H-purin-6-yl)-1-methylpyridin-2(1H)-one

A mixture of 5-(2-chloro-9-ethyl-9H-purin-6-yl)-1-methylpyridin-2(1H)-one (150 mg, 0.52 mmol), 3-methoxy-4-phenyl-1H-pyrazole (135 mg, 0.78 mmol), tris(dibenzylideneacetone)dipalladium (47 mg, 0.052 mmol), potassium phosphate tribasic (330 mg, 1.56 mmol) and 2-di-tert-butylphosphino-2′,4′,6′-trisopropylbinphenyl (44 mg, 0.104 mmol) in tert-butanol (3 mL) was stirred at 85° C. under argon for 16 h. The resultant mixture was filtered, and the filtrate was concentrated. The crude product thus obtained was purified by column chromatography on silica gel (dichloromethane/methanol 20:1→10:1) to obtain 5-(9-ethyl-2-(3-methoxy-4-phenyl-1H-pyrazol-1-yl)-9H-purin-6-yl)-1-methylpyridin-2(1H)-one (89.5 mg, 40%) as white solid. ¹H NMR (400 MHz, DMSO-d₆) δ 9.45 (s, 1H), 9.24 (s, 1H), 9.00 (d, J=9.5 Hz, 1H), 8.62 (s, 1H), 7.88 (d, J=7.5 Hz, 2H), 7.43 (t, J=7.6 Hz, 2H), 7.28 (t, J=7.3 Hz, 1H), 6.63 (d, J=9.6 Hz, 1H), 4.34 (q, J=7.2 Hz, 2H), 4.12 (s, 3H), 3.66 (s, 3H), 1.53 (t, J=7.3 Hz, 3H). LCMS (ESI) m/z: 428.1 [M+H]⁺.

### Synthesis of (9-ethyl-2-(3-methoxy-4-phenyl-1H-pyrazol-1-yl)-6-morpholino-9H-purin-8-yl)dimethyl-phosphine oxide (Compound 407)

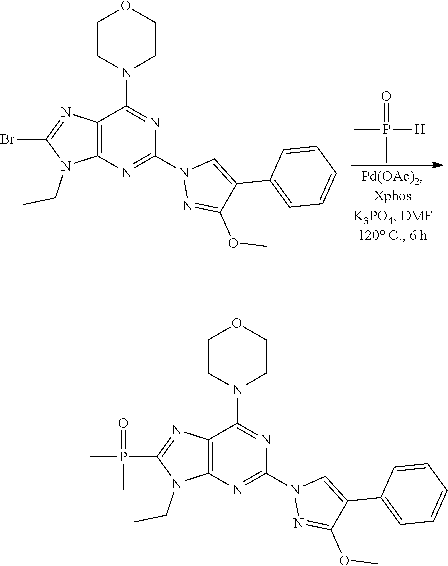

A solution of 4-(8-bromo-9-ethyl-2-(3-methoxy-4-phenyl-1H-pyrazol-1-yl)-9H-purin-6-yl)morpholine (140 mg, 0.29 mmol), dimethylphosphine oxide (34 mg, 0.435 mmol), palladium (II) acetate (7 mg, 0.03 mmol), 2-(dicyclohexylphosphino)-2′,4′,6′-triisopropylbiphenyl (14 mg, 0.03 mmol) and potassium phosphate tribasic (153 mg, 0.725 mmol) in N,N-dimethylformamide (5 mL) was stirred at 120° C. for 6 h under nitrogen atmosphere. The reaction mixture was filtered, concentrated and purified by prep-HPLC (BOSTON pHlex ODS 10 μm 21.2×250 mm 120 A. The mobile phase was acetonitrile/0.1% ammonium bicarbonate) to obtain (9-ethyl-2-(3-methoxy-4-phenyl-1H-pyrazol-1-yl)-6-morpholino-9H-purin-8-yl)dimethylphosphine oxide (14.3 mg, 10.27%) as white solid. ¹H NMR (400 MHz, CDCl₃) δ 8.71 (s, 1H), 7.89 (dd, J=8.4, 2.5 Hz, 2H), 7.78-7.71 (m, 3H), 4.92-4.23 (m, 6H), 4.22 (s, 3H), 3.93-3.87 (m, 4H), 1.77 (s, 3H), 1.74 (s, 3H), 1.58 (t, J=7.4 Hz, 3H). LCMS (ESI) m/z: 482.1 [M+H]⁺.

### Synthesis of 4-(9-ethyl-2-(3-methoxy-4-(4-(trifluoromethyl)pyridin-2-yl)-1H-pyrazol-1-yl)-9H-purin-6-yl)morpholine (Compound 408)

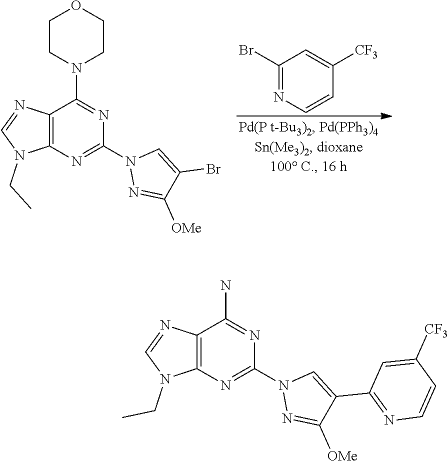

A mixture of 4-(2-(4-bromo-3-methoxy-1H-pyrazol-1-yl)-9-ethyl-9H-purin-6-yl)morpholine (100 mg, 0.247 mmol), 2-bromo-4-(trifluoromethyl)pyridine (67 mg, 0.296 mmol), bis(tri-tert-butylphosphine)palladium(0) (35 mg, 0.05 mmol), tetrakis(triphenyl phosphine)palladium (30 mg, 0.025 mmol), and hexamethyldistannane (125 mg, 0.37 mmol) in 1,4-dioxane (5 mL) was stirred at 90° C. for 16 h under nitrogen atmosphere. The mixture was then filtered, concentrated and the residue obtained was purified by prep-HPLC (BOSTON pHlex ODS 10 μm 21.2×250 mm 120 A. The mobile phase was acetonitrile/0.1% ammonium bicarbonate) to obtain 4-(9-ethyl-2-(3-methoxy-4-(4-(trifluoromethyl)pyridin-2-yl)-1H-pyrazol-1-yl)-9H-purin-6-yl)morpholine (36.6 mg, 31.25%) as white solid. ¹H NMR (400 MHz, DMSO-d₆) δ 9.06 (s, 1H), 8.87 (d, J=5.1 Hz, 1H), 8.25 (s, 1H), 8.04 (s, 1H), 7.62 (d, J=5.1 Hz, 1H), 4.60-4.25 (m, 4H), 4.23 (q, J=7.2 Hz, 2H), 4.14 (s, 3H), 3.80-3.76 (m, 4H), 1.46 (t, J=7.3 Hz, 3H). LCMS (ESI) m/z: 475.0 [M+H]⁺.

### Synthesis of (S)-(4-(9-ethyl-2-(3-methoxy-4-phenyl-1H-pyrazol-1-yl)-9H-purin-6-yl)morpholin-3-yl)methanol (Compound 409)

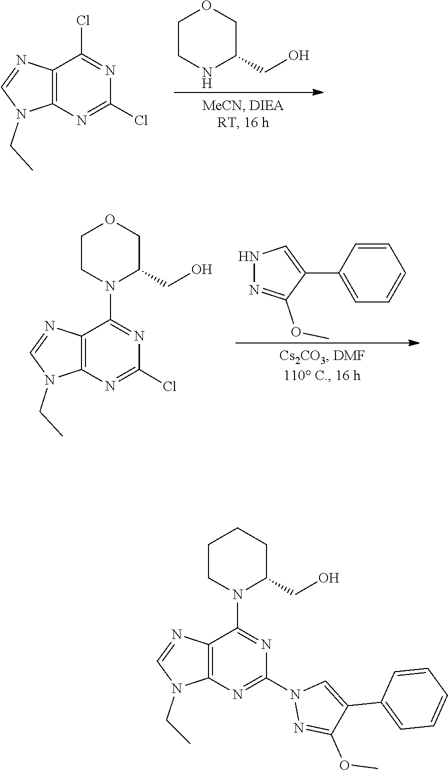

### Step 1: Synthesis of (S)-(4-(2-chloro-9-ethyl-9H-purin-6-yl)morpholin-3-yl)methanol

A mixture of 2,6-dichloro-9-ethyl-9H-purine (650 mg, 3 mmol), (S)-morpholin-3-ylmethanol (526 mg, 4.5 mmol), DIPEA (774 mg, 6 mmol) in acetonitrile (3 mL) was stirred at room temperature for 16 h. The reaction mixture was concentrated to give (S)-(4-(2-chloro-9-ethyl-9H-purin-6-yl)morpholin-3-yl)methanol (700 mg, 78%) as white solid. LCMS (ESI) m/z: 298.0 [M+H]⁺.

### Step 2: Synthesis of (S)-(4-(9-ethyl-2-(3-methoxy-4-phenyl-1H-pyrazol-1-yl)-9H-purin-6-yl)morpholin-3-yl)methanol

A mixture of (S)-(4-(2-chloro-9-ethyl-9H-purin-6-yl)morpholin-3-yl)methanol (200 mg, 0.67 mmol), 3-methoxy-4-phenyl-1H-pyrazole (174 mg, 1 mmol) and cesium carbonate (435 mg, 1.34 mmol) in N,N-dimethylformamide (5 mL) was stirred at 110° C. for 16 h. The resultant mixture was filtered through a pad of celite, the filtrate was concentrated and the crude product thus obtained was purified by column chromatography on silica gel (petroleum ether/ethyl acetate 5:1) to obtain (S)-(4-(9-ethyl-2-(3-methoxy-4-phenyl-1H-pyrazol-1-yl)-9H-purin-6-yl)morpholin-3-yl)methanol (61 mg, 21%) as white solid. ¹H NMR (500 MHz, DMSO-d₆) δ 8.88 (s, 1H), 8.20 (s, 1H), 7.79 (d, J=7.6 Hz, 2H), 7.40 (t, J=7.7 Hz, 2H), 7.25 (t, J=7.4 Hz, 1H), 5.73 (bs, 1H), 4.55 (d, J=301.5 Hz, 2H), 4.23 (q, J=7.3 Hz, 2H), 4.09 (s, 4H), 3.98 (d, J=8.4 Hz, 1H), 3.84 (t, J=9.6 Hz, 1H), 3.63 (dd, J=11.4, 3.2 Hz, 2H), 3.58-3.42 (m, 2H), 1.46 (t, J=7.3 Hz, 3H). LCMS (ESI) m/z: 436.2 [M+H]⁺.

### Synthesis of (R)-(4-(9-ethyl-2-(3-methoxy-4-phenyl-1H-pyrazol-1-yl)-9H-purin-6-yl)morpholin-3-yl)methanol (Compound 410)

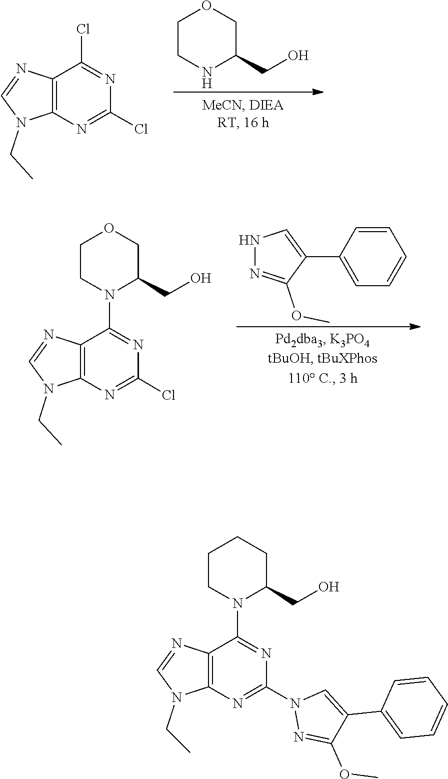

### Step 1: Synthesis of (R)-(4-(2-chloro-9-ethyl-9H-purin-6-yl)morpholin-3-yl)methanol

A mixture of 2,6-dichloro-9-ethyl-9H-purine (650 mg, 3 mmol), (R)-morpholin-3-ylmethanol (526 mg, 4.5 mmol) and N,N-diisopropylethylamine (774 mg, 6 mmol) in acetonitrile (3 mL) was stirred at room temperature for 16 h. The mixture was concentrated to give (R)-(4-(2-chloro-9-ethyl-9H-purin-6-yl)morpholin-3-yl)methanol (750 mg, 84%) as white solid. LCMS (ESI) m/z: 298.0 [M+H]⁺. It was taken to the next step without further purification.

### Step 2: Synthesis of (R)-(4-(9-ethyl-2-(3-methoxy-4-phenyl-1H-pyrazol-1-yl)-9H-purin-6-yl)morpholin-3-yl)methanol

A mixture of (R)-(4-(2-chloro-9-ethyl-9H-purin-6-yl)morpholin-3-yl)methanol (100 mg, 0.33 mmol), 3-methoxy-4-phenyl-1H-pyrazole (87 mg, 0.51 mmol), potassium phosphate tribasic (104 mg, 0.5 mmol), ditert-butyl-[2-[2,4,6-tri(propan-2-yl)phenyl]phenyl]phosphane (29 mg, 0.07 mmol) and tris(dibenzylideneacetone)dipalladium (31 mg, 0.033 mmol) in tert-butanol (3 mL) was stirred at 110° C. for 3 h. The resultant mixture was filtered through a pad of celite and the filtrated was concentrated. The crude product obtained was subjected to column chromatography on silica gel (petroleum ether/ethyl acetate 5:1) to afford (R)-(4-(9-ethyl-2-(3-methoxy-4-phenyl-1H-pyrazol-1-yl)-9H-purin-6-yl)morpholin-3-yl)methanol (100 mg, 70%) as white solid. 1H NMR (500 MHz, DMSO-d₆) δ 8.88 (s, 1H), 8.20 (s, 1H), 7.79 (d, J=7.6 Hz, 2H), 7.40 (t, J=7.7 Hz, 2H), 7.25 (t, J=7.4 Hz, 1H), 5.74 (s, 1H), 4.85 (s, 2H), 4.23 (q, J=7.3 Hz, 2H), 4.09 (s, 4H), 3.98 (d, J=8.7 Hz, 1H), 3.84 (t, J=9.6 Hz, 1H), 3.67-3.61 (m, 2H), 3.56 (dd, J=11.8, 9.2 Hz, 2H), 1.46 (t, J=7.3 Hz, 3H). LCMS (ESI) m/z: 436.0 [M+H]⁺.

### Synthesis of 9-ethyl-6-morpholino-N-(3-phenyl-1H-pyrazol-5-yl)-9H-purin-2-amine (Compound

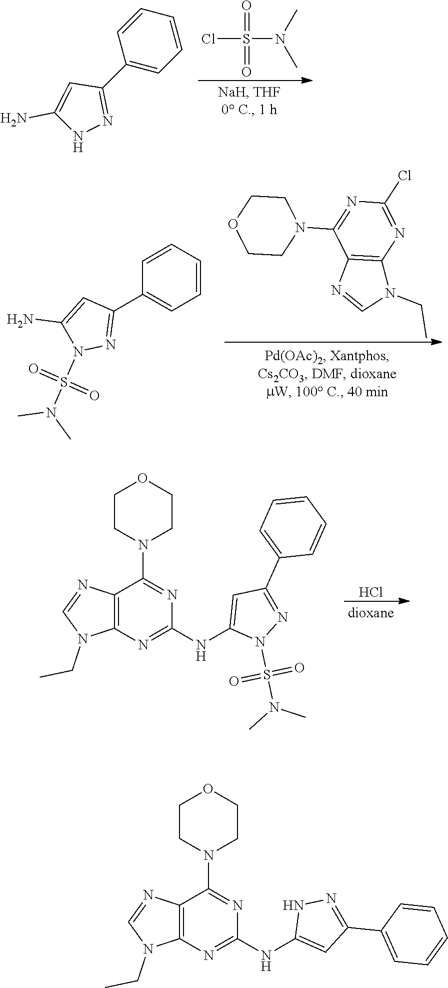

Step 1: Synthesis of 5-amino-N,N-dimethyl-3-phenyl-1H-pyrazole-1-sulfonamide. To a stirred solution of 3-phenyl-1H-pyrazol-5-amine (1.59 g, 10 mmol) in tetrahydrofuran (15 mL) at 0° C., was added sodium hydride (800 mg, 20 mmol) in portions and the resultant slurry was stirred at 0° C. for 1 h. Then dimethylsulfamoyl chloride (1.728 g, 12 mmol) was added and the resultant mixture was stirred at 20° C. for 1 h. The reaction was quenched with aqueous ammonium chloride solution (100 mL) and extracted with ethyl acetate (100 mL×3) The combined organic layers was dried over sodium sulfate, filtered and concentrated. The residue was subjected to flash column chromatography on silica gel (eluted with 20-33% ethyl acetate/petroleum ether) to obtain 5-amino-N,N-dimethyl-3-phenyl-1H-pyrazole-1-sulfonamide (1.19 g, 45%) as yellow solid. LCMS (ESI) m/z: 267.1 [M+H]⁺.

### Step 2: Synthesis of 5-((9-ethyl-6-morpholino-9H-purin-2-yl)amino)-N,N-dimethyl-3-phenyl-1H-pyrazole-1-sulfonamide

A solution of 5-amino-N,N-dimethyl-3-phenyl-1H-pyrazole-1-sulfonamide (150 mg, 0.564 mmol), 4-(2-chloro-9-ethyl-9H-purin-6-yl)morpholine (100 mg, 0.374 mmol), palladium (II) acetate (10 mg, 0.045 mmol), 4,5-bis(diphenylphosphino)-9,9-dimethylxanthene (43 mg, 0.074 mmol) and cesium carbonate (300 mg, 0.92 mmol) in 1,4-dioxane/N,N-dimethylformamide (1 mL/7 mL) was stirred at 100° C. for 1 h in microwave reactor under nitrogen atmosphere. The mixture was filtered, concentrated and purified by silica gel chromatography eluting with a linear gradient of 0% to 37% ethyl acetate in petroleum ether afford 5-((9-ethyl-6-morpholino-9H-purin-2-yl)amino)-N,N-dimethyl-3-phenyl-1H-pyrazole-1-sulfonamide (265 mg, 94.5%) as white solid. LCMS (ESI) m/z: 498.1 [M+H]⁺.

### Step 3: Synthesis of 9-ethyl-6-morpholino-N-(3-phenyl-1H-pyrazol-5-yl)-9H-purin-2-amine

A solution of 5-((9-ethyl-6-morpholino-9H-purin-2-yl)amino)-N,N-dimethyl-3-phenyl-1H-pyrazole-1-sulfonamide (215 mg, 0.433 mmol) and hydrochloric acid (0.5 mL, 20%) in 1,4-dioxane (5 mL) was stirred at room temperature for 16 h under nitrogen atmosphere. The mixture was filtered, concentrated and purified by prep-HPLC (BOSTON pHlex ODS 10 μm 21.2×250 mm 120 A). The mobile phase was acetonitrile/0.1% ammonium bicarbonate) to obtain 9-ethyl-6-morpholino-N-(3-phenyl-1H-pyrazol-5-yl)-9H-purin-2-amine (48.8 mg, 28.9%) as white solid. ¹H NMR (400 MHz, CDCl3) δ 12.16 (bs, 1H), 7.79 (d, J=7.4 Hz, 2H), 7.60 (s, 1H), 7.42 (t, J=7.5 Hz, 2H), 7.33 (dd, J=9.1, 5.5 Hz, 2H), 6.16 (bs, 1H), 4.49-4.10 (m, 6H), 3.83 (t, J=4.0 Hz, 4H), 1.53 (t, J=7.3 Hz, 3H). LCMS (ESI) m/z: 391.2 [M+H]⁺.

### Synthesis of 4-(2-(4-(3,4-difluorophenyl)-3-methoxy-1H-pyrazol-1-yl)-9-ethyl-9H-purin-6-yl)morpholine (Compound 412)

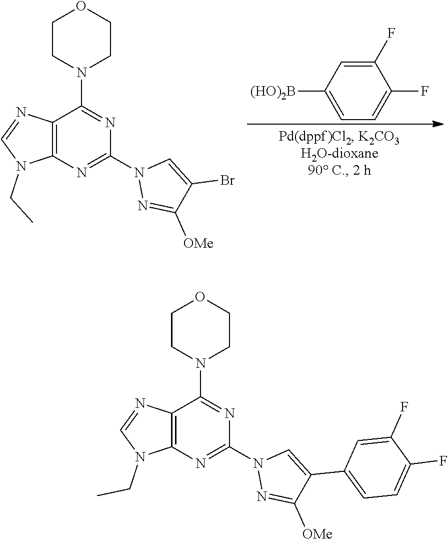 [SMILES: CCn1cnc2c(N3CCOCC3)nc(-n3cc(Br)c(OC)n3)nc21 ; OB(O)c1ccc(F)c(F)c1 ; CCn1cnc2c(N3CCOCC3)nc(-n3cc(-c4ccc(F)c(F)c4)c(OC)n3)nc21]

A mixture of 4-(2-(4-bromo-3-methoxy-1H-pyrazol-1-yl)-9-ethyl-9H-purin-6-yl)morpholine (150 mg, 0.368 mmol), (3,4-difluorophenyl)boronic acid (70 mg, 0.442 mmol), 1,1′-bis(diphenylphosphino)ferrocene-palladium(II)dichloride dichloromethane complex (30 mg, 0.037 mmol), and potassium carbonate (203 mg, 1.472 mmol) in 1,4-dioxane/water (5 mL/0.5 mL) was stirred at 90° C. for 2 h under nitrogen atmosphere. The resultant mixture was filtered, concentrated and purified by prep-HPLC (BOSTON pHlex ODS 10 μm 21.2×250 mm 120 A. The mobile phase was acetonitrile/0.1% ammonium bicarbonate) to obtain 4-(2-(4-(3,4-difluorophenyl)-3-methoxy-1H-pyrazol-1-yl)-9-ethyl-9H-purin-6-yl)morpholine (60.2 mg, 37.22%) as white solid. ¹H NMR (400 MHz, DMSO-d₆) δ 9.02 (s, 1H), 8.22 (s, 1H), 7.89 (ddd, J=12.5, 7.9, 2.0 Hz, 1H), 7.71-7.67 (m, 1H), 7.46 (dt, J=10.6, 8.8 Hz, 1H), 4.25 (bs, 4H), 4.22 (q, J=7.3 Hz, 2H), 4.08 (s, 3H), 3.79-3.75 (m, 4H), 1.45 (t, J=7.3 Hz, 3H). LCMS (ESI) m/z: 442.2 [M+H]⁺.

### Synthesis of 4-(9-ethyl-2-(3-methoxy-4-(3-methoxyphenyl)-1H-pyrazol-1-yl)-9H-purin-6-yl)morpholine (Compound 413)

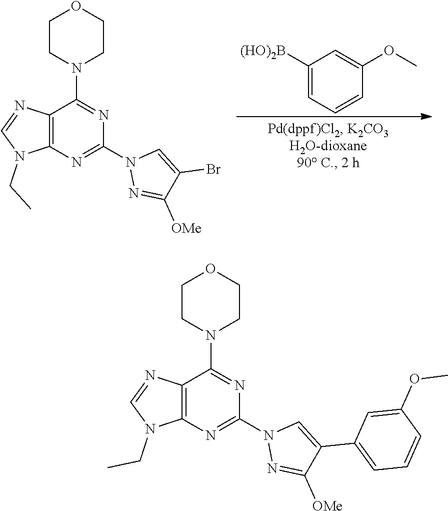 [SMILES: CCn1cnc2c(N3CCOCC3)nc(-n3cc(Br)c(OC)n3)nc21 ; COc1cccc(B(O)O)c1 ; CCn1cnc2c(N3CCOCC3)nc(-n3cc(-c4cccc(OC)c4)c(OC)n3)nc21]

A solution of 4-(2-(4-bromo-3-methoxy-1H-pyrazol-1-yl)-9-ethyl-9H-purin-6-yl)morpholine (60 mg, 0.147 mmol), (3-methoxyphenyl)boronic acid (27 mg, 0.176 mmol), 1,1′-bis(diphenylphosphino)ferrocene-palladium(II)dichloride dichloromethane complex (12 mg, 0.015 mmol), and potassium carbonate (82 mg, 0.588 mmol) in 1,4-dioxane/water (5 mL/0.5 ml) was stirred at 90° C. for 2 h under nitrogen atmosphere. The resultant mixture was filtered, concentrated and purified by prep-HPLC (BOSTON pHlex ODS 10 μm 21.2×250 mm 120 A. The mobile phase was acetonitrile/0.1% ammonium bicarbonate) to obtain 4-(9-ethyl-2-(3-methoxy-4-(3-methoxyphenyl)-1H-pyrazol-1-yl)-9H-purin-6-yl)morpholine (23.5 mg, 36.74%) as white solid. ¹H NMR (400 MHz, DMSO-d₆) δ 8.93 (s, 1H), 8.21 (s, 1H), 7.42-7.36 (m, 2H), 7.31 (t, J=7.9 Hz, 1H), 6.83 (dd, J=8.1, 2.0 Hz, 1H), 4.25 (bs, 4H), 4.21 (q, J=7.3 Hz, 2H), 4.07 (s, 3H), 3.80 (s, 3H), 3.79-3.75 (m, 4H), 1.45 (t, J=7.3 Hz, 3H). LCMS (ESI) m/z: 436.1 [M+H]⁺.

### Synthesis of 4-(9-ethyl-2-(3-methoxy-4-(pyridin-3-yl)-1H-pyrazol-1-yl)-9H-purin-6-yl)morpholine (Compound 414)

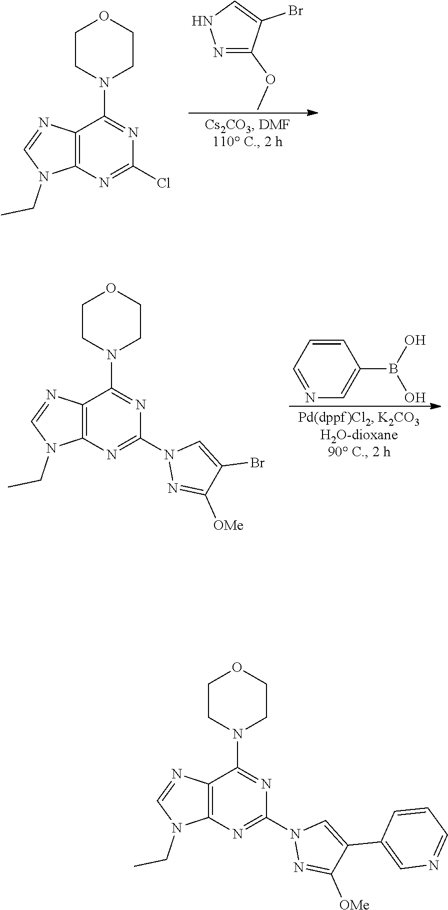

### Step 1: Synthesis of 4-(2-(4-bromo-3-methoxy-1H-pyrazol-1-yl)-9-ethyl-9H-purin-6-yl)morpholine

To a solution of 4-(2-chloro-9-ethyl-9H-purin-6-yl)morpholine (150 mg, 0.56 mmol) in DMF (8 mL) were added 4-bromo-3-methoxy-1H-pyrazole (119.0 mg, 0.67 mmol) and cesium carbonate (547.6 mg, 1.68 mmol) and the reaction mixture was stirred at 110° C. for 2 h under nitrogen protection. The mixture was extracted with ethyl acetate (20 mL*2), the organic layer was washed with water (10 mL*2), dried over sodium sulfate, and concentrated. The crude product thus obtained was purified by silica gel column chromatography (2% methanol in dichloromethane) to obtain 4-(2-(4-bromo-3-methoxy-1H-pyrazol-1-yl)-9-ethyl-9H-purin-6-yl)morpholine as white solid (100.0 mg, 43.9%). LCMS (ESI) m/z: 408.1/410 [M+H]⁺.

### Step 2: Synthesis of 4-(9-ethyl-2-(3-methoxy-4-(pyridin-3-yl)-1H-pyrazol-1-yl)-9H-purin-6-yl)morpholine

To a solution of 4-(2-(4-bromo-3-methoxy-1H-pyrazol-1-yl)-9-ethyl-9H-purin-6-yl)morpholine (70 mg, 0.17 mmol) in 1,4-dioxane (1.5 mL) and water (0.5 mL) were added pyridin-3-ylboronic acid (25.3 mg, 0.21 mmol), [1,1′-bis(diphenylphosphino)ferrocene]dichloropalladium(II) (12.5 mg, 0.01 mmol) and potassium carbonate (71.2 mg, 0.51 mmol) at 25° C. and the reaction mixture was heated and stirred at 90° C. for 2 h under nitrogen protection. The mixture was extracted with ethyl acetate (20 mL*2), the organic layer was washed with water (10 mL*2), dried over sodium sulfate, and concentrated. The crude product thus obtained was purified by prep-HPLC (BOSTON pHlex ODS 10 um 21.2×250 mm 120 A. The mobile phase was acetonitrile/0.1% ammonium bicarbonate) to obtain 4-(9-ethyl-2-(3-methoxy-4-(pyridin-3-yl)-1H-pyrazol-1-yl)-9H-purin-6-yl)morpholine as white solid (44.0 mg, 63.7%). ¹H NMR (400 MHz, DMSO-d₆) δ 9.13-9.01 (m, 2H), 8.49-8.41 (m, 1H), 8.22 (s, 1H), 8.18 (d, J=8.0 Hz, 1H), 7.43 (dd, J=7.9, 4.8 Hz, 1H), 4.35 (bs, 4H), 4.22 (q, J=7.3 Hz, 6H), 4.09 (s, 3H), 3.77 (t, J=4.0 Hz, 4H), 1.46 (t, J=7.3 Hz, 3H). LCMS (ESI) m/z: 407.1 [M+H]⁺.

The following compounds were synthesized according to the protocol described above:

| | | | Comp |
|---|---|---|---|
| Name | Structure | NMR, MS | # |
| 4-(9-ethyl-2-(4-(3- fluorophenyl)-3- methoxy-1H-pyrazol- 1-yl)-9H-purin-6- yl)morpholine | 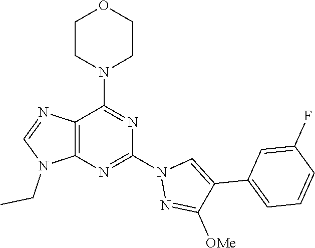 | ¹H NMR (400 MHz, DMSO-d₆) δ 9.04 (s, 1H), 8.22 (s, 1H), 7.68 (t, J = 8.6 Hz, 2H), 7.44 (dd, J = 14.7, 8.1 Hz, 1H), 7.07 (t, J = 8.4 Hz, 1H), 4.26 (bs, 4H), 4.23 (q, J = 7.2 Hz, 2H), 4.08 (s, 3H), 3.80-3.74 (m, 4H), 1.45 (t, J = 7.3 Hz, 3H). LCMS (ESI) m/z: 424.2 [M + H]⁺. | 415 |
| 4-(9-ethyl-2-(3- methoxy-4-(3- (trifluoromethyl)phenyl)- 1H-pyrazol-1-yl)-9H- purin-6-yl)morpholine | 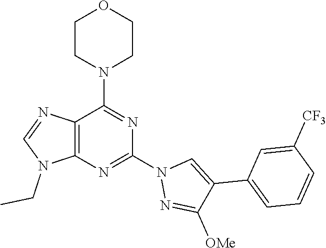 | ¹H NMR (400 MHz, DMSO-d₆) δ 9.11 (s, 1H), 8.23 (s, 1H), 8.15 (d, J = 7.3 Hz, 2H), 7.67-7.58 (m, 2H), 4.26 (bs, 4H), 4.23 (q, J = 7.2 Hz, 2H), 4.10 (s, 3H), 3.78 (d, J = 4.5 Hz, 4H), 1.46 (t, J = 7.3 Hz, 3H). LCMS (ESI) m/z: 474.1 [M + H]⁺. | 416 |
| 4-(9-ethyl-2-(3- methoxy-4-(5- methoxypyridin-3-yl)- 1H-pyrazol-1-yl)-9H- purin-6-yl)morpholine | 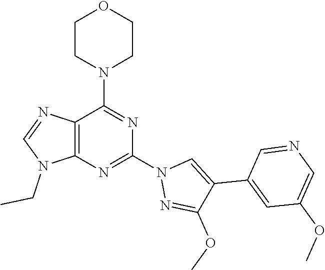 | ¹H NMR (400 MHz, DMSO-d₆) δ 9.10 (s, 1H), 8.68 (d, J = 1.7 Hz, 1H), 8.22 (s, 1H), 8.18 (d, J = 2.8 Hz, 1H), 7.74 (dd, J = 2.7, 1.9 Hz, 1H), 4.38 (bs, 4H), 4.23 (q, J = 7.2 Hz, 2H), 4.09 (s, 3H), 3.89 (s, 3H), 3.79-3.75 (m, 4H), 1.46 (t, J = 7.2 Hz, 3H). LCMS (ESI) m/z: 437.1 [M + H]⁺. | 417 |

### Synthesis of 9-ethyl-2-(3-methoxy-4-phenyl-1H-pyrazol-1-yl)-6-(pyridin-4-yl)-9H-purine (Compound 418)

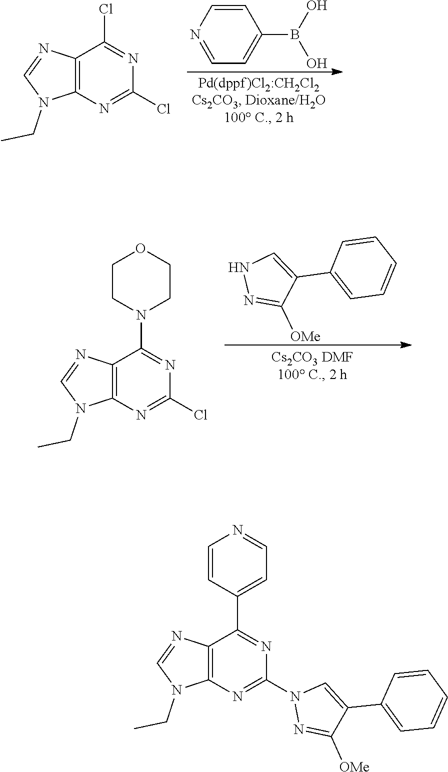 [SMILES: CCn1cnc2c(Cl)nc(Cl)nc21 ; OB(O)c1ccncc1 ; CCn1cnc2c(N3CCOCC3)nc(Cl)nc21 ; COc1n[nH]cc1-c1ccccc1 ; CCn1cnc2c(-c3ccncc3)nc(-n3cc(-c4ccccc4)c(OC)n3)nc21]

### Step 1: Synthesis of 2-chloro-9-ethyl-6-(pyridin-4-yl)-9H-purine

A mixture of 2,6-dichloro-9-ethyl-9H-purine (1.08 g, 5.0 mmol), pyridin-4-ylboronic acid (615 mg, 5.0 mmol), 1,1′-bis(diphenyl phosphino)ferrocene-palladium(II)dichloride dichloromethane complex (204 mg, 0.25 mmol) and cesium carbonate (3.25 g, 10.0 mmol), 1,4-dioxane (20 mL) and water (4.0 mL) was stirred at 100° C. under nitrogen atmosphere for 2 h. The resultant mixture was poured into water and extracted with ethyl acetate (200 mL*2). The combined organic phase was concentrated and purified by silica gel column chromatography (100% ethyl acetate) to obtain 2-chloro-9-ethyl-6-(pyridin-4-yl)-9H-purine (650 mg, 50%) as purple solid. ¹H NMR (400 MHz, C0013) b 8.84 (dd, J=4.6, 1.6 Hz, 2H), 8.63 (dd, J=4.5, 1.6 Hz, 2H), 8.19 (s, 1H), 4.38 (q, J=7.4 Hz, 2H), 1.61 (t, J=7.4 Hz, 3H); LCMS (ESI) m/z: 260.1/262.0 [M+H]⁺.

### Step 2: Synthesis of 9-ethyl-2-(3-methoxy-4-phenyl-1H-pyrazol-1-yl)-6-(pyridin-4-yl)-9H-purine

A mixture of 2-chloro-9-ethyl-6-(pyridin-4-yl)-9H-purine (250 mg, 1.0 mmol), 3-methoxy-4-phenyl-1H-pyrazole (174 mg, 1.0 mmol) and cesium carbonate (650 mg, 2.0 mmol) in DMF (5 mL) was stirred at 100° C. for 2 h. The mixture was filtered, and the filtrate was poured into water. The resultant precipitate was collected by filtration and dried under vacuum to afford 280 mg of a yellow solid, which was further purified by silica gel column chromatography [(10% methanol in dichloromethane) and washed with methanol (10 mL)] to obtain 4-(2-(3-methoxy-4-phenyl-1H-pyrazol-1-yl)pyrido[3,2-d]pyrimidin-4-yl)morpholine (175 mg, 22%) as light yellow solid. ¹H NMR (400 MHz, DMSO-d₆) δ 9.23 (s, 1H), 8.87 (s, 4H), 8.76 (s, 1H), 7.89 (d, J=7.5 Hz, 2H), 7.44 (t, J=7.7 Hz, 2H), 7.29 (t, J=7.4 Hz, 1H), 4.37 (q, J=7.3 Hz, 2H), 4.13 (s, 3H), 1.55 (t, J=7.3 Hz, 3H); LCMS (ESI) m/z: 398.3 [M+H]⁺.

### Synthesis of 4-(9-ethyl-2-(3-methoxy-4-(pyridin-2-yl)-1H-pyrazol-1-yl)-9H-purin-6-yl)morpholine (Compound 419)

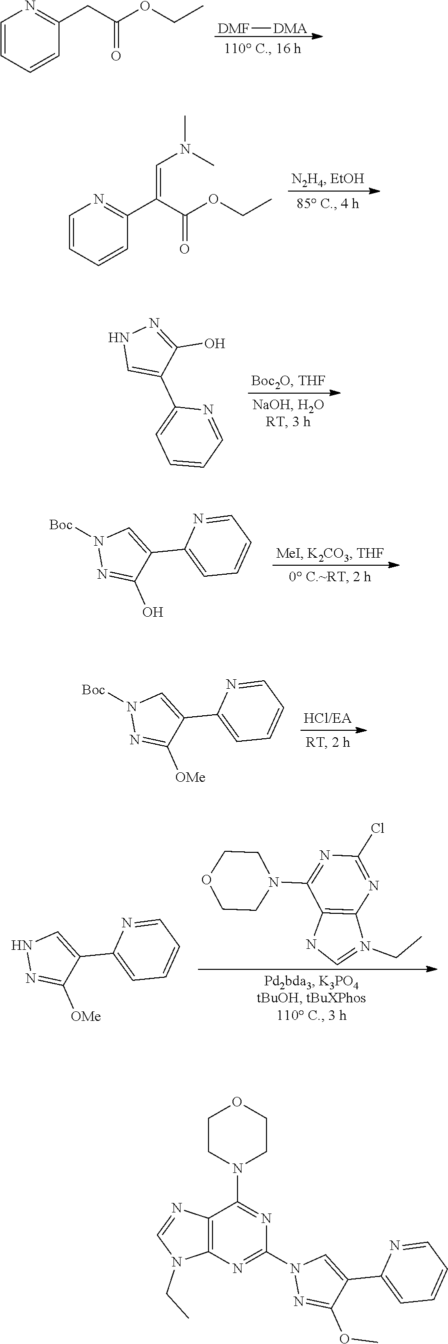

### Step 1: Synthesis of (Z)-3-(dimethylamino)-2-(pyridin-2-yl)acrylate

A solution of ethyl 2-(pyridin-2-yl)acetate (5 g, 30 mmol) in 1,1-dimethoxy-N,N-dimethylmethanamine (40 mL) was stirred at 110° C. for 17 h. The resultant mixture was concentrated to obtain ethyl (Z)-3-(dimethylamino)-2-(pyridin-2-yl)acrylate (8 g, quant.) as a yellow oil. LCMS (ESI) m/z: 222.1 [M+H]⁺. This crude product was taken to the next step without further purification.

### Step 2: Synthesis of 4-(pyridin-2-yl)-1H-pyrazol-3-ol

A mixture of ethyl (Z)-3-(dimethylamino)-2-(pyridin-2-yl)acrylate (8 g, 36 mmol) and hydrazine hydrate (6.75 mL, 108 mmol) in ethanol (60 mL) was stirred at reflux for 2 h. The reaction mixture was then concentrated to obtain 4-(pyridin-2-yl)-1H-pyrazol-3-ol (6 g, quant.) as yellow oil, which was used directly at next step. LCMS (ESI) m/z: 162.1 [M+H]⁺.

### Step 3: Synthesis of tert-butyl 3-hydroxy-4-(pyridin-2-yl)-1H-pyrazole-1-carboxylate

A mixture of 4-(pyridin-2-yl)-1H-pyrazol-3-ol (6 g, 37 mmol), di-tert-butyl dicarbonate (16 g, 74 mmol) and sodium hydroxide (2.5 mol/L in water, 30 mL) in tetrahydrofuran (30 mL) was stirred at room temperature for 16 h. The resultant mixture was partitioned between ethyl acetate (200 mL) and water (200 mL). The organic layer was washed with brine and evaporated to dryness. The crude product was purified by column chromatography on silica gel (petroleum ether/ethyl acetate 20:1→10:1→5:1) to give the tert-butyl 3-hydroxy-4-(pyridin-2-yl)-1H-pyrazole-1-carboxylate (6 g, 62%) as white solid. LCMS (ESI) m/z: 262.2 [M+H]⁺.

### Step 4: Synthesis of tert-butyl 3-methoxy-4-(pyridin-2-yl)-1H-pyrazole-1-carboxylate

A mixture of tert-butyl 3-hydroxy-4-(pyridin-2-yl)-1H-pyrazole-1-carboxylate (1 g, 3.8 mmol), potassium carbonate (317 mg, 2.3 mmol) and iodomethane (324 mg, 2.3 mmol) in tetrahydrofuran (10 mL) was stirred at room temperature for 2 h. The resultant mixture was partitioned between ethyl acetate (50 mL) and water (50 mL). The organic layer was washed with brine, dried and evaporated to dryness. The crude product was purified by column chromatography on silica gel (petroleum ether/ethyl acetate 20:1→10:1→5:1) to obtain the tert-butyl 3-methoxy-4-(pyridin-2-yl)-1H-pyrazole-1-carboxylate (250 mg, 79%) as white solid. LCMS (ESI) m/z: 276.1 [M+H]⁺.

### Step 5: Synthesis of 2-(3-methoxy-1H-pyrazol-4-yl)pyridine

A mixture of tert-butyl 3-methoxy-4-(pyridin-2-yl)-1H-pyrazole-1-carboxylate (250 mg, 0.9 mmol) in hydrogen chloride methanol solution (4N, 3 mL) was stirred at room temperature for 2 h. The mixture was then concentrated to give 2-(3-methoxy-1H-pyrazol-4-yl)pyridine (100 mg, 64%) as a colorless oil. LCMS (ESI) m/z: 176.1 [M+H]⁺.

### Step 6: Synthesis of 4-(9-ethyl-2-(3-methoxy-4-(pyridin-2-yl)-1H-pyrazol-1-yl)-9H-purin-6-yl)morpholine

A mixture of 4-(2-chloro-9-ethyl-9H-purin-6-yl)morpholine (93 mg, 0.35 mmol), 2-(3-methoxy-1H-pyrazol-4-yl)pyridine (93 mg, 0.53 mmol) potassium phosphate (111 mg, 0.52 mmol) ditert-butyl-[2-[2,4,6-tri(propan-2-yl)phenyl]phenyl]phosphane (29 mg, 0.07 mmol) and tris(dibenzylideneacetone)dipalladium (32 mg, 0.035 mmol) in tert-butanol (3 mL) was stirred at 110° C. for 3 h. The resultant mixture was filtered through a pad of celite, and the filtrate was subjected to column chromatography on silica gel (petroleum ether/ethyl acetate 5:1) to obtain 4-(9-ethyl-2-(3-methoxy-4-(pyridin-2-yl)-1H-pyrazol-1-yl)-9H-purin-6-yl)morpholine (29.1 mg, 21%) as a white solid. ¹H NMR (500 MHz, DMSO-d₆) δ 8.96 (s, 1H), 8.58 (d, J=4.8 Hz, 1H), 8.23 (s, 1H), 7.83 (dd, J=5.9, 1.4 Hz, 2H), 7.29-7.21 (m, 1H), 4.26 (bs, 4H), 4.22 (q, J=7.3 Hz, 2H), 4.11 (s, 3H), 3.80-3.75 (m, 4H), 1.45 (t, J=7.3 Hz, 3H). LCMS (ESI) m/z: 407.1 [M+H]⁺.

### Synthesis of (R)-4-(9-ethyl-2-(3-methoxy-4-phenyl-1H-pyrazol-1-yl)-9H-purin-6-yl)-3-methylmorpholine (Compound 420)

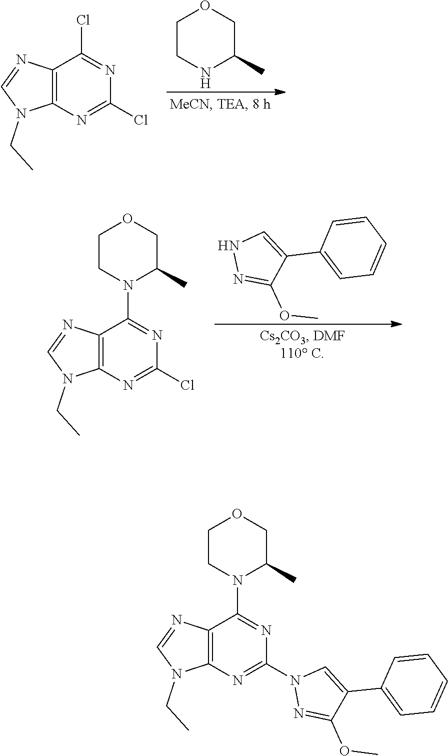

### Step 1: Synthesis of (R)-4-(2-chloro-9-ethyl-9H-purin-6-yl)-3-methylmorpholine

To a solution of 2,6-dichloro-9-ethyl-9H-purine (220 mg, 1.02 mmol) in acetonitrile (10 mL) were added (R)-3-methylmorpholine (128 mg, 1.273 mmol) and triethylamine (2 equivalent). The mixture was stirred at 30° C. for 8 h, then the reaction was quenched with water (10 mL) and extracted with ethyl acetate (15*3 mL). The organic layers were combined, washed with brine (50 mL), dried over sodium sulfate, filtered and concentrated. The obtained residue was subjected to flash chromatography on silica gel (petroleum ether:ethyl acetate=75:25) to obtain (R)-4-(2-chloro-9-ethyl-9H-purin-6-yl)-3-methylmorpholine as yellow solid. (170 mg, 59.2%). LCMS (ESI) m/z: 282.1 [M+H]⁺.

### Step 2: Synthesis of (R)-4-(9-ethyl-2-(3-methoxy-4-phenyl-1H-pyrazol-1-yl)-9H-purin-6-yl)-3-methylmorpholine

To a solution of (R)-4-(2-chloro-9-ethyl-9H-purin-6-yl)-3-methylmorpholine (170 mg, 0.605 mmol) in N,N-dimethylformamide (5 mL) were added 3-methoxy-4-phenyl-1H-pyrazole (175 mg, 1.01 mmol) and cesium carbonate (32 mg, 1.0 mmol). The resultant mixture was stirred at 110° C. for 8 h, then the reaction was quenched with water (15 mL) and extracted with ethyl acetate (20*3 mL). The organic layers were combined, washed with brine (30 mL), dried over sodium sulfate, filtered and concentrated. The resultant residue was subjected to prep-HPLC (0.05% ammonium bicarbonate:acetonitrile=5%-95%) to obtain (R)-4-(9-ethyl-2-(3-methoxy-4-phenyl-1H-pyrazol-1-yl)-9H-purin-6-yl)-3-methylmorpholine (26.2 mg, 10.3%) as white solid. ¹H NMR (400 MHz, DMSO-d₆) δ 8.90 (s, 1H), 8.21 (s, 1H), 7.83-7.76 (m, 2H), 7.40 (t, J=7.8 Hz, 2H), 7.25 (d, J=7.4 Hz, 1H), 5.63 (bs, 2H), 4.23 (q, J=7.3 Hz, 2H), 4.07 (s, 3H), 4.01 (d, J=8.2 Hz, 1H), 3.79 (d, J=11.4 Hz, 1H), 3.72 (dd, J=11.5, 2.9 Hz, 1H), 3.55 (dd, J=11.6, 2.5 Hz, 1H), 3.38 (d, J=37.0 Hz, 1H), 1.46 (t, J=7.3 Hz, 3H), 1.35 (d, J=6.8 Hz, 3H). LCMS (ESI) m/z: 420.3[M+H]⁺.

### Synthesis of 2-(2-(3-methoxy-4-phenyl-1H-pyrazol-1-yl)-6-morpholino-9H-purin-9-yl)ethan-1-ol (Compound 421)

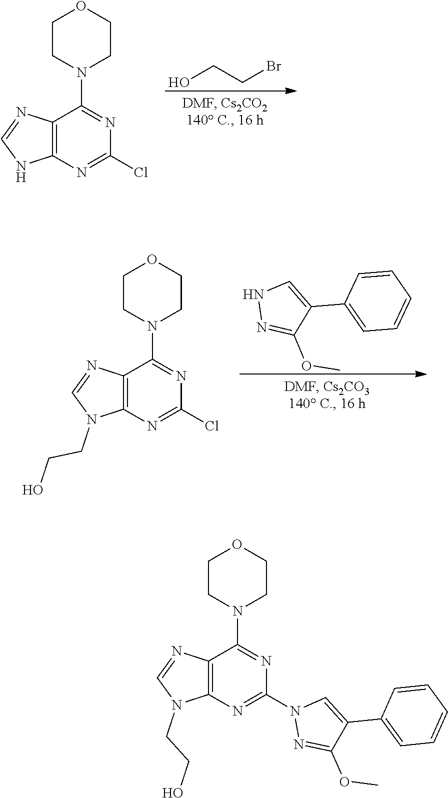

### Step 1: Synthesis of 2-(2-chloro-6-morpholino-9H-purin-9-yl)ethan-1-ol

To a mixture of 4-(2-chloro-9H-purin-6-yl)morpholine (150 mg, 0.63 mmol) and cesium carbonate (614 mg, 1.89 mmol) in DMF (10 mL) was added 2-bromoethan-1-ol (94 mg, 0.76 mmol) dropwise, and the resulting mixture was stirred at 140° C. for 16 h. The reaction mixture was concentrated, the residue was diluted with water (20 mL) and extracted with dichloromethane (20 mL×2). The combined organic phase was washed with brine (20 mL), dried over sodium sulfate, filtered and concentrated. The residue was subjected to flash chromatography (eluted with petroleum ether in ethyl acetate from 50% to 80%) to obtain 2-(2-chloro-6-morpholino-9H-purin-9-yl)ethan-1-ol (120 mg, 67%) as pale yellow oil. LCMS (ESI) m/z: 284.0 [M+H]⁺.

### Step 2: Synthesis of 2-(2-(3-methoxy-4-phenyl-1H-pyrazol-1-yl)-6-morpholino-9H-purin-9-yl)ethan-1-ol

To a mixture of 2-(2-chloro-6-morpholino-9H-purin-9-yl)ethan-1-ol (120 mg, 0.42 mmol), cesium carbonate (410 mg, 1.26 mmol) in N,N-dimethylformamide (10 mL) was added 3-methoxy-4-phenyl-1H-pyrazole (77 mg, 0.44 mmol), and the resulting mixture was stirred at 140° C. for 16 h. The reaction mixture was concentrated and subjected to prep-HPLC [(Welch Xtimate C18 21.2×250 mm, 10 um, with mobile phase acetonitrile/water (10 mM NH₄HCO₃ and NH₃·H₂O)] to afford 2-(2-(3-methoxy-4-phenyl-1H-pyrazol-1-yl)-6-morpholino-9H-purin-9-yl)ethan-1-ol (10.6 mg, 6%) as white solid. ¹H NMR (500 MHz, DMSO-d₆) δ 8.93 (s, 1H), 8.13 (s, 1H), 7.81 (d, J=7.3 Hz, 2H), 7.40 (t, J=7.8 Hz, 2H), 7.25 (d, J=7.4 Hz, 1H), 4.56-4.13 (m, 6H), 4.07 (s, 3H), 3.80 (t, J=5.4 Hz, 2H), 3.79-3.75 (m, 4H). LCMS (ESI) m/z: 422.1 [M+H]⁺.

### Synthesis of 4-(9-ethyl-2-(4-phenyl-3-(trifluoromethoxy)-1H-pyrazol-1-yl)-9H-purin-6-yl)morpholine (Compound 422)

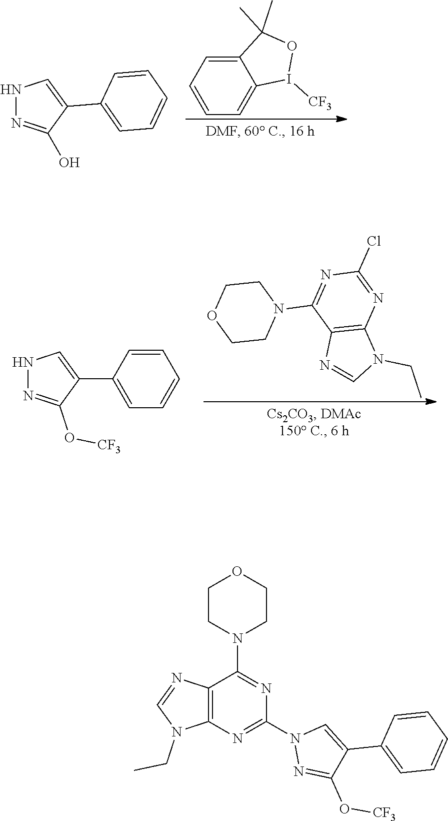

### Step 1: Synthesis of 4-phenyl-3-(trifluoromethoxy)-1H-pyrazole

A mixture of 4-phenyl-1H-pyrazol-3-ol (640 mg, 4 mmol), 1,3-dihydro-3,3-dimethyl-1-(trifluoromethyl)-1,2-benziodoxole (1.254 g, 3.8 mmol) in N,N-dimethylformamide (20 mL) was stirred at 60° C. for 16 h. The entire mixture was subjected to prep-HPLC (BOSTON pHlex ODS 10 μm 21.2×250 mm 120 A. The mobile phase was acetonitrile/0.1% ammonium bicarbonate) to afford 4-phenyl-3-(trifluoromethoxy)-1H-pyrazole (37 mg, 4%) as yellow solid. LCMS (ESI) m/z: 228.8 [M+H]⁺.

### Step 2: Synthesis of 4-(9-ethyl-2-(4-phenyl-3-(trifluoromethoxy)-1H-pyrazol-1-yl)-9H-purin-6-yl)morpholine

A mixture of 4-phenyl-3-(trifluoromethoxy)-1H-pyrazole (23 mg, 0.1 mmol), 4-(2-chloro-9-ethyl-9H-purin-6-yl)morpholine (32 mg, 0.12 mmol) and cesium carbonate (130 mg, 0.4 mmol) in DMAc (3 mL) was stirred at 150° C. for 6 h. The resultant reaction mixture was subjected to prep-HPLC (BOSTON pHlex ODS 10 um 21.2×250 mm 120 A. The mobile phase was acetonitrile/0.1% ammonium bicarbonate) to obtain 4-(9-ethyl-2-(4-phenyl-3-(trifluoromethoxy)-1H-pyrazol-1-yl)-9H-purin-6-yl)morpholine (20.5 mg, 45%) as white solid. ¹H NMR (400 MHz, DMSO-d₆) δ 9.10 (s, 1H), 8.28 (s, 1H), 7.72 (d, J=7.2 Hz, 2H), 7.48 (t, J=7.7 Hz, 2H), 7.37 (t, J=7.4 Hz, 1H), 4.60-4.30 (m, 4H), 4.24 (q, J=7.3 Hz, 2H), 3.77 (t, J=4.8 Hz, 4H), 1.45 (t, J=7.3 Hz, 3H). LCMS (ESI) m/z: 460.1 [M+H]⁺.

### Synthesis of 9-ethyl-2-(3-(1-methyl-1H-pyrazol-3-yl)phenyl)-6-(pyridin-3-yl)-9H-purine (Compound 423)

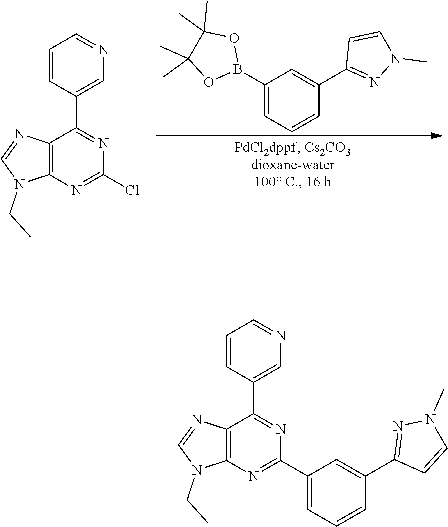

To a solution of 2-chloro-9-ethyl-6-(pyridin-3-yl)-9H-purine (100 mg, 0.38 mmol) in 1,4-dioxane/water (10 mL/1 mL) were added 1-methyl-3-(3-(4,4,5,5-tetramethyl-1,3,2-dioxaborolan-2-yl)phenyl)-1H-pyrazole (110 mg, 0.38 mmol), cesium carbonate (370 mg, 1.14 mmol) and 1,1′-bis(diphenylphosphino)ferrocene]dichloropalladium(II) (31 mg, 0.038 mmol) at 25° C. and the resultant reaction mixture was stirred at 100° C. for 16 h under argon. It was concentrated and the residue was subjected to Prep-HPLC [(Welch Xtimate C18 21.2×250 mm, 10 um, with mobile phase acetonitrile/water (10 mM NH₄HCO₃ and NH₃·H₂O)] to afford 9-ethyl-2-(3-(1-methyl-1H-pyrazol-3-yl)phenyl)-6-(pyridin-3-yl)-9H-purine (45.6 mg, 32%) as white solid. ¹H NMR (400 MHz, DMSO-d₆) δ 10.07 (s, 1H), 9.22 (d, J=8.1 Hz, 1H), 8.98 (s, 1H), 8.80 (d, J=7.8 Hz, 2H), 8.54 (d, J=7.7 Hz, 1H), 7.94 (d, J=7.7 Hz, 1H), 7.81 (d, J=2.2 Hz, 1H), 7.71 (dd, J=7.6, 5.0 Hz, 1H), 7.60 (t, J=7.7 Hz, 1H), 6.82 (d, J=2.2 Hz, 1H), 4.46 (q, J=7.3 Hz, 2H), 3.95 (s, 3H), 1.58 (t, J=7.3 Hz, 3H). LCMS (ESI) m/z: 382.2 [M+H]⁺.

### Synthesis of 9-ethyl-2-(3-methoxy-4-phenyl-1H-pyrazol-1-yl)-6-(pyridin-3-yl)-9H-purine (Compound 424)

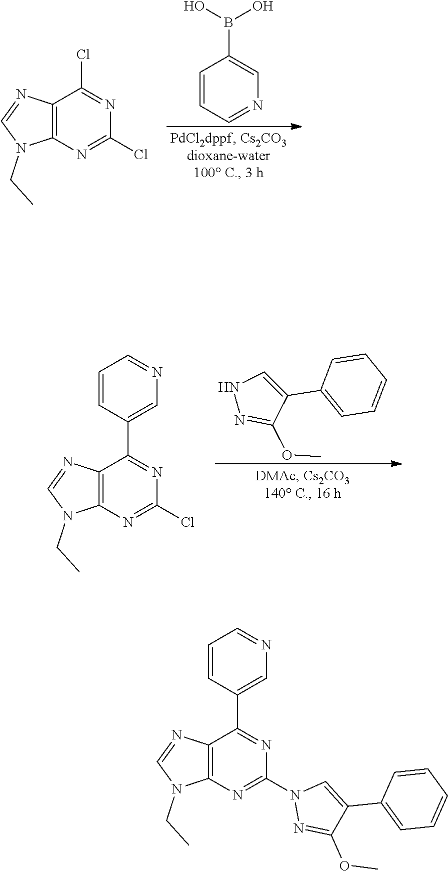 [SMILES: CCn1cnc2c(Cl)nc(Cl)nc21 ; OB(O)c1cccnc1 ; CCn1cnc2c(-c3cccnc3)nc(Cl)nc21 ; COc1n[nH]cc1-c1ccccc1 ; CCn1cnc2c(-c3cccnc3)nc(-n3cc(-c4ccccc4)c(OC)n3)nc21]

### Step 1: Synthesis of 2-chloro-9-ethyl-6-(pyridin-3-yl)-9H-purine

A mixture of 2,6-dichloro-9-ethyl-9H-purine (0.4 g, 1.85 mmol), pyridin-3-ylboronic acid (0.228 g, 1.85 mmol), 1,1′-bis(diphenylphosphino)ferrocene-palladium(II)dichloride dichloromethane complex (0.15 g, 0.185 mmol), and cesium carbonate (1.8 g, 5.55 mmol) in 1,4-dioxane/water (20 mL/3 mL) was stirred at 100° C. for 3 h under argon atmosphere. The reaction mixture was concentrated followed by the addition of water (20 mL), and extracted with dichloromethane (20 mL×2). The combined organic phase was washed with brine (20 mL), dried over sodium sulfate, filtered and concentrated. The residue was subjected to flash chromatography (eluted with petroleum ether in ethyl acetate from 50% to 80%) to afford 2-chloro-9-ethyl-6-(pyridin-3-yl)-9H-purine (0.2 g, 41.7%) as pale yellow solid. LCMS (ESI) m/z: 259.8 [M+H]⁺.

### Step 2: Synthesis of 9-ethyl-2-(3-methoxy-4-phenyl-1H-pyrazol-1-yl)-6-(pyridin-3-yl)-9H-purine

A mixture of 2-chloro-9-ethyl-6-(pyridin-3-yl)-9H-purine (100 mg, 0.38 mmol), cesium carbonate (378 mg, 1.16 mmol) and 3-methoxy-4-phenyl-1H-pyrazole (70 mg, 0.4 mmol) in DMF (10 mL) was stirred at 140° C. for 16 h under argon atmosphere. The mixture was concentrated and the residue was subjected to prep-HPLC [(Welch Xtimate C18 21.2×250 mm, 10 um, with mobile phase acetonitrile/water (10 mM NH₄HCO₃ and NH₃·H₂O)] to obtain 9-ethyl-2-(3-methoxy-4-phenyl-1H-pyrazol-1-yl)-6-(pyridin-3-yl)-9H-purine as white solid (64.7 mg, 42%). ¹H NMR (500 MHz, DMSO-d₆) δ 10.13 (s, 1H), 9.30 (s, 1H), 9.28 (s, 1H), 8.81 (d, J=4.7 Hz, 1H), 8.75 (s, 1H), 7.90 (d, J=7.9 Hz, 2H), 7.70 (s, 1H), 7.43 (t, J=7.7 Hz, 2H), 7.28 (t, J=7.3 Hz, 1H), 4.38 (q, J=7.3 Hz, 2H), 4.14 (s, 3H), 1.55 (t, J=7.3 Hz, 3H). LCMS (ESI) m/z: 398.1 [M+H]⁺.

### Synthesis of 2-(9-ethyl-2-(3-methoxy-4-phenyl-1H-pyrazol-1-yl)-6-morpholino-9H-purin-8-yl)-N,N-dimethylacetamide (Compound 425)

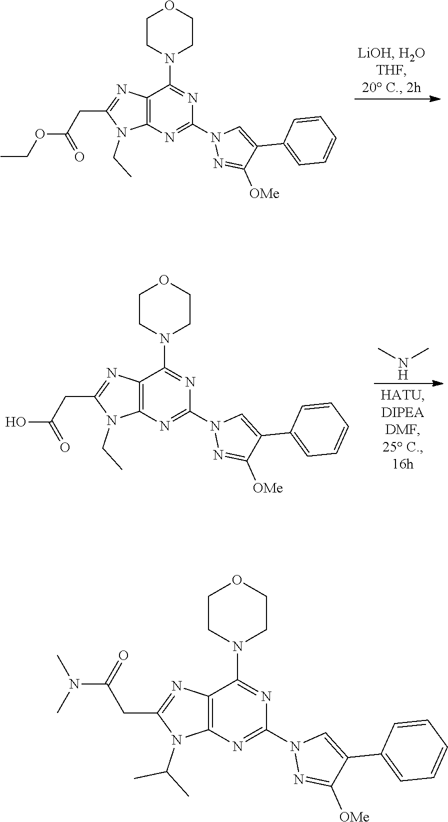

### Step 1: Synthesis of 2-(9-ethyl-2-(3-methoxy-4-phenyl-1H-pyrazol-1-yl)-6-morpholino-9H-purin-8-yl)acetic acid

To a solution of ethyl 2-(9-ethyl-2-(3-methoxy-4-phenyl-1H-pyrazol-1-yl)-6-morpholino-9H-purin-8-yl)acetate (0.21 g, 0.43 mmol) in tetrahydrofuran/water (10 mL/2 mL) was added Lithium hydroxide monohydrate (36 mg, 0.86 mmol) in portions at room temperature. After the addition, the reaction mixture was stirred for 2 h and concentrated to afford crude 2-(9-ethyl-2-(3-methoxy-4-phenyl-1H-pyrazol-1-yl)-6-morpholino-9H-purin-8-yl)acetic acid (0.2 g, quant.) as yellow solid. LCMS (ESI) m/z: 464.1 [M+H]⁺.

### Step 2: Synthesis of 2-(9-ethyl-2-(3-methoxy-4-phenyl-1H-pyrazol-1-yl)-6-morpholino-9H-purin-8-yl)-N,N-dimethylacetamide

To a solution of 2-(9-ethyl-2-(3-methoxy-4-phenyl-1H-pyrazol-1-yl)-6-morpholino-9H-purin-8-yl)acetic acid (80 mg, 0.17 mmol) in N,N-dimethylformamide (5 mL) were added dimethylamine hydrochloride (17 mg, 0.21 mmol) and HATU (98 mg, 0.26 mmol), followed by N,N-diisopropylethylamine (67 mg, 0.52 mmol). After the addition, the reaction mixture was stirred at room temperature for 16 h, then filtered and filtrate was subjected to prep-HPLC (base) to afford 2-(9-ethyl-2-(3-methoxy-4-phenyl-1H-pyrazol-1-yl)-6-morpholino-9H-purin-8-yl)-N,N-dimethylacetamide (40 mg, 48%) as white solid. ¹H NMR (400 MHz, DMSO-d₆) δ 8.91 (s, 1H), 7.85-7.75 (m, 2H), 7.40 (t, J=7.8 Hz, 2H), 7.24 (d, J=7.4 Hz, 1H), 4.45-4.18 (m, 4H), 4.16-4.08 (m, 4H), 4.07 (s, 3H), 3.79-3.72 (m, 4H), 3.11 (s, 3H), 2.87 (s, 3H), 1.36 (t, J=7.2 Hz, 3H). LCMS (ESI) m/z: 491.2 [M+H]⁺.

### Synthesis of 2-(9-ethyl-2-(4-methoxy-3-(1-methyl-1H-pyrazol-3-yl)phenyl)-6-morpholino-9H-purin-8-yl)ethan-1-ol (Compound 426)

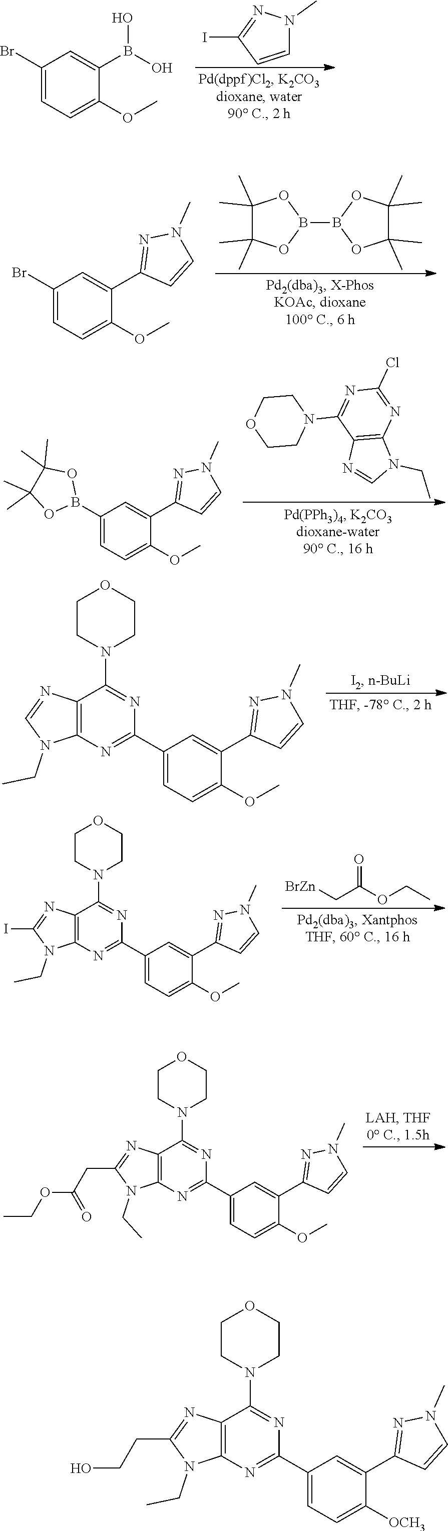

### Step 1: Synthesis of 3-(5-bromo-2-methoxyphenyl)-1-methyl-1H-pyrazole

To a solution of (5-bromo-2-methoxyphenyl)boronic acid (1 g, 4.33 mmol) in 1,4-dioxane (15 mL) and water (5 mL) were added 3-iodo-1-methyl-1H-pyrazole (1.17 mg, 5.63 mmol), potassium carbonate (1.2 g, 8.66 mmol) and 1,1′-bis(diphenylphosphino) ferrocene palladium(II)dichloride (0.314 g, 0.43 mmol). The resultant reaction mixture was stirred at 100° C. under nitrogen atmosphere for 2 h and diluted with ethyl acetate (50 mL). The organic layer was washed with water (50 mL*2) and brine (50 mL*1), dried over sodium sulfate, filtered and concentrated. The residue was subjected to flash chromatography (petroleum ether:ethyl acetate=3:1) to give 3-(5-bromo-2-methoxyphenyl)-1-methyl-1H-pyrazole (700 mg, 60%) as yellow solid. LCMS (ESI) m/z: 267.0 [M+H]⁺.

### Step 2: Synthesis of 3-(2-methoxy-5-(4,4,5,5-tetramethyl-1,3,2-dioxaborolan-2-yl)phenyl)-1-methyl-1H-pyrazole

A mixture of 3-(5-bromo-2-methoxyphenyl)-1-methyl-1H-pyrazole (0.7 g, 2.62 mmol), 4,4,4′,4′,5,5,5′,5′-octamethyl-2,2′-bi(1,3,2-dioxaborolane) (0.9 g, 3.54 mmol), potassium acetate (0.51 g, 5.24 mmol), tris(dibenzylideneacetone)dipalladium(0) (0.14 g, 0.26 mmol), and 2-(dicyclohexylphosphino)-2′,4′,6′-triisopropylbiphenyl (0.19 g, 0.39 mmol) in 1,4-dioxane (15 mL) was stirred at 85° C. for 6 h. The reaction mixture was filtered, concentrated and the residue was subjected to column chromatography (petroleum ether:ethyl acetate=7:1) to give 3-(2-methoxy-5-(4,4,5,5-tetramethyl-1,3,2-dioxaborolan-2-yl)phenyl)-1-methyl-1H-pyrazole as a yellow solid (0.45 g, 55%). LCMS (ESI) m/z: 315.1 [M+H]⁺.

### Step 3: Synthesis of 4-(9-ethyl-2-(4-methoxy-3-(1-methyl-1H-pyrazol-3-yl)phenyl)-9H-purin-6-yl)morpholine

To a solution of 4-(2-chloro-9-ethyl-9H-purin-6-yl)morpholine (0.38 g, 1.42 mmol) and 3-(2-methoxy-5-(4,4,5,5-tetramethyl-1,3,2-dioxaborolan-2-yl)phenyl)-1-methyl-1H-pyrazole (0.45 g, 1.42 mmol) in 1,4-dioxane/water (20 mL/6 mL) were added potassium carbonate (0.49 g, 3.55 mmol) and tetrakis(triphenylphosphine)palladium (0.16 g, 0.14 mmol). Then the reaction mixture was stirred at 90° C. for 2 h, then poured into ice-water and extracted with ethyl acetate (15 mL*3). The organic layer was washed with brine, dried, concentrated and the resultant residue was subjected to column chromatography on silica gel (dichloromethane:methanol=20:1) to give 4-(9-ethyl-2-(4-methoxy-3-(1-methyl-1H-pyrazol-3-yl)phenyl)-9H-purin-6-yl)morpholine (0.5 g, 84%) as a yellow solid. LCMS (ESI) m/z: 420.1 [M+H]⁺.

### Step 4: Synthesis of 4-(9-ethyl-8-iodo-2-(4-methoxy-3-(1-methyl-1H-pyrazol-3-yl)phenyl)-9H-purin-6-yl)morpholine

n-Butyllithium (2.5 M in tetrahydrofuran, 0.9 mL, 2.36 mmol) was added to a solution of 4-(9-ethyl-2-(4-methoxy-3-(1-methyl-1H-pyrazol-3-yl)phenyl)-9H-purin-6-yl)morpholine (900 mg, 2.15 mmol) in anhydrous tetrahydrofuran (15 mL) at −78° C. under nitrogen atmosphere. The reaction mixture was stirred at −78° C. for 1 hour, and Iodine (650 mg, 2.57 mmol) was added to the above solution at −78° C. under nitrogen atmosphere. The mixture reaction was stirred further at −78° C. for 1.5 h. It was quenched by adding aqueous ammonium chloride solution (10 mL) and extracted with ethyl acetate (20 mL*3). The combined organic layer was washed with brine (20 mL), dried over sodium sulphate, filtered and concentrated. The residue was subjected to column chromatography on silica gel (dichloromethane:methanol=10:1) to obtain 4-(9-ethyl-8-iodo-2-(4-methoxy-3-(1-methyl-1H-pyrazol-3-yl)phenyl)-9H-purin-6-yl)morpholine (500 mg, 43%) as a yellow solid. LCMS (ESI) m/z: 546.1 [M+H]⁺.

### Step 5: Synthesis of ethyl 2-(9-ethyl-2-(4-methoxy-3-(1-methyl-1H-pyrazol-3-yl)phenyl)-6-morpholino-9H-purin-8-yl)acetate

To a solution of 4-(9-ethyl-8-iodo-2-(4-methoxy-3-(1-methyl-1H-pyrazol-3-yl)phenyl)-9H-purin-6-yl)morpholine (0.2 g, 0.37 mmol), tris(dibenzylideneacetone)dipalladium (37 mg, 0.04 mmol) and 4,5-bis(diphenylphosphino)-9,9-dimethylxanthene (46 mg, 0.08 mmol) in tetrahydrofuran (10 mL) was added (2-ethoxy-2-oxoethyl)zinc(II) bromide solution (2.5 mL, 2.5 mmol) drop-wise at room temperature under argon atmosphere. After the addition, the reaction mixture was heated to 60° C. and stirred for 16 h. The reaction mixture was cooled down, quenched with aqueous ammonium chloride solution and extracted with ethyl acetate (20 mL×2). The combined organic phase was washed with brine (20 mL), dried over sodium sulfate, filtered and concentrated. The residue was purified by flash chromatography (eluted with ethyl acetate in petroleum ether from 20% to 40%) to obtain ethyl 2-(9-ethyl-2-(4-methoxy-3-(1-methyl-1H-pyrazol-3-yl)phenyl)-6-morpholino-9H-purin-8-yl)acetate (0.1 g, 54%) as yellow solid. LCMS (ESI) m/z: 506 [M+H]⁺.

### Step 6: Synthesis of 2-(9-ethyl-2-(4-methoxy-3-(1-methyl-1H-pyrazol-3-yl)phenyl)-6-morpholino-9H-purin-8-yl)ethan-1-ol

A solution of lithium aluminum hydride (1.0M, 0.41 mL, 0.41 mmol) in THE was added to a solution of ethyl 2-(9-ethyl-2-(4-methoxy-3-(1-methyl-1H-pyrazol-3-yl)phenyl)-6-morpholino-9H-purin-8-yl)acetate (100 mg, 0.2 mmol) in anhydrous tetrahydrofuran (5 mL) at 0° C. drop-wise. After the addition the reaction was stirred for 1.5 h at 0° C. under nitrogen atmosphere. It was then quenched by carefully adding sodium sulfate decahydrate with ice-bath cooling. Tetrahydrofuran (50 mL) was added to the reaction mixture and stirred for 15 min. The solids were filtered-off and the filtrate was concentrated. The residue was subjected to prep-HPLC (BOSTON pHlex ODS 10 μm 21.2×250 mm 120 A, with mobile phase acetonitrile/0.1% ammonium bicarbonate) to obtain 2-(9-ethyl-2-(4-methoxy-3-(1-methyl-1H-1 pyrazol-3-yl)phenyl)-6-morpholino-9H-purin-8-yl)ethan-1-ol (40.9 mg, 43%) as an off-white solid. ¹H NMR (400 MHz, DMSO-d₆) δ 8.83 (d, J=2.3 Hz, 1H), 8.30 (dd, J=8.7, 2.3 Hz, 1H), 7.72 (d, J=2.1 Hz, 1H), 7.16 (d, J=8.8 Hz, 1H), 6.70 (d, J=2.2 Hz, 1H), 4.84 (s, 1H), 4.30 (d, J=7.1 Hz, 2H), 4.28 (s, 4H), 3.92 (s, 3H), 3.90 (s, 3H), 3.83 (s, 2H), 3.79-3.74 (m, 4H), 3.02 (t, J=6.6 Hz, 2H), 1.38 (t, J=7.2 Hz, 3H). LCMS (ESI) m/z: 464.3 [M+H]⁺.

### Synthesis of (2-(3-methoxy-4-phenyl-1H-pyrazol-1-yl)-4-morpholino-7,8-dihydro-6H-pyrrolo[2,1-e]purin-6-yl)methanol (Compound 427)

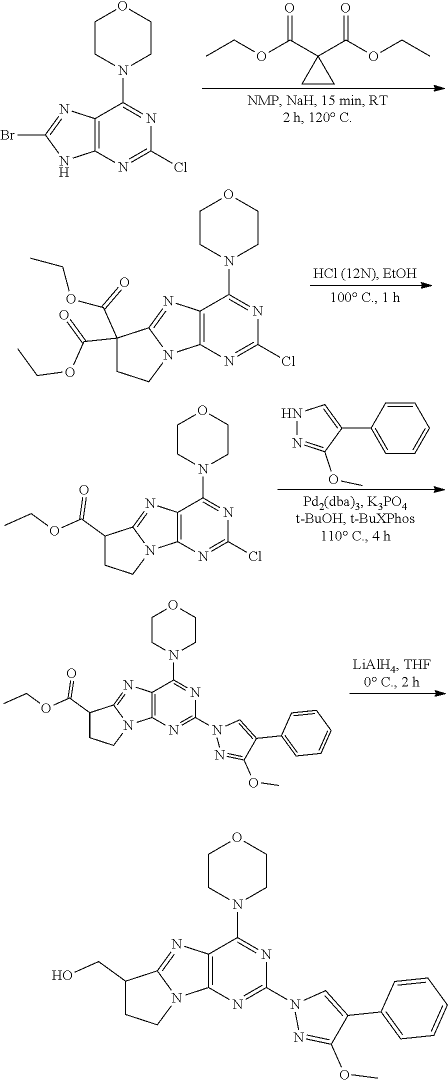

### Step 1: Synthesis of diethyl 2-chloro-4-morpholino-7,8-dihydro-6H-pyrrolo[2,1-e]purine-6,6-dicarboxylate

To a solution of 4-(8-bromo-2-chloro-9H-purin-6-yl)morpholine (827 mg, 2.6 mmol) in N-methyl pyrrolidone (4 mL) was added sodium hydride (60% suspension in mineral oil, 124 mg, 3.12 mmol) at room temperature. After stirring for 15 min, diethyl cyclopropane-1,1-dicarboxylate (604 mg, 3.25 mmol) was added to the reaction mixture. The resultant reaction was stirred at 120° C. for 2 h and was quenched by a small amount of water. The resultant mixture was then extracted with ethyl acetate (100 mL), washed with brine (100 mL×3), dried over sodium sulfate, filtered and concentrated. The crude product was purified by silica gel column chromatography (ethyl acetate/petroleum=0%-50%) to afford diethyl 2-chloro-4-morpholino-7,8-dihydro-6H-pyrrolo[2,1-e]purine-6,6-dicarboxylate (210 mg, 19%) as a yellow solid. LCMS (ESI) m/z: 423.7 [M+H]⁺.

### Step 2: Synthesis of ethyl 2-chloro-4-morpholino-7,8-dihydro-6H-pyrrolo[2,1-e]purine-6-carboxylate

A mixture of diethyl 2-chloro-4-morpholino-7,8-dihydro-6H-pyrrolo[2,1-e]purine-6,6-dicarboxylate (126 mg, 0.3 mmol) in concentrated hydrochloric acid (3 mL, 12N) and ethanol (6 mL) was stirred at 100° C. for 1 h. The mixture was concentrated and the residue was diluted with water (80 mL), the pH was adjusted to 8 with saturated aqueous sodium bicarbonate solution and extracted with dichloromethane (100 mL×2). The organic phase was washed with brine (100 mL), dried over sodium sulfate, filtered and concentrated to afford ethyl 2-chloro-4-morpholino-7,8-dihydro-6H-pyrrolo[2,1-e]purine-6-carboxylate (98 mg, 92%) as a yellow solid. LCMS (ESI) m/z: 351.8 [M+H]⁺.

### Step 3: Synthesis of ethyl 2-(3-methoxy-4-phenyl-1H-pyrazol-1-yl)-4-morpholino-7,8-dihydro-6H-pyrrolo[2,1-e]purine-6-carboxylate

A mixture of ethyl 2-chloro-4-morpholino-7,8-dihydro-6H-pyrrolo[2,1-e]purine-6-carboxylate (125 mg, 0.356 mmol), 3-methoxy-4-phenyl-1H-pyrazole (74 mg, 0.427 mmol), tris(dibenzylideneacetone)dipalladium(0) (33 mg, 0.036 mmol), potassium phosphate (151 mg, 0.712 mmol) and 2-di-tert-butylphosphino-2′,4′,6′-trisopropylbinphenyl (30 mg, 0.071 mmol) in tert-butanol (10 mL) was stirred at 110° C. for 4 h under argon protection. The mixture was concentrated and the residue was subjected to prep-HPLC (BOSTON pHlex ODS 10 μm 21.2×250 mm 120 A. The mobile phase was acetonitrile/0.1% ammonium bicarbonate) to afford ethyl 2-(3-methoxy-4-phenyl-1H-pyrazol-1-yl)-4-morpholino-7,8-dihydro-6H-pyrrolo[2,1-e]purine-6-carboxylate (100 mg, 45%) as white solid. LCMS (ESI) m/z: 489.8 [M+H]⁺.

### Step 4: Synthesis of (2-(3-methoxy-4-phenyl-1H-pyrazol-1-yl)-4-morpholino-7,8-dihydro-6H-pyrrolo[2,1-e]purin-6-yl)methanol

To a solution of ethyl 2-(3-methoxy-4-phenyl-1H-pyrazol-1-yl)-4-morpholino-7,8-dihydro-6H-pyrrolo[2,1-e]purine-6-carboxylate (100 mg, 0.2 mmol) in anhydrous tetrahydrofuran (10 mL) was added a solution of lithium aluminum hydride (1.0M in THF, 0.6 mL, 0.6 mmol) at 0° C. and the resultant mixture was stirred under nitrogen for 2 h. The reaction mixture was quenched carefully by the addition of sodium sulfate decahydrate with ice-bath cooling. The resultant solids were filtered-off with additional dichloromethane (20 mL) washings. The combined filtrates were concentrated and the residue was subjected to prep-HPLC (BOSTON pHlex ODS 10 μm 21.2×250 mm 120 A. The mobile phase was acetonitrile/0.1% ammonium bicarbonate) to obtain (2-(3-methoxy-4-phenyl-1H-pyrazol-1-yl)-4-morpholino-7,8-dihydro-6H-pyrrolo[2,1-e]purin-6-yl)methanol (17.6 mg, 19%) as a white solid. ¹H NMR (400 MHz, DMSO-d₆) δ 8.90 (s, 1H), 7.81 (d, J=7.2 Hz, 2H), 7.39 (t, J=7.7 Hz, 2H), 7.24 (t, J=7.4 Hz, 1H), 4.97 (t, J=5.3 Hz, 1H), 4.28 (bs, 4H), 4.16-4.08 (m, 2H), 4.06 (s, 3H), 3.81-3.74 (m, 5H), 3.72-3.67 (m, 1H), 3.31-3.27 (m, 1H), 2.82-2.69 (m, 1H), 2.60-2.53 (m, 1H). LCMS (ESI) m/z: 448.3[M+H]⁺.

### Synthesis of 4-(2-(3-methoxy-4-phenyl-1H-pyrazol-1-yl)-7,8-dihydro-6H-pyrrolo[2,1-e]purin-4-yl)morpholine (Compound 428)

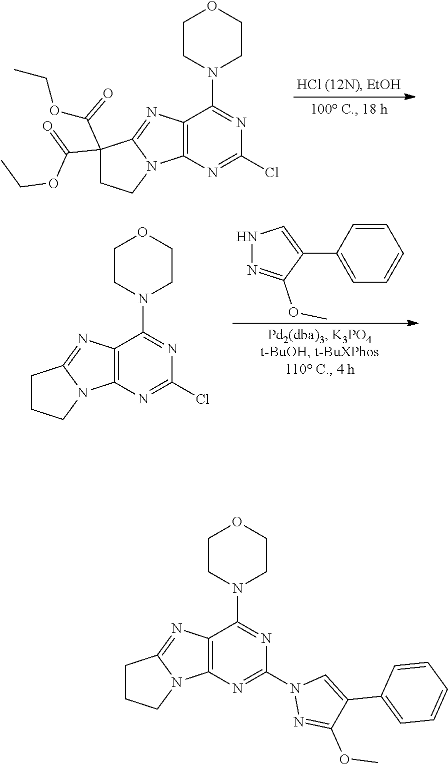

### Step 1: Synthesis of 4-(2-chloro-7,8-dihydro-6H-pyrrolo[2,1-e]purin-4-yl)morpholine

A solution of diethyl 2-chloro-4-morpholino-7,8-dihydro-6H-pyrrolo[2,1-e]purine-6,6-dicarboxylate (58 mg, 0.137 mmol) in concentrated hydrochloric acid (3 mL, 12N) and ethanol (6 mL) was stirred at 100° C. for 18 h. The mixture was concentrated and the residue was subjected to prep-HPLC (BOSTON pHlex ODS 10 μm 21.2×250 mm 120 A. The mobile phase was acetonitrile/0.1% ammonium bicarbonate) to afford 4-(2-chloro-7,8-dihydro-6H-pyrrolo[2,1-e]purin-4-yl)morpholine (22 mg, 57%) as a white solid. LCMS (ESI) m/z: 280.0 [M+H]⁺.

### Step 2: Synthesis of 4-(2-(3-methoxy-4-phenyl-1H-pyrazol-1-yl)-7,8-dihydro-6H-pyrrolo[2,1-e]purin-4-yl)morpholine

A mixture of 4-(2-chloro-7,8-dihydro-6H-pyrrolo[2,1-e]purin-4-yl)morpholine (17 mg, 0.06 mmol), 3-methoxy-4-phenyl-1H-pyrazole (13 mg, 0.072 mmol), tris(dibenzylideneacetone)dipalladium(0) (6 mg, 0.006 mmol), potassium phosphate (25 mg, 0.12 mmol) and 2-di-tert-butylphosphino-2′,4′,6′-trisopropylbinphenyl (5 mg, 0.012 mmol) in tert-butanol (2 mL) was stirred at 110° C. for 4 h under argon protection. The mixture was concentrated and the residue was subjected to prep-HPLC (BOSTON pHlex ODS 10 μm 21.2×250 mm 120 A. The mobile phase was acetonitrile/0.1% ammonium bicarbonate) to afford 4-(2-(3-methoxy-4-phenyl-1H-pyrazol-1-yl)-7,8-dihydro-6H-pyrrolo[2,1-e]purin-4-yl)morpholine (8.9 mg, 46%) as white solid. ¹H NMR (500 MHz, CDCl3) δ 8.60 (s, 1H), 7.74 (d, J=7.4 Hz, 2H), 7.39 (t, J=7.7 Hz, 2H), 7.24 (d, J=7.5 Hz, 1H), 4.33 (bs, 4H), 4.25 (t, J=7.1 Hz, 2H), 4.21 (s, 3H), 3.92-3.84 (m, 4H), 3.01 (t, J=7.6 Hz, 2H), 2.74-2.65 (m, 2H). LCMS (ESI) m/z: 418.2[M+H]⁺.

### Synthesis of enantiomer 1 (Compound 429) and enantiomer 2 (Compound 430) of (9-ethyl-2-(3-(1-methyl-1H-pyrazol-3-yl)phenyl)-6-morpholino-9H-purin-8-yl)(pyridin-4-yl)methanol

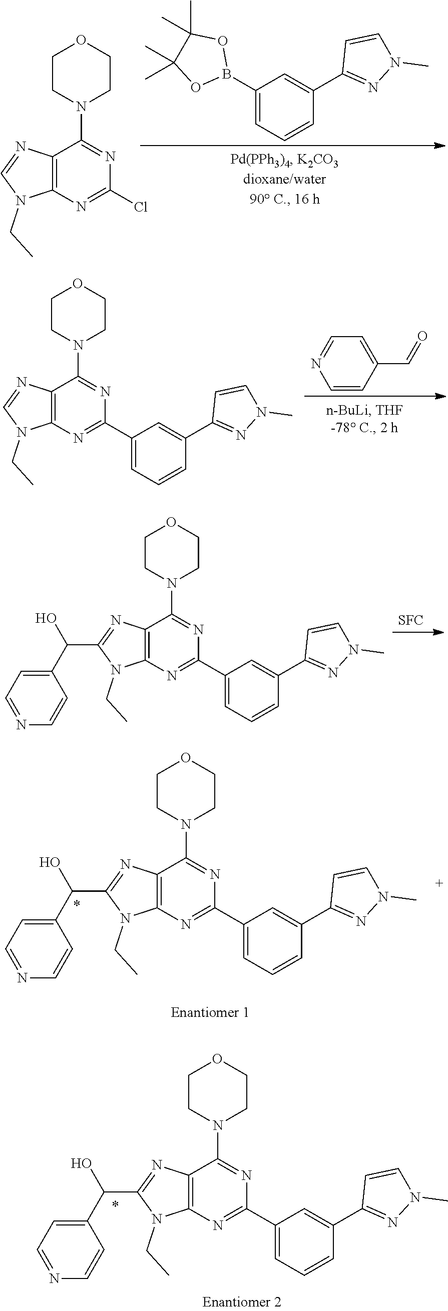

### Step 1: Synthesis of 4-(9-ethyl-2-(3-(1-methyl-1H-pyrazol-3-yl)phenyl)-9H-purin-6-yl)morpholine

To a solution of 4-(2-chloro-9-ethyl-9H-purin-6-yl)morpholine (1.2 g, 4.5 mmol) and 1-methyl-3-(3-(4,4,5,5-tetramethyl-1,3,2-dioxaborolan-2-yl)phenyl)-1H-pyrazole (1.6 g, 11.2 mmol) in dioxane/water (25 mL/8 mL) were added potassium carbonate (1.6 g, 11.2 mmol) and tetrakis(triphenylphosphine)palladium (0.5 g, 0.5 mmol). The resultant mixture was stirred at 90° C. for 16 h, then poured into ice-water and extracted with ethyl acetate (15 mL*3). The combined organic layer was washed with brine, dried over sodium sulfate and evaporated to dryness. The crude product was chromatographed on silica gel (dichloromethane:methanol=20:1) to obtain 4-(9-ethyl-2-(3-(1-methyl-1H-pyrazol-3-yl)phenyl)-9H-purin-6-yl)morpholine (1.3 g, 74%) as a yellow solid. LCMS (ESI) m/z: 390.0 [M+H]⁺.

### Step 2: Synthesis and separation of Compound 91 and Compound 92

n-Butyllithium (2.5 M in tetrahydrofuran, 1.2 mL, 3.1 mmol) was added to a solution of 4-(9-ethyl-2-(3-(1-methyl-1H-pyrazol-3-yl)phenyl)-9H-purin-6-yl)morpholine (600 mg, 1.54 mmol) in anhydrous tetrahydrofuran (40 mL) at −78° C. under nitrogen atmosphere. The reaction mixture was stirred at −78° C. for 1 h, and isonicotinaldehyde (330 mg, 3.1 mmol) in tetrahydrofuran (1.0 mL) was added to the above solution at −78° C. under nitrogen atmosphere. The resultant mixture reaction was warmed up and stirred at 25° C. for 1.5 h. It was quenched by adding aqueous ammonium chloride solution (10 mL) and extracted with ethyl acetate (20 mL*3). The combined organic layer was washed with brine (20 mL), dried over sodium sulphate, filtered and concentrated. The residue was purified by HPLC (BOSTON pHlex ODS 10 μm 21.2×250 mm 120 A, with mobile phase acetonitrile/0.1% ammonium bicarbonate) to afford the racemic (9-ethyl-2-(3-(1-methyl-1H-pyrazol-3-yl)phenyl)-6-morpholino-9H-purin-8-yl)(pyridin-4-yl)methanol (180 mg) as a yellow solid. This achiral compound was subjected to chiral prep-HPLC (Instrument: SFC-80 (Thar, Waters), Column: AD 20*250 mm, 10 um (Daicel), Column temperature: 35° C., Mobile phase: carbon dioxide/ethanol (0.5% methanol ammonia)=65/35, Flow rate: 80 g/min, Back pressure: 100 bar, Detection wavelength: 214 nm, Cycle time: 3.0 min, Sample solution: 200 mg dissolved in 25 mL Methanol, Injection volume: 1.0 mL) to obtain the two enantiomers: compound 91 (45.2 mg, 6%) and compound 92 (34.8 mg, 5%) respectively.

Compound 91: ¹H NMR (400 MHz, DMSO-d₆) δ 8.72 (s, 1H), 8.58 (dd, J=4.5, 1.5 Hz, 2H), 8.29 (d, J=7.9 Hz, 1H), 7.84 (d, J=7.7 Hz, 1H), 7.77 (d, J=2.2 Hz, 1H), 7.48 (t, J=7.7 Hz, 1H), 7.42 (d, J=5.5 Hz, 2H), 6.94 (d, J=5.0 Hz, 1H), 6.72 (d, J=2.2 Hz, 1H), 6.12 (d, J=4.9 Hz, 1H), 4.42-4.27 (m, 4H), 4.17 (q, J=7.1 Hz, 2H), 3.91 (s, 3H), 3.85-3.72 (m, 4H), 1.15 (t, J=7.1 Hz, 3H). LCMS (ESI) m/z: 497.1 [M+H]⁺; (RT: 0.982 min).

Compound 92: ¹H NMR (400 MHz, DMSO-d₆) δ 8.72 (s, 1H), 8.58 (dd, J=4.5, 1.5 Hz, 2H), 8.29 (d, J=7.9 Hz, 1H), 7.84 (d, J=7.7 Hz, 1H), 7.77 (d, J=2.2 Hz, 1H), 7.48 (t, J=7.7 Hz, 1H), 7.42 (d, J=5.5 Hz, 2H), 6.94 (d, J=5.0 Hz, 1H), 6.72 (d, J=2.2 Hz, 1H), 6.12 (d, J=4.9 Hz, 1H), 4.42-4.27 (m, 4H), 4.15 (q, J=7.1 Hz, 2H), 3.91 (s, 3H), 3.85-3.72 (m, 4H), 1.15 (t, J=7.1 Hz, 3H). LCMS (ESI) m/z: 497.1 [M+H]⁺; (RT: 1.882 min).

### Synthesis of 4-(8-bromo-9-ethyl-2-(3-methoxy-4-phenyl-1H-pyrazol-1-yl)-9H-purin-6-yl)morpholine (Compound 431)

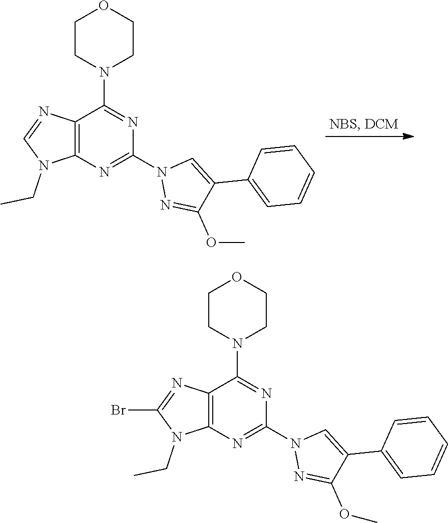

To a solution of 4-(9-ethyl-2-(3-methoxy-4-phenyl-1H-pyrazol-1-yl)-9H-purin-6-yl)morpholine (80 mg, 0.198 mmol) in dichloromethane (10 mL) was added NBS (35 mg, 0.198 mmol). The reaction mixture was stirred at 0° C. for 4 h, then the reaction was quenched with water (15 mL) and extracted with ethyl acetate (20*3 mL). The organic layer was combined, washed with brine (30 mL), dried over sodium sulfate, filtered and concentrated. The residue was subjected to prep-HPLC (0.05% ammonium bicarbonate:acetonitrile=5%-95%) to obtain 4-(8-bromo-9-ethyl-2-(3-methoxy-4-phenyl-1H-pyrazol-1-yl)-9H-purin-6-yl)morpholine (18.0 mg, 19%) as a white solid. ¹H NMR (400 MHz, DMSO-d₆) δ 8.93 (s, 1H), 7.81 (dd, J=8.2, 1.1 Hz, 2H), 7.40 (t, J=7.8 Hz, 2H), 7.25 (t, J=7.4 Hz, 1H), 4.35-4.05 (m, 6H), 4.07 (s, 3H), 3.85-3.70 (m, 4H), 1.37 (t, J=7.2 Hz, 3H). LCMS (ESI) m/z: 485.5 [M+H]⁺.

### Synthesis of 1-(9-ethyl-2-(3-methoxy-4-phenyl-1H-pyrazol-1-yl)-9H-purin-6-yl)pyrrolidin-2-one (Compound 432)

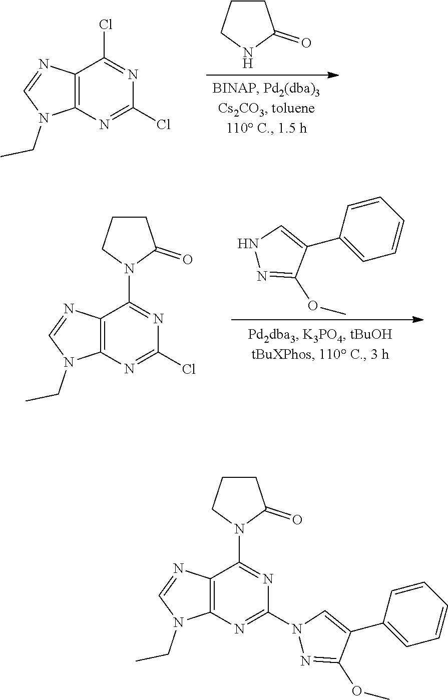 [SMILES: CCn1cnc2c(Cl)nc(Cl)nc21 ; O=C1CCCN1 ; CCn1cnc2c(N3CCCC3=O)nc(Cl)nc21 ; COc1n[nH]cc1-c1ccccc1 ; CCn1cnc2c(N3CCCC3=O)nc(-n3cc(-c4ccccc4)c(OC)n3)nc21]

### Step 1: Synthesis of 1-(2-chloro-9-ethyl-9H-purin-6-yl)pyrrolidin-2-one

A mixture of 2,6-dichloro-9-ethyl-9H-purine (1 g, 4.6 mmol), pyrrolidin-2-one (313 mg, 3.7 mmol) 1.1′-binaphthyl-2.2′-diphemyl phosphine (572 mg, 0.92 mmol), tris(dibenzylideneacetone)dipalladium (420 mg, 0.46 mmol) and cesium carbonate (30 mg, 0.22 mmol) in toluene (10 mL) was stirred at 110° C. for 1.5 h. The mixture was then filtered through a pad of celite and the filtrate was subjected to prep-HPLC (Column Xbridge 21.2*250 mm C18, 10 um, Mobile Phase A: water (10 mmol/L ammonium bicarbonate) B: acetonitrile) to obtain 1-(2-chloro-9-ethyl-9H-purin-6-yl)pyrrolidin-2-one (350 mg, 28%) as a white solid. LCMS (ESI) m/z: 265.8 [M+H]⁺.

### Step 2: Synthesis of 1-(9-ethyl-2-(3-methoxy-4-phenyl-1H-pyrazol-1-yl)-9H-purin-6-yl)pyrrolidin-2-one

A mixture of 1-(2-chloro-9-ethyl-9H-purin-6-yl)pyrrolidin-2-one (50 mg, 0.18 mmol), 3-methoxy-4-phenyl-1H-pyrazole (50 mg, 0.28 mmol), potassium phosphate (57.24 mg, 0.27 mmol), ditert-butyl-[2-[2,4,6-tri(propan-2-yl)phenyl]phenyl]phosphane (16.96 mg, 0.04 mmol) and tris(dibenzylideneacetone)dipalladium (18.3 mg, 0.02 mmol) in tert-butanol (5 mL) was stirred at 110° C. for 3 h. The mixture was filtered through a pad of celite and the resultant filtrate was subjected to prep-HPLC (Column Xbridge 21.2*250 mm C18, 10 um, Mobile Phase A: water (10 mmol/L ammonium bicarbonate) B: acetonitrile) to obtain 1-(9-ethyl-2-(3-methoxy-4-phenyl-1H-pyrazol-1-yl)-9H-purin-6-yl)pyrrolidin-2-one (32.8 mg, 45%) as a white solid. ¹H NMR (400 MHz, DMSO-d₆) δ 8.92 (s, 1H), 8.48 (s, 1H), 7.78 (d, J=7.2 Hz, 2H), 7.42 (t, J=7.7 Hz, 2H), 7.27 (s, 1H), 4.35-4.25 (m, 4H), 4.10 (s, 3H), 2.60 (s, 2H), 2.22-2.13 (m, 2H), 1.50 (t, J=7.3 Hz, 3H). LCMS (ESI) m/z: 404.1 [M+H]⁺.

### Synthesis of 1-(9-ethyl-2-(3-(1-methyl-1H-pyrazol-3-yl)phenyl)-9H-purin-6-yl)pyrrolidin-2-one (Compound 433)

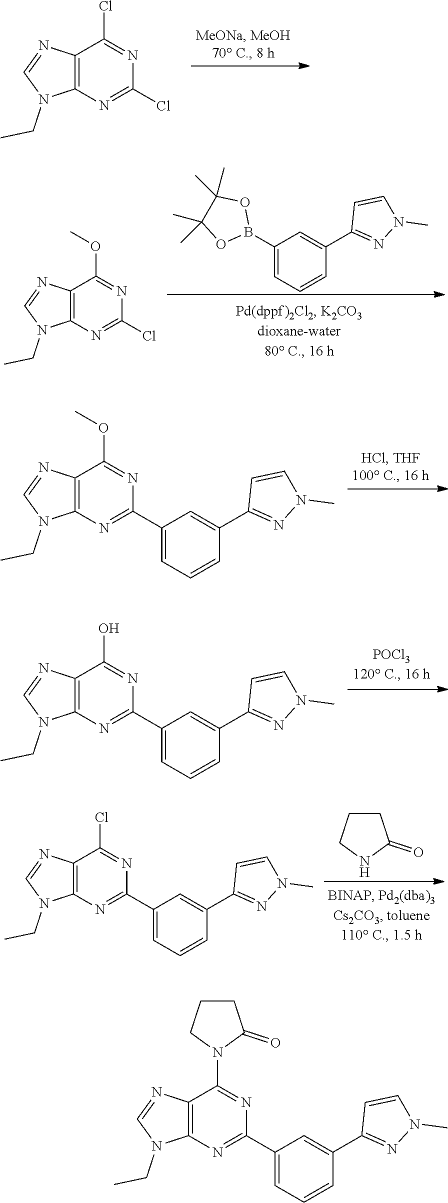

### Step 1: Synthesis of 2-chloro-9-ethyl-6-methoxy-9H-purine

A mixture of 2,6-dichloro-9-ethyl-9H-purine (500 mg, 2.3 mmol) and sodium methanolate (414 mg, 2.3 mmol) in methanol (5 mL) was stirred at 70° C. for 2 h. Then the reaction was quenched by the addition of the water and extracted with ethyl acetate (100 mL). The organic layer was washed with water, dried over sodium sulfate and evaporated to dryness. The crude product was chromatographed on silica gel (petroleum ether/ethyl acetate 10:1) to obtain 2-chloro-9-ethyl-6-methoxy-9H-purine (400 mg, 82%) as a white solid. LCMS (ESI) m/z: 212.9 [M+H]⁺.

### Step 2: Synthesis of 9-ethyl-6-methoxy-2-(3-(1-methyl-1H-pyrazol-3-yl)phenyl)-9H-purine

A mixture of 2-chloro-9-ethyl-6-methoxy-9H-purine (400 mg, 1.88 mmol), 1-methyl-3-(3-(4,4,5,5-tetramethyl-1,3,2-dioxaborolan-2-yl)phenyl)-1H-pyrazole (803 mg, 2.83 mmol), 1,1′-bis(diphenylphosphino)ferrocene-palladium(II)dichloride dichloromethane (137 mg, 0.19 mmol) and potassium carbonate (778 mg, 5.64 mmol) in 1,4-dioxane (5 mL) and water (0.5 mL) was stirred at 80° C. for 16 h. The resultant mixture was filtered through a pad of celite and the filtrate was concentrated. The crude product was chromatographed on silica gel (petroleum ether/ethyl acetate 8:1) to obtain 9-ethyl-6-methoxy-2-(3-(1-methyl-1H-pyrazol-3-yl)phenyl)-9H-purine (550 mg, 87%) as a white solid. LCMS (ESI) m/z: 335.2 [M+H]⁺.

### Step 2: Synthesis of 9-ethyl-2-(3-(1-methyl-1H-pyrazol-3-yl)phenyl)-9H-purin-6-ol

A mixture of 9-ethyl-6-methoxy-2-(3-(1-methyl-1H-pyrazol-3-yl)phenyl)-9H-purine (550 mg, 1.64 mmol) in hydrochloric acid (1 mL, 12 mol/L) and tetrahydrofuran was stirred at 120° C. for 16 h. Ethyl acetate (100 mL) and saturated aqueous sodium bicarbonate solution (50 mL) were added to the residue, The organic layer was washed with water, dried and concentrated to give 9-ethyl-2-(3-(1-methyl-1H-pyrazol-3-yl)phenyl)-9H-purin-6-ol (400 mg, 76%) as a white solid. LCMS (ESI) m/z: 321.1 [M+H]⁺.

### Step 3: Synthesis of 6-chloro-9-ethyl-2-(3-(1-methyl-1H-pyrazol-3-yl)phenyl)-9H-purine

A mixture of 9-ethyl-2-(3-(1-methyl-1H-pyrazol-3-yl)phenyl)-9H-purin-6-ol (320 mg, 1 mmol) in phosphorus oxychloride (5 mL) was stirred at 120° C. for 16 h. The reaction mixture was added dropwise to water (100 mL), and then neutralized with 5 M aqueous sodium hydroxide solution. The mixture was extracted with ethyl acetate (50 mL×2), the combined organic layer was washed with brine (30 mL), dried over sodium sulfate, filtered and concentrated. The resulting residue was subjected to silica gel column chromatography (n-hexane/ethyl acetate=10/1) to obtain 6-chloro-9-ethyl-2-(3-(1-methyl-1H-pyrazol-3-yl)phenyl)-9H-purine (250 mg, 74%) as a white solid. LCMS (ESI) m/z: 338.7 [M+H]⁺.

### Step 4: Synthesis of 1-(9-ethyl-2-(3-(1-methyl-1H-pyrazol-3-yl)phenyl)-9H-purin-6-yl)pyrrolidin-2-one

A mixture of 6-chloro-9-ethyl-2-(3-(1-methyl-1H-pyrazol-3-yl)phenyl)-9H-purine (40 mg, 0.12 mmol) and pyrrolidin-2-one (15 mg, 0.17 mmol)m cesium carbonate (78 mg, 0.24 mmol) 1.1′-binaphthyl-2.2′-diphemyl phosphine (14 mg, 0.02 mmol) and tris(dibenzylideneacetone)dipalladium (30 mg, 0.22 mmol) in toluene (5 mL) was stirred at 110° C. for 1.5 h. The mixture was then filtered through a pad of celite and the filtrate was subjected to prep-HPLC (Column Xbridge 21.2*250 mm C18, 10 um, Mobile Phase A: water (10 mmol/L ammonium bicarbonate) B: acetonitrile) to obtain 1-(9-ethyl-2-(3-(1-methyl-1H-pyrazol-3-yl)phenyl)-9H-purin-6-yl)pyrrolidin-2-one (6.5 mg, 14%) as a white solid. ¹H NMR (400 MHz, DMSO-d₆) δ 8.86 (s, 1H), 8.54 (s, 1H), 8.39 (d, J=7.9 Hz, 1H), 7.89 (d, J=7.7 Hz, 1H), 7.79 (d, J=2.2 Hz, 1H), 7.54 (t, J=7.7 Hz, 1H), 6.76 (d, J=2.2 Hz, 1H), 4.39 (q, J=7.3 Hz, 2H), 4.30 (t, J=7.0 Hz, 2H), 3.93 (s, 3H), 2.60 (d, J=8.1 Hz, 2H), 2.20 (s, 2H), 1.53 (t, J=7.3 Hz, 3H). LCMS (ESI) m/z: 387.8 [M+H]⁺.

### Synthesis of 4-(8-(difluoromethyl)-9-ethyl-2-(3-methoxy-4-phenyl-1H-pyrazol-1-yl)-9H-purin-6-yl)morpholine (Compound 434)

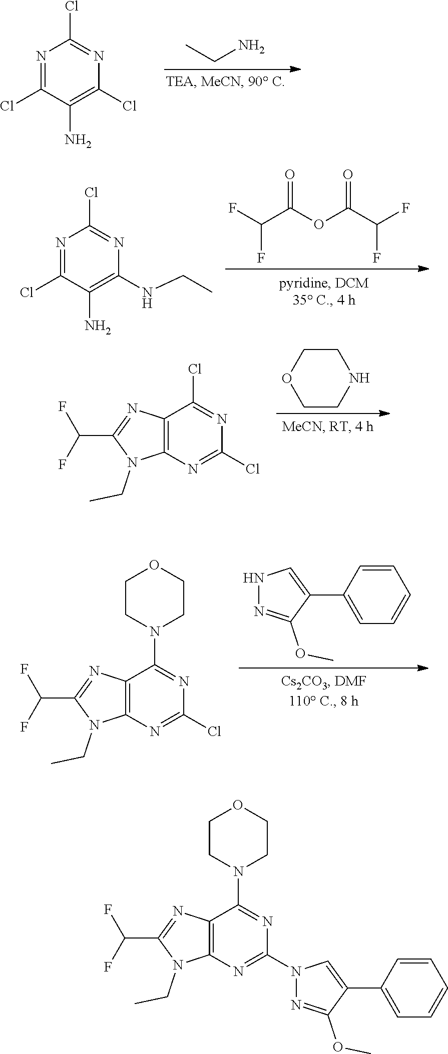

### Step 1: Synthesis of 2,6-dichloro-N4-ethylpyrimidine-4,5-diamine

To a solution of 2,4,6-trichloropyrimidin-5-amine (600 mg, 3.03 mol) in acetonitrile (10 mL) was added ethanamine (272 mg, 6.606 mmol) and the mixture was stirred at 90° C. for 4 h. Then the reaction was quenched with water (100 mL) and extracted with ethyl acetate (50 mL*3). The organic layers were combined, washed with brine (100 mL), dried over sodium sulfate, filtered and concentrated. The residue was subjected to flash chromatography on silica gel (petroleum ether:ethyl acetate=75:25) to obtain 2,6-dichloro-N4-ethylpyrimidine-4,5-diamine as yellow solid. (720 mg, 100% yield). LCMS (ESI) m/z: 207.1 [M+H]⁺.

### Step 2: Synthesis of 2,6-dichloro-8-(difluoromethyl)-9-ethyl-9H-purine

To a solution of 2,6-dichloro-N4-ethylpyrimidine-4,5-diamine (540 mg, 2.61 mmol) in dichloromethane (10 mL) were added 2,2-difluoroacetic anhydride (905 mg, 5.22 mmol) and pyridine (2 mL.). The resultant mixture was stirred at 35° C. for 4 h, then quenched with water (15 mL) and extracted with ethyl acetate (15 mL*3). The organic layers were combined, washed with brine (30 mL), dried over sodium sulfate, filtered and concentrated. The residue was subjected to flash chromatography on silica gel (petroleum ether:ethyl acetate=75:25) to obtain 2,6-dichloro-8-(difluoromethyl)-9-ethyl-9H-purine as yellow solid. (170 mg, 24.5%). LCMS (ESI) m/z: 267.1 [M+H]⁺.

### Step 3: Synthesis of 4-(2-chloro-8-(difluoromethyl)-9-ethyl-9H-purin-6-yl)morpholine

To a solution of 2,6-dichloro-8-(difluoromethyl)-9-ethyl-9H-purine (170 mg, 0.637 mmol) in acetonitrile (10 mL) was added morpholine (110 mg, 1.273 mmol). The reaction mixture was stirred at 30° C. for 4 h, then quenched with water (10 mL) and extracted with ethyl acetate (15 mL*3). The organic layers were combined, washed with brine (50 mL), dried over sodium sulfate, filtered and concentrated.

The residue was purified by flash chromatography on silica gel (petroleum ether:ethyl acetate=75:25) to obtain 4-(2-chloro-8-(difluoromethyl)-9-ethyl-9H-purin-6-yl)morpholine as yellow solid. (160 mg, 79.2%). LCMS (ESI) m/z: 237.9[M+H]⁺.

### Step 4: Synthesis of 4-(8-(difluoromethyl)-9-ethyl-2-(3-methoxy-4-phenyl-1H-pyrazol-1-yl)-9H-purin-6-yl)morpholine

To a solution of 4-(2-chloro-8-(difluoromethyl)-9-ethyl-9H-purin-6-yl)morpholine (160 mg, 0.504 mmol) in N,N-dimethylformamide (5 mL) were added 3-methoxy-4-phenyl-1H-pyrazole (175 mg, 1.01 mmol) and cesium carbonate (32 mg, 1.0 mmol). The mixture was stirred at 110° C. for 8 h, then quenched with water (15 mL) and extracted with ethyl acetate (20 mL*3). The organic layers were combined, washed with brine (30 mL), dried over sodium sulfate, filtered, and concentrated. The residue was subjected to prep-HPLC (0.05% ammonium bicarbonate:acetonitrile=5%-95%) to afford 4-(8-(difluoromethyl)-9-ethyl-2-(3-methoxy-4-phenyl-1H-pyrazol-1-yl)-9H-purin-6-yl)morpholine (74.2 mg, 32.3%) as a white solid. ¹H NMR (400 MHz, DMSO-d₆) δ 8.96 (s, 1H), 7.82 (d, J=7.9 Hz, 2H), 7.50-7.34 (m, 3H), 7.30-7.22 (m, 1H), 4.45-4.25 (m, 6H), 4.08 (s, 3H), 3.84-3.75 (m, 4H), 1.42 (t, J=7.1 Hz, 3H). LCMS (ESI) m/z: 456.1[M+H]⁺.

### Synthesis of 4-(8-ethyl-2-(3-methoxy-4-phenyl-1H-pyrazol-1-yl)-9-methyl-9H-purin-6-yl)morpholine (Compound 435)

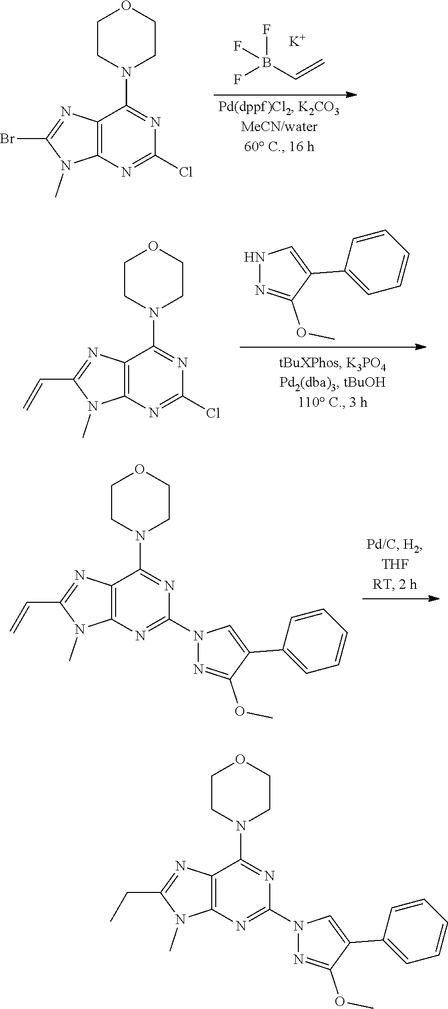

### Step 1: Synthesis of 4-(2-chloro-9-methyl-8-vinyl-9H-purin-6-yl)morpholine

A mixture of 4-(8-bromo-2-chloro-9-methyl-9H-purin-6-yl)morpholine (664 mg, 2 mmol), vinylboronic acid trifluoroborate potassium salt (252 mg, 1.9 mmol), potassium carbonate (828 mg, 6 mmol) and [1,1′-bis(diphenylphosphino)ferrocene]dichloro palladium(II) (146 mg, 0.2 mmol) in acetonitrile (5 mL) and water (0.7 mL) was stirred at 60° C. under argon for 16 h. The mixture was filtered through a pad of celite, and the filtrate was concentrated to give the crude product. The crude product was then chromatographed on silica gel (petroleum ether/ethyl acetate 8:1) to give 4-(2-chloro-9-methyl-8-vinyl-9H-purin-6-yl)morpholine (550 mg, 98%) as a white solid. LCMS (ESI) m/z: 279.8 [M+H]⁺.

### Step 2: Synthesis of 4-(2-(3-methoxy-4-phenyl-1H-pyrazol-1-yl)-9-methyl-8-vinyl-9H-purin-6-yl)morpholine

A mixture of 4-(2-chloro-9-methyl-8-vinyl-9H-purin-6-yl)morpholine (100 mg, 0.35 mmol), 3-methoxy-4-phenyl-1H-pyrazole (93 mg, 0.53 mmol), potassium phosphate tribasic (111 mg, 0.52 mmol), ditert-butyl-[2-[2,4,6-tri(propan-2-yl)phenyl]phenyl]phosphane (29 mg, 0.07 mmol) and tris(dibenzylideneacetone)dipalladium (32 mg, 0.035 mmol) in tert-butanol (3 mL) was stirred at 110° C. for 3 h. The mixture was filtered through a pad of celite and the filtrated was concentrated. The residue was then subjected to column chromatography on silica gel (petroleum ether/ethyl acetate 5:1) to obtain 4-(2-(3-methoxy-4-phenyl-1H-pyrazol-1-yl)-9-methyl-8-vinyl-9H-purin-6-yl)morpholine (120 mg, 82%) as a white solid. LCMS (ESI) m/z: 418.2 [M+H]⁺.

### Step 3: Synthesis of 4-(8-ethyl-2-(3-methoxy-4-phenyl-1H-pyrazol-1-yl)-9-methyl-9H-purin-6-yl)morpholine

A mixture of 4-(2-(3-methoxy-4-phenyl-1H-pyrazol-1-yl)-9-methyl-8-vinyl-9H-purin-6-yl)morpholine (150 mg, 0.72 mmol) and palladium on carbon catalyst (15 mg) in methanol (5 mL) under hydrogen atmosphere was stirred at room temperature for 16 h. The crude product was purified by prep-HPLC (Column Xbridge 21.2*250 mm C18, 10 um, Mobile Phase A: water (10 mmol/L ammonium bicarbonate) B: acetonitrile) to obtain 4-(8-ethyl-2-(3-methoxy-4-phenyl-1H-pyrazol-1-yl)-9-methyl-9H-purin-6-yl)morpholine (97.8 mg, 64%) as a white solid. ¹H NMR (400 MHz, DMSO-d₆) δ 8.92 (s, 1H), 7.87-7.75 (m, 2H), 7.39 (t, J=7.7 Hz, 2H), 7.25 (d, J=7.4 Hz, 1H), 4.29 (s, 4H), 4.07 (s, 3H), 3.83-3.73 (m, 4H), 3.70 (s, 3H), 2.86 (d, J=7.5 Hz, 2H), 1.30 (t, J=7.5 Hz, 3H). LCMS (ESI) m/z: 420.1 [M+H]⁺

### Synthesis of 4-(2-(5-chloro-4-phenyl-1H-pyrazol-1-yl)-9-ethyl-9H-purin-6-yl)morpholine (Compound 436)

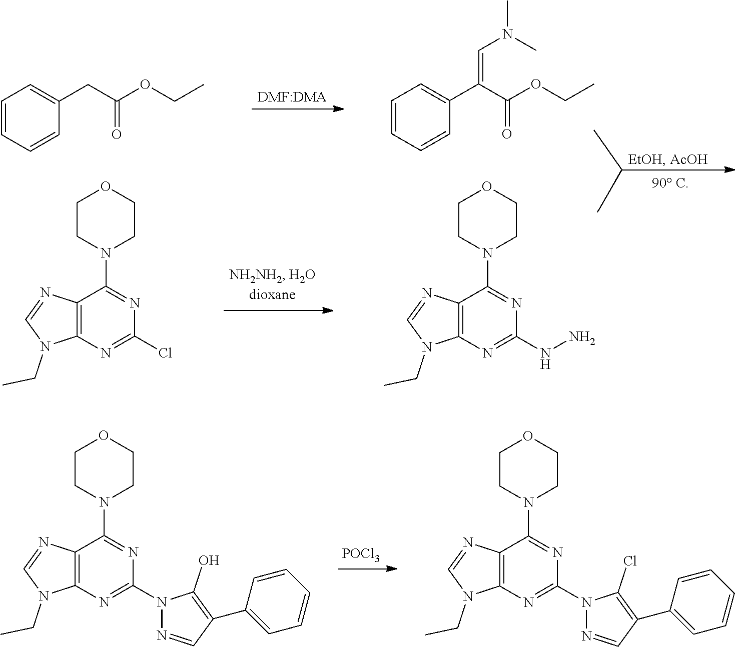

### Step 1: Synthesis of ethyl (Z)-3-(dimethylamino)-2-phenylacrylate

To a solution of ethyl 2-phenylacetate (20 g, 0.122 mol) in N,N-dimethylforamide dimethyl acetal (50 mL) was added DMF (10 mL). The reaction was stirred at 100° C. for 3 h and concentrated. It was then vacuum cried to obtain ethyl (Z)-3-(dimethylamino)-2-phenylacrylate (22 g, 50%) as a yellow oil. LCMS (ESI) m/z: 220.1 [M+H]⁺.

### Step 2: Synthesis of 4-(9-ethyl-2-hydrazineyl-9H-purin-6-yl)morpholine

A mixture of 4-(2-chloro-9-ethyl-9H-purin-6-yl)morpholine (267 mg, 1.0 mmol), hydrazine hydrate (1 mL) and 1,4-dioxane (12 mL) was stirred at 100° C. for 16 h. The mixture was concentrated to give 4-(9-ethyl-2-hydrazineyl-9H-purin-6-yl)morpholine (230 mg, 87.4%) as a yellow solid. LCMS (ESI) m/z: 264.1 [M+H]⁺.

### Step 3: Synthesis of 1-(9-ethyl-6-morpholino-9H-purin-2-yl)-4-phenyl-1H-pyrazol-5-ol

The mixture of 4-(9-ethyl-2-hydrazineyl-9H-purin-6-yl)morpholine (230 mg, 0.87 mmol) and ethyl (Z)-3-(dimethylamino)-2-phenylacrylate (380 mg, 1.74 mmol) in glacial acetic acid (10 mL) was stirred at 100° C. under nitrogen atmosphere for 11 h. Then the reaction was quenched with water (15 mL) and the mixture was extracted with ethyl acetate (20 mL*3). The organic layer was combined, washed with brine (30 mL), dried over sodium sulfate, filtered and concentrated. The residue was subjected to flash chromatography on silica gel (petroleum ether:ethyl acetate=75:25) to obtain 1-(9-ethyl-6-morpholino-9H-purin-2-yl)-4-phenyl-1H-pyrazol-5-ol (90 mg, 35%) as a yellow solid. LCMS (ESI) m/z: 392.1 [M+H]⁺.

### Step 4: Synthesis of 4-(2-(5-chloro-4-phenyl-1H-pyrazol-1-yl)-9-ethyl-9H-purin-6-yl)morpholine

A solution of 1-(9-ethyl-6-morpholino-9H-purin-2-yl)-4-phenyl-1H-pyrazol-5-ol (90 mg, 0.230 mmol) in phosphoryl trichloride (10 mL) was stirred at 100° C. under nitrogen for 3 h. It was then concentrated and residue was subjected to by prep-HPLC (0.05% ammonium bicarbonate:acetonitrile=5%-95%) to obtain 4-(2-(3-chloro-4-phenyl-1H-pyrazol-1-yl)-9-ethyl-9H-purin-6-yl)morpholine (22.3 mg, 23.7%) as a white solid. ¹H NMR (400 MHz, DMSO-d₆) δ 8.36 (s, 1H), 8.15 (s, 1H), 7.70 (dd, J=8.2, 1.0 Hz, 2H), 7.49 (t, J=7.7 Hz, 2H), 7.38 (t, J=7.4 Hz, 1H), 4.60-4.05 (m, 6H), 3.83-3.66 (m, 4H), 1.43 (t, J=7.3 Hz, 3H). LCMS (ESI) m/z: 409.8 [M+H]⁺.

### Synthesis of 4-(9-ethyl-6-(4-hydroxypiperidin-1-yl)-2-(3-methoxy-4-phenyl-1H-pyrazol-1-yl)-9H-purin-8-yl)-1-methylpiperazin-2-one (Compound 437)

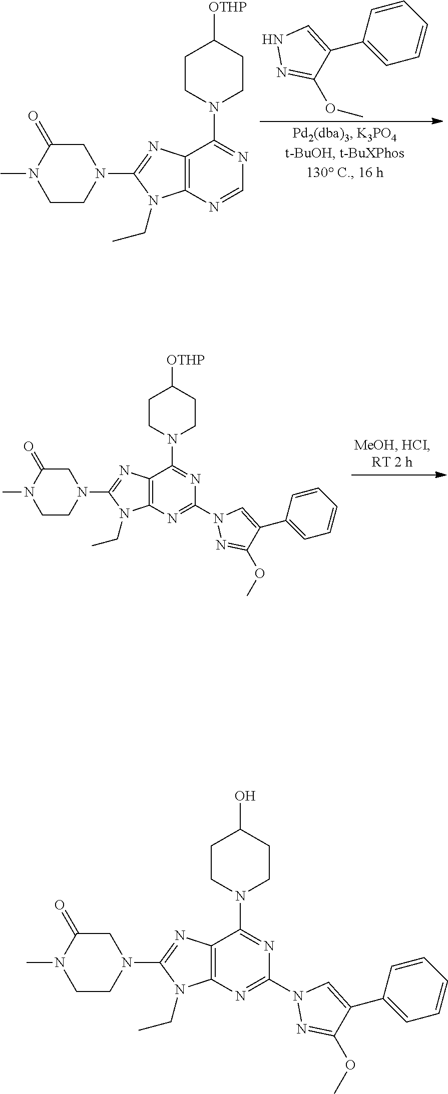

### Step 1: Synthesis of 4-(9-ethyl-2-(3-methoxy-4-phenyl-1H-pyrazol-1-yl)-6-(4-((tetrahydro-2H-pyran-2-yl)oxy)piperidin-1-yl)-9H-purin-8-yl)-1-methylpiperazin-2-one

To a solution of 4-(2-chloro-9-ethyl-6-(4-((tetrahydro-2H-pyran-2-yl)oxy)piperidin-1-yl)-9H-purin-8-yl)-1-methylpiperazin-2-one (150 mg, 0.31 mmol) in tert-butanol (10 mL) were added 3-methoxy-4-phenyl-1H-pyrazole (60.1 mg, 0.35 mmol), tris(dibenzylidene acetone)dipalladium(0) (28.7 mg, 0.03 mmol), potassium phosphate (199.8 mg, 0.94 mmol) and 2-di-tert-butylphosphino-2′,4′,6′-trisopropylbinphenyl (13.3 mg, 0.03 mmol) at 25° C. and the reaction mixture was stirred at 130° C. for 16 h under nitrogen protection. The mixture was then extracted with ethyl acetate (20 mL*2), the organic layer was washed with water (10 mL*2), dried over sodium sulfate, and concentrated. The residue was subjected to silica gel column chromatography (3% methanol in dichloromethane) to obtain 4-(9-ethyl-2-(3-methoxy-4-phenyl-1H-pyrazol-1-yl)-6-(4-((tetrahydro-2H-pyran-2-yl)oxy)piperidin-1-yl)-9H-purin-8-yl)-1-methylpiperazin-2-one as white solid (150 mg, 77.6%). LCMS (ESI) m/z: 616.5 [M+H]⁺.

### Step 2: Synthesis of 4-(9-ethyl-6-(4-hydroxypiperidin-1-yl)-2-(3-methoxy-4-phenyl-1H-pyrazol-1-yl)-9H-purin-8-yl)-1-methylpiperazin-2-one

To a solution of 4 4-(9-ethyl-2-(3-methoxy-4-phenyl-1H-pyrazol-1-yl)-6-(4-((tetrahydro-2H-pyran-2-yl)oxy)piperidin-1-yl)-9H-purin-8-yl)-1-methylpiperazin-2-one (100 mg, 0.16 mmol) in methanol (2 mL) was added methanolic hydrochloric acid solution (2 mL) and the reaction mixture was stirred at 25° C. for 2 h under nitrogen protection. The mixture was then concentrated to obtain 4-(9-ethyl-6-(4-hydroxypiperidin-1-yl)-2-(3-methoxy-4-phenyl-1H-pyrazol-1-yl)-9H-purin-8-yl)-1-methylpiperazin-2-one as white solid (49.0 mg, 56.8%). ¹H NMR (400 MHz, DMSO-d₆) δ 8.85 (s, 1H), 7.80 (d, J=7.2 Hz, 2H), 7.39 (t, J=7.7 Hz, 2H), 7.24 (t, J=7.4 Hz, 1H), 4.82 (s, 3H), 4.14 (q, J=7.1 Hz, 2H), 4.06 (s, 3H), 3.86 (s, 2H), 3.83-3.75 (m, 1H), 3.62 (s, 2H), 3.50 (m, 4H), 2.91 (s, 3H), 1.88 (d, J=9.1 Hz, 2H), 1.41 (m, 5H). LCMS (ESI) m/z: 532.3 [M+H]⁺.

### Synthesis of (S)-1-(9-ethyl-2-(3-methoxy-4-phenyl-1H-pyrazol-1-yl)-6-morpholino-9H-purin-8-yl)pyrrolidin-3-ol (Compound 438)

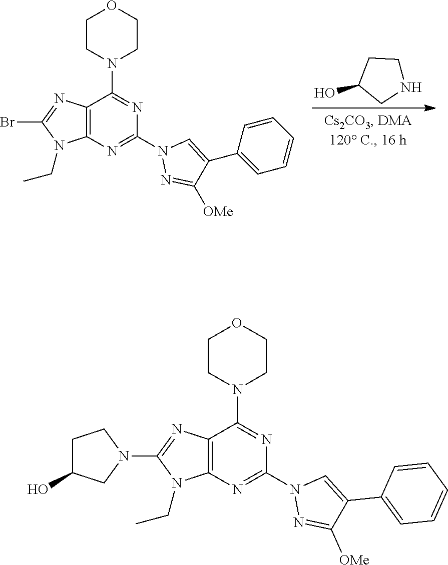 [SMILES: CCn1c(Br)nc2c(N3CCOCC3)nc(-n3cc(-c4ccccc4)c(OC)n3)nc21 ; O[C@H]1CCNC1 ; CCn1c(N2CC[C@H](O)C2)nc2c(N3CCOCC3)nc(-n3cc(-c4ccccc4)c(OC)n3)nc21]

A mixture of 4-(8-bromo-9-ethyl-2-(3-methoxy-4-phenyl-1H-pyrazol-1-yl)-9H-purin-6-yl)morpholine (96.8 mg, 0.2 mmol), (S)-pyrrolidin-3-ol (17.4 mg, 0.2 mmol) and cesium carbonate (163 mg, 0.5 mmol) in N,N-dimethylaniline (4 mL) was stirred at 120° C. under argon atmosphere for 16 h. The reaction mixture was filtered and the filtrate was purified by prep-HPLC (Column Xbridge 21.2*250 mm C18, 10 um, Mobile Phase A: water (10 mmol/L ammonium bicarbonate) B: acetonitrile) to obtain (S)-1-(9-ethyl-2-(3-methoxy-4-phenyl-1H-pyrazol-1-yl)-6-morpholino-9H-purin-8-yl)pyrrolidin-3-ol (7.5 mg, 8%) as a white solid. ¹H NMR (400 MHz, DMSO-d₆) δ 8.87 (d, J=11.5 Hz, 1H), 7.80 (d, J=7.3 Hz, 2H), 7.39 (t, J=7.8 Hz, 2H), 7.23 (t, J=7.4 Hz, 1H), 5.02 (d, J=3.6 Hz, 1H), 4.39 (s, 1H), 4.29-4.15 (m, 6H), 4.06 (s, 3H), 3.72 (d, J=13.7 Hz, 6H), 3.57 (s, 1H), 3.40 (s, 1H), 2.00 (s, 1H), 1.88 (s, 1H), 1.31 (t, J=7.1 Hz, 3H). LCMS (ESI) m/z: 491.3 [M+H]+.

### Synthesis of 4-(2-(3-methoxy-4-phenyl-1H-pyrazol-1-yl)-8-methyl-9-((methylsulfinyl)methyl)-9H-purin-6-yl)morpholine (Compound 439)

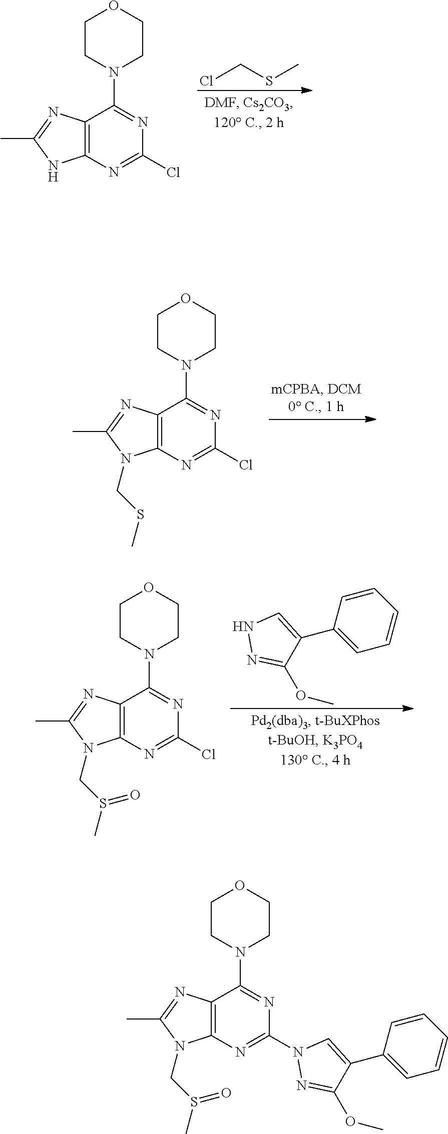

### Step 1: Synthesis of 4-(2-chloro-8-methyl-9-((methylthio)methyl)-9H-purin-6-yl)morpholine

To a solution of 4-(2-chloro-8-methyl-9H-purin-6-yl)morpholine (300 mg, 1.18 mmol) in N,N-dimethylformamide (5 mL) were added (chloromethyl)(methyl)sulfane (161.4 mg, 1.67 mmol) and cesium carbonate (816.7 mg, 2.51 mmol) and the reaction mixture was stirred at 120° C. for 2 h under nitrogen protection. The mixture was then extracted with ethyl acetate (20 mL*2), the organic layer was washed with water (10 mL*2), dried over sodium sulfate, and concentrated. The residue was subjected to silica gel column chromatography (1% methanol in dichloromethane) to obtain 4-(2-chloro-8-methyl-9-((methylthio)methyl)-9H-purin-6-yl)morpholine as white solid (300.0 mg, 80.9%). LCMS (ESI) m/z: 314.2 [M+H]⁺.

### Step 2: Synthesis of 2-(2-chloro-8-methyl-6-morpholino-9H-purin-9-yl)ethan-1-ol

To a solution of 4-(2-chloro-8-methyl-9-((methylthio)methyl)-9H-purin-6-yl)morpholine (250.0 mg, 0.80 mmol) in dichloromethane (20 mL) was added 3-chloroperoxybenzoic acid (137.5 mg, 0.79 mmol) at 0° C. and the reaction mixture was stirred at 25° C. for 2 h under nitrogen protection. The mixture was extracted with ethyl acetate (20 mL*2), the organic layer was washed with water (10 mL*2), dried over sodium sulfate, and concentrated. The residue was subjected to silica gel column chromatography (3% methanol in dichloromethane) to obtain 4-(2-chloro-8-methyl-9-((methylsulfinyl)methyl)-9H-purin-6-yl)morpholine as yellow solid (200.0 mg, 76.3%). LCMS (ESI) m/z: 330.1 [M+H]⁺.

### Step 3: Synthesis of 4-(2-(3-methoxy-4-phenyl-1H-pyrazol-1-yl)-8-methyl-9-((methylsulfinyl)methyl)-9H-purin-6-yl)morpholine

To a solution of 4-(2-chloro-8-methyl-9-((methylsulfinyl)methyl)-9H-purin-6-yl)morpholine (150 mg, 0.45 mmol) in tert-butanol (9 mL) were added 3-methoxy-4-phenyl-1H-pyrazole (87.2 mg, 0.50 mmol), tris(dibenzylideneacetone)dipalladium(0) (41.6 mg, 0.04 mmol), potassium phosphate (289.6 mg, 1.36 mmol) and 2-di-tert-butylphosphino-2′,4′,6′-trisopropylbinphenyl (25.6 mg, 0.04 mmol) at 25° C. and the reaction mixture was stirred at 130° C. for 16 h under nitrogen protection. The mixture was extracted with ethyl acetate (20 mL*2), the organic layer was washed with water (10 mL*2), dried over sodium sulfate, and concentrated. The residue was subjected to silica gel column chromatography (5% methanol in dichloromethane) to obtain 4-(2-(3-methoxy-4-phenyl-1H-pyrazol-1-yl)-8-methyl-9-((methylsulfinyl)methyl)-9H-purin-6-yl)morpholine as white solid (22.5 mg, 10.6%). ¹H NMR (400 MHz, DMSO-d₆) δ 8.94 (s, 1H), 7.79 (d, J=7.3 Hz, 2H), 7.40 (t, J=7.7 Hz, 2H), 7.25 (t, J=7.4 Hz, 1H), 5.46 (m, 2H), 4.28 (s, 4H), 4.06 (s, 3H), 3.82-3.72 (m, 4H), 2.71 (s, 3H), 2.58 (s, 3H). LCMS (ESI) m/z: 468.3 [M+H]⁺.

### Synthesis of 1-(2-(3-methoxy-4-phenyl-1H-pyrazol-1-yl)-8-methyl-6-morpholino-9H-purin-9-yl)-2-methylpropan-2-ol (Compound 440):)

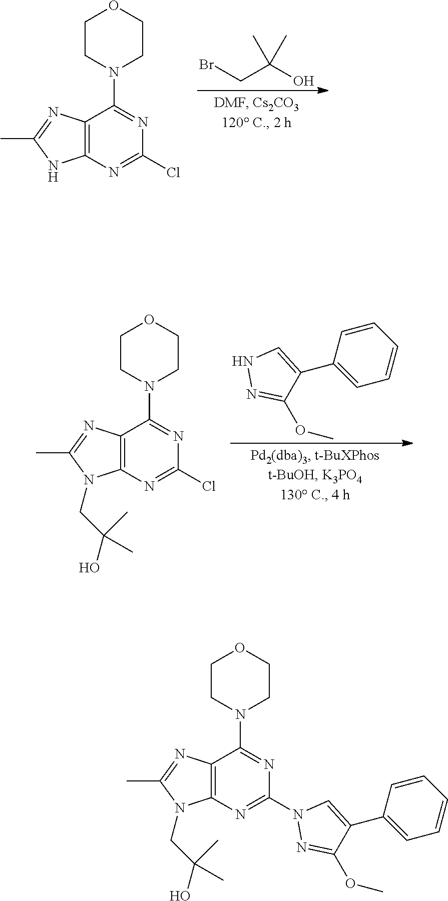

### Step 1: Synthesis of 1-(2-chloro-8-methyl-6-morpholino-9H-purin-9-yl)-2-methylpropan-2-ol

To a solution of 4-(2-chloro-8-methyl-9H-purin-6-yl)morpholine (300 mg, 1.18 mmol) in N,N-dimethylformamide (5 mL) were added 1-bromo-2-methylpropan-2-ol (217.14 mg, 1.42 mmol) and cesium carbonate (1.15 g, 3.54 mmol) and the reaction mixture was stirred at 120° C. for 2 h under nitrogen atmosphere. The mixture was then extracted with ethyl acetate (20 mL*2), the organic layer was washed with water (10 mL*2), dried over sodium sulfate, and concentrated. The residue was subjected to silica gel column chromatography (1% methanol in dichloromethane) to obtain 1-(2-chloro-8-methyl-6-morpholino-9H-purin-9-yl)-2-methylpropan-2-ol as white solid (200.0 mg, 51.9%). LCMS (ESI) m/z: 326.3 [M+H]⁺.

### Step 2: Synthesis of 1-(2-(3-methoxy-4-phenyl-1H-pyrazol-1-yl)-8-methyl-6-morpholino-9H-purin-9-yl)-2-methylpropan-2-ol

To a solution of 1-(2-chloro-8-methyl-6-morpholino-9H-purin-9-yl)-2-methylpropan-2-ol (150 mg, 0.46 mmol) in tert-butanol (10 mL) was added 3-methoxy-4-phenyl-1H-pyrazole (87.2 mg, 0.50 mmol), tris(dibenzylideneacetone)dipalladium(0) (42.2 mg, 0.04 mmol), potassium phosphate (293.2 mg, 1.38 mmol) and 2-di-tert-butylphosphino-2′,4′,6′-trisopropylbinphenyl (25.6 mg, 0.04 mmol) at 25° C. and the reaction mixture was stirred at 130° C. for 16 h under nitrogen atmosphere. The resultant mixture was extracted with ethyl acetate (20 mL*2), the combined organic layer was washed with water (10 mL*2), dried over sodium sulfate, and concentrated. The residue was subjected to silica gel column chromatography (5% methanol in dichloromethane) to afford 1-(2-(3-methoxy-4-phenyl-1H-pyrazol-1-yl)-8-methyl-6-morpholino-9H-purin-9-yl)-2-methylpropan-2-ol as white solid (23.1 mg, 10.8%). ¹H NMR (400 MHz, DMSO-d₆) δ 8.88 (s, 1H), 7.79 (d, J=7.3 Hz, 2H), 7.40 (t, J=7.7 Hz, 2H), 7.24 (t, J=7.4 Hz, 1H), 4.92 (bs, 1H), 4.28 (bs, 4H), 4.09 (s, 2H), 4.05 (s, 3H), 3.84-3.68 (m, 4H), 2.56 (s, 3H), 1.18 (s, 6H). LCMS (ESI) m/z: 464.4 [M+H]⁺.

### Synthesis of 4-(9-(cyclopropylmethyl)-2-(3-methoxy-4-phenyl-1H-pyrazol-1-yl)-9H-purin-6-yl)morpholine (Compound 441)

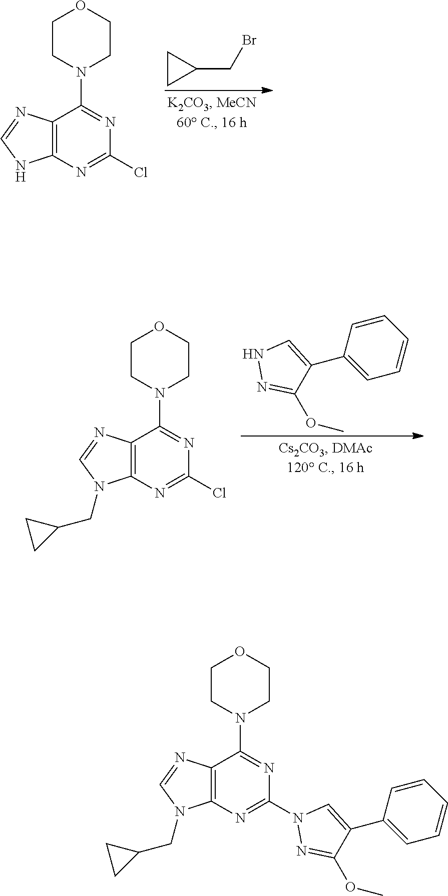 [SMILES: Clc1nc(N2CCOCC2)c2nc[nH]c2n1 ; BrCC1CC1 ; Clc1nc(N2CCOCC2)c2ncn(CC3CC3)c2n1 ; COc1n[nH]cc1-c1ccccc1 ; COc1nn(-c2nc(N3CCOCC3)c3ncn(CC4CC4)c3n2)cc1-c1ccccc1]

### Step 1: Synthesis of 4-(2-chloro-9-(cyclopropylmethyl)-9H-purin-6-yl)morpholine

A mixture of 4-(2-chloro-9H-purin-6-yl)morpholine (720 mg, 3 mmol), (bromomethyl)cyclopropane (405 mg, 3 mmol), potassium carbonate (1242 mg, 9 mmol) and acetonitrile (10 mL) was stirred at 60° C. for 16 h. The resultant precipitates were collected and dried to afford 4-(2-chloro-9-(cyclopropylmethyl)-9H-purin-6-yl)morpholine (850 mg, crude) as white solid. LCMS (ESI) m/z: 294.3 [M+H]⁺.

### Step 2: Synthesis of 4-(9-(cyclopropylmethyl)-2-(3-methoxy-4-phenyl-1H-pyrazol-1-yl)-9H-purin-6-yl)morpholine

A mixture of 4-(2-chloro-9-(cyclopropylmethyl)-9H-purin-6-yl)morpholine (420 mg, 1.4 mmol), 3-methoxy-4-phenyl-1H-pyrazole (248 mg, 1.4 mmol) and cesium carbonate (1164 mg, 3.6 mmol) in DMAc (20 mL) was stirred at 120° C. under argon atmosphere for 16 h. The reaction mixture was filtered, the filtrate was subjected to prep-HPLC (Column Xbridge 21.2*250 mm C18, 10 um, Mobile Phase A: water (10 mmol/L ammonium bicarbonate) B: acetonitrile) to obtain 4-(9-(cyclopropylmethyl)-2-(3-methoxy-4-phenyl-1H-pyrazol-1-yl)-9H-purin-6-yl)morpholine (31.3 mg, 5%) as white solid. ¹H NMR (400 MHz, CDCl₃) δ 7.68 (s, 1H), 7.55 (s, 1H), 7.51-7.45 (m, 2H), 7.23 (d, J=7.8 Hz, 2H), 7.12 (t, J=7.4 Hz, 1H), 4.51-3.99 (m, 4H), 3.90 (s, 2H), 3.89 (s, 3H), 3.67 (s, 4H), 1.23-1.15 (m, 1H), 0.56 (q, J=5.9 Hz, 2H), 0.36 (q, J=4.8 Hz, 2H). LCMS (ESI) m/z: 432.3 [M+H]⁺.

### Synthesis of 4-(2-(3-chloro-4-phenyl-1H-pyrazol-1-yl)-9-ethyl-9H-purin-6-yl)morpholine (Compound 442)

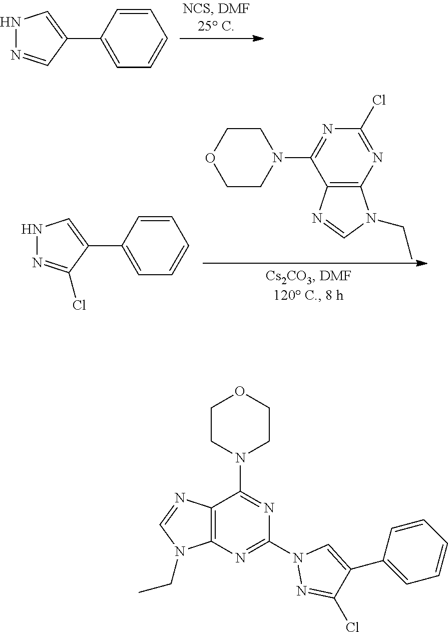 [SMILES: c1ccc(-c2cn[nH]c2)cc1 ; Clc1n[nH]cc1-c1ccccc1 ; CCn1cnc2c(N3CCOCC3)nc(Cl)nc21 ; CCn1cnc2c(N3CCOCC3)nc(-n3cc(-c4ccccc4)c(Cl)n3)nc21]

### Step 1: Synthesis of 3-chloro-4-phenyl-1H-pyrazole

To a solution of 4-phenyl-1H-pyrazole (210 mg, 1.458 mmol) in DMF (10 mL) was added 1-chloropyrrolidine-2,5-dione (193 mg, 1.458 mmol). The resultant mixture was stirred at 30° C. for 8 h, then quenched with water (15 mL) and extracted with ethyl acetate (20 mL*3). The organic layers were combined, washed with brine (30 mL), dried over sodium sulfate, filtered and concentrated. The residue was purified by flash chromatography on silica gel (petroleum ether:ethyl acetate=75:25) to give 3-chloro-4-phenyl-1H-pyrazole as yellow solid. (100 mg, 38.5%). LCMS (ESI) m/z: 179.1[M+H]⁺.

### Step 2: Synthesis of 4-(2-(3-chloro-4-phenyl-1H-pyrazol-1-yl)-9-ethyl-9H-purin-6-yl)morpholine

To a solution of 3-chloro-4-phenyl-1H-pyrazole (100 mg, 0.526 mmol) in DMF (5 mL) were added 4-(2-chloro-9-ethyl-9H-purin-6-yl)morpholine (140 mg, 0.526 mmol) and cesium carbonate (342 mg, 1.05 mmol). The resultant mixture was stirred at 120° C. for 8 h, then quenched with water (15 mL) and extracted with ethyl acetate (20 mL*3). The organic layers were combined, washed with brine (30 mL), dried over sodium sulfate, filtered and concentrated. The residue was subjected to prep-HPLC (0.05% ammonium bicarbonate:acetonitrile=5%-95%) to obtain 4-(2-(3-chloro-4-phenyl-1H-pyrazol-1-yl)-9-ethyl-9H-purin-6-yl)morpholine (117.7 mg, 54.7%) as a white solid. ¹H NMR (400 MHz, DMSO-d₆) δ 9.03 (s, 1H), 8.27 (s, 1H), 7.78-7.70 (m, 2H), 7.49 (t, J=7.6 Hz, 2H), 7.39 (dd, J=7.1, 1.2 Hz, 1H), 4.45-4.05 (m, 6H), 3.81-3.72 (m, 4H), 1.44 (t, J=7.3 Hz, 3H). LCMS (ESI) m/z: 409.8[M+H]+.

### Synthesis of 4-(9-ethyl-6-(4-hydroxypiperidin-1-yl)-2-(3-(1-methyl-1H-pyrazol-3-yl)phenyl)-9H-purin-8-yl)-1-methylpiperazin-2-one (Compound 443)

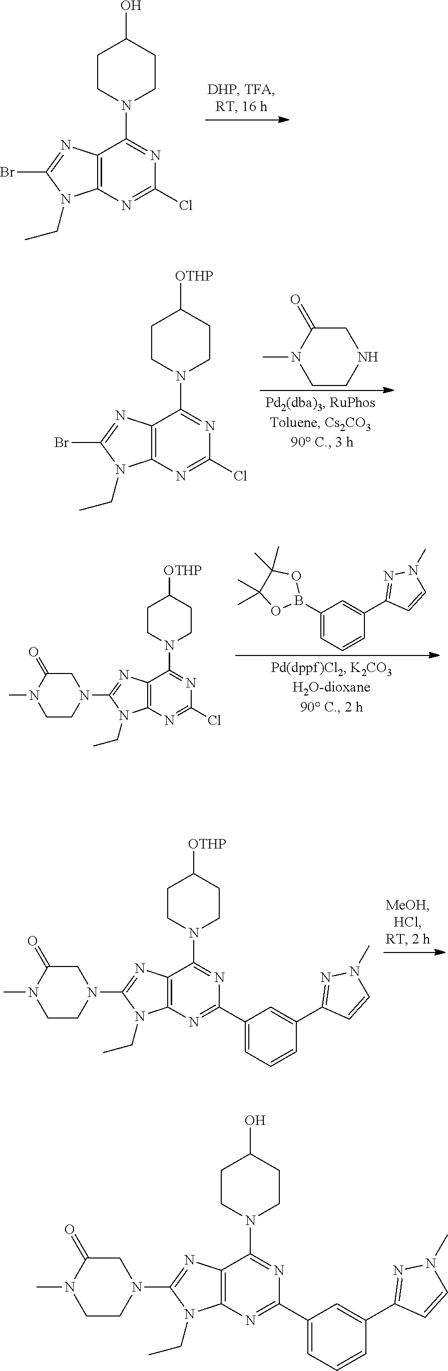

### Step 1: Synthesis of 8-bromo-2-chloro-9-ethyl-6-(4-((tetrahydro-2H-pyran-2-yl)oxy)piperidin-1-yl)-9H-purine

To a solution of 1-(8-bromo-2-chloro-9-ethyl-9H-purin-6-yl)piperidin-4-ol (1 g, 2.78 mmol) in dichloromethane (50 mL) were added 3,4-dihydro-2H-pyran (328.0 mg, 3.89 mmol) and trifluoroacetic acid (5 drops, 0.27 mmol). The resultant mixture was stirred at 25° C. for 16 h under nitrogen protection. The mixture was then extracted with ethyl acetate (20 mL*2), the organics were washed with water (10 mL*2), dried over sodium sulfate and concentrated. The residue was subjected to silica gel column chromatography (15% ethyl acetate in petroleum ether) to obtain 8-bromo-2-chloro-9-ethyl-6-(4-((tetrahydro-2H-pyran-2-yl)oxy)piperidin-1-yl)-9H-purine as white solid (600.0 mg, 48.8%). LCMS (ESI) m/z: 444.1 [M+H]⁺.

### Step 2: Synthesis of 4-(2-chloro-9-ethyl-6-(4-((tetrahydro-2H-pyran-2-yl)oxy)piperidin-1-yl)-9H-purin-8-yl)-1-methylpiperazin-2-one

To a solution of 8-bromo-2-chloro-9-ethyl-6-(4-((tetrahydro-2H-pyran-2-yl)oxy)piperidin-1-yl)-9H-purine (500 mg, 1.13 mmol) in toluene (15 mL) were added 1-methylpiperazin-2-one (64.4 mg, 0.56 mmol), tris(dibenzylideneacetone)dipalladium(0) (103.3 mg, 0.11 mmol), 2-dicyclohexylphosphino-2′,6′-di-i-propoxy-1,1′-biphenyl (52.7 mg, 0.11 mmol) and cesium carbonate (1.10 g, 3.38 mmol). The resultant reaction mixture was stirred at 90° C. for 3 h under nitrogen atmosphere. It was then extracted with ethyl acetate (20 mL*2), the combined organic layer was washed with water (10 mL*2), dried over sodium sulfate and concentrated. The residue was subjected to silica gel column chromatography (15% ethyl acetate in petroleum ether) to obtain 4-(2-chloro-9-ethyl-6-(4-((tetrahydro-2H-pyran-2-yl)oxy)piperidin-1-yl)-9H-purin-8-yl)-1-methylpiperazin-2-one as white solid (450.0 mg, 83.6%). LCMS (ESI) m/z: 478.3 [M+H]⁺.

### Step 3: Synthesis of 4-(9-ethyl-2-(3-(1-methyl-1H-pyrazol-3-yl)phenyl)-6-(4-((tetrahydro-2H-pyran-2-yl)oxy)piperidin-1-yl)-9H-purin-8-yl)-1-methylpiperazin-2-one

To a solution of 4-(2-chloro-9-ethyl-6-(4-((tetrahydro-2H-pyran-2-yl)oxy)piperidin-1-yl)-9H-purin-8-yl)-1-methylpiperazin-2-one (200 mg, 0.42 mmol) in 1,4-dioxane (6 mL) and water (2 mL) were added 1-methyl-3-(3-(4,4,5,5-tetramethyl-1,3,2-dioxaborolan-2-yl)phenyl)-1H-pyrazole (142.7 mg, 0.50 mmol), [1,1′-bis(diphenylphosphino)ferrocene]dichloropalladium(II) (30.62 mg, 0.04 mmol) and potassium carbonate (174.1 mg, 1.25 mmol). The resultant mixture was stirred at 90° C. for 2 h under nitrogen atmosphere. It was then extracted with ethyl acetate (20 mL*2), the combined organic layer was washed with water (10 mL*2), dried over sodium sulfate, and concentrated. The residue was subjected to silica gel column chromatography (3% methanol in dichloromethane) to obtain 4-(9-ethyl-2-(3-(1-methyl-1H-pyrazol-3-yl)phenyl)-6-(4-((tetrahydro-2H-pyran-2-yl)oxy)piperidin-1-yl)-9H-purin-8-yl)-1-methylpiperazin-2-one as white solid (150.0 mg, 60.0%). LCMS (ESI) m/z: 600.4 [M+H]⁺.

### Step 4: Synthesis of 4-(9-ethyl-6-(4-hydroxypiperidin-1-yl)-2-(3-(1-methyl-1H-pyrazol-3-yl)phenyl)-9H-purin-8-yl)-1-methylpiperazin-2-one

To a solution of 4-(9-ethyl-2-(3-(1-methyl-1H-pyrazol-3-yl)phenyl)-6-(4-((tetrahydro-2H-pyran-2-yl)oxy)piperidin-1-yl)-9H-purin-8-yl)-1-methylpiperazin-2-one (100 mg, 0.16 mmol) in methanol (2 mL) was added methanolic hydrochloric acid solution (2 mL) and the reaction mixture was stirred at 25° C. for 2 h under nitrogen atmosphere. The reaction mixture was then concentrated to obtain 4-(9-ethyl-6-(4-hydroxypiperidin-1-yl)-2-(3-(1-methyl-1H-pyrazol-3-yl)phenyl)-9H-purin-8-yl)-1-methylpiperazin-2-one as white solid (72.5 mg, 84.3%). ¹H NMR (400 MHz, DMSO-d₆) δ 8.73 (s, 1H), 8.27 (d, J=7.8 Hz, 1H), 7.80 (m, 2H), 7.48 (t, J=7.7 Hz, 1H), 6.72 (d, J=2.2 Hz, 1H), 4.87 (d, J=12.6 Hz, 3H), 4.20 (q, J=7.0 Hz, 2H), 3.92 (s, 3H), 3.89 (s, 2H), 3.84-3.73 (m, 1H), 3.66-3.52 (m, 4H), 3.49 (d, J=5.3 Hz, 2H), 2.91 (s, 3H), 1.88 (d, J=9.3 Hz, 2H), 1.44 (t, J=7.1 Hz, 5H). LCMS (ESI) m/z: 516.4 [M+H]⁺.

### Synthesis of (R)-1-(9-ethyl-2-(3-methoxy-4-phenyl-1H-pyrazol-1-yl)-6-morpholino-9H-purin-8-yl)pyrrolidin-3-ol (Compound 444)

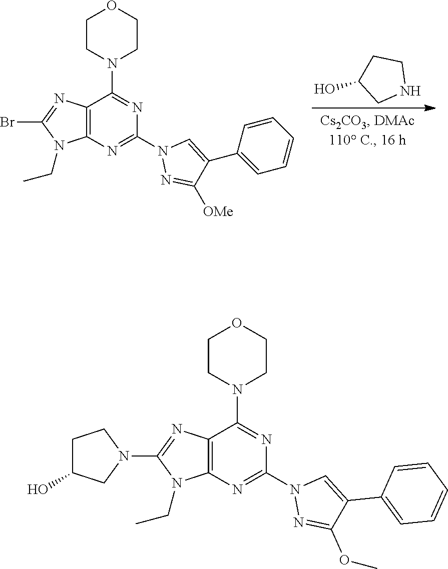 [SMILES: CCn1c(Br)nc2c(N3CCOCC3)nc(-n3cc(-c4ccccc4)c(OC)n3)nc21 ; O[C@@H]1CCNC1 ; CCn1c(N2CC[C@@H](O)C2)nc2c(N3CCOCC3)nc(-n3cc(-c4ccccc4)c(OC)n3)nc21]

A solution of 4-(8-bromo-9-ethyl-2-(3-methoxy-4-phenyl-1H-pyrazol-1-yl)-9H-purin-6-yl)morpholine (40 mg, 0.083 mmol), (R)-pyrrolidin-3-ol (9 mg, 0.1 mmol) and cesium carbonate (82 mg, 0.25 mmol) in dry DMAc (10 mL) was stirred at 110° C. for 16 h. The resultant reaction mixture was filtered to remove the solids and the filtrate was concentrated and subjected to prep-HPLC (BOSTON pHlex ODS 10 μm 21.2×250 mm 120 A. The mobile phase was acetonitrile/0.1% ammonium bicarbonate) to obtain (R)-1-(9-ethyl-2-(3-methoxy-4-phenyl-1H-pyrazol-1-yl)-6-morpholino-9H-purin-8-yl)pyrrolidin-3-ol (25.5 mg, 42.67%) as white solid. ¹H NMR (400 MHz, DMSO-d₆) δ 8.86 (s, 1H), 7.80 (d, J=7.3 Hz, 2H), 7.39 (t, J=7.7 Hz, 2H), 7.23 (t, J=7.4 Hz, 1H), 5.02 (d, J=3.6 Hz, 1H), 4.39 (s, 1H), 4.29-4.12 (m, 6H), 4.06 (s, 3H), 3.73 (dd, J=11.0, 6.9 Hz, 6H), 3.58 (d, J=8.6 Hz, 1H), 3.39 (d, J=10.9 Hz, 1H), 2.04-1.98 (m, 1H), 1.88 (s, 1H), 1.31 (t, J=7.1 Hz, 3H). LCMS (ESI) m/z: 491.3 [M+H]⁺.

### Synthesis of 1-(9-ethyl-2-(3-methoxy-4-phenyl-1H-pyrazol-1-yl)-6-morpholino-9H-purin-8-yl)azetidin-3-ol (Compound 445)

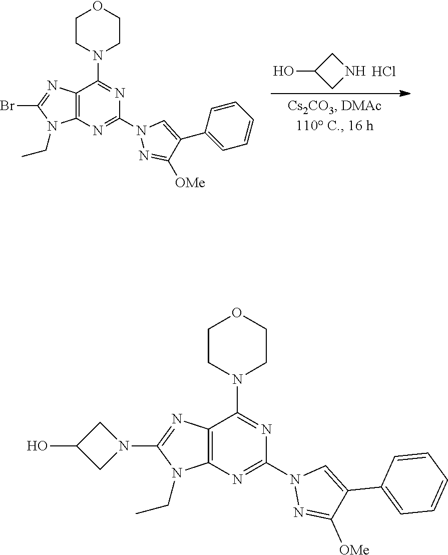 [SMILES: CCn1c(Br)nc2c(N3CCOCC3)nc(-n3cc(-c4ccccc4)c(OC)n3)nc21 ; OC1CNC1 ; Cl ; CCn1c(N2CC(O)C2)nc2c(N3CCOCC3)nc(-n3cc(-c4ccccc4)c(OC)n3)nc21]

A mixture of 4-(8-bromo-9-ethyl-2-(3-methoxy-4-phenyl-1H-pyrazol-1-yl)-9H-purin-6-yl)morpholine (30 mg, 0.06 mmol), azetidin-3-ol hydrochloride (7 mg, 0.07 mmol) and cesium carbonate (60 mg, 0.18 mmol) in N,N-dimethylacetamide (2 mL) was stirred at 110° C. under argon atmosphere for 16 h. Then water was added and the mixture was extracted with ethyl acetate (50 mL×3). The combined organic layer was dried and concentrated. The residue was subjected to prep-HPLC to obtain 1-(9-ethyl-2-(3-methoxy-4-phenyl-1H-pyrazol-1-yl)-6-morpholino-9H-purin-8-yl)azetidin-3-ol (20 mg, 70%) as white solid. ¹H NMR (400 MHz, DMSO-d₆) δ 8.86 (s, 1H), 7.80 (d, J=7.2 Hz, 2H), 7.39 (t, J=7.7 Hz, 2H), 7.23 (t, J=7.4 Hz, 1H), 5.74 (d, J=6.6 Hz, 1H), 4.64-4.54 (m, 1H), 4.32 (q, J=7.2 Hz, 2H), 4.20 (s, 4H), 4.07-4.00 (m, 5H), 3.92 (dd, J=8.3, 5.3 Hz, 2H), 3.77-3.72 (m, 4H), 1.31 (t, J=7.2 Hz, 3H). LCMS (ESI) m/z: 477.1 [M+H]⁺.

### Synthesis of 2-(9-ethyl-2-(3-(1-methyl-1H-pyrazol-3-yl)phenyl)-6-morpholino-9H-purin-8-yl)ethan-1-ol (Compound 446)

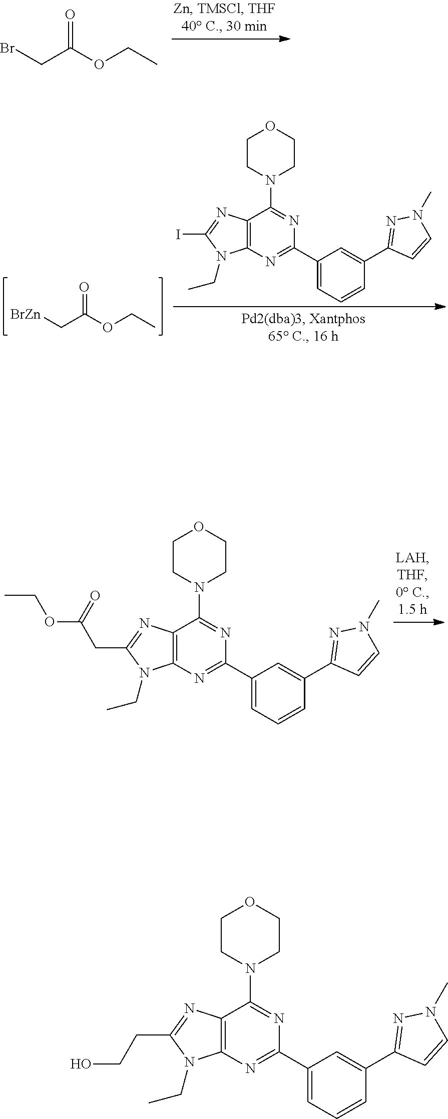

### Step 1: Synthesis of ethyl 2-(9-ethyl-2-(3-(1-methyl-1H-pyrazol-3-yl)phenyl)-6-morpholino-9H-purin-8-yl)acetate

To a suspension of zinc powder (2.56 g, 39.15 mmol) in tetrahydrofuran (20 mL) was added trimethylsilyl chloride (0.25 mL, 2.9 mmol) at room temperature under nitrogen atmosphere and the resultant mixture was stirred for 30 min. The reaction mixture was then heated to 40° C. followed by the drop-wise addition of ethyl 2-bromoacetate (2.2 mL, 19.65 mmol) and the stirring was continued at 40° C. for 30 min. After the insoluble matter precipitated, the light-yellow supernatant solution was decanted and used for subsequent experiments.

To a stirred solution of 4-(9-ethyl-8-iodo-2-(3-(1-methyl-1H-pyrazol-3-yl)phenyl)-9H-purin-6-yl)morpholine (0.25 g, 0.48 mmol), tris(dibenzylideneacetone)dipalladium (45 mg, 0.048 mmol) and 4,5-bis(diphenylphosphino)-9,9-dimethylxanthene (56 mg, 0.097 mmol) in tetrahydrofuran (10 mL) was added the above zinc bromide solution (2.5 mL, 2.5 mmol) drop-wise at room temperature under argon atmosphere. After the addition, the reaction mixture was heated to 65° C. and stirred for 16 h. The reaction mixture was cooled down, quenched with aqueous ammonium chloride solution and extracted with ethyl acetate (20 mL×2). The combined organic phase was washed with brine (20 mL), dried over sodium sulfate, filtered and concentrated. The residue was subjected to flash chromatography (eluted with ethyl acetate in petroleum ether from 20% to 40%) to obtain ethyl 2-(9-ethyl-2-(3-(1-methyl-1H-pyrazol-3-yl)phenyl)-6-morpholino-9H-purin-8-yl)acetate (0.1 g, 43.9%) as yellow solid. LCMS (ESI) m/z: 475.8 [M+H]⁺.

### Step 2: Synthesis of 2-(9-ethyl-2-(3-(1-methyl-1H-pyrazol-3-yl)phenyl)-6-morpholino-9H-purin-8-yl)ethan-1-ol

To a solution of ethyl 2-(9-ethyl-2-(3-(1-methyl-1H-pyrazol-3-yl)phenyl)-6-morpholino-9H-purin-8-yl)acetate (80 mg, 0.17 mmol) in tetrahydrofuran (10 mL) at 0° C., was added a solution of 1M lithium aluminum hydride in tetrahydrofuran (0.5 mL, 0.5 mmol) drop-wise under nitrogen atmosphere. After the addition, the reaction mixture was stirred at 0° C. for 1.5 h. The reaction was then quenched with sodium sulfate decahydrate and the solids were filtered off. The filtrate was concentrated and the residue was subjected to prep-HPLC (base) to obtain 2-(9-ethyl-2-(3-(1-methyl-1H-pyrazol-3-yl)phenyl)-6-morpholino-9H-purin-8-yl)ethan-1-ol (30.4 mg, 41.3%) as white solid. ¹H NMR (400 MHz, DMSO-d₆) δ 8.74 (s, 1H), 8.30 (d, J=7.9 Hz, 1H), 7.84 (d, J=7.7 Hz, 1H), 7.77 (t, J=2.2 Hz, 1H), 7.49 (t, J=7.7 Hz, 1H), 6.74 (d, J=2.2 Hz, 1H), 4.86 (t, J=5.4 Hz, 1H), 4.41-4.25 (m, 6H), 3.92 (s, 3H), 3.85 (dd, J=12.1, 6.5 Hz, 2H), 3.81-3.71 (m, 4H), 3.03 (t, J=6.6 Hz, 2H), 1.39 (t, J=7.2 Hz, 3H). LCMS (ESI) m/z: 434.3 [M+H]⁺.

### Synthesis of 4-(2-(3-(difluoromethoxy)-4-phenyl-1H-pyrazol-1-yl)-9-ethyl-9H-purin-6-yl)morpholine (Compound 447)

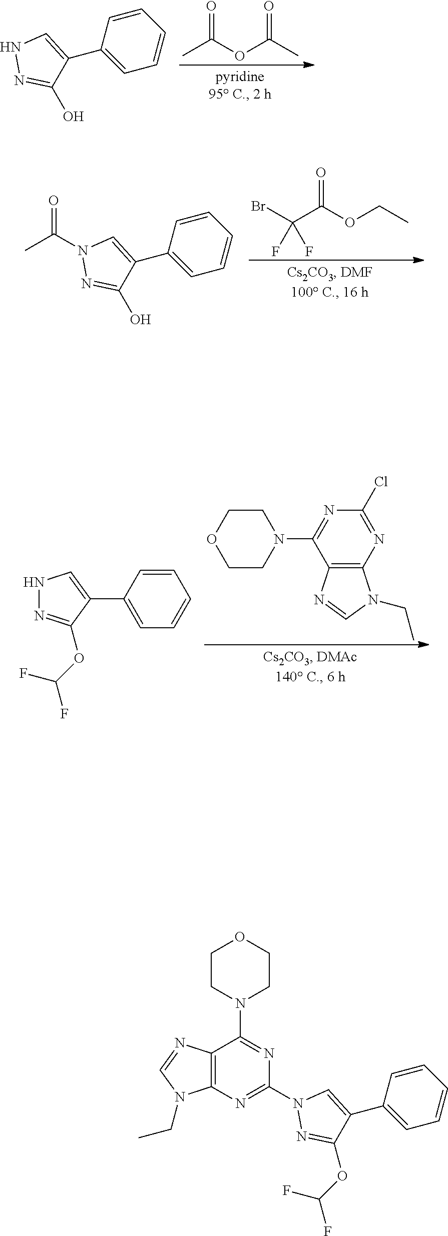

### Step 1: Synthesis of 1-(3-hydroxy-4-phenyl-1H-pyrazol-1-yl)ethenone

A mixture of 4-phenyl-1H-pyrazol-3-ol (160 mg, 1 mmol) in pyridine (0.76 mL, 9.4 mmol) was heated to 95° C. and then charged with a solution of acetic anhydride (0.19 mL, 2 mmol) in pyridine (0.36 mL, 4.4 mmol) over a 5 min period. The reaction mixture was stirred at that temperature for an additional 2 h and concentrated to afford 1-(3-hydroxy-4-phenyl-1H-pyrazol-1-yl)ethanone (200 mg, 99%) as white solid. LCMS (ESI) m/z: 203.1 [M+H]⁺.

### Step 2: Synthesis of 3-(difluoromethoxy)-4-phenyl-1H-pyrazole

A mixture of 1-(3-hydroxy-4-phenyl-1H-pyrazol-1-yl)ethanone (80 mg, 0.4 mmol), ethyl 2-bromo-2,2-difluoroacetate (97 mg, 0.48 mmol) and cesium carbonate (522 mg, 1.6 mmol) in N,N-dimethylformamide (10 mL) was stirred at 100° C. for 16 h. The mixture was then subjected to prep-HPLC (BOSTON pHlex ODS 10 μm 21.2×250 mm 120 A. The mobile phase was acetonitrile/0.1% ammonium bicarbonate) to obtain 3-(difluoromethoxy)-4-phenyl-1H-pyrazole (40 mg, 38%) as white solid. LCMS (ESI) m/z: 210.9 [M+H]⁺.

### Step 3: Synthesis of 4-(2-(3-(difluoromethoxy)-4-phenyl-1H-pyrazol-1-yl)-9-ethyl-9H-purin-6-yl)morpholine

A mixture of 3-(difluoromethoxy)-4-phenyl-1H-pyrazole (20 mg, 0.1 mmol), 4-(2-chloro-9-ethyl-9H-purin-6-yl)morpholine (32 mg, 0.12 mmol) and cesium carbonate (130 mg, 0.4 mmol) in N,N-dimethylacetamide (3 mL) was stirred at 140° C. for 6 h. The mixture was then subjected to prep-HPLC (BOSTON pHlex ODS 10 um 21.2×250 mm 120 A. The mobile phase was acetonitrile/0.1% ammonium bicarbonate) to obtain 4-(2-(3-(difluoromethoxy)-4-phenyl-1H-pyrazol-1-yl)-9-ethyl-9H-purin-6-yl)morpholine (33.2 mg, 38%) as white solid. ¹H NMR (500 MHz, DMSO-d₆) δ 9.05 (s, 1H), 8.26 (s, 1H), 7.80-7.74 (m, 2H), 7.62 (s, 1H), 7.46 (t, J=7.9 Hz, 2H), 7.32 (t, J=7.4 Hz, 1H), 4.35 (bs, 4H), 4.23 (q, J=7.3 Hz, 2H), 3.81-3.73 (m, 4H), 1.45 (t, J=7.3 Hz, 3H). LCMS (ESI) m/z: 442.2[M+H]⁺.

### Synthesis of 2-(2-(3-methoxy-4-phenyl-1H-pyrazol-1-yl)-8-methyl-6-morpholino-9H-purin-9-yl)ethan-1-ol (Compound 448)

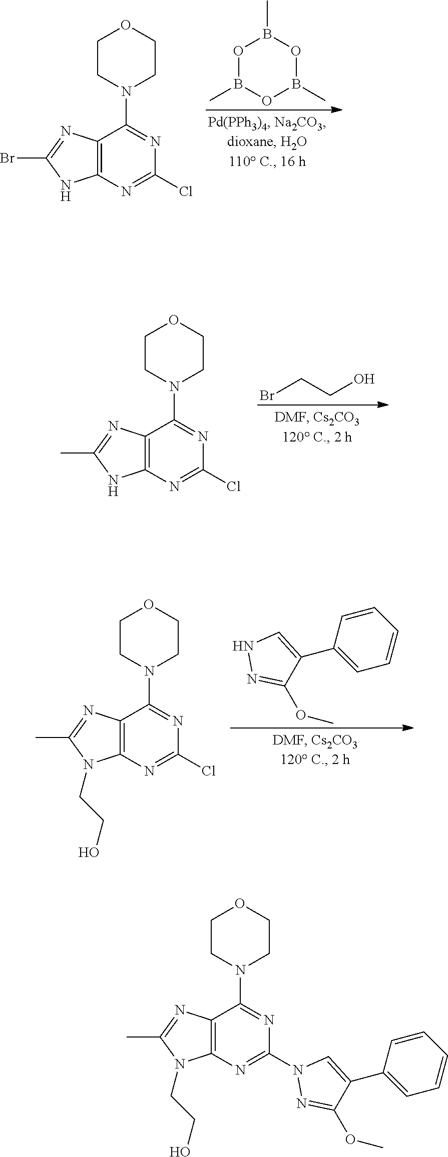

### Step 1: Synthesis of 4-(2-chloro-8-methyl-9H-purin-6-yl)morpholine

To a solution of 4-(8-bromo-2-chloro-9H-purin-6-yl)morpholine (1 g, 3.14 mmol) in 1,4-dioxane (20 mL) and water (4 mL) were added 2,4,6-trimethyl-1,3,5,2,4,6-trioxatriborinane (393.9 mg, 3.14 mmol), tetrakis(triphenylphosphine)palladium (362.7 mg, 0.31 mmol) and sodium carbonate (998.0 mg, 9.42 mmol). The resultant reaction mixture was stirred at 110° C. for 16 h under nitrogen protection. It was then extracted with dichloromethane (20 mL*2) and washed with water (10 mL*2). The organic layer was dried over sodium sulfate, filtered and concentrated. The residue was subjected to silica gel column chromatography (6% methanol in dichloromethane) to obtain 4-(2-chloro-8-methyl-9H-purin-6-yl)morpholine as white solid (600.0 mg, 75.4%). LCMS (ESI) m/z: 254.2 [M+H]⁺.

### Step 2: Synthesis of 2-(2-chloro-8-methyl-6-morpholino-9H-purin-9-yl)ethan-1-ol

To a solution of 4-(2-chloro-8-methyl-9H-purin-6-yl)morpholine (200 mg, 0.78 mmol) in N,N-dimethylformamide (5 mL) were added 2-bromoethan-1-ol (139.2 mg, 1.58 mmol) and cesium carbonate (544.5 mg, 2.37 mmol) and the reaction mixture was stirred at 120° C. for 2 h under nitrogen atmosphere. The resultant mixture was extracted with dichloromethane (20 mL*2), the combined organic layer was washed with water (10 mL*2), dried over sodium sulfate, and concentrated. The residue was subjected to silica gel column chromatography (2% methanol in dichloromethane) to obtain 2-(2-chloro-8-methyl-6-morpholino-9H-purin-9-yl)ethan-1-ol as white solid (180.0 mg, 76.9%). LCMS (ESI) m/z: 298.3 [M+H]⁺.

### Step 3: Synthesis of 2-(2-(3-methoxy-4-phenyl-1H-pyrazol-1-yl)-8-methyl-6-morpholino-9H-purin-9-yl)ethan-1-ol

To a solution of 2-(2-chloro-8-methyl-6-morpholino-9H-purin-9-yl)ethan-1-ol (150 mg, 0.50 mmol) in N,N-dimethylformamide (5 mL) were added 3-methoxy-4-phenyl-1H-pyrazole (96.5 mg, 0.55 mmol) and cesium carbonate (492.4 mg, 1.51 mmol). The resultant mixture was stirred at 120° C. for 2 h under nitrogen atmosphere. It was then extracted with dichloromethane (20 mL*2), the combined organic layer was washed with water (1 0 mL*2), dried over Na₂SO₄ and concentrated. The obtained residue was then subjected to silica gel column chromatography (1% methanol in dichloromethane) to obtain 2-(2-(3-methoxy-4-phenyl-1H-pyrazol-1-yl)-8-methyl-6-morpholino-9H-purin-9-yl)ethan-1-ol as white solid (109.3 mg, 49.8%). ¹H NMR (400 MHz, DMSO-d₆) δ 8.90 (s, 1H), 7.85-7.75 (m, 2H), 7.39 (t, J=7.7 Hz, 2H), 7.25 (d, J=7.4 Hz, 1H), 5.04 (s, 1H), 4.35-4.10 (m, 6H), 4.06 (s, 3H), 3.83-3.69 (m, 6H), 2.53 (s, 3H). LCMS (ESI) m/z: 436.2 [M+H]⁺.

### Synthesis of 1-(9-ethyl-2-(3-(1-methyl-1H-pyrazol-3-yl)phenyl)-6-(pyridin-4-yl)-9H-purin-8-yl)ethanol (Compound 449)

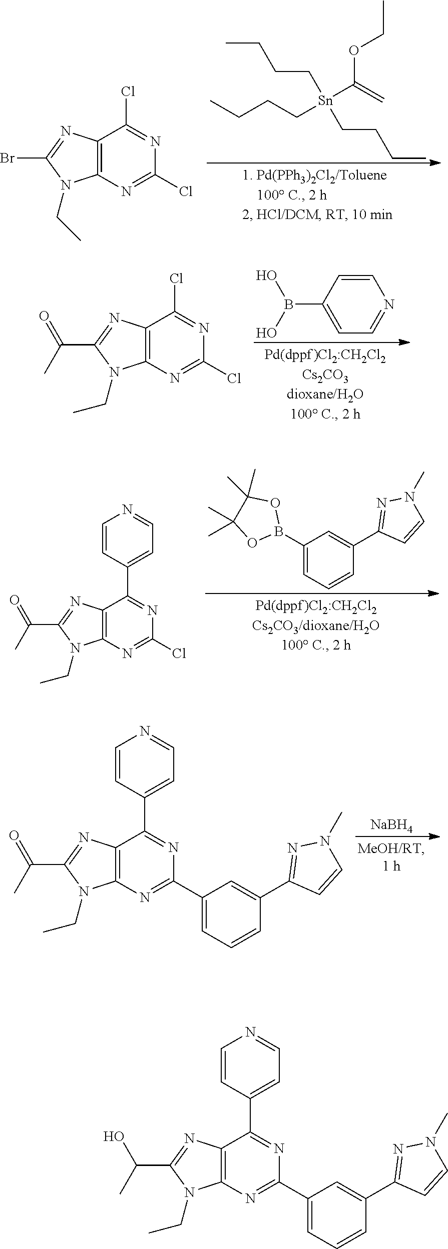

### Step 1: Synthesis of 1-(2,6-dichloro-9-ethyl-9H-purin-8-yl)ethenone

A mixture of 8-bromo-2,6-dichloro-9-ethyl-9H-purine (586 mg, 2.0 mmol), tributyl(1-ethoxyvinyl)stannane (724 mg, 2.0 mmol), bis(triphenylphosphine)palladium(II) chloride (140 mg, 0.2 mmol) in toluene (10 mL) was stirred at 100° C. under nitrogen atmosphere for 2 h. The mixture was poured into hydrochloric acid (1 mol/L, 20 mL) and stirred at 15° C. for another 1 h and then extracted with ethyl acetate (100 mL*2). The combined organic phase was concentrated and the residue was subjected to silica gel column chromatography (10% dichloromethane in methanol) to obtain the impure product which was further purified by prep-HPLC (Column Xbridge 21.2*250 mm C18, 10 um, Mobile Phase A: water (10 mmol/L ammonium bicarbonate) B: acetonitrile) to isolate 1-(2,6-dichloro-9-ethyl-9H-purin-8-yl)ethanone (110 mg, 21%) as grey solid. LCMS (ESI) m/z: 258.8/260.8 [M+H]⁺.

### Step 2: Synthesis of 1-(2-chloro-9-ethyl-6-(pyridin-4-yl)-9H-purin-8-yl)ethenone

A mixture of 1-(2,6-dichloro-9-ethyl-9H-purin-8-yl)ethanone (100 mg, 0.38 mmol), pyridin-4-ylboronic acid (46 mg, 0.38 mmol), 1,1′-bis(diphenylphosphino)ferrocene-palladium(II)dichloride dichloromethane complex (33 mg, 0.04 mmol), cesium carbonate (247 mg, 0.76 mmol) in 1,4-dioxane (8 mL) and water (0.5 mL) was stirred at 100° C. under nitrogen atmosphere for 2 h. The mixture was then poured into water, extracted with ethyl acetate (100 mL*2). The combined organic phase was concentrated and the residue was subjected to silica gel column chromatography (15% methanol in dichloromethane) to obtain 1-(2-chloro-9-ethyl-6-(pyridin-4-yl)-9H-purin-8-yl)ethanone (110 mg, 60.6%) as grey solid. LCMS (ESI) m/z: 301.8 [M+H]⁺.

### Step 3: Synthesis of 1-(9-ethyl-2-(3-(1-methyl-1H-pyrazol-3-yl)phenyl)-6-(pyridin-4-yl)-9H-purin-8-yl)ethenone

A mixture of 1-(2-chloro-9-ethyl-6-(pyridin-4-yl)-9H-purin-8-yl)ethanone (1.08 g, 5.0 mmol), 1-methyl-3-(3-(4,4,5,5-tetramethyl-1,3,2-dioxaborolan-2-yl)phenyl)-1H-pyrazole (102 mg, 0.36 mmol), 1,1′-bis(diphenylphosphino)ferrocene-palladium(II) dichloride dichloromethane complex (33 mg, 0.04 mmol) and cesium carbonate (234 mg, 0.72 mmol) in dioxane (8 mL) and water (1 mL) was stirred at 100° C. under nitrogen atmosphere for 2 h. The resultant mixture was poured into water and extracted with ethyl acetate (100 mL*2). The combined organic phase was concentrated and the residue was subjected to silica gel column chromatography (10% methanol in dichloromethane) to obtain 1-(9-ethyl-2-(3-(1-methyl-1H-pyrazol-3-yl)phenyl)-6-(pyridin-4-yl)-9H-purin-8-yl)ethanone (120 mg, 36%) as brown oil. LCMS (ESI) m/z: 423.5 [M+H]⁺.

### Step 4: Synthesis of 1-(9-ethyl-2-(3-(1-methyl-1H-pyrazol-3-yl)phenyl)-6-(pyridin-4-yl)-9H-purin-8-yl)ethanol

A mixture of 1-(9-ethyl-2-(3-(1-methyl-1H-pyrazol-3-yl)phenyl)-6-(pyridin-4-yl)-9H-purin-8-yl)ethanone (100 mg) and sodium borohydride (152 mg, 4.0 mmol) in methanol (5 mL) was stirred at 20° C. for 1 h. The mixture was concentrated and subjected to prep-HPLC (Column Xbridge 21.2*250 mm C18, 10 um, Mobile Phase A: water (10 mmol/L ammonium bicarbonate) B: acetonitrile) to obtain 1-(9-ethyl-2-(3-(1-methyl-1H-pyrazol-3-yl)phenyl)-6-(pyridin-4-yl)-9H-purin-8-yl)ethanol (11.8 mg, 0.027 mmol) as a white solid. ¹H NMR (400 MHz, CDCl₃) δ 8.97 (d, J=1.6 Hz, 1H), 8.86-8.82 (m, 2H), 8.82-8.78 (m, 2H), 8.56 (d, J=7.8 Hz, 1H), 7.93 (d, J=7.7 Hz, 1H), 7.54 (t, J=7.7 Hz, 1H), 7.45 (d, J=2.2 Hz, 1H), 6.68 (d, J=2.2 Hz, 1H), 5.20 (q, J=6.5 Hz, 1H), 4.48 (td, J=14.5, 7.1 Hz, 2H), 4.02 (s, 3H), 3.49 (bs, 1H), 1.80 (d, J=6.6 Hz, 3H), 1.59 (t, J=7.2 Hz, 3H); LCMS (ESI) m/z: 425.9 [M+H]⁺.

### Synthesis of 1-(9-ethyl-2-(3-(1-methyl-1H-pyrazol-3-yl)phenyl)-6-morpholino-9H-purin-8-yl)piperidin-4-ol (Compound 450)

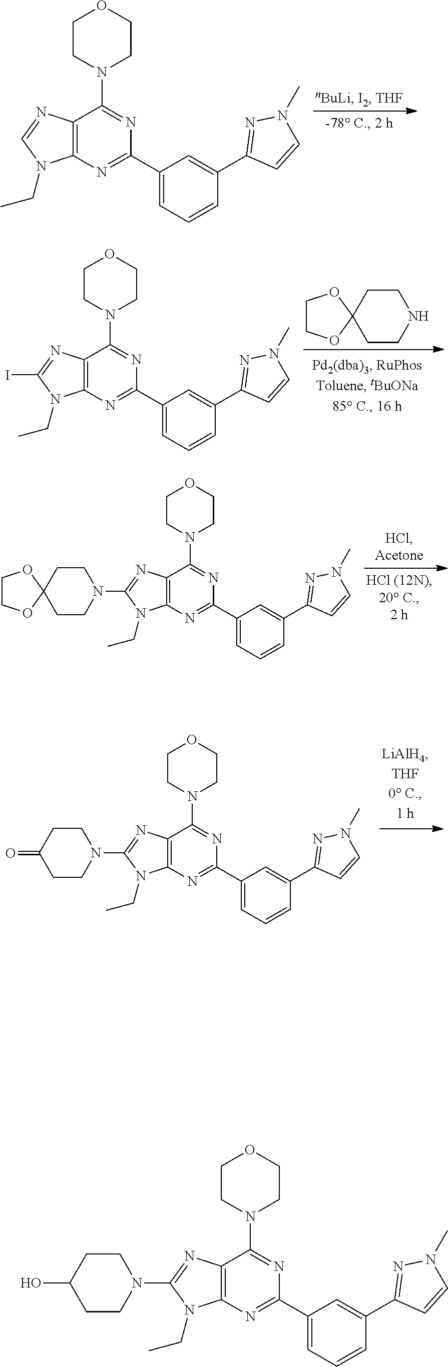

### Step 1: Synthesis of 4-(9-ethyl-8-iodo-2-(3-(1-methyl-1H-pyrazol-3-yl)phenyl)-9H-purin-6-yl)morpholine

To a stirred solution of 4-(9-ethyl-2-(3-(1-methyl-1H-pyrazol-3-yl)phenyl)-9H-purin-6-yl)morpholine (389 mg, 1 mmol) in tetrahydrofuran (20 mL) at −78° C. was added a solution of butyl lithium in tetrahydrofuran (2.5M, 0.44 mL, 1.1 mmol) and the reaction mixture was stirred for 30 minutes. Iodine (305 mg, 1.2 mmol) was then added and the reaction was stirred further at −78° C. for 2 h. It was then quenched by the addition of saturated aqueous ammonium chloride (10 mL) and then diluted with water (20 mL). The resultant mixture was extracted with ethyl acetate (50 mL*3), the combined organic layer was dried over sodium sulfate and concentrated. The residue was then subjected to silica gel chromatography eluting with a linear gradient of 0% to 3% methanol in dichloromethane to obtain 4-(9-ethyl-8-iodo-2-(3-(1-methyl-1H-pyrazol-3-yl)phenyl)-9H-purin-6-yl)morpholine (429 mg, 83%) as yellow solid. LCMS (ESI) m/z: 516.0 [M+H]⁺.

### Step 2: Synthesis of 8-(9-ethyl-2-(3-(1-methyl-1H-pyrazol-3-yl)phenyl)-6-morpholino-9H-purin-8-yl)-1,4-dioxa-8-azaspiro[4.5]decane

A solution of (4-(9-ethyl-8-iodo-2-(3-(1-methyl-1H-pyrazol-3-yl)phenyl)-9H-purin-6-yl)morpholine (206 mg, 0.4 mmol), 1,4-dioxa-8-azaspiro[4.5]decane (86 mg, 0.6 mmol), tris(dibenzylideneacetone)dipalladium (73 mg, 0.08 mmol), 2-dicyclohexylphosphino-2′,6′-diisopropoxybiphenyl (75 mg, 0.16 mmol) and sodium tert-butoxide (154 mg, 1.6 mmol) in toluene (20 mL) was stirred at 85° C. for 16 h under argon atmosphere. The reaction mixture was cooled, filtered to remove solids and the filtrate was concentrated. The residue was then subjected to prep-HPLC (BOSTON pHlex ODS 10 μm 21.2×250 mm 120 A. The mobile phase was acetonitrile/0.1% ammonium bicarbonate) to obtain 8-(9-ethyl-2-(3-(1-methyl-1H-pyrazol-3-yl)phenyl)-6-morpholino-9H-purin-8-yl)-1,4-dioxa-8-azaspiro[4.5]decane (80 mg, 38%) as yellow solid. LCMS (ESI) m/z: 531.3 [M+H]⁺.

### Step 3: Synthesis of 1-(9-ethyl-2-(3-(1-methyl-1H-pyrazol-3-yl)phenyl)-6-morpholino-9H-purin-8-yl)piperidin-4-one

A mixture of 8-(9-ethyl-2-(3-(1-methyl-1H-pyrazol-3-yl)phenyl)-6-morpholino-9H-purin-8-yl)-1,4-dioxa-8-azaspiro[4.5]decane (80 mg, 0.15 mmol) in concentrated hydrochloric acid (1 mL) and acetone (2 mL) was stirred at 20° C. for 2 h. It was concentrated and the residue was diluted with water (30 mL), the pH was adjusted to 8 with saturated sodium bicarbonate and extracted with dichloromethane (50 mL×2).

The organic phase was washed with brine (50 mL), dried over sodium sulfate, filtered and concentrated.

The residue was subjected to silica gel column chromatography (ethyl acetate/petroleum=0%-50%) to obtain 1-(9-ethyl-2-(3-(1-methyl-1H-pyrazol-3-yl)phenyl)-6-morpholino-9H-purin-8-yl)piperidin-4-one (57 mg, 78%) as yellow solid. LCMS (ESI) m/z: 487.2 [M+H]⁺.

### Step 4: Synthesis of 1-(9-ethyl-2-(3-(1-methyl-1H-pyrazol-3-yl)phenyl)-6-morpholino-9H-purin-8-yl)piperidin-4-ol

To a solution of 1-(9-ethyl-2-(3-(1-methyl-1H-pyrazol-3-yl)phenyl)-6-morpholino-9H-purin-8-yl)piperidin-4-one (90 mg, 0.185 mmol) in anhydrous tetrahydrofuran (15 mL) was added a solution of lithium aluminum hydride (1 M in tetrahydrofuran, 0.74 mL, 0.74 mmol,) at 0° C. and the resultant mixture was stirred under nitrogen for 1 h. It was then quenched by the careful addition of sodium sulfate decahydrate with ice-bath cooling. The solids were filtered-off and the filtrate was evaporated in vacuo. The residue was subjected to prep-HPLC (BOSTON pHlex ODS 10 μm 21.2×250 mm 120 A. The mobile phase was acetonitrile/0.1% ammonium bicarbonate) to obtain 1-(9-ethyl-2-(3-(1-methyl-1H-pyrazol-3-yl)phenyl)-6-morpholino-9H-purin-8-yl)piperidin-4-ol (39.8 mg, 44%) as white solid. ¹H NMR (500 MHz, DMSO-d₆) δ 8.73 (s, 1H), 8.28 (d, J=7.8 Hz, 1H), 7.82 (d, J=7.7 Hz, 1H), 7.77 (d, J=2.1 Hz, 1H), 7.47 (t, J=7.7 Hz, 1H), 6.72 (d, J=2.2 Hz, 1H), 4.77 (d, J=4.2 Hz, 1H), 4.22 (s, 4H), 4.15 (q, J=7.1 Hz, 2H), 3.92 (s, 3H), 3.82-3.74 (m, 4H), 3.73-3.67 (m, 1H), 3.51-3.42 (m, 2H), 3.09-2.94 (m, 2H), 1.98-1.81 (m, 2H), 1.65-1.53 (m, 2H), 1.45 (t, J=7.2 Hz, 3H). LCMS (ESI) m/z: 488.9[M+H]⁺.

### Synthesis of enantiomer 1 (Compound 451) and enantiomer 2 (Compound 452) of (4-(9-ethyl-2-(4-phenyl-1H-pyrazol-1-yl)-8-(pyridin-4-yl)-9H-purin-6-yl)morpholin-3-yl)methanol

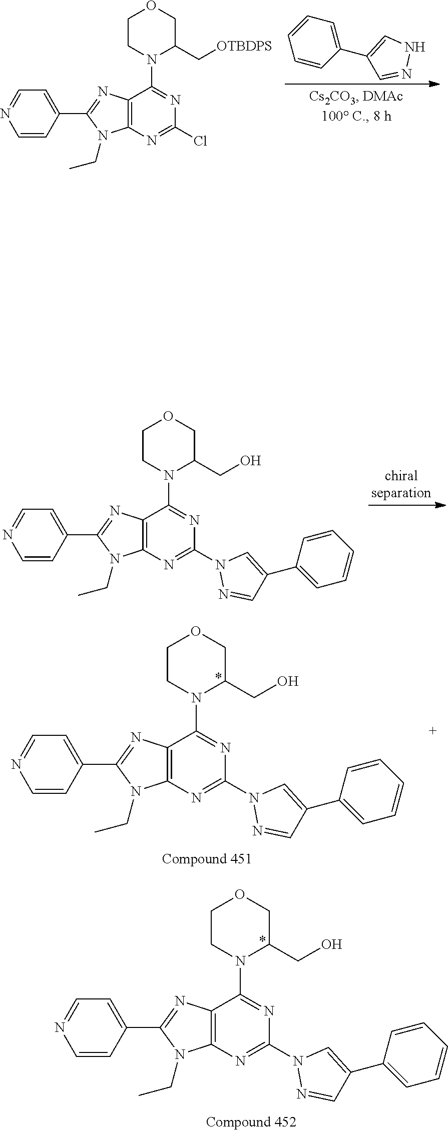

A mixture of 3-((tert-butyldiphenylsilyloxy)methyl)-4-(2-chloro-9-ethyl-8-(pyridin-4-yl)-9H-purin-6-yl)morpholine (300 mg, 0.49 mmol), 4-phenyl-1H-pyrazole (78 mg, 0.54 mmol) and cesium carbonate (318 mg, 0.98 mmol) in N,N-dimethylacetamide (15 mL) was stirred at 100° C. for 8 h. The mixture was diluted with ethyl acetate (55 mL) and washed with water (55 mL). The organic layer was dried over sodium sulfate, filtered and concentrated under the reduced pressure. The crude product was purified by HPLC (BOSTON pHlex ODS 10 μm 21.2×250 mm 120 A. The mobile phase was acetonitrile/0.1% ammonium bicarbonate) to obtain (4-(9-ethyl-2-(4-phenyl-1H-pyrazol-1-yl)-8-(pyridin-4-yl)-9H-purin-6-yl)morpholin-3-yl)methanol (150 mg, 63%) as white solid. This achiral product was subjected to chiral-HPLC (Instrument: SFC-150 (Waters) Column: OJ 20*250 mm, 10 um (Daicel) Column temperature: 35° C. Mobile phase: CO₂/MeOH (0.2% methanol ammonia)=65/35, Flow rate: 120 g/min, Back pressure: 100 bar, Detection wavelength: 214 nm, Cycle time: 3.5 min Sample solution: 99 mg dissolved in 10 ml methanol Injection volume: 2 ml) to obtain two enantiomers: Compound 113 (65.6 mg, 0.14 mmol) and compound 114 (64.7 mg, 0.13 mmol)) were isolated as white solids.

Compound 113: ¹H NMR (400 MHz, DMSO-d₆) δ 9.07 (s, 1H), 8.80 (dd, J=4.5, 1.5 Hz, 2H), 8.27 (d, J=0.6 Hz, 1H), 7.86 (d, J=5.1 Hz, 2H), 7.79 (d, J=7.4 Hz, 2H), 7.43 (t, J=7.7 Hz, 2H), 7.28 (t, J=7.4 Hz, 1H), 5.93-5.53 (m, 1H), 4.97 (s, 2H), 4.45 (q, J=7.0 Hz, 2H), 4.11 (s, 1H), 4.01 (d, J=8.1 Hz, 1H), 3.92-3.83 (m, 1H), 3.78-3.52 (m, 4H), 1.36 (t, J=7.2 Hz, 3H). LCMS: (ESI) m/z: 482.7 [M+H]⁺; (RT: 1.373 min).

Compound 114: ¹H NMR (400 MHz, DMSO-d₆) δ 9.07 (s, 1H), 8.80 (dd, J=4.5, 1.5 Hz, 2H), 8.27 (d, J=0.6 Hz, 1H), 7.86 (d, J=5.1 Hz, 2H), 7.79 (d, J=7.4 Hz, 2H), 7.43 (t, J=7.7 Hz, 2H), 7.28 (t, J=7.4 Hz, 1H), 5.93-5.53 (m, 1H), 4.97 (s, 2H), 4.45 (q, J=7.0 Hz, 2H), 4.11 (s, 1H), 4.01 (d, J=8.1 Hz, 1H), 3.92-3.83 (m, 1H), 3.78-3.52 (m, 4H), 1.36 (t, J=7.2 Hz, 3H). LCMS: (ESI) m/z: 482.7 [M+H]⁺; (RT: 2.033 min).

### Synthesis of 3-(9-ethyl-2-(3-methoxy-4-phenyl-1H-pyrazol-1-yl)-6-morpholino-9H-purin-8-yl)propan-1-ol (Compound 453)

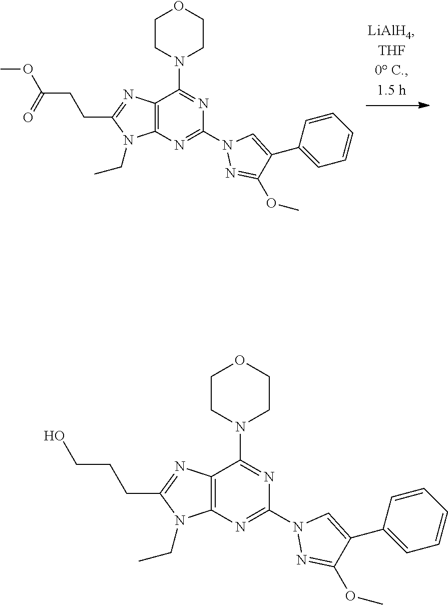 [SMILES: CCn1c(CCC(=O)OC)nc2c(N3CCOCC3)nc(-n3cc(-c4ccccc4)c(OC)n3)nc21 ; CCn1c(CCCO)nc2c(N3CCOCC3)nc(-n3cc(-c4ccccc4)c(OC)n3)nc21]

Lithium aluminum hydride (0.41 mL, 0.41 mmol) in anhydrous tetrahydrofuran was added to a solution of methyl 3-(9-ethyl-2-(3-methoxy-4-phenyl-1H-pyrazol-1-yl)-6-morpholino-9H-purin-8-yl)propanoate (100 mg, 0.2 mmol) in anhydrous tetrahydrofuran (5 mL) at 0° C. and the resultant mixture was stirred under nitrogen for 1.5 h. The reaction mixture with was then quenched by the careful addition of sodium sulfate decahydrate with ice bath cooling. Tetrahydrofuran (50 mL) was added to the reaction mixture and stirred for 15 min. The mixture was then filtered to remove solids and the solids washed with tetrahydrofuran (50 mL). The combined filtrates were evaporated in vacuo and the residue was subjected to prep-HPLC (BOSTON pHlex ODS 10 μm 21.2×250 mm 120 A, with mobile phase acetonitrile/0.1% ammonium bicarbonate) to obtain 3-(9-ethyl-2-(3-methoxy-4-phenyl-1H-pyrazol-1-yl)-6-morpholino-9H-purin-8-yl)propan-1-ol (16.8 mg, 18%) as off-white solid. ¹H NMR (400 MHz, DMSO-d₆) δ 8.91 (s, 1H), 7.84-7.77 (m, 2H), 7.39 (t, J=7.8 Hz, 2H), 7.24 (t, J=7.4 Hz, 1H), 4.58 (s, 1H), 4.29 (bs, 4H), 4.21 (d, J=7.3 Hz, 2H), 4.07 (s, 3H), 3.80-3.73 (m, 4H), 3.53 (t, J=6.2 Hz, 2H), 2.91-2.85 (m, 2H), 1.96-1.87 (m, 2H), 1.35 (t, J=7.2 Hz, 3H). LCMS (ESI) m/z: 463.8 [M+H]⁺.

### Synthesis of 4-(2-(3-(1H-pyrazol-1-yl)phenyl)-9-ethyl-6-morpholino-9H-purin-8-yl)-1-methylpiperazin-2-one (Compound 454)

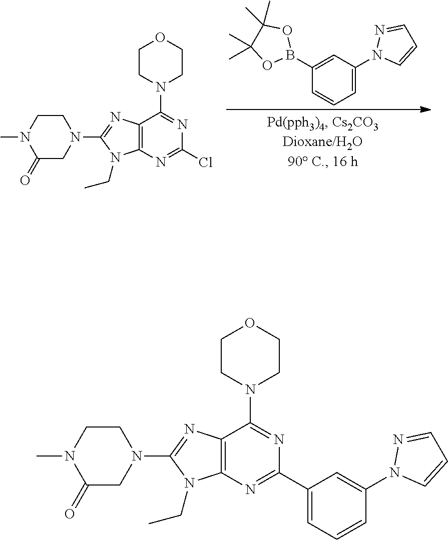 [SMILES: CCn1c(N2CCN(C)C(=O)C2)nc2c(N3CCOCC3)nc(Cl)nc21 ; CC1(C)OB(c2cccc(-n3cccn3)c2)OC1(C)C ; CCn1c(N2CCN(C)C(=O)C2)nc2c(N3CCOCC3)nc(-c3cccc(-n4cccn4)c3)nc21]

A solution of 4-(2-chloro-9-ethyl-6-morpholino-9H-purin-8-yl)-1-methylpiperazin-2-one (42 mg, 0.111 mmol), 1-(3-(4,4,5,5-tetramethyl-1,3,2-dioxaborolan-2-yl)phenyl)-1H-pyrazole (34 mg, 0.126 mmol), cesium carbonate (68 mg, 0.21 mmol) and tetrakis(triphenylphosphine)palladium (9 mg, 0.008 mmol) in 1,4-dioxane/water (5 mL/0.5 mL) was stirred at 90° C. for 16 h under argon atmosphere. The resultant mixture was filtered to remove solids, the filtrate was concentrated and the residue was subjected to prep-HPLC (BOSTON pHlex ODS 10 μm 21.2×250 mm 120 A. The mobile phase was acetonitrile/0.1% ammonium bicarbonate) to obtain 4-(2-(3-(1H-pyrazol-1-yl)phenyl)-9-ethyl-6-morpholino-9H-purin-8-yl)-1-methylpiperazin-2-one (10.6 mg, 25.36%) as white solid. ¹H NMR (400 MHz, DMSO-d₆) δ 8.76 (s, 1H), 8.56 (d, J=2.4 Hz, 1H), 8.31 (d, J=7.9 Hz, 1H), 7.88 (d, J=7.9 Hz, 1H), 7.80 (d, J=1.6 Hz, 1H), 7.58 (t, J=7.9 Hz, 1H), 6.71-6.46 (m, 1H), 4.25 (bs, 4H), 4.22 (d, J=7.1 Hz, 2H), 3.90 (s, 2H), 3.79-3.75 (m, 4H), 3.56 (d, J=5.4 Hz, 2H), 3.49 (d, J=5.4 Hz, 2H), 2.91 (s, 3H), 1.44 (t, J=7.1 Hz, 3H). LCMS (ESI) m/z: 488.3 [M+H]⁺.

### Synthesis of 1-(9-ethyl-6-morpholino-2-(3-(pyridazin-3-yl)phenyl)-9H-purin-8-yl)ethan-1-ol (Compound 455)

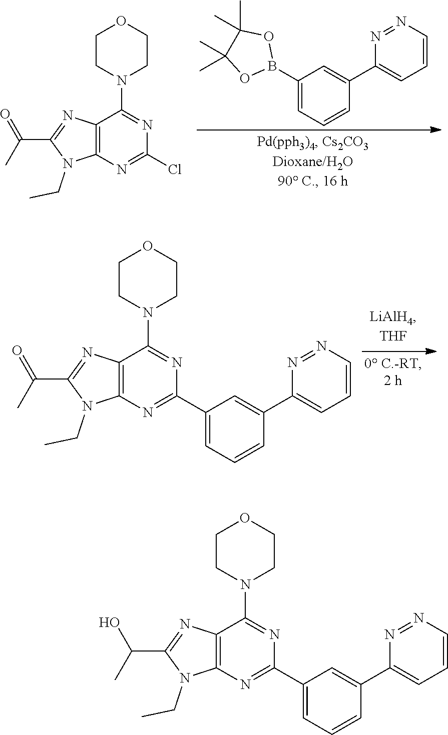 [SMILES: CCn1c(C(C)=O)nc2c(N3CCOCC3)nc(Cl)nc21 ; CC1(C)OB(c2cccc(-c3cccnn3)c2)OC1(C)C ; CCn1c(C(C)=O)nc2c(N3CCOCC3)nc(-c3cccc(-c4cccnn4)c3)nc21 ; CCn1c(C(C)O)nc2c(N3CCOCC3)nc(-c3cccc(-c4cccnn4)c3)nc21]

### Step 1: Synthesis of 1-(9-ethyl-6-morpholino-2-(3-(pyridazin-3-yl)phenyl)-9H-purin-8-yl)ethan-1-one

A mixture of 1-(2-chloro-9-ethyl-6-morpholino-9H-purin-8-yl)ethan-1-one (100 mg, 0.32 mmol), 3-(3-(4,4,5,5-tetramethyl-1,3,2-dioxaborolan-2-yl)phenyl)pyridazine (100 mg, 0.35 mmol), cesium carbonate (375 mg, 1.05 mmol) and tetrakis(triphenyl phosphine)palladium (35 mg, 0.03 mmol) in 1,4-dioxane/water (20 mL/2 mL) was stirred at 90° C. for 16 h under argon atmosphere. The mixture was then filtered to remove the solids, the filtrate was concentrated and the residue was subjected to silica gel chromatography (eluting with 0% to 53% ethyl acetate in petroleum ether) to obtain 1-(9-ethyl-6-morpholino-2-(3-(pyridazin-3-yl)phenyl)-9H-purin-8-yl)ethan-1-one (113 mg, 67.78%) as white solid. LCMS (ESI) m/z: 430.2 [M+H]⁺.

### Step 2: Synthesis of 1-(9-ethyl-6-morpholino-2-(3-(pyridazin-3-yl)phenyl)-9H-purin-8-yl)ethan-1-ol

A solution of 1-(9-ethyl-6-morpholino-2-(3-(pyridazin-3-yl)phenyl)-9H-purin-8-yl)ethan-1-one (93 mg, 0.217 mmol)) and lithium aluminum hydride (1.6M solution in THF, 0.63 ml, 0.44 mmol) in dry tetrahydrofuran (5 mL) was stirred at room temperature for 2 h. The reaction mixture was filtered, the filtrate was concentrated and the residue was subjected to prep-HPLC (BOSTON pHlex ODS 10 μm 21.2×250 mm 120 A. The mobile phase was acetonitrile/0.1% ammonium bicarbonate) to obtain 1-(9-ethyl-6-morpholino-2-(3-(pyridazin-3-yl)phenyl)-9H-purin-8-yl)ethan-1-ol (60.9 mg, 54.06%) as white solid. ¹H NMR (400 MHz, DMSO-d₆) δ 9.27 (d, J=3.9 Hz, 1H), 9.14 (s, 1H), 8.57 (d, J=7.8 Hz, 1H), 8.29 (d, J=8.6 Hz, 1H), 8.19 (d, J=7.7 Hz, 1H), 7.84 (dd, J=8.6, 4.9 Hz, 1H), 7.68 (t, J=7.8 Hz, 1H), 5.72 (d, J=6.2 Hz, 1H), 5.05 (p, J=6.4 Hz, 1H), 4.42 (q, J=7.1 Hz, 2H), 4.33 (bs, 4H), 3.84-3.75 (m, 4H), 1.59 (d, J=6.5 Hz, 3H), 1.45 (t, J=7.1 Hz, 3H). LCMS (ESI) m/z: 431.8 [M+H]⁺.

### Synthesis of 4-(9-ethyl-2-(3-(1-methyl-1H-pyrazol-3-yl)phenyl)-6-morpholino-9H-purin-8-yl)-1-methylpiperidin-2-one (Compound 456)

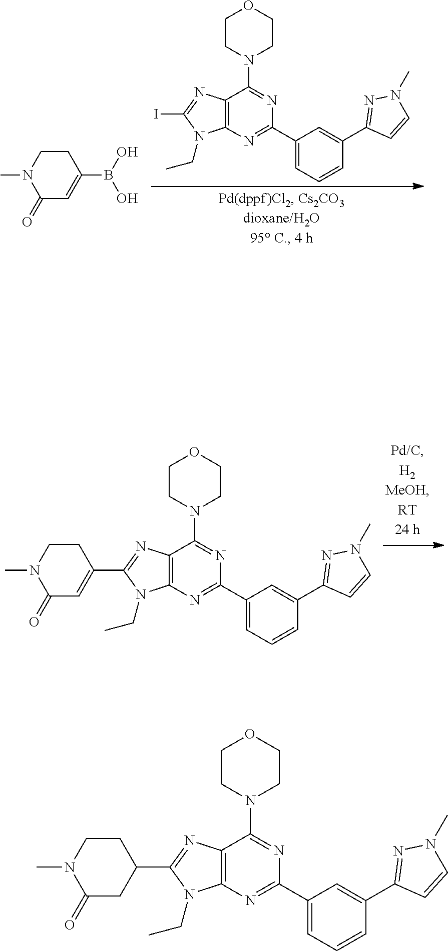

### Step 1: Synthesis of 4-(9-ethyl-2-(3-(1-methyl-1H-pyrazol-3-yl)phenyl)-6-morpholino-9H-purin-8-yl)-1-methyl-5,6-dihydropyridin-2(1H)-one

To a solution ′f (1-methyl-6-oxo-1,2,3,6-tetrahydropyridin-4-yl)boronic acid (0.09 g, 0.58 mmol) in 1,4-dioxane (5 mL) were added 4-(9-ethyl-8-iodo-2-(3-(1-methyl-1H-pyrazol-3-yl)phenyl)-9H-purin-6-yl)morpholine (0.25 g, 0.49 mmol), cesium carbonate (0.4 g, 1.2 mmol), [1,1′-bis(diphenylphosphino)ferrocene]dichloropalladium(II) (0.035 g, 0.05 mmol) and water (1.5 mL). The resultant reaction mixture was stirred at 95° C. for 4 h under nitrogen atmosphere. The reaction mixture was then filtered, the filtrate was concentrated and the residue was subjected to flash chromatography (dichloromethane:methanol=15:1) to obtain 4-(9-ethyl-2-(3-(1-methyl-1H-pyrazol-3-yl)phenyl)-6-morpholino-9H-purin-8-yl)-1-methyl-5,6-dihydropyridin-2(1H)-one (0.2 g, 83%) as yellow solid. LCMS (ESI) m/z: 499.1 [M+H]⁺.

### Step 2: Synthesis of 4-(9-ethyl-2-(3-(1-methyl-1H-pyrazol-3-yl)phenyl)-6-morpholino-9H-purin-8-yl)-1-methylpiperidin-2-one

A mixture of 4-(9-ethyl-2-(3-(1-methyl-1H-pyrazol-3-yl)phenyl)-6-morpholino-9H-purin-8-yl)-1-methyl-5,6-dihydropyridin-2(1H)-one (200 mg, 0.4 mmol) and palladium on activated carbon 10% (100 mg) in methanol (10 mL) was stirred at 25° C. for 16 h under hydrogen atmosphere. The reaction mixture was filtered to remove the solids, the filtrate was concentrated and the residue was subjected to prep-HPLC (BOSTON pHlex ODS 10 μm 21.2×250 mm 120 A, with mobile phase acetonitrile/0.1% ammonium bicarbonate) to obtain 4-(9-ethyl-2-(3-(1-methyl-1H-pyrazol-3-yl)phenyl)-6-morpholino-9H-purin-8-yl)-1-methylpiperidin-2-one (48.3 mg, 24%) as white solid. ¹H NMR (400 MHz, MeOD-d₄) δ 8.80 (d, J=1.6 Hz, 1H), 8.36 (d, J=7.9 Hz, 1H), 7.80 (d, J=7.8 Hz, 1H), 7.64 (d, J=2.3 Hz, 1H), 7.47 (t, J=7.7 Hz, 1H), 6.68 (d, J=2.3 Hz, 1H), 4.46-4.26 (m, 6H), 3.97 (s, 3H), 3.86-3.80 (m, 4H), 3.72-3.59 (m, 1H), 3.48 (ddd, J=17.6, 11.3, 7.3 Hz, 1H), 3.29-3.26 (m, 1H), 2.97 (s, 3H), 2.84 (dd, J=17.3, 7.6 Hz, 1H), 2.72 (dd, J=17.2, 5.5 Hz, 1H), 2.26 (s, 1H), 2.14 (dd, J=13.8, 5.5 Hz, 1H), 1.50 (t, J=7.2 Hz, 3H). LCMS (ESI) m/z: 501.1 [M+H]⁺.

### Synthesis of enantiomer 1 (Compound 457) and enantiomer 2 (Compound 458) of 4-(9-ethyl-2-(3-(1-methyl-1H-pyrazol-3-yl)phenyl)-6-morpholino-9H-purin-8-yl)-1-methylpiperidin-2-one

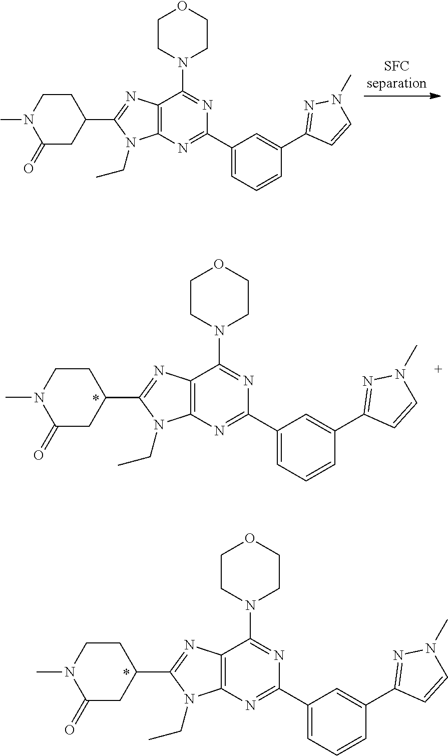

The racemic compound (39 mg, 0.078 mmol) was resolved by chiral prep-HPLC (Instrument: SFC-80 (Thar, Waters), Column: AD 20*250 mm, 10 um (Daicel), Column temperature: 35° C., Mobile phase: carbon dioxide/ethanol (0.5% methanol ammonia)=65/35, Flow rate: 80 g/min, Back pressure: 100 bar, Detection wavelength: 214 nm, Cycle time: 3.0 min, Sample solution: 200 mg dissolved in 25 mL Methanol, Injection volume: 1.0 mL) to obtain two enantiomers: enantiomer 1 (14.7 mg, 38%) and enantiomer 2 (15.2 mg, 39%) as off-white solids.

Compound 119: ¹H NMR (400 MHz, DMSO-d₆) δ 8.74 (t, J=1.6 Hz, 1H), 8.30 (dd, J=5.3, 3.9 Hz, 1H), 7.84 (dd, J=5.2, 3.8 Hz, 1H), 7.77 (d, J=2.2 Hz, 1H), 7.49 (t, J=7.7 Hz, 1H), 6.74 (d, J=2.2 Hz, 1H), 4.42-4.19 (m, 6H), 3.92 (s, 3H), 3.79-3.72 (m, 4H), 3.67-3.59 (m, 1H), 3.48-3.42 (m, 1H), 3.28-3.25 (m, 1H), 2.86 (s, 3H), 2.62 (dd, J=11.6, 7.3 Hz, 2H), 2.17 (d, J=9.0 Hz, 1H), 2.00 (dd, J=8.9, 4.8 Hz, 1H), 1.41 (t, J=7.2 Hz, 3H). LCMS (ESI) m/z: 500.7 [M+H]⁺; (RT: 1.84 min.).

Compound 120: ¹H NMR (400 MHz, DMSO-d₆) δ 8.74 (t, J=1.6 Hz, 1H), 8.30 (dd, J=5.3, 3.9 Hz, 1H), 7.84 (dd, J=5.2, 3.8 Hz, 1H), 7.77 (d, J=2.2 Hz, 1H), 7.49 (t, J=7.7 Hz, 1H), 6.74 (d, J=2.2 Hz, 1H), 4.42-4.19 (m, 6H), 3.92 (s, 3H), 3.79-3.72 (m, 4H), 3.67-3.59 (m, 1H), 3.48-3.42 (m, 1H), 3.28-3.25 (m, 1H), 2.86 (s, 3H), 2.62 (dd, J=11.6, 7.3 Hz, 2H), 2.17 (d, J=9.0 Hz, 1H), 2.00 (dd, J=8.9, 4.8 Hz, 1H), 1.41 (t, J=7.2 Hz, 3H). LCMS (ESI) m/z: 500.8 [M+H]⁺; (RT: 2.57 min.).

### Synthesis of 1-[3-[9-ethyl-6-morpholino-8-(4-pyridyl)purin-2-yl]phenyl]piperidin-4-ol (Compound 459)

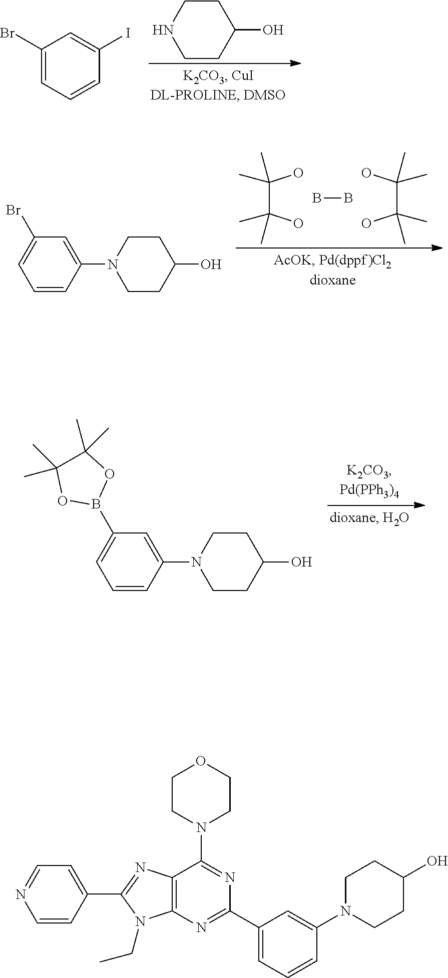

### Step 1: Synthesis of 1-(3-bromophenyl)piperidin-4-ol

To a solution of 1-bromo-3-iodo-benzene (1 g, 3.53 mmol) in DMSO (7 mL) were added piperidin-4-ol (298 mg, 2.95 mmol), K₂CO₃ (1.22 g, 8.84 mmol), DL-PROLINE (136 mg, 1.18 mmol) and CuI (224 mg, 1.18 mmol). The resultant reaction mixture was heated to 90° C., stirred for 12 h under nitrogen atmosphere. The mixture was filtered to remove the solids and filtrate was diluted with 10 mL of water. The filtrate was then extracted with ethyl acetate (10 mL*2), the combined organic layer was washed with brine (10 mL), dried over Na₂SO₄ and concentrated. The residue was then subjected to flash column chromatography (ISCO 20 g silica, 0-26% ethyl acetate in petroleum ether, gradient over 20 min) to obtain 1-(3-bromophenyl)piperidin-4-ol (550 mg, 55%) as white solid. ¹H NMR (400 MHz, CHLOROFORM-d) δ 7.21-7.02 (m, 2H), 7.02-6.75 (m, 2H), 3.89 (bs, 1H), 3.63-3.47 (m, 2H), 2.97 (t, J=9.6 Hz, 2H), 2.06-2.00 (m, 1H), 1.77-1.43 (m, 4H); LCMS (ESI) m/z: 256.0 [M+H]⁺.

### Step 2: Synthesis of 1-[3-(4,4,5,5-tetramethyl-1,3,2-dioxaborolan-2-yl)phenyl]piperidin-4-ol

To a solution of 1-(3-bromophenyl)piperidin-4-ol (500 mg, 1.48 mmol) in dioxane (7 mL) were added 4,4,5,5-tetramethyl-2-(4,4,5,5-tetramethyl-1,3,2-dioxaborolan-2-yl)-1,3,2-dioxaborolane (753 mg, 2.97 mmol), AcOK (437 mg, 4.45 mmol) and Pd(dppf)Cl₂ (54 mg, 74 umol). The reaction mixture was heated to 100° C. and stirred for 12 h under nitrogen atmosphere. The reaction mixture was filtered and the filtrate was concentrated. The residue was diluted with 10 mL of water and was extracted with ethyl acetate (10 mL*2). The combined organic layer was washed with brine (10 mL), dried over Na₂SO₄ and concentrated. The residue was subjected to flash column chromatography (ISCO 20 g silica, 0-37% ethyl acetate in petroleum ether, gradient over 20 min) to obtain 1-[3-(4,4,5,5-tetramethyl-1,3,2-dioxaborolan-2-yl)phenyl]piperidin-4-ol (500 mg, 89%) as pale yellow solid. ¹H NMR (400 MHz, CHLOROFORM-d) δ 7.33 (d, J=1.6 Hz, 1H), 7.19 (s, 2H), 6.98 (d, J=7.0 Hz, 1H), 3.82-3.72 (m, 1H), 3.58-3.47 (m, 2H), 2.91-2.79 (m, 2H), 1.98 (s, 2H), 1.68-1.57 (m, 2H), 1.26 (s, 12H); LCMS (ESI) m/z: 304.2 [M+H]⁺.

### Step 3: Synthesis of 1-[3-[9-ethyl-6-morpholino-8-(4-pyridyl)purin-2-yl]phenyl]piperidin-4-ol

To a solution of 1-[3-(4,4,5,5-tetramethyl-1,3,2-dioxaborolan-2-yl)phenyl]piperidin-4-ol (176 mg, 580 umol) in dioxane (1 mL) and H₂O (0.1 mL) were added 4-[2-chloro-9-ethyl-8-(4-pyridyl)purin-6-yl]morpholine (100 mg, 290 umol), Pd(PPh₃)₄ (34 mg, 29 umol) and K₂CO₃ (120 mg, 870 umol, 3 eq). The mixture was stirred at 100° C. for 12 h under nitrogen atmosphere. It was filtered to remove the solids and the filtrate was concentrated. The resultant residue was diluted with 3 mL of water and it was extracted with ethyl acetate (3 mL*2). The combined organic layers was washed with brine (3 mL), dried over Na₂SO₄ and concentrated. The residue was then subjected to prep-HPLC (Phenomenex Luna 80*30 mm*3 um column; 1-25% acetonitrile in an a 0.05% hydrochloric acid solution in water, 8 min gradient) to obtain 1-[3-[9-ethyl-6-morpholino-8-(4-pyridyl)purin-2-yl]phenyl]piperidin-4-ol (140 mg, 88%) as yellow solid. ¹H NMR (400 MHz, METHANOL-d4) δ 8.99 (d, J=6.6 Hz, 2H), 8.91 (s, 1H), 8.72 (d, J=7.8 Hz, 1H), 8.56 (d, J=6.9 Hz, 2H), 8.22-8.18 (m, 1H), 7.86 (dd, J=2.0, 8.0 Hz, 1H), 4.75 (q, J=7.1 Hz, 2H), 4.53 (bs, 4H), 4.18 (bs, 1H), 4.00-3.85 (m, 6H), 3.75 (d, J=2.0 Hz, 2H), 2.42-2.06 (m, 4H), 1.60 (t, J=7.2 Hz, 3H). LCMS (ESI) for (C27H31N7O2) [M+H]⁺: 486.2.

### Synthesis of 4-[3-[9-ethyl-6-morpholino-8-(4-pyridyl)purin-2-yl]phenyl]-1-methyl-piperazin-2-one (Compound 460)

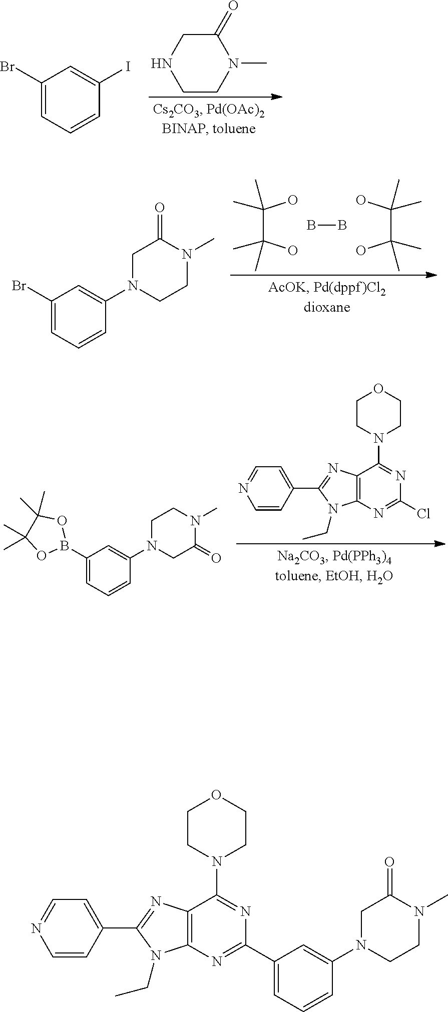

### Step 1: Synthesis of 4-(3-bromophenyl)-1-methyl-piperazin-2-one

To a solution of 1-bromo-3-iodo-benzene (1 g, 3.53 mmol) in toluene (10 mL) were added 1-methylpiperazin-2-one (336 mg, 2.95 mmol), Cs₂CO₃ (3.84 g, 11.78 mmol), BINAP (367 mg, 589 umol) and Pd(OAc)₂ (132 mg, 589 umol). The reaction mixture was heated to 100° C. and stirred for 12 h under nitrogen atmosphere. The mixture was filtered, the filtrate was diluted with 10 mL of water and extracted with ethyl acetate (15 mL*2). The combined organic layers was washed with brine (15 mL) and dried over Na₂SO₄ and concentrated. The residue was subjected to flash column chromatography (ISCO 20 g silica, 0-46% ethyl acetate in petroleum ether, gradient over 20 min) to obtain 4-(3-bromophenyl)-1-methyl-piperazin-2-one (450 mg, 57%) as yellow solid. ¹H NMR (400 MHz, CHLOROFORM-d) δ 7.10-7.03 (m, 1H), 6.97-6.88 (m, 2H), 6.70 (ddd, J=8.4, 2.4, 0.8 Hz, 1H), 3.79 (s, 2H), 3.44-3.37 (m, 4H), 2.97 (s, 3H); LCMS (ESI) m/z: 269.0 [M+H]⁺.

### Step 2: Synthesis of 1-methyl-4-[3-(4,4,5,5-tetramethyl-1,3,2-dioxaborolan-2-yl)phenyl]piperazin-2-one

To a solution of 4-(3-bromophenyl)-1-methyl-piperazin-2-one (400 mg, 1.49 mmol) in dioxane (5 mL) were added 4,4,5,5-tetramethyl-2-(4,4,5,5-tetramethyl-1,3,2-dioxaborolan-2-yl)-1,3,2-dioxaborolane (755 mg, 2.97 mmol), KOAc (438 mg, 4.46 mmol) and Pd(dppf)Cl₂ (54 mg, 74 umol). The resultant reaction mixture was heated to 100° C. and stirred for 12 h under nitrogen atmosphere. The mixture was filtered and the filtrate was concentrated. The residue was further diluted with 5 mL of water and the mixture was extracted with ethyl acetate (5 mL*2). The combined organic layers was washed with brine (5 mL), dried over Na₂SO₄ and concentrated. The residue was then subjected to flash column chromatography (ISCO 20 g silica, 0-52% ethyl acetate in petroleum ether, gradient over 20 min) to obtain 1-methyl-4-[3-(4,4,5,5-tetramethyl-1,3,2-dioxaborolan-2-yl)phenyl]piperazin-2-one (300 mg, 64%) as yellow solid. ¹H NMR (400 MHz, CHLOROFORM-d) δ 7.30-7.17 (m, 3H), 6.94-6.88 (m, 1H), 3.82 (s, 2H), 3.48-3.35 (m, 4H), 2.97 (s, 3H), 1.27 (s, 12H); LCMS (ESI) m/z: 317.1 [M+H]⁺.

### Step 3: Synthesis of 4-[3-[9-ethyl-6-morpholino-8-(4-pyridyl)purin-2-yl]phenyl]-1-methyl-piperazin-2-one

To a solution of 1-methyl-4-[3-(4,4,5,5-tetramethyl-1,3,2-dioxaborolan-2-yl)phenyl]piperazin-2-one (37 mg, 116 umol) in toluene (2 mL), EtOH (2 mL) and H₂O (1 mL) were added 4-[2-chloro-9-ethyl-8-(4-pyridyl)purin-6-yl]morpholine (20 mg, 58 umol), Na₂CO₃ (9 mg, 87 umol) and Pd(PPh₃)₄ (7 mg, 6 umol). The resultant mixture was stirred at 90° C. for 2 h under nitrogen atmosphere, then filtered and the filtrate was concentrated. The residue was subjected to prep-HPLC (Waters Xbridge BEH C18 100*30 mm*10 um column; 20-50% acetonitrile in an a 10 mM ammonium hydroxide solution in water, 8 min gradient) to obtain 4-[3-[9-ethyl-6-morpholino-8-(4-pyridyl)purin-2-yl]phenyl]-1-methyl-piperazin-2-one (1 mg, 2%) as yellow solid. ¹H NMR (400 MHz, CHLOROFORM-d) δ 8.81 (d, J=5.8 Hz, 2H), 8.06-8.04 (m, 2H), 7.74-7.70 (m, 2H), 7.41 (t, J=8.1 Hz, 1H), 7.04-6.97 (m, 1H), 4.49 (q, J=7.2 Hz, 2H), 4.44 (bs, 4H), 4.01 (s, 2H), 3.93-3.89 (m, 4H), 3.61-3.56 (m, 2H), 3.56-3.51 (m, 2H), 3.15-3.01 (m, 3H), 1.50 (t, J=7.2 Hz, 3H). LCMS (ESI) for (C27H30N8O2) [M+H]+: 499.3.

### Synthesis of 4-(9-ethyl-2-(3-methoxy-4-(m-tolyl)-1H-pyrazol-1-yl)-9H-purin-6-yl)morpholine (Compound 461)

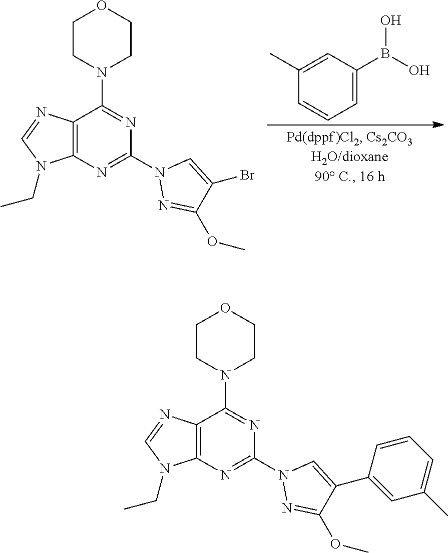

A solution of 4-(2-(4-bromo-3-methoxy-1H-pyrazol-1-yl)-9-ethyl-9H-purin-6-yl)morpholine (80 mg, 0.197 mmol), m-tolylboronic acid (40 mg, 0.296 mmol), 1,1′-bis(diphenylphosphino)ferrocene-palladium(II)dichloride dichloromethane complex (16 mg, 0.02 mmol), and cesium carbonate (160 mg, 0.493 mmol) in 1,4-dioxane/water (5 mL/0.5 mL) was stirred at 90° C. for 16 h under nitrogen atmosphere. The mixture was filtered to remove the solids, the filtrate was concentrated and the residue was subjected to prep-HPLC (BOSTON pHlex ODS 10 μm 21.2×250 mm 120 A. The mobile phase was acetonitrile/0.1% ammonium bicarbonate) to obtain 4-(9-ethyl-2-(3-methoxy-4-(m-tolyl)-1H-pyrazol-1-yl)-9H-purin-6-yl)morpholine (6.2 mg, 7.52%) as white solid. ¹H NMR (400 MHz, DMSO-d₆) δ 8.89 (s, 1H), 8.21 (s, 1H), 7.61 (d, J=9.3 Hz, 2H), 7.28 (t, J=7.5 Hz, 1H), 7.06 (d, J=7.4 Hz, 1H), 4.35 (bs, 4H), 4.23 (q, J=7.2 Hz, 6H), 4.06 (s, 3H), 3.77 (s, 4H), 2.35 (s, 3H), 1.45 (t, J=7.3 Hz, 3H). LCMS (ESI) m/z: 420.1 [M+H]⁺.

### Synthesis of 3-(1-(9-ethyl-6-morpholino-9H-purin-2-yl)-3-methoxy-1H-pyrazol-4-yl)benzonitrile (Compound 462)

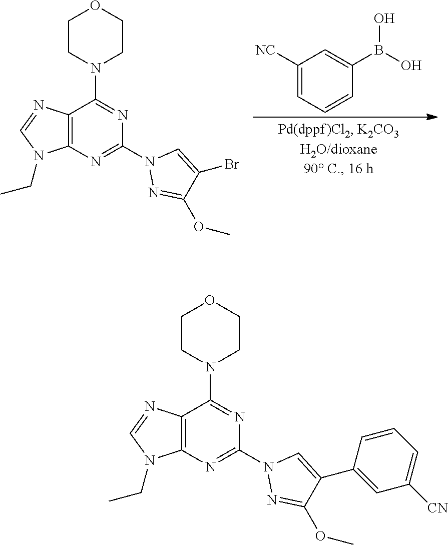 [SMILES: CCn1cnc2c(N3CCOCC3)nc(-n3cc(Br)c(OC)n3)nc21 ; N#Cc1cccc(B(O)O)c1 ; CCn1cnc2c(N3CCOCC3)nc(-n3cc(-c4cccc(C#N)c4)c(OC)n3)nc21]

The compound 124 was synthesized according to the protocol described for compound 123. ¹H NMR (400 MHz, DMSO-d₆) δ 9.13 (s, 1H), 8.28 (s, 1H), 8.23 (s, 1H), 8.18 (d, J=8.0 Hz, 1H), 7.70 (d, J=7.8 Hz, 1H), 7.61 (t, J=7.8 Hz, 1H), 4.35 (bs, 4H), 4.23 (q, J=7.2 Hz, 2H), 4.10 (s, 3H), 3.78 (d, J=4.7 Hz, 4H), 1.46 (t, J=7.3 Hz, 3H). LCMS (ESI) m/z: 431.1 [M+H]⁺.

### Synthesis of (S)-4-(9-ethyl-2-(3-methoxy-4-phenyl-1H-pyrazol-1-yl)-9H-purin-6-yl)-3-methylmorpholine (Compound 463)

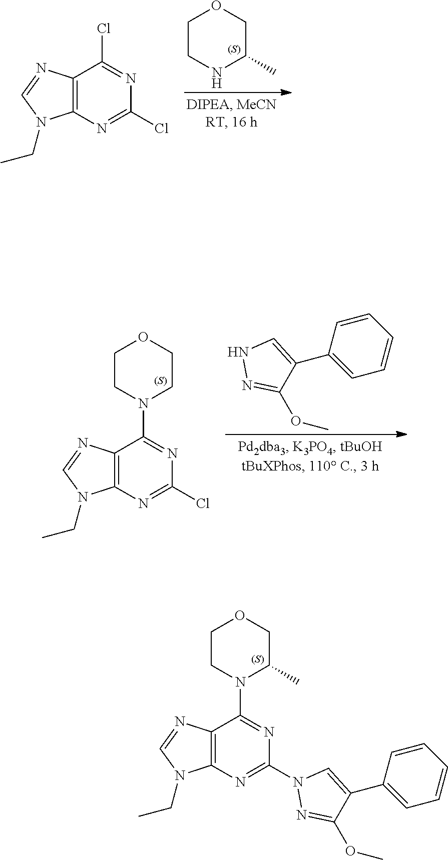

### Step 1: Synthesis of (S)-4-(2-chloro-9-ethyl-9H-purin-6-yl)-3-methylmorpholine

A mixture of 2,6-dichloro-9-ethyl-9H-purine (2.17 g, 10 mmol), N,N-diisopropylethylamine (3.8 g, 30 mmol) and (S)-3-methylmorpholine (2.5 g, 25 mmol) in acetonitrile (30 mL) was stirred at room temperature for 16 h. The resultant precipitate was collected by filtration, washed with acetonitrile to afford (S)-4-(2-chloro-9-ethyl-9H-purin-6-yl)-3-methylmorpholine (2.2 g, 78%) as white solid. LCMS (ESI) m/z: 282.1 [M+H]⁺.

### Step 2: Synthesis of (S)-4-(9-ethyl-2-(3-methoxy-4-phenyl-1H-pyrazol-1-yl)-9H-purin-6-yl)-3-methylmorpholine

A mixture of (S)-4-(2-chloro-9-ethyl-9H-purin-6-yl)-3-methylmorpholine (300 mg, 1.06 mmol), 3-methoxy-4-phenyl-1H-pyrazole (278 mg, 1.6 mmol), potassium phosphate tribasic (674 mg, 3.18 mmol), tris(dibenzylideneacetone)dipalladium (91 mg, 0.1 mmol) and 2-di-tert-butylphosphino-2′,4′,6′-trisopropylbinphenyl (84 mg, 0.2 mmol) in tert-butanol (5 mL) was stirred at 110° C. under argon atmosphere for 3 h. The mixture was filtered to remove the solids and the filtrate was concentrated. The residue was subjected to flash chromatography on silica gel (dichloromethane/methanol 20:1→10:1) to obtain (S)-4-(9-ethyl-2-(3-methoxy-4-phenyl-1H-pyrazol-1-yl)-9H-purin-6-yl)-3-methylmorpholine (303.8 mg, 68%) as white solid. ¹H NMR (500 MHz, DMSO-d₆) δ 8.90 (s, 1H), 8.21 (s, 1H), 7.80 (d, J=7.3 Hz, 2H), 7.40 (t, J=7.7 Hz, 2H), 7.25 (t, J=7.4 Hz, 1H), 5.57 (bs, 1H) 5.06 (bs, 1H), 4.23 (q, J=7.3 Hz, 2H), 4.07 (s, 3H), 4.01 (d, J=8.2 Hz, 1H), 3.79 (d, J=11.4 Hz, 1H), 3.72 (dd, J=11.5, 2.9 Hz, 1H), 3.56 (td, J=11.8, 2.6 Hz, 1H), 3.39 (s, 1H), 1.46 (t, J=7.3 Hz, 3H), 1.35 (d, J=6.7 Hz, 3H). LCMS (ESI) m/z: 420.1 [M+H]⁺.

### Synthesis of Compounds 464-473

Compounds 464-473 were prepared according to methods known to those of skill in the art.

| | | | Comp |
|---|---|---|---|
| Name | Structure | NMR, MS | # |
| 4-{1-phenyl-6- [(pyridin-3- yl)methoxy]-1H- pyrazolo[3,4- d]pyrimidin-4- yl}morpholine | 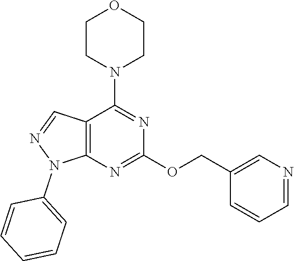 [SMILES: c1ccc(-n2ncc3c(N4CCOCC4)nc(OCc4cccnc4)nc32)cc1] | 1H NMR (400 MHz, DMSO-d6) δ 8.70 (s, 1H), 8.54 (dd, J = 4.8 Hz, 1H), 8.47 (s, 1H), 8.11 (dd, J = 4.8 Hz, 2H), 7.87-7.90 (m, 1H), 7.52-7.56 (m, 2H), 7.32-7.43 (m, 2H), 5.44 (s, 2H), 3.74-3.93 (m, 8H); LCMS (ESI) m/z: 389.2 [M + H]+. | 464 |
| 4-{6-[(4- methanesulfonyl- piperazin-1-yl) methyl]-4- (morpholin-4- yl)thieno[3,2- d]pyrimidin-2-yl}-1H- indazole | 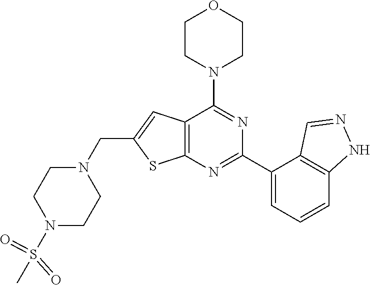 [SMILES: CS(=O)(=O)N1CCN(Cc2cc3c(N4CCOCC4)nc(-c4cccc5[nH]ncc45)nc3s2)CC1] | 1H NMR (400 MHz, DMSO-d6) δ 13.23 (s, 1H), 8.89 (s, 1H), 8.36 (s, 1H), 8.23 (d, J = 7.2 Hz, 1H), 7.67 (d, J = 8.4 Hz, 1H), 7.48 (q, J = 7.2 Hz, 2H), 3.99-4.02 (m, 4H), 3.95 (s, 2H),3.83-3.85 (m, 4H), 3.17 (s, 4H), 2.91 (s, 3H), 2.61 (s, 4H); LCMS (ESI) m/z: 514.2 [M + H]+. | 465 |
| 4-{2-[(2E)-2-[(3- methylphenyl) methylidene] hydrazin-1-yl]-6- (pyridin-4- yl)thieno[2,3- d]pyrimidin-4- yl}morpholine | 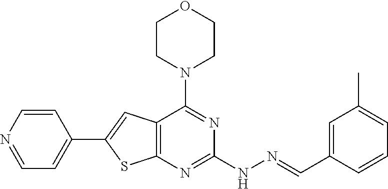 [SMILES: Cc1cccc(/C=N/Nc2nc(N3CCOCC3)c3cc(-c4ccncc4)sc3n2)c1] | 1H NMR (400 MHz, DMSO) δ 11.07 (s, 1H), 8.59 (d, J = 6.0 Hz, 2H), 8.16 (s, 1H), 8.10 (s, 1H), 7.75 (d, J = 6.1 Hz, 2H), 7.47 (d, J = 12.0 Hz, 2H), 7.31-7.29 (m, 1H), 7.18 (d, J = 7.3 Hz, 1H), 3.93 (d, J = 4.7 Hz, 4H), 3.79 (d, J = 4.6 Hz, 4H), 2.36 (s, 3H). LCMS (ESI) m/z: 431.1 [M + H]+. | 466 |
| 2-[(2E)-2-[(3- methylphenyl) methylidene] hydrazin-1-yl]-6- (morpholin-4-yl)-8- (pyridin-4-yl)-7H- purine | 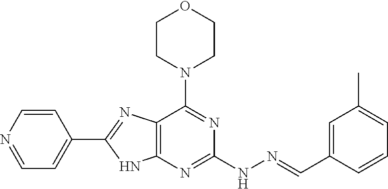 [SMILES: Cc1cccc(/C=N/Nc2nc(N3CCOCC3)c3nc(-c4ccncc4)[nH]c3n2)c1] | 1H NMR (400 MHz, DMSO) δ 13.63 (s, 1H), 10.76 (s, 1H), 8.68 (d, J = 5.4 Hz, 2H), 8.07 (s, 1H), 7.97 (d, J = 5.7 Hz, 2H), 7.56-7.39 (m, 2H), 7.32-7.28 (m, 1H), 7.15 (d, J = 7.3 Hz, 1H), 4.29-4.24 (m, 4H), 3.78-3.75 (m, 4H), 2.35 (s, 3H). LCMS (ESI) m/z: 415.2 [M + H]+. | 467 |
| 4-{5-[(2E)-2-[(3- methylphenyl) methylidene] hydrazin-1-yl]-2- (pyridin-4-yl)- [1,3]oxazolo[5,4- d]pyrimidin-7- yl}morpholine | 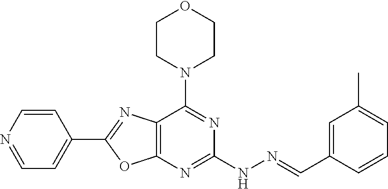 [SMILES: Cc1cccc(/C=N/Nc2nc(N3CCOCC3)c3nc(-c4ccncc4)oc3n2)c1] | 1H NMR (400 MHz, DMSO-d6) δ 11.18 (s, 1H), 8.78 (dd, J = 4.4 Hz, 1.6 Hz, 2H), 8.10(s, 1H), 7.95 (dd, J = 4.4 Hz, 1.6 Hz, 2H), 7.53-7.44 (m, 2H), 7.32 (t, J = 7.6 Hz, 1H), 7.19 (d, J = 7.6 Hz, 1H), 4.18 (brs, 4H), 3.84-3.73 (m, 4H), 2.36 (s, 3H). LCMS (ESI) m/z: 416.1 [M + H]+. | 468 |
| 4-{5-[(2E)-2-[(3- methylphenyl) methylidene] hydrazin-1-yl]-2- (pyridin-4-yl)- [1,3]thiazolo[5,4- d]pyrimidin-7- yl}morpholine | 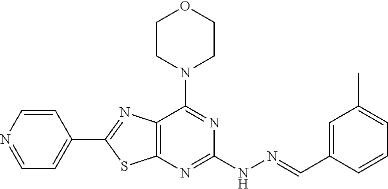 [SMILES: Cc1cccc(/C=N/Nc2nc(N3CCOCC3)c3nc(-c4ccncc4)sc3n2)c1] | 1H NMR (400 MHz, DMSO-d6)δ 11.61 (s, 1H), 8.83 (d, J = 6.4 Hz, 2H), 8.21-8.04 (m, 3H), 7.55 (d, J = 8.4 Hz, 2H), 7.33 (t, J = 7.5 Hz, 1H), 7.22 (d, J = 7.6 Hz, 1H), 4.39 (s, 4H), 3.89-3.75 (m, 4H), 2.36 (s, 3H); LCMS (ESI) m/z: 432.1 [M + H]+. | 469 |
| 4-[2-(2- phenylpyrimidin-4-yl)- 6-(pyridin-4- yl)thieno[2,3- d]pyrimidin-4- yl]morpholine | 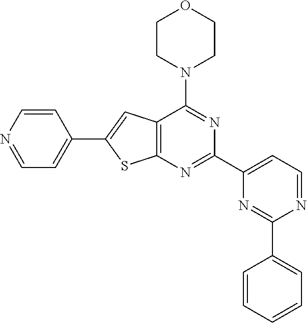 [SMILES: c1ccc(-c2nccc(-c3nc(N4CCOCC4)c4cc(-c5ccncc5)sc4n3)n2)cc1] | 1H NMR (400 MHz, DMSO) δ 9.09 (d, J = 5.1 Hz, 1H), 8.69 (d, J = 6.0 Hz, 2H), 8.54 (d, J = 7.8 Hz, 2H), 8.40 (s, 1H), 8.32 (d, J = 5.0 Hz, 1H), 7.91 (d, J = 6.0 Hz, 2H), 7.59 (d, J = 7.0 Hz, 3H), 4.16-4.09 (m, 4H), 3.89-3.82 (m, 4H). LCMS (ESI) m/z: 453.1 [M + H]+. | 470 |
| 4-[5-(2- phenylpyrimidin-4-yl)- 2-(pyridin-4-yl)- [1,3]thiazolo[5,4- d]pyrimidin-7- yl]morpholine | 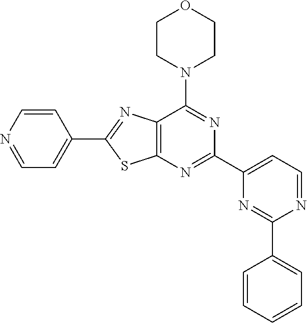 [SMILES: c1ccc(-c2nccc(-c3nc(N4CCOCC4)c4nc(-c5ccncc5)sc4n3)n2)cc1] | 1H NMR (400 MHz, CDCl3) δ 9.00 (d, J = 5.0 Hz, 1H), 8.80 (dd, J = 4.6, 1.4 Hz, 2H), 8.64-8.57 (m, 2H), 8.24 (d, J = 5.0 Hz, 1H), 7.87 (dd, J = 4.4, 1.6 Hz, 2H), 7.57-7.50 (m, 3H), 4.59 (s, 4H), 4.03- 3.93 (m, 4H); LCMS (ESI) m/z: 454.2 [M + H]+. | 471 |
| 4-[2-(1-phenyl-1H- pyrazol-3-yl)-6- (pyridin-4- yl)thieno[2,3- d]pyrimidin-4- yl]morpholine | 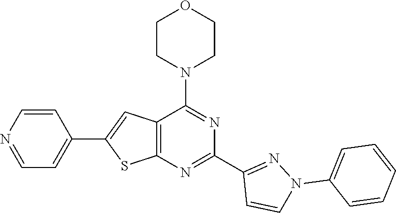 [SMILES: c1ccc(-n2ccc(-c3nc(N4CCOCC4)c4cc(-c5ccncc5)sc4n3)n2)cc1] | 1H NMR (400 MHz, DMSO) δ 8.66 (d, J = 6.0 Hz, 2H), 8.61 (d, J = 2.5 Hz, 1H), 8.32 (s, 1H), 7.96 (d, J = 8.0 Hz, 2H), 7.86 (d, J = 6.1 Hz, 2H), 7.56 (t, J = 7.9 Hz, 2H), 7.37 (t, J = 7.4 Hz, 1H), 7.21 (d, J = 2.4 Hz, 1H), 4.12-4.01 (m, 4H), 3.89-3.79 (m, 4H). LCMS (ESI) m/z: 441.1 [M + H]+. | 472 |
| 4-[5-(2- phenylpyrimidin-4-yl)- 2-(pyridin-4-yl)- [1,3]thiazolo[4,5- d]pyrimidin-7- yl]morpholine | 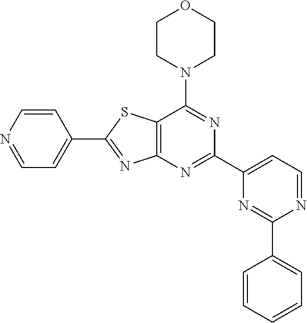 [SMILES: c1ccc(-c2nccc(-c3nc(N4CCOCC4)c4sc(-c5ccncc5)nc4n3)n2)cc1] | 1H NMR (400 MHz, DMSO-d6)δ 9.12 (d, J = 5.2 Hz, 1H), 8.88 (dd, J = 4.4, 1.6 Hz, 2H), 8.56 (dd, J = 7.2, 2.6 Hz, 2H), 8.36 (d, J = 5.2 Hz, 1H), 8.15 (dd, J = 4.4, 1.6 Hz, 2H), 7.68-7.52 (m, 3H), 4.17-4.06 (m, 4H), 3.95-3.77 (m, 4H); LCMS (ESI) m/z: 454.1 [M + H]+. | 473 |

### General Procedure 1

### Preparation of 4-(9-ethyl-2-(3-phenyl-H-pyrazol-1-yl)-8-(pyridin-4-yl)-9H-purin-6-yl)morpholine Compound 158

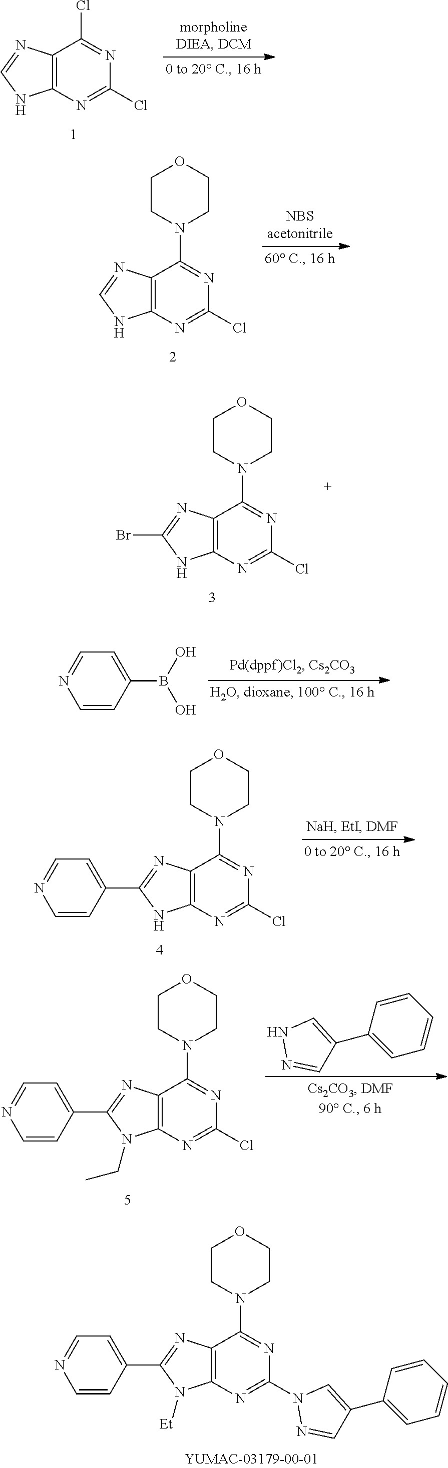

### Step 1: Preparation of 4-(2-chloro-9H-purin-6-yl)morpholine

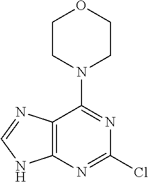 [SMILES: Clc1nc(N2CCOCC2)c2nc[nH]c2n1]

A solution of 2,6-dichloro-9H-purine (2.0 g, 10.64 mmol) in dichloromethane (50 mL) was added morpholine (2.78 g, 31.92 mmol) and diisopropylethylamine (4.13 g, 31.92 mmol). Then the residue was stirred at 20° C. for 16 hour. The reaction was successful and the residue was concentrated under reduced pressure, slurried in ethanol, water and filtered to offer 4-(2-chloro-9H-purin-6-yl)morpholine (2.3 g, crude) as a white solid.

LCMS (ESI) m/z: 240.1 [M+H]+.

### Step 2: Preparation of 4-(8-bromo-2-chloro-9H-purin-6-yl)morpholine

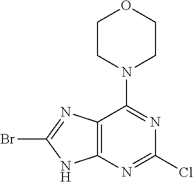 [SMILES: Clc1nc(N2CCOCC2)c2nc(Br)[nH]c2n1]

A solution of 4-(2-chloro-9H-purin-6-yl)morpholine (2.2 g, 9.20 mmol) in acetonitrile (100 mL) was added bromosuccinimide (2.95 g, 16.56 mmol) heated to 60° C. and stirred at 60° C. for 16 hour. The reaction was successful and the residue was filtered to offer 4-(8-bromo-2-chloro-9H-purin-6-yl)morpholine (2.5 g, crude) as a white solid.

LCMS (ESI) m/z: 318.0 [M+H]+.

### Step 3: Preparation of 4-(2-chloro-8-(pyridin-4-yl)-9H-purin-6-yl)morpholine

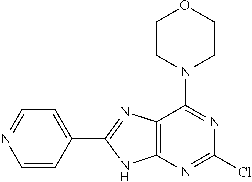 [SMILES: Clc1nc(N2CCOCC2)c2nc(-c3ccncc3)[nH]c2n1]

A solution of 4-(8-bromo-2-chloro-9H-purin-6-yl)morpholine (1.0 g, 3.157 mmol), pyridin-4-ylboronic acid (621 mg, 5.051 mmol), cesium carbonate (3.09 g, 9.470 mmol), water (20 mL) in dioxane (80 mL) was added [1,1′-Bis(diphenylphosphino)ferrocene]palladium(II) Dichloride Dichloromethane Adduct (258 mg, 0.316 mmol). Then the residue was heated to 100° C. and stirred for 16 hour. The reaction was successful and further purified by silica gel column (dichloromethane:/methanol=20:1) to offer 4-(2-chloro-8-(pyridin-4-yl)-9H-purin-6-yl)morpholine (430 mg, crude) as a light yellow solid.

LCMS (ESI) m/z: 317.1 [M+H]+.

### Step 4: Preparation of 4-(2-chloro-9-ethyl-8-(pyridin-4-yl)-9H-purin-6-yl)morpholine

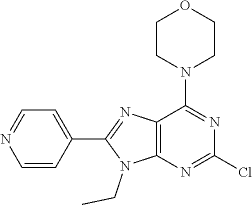 [SMILES: CCn1c(-c2ccncc2)nc2c(N3CCOCC3)nc(Cl)nc21]

A solution of 4-(2-chloro-8-(pyridin-4-yl)-9H-purin-6-yl)morpholine (420 mg, 1.329 mmol) in N,N-Dimethylformamide (5 mL) was added sodium hydrogen (106 mg, 2.657 mmol) at 0° C. Then the residue was stirred at 20° C. for 2 hour. A solution of ethyl iodide (414 mg, 2.657 mmol) in N,N-Dimethylformamide (1 mL) was added to the above solution. Then the reaction was stirred for 16 hour. The reaction was successful, the residue was purified by silica gel column (dichloromethane:/methanol=20:1) to offer 4-(2-chloro-9-ethyl-8-(pyridin-4-yl)-9H-purin-6-yl)morpholine (160 mg, crude) as a light yellow solid.

LCMS (ESI) m/z: 345.1 [M+H]+.

### Step 5: Preparation of 4-(9-ethyl-2-(3-phenyl-1H-pyrazol-1-yl)-8-(pyridin-4-yl)-9H-purin-6-yl)morpholine

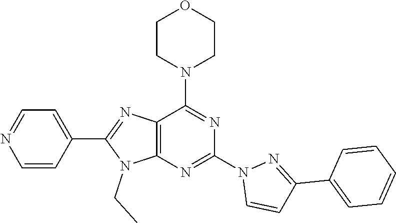 [SMILES: CCn1c(-c2ccncc2)nc2c(N3CCOCC3)nc(-n3ccc(-c4ccccc4)n3)nc21]

A solution of 4-(2-chloro-9-ethyl-8-(pyridin-4-yl)-9H-purin-6-yl)morpholine (80 mg, 0.232 mmol), 3-phenyl-1H-pyrazole (67 mg, 0.465 mmol) in N,N-Dimethylformamide (6 mL) was added cesium carbonate (227 mg, 0.696 mmol). Then the residue was heated to 90° C. and stirred for 6 hour. The reaction was successful and further purified by HPLC (SunFire C18, 4.6*50 mm, 3.5 um column Xbridge C18 3.5 μm 4.6×50 mm column. The elution system used was a gradient of 5%-95% over 1.5 min at 2 ml/min and the solvent was acetonitrile/0.01% aqueous HCOOH) to offer 4-(9-ethyl-2-(3-phenyl-1H-pyrazol-1-yl)-8-(pyridin-4-yl)-9H-purin-6-yl)morpholine (23 mg, 5.1%) as a white solid.

¹H NMR (400 MHz, DMSO) δ 8.79 (dd, J=11.1, 4.3 Hz, 3H), 8.08-7.95 (m, 2H), 7.86 (dd, J=4.5, 1.6 Hz, 2H), 7.48 (t, J=7.5 Hz, 2H), 7.40 (d, J=7.3 Hz, 1H), 7.06 (d, J=2.7 Hz, 1H), 4.46 (m, 6H), 3.86-3.59 (m, 4H), 1.36 (t, J=7.2 Hz, 3H).

LCMS (ESI) m/z: 453.2 [M+H]+.

### General Procedure 2 (Compounds 1, 94, 96, 158-163, 165-169, 171-177, 179, 180, 195, and 299)

### Preparation of 9-methyl-6-(morpholin-4-yl)-2-[3-(pyridin-3-yl)-1H-pyrazol-1-yl]-8-(pyridin-4-yl)-9H-purine

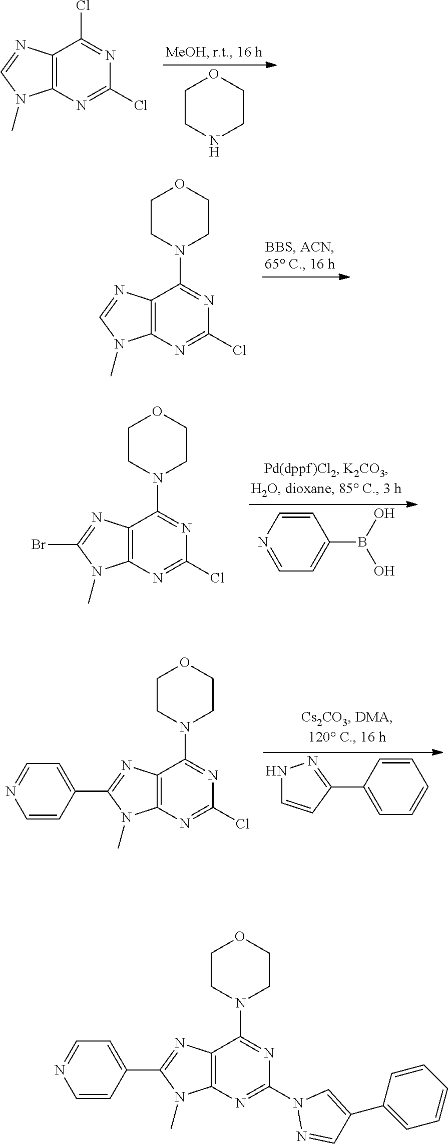

### Step 1: Preparation of 4-(2-Chloro-9-methyl-9H-purin-6-yl)morpholine

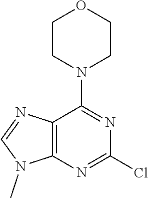 [SMILES: Cn1cnc2c(N3CCOCC3)nc(Cl)nc21]

A mixture of 2,6-dichloro-9-methyl-9H-purine (6.00 g, 30 mmol) and morpholine (6.50 g, 74 mmol) in methanol (300 mL) was stirred at room temperature for 16 hours. The mixture was filtered directly, and the residue was triturated with methanol. Product 4-(2-Chloro-9-methyl-9H-purin-6-yl)morpholine (7.00 g, 28 mmol, 93%) was afforded as a white solid and carried onto next step without further purification. NMR data unavailable; LCMS (ESI) m/z: 254.1 [M+H]⁺.

### Step 2: Preparation of 4-(8-Bromo-2-chloro-9-methyl-9H-purin-6-yl)morpholine

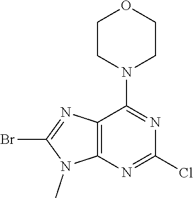 [SMILES: Cn1c(Br)nc2c(N3CCOCC3)nc(Cl)nc21]

A mixture of 4-(2-chloro-9-methyl-9H-purin-6-yl)morpholine (7.00 g, 28 mmol) and N-bromosuccinimide (8.80 g, 50 mmol) in acetonitrile (500 mL) was stirred at 65° C. for 16 hours. The mixture was filtered directly, and the residue was triturated with acetonitrile. Product 4-(8-Bromo-2-chloro-9-methyl-9H-purin-6-yl)morpholine (8.00 g, 24 mmol, 87%) was afforded as light yellow solid and carried onto next step without further purification. NMR data unavailable; LCMS (ESI) m/z: 332.3 [M+H]⁺.

### Step 3: Preparation of 4-(2-Chloro-9-methyl-8-(pyridin-4-yl)-9H-purin-6-yl)morpholine

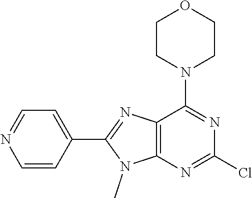 [SMILES: Cn1c(-c2ccncc2)nc2c(N3CCOCC3)nc(Cl)nc21]

In a reaction vial, 4-(8-bromo-2-chloro-9-methyl-9H-purin-6-yl)morpholine (5.00 g, 15 mmol), pyridin-4-ylboronic acid (2.20 g, 18 mmol), 1,1′-bis(diphenylphosphino)ferrocene-palladium(II)dichloride (1.10 g, 1.5 mmol) and potassium carbonate (5.20 g, 38 mmol) were suspended in dioxane (50 mL) and water (5 mL) under nitrogen. The reaction mixture was stirred at 85° C. for 3 hours. The reaction was filtered over celite and washed with ethyl acetate (3×25 mL). The filtrate was concentrated under reduced pressure. Crude product was purified via flash column chromatography through silica gel using a gradient of 0-5% methanol in dichloromethane. Product 4-(2-Chloro-9-methyl-8-(pyridin-4-yl)-9H-purin-6-yl)morpholine (3.00 g, 9.1 mmol, 60%) was afforded as a light yellow solid. NMR data unavailable; LCMS (ESI) m/z: 331.1 [M+H]⁺.

### Step 4: Preparation of 4-(9-methyl-2-(3-phenyl-1H-pyrazol-1-yl)-8-(pyridin-4-yl)-9H-purin-6-yl)morpholine

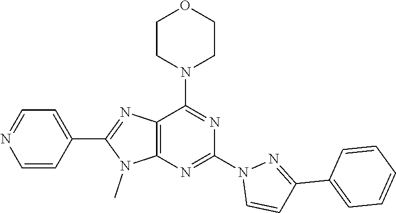 [SMILES: Cn1c(-c2ccncc2)nc2c(N3CCOCC3)nc(-n3ccc(-c4ccccc4)n3)nc21]

A mixture of 4-(2-chloro-9-methyl-8-(pyridin-4-yl)-9H-purin-6-yl)morpholine (100 mg, 0.30 mmol), 3-phenyl-1H-pyrazole (58.0 mg, 0.40 mmol) and cesium carbonate (196 mg, 0.60 mmol) in N,N-dimethylacetamide (5 mL) was stirred at 120° C. for 16 hours. The product was indicated present via UPLC analysis. The mixture was allowed to cool to room temperature, quenched with water (10 mL) and the organics were extracted with ethyl acetate (3×10 mL). The organic layers were pooled, washed with brine (10 mL), dried over sodium sulfate, filtered, and concentrated under reduced pressure. Crude product was purified by prep-HPLC (the crude samples were dissolved in methanol unless otherwise noted before purification. Boston C18 21*250 mm 10 μm column. The mobile phase was acetonitrile/0.01% aqueous ammonium bicarbonate). Product 4-(9-methyl-2-(3-phenyl-1H-pyrazol-1-yl)-8-(pyridin-4-yl)-9H-purin-6-yl)morpholine (25.6 mg, 0.058 mmol, 19%) was afforded as a white solid. ¹H NMR (400 MHz, Dimethylsulfoxide-d₆) δ 9.17 (d, J=2.3 Hz, 1H), 8.82 (d, J=2.7 Hz, 1H), 8.80-8.76 (m, 2H), 8.59 (dd, J=4.8, 1.6 Hz, 1H), 8.33 (dt, J=7.9, 1.9 Hz, 1H), 7.94-7.87 (m, 2H), 7.51 (dd, J=7.9, 4.8 Hz, 1H), 7.17 (d, J=2.5 Hz, 1H), 4.23 (s, 4H), 3.97 (s, 3H), 3.80 (t, J=4.8 Hz, 4H); LCMS (ESI) m/z: 439 [M+H]⁺.

Compounds

| # | Structure | LCMS Data | ¹H NMR Data |
|---|---|---|---|
| 94 | 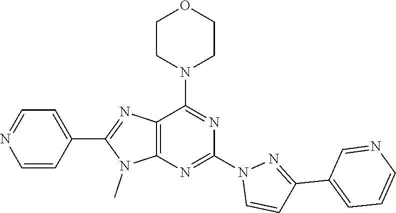 [SMILES: Cn1c(-c2ccncc2)nc2c(N3CCOCC3)nc(-n3ccc(-c4cccnc4)n3)nc21] | LCMS (ESI) m/z: 439 [M + H]⁺. | ¹H NMR (400 MHz, Dimethylsulfoxide-d₆) δ 9.17 (d, J = 2.3 Hz, 1H), 8.82 (d, J = 2.7 Hz, 1H), 8.80-8.76 (m, 2H), 8.59 (dd, J = 4.8, 1.6 Hz, 1H), 8.33 (dt, J = 7.9, 1.9 Hz, 1H), 7.94-7.87 (m, 2H), 7.51 (dd, J = 7.9, 4.8 Hz, 1H), 7.17 (d, J = 2.5 Hz, 1H), 4.23 (s, 4H), 3.97 (s, 3H), 3.80 (t, J = 4.8 Hz, 4H). |
| 163 | 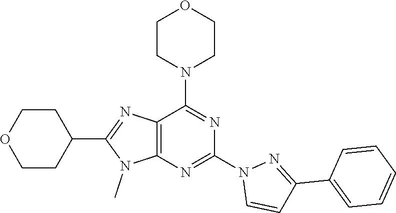 [SMILES: Cn1c(C2CCOCC2)nc2c(N3CCOCC3)nc(-n3ccc(-c4ccccc4)n3)nc21] | LCMS (ESI) m/z: 446 [M + H]⁺. | ¹H NMR (400 MHz, Dimethylsulfoxide-d₆) δ 8.72 (d, J = 2.7 Hz, 1H), 7.99-7.93 (m, 2H), 7.47 (dd, J = 8.3, 7.0 Hz, 2H), 7.41-7.35 (m, 1H), 7.02 (d, J = 2.7 Hz, 1H), 4.30 (s, 4H), 3.99- 3.94 (m, 2H), 3.80-3.75 (m, 7H), 3.51 (td, J = 11.3, 3.2 Hz, 2H), 3.31-3.25 (m, 1H), 1.92- 1.77 (m, 4H). |
| 195 | 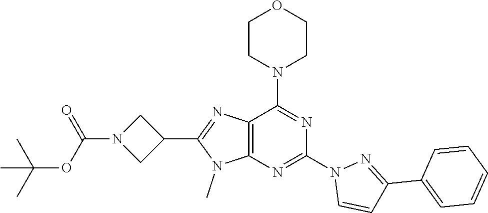 [SMILES: Cn1c(C2CN(C(=O)OC(C)(C)C)C2)nc2c(N3CCOCC3)nc(-n3ccc(-c4ccccc4)n3)nc21] | LCMS (ESI) m/z: 517.2 [M + H]⁺. | ¹H NMR (400 MHz, Dimethylsulfoxide-d₆) δ 8.73 (d, J = 2.6 Hz, 1H), 7.96 (d, J = 7.2 Hz, 2H), 7.47 (t, J = 7.5 Hz, 2H), 7.38 (t, J = 7.3 Hz, 1H), 7.04 (d, J = 2.7 Hz, 1H), 4.84-4.05 (m, 9H), 3.92-3.72 (m, 4H), 3.64 (s, 3H), 1.40 (s, 9H). |
| 165 | 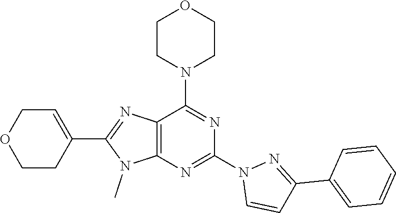 [SMILES: Cn1c(C2=CCOCC2)nc2c(N3CCOCC3)nc(-n3ccc(-c4ccccc4)n3)nc21] | LCMS (ESI) m/z: 444 [M + H]⁺. | ¹H NMR (400 MHz, Dimethylsulfoxide-d₆) δ 8.72 (d, J = 2.7 Hz, 1H), 7.99-7.90 (m, 2H), 7.47 (t, J = 7.6 Hz, 2H), 7.38 (t, J = 7.4 Hz, 1H), 7.02 (d, J = 2.6 Hz, 1H), 6.57 (dd, J = 6.2, 2.1 Hz, 1H), 4.88 (dd, J = 6.3, 3.3 Hz, 1H), 4.43-4.17 (m, 4H), 4.09- 4.04 (m, 1H), 3.96-3.91 (m, 1H), 3.82-3.72 (m, 7H), 3.30 (s, 1H), 2.24-2.11 (m, 2H). |
| 174 | 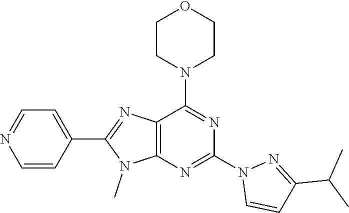 [SMILES: CC(C)c1ccn(-c2nc(N3CCOCC3)c3nc(-c4ccncc4)n(C)c3n2)n1] | LCMS (ESI) m/z: 405 [M + H]⁺. | ¹H NMR (400 MHz, Dimethylsulfoxide-d₆) δ 8.79-8.77 (m, 2H), 8.59 (d, J = 2.8 Hz, 1H), 7.91-7.90 (m, 2H), 6.41 (d, J = 2.8 Hz, 1H), 4.39-4.22 (m, 4H), 3.93 (s, 1H), 3.79-3.77 (m, 4H), 3.06-3.00 (m, 1H), 1.27-1.26 (m, 6H). |

### Example 2. PIKfyve Inhibitory Activity

PIKfyve Biochemical Assay. The biochemical PIKFyve inhibition assays were run by Carna Biosciences according to proprietary methodology based on the Promega ADP-Glo™ Kinase assay. A full-length human PIKFYVE [1-2098(end) amino acids and S696N, L932S, Q995L, T998S, S1033 A and Q1183K of the protein having the sequence set forth in NCBI Reference Sequence No. NP_055855.2] was expressed as N-terminal GST-fusion protein (265 kDa) using baculovirus expression system. GST-PIKFYVE was purified by using glutathione sepharose chromatography and used in an ADP-Glo™ Kinase assay (Promega). Reactions were set up by adding the test compound solution, substrate solution, ATP solution and kinase solution, each at 4× final concentrations. Reactions were prepared with assay buffer (50 mM MOPS, 1 mM DTT, pH7.2), mixed, and incubated in black 384 well polystyrene plates for 1 hour at room temperature. ADP-Glo™ reagent was then added for 40 minutes, followed by kinase detection reagent for an additional 40 minutes. The kinase activity was evaluated by detecting relative light units on a luminescence plate reader. Samples were run in duplicate from 10 μM to 3 nM. Data was analyzed by setting the control wells (+PIKfyve, no compound) to 0% inhibition and the readout value of background (no PIKfyve) set to 100% inhibition, then the % inhibition of each test solution calculated. IC50 values were calculated from concentration vs % inhibition curves by fitting to a four-parameter logistic curve.

NanoBRE™ TE Intracellular Kinase Assay, K-8 (Promega) Cell-Based Assay. Intracellular inhibition of PIKfyve was assayed using Promega's NanoBRET™ TE Intracellular Kinase Assay, K-8 according to manufacturer's instructions. A dilution series of test compounds was added for 2 hours to HEK293 cells transfected for a minimum of 20 hours with PIKFYVE-NanoLuc@Fusion Vector (Promega) containing a full-length PIKfyve according to manufacturer's specifications in a 96-well plate. Kinase activity was detected by addition of a NanoBRET™ tracer reagent, which was a proprietary PIKfyve inhibitor appended to a fluorescent probe (BRET, bioluminescence resonance energy transfer). Test compounds were tested at concentrations of 10, 3, 1, 0.3, 0.1, 0.03, 0.01, 0.003 μM. BRET signals were measured by a GloMax®Discover Multimode Microplate Reader (Promega) using 0.3 sec/well integration time, 450BP donor filter and 600LP acceptor filters. Active test compounds that bound PIKfyve and displaced the tracer reduced BRET signal. IC50 values were then calculated by fitting the data to the normalized BRET ratio.

The results of the PIKfyve inhibition assays are summarized in the table below.

| | PIKFyve |
|---|---|
| # | IC₅₀ (μM) |
| 1 | ++ |
| 2 | − |
| 3 | +++ |
| 4 | +++ |
| 5 | ++++ |
| 6 | ++++ |
| 7 | ++++ |
| 8 | +++ |
| 9 | +++ |
| 10 | ++++ |
| 11 | +++ |
| 12 | ++ |
| 13 | ++++ |
| 14 | +++ |
| 15 | +++ |
| 16 | +++ |
| 17 | ++ |
| 18 | ++ |
| 19 | ++ |
| 20 | +++ |
| 21 | ++ |
| 22 | ++++ |
| 23 | ++++ |
| 24 | ++++ |
| 25 | |
| 26 | |
| 27 | ++++ |
| 28 | ++++ |
| 29 | ++++ |
| 30 | ++++ |
| 31 | ++++ |
| 32 | |
| 33 | ++++ |
| 34 | ++++ |
| 35 | |
| 36 | ++++ |
| 37 | |
| 38 | |
| 39 | |
| 40 | |
| 41 | ++++ |
| 42 | ++++ |
| 43 | +++ |
| 44 | +++ |
| 45 | ++ |
| 46 | +++ |
| 47 | |
| 48 | +++ |
| 49 | +++ |
| 50 | ++ |
| 51 | +++ |
| 52 | +++ |
| 53 | +++ |
| 54 | ++++ |
| 55 | |
| 56 | − |
| 57 | ++++ |
| 58 | ++ |
| 59 | +++ |
| 60 | +++ |
| 61 | ++ |
| 62 | ++++ |
| 63 | |
| 64 | ++ |
| 65 | +++ |
| 66 | ++ |
| 67 | +++ |
| 68 | ++++ |
| 69 | |
| 70 | |
| 71 | ++++ |
| 72 | |
| 73 | |
| 74 | |
| 75 | |
| 76 | +++ |
| 77 | +++ |
| 78 | ++ |
| 79 | ++ |
| 80 | ++ |
| 81 | ++ |
| 82 | +++ |
| 83 | +++ |
| 84 | +++ |
| 85 | +++ |
| 86 | ++++ |
| 87 | |
| 88 | ++++ |
| 89 | ++ |
| 90 | +++ |
| 91 | ++ |
| 92 | +++ |
| 93 | +++ |
| 94 | +++ |
| 95 | ++ |
| 96 | + |
| 97 | +++ |
| 98 | +++ |
| 99 | +++ |
| 100 | ++++ |
| 101 | +++ |
| 102 | +++ |
| 103 | +++ |
| 104 | +++ |
| 105 | + |
| 106 | ++++ |
| 107 | ++++ |
| 108 | ++++ |
| 109 | +++ |
| 110 | ++++ |
| 111 | |
| 112 | |
| 113 | ++++ |
| 114 | ++++ |
| 115 | |
| 116 | ++ |
| 117 | +++ |
| 118 | +++ |
| 119 | ++ |
| 120 | ++ |
| 121 | +++ |
| 122 | +++ |
| 123 | ++++ |
| 124 | |
| 125 | |
| 126 | +++ |
| 127 | ++ |
| 128 | +++ |
| 129 | +++ |
| 130 | ++++ |
| 131 | ++ |
| 132 | ++ |
| 133 | + |
| 134 | ++ |
| 135 | +++ |
| 136 | ++ |
| 137 | ++ |
| 138 | + |
| 139 | ++ |
| 140 | ++++ |
| 141 | ++++ |
| 142 | ++++ |
| 143 | +++ |
| 144 | ++ |
| 145 | ++ |
| 146 | ++ |
| 147 | ++ |
| 148 | ++ |
| 149 | ++ |
| 150 | +++ |
| 151 | ++++ |
| 152 | ++ |
| 153 | +++ |
| 154 | |
| 155 | +++ |
| 156 | +++ |
| 157 | ++ |
| 158 | +++ |
| 159 | +++ |
| 160 | ++ |
| 161 | ++ |
| 162 | ++ |
| 163 | ++ |
| 165 | +++ |
| 166 | − |
| 167 | − |
| 168 | ++ |
| 169 | ++ |
| 171 | − |
| 172 | +++ |
| 173 | +++ |
| 174 | ++ |
| 175 | ++ |
| 176 | ++ |
| 177 | +++ |
| 178 | +++ |
| 179 | +++ |
| 180 | ++ |
| 181 | ++ |
| 182 | ++ |
| 183 | +++ |
| 184 | ++ |
| 185 | ++ |
| 186 | ++ |
| 187 | − |
| 188 | ++++ |
| 189 | ++++ |
| 190 | +++ |
| 191 | ++ |
| 192 | ++ |
| 193 | ++ |
| 194 | ++ |
| 195 | +++ |
| 196 | ++ |
| 197 | ++ |
| 198 | |
| 199 | |
| 200 | |
| 201 | |
| 202 | |
| 203 | |
| 204 | |
| 205 | |
| 206 | |
| 207 | +++ |
| 208 | +++ |
| 209 | ++++ |
| 210 | |
| 211 | |
| 212 | ++++ |
| 213 | ++++ |
| 214 | ++++ |
| 215 | ++++ |
| 216 | ++++ |
| 217 | |
| 218 | |
| 219 | ++++ |
| 220 | +++ |
| 221 | ++ |
| 222 | ++ |
| 223 | ++ |
| 224 | ++ |
| 225 | ++++ |
| 226 | ++ |
| 227 | |
| 228 | ++++ |
| 229 | |
| 230 | |
| 231 | |
| 232 | ++++ |
| 233 | +++ |
| 234 | ++ |
| 235 | ++ |
| 236 | ++++ |
| 237 | |
| 238 | ++++ |
| 239 | |
| 240 | |
| 241 | |
| 242 | +++ |
| 243 | ++++ |
| 244 | ++++ |
| 245 | |
| 246 | |
| 247 | |
| 248 | |
| 249 | ++++ |
| 250 | +++ |
| 251 | ++ |
| 252 | ++++ |
| 253 | ++++ |
| 254 | ++++ |
| 255 | |
| 256 | |
| 257 | |
| 258 | ++++ |
| 259 | |
| 260 | ++++ |
| 261 | +++ |
| 262 | ++ |
| 263 | ++++ |
| 264 | ++++ |
| 265 | ++++ |
| 266 | ++++ |
| 267 | ++++ |
| 268 | |
| 269 | |
| 270 | |
| 271 | |
| 272 | ++++ |
| 273 | ++++ |
| 274 | |
| 275 | ++++ |
| 276 | |
| 277 | |
| 278 | |
| 279 | +++ |
| 280 | +++ |
| 281 | +++ |
| 282 | +++ |
| 283 | +++ |
| 284 | ++ |
| 285 | ++++ |
| 286 | ++ |
| 287 | +++ |
| 288 | + |
| 289 | +++ |
| 290 | + |
| 291 | ++ |
| 292 | ++++ |
| 293 | +++ |
| 294 | ++ |
| 295 | ++ |
| 296 | +++ |
| 297 | ++ |
| 298 | ++ |
| 299 | ++ |
| 300 | ++++ |
| 301 | ++++ |
| 302 | +++ |
| 303 | ++++ |
| 304 | ++++ |
| 305 | ++ |
| 306 | +++ |
| 307 | ++++ |
| 308 | ++++ |
| 309 | ++++ |
| 310 | |
| 311 | ++ |
| 312 | +++ |
| 313 | ++++ |
| 314 | ++++ |
| 315 | |
| 316 | +++ |
| 317 | |
| 318 | |
| 319 | |
| 320 | |
| 321 | |
| 322 | |
| 323 | ++ |
| 324 | ++++ |
| 325 | ++++ |
| 326 | ++++ |
| 327 | +++ |
| 328 | ++++ |
| 329 | ++++ |
| 330 | ++++ |
| 331 | − |
| 332 | ++++ |
| 333 | ++ |
| 334 | ++++ |
| 335 | ++++ |
| 336 | ++++ |
| 337 | |
| 338 | |
| 339 | 4.41 |
| 340 | ++++ |
| 341 | ++++ |
| 342 | ++++ |
| 343 | ++++ |
| 344 | ++++ |
| 345 | ++++ |
| 346 | ++++ |
| 347 | |
| 348 | +++ |
| 349 | |
| 350 | +++ |
| 351 | +++ |
| 352 | ++++ |
| 353 | +++ |
| 354 | ++++ |
| 355 | ++++ |
| 356 | +++ |
| 357 | |
| 358 | + |
| 359 | ++++ |
| 360 | +++ |
| 361 | |
| 362 | +++ |
| 363 | +++ |
| 364 | + |
| 365 | ++++ |
| 366 | ++++ |
| 367 | + |
| 368 | ++++ |
| 369 | |
| 370 | |
| 371 | ++ |
| 372 | |
| 373 | ++++ |
| 374 | ++++ |
| 375 | ++++ |
| 376 | |
| 377 | ++++ |
| 378 | |
| 379 | ++++ |
| 380 | |
| 381 | |
| 382 | |
| 383 | ++++ |
| 384 | |
| 385 | ++ |
| 386 | ++++ |
| 387 | |
| 388 | |
| 389 | |
| 390 | +++ |
| 391 | + |
| 392 | |
| 393 | |
| 394 | |
| 395 | |
| 396 | +++ |
| 397 | ++++ |
| 398 | +++ |
| 399 | ++++ |
| 400 | ++++ |
| 401 | |
| 402 | |
| 403 | ++++ |
| 404 | |
| 405 | |
| 406 | |
| 407 | |
| 408 | |
| 409 | ++++ |
| 410 | ++ |
| 411 | ++++ |
| 412 | |
| 413 | ++++ |
| 414 | |
| 415 | +++ |
| 416 | |
| 417 | |
| 418 | ++++ |
| 419 | +++ |
| 420 | |
| 421 | ++++ |
| 422 | |
| 423 | ++ |
| 424 | ++ |
| 425 | ++++ |
| 426 | |
| 427 | +++ |
| 428 | +++ |
| 429 | |
| 430 | |
| 431 | |
| 432 | + |
| 433 | + |
| 434 | |
| 435 | |
| 436 | |
| 437 | + |
| 438 | |
| 439 | |
| 440 | |
| 441 | |
| 442 | |
| 443 | ++ |
| 444 | ++++ |
| 445 | ++++ |
| 446 | |
| 447 | |
| 448 | |
| 449 | |
| 450 | +++ |
| 451 | |
| 452 | |
| 453 | ++++ |
| 454 | ++++ |
| 455 | |
| 456 | |
| 457 | ++++ |
| 458 | +++ |
| 459 | |
| 460 | |
| 461 | |
| 462 | |
| 463 | |
| 464 | + |
| 465 | |
| 466 | +++ |
| 467 | ++++ |
| 468 | ++++ |
| 469 | ++++ |
| 470 | ++ |
| 471 | +++ |
| 472 | +++ |
| 473 | ++ |
| 474 | |
| 475 | |
| 476 | |

The results of PIKFyve EEA1 assays are shown below.

Endosomal Vesicle Enlargement Assay. Genetic or pharmacological disruption of PIKfyve activity results in enlargement of endosomal vesicles. This enlargement was utilized as a surrogate readout of PIKFyve inhibition for routine triage of PIKfyve inhibitors. U2S cells grown in 96-well assay plates were treated with compound diluted in DMEM media containing 10% fetal bovine serum. After 3 hours of treatment, cells were fixed with paraformaldehyde, permeabilized with 0.2% Triton-X in phosphate buffered saline and stained against EEA1. During the secondary antibody staining, cells were also stained with CellMask DeepRed and Hoechst to detect cytoplasms and nuclei respectively. Endosomal structures were visualized using a high content imager at 40× magnification. Images were analyzed using a linear classifier algorithm integrating EEA1 spot size, intensity and texture trained on images of cells treated with the potent reference compound APY0201. Compound activity was calculated by subtracting the DMSO signal and calculating percentage activity relative to maximal APY0201 activity. IC50s were then calculated from concentration vs. % inhibition data by logistic regression.

| | PIKFyve | | PIKFyve | | PIKFyve | | PIKFyve | | PIKFyve | | PIKFyve |
|---|---|---|---|---|---|---|---|---|---|---|---|
| | EEA1 | | EEA1 | | EEA1 | | EEA1 | | EEA1 | | EEA1 |
| # | IC₅₀ (μM) | # | IC₅₀ (μM) | # | IC₅₀ (μM) | # | IC₅₀ (μM) | # | IC₅₀ (μM) | # | IC₅₀ (μM) |
| 1 | | 58 | + | 115 | + | 172 | + | 229 | + | 286 | |
| 2 | | 59 | +++ | 116 | | 173 | + | 230 | + | 287 | |
| 3 | | 60 | + | 117 | + | 174 | + | 231 | ++ | 288 | |
| 4 | + | 61 | | 118 | + | 175 | | 232 | ++ | 289 | |
| 5 | ++ | 62 | + | 119 | + | 176 | | 233 | + | 290 | + |
| 6 | + | 63 | + | 120 | + | 177 | | 234 | + | 291 | |
| 7 | ++ | 64 | | 121 | + | 178 | | 235 | + | 292 | |
| 8 | + | 65 | + | 122 | + | 179 | | 236 | ++ | 293 | + |
| 9 | + | 66 | + | 123 | ++ | 180 | | 237 | + | 294 | |
| 10 | | 67 | ++ | 124 | + | 181 | + | 238 | ++ | 295 | |
| 11 | + | 68 | + | 125 | + | 182 | + | 239 | + | 296 | |
| 12 | + | 69 | + | 126 | | 183 | + | 240 | + | 297 | |
| 13 | ++ | 70 | + | 127 | + | 184 | | 241 | + | 298 | |
| 14 | | 71 | +++ | 128 | + | 185 | + | 242 | + | 299 | |
| 15 | + | 72 | + | 129 | + | 186 | | 243 | + | 300 | ++ |
| 16 | | 73 | + | 130 | ++ | 187 | | 244 | + | 301 | +++ |
| 17 | | 74 | + | 131 | | 188 | | 245 | + | 302 | + |
| 18 | | 75 | + | 132 | | 189 | + | 246 | + | 303 | ++ |
| 19 | | 76 | + | 133 | | 190 | + | 247 | + | 304 | ++ |
| 20 | + | 77 | | 134 | | 191 | | 248 | + | 305 | + |
| 21 | | 78 | | 135 | | 192 | | 249 | | 306 | ++ |
| 22 | ++ | 79 | | 136 | | 193 | | 250 | | 307 | +++ |
| 23 | + | 80 | | 137 | | 194 | | 251 | + | 308 | + |
| 24 | ++ | 81 | + | 138 | | 195 | | 252 | ++ | 309 | +++ |
| 25 | + | 82 | | 139 | | 196 | | 253 | ++ | 310 | + |
| 26 | ++ | 83 | + | 140 | ++ | 197 | | 254 | ++ | 311 | + |
| 27 | ++ | 84 | + | 141 | | 198 | + | 255 | + | 312 | |
| 28 | + | 85 | + | 142 | ++ | 199 | ++ | 256 | + | 313 | ++ |
| 29 | + | 86 | ++ | 143 | + | 200 | + | 257 | + | 314 | ++ |
| 30 | | 87 | + | 144 | | 201 | + | 258 | ++ | 315 | + |
| 31 | + | 88 | + | 145 | | 202 | + | 259 | ++ | 316 | + |
| 32 | + | 89 | + | 146 | | 203 | + | 260 | ++ | 317 | + |
| 33 | ++ | 90 | + | 147 | | 204 | + | 261 | + | 318 | + |
| 34 | ++ | 91 | | 148 | | 205 | + | 262 | | 319 | + |
| 35 | + | 92 | + | 149 | | 206 | + | 263 | ++ | 320 | + |
| 36 | ++ | 93 | | 150 | + | 207 | + | 264 | ++ | 321 | + |
| 37 | + | 94 | | 151 | ++ | 208 | + | 265 | + | 322 | + |
| 38 | + | 95 | | 152 | + | 209 | + | 266 | ++ | 323 | + |
| 39 | + | 96 | | 153 | + | 210 | ++ | 267 | ++ | 324 | ++ |
| 40 | ++ | 97 | + | 154 | + | 211 | ++ | 268 | + | 325 | |
| 41 | +++ | 98 | | 155 | + | 212 | + | 269 | + | 326 | + |
| 42 | ++ | 99 | + | 156 | + | 213 | +++ | 270 | ++ | 327 | |
| 43 | + | 100 | + | 157 | | 214 | ++ | 271 | | 328 | ++ |
| 44 | + | 101 | + | 158 | + | 215 | +++ | 272 | ++ | 329 | ++ |
| 45 | | 102 | + | 159 | | 216 | ++ | 273 | + | 330 | ++ |
| 46 | | 103 | + | 160 | | 217 | ++ | 274 | + | 331 | + |
| 47 | + | 104 | + | 161 | + | 218 | ++ | 275 | ++ | 332 | ++ |
| 48 | | 105 | + | 162 | | 219 | ++ | 276 | ++ | 333 | |
| 49 | + | 106 | ++ | 163 | | 220 | + | 277 | ++ | 334 | +++ |
| 50 | | 107 | ++ | | | 221 | + | 278 | + | 335 | +++ |
| 51 | + | 108 | + | 165 | | 222 | + | 279 | + | 336 | +++ |
| 52 | | 109 | | 166 | | 223 | + | 280 | + | 337 | +++ |
| 53 | + | 110 | ++ | 167 | | 224 | + | 281 | + | 338 | + |
| 54 | + | 111 | + | 168 | | 225 | ++ | 282 | + | | |
| 55 | + | 112 | + | 169 | | 226 | + | 283 | + | | |
| 56 | | 113 | ++ | | | 227 | + | 284 | + | | |
| 57 | + | 114 | ++ | 171 | | 228 | ++ | 285 | + | | |
| 339 | + | 361 | ++ | 383 | ++ | 405 | + | 427 | ++ | 449 | ++ |
| 340 | + | 362 | + | 384 | ++ | 406 | + | 428 | ++ | 450 | ++ |
| 341 | + | 363 | ++ | 385 | + | 407 | + | 429 | ++ | 451 | + |
| 342 | ++ | 364 | + | 386 | +++ | 408 | + | 430 | ++ | 452 | ++ |
| 343 | ++ | 365 | ++ | 387 | ++ | 409 | ++ | 431 | ++ | 453 | +++ |
| 344 | ++ | 366 | ++ | 388 | + | 410 | + | 432 | − | 454 | ++ |
| 345 | +++ | 367 | + | 389 | + | 411 | ++ | 433 | + | 455 | + |
| 346 | +++ | 368 | ++ | 390 | + | 412 | + | 434 | ++ | 456 | ++ |
| 347 | ++ | 369 | ++ | 391 | + | 413 | ++++ | 435 | ++ | 457 | ++ |
| 348 | ++ | 370 | + | 392 | + | 414 | + | 436 | + | 458 | ++ |
| 349 | + | 371 | + | 393 | +++ | 415 | + | 437 | + | 459 | + |
| 350 | ++ | 372 | + | 394 | ++ | 416 | + | 438 | ++ | 460 | + |
| 351 | + | 373 | +++ | 395 | | 417 | + | 439 | ++ | 461 | +++ |
| 352 | ++ | 374 | ++ | 396 | ++ | 418 | ++ | 440 | ++ | 462 | + |
| 353 | + | 375 | +++ | 397 | ++ | 419 | + | 441 | ++ | 463 | ++ |
| 354 | ++ | 376 | + | 398 | + | 420 | + | 442 | + | 464 | |
| 355 | ++ | 377 | +++ | 399 | +++ | 421 | ++ | 443 | + | 465 | |
| 356 | ++ | 378 | + | 400 | ++ | 422 | + | 444 | ++ | 466 | |
| 357 | + | 379 | +++ | 401 | + | 423 | + | 445 | +++ | 467 | |
| 358 | + | 380 | ++ | 402 | + | 424 | + | 446 | ++ | 468 | |
| 359 | ++ | 381 | + | 403 | ++ | 425 | ++ | 447 | + | 469 | |
| 360 | ++ | 382 | + | 404 | + | 426 | + | 448 | ++ | 470 | |
| | | | | | | | | | | 471 | |
| | | | | | | | | | | 472 | |
| | | | | | | | | | | 473 | |
| | | | | | | | | | | 474 | + |
| | | | | | | | | | | 475 | + |
| | | | | | | | | | | 476 | + |

| Compound # | hPIKfyve BRET IC₅₀ (μM) |
|---|---|
| 14 | +++ |
| 159 | +++ |
| 179 | +++ |
| 296 | ++ |
| 136 | + |
| 98 | ++ |
| 325 | ++++ |
| 333 | ++++ |
| 118 | +++ |
| 119 | ++++ |

### Example 3. Viability Assay to Assess TDP-43 Toxicity in FAB1 TDP-43 and PIKfyve TDP-43 Yeast Cells

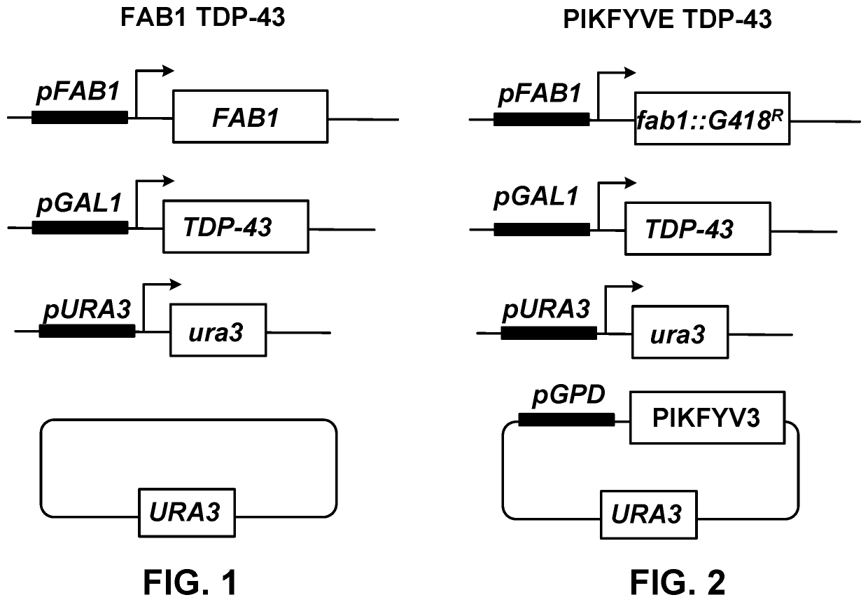

Generation of TDP-43 yeast model expressing human PIKfyve. Human PIKFYVE (“entry clone”) was cloned into pAG416GPDccdB (“destination vector”) according to standard Gateway cloning protocols (Invitrogen, Life Technologies). The resulting pAG416GPD-PIKFYVE plasmids were amplified in E. coli and plasmid identity confirmed by restriction digest and Sanger sequencing. Lithium acetate/polyethylene glycol-based transformation was used to introduce the above PIKFYVE plasmid into a BY4741 yeast strain auxotrophic for the ura3 gene and deleted for two transcription factors that regulate the xenobiotic efflux pumps, a major efflux pump, and FAB1, the yeast ortholog of PIKFYVE (MATa, snq2::KILeu2; pdr3::Klura3;pdR¹::NATMX; fab1::G418^{R}, his3;leu2;ura3;met15;LYS2+) (FIG. 2). Transformed yeast were plated on solid agar plates with complete synthetic media lacking uracil (CSM-ura) and containing 2% glucose. Individual colonies harboring the control or PIKFYVE TDP-43 plasmids were recovered. A plasmid containing wild-type TDP-43 under the transcriptional control of the GAL1 promoter and containing the hygromycin-resistance gene as a selectable marker was transformed into the fab1::G418^{R} pAG416GPD-PIKFYVE yeast strain (FIG. 1). Transformed yeast were plated on CSM-ura containing 2% glucose and 200 g/mL G418 after overnight recovery in media lacking antibiotic. Multiple independent isolates were further evaluated for cytotoxicity and TDP-43 expression levels.

Viability Assay. A control yeast strain with the wild-type yeast FAB1 gene and TDP-43 (“FAB1 TDP-43”, carries empty pAG416 plasmid), and the “PIKFYVE TDP-43” yeast strain, were assessed for toxicity using a propidium iodide viability assay. Both yeast strains were transferred from solid CSM-ura/2% glucose agar plates into 3 mL of liquid CSM-ura/2% glucose media for 6-8 hours at 30° C. with aeration. Yeast cultures were then diluted to an optical density at 600 nm wavelength (OD₆₀₀) of 0.005 in 3 mL of CSM-ura/2% raffinose and grown overnight at 30° C. with aeration to an OD₆₀₀ of 0.3-0.8. Log-phase overnight cultures were diluted to OD₆₀₀ of 0.005 in CSM-ura containing either 2% raffinose or galactose and 150 L dispensed into each well of a flat bottom 96-well plates. Compounds formulated in 100% dimethyl sulfoxide (DMSO) were serially diluted in DMSO and 1.5 L diluted compound transferred to the 96-well plates using a multichannel pipet. Wells containing DMSO alone were also evaluated as controls for compound effects. Tested concentrations ranged from 15 M to 0.11 M. Cultures were immediately mixed to ensure compound distribution and covered plates incubated at 30° C. for 24 hours in a stationary, humified incubator.

Upon the completion of incubation, cultures were assayed for viability using propidium iodide (PI) to stain for dead/dying cells. A working solution of PI was made where, for each plate, 1 L of 10 mM PI was added to 10 mL of CSM-ura (raffinose or galactose). The final PI solution (50 L/well) was dispensed into each well of a new round bottom 96-well plate. The overnight 96-well assay plate was then mixed with a multichannel pipet and 50 L transferred to the PI-containing plate. This plate was then incubated for 30 minutes at 30° C. in the dark. A benchtop flow cytometer (Miltenyi MACSquant) was then used to assess red fluorescence (B2 channel), forward scatter, and side scatter (with following settings: gentle mix, high flow rate, fast measurement, 10,000 events). Intensity histograms were then gated for “PI-positive” or “PI-negative” using the raffinose and galactose cultures treated with DMSO as controls. The DMSO controls for raffinose or galactose-containing cultures were used to determine the window of increased cell death and this difference set to 100. All compounds were similarly gated and then compared to this maximal window to establish the percent reduction in PI-positive cells. IC50 values were then calculated for compounds that demonstrated a concentration-dependent enhancement of viability by fitting a logistic regression curve.

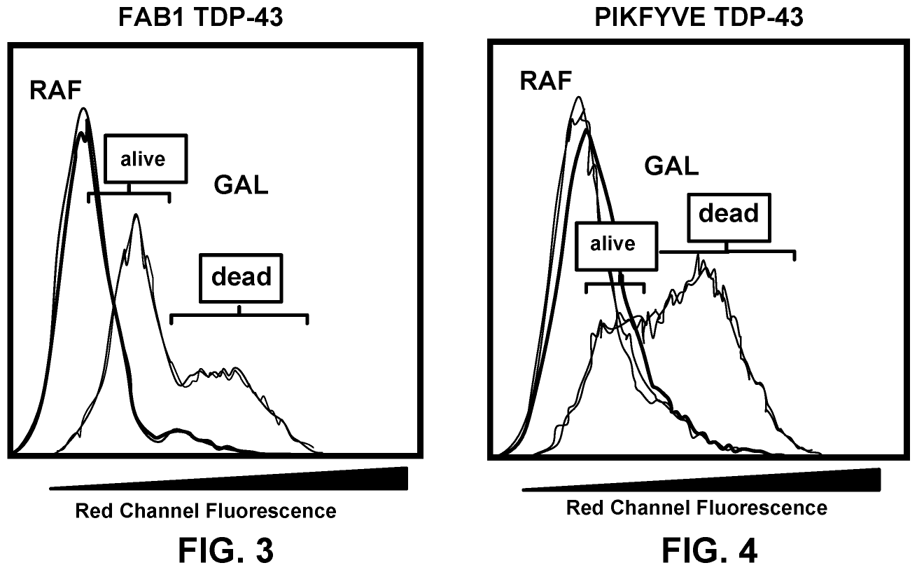

Upon induction of TDP-43 in both strains, there was a marked increase in inviable cells (rightmost population) with both FAB1 TDP-43 and PIKFYVE TDP-43, with a more pronounced effect in PIKFYVE TDP-43 (FIGS. 3 and 4).

PIKfyve Inhibition Suppresses Toxicity in PIKfyve TDP-43 Model. The biochemical PIKFyve inhibition assays were run by Carna Biosciences according to proprietary methodology based on the Promega ADP-Glo™ Kinase assay. A full-length human PIKFYVE [1-2098(end) amino acids and S696N, L932S, Q995L, T998S, S1033 A and Q1183K of accession number NP_055855.2] was expressed as N-terminal GST-fusion protein (265 kDa) using baculovirus expression system. GST-PIKFYVE was purified by using glutathione sepharose chromatography and used in an ADP-Glo™ Kinase assay (Promega). Reactions were set up by adding the test compound solution, substrate solution, ATP solution and kinase solution, each at 4× final concentrations. Reactions were prepared with assay buffer (50 mM MOPS, 1 mM DTT, pH7.2), mixed, and incubated in black 384 well polystyrene plates for 1 hour at room temperature. ADP-Glo™ reagent was then added for 40 minutes, followed by kinase detection reagent for an additional 40 minutes. The kinase activity was evaluated by detecting relative light units on a luminescence plate reader. Samples were run in duplicate from 10 uM to 3 nM. Data was analyzed by setting the control wells (+PIKfyve, no compound) to 0% inhibition and the readout value of background (no PIKfyve) set to 100% inhibition, then the % inhibition of each test solution calculated. IC50 values were calculated from concentration vs % inhibition curves by fitting to a four-parameter logistic curve.

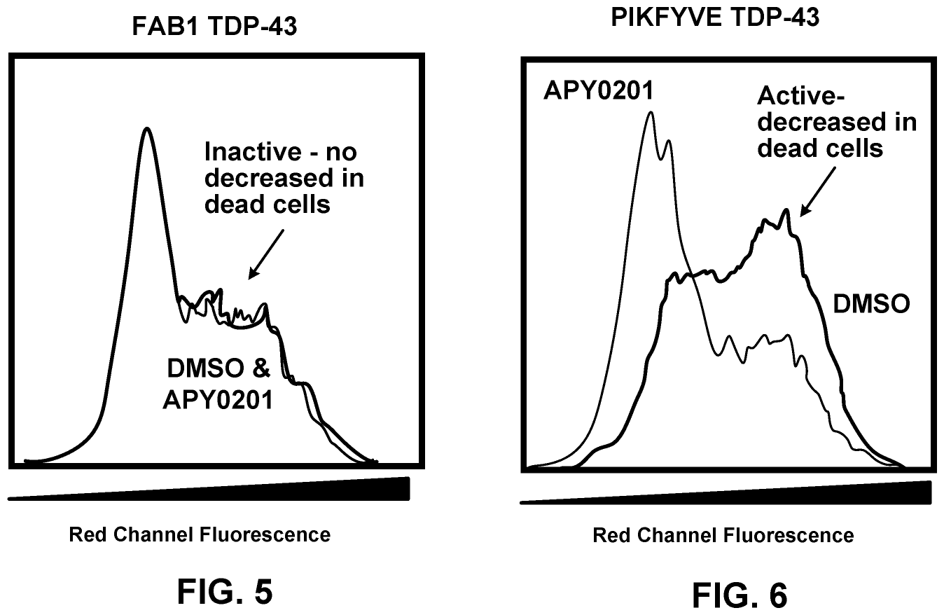

Activity of APYO201, a known PIKFYVE inhibitor, in FAB1 TDP-43 (FIG. 5) and PIKFYVE TDP-43 (FIG. 6). There was no increase in viable cells in FAB1 TDP-43 across a range of compound concentrations as evidenced by a lack in reduction of the right most population of propidium iodide-positive cells (only 0.23 μM is shown). In the PIKFYVE TDP-43 model, 0.23 μM reduced the population of propidium iodide-positive dead cells, indicating PIKFYVE inhibition ameliorated TDP-43 toxicity. Concentrations ranging from 0.5 mM to less than 100 nM afforded increased viability.

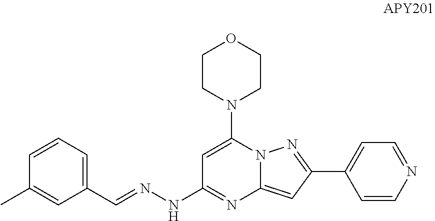 [SMILES: Cc1cccc(/C=N/Nc2cc(N3CCOCC3)n3nc(-c4ccncc4)cc3n2)c1]

A panel of compounds was tested in a biochemical PIKFYVE assay (ADP-Glo™ with full-length PIKfyve) and IC50's determined (nM) (see the Table below). The same compounds were also tested in both FAB1 and PIKFYVE TDP-43 yeast models. Their activity is reported here as “active” or “inactive.” Compounds with low nanomolar potency in the biochemical assay were active in the PIKFYVE TDP-43 yeast model. Compounds that were less potent or inactive in the biochemical assay were inactive in the PIKFYVE TDP-43 model. Compounds that were inactive in the biochemical or PIKFYVE TDP-43 assays were plotted with the highest concentrations tested in that assay.

| | PIKfyve IC₅₀ | FAB1 TDP-43 | PIKfyve TDP-43 |
|---|---|---|---|
| Structure | (nM) | (active/inactive) | (active/inactive) |
| 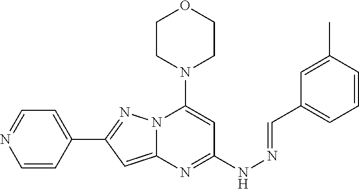 [SMILES: Cc1cccc(/C=N/Nc2cc(N3CCOCC3)n3nc(-c4ccncc4)cc3n2)c1] | 7.5 | Inactive | Active |
| 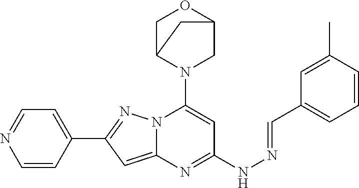 [SMILES: Cc1cccc(/C=N/Nc2cc(N3CC4CC3CO4)n3nc(-c4ccncc4)cc3n2)c1] | 12 | Inactive | Active |
| 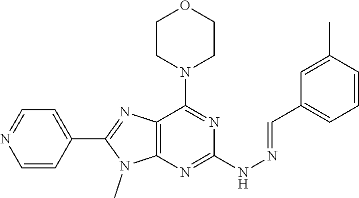 [SMILES: Cc1cccc(/C=N/Nc2nc(N3CCOCC3)c3nc(-c4ccncc4)n(C)c3n2)c1] | 4.9 | Inactive | Active |
| 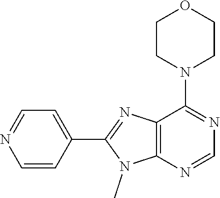 [SMILES: Cn1c(-c2ccncc2)nc2c(N3CCOCC3)ncnc21] | 640 | Inactive | Inactive |
| 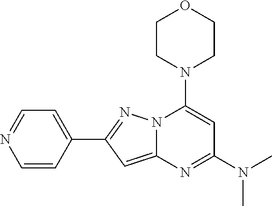 [SMILES: CN(C)c1cc(N2CCOCC2)n2nc(-c3ccncc3)cc2n1] | 2007 | Inactive | Inactive |
| 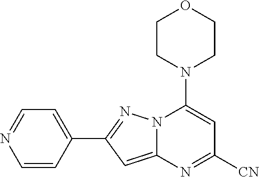 [SMILES: N#Cc1cc(N2CCOCC2)n2nc(-c3ccncc3)cc2n1] | >10000 | Inactive | Inactive |

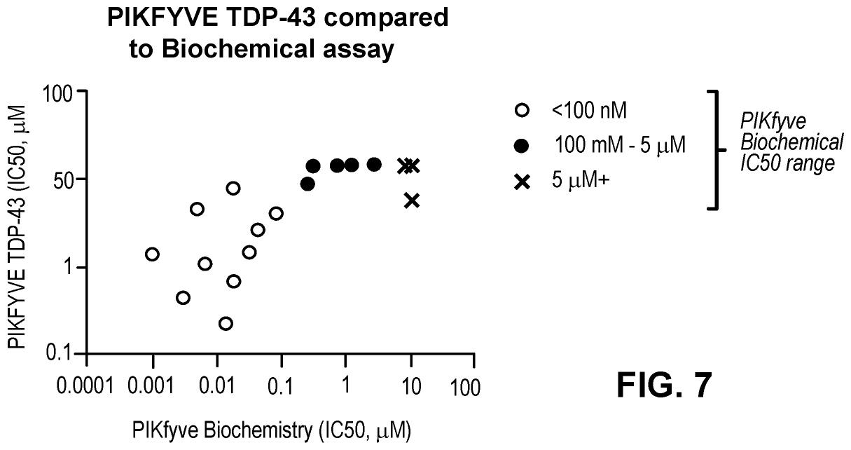

Biochemical and Efficacy Assays. A larger set of PIKfyve inhibitors were evaluated in both a PIKfyve kinase domain binding assay (nanobret) and in the PIKFYVE TDP-43 yeast strain. IC50 values (μM) were plotted. Data points are formatted based on binned potency from the nanobret assay as indicated in the legend (FIG. 7). Below is a table of compounds and their biochemical and PIKFYVE TDP-43 IC50 values plotted in FIG. 7.

| | PIKFYVE Biochemistry | PIKFYVE TDP-43 |
|---|---|---|
| Structure | (IC50, μM) | (IC50, μM) |
| 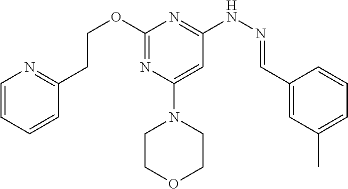 [SMILES: Cc1cccc(/C=N/Nc2cc(N3CCOCC3)nc(OCCc3ccccn3)n2)c1] | 0.003 | 0.450 |
| 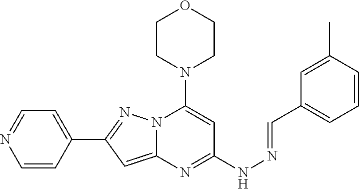 [SMILES: Cc1cccc(/C=N/Nc2cc(N3CCOCC3)n3nc(-c4ccncc4)cc3n2)c1] | 0.001 | 1.390 |
| 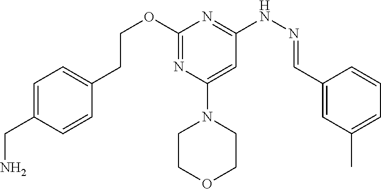 [SMILES: Cc1cccc(/C=N/Nc2cc(N3CCOCC3)nc(OCCc3ccc(CN)cc3)n2)c1] | 0.007 | 1.120 |
| 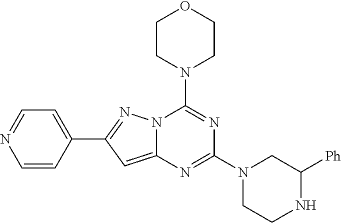 [SMILES: c1ccc(C2CN(c3nc(N4CCOCC4)n4nc(-c5ccncc5)cc4n3)CCN2)cc1] | 2.660 | >15 |
| 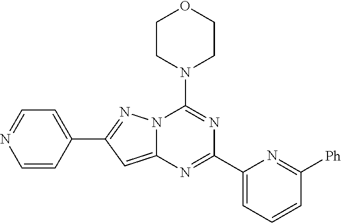 [SMILES: c1ccc(-c2cccc(-c3nc(N4CCOCC4)n4nc(-c5ccncc5)cc4n3)n2)cc1] | 0.014 | 0.230 |
| 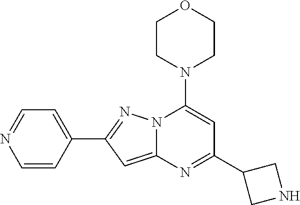 [SMILES: c1cc(-c2cc3nc(C4CNC4)cc(N4CCOCC4)n3n2)ccn1] | 8.020 | >15 |
| 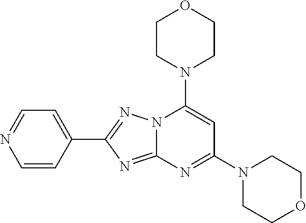 [SMILES: c1cc(-c2nc3nc(N4CCOCC4)cc(N4CCOCC4)n3n2)ccn1] | 9.200 | >15 |
| 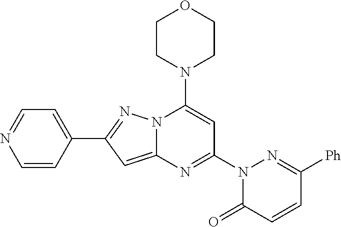 [SMILES: O=c1ccc(-c2ccccc2)nn1-c1cc(N2CCOCC2)n2nc(-c3ccncc3)cc2n1] | 0.295 | >15 |
| 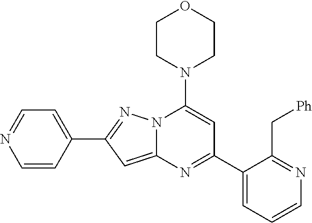 [SMILES: c1ccc(Cc2ncccc2-c2cc(N3CCOCC3)n3nc(-c4ccncc4)cc3n2)cc1] | 1.090 | >15 |
| 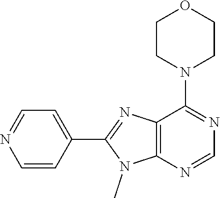 [SMILES: Cn1c(-c2ccncc2)nc2c(N3CCOCC3)ncnc21] | 0.640 | >15 |
| 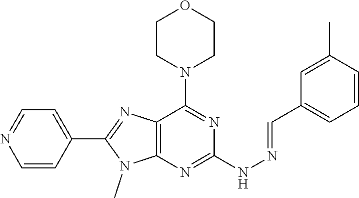 [SMILES: Cc1cccc(/C=N/Nc2nc(N3CCOCC3)c3nc(-c4ccncc4)n(C)c3n2)c1] | 0.005 | 4.720 |
| 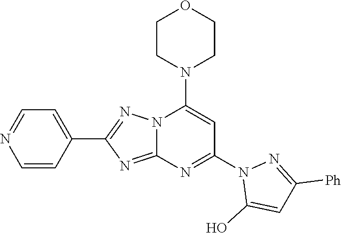 [SMILES: Oc1cc(-c2ccccc2)nn1-c1cc(N2CCOCC2)n2nc(-c3ccncc3)nc2n1] | 0.018 | 0.693 |
| 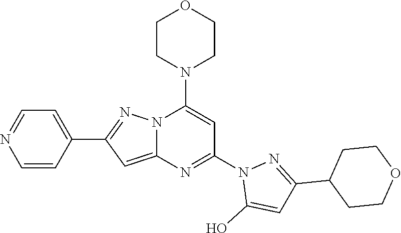 [SMILES: Oc1cc(C2CCOCC2)nn1-c1cc(N2CCOCC2)n2nc(-c3ccncc3)cc2n1] | 0.253 | 9.105 |
| 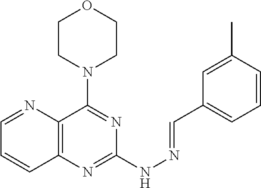 [SMILES: Cc1cccc(/C=N/Nc2nc(N3CCOCC3)c3ncccc3n2)c1] | 0.018 | 8.214 |
| 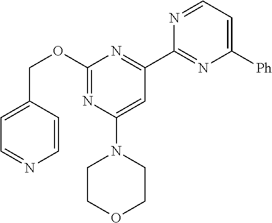 [SMILES: c1ccc(-c2ccnc(-c3cc(N4CCOCC4)nc(OCc4ccncc4)n3)n2)cc1] | 0.032 | 1.447 |
| 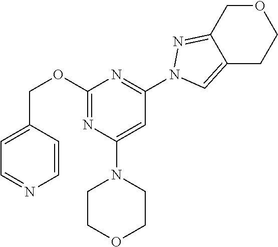 [SMILES: c1cc(COc2nc(N3CCOCC3)cc(-n3cc4c(n3)COCC4)n2)ccn1] | 1.343 | >15 |
| 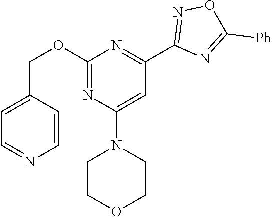 [SMILES: c1ccc(-c2nc(-c3cc(N4CCOCC4)nc(OCc4ccncc4)n3)no2)cc1] | >10 | >15 |
| 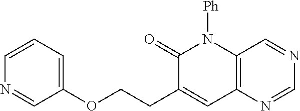 [SMILES: O=c1c(CCOc2cccnc2)cc2ncncc2n1-c1ccccc1] | >10 | >15 |
| 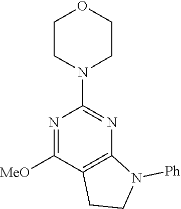 [SMILES: COc1nc(N2CCOCC2)nc2c1CCN2c1ccccc1] | 0.085 | 4.273 |
| 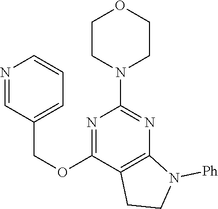 [SMILES: c1ccc(N2CCc3c(OCc4cccnc4)nc(N4CCOCC4)nc32)cc1] | 0.042 | 2.685 |
| 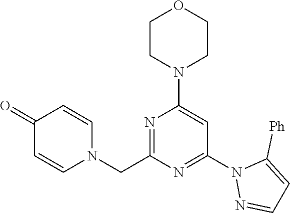 [SMILES: O=c1ccn(Cc2nc(N3CCOCC3)cc(-n3nccc3-c3ccccc3)n2)cc1] | >10 | >15 |
| 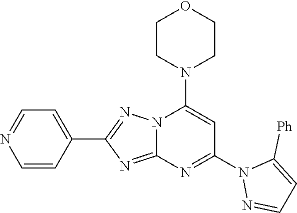 [SMILES: c1ccc(-c2ccnn2-c2cc(N3CCOCC3)n3nc(-c4ccncc4)nc3n2)cc1] | 0.767 | >15 |
| 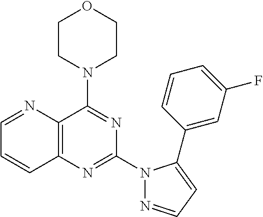 [SMILES: Fc1cccc(-c2ccnn2-c2nc(N3CCOCC3)c3ncccc3n2)c1] | >10 | 5.754 |

### OTHER EMBODIMENTS

Various modifications and variations of the described invention will be apparent to those skilled in the art without departing from the scope and spirit of the invention. Although the invention has been described in connection with specific embodiments, it should be understood that the invention as claimed should not be unduly limited to such specific embodiments. Indeed, various modifications of the described modes for carrying out the invention that are obvious to those skilled in the art are intended to be within the scope of the invention.

Other embodiments are in the claims.

1. A compound of formula (1): 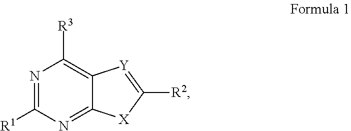
or a pharmaceutically acceptable salt thereof,
wherein
X is NR^{A};
Y is CR^{A} or N;
R¹ is optionally substituted C₁-C₁₀ heteroaryl comprising a 5-membered ring having a nitrogen atom at position 2 relative to the bond to the core; 4,5-dihydropyrazol-1-yl substituted with phenyl; optionally substituted pyrimidin-2-yl, optionally substituted pyridazin-6-yl, optionally substituted pyrimidin-4-yl; pyridin-3-yl optionally substituted with methoxy; optionally substituted indazol-1-yl; optionally substituted indazol-2-yl; optionally substituted indazol-7-yl; optionally substituted isoindolin-6-yl; optionally substituted pyridazin-5-yl; optionally substituted pyrrolidine-1-yl; optionally substituted pyrimidin-6-yl; optionally substituted piperazinyl; phenyl substituted with methoxy, optionally substituted C₁-C₆ alkyl, hydroxyl, optionally substituted C₂-C₉ heteroaryl, optionally substituted C6-C₁₀ aryl, optionally substituted C₂-C₉ heterocyclyl, or C₃-C₈ cycloalkoxy; optionally substituted C₃ carbocyclyl; optionally substituted morpholin-1-yl; optionally substituted benzodioxolyl; optionally substituted benzopyrrolidonyl; optionally substituted tetrahydroquinoline; optionally substituted monoalkylamino; optionally substituted dialkylamino; amino monosubstituted with optionally substituted C₂-C₉ heteroaryl; halo; optionally substituted C₂-C₉ heterocycle C₁ alkyl; optionally substituted C₂-C₉ heteroaryl C₁ alkyl; optionally substituted benzodioxanyl; —NHNHR^{1A}; —N(R^{1A})N═C(R^{1B})₂; —C(R^{1A})═N—N(R^{1B})₂; —C(R^{1A})═NOR^{1A}; or 1-Q-N(R^{1C})₂;
Q¹ is a bond, CH₂, or CO;
each R^{1A} is independently H, optionally substituted C₁-C₆ alkyl, optionally substituted C₆-C₁₀ aryl, or optionally substituted C₆-C₁₀ aryl C₁-C₆ alkyl;
one R^{1B} is independently H, optionally substituted C₁-C₆ alkyl, optionally substituted C₆-C₁₀ aryl, or optionally substituted C₂-C₉ heteroaryl; and the remaining R^{1B} is optionally substituted C₁-C₆ alkyl, optionally substituted C₆-C₁₀ aryl, or optionally substituted C₂-C₉ heteroaryl;
each R^{1C} is independently H, optionally substituted C₁-C₆ alkyl, optionally substituted C₆-C₁₀ aryl, optionally substituted C₃-C₈ cycloalkyl, or optionally substituted C₂-C₉ heteroaryl;
or both R^{1C}, together with the nitrogen atom to which they are attached, combine to form C₂-C₉ heterocyclyl or C₂-C₉ heteroaryl;
R² is H, halogen, optionally substituted C₆-C₁₀ aryl; optionally substituted C₁0.9 heterocyclyl; —O-pyridin-3-yl; optionally substituted C₃-C₈ cycloalkyl; optionally substituted C₃-C₈ cycloalkenyl, C₁-C₂ alkyl optionally substituted with hydroxy, methoxy, —CH₂OH, pyridin-4-yl, 4-pyridon-1-yl, —O-pyridin-4-yl, oxo, or dialkyl amino; C₁ alkyl optionally substituted with deuterium, oxo, hydroxy, halo, or amino substituted with C₃ cycloalkyl; C₃ alkyl substituted with hydroxy, oxo, or dialkyl amino; C₄ alkyl; optionally substituted C₂-C₉ heteroaryl; -Q-N(Ric)₂; —S(O)ᵣ—R^{1A}; or —P(O)(R^{1A})₂; and each R^{A} is independently H, C₁-C₂ alkyl optionally substituted with hydroxyl or —S(O)ᵣ-(optionally substituted C₁-C₆ alkyl), C₃ alkyl, C₄-C₅ alkyl substituted with hydroxyl, optionally substituted C₂-C₉ heteroaryl C₁-C₆ alkyl; optionally substituted C₃-C₈ cycloalkyl, optionally substituted C₃-C₈ cycloalkyl C₁-C₆ alkyl, optionally substituted C₆-C₁₀ aryl, or optionally substituted C₂-C₉ heteroaryl; or R² and R^{A}, together with the atoms to which they are attached, combine to form an optionally substituted C₃-C₄ heterocyclic ring, and the remaining R^{A}, if present, is H, C₁-C₂ alkyl optionally substituted with hydroxyl or —S(O)ᵣ (optionally substituted C₁-C₆ alkyl), C₃ alkyl, C₄-C₅ alkyl substituted with hydroxyl, optionally substituted C₂-C₉ heteroaryl C₁-C₆ alkyl; optionally substituted C₃-C₈ cycloalkyl, optionally substituted C₃-C₈ cycloalkyl C₁-C₆ alkyl, optionally substituted C₆-C₁₀ aryl, or optionally substituted C₂-C₉ heteroaryl;
r is 0, 1, or 2; and
R³ is

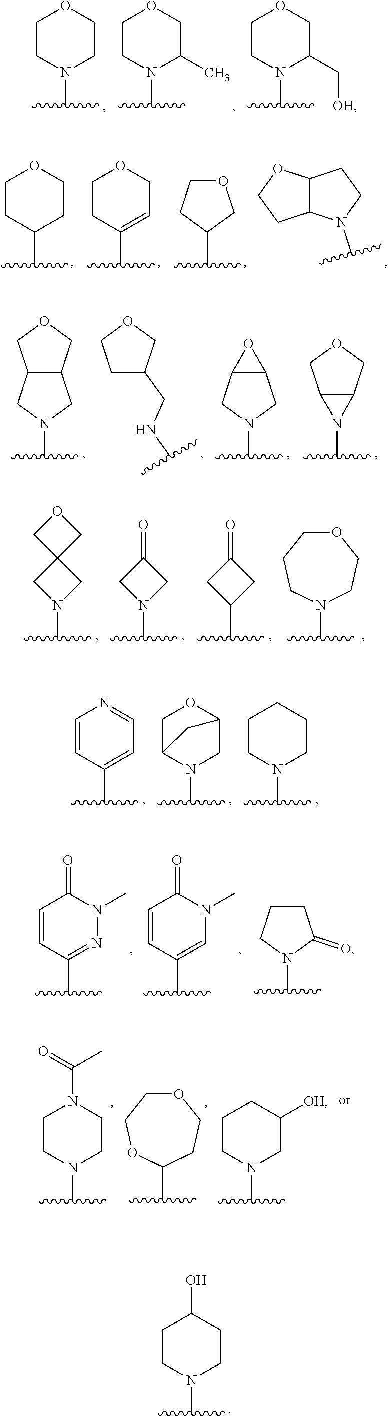

2. (canceled)

3. The compound of claim 1, wherein Y is N.

4-6. (canceled)

7. The compound of claim 1, wherein R^{A} is H, C₁-C₂ alkyl optionally substituted with hydroxyl or —S(O)CH₃, C₃ alkyl, C₄-C₅ alkyl substituted with hydroxyl.

8-9. (canceled)

10. The compound of claim 1, wherein R¹ is optionally substituted pyrazol-1-yl, optionally substituted pyrazol-3-yl, optionally substituted 1,2,3-triazol-1-yl, optionally substituted 1,2,3-traizol-2-yl, optionally substituted benzotriazole-1-yl, optionally substituted 1,2,4 triazol-3-yl, optionally substituted 1,2,4-oxadizol-3-yl, or optionally substituted 1,2,4-oxadizol-2-yl.

11-13. (canceled)

14. The compound of claim 1, wherein R¹ is

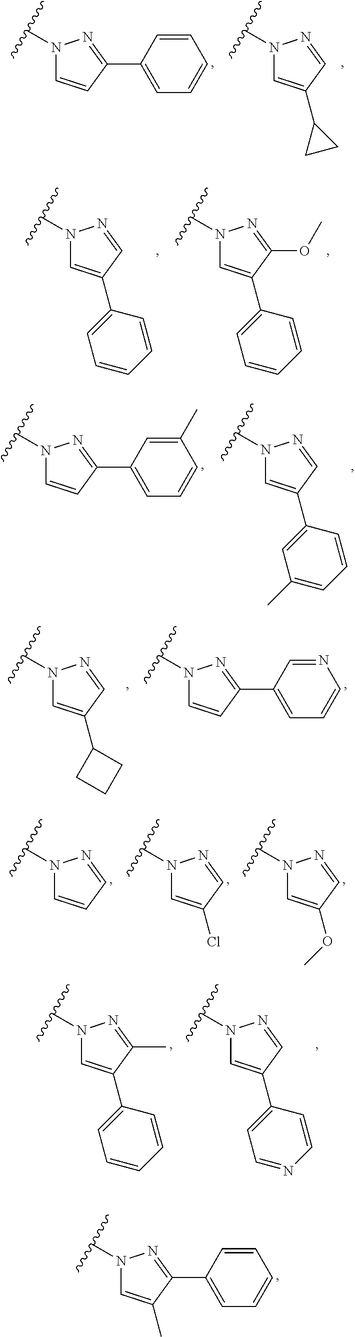

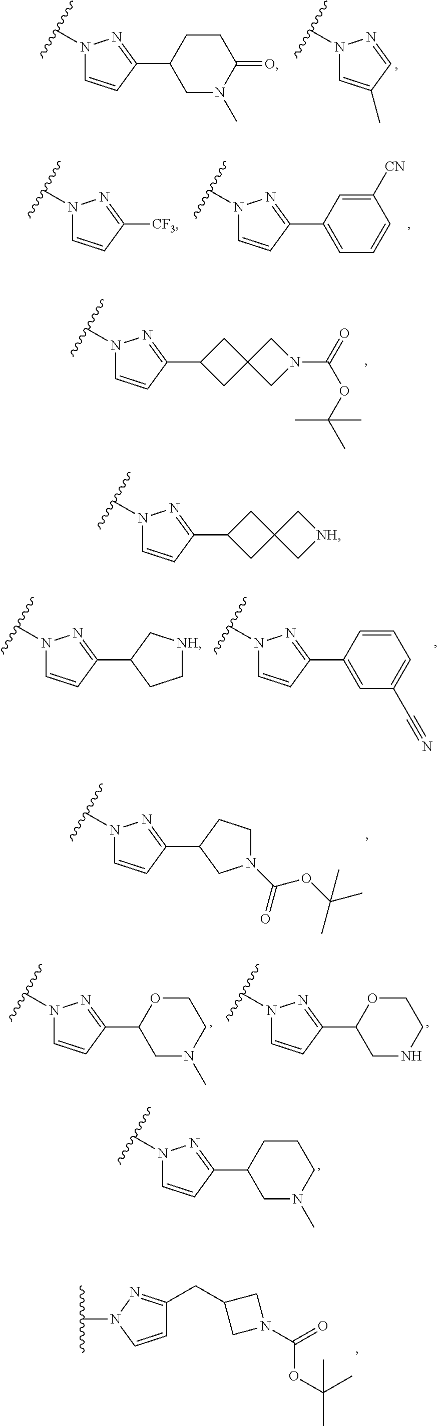

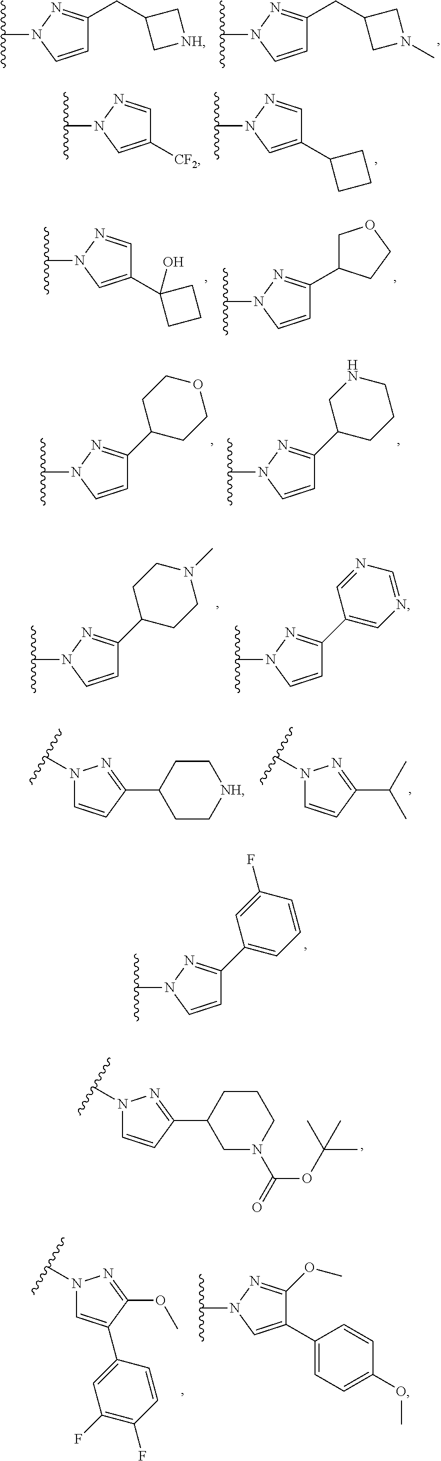

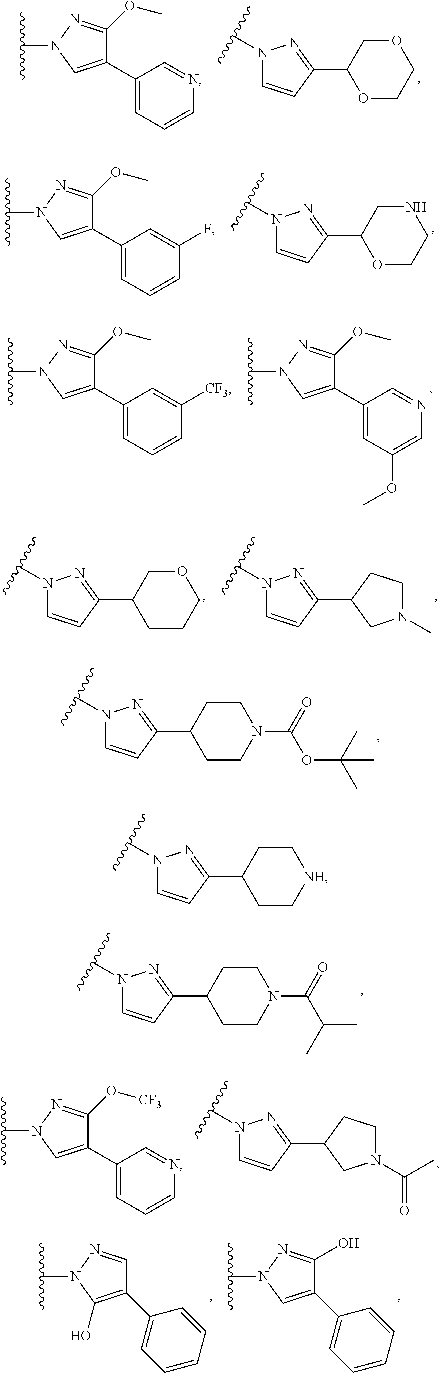

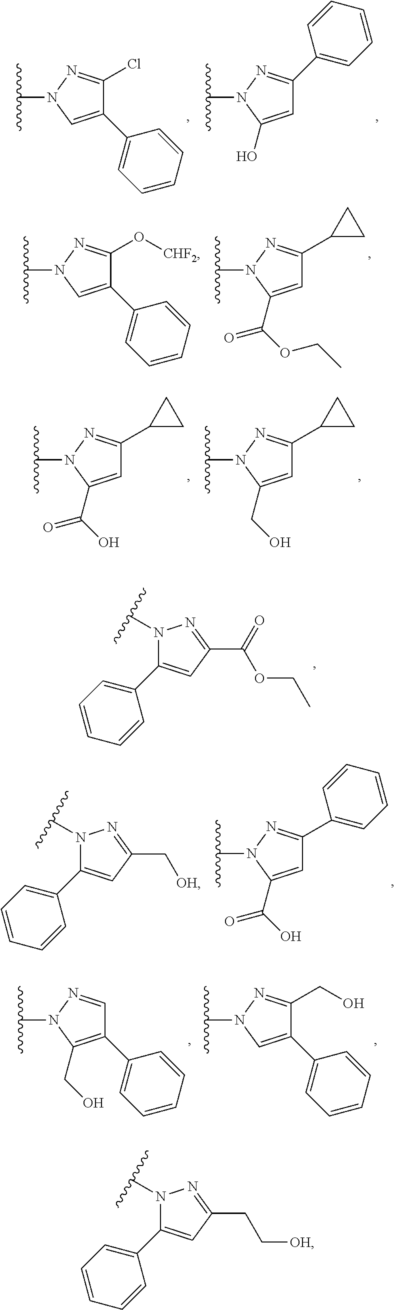

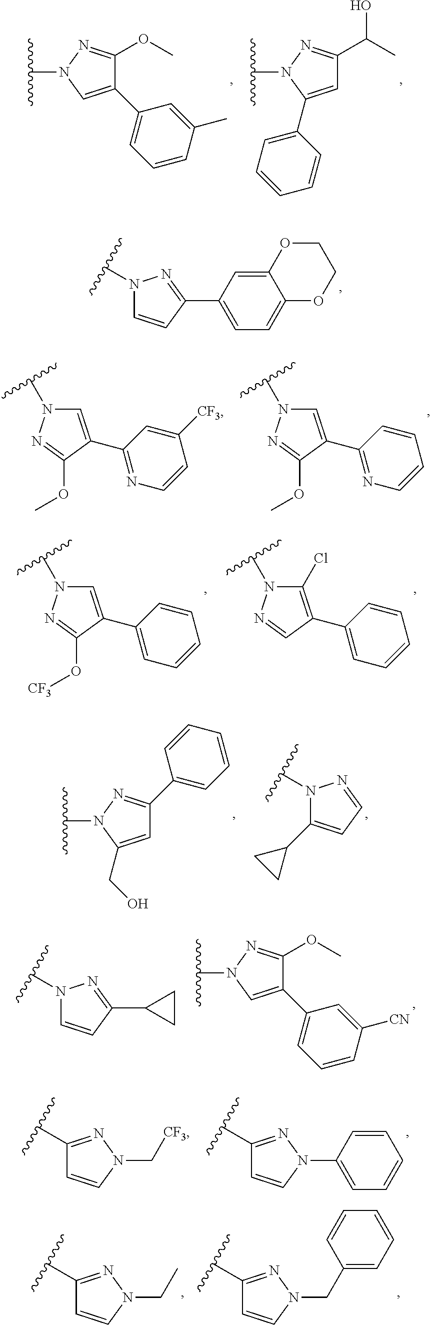

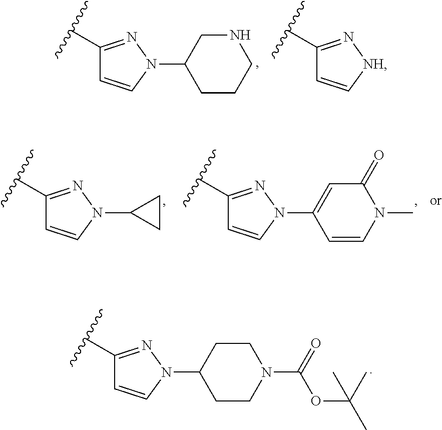

15-20. (canceled)

21. The compound of claim 1, wherein R¹ is

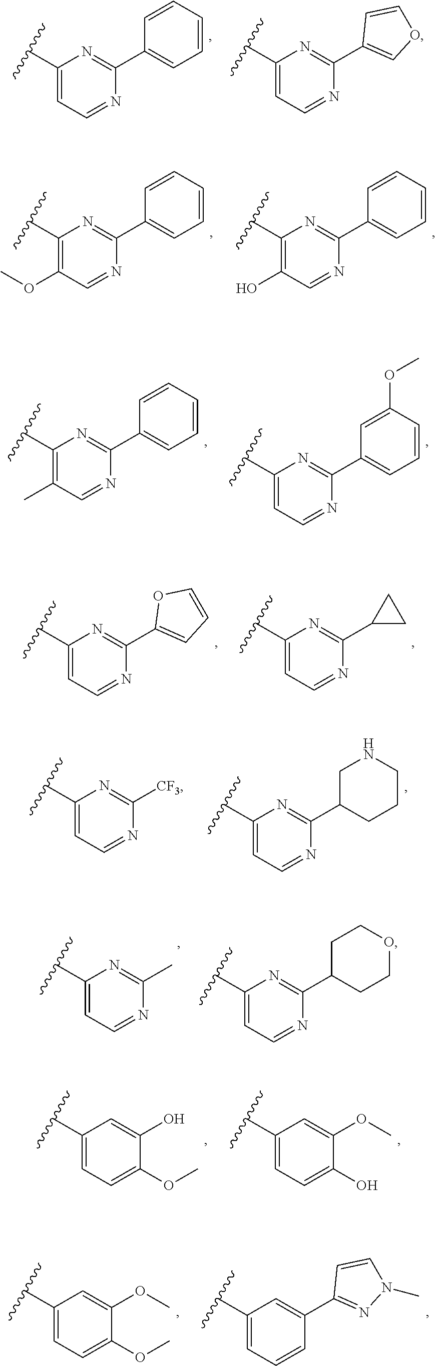

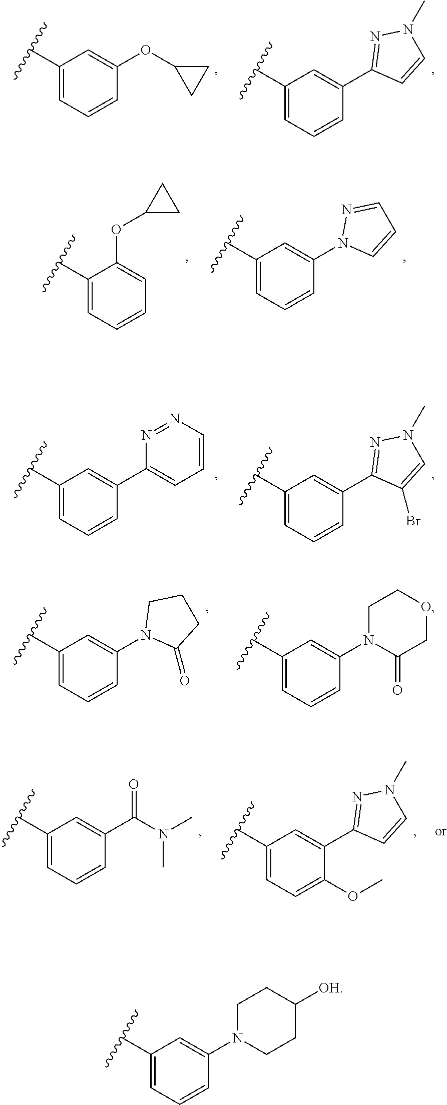

22-24. (canceled)

25. The compound of claim 1, wherein R² is optionally substituted C₂-C₉ heteroaryl.

26. The compound of claim 1, wherein R² is optionally substituted pyridyl.

27. (canceled)

28. The compound of claim 1, wherein R² is optionally substituted tetrahydropyranyl, optionally substituted dihydropyranyl, optionally substituted piperidinyl, or optionally substituted azetidinyl.

29. (canceled)

30. The compound of claim 1, wherein R^{1A} is substituted with oxo.

31. The compound of claim 1, wherein
the compound has the structure: 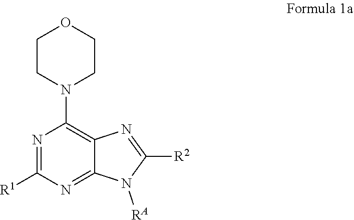
or a pharmaceutically acceptable salt thereof;
or the compound has the structure: 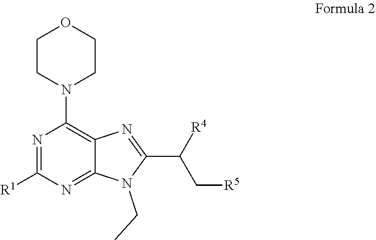
or a pharmaceutically acceptable salt thereof,
wherein R¹ is optionally substituted pyrazol-1-yl, phenyl substituted with optionally substituted C₂-C₉ heteroaryl, or optionally substituted pyridimin-4-yl; and
R⁴ and R⁵ are each, independently, hydroxyl or methoxy;
or the compound has the structure: 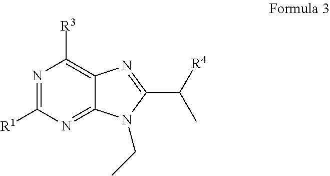
or a pharmaceutically acceptable salt thereof,
wherein R¹ is optionally substituted pyrazol-1-yl, phenyl substituted with optionally substituted heteroaryl, optionally substituted indazol-1-yl, or optionally substituted indazol-2-yl;
R⁴ is hydroxyl, 4-pyridinon-1-yl, —O-pyridin-3-yl, or CH₂OH; and
R³ is pyridin-4-yl or morpholin-1-yl;
or the compound has the structure: 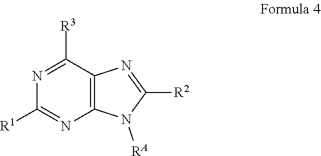
or a pharmaceutically acceptable salt thereof,
wherein R¹ is phenyl optionally substituted with methoxy or optionally substituted heteroaryl or optionally substituted pyrazol-1-yl,
R³ is morpholin-1-yl or piperidin-1-yl; and
R² is 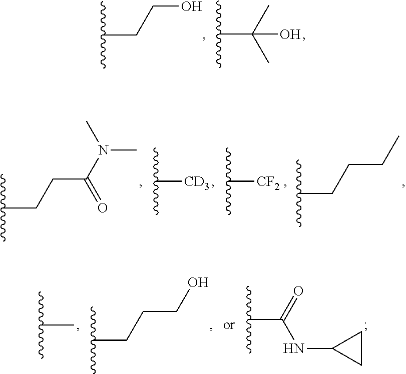
and
R^{A} is ethyl, 2-hydroxy-ethyl, or 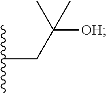 [SMILES: *CC(C)(C)O]
or the compound has the structure: 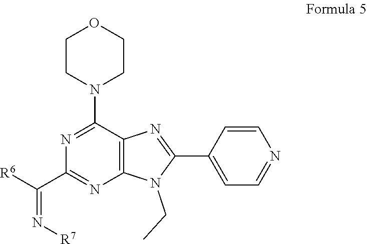
or a pharmaceutically acceptable salt thereof,
wherein R⁶ is hydrogen or methyl; and
R⁷ is optionally substituted phenoxy, optionally substituted benzyloxy, or optionally substituted amine;
or the compound has the structure: 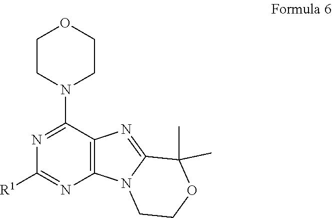
or a pharmaceutically acceptable salt thereof,
wherein R¹ is optionally substituted pyrazol-1-yl or —N(R^{1A})N═C(R^{1B})₂;
or the compound has the structure: 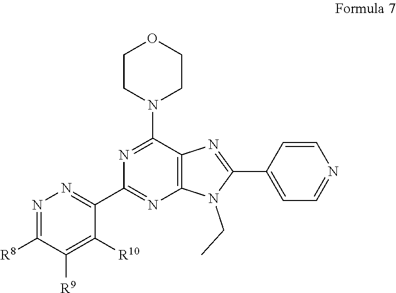
or a pharmaceutically acceptable salt thereof,
wherein R⁸ is hydrogen or methoxy; R⁹ is hydrogen or phenyl; and R¹⁰ is hydrogen or phenyl;
or the compound has the structure: 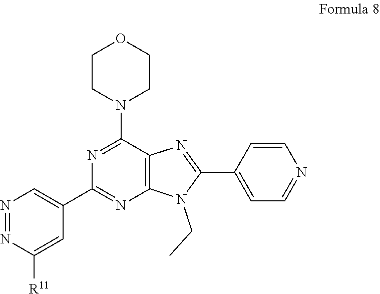
or a pharmaceutically acceptable salt thereof,
wherein R¹¹ is hydrogen or phenyl;
or the compound has the structure: 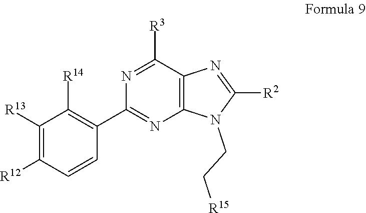
or a pharmaceutically acceptable salt thereof,
wherein R¹² is hydrogen, methoxy, or CH₂OH;
R¹³ is hydrogen, methoxy, C₃ cycloalkoxy, optionally substituted C₂-C₉ heteroaryl, optionally substituted C₂-C₉ heterocyclyl, or optionally substituted C₁-C₆ alkyl;
R¹⁴ is hydrogen or C₃ cycloalkoxy, or optionally substituted C₂-C₉ heteroaryl;
R¹⁵ is hydrogen or hydroxyl;
R² is hydrogen, pyridin-4-yl, 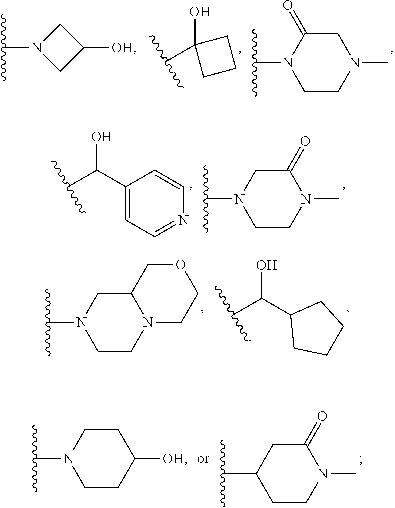
and
R³ is 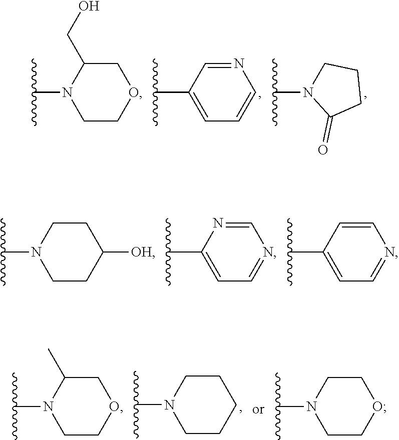
or the compound has the structure: 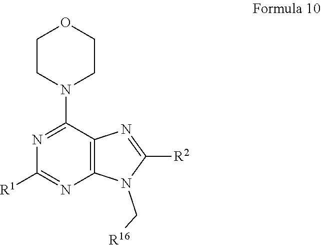
or a pharmaceutically acceptable salt thereof,
wherein R¹ is 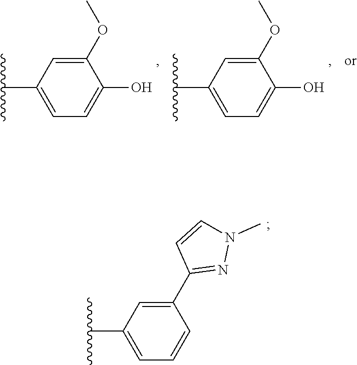
R¹⁶ is hydrogen or pyridine-3-yl; and
R² is pyridin-4-yl or hydrogen:
or the compound has the structure: 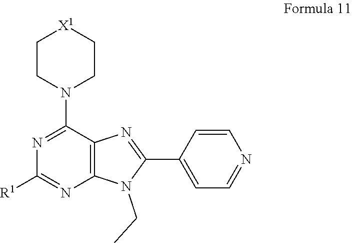
or a pharmaceutically acceptable salt thereof,
wherein X¹ is O or CH₂; and R¹ is —N(R^{1A})N═C(R^{1B})₂;
or the compound has the structure: 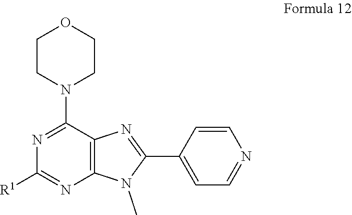
or a pharmaceutically acceptable salt thereof,
wherein R¹ is 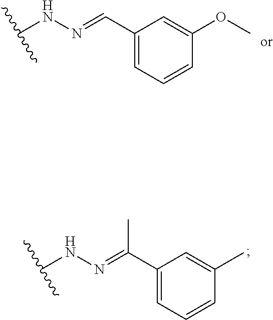 [SMILES: *N/N=C/c1cccc(OC)c1 ; *N/N=C(\C)c1cccc(C)c1]
or the compound has the structure: 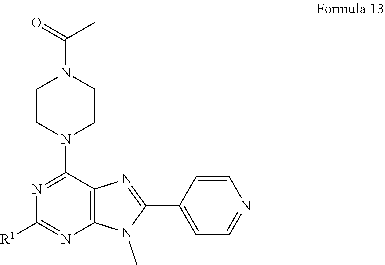
or a pharmaceutically acceptable salt thereof,
wherein R¹ is —N(R^{1A})N═C(R^{1B})₂;
or the compound has the structure: 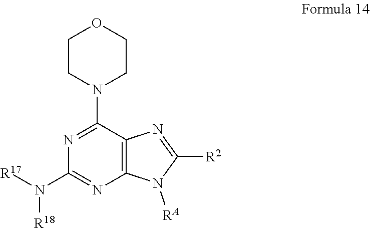
or a pharmaceutically acceptable salt thereof,
wherein R¹⁷ is optionally substituted C₆-C₁₀ aryl C₁-C₆ alkyl, optionally substituted C₆-C₁₀ heteroaryl C₁-C₆ alkyl, —NH₂, optionally substituted C₃-C₈ cycloalkyl, or optionally substituted C₂-C₉ heteroaryl;
R¹⁸ is hydrogen or optionally substituted C₁-C₆ alkyl;
R^{A} is methyl or ethyl; and
R² is pyridin-4-yl or hydrogen;
or the compound has the structure: 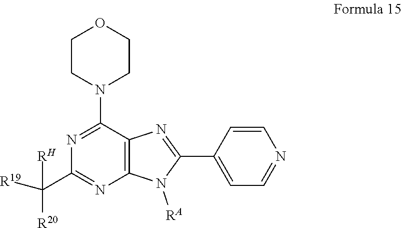
or a pharmaceutically acceptable salt thereof,
wherein R¹⁹ is optionally substituted amino, optionally substituted C₂-C₉ heterocycle, optionally substituted C₂-C₉ heteroaryl;
R^{H} and R²⁰, together with the atom to which they are attached, combine to form oxo;
R²⁰ is hydrogen, or R²⁰ and R^{H}, together with the atom to which they are attached, combine to form oxo; and
R^{A} is ethyl or cyclopropyl;
or the compound has the structure: 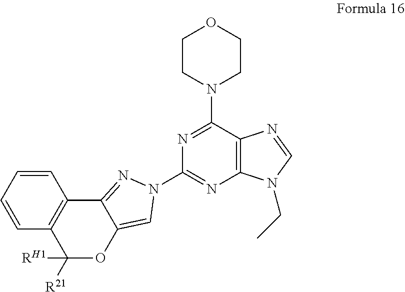
or a pharmaceutically acceptable salt thereof,
wherein R²¹ is hydrogen or R²¹ and R^{H1}, together with the atom to which they are attached, combine to form oxo; and
R^{H1} is hydrogen or R^{H1} and R²¹, together with the atom to which they are attached, combine to form oxo;
or the compound has the structure: 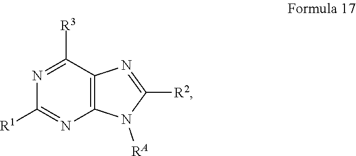
or a pharmaceutically acceptable salt thereof,
wherein R¹ is pyrazol-1-yl disubstituted with optionally substituted C₆-C₁₀ aryl; optionally substituted C₁-C₆ heteroalkyl; optionally substituted C₁-C₆ alkyl; optionally substituted C₂-C₉ heteroaryl, halo, hydroxy, optionally substituted C₃-C₈ cycloalkyl, or optionally substituted C₁-C₆ alkyl;
R³ is 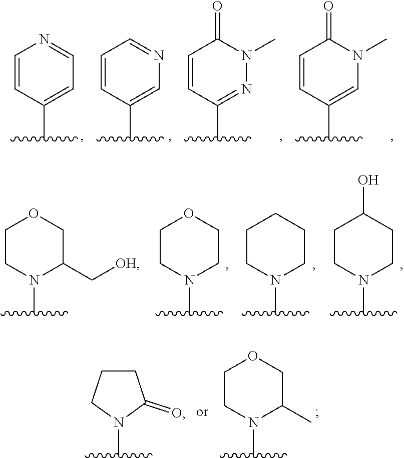
R^{A} is ethyl, 2-hydroxy-ethyl, methyl 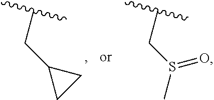
and R² is hydrogen, methyl, ethyl, halo, pyridin-3-yl, pyridin-4-yl, cyclopropyl, 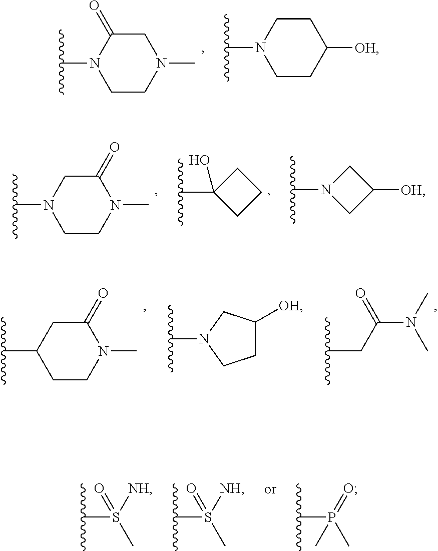
or R² and R^{A}, together with the atoms to which they are attached, combine to form an optionally substituted C₄ heterocyclyl:
or the compound has the structure: 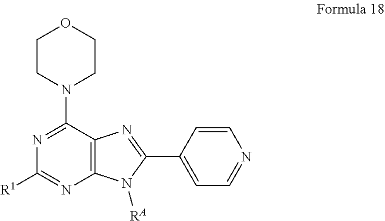
or a pharmaceutically acceptable salt thereof,
wherein R¹ is optionally substituted triazolyl; and R^{A} is methyl, ethyl, or cyclopropyl;
or the compound has the structure: 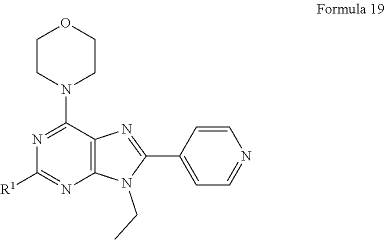
or a pharmaceutically acceptable salt thereof,
wherein R¹ is optionally substituted indazolyl or optionally substituted 4,5,6,7-tetrahydrotriazaindenyl:
or the compound has the structure: 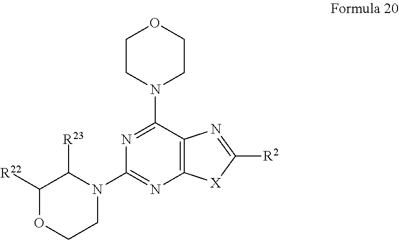
or a pharmaceutically acceptable salt thereof,
wherein X is S or NR^{A};
R²² is hydrogen or phenyl;
R²³ is hydrogen or methyl;
R² is pyrazol-3-yl, pyridine-4-yl, or 4-phenyl-pyrazol-1-yl; and
R^{A} is methyl;
or the compound has the structure: 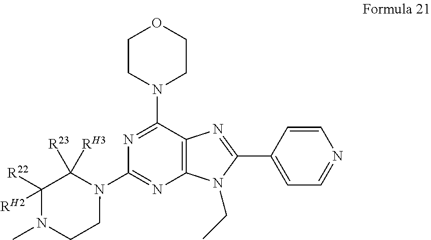
or a pharmaceutically acceptable salt thereof,
wherein R²² is phenyl, pyridine-2-yl, or R²² and R^{H2} together with the atom to which they are attached, combine to form oxo;
R^{H2} is hydrogen or R^{H2} and R²² together with the atom to which they are attached, combine to form oxo;
R²³ is hydrogen or R²³ and R^{H3}, together with the atom to which they are attached, combine to form oxo; and
R^{H3} is hydrogen or R^{H3} and R²³, together with the atom to which they are attached, combine to form oxo;
or the compound has the structure: 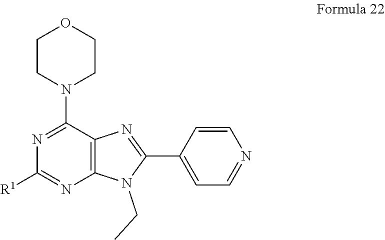
or a pharmaceutically acceptable salt thereof,
wherein R¹ is 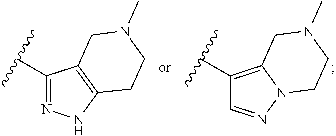 [SMILES: *c1n[nH]c2c1CN(C)CC2 ; *c1cnn2c1CN(C)CC2]
or the compound has the structure: 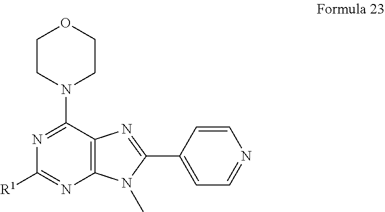
wherein R¹ is 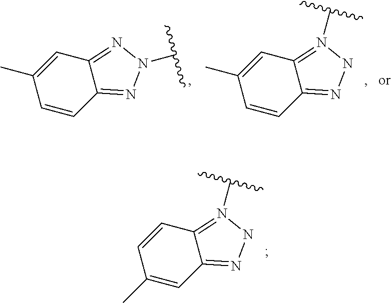
or the compound has the structure: 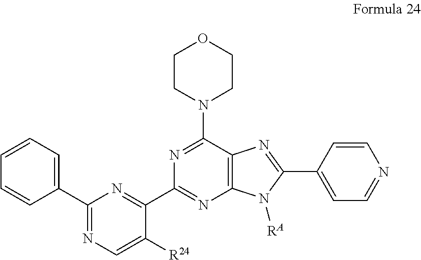
or a pharmaceutically acceptable salt thereof,
wherein R²⁴ is methoxy, methyl or hydroxyl; and R^{A} is methyl or ethyl;
or the compound has the structure: 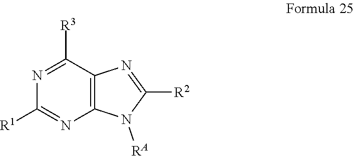
or a pharmaceutically acceptable salt thereof,
wherein R¹ is optionally substituted pyrazolyl, optionally substituted pyrimidin-3-yl, or optionally substituted pyridin-4-yl;
R^{A} is methyl or ethyl;
R² is optionally substituted C₂-C₉ heteroaryl, or optionally substituted C1-C9 heterocyclyl; and
R³ is 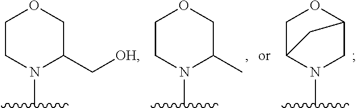
or the compound has the structure: 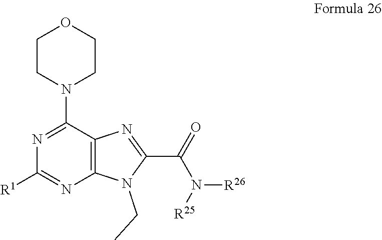
or a pharmaceutically acceptable salt thereof,
wherein R¹ is optionally substituted pyrazol-1-yl or phenyl substituted with optionally substituted C₂-C₉ heteroaryl; and
R²⁵ and R²⁶, together the atom to which they are attached, combine to form a C₃-C₅ heterocyclyl substituted with hydroxyl;
or the compound has the structure: 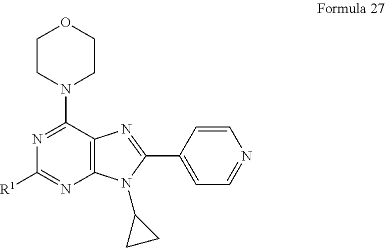
or a pharmaceutically acceptable salt thereof,
wherein R¹ is optionally substituted pyrazol-1-yl optionally substituted pyrazol-5-yl, or phenyl substituted with methoxy or C₃-C₈ cycloalkoxy;
or the compound has the structure: 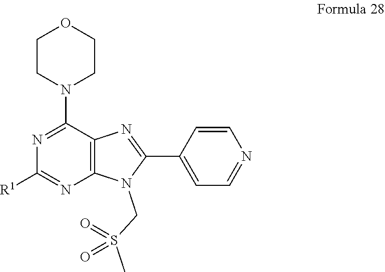
or a pharmaceutically acceptable salt thereof,
wherein R¹ is optionally substituted pyrazol-1-yl;
or the compound has the structure: 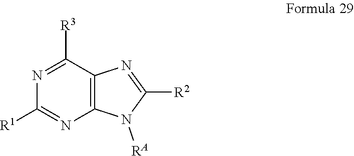
or a pharmaceutically acceptable salt thereof,
wherein R¹ is optionally substituted pyrazol-1-yl, optionally substituted pyrazol-3-yl, or optionally substituted pyrazol-5-yl;
R³ is morpholin-1-yl or piperidin-1-yl;
R^{A} is methyl or ethyl; and
R² is 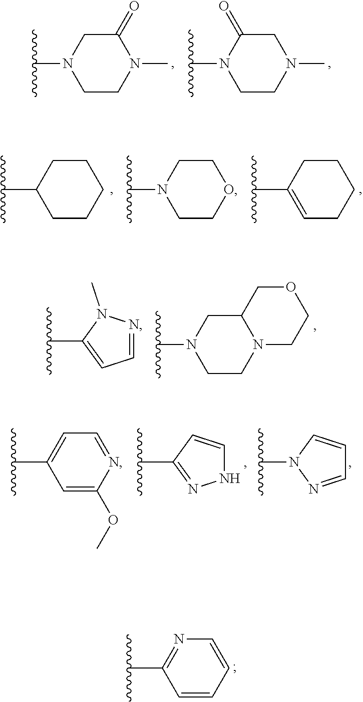
or the compound has the structure: 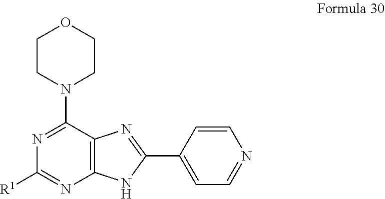
or a pharmaceutically acceptable salt thereof,
wherein R¹ is pyrazolyl monosubstituted with optionally substituted C₂-C₉ heterocyclyl or C₆-C₁₀ aryl;
or the compound has the structure: 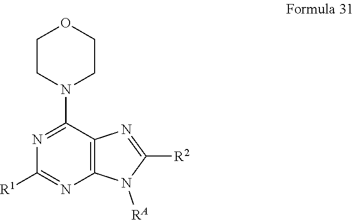
or a pharmaceutically acceptable salt thereof,
wherein R¹ is optionally substituted pyrazol-1-yl or pyrimidin-4-yl optionally substituted with optionally substituted C₁-C₆ alkyl;
R^{A} is methyl or difluoromethyl;
R² is pyridin-4-yl or 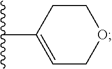 [SMILES: *C1=CCOCC1]
or the compound has the structure: 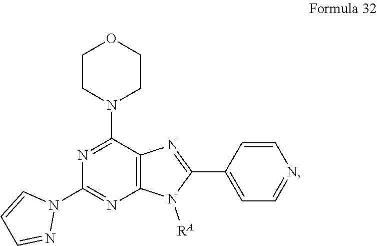
or a pharmaceutically acceptable salt thereof,
wherem R^{A} is 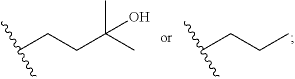
or the compound has the structure: 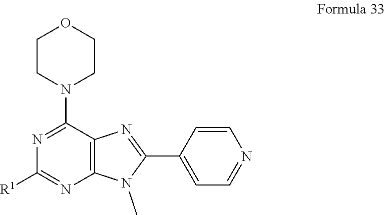
wherein R¹ is 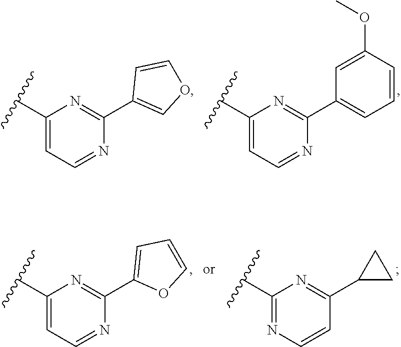
or the compound has the structure: 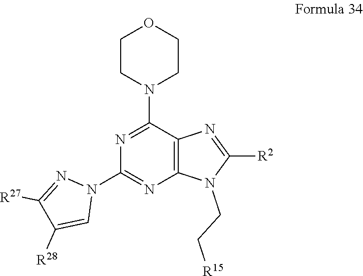
or a pharmaceutically acceptable salt thereof,
wherein R²⁷ is hydrogen, tetrahydropyran-3-yl, or tetrahydropyran-4-yl;
R²⁸ is hydrogen, methoxy, phenyl, methyl, difluoromethyl, optionally substituted cyclobutyl,
R¹⁵ is hydrogen or methoxy; and
R² is pyridin-4-yl or —O-pyridin-4-yl;
or the compound has the structure: 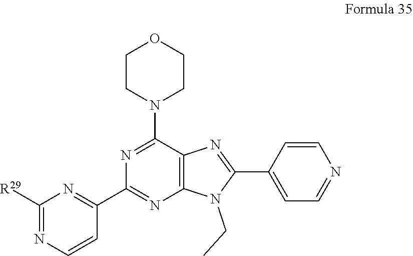
or a pharmaceutically acceptable salt thereof,
wherein R²⁹ is optionally substituted C₂-C₉ heterocyclyl or optionally substituted C₆-C₁₀ aryl;
or the compound has the structure: 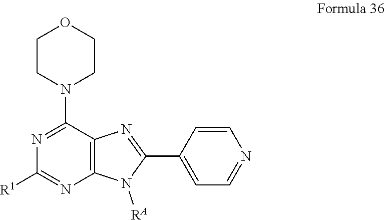
or a pharmaceutically acceptable salt thereof,
wherein R¹ is optionally substituted 4,5-dihydro-pyrazol-1-yl, optionally substituted imidazol-2-yl, optionally substituted piperidin-1-yl, or optionally substituted 1,2,4-triazol-3-yl, optionally substituted pyrazol-4-yl, optionally substituted 1,3,4-oxadiazol-2-yl, or optionally substituted pyridin-3-yl; and
R^{A} is methyl or ethyl;
or the compound has the structure: 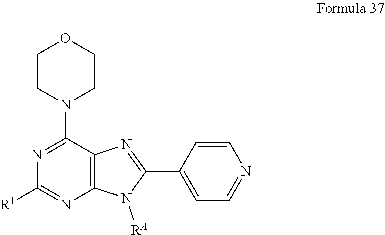
or a pharmaceutically acceptable salt thereof,
wherein R¹ is pyrazol-5-yl optionally substituted with C₂-C₉ heteroaryl, C₆-C₁₀ aryl, C₃-C₈ cycloalkyl or C₃-C₈ cycloalkyl C₁-C₆ alkyl; and
R^{A} is methyl or ethyl;
or the compound has the structure: 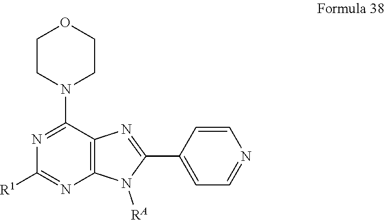
or a pharmaceutically acceptable salt thereof,
wherein R¹ is pyrazol-3-yl substituted with optionally substituted C₂-C₉ heteroaryl, optionally substituted C₃-C₈ cycloalkyl, optionally substituted C₂ alkyl, or optionally substituted C₆-C₁₀ aryl C₁-C₆ alkyl; and
R^{A} is methyl or ethyl;
or the compound has the structure: 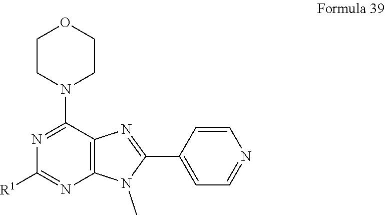
or a pharmaceutically acceptable salt thereof,
wherein R¹ is pyrazol-3-yl disubstituted with C₁-C₆ alkyl or C₆-C₁₀ aryl;
or the compound has the structure: 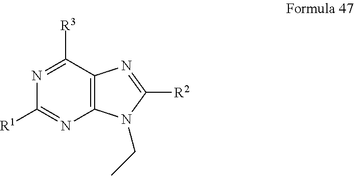
or a pharmaceutically acceptable salt thereof,
wherein R² is hydrogen, optionally substituted C₂-C₉ heteroaryl; optionally substituted C₂-C₉ heterocyclyl, or C₁-C₃ alkyl optionally substituted with hydroxyl, oxo, or dialkyl amino;
R¹ is optionally substituted pyrazol-1-yl, phenyl optionally substituted with optionally substituted C₂-C₉ heteroaryl or optionally substituted C₆-C₁₀ aryl, or —N(R^{1A})N═C(R^{1B})₂; and
R³ is 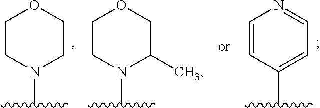
or the compound has the structure: 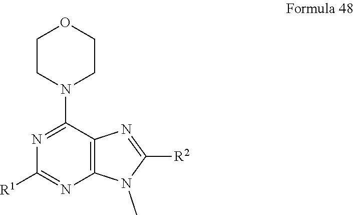
or a pharmaceutically acceptable salt thereof,
wherein R² is optionally substituted C₂-C₉ heteroaryl; and
R¹ is —N(R^{1A})N═C(R^{1B})₂.

32-258. (canceled)

259. A compound, or pharmaceutically acceptable salt thereof, having the structure: 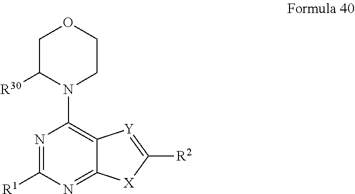
or a pharmaceutically acceptable salt thereof,
wherein Y is CH or N;
X is O, or S;
R¹ is optionally substituted morpholin-1-yl, optionally substituted pyrimidin-4-yl, —N(R^{1A})N═C(R^{1B})₂, optionally substituted pyrazol-3-yl, or optionally substituted indazol-4-yl;
R² is hydrogen or methyl; and
R³⁰ is optionally substituted pyridin-4-yl, optionally substituted pyrazol-3-yl, optionally substituted pyrazol-1-yl, or C₂-C₉ heterocycle C₁-C₆ alkyl substituted with —S(O)₂CH₃;
or having the structure: 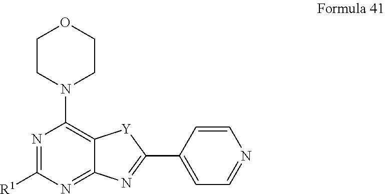
or a pharmaceutically acceptable salt thereof,
wherein Y is S or NR^{A};
R¹ is optionally substituted pyrimidin-4-yl; and
R⁴ is optionally substituted C₁-C₆ alkyl;
or having the structure: 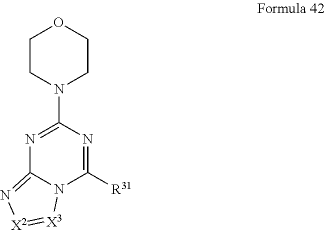
or a pharmaceutically acceptable salt thereof,
wherein X² and X³ are each, independently, N or CR³²;
R³¹ is optionally substituted C₂-C₉ heteroaryl; and
R³² is optionally substituted C₂-C₉ heteroaryl;
or having the structure: 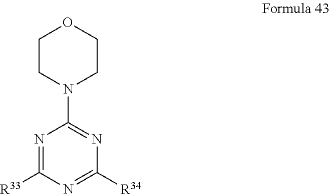
or a pharmaceutically acceptable salt thereof,
wherein R³³ is optionally substituted amino; and
R³⁴ is optionally substituted C₂-C₉ heteroaryl;
or having the structure: 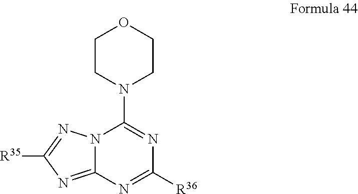
or a pharmaceutically acceptable salt thereof,
wherein R³⁵ and R³⁶ are each, independently, optionally substituted C₂-C₉ heteroaryl;
or having the structure: 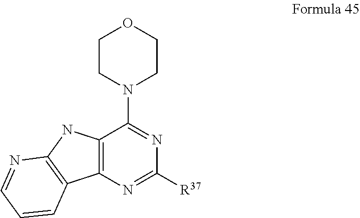
or a pharmaceutically acceptable salt thereof,
wherein R³⁷ is optionally substituted C₂-C₉ heteroaryl;
or having the structure: 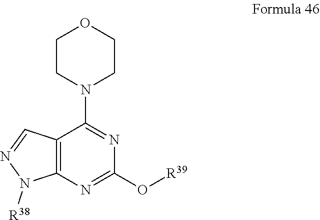
or a pharmaceutically acceptable salt thereof,
wherein R³⁸ is optionally substituted C₆-C₁₀ aryl; and
R³⁹ is optionally substituted C₂-C₉ heteroaryl C₁-C₆ alkyl.

260-332. (canceled)

333. A compound having the structure of any one of compounds 1-476 in Table 1, or a pharmaceutically acceptable salt thereof.

334. (canceled)

335. A pharmaceutical composition comprising the compound of claim 1, or a pharmaceutically acceptable salt thereof, and a pharmaceutically acceptable excipient.

336. A method of treating a neurological disorder in a subject in need thereof, the method comprising administering to the subject an effective amount of the compound of claim 1, or a pharmaceutically acceptable salt thereof.

337. The method of claim 336, wherein the neurological disorder is FTLD-TDP, chronic traumatic encephalopathy, ALS, Alzheimer's disease, LATE, or frontotemporal lobar degeneration.

338. (canceled)

339. A method of inhibiting toxicity in a cell related to a protein, the method comprising contacting the cell with the compound of claim 1 or a pharmaceutically acceptable salt thereof.

340. The method of claim 339, wherein the toxicity is TDP-43-related toxicity, or C9orf72-related toxicity.

341. (canceled)

342. A method of inhibiting PIKfyve in a cell expressing PIKfyve protein, the method comprising contacting the cell with the compound of claim 1 or a pharmaceutically acceptable salt thereof.

343-347. (canceled)
